(19) United States
(12) Patent Application Publication
(10) Pub. No.: US 2026/0317320 A1
(43) Pub. Date: Oct. 15, 2026

(54) C5AR1 ANTAGONISTS AND USES THEREOF

(71) Applicant: Vanqua Bio, Inc., Chicago, IL (US)

(72) Inventors: Kevin Hunt, Chicago, IL (US); Jianbin Zheng, Chicago, IL (US); Sida Shen, Chicago, IL (US)

(73) Assignee: Vanqua Bio, Inc., Chicago, IL (US)

(21) Appl. No.: 19/110,738

(22) PCT Filed: Sep. 13, 2023
(86) PCT No.: PCT/US2023/032581

§ 371 Date: Mar. 11, 2025

Related U.S. Application Data
(60) Provisional application No. 63/406,501, filed on Sep. 14, 2022.

(51) Int. Cl. C07D 471/04; A61K 31/4375; A61K 31/444; A61K 31/497; A61K 31/498; A61K 31/4985; A61K 31/501; A61K 31/506; A61K 31/517; A61K 31/519; C07D 401/14; C07D 487/04; C07D 519/00

(52) U.S. Cl. CPC C07D 471/04; A61K 31/4375; A61K 31/444; A61K 31/497; A61K 31/498; A61K 31/4985; A61K 31/501; A61K 31/506; A61K 31/517; A61K 31/519; C07D 401/14; C07D 487/04; C07D 519/00

(57) ABSTRACT

Provided herein are compounds that modulate (e.g., antagonize) complement component 5a receptor 1 (C5aR1), a G protein-coupled receptor for C5a that is associated with autoimmune, inflammatory, and neurodegenerative disorders. Also provided are pharmaceutical compositions and kits comprising the compounds, and methods of treating C5aR1-related diseases and disorders with the compounds in a subject, by administering the compounds and/or compositions described herein.

### RELATED APPLICATION

This application claims priority under 35 U.S.C. § 119(e) to U.S. Provisional Application 63/406,501 filed Sep. 14, 2022, the entirety of which is incorporated herein by reference.

### BACKGROUND

The C5a receptor, also known as complement component 5a receptor 1 (C5aR1) or CD88 (Cluster of Differentiation 88) is a G protein-coupled receptor for C5a. It functions as a complement receptor.

C5a is a potent immunomodulator, but also has emerging roles outside of immunity during embryonic development. Its production is tightly controlled in order to produce a rapid response to tissue injury/pathogens yet protect against unwanted activity on host tissue. The disruption of this control can lead to the overproduction of C5a, which can lead to an uncontrolled inflammatory response. C5a has been associated with a number of autoimmune, inflammatory, and neurodegenerative disorders. Thus, targeting C5aR1 with effective small molecule modulators would be useful in the treatment of several types of diseases (e.g., neurodegenerative disorders).

### SUMMARY

The present disclosure provides compounds that are modulators of C5AR1 (e.g., antagonists). These compounds provide new compositions and methods for the treatment of diseases associated with C5AR1 activity.

In one aspect, provided are compounds of Formula (I):

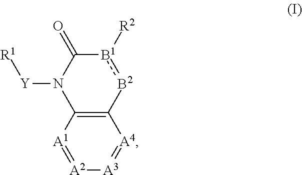

and pharmaceutically acceptable salts, co-crystals, tautomers, stereoisomers, solvates, hydrates, polymorphs, isotopically enriched derivatives, or prodrugs thereof, wherein:
R¹ is an optionally substituted aryl, optionally substituted heteroaryl, optionally substituted carbocyclyl, or optionally substituted heterocyclyl;
R² is an optionally substituted carbocyclyl or optionally substituted heterocyclyl;
A¹, A², A³, and A⁴ are each independently ═C(R³)— or ═N—;
B¹ is —C(R⁴)ₜ— or —N—, wherein t is 0 or 1 as valency permits;
B² is ═C(R⁴)—, —N—, or —C(O)—;
Y is —C(R⁵)₂— or a bond;
each R³ is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A};
each R⁴ is independently hydrogen, halogen, optionally substituted alkyl, optionally substituted alkenyl, optionally substituted alkynyl, —OR^{A}, —N(R^{A})₂, —SR^{A}, or —CN;
each R⁵ is independently hydrogen, halogen, or optionally substituted alkyl; and
each R^{A} is independently hydrogen, optionally substituted acyl, optionally substituted alkyl, optionally substituted alkenyl, optionally substituted alkynyl, optionally substituted heteroaliphatic, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom.

In certain embodiments, the compounds of Formula (I) are compounds of Formula (I-a), (I-a-1), (I-a-2), (I-a-3), (I-a-4), (I-a-5), (I-a-6), (I-a-7), (I-a-8), (I-a-9), (I-a-10), (I-a-11), (I-b), (I-c), (I-d), (I-e), (I-e-1), (I-e-2), (I-e-3), (I-e-4), (I-e-5), (I-e-6), (I-e-7), (I-e-8), (I-f), (I-f-1), (I-f-2), (I-g), (I-g-1), (I-g-2), (I-h), (I-h-1), (I-h-2), (I-h-7), (I-h-8), or (I-h-9):

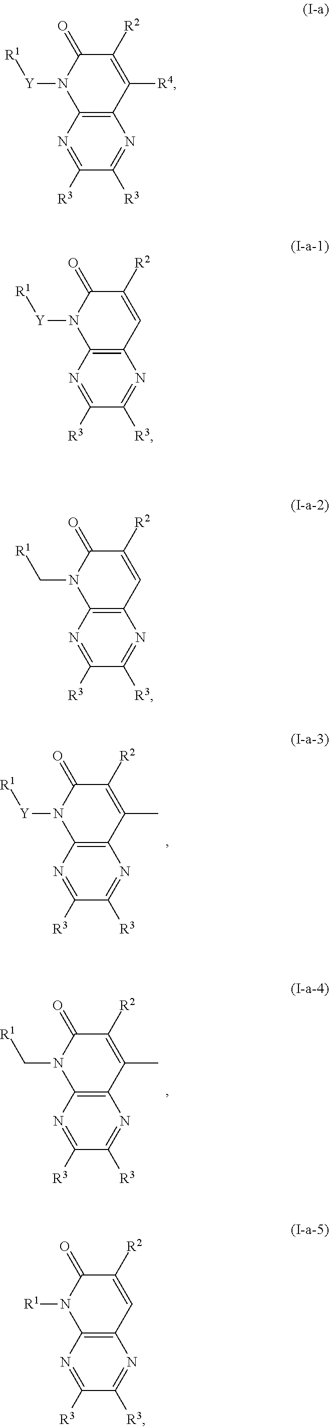

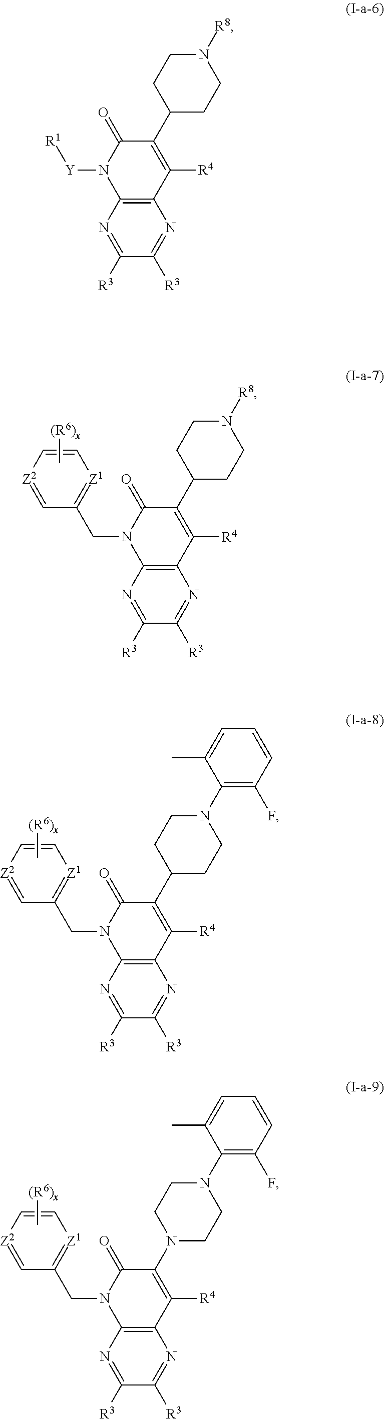

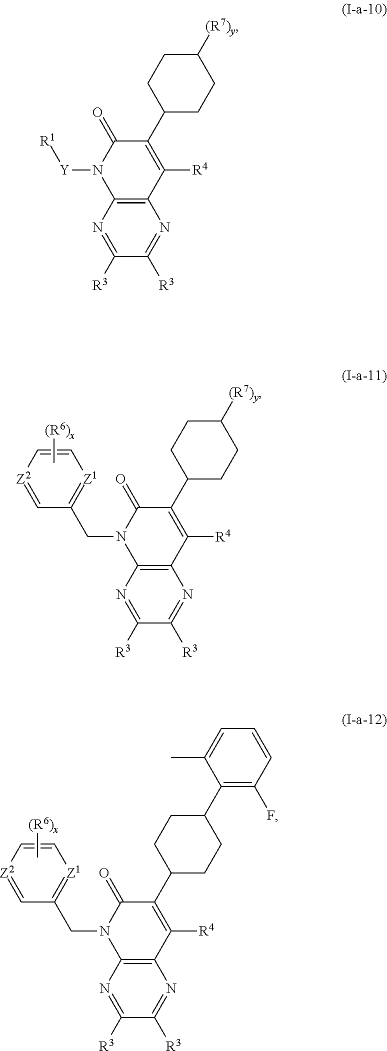

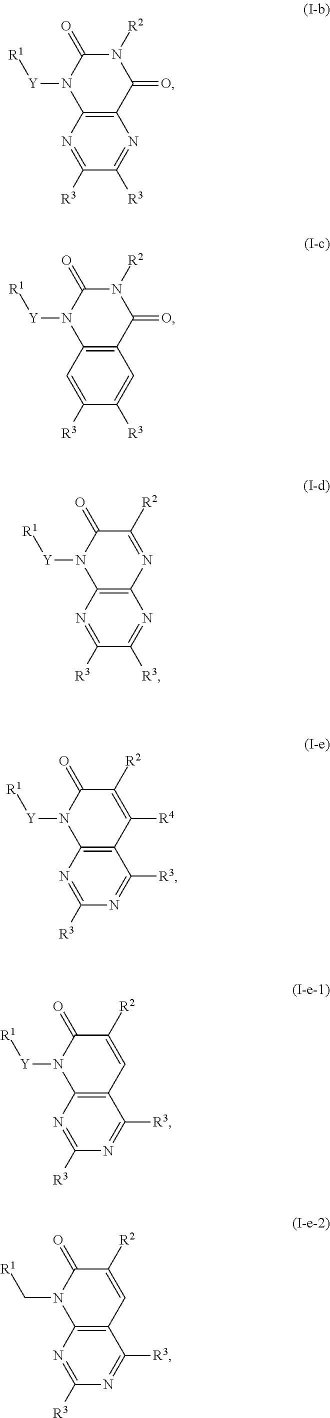

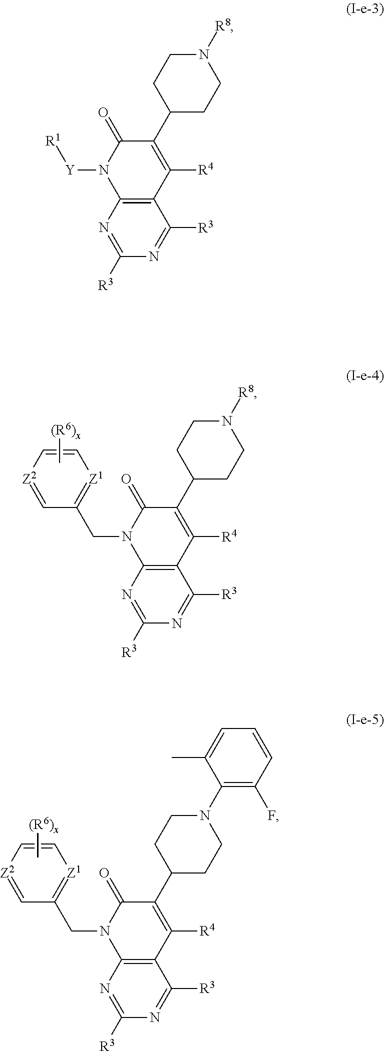

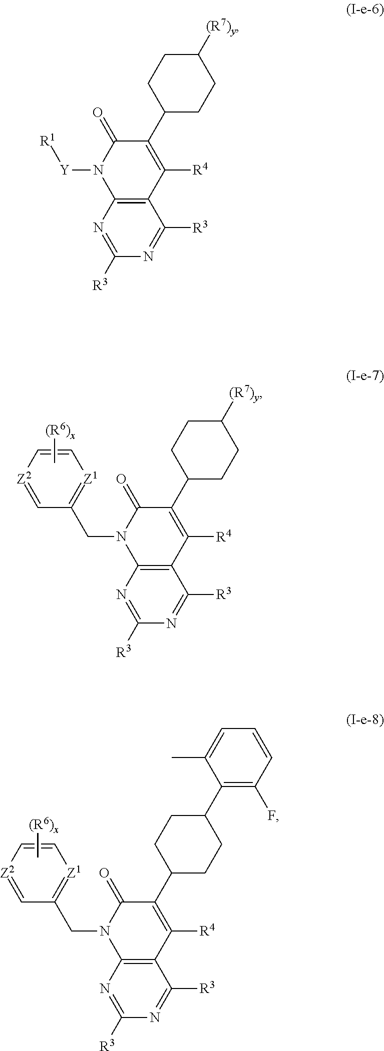

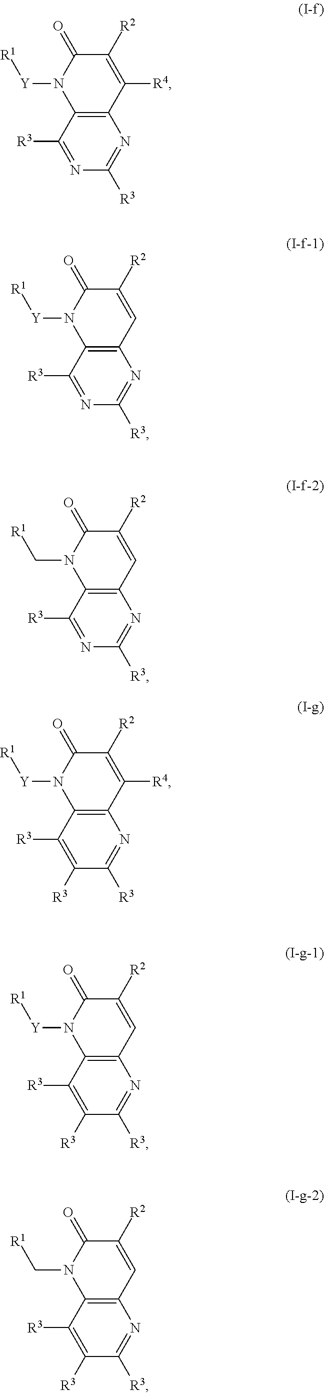

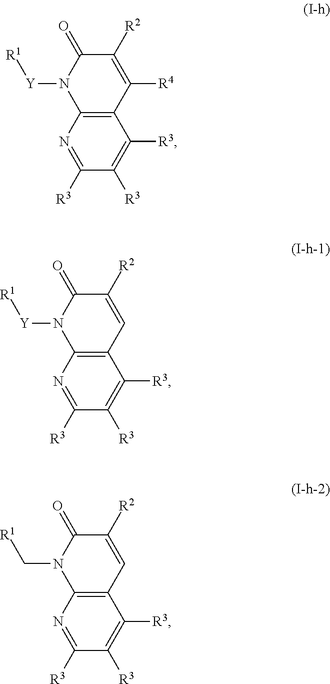

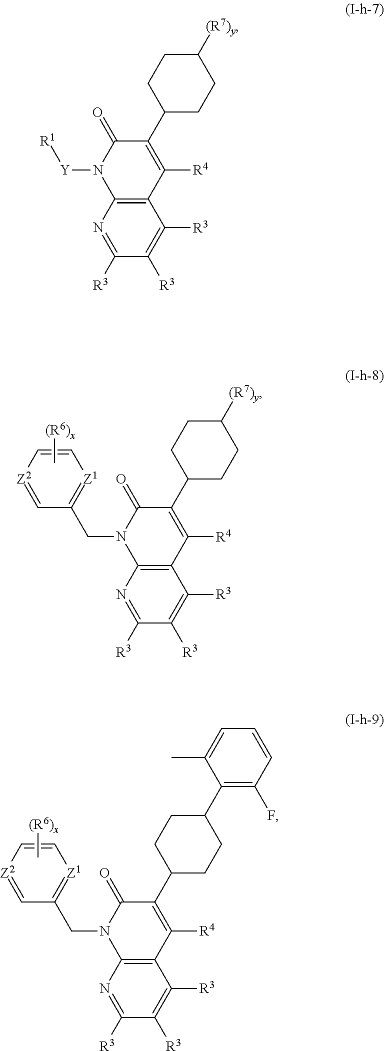

or pharmaceutically acceptable salts thereof.

In another aspect, provided are pharmaceutical compositions comprising a compound of Formula (I), or a pharmaceutically acceptable salt thereof, and optionally a pharmaceutically acceptable excipient.

In another aspect, provided are methods of treating a disease or disorder in a subject in need thereof, the method comprising administering a compound of Formula (I), or a pharmaceutically acceptable salt thereof, or a pharmaceutical composition comprising a compound of Formula (I) to the subject.

In certain embodiments, the disease or disorder is associated with C5aR1 (e.g., C5aR1 activity). In certain embodiments, the disease or disorder is cancer, an infectious disease, an autoimmune disease or disorder, an inflammatory disease or disorder, a cardiovascular disease or disorder, a cerebrovascular disease or disorder, a vasculitis disease or disorder, or a neurodegenerative disease or disorder. In certain embodiments, the disease or disorder is a neurological disease or disorder.

In another aspect, provided are methods of antagonizing C5aR1, the method comprising contacting C5aR1 with an effective amount of a compound of Formula (I), or a pharmaceutically acceptable salt thereof, or a pharmaceutical composition comprising a compound of Formula (I) to the subject.

In another aspect, provided are kits comprising a compound of Formula (I), or a pharmaceutically acceptable salt thereof, or a pharmaceutical composition comprising a compound of Formula (I), or a pharmaceutically acceptable salt thereof. In certain embodiments, the kits further comprise instructions for administration (e.g., human administration).

The details of certain embodiments of the disclosure are set forth in the Detailed Description of Certain Embodiments, as described below. Other features, objects, and advantages of the disclosure will be apparent from the Definitions, Examples, and Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers. While compounds may be depicted as racemic or as one or more diastereoisomers, enantiomers, or other isomers, all such racemic, diastereoisomer, enantiomer, or other isomer forms of that depicted are included in the present disclosure.

In a formula, [symbol] is a single bond where the stereochemistry of the moieties immediately attached thereto is not specified, [symbol] is absent or a single bond, and [symbol] or - - - is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C_{4_5}, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (Cs), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋ₛ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “alkoxy” refers to an alkyl group, as defined herein, appended to the parent molecular moiety through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋ₛ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”).

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

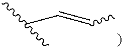

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂-8 alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and lor 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Arylalkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroarylalkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

When substituted, exemplary carbon atom substituents include, but are not limited to, halogen, —CN, —NO₂, —N₃, —SO₂H, —SO₃H, —OH, —OR—, —ON(R^{bb})₂, —N(R^{bb})₂, —N(R^{bb})₃⁺X⁻, —N(OR^{cc})R^{bb}, —SH, —SR^{aa}—, —SSR^{cc}, —SF₅, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{aa})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), —R^{aa}, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula: —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, or —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined herein.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{a}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

These and other exemplary substituents are described in more detail in the Detailed Description, Examples, and Claims. The invention is not intended to be limited in any manner by the above exemplary listing of substituents.

### Other Definitions

The term “antagonist” refers to an agent that (i) decreases or suppresses one or more effects of another agent; and/or (ii) decreases or suppresses one or more biological events. In some embodiments, an antagonist may reduce level and/or activity or one or more agents that it targets. An antagonist may be direct (in which case it exerts its influence directly upon its target) or indirect (in which case it exerts its influence by other than binding to its target; e.g., by interacting with a regulator of the target, for example so that level or activity of the target is altered). In some embodiments, an antagonist may be a receptor antagonist, e.g., a receptor ligand or drug that blocks or dampens a biological response by binding to and blocking a receptor rather than activating it like an agonist. In certain embodiments, compounds of Formula (I) are antagonists of C5aR1.

The term “agonist” refers to an agent that (i) increases or induces one or more effects of another agent; and/or (ii) increases or induces one or more biological events. In some embodiments, an agonist may increase level and/or activity or one or more agents that it targets. In various embodiments, agonists may be or include agents of various chemical class including, for example, small molecules, polypeptides, nucleic acids, carbohydrates, lipids, metals, and/or other entity that shows the relevant agonistic activity. An agonist may be direct (in which case it exerts its influence directly upon its target) or indirect (in which case it exerts its influence by other than binding to its target; e.g., by interacting with a regulator of the target, for example so that level or activity of the target is altered). A partial agonist can act as a competitive antagonist in the presence of a full agonist, as it competes with the full agonist to interact with its target and/or a regulator thereof, thereby producing (i) a decrease in one or more effects of another agent, and/or (ii) a decrease in one or more biological events, as compared to that observed with the full agonist alone.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and/or animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water molecules. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). Many compounds can adopt a variety of different crystal forms (i.e., different polymorphs). Typically, such different crystalline forms have different X-ray diffraction patterns, infrared spectra, and/or can vary in some or all properties such as melting points, density, hardness, crystal shape, optical and electrical properties, stability, solubility, and bioavailability. Recrystallization solvent, rate of crystallization, storage temperature, and other factors may cause one crystal form to dominate a given preparation. Various polymorphs of a compound can be prepared by crystallization under different conditions.

The term “co-crystal” refers to a crystalline structure composed of at least two components. In certain embodiments, a co-crystal contains a compound of the present disclosure and one or more other component(s), including, but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including, but not limited to, an isomer, tautomer, salt, solvate, hydrate, synthetic precursor, synthetic derivative, fragment, or impurity of said compound.

The term “prodrugs” refers to compounds that have cleavable groups that are removed, by solvolysis or under physiological conditions, to provide the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

The term “modulate” means decreasing or inhibiting activity and/or increasing or augmenting activity. For example, modulating C5aR1 activity means decreasing or inhibiting C5aR1 activity and/or increasing or augmenting C5aR1 activity. The compounds disclosed herein may be administered to modulate C5aR1 for example, as an antagonist.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, or inhibiting the progress of a disease described herein. In some embodiments, treatment may be administered after one or more signs or symptoms of the disease have developed or have been observed. Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for C5aR1 deactivation (e.g., at least 5%, at least 10%, at least 20%, at least 30%, at least 40%, at least 50%, at least 60%, at least 70%, at least 80%, at least 90%, at least 95%, at least 100%, at least 150%, at least 200%, at least 250%, at least 300%, or at least 500% decrease in the activity of C5aR1). In certain embodiments, a therapeutically effective amount is an amount sufficient for treating a disease or disorder (e.g., neurological disorder). In certain embodiments, a therapeutically effective amount is an amount sufficient for C5aR1 deactivation and treating a disease or disorder (e.g., neurological disorder).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for C5aR1 deactivation. In certain embodiments, a prophylactically effective amount is an amount sufficient for treating a disease or disorder (e.g., neurological disorder). In certain embodiments, a prophylactically effective amount is an amount sufficient for C5aR1 deactivation and treating a disease or disorder (e.g., neurological disorder).

As used herein, the term “activate” or “activation” in the context of proteins, for example, in the context of C5aR1, refers to an increase in the activity of the protein. In some embodiments, the term refers to an increase of the level of activity to a level that is statistically significantly higher than an initial level, which may, for example, be a baseline level of activity (e.g., of wild-type C5aR1). In some embodiments, the term refers to an increase in the level of activity to a level that is greater than 1%, greater than 5%, greater than 10%, greater than 25%, greater than 50%, greater than 75%, greater than 100%, greater than 150%, greater than 200%, greater than 300%, greater than 400%, greater than 500%, or greater than 1000% of an initial level, which may, for example, be a baseline level of activity.

The term “immunotherapy” refers to a therapeutic agent that promotes the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Immunotherapies are typically, but not always, biotherapeutic agents. Numerous immunotherapies are used to treat cancer. These include, but are not limited to, monoclonal antibodies, adoptive cell transfer, cytokines, chemokines, vaccines, and small molecule inhibitors.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics, or combinations thereof.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

### DETAILED DESCRIPTION OF CERTAIN EMBODIMENTS

Provided herein are compounds (e.g., a compound of any of the formulae herein) that are modulators of C5aR1 (e.g., C5aR1 antagonists). In one aspect, the provided C5aR1 modulators are compounds of Formula (I), and pharmaceutically acceptable salts, solvates, hydrates, polymorphs, co-crystals, tautomers, stereoisomers, isotopically labeled derivatives, prodrugs, and pharmaceutical compositions thereof. Accordingly, the compounds are useful for the treatment and/or prevention of diseases and disorders associated with C5aR1 (e.g., neurological diseases and disorders) in a subject in need thereof.

The compounds described herein interact with C5aR1. As described herein, the therapeutic effect may be a result of modulation (e.g., antagonism), binding, and/or modification of C5aR1 by the compounds described herein. The compounds may be provided for use in any composition, kit, or method described herein as a pharmaceutically acceptable salt, co-crystal, tautomer, stereoisomer, solvate, hydrate, polymorph, isotopically enriched derivative, or prodrug thereof.

### Compounds of Formula (I)

In one aspect, disclosed is a compound of Formula (I):

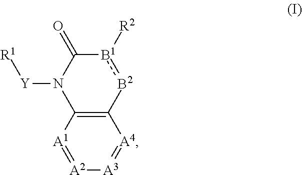

or a pharmaceutically acceptable salt, co-crystal, tautomer, stereoisomer, solvate, hydrate, polymorph, isotopically enriched derivative, or prodrug thereof, wherein:
R¹ is an optionally substituted aryl, optionally substituted heteroaryl, optionally substituted carbocyclyl, or optionally substituted heterocyclyl;
R² is an optionally substituted carbocyclyl or optionally substituted heterocyclyl;
A¹, A², A³, and A⁴ are each independently ═C(R³)— or ═N—;
B¹ is —C(R⁴)ₜ— or —N—, wherein t is 0 or 1 as valency permits;
B² is ═C(R⁴)—, —N—, or —C(O)—;
Y is —C(R⁵)₂— or a bond;
each R³ is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A};
each R⁴ is independently hydrogen, halogen, optionally substituted alkyl, optionally substituted alkenyl, optionally substituted alkynyl, —OR^{A}, —N(R^{A})₂, —SR^{A}, or —CN;
each R⁵ is independently hydrogen, halogen, or optionally substituted alkyl; and
each R^{A} is independently hydrogen, optionally substituted acyl, optionally substituted alkyl, optionally substituted alkenyl, optionally substituted alkynyl, optionally substituted heteroaliphatic, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom.

### Y

As described herein, Y is —C(R⁵)₂— or a bond; and each R⁵ is independently hydrogen, halogen, or optionally substituted alkyl. In certain embodiments, Y is —C(R⁵)₂—. In certain embodiments, Y is —C(R⁵)₂—; and each R⁵ is independently hydrogen or optionally substituted alkyl. In certain embodiments, Y is —CH₂—. In certain embodiments, Y is a bond.

### R¹

As described herein, R¹ is optionally substituted aryl, optionally substituted heteroaryl, optionally substituted carbocyclyl, or optionally substituted heterocyclyl. In certain embodiments, R¹ is optionally substituted aryl, optionally substituted heteroaryl, or optionally substituted carbocyclyl. In certain embodiments, R¹ is optionally substituted aryl or optionally substituted heteroaryl.

In certain embodiments, R¹ is optionally substituted aryl. In certain embodiments, R¹ is optionally substituted phenyl.

In certain embodiments, R¹ is optionally substituted heteroaryl. In certain embodiments, R¹ is optionally substituted pyridinyl, optionally substituted pyrazinyl, optionally substituted pyrimidinyl, or optionally substituted pyridazinyl. In certain embodiments, R¹ is optionally substituted pyridinyl or optionally substituted pyrazinyl. In certain embodiments, R¹ is optionally substituted phenyl, optionally substituted pyridinyl, or optionally substituted pyrazinyl. In certain embodiments, R¹ is optionally substituted pyridinyl. In certain embodiments, R¹ is optionally substituted pyrazinyl.

In certain embodiments, R¹ is

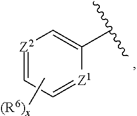

wherein Z¹ and Z² are each independently CH, CR⁶, or N; each R⁶ is independently halogen, optionally substituted alkyl, optionally substituted cycloalkyl, —SF₅, or —OR^{A}; and x is 0, 1, 2, or 3.

In certain embodiments, R¹ is

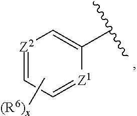

wherein Z¹ and Z² are each independently CH, CR⁶, or N; each R⁶ is independently halogen, optionally substituted alkyl, —SF₅, or —OR^{A}; and x is 0, 1, 2, or 3.

In certain embodiments, R¹ is

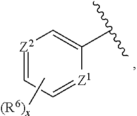

wherein Z¹ and Z² are each independently CH, CR⁶, or N; each R⁶ is independently halogen, optionally substituted alkyl, optionally substituted cycloalkyl, —SF₅, or —OR^{A}; and x is 0 or 1.

In certain embodiments, R¹ is

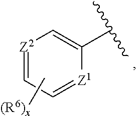

wherein Z¹ and Z² are each independently CH, CR⁶, or N; each R⁶ is independently halogen, optionally substituted alkyl, —SF₅, or —OR^{A}; and x is 0 or 1.

In certain embodiments, R¹ is

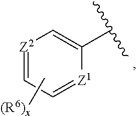

wherein Z¹ and Z² are each independently CH, CR⁶, or N; each R⁶ is independently halogen, alkyl, haloalkyl, optionally substituted cycloalkyl, —SF₅, or —OR^{A}; R^{A} is alkyl or haloalkyl; and x is 0 or 1.

In certain embodiments, R¹ is

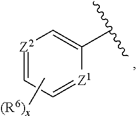

wherein Z¹ and Z² are each independently CH, CR⁶, or N; each R⁶ is independently halogen, alkyl, haloalkyl, —SF₅, or —OR^{A}; R^{A} is alkyl or haloalkyl; and x is 0 or 1.

In certain embodiments, R¹

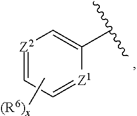

wherein Z¹ and Z² are each independently CH, CR⁶, or N; each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CHF₂, —OCF₃, or —OCHF₂; and x is 0 or 1.

In certain embodiments, R¹ is

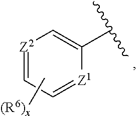

wherein Z¹ and Z² are each independently CH, CR⁶, or N; each R⁶ is independently —F, —SF₅, —CH₃, —CF₃, —CHF₂, —OCH₂CF₃, —OCH₃, —OCF₃, or —OCHF₂; and x is 0 or 1.

In certain embodiments, R¹ is

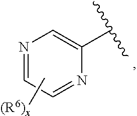

wherein each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CHF₂, —OCF₃, or —OCHF₂; and x is 0 or 1.

In certain embodiments, R¹ is

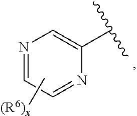

wherein each R⁶ is independently —F, —SF₅, —CH₃, —CF₃, —CHF₂, —OCH₂CF₃, —OCH₃, —OCF₃, or —OCHF₂; and x is 0 or 1.

In certain embodiments, R¹ is

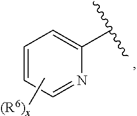

wherein each R⁶ is independently haloalkyl or optionally substituted cycloalkyl; and x is 0 or 1.

In certain embodiments, R¹ is

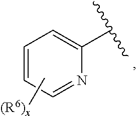

wherein each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCF₃, —OCH₂CHF₂, or —OCHF₂; and x is 0 or 1.

In certain embodiments, R¹ is

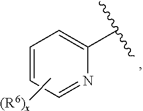

wherein each R⁶ is independently —F, —SF₅, —CH₃, —CF₃, —CHF₂, —OCH₂CF₃, —OCH₃, —OCF₃, or —OCHF₂; and x is 0 or 1.

In certain embodiments, R¹ is

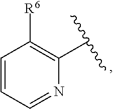

wherein each R⁶ is independently halogen, alkyl, haloalkyl, optionally substituted cycloalkyl, —SF₅, or —OR^{A}; and R^{A} is alkyl or haloalkyl.

In certain embodiments, R¹ is

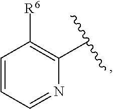

wherein each R⁶ is independently haloalkyl or optionally substituted cycloalkyl.

In certain embodiments, R¹

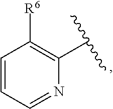

wherein each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CHF₂, —OCF₃, or —OCHF₂.

In certain embodiments, R¹ is

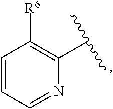

wherein each R⁶ is independently cyclopropyl, —CF₃, —CF₂CH₃, or —CH₂CF₃.

In certain embodiments, R¹ is

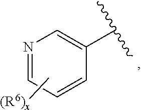

wherein each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CHF₂, —OCF₃, or —OCHF₂; and x is 0 or 1.

In certain embodiments, R¹

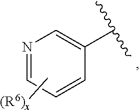

wherein each R⁶ is independently —F, —SF₅, —CH₃, —CF₃, —CHF₂, —OCH₂CF₃, —OCH₃, —OCF₃, or —OCHF₂; and x is 0 or 1.

In certain embodiments, R¹

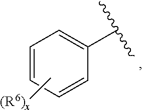

wherein each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CHF₂, —OCF₃, or —OCHF₂; and x is 0 or 1.

In certain embodiments, R¹ is

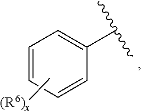

wherein each R⁶ is independently —F, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CHF₂, —OCF₃, or —OCHF₂; and x is 0 or 1.

In certain embodiments, R¹ is

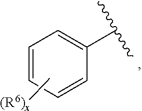

wherein each R⁶ is independently —F, —SF₅, —CH₃, —CF₃, —CHF₂, —OCH₂CF₃, —OCH₃, —OCF₃, or —OCHF₂; and x is 0 or 1.

In certain embodiments, R¹ is

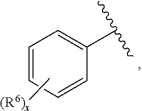

wherein each R⁶ is independently cyclopropyl, —CF₃, —CF₂CH₃, or —CH₂CF₃; and x is 0 or 1.

In certain embodiments, R¹ is

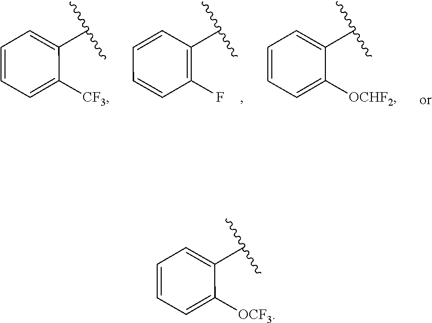 [SMILES: *c1ccccc1C(F)(F)F ; *c1ccccc1F ; *c1ccccc1OC(F)F ; *c1ccccc1OC(F)(F)F]

In certain embodiments, R¹ is

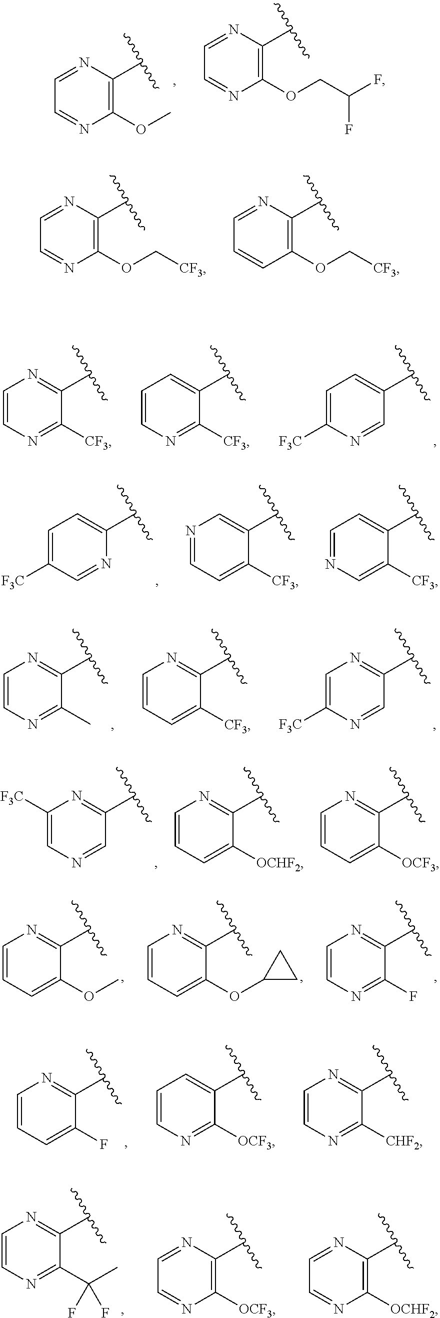

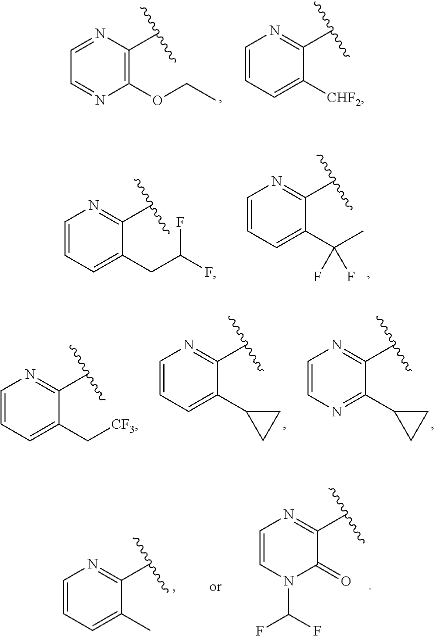

In certain embodiments, R¹ is

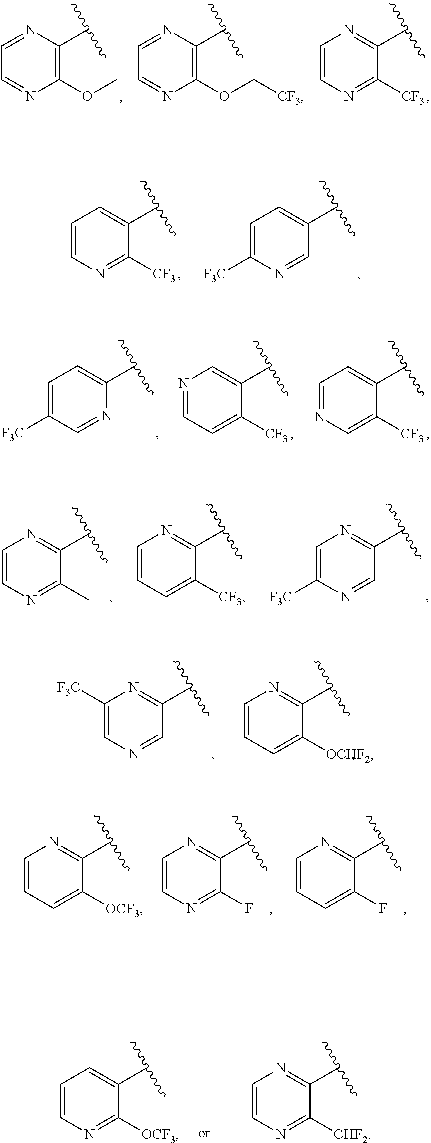

In certain embodiments, R¹ is

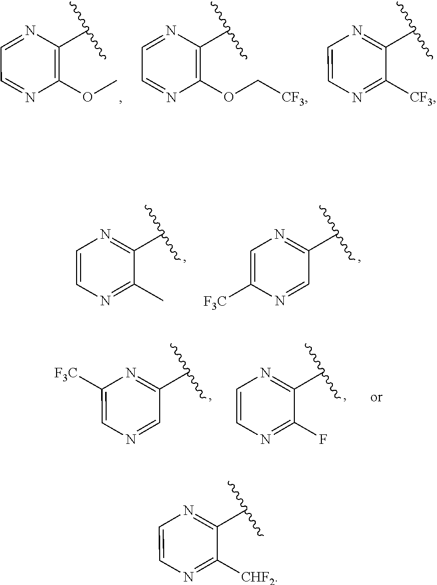

In certain embodiments, R¹ is

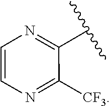 [SMILES: *c1nccnc1C(F)(F)F]

In certain embodiments, R¹ is

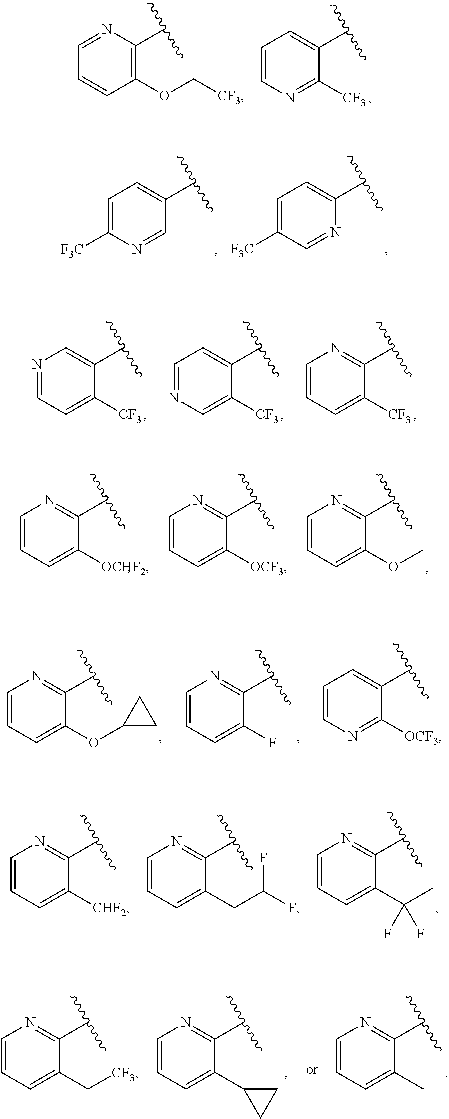

In certain embodiments, R¹ is

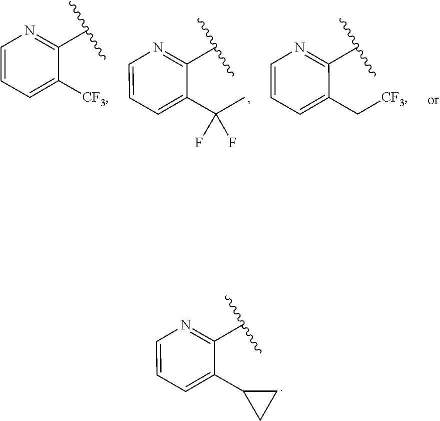

In certain embodiments, R¹ is

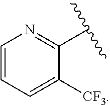 [SMILES: *c1ncccc1C(F)(F)F]

In certain embodiments, R¹ is

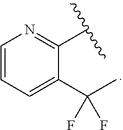 [SMILES: *c1ncccc1C(C)(F)F]

In certain embodiments, R¹ is

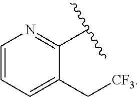 [SMILES: *c1ncccc1CC(F)(F)F]

In certain embodiments, R¹ is

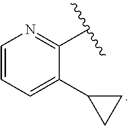 [SMILES: *c1ncccc1C1CC1]

In certain embodiments, R¹ is optionally substituted carbocyclyl. In certain embodiments, R¹ is optionally substituted carbocyclyl, wherein the carbocyclyl is fused with a heteroaryl ring. In certain embodiments, R¹ is optionally substituted C₈₋₁₄ carbocyclyl, wherein the carbocyclyl is fused with a heteroaryl ring. In certain embodiments, R¹ is

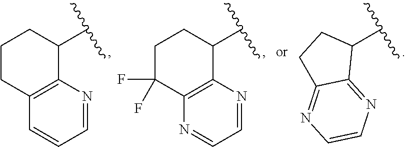

In certain embodiments, R¹ is

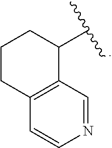 [SMILES: *C1CCCc2ccncc21]

In certain embodiments, R¹ is

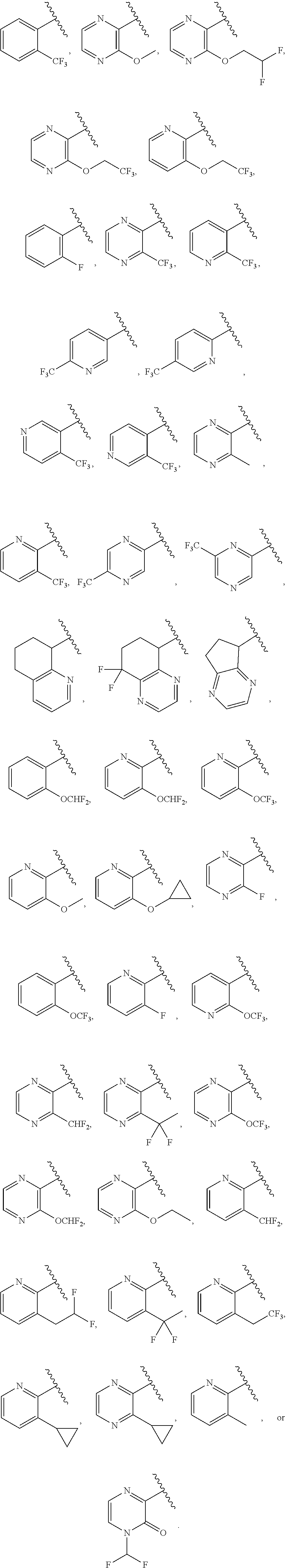

In certain embodiments, R¹ is

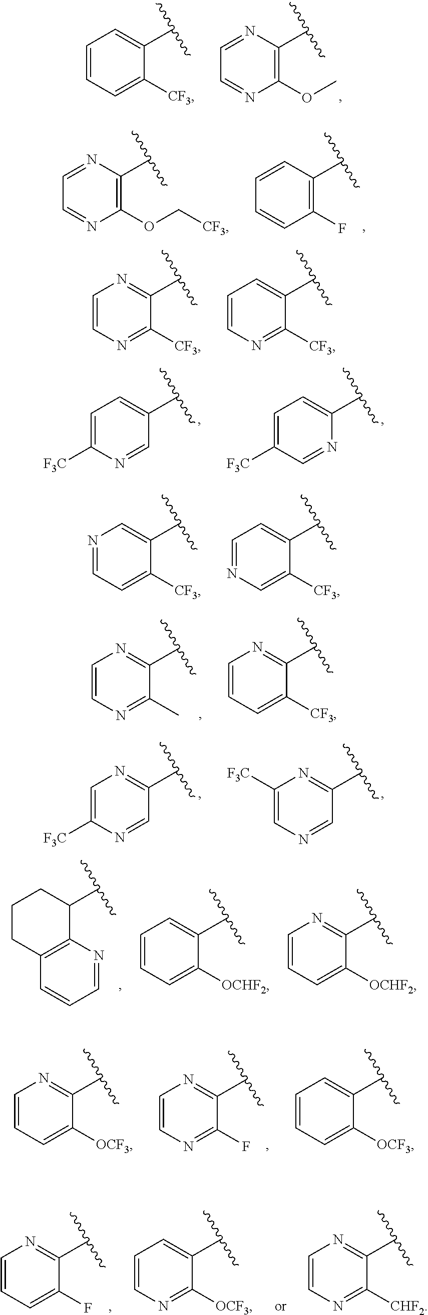

### R²

As described herein, R² is an optionally substituted carbocyclyl or optionally substituted heterocyclyl. In certain embodiments, R² is optionally substituted carbocyclyl. In certain embodiments, R² is optionally substituted cycloalkyl. In certain embodiments, R² is optionally substituted C₃₋₈ cycloalkyl. In certain embodiments, R² is optionally substituted C₃₋₆ cycloalkyl. In certain embodiments, R² is optionally substituted C₄₋₆ cycloalkyl. In certain embodiments, R² is optionally substituted C₅₋₆ cycloalkyl. In certain embodiments, R² is optionally substituted cyclopentyl. In certain embodiments, R² is optionally substituted cyclobutyl. In certain embodiments, R² is optionally substituted cyclohexyl.

In certain embodiments, R² is

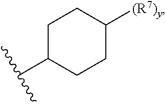

wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

In certain embodiments, R² is

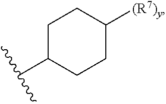

wherein each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; and y is 0, 1, or 2.

In certain embodiments, R² is

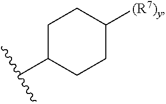

wherein each R⁷ is independently optionally substituted aryl or optionally substituted heteroaryl, or two instances of R⁷ together with the atoms to which they are attached form a substituted or unsubstituted heterocyclyl; and y is 0, 1, or 2.

In certain embodiments, R² is

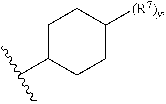

wherein each R⁷ is independently optionally substituted aryl or optionally substituted heteroaryl; and y is 1.

In certain embodiments, R² is

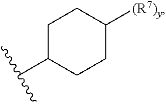

wherein each R⁷ is independently optionally substituted aryl, or two instances of R⁷ together with the atoms to which they are attached form a substituted or unsubstituted heterocyclyl; and y is 0, 1, or 2.

In certain embodiments, R² is

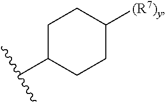

wherein each R⁷ is independently optionally substituted aryl, or two instances of R⁷ together with the atoms to which they are attached form a substituted or unsubstituted heterocyclyl; and y is 1 or 2.

In certain embodiments, R² is

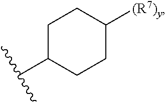

wherein each R⁷ is independently optionally substituted heteroaryl, or two instances of R⁷ together with the atoms to which they are attached form a substituted or unsubstituted heterocyclyl; and y is 1 or 2.

In certain embodiments, R² is

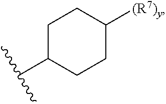

wherein R⁷ is optionally substituted aryl; and y is 1.

In certain embodiments, R² is

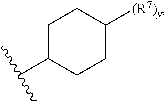

wherein R⁷ is substituted aryl; and y is 1.

In certain embodiments, R² is

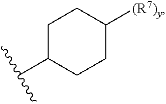

wherein R⁷ is substituted phenyl; and y is 1.

In certain embodiments, R² is

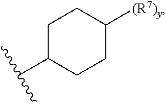

wherein R⁷ is optionally substituted heteroaryl; and y is 1.

In certain embodiments, R² is

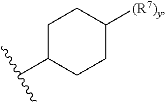

wherein R⁷ is substituted heteroaryl; and y is 1.

In certain embodiments, R² is

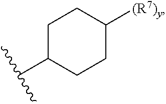

wherein R⁷ is substituted pyridine, substituted thiazole, substituted isothiazole, substituted substituted pyrazole, or substituted indazole; and y is 1.

In certain embodiments, R² is

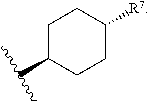

In certain embodiments, R² is

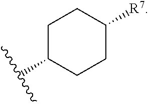

In certain embodiments, R² is

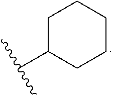 [SMILES: *C1CCCCC1]

In certain embodiments, R² is

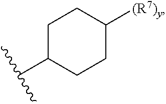

wherein two instances of R⁷ together with the atoms to which they are attached form a substituted or unsubstituted heterocyclyl; and y is 2.

In certain embodiments, R² is

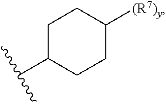

wherein two instances of R⁷ together with the atoms to which they are attached form a substituted heterocyclyl; and y is 2.

In certain embodiments, R² is

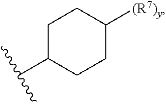

wherein two instances of R⁷ together with the atoms to which they are attached form a substituted 4-6 membered heterocyclyl; and y is 2.

In certain embodiments, R² is

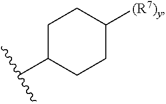

wherein two instances of R⁷ together with the atoms to which they are attached form a substituted azetidine; and y is 2.

In certain embodiments, R² is

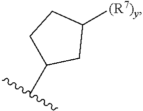

wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

In certain embodiments, R² is

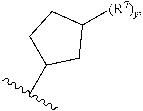

wherein R⁷ is optionally substituted aryl; and y is 1.

In certain embodiments, R² is

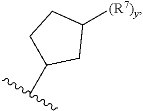

wherein R⁷ is substituted aryl; and y is 1.

In certain embodiments, R² is

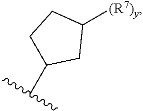

wherein R⁷ is substituted phenyl; and y is 1.

In certain embodiments, R² is

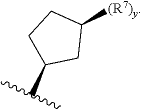

In certain embodiments, R² is

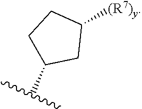

In certain embodiments, R² is

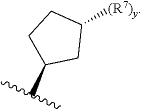

In certain embodiments, R² is

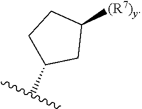

In certain embodiments, R² is

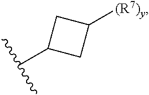

wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

In certain embodiments, R² is

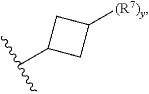

wherein R⁷ is optionally substituted aryl; and y is 1.

In certain embodiments, R² is

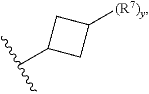

wherein R⁷ is substituted aryl; and y is 1.

In certain embodiments, R² is

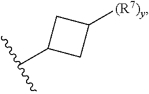

wherein R⁷ is substituted phenyl; and y is 1.

In certain embodiments, R² is

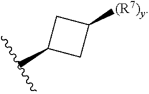

In certain embodiments, R² is

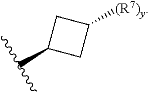

In certain embodiments, R² is

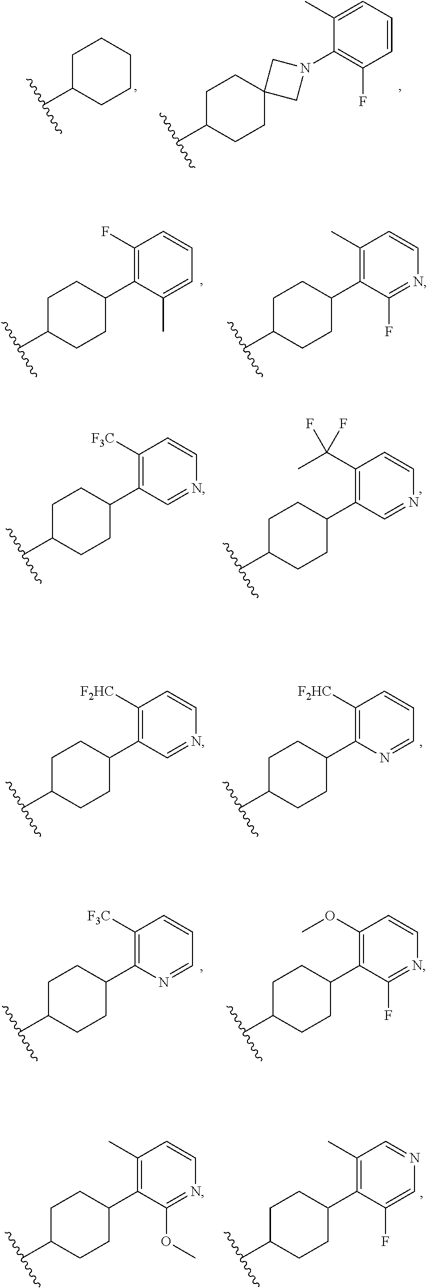

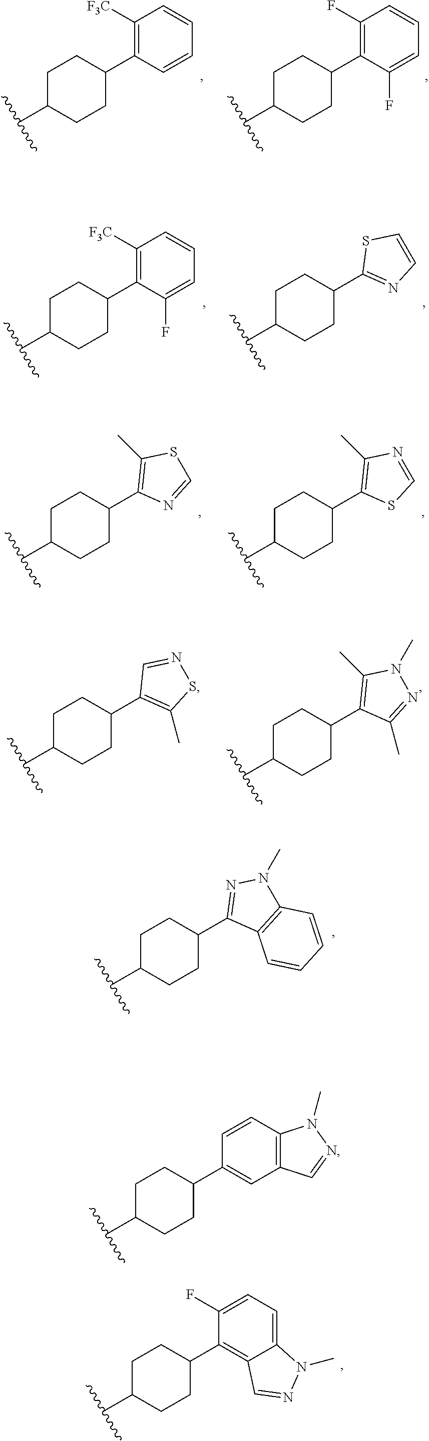

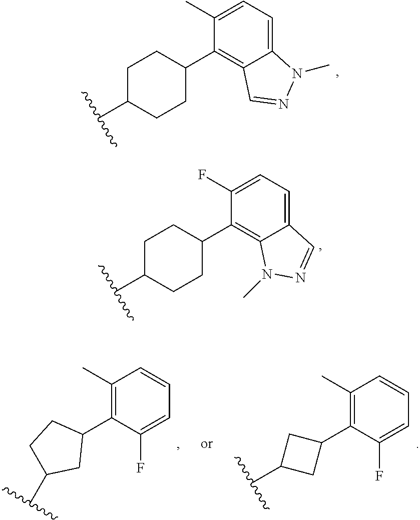 [SMILES: *C1CCC(c2c(C)ccc3c2cnn3C)CC1 ; *C1CCC(c2c(F)ccc3cnn(C)c23)CC1 ; *C1CCC(c2c(C)cccc2F)C1 ; *C1CC(c2c(C)cccc2F)C1]

In certain embodiments, R² is or

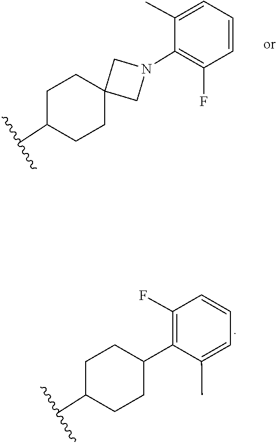 [SMILES: *C1CCC2(CC1)CN(c1c(C)cccc1F)C2 ; *C1CCC(c2c(C)cccc2F)CC1]

In certain embodiments, R² is

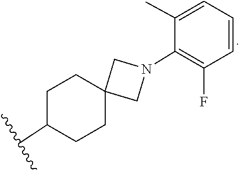 [SMILES: *C1CCC2(CC1)CN(c1c(C)cccc1F)C2]

In certain embodiments, R² is

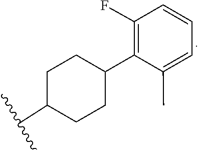 [SMILES: *C1CCC(c2c(C)cccc2F)CC1]

In certain embodiments, R² is

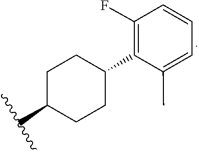 [SMILES: *[C@H]1CC[C@H](c2c(C)cccc2F)CC1]

In certain embodiments, R² is

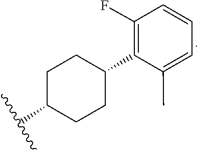 [SMILES: *[C@H]1CC[C@@H](c2c(C)cccc2F)CC1]

In certain embodiments, R² is optionally substituted heterocyclyl. In certain embodiments, R² is optionally substituted 4-7 membered heterocyclyl comprising 1 or 2 nitrogen atoms in the ring. In certain embodiments, R² is optionally substituted azetidine, pyrrolidine, piperidine, or piperazine. In certain embodiments, R² is optionally substituted azetidine. In certain embodiments, R² is optionally substituted pyrrolidine. In certain embodiments, R² is optionally substituted piperidine. In certain embodiments, R² is optionally substituted piperazine.

In certain embodiments, R² is

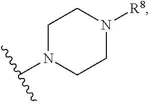

wherein R⁸ is optionally substituted alkyl, optionally substituted aryl, optionally substituted heteroaryl, or a nitrogen protecting group.

In certain embodiments, R² is

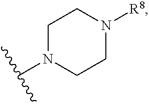

wherein R⁸ is a nitrogen protecting group (e.g., BOC).

In certain embodiments, R² is

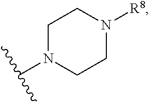

wherein R⁸ is optionally substituted aryl, optionally substituted heteroaryl, or a nitrogen protecting group. In certain embodiments, R² is

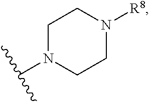

wherein R⁸ is aryl substituted with at least one instance of —Oalkyl, —Ohaloalkyl, alkyl, halogen, or haloalkyl, heteroaryl substituted with at least one instance of —Ohaloalkyl, alkyl, halogen, or haloalkyl, or a nitrogen protecting group. In certain embodiments, R² is

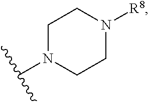

wherein R⁸ is aryl substituted with at least one instance of —F, —CH₃, CF₃, —CHF₂, —OCH₃, —OCF₃, or —OCHF₂, heteroaryl substituted with at least one instance of —F, —CH₃, CF₃, —CHF₂, —OCH₃, —OCF₃, or —OCHF₂, or a nitrogen protecting group.

In certain embodiments, R² is

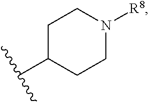

wherein R⁸ is optionally substituted alkyl, optionally substituted aryl, optionally substituted heteroaryl, or a nitrogen protecting group.

In certain embodiments, R² is

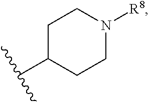

wherein R⁸ is a nitrogen protecting group (e.g., BOC).

In certain embodiments, R² is

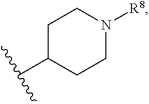

wherein R⁸ is optionally substituted aryl, optionally substituted heteroaryl, or a nitrogen protecting group. In certain embodiments, R² is

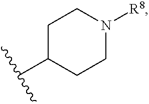

wherein R⁸ is aryl substituted with at least one instance of-Oalkyl, —Ohaloalkyl, alkyl, halogen, or haloalkyl, heteroaryl substituted with at least one instance of —Ohaloalkyl, alkyl, halogen, or haloalkyl, or a nitrogen protecting group. In certain embodiments, R² is

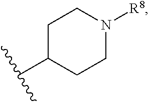

wherein R⁸ is aryl substituted with at least one instance of —F, —CH₃, CF₃, —CHF₂, —OCH₃, —OCF₃, or —OCHF₂, heteroaryl substituted with at least one instance of —F, —CH₃, CF₃, —CHF₂, —OCH₃, —OCF₃, or —OCHF₂, or a nitrogen protecting group.

In certain embodiments, R² is

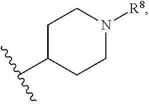

wherein R⁸ is optionally substituted aryl or optionally substituted heteroaryl. In certain embodiments, R² is

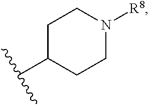

wherein R⁸ is aryl substituted with at least one instance of-Oalkyl, —Ohaloalkyl, alkyl, halogen, or haloalkyl; or heteroaryl substituted with at least one instance of —Ohaloalkyl, alkyl, halogen, or haloalkyl. In certain embodiments, R² is

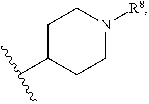

wherein R⁸ is aryl substituted with at least one instance of —F, —CH₃, CF₃, —CHF₂, —OCH₃, —OCF₃, or —OCHF₂; or heteroaryl substituted with at least one instance of —F, —CH₃, CF₃, —CHF₂, —OCH₃, —OCF₃, or —OCHF₂.

In certain embodiments, R² is

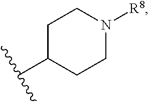

wherein R⁸ is optionally substituted aryl. In certain embodiments, R² is

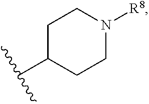

wherein R⁸ is aryl substituted with at least one instance of-Oalkyl, —Ohaloalkyl, alkyl, halogen, or haloalkyl. In certain embodiments, R² is

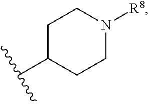

wherein R⁸ is aryl substituted with at least one instance of —F, —CH₃, CF₃, —CHF₂, —OCH₃, —OCF₃, or —OCHF₂.

In certain embodiments, R² is

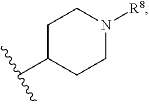

wherein R⁸ is optionally substituted phenyl. In certain embodiments, R² is

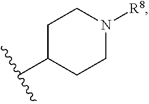

wherein R⁸ is substituted phenyl. In certain embodiments, R² is

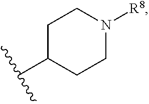

wherein R⁸ is phenyl substituted with at least one instance of-Oalkyl, —Ohaloalkyl, alkyl, halogen, or haloalkyl. In certain embodiments, R² is

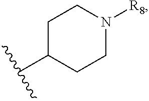

wherein R⁸ is phenyl substituted with at least one instance of —F, —CH₃, CF₃, —CHF₂, —OCH₃, —OCF₃, or —OCHF₂.

In certain embodiments, R² is

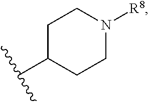

wherein R⁸ is optionally substituted heteroaryl. In certain embodiments, R² is

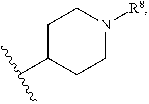

wherein R⁸ is heteroaryl substituted with at least one instance of-Oalkyl, —Ohaloalkyl, alkyl, halogen, or haloalkyl. In certain embodiments, R² is

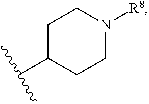

wherein R⁸ is heteroaryl substituted with at least one instance of —F, —CH₃, CF₃, —CHF₂, —OCH₃, —OCF₃, or —OCHF₂.

In certain embodiments, R² is

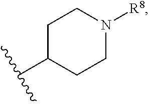

wherein R⁸ is optionally substituted pyrazine, optionally substituted pyridine, optionally substituted pyridazine, optionally substituted pyrimidine, optionally substituted thiazole, optionally substituted thiadiazole, or optionally substituted indazole. In certain embodiments, R² is

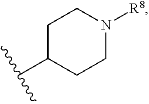

wherein R⁸ is substituted pyrazine, substituted pyridine, substituted pyridazine, substituted pyrimidine, substituted thiazole, unsubstituted thiadiazole, or substituted indazole. In certain embodiments, R² is

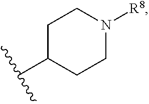

wherein R⁸ is pyrazine, pyridine, pyridazine, pyrimidine, thiazole, thiadiazole, or indazole, each of which is optionally substituted with at least one instance of-Oalkyl, —Ohaloalkyl, alkyl, halogen, or haloalkyl. In certain embodiments, R² is

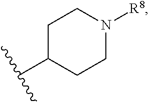

wherein R⁸ is pyrazine, pyridine, pyridazine, pyrimidine, thiazole, thiadiazole, or indazole, each of which is optionally substituted with at least one instance of —F, —CH₃, CF₃, —CHF₂, —OCH₃, —OCF₃, or —OCHF₂.

In certain embodiments, R² is

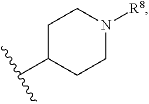

wherein R⁸ is optionally substituted pyrazine, optionally substituted pyridine, optionally substituted pyridazine, or optionally substituted pyrimidine. In certain embodiments, R² is

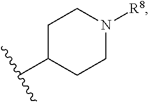

wherein R⁸ is substituted pyrazine, substituted pyridine, substituted pyridazine, or substituted pyrimidine. In certain embodiments, R² is

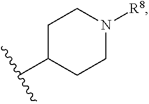

wherein R⁸ is pyrazine, pyridine, pyridazine, or pyrimidine, each of which is substituted with at least one instance of-Oalkyl, —Ohaloalkyl, alkyl, halogen, or haloalkyl. In certain embodiments, R² is

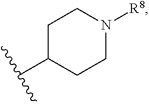

wherein R⁸ is pyrazine, pyridine, pyridazine, or pyrimidine, each of which is substituted with at least one instance of —F, —CH₃, CF₃, —CHF₂, —OCH₃, —OCF₃, or —OCHF₂.

In certain embodiments, R² is

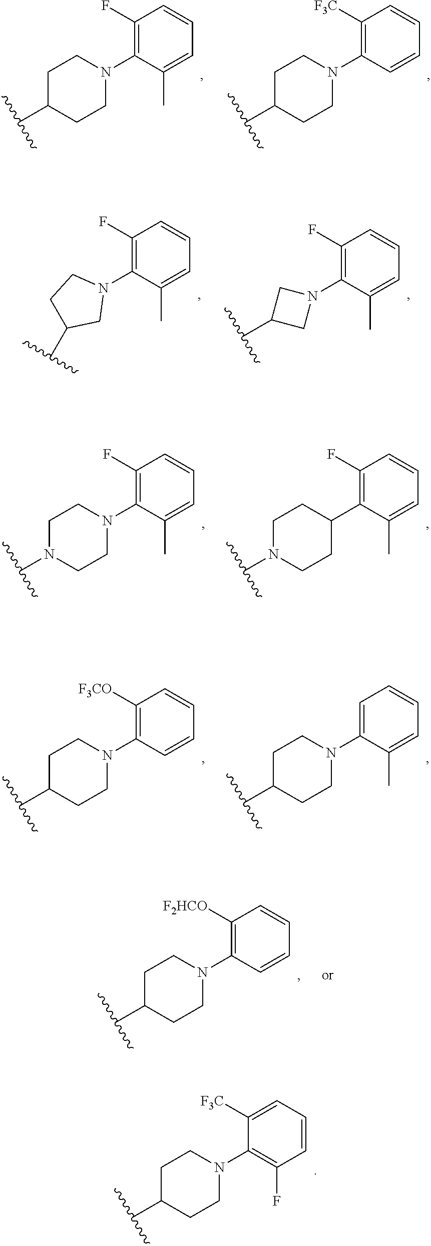

In certain embodiments, R² is

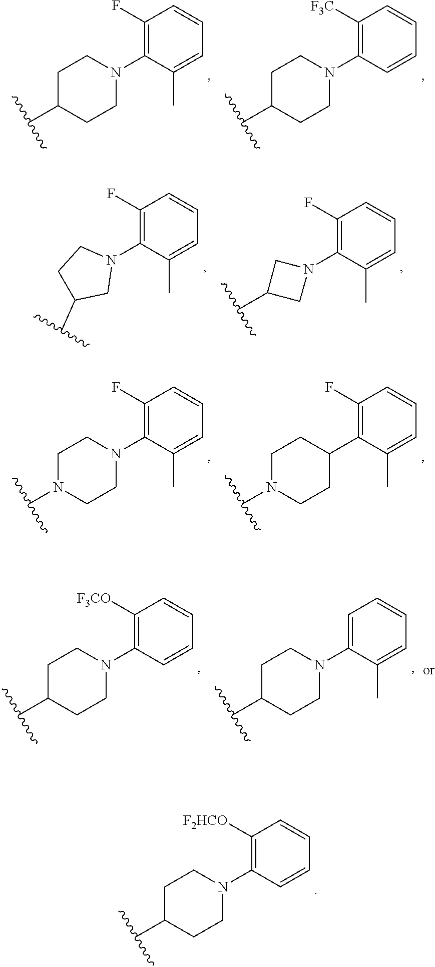

In certain embodiments, R² is

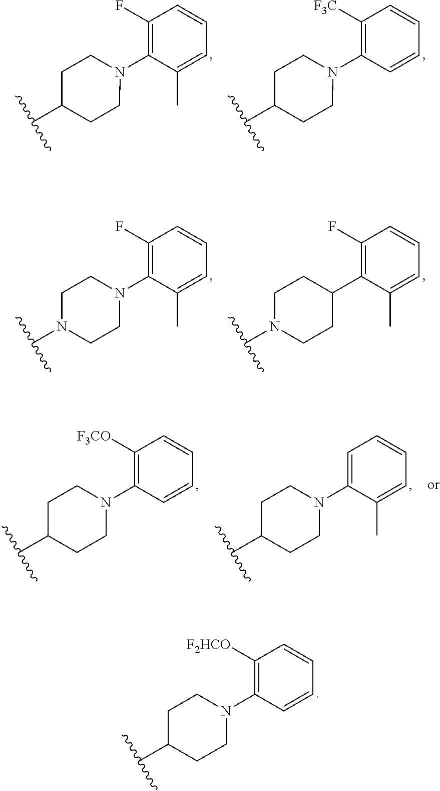

In certain embodiments, R² is

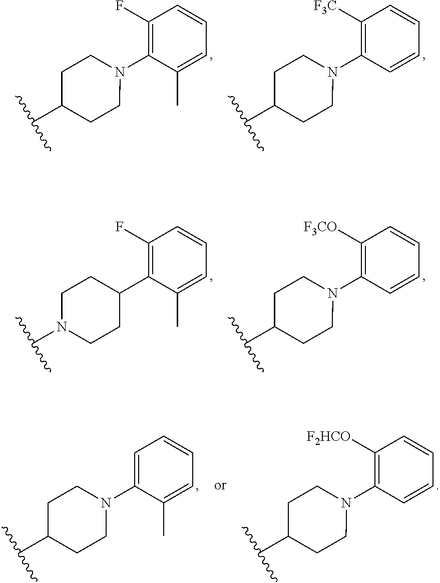 [SMILES: *C1CCN(c2c(C)cccc2F)CC1 ; *C1CCN(c2ccccc2C(F)(F)F)CC1 ; *N1CCC(c2c(C)cccc2F)CC1 ; *C1CCN(c2ccccc2OC(F)(F)F)CC1 ; *C1CCN(c2ccccc2C)CC1 ; *C1CCN(c2ccccc2OC(F)F)CC1]

In certain embodiments, R² is

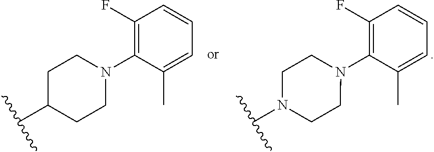 [SMILES: *C1CCN(c2c(C)cccc2F)CC1 ; *N1CCN(c2c(C)cccc2F)CC1]

In certain embodiments, R² is

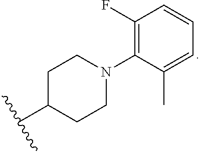 [SMILES: *C1CCN(c2c(C)cccc2F)CC1]

In certain embodiments, R² is

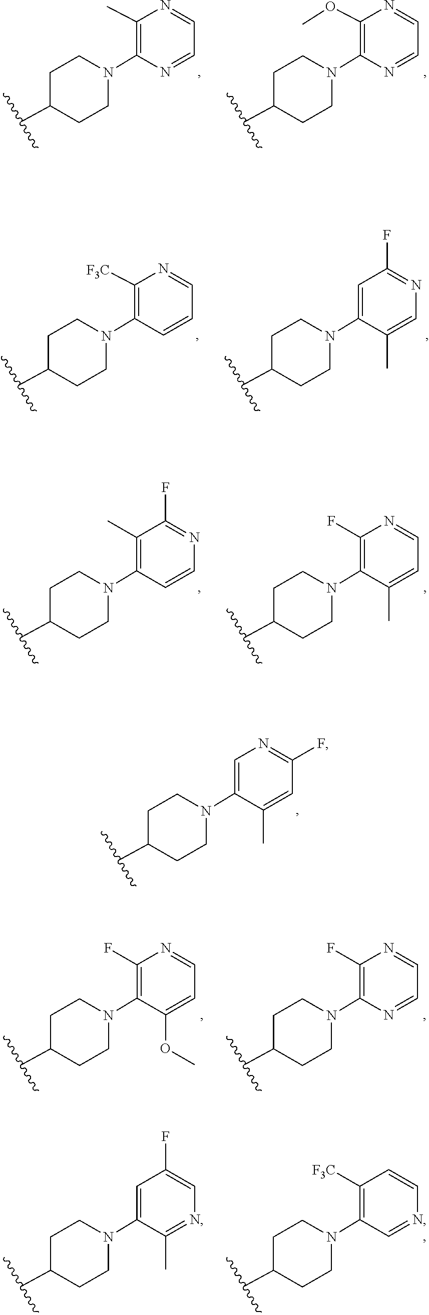

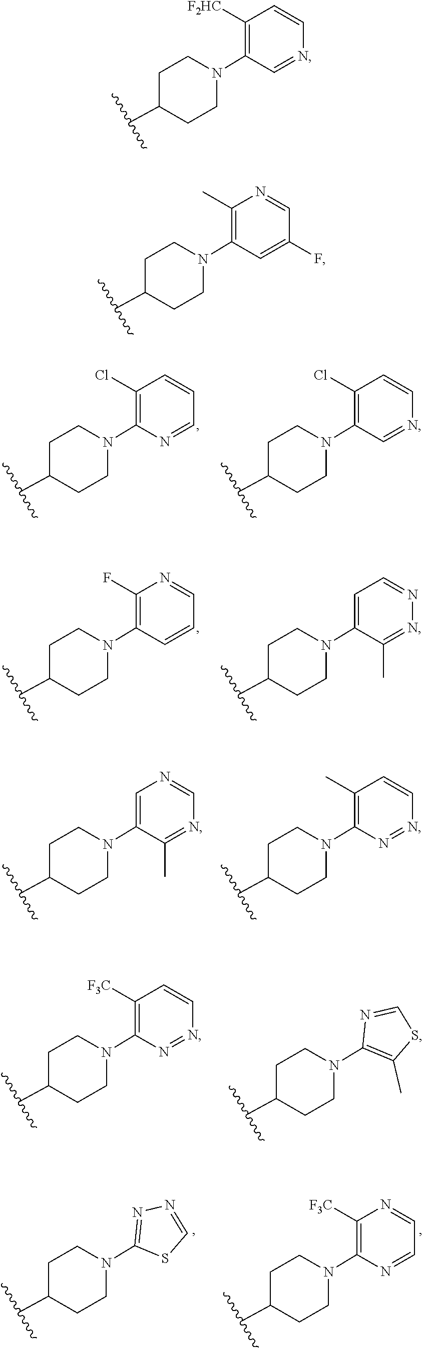

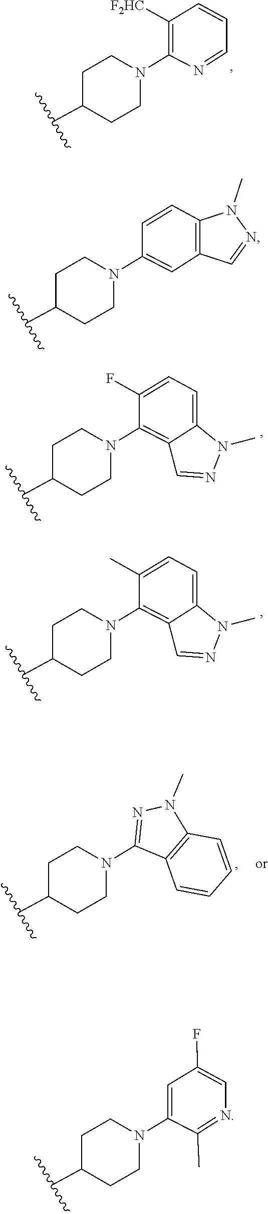 [SMILES: *C1CCN(c2ncccc2C(F)F)CC1 ; *C1CCN(c2ccc3c(cnn3C)c2)CC1 ; *C1CCN(c2c(F)ccc3c2cnn3C)CC1 ; *C1CCN(c2c(C)ccc3c2cnn3C)CC1 ; *C1CCN(c2nn(C)c3ccccc23)CC1 ; *C1CCN(c2cc(F)cnc2C)CC1]

In certain embodiments, R² is

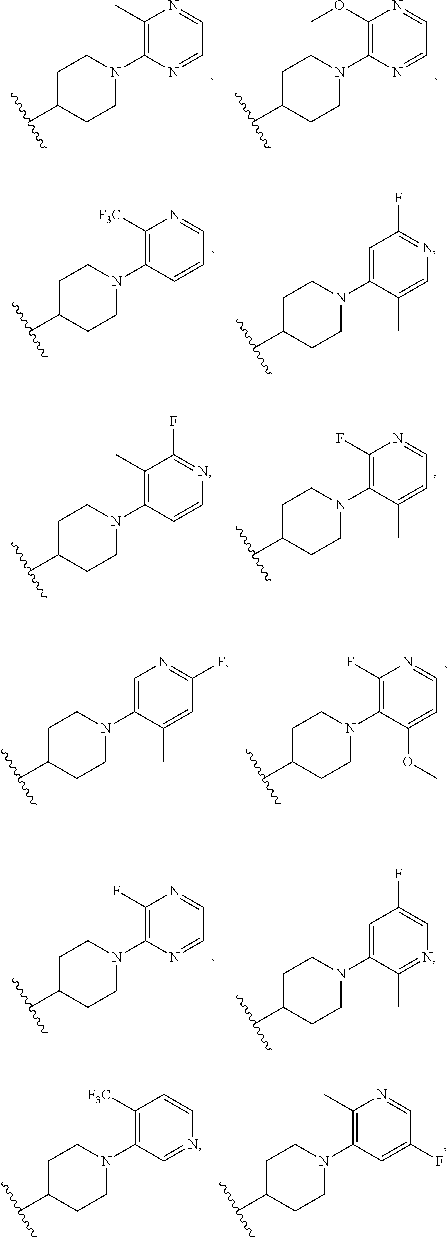

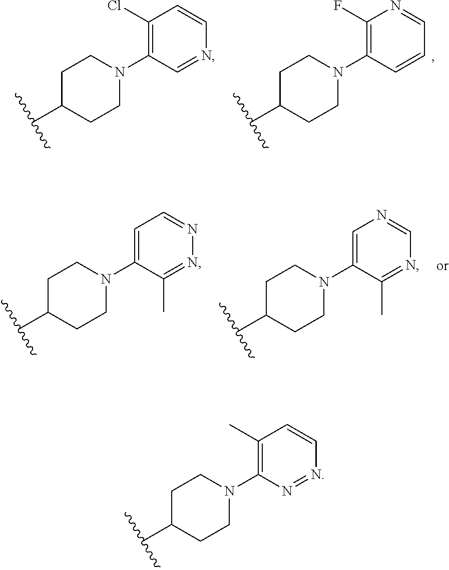 [SMILES: *C1CCN(c2cnccc2Cl)CC1 ; *C1CCN(c2cccnc2F)CC1 ; *C1CCN(c2ccnnc2C)CC1 ; *C1CCN(c2cncnc2C)CC1 ; *C1CCN(c2nnccc2C)CC1]

In certain embodiments, R² is

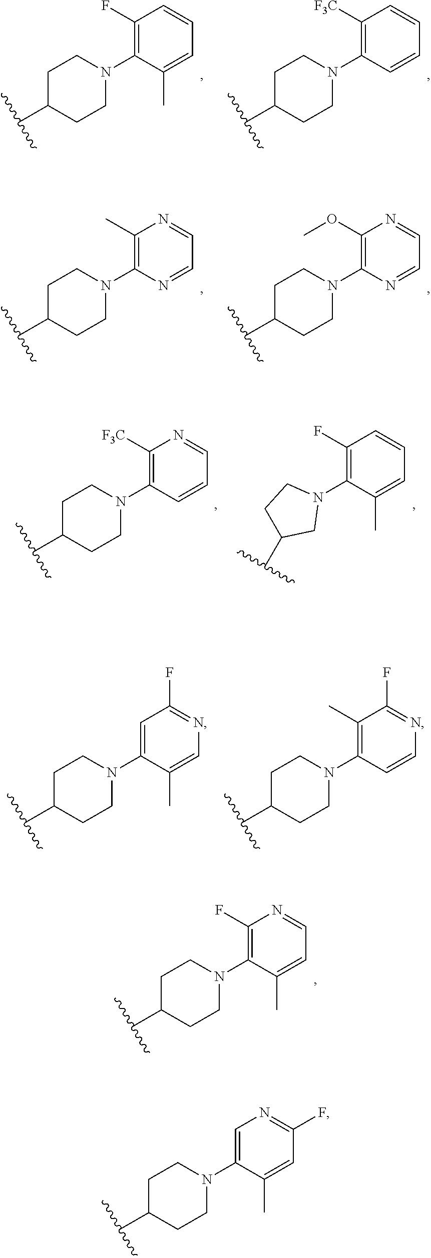

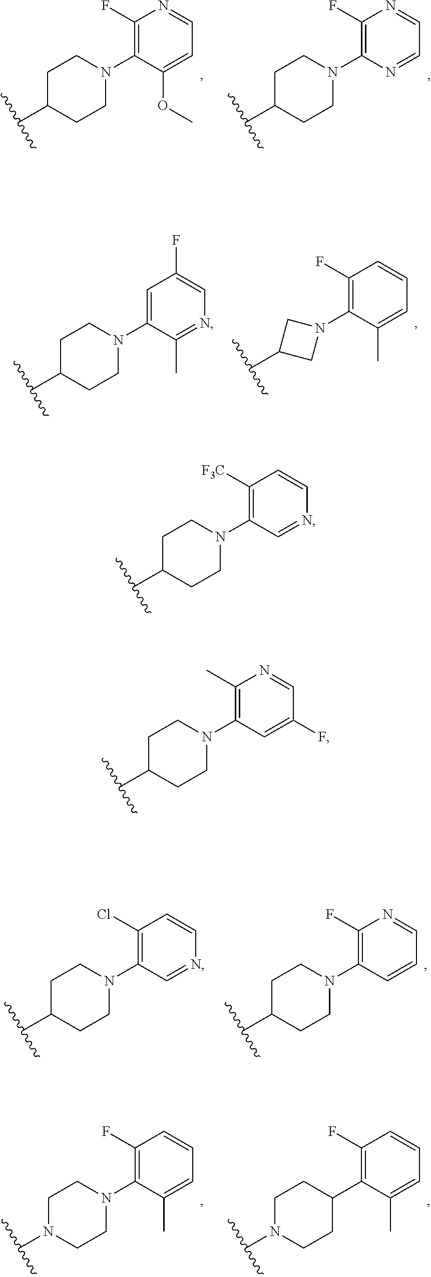

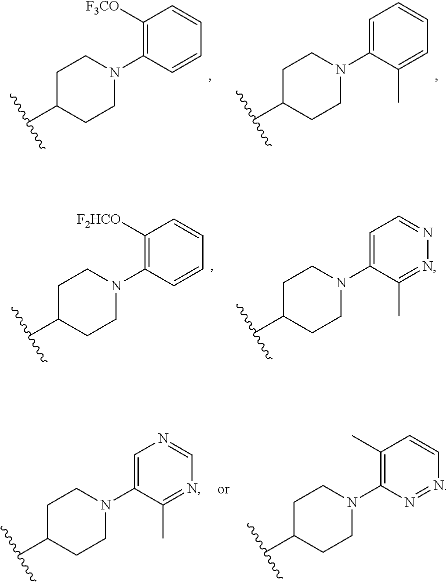 [SMILES: *C1CCN(c2ccccc2OC(F)(F)F)CC1 ; *C1CCN(c2ccccc2C)CC1 ; *C1CCN(c2ccccc2OC(F)F)CC1 ; *C1CCN(c2ccnnc2C)CC1 ; *C1CCN(c2cncnc2C)CC1 ; *C1CCN(c2nnccc2C)CC1]

In certain embodiments, R² is,

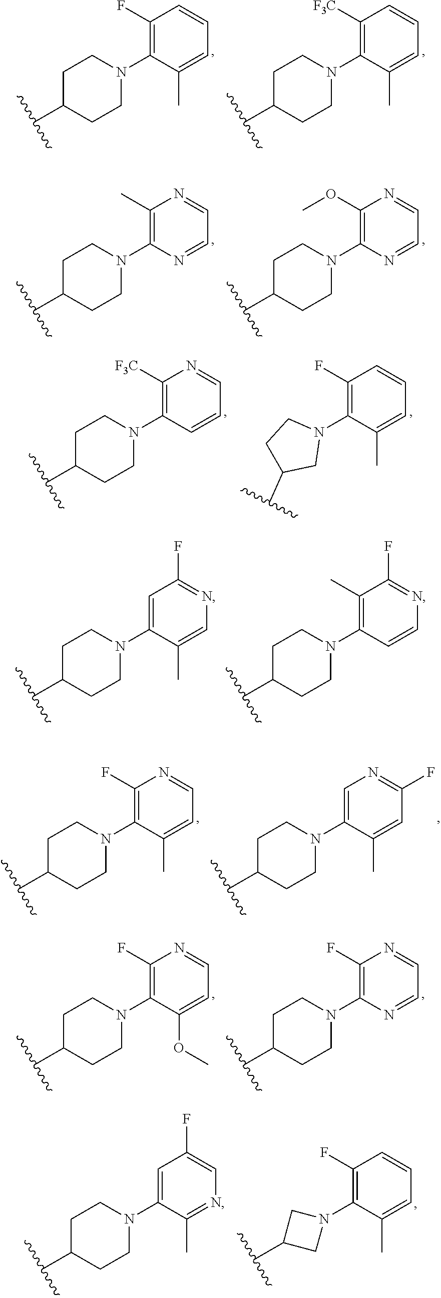

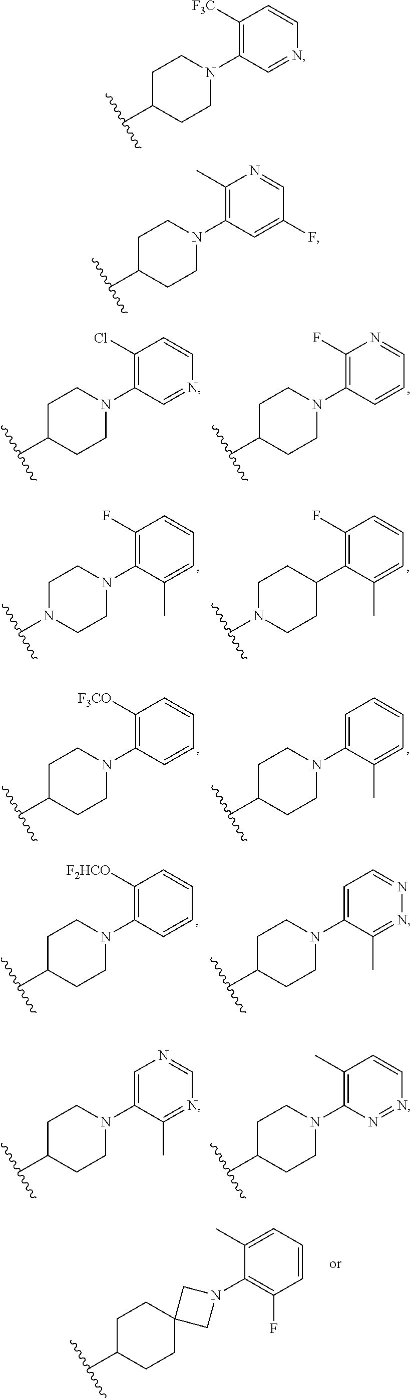

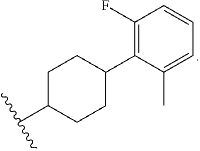 [SMILES: *C1CCC(c2c(C)cccc2F)CC1]

In certain embodiments, R² is

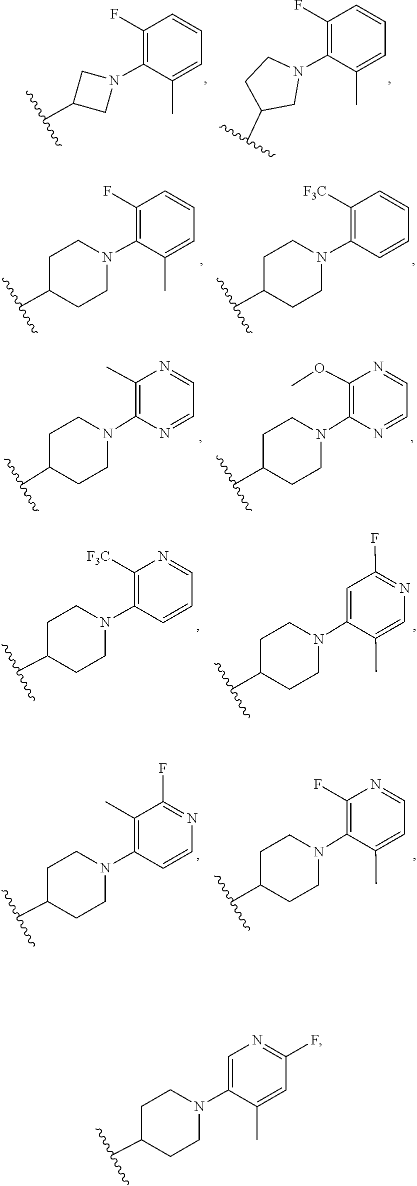

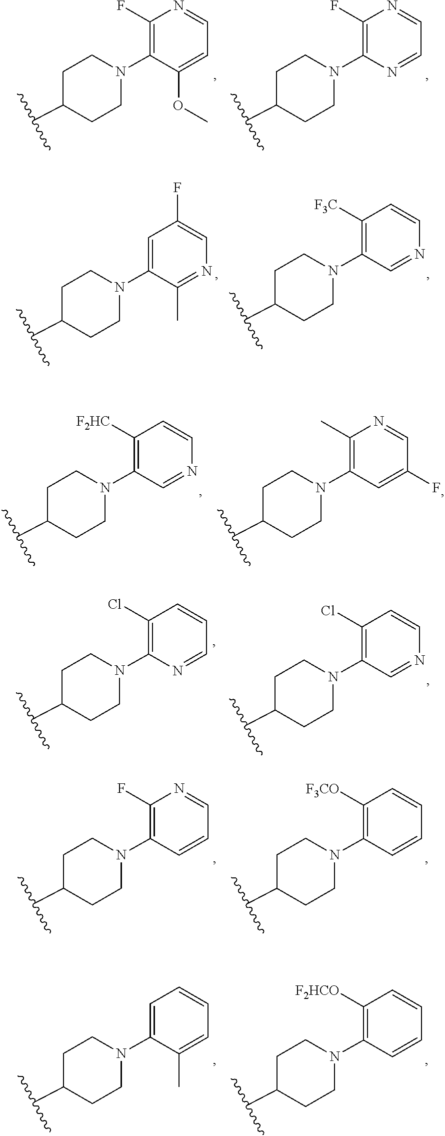

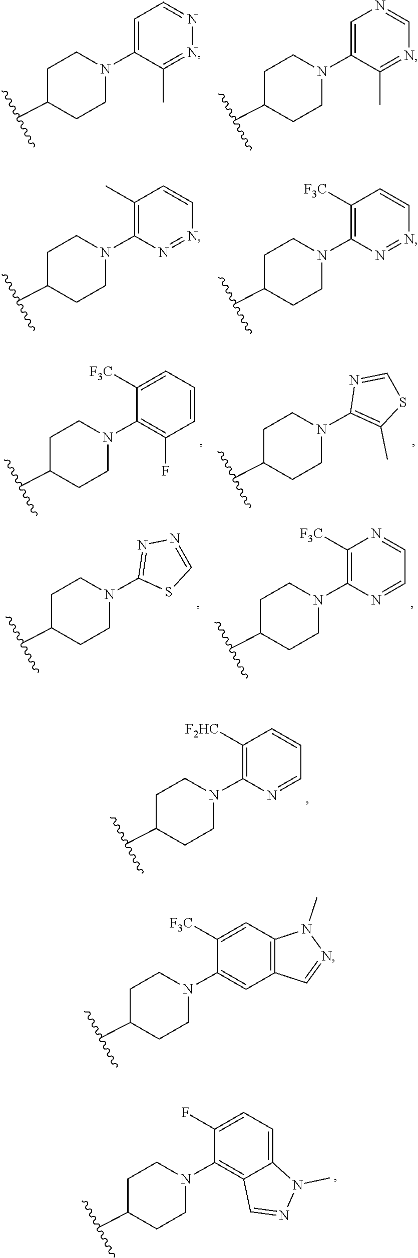

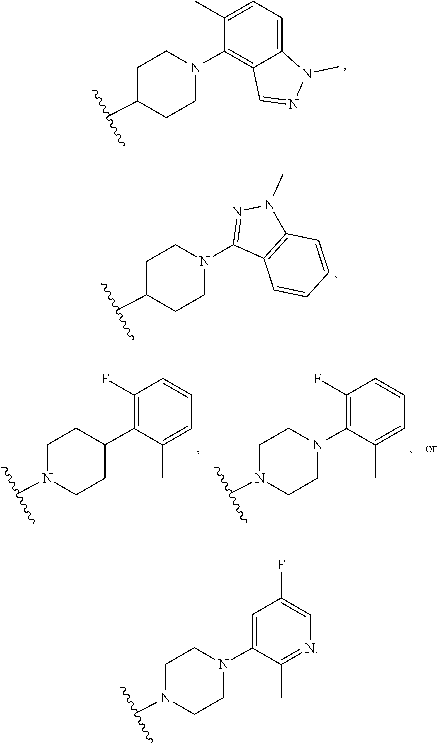 [SMILES: *C1CCN(c2c(C)ccc3c2cnn3C)CC1 ; *C1CCN(c2nn(C)c3ccccc23)CC1 ; *N1CCC(c2c(C)cccc2F)CC1 ; *N1CCN(c2c(C)cccc2F)CC1 ; *N1CCN(c2cc(F)cnc2C)CC1]

B¹ and B²

As described herein, B¹ is —C(R⁴)ₜ— or —N—, wherein t is 0 or 1 as valency permits; and B² is ═C(R⁴)—, —N—, or —C(O)—. In certain embodiments, t is 0. In certain embodiments, t is 1.

In certain embodiments, B¹ is —CR⁴—. In certain embodiments, B¹ is ═C— or —N—. In certain embodiments, B¹ is ═C—. In certain embodiments, B¹ is —N—.

In certain embodiments, B² is ═C(R⁴)—, —N—, or —C(O)—. In certain embodiments, B² is —N—. In certain embodiments, B² is —C(O)—. In certain embodiments, B² is ═C(R⁴)—. In certain embodiments, B² is ═C(H)—. In certain embodiments, B² is ═C(CH₃)—.

As described herein, each R⁴ is independently hydrogen, halogen, optionally substituted alkyl, optionally substituted alkenyl, optionally substituted alkynyl, —OR^{A}, —N(R^{A})₂, —SR^{A}, or —CN. In certain embodiments, each R⁴ is independently hydrogen or optionally substituted alkyl. In certain embodiments, each R⁴ is hydrogen. In certain embodiments, each R⁴ is independently optionally substituted alkyl. In certain embodiments, each R⁴ is independently unsubstituted alkyl. In certain embodiments, each R⁴ is independently unsubstituted C₁₋₄ alkyl. In certain embodiments, each R⁴ is independently —CH₃.

A¹, A², A³, and A⁴

As described herein, A¹, A², A³, and A⁴ are each independently ═C(R³)— or ═N—; and each R³ is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A}. In certain embodiments, each R³ is independently hydrogen, optionally substituted alkyl, or —OR^{A}. In certain embodiments, each R³ is independently hydrogen, unsubstituted alkyl, or —OR^{A}. In certain embodiments, each R³ is independently hydrogen, unsubstituted alkyl, or —OR^{A}; and R^{A} is unsubstituted alkyl. In certain embodiments, each R³ is independently hydrogen, —CH₃, —OCH₃, or —OCH₂CH₃. In certain embodiments, each R³ is hydrogen. In certain embodiments, each R³ is independently hydrogen or unsubstituted alkyl. In certain embodiments, each R³ is independently hydrogen or —OR^{A}. In certain embodiments, each R³ is independently hydrogen or —OR^{A}; and R^{A} is unsubstituted alkyl. In certain embodiments, each R³ is independently hydrogen, —OCH₃, or —OCH₂CH₃. In certain embodiments, each R³ is independently hydrogen or —OCH₃. In certain embodiments, each R³ is independently hydrogen or —CH₃. In certain embodiments, each R³ is independently hydrogen, —CH₃, —CH₂CH₃, or —CH₂CH₂CH₃. In certain embodiments, each R³ is independently hydrogen, or —CH₂CH₃. In certain embodiments, each R³ is independently hydrogen or —CH₂CH₂CH₃.

In certain embodiments, each R³ is independently hydrogen or optionally substituted alkyl. In certain embodiments, each R³ is independently hydrogen or substituted alkyl. In certain embodiments, each R³ is independently hydrogen or haloalkyl. In certain embodiments, each R³ is independently hydrogen, —CHF₂, —CF₃, —CH₂CF₃, —CF₂CH₃, or —CH₂CF₂H. In certain embodiments, each R³ is independently hydrogen or —CHF₂. In certain embodiments, each R³ is independently hydrogen or —CF₃. In certain embodiments, each R³ is independently hydrogen or —CH₂CF₃. In certain embodiments, each R³ is independently hydrogen or —CF₂CH₃. In certain embodiments, each R³ is independently hydrogen or —CH₂CF₂H.

In certain embodiments, each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H.

In certain embodiments, A¹ is ═N—. In certain embodiments, A¹ is ═C(R³)—. In certain embodiments, A¹ is ═C(R³)—; wherein R³ is independently hydrogen, optionally substituted alkyl, or —OR^{A}. In certain embodiments, A¹ is ═C(R³)—; wherein R³ is independently hydrogen, unsubstituted alkyl, or —OR^{A}; and R^{A} is unsubstituted alkyl. In certain embodiments, A¹ is ═C(R³)—; wherein R³ is independently hydrogen, —CH₃, —OCH₃, or —OCH₂CH₃. In certain embodiments, A¹ is ═C(R³)—; wherein R³ is hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H.

In certain embodiments, A² is ═N—. In certain embodiments, A² is ═C(R³)—. In certain embodiments, A² is ═C(R³)—; wherein R³ is independently hydrogen, optionally substituted alkyl, or —OR^{A}. In certain embodiments, A² is ═C(R³)—; wherein R³ is independently hydrogen, unsubstituted alkyl, or —OR^{A}; and R^{A} is unsubstituted alkyl. In certain embodiments, A² is ═C(R³)—; wherein R³ is independently hydrogen, —CH₃, —OCH₃, or —OCH₂CH₃. In certain embodiments, A² is ═C(R³)—; wherein R³ is hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H.

In certain embodiments, A³ is ═N—. In certain embodiments, A³ is ═C(R³)—. In certain embodiments, A³ is ═C(R³)—; wherein R³ is independently hydrogen, optionally substituted alkyl, or —OR^{A}. In certain embodiments, A³ is ═C(R³)—; wherein R³ is independently hydrogen, unsubstituted alkyl, or —OR^{A}; and R^{A} is unsubstituted alkyl. In certain embodiments, A³ is ═C(R³)—; wherein R³ is independently hydrogen, —CH₃, —OCH₃, or —OCH₂CH₃. In certain embodiments, A³ is ═C(R³)—; wherein R³ is hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H.

In certain embodiments, A⁴ is ═N—. In certain embodiments, A⁴ is ═C(R³)—. In certain embodiments, A⁴ is ═C(R³)—; wherein R³ is independently hydrogen, optionally substituted alkyl, or —OR^{A}. In certain embodiments, A⁴ is ═C(R³)—; wherein R³ is independently hydrogen, unsubstituted alkyl, or —OR^{A}; and R^{A} is unsubstituted alkyl. In certain embodiments, A⁴ is ═C(R³)—; wherein R³ is independently hydrogen, —CH₃, —OCH₃, or —OCH₂CH₃. In certain embodiments, A⁴ is ═C(R³)—; wherein R³ is hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H.

In certain embodiments, no more than two of A¹, A², A³, and A⁴ are ═N—. In certain embodiments, no more than one of A¹, A², A³, and A⁴ is ═N—. In certain embodiments, A¹ and A⁴ are ═N—; and A² and A³ are ═C(R³)—. In certain embodiments, A¹ and A³ are ═N—; and A² and A⁴ are ═C(R³)—. In certain embodiments, A² and A⁴ are ═N—; and A¹ and A³ are ═C(R³)—. In certain embodiments, A¹, A², and A³ are ═C(R³)—; and A⁴ is ═N—. In certain embodiments, A², A³, and A⁴ are ═C(R³)—; and A¹ is ═N—. In certain embodiments, A¹, A², A³, and A⁴ are ═C(R³)—.

In certain embodiments, A¹ and A⁴ are ═N—; and A² and A³ are ═C(R³)—, wherein each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H. In certain embodiments, A¹ and A³ are ═N—; and A² and A⁴ are ═C(R³)—, wherein each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H. In certain embodiments, A² and A⁴ are ═N—; and A¹ and A³ are ═C(R³)—, wherein each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H. In certain embodiments, A¹, A², and A³ are ═C(R³)—; and A⁴ is ═N—, wherein each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H. In certain embodiments, A², A³, and A⁴ are ═C(R³)—; and A¹ is ═N—, wherein each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H. In certain embodiments, A¹, A², A³, and A⁴ are ═C(R³)—, wherein each R³ is independently hydrogen, —OCH₃, —OCH₂CH₃, —CH₃, —CH₂CH₃, —CH₂CH₂CH₃. —CHF₂, —CF₃, —CH₂CF₃, —CF₂CH₃, or —CH₂CF₂H.

m and n

As described herein, each occurrence of R^{A} is, independently, hydrogen, optionally substituted acyl, optionally substituted alkyl, optionally substituted alkenyl, optionally substituted alkynyl, optionally substituted heteroaliphatic, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom.

In certain embodiments, each occurrence of R^{A} is, independently, hydrogen, optionally substituted acyl, optionally substituted alkyl, an oxygen protecting group when attached to an oxygen atom, or a nitrogen protecting group when attached to a nitrogen atom.

In certain embodiments, each occurrence of R^{A} is, independently, hydrogen, or optionally substituted alkyl. In certain embodiments, each occurrence of R^{A} is, independently, hydrogen or unsubstituted alkyl. In certain embodiments, R^{A} is hydrogen.

In certain embodiments, each occurrence of R^{A} is, independently, haloalkyl or unsubstituted alkyl. In certain embodiments, each occurrence of R^{A} is, independently, haloalkyl. In certain embodiments, each occurrence of R^{A} is, independently, unsubstituted alkyl. In certain embodiments, each R^{A} is independently C₁₋₄ haloalkyl or unsubstituted C₁₋₄ alkyl. In certain embodiments, each R^{A} is independently C₁₋₄ haloalkyl. In certain embodiments, each R^{A} is independently unsubstituted C₁₋₄ alkyl. In certain embodiments, each R^{A} is independently —CHF₂, —CF₃, —CH₂CF₃, —CH₂CH₃, or —CH₃. In certain embodiments, each R^{A} is independently —CF₃. In certain embodiments, each R^{A} is independently —CH₃.

### Certain Embodiments

In certain embodiments, the compound of Formula (I) is of Formula (I-a):

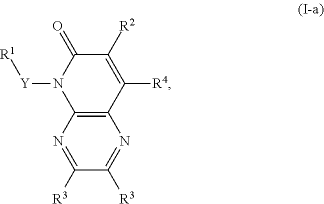

or a pharmaceutically acceptable salt, co-crystal, tautomer, stereoisomer, solvate, hydrate, polymorph, isotopically enriched derivative, or prodrug thereof; wherein R¹, Y, R², R³, and R⁴ are as defined herein.

In certain embodiments, the compound of Formula (I) is of Formula (I-a-1):

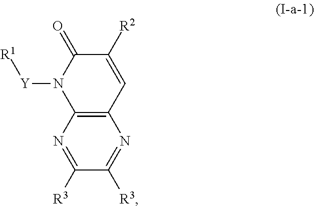

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-a-2):

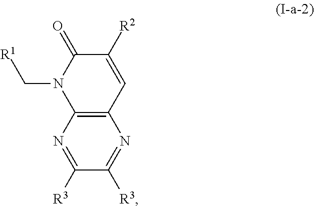

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-a-3):

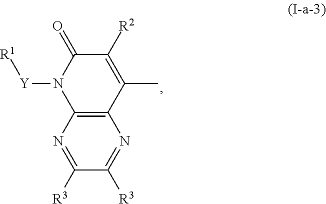

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-a-4):

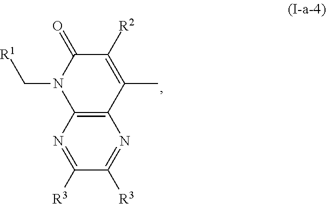

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-a-5):

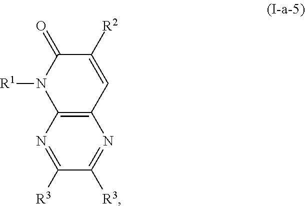

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-a-6):

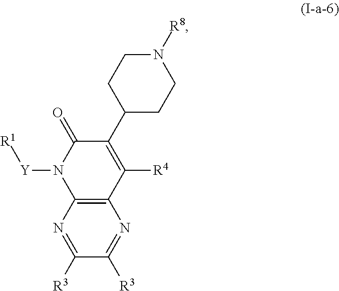

or a pharmaceutically acceptable salt, co-crystal, tautomer, stereoisomer, solvate, hydrate, polymorph, isotopically enriched derivative, or prodrug thereof; wherein R⁸ is optionally substituted alkyl, optionally substituted aryl, optionally substituted heteroaryl, or a nitrogen protecting group; and Y, R¹, R³, and R⁴ are as defined herein.

In certain embodiments, the compound of Formula (I) is of Formula (I-a-7):

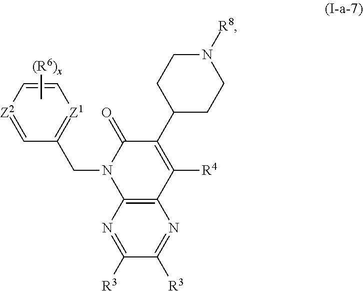

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; R⁸ is optionally substituted alkyl, optionally substituted aryl, optionally substituted heteroaryl, or a nitrogen protecting group; and R³ and R⁴ are as defined herein.

In certain embodiments, Z¹ and Z² are each independently CH, CR⁶, or N; each R⁶ is independently halogen, optionally substituted alkyl, or —OR^{A}; x is 0, 1, 2, or 3; and R⁸ is optionally substituted alkyl, optionally substituted aryl, optionally substituted heteroaryl, or a nitrogen protecting group.

In certain embodiments, the compound of Formula (I) is of Formula (I-a-8):

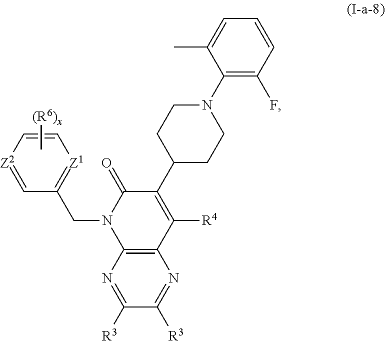

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, Z¹ and Z² are each independently CH, CR⁶, or N; each R⁶ is independently halogen, optionally substituted alkyl, or —OR^{A}; and x is 0, 1, 2, or 3.

In certain embodiments, the compound of Formula (I) is of Formula (I-a-9):

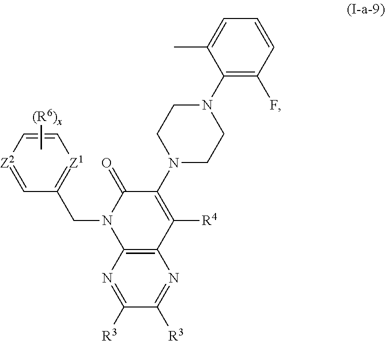

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, Z¹ and Z² are each independently CH, CR⁶, or N; each R⁶ is independently halogen, optionally substituted alkyl, or —OR^{A}; and x is 0, 1, 2, or 3.

In certain embodiments, the compound of Formula (I) is of Formula (I-a-10):

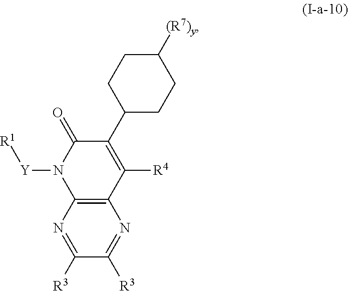

or a pharmaceutically acceptable salt, co-crystal, tautomer, stereoisomer, solvate, hydrate, polymorph, isotopically enriched derivative, or prodrug thereof; wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2; and Y, R¹, R³, and R⁴ are as defined herein.

In certain embodiments, each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; and y is 0, 1, or 2.

In certain embodiments, the compound of Formula (I) is of Formula (I-a-11):

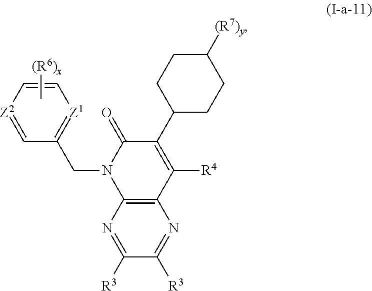

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; y is 0, 1, or 2; and R³ and R⁴ are as defined herein.

In certain embodiments, Z¹ and Z² are each independently CH, CR⁶, or N; each R⁶ is independently halogen, optionally substituted alkyl, or —OR^{A}; x is 0, 1, 2, or 3; each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; and y is 0, 1, or 2.

In certain embodiments, the compound of Formula (I) is of Formula (I-a-12):

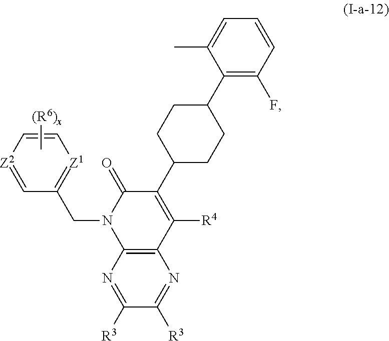

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, Z¹ and Z² are each independently CH, CR⁶, or N; each R⁶ is independently halogen, optionally substituted alkyl, or —OR^{A}; x is 0, 1, 2, or 3; and R³ and R⁴ are as defined herein.

In certain embodiments, the compound of Formula (I) is of Formula (I-b):

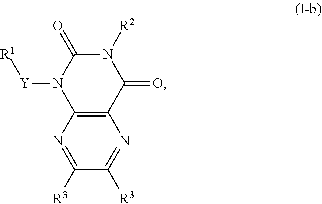

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-c):

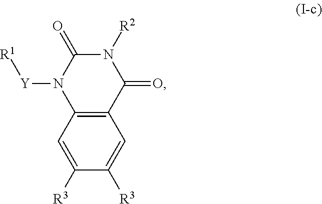

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-d):

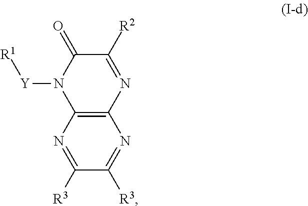

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-e):

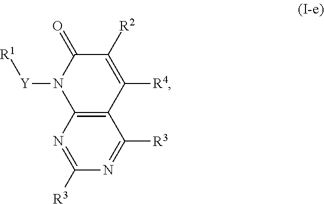

or a pharmaceutically acceptable salt, co-crystal, tautomer, stereoisomer, solvate, hydrate, polymorph, isotopically enriched derivative, or prodrug thereof; wherein R¹, Y, R², R³, and R⁴ are as defined herein.

In certain embodiments, the compound of Formula (I) is of Formula (I-e-1):

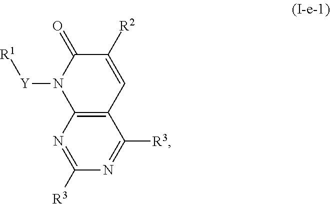

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-e-2):

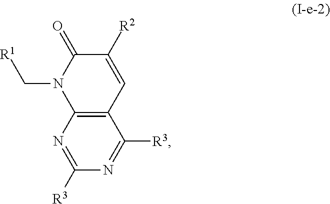

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-e-3):

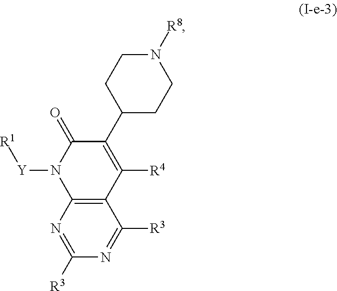

or a pharmaceutically acceptable salt, co-crystal, tautomer, stereoisomer, solvate, hydrate, polymorph, isotopically enriched derivative, or prodrug thereof; wherein R⁸ is optionally substituted alkyl, optionally substituted aryl, optionally substituted heteroaryl, or a nitrogen protecting group; and Y, R¹, R³, and R⁴ are as defined herein.

In certain embodiments, the compound of Formula (I) is of Formula (I-e-4):

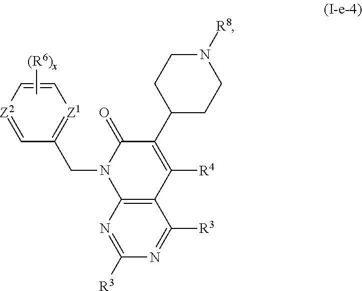

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; R⁸ is optionally substituted alkyl, optionally substituted aryl, optionally substituted heteroaryl, or a nitrogen protecting group; and R³ and R⁴ are as defined herein.

In certain embodiments, Z¹ and Z² are each independently CH, CR⁶, or N; each R⁶ is independently halogen, optionally substituted alkyl, or —OR^{A}; x is 0, 1, 2, or 3; and R⁸ is optionally substituted alkyl, optionally substituted aryl, optionally substituted heteroaryl, or a nitrogen protecting group.

In certain embodiments, the compound of Formula (I) is of Formula (I-e-5):

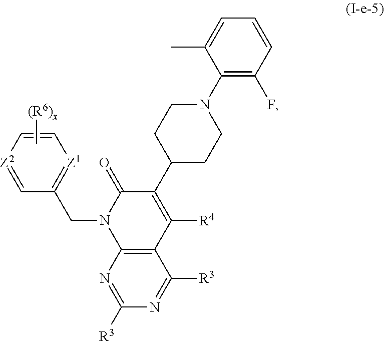

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, the compound of Formula (I) is of Formula (I-e-6):

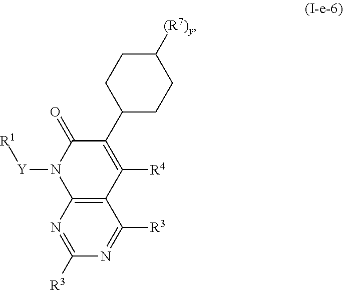

or a pharmaceutically acceptable salt, co-crystal, tautomer, stereoisomer, solvate, hydrate, polymorph, isotopically enriched derivative, or prodrug thereof; wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; y is 0, 1, or 2; and Y, R¹, R³, and R⁴ are as defined herein.

In certain embodiments, each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

In certain embodiments, the compound of Formula (I) is of Formula (I-e-7):

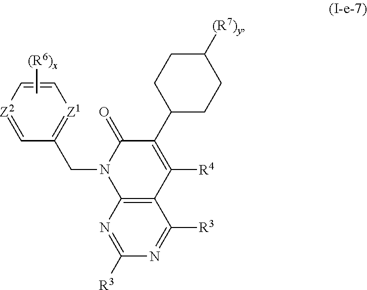

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; y is 0, 1, or 2; and R³ and R⁴ are as defined herein.

In certain embodiments, Z¹ and Z² are each independently CH, CR⁶, or N; each R⁶ is independently halogen, optionally substituted alkyl, or —OR^{A}; x is 0, 1, 2, or 3; each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; and y is 0, 1, or 2.

In certain embodiments, the compound of Formula (I) is of Formula (I-e-8):

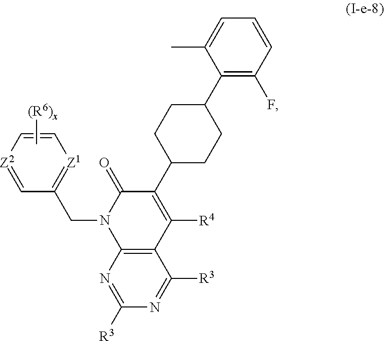

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; y is 0, 1, or 2; and R³ and R⁴ are as defined herein.

In certain embodiments, Z¹ and Z² are each independently CH, CR⁶, or N; each R⁶ is independently halogen, optionally substituted alkyl, optionally substituted cycloalkyl, or —OR^{A}; x is 0, 1, 2, or 3; each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; and y is 0, 1, or 2.

In certain embodiments, the compound of Formula (I) is of Formula (I-f):

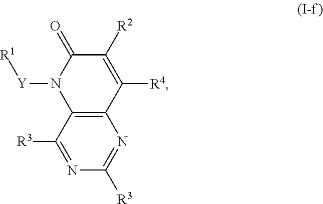

or a pharmaceutically acceptable salt, co-crystal, tautomer, stereoisomer, solvate, hydrate, polymorph, isotopically enriched derivative, or prodrug thereof; wherein R¹, Y, R², R³, and R⁴ are as defined herein.

In certain embodiments, the compound of Formula (I) is of Formula (I-f-1):

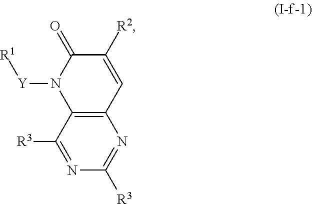

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-f-2):

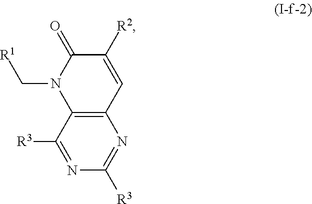

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-g):

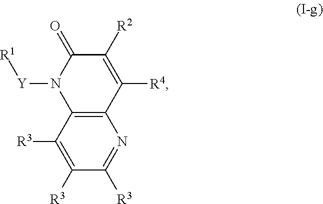

or a pharmaceutically acceptable salt, co-crystal, tautomer, stereoisomer, solvate, hydrate, polymorph, isotopically enriched derivative, or prodrug thereof; wherein R¹, Y, R², R³, and R⁴ are as defined herein.

In certain embodiments, the compound of Formula (I) is of Formula (I-g-1):

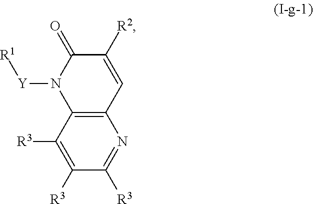

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-g-2):

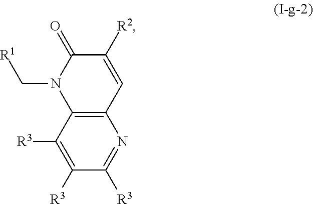

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-h):

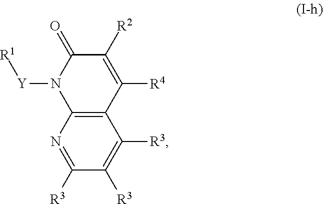

or a pharmaceutically acceptable salt, co-crystal, tautomer, stereoisomer, solvate, hydrate, polymorph, isotopically enriched derivative, or prodrug thereof; wherein R¹, Y, R², R³, and R⁴ are as defined herein.

In certain embodiments, the compound of Formula (I) is of Formula (I-h-1):

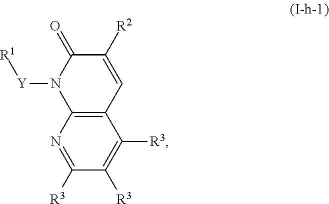

or a pharmaceutically acceptable salt, co-crystal, tautomer, stereoisomer, solvate, hydrate, polymorph, isotopically enriched derivative, or prodrug thereof; wherein R¹, Y, R², and R³ are as defined herein.

In certain embodiments, the compound of Formula (I) is of Formula (I-h-2):

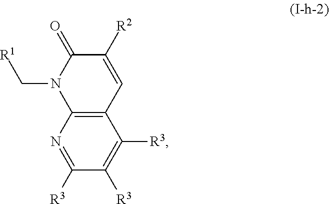

or a pharmaceutically acceptable salt, co-crystal, tautomer, stereoisomer, solvate, hydrate, polymorph, isotopically enriched derivative, or prodrug thereof; wherein R¹, R², and R³ are as defined herein.

In certain embodiments, the compound of Formula (I) is of Formula (I-a-3):

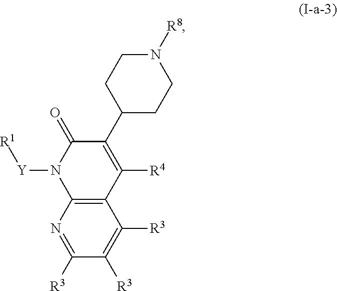

or a pharmaceutically acceptable salt, co-crystal, tautomer, stereoisomer, solvate, hydrate, polymorph, isotopically enriched derivative, or prodrug thereof; wherein R⁸ is optionally substituted alkyl, optionally substituted aryl, optionally substituted heteroaryl, or a nitrogen protecting group; and Y, R¹, R³, and R⁴ are as defined herein.

In certain embodiments, the compound of Formula (I) is of Formula (I-a-4):

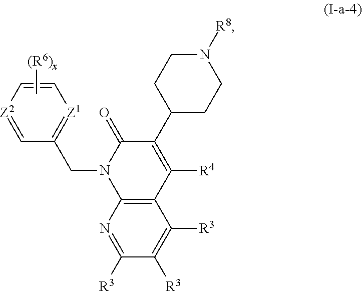

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; R⁸ is optionally substituted alkyl, optionally substituted aryl, optionally substituted heteroaryl, or a nitrogen protecting group; and R³ and R⁴ are as defined herein.

In certain embodiments, the compound of Formula (I) is of Formula (I-a-5):

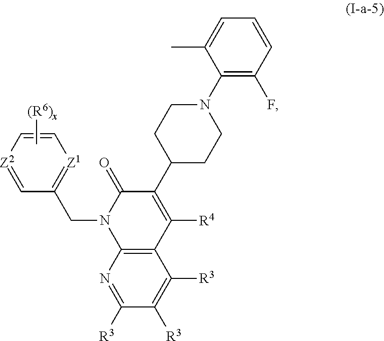

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, the compound of Formula (I) is of Formula (I-a-6):

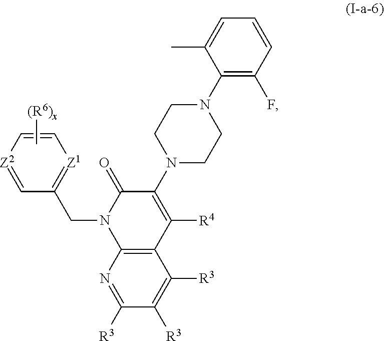

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, the compound of Formula (I) is of Formula (I-a-7):

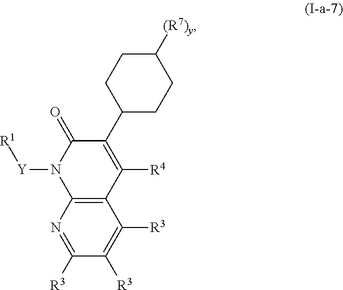

or a pharmaceutically acceptable salt, co-crystal, tautomer, stereoisomer, solvate, hydrate, polymorph, isotopically enriched derivative, or prodrug thereof; wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2; and Y, R¹, R³, and R⁴ are as defined herein.

In certain embodiments, the compound of Formula (I) is of Formula (I-a-8):

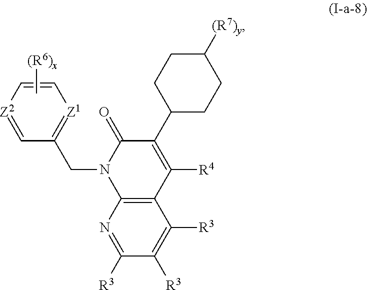

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; y is 0, 1, or 2; and R³ and R⁴ are as defined herein.

In certain embodiments, the compound of Formula (I) is of Formula (I-a-9):

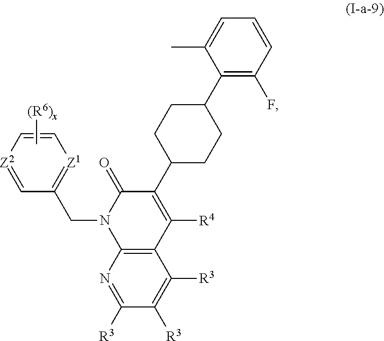

or a pharmaceutically acceptable salt, co-crystal, tautomer, stereoisomer, solvate, hydrate, polymorph, isotopically enriched derivative, or prodrug thereof; wherein Z¹ and Z² are each independently CH, CR⁶, or N; each R⁶ is independently halogen, optionally substituted alkyl, optionally substituted cycloalkyl, or —OR^{A}; x is 0, 1, 2, or 3; and R³ and R⁴ are as defined herein.

In certain embodiments, the compound of Formula (I) is one of the following compounds, or a pharmaceutically acceptable salt, co-crystal, tautomer, stereoisomer, solvate, hydrate, polymorph, isotopically enriched derivative, or prodrug thereof:

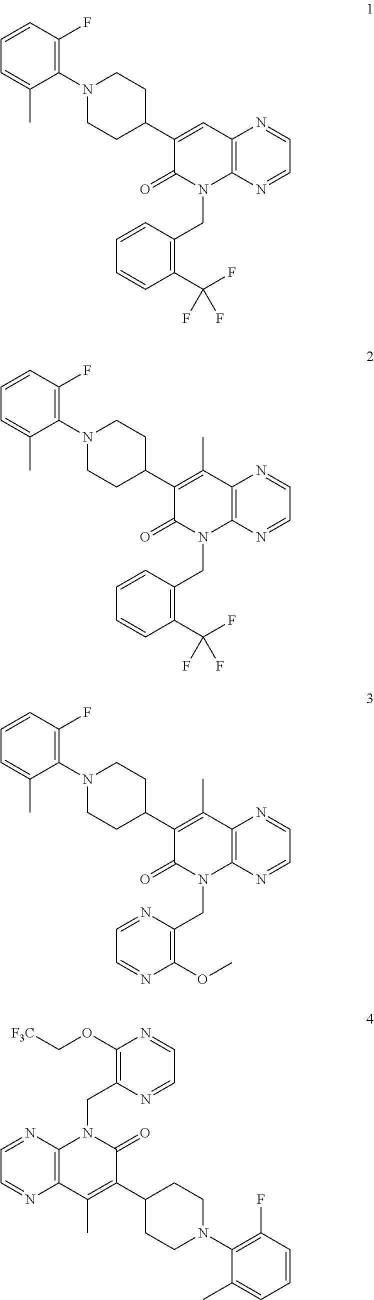 [SMILES: Cc1cccc(F)c1N1CCC(c2cc3nccnc3n(Cc3ccccc3C(F)(F)F)c2=O)CC1 ; Cc1cccc(F)c1N1CCC(c2c(C)c3nccnc3n(Cc3ccccc3C(F)(F)F)c2=O)CC1 ; COc1nccnc1Cn1c(=O)c(C2CCN(c3c(C)cccc3F)CC2)c(C)c2nccnc21 ; Cc1cccc(F)c1N1CCC(c2c(C)c3nccnc3n(Cc3nccnc3OCC(F)(F)F)c2=O)CC1]

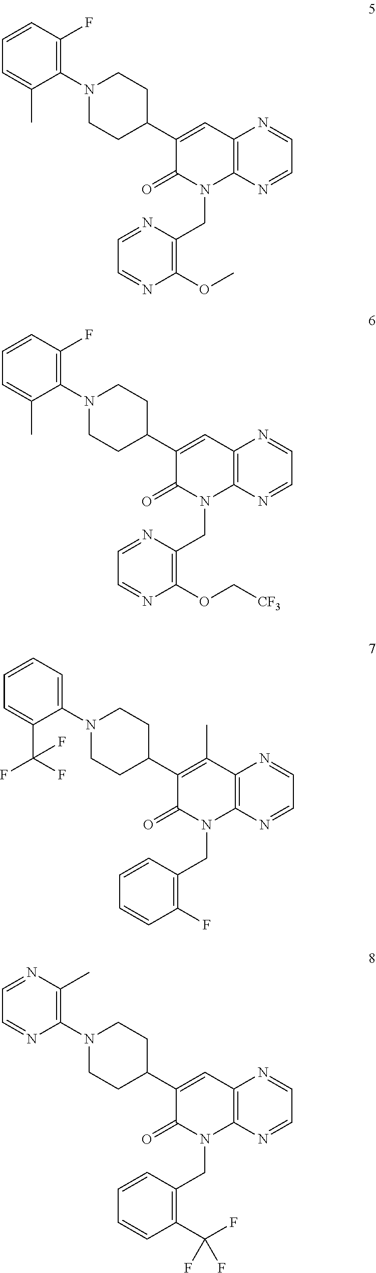 [SMILES: COc1nccnc1Cn1c(=O)c(C2CCN(c3c(C)cccc3F)CC2)cc2nccnc21 ; Cc1cccc(F)c1N1CCC(c2cc3nccnc3n(Cc3nccnc3OCC(F)(F)F)c2=O)CC1 ; Cc1c(C2CCN(c3ccccc3C(F)(F)F)CC2)c(=O)n(Cc2ccccc2F)c2nccnc12 ; Cc1nccnc1N1CCC(c2cc3nccnc3n(Cc3ccccc3C(F)(F)F)c2=O)CC1]

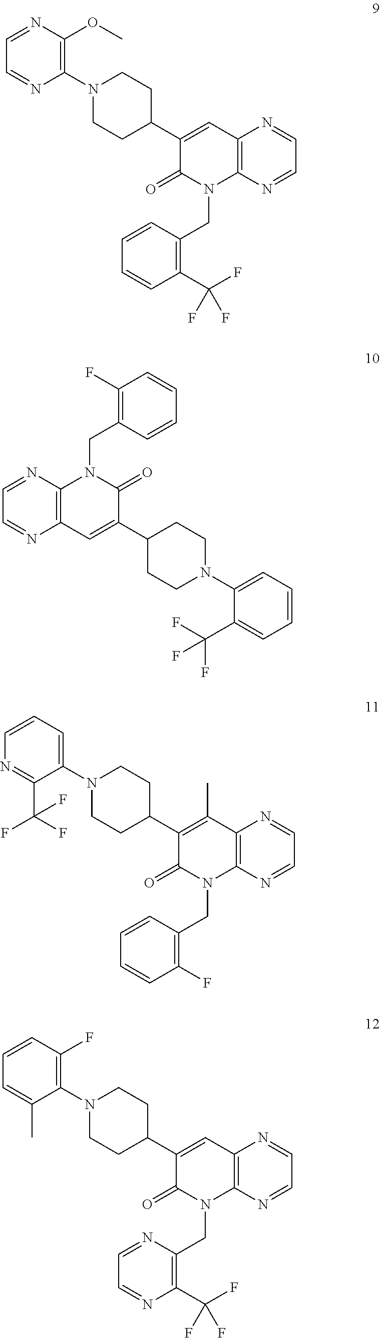 [SMILES: COc1nccnc1N1CCC(c2cc3nccnc3n(Cc3ccccc3C(F)(F)F)c2=O)CC1 ; O=c1c(C2CCN(c3ccccc3C(F)(F)F)CC2)cc2nccnc2n1Cc1ccccc1F ; Cc1c(C2CCN(c3cccnc3C(F)(F)F)CC2)c(=O)n(Cc2ccccc2F)c2nccnc12 ; Cc1cccc(F)c1N1CCC(c2cc3nccnc3n(Cc3nccnc3C(F)(F)F)c2=O)CC1]

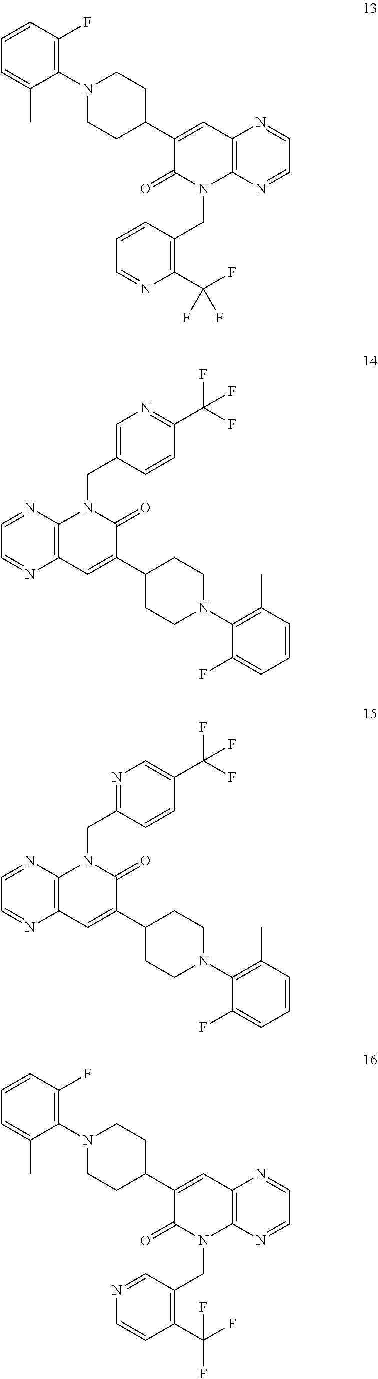 [SMILES: Cc1cccc(F)c1N1CCC(c2cc3nccnc3n(Cc3cccnc3C(F)(F)F)c2=O)CC1 ; Cc1cccc(F)c1N1CCC(c2cc3nccnc3n(Cc3ccc(C(F)(F)F)nc3)c2=O)CC1 ; Cc1cccc(F)c1N1CCC(c2cc3nccnc3n(Cc3ccc(C(F)(F)F)cn3)c2=O)CC1 ; Cc1cccc(F)c1N1CCC(c2cc3nccnc3n(Cc3cnccc3C(F)(F)F)c2=O)CC1]

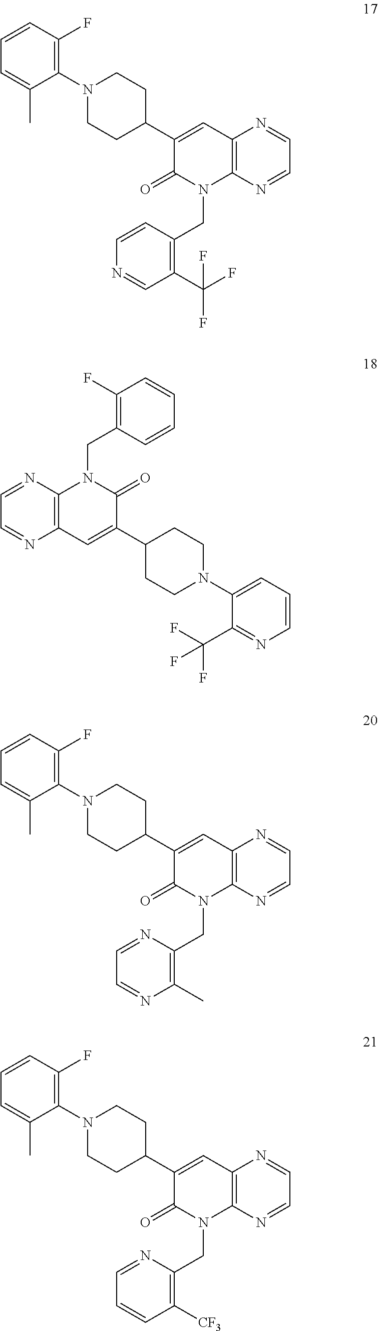 [SMILES: Cc1cccc(F)c1N1CCC(c2cc3nccnc3n(Cc3ccncc3C(F)(F)F)c2=O)CC1 ; O=c1c(C2CCN(c3cccnc3C(F)(F)F)CC2)cc2nccnc2n1Cc1ccccc1F ; Cc1cccc(F)c1N1CCC(c2cc3nccnc3n(Cc3nccnc3C)c2=O)CC1 ; Cc1cccc(F)c1N1CCC(c2cc3nccnc3n(Cc3ncccc3C(F)(F)F)c2=O)CC1]

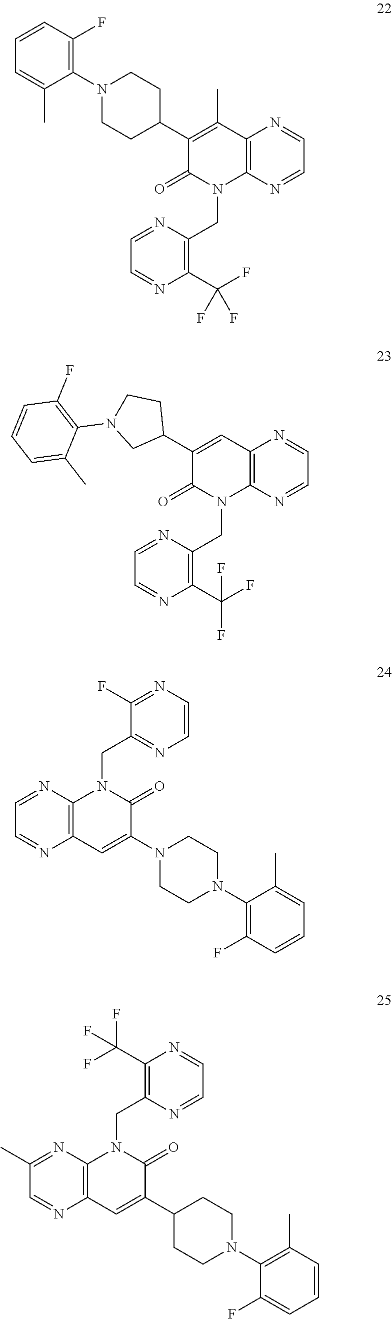 [SMILES: Cc1cccc(F)c1N1CCC(c2c(C)c3nccnc3n(Cc3nccnc3C(F)(F)F)c2=O)CC1 ; Cc1cccc(F)c1N1CCC(c2cc3nccnc3n(Cc3nccnc3C(F)(F)F)c2=O)C1 ; Cc1cccc(F)c1N1CCN(c2cc3nccnc3n(Cc3nccnc3F)c2=O)CC1 ; Cc1cnc2cc(C3CCN(c4c(C)cccc4F)CC3)c(=O)n(Cc3nccnc3C(F)(F)F)c2n1]

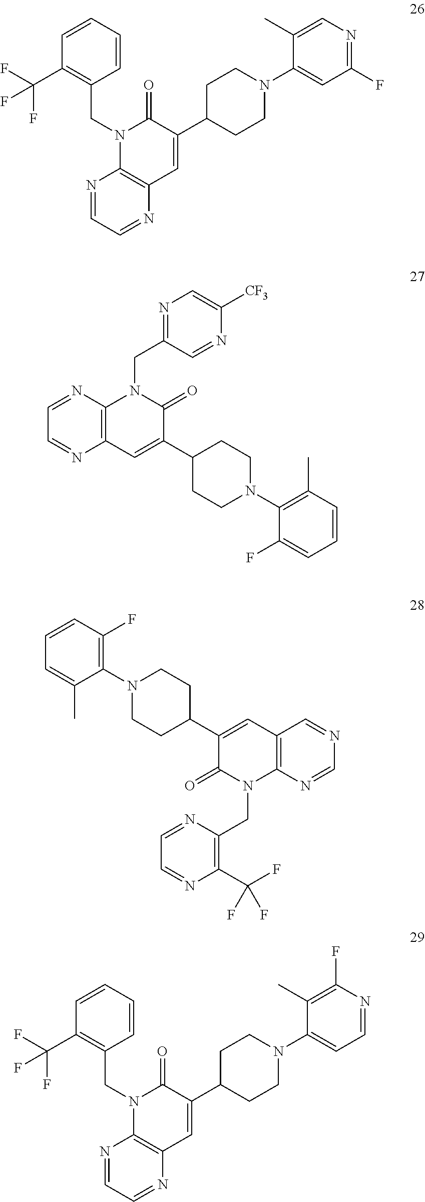 [SMILES: Cc1cnc(F)cc1N1CCC(c2cc3nccnc3n(Cc3ccccc3C(F)(F)F)c2=O)CC1 ; Cc1cccc(F)c1N1CCC(c2cc3nccnc3n(Cc3cnc(C(F)(F)F)cn3)c2=O)CC1 ; Cc1cccc(F)c1N1CCC(c2cc3cncnc3n(Cc3nccnc3C(F)(F)F)c2=O)CC1 ; Cc1c(N2CCC(c3cc4nccnc4n(Cc4ccccc4C(F)(F)F)c3=O)CC2)ccnc1F]

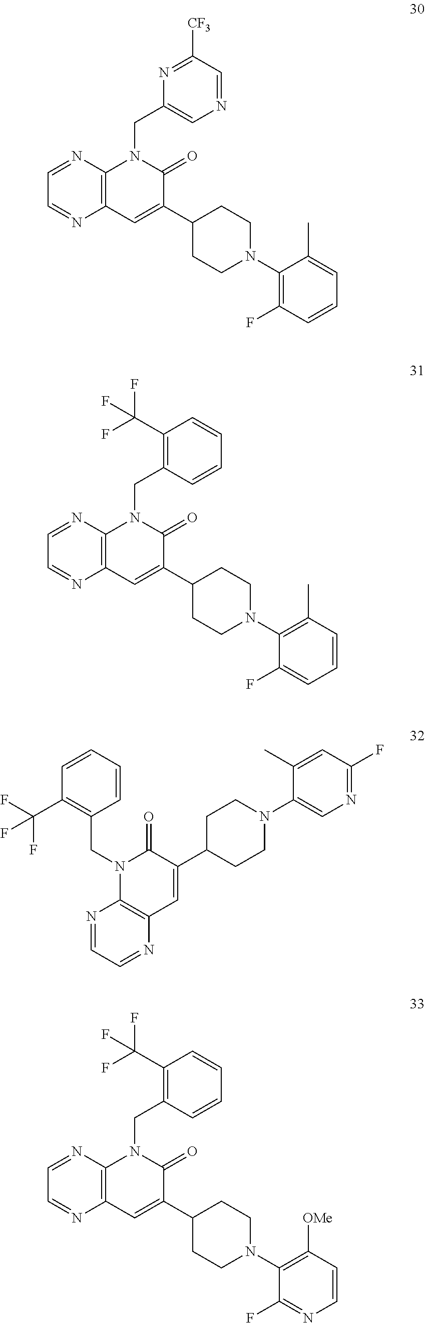 [SMILES: Cc1cccc(F)c1N1CCC(c2cc3nccnc3n(Cc3cncc(C(F)(F)F)n3)c2=O)CC1 ; Cc1cccc(F)c1N1CCC(c2cc3nccnc3n(Cc3ccccc3C(F)(F)F)c2=O)CC1 ; Cc1cc(F)ncc1N1CCC(c2cc3nccnc3n(Cc3ccccc3C(F)(F)F)c2=O)CC1 ; COc1ccnc(F)c1N1CCC(c2cc3nccnc3n(Cc3ccccc3C(F)(F)F)c2=O)CC1]

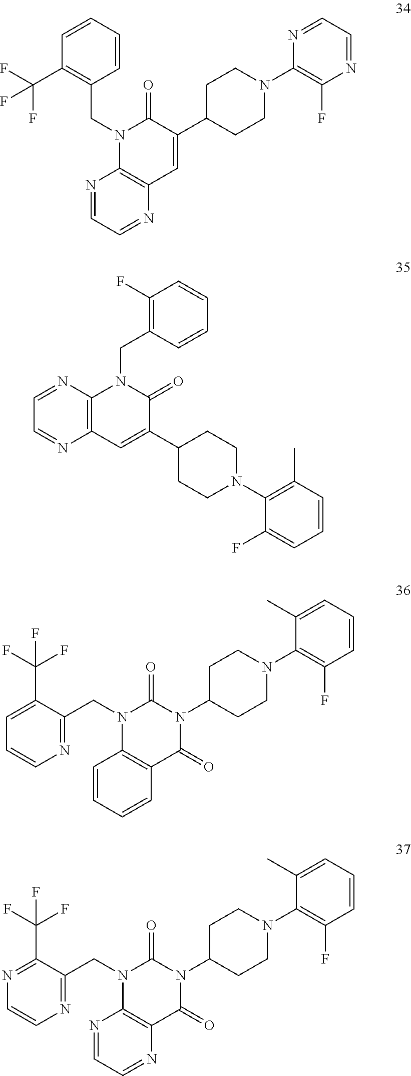

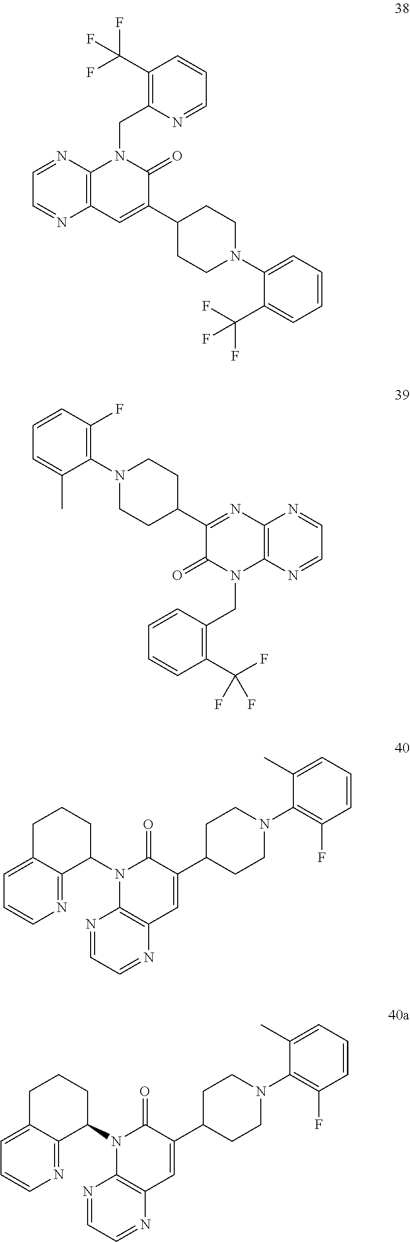 [SMILES: O=c1c(C2CCN(c3ccccc3C(F)(F)F)CC2)cc2nccnc2n1Cc1ncccc1C(F)(F)F ; Cc1cccc(F)c1N1CCC(c2nc3nccnc3n(Cc3ccccc3C(F)(F)F)c2=O)CC1 ; Cc1cccc(F)c1N1CCC(c2cc3nccnc3n(C3CCCc4cccnc43)c2=O)CC1 ; Cc1cccc(F)c1N1CCC(c2cc3nccnc3n([C@@H]3CCCc4cccnc43)c2=O)CC1]

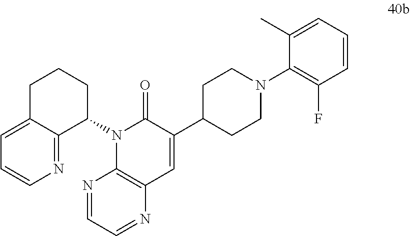 [SMILES: Cc1cccc(F)c1N1CCC(c2cc3nccnc3n([C@H]3CCCc4cccnc43)c2=O)CC1]

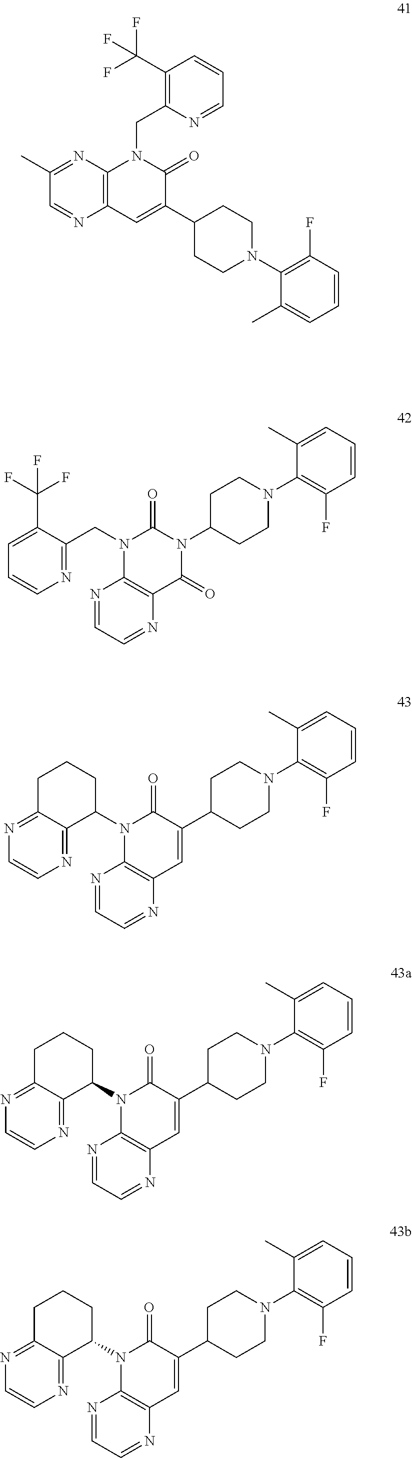 [SMILES: Cc1cnc2cc(C3CCN(c4c(C)cccc4F)CC3)c(=O)n(Cc3ncccc3C(F)(F)F)c2n1 ; Cc1cccc(F)c1N1CCC(n2c(=O)c3nccnc3n(Cc3ncccc3C(F)(F)F)c2=O)CC1 ; Cc1cccc(F)c1N1CCC(c2cc3nccnc3n(C3CCCc4nccnc43)c2=O)CC1 ; Cc1cccc(F)c1N1CCC(c2cc3nccnc3n([C@@H]3CCCc4nccnc43)c2=O)CC1 ; Cc1cccc(F)c1N1CCC(c2cc3nccnc3n([C@H]3CCCc4nccnc43)c2=O)CC1]

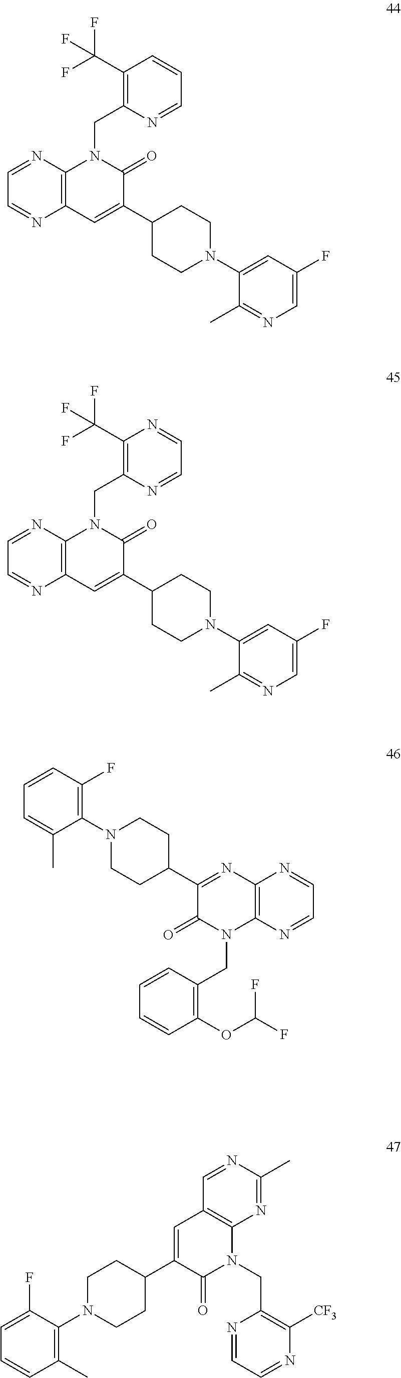 [SMILES: Cc1ncc(F)cc1N1CCC(c2cc3nccnc3n(Cc3ncccc3C(F)(F)F)c2=O)CC1 ; Cc1ncc(F)cc1N1CCC(c2cc3nccnc3n(Cc3nccnc3C(F)(F)F)c2=O)CC1 ; Cc1cccc(F)c1N1CCC(c2nc3nccnc3n(Cc3ccccc3OC(F)F)c2=O)CC1 ; Cc1ncc2cc(C3CCN(c4c(C)cccc4F)CC3)c(=O)n(Cc3nccnc3C(F)(F)F)c2n1]

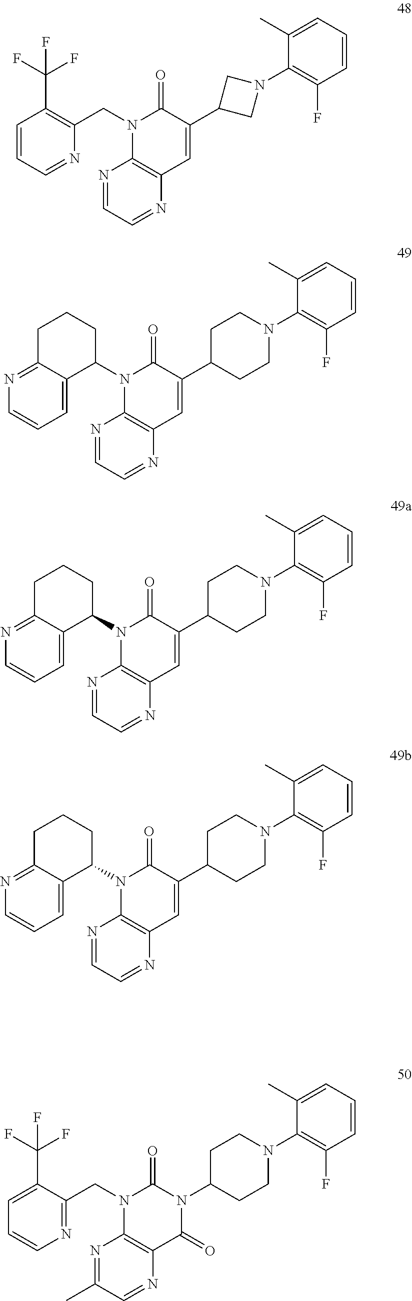 [SMILES: Cc1cccc(F)c1N1CC(c2cc3nccnc3n(Cc3ncccc3C(F)(F)F)c2=O)C1 ; Cc1cccc(F)c1N1CCC(c2cc3nccnc3n(C3CCCc4ncccc43)c2=O)CC1 ; Cc1cccc(F)c1N1CCC(c2cc3nccnc3n([C@@H]3CCCc4ncccc43)c2=O)CC1 ; Cc1cccc(F)c1N1CCC(c2cc3nccnc3n([C@H]3CCCc4ncccc43)c2=O)CC1 ; Cc1cnc2c(=O)n(C3CCN(c4c(C)cccc4F)CC3)c(=O)n(Cc3ncccc3C(F)(F)F)c2n1]

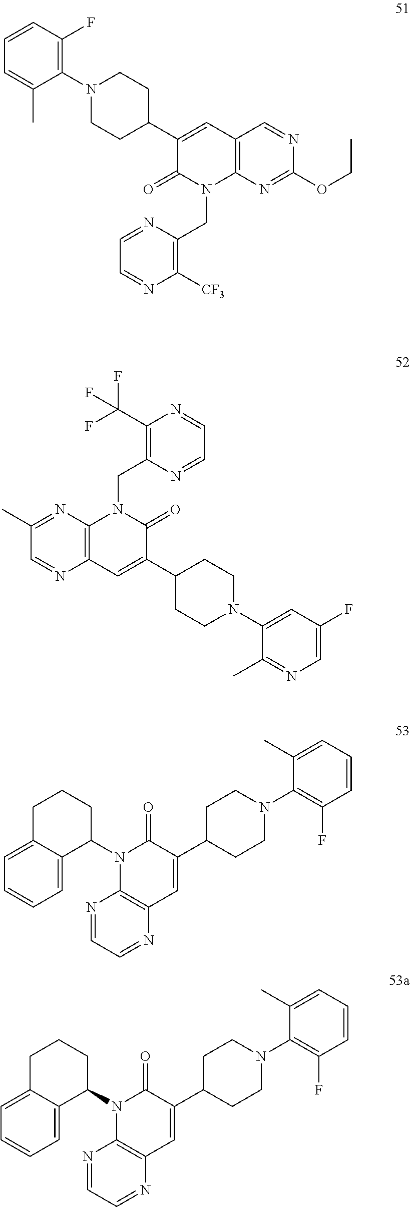

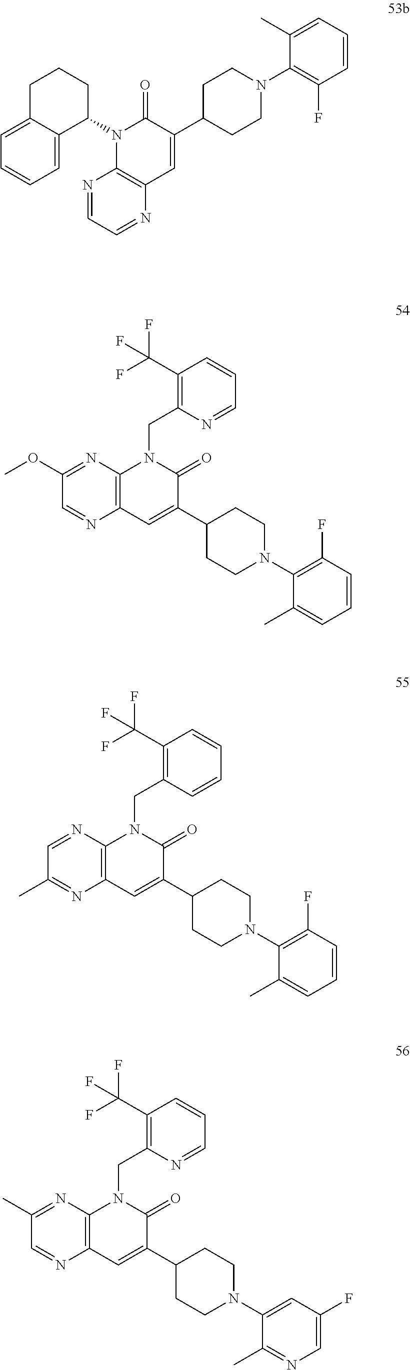 [SMILES: Cc1cccc(F)c1N1CCC(c2cc3nccnc3n([C@H]3CCCc4ccccc43)c2=O)CC1 ; COc1cnc2cc(C3CCN(c4c(C)cccc4F)CC3)c(=O)n(Cc3ncccc3C(F)(F)F)c2n1 ; Cc1cnc2c(cc(C3CCN(c4c(C)cccc4F)CC3)c(=O)n2Cc2ccccc2C(F)(F)F)n1 ; Cc1cnc2cc(C3CCN(c4cc(F)cnc4C)CC3)c(=O)n(Cc3ncccc3C(F)(F)F)c2n1]

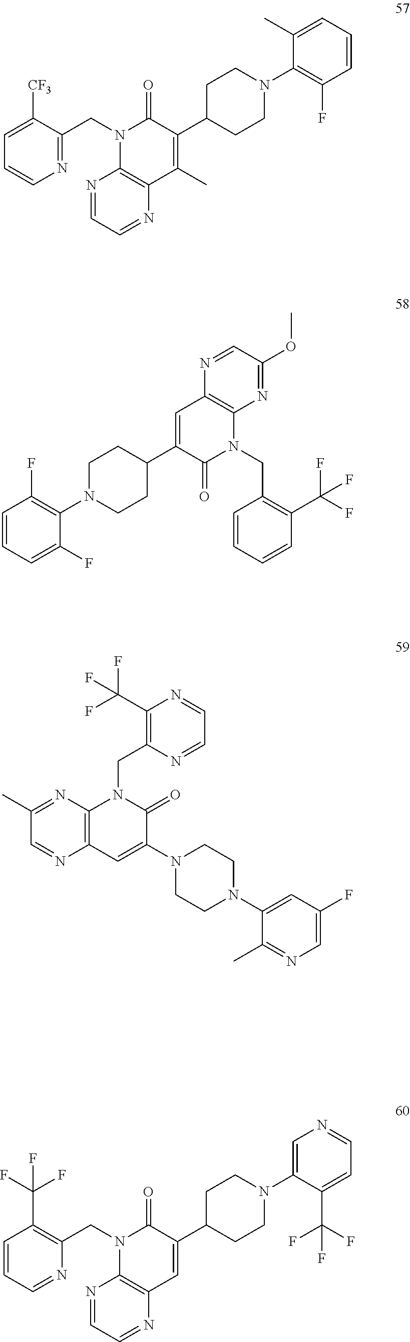 [SMILES: Cc1cccc(F)c1N1CCC(c2c(C)c3nccnc3n(Cc3ncccc3C(F)(F)F)c2=O)CC1 ; COc1cnc2cc(C3CCN(c4c(F)cccc4F)CC3)c(=O)n(Cc3ccccc3C(F)(F)F)c2n1 ; Cc1cnc2cc(N3CCN(c4cc(F)cnc4C)CC3)c(=O)n(Cc3nccnc3C(F)(F)F)c2n1 ; O=c1c(C2CCN(c3cnccc3C(F)(F)F)CC2)cc2nccnc2n1Cc1ncccc1C(F)(F)F]

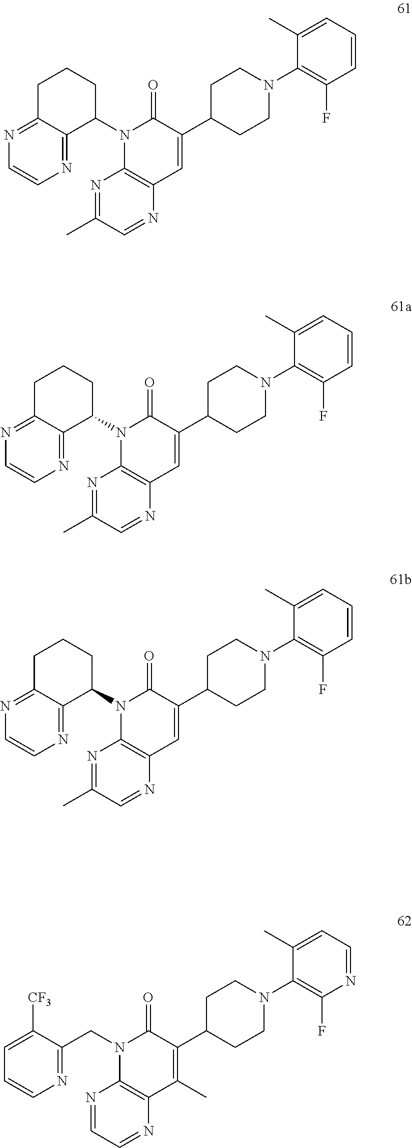 [SMILES: Cc1cnc2cc(C3CCN(c4c(C)cccc4F)CC3)c(=O)n(C3CCCc4nccnc43)c2n1 ; Cc1cnc2cc(C3CCN(c4c(C)cccc4F)CC3)c(=O)n([C@H]3CCCc4nccnc43)c2n1 ; Cc1cnc2cc(C3CCN(c4c(C)cccc4F)CC3)c(=O)n([C@@H]3CCCc4nccnc43)c2n1 ; Cc1ccnc(F)c1N1CCC(c2c(C)c3nccnc3n(Cc3ncccc3C(F)(F)F)c2=O)CC1]

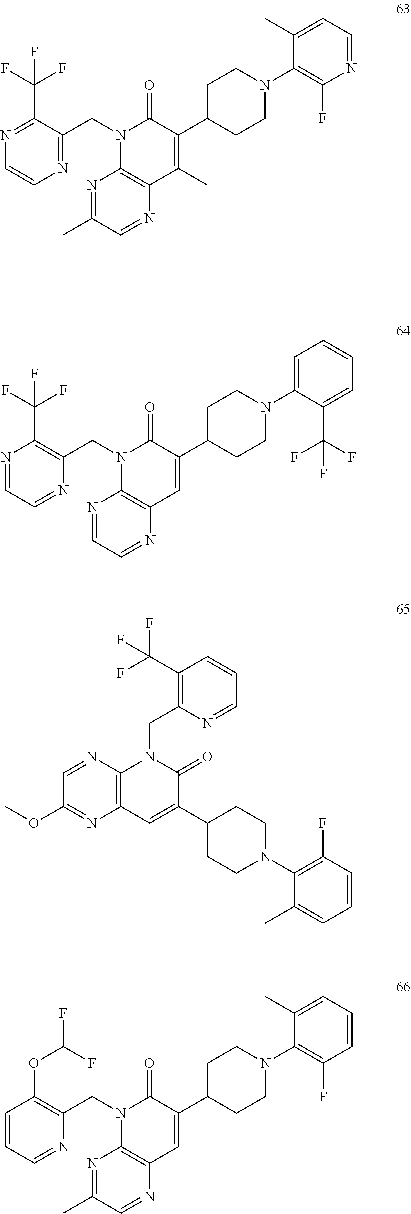 [SMILES: Cc1cnc2c(C)c(C3CCN(c4c(C)ccnc4F)CC3)c(=O)n(Cc3nccnc3C(F)(F)F)c2n1 ; O=c1c(C2CCN(c3ccccc3C(F)(F)F)CC2)cc2nccnc2n1Cc1nccnc1C(F)(F)F ; COc1cnc2c(cc(C3CCN(c4c(C)cccc4F)CC3)c(=O)n2Cc2ncccc2C(F)(F)F)n1 ; Cc1cnc2cc(C3CCN(c4c(C)cccc4F)CC3)c(=O)n(Cc3ncccc3OC(F)F)c2n1]

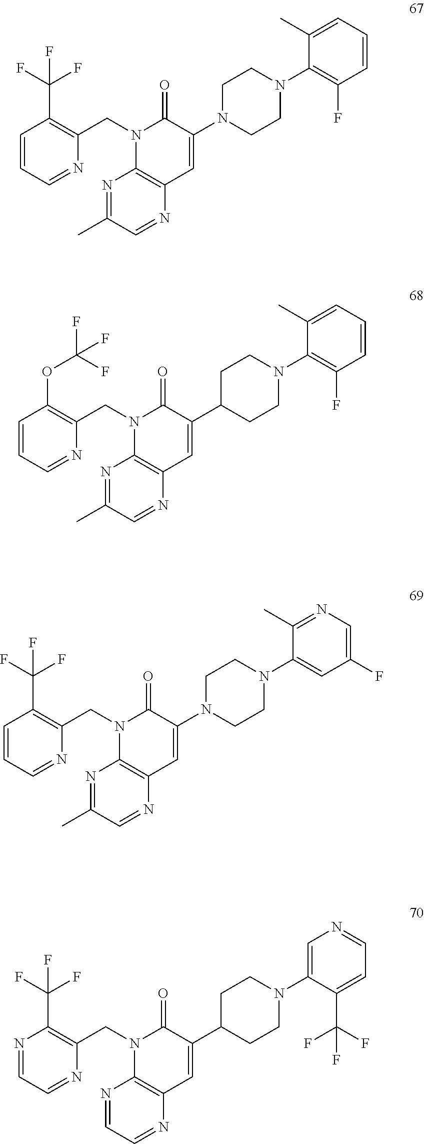 [SMILES: Cc1cnc2cc(N3CCN(c4c(C)cccc4F)CC3)c(=O)n(Cc3ncccc3C(F)(F)F)c2n1 ; Cc1cnc2cc(C3CCN(c4c(C)cccc4F)CC3)c(=O)n(Cc3ncccc3OC(F)(F)F)c2n1 ; Cc1cnc2cc(N3CCN(c4cc(F)cnc4C)CC3)c(=O)n(Cc3ncccc3C(F)(F)F)c2n1 ; O=c1c(C2CCN(c3cnccc3C(F)(F)F)CC2)cc2nccnc2n1Cc1nccnc1C(F)(F)F]

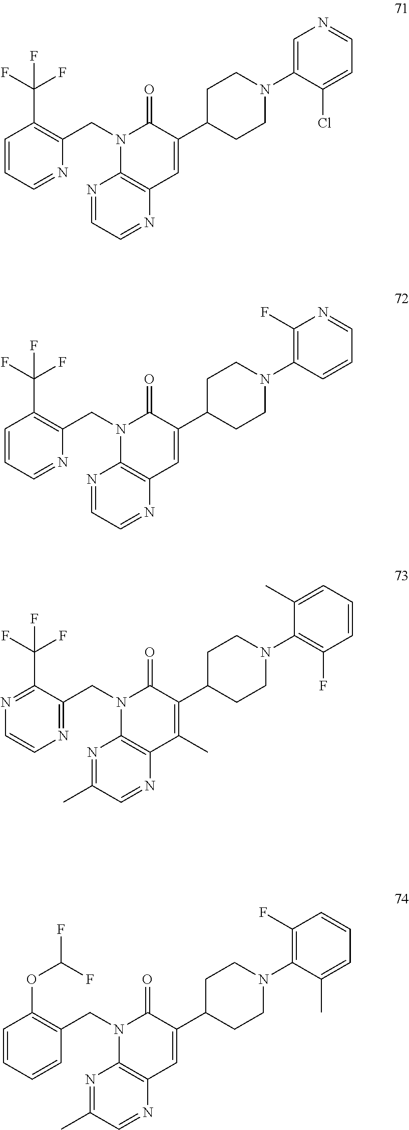 [SMILES: O=c1c(C2CCN(c3cnccc3Cl)CC2)cc2nccnc2n1Cc1ncccc1C(F)(F)F ; O=c1c(C2CCN(c3cccnc3F)CC2)cc2nccnc2n1Cc1ncccc1C(F)(F)F ; Cc1cnc2c(C)c(C3CCN(c4c(C)cccc4F)CC3)c(=O)n(Cc3nccnc3C(F)(F)F)c2n1 ; Cc1cnc2cc(C3CCN(c4c(C)cccc4F)CC3)c(=O)n(Cc3ccccc3OC(F)F)c2n1]

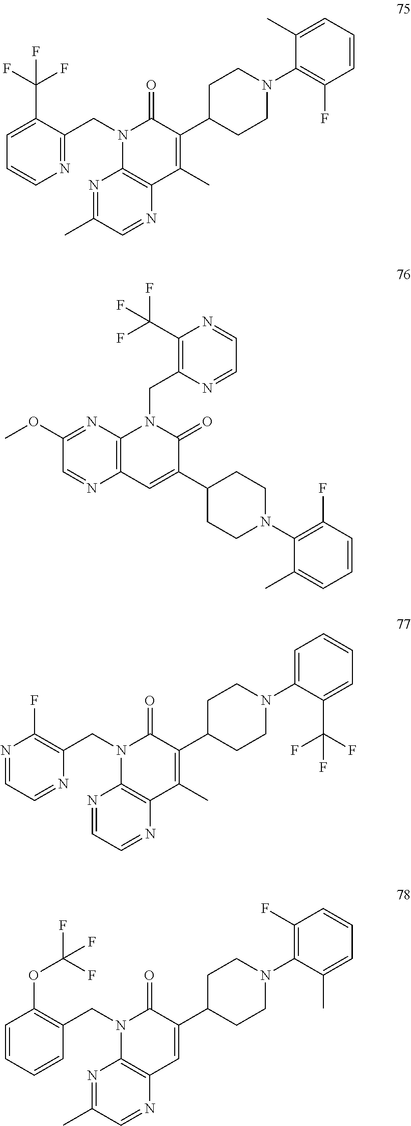

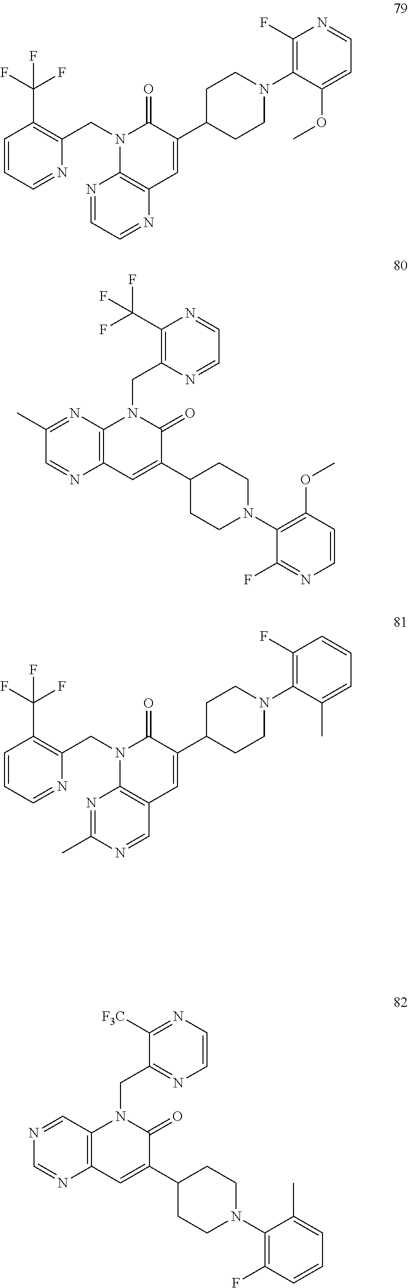

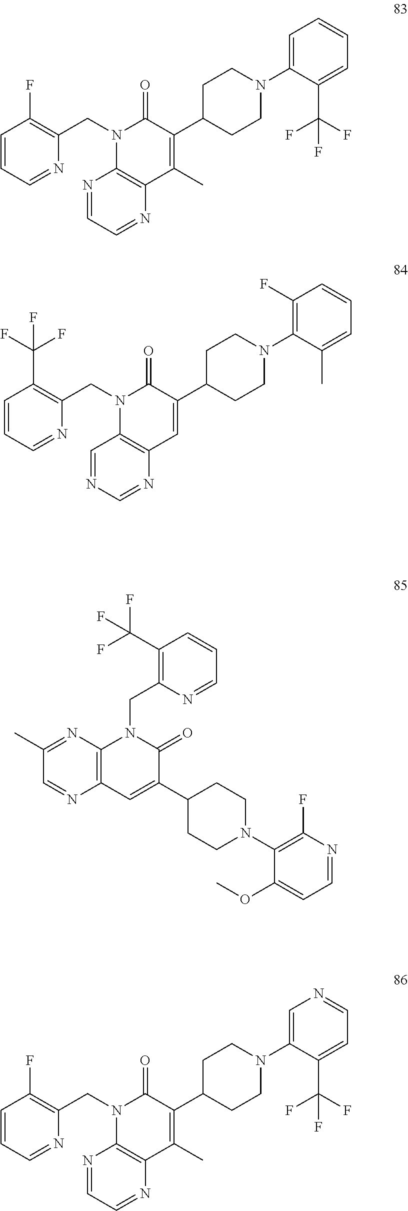 [SMILES: Cc1c(C2CCN(c3ccccc3C(F)(F)F)CC2)c(=O)n(Cc2ncccc2F)c2nccnc12 ; Cc1cccc(F)c1N1CCC(c2cc3ncncc3n(Cc3ncccc3C(F)(F)F)c2=O)CC1 ; COc1ccnc(F)c1N1CCC(c2cc3ncc(C)nc3n(Cc3ncccc3C(F)(F)F)c2=O)CC1 ; Cc1c(C2CCN(c3cnccc3C(F)(F)F)CC2)c(=O)n(Cc2ncccc2F)c2nccnc12]

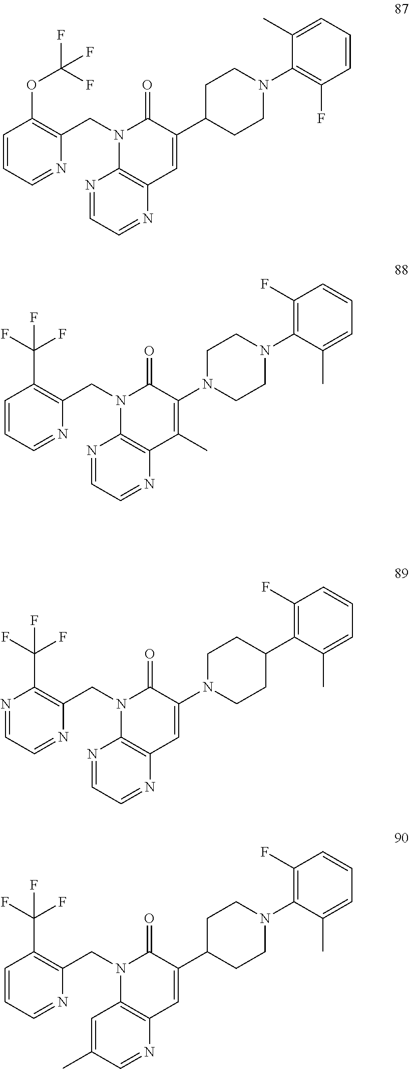 [SMILES: Cc1cccc(F)c1N1CCC(c2cc3nccnc3n(Cc3ncccc3OC(F)(F)F)c2=O)CC1 ; Cc1cccc(F)c1N1CCN(c2c(C)c3nccnc3n(Cc3ncccc3C(F)(F)F)c2=O)CC1 ; Cc1cccc(F)c1C1CCN(c2cc3nccnc3n(Cc3nccnc3C(F)(F)F)c2=O)CC1 ; Cc1cnc2cc(C3CCN(c4c(C)cccc4F)CC3)c(=O)n(Cc3ncccc3C(F)(F)F)c2c1]

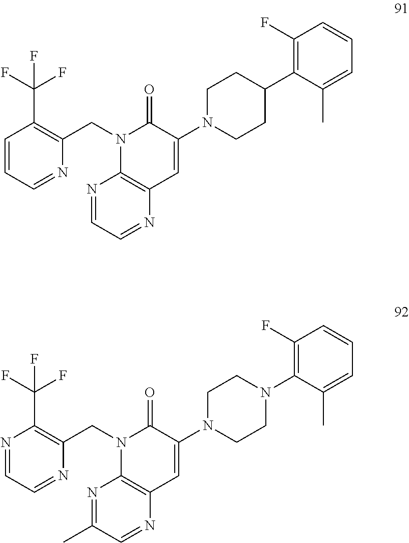 [SMILES: Cc1cccc(F)c1C1CCN(c2cc3nccnc3n(Cc3ncccc3C(F)(F)F)c2=O)CC1 ; Cc1cnc2cc(N3CCN(c4c(C)cccc4F)CC3)c(=O)n(Cc3nccnc3C(F)(F)F)c2n1]

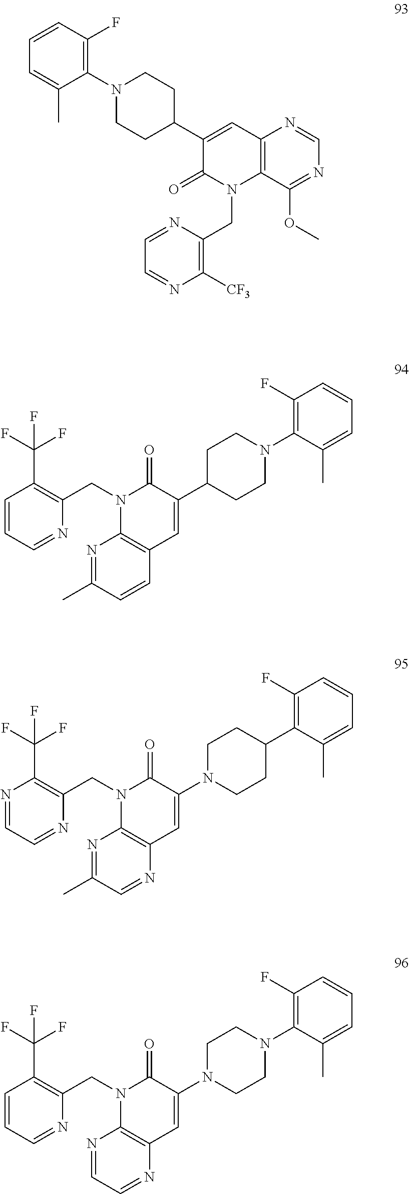 [SMILES: COc1ncnc2cc(C3CCN(c4c(C)cccc4F)CC3)c(=O)n(Cc3nccnc3C(F)(F)F)c12 ; Cc1ccc2cc(C3CCN(c4c(C)cccc4F)CC3)c(=O)n(Cc3ncccc3C(F)(F)F)c2n1 ; Cc1cnc2cc(N3CCC(c4c(C)cccc4F)CC3)c(=O)n(Cc3nccnc3C(F)(F)F)c2n1 ; Cc1cccc(F)c1N1CCN(c2cc3nccnc3n(Cc3ncccc3C(F)(F)F)c2=O)CC1]

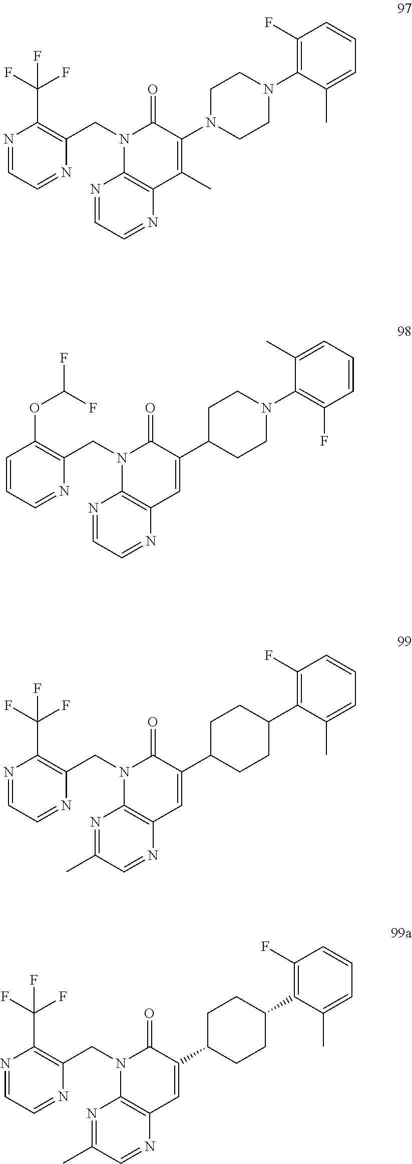 [SMILES: Cc1cccc(F)c1N1CCN(c2c(C)c3nccnc3n(Cc3nccnc3C(F)(F)F)c2=O)CC1 ; Cc1cccc(F)c1N1CCC(c2cc3nccnc3n(Cc3ncccc3OC(F)F)c2=O)CC1 ; Cc1cnc2cc(C3CCC(c4c(C)cccc4F)CC3)c(=O)n(Cc3nccnc3C(F)(F)F)c2n1 ; Cc1cnc2cc([C@H]3CC[C@@H](c4c(C)cccc4F)CC3)c(=O)n(Cc3nccnc3C(F)(F)F)c2n1]

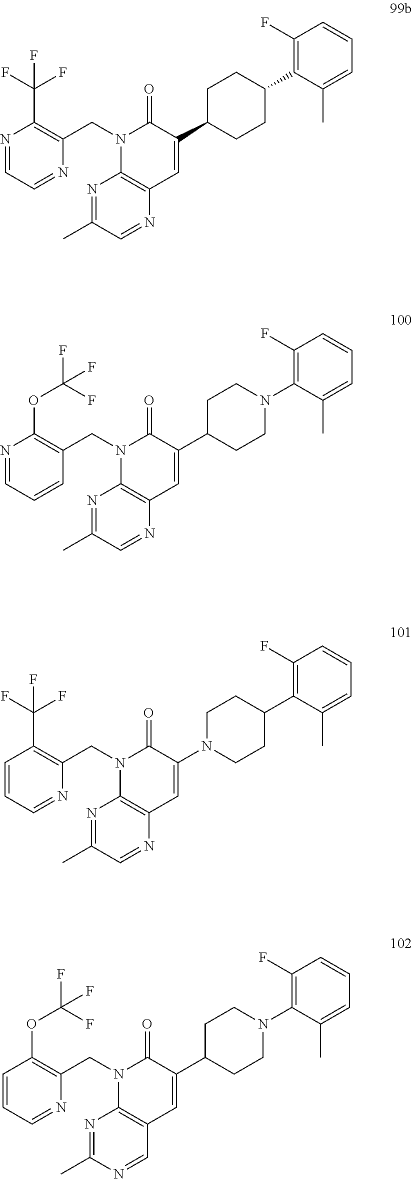 [SMILES: Cc1cnc2cc([C@H]3CC[C@H](c4c(C)cccc4F)CC3)c(=O)n(Cc3nccnc3C(F)(F)F)c2n1 ; Cc1cnc2cc(C3CCN(c4c(C)cccc4F)CC3)c(=O)n(Cc3cccnc3OC(F)(F)F)c2n1 ; Cc1cnc2cc(N3CCC(c4c(C)cccc4F)CC3)c(=O)n(Cc3ncccc3C(F)(F)F)c2n1 ; Cc1ncc2cc(C3CCN(c4c(C)cccc4F)CC3)c(=O)n(Cc3ncccc3OC(F)(F)F)c2n1]

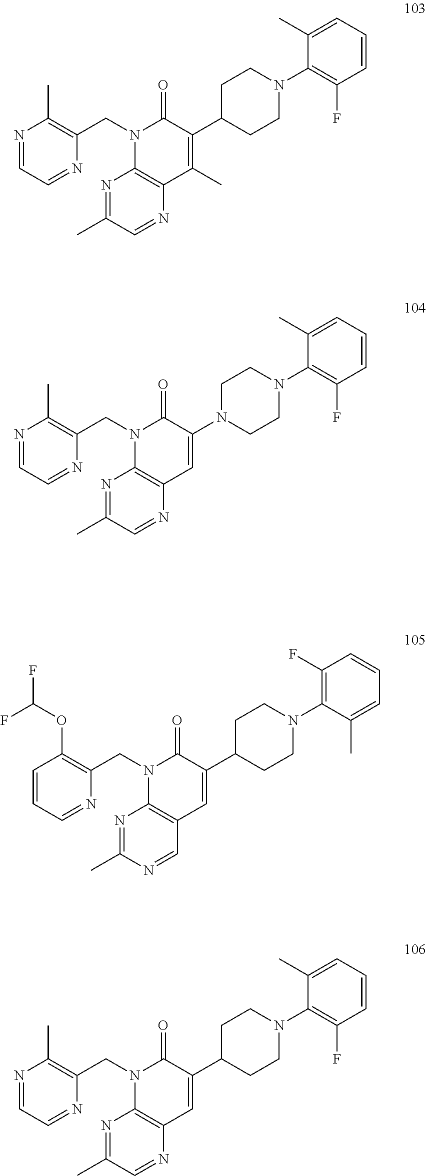 [SMILES: Cc1cnc2c(C)c(C3CCN(c4c(C)cccc4F)CC3)c(=O)n(Cc3nccnc3C)c2n1 ; Cc1cnc2cc(N3CCN(c4c(C)cccc4F)CC3)c(=O)n(Cc3nccnc3C)c2n1 ; Cc1ncc2cc(C3CCN(c4c(C)cccc4F)CC3)c(=O)n(Cc3ncccc3OC(F)F)c2n1 ; Cc1cnc2cc(C3CCN(c4c(C)cccc4F)CC3)c(=O)n(Cc3nccnc3C)c2n1]

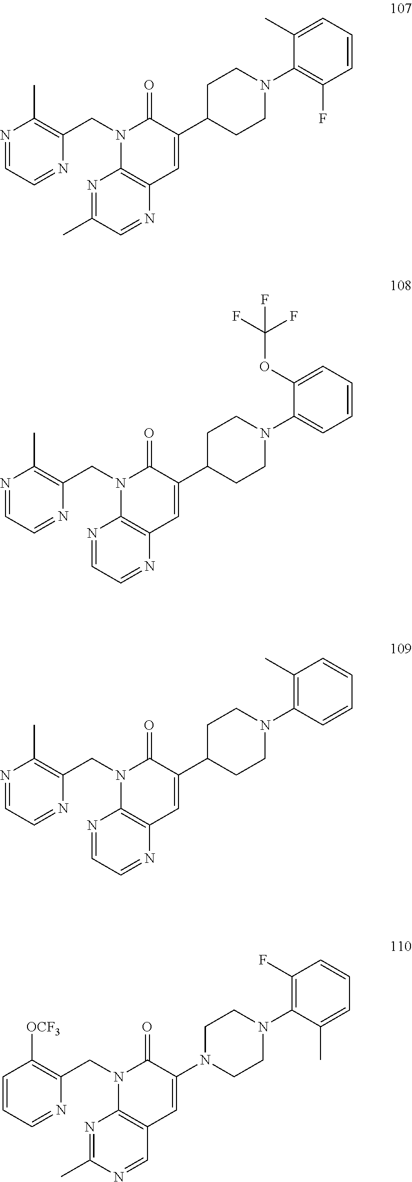 [SMILES: Cc1cnc2cc(C3CCN(c4c(C)cccc4F)CC3)c(=O)n(Cc3nccnc3C)c2n1 ; Cc1nccnc1Cn1c(=O)c(C2CCN(c3ccccc3OC(F)(F)F)CC2)cc2nccnc21 ; Cc1ccccc1N1CCC(c2cc3nccnc3n(Cc3nccnc3C)c2=O)CC1 ; Cc1ncc2cc(N3CCN(c4c(C)cccc4F)CC3)c(=O)n(Cc3ncccc3OC(F)(F)F)c2n1]

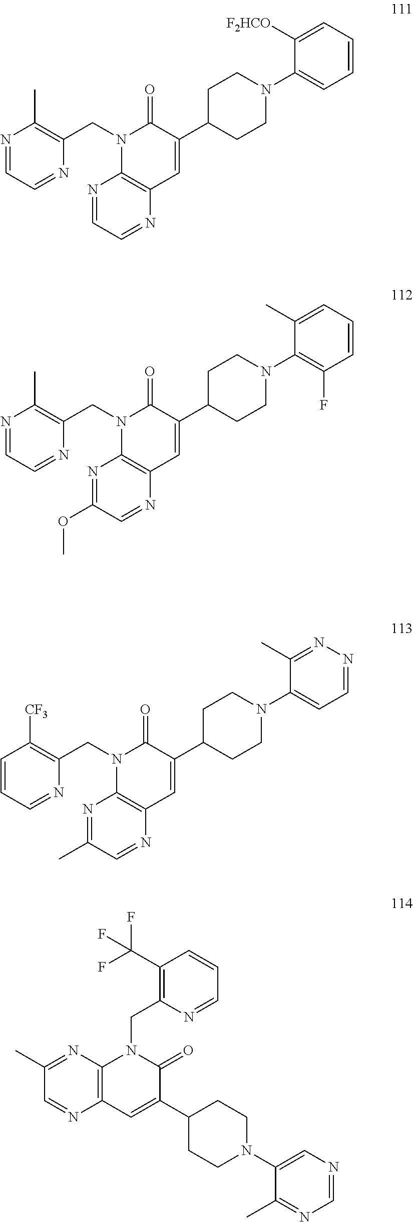 [SMILES: Cc1nccnc1Cn1c(=O)c(C2CCN(c3ccccc3OC(F)F)CC2)cc2nccnc21 ; COc1cnc2cc(C3CCN(c4c(C)cccc4F)CC3)c(=O)n(Cc3nccnc3C)c2n1 ; Cc1cnc2cc(C3CCN(c4ccnnc4C)CC3)c(=O)n(Cc3ncccc3C(F)(F)F)c2n1 ; Cc1cnc2cc(C3CCN(c4cncnc4C)CC3)c(=O)n(Cc3ncccc3C(F)(F)F)c2n1]

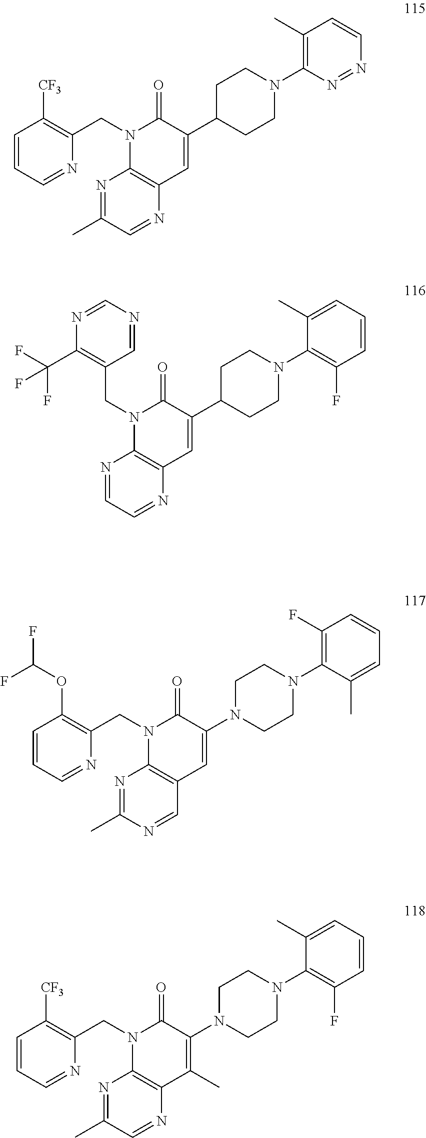 [SMILES: Cc1cnc2cc(C3CCN(c4nnccc4C)CC3)c(=O)n(Cc3ncccc3C(F)(F)F)c2n1 ; Cc1cccc(F)c1N1CCC(c2cc3nccnc3n(Cc3cncnc3C(F)(F)F)c2=O)CC1 ; Cc1ncc2cc(N3CCN(c4c(C)cccc4F)CC3)c(=O)n(Cc3ncccc3OC(F)F)c2n1 ; Cc1cnc2c(C)c(N3CCN(c4c(C)cccc4F)CC3)c(=O)n(Cc3ncccc3C(F)(F)F)c2n1]

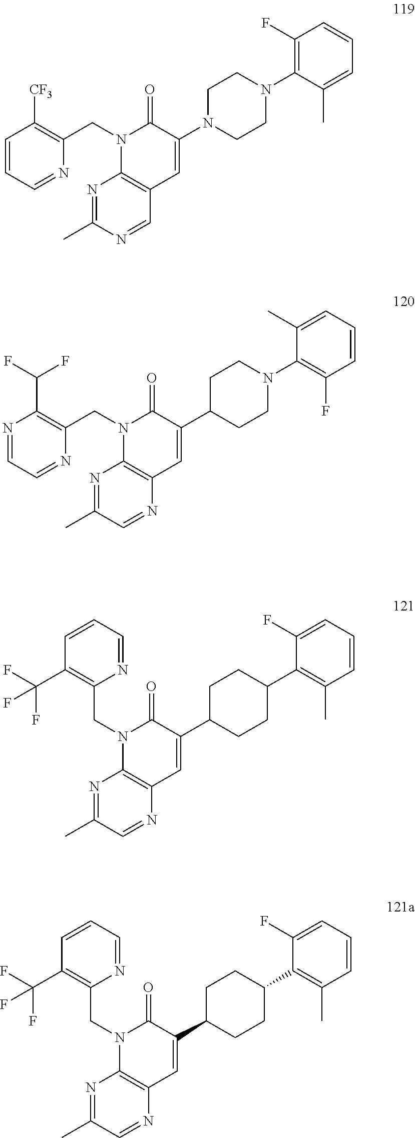 [SMILES: Cc1ncc2cc(N3CCN(c4c(C)cccc4F)CC3)c(=O)n(Cc3ncccc3C(F)(F)F)c2n1 ; Cc1cnc2cc(C3CCN(c4c(C)cccc4F)CC3)c(=O)n(Cc3nccnc3C(F)F)c2n1 ; Cc1cnc2cc(C3CCC(c4c(C)cccc4F)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3C(F)(F)F)c2n1]

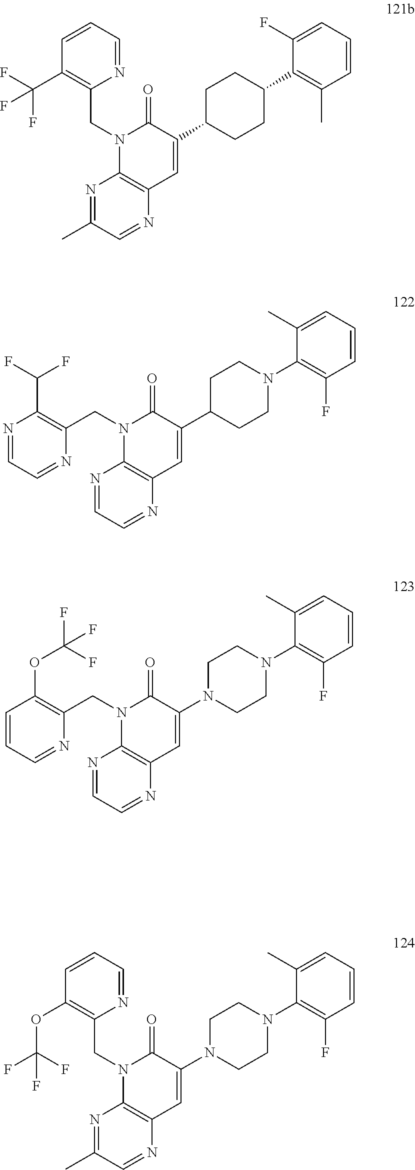 [SMILES: Cc1cnc2cc([C@H]3CC[C@@H](c4c(C)cccc4F)CC3)c(=O)n(Cc3ncccc3C(F)(F)F)c2n1 ; Cc1cccc(F)c1N1CCC(c2cc3nccnc3n(Cc3nccnc3C(F)F)c2=O)CC1 ; Cc1cccc(F)c1N1CCN(c2cc3nccnc3n(Cc3ncccc3OC(F)(F)F)c2=O)CC1 ; Cc1cnc2cc(N3CCN(c4c(C)cccc4F)CC3)c(=O)n(Cc3ncccc3OC(F)(F)F)c2n1]

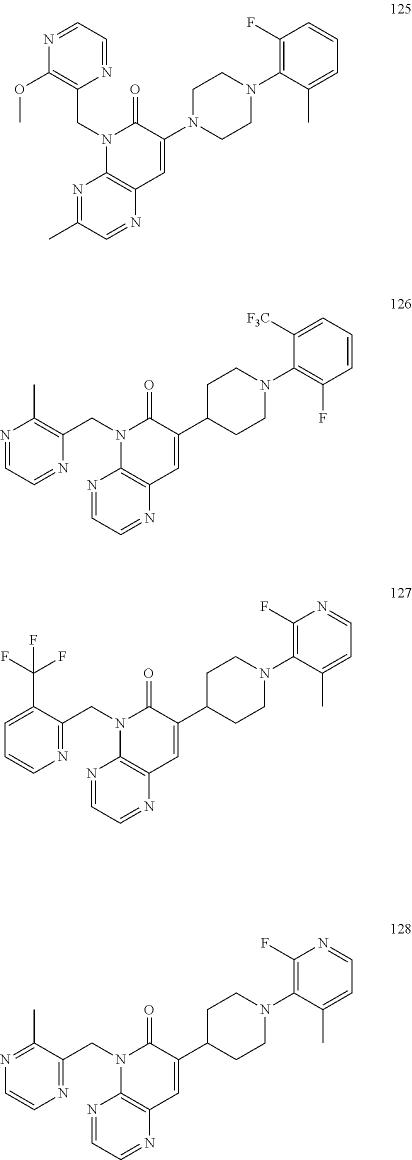 [SMILES: COc1nccnc1Cn1c(=O)c(N2CCN(c3c(C)cccc3F)CC2)cc2ncc(C)nc21 ; Cc1nccnc1Cn1c(=O)c(C2CCN(c3c(F)cccc3C(F)(F)F)CC2)cc2nccnc21 ; Cc1ccnc(F)c1N1CCC(c2cc3nccnc3n(Cc3ncccc3C(F)(F)F)c2=O)CC1 ; Cc1ccnc(F)c1N1CCC(c2cc3nccnc3n(Cc3nccnc3C)c2=O)CC1]

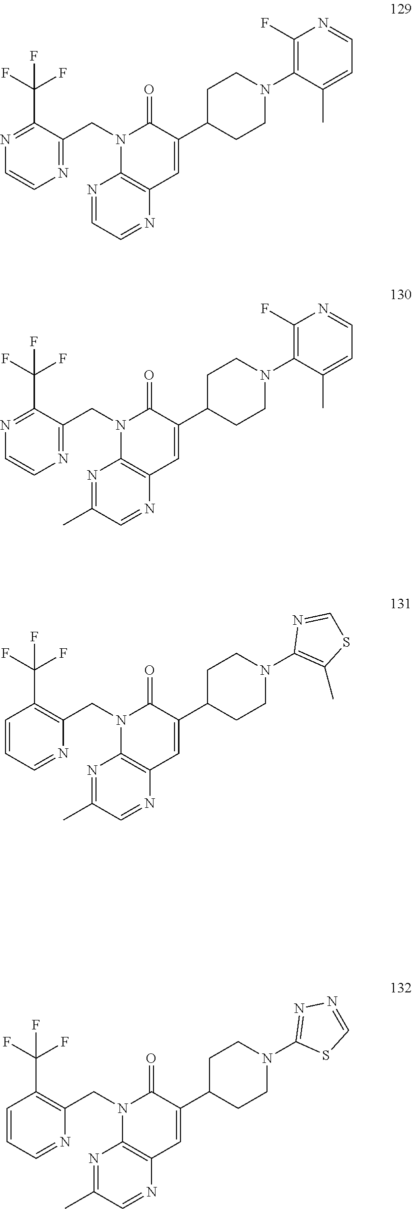 [SMILES: Cc1ccnc(F)c1N1CCC(c2cc3nccnc3n(Cc3nccnc3C(F)(F)F)c2=O)CC1 ; Cc1cnc2cc(C3CCN(c4c(C)ccnc4F)CC3)c(=O)n(Cc3nccnc3C(F)(F)F)c2n1 ; Cc1cnc2cc(C3CCN(c4ncsc4C)CC3)c(=O)n(Cc3ncccc3C(F)(F)F)c2n1 ; Cc1cnc2cc(C3CCN(c4nncs4)CC3)c(=O)n(Cc3ncccc3C(F)(F)F)c2n1]

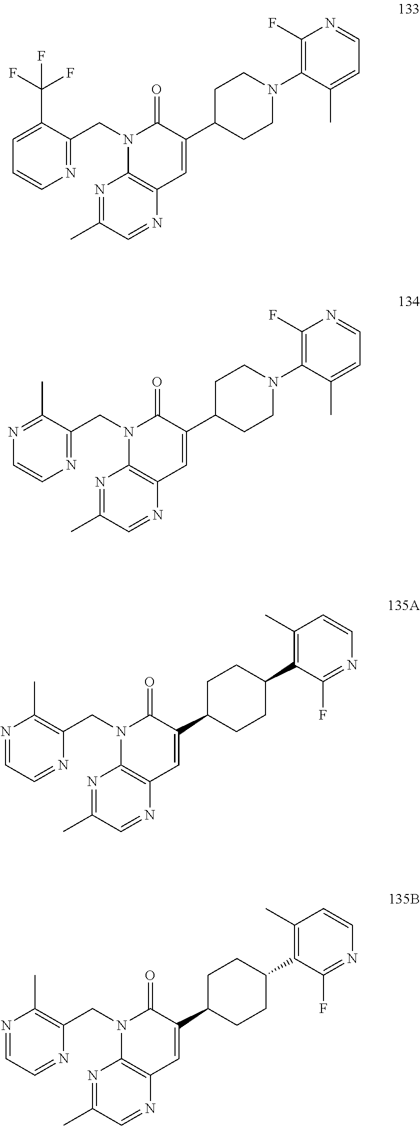 [SMILES: Cc1cnc2cc(C3CCN(c4c(C)ccnc4F)CC3)c(=O)n(Cc3ncccc3C(F)(F)F)c2n1 ; Cc1cnc2cc(C3CCN(c4c(C)ccnc4F)CC3)c(=O)n(Cc3nccnc3C)c2n1 ; Cc1cnc2cc([C@H]3CC[C@@H](c4c(C)ccnc4F)CC3)c(=O)n(Cc3nccnc3C)c2n1 ; Cc1cnc2cc([C@H]3CC[C@H](c4c(C)ccnc4F)CC3)c(=O)n(Cc3nccnc3C)c2n1]

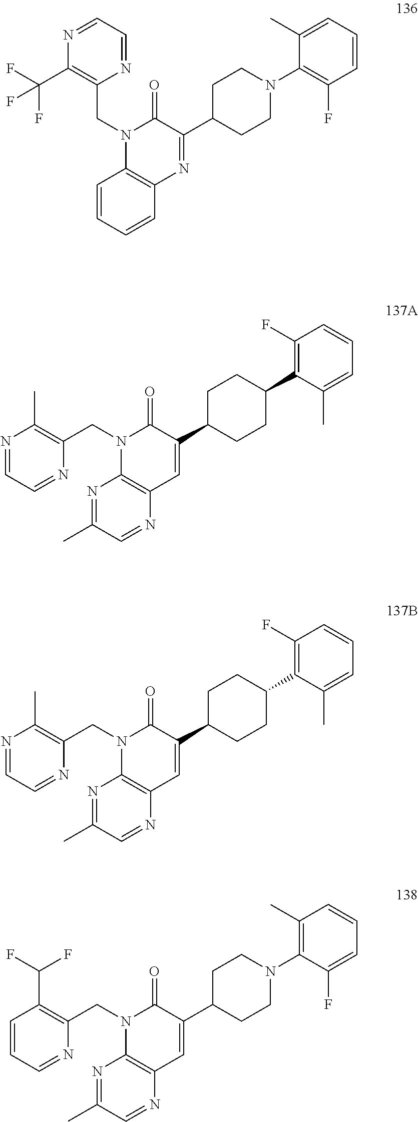 [SMILES: Cc1cccc(F)c1N1CCC(c2nc3ccccc3n(Cc3nccnc3C(F)(F)F)c2=O)CC1 ; Cc1cnc2cc([C@H]3CC[C@@H](c4c(C)cccc4F)CC3)c(=O)n(Cc3nccnc3C)c2n1 ; Cc1cnc2cc([C@H]3CC[C@H](c4c(C)cccc4F)CC3)c(=O)n(Cc3nccnc3C)c2n1 ; Cc1cnc2cc(C3CCN(c4c(C)cccc4F)CC3)c(=O)n(Cc3ncccc3C(F)F)c2n1]

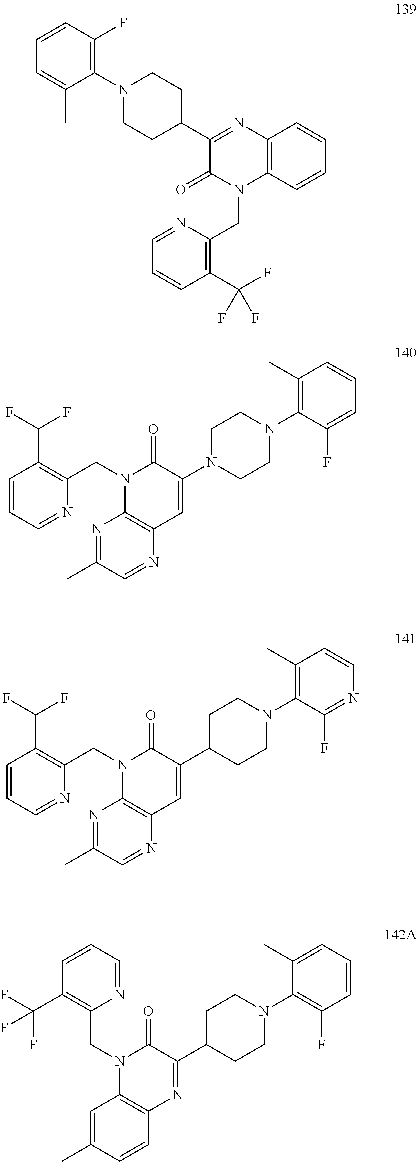

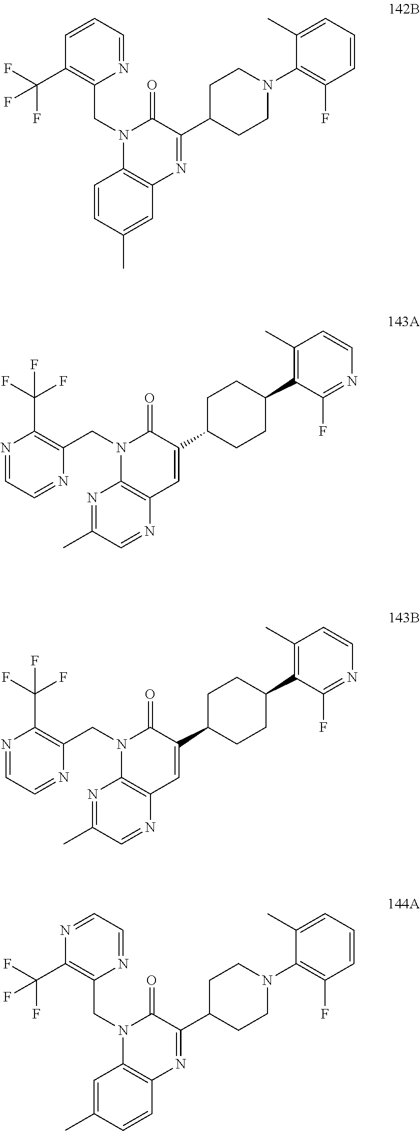 [SMILES: Cc1ccc2c(c1)nc(C1CCN(c3c(C)cccc3F)CC1)c(=O)n2Cc1ncccc1C(F)(F)F ; Cc1cnc2cc([C@H]3CC[C@H](c4c(C)ccnc4F)CC3)c(=O)n(Cc3nccnc3C(F)(F)F)c2n1 ; Cc1cnc2cc([C@H]3CC[C@@H](c4c(C)ccnc4F)CC3)c(=O)n(Cc3nccnc3C(F)(F)F)c2n1 ; Cc1ccc2nc(C3CCN(c4c(C)cccc4F)CC3)c(=O)n(Cc3nccnc3C(F)(F)F)c2c1]

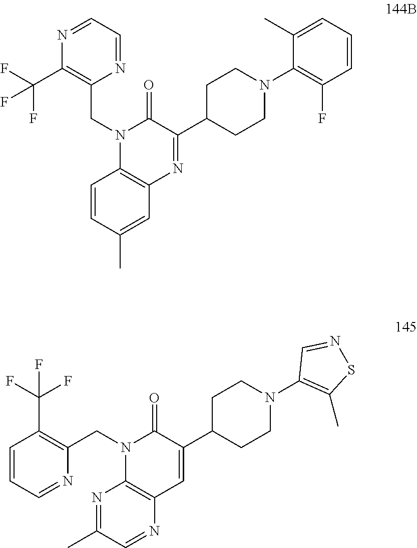 [SMILES: Cc1ccc2c(c1)nc(C1CCN(c3c(C)cccc3F)CC1)c(=O)n2Cc1nccnc1C(F)(F)F ; Cc1cnc2cc(C3CCN(c4cnsc4C)CC3)c(=O)n(Cc3ncccc3C(F)(F)F)c2n1]

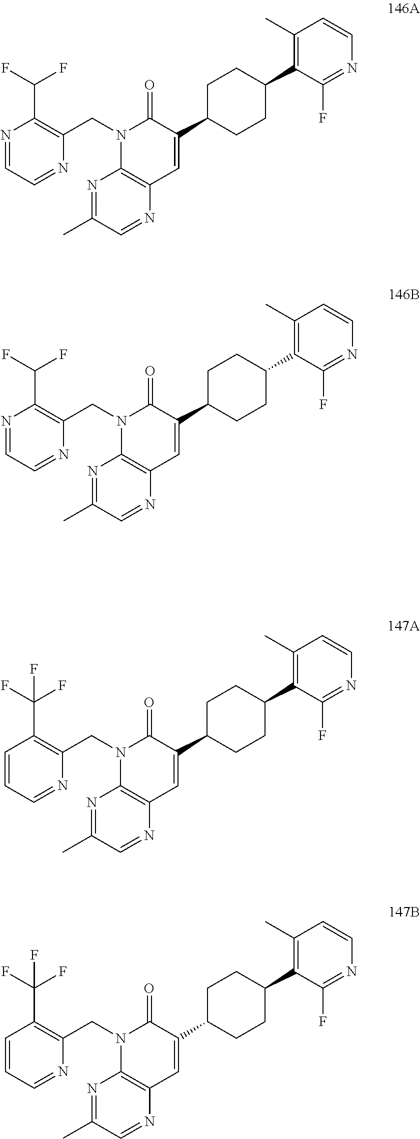 [SMILES: Cc1cnc2cc([C@H]3CC[C@@H](c4c(C)ccnc4F)CC3)c(=O)n(Cc3nccnc3C(F)F)c2n1 ; Cc1cnc2cc([C@H]3CC[C@H](c4c(C)ccnc4F)CC3)c(=O)n(Cc3nccnc3C(F)F)c2n1 ; Cc1cnc2cc([C@H]3CC[C@@H](c4c(C)ccnc4F)CC3)c(=O)n(Cc3ncccc3C(F)(F)F)c2n1 ; Cc1cnc2cc([C@H]3CC[C@H](c4c(C)ccnc4F)CC3)c(=O)n(Cc3ncccc3C(F)(F)F)c2n1]

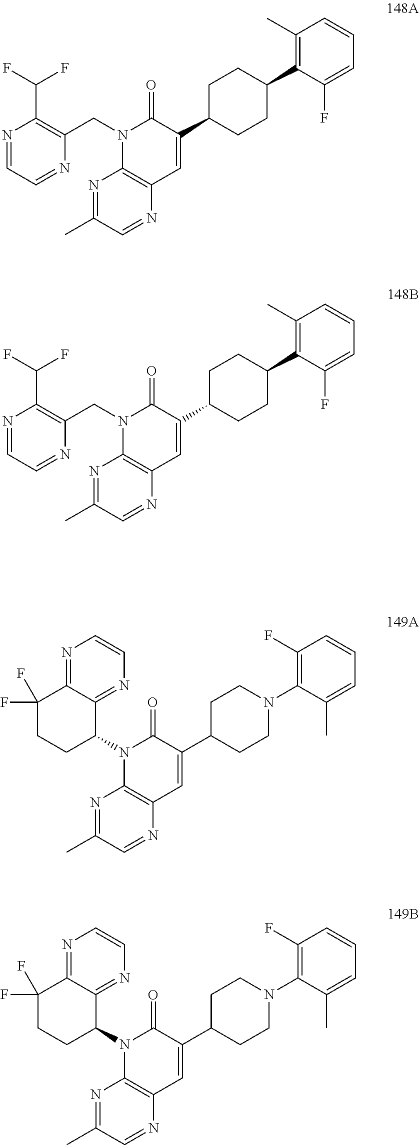 [SMILES: Cc1cnc2cc([C@H]3CC[C@@H](c4c(C)cccc4F)CC3)c(=O)n(Cc3nccnc3C(F)F)c2n1 ; Cc1cnc2cc([C@H]3CC[C@H](c4c(C)cccc4F)CC3)c(=O)n(Cc3nccnc3C(F)F)c2n1 ; Cc1cnc2cc(C3CCN(c4c(C)cccc4F)CC3)c(=O)n([C@@H]3CCC(F)(F)c4nccnc43)c2n1 ; Cc1cnc2cc(C3CCN(c4c(C)cccc4F)CC3)c(=O)n([C@H]3CCC(F)(F)c4nccnc43)c2n1]

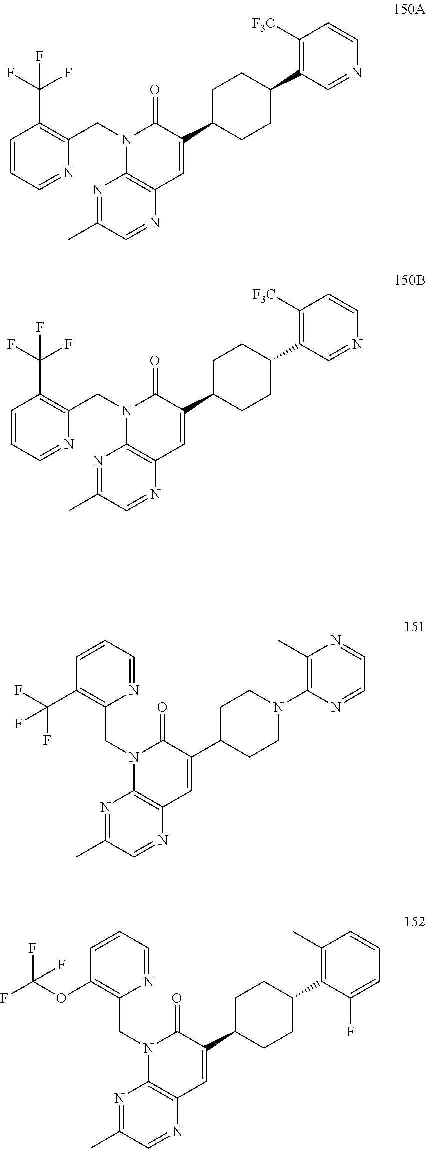 [SMILES: Cc1cnc2cc([C@H]3CC[C@@H](c4cnccc4C(F)(F)F)CC3)c(=O)n(Cc3ncccc3C(F)(F)F)c2n1 ; Cc1cnc2cc([C@H]3CC[C@H](c4cnccc4C(F)(F)F)CC3)c(=O)n(Cc3ncccc3C(F)(F)F)c2n1 ; Cc1cnc2cc(C3CCN(c4nccnc4C)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3OC(F)(F)F)c2n1]

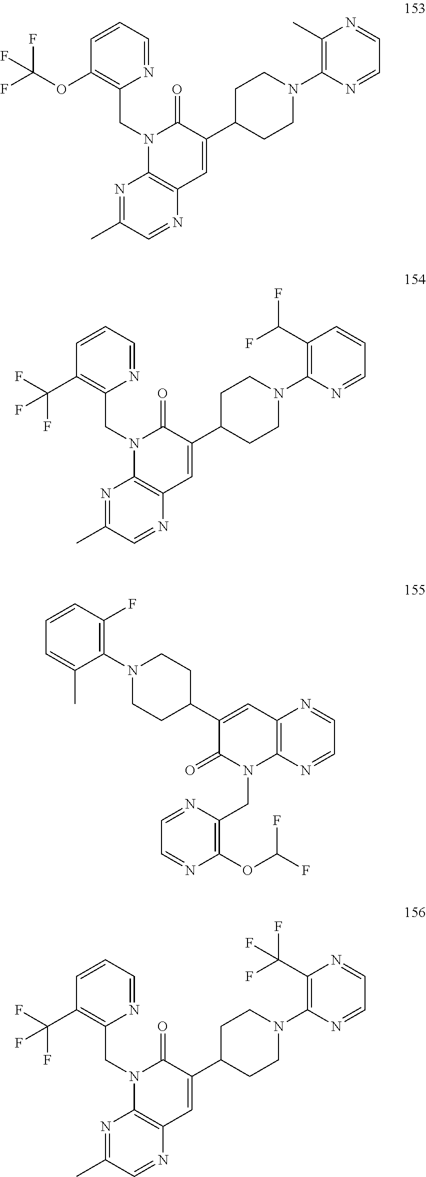 [SMILES: Cc1cnc2cc(C3CCN(c4nccnc4C)CC3)c(=O)n(Cc3ncccc3OC(F)(F)F)c2n1 ; Cc1cnc2cc(C3CCN(c4ncccc4C(F)F)CC3)c(=O)n(Cc3ncccc3C(F)(F)F)c2n1 ; Cc1cccc(F)c1N1CCC(c2cc3nccnc3n(Cc3nccnc3OC(F)F)c2=O)CC1 ; Cc1cnc2cc(C3CCN(c4nccnc4C(F)(F)F)CC3)c(=O)n(Cc3ncccc3C(F)(F)F)c2n1]

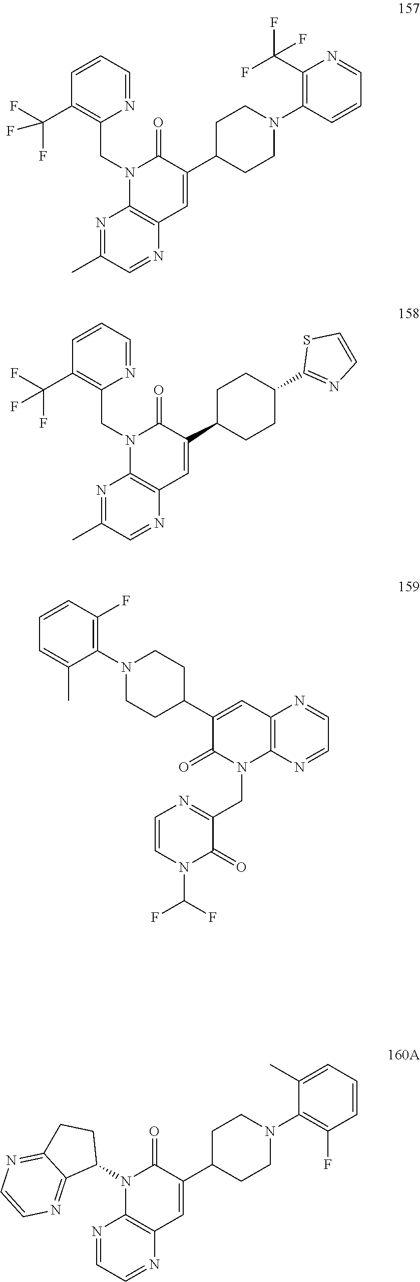 [SMILES: Cc1cnc2cc(C3CCN(c4cccnc4C(F)(F)F)CC3)c(=O)n(Cc3ncccc3C(F)(F)F)c2n1 ; Cc1cnc2cc([C@H]3CC[C@H](c4nccs4)CC3)c(=O)n(Cc3ncccc3C(F)(F)F)c2n1 ; Cc1cccc(F)c1N1CCC(c2cc3nccnc3n(Cc3nccn(C(F)F)c3=O)c2=O)CC1 ; Cc1cccc(F)c1N1CCC(c2cc3nccnc3n([C@H]3CCc4nccnc43)c2=O)CC1]

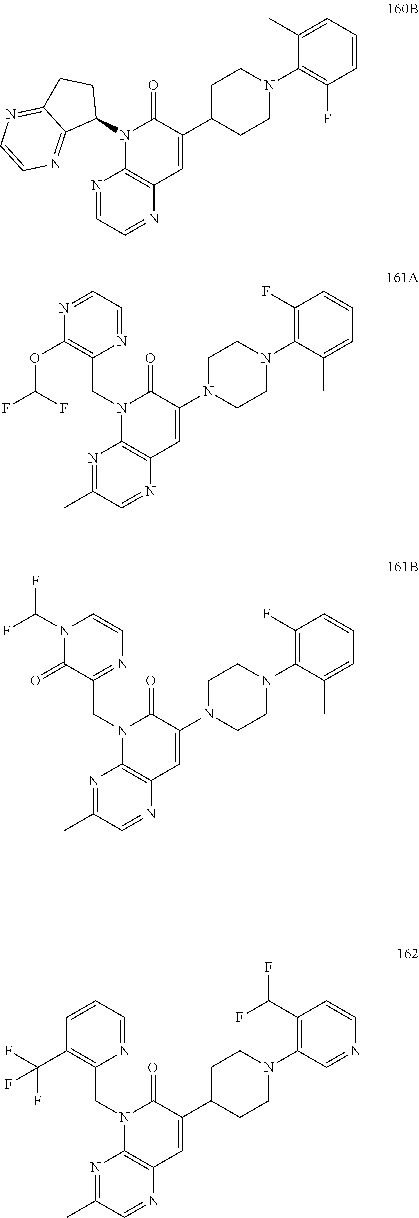 [SMILES: Cc1cccc(F)c1N1CCC(c2cc3nccnc3n([C@@H]3CCc4nccnc43)c2=O)CC1 ; Cc1cnc2cc(N3CCN(c4c(C)cccc4F)CC3)c(=O)n(Cc3nccnc3OC(F)F)c2n1 ; Cc1cnc2cc(N3CCN(c4c(C)cccc4F)CC3)c(=O)n(Cc3nccn(C(F)F)c3=O)c2n1 ; Cc1cnc2cc(C3CCN(c4cnccc4C(F)F)CC3)c(=O)n(Cc3ncccc3C(F)(F)F)c2n1]

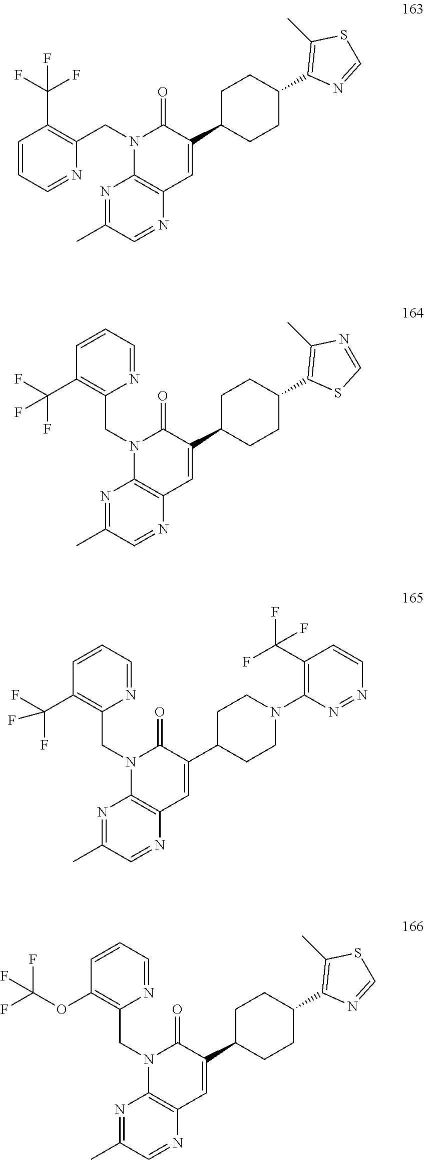 [SMILES: Cc1cnc2cc([C@H]3CC[C@H](c4ncsc4C)CC3)c(=O)n(Cc3ncccc3C(F)(F)F)c2n1 ; Cc1cnc2cc([C@H]3CC[C@H](c4scnc4C)CC3)c(=O)n(Cc3ncccc3C(F)(F)F)c2n1 ; Cc1cnc2cc(C3CCN(c4nnccc4C(F)(F)F)CC3)c(=O)n(Cc3ncccc3C(F)(F)F)c2n1 ; Cc1cnc2cc([C@H]3CC[C@H](c4ncsc4C)CC3)c(=O)n(Cc3ncccc3OC(F)(F)F)c2n1]

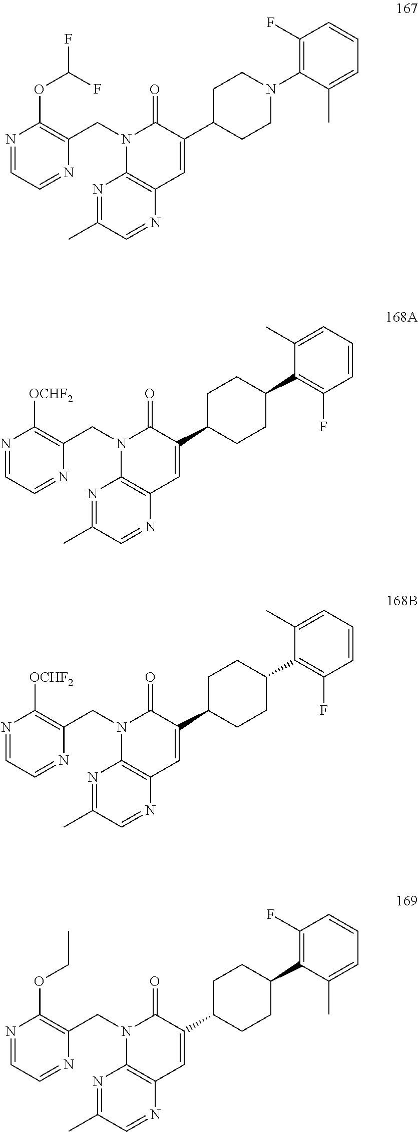 [SMILES: Cc1cnc2cc(C3CCN(c4c(C)cccc4F)CC3)c(=O)n(Cc3nccnc3OC(F)F)c2n1 ; Cc1cnc2cc([C@H]3CC[C@@H](c4c(C)cccc4F)CC3)c(=O)n(Cc3nccnc3OC(F)F)c2n1 ; Cc1cnc2cc([C@H]3CC[C@H](c4c(C)cccc4F)CC3)c(=O)n(Cc3nccnc3OC(F)F)c2n1 ; CCOc1nccnc1Cn1c(=O)c([C@H]2CC[C@H](c3c(C)cccc3F)CC2)cc2ncc(C)nc21]

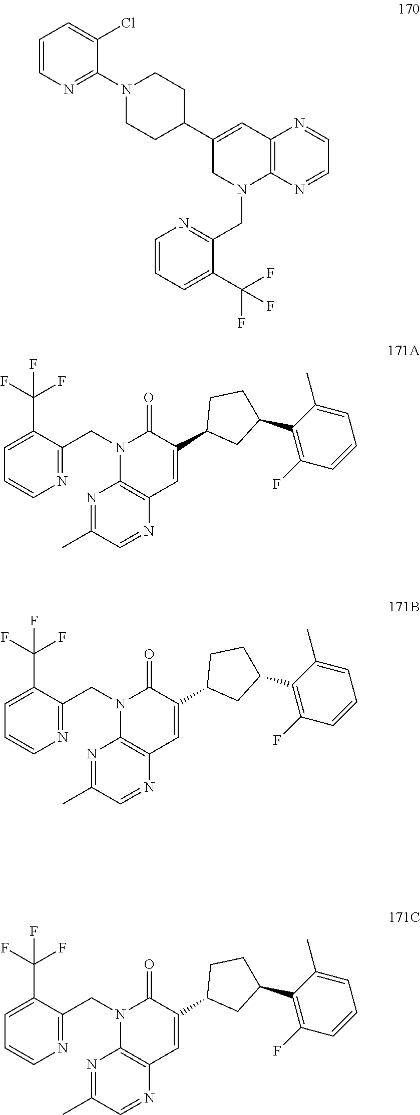 [SMILES: FC(F)(F)c1cccnc1CN1CC(C2CCN(c3ncccc3Cl)CC2)=Cc2nccnc21 ; Cc1cnc2cc([C@H]3CC[C@@H](c4c(C)cccc4F)C3)c(=O)n(Cc3ncccc3C(F)(F)F)c2n1 ; Cc1cnc2cc([C@@H]3CC[C@H](c4c(C)cccc4F)C3)c(=O)n(Cc3ncccc3C(F)(F)F)c2n1 ; Cc1cnc2cc([C@@H]3CC[C@@H](c4c(C)cccc4F)C3)c(=O)n(Cc3ncccc3C(F)(F)F)c2n1]

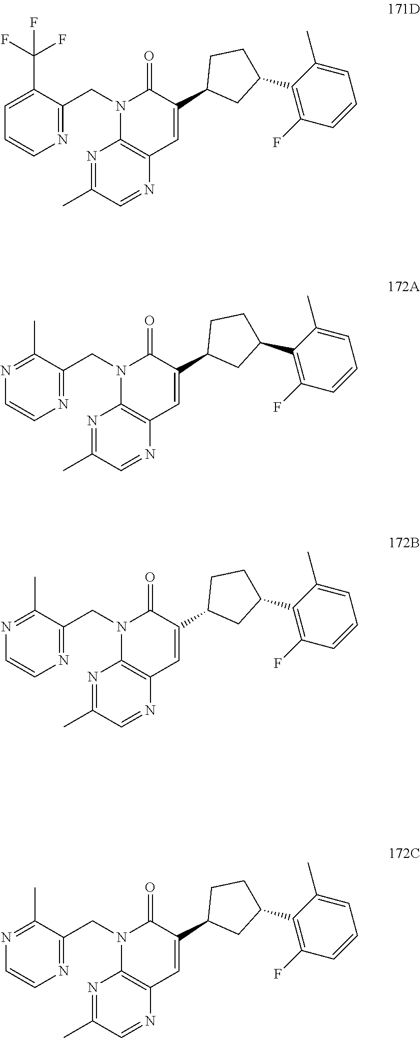 [SMILES: Cc1cnc2cc([C@H]3CC[C@H](c4c(C)cccc4F)C3)c(=O)n(Cc3ncccc3C(F)(F)F)c2n1 ; Cc1cnc2cc([C@H]3CC[C@@H](c4c(C)cccc4F)C3)c(=O)n(Cc3nccnc3C)c2n1 ; Cc1cnc2cc([C@@H]3CC[C@H](c4c(C)cccc4F)C3)c(=O)n(Cc3nccnc3C)c2n1 ; Cc1cnc2cc([C@H]3CC[C@H](c4c(C)cccc4F)C3)c(=O)n(Cc3nccnc3C)c2n1]

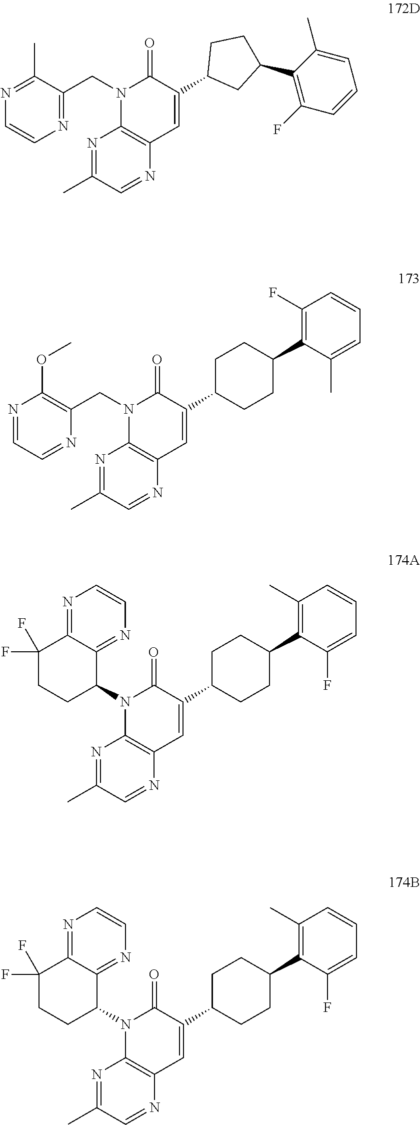 [SMILES: Cc1cnc2cc([C@@H]3CC[C@@H](c4c(C)cccc4F)C3)c(=O)n(Cc3nccnc3C)c2n1 ; COc1nccnc1Cn1c(=O)c([C@H]2CC[C@H](c3c(C)cccc3F)CC2)cc2ncc(C)nc21 ; Cc1cnc2cc([C@H]3CC[C@H](c4c(C)cccc4F)CC3)c(=O)n([C@H]3CCC(F)(F)c4nccnc43)c2n1 ; Cc1cnc2cc([C@H]3CC[C@H](c4c(C)cccc4F)CC3)c(=O)n([C@@H]3CCC(F)(F)c4nccnc43)c2n1]

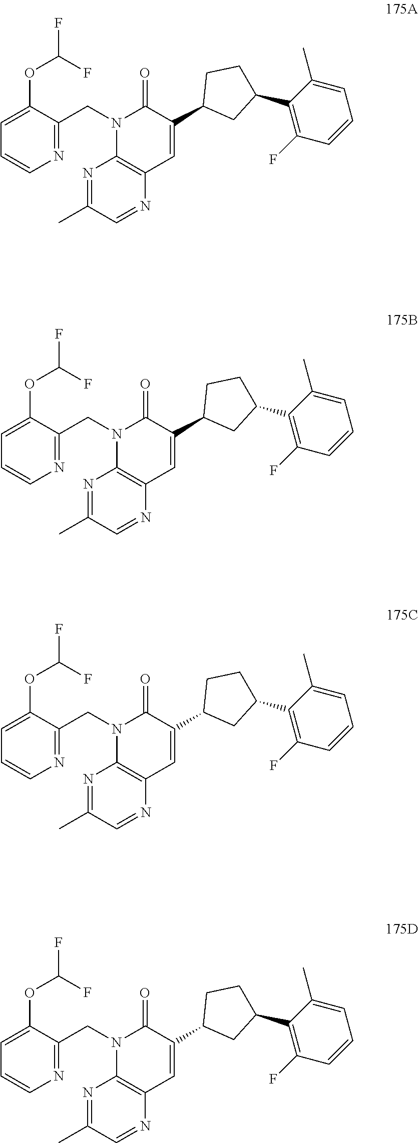 [SMILES: Cc1cnc2cc([C@H]3CC[C@@H](c4c(C)cccc4F)C3)c(=O)n(Cc3ncccc3OC(F)F)c2n1 ; Cc1cnc2cc([C@H]3CC[C@H](c4c(C)cccc4F)C3)c(=O)n(Cc3ncccc3OC(F)F)c2n1 ; Cc1cnc2cc([C@@H]3CC[C@H](c4c(C)cccc4F)C3)c(=O)n(Cc3ncccc3OC(F)F)c2n1 ; Cc1cnc2cc([C@@H]3CC[C@@H](c4c(C)cccc4F)C3)c(=O)n(Cc3ncccc3OC(F)F)c2n1]

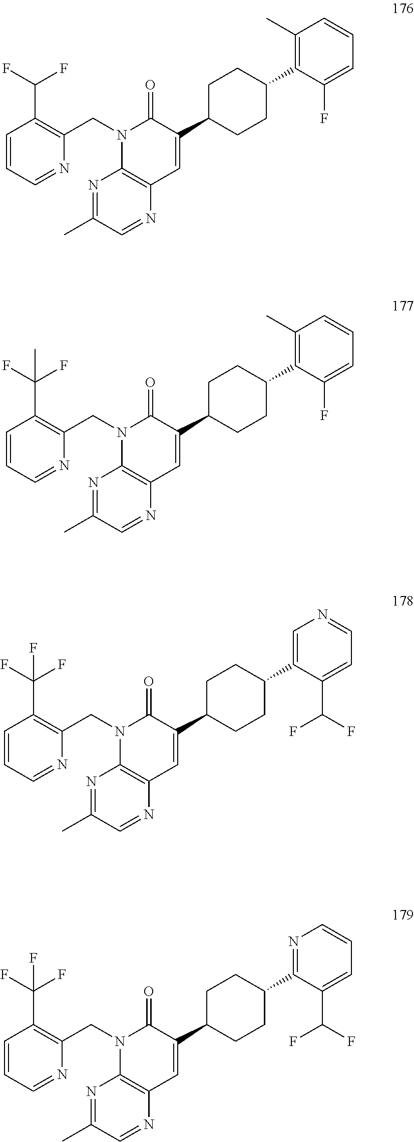 [SMILES: Cc1cnc2cc([C@H]3CC[C@H](c4c(C)cccc4F)CC3)c(=O)n(Cc3ncccc3C(F)F)c2n1 ; Cc1cnc2cc([C@H]3CC[C@H](c4c(C)cccc4F)CC3)c(=O)n(Cc3ncccc3C(C)(F)F)c2n1 ; Cc1cnc2cc([C@H]3CC[C@H](c4cnccc4C(F)F)CC3)c(=O)n(Cc3ncccc3C(F)(F)F)c2n1 ; Cc1cnc2cc([C@H]3CC[C@H](c4ncccc4C(F)F)CC3)c(=O)n(Cc3ncccc3C(F)(F)F)c2n1]

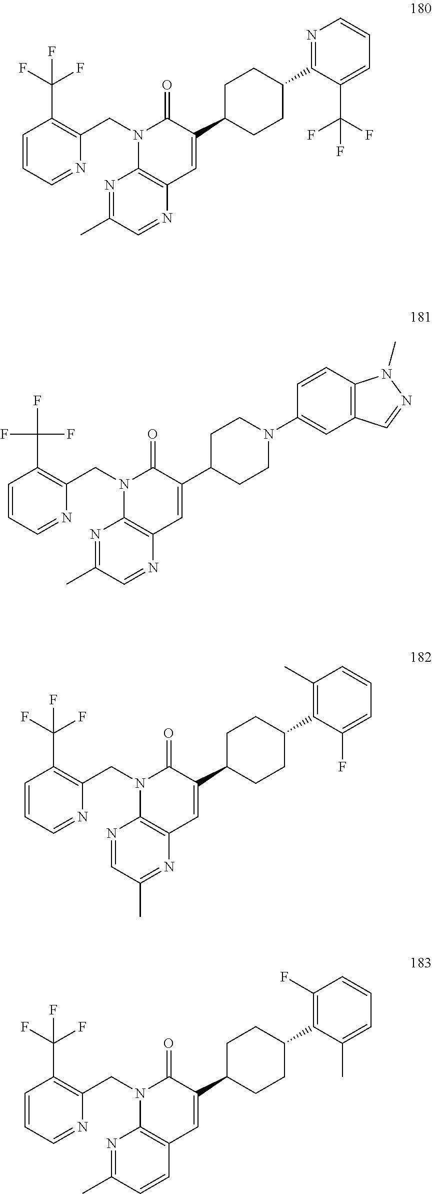 [SMILES: Cc1cnc2cc([C@H]3CC[C@H](c4ncccc4C(F)(F)F)CC3)c(=O)n(Cc3ncccc3C(F)(F)F)c2n1 ; Cc1cnc2cc(C3CCN(c4ccc5c(cnn5C)c4)CC3)c(=O)n(Cc3ncccc3C(F)(F)F)c2n1 ; Cc1cnc2c(cc([C@H]3CC[C@H](c4c(C)cccc4F)CC3)c(=O)n2Cc2ncccc2C(F)(F)F)n1 ; Cc1ccc2cc([C@H]3CC[C@H](c4c(C)cccc4F)CC3)c(=O)n(Cc3ncccc3C(F)(F)F)c2n1]

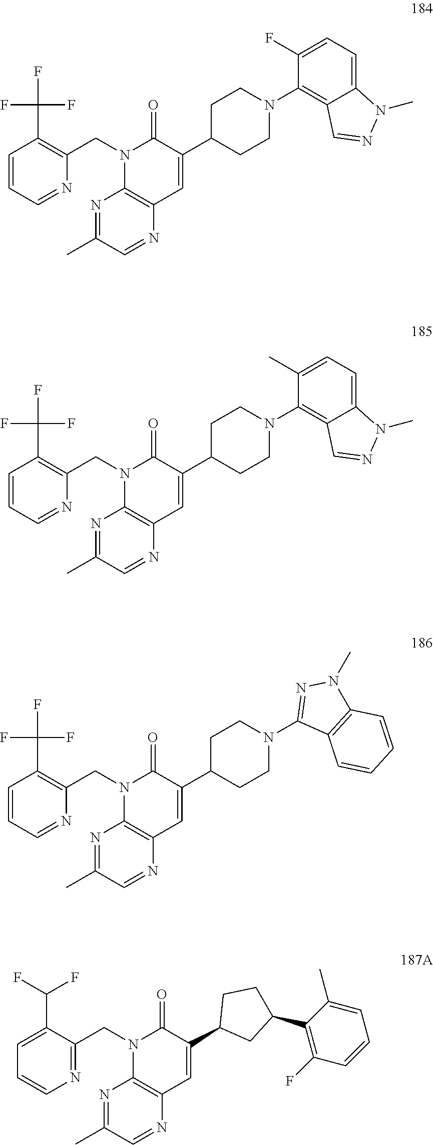 [SMILES: Cc1cnc2cc(C3CCN(c4c(F)ccc5c4cnn5C)CC3)c(=O)n(Cc3ncccc3C(F)(F)F)c2n1 ; Cc1cnc2cc(C3CCN(c4c(C)ccc5c4cnn5C)CC3)c(=O)n(Cc3ncccc3C(F)(F)F)c2n1 ; Cc1cnc2cc(C3CCN(c4nn(C)c5ccccc45)CC3)c(=O)n(Cc3ncccc3C(F)(F)F)c2n1 ; Cc1cnc2cc([C@H]3CC[C@@H](c4c(C)cccc4F)C3)c(=O)n(Cc3ncccc3C(F)F)c2n1]

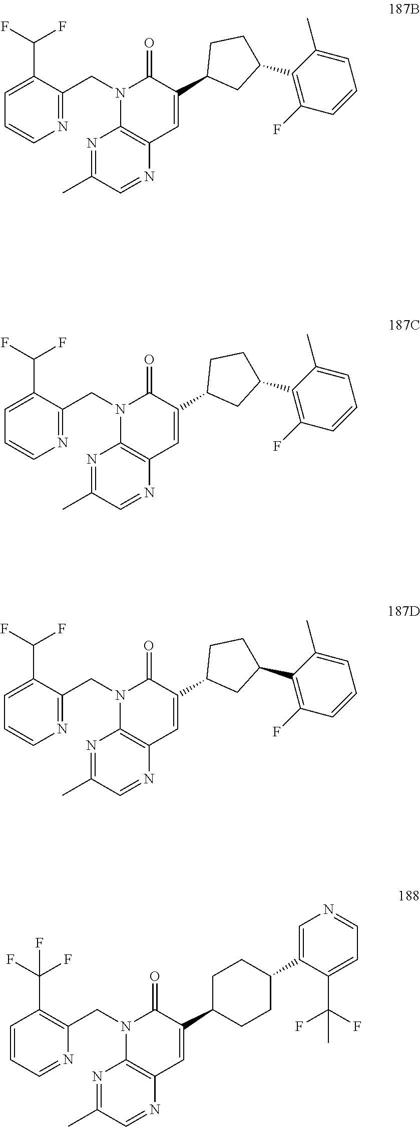 [SMILES: Cc1cnc2cc([C@H]3CC[C@H](c4c(C)cccc4F)C3)c(=O)n(Cc3ncccc3C(F)F)c2n1 ; Cc1cnc2cc([C@@H]3CC[C@H](c4c(C)cccc4F)C3)c(=O)n(Cc3ncccc3C(F)F)c2n1 ; Cc1cnc2cc([C@@H]3CC[C@@H](c4c(C)cccc4F)C3)c(=O)n(Cc3ncccc3C(F)F)c2n1 ; Cc1cnc2cc([C@H]3CC[C@H](c4cnccc4C(C)(F)F)CC3)c(=O)n(Cc3ncccc3C(F)(F)F)c2n1]

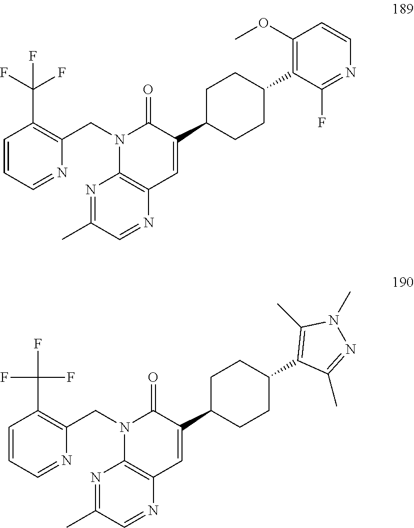 [SMILES: COc1ccnc(F)c1[C@H]1CC[C@H](c2cc3ncc(C)nc3n(Cc3ncccc3C(F)(F)F)c2=O)CC1 ; Cc1cnc2cc([C@H]3CC[C@H](c4c(C)nn(C)c4C)CC3)c(=O)n(Cc3ncccc3C(F)(F)F)c2n1]

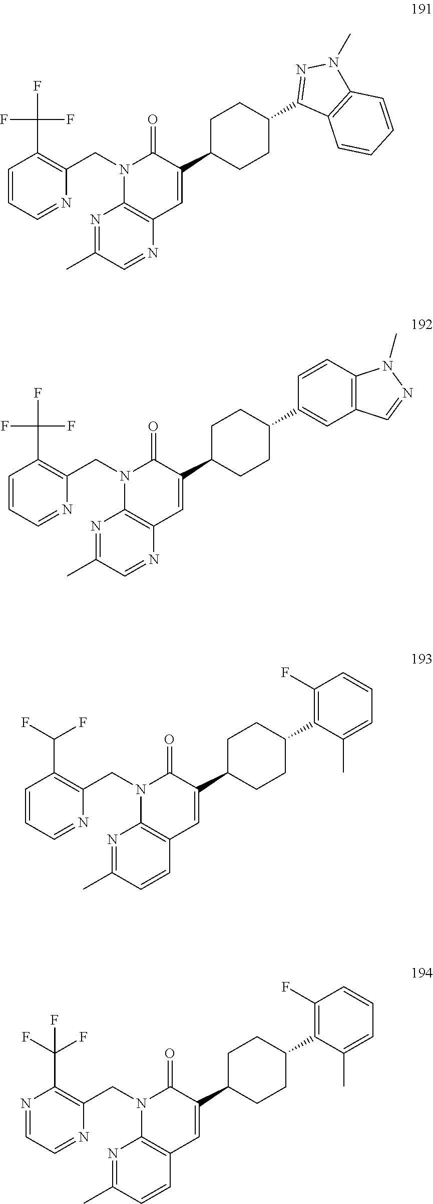 [SMILES: Cc1cnc2cc([C@H]3CC[C@H](c4nn(C)c5ccccc54)CC3)c(=O)n(Cc3ncccc3C(F)(F)F)c2n1 ; Cc1cnc2cc([C@H]3CC[C@H](c4ccc5c(cnn5C)c4)CC3)c(=O)n(Cc3ncccc3C(F)(F)F)c2n1 ; Cc1ccc2cc([C@H]3CC[C@H](c4c(C)cccc4F)CC3)c(=O)n(Cc3ncccc3C(F)F)c2n1 ; Cc1ccc2cc([C@H]3CC[C@H](c4c(C)cccc4F)CC3)c(=O)n(Cc3nccnc3C(F)(F)F)c2n1]

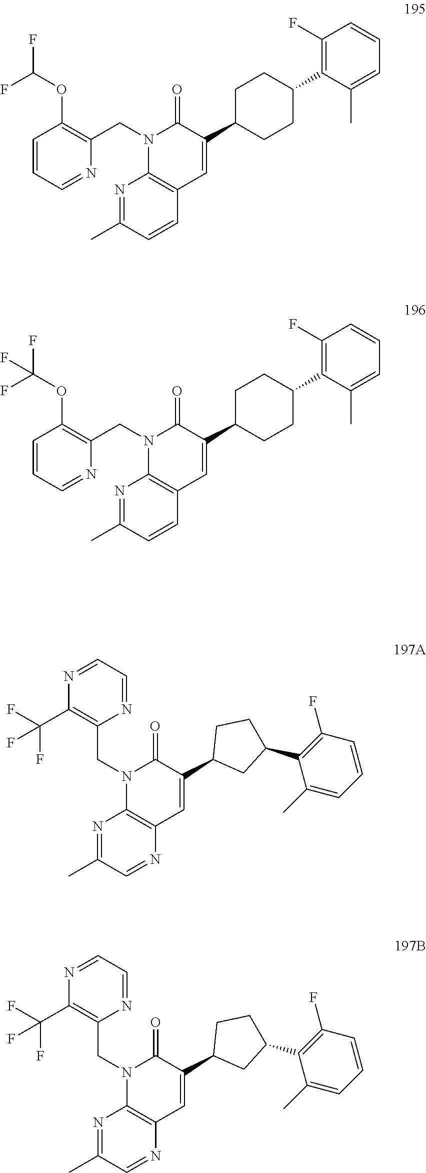 [SMILES: Cc1ccc2cc([C@H]3CC[C@H](c4c(C)cccc4F)CC3)c(=O)n(Cc3ncccc3OC(F)F)c2n1 ; Cc1ccc2cc([C@H]3CC[C@H](c4c(C)cccc4F)CC3)c(=O)n(Cc3ncccc3OC(F)(F)F)c2n1 ; Cc1cnc2cc([C@H]3CC[C@@H](c4c(C)cccc4F)C3)c(=O)n(Cc3nccnc3C(F)(F)F)c2n1 ; Cc1cnc2cc([C@H]3CC[C@H](c4c(C)cccc4F)C3)c(=O)n(Cc3nccnc3C(F)(F)F)c2n1]

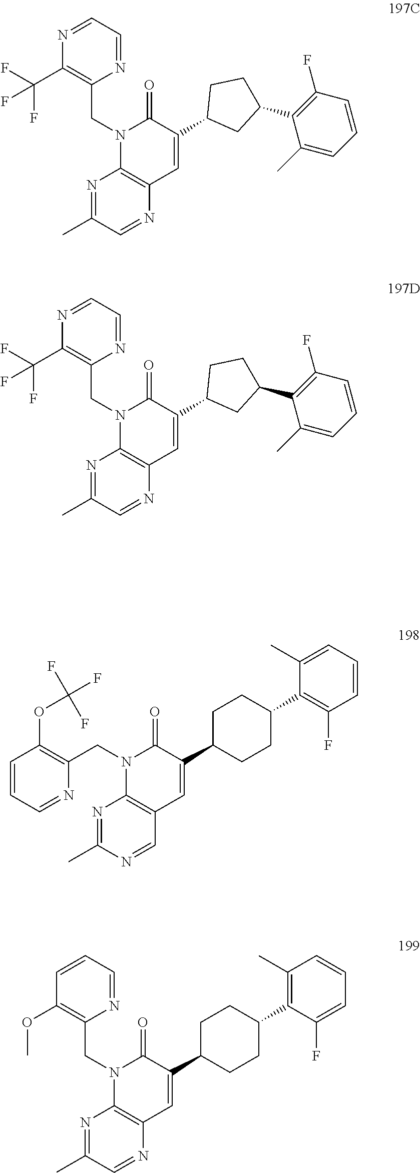 [SMILES: Cc1cnc2cc([C@@H]3CC[C@H](c4c(C)cccc4F)C3)c(=O)n(Cc3nccnc3C(F)(F)F)c2n1 ; Cc1cnc2cc([C@@H]3CC[C@@H](c4c(C)cccc4F)C3)c(=O)n(Cc3nccnc3C(F)(F)F)c2n1 ; Cc1ncc2cc([C@H]3CC[C@H](c4c(C)cccc4F)CC3)c(=O)n(Cc3ncccc3OC(F)(F)F)c2n1 ; COc1cccnc1Cn1c(=O)c([C@H]2CC[C@H](c3c(C)cccc3F)CC2)cc2ncc(C)nc21]

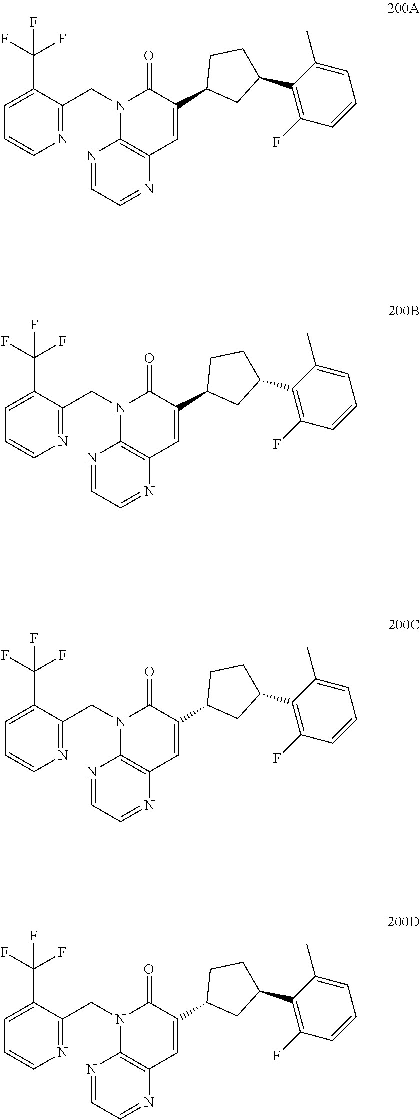 [SMILES: Cc1cccc(F)c1[C@@H]1CC[C@H](c2cc3nccnc3n(Cc3ncccc3C(F)(F)F)c2=O)C1 ; Cc1cccc(F)c1[C@H]1CC[C@H](c2cc3nccnc3n(Cc3ncccc3C(F)(F)F)c2=O)C1 ; Cc1cccc(F)c1[C@H]1CC[C@@H](c2cc3nccnc3n(Cc3ncccc3C(F)(F)F)c2=O)C1 ; Cc1cccc(F)c1[C@@H]1CC[C@@H](c2cc3nccnc3n(Cc3ncccc3C(F)(F)F)c2=O)C1]

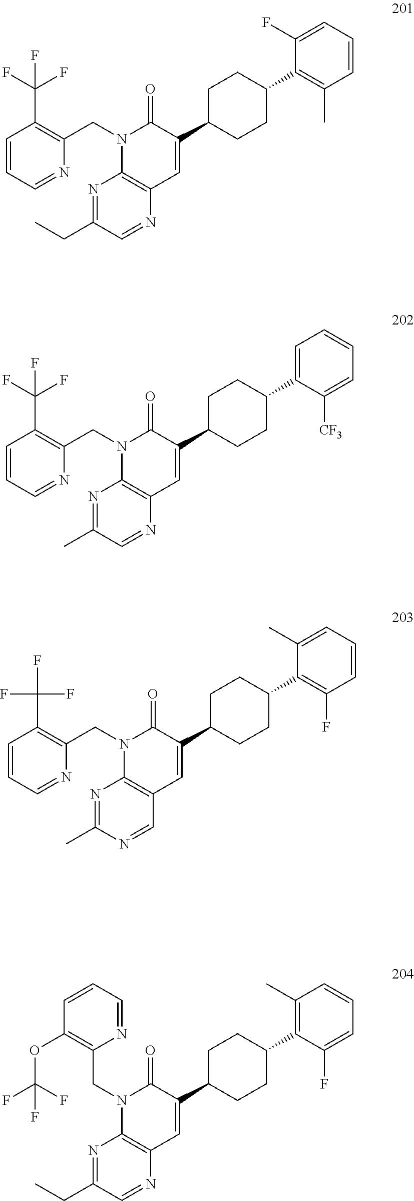 [SMILES: CCc1cnc2cc([C@H]3CC[C@H](c4c(C)cccc4F)CC3)c(=O)n(Cc3ncccc3C(F)(F)F)c2n1 ; Cc1cnc2cc([C@H]3CC[C@H](c4ccccc4C(F)(F)F)CC3)c(=O)n(Cc3ncccc3C(F)(F)F)c2n1 ; Cc1ncc2cc([C@H]3CC[C@H](c4c(C)cccc4F)CC3)c(=O)n(Cc3ncccc3C(F)(F)F)c2n1 ; CCc1cnc2cc([C@H]3CC[C@H](c4c(C)cccc4F)CC3)c(=O)n(Cc3ncccc3OC(F)(F)F)c2n1]

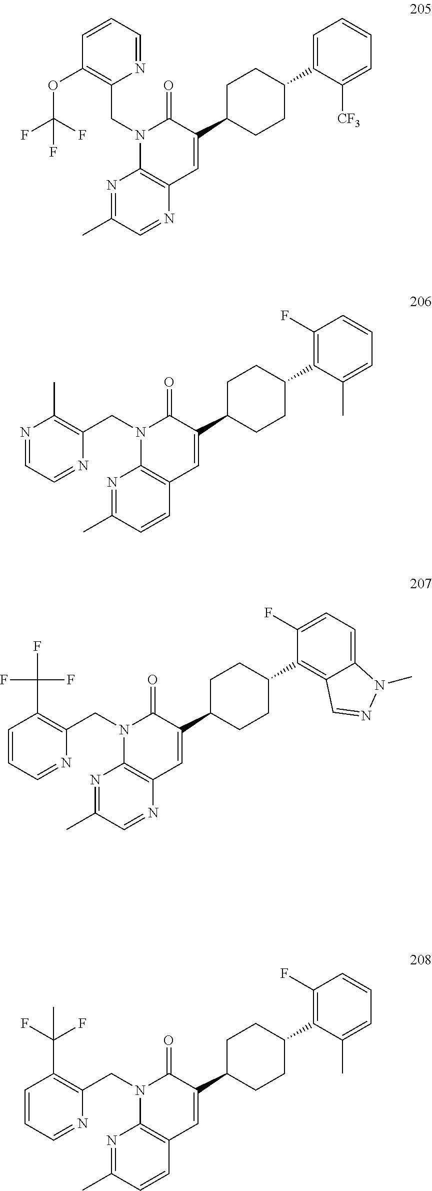 [SMILES: Cc1cnc2cc([C@H]3CC[C@H](c4ccccc4C(F)(F)F)CC3)c(=O)n(Cc3ncccc3OC(F)(F)F)c2n1 ; Cc1ccc2cc([C@H]3CC[C@H](c4c(C)cccc4F)CC3)c(=O)n(Cc3nccnc3C)c2n1 ; Cc1cnc2cc([C@H]3CC[C@H](c4c(F)ccc5c4cnn5C)CC3)c(=O)n(Cc3ncccc3C(F)(F)F)c2n1 ; Cc1ccc2cc([C@H]3CC[C@H](c4c(C)cccc4F)CC3)c(=O)n(Cc3ncccc3C(C)(F)F)c2n1]

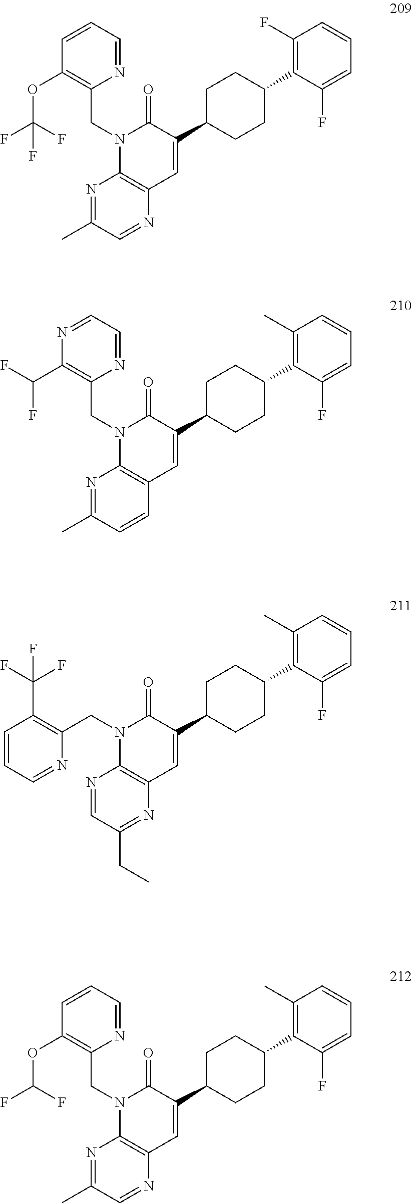 [SMILES: Cc1cnc2cc([C@H]3CC[C@H](c4c(F)cccc4F)CC3)c(=O)n(Cc3ncccc3OC(F)(F)F)c2n1 ; Cc1ccc2cc([C@H]3CC[C@H](c4c(C)cccc4F)CC3)c(=O)n(Cc3nccnc3C(F)F)c2n1 ; CCc1cnc2c(cc([C@H]3CC[C@H](c4c(C)cccc4F)CC3)c(=O)n2Cc2ncccc2C(F)(F)F)n1 ; Cc1cnc2cc([C@H]3CC[C@H](c4c(C)cccc4F)CC3)c(=O)n(Cc3ncccc3OC(F)F)c2n1]

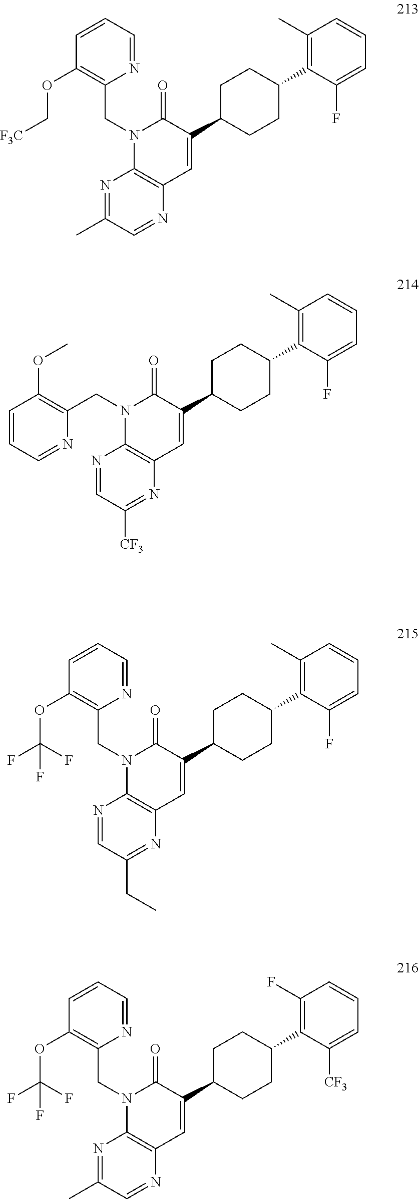 [SMILES: Cc1cnc2cc([C@H]3CC[C@H](c4c(C)cccc4F)CC3)c(=O)n(Cc3ncccc3OCC(F)(F)F)c2n1 ; COc1cccnc1Cn1c(=O)c([C@H]2CC[C@H](c3c(C)cccc3F)CC2)cc2nc(C(F)(F)F)cnc21 ; CCc1cnc2c(cc([C@H]3CC[C@H](c4c(C)cccc4F)CC3)c(=O)n2Cc2ncccc2OC(F)(F)F)n1 ; Cc1cnc2cc([C@H]3CC[C@H](c4c(F)cccc4C(F)(F)F)CC3)c(=O)n(Cc3ncccc3OC(F)(F)F)c2n1]

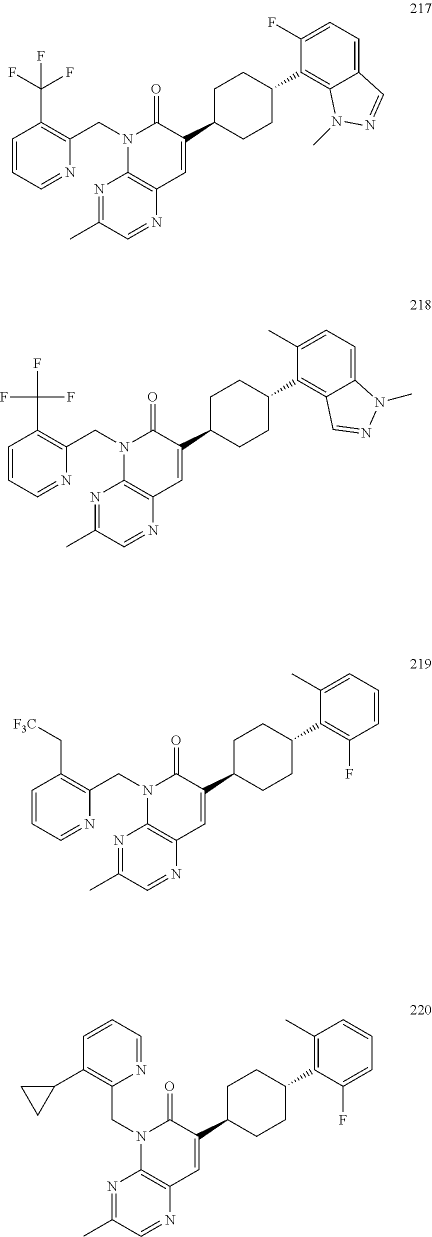 [SMILES: Cc1cnc2cc([C@H]3CC[C@H](c4c(F)ccc5cnn(C)c54)CC3)c(=O)n(Cc3ncccc3C(F)(F)F)c2n1 ; Cc1cnc2cc([C@H]3CC[C@H](c4c(C)ccc5c4cnn5C)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3CC(F)(F)F)c2n1 ; Cc1cnc2cc([C@H]3CC[C@H](c4c(C)cccc4F)CC3)c(=O)n(Cc3ncccc3C3CC3)c2n1]

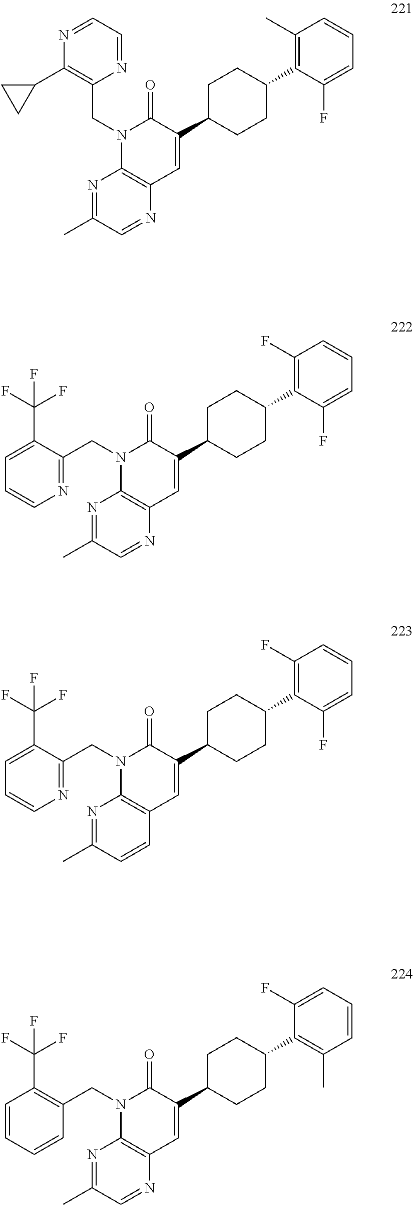 [SMILES: Cc1cnc2cc([C@H]3CC[C@H](c4c(C)cccc4F)CC3)c(=O)n(Cc3nccnc3C3CC3)c2n1 ; Cc1cnc2cc([C@H]3CC[C@H](c4c(F)cccc4F)CC3)c(=O)n(Cc3ncccc3C(F)(F)F)c2n1 ; Cc1ccc2cc([C@H]3CC[C@H](c4c(F)cccc4F)CC3)c(=O)n(Cc3ncccc3C(F)(F)F)c2n1 ; Cc1cnc2cc([C@H]3CC[C@H](c4c(C)cccc4F)CC3)c(=O)n(Cc3ccccc3C(F)(F)F)c2n1]

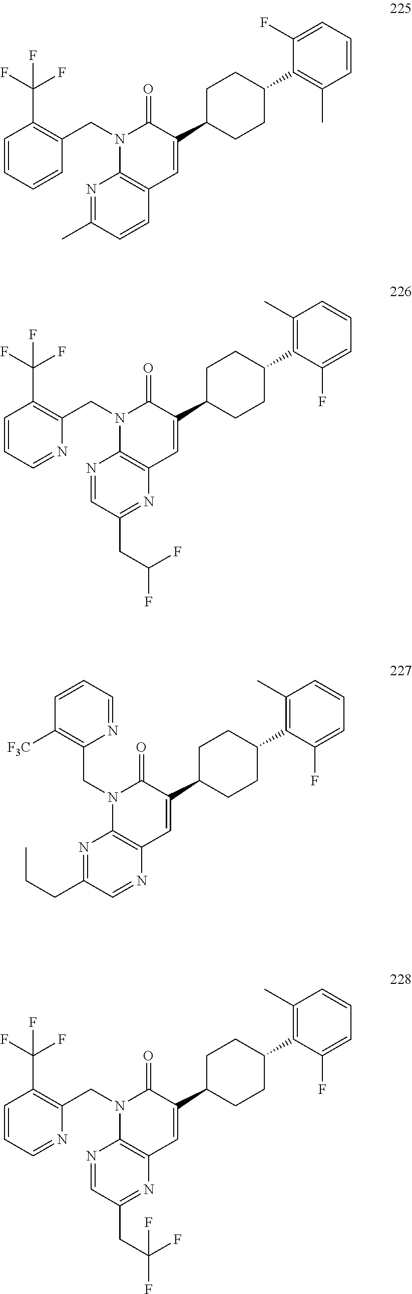 [SMILES: Cc1ccc2cc([C@H]3CC[C@H](c4c(C)cccc4F)CC3)c(=O)n(Cc3ccccc3C(F)(F)F)c2n1 ; Cc1cccc(F)c1[C@H]1CC[C@H](c2cc3nc(CC(F)F)cnc3n(Cc3ncccc3C(F)(F)F)c2=O)CC1 ; CCCc1cnc2cc([C@H]3CC[C@H](c4c(C)cccc4F)CC3)c(=O)n(Cc3ncccc3C(F)(F)F)c2n1 ; Cc1cccc(F)c1[C@H]1CC[C@H](c2cc3nc(CC(F)(F)F)cnc3n(Cc3ncccc3C(F)(F)F)c2=O)CC1]

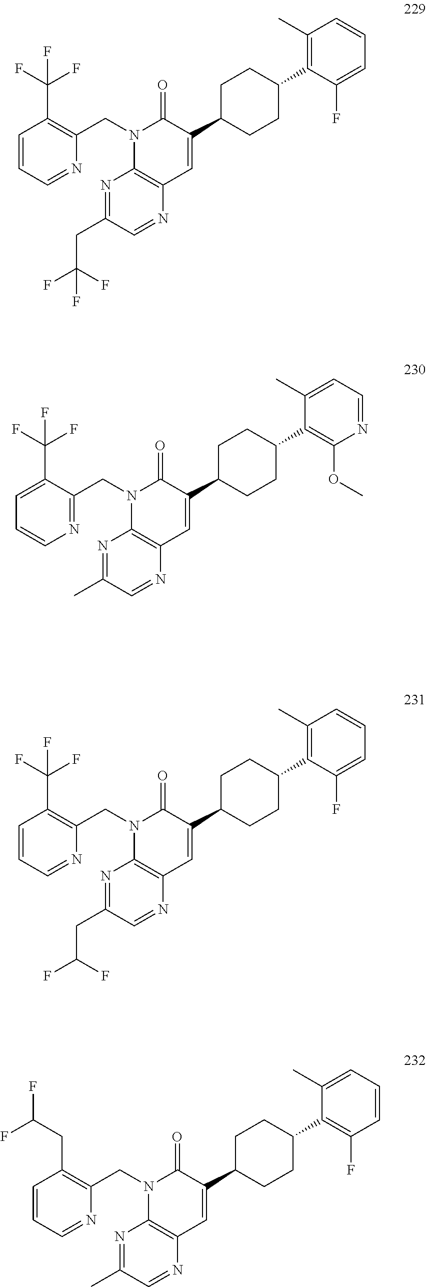 [SMILES: Cc1cccc(F)c1[C@H]1CC[C@H](c2cc3ncc(CC(F)(F)F)nc3n(Cc3ncccc3C(F)(F)F)c2=O)CC1 ; COc1nccc(C)c1[C@H]1CC[C@H](c2cc3ncc(C)nc3n(Cc3ncccc3C(F)(F)F)c2=O)CC1 ; Cc1cccc(F)c1[C@H]1CC[C@H](c2cc3ncc(CC(F)F)nc3n(Cc3ncccc3C(F)(F)F)c2=O)CC1 ; Cc1cnc2cc([C@H]3CC[C@H](c4c(C)cccc4F)CC3)c(=O)n(Cc3ncccc3CC(F)F)c2n1]

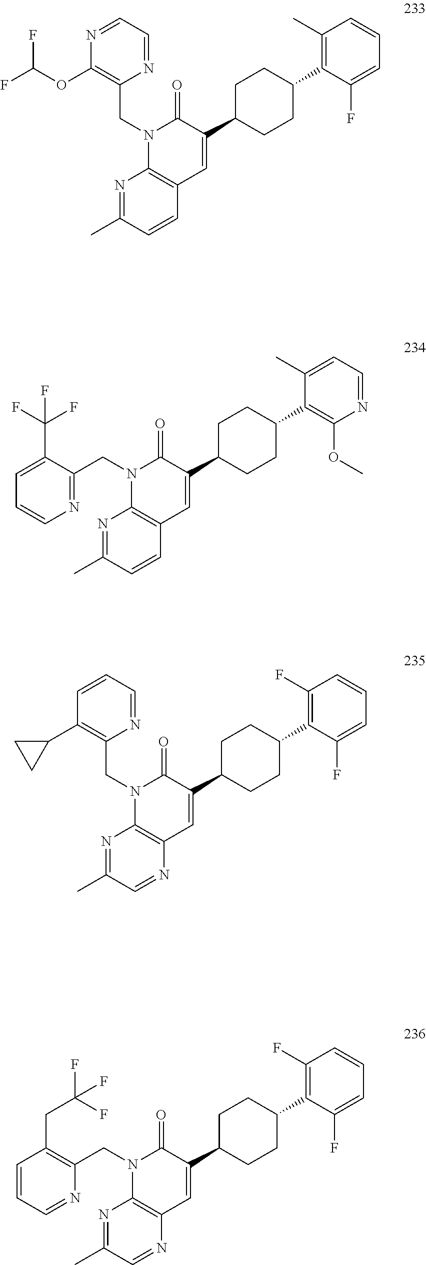 [SMILES: Cc1ccc2cc([C@H]3CC[C@H](c4c(C)cccc4F)CC3)c(=O)n(Cc3nccnc3OC(F)F)c2n1 ; COc1nccc(C)c1[C@H]1CC[C@H](c2cc3ccc(C)nc3n(Cc3ncccc3C(F)(F)F)c2=O)CC1 ; Cc1cnc2cc([C@H]3CC[C@H](c4c(F)cccc4F)CC3)c(=O)n(Cc3ncccc3C3CC3)c2n1 ; Cc1cnc2cc([C@H]3CC[C@H](c4c(F)cccc4F)CC3)c(=O)n(Cc3ncccc3CC(F)(F)F)c2n1]

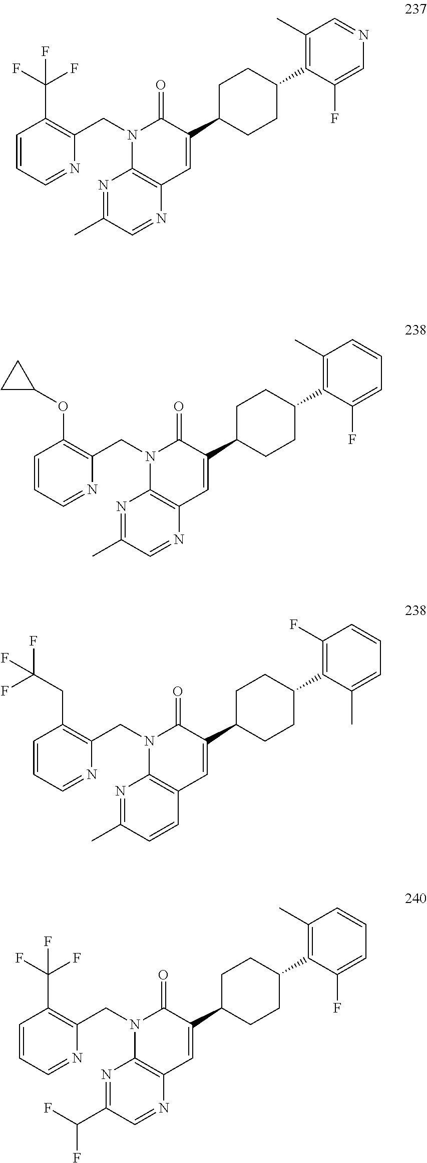 [SMILES: Cc1cnc2cc([C@H]3CC[C@H](c4c(C)cncc4F)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3OC3CC3)c2n1 ; Cc1ccc2cc([C@H]3CC[C@H](c4c(C)cccc4F)CC3)c(=O)n(Cc3ncccc3CC(F)(F)F)c2n1 ; Cc1cccc(F)c1[C@H]1CC[C@H](c2cc3ncc(C(F)F)nc3n(Cc3ncccc3C(F)(F)F)c2=O)CC1]

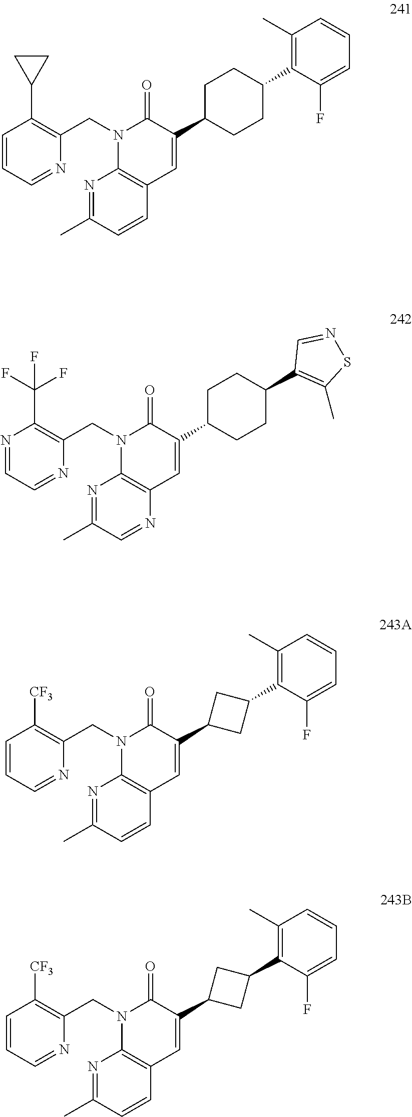 [SMILES: Cc1ccc2cc([C@H]3CC[C@H](c4c(C)cccc4F)CC3)c(=O)n(Cc3ncccc3C3CC3)c2n1 ; Cc1cnc2cc([C@H]3CC[C@H](c4cnsc4C)CC3)c(=O)n(Cc3nccnc3C(F)(F)F)c2n1 ; Cc1ccc2cc([C@H]3C[C@H](c4c(C)cccc4F)C3)c(=O)n(Cc3ncccc3C(F)(F)F)c2n1 ; Cc1ccc2cc([C@H]3C[C@@H](c4c(C)cccc4F)C3)c(=O)n(Cc3ncccc3C(F)(F)F)c2n1]

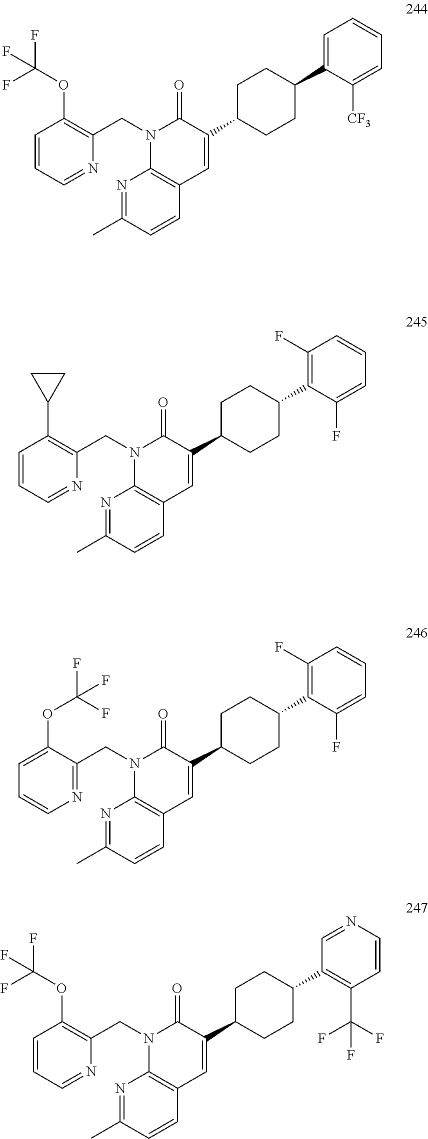 [SMILES: Cc1ccc2cc([C@H]3CC[C@H](c4ccccc4C(F)(F)F)CC3)c(=O)n(Cc3ncccc3OC(F)(F)F)c2n1 ; Cc1ccc2cc([C@H]3CC[C@H](c4c(F)cccc4F)CC3)c(=O)n(Cc3ncccc3C3CC3)c2n1 ; Cc1ccc2cc([C@H]3CC[C@H](c4c(F)cccc4F)CC3)c(=O)n(Cc3ncccc3OC(F)(F)F)c2n1 ; Cc1ccc2cc([C@H]3CC[C@H](c4cnccc4C(F)(F)F)CC3)c(=O)n(Cc3ncccc3OC(F)(F)F)c2n1]

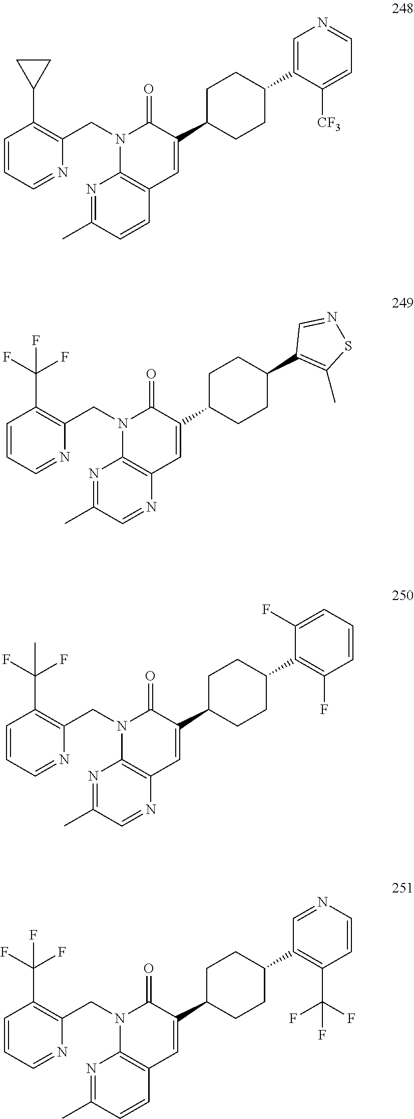 [SMILES: Cc1ccc2cc([C@H]3CC[C@H](c4cnccc4C(F)(F)F)CC3)c(=O)n(Cc3ncccc3C3CC3)c2n1 ; Cc1cnc2cc([C@H]3CC[C@H](c4cnsc4C)CC3)c(=O)n(Cc3ncccc3C(F)(F)F)c2n1 ; Cc1cnc2cc([C@H]3CC[C@H](c4c(F)cccc4F)CC3)c(=O)n(Cc3ncccc3C(C)(F)F)c2n1 ; Cc1ccc2cc([C@H]3CC[C@H](c4cnccc4C(F)(F)F)CC3)c(=O)n(Cc3ncccc3C(F)(F)F)c2n1]

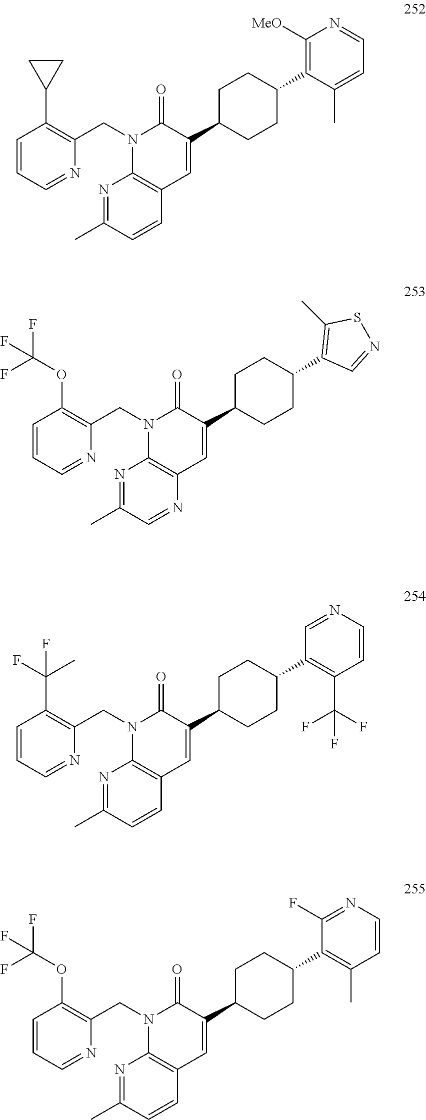 [SMILES: COc1nccc(C)c1[C@H]1CC[C@H](c2cc3ccc(C)nc3n(Cc3ncccc3C3CC3)c2=O)CC1 ; Cc1cnc2cc([C@H]3CC[C@H](c4cnsc4C)CC3)c(=O)n(Cc3ncccc3OC(F)(F)F)c2n1 ; Cc1ccc2cc([C@H]3CC[C@H](c4cnccc4C(F)(F)F)CC3)c(=O)n(Cc3ncccc3C(C)(F)F)c2n1 ; Cc1ccc2cc([C@H]3CC[C@H](c4c(C)ccnc4F)CC3)c(=O)n(Cc3ncccc3OC(F)(F)F)c2n1]

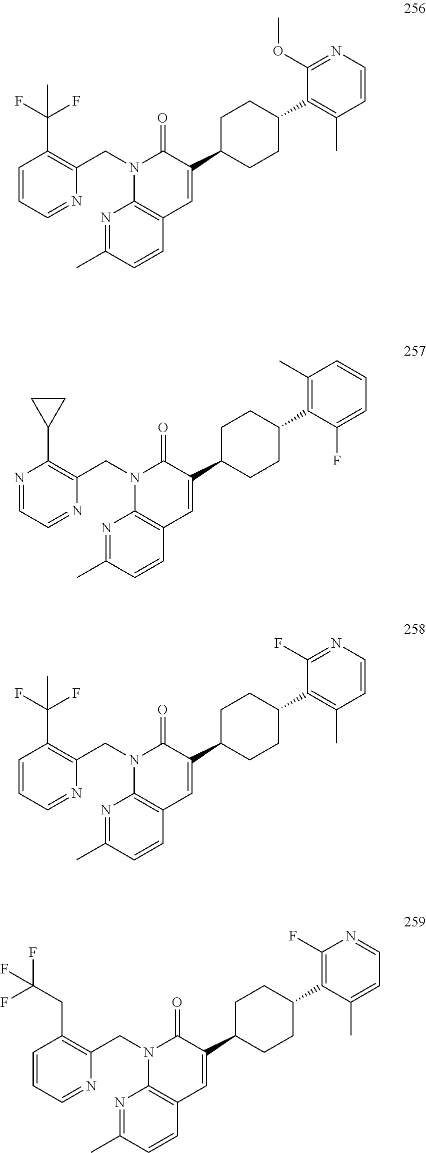 [SMILES: COc1nccc(C)c1[C@H]1CC[C@H](c2cc3ccc(C)nc3n(Cc3ncccc3C(C)(F)F)c2=O)CC1 ; Cc1ccc2cc([C@H]3CC[C@H](c4c(C)cccc4F)CC3)c(=O)n(Cc3nccnc3C3CC3)c2n1 ; Cc1ccc2cc([C@H]3CC[C@H](c4c(C)ccnc4F)CC3)c(=O)n(Cc3ncccc3C(C)(F)F)c2n1 ; Cc1ccc2cc([C@H]3CC[C@H](c4c(C)ccnc4F)CC3)c(=O)n(Cc3ncccc3CC(F)(F)F)c2n1]

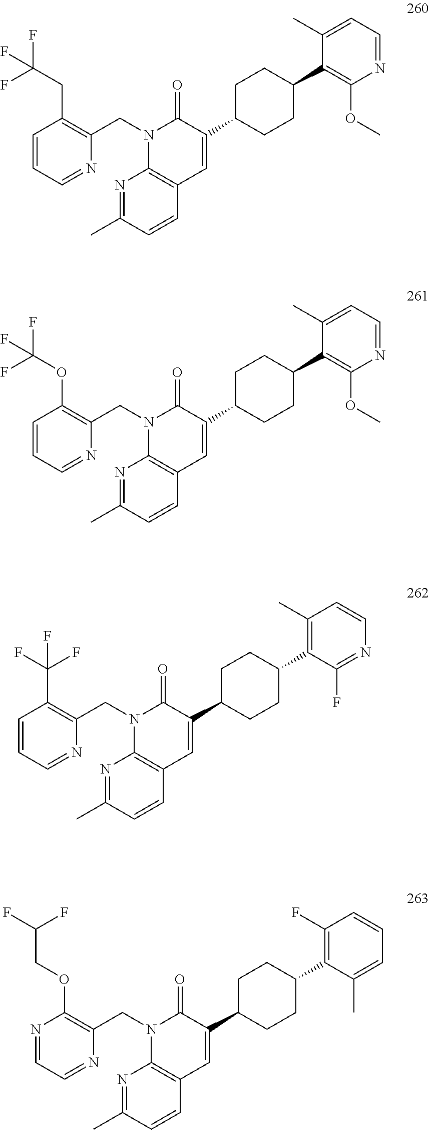 [SMILES: COc1nccc(C)c1[C@H]1CC[C@H](c2cc3ccc(C)nc3n(Cc3ncccc3CC(F)(F)F)c2=O)CC1 ; COc1nccc(C)c1[C@H]1CC[C@H](c2cc3ccc(C)nc3n(Cc3ncccc3OC(F)(F)F)c2=O)CC1 ; Cc1ccc2cc([C@H]3CC[C@H](c4c(C)ccnc4F)CC3)c(=O)n(Cc3ncccc3C(F)(F)F)c2n1 ; Cc1ccc2cc([C@H]3CC[C@H](c4c(C)cccc4F)CC3)c(=O)n(Cc3nccnc3OCC(F)F)c2n1]

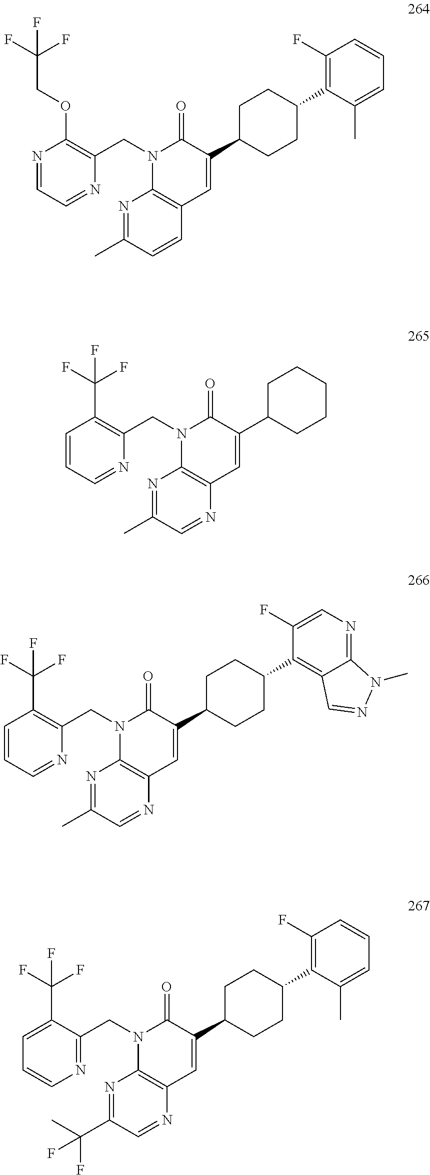 [SMILES: Cc1ccc2cc([C@H]3CC[C@H](c4c(C)cccc4F)CC3)c(=O)n(Cc3nccnc3OCC(F)(F)F)c2n1 ; Cc1cnc2cc(C3CCCCC3)c(=O)n(Cc3ncccc3C(F)(F)F)c2n1 ; Cc1cnc2cc([C@H]3CC[C@H](c4c(F)cnc5c4cnn5C)CC3)c(=O)n(Cc3ncccc3C(F)(F)F)c2n1 ; Cc1cccc(F)c1[C@H]1CC[C@H](c2cc3ncc(C(C)(F)F)nc3n(Cc3ncccc3C(F)(F)F)c2=O)CC1]

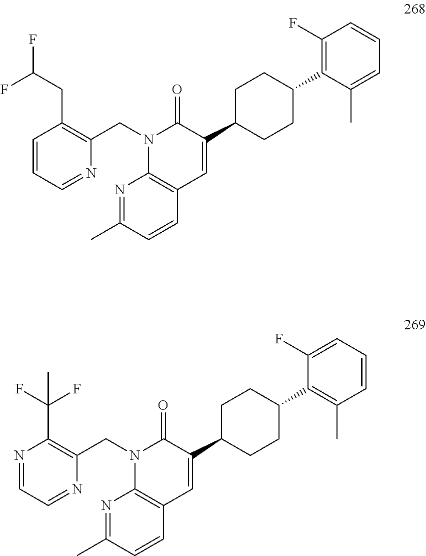 [SMILES: Cc1ccc2cc([C@H]3CC[C@H](c4c(C)cccc4F)CC3)c(=O)n(Cc3ncccc3CC(F)F)c2n1 ; Cc1ccc2cc([C@H]3CC[C@H](c4c(C)cccc4F)CC3)c(=O)n(Cc3nccnc3C(C)(F)F)c2n1]

In certain embodiments, the provided compounds (e.g., compounds of Formula (I)), antagonize C5aR1 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

In certain embodiments, the provided compounds (e.g., compounds of Formula (I)), inhibit C5aR1 with an IC₅₀ of less than 100,000 nM, less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

### Pharmaceutical Compositions, Kits, and Administration

The present disclosure provides pharmaceutical compositions comprising a disclosed compound (e.g., a compound of Formula (I)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of any of the formulae herein (e.g., Formula (I) or a pharmaceutically acceptable salt, co-crystal, tautomer, stereoisomer, solvate, hydrate, polymorph, isotopically enriched derivative, or prodrug thereof) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease or disorder in a subject in need thereof. In certain embodiments, the effective amount is an amount effective for treating cancer, an infectious disease, an autoimmune disease or disorder, an inflammatory disease or disorder, a cardiovascular disease or disorder, a cerebrovascular disease or disorder, a vasculitis disease or disorder, or a neurodegenerative disease or disorder. In certain embodiments, the effective amount is an amount effective for treating an autoimmune, inflammatory, or neurological disease or disorder. In certain embodiments, the effective amount is an amount effective for treating a neurological disease or disorder in a subject in need thereof. In certain embodiments, the effective amount is an amount effective for preventing cancer, an infectious disease, an autoimmune disease or disorder, an inflammatory disease or disorder, a cardiovascular disease or disorder, a cerebrovascular disease or disorder, a vasculitis disease or disorder, or a neurodegenerative disease or disorder. In certain embodiments, the effective amount is an amount effective for preventing an autoimmune, inflammatory, or neurological disease or disorder. In certain embodiments, the effective amount is an amount effective for preventing a neurological disease or disorder in a subject in need thereof.

In certain embodiments, the effective amount is an amount effective for reducing the risk of developing a disease (e.g., neurological disease or disorder) in a subject in need thereof.

In certain embodiments, the effective amount is an amount effective for antagonizing C5aR1 in a subject, tissue, biological sample, or cell.

In certain embodiments, the subject being treated or administered a compound described herein is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for decreasing the activity of C5aR1 by at least about 10%, at least about 20%, at least about 30%, at least about 40%, at least about 50%, at least about 60%, at least about 70%, at least about 80%, at least about 90%, at least about 95%, at least about 100%, at least about 150%, at least about 200%, at least about 250%, at least about 300%, at least about 400%, at least about 500%, or at least about 1000%. In certain embodiments, the effective amount is an amount effective for decreasing the activity of C5aR1 by a range between a percentage described in this paragraph and another percentage described in this paragraph, inclusive.

The present disclosure provides pharmaceutical compositions comprising a compound that interacts with (e.g., activates) C5aR1 for use in treating a C5aR1-related disease or disorder in a subject in need thereof. The present disclosure provides pharmaceutical compositions comprising a compound that interacts with (e.g., antagonizes) C5aR1 for use in treating a disease or disorder associated with aberrant activity of C5aR1 in a subject in need thereof. The present disclosure provides pharmaceutical compositions comprising a compound that interacts with (e.g., antagonizes) C5aR1 for use in treating a disease or disorder associated with mutated C5aR1 in a subject in need thereof.

In certain embodiments, the composition is for use in treating a disease or disorder. In certain embodiments, the composition is for use in treating an autoimmune, inflammatory, or neurological disease or disorder. In certain embodiments, the composition is for use in treating a neurological disease or disorder.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., neurological disease or disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the compound or pharmaceutical composition is a solid. In certain embodiments, the compound or pharmaceutical composition is a powder. In certain embodiments, the compound or pharmaceutical composition can be dissolved in a liquid to make a solution. In certain embodiments, the compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

After formulation with an appropriate pharmaceutically acceptable excipient in a desired dosage, the pharmaceutical compositions of the present disclosure can be administered to humans and other animals orally, parenterally, intracisternally, intraperitoneally, topically, bucally, or the like, depending on the disease or condition being treated.

In certain embodiments, a pharmaceutical composition comprising a compound of any of the formulae herein (e.g., Formula (I) or a pharmaceutically acceptable salt, co-crystal, tautomer, stereoisomer, solvate, hydrate, polymorph, isotopically enriched derivative, or prodrug thereof) is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Pharmaceutical compositions described herein can be prepared by any method known in the art of pharmacology. In general, such preparatory methods include the steps of bringing the composition comprising a compound of Formula (I) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g. polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g. Cremophor™), polyoxyethylene ethers, (e.g. polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazelnut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the disclosure are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this disclosure. Additionally, the present disclosure contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., liquids and gels), an anhydrous oil or silicone-based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., neurological disease or disorder) in a subject in need thereof. In certain embodiments, the kits are useful for preventing a disease (e.g., neurological disease or disorder) in a subject in need thereof. In certain embodiments, the kits are useful for reducing the risk of developing a disease (e.g., neurological disease or disorder) in a subject in need thereof. In certain embodiments, the kits are useful for decreasing the activity of C5aR1 in a subject or cell. In certain embodiments, the kits are useful for antagonizing C5aR1 in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., neurological disease or disorder) in a subject in need thereof. In certain embodiments, the kits and instructions provide for preventing a disease (e.g., neurological disease or disorder) in a subject in need thereof. In certain embodiments, the kits and instructions provide for reducing the risk of developing a disease (e.g., neurological disease or disorder) in a subject in need thereof. In certain embodiments, the kits and instructions provide for decreasing the activity of C5aR1 in a subject or cell. In certain embodiments, the kits and instructions provide for antagonizing C5aR1 in a subject or cell. A kit described herein may include one or more additional pharmaceutical agents described herein as a separate composition.

### Methods of Treatment

The present disclosure provides methods for treating a disease or disorder in a subject in need thereof. In certain embodiments, the present disclosure provides methods for treating a disease or disorder associated with C5aR1 (e.g., C5aR1 activity).

In certain embodiments, the disease or disorder is any of the following:

Autoimmune disorders (e.g., Rheumatoid arthritis, systemic lupus erythematosus, Guillain-Barre syndrome, pancreatitis, lupus nephritis, lupus glomerulonephritis, psoriasis, Crohn's disease, vasculitis, irritable bowel syndrome, dermatomyositis, multiple sclerosis, bronchial asthma, pemphigus, pemphigoid, scleroderma, myasthenia gravis, autoimmune hemolytic and thrombocytopenic states, aHUS (Atypical Hemolytic Uremic Syndrome), Goodpasture's syndrome (and associated glomerulonephritis and pulmonary hemorrhage), immunovasculitis, tissue graft rejection, hyperacute rejection of transplanted organs).

Inflammatory disorders and related conditions (e.g., Neutropenia, sepsis, septic shock, Alzheimer's disease, multiple sclerosis, stroke, inflammatory bowel disease (IBD), age-related macular degeneration (AMD, both wet and dry forms), inflammation associated with severe burns, lung injury, and ischemia-reperfusion injury, osteoarthritis, as well as acute (adult) respiratory distress syndrome (ARDS), chronic pulmonary obstructive disorder (COPD), systemic inflammatory response syndrome (SIRS), atopic dermatitis, psoriasis, chronic urticaria and multiple organ dysfunction syndrome (MODS). Also included are pathologic sequellae associated with insulin-dependent diabetes mellitus (including diabetic retinopathy), lupus nephropathy, Heyman nephritis, membranous nephritis and other forms of glomerulonephritis, IGA nephropathy, contact sensitivity responses, and inflammation resulting from contact of blood with artificial surfaces that can cause complement activation, as occurs, for example, during extracorporeal circulation of blood (e.g., during hemodialysis or via a heart-lung machine, for example, in association with vascular surgery such as coronary artery bypass grafting or heart valve replacement), or in association with contact with other artificial vessel or container surfaces (e.g., ventricular assist devices, artificial heart machines, transfusion tubing, blood storage bags, plasmapheresis, plateletpheresis, and the like), diseases related to ischemia/reperfusion injury, such as those resulting from transplants, including solid organ transplant, and syndromes such as ischemic reperfusion injury, ischemic colitis and cardiac ischemia. Compounds of the instant disclosure may also be useful in the treatment of age-related macular degeneration (Hageman et al, P.N.A.S. 102: 7227-7232, 2005).

Cardiovascular and cerebrovascular disorders (e.g., myocardial infarction, coronary thrombosis, vascular occlusion, post-surgical vascular reocclusion, atherosclerosis, traumatic central nervous system injury, and ischemic heart disease. In one embodiment, an effective amount of a compound of the disclosure may be administered to a patient at risk for myocardial infarction or thrombosis (i.e., a patient who has one or more recognized risk factor for myocardial infarction or thrombosis, such as, but not limited to, obesity, smoking, high blood pressure, hypercholesterolemia, previous or genetic history of myocardial infarction or thrombosis) in order reduce the risk of myocardial infarction or thrombosis).

Diseases of vasculitis: vasculitic diseases are characterized by inflammation of the vessels. Infiltration of leukocytes leads to destruction of the vessel walls, and the complement pathway is believed to play a major role in initiating leukocyte migration as well as the resultant damage manifested at the site of inflammation (Vasculitis, Second Edition, Edited by Ball and Bridges, Oxford University Press, pp 47-53, 2008). The compounds provided in the present disclosure can be used to treat ANCA vasculitis (anti-neutrophil cytoplasmic autoantibody vasculitis). The compounds provided in the present disclosure can be used to treat leukoclastic vasculitis, urticarial vasculitis, Wegener's granulomatosis, microscopic polyangiitis, Churg-Strauss syndrome, Henoch-Schonlein purpura, polyateritis nodosa, Rapidly Progressive Glomerulonephritis (RPGN), cryoglobulinaemia, giant cell arteritis (GCA), Behcet's disease and Takayasu's arteritis (TAK).

HIV infection and AIDS (e.g., C5a receptor modulators provided herein may be used to inhibit HIV infection, delay AIDS progression or decrease the severity of symptoms or HIV infection and AIDS).

Neurodegenerative disorders and related diseases (e.g., Alzheimer's disease, multiple sclerosis, and cognitive function decline associated with cardiopulmonary bypass surgery and related procedures).

Cancer and precancerous conditions (e.g., melanomas, lung cancer, lymphomas, sarcomas, carcinomas, and mixed tumors. Exemplary conditions that may be treated according to the present disclosure include fibrosarcomas, liposarcomas, chondrosarcomas, osteogenic sarcomas, angiosarcomas, lymphangiosarcomas, synoviomas, mesotheliomas, meningiomas, leukemias, lymphomas, leiomyosarcomas, rhabdomyosarcomas, squamous cell carcinomas, basal cell carcinomas, adenocarcinomas, papillary carcinomas, cystadenocarcinomas, bronchogenic carcinomas, melanomas, renal cell carcinomas, hepatocellular carcinomas, transitional cell carcinomas, choriocarcinomas, seminomas, embryonal carcinomas, Wilm's tumors, pleomorphic adenomas, liver cell papillomas, renal tubular adenomas, cystadenomas, papillomas, adenomas, leiomyomas, rhabdomyomas, hemangiomas, lymphangiomas, osteomas, chondromas, lipomas and fibromas. In some embodiments, the disease or disorder is selected from the group consisting of glioblastoma, esophagus tumor, nasopharyngeal carcinoma, uveal melanoma, lymphoma, lymphocytic lymphoma, primary CNS lymphoma, T-cell lymphoma, diffuse large B-cell lymphoma, primary mediastinal large B-cell lymphoma, prostate cancer, castration-resistant prostate cancer, chronic myelocytic leukemia, Kaposi's sarcoma fibrosarcoma, liposarcoma, chondrosarcoma, osteogenic sarcoma, angiosarcoma, lymphangiosarcoma, synovioma, meningioma, leiomyosarcoma, rhabdomyosarcoma, sarcoma of soft tissue, sarcoma, sepsis, biliary tumor, basal cell carcinoma, thymus neoplasm, cancer of the thyroid gland, cancer of the parathyroid gland, uterine cancer, cancer of the adrenal gland, liver infection, Merkel cell carcinoma, nerve tumor, follicle center lymphoma, colon cancer, Hodgkin's disease, non-Hodgkin's lymphoma, leukemia, chronic or acute leukemias including acute myeloid leukemia, chronic myeloid leukemia, acute lymphoblastic leukemia, chronic lymphocytic leukemia, multiple myeloma, ovary tumor, myelodysplastic syndrome, cutaneous or intraocular malignant melanoma, renal cell carcinoma, small-cell lung cancer, lung cancer, mesothelioma, breast cancer, squamous non-small cell lung cancer (SCLC), non-squamous NSCLC, colorectal cancer, ovarian cancer, gastric cancer, hepatocellular carcinoma, pancreatic carcinoma, pancreatic cancer, Pancreatic ductal adenocarcinoma, squamous cell carcinoma of the head and neck, cancer of the head or neck, gastrointestinal tract, stomach cancer, bone cancer, skin cancer, rectal cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the urethra, cancer of the penis, cancer of the bladder, cancer of the kidney, cancer of the ureter, carcinoma of the renal pelvis, neoplasm of the central nervous system (CNS), tumor angiogenesis, spinal axis tumor, brain stem glioma, pituitary adenoma, epidermoid cancer, asbestosis, and carcinoma).

In one embodiment, an effective amount of a compound of the disclosure may be administered to a patient at risk for myocardial infarction or thrombosis (i.e., a patient who has one or more recognized risk factor for myocardial infarction or thrombosis, such as, but not limited to, obesity, smoking, high blood pressure, hypercholesterolemia, previous or genetic history of myocardial infarction or thrombosis) in order reduce the risk of myocardial infarction or thrombosis.

In another embodiment, the compounds of the present disclosure are useful in the treatment of cisplatin induced nephrotoxicity. In this embodiment, compound treatment can alleviate the nephrotoxicity induced by cisplatin chemotherapy of malignancies (Hao Pan et al, Am J Physiol Renal Physiol, 296, F496-504, 2009).

In one embodiment, the compounds of the disclosure can be used for the treatment of diseases selected from the group consisting of sepsis (and associated disorders), COPD, rheumatoid arthritis, lupus nephritis and multiple sclerosis.

Provided herein is a method of treating a human suffering from or susceptible to a disease or disorder involving pathologic activation of C5a receptors, comprising administering a therapeutically effective amount of a compound of the disclosure or a pharmaceutical composition thereof.

Provided herein is a method of inhibiting C5a receptor-mediated cellular chemotaxis comprising contacting mammalian white blood cells with a C5a receptor modulatory amount of an active metabolite of a compound of the disclosure.

In some embodiments, the disease or disorder is an inflammatory disease or disorder, a cardiovascular or cerebrovascular disorder, an autoimmune disease, or an oncologic disease or disorder.

In some embodiments, the disease or disorder is selected from the group consisting of neutropenia, neutrophilia, C3-glomerulopathy, C3-glomerulonephritis, dense deposit disease, membranoproliferative glomerulonephritis, Kawasaki disease, sepsis, septic shock, Hemolytic uremic syndrome, atypical hemolytic uremic syndrome (aHUS), Alzheimer's disease, multiple sclerosis, stroke, inflammatory bowel disease, chronic obstructive pulmonary disorder, inflammation associated with burns, lung injury, osteoarthritis, atopic dermatitis, chronic urticaria, ischemia-reperfusion injury, acute respiratory distress syndrome, systemic inflammatory response syndrome, multiple organ dysfunction syndrome, Uveitis, tissue graft rejection, hyperacute rejection of transplanted organs, myocardial infarction, coronary thrombosis, vascular occlusion, post-surgical vascular reocclusion, artherosclerosis, polypoidal choroidal vasculopathy, traumatic central nervous system injury, ischemic heart disease, rheumatoid arthritis, systemic lupus erythematosus, Guillain-Barre syndrome, pancreatitis, lupus nephritis, lupus glomerulonephritis, psoriasis, Crohn's disease, vasculitis, ANCA vasculitis, irritable bowel syndrome, dermatomyositis, multiple sclerosis, bronchial asthma, pemphigus, pemphigoid, scleroderma, myasthenia gravis, autoimmune hemolytic and thrombocytopenic states, Goodpasture's syndrome, immuno vasculitis, Graft versus host disease, Paroxysmal nocturnal hemoglobinuria, Sjoegrens syndrome, insulin-dependent diabetes, mellitus, lupus nephropathy, Heyman nephritis, membranous nephritis, glomerulonephritis, IGA nephropathy, Membranoproliferative glomerulonephritis, Antiphospholipid syndrome, Age related macular degeneration; Dry age related macular degeneration, Wet age related macular degeneration, Motor neurone disease, contact sensitivity responses, and inflammation resulting from contact of blood with artificial surfaces.

In some embodiments, the disease or disorder is selected from the group consisting of neutropenia, neutrophilia, C3-glomerulopathy, C3-glomerulonephritis, dense deposit disease, membranoproliferative glomerulonephritis, Kawasaki disease, Hemolytic uremic syndrome, atypical hemolytic uremic syndrome (aHUS), tissue graft rejection, hyperacute rejection of transplanted organs, rheumatoid arthritis, systemic lupus erythematosus, lupus nephritis, lupus glomerulonephritis, vasculitis, ANCA vasculitis, autoimmune hemolytic and thrombocytopenic states, immuno vasculitis, Graft versus host disease, lupus nephropathy, Heyman nephritis, membranous nephritis, glomerulonephritis, IGA nephropathy, Membranoproliferative and glomerulonephritis.

In some embodiments, the disease or disorder is selected from the group consisting of melanoma, lung cancer, lymphoma, sarcoma, carcinoma, fibrosarcoma, liposarcoma, chondrosarcoma, osteogenic sarcoma, angiosarcoma, lymphangiosarcoma, synovioma, mesothelioma, meningioma, leukemia, lymphoma, leiomyosarcoma, rhabdomyosarcoma, squamous cell carcinoma, basal cell carcinoma, adenocarcinoma, papillary carcinoma, cystadenocarcinoma, bronchogenic carcinoma, renal cell carcinoma, hepatocellular carcinoma, transitional cell carcinoma, choriocarcinoma, seminoma, embryonal carcinoma, Wilm's tumor, pleomorphic adenoma, liver cell papilloma, renal tubular adenoma, cystadenoma, papilloma, adenoma, leiomyoma, rhabdomyoma, hemangioma, lymphangioma, osteoma, chondroma, lipoma and fibroma. In some embodiments, the disease or disorder is selected from the group consisting of glioblastoma, esophagus tumor, nasopharyngeal carcinoma, uveal melanoma, lymphoma, lymphocytic lymphoma, primary CNS lymphoma, T-cell lymphoma, diffuse large B-cell lymphoma, primary mediastinal large B-cell lymphoma, prostate cancer, castration-resistant prostate cancer, chronic myelocytic leukemia, Kaposi's sarcoma fibrosarcoma, liposarcoma, chondrosarcoma, osteogenic sarcoma, angiosarcoma, lymphangiosarcoma, synovioma, meningioma, leiomyosarcoma, rhabdomyosarcoma, sarcoma of soft tissue, sarcoma, sepsis, biliary tumor, basal cell carcinoma, thymus neoplasm, cancer of the thyroid gland, cancer of the parathyroid gland, uterine cancer, cancer of the adrenal gland, liver infection, Merkel cell carcinoma, nerve tumor, follicle center lymphoma, colon cancer, Hodgkin's disease, non-Hodgkin's lymphoma, leukemia, chronic or acute leukemias including acute myeloid leukemia, chronic myeloid leukemia, acute lymphoblastic leukemia, chronic lymphocytic leukemia, multiple myeloma, ovary tumor, myelodysplastic syndrome, cutaneous or intraocular malignant melanoma, renal cell carcinoma, small-cell lung cancer, lung cancer, mesothelioma, breast cancer, squamous non-small cell lung cancer (SCLC), non-squamous NSCLC, colorectal cancer, ovarian cancer, gastric cancer, hepatocellular carcinoma, pancreatic carcinoma, pancreatic cancer, pancreatic ductal adenocarcinoma, squamous cell carcinoma of the head and neck, cancer of the head or neck, gastrointestinal tract, stomach cancer, bone cancer, skin cancer, rectal cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the urethra, cancer of the penis, cancer of the bladder, cancer of the kidney, cancer of the ureter, carcinoma of the renal pelvis, neoplasm of the central nervous system (CNS), tumor angiogenesis, spinal axis tumor, brain stem glioma, pituitary adenoma, epidermoid cancer, asbestosis, and carcinoma.

In certain embodiments, the autoimmune disease or disorder is acquired aplastic anemia, acute disseminated encephalomyelitis (ADEM), acute hemorrhagic leukoencephalitis (AHLE)/Hurst's disease, gammaglobulinemia, (primary), alopecia areata, ankylosing spondylitis (AS), anti-NMDA receptor encephalitis, antiphospholipid syndrome (APS), arteriosclerosis, autism spectrum disorders (ASD), autoimmune Addison's disease (AAD), autoimmune dysautonomia/Autoimmune autonomic ganglionopathy (AAG), autoimmune encephalitis, autoimmune gastritis, autoimmune hemolytic anemia (AIHA), autoimmune hepatitis (AIH), autoimmune hyperlipidemia, autoimmune hypophysitis/lymphocytic hypophysitis, autoimmune inner ear disease (AIED), autoimmune lymphoproliferative syndrome (ALPS), autoimmune myocarditis, autoimmune oophoritis, autoimmune orchitis, autoimmune pancreatitis (AIP)/Immunoglobulin G4-Related Disease (IgG4-RD), autoimmune polyglandular syndromes (Types I, II, & III), autoimmune progesterone dermatitis, autoimmune sudden sensorineural hearing loss (SNHL), balo disease, Behcet's disease, birdshot chorioretinopathy/birdshot uveitis, bullous pemphigoid, castleman disease, celiac disease, chagas disease, chronic fatigue syndrome (CFS)/myalgic encephalomyelitis (ME), chronic inflammatory demyelinating polyneuropathy (CIDP), chronic Lyme disease/post-treatment Lyme disease syndrome (PTLDS), chronic urticaria (CU), churg-Strauss syndrome/eosinophilic granulomatosis with polyangiitis (EGPA), cicatricial pemphigoid, Cogan's syndrome, cold agglutinin disease, CREST syndrome/limited cutaneous systemic sclerosis, Crohn's disease (CD), cronkhite-Canada syndrome (CSS), cryptogenic organizing pneumonia (COP), dermatitis herpetiformis, dermatomyositis, type 1 diabetes, discoid lupus, Dressler's syndrome/postmyocardial infarction/postpericardiotomy syndrome, endometriosis, eosinophilic fasciitis, erythema nodosum, essential mixed cryoglobulinemia, Evans syndrome, fibrosing alveolitis, giant cell arteritis/temporal arteritis/Horton's disease, glomerulonephritis, Goodpasture's syndrome/anti-GBM/anti-TBM disease, granulomatosis with polyangiitis (GPA)/Wegener's granulomatosis, Graves' disease, Guillain-Barre syndrome (GBS), Hashimoto's thyroiditis/chronic lymphocytic thyroiditis/autoimmune thyroiditis, Henoch-Schonlein purpura/IgA vasculitis, herpes gestationis/pemphigoid gestationis, hypogammaglobulinemia, IgA nephropathy/Berger's disease, immune thrombocytopenia (ITP)/autoimmune thrombocytopenic purpura, interstitial cystitis juvenile idiopathic arthritis, Kawasaki disease, Lambert-Eaton myasthenic syndrome (LEMS), leukocytoclastic vasculitis, lichen planus, lichen sclerosus, ligneous conjunctivitis, linear IgA disease (LAD)/linear IgA bullous dermatosis (LABD), lupus nephritis, Meniere's disease, microscopic polyangiitis (MPA), mixed connective tissue disease (MCTD), Mooren's ulcer, Mucha-Habermann disease, multiple sclerosis (MS), myasthenia gravis (MG), neuromyelitis optica (NMO)/Devic's disease, narcolepsy, non-length-dependent small fiber sensory neuropathy (SFSN), ocular cicatricial pemphigoid, opsoclonus-myoclonus syndrome (OMS), palindromic rheumatism, palmoplantar pustulosis (type of psoriasis), paraneoplastic cerebellar degeneration, paraneoplastic pemphigus, paroxysmal nocturnal hemoglobinuria (PNH), peripheral uveitis/pars planitis, parsonage turner syndrome, pemphigus foliaceus, pemphigus vulgaris, pernicious anemia, POEMS syndrome, polyarteritis nodosa, polymyalgia rheumatica, polymyositis, postural orthostatic tachychardia syndrome (POTS), primary biliary cholangitis (PBC)/primary biliary cirrhosis, primary sclerosing cholangitis (PSC), psoriasis, psoriatic arthritis, pulmonary fibrosis, idiopathic (IPF), pure red cell aplasia, pyoderma gangrenosum, Raynaud's phenomenon, reactive arthritis/Reiter's syndrome, reflex sympathetic dystrophy syndrome, relapsing polychondritis, leg syndrome/Willis-Ekbom disease, rheumatic fever, rheumatoid arthritis, sarcoidosis, Schmidt syndrome/autoimmune poly endocrine syndrome type II, systemic sclerosis, scleritis, scleroderma, serpiginous choroidopathy, Sjogren's syndrome, stiff person syndrome (SPS), systemic lupus erythematosus (SLE), subacute bacterial endocarditis (SBE), Sydenham's chorea, sympathetic ophthalmia, Takayasu's arteritis (vasculitis), testicular autoimmunity (vasculitis/orchitis), Tolosa-Hunt syndrome, transverse myelitis (TM), tubulointerstitial nephritis uveitis syndrome (TINU), ulcerative colitis (UC), undifferentiated connective tissue disease (UCTD), uveitis (anterior), uveitis (intermediate), uveitis (posterior), vasculitis, vitiligo, or Vogt-Koyanagi-Harada syndrome (VKH). In one embodiment, the autoimmune trigger is lupus nephritis. In another embodiment, the autoimmune trigger is systemic sclerosis.

In certain embodiments, the disease or disorder is age-related macular degeneration, Alzheimer's disease, amyotrophic lateral sclerosis, anaphylaxis, anthrax poisoning, anti-neutrophil cytoplasmic antibody-associated vasculitis, antiphospholipid syndrome, asthma, atherosclerosis, atopic dermatitis, atypical hemolytic uremic syndrome, autism, autoantibody-mediated disease, Berger disease, brain ischaemia, bronchiectasis, C3 glomerulopathy, cancer, Carnevale syndrome, CHAPLEE syndrome, chron disease, chronic obstructive pulmonary disease, cold agglutin disease, cutaneous lupus erythematosus, dementia, dermatomyositis, diabetic angiopathy, Ehlers-Danlos, epilepsy, frontotemporal dementia, generalized myasthenia gravis, geographic atrophy, glaucoma, glomerulonephritis, Guillain-Barre syndrome, hematopoetic stem cell transplant, complement mediated rejection, hereditary angioedema, hidradenitis supprativa, Huntington's disease, IgA nephropathy, immune thrombocytopenia, lupus nephritis, Malpuech syndrome, membranous glomerulonephritis, Michels syndrome, microscopic polyangiitis, Mingarelli syndrome, mood disorders, multifocal motor neuropathy, multiple sclerosis, myocardial infarction, neonatal HIE, neuromyelitis, optica spectrum disorder, neurotrauma, osteoarthritis, palmoplantar pustulosis, Parkinson's disorder, paroxysmal nocturnal haemoglobinuria, pemphigus, polytrauma, preeclampsia, primary membranous nephropathy, psoriasis, pyoderma gangraenosum, rare neutrophilic dermatoses, rheumatoid arthritis, schizophrenia, sepsis, severe thrombocytopenia, Shiga toxin associated hemolytic uremic syndrome, spinal cord injury, Stargardt disease, stroke, Sweet syndrome, systemic lupus erythematosus, systemic sclerosis, thrombotic microangiopathies, transplant rejection, Traumatic brain injury, ulcerative colitis, or vasculitis.

In certain embodiments, the application provides a method of treating cancer, an infectious disease, an autoimmune disease or disorder, an inflammatory disease or disorder, a cardiovascular disease or disorder, a cerebrovascular disease or disorder, a vasculitis disease or disorder, or a neurodegenerative disease or disorder.

In certain embodiments, the application provides a method of treating an autoimmune, inflammatory, or neurological disease or disorder. In certain embodiments, the application provides a method of treating a neurological disease or disorder.

The present disclosure provides a method of antagonizing C5aR1. The present disclosure provides a method of decreasing the activity of C5aR1. In certain embodiments, the application provides a method of antagonizing C5aR1 (e.g., decreasing the activity of C5aR1) in vitro. In certain embodiments, the application provides a method of antagonizing C5aR1 (e.g., decreasing the activity of C5aR1) in vivo. In certain embodiments, the application provides a method of antagonizing C5aR1 in a cell. In certain embodiments, the application provides a method of antagonizing C5aR1 in a human cell.

In certain embodiments, the methods comprise administering to a subject in need thereof (e.g., a subject with a neurological disease or disorder) a compound that interacts with C5aR1, for example, a compound that is a modulator of C5aR1 (e.g., an antagonist of C5aR1), a binder of C5aR1, or a compound that modifies C5aR1. In certain embodiments, the methods comprise administering a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof. In some embodiments, the method comprises administering a pharmaceutical composition comprising a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof, to a subject in need thereof.

Another object of the present disclosure is the use of a compound as described herein (e.g., of any formulae herein) in the manufacture of a medicament for use in the treatment of a disorder or disease described herein. Another object of the present disclosure is the use of a compound as described herein (e.g., of any formulae herein) for use in the treatment of a disorder or disease described herein.

### EXAMPLES

In order that the disclosure described herein may be more fully understood, the following examples are set forth. The examples described in this application are offered to illustrate the compounds, pharmaceutical compositions, and methods provided herein and are not to be construed in any way as limiting their scope.

### Synthetic Methods

Compounds of Formula (I) were prepared following the synthetic schemes and procedures described in detail below. The examples described in this application are offered to illustrate the compounds, pharmaceutical compositions, and methods provided herein and are not to be construed in any way as limiting their scope. Compounds of the disclosure that are not explicitly described in the following procedures may be prepared by analogous methods. Those having ordinary skill in the art would understand how to make such compounds from the disclosure provided herein and by means known in the art of organic synthesis. For example, those such as described in R. Larock, Comprehensive Organic Transformations, VCH Publishers (1989); T. W. Greene and P.G.M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, if necessary minimizing competing by products, are known in the art. Embodiments of this disclosure include methods of synthesizing compounds delineated herein using any of the compounds, reactants, and/or processes delineated herein.

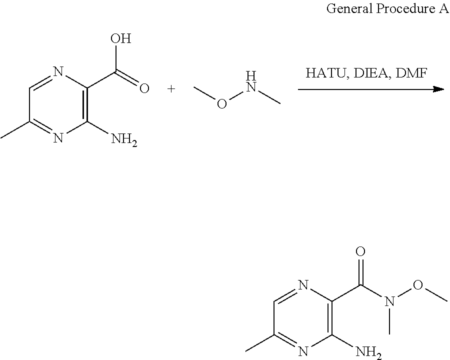

3-Amino-N-methoxy-N,5-dimethylpyrazine-2-carboxamide: To a stirred mixture of 3-amino-5-methylpyrazine-2-carboxylic acid (300 mg, 1.95 mmol, 1 equiv) and N,O-dimethylhydroxylamine (179 mg, 2.93 mmol, 1.5 equiv) in DMF (5.00 mL) were added HATU (1120 mg, 2.93 mmol, 1.5 equiv) and DIEA (1260 mg, 9.79 mmol, 3 equiv). The resulting mixture was stirred for 2 h at room temperature under nitrogen atmosphere. The resulting mixture was diluted with water (20 mL). The resulting mixture was extracted with EtOAc (3×20 mL). The combined organic layers were washed with brine, dried over anhydrous Na₂SO₄. After filtration, the filtrate was concentrated under reduced pressure. The residue was purified by silica gel column chromatography, eluted with EtOAc/PE=10/1 to afford 3-amino-N-methoxy-N,5-dimethylpyrazine-2-carboxamide (320 mg, 83%) as an off-white solid.

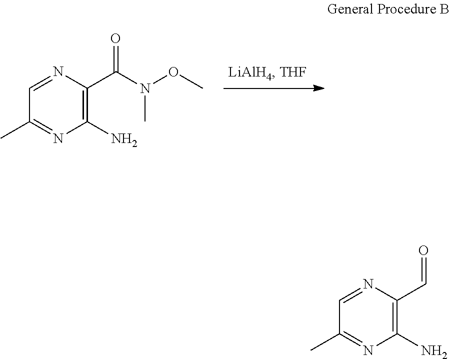

3-Amino-5-methylpyrazine-2-carbaldehyde: To a stirred mixture of 3-amino-N-methoxy-N,5-dimethylpyrazine-2-carboxamide (320 mg, 1.63 mmol, 1 equiv) in THF (2 mL) were added LiAlH₄ (1M in THF, 1.95 mL, 1.95 mmol, 1.2 equiv) dropwise at 0° C. under nitrogen atmosphere. The resulting mixture was stirred for 1 h at 0° C. The desired product was detected by LCMS. The reaction was quenched by the addition of Na₂SO₄·10H₂O (147 mg, 2 equiv) at 0° C. The resulting mixture was filtered, the filter cake was washed with EtOAc (3×10 mL). The filtrate was concentrated under reduced pressure. This resulted in 3-amino-5-methylpyrazine-2-carbaldehyde (200 mg, 89%) as a yellow solid.

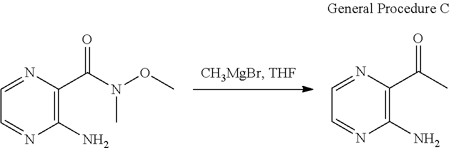

1-(3-Aminopyrazin-2-yl)ethan-1-one: To a stirred solution of 3-amino-N-methoxy-N-methylpyrazine-2-carboxamide (500 mg, 2.74 mmol, 1 equiv) in THF (10 mL) was added MeMgBr (3M in THF, 1.37 mL, 4.11 mmol, 1.5 equiv) dropwise at 0° C. under argon atmosphere. The resulting mixture was stirred for 1 h at 0° C. The reaction was quenched by the addition of sat. NH₄Cl (aq.) (10 mL) at 0° C. The resulting mixture was extracted with EtOAc (3×10 mL). The combined organic layers were washed with brine (20 mL), dried over anhydrous Na₂SO₄. After filtration, the filtrate was concentrated under reduced pressure. This resulted in 1-(3-aminopyrazin-2-yl)ethan-1-one (400 mg, crude) as an off-white solid.

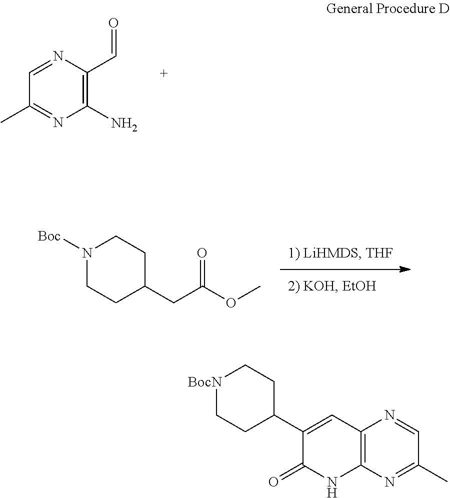

tert-Butyl 4-(3-methyl-6-oxo-5,6-dihydropyrido[2,3-b]pyrazin-7-yl)piperidine-1-carboxylate: To a stirred mixture of tert-butyl 4-(2-methoxy-2-oxoethyl)piperidine-1-carboxylate (450 mg, 1.75 mmol, 1.2 equiv) in THF (10 mL) was added LiHMDS (1M in THF, 8.75 mL, 8.75 mmol, 6 equiv) dropwise at −78° C. under argon atmosphere. The resulting mixture was stirred for 1 h at −78° C. To the above mixture was added 3-amino-5-methylpyrazine-2-carbaldehyde (200 mg, 1.46 mmol, 1 equiv) in THF (5 mL) dropwise over 5 min at −78° C. The resulting mixture was stirred for additional 5 h at −40° C. To the above mixture was added KOH (491 mg, 8.75 mmol, 6 equiv) and EtOH (10 mL) at room temperature. The resulting mixture was stirred for additional overnight at 80° C. The desired product was detected by LCMS. The resulting mixture was concentrated under reduced pressure. The resulting mixture was diluted with water (50 mL), extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with EtOAc/PE=10/1 to afford tert-butyl 4-(3-methyl-6-oxo-5,6-dihydropyrido[2,3-b]pyrazin-7-yl)piperidine-1-carboxylate (320 mg, 63%) as a light brown solid.

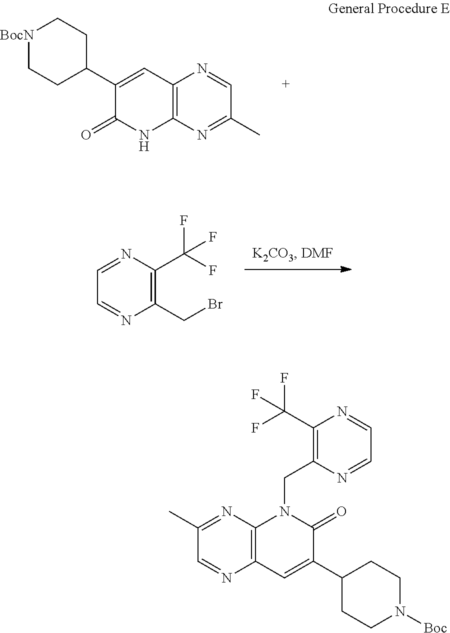 [SMILES: Cc1cnc2cc(C3CCN(C(=O)OC(C)(C)C)CC3)c(=O)[nH]c2n1 ; FC(F)(F)c1nccnc1CBr ; Cc1cnc2cc(C3CCN(C(=O)OC(C)(C)C)CC3)c(=O)n(Cc3nccnc3C(F)(F)F)c2n1]

tert-Butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate: To a solution of tert-butyl 4-(3-methyl-6-oxo-5,6-dihydropyrido[2,3-b]pyrazin-7-yl)piperidine-1-carboxylate (90 mg, 0.261 mmol, 1 equiv) and 2-(bromomethyl)-3-(trifluoromethyl)pyrazine (94.4 mg, 0.392 mmol, 1.5 equiv) in DMF (3 mL) was added K₂CO₃ (108 mg, 0.783 mmol, 3 equiv) under nitrogen atmosphere. The resulting mixture was stirred for overnight at room temperature. The resulting mixture was diluted with water (20 mL), extracted with EtOAc (3×20 mL). The combined organic layers were washed by brine (30 mL), dried over anhydrous Na₂SO₄. After filtration, the filtrate was concentrated under reduced pressure. The residue was purified by silica gel column chromatography, eluted with EtOAc/PE=2/1, to afford tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (70 mg, 53%) as a white solid.

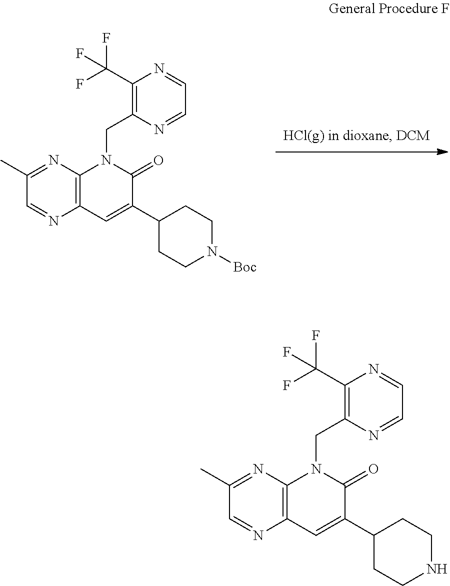 [SMILES: Cc1cnc2cc(C3CCN(C(=O)OC(C)(C)C)CC3)c(=O)n(Cc3nccnc3C(F)(F)F)c2n1 ; Cc1cnc2cc(C3CCNCC3)c(=O)n(Cc3nccnc3C(F)(F)F)c2n1]

3-Methyl-7-(piperidin-4-yl)-5-((3-(trifluoromethyl)pyrazin-2-yl)methyl)pyrido[2,3-b]pyrazin-6(5H)-one: A solution of tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (70 mg, 0.139 mmol, 1 equiv) and HCl (gas) in 1,4-dioxane (4M, 2.00 mL) in DCM (2.00 mL) was stirred for 1 h at room temperature. After removing the solvent, the mixture was basified to pH 8 with saturated NaHCO₃ (aq.). The resulting mixture was extracted with EtOAc (3×30 mL). The combined organic layers were washed with brine (30 mL), dried over anhydrous Na₂SO₄. After filtration, the filtrate was concentrated under reduced pressure. The crude product 3-methyl-7-(piperidin-4-yl)-5-((3-(trifluoromethyl)pyrazin-2-yl)methyl)pyrido[2,3-b]pyrazin-6(5H)-one (45 mg) was used for next step without further purification.

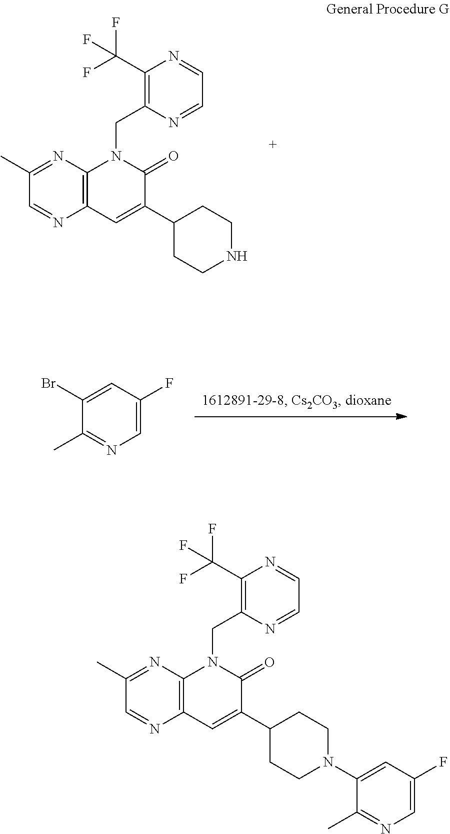

7-(1-(5-Fluoro-2-methylpyridin-3-yl)piperidin-4-yl)-3-methyl-5-((3-(trifluoromethyl)pyrazin-2-yl)methyl)pyrido[2,3-b]pyrazin-6(5H)-one: To the solution of 3-methyl-7-(piperidin-4-yl)-5-((3-(trifluoromethyl)pyrazin-2-yl)methyl)pyrido[2,3-b]pyrazin-6(5H)-one (45 mg, 0.111 mmol, 1 equiv) and 3-bromo-5-fluoro-2-methylpyridine (25.3 mg, 0.133 mmol, 1.2 equiv) in dioxane (3 mL) were added 1612891-29-8 (4.68 mg, 0.006 mmol, 0.05 equiv) and Cs₂CO₃ (108 mg, 0.333 mmol, 3 equiv) under argon atmosphere. The result mixture was stirred for overnight at 100° C. The resulting mixture was diluted with water (20 mL) and extracted with EtOAc (3×15 mL). The combined organic layers were washed with brine (30 mL), dried over anhydrous Na₂SO₄. After filtration, the filtrate was concentrated under reduced pressure. The residue was purified by silica gel column chromatography, eluted with EtOAc/PE=2/1, to afford 7-(1-(5-fluoro-2-methylpyridin-3-yl)piperidin-4-yl)-3-methyl-5-((3-(trifluoromethyl)pyrazin-2-yl)methyl)pyrido[2,3-b]pyrazin-6(5H)-one (19.3 mg, 33.4%) as a pink solid.

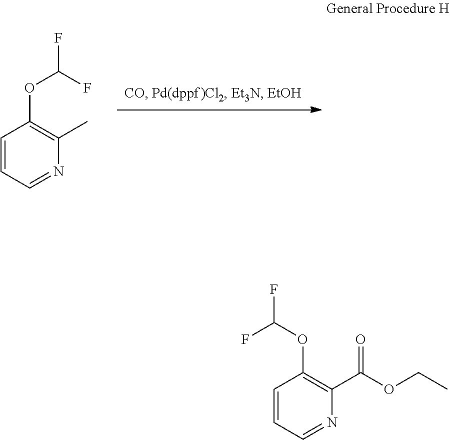

Ethyl 3-(difluoromethoxy)picolinate: To a solution of 2-bromo-3-(difluoromethoxy)pyridine (200 mg, 0.893 mmol, 1 equiv) and Pd(dppf)Cl₂ (65.3 mg, 0.089 mmol, 0.1 equiv) in EtOH (3 mL) was added Et₃N (271 mg, 2.67 mmol, 3 equiv) in a pressure tank. The mixture was purged with nitrogen for 5 min and then was pressurized to 50 atm with carbon monoxide at 100° C. for overnight. The reaction mixture was cooled to room temperature and filtered to remove insoluble solids. The desired product was detected by LCMS. After removing the solvent, the residue was purified by silica gel column chromatography, eluted with EtOAc/PE=1/1 to afford ethyl 3-(difluoromethoxy)picolinate (180 mg, 92.8%) as a light yellow oil.

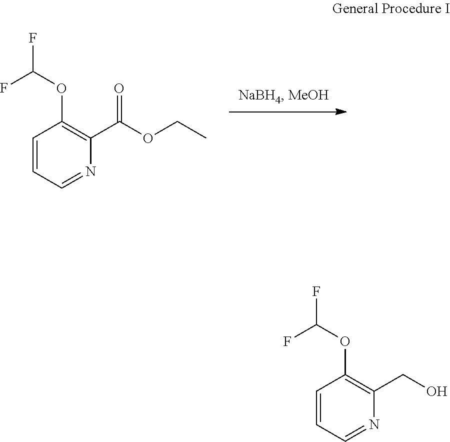

(3-(Difluoromethoxy)pyridin-2-yl)methanol: To a stirred mixture of ethyl 3-(difluoromethoxy)picolinate (180 mg, 0.829 mmol, 1 equiv) in MeOH (3 mL) were added NaBH₄ (313 mg, 8.29 mmol, 10 equiv) in portions at 0° C. under nitrogen atmosphere. The resulting mixture was warmed to 60° C. and stirred for overnight. The desired product was detected by LCMS. The reaction was quenched by the addition of sat. NH₄Cl (aq.) (10 mL) at 0° C. The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with EtOAc/PE=1/1 to afford (3-(difluoromethoxy)pyridin-2-yl)methanol (100 mg, 68.8%) as a colorless oil.

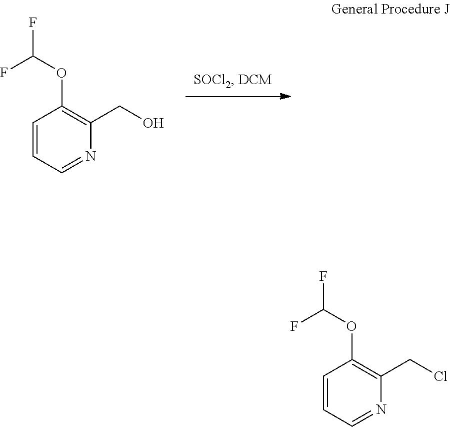 [SMILES: OCc1ncccc1OC(F)F ; FC(F)Oc1cccnc1CCl]

2-(Chloromethyl)-3-(difluoromethoxy)pyridine: To a stirred mixture of (3-(difluoromethoxy)pyridin-2-yl)methanol (100 mg, 0.571 mmol, 1 equiv) in DCM (3 mL) were added SOCl₂ (339 mg, 2.85 mmol, 5 equiv) dropwise at 0° C. under nitrogen atmosphere. The resulting mixture was stirred for 5 h at room temperature. The desired product was detected by LCMS. The resulting mixture was concentrated under reduced pressure. This resulted in 2-(chloromethyl)-3-(difluoromethoxy)pyridine (100 mg, crude) which was used to next step without further purification.

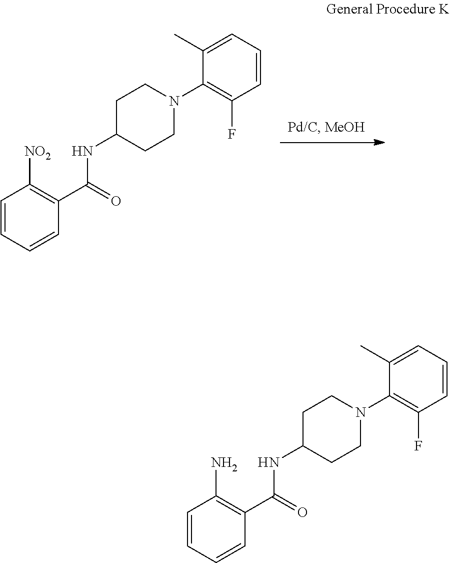 [SMILES: Cc1cccc(F)c1N1CCC(NC(=O)c2ccccc2[N+](=O)[O-])CC1 ; Cc1cccc(F)c1N1CCC(NC(=O)c2ccccc2N)CC1]

2-Amino-N-(1-(2-fluoro-6-methylphenyl)piperidin-4-yl)benzamide: To a solution of N-(1-(2-fluoro-6-methylphenyl)piperidin-4-yl)-2-nitrobenzamide (100 mg, 0.280 mmol, 1 equiv) in 5 mL MeOH was added Pd/C (30 mg, 10% Pd on carbon, wetted with water) in a 50 mL round-bottom flask. The mixture was hydrogenated at room temperature for 4 h under hydrogen atmosphere using a hydrogen balloon, filtered through a Celite® pad and concentrated under reduced pressure. This resulted in 2-amino-N-(1-(2-fluoro-6-methylphenyl)piperidin-4-yl)benzamide (70 mg, 76.4%) as a colorless oil.

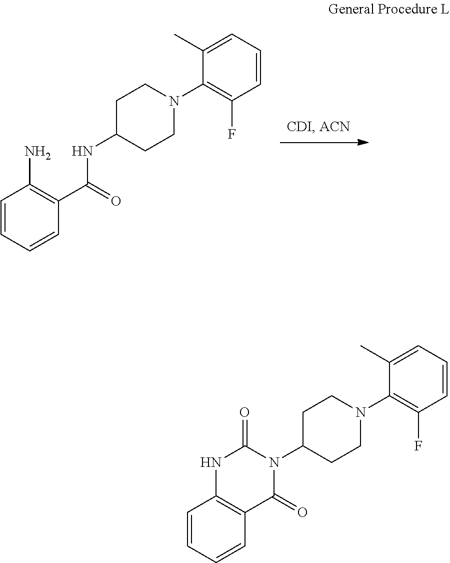 [SMILES: Cc1cccc(F)c1N1CCC(NC(=O)c2ccccc2N)CC1 ; Cc1cccc(F)c1N1CCC(n2c(=O)[nH]c3ccccc3c2=O)CC1]

3-(1-(2-Fluoro-6-methylphenyl)piperidin-4-yl)quinazoline-2,4(1H,3H)-dione: To a stirred solution of 2-amino-N-(1-(2-fluoro-6-methylphenyl)piperidin-4-yl)benzamide (70 mg, 0.214 mmol, 1 equiv) in ACN (2 mL) was added CDI (52 mg, 0.321 mmol, 1.5 equiv) at room temperature. The resulting mixture was stirred for overnight at room temperature. The desired product was detected by LCMS. The resulting mixture was concentrated under reduced pressure. The residue was purified by silica gel column chromatography, eluted with EtOAc/PE=1/1 to afford 3-(1-(2-fluoro-6-methylphenyl)piperidin-4-yl)quinazoline-2,4(1H,3H)-dione (60 mg, 79%) as a white solid.

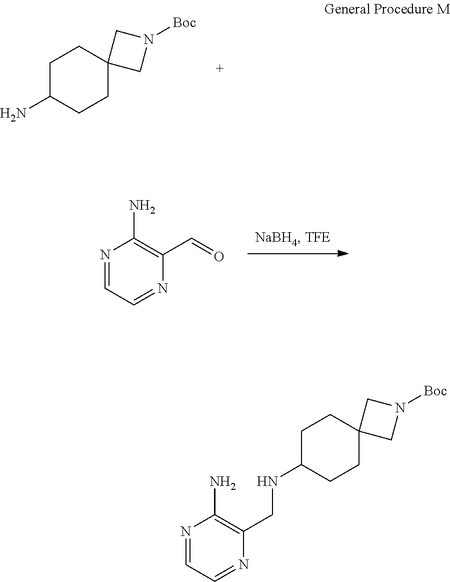 [SMILES: CC(C)(C)OC(=O)N1CC2(CCC(N)CC2)C1 ; Nc1nccnc1C=O ; CC(C)(C)OC(=O)N1CC2(CCC(NCc3nccnc3N)CC2)C1]

tert-Butyl 7-(((3-aminopyrazin-2-yl)methyl)amino)-2-azaspiro[3.5]nonane-2-carboxylate: A solution of tert-butyl 7-amino-2-azaspiro[3.5]nonane-2-carboxylate (100 mg, 0.416 mmol, 1 equiv) and 3-aminopyrazine-2-carbaldehyde (56.3 mg, 0.458 mmol, 1.1 equiv) in TFE (2 mL) was stirred for 1 h at 40° C. To the above mixture was added NaBH₄ (23.6 mg, 0.624 mmol, 1.5 equiv) at room temperature. The resulting mixture was stirred for additional overnight at 40° C. The desired product was detected by LCMS. The reaction was quenched by the addition of sat. NH₄Cl (aq.) (2 mL) at 0° C. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by Prep-TLC (EtOAc/PE=5/1) to afford tert-butyl 7-(((3-aminopyrazin-2-yl)methyl)amino)-2-azaspiro[3.5]nonane-2-carboxylate (100 mg, 69%) as a white solid.

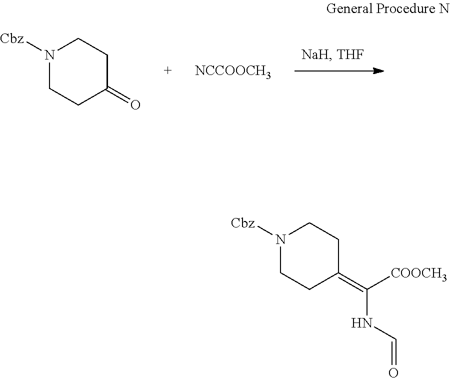

Benzyl 4-(1-formamido-2-methoxy-2-oxoethylidene)piperidine-1-carboxylate: To a stirred mixture of 2-methoxy-2-oxoacetonitrile (4.74 g, 55.7 mmol, 1.3 equiv) in THF (50 mL) was added NaH (2.57 g, 64.3 mmol, 1.5 equiv, 60%) in portions at 0° C. The resulting mixture was stirred for 45 min at 0° C. To the above mixture was added benzyl 4-oxopiperidine-1-carboxylate (10 g, 42.9 mmol, 1 equiv) in THF (5 mL) dropwise over 5 min at 0° C. The resulting mixture was stirred for additional 1 h at 0° C. The desired product was detected by LCMS. The reaction was quenched by the addition of Water (50 mL) at 0° C. The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with CH₂Cl₂/MeOH=10/1 to afford benzyl 4-(1-formamido-2-methoxy-2-oxoethylidene)piperidine-1-carboxylate (3 g, 31%) as an off-white solid.

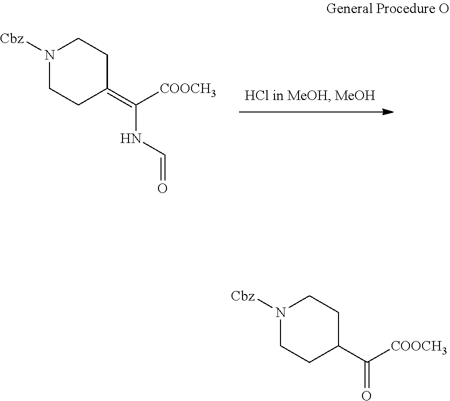 [SMILES: COC(=O)C(NC=O)=C1CCN(C(=O)OCc2ccccc2)CC1 ; COC(=O)C(=O)C1CCN(C(=O)OCc2ccccc2)CC1]

Benzyl 4-(2-methoxy-2-oxoacetyl)piperidine-1-carboxylate: To a stirred mixture of benzyl 4-(1-formamido-2-methoxy-2-oxoethylidene)piperidine-1-carboxylate (1 g, 3.01 mmol, 1 equiv) in MeOH (10 mL) were added HCl(g) in MeOH (2 mL, 4M) dropwise at room temperature under nitrogen atmosphere. The resulting mixture was stirred for 2 h at room temperature. The desired product was detected by LCMS. The resulting mixture was concentrated under reduced pressure. This resulted in benzyl 4-(2-methoxy-2-oxoacetyl)piperidine-1-carboxylate (1 g, crude) which was used in next step without further purification.

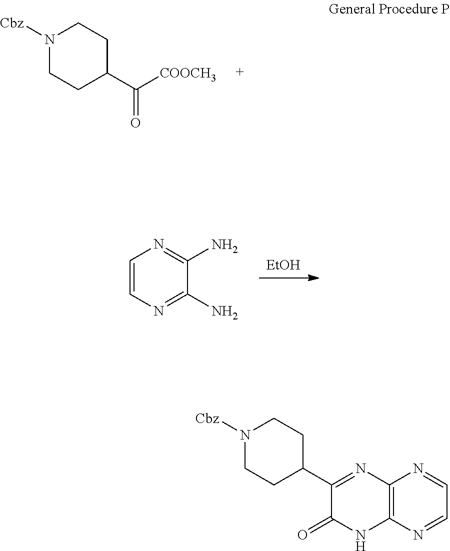 [SMILES: "EtOH" ; COC(=O)C(=O)C1CCN(C(=O)OCc2ccccc2)CC1 ; Nc1nccnc1N ; O=C(OCc1ccccc1)N1CCC(c2nc3nccnc3[nH]c2=O)CC1]

Benzyl 4-(3-oxo-3,4-dihydropyrazino[2,3-b]pyrazin-2-yl)piperidine-1-carboxylate: A solution of benzyl 4-(2-methoxy-2-oxoacetyl)piperidine-1-carboxylate (1 g, 3.27 mmol, 1 equiv) and pyrazine-2,3-diamine (0.36 g, 3.27 mmol, 1 equiv) in EtOH (10 mL) was stirred for overnight at 100° C. The desired product was detected by LCMS. The resulting mixture was concentrated under reduced pressure. The residue was purified by silica gel column chromatography, eluted with EtOAc/PE=1/1 to afford benzyl 4-(3-oxo-3,4-dihydropyrazino[2,3-b]pyrazin-2-yl)piperidine-1-carboxylate (250 mg, 20.8%) as a yellow solid.

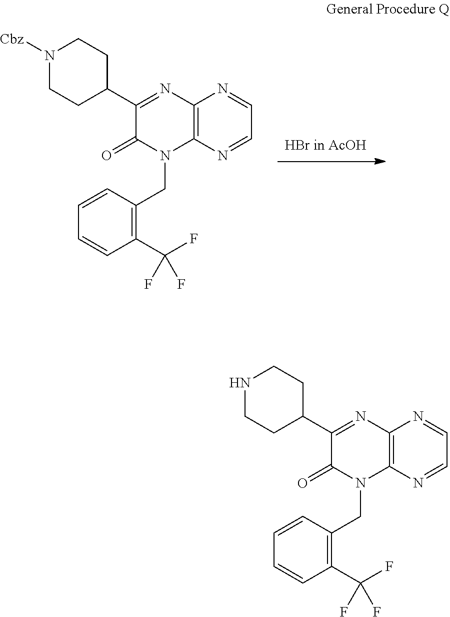 [SMILES: O=C(OCc1ccccc1)N1CCC(c2nc3nccnc3n(Cc3ccccc3C(F)(F)F)c2=O)CC1 ; O=c1c(C2CCNCC2)nc2nccnc2n1Cc1ccccc1C(F)(F)F]

3-(Piperidin-4-yl)-1-(2-(trifluoromethyl)benzyl)pyrazino[2,3-b]pyrazin-2(1H)-one: A solution of benzyl 4-(3-oxo-4-(2-(trifluoromethyl)benzyl)-3,4-dihydropyrazino[2,3-b]pyrazin-2-yl)piperidine-1-carboxylate (100 mg, 0.191 mmol, 1 equiv) in HBr in AcOH (33%, 3 mL) was stirred for 4 h at room temperature. The desired product was detected by LCMS. The resulting mixture was concentrated under reduced pressure. This resulted in 3-(piperidin-4-yl)-1-(2-(trifluoromethyl)benzyl)pyrazino[2,3-b]pyrazin-2(1H)-one (100 mg, crude) which was used in next step without further purification.

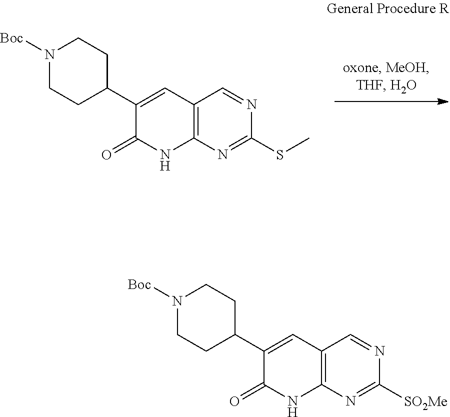 [SMILES: CSc1ncc2cc(C3CCN(C(=O)OC(C)(C)C)CC3)c(=O)[nH]c2n1 ; CC(C)(C)OC(=O)N1CCC(c2cc3cnc(S(C)(=O)=O)nc3[nH]c2=O)CC1]

tert-Butyl 4-(2-(methylsulfonyl)-7-oxo-7,8-dihydropyrido[2,3-d]pyrimidin-6-yl)piperidine-1-carboxylate: To a stirred solution of tert-butyl 4-(2-(methylthio)-7-oxo-7,8-dihydropyrido[2,3-d]pyrimidin-6-yl)piperidine-1-carboxylate (200 mg, 0.531 mmol, 1 equiv) in MeOH (2 mL), THF (2 mL) and H₂O (4 mL) was added potassium peroxymonosulfate (357 mg, 2.12 mmol, 4 equiv) at room temperature. The resulting mixture was stirred for overnight at room temperature under nitrogen atmosphere. The desired product was detected by LCMS. The precipitated solids were collected by filtration and washed with water (3×10 mL). The resulting solid was dried under vacuum. This resulted in tert-butyl 4-(2-(methylsulfonyl)-7-oxo-7,8-dihydropyrido[2,3-d]pyrimidin-6-yl)piperidine-1-carboxylate (200 mg, 92%) as a white solid.

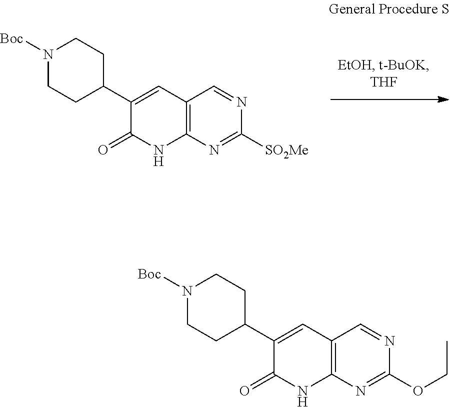 [SMILES: CC(C)(C)OC(=O)N1CCC(c2cc3cnc(S(C)(=O)=O)nc3[nH]c2=O)CC1 ; CCOc1ncc2cc(C3CCN(C(=O)OC(C)(C)C)CC3)c(=O)[nH]c2n1]

tert-Butyl 4-(2-ethoxy-7-oxo-7,8-dihydropyrido[2,3-d]pyrimidin-6-yl)piperidine-1-carboxylate: A solution of EtOH (50.7 mg, 1.1 mmol, 3 equiv) in THF (2 mL) was added t-BuOK (123 mg, 1.1 mmol, 3 equiv) at 0° C. The resulting mixture was stirred for 30 min. tert-butyl 4-(2-(methylsulfonyl)-7-oxo-7,8-dihydropyrido[2,3-d]pyrimidin-6-yl)piperidine-1-carboxylate (150 mg, 0.367 mmol, 1 equiv) was added and the mixture was allowed to warm to room temperature and stirred for 2 h. The desired product was detected by LCMS. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with EtOAc/PE=1/1 to afford tert-butyl 4-(2-ethoxy-7-oxo-7,8-dihydropyrido[2,3-d]pyrimidin-6-yl)piperidine-1-carboxylate (90 mg, 65%) as a white solid.

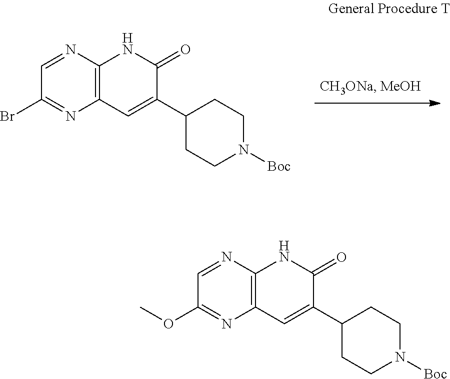 [SMILES: CC(C)(C)OC(=O)N1CCC(c2cc3nc(Br)cnc3[nH]c2=O)CC1 ; COc1cnc2[nH]c(=O)c(C3CCN(C(=O)OC(C)(C)C)CC3)cc2n1]

tert-Butyl 4-(2-methoxy-6-oxo-5,6-dihydropyrido[2,3-b]pyrazin-7-yl)piperidine-1-carboxylate: To a stirred solution of tert-butyl 4-(2-bromo-6-oxo-5,6-dihydropyrido[2,3-b]pyrazin-7-yl)piperidine-1-carboxylate (100 mg, 0.244 mmol, 1 equiv) in MeOH (2 mL) were added sodium methoxide (39.6 mg, 0.732 mmol, 3 equiv) at 25° C. The resulting mixture was stirred for 4 h at 100° C. The desired product was detected by LCMS. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with EtOAc/PE=1/1 to afford tert-butyl 4-(2-methoxy-6-oxo-5,6-dihydropyrido[2,3-b]pyrazin-7-yl)piperidine-1-carboxylate (60 mg, 68%) as a yellow solid.

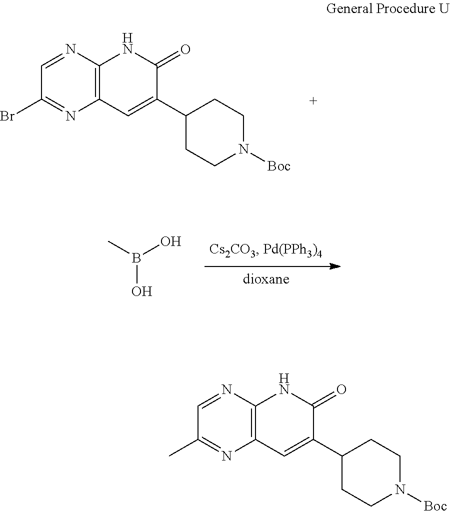

tert-Butyl 4-(2-methyl-6-oxo-5,6-dihydropyrido[2,3-b]pyrazin-7-yl)piperidine-1-carboxylate: To a stirred solution of tert-butyl 4-(2-bromo-6-oxo-5,6-dihydropyrido[2,3-b]pyrazin-7-yl)piperidine-1-carboxylate (40 mg, 0.098 mmol, 1 equiv) and methylboronic acid (8.78 mg, 0.147 mmol, 1.5 equiv) in dioxane (1 mL) were added Cs₂CO₃ (95.5 mg, 0.294 mmol, 3 equiv) and Pd(PPh₃)₄ (11.3 mg, 0.01 mmol, 0.1 equiv) at 25° C. under nitrogen atmosphere. The resulting mixture was stirred for 4 h at 100° C. under nitrogen atmosphere. The desired product was detected by LCMS. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by Prep-TLC (EtOAc/PE=3/1) to afford tert-butyl 4-(2-methyl-6-oxo-5,6-dihydropyrido[2,3-b]pyrazin-7-yl)piperidine-1-carboxylate (30 mg, 89%) as an off-white solid.

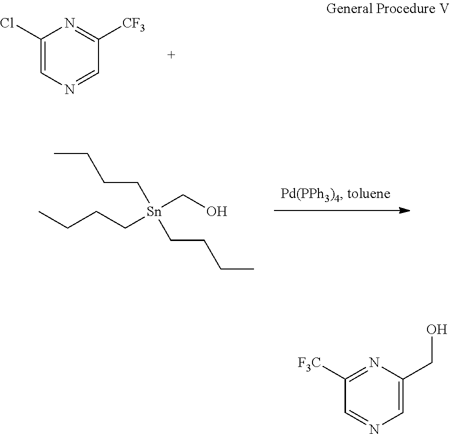

(6-(Trifluoromethyl)pyrazin-2-yl)methanol: To a stirred solution of 2-chloro-6-(trifluoromethyl)pyrazine (400 mg, 2.19 mmol, 1 equiv) and tetrakis(triphenylphosphine)palladium(0) (253 mg, 0.219 mmol, 0.1 equiv) in toluene (4 mL) was added (tributylstannyl)methanol (774 mg, 2.41 mmol, 1.1 equiv) at room temperature. The resulting mixture was stirred for overnight at 80° C. under nitrogen atmosphere. The desired product was detected by LCMS. The resulting mixture was concentrated under reduced pressure. The residue was purified by Prep-TLC (EtOAc/PE=5/1) to afford (6-(trifluoromethyl)pyrazin-2-yl)methanol (140 mg, 35%) as a colorless oil.

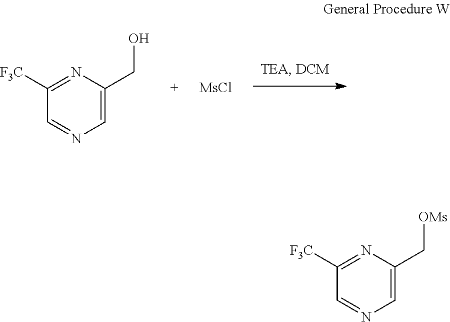 [SMILES: "MsCl" ; OCc1cncc(C(F)(F)F)n1 ; CS(=O)(=O)OCc1cncc(C(F)(F)F)n1]

(6-(Trifluoromethyl)pyrazin-2-yl)methyl methanesulfonate: To a stirred solution of (6-(trifluoromethyl)pyrazin-2-yl)methanol (140 mg, 0.786 mmol, 1 equiv) and TEA (238 mg, 2.35 mmol, 3 equiv) in DCM (2 mL) was added MsCl (135 mg, 1.17 mmol, 1.5 equiv) dropwise at 0° C. The resulting mixture was stirred for 2 h at room temperature. The desired product was detected by LCMS. The resulting mixture was concentrated under reduced pressure. The residue was purified by silica gel column chromatography, eluted with EtOAc/PE=1/1 to afford (6-(trifluoromethyl)pyrazin-2-yl)methyl methanesulfonate (60 mg, 29%) as a colorless oil.

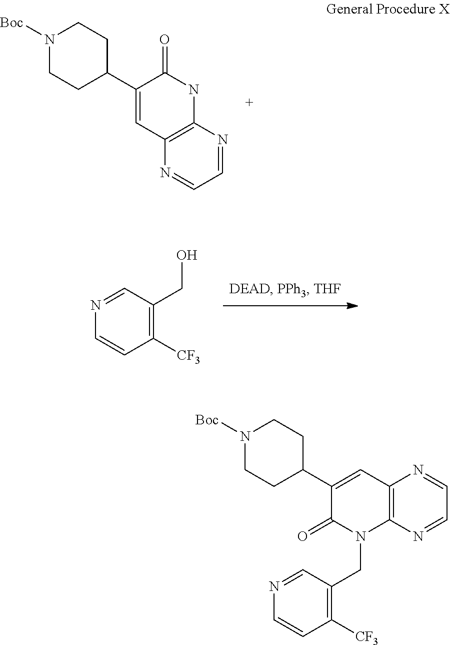 [SMILES: CC(C)(C)OC(=O)N1CCC(c2cc3nccnc3[nH]c2=O)CC1 ; OCc1cnccc1C(F)(F)F ; CC(C)(C)OC(=O)N1CCC(c2cc3nccnc3n(Cc3cnccc3C(F)(F)F)c2=O)CC1]

tert-Butyl 4-(6-oxo-5-((4-(trifluoromethyl)pyridin-3-yl)methyl)-5,6-dihydropyrido[2,3-b]pyrazine-7-yl)piperidine-1-carboxylate: To a stirred mixture of tert-butyl 4-(6-oxo-5,6-dihydropyrido[2,3-b]pyrazin-7-yl)piperidine-1-carboxylate (100 mg, 0.303 mmol, 1 equiv) and (4-(trifluoromethyl)pyridin-3-yl)methanol (58.9 mg, 0.333 mmol, 1.1 equiv) in THF (3 mL) were added PPh₃ (119 mg, 0.455 mmol, 1.5 equiv) and DEAD (79 mg, 0.455 mmol, 1.5 equiv) dropwise at 0° C. under air atmosphere. The resulting mixture was stirred for overnight at room temperature under nitrogen atmosphere. The resulting mixture was extracted with EtOAc (3×60 mL). The combined organic layers were washed with brine (2×60 mL), dried over anhydrous Na₂SO₄. After filtration, the filtrate was concentrated under reduced pressure. The residue was purified by silica gel column chromatography, eluted with EtOAc/PE=1/3 to afford tert-butyl 4-(6-oxo-5-((4-(trifluoromethyl)pyridin-3-yl)methyl)-5,6-dihydropyrido[2,3-b]pyrazine-7-yl) piperidine-1-carboxylate (87 mg, 58%) as a yellow oil.

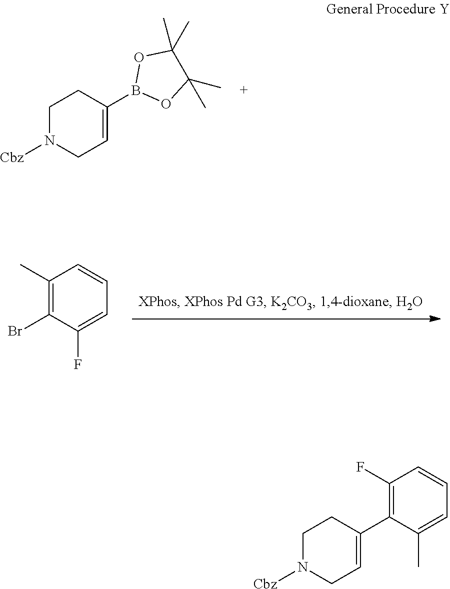

Benzyl 4-(2-fluoro-6-methylphenyl)-3,6-dihydropyridine-1(2H)-carboxylate: To a stirred mixture of benzyl 4-(4,4,5,5-tetramethyl-1,3,2-dioxaborolan-2-yl)-3,6-dihydro-2H-pyridine-1-carboxylate (2 g, 5.82 mmol, 1 equiv) and 2-bromo-1-fluoro-3-methylbenzene (1.32 g, 6.99 mmol, 1.2 equiv) in 1,4-dioxane (25 mL) and H₂O (5 mL) were added XPhos Pd G3 (0.49 g, 0.583 mmol, 0.1 equiv) and XPhos (0.28 g, 0.583 mmol, 0.1 equiv) and K₂CO₃ (2.42 g, 17.4 mmol, 3 equiv) at room temperature under air atmosphere. The resulting mixture was stirred for overnight at 80° C. under nitrogen atmosphere. The resulting mixture was concentrated under reduced pressure. The resulting mixture was extracted with EtOAc (3×200 mL). The combined organic layers were washed with brine (2×200 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 20% to 100% gradient in 25 min; detector, UV 254 nm, to afford benzyl 4-(2-fluoro-6-methylphenyl)-3,6-dihydro-2H-pyridine-1-carboxylate (1.6 g, 84%) as a colorless oil.

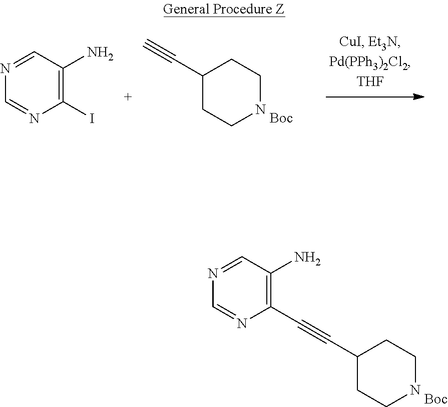

tert-Butyl 4-((5-aminopyrimidin-4-yl)ethynyl)piperidine-1-carboxylate: To a stirred solution of 4-iodopyrimidin-5-amine (200 mg, 0.905 mmol, 1 equiv) and tert-butyl 4-ethynylpiperidine-1-carboxylate (284 mg, 1.35 mmol, 1.5 equiv) in THF (5 mL) were added CuI (17.2 mg, 0.091 mmol, 0.1 equiv), Pd(PPh₃)₂Cl₂ (66.2 mg, 0.091 mmol, 0.1 equiv) and Et₃N (274 mg, 2.71 mmol, 3 equiv) at room temperature. The resulting mixture was stirred for overnight at 60° C. under argon atmosphere. The desired product was detected by LCMS. The resulting mixture was concentrated under reduced pressure. The resulting mixture was extracted with EtOAc (3×20 mL). The combined organic layers were washed with brine (1×50 mL), dried over anhydrous Na₂SO₄. After filtration, the filtrate was concentrated under reduced pressure. The residue was purified by silica gel column chromatography, eluted with EtOAc/PE=5/1 to afford tert-butyl 4-[2-(5-aminopyrimidin-4-yl)ethynyl]piperidine-1-carboxylate (200 mg, 73%) as a white solid.

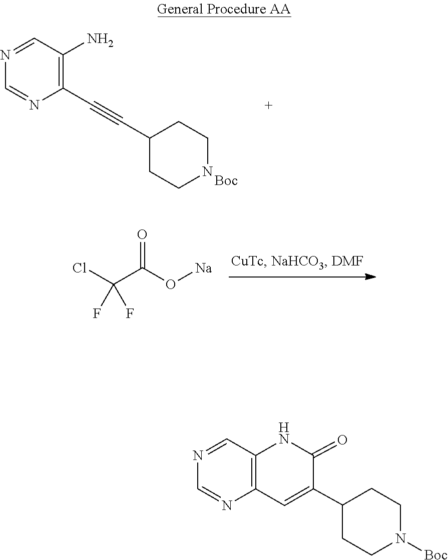

tert-butyl 4-(6-oxo-5,6-dihydropyrido[3,2-d]pyrimidin-7-yl)piperidine-1-carboxylate: To a stirred solution of tert-butyl 4-((5-aminopyrimidin-4-yl)ethynyl)piperidine-1-carboxylate (150 mg, 0.496 mmol, 1 equiv) and sodium 2-chloro-2,2-difluoroacetate (151 mg, 0.992 mmol, 2 equiv) in DMF (2 mL) were added CuTc (18.9 mg, 0.099 mmol, 0.2 equiv) and NaHCO₃ (83.3 mg, 0.992 mmol, 2 equiv) at room temperature. The resulting mixture was stirred for overnight at 100° C. under argon atmosphere. The desired product was detected by LCMS. The resulting mixture was extracted with EtOAc (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with EtOAc/PE=1/1 to afford tert-butyl 4-(6-oxo-5,6-dihydropyrido[3,2-d]pyrimidin-7-yl)piperidine-1-carboxylate (50 mg, 30%) as a white solid.

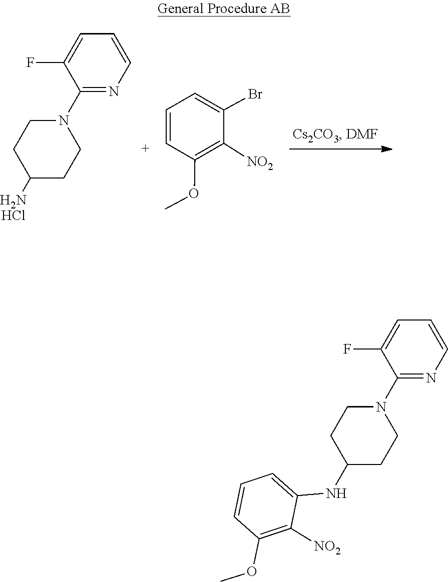

To a stirred solution of 1-(3-fluoropyridin-2-yl)piperidin-4-amine hydrochloride (60 mg, 0.259 mmol, 1 equiv) in DMF (1 mL) were added 1-fluoro-3-methoxy-2-nitrobenzene (48.8 mg, 0.285 mmol, 1.1 equiv) and Cs₂CO₃ (253 mg, 0.777 mmol, 3 equiv) at 25° C. The resulting mixture was stirred for 2 hours at 25° C. The resulting mixture was diluted with water (20 mL). The resulting mixture was extracted with EtOAc (2×20 mL). The combined organic layers were washed with brine (1×20 mL), dried over anhydrous sodium sulfate. After filtration, the filtrate was concentrated under reduced pressure. The residue was purified by silica gel column chromatography, eluted with EtOAc/PE=1/1 to afford 1-(3-fluoropyridin-2-yl)-N-(3-methoxy-2-nitrophenyl)piperidin-4-amine (60 mg, 66.7%) as a yellow solid.

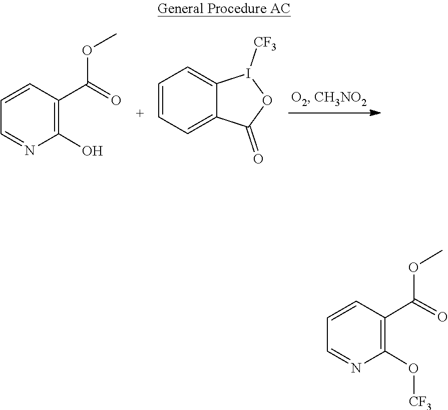

To a stirred solution of methyl 2-hydroxynicotinate (100 mg, 0.653 mmol, 1 equiv) in CH₃NO₂ (2 mL) was added 1-(trifluoromethyl)-1lambda3,2-benziodaoxol-3-one (619 mg, 1.96 mmol, 3 equiv) at room temperature. The resulting mixture was stirred for overnight at 100° C. under oxygen atmosphere. The desired product could be detected by LCMS. The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by silica gel column chromatography, eluted with EtOAc/PE=1/1 to afford methyl 2-(trifluoromethoxy)nicotinate (50 mg, 34.6%) as a colorless oil.

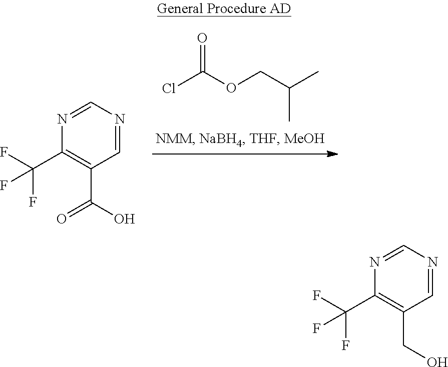

To a stirred solution of 4-(trifluoromethyl)pyrimidine-5-carboxylic acid (100 mg, 0.521 mmol, 1 equiv) in THF (1 mL) were added 4-methylmorpholine (94.7 mg, 0.938 mmol, 1.8 equiv) and isobutyl carbonochloridate (106 mg, 0.782 mmol, 1.5 equiv) at 0° C. The resulting mixture was stirred for 30 min at 0° C. To the mixture was added NaBH₄ (59 mg, 1.56 mmol, 3 equiv) and MeOH (0.4 mL) at 0° C. The resulting mixture was stirred for additional 1 h at 0° C. The resulting mixture was stirred for 1 h at room temperature. The desired product could be detected by LCMS. The reaction was quenched by the addition of water (2 mL) at 0° C. The resulting mixture was extracted with EtOAc (3×20 mL). The combined organic layers were washed with brine (1×10 mL), dried over anhydrous Na₂SO₄. After filtration, the filtrate was concentrated under reduced pressure. The residue was purified by Prep-TLC (PE/EA 1:1) to afford (4-(trifluoromethyl)pyrimidin-5-yl)methanol (35 mg, 37.7%) as a light yellow oil.

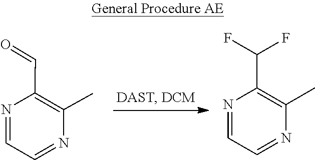

To a stirred solution of 3-methylpyrazine-2-carbaldehyde (200 mg, 1.63 mmol, 1 equiv) in DCM (2 mL) was added DAST (791 mg, 4.91 mmol, 3 equiv) dropwise at 0° C. The resulting mixture was stirred for overnight at room temperature. The desired product could be detected by LCMS. The residue was purified by silica gel column chromatography directly, eluted with PE/EA (1:1) to afford 2-(difluoromethyl)-3-methylpyrazine (60 mg, 25.4%) as a colorless oil.

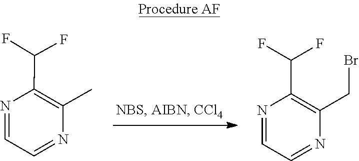

To a stirred solution of 2-(difluoromethyl)-3-methylpyrazine (100 mg, 0.694 mmol, 1 equiv) and NBS (185 mg, 1.04 mmol, 1.5 equiv) in CCl₄ (1 mL) was added AIBN (11.3 mg, 0.069 mmol, 0.1 equiv) at room temperature. The resulting mixture was stirred for overnight at 80° C. The desired product could be detected by LCMS. The resulting mixture was used in the next step directly without further purification.

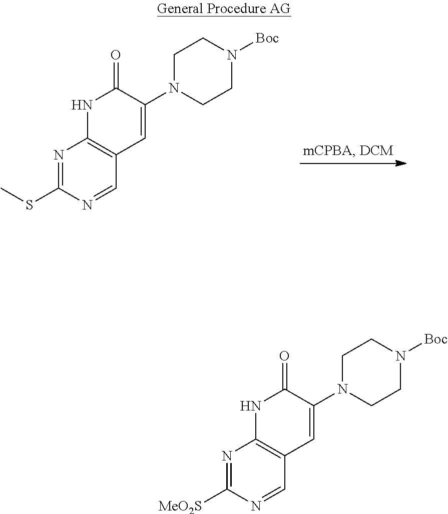 [SMILES: CSc1ncc2cc(N3CCN(C(=O)OC(C)(C)C)CC3)c(=O)[nH]c2n1 ; CC(C)(C)OC(=O)N1CCN(c2cc3cnc(S(C)(=O)=O)nc3[nH]c2=O)CC1]

To a stirred solution of tert-butyl 4-(2-(methylthio)-7-oxo-7,8-dihydropyrido[2,3-d]pyrimidin-6-yl)piperazine-1-carboxylate (800 mg, 2.11 mmol, 1 equiv) in DCM (5 mL) was added mCPBA (731 mg, 4.23 mmol, 2 equiv) in portions at 0° C. under air atmosphere. The resulting mixture was stirred for 30 min at room temperature under air atmosphere. The residue was purified by silica gel column chromatography, eluted with PE/EA (10% to 50% gradient in 10 min; detector, UV 254 nm) to afford tert-butyl 4-(2-(methylsulfonyl)-7-oxo-7,8-dihydropyrido[2,3-d]pyrimidin-6-yl)piperazine-1-carboxylate (300 mg, 34.5%) as a white solid.

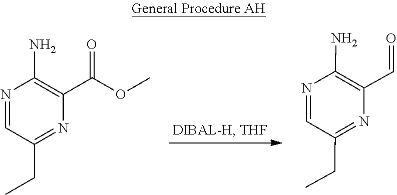

To a stirred mixture of 3-amino-6-ethylpyrazine-2-carboxylate (290 mg, 1.60 mmol, 1 equiv) in THF (3 mL) were added DIBAL-H (1M in THF, 2.4 mL, 2.4 mmol, 1.5 equiv) dropwise at 0° C. under nitrogen atmosphere. The resulting mixture was stirred for 1 h at 0° C. Desired product could be detected by LCMS. The reaction was quenched by the addition of Na₂SO₄·10H₂O at 0° C. The resulting mixture was filtered, the filter cake was washed with EtOAc (3×10 mL). The filtrate was concentrated under reduced pressure. This resulted in 3-amino-6-ethylpyrazine-2-carbaldehyde (180 mg, 69.2%) as a yellow solid.

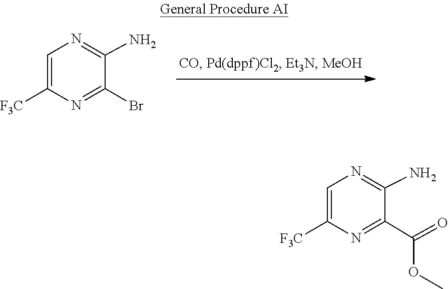

To a solution of 3-bromo-5-(trifluoromethyl)pyrazin-2-amine (450 mg, 1.86 mmol, 1 equiv) and Pd(dppf)Cl₂ (272 mg, 0.372 mmol, 0.2 equiv) in MeOH (5 mL) was added Et₃N (569 mg, 5.58 mmol, 3 equiv) in a pressure tank. The mixture was purged with nitrogen for 5 min and then was pressurized to 50 atm with carbon monoxide at 100° C. overnight. The reaction mixture was cooled to room temperature and filtered to remove insoluble solids. Desired product could be detected by LCMS. After removing the solvent, the residue was purified by silica gel column chromatography, eluted with EtOAc/PE=1/3 to afford methyl 3-amino-6-(trifluoromethyl)pyrazine-2-carboxylate (250 mg, 57.1%) as a light yellow oil.

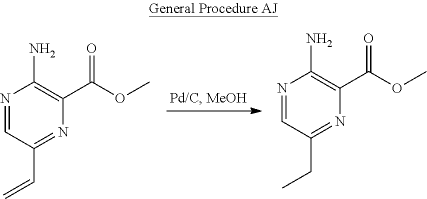

To a solution of methyl 3-amino-6-ethenylpyrazine-2-carboxylate (500 mg, 2.79 mmol, 1 equiv) in MeOH (10 mL) was added Pd/C (200 mg, 10% Pd on carbon, wetted with water) in a 50 mL round-bottom flask. The mixture was hydrogenated at room temperature for 4 h under hydrogen atmosphere using a hydrogen balloon, filtered through a Celite pad and concentrated under reduced pressure. This resulted in methyl 3-amino-6-ethylpyrazine-2-carboxylat (350 mg, 63.8%) as a yellow solid.

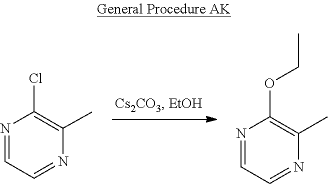

To a stirred solution of 2-chloro-3-methylpyrazine (500 mg, 3.9 mmol, 1.0 equiv) in EtOH (10 mL) were added Cs₂CO₃ (3.8 g, 11.7 mmol, 3 equiv) at 25° C. The resulting mixture was stirred for 4 h at 100° C. Desired product could be detected by LCMS. The resulting mixture was diluted with water (20 mL). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with EtOAc/PE=1/1 to afford 2-ethoxy-3-methylpyrazine (300 mg) as a yellow solid.

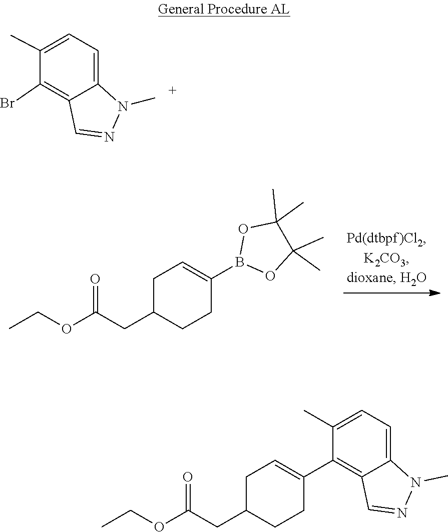

To a stirred solution of 4-bromo-1,5-dimethylindazole (500 mg, 2.22 mmol, 1 equiv) and ethyl 2-[4-(4,4,5,5-tetramethyl-1,3,2-dioxaborolan-2-yl)cyclohex-3-en-1-yl]acetate (653 mg, 2.22 mmol, 1.0 equiv) in dioxane (5 mL) and H₂O (1 mL) was added Pd(dtbpf)Cl₂ (145 mg, 0.222 mmol, 0.1 equiv) and K₂CO₃ (921 mg, 6.66 mmol, 3.0 equiv) at room temperature under argon atmosphere. The resulting mixture was stirred overnight at 80° C. under argon atmosphere. The reaction was monitored by LCMS. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:1) to afford ethyl 2-[4-(1,5-dimethylindazol-4-yl)cyclohex-3-en-1-yl]acetate (590 mg, 85%) as a light yellow oil.

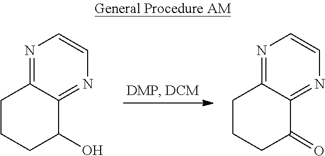 [SMILES: OC1CCCc2nccnc21 ; O=C1CCCc2nccnc21]

To a stirred solution of 5,6,7,8-tetrahydroquinoxalin-5-ol (850 mg, 5.63 mmol, 1.0 equiv.) in DCM (10 mL) was added DMP (4.7 g, 11.2 mmol, 2 equiv) at 0° C. The resulting mixture was stirred for 2 h at room temperature. Desired product could be detected by LCMS. The resulting mixture was used in the next step directly without further purification. The residue was purified by silica gel column chromatography, eluted with PE/EA (40% to 100% gradient in 10 min; detector, UV 254 nm.) to afford 7,8-dihydroquinoxalin-5(6H)-one (280 mg, 33.4%) as a white solid.

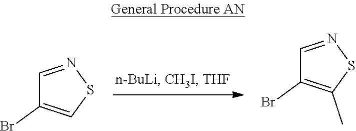

To a stirred mixture of 4-bromo-1,2-thiazole (500 mg, 3.05 mmol, 1 equiv) in THF (15 mL) was added n-BuLi (1.34 mL, 3.35 mmol, 1.1 equiv) dropwise at −78° C. under argon atmosphere. The resulting mixture was stirred for 2 h at −78° C. under argon atmosphere. To the above mixture was added methyl iodide (649 mg, 4.57 mmol, 1.5 equiv) dropwise at −78° C. under argon atmosphere. The resulting mixture was stirred for 2 h at −78° C. under argon atmosphere. The reaction was quenched by the addition of Na₂SO₄·10H₂O at 0° C. The residue was purified by silica gel column chromatography, eluted with PE/EA (0% to 100% gradient in 20 min) to afford 4-bromo-5-methyl-1,2-thiazole (220 mg, 40.5%) as a yellow oil.

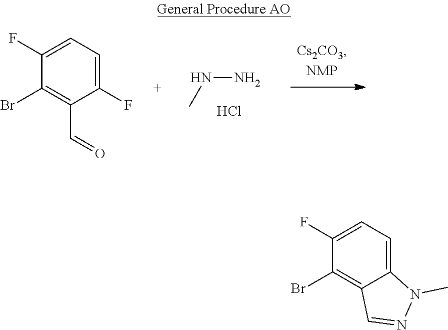

To a stirred solution of 2-bromo-3,6-difluorobenzaldehyde (250 mg, 1.13 mmol, 1 equiv) and methyl hydrazine hydrochloride (103 mg, 1.24 mmol, 1.1 equiv) in NMP (2 mL) was added Cs₂CO₃ (1106 mg, 3.39 mmol, 3 equiv) at room temperature under air atmosphere. The resulting mixture was stirred for overnight at 100° C. under air atmosphere. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (10% to 50% gradient in 20 min; detector, UV 254/280 nm.) to afford 4-bromo-5-fluoro-1-methylindazole (138 mg, 53.2%) as a yellow solid.

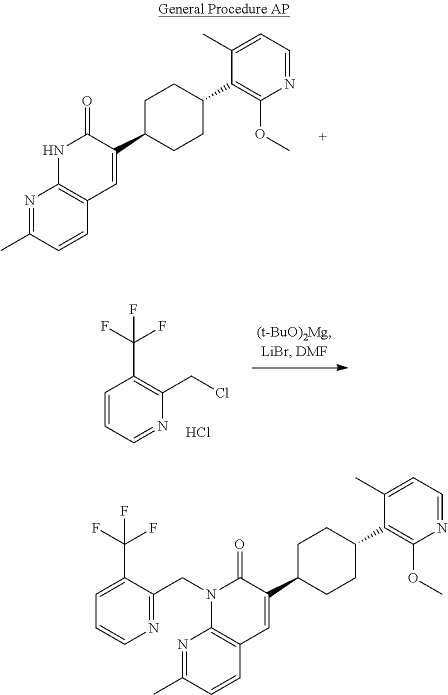

To a stirred mixture of 7-methyl-3-[(1r,4r)-4-(2-methoxy-4-methylpyridin-3-yl)cyclohexyl]-1H-1,8-naphthyridin-2-one (40 mg, 0.11 mmol, 1 equiv) and 2-(chloromethyl)-3-(trifluoromethyl)pyridine (25.8 mg, 0.132 mmol, 1.2 equiv) in DMF (1.5 mL) were added (t-BuO)₂Mg (93.8 mg, 0.55 mmol, 5 equiv) and LiBr (47.8 mg, 0.55 mmol, 5 equiv) at room temperature under air atmosphere. The resulting mixture was stirred for 4 h at 60° C. under air atmosphere. The resulting mixture was extracted with EtOAc (3×50 mL). The combined organic layers were washed with brine (1×60 mL), dried over anhydrous Na₂SO₄. After filtration, the filtrate was concentrated under reduced pressure. The residue was purified by Prep-TLC (n-hexane/EA 3:1) to afford 3-((1r,4r)-4-(2-methoxy-4-methylpyridin-3-yl)cyclohexyl)-7-methyl-1-((3-(trifluoromethyl)pyridin-2-yl)methyl)-1,8-naphthyridin-2(1H)-one (45.5 mg, 75.3%) as a white solid.

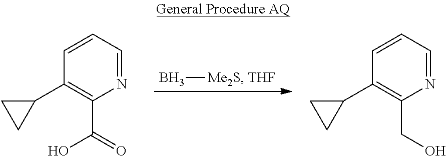

To a stirred solution of 3-cyclopropylpyridine-2-carboxylic acid (300 mg, 1.84 mmol, 1 equiv) in THF (5 mL) was added BH₃-Me₂S (698 mg, 9.20 mmol, 5.0 equiv) at 0° C. The resulting mixture was stirred for 2 h at 0° C. under air atmosphere. The reaction was monitored by LCMS. The resulting mixture was extracted with EtOAc (3×50 mL). The combined organic layers were washed with water (1×50 mL), dried over anhydrous Na₂SO₄. After filtration, the filtrate was concentrated under reduced pressure. The residue was purified by silica gel column chromatography, eluted with PE/EA (1:1) to afford (3-cyclopropylpyridin-2-yl)methanol (60 mg, 21.9%) as a colorless oil.

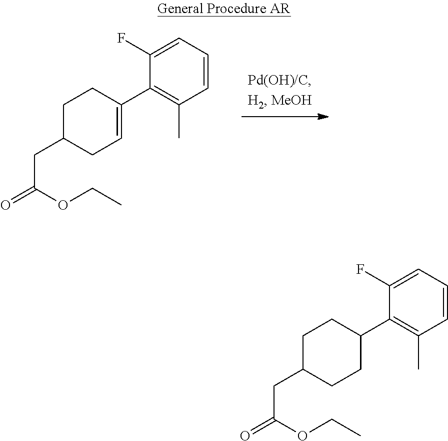 [SMILES: CCOC(=O)CC1CC=C(c2c(C)cccc2F)CC1 ; CCOC(=O)CC1CCC(c2c(C)cccc2F)CC1]

ethyl 2-(4-(2-fluoro-6-methylphenyl)cyclohexyl)acetate: A mixture of ethyl 2-[4-(2-fluoro-6-methylphenyl)cyclohex-3-en-1-yl]acetate (18 g, 65 mmol, 1 equiv) and Pd(OH)₂/C (6 g, 20% Pd(OH)₂ on carbon, wetted with water) in MeOH (200 mL) was stirred for 24 h at 50° C. under hydrogen atmosphere. Desired product could be detected by LCMS. The resulting mixture was filtered, the filter cake was washed with MeOH (3×100 mL). The filtrate was concentrated under reduced pressure. The crude product was used in the next step directly without further purification.

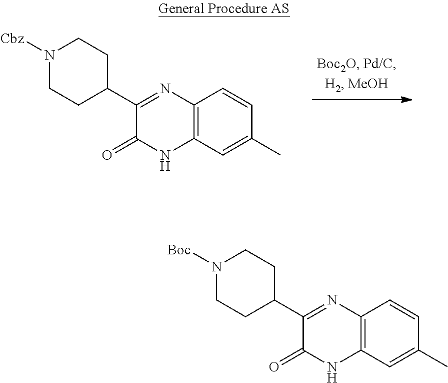 [SMILES: Cc1ccc2nc(C3CCN(C(=O)OCc4ccccc4)CC3)c(=O)[nH]c2c1 ; Cc1ccc2nc(C3CCN(C(=O)OC(C)(C)C)CC3)c(=O)[nH]c2c1]

To a solution of benzyl 4-(6-methyl-3-oxo-4H-quinoxalin-2-yl)piperidine-1-carboxylate (350 mg, 0.927 mmol, 1 equiv) and Boc₂O (202 mg, 0.927 mmol, 1 equiv) in 10 mL MeOH was added Pd/C (60%, 246 mg) in a pressure tank. The mixture was hydrogenated at room temperature under 30 psi of hydrogen pressure for 45 min, filtered through a Celite pad and concentrated under reduced pressure. This resulted in tert-butyl 4-(6-methyl-3-oxo-4H-quinoxalin-2-yl)piperidine-1-carboxylate (300 mg, 94.2%) as an off-white solid.

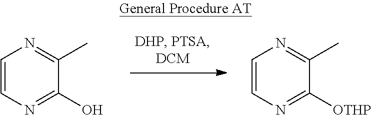

To a stirred solution of 3-methylpyrazin-2-ol (1 g, 9.08 mmol, 1.0 equiv) and DHP (2.29 g, 27.2 mmol, 3.0 equiv) in DCM (5 mL) was added PTSA (0.16 g, 0.908 mmol, 0.1 equiv) in portions at room temperature under air atmosphere. The resulting mixture was stirred overnight at room temperature. Desired product could be detected by LCMS. The resulting mixture was concentrated under reduced pressure. The residue was purified by silica gel column chromatography, eluted with PE/EA (1:10) to afford 2-methyl-3-(oxan-2-yloxy)pyrazine (870 mg, 49.3%) as a yellow oil.

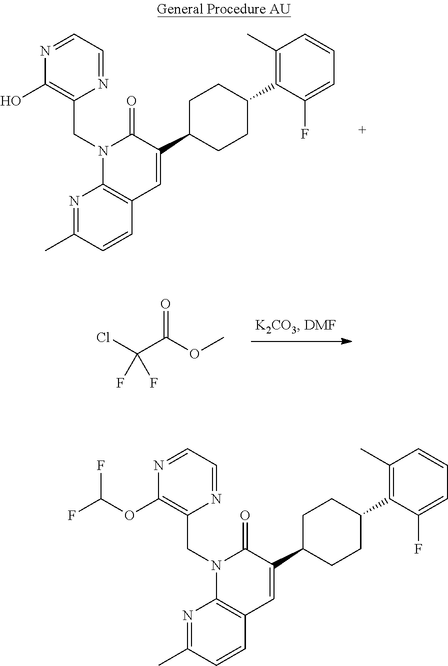

To a stirred solution of 1-[(3-hydroxypyrazin-2-yl)methyl]-7-methyl-3-[(1r,4r)-4-(2-fluoro-6-methylphenyl)cyclohexyl]-1,8-naphthyridin-2-one (150 mg, 0.327 mmol, 1.0 equiv) and methyl 2-chloro-2,2-difluoroacetate (56.72 mg, 0.392 mmol, 1.2 equiv) in DMF (3 mL) was added K₂CO₃ (136 mg, 0.981 mmol, 3.0 equiv) at room temperature under air atmosphere. Desired product could be detected by LCMS. The resulting mixture was extracted with EtOAc (3×20 mL). The combined organic layers were washed with brine (1×30 mL), dried over anhydrous Na₂SO₄. After filtration, the filtrate was concentrated under reduced pressure. The residue was purified by Prep-TLC (PE/EA=4:1) to afford 1-((3-(difluoromethoxy)pyrazin-2-yl)methyl)-3-((1r,4r)-4-(2-fluoro-6-methylphenyl)cyclohexyl)-7-methyl-1,8-naphthyridin-2(1H)-one (20.4 mg, 11.6%) as a white solid.

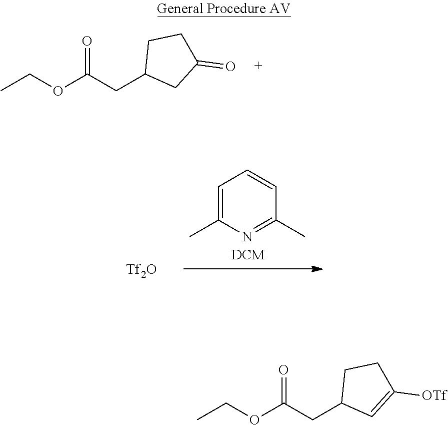

ethyl 2-(3-(((trifluoromethyl)sulfonyl)oxy)cyclopent-2-en-1-yl)acetate: To a stirred mixture of ethyl 2-(3-oxocyclopentyl)acetate (500 mg, 2.94 mmol, 1 equiv) in DCM (20 mL) was added lutidine (944 mg, 8.82 mmol, 3 equiv) dropwise at 0° C. under. The resulting mixture was stirred for 30 min at 0° C. To the above mixture was added Tf₂O (2.49 g, 8.82 mmol, 3 equiv) dropwise at 0° C. The resulting mixture was stirred for overnight at room temperature. The mixture was acidified to pH 6 with 1 mol/L HCl (aq.) at 0° C. The resulting mixture was extracted with DCM (3×120 mL). The combined organic layers were washed with brine (2×120 mL), dried over anhydrous Na₂SO₄. After filtration, the filtrate was concentrated under reduced pressure. The crude product was used in the next step directly without further purification.

### 7-(1-(2-Fluoro-6-methylphenyl)piperidin-4-yl)-5-(2-(trifluoromethyl)benzyl)pyrido[2,3-b]pyrazin-6(5H)-one (1)

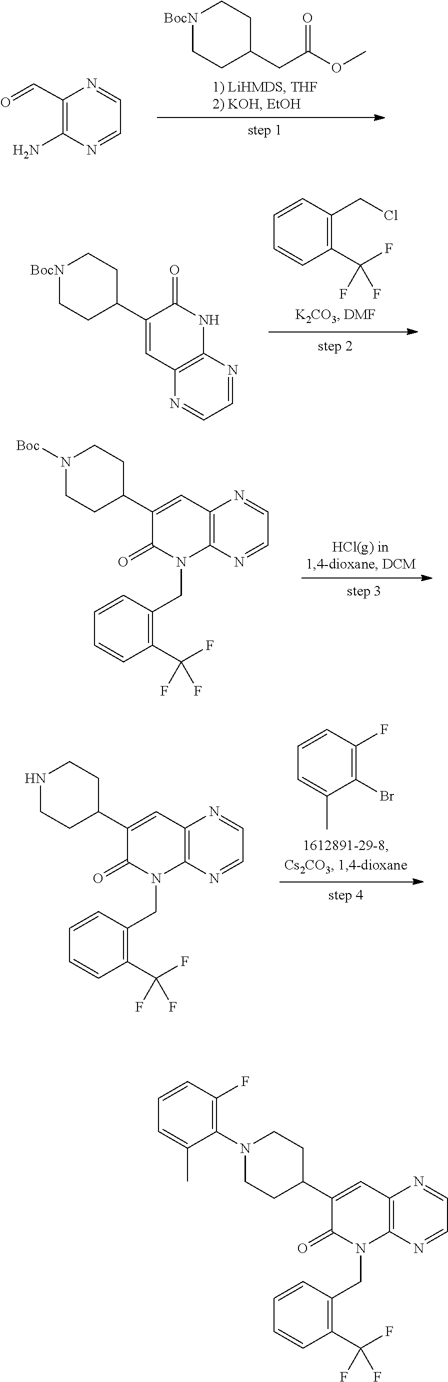

Step 1: tert-butyl 4-(6-oxo-5,6-dihydropyrido[2,3-b]pyrazin-7-yl)piperidine-1-carboxylate: Followed general procedure D using 3-aminopyrazine-2-carbaldehyde (600 mg, 4.87 mmol, 1 equiv) and tert-butyl 4-(2-methoxy-2-oxoethyl)piperidine-1-carboxylate (1880 mg, 7.30 mmol, 1.5 equiv) as the starting materials to give tert-butyl 4-(6-oxo-5,6-dihydropyrido[2,3-b]pyrazin-7-yl)piperidine-1-carboxylate (500 mg, 31%) as a light yellow solid. MS m/z: 331 [M+H]⁺.

Step 2: tert-butyl 4-(6-oxo-5-(2-(trifluoromethyl)benzyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (200 mg, 0.605 mmol, 1 equiv) and 1-(chloromethyl)-2-(trifluoromethyl)benzene (141 mg, 0.726 mmol, 1.2 equiv) as the starting materials to give tert-butyl 4-(6-oxo-5-(2-(trifluoromethyl)benzyl)-5,6-dihydropyrido[2,3-b]pyrazin-7-yl)piperidine-1-carboxylate (220 mg, 74%) as a yellow solid. MS m/z: 489 [M+H]⁺.

Step 3: 7-(piperidin-4-yl)-5-(2-(trifluoromethyl)benzyl)pyrido[2,3-b]pyrazin-6(5H)-one: Followed general procedure F using tert-butyl 4-(6-oxo-5-(2-(trifluoromethyl)benzyl)-5,6-dihydropyrido[2,3-b]pyrazin-7-yl)piperidine-1-carboxylate (220 mg, 0.45 mmol, 1 equiv) as the starting material to give the crude product 7-(piperidin-4-yl)-5-(2-(trifluoromethyl)benzyl)pyrido[2,3-b]pyrazin-6(5H)-one (180 mg) as a yellow oil. MS m/z: 389 [M+H]⁺.

Step 4: 7-(1-(2-fluoro-6-methylphenyl)piperidin-4-yl)-5-(2-(trifluoromethyl)benzyl)pyrido[2,3-b]pyrazin-6(5H)-one: Followed general procedure G using 7-(piperidin-4-yl)-5-(2-(trifluoromethyl)benzyl) pyrido[2,3-b]pyrazin-6(5H)-one (180 mg, 0.463 mmol, 1 equiv) and 2-bromo-1-fluoro-3-methyl benzene (105 mg, 0.556 mmol, 1.2 equiv) as the starting materials to give 7-(1-(2-fluoro-6-methylphenyl)piperidin-4-yl)-5-(2-(trifluoromethyl)benzyl)pyrido[2,3-b]pyrazin-6(5H)-one (9.8 mg, 4.0%) as a light yellow solid. ¹H NMR (400 MHz, Chloroform-d) δ 8.50 (d, 1H), 8.40 (d, 1H), 7.92 (s, 1H), 7.74-7.70 (m, 1H), 7.34-7.30 (m, 2H), 7.02-6.96 (m, 2H), 6.91-6.84 (m, 1H), 6.69-6.64 (m, 1H), 5.95 (s, 2H), 3.39-3.31 (m, 2H), 3.24-3.17 (m, 1H), 3.14-3.09 (m, 2H), 2.37 (s, 3H), 2.08-2.04 (m, 2H), 1.90-1.80 (m, 2H). MS m/z: 497.15 [M+H]⁺.

### 7-(1-(2-Fluoro-6-methylphenyl)piperidin-4-yl)-8-methyl-5-(2-(trifluoromethyl)benzyl)pyrido[2,3-b]pyrazin-6(5H)-one (2)

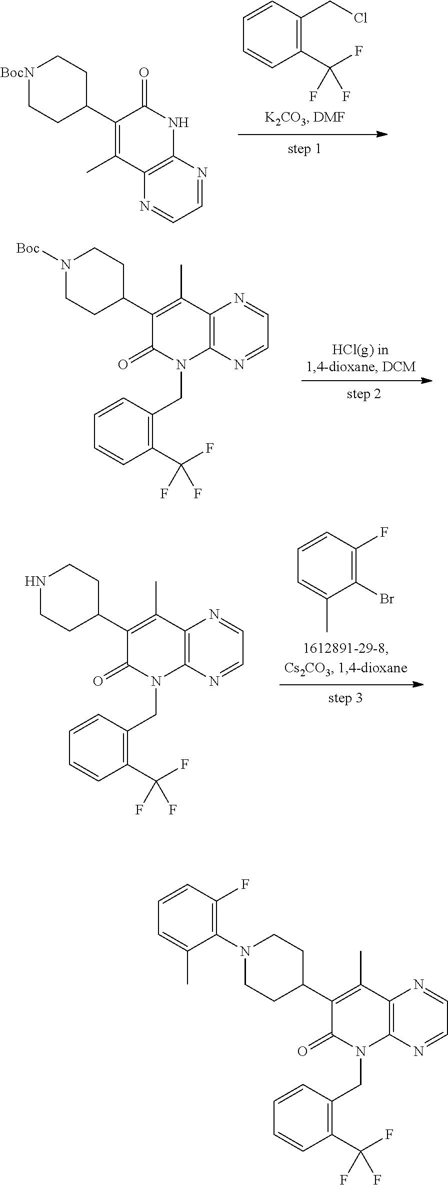

Step 1: tert-butyl 4-(8-methyl-6-oxo-5-(2-(trifluoromethyl)benzyl)-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (250 mg, 0.726 mmol, 1 equiv) and 1-(chloromethyl)-2-(trifluoromethyl)benzene (169 mg, 0.871 mmol, 1.2 equiv) as the starting materials to give tert-butyl 4-(8-methyl-6-oxo-5-(2-(trifluoromethyl)benzyl)-5,6-dihydropyrido[2,3-b]pyrazin-7-yl)piperidine-1-carboxylate (286 mg, 78%) as a yellow oil. MS m/z: 503 [M+H]⁺.

Step 2: 8-methyl-7-(piperidin-4-yl)-5-(2-(trifluoromethyl)benzyl)pyrido[2,3-b]pyrazin-6(5H)-one: Followed general procedure F using tert-butyl 4-(8-methyl-6-oxo-5-(2-(trifluoromethyl)benzyl)-5,6-dihydropyrido[2,3-b]pyrazin-7-yl)piperidine-1-carboxylate (286 mg, 0.569 mmol, 1 equiv) as the starting material to give the crude product 8-methyl-7-(piperidin-4-yl)-5-(2-(trifluoromethyl)benzyl)pyrido[2,3-b]pyrazin-6(5H)-one (210 mg) as a yellow oil. MS m/z: 403 [M+H]⁺.

Step 3: 7-(1-(2-fluoro-6-methylphenyl)piperidin-4-yl)-8-methyl-5-(2-(trifluoromethyl)benzyl) pyrido[2,3-b]pyrazin-6(5H)-one: Followed general procedure G using 8-methyl-7-(piperidin-4-yl)-5-(2-(trifluoromethyl) benzyl)pyrido[2,3-b]pyrazin-6(5H)-one (210 mg, 0.522 mmol, 1 equiv) and 2-bromo-1-fluoro-3-methylbenzene (118 mg, 0.626 mmol, 1.2 equiv) as the starting materials to give 7-(1-(2-fluoro-6-methylphenyl)piperidin-4-yl)-8-methyl-5-(2-(trifluoromethyl)benzyl) pyrido[2,3-b]pyrazin-6(5H)-one (62.2 mg, 22.8%) as an off-white solid. ¹H NMR (400 MHz, Chloroform-d) δ 8.49 (d, 1H), 8.35 (d, 1H), 7.72-7.68 (m, 1H), 7.33-7.28 (m, 2H), 7.00-6.93 (m, 2H), 6.89-6.83 (m, 1H), 6.69-6.66 (m, 1H), 5.92 (s, 2H), 3.37-3.26 (m, 3H), 3.11 (d, 2H), 2.78 (s, 3H), 2.77-2.70 (m, 2H), 2.38 (s, 3H), 1.63 (d, 2H). MS m/z: 511.25 [M+H]⁺.

### 7-(1-(2-Fluoro-6-methylphenyl)piperidin-4-yl)-5-((3-methoxypyrazin-2-yl)methyl)-8-methyl pyrido[2,3-b]pyrazin-6(5H)-one (3)

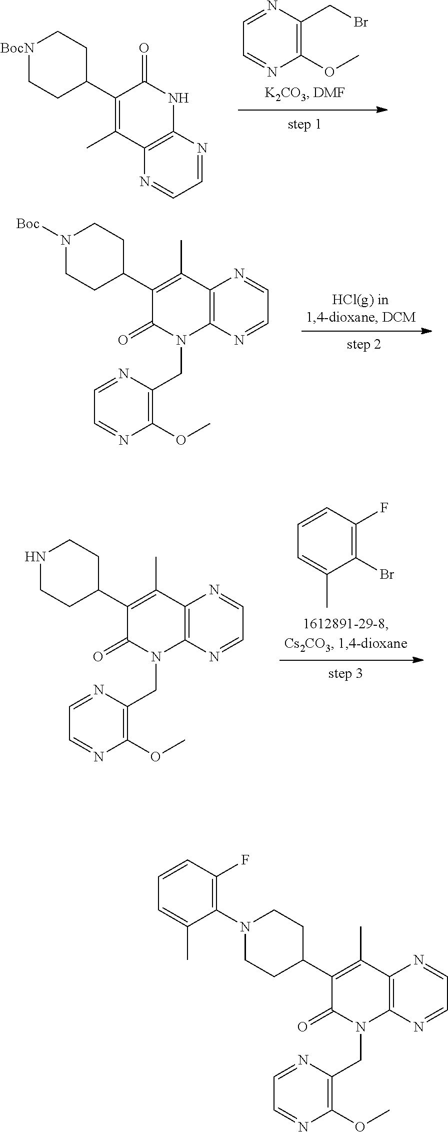

Step 1: tert-butyl 4-(5-((3-methoxypyrazin-2-yl)methyl)-8-methyl-6-oxo-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (140 mg, 0.406 mmol, 1 equiv) and 2-(bromomethyl)-3-methoxypyrazine (98 mg, 0.487 mmol, 1.2 equiv) as the starting materials to give tert-butyl 4-(5-((3-methoxypyrazin-2-yl)methyl)-8-methyl-6-oxo-5,6-dihydropyrido[2,3-b]pyrazin-7-yl)piperidine-1-carboxylate (168 mg, 88%) as a yellow solid. MS m/z: 467 [M+H]⁺.

Step 2: 5-((3-methoxypyrazin-2-yl)methyl)-8-methyl-7-(piperidin-4-yl)pyrido[2,3-b]pyrazin-6(5H)-one: Followed general procedure F using tert-butyl 4-(5-((3-methoxypyrazin-2-yl)methyl)-8-methyl-6-oxo-5,6-dihydropyrido[2,3-b]pyrazin-7-yl)piperidine-1-carboxylate (168, 0.36 mmol, 1 equiv) as the starting material to give the crude product 5-((3-methoxypyrazin-2-yl)methyl)-8-methyl-7-(piperidin-4-yl)pyrido[2,3-b]pyrazin-6(5H)-one (100 mg) as a yellow oil. MS m/z: 367 [M+H]⁺.

Step 3: 7-(1-(2-fluoro-6-methylphenyl)piperidin-4-yl)-5-((3-methoxypyrazin-2-yl)methyl)-8-methylpyrido[2,3-b]pyrazin-6(5H)-one: Followed general procedure G using 5-((3-methoxypyrazin-2-yl)methyl)-8-methyl-7-(piperidin-4-yl)pyrido[2,3-b]pyrazin-6(5H)-one (100 mg, 0.273 mmol, 1 equiv) and 2-bromo-1-fluoro-3-methylbenzene (61.9 mg, 0.328 mmol, 1.2 equiv) as the starting materials to give 7-(1-(2-fluoro-6-methylphenyl)piperidin-4-yl)-5-((3-methoxypyrazin-2-yl)methyl)-8-methylpyrido[2,3-b]pyrazin-6(5H)-one (22.6 mg, 16.7%) as an off-white solid. ¹H NMR (300 MHz, Chloroform-d) δ 8.46 (d, 1H), 8.31 (d, 1H), 7.91 (d, 1H), 7.80 (d, 1H), 6.97-6.94 (m, 2H), 6.91-6.81 (m, 1H), 5.80 (s, 2H), 4.06 (s, 3H), 3.33-3.22 (m, 3H), 3.07 (d, 2H), 2.75-2.64 (m, 5H), 2.36 (s, 3H), 1.62 (d, 2H). MS m/z: 475.25 [M+H]⁺.

### 7-(1-(2-Fluoro-6-methylphenyl)piperidin-4-yl)-8-methyl-5-((3-(2,2,2-trifluoroethoxy)pyrazin-2-yl)methyl)pyrido[2,3-b]pyrazin-6(5H)-one (4)

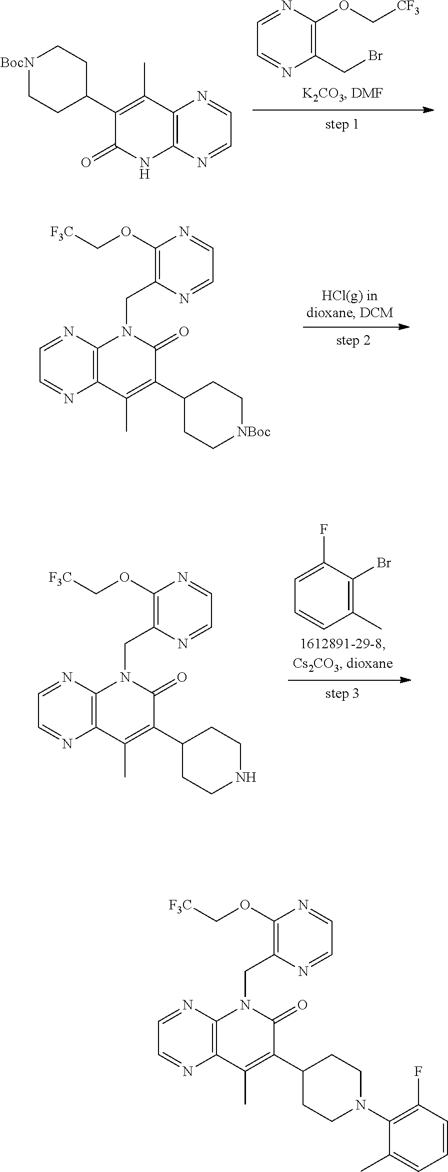

Step 1: tert-butyl 4-(8-methyl-6-oxo-5-((3-(2,2,2-trifluoroethoxy)pyrazin-2-yl)methyl)-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (140 mg, 0.406 mmol, 1 equiv) and 2-(bromomethyl)-3-(2,2,2-trifluoroethoxy)pyrazine (131 mg, 0.487 mmol, 1.2 equiv) as the starting materials to give tert-butyl 4-(8-methyl-6-oxo-5-((3-(2,2,2-trifluoroethoxy)pyrazin-2-yl)methyl)-5,6-dihydropyrido[2,3-b]pyrazin-7-yl)piperidine-1-carboxylate (214 mg, 98%) as a yellow solid. MS m/z: 535 [M+H]⁺.

Step 2: 5-(2-fluorobenzyl)-7-(piperidin-4-yl)pyrido[2,3-b]pyrazin-6(5H)-one: Followed general procedure F using tert-butyl 4-(8-methyl-6-oxo-5-((3-(2,2,2-trifluoroethoxy)pyrazin-2-yl)methyl)-5,6-dihydropyrido[2,3-b]pyrazin-7-yl)piperidine-1-carboxylate (150 mg, 0.281 mmol, 1 equiv) as the starting material to give the crude product 8-methyl-7-(piperidin-4-yl)-5-((3-(2,2,2-trifluoroethoxy)pyrazin-2-yl)methyl)pyrido[2,3-b]pyrazin-6(5H)-one (100 mg) as an off white solid. MS m/z 435 [M+H]⁺.

Step 3: 7-(1-(2-fluoro-6-methylphenyl)piperidin-4-yl)-8-methyl-5-((3-(2,2,2-trifluoroethoxy)pyrazin-2-yl)methyl)pyrido[2,3-b]pyrazin-6(5H)-one: Followed general procedure G using 8-methyl-7-(piperidin-4-yl)-5-((3-(2,2,2-trifluoroethoxy)pyrazin-2-yl)methyl)pyrido[2,3-b]pyrazin-6(5H)-one (100 mg, 0.231 mmol, 1 equiv) and 2-bromo-1-fluoro-3-methylbenzene (52.4 mg, 0.277 mmol, 1.2 equiv) as the starting materials to give 7-(1-(2-fluoro-6-methylphenyl)piperidin-4-yl)-8-methyl-5-((3-(2,2,2-trifluoroethoxy)pyrazin-2-yl)methyl)pyrido[2,3-b]pyrazin-6(5H)-one (42 mg, 31.5%) as a yellow solid. ¹H NMR (300 MHz, Chloroform-d) δ 8.50 (d, 1H), 8.34 (d, 1H), 8.04-7.90 (m, 2H), 7.05-6.95 (m, 2H), 6.94-6.84 (m, 1H), 5.89 (s, 2H), 4.87 (q, 2H), 3.41-3.22 (m, 3H), 3.17-3.08 (m, 2H), 2.85-2.66 (m, 5H), 2.41 (s, 3H), 1.66 (d, 2H). MS m/z: 543.25 [M+H]⁺.

### 7-(1-(2-Fluoro-6-methylphenyl)piperidin-4-yl)-5-((3-methoxypyrazin-2-yl)methyl)pyrido[2,3-b]pyrazin-6(5H)-one (5)

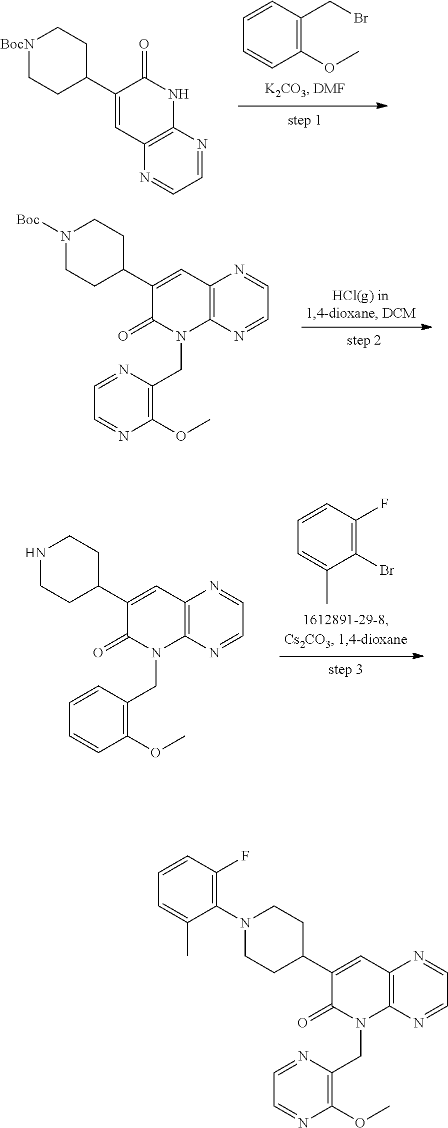

Step 1: tert-butyl 4-(5-((3-methoxypyrazin-2-yl)methyl)-6-oxo-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (110 mg, 0.333 mmol, 1 equiv) and 1-(bromomethyl)-2-methoxybenzene (73.6 mg, 0.366 mmol, 1.1 equiv) as the starting materials to give tert-butyl 4-(5-((3-methoxypyrazin-2-yl)methyl)-6-oxo-5,6-dihydropyrido[2,3-b]pyrazin-7-yl)piperidine-1-carboxylate (116 mg, 77%) as a light yellow solid. MS m/z: 453 [M+H]⁺.

Step 2: 5-(2-methoxybenzyl)-7-(piperidin-4-yl)pyrido[2,3-b]pyrazin-6(5H)-one: Followed general procedure F using tert-butyl 4-(5-((3-methoxypyrazin-2-yl)methyl)-6-oxo-5,6-dihydropyrido[2,3-b]pyrazin-7-yl)piperidine-1-carboxylate (116 mg, 0.256 mmol, 1 equiv) as the starting material to give the crude product 5-(2-methoxybenzyl)-7-(piperidin-4-yl)pyrido[2,3-b]pyrazin-6(5H)-one (65 mg) as a yellow oil. MS m/z: 353 [M+H]⁺.

Step 3: 7-(1-(2-fluoro-6-methylphenyl)piperidin-4-yl)-5-((3-methoxypyrazin-2-yl)methyl)pyrido[2,3-b]pyrazin-6(5H)-one: Followed general procedure G using 5-(2-methoxybenzyl)-7-(piperidin-4-yl)pyrido[2,3-b]pyrazin-6(5H)-one (65 mg, 0.185 mmol, 1 equiv) and 2-bromo-1-fluoro-3-methylbenzene (42 mg, 0.222 mmol, 1.2 equiv) as the starting materials to give 7-(1-(2-fluoro-6-methylphenyl)piperidin-4-yl)-5-((3-methoxypyrazin-2-yl)methyl)pyrido[2,3-b]pyrazin-6(5H)-one (22.1 mg, 25.7%) as a white solid. ¹H NMR (400 MHz, Chloroform-d) δ 8.46 (d, 1H), 8.36 (d, 1H), 7.93-7.88 (m, 2H), 7.81 (d, 1H), 7.01-6.96 (m, 2H), 6.90-6.84 (m, 1H), 5.83 (s, 2H), 4.08 (s, 3H), 3.38-3.32 (m, 2H), 3.21-3.10 (m, 3H), 2.38 (s, 3H), 2.07-2.01 (m, 2H), 1.91-1.81 (m, 2H). MS m/z: 461.20 [M+H]⁺.

### 7-(1-(2-Fluoro-6-methylphenyl)piperidin-4-yl)-5-((3-(2,2,2-trifluoroethoxy)pyrazin-2-yl) methyl)pyrido[2,3-b]pyrazin-6(5H)-one (6)

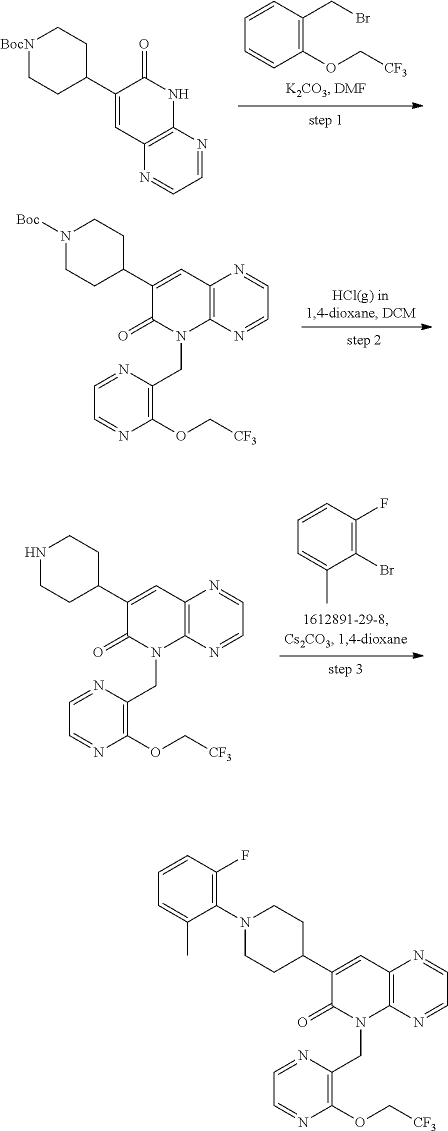

Step 1: tert-butyl 4-(6-oxo-5-((3-(2,2,2-trifluoroethoxy)pyrazin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (100 mg, 0.303 mmol, 1 equiv) and 1-(bromomethyl)-2-(2,2,2-trifluoroethoxy)benzene (89.5 mg, 0.333 mmol, 1.1 equiv) as the starting materials to give tert-butyl 4-(6-oxo-5-((3-(2,2,2-trifluoroethoxy)pyrazin-2-yl)methyl)-5,6-dihydropyrido[2,3-b]pyrazine-7-yl)piperidine-1-carboxylate (150 mg, 95%) as a light yellow solid. MS m/z: 521 [M+H]⁺.

Step 2: 7-(piperidin-4-yl)-5-((3-(2,2,2-trifluoroethoxy)pyrazin-2-yl)methyl)pyrido[2,3-b]pyrazin-6(5H)-one: Followed general procedure F using tert-butyl 4-(6-oxo-5-((3-(2,2,2-trifluoroethoxy) pyrazin-2-yl)methyl)-5,6-dihydropyrido[2,3-b]pyrazine-7-yl)piperidine-1-carboxylate (150 mg, 0.288 mmol, 1 equiv) as the starting material to give the crude product 7-(piperidin-4-yl)-5-((3-(2,2,2-trifluoroethoxy)pyrazin-2-yl)methyl)pyrido[2,3-b]pyrazin-6(5H)-one (110 mg) as a yellow oil. MS m/z: 421 [M+H]⁺.

Step 3: 7-(1-(2-fluoro-6-methylphenyl)piperidin-4-yl)-5-((3-(2,2,2-trifluoroethoxy)pyrazin-2-yl)methyl)pyrido[2,3-b]pyrazin-6(5H)-one: Followed general procedure G using 7-(piperidin-4-yl)-5-((3-(2,2,2-trifluoroethoxy) pyrazin-2-yl)methyl)pyrido[2,3-b]pyrazin-6(5H)-one (108 mg, 0.257 mmol, 1 equiv) and 2-bromo-1-fluoro-3-methylbenzene (58.2 mg, 0.308 mmol, 1.2 equiv) as the starting materials to give 7-(1-(2-fluoro-6-methylphenyl)piperidin-4-yl)-5-((3-(2,2,2-trifluoroethoxy)pyrazin-2-yl) methyl)pyrido[2,3-b]pyrazin-6(5H)-one (43.5 mg, 31.7%) as a white solid. ¹H NMR (400 MHz, Chloroform-d) δ 8.47 (d, 1H), 8.36 (d, 1H), 7.96-7.93 (m, 2H), 7.88 (d, 1H), 7.01-6.96 (m, 2H), 6.91-6.84 (m, 1H), 5.90 (s, 2H), 4.85 (q, 2H), 3.38-3.30 (m, 2H), 3.21-3.10 (m, 3H), 2.37 (s, 3H), 2.06-2.02 (m, 2H), 1.89-1.80 (m, 2H). MS m/z: 529.25 [M+H]⁺.

### 5-(2-Fluorobenzyl)-8-methyl-7-(1-(2-(trifluoromethyl)phenyl)piperidin-4-yl)pyrido[2,3-b]pyrazin-6(5H)-one (7)

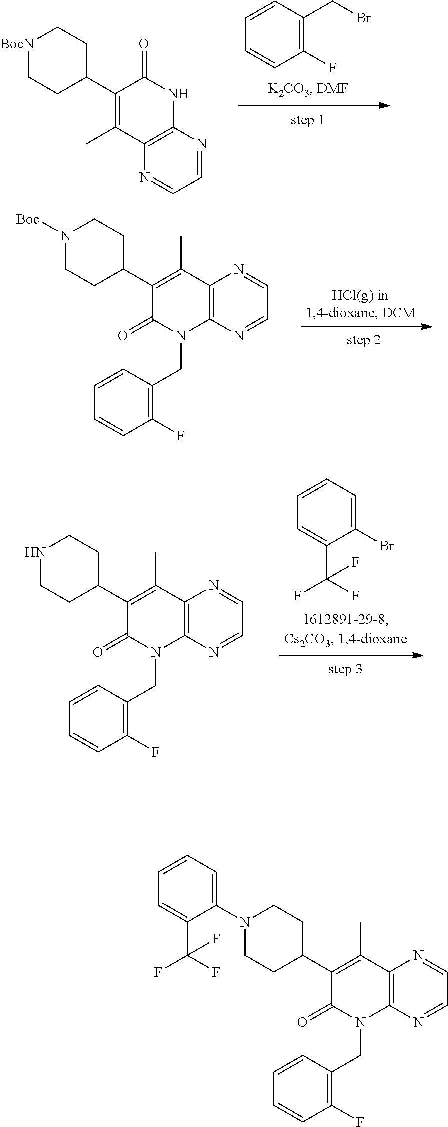

Step 1: tert-butyl 4-(5-(2-fluorobenzyl)-8-methyl-6-oxo-5,6-dihydropyrido[2,3-b]pyrazin-7-yl) piperidine-1-carboxylate: Followed general procedure E using tert-butyl 4-(8-methyl-6-oxo-5,6-dihydropyrido[2,3-b]pyrazin-7-yl)piperidine-1-carboxylate (170 mg, 0.494 mmol, 1 equiv) and 1-(bromomethyl)-2-fluorobenzene (139 mg, 0.741 mmol, 1.5 equiv) as the starting materials to give tert-butyl 4-(5-(2-fluorobenzyl)-8-methyl-6-oxo-5,6-dihydropyrido[2,3-b]pyrazin-7-yl) piperidine-1-carboxylate (200 mg, 89%) as a yellow solid. MS m/z: 453 [M+H]⁺.

Step 2: 5-(2-fluorobenzyl)-8-methyl-7-(piperidin-4-yl)pyrido[2,3-b]pyrazin-6(5H)-one: Followed general procedure F using tert-butyl 4-(5-(2-fluorobenzyl)-8-methyl-6-oxo-5,6-dihydropyrido[2,3-b]pyrazin-7-yl) piperidine-1-carboxylate (200 mg, 0.442 mmol, 1 equiv) as the starting material to give the crude product 5-(2-fluorobenzyl)-8-methyl-7-(piperidin-4-yl)pyrido[2,3-b]pyrazin-6(5H)-one (151 mg) as a yellow oil. MS m/z: 353 [M+H]⁺.

Step 3: 5-(2-fluorobenzyl)-8-methyl-7-(1-(2-(trifluoromethyl)phenyl)piperidin-4-yl)pyrido[2,3-b]pyrazin-6(5H)-one: Followed general procedure G using 5-(2-fluorobenzyl)-8-methyl-7-(piperidin-4-yl)pyrido[2,3-b]pyrazin-6(5H)-one (70 mg, 0.199 mmol, 1 equiv) and 1-bromo-2-(trifluoromethyl)benzene (53.6 mg, 0.239 mmol, 1.2 equiv) as the starting materials to give 5-(2-fluorobenzyl)-8-methyl-7-(1-(2-(trifluoromethyl)phenyl)piperidin-4-yl)pyrido[2,3-b]pyrazin-6(5H)-one (50.5 mg, 50.9%) as an off-white solid. ¹H NMR (400 MHz, Chloroform-d) δ 8.49 (d, 1H), 8.41 (d, 1H), 7.67-7.53 (m, 3H), 7.24-7.17 (m, 2H), 7.08-7.03 (m, 1H), 6.99-6.90 (m, 2H), 5.78 (s, 2H), 3.57 (s, 1H), 3.29 (d, 2H), 3.04 (t, 2H), 2.79 (s, 5H), 1.72 (d, 2H). MS m/z: 497.25 [M+H]⁺.

### 7-(1-(3-Methylpyrazin-2-yl)piperidin-4-yl)-5-(2-(trifluoromethyl)benzyl)pyrido[2,3-b]pyrazin-6(5H)-one (8)

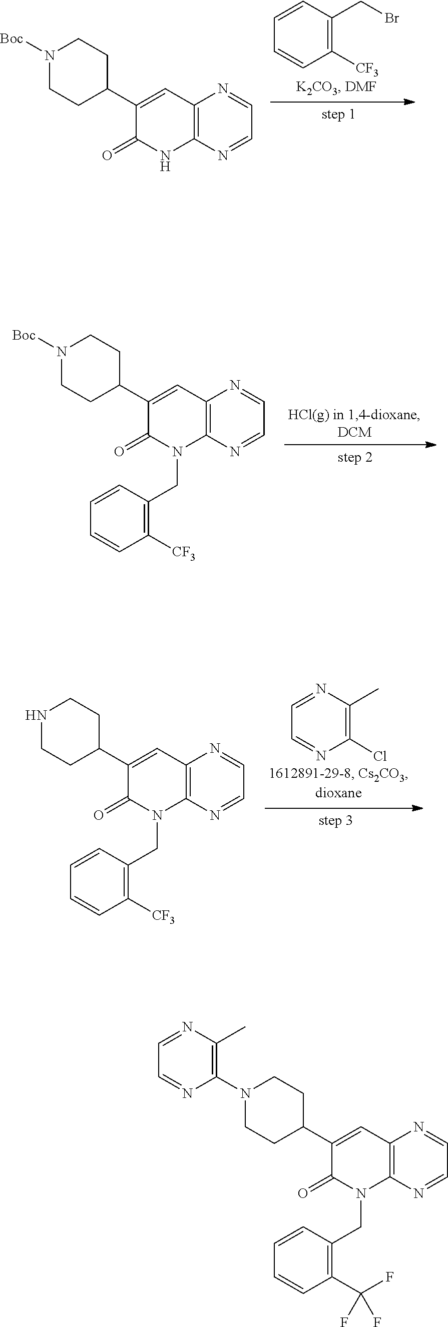

Step 1: tert-butyl 4-(6-oxo-5-(2-(trifluoromethyl)benzyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (210 mg, 0.636 mmol, 1 equiv) and 1-(bromomethyl)-2-(trifluoromethyl)benzene (229 mg, 0.954 mmol, 1.5 equiv) as the starting materials to give tert-butyl 4-(6-oxo-5-(2-(trifluoromethyl)benzyl)-5,6-dihydropyrido[2,3-b]pyrazin-7-yl)piperidine-1-carboxylate (180 mg, 57%) as a white solid. MS m/z: 489 [M+H]⁺.

Step 2: 7-(piperidin-4-yl)-5-(2-(trifluoromethyl)benzyl)pyrido[2,3-b]pyrazin-6(5H)-one: Followed general procedure F using tert-butyl 4-(6-oxo-5-(2-(trifluoromethyl)benzyl)-5,6-dihydropyrido[2,3-b]pyrazin-7-yl)piperidine-1-carboxylate (180 mg, 0.368 mmol, 1 equiv) as the starting material to give the crude product 7-(piperidin-4-yl)-5-(2-(trifluoromethyl)benzyl)pyrido[2,3-b]pyrazin-6(5H)-one (180 mg) as a white solid. MS m/z: 389 [M+H]⁺.

Step 3: 7-(1-(3-methylpyrazin-2-yl)piperidin-4-yl)-5-(2-(trifluoromethyl)benzyl)pyrido[2,3-b]pyrazin-6(5H)-one: Followed general procedure G using 7-(piperidin-4-yl)-5-((3-(trifluoromethyl)pyridin-2-yl)methyl)pyrido[2,3-b]pyrazin-6(5H)-one (65 mg, 0.135 mmol, 1 equiv) and 2-chloro-3-methylpyrazine (26.1 mg, 0.203 mmol, 1.5 equiv) as the starting materials to give 7-(1-(3-methylpyrazin-2-yl)piperidin-4-yl)-5-(2-(trifluoromethyl)benzyl)pyrido[2,3-b]pyrazin-6(5H)-one (62.9 mg, 78.2%) as a white solid. ¹H NMR (400 MHz, Chloroform-d) δ 8.52 (d, 1H), 8.43 (t, 1H), 8.20 (s, 1H), 8.04 (d, 1H), 7.90 (s, 1H), 7.76-7.69 (m, 1H), 7.36-7.30 (m, 2H), 6.66 (d, 1H), 5.95 (s, 2H), 3.82 (d, 2H), 3.29 (t, 1H), 3.09 (t, 2H), 2.66 (s, 3H), 2.17 (d, 2H), 1.90-1.81 (m, 2H). MS m/z: 481.25 [M+H]⁺.

### 7-(1-(3-Methoxypyrazin-2-yl)piperidin-4-yl)-5-(2-(trifluoromethyl)benzyl)pyrido[2,3-b]pyrazin-6(5H)-one (9)

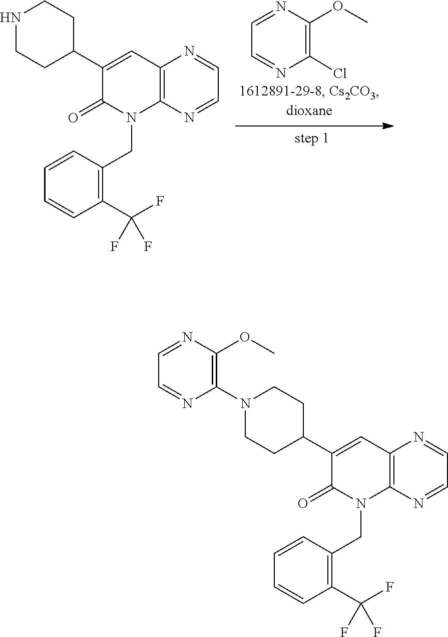

Step 1: 7-(1-(3-methoxypyrazin-2-yl)piperidin-4-yl)-5-(2-(trifluoromethyl)benzyl)pyrido[2,3-b]pyrazin-6(5H)-one: Followed general procedure G using 7-(piperidin-4-yl)-5-(2-(trifluoromethyl)benzyl)pyrido[2,3-b]pyrazin-6(5H)-one (65 mg, 0.135 mmol, 1 equiv) and 2-chloro-3-methoxypyrazine (29.3 mg, 0.203 mmol, 1.5 equiv) as the starting materials to give 7-(1-(3-methoxypyrazin-2-yl)piperidin-4-yl)-5-(2-(trifluoromethyl)benzyl)pyrido[2,3-b]pyrazin-6(5H)-one (49 mg, 72.6%) as a white solid. ¹H NMR (400 MHz, Chloroform-d) δ 8.50 (d, 1H), 8.41 (d, 1H), 7.87-7.85 (m, 1H), 7.76-7.71 (m, 2H), 7.61-7.58 (m, 1H), 7.36-7.30 (m, 2H), 6.68-6.61 (m, 1H), 5.94 (s, 2H), 4.50 (d, 2H), 4.03 (s, 3H), 3.39-3.28 (m, 1H), 3.24-3.07 (m, 2H), 2.17 (d, 2H), 1.94-1.79 (m, 2H). MS m/z: 497.2 [M+H]⁺.

### 5-(2-fluorobenzyl)-7-(1-(2-(trifluoromethyl)phenyl)piperidin-4-yl)pyrido[2,3-b]pyrazin-6(5H)-one (10)

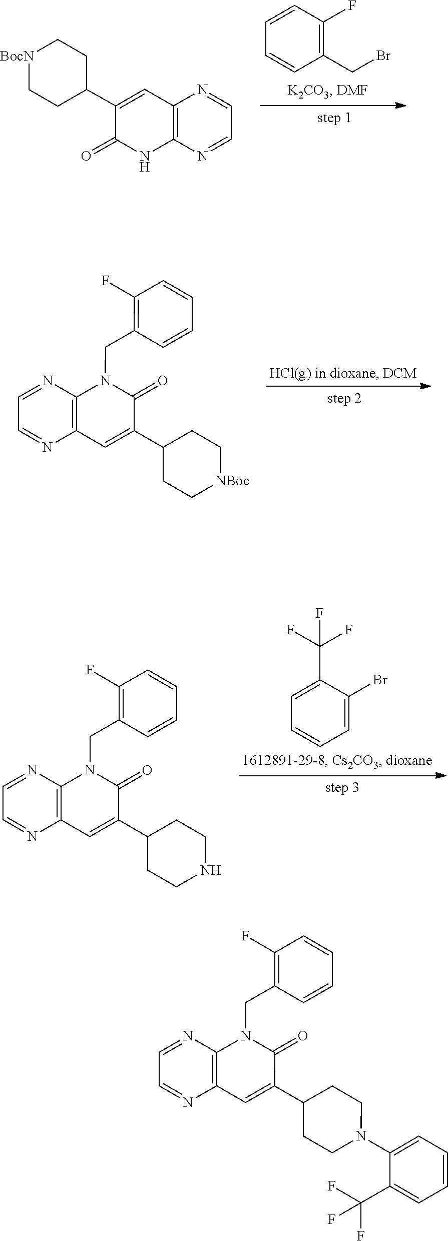 [SMILES: CC(C)(C)OC(=O)N1CCC(c2cc3nccnc3[nH]c2=O)CC1 ; Fc1ccccc1CBr ; CC(C)(C)OC(=O)N1CCC(c2cc3nccnc3n(Cc3ccccc3F)c2=O)CC1 ; O=c1c(C2CCNCC2)cc2nccnc2n1Cc1ccccc1F ; FC(F)(F)c1ccccc1Br ; O=c1c(C2CCN(c3ccccc3C(F)(F)F)CC2)cc2nccnc2n1Cc1ccccc1F]

Step 1: tert-butyl 4-(5-(2-fluorobenzyl)-6-oxo-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (210 mg, 0.636 mmol, 1 equiv) and 1-(bromomethyl)-2-fluorobenzene (180 mg, 0.954 mmol, 1.5 equiv) as the starting materials to give tert-butyl 4-(5-(2-fluorobenzyl)-6-oxo-5,6-dihydropyrido[2,3-b]pyrazin-7-yl)piperidine-1-carboxylate (240 mg, 85.8%) as an off-white solid. MS m/z: 439 [M+H]⁺.

Step 2: 5-(2-fluorobenzyl)-7-(piperidin-4-yl)pyrido[2,3-b]pyrazin-6(5H)-one: Followed general procedure F using tert-butyl 4-(5-(2-fluorobenzyl)-6-oxo-5,6-dihydropyrido[2,3-b]pyrazin-7-yl)piperidine-1-carboxylate (120 mg, 0.274 mmol, 1 equiv) as the starting material to give the crude product 5-(2-fluorobenzyl)-7-(piperidin-4-yl)pyrido[2,3-b]pyrazin-6(5H)-one (100 mg) as an off white solid. MS m/z: 339 [M+H]⁺.

Step 3: 5-(2-fluorobenzyl)-7-(1-(2-(trifluoromethyl)phenyl)piperidin-4-yl)pyrido[2,3-b]pyrazin-6(5H)-one: Followed general procedure G using 5-(2-fluorobenzyl)-7-(piperidin-4-yl)pyrido[2,3-b]pyrazin-6(5H)-one (100 mg, 0.296 mmol, 1 equiv) and 1-bromo-2-(trifluoromethyl)benzene (99.7 mg, 0.444 mmol, 1.5 equiv) as the starting materials to give 5-(2-fluorobenzyl)-7-(1-(2-(trifluoromethyl)phenyl)piperidin-4-yl)pyrido[2,3-b]pyrazin-6(5H)-one (84.1 mg, 58.3%) as a light yellow solid. ¹H NMR (400 MHz, Chloroform-d) δ 8.52-8.49 (m, 1H), 8.47-8.45 (m, 1H), 7.88 (d, 1H), 7.65 (d, 1H), 7.58-7.51 (m, 1H), 7.48 (s, 1H), 7.25-7.18 (m, 2H), 7.10-7.03 (m, 1H), 7.02-6.96 (m, 2H), 5.80 (s, 2H), 3.28-3.13 (m, 3H), 2.98 (t, 2H), 2.12-2.02 (m, 2H), 2.00-1.85 (m, 2H). MS m/z: 483.25 [M+H]⁺.

### 5-(2-Fluorobenzyl)-8-methyl-7-(1-(2-(trifluoromethyl)pyridin-3-yl)piperidin-4-yl)pyrido[2,3-b]pyrazin-6(5H)-one (11)

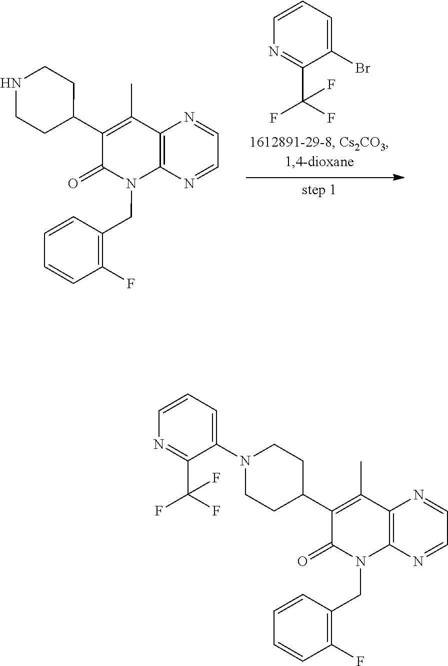 [SMILES: Cc1c(C2CCNCC2)c(=O)n(Cc2ccccc2F)c2nccnc12 ; FC(F)(F)c1ncccc1Br ; Cc1c(C2CCN(c3cccnc3C(F)(F)F)CC2)c(=O)n(Cc2ccccc2F)c2nccnc12]

Step 1: 5-(2-fluorobenzyl)-8-methyl-7-(1-(2-(trifluoromethyl)pyridin-3-yl)piperidin-4-yl)pyrido[2,3-b]pyrazin-6(5H)-one: Followed general procedure G using 5-(2-fluorobenzyl)-8-methyl-7-(piperidin-4-yl)pyrido[2,3-b]pyrazin-6(5H)-one (75 mg, 0.213 mmol, 1 equiv) and 3-bromo-2-(trifluoromethyl)pyridine (57.7 mg, 0.256 mmol, 1.2 equiv) as the starting materials to give 5-(2-fluorobenzyl)-8-methyl-7-(1-(2-(trifluoromethyl)pyridin-3-yl)piperidin-4-yl)pyrido[2,3-b]pyrazine-6(5H)-one (48.6 mg, 44.8%) as an off-white solid. ¹H NMR (400 MHz, DMSO-d₆) δ 8.63 (d, 1H), 8.58 (d, 1H), 8.45-8.43 (m, 1H), 8.03-8.01 (m, 1H), 7.71-7.67 (m, 1H), 7.31-7.20 (m, 2H), 7.05-7.01 (m, 1H), 6.83-6.80 (m, 1H), 5.63 (s, 2H), 3.24 (d, 1H), 3.14 (d, 2H), 2.98-2.93 (m, 2H), 2.69 (s, 3H), 2.65-2.58 (m, 2H), 1.62 (d, 2H). MS m/z: 499.20 [M+H]⁺.

### 7-(1-(2-Fluoro-6-methylphenyl)piperidin-4-yl)-5-((3-(trifluoromethyl)pyrazin-2-yl)methyl)pyrido[2,3-b]pyrazin-6(5H)-one (12)

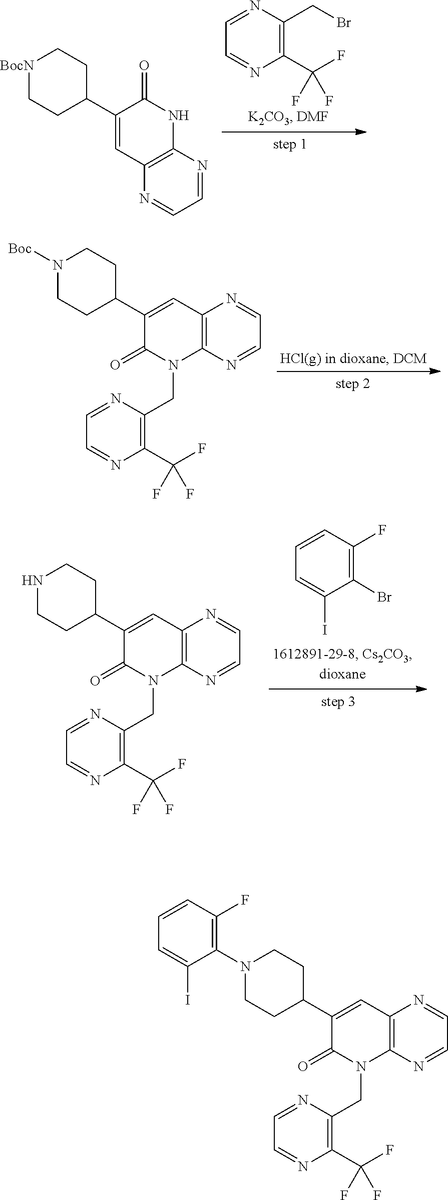 [SMILES: CC(C)(C)OC(=O)N1CCC(c2cc3nccnc3[nH]c2=O)CC1 ; FC(F)(F)c1nccnc1CBr ; CC(C)(C)OC(=O)N1CCC(c2cc3nccnc3n(Cc3nccnc3C(F)(F)F)c2=O)CC1 ; O=c1c(C2CCNCC2)cc2nccnc2n1Cc1nccnc1C(F)(F)F ; Fc1cccc(I)c1Br ; O=c1c(C2CCN(c3c(F)cccc3I)CC2)cc2nccnc2n1Cc1nccnc1C(F)(F)F]

Step 1: tert-butyl 4-(6-OXO-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (45 mg, 0.136 mmol, 1 equiv) and 2-(bromomethyl)-3-(trifluoromethyl)pyrazine (49.2 mg, 0.204 mmol, 1.5 equiv) as the starting materials to give tert-butyl 4-(6-oxo-5-((3-(trifluoromethyl)pyrazin-2-yl)methyl)-5,6-dihydropyrido[2,3-b]pyrazin-7-yl)piperidine-1-carboxylate (30 mg, 44%) as a white solid. MS m/z: 491 [M+H]⁺.

Step 2: 7-(piperidin-4-yl)-5-((3-(trifluoromethyl)pyrazin-2-yl)methyl)pyrido[2,3-b]pyrazin-6(5H)-one: Followed general procedure F using tert-butyl 4-(6-oxo-5-((3-(trifluoromethyl)pyrazin-2-yl)methyl)-5,6-dihydropyrido[2,3-b]pyrazin-7-yl)piperidine-1-carboxylate (30 mg, 0.061 mmol, 1 equiv) as the starting material to give the crude product 7-(piperidin-4-yl)-5-((3-(trifluoromethyl)pyrazin-2-yl)methyl)pyrido[2,3-b]pyrazin-6(5H)-one (20 mg) as a yellow oil. MS m/z: 391 [M+H]⁺.

Step 3: 7-(1-(2-fluoro-6-methylphenyl)piperidin-4-yl)-5-((3-(trifluoromethyl)pyrazin-2-yl)methyl)pyrido[2,3-b]pyrazin-6(5H)-one: Followed general procedure G using 7-(piperidin-4-yl)-5-((3-(trifluoromethyl)pyrazin-2-yl)methyl)pyrido[2,3-b]pyrazin-6(5H)-one (20 mg, 0.041 mmol, 1 equiv) and 2-bromo-1-fluoro-3-methylbenzene (11.5 mg, 0.061 mmol, 1.5 equiv) as the starting materials to give 7-(1-(2-fluoro-6-methylphenyl)piperidin-4-yl)-5-((3-(trifluoromethyl)pyrazin-2-yl)methyl)pyrido[2,3-b]pyrazin-6(5H)-one (7.5 mg, 36.9%) as an off-white solid. ¹H NMR (400 MHz, Chloroform-d) δ 8.53-8.49 (m, 2H), 8.44 (d, 1H), 8.34 (d, 1H), 7.95 (d, 1H), 7.10-6.98 (m, 2H), 6.96-6.87 (m, 1H), 6.09 (s, 2H), 3.54-3.43 (m, 2H), 3.32-3.17 (m, 3H), 2.51 (s, 3H), 2.28-2.14 (m, 2H), 2.10-2.06 (m, 2H). MS m/z: 499.25 [M+H]⁺.

### 7-(1-(2-Fluoro-6-methylphenyl)piperidin-4-yl)-5-((2-(trifluoromethyl)pyridin-3-yl)methyl)pyrido[2,3-b]pyrazin-6(5H)-one (13)

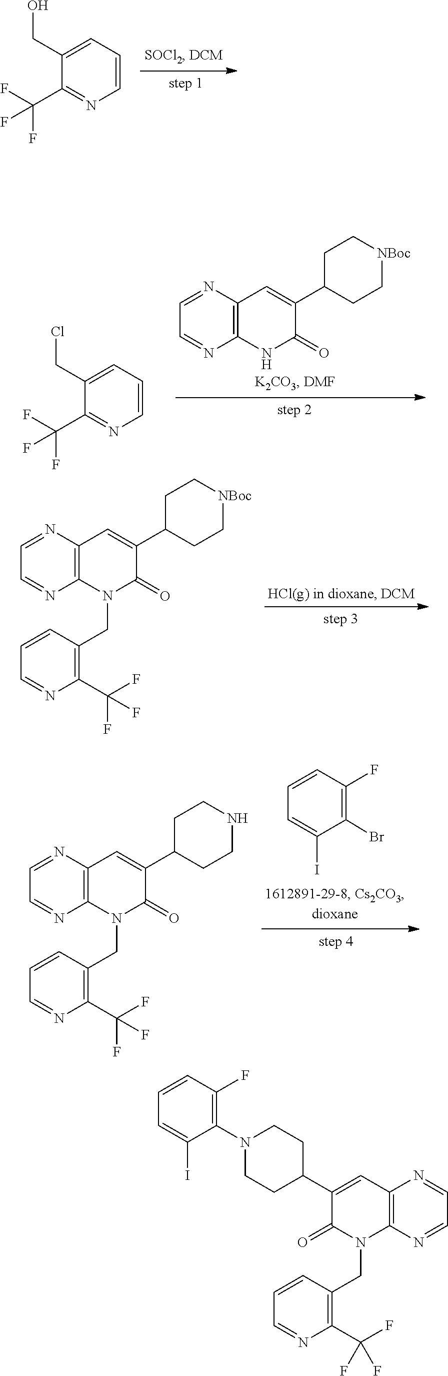

Step 1: 3-(chloromethyl)-2-(trifluoromethyl)pyridine: Followed general procedure J using (2-(trifluoromethyl)pyridin-3-yl)methanol (500 mg, 2.82 mmol, 1.2 equiv) as the starting material to give the crude product 3-(chloromethyl)-2-(trifluoromethyl)pyridine (360 mg) as a yellow solid. MS m/z: 196 [M+H]⁺.

Step 2: tert-butyl 4-(6-oxo-5-((2-(trifluoromethyl)pyridin-3-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (100 mg, 0.302 mmol, 1 equiv) and 3-(chloromethyl)-2-(trifluoromethyl)pyridine (88.8 mg, 0.453 mmol, 1.5 equiv) as the starting materials to give tert-butyl 4-(6-oxo-5-((2-(trifluoromethyl)pyridin-3-yl)methyl)-5,6-dihydropyrido[2,3-b]pyrazin-7-yl)piperidine-1-carboxylate (95 mg, 65%) as a white solid. MS m/z: 490 [M+H]⁺.

Step 3: 7-(piperidin-4-yl)-5-((2-(trifluoromethyl)pyridin-3-yl)methyl)pyrido[2,3-b]pyrazin-6(5H)-one: Followed general procedure F using tert-butyl 4-(6-oxo-5-((2-(trifluoromethyl)pyridin-3-yl)methyl)-5,6-dihydropyrido[2,3-b]pyrazin-7-yl)piperidine-1-carboxylate (70 mg, 0.143 mmol, 1 equiv) as the starting material to give the crude product 7-(piperidin-4-yl)-5-((2-(trifluoromethyl)pyridin-3-yl)methyl)pyrido[2,3-b]pyrazin-6(5H)-one (45 mg) as a yellow oil. MS m/z: 390 [M+H]⁺.

Step 4: 7-(1-(2-fluoro-6-methylphenyl)piperidin-4-yl)-5-((2-(trifluoromethyl)pyridin-3-yl)methyl)pyrido[2,3-b]pyrazin-6(5H)-one: Followed general procedure G using 7-(piperidin-4-yl)-5-((2-(trifluoromethyl)pyridin-3-yl)methyl)pyrido[2,3-b]pyrazin-6(5H)-one (30 mg, 0.077 mmol, 1 equiv) and 2-bromo-1-fluoro-3-methylbenzene (21.8 mg, 0.115 mmol, 1.5 equiv) as the starting materials to give 7-(1-(2-fluoro-6-methylphenyl)piperidin-4-yl)-5-((2-(trifluoromethyl)pyridin-3-yl)methyl)pyrido[2,3-b]pyrazin-6(5H)-one (7.7 mg, 19.9%) as an off-white solid. ¹H NMR (400 MHz, Chloroform-d) δ 8.58 (d, 1H), 8.53 (d, 1H), 8.40 (d, 1H), 8.03-7.92 (m, 1H), 7.33-7.28 (m, 1H), 7.10 (d, 1H), 7.07-6.97 (m, 2H), 6.96-6.86 (m, 1H), 5.96 (s, 2H), 3.53-3.37 (m, 2H), 3.30-3.16 (m, 3H), 2.54-2.38 (m, 3H), 2.19-1.97 (m, 4H). MS m/z: 498.20 [M+H]⁺.

### 7-(1-(2-Fluoro-6-methylphenyl)piperidin-4-yl)-5-((6-(trifluoromethyl)pyridin-3-yl)methyl)pyrido[2,3-b]pyrazin-6(5H)-one (14)

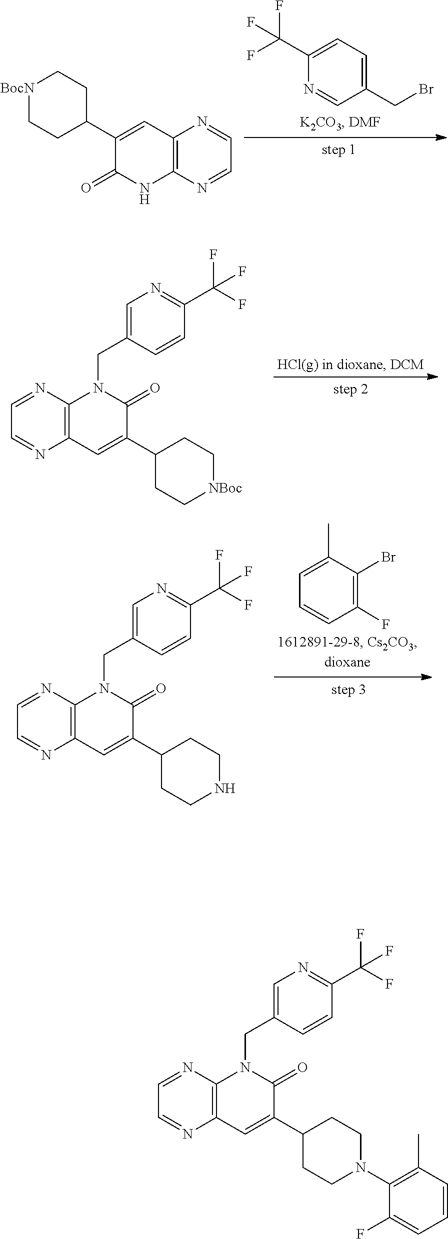

Step 1: tert-butyl 4-(6-oxo-5-((6-(trifluoromethyl)pyridin-3-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (85 mg, 0.257 mmol, 1 equiv) and 5-(bromomethyl)-2-(trifluoromethyl)pyridine (67.9 mg, 0.283 mmol, 1.1 equiv) as the starting materials to give tert-butyl 4-(6-oxo-5-((6-(trifluoromethyl)pyridin-3-yl)methyl)-5,6-dihydropyrido[2,3-b]pyrazin-7-yl)piperidine-1-carboxylate (90 mg, 71%) as an off-white solid. MS m/z 490 [M+H]⁺.

Step 2: 7-(piperidin-4-yl)-5-((6-(trifluoromethyl)pyridin-3-yl)methyl)pyrido[2,3-b]pyrazin-6(5H)-one: Followed general procedure F using tert-butyl 4-(6-oxo-5-((6-(trifluoromethyl)pyridin-3-yl)methyl)-5,6-dihydropyrido[2,3-b]pyrazin-7-yl)piperidine-1-carboxylate (90 mg, 0.184 mmol, 1 equiv) as the starting material to give the crude product 7-(piperidin-4-yl)-5-((6-(trifluoromethyl)pyridin-3-yl)methyl)pyrido[2,3-b]pyrazin-6(5H)-one (60 mg) as a yellow oil. MS m/z 390 [M+H]⁺.

Step 3: 7-(1-(2-fluoro-6-methylphenyl)piperidin-4-yl)-5-((6-(trifluoromethyl)pyridin-3-yl)methyl)pyrido[2,3-b]pyrazin-6(5H)-one: Followed general procedure G using 7-(piperidin-4-yl)-5-((6-(trifluoromethyl)pyridin-3-yl)methyl)pyrido[2,3-b]pyrazin-6(5H)-one (60 mg, 0.154 mmol, 1 equiv) and 2-bromo-1-fluoro-3-methylbenzene (32 mg, 0.169 mmol, 1.1 equiv) as the starting materials to give 7-(1-(2-fluoro-6-methylphenyl)piperidin-4-yl)-5-((6-(trifluoromethyl)pyridin-3-yl)methyl)pyrido[2,3-b]pyrazin-6(5H)-one (37.3 mg, 47.7%) as an off-white solid. ¹H NMR (300 MHz, Chloroform-d) δ 8.96 (s, 1H), 8.59-8.54 (m, 1H), 8.53-8.48 (m, 1H), 8.07 (d, 1H), 7.89 (s, 1H), 7.64 (d, 1H), 7.12-6.88 (m, 3H), 5.81 (s, 2H), 3.56-3.41 (m, 2H), 3.31-3.16 (m, 3H), 2.47 (s, 3H), 2.13-1.94 (m, 4H). MS m/z: 498.25 [M+H]⁺.

### 7-(1-(2-Fluoro-6-methylphenyl)piperidin-4-yl)-5-((5-(trifluoromethyl)pyridin-2-yl)methyl)pyrido[2,3-b]pyrazin-6(5H)-one (15)

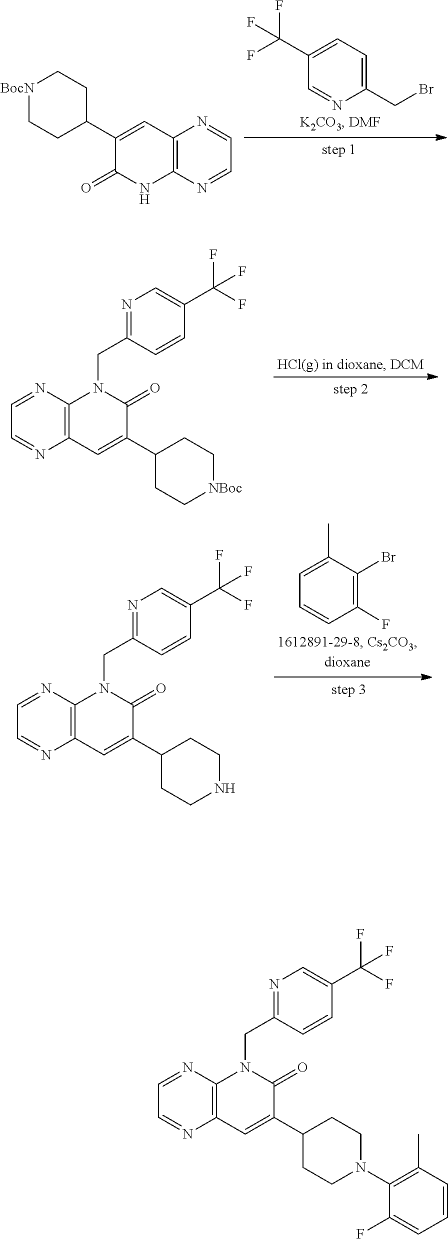

Step 1: tert-butyl 4-(6-oxo-5-((5-(trifluoromethyl)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (85 mg, 0.257 mmol, 1 equiv) and 2-(bromomethyl)-5-(trifluoromethyl)pyridine (67.9 mg, 0.283 mmol, 1.1 equiv) as the starting materials to give tert-butyl 4-(6-oxo-5-((5-(trifluoromethyl)pyridin-2-yl)methyl)-5,6-dihydropyrido[2,3-b]pyrazin-7-yl)piperidine-1-carboxylate (90 mg, 71%) as an off-white solid. MS m/z: 490 [M+H]⁺.

Step 2: 7-(piperidin-4-yl)-5-((5-(trifluoromethyl)pyridin-2-yl)methyl)pyrido[2,3-b]pyrazin-6(5H)-one: Followed general procedure F using tert-butyl 4-(6-oxo-5-(2-(trifluoromethyl)benzyl)-5,6-dihydropyrido[2,3-b]pyrazin-7-yl)piperidine-1-carboxylate (90 mg, 0.184 mmol, 1 equiv) as the starting material to give the crude product 7-(piperidin-4-yl)-5-((5-(trifluoromethyl)pyridin-2-yl)methyl)pyrido[2,3-b]pyrazin-6(5H)-one (60 mg) as a yellow oil. MS m/z 390 [M+H]⁺.

Step 3: 7-(1-(2-fluoro-6-methylphenyl)piperidin-4-yl)-5-((5-(trifluoromethyl)pyridin-2-yl)methyl)pyrido[2,3-b]pyrazin-6(5H)-one: Followed general procedure G using 7-(piperidin-4-yl)-5-((5-(trifluoromethyl)pyridin-2-yl)methyl)pyrido[2,3-b]pyrazin-6(5H)-one (60 mg, 0.154 mmol, 1 equiv) and 2-bromo-1-fluoro-3-methylbenzene (32 mg, 0.169 mmol, 1.1 equiv) as the starting materials to give 7-(1-(2-fluoro-6-methylphenyl)piperidin-4-yl)-5-((5-(trifluoromethyl)pyridin-2-yl)methyl)pyrido[2,3-b]pyrazin-6(5H)-one (28.1 mg, 35.7%) as an off-white solid. ¹H NMR (300 MHz, Chloroform-d) δ 8.76 (s, 1H), 8.57-8.51 (m, 1H), 8.49-8.42 (m, 1H), 7.92 (d, 1H), 7.88 (dd, 1H), 7.37 (d, 1H), 7.07-6.97 (m, 2H), 6.94-6.84 (m, 1H), 5.94 (s, 2H), 3.40 (t, 2H), 3.28-3.09 (m, 3H), 2.42 (s, 3H), 2.14-2.02 (m, 2H), 2.00-1.85 (m, 2H). MS m/z: 498.25 [M+H]⁺.

### 7-(1-(2-Fluoro-6-methylphenyl)piperidin-4-yl)-5-((4-(trifluoromethyl)pyridin-3-yl)methyl) pyrido[2,3-b]pyrazin-6(5H)-one (16)

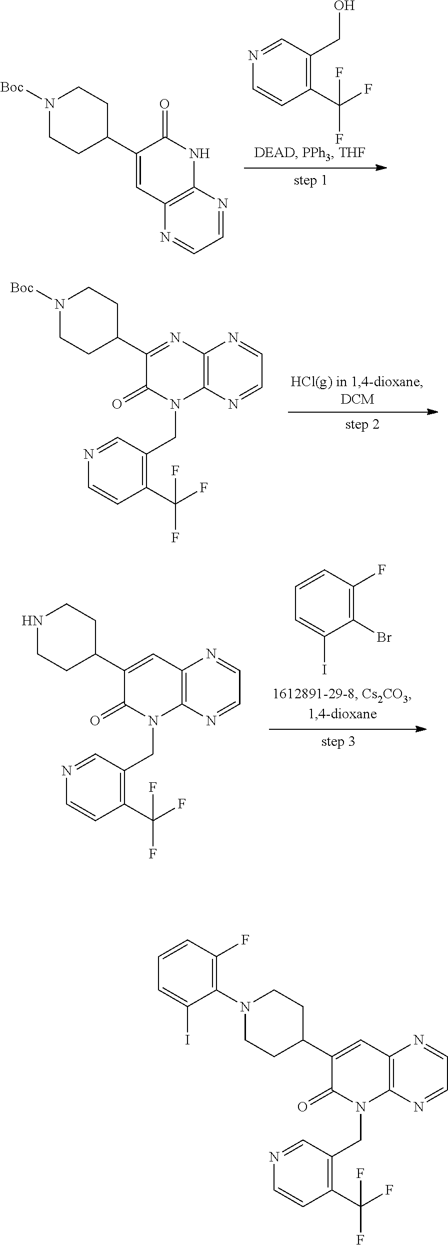 [SMILES: CC(C)(C)OC(=O)N1CCC(c2cc3nccnc3[nH]c2=O)CC1 ; OCc1cnccc1C(F)(F)F ; CC(C)(C)OC(=O)N1CCC(c2nc3nccnc3n(Cc3cnccc3C(F)(F)F)c2=O)CC1 ; O=c1c(C2CCNCC2)cc2nccnc2n1Cc1cnccc1C(F)(F)F ; Fc1cccc(I)c1Br ; O=c1c(C2CCN(c3c(F)cccc3I)CC2)cc2nccnc2n1Cc1cnccc1C(F)(F)F]

Step 1: tert-butyl 4-(6-oxo-5-((4-(trifluoromethyl)pyridin-3-yl)methyl)-5,6-dihydropyrido[2,3-b]pyrazin-7-yl)piperidine-1-carboxylate: Followed general procedure X using tert-butyl 4-(6-oxo-5,6-dihydropyrido[2,3-b]pyrazine-7-yl)piperidine-1-carboxylate (100 mg, 0.303 mmol, 1 equiv) and (4-(trifluoromethyl)pyridin-3-yl)methanol (58.9 mg, 0.303 mmol, 1.1 equiv) as the starting materials to give tert-butyl 4-(6-oxo-5-((4-(trifluoromethyl)pyridin-3-yl)methyl)-5,6-dihydropyrido[2,3-b]pyrazin-7-yl)piperidine-1-carboxylate (87 mg, 58%) as a yellow oil. MS m/z: 490 [M+H]⁺.

Step 2: 7-(piperidin-4-yl)-5-((4-(trifluoromethyl)pyridin-3-yl)methyl)pyrido[2,3-b]pyrazin-6(5H)-one: Followed general procedure F using tert-butyl 4-(6-oxo-5-((4-(trifluoromethyl) pyridin-3-yl)methyl)-5,6-dihydropyrido[2,3-b]pyrazin-7-yl)piperidine-1-carboxylate (80 mg, 0.163 mmol, 1 equiv) as the starting material to give the crude product 7-(piperidin-4-yl)-5-((4-(trifluoromethyl)pyridin-3-yl)methyl)pyrido[2,3-b]pyrazin-6(5H)-one (44.5 mg) as a yellow oil. MS m/z: 390 [M+H]⁺.

Step 3: 7-(1-(2-fluoro-6-methylphenyl)piperidin-4-yl)-5-((4-(trifluoromethyl)pyridin-3-yl)methyl) pyrido[2,3-b]pyrazin-6(5H)-one: Followed general procedure G using 7-(piperidin-4-yl)-5-((4-(trifluoromethyl) pyridin-3-yl)methyl)pyrido[2,3-b]pyrazin-6(5H)-one (35 mg, 0.09 mmol, 1 equiv) and 2-bromo-1-fluoro-3-methylbenzene (20.3 mg, 0.108 mmol, 1.2 equiv) as the starting materials to give 7-(1-(2-fluoro-6-methylphenyl)piperidin-4-yl)-5-((4-(trifluoromethyl)pyridin-3-yl)methyl)pyrido[2,3-b]pyrazin-6(5H)-one (19.9 mg, 41.9%) as an off-white solid. ¹H NMR (400 MHz, Chloroform-d) δ 8.66 (d, 1H), 8.53 (d, 1H), 8.39 (d, 1H), 8.05 (s, 1H), 7.93 (s, 1H), 7.60 (d, 1H), 7.01-6.96 (m, 2H), 6.90-6.84 (m, 1H), 5.96 (s, 2H), 3.39-3.32 (m, 2H), 3.22-3.11 (m, 3H), 2.38 (s, 3H), 2.07-2.00 (m, 2H), 1.90-1.81 (m, 2H). MS m/z: 475.25 [M+H]⁺.

### 7-(1-(2-Fluoro-6-methylphenyl)piperidin-4-yl)-5-((3-(trifluoromethyl)pyridin-4-yl)methyl) pyrido[2,3-b]pyrazin-6(5H)-one (17)

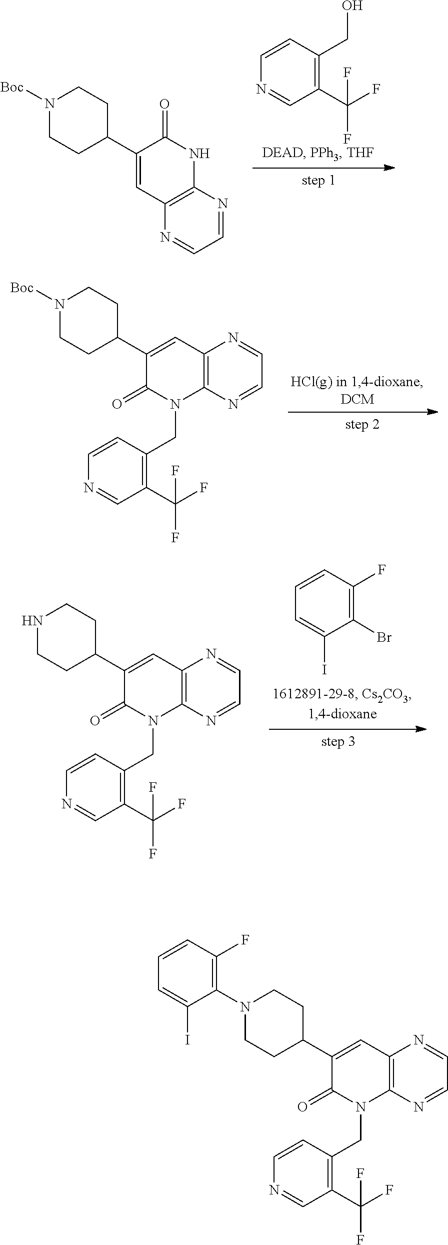 [SMILES: CC(C)(C)OC(=O)N1CCC(c2cc3nccnc3[nH]c2=O)CC1 ; OCc1ccncc1C(F)(F)F ; CC(C)(C)OC(=O)N1CCC(c2cc3nccnc3n(Cc3ccncc3C(F)(F)F)c2=O)CC1 ; O=c1c(C2CCNCC2)cc2nccnc2n1Cc1ccncc1C(F)(F)F ; Fc1cccc(I)c1Br ; O=c1c(C2CCN(c3c(F)cccc3I)CC2)cc2nccnc2n1Cc1ccncc1C(F)(F)F]

Step 1: tert-butyl 4-(6-oxo-5-((3-(trifluoromethyl)pyridin-4-yl)methyl)-5,6-dihydropyrido[2,3-b]pyrazin-7-yl)piperidine-1-carboxylate: Followed general procedure X using tert-butyl 4-(6-oxo-5,6-dihydropyrido[2,3-b]pyrazin-7-yl)piperidine-1-carboxylate (100 mg, 0.303 mmol, 1 equiv) and (3-(trifluoromethyl)pyridin-4-yl)methanol (58.9 mg, 0.303 mmol, 1.1 equiv) as the starting materials to give tert-butyl 4-(6-oxo-5-((3-(trifluoromethyl)pyridin-4-yl)methyl)-5,6-dihydropyrido[2,3-b]pyrazin-7-yl)piperidine-1-carboxylate (50 mg, 33%) as a yellow oil. MS m/z: 490 [M+H]⁺.

Step 2: 7-(piperidin-4-yl)-5-((3-(trifluoromethyl)pyridin-4-yl)methyl)pyrido[2,3-b]pyrazin-6(5H)-one: Followed general procedure F using tert-butyl 4-(6-oxo-5-((3-(trifluoromethyl) pyridin-4-yl)methyl)-5,6-dihydropyrido[2,3-b]pyrazin-7-yl)piperidine-1-carboxylate (50 mg, 0.102 mmol, 1 equiv) as the starting material to give the crude product 7-(piperidin-4-yl)-5-((3-(trifluoromethyl)pyridin-4-yl)methyl)pyrido[2,3-b]pyrazin-6(5H)-one (50 mg) as a yellow oil. MS m/z: 390 [M+H]⁺.

Step 3: 7-(1-(2-fluoro-6-methylphenyl)piperidin-4-yl)-5-((3-(trifluoromethyl)pyridin-4-yl)methyl) pyrido[2,3-b]pyrazin-6(5H)-one: Followed general procedure G using 7-(piperidin-4-yl)-5-((3-(trifluoromethyl) pyridin-4-yl)methyl)pyrido[2,3-b]pyrazin-6(5H)-one (50 mg, 0.128 mmol, 1 equiv) and 2-bromo-1-fluoro-3-methylbenzene (29.1 mg, 0.154 mmol, 1.2 equiv) as the starting materials to give 7-(1-(2-fluoro-6-methylphenyl)piperidin-4-yl)-5-((3-(trifluoromethyl)pyridin-4-yl)methyl) pyrido[2,3-b]pyrazin-6(5H)-one (25.6 mg, 39.6%) as an off-white solid. ¹H NMR (400 MHz, Chloroform-d) δ 8.93 (s, 1H), 8.58-8.55 (m, 2H), 8.39 (d, 1H), 7.98 (s, 1H), 7.05-6.99 (m, 2H), 6.94-6.89 (m, 1H), 6.70 (d, 1H), 5.94 (s, 2H), 3.46 (s, 2H), 3.26-3.19 (m, 3H), 2.48 (s, 3H), 2.08 (s, 4H). MS m/z: 498.20 [M+H]⁺.

### 5-(2-Fluorobenzyl)-7-(1-(2-(trifluoromethyl)pyridin-3-yl)piperidin-4-yl)pyrido[2,3-b]pyrazin-6(5H)-one (18)

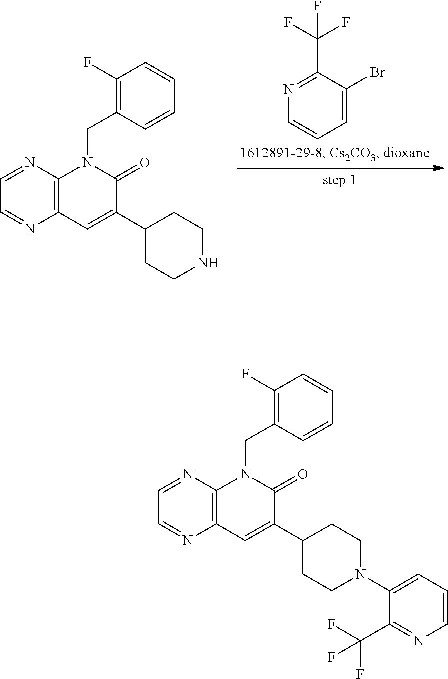 [SMILES: O=c1c(C2CCNCC2)cc2nccnc2n1Cc1ccccc1F ; FC(F)(F)c1ncccc1Br ; O=c1c(C2CCN(c3cccnc3C(F)(F)F)CC2)cc2nccnc2n1Cc1ccccc1F]

Step 1: 5-(2-fluorobenzyl)-7-(1-(2-(trifluoromethyl)pyridin-3-yl)piperidin-4-yl)pyrido[2,3-b]pyrazin-6(5H)-one: Followed general procedure G using 5-(2-fluorobenzyl)-7-(piperidin-4-yl)pyrido[2,3-b]pyrazin-6(5H)-one hydrochloride (100 mg, 0.296 mmol, 1 equiv) and 3-bromo-2-(trifluoromethyl)pyridine (100 mg, 0.444 mmol, 1.5 equiv) as the starting materials to give 5-(2-fluorobenzyl)-7-(1-(2-(trifluoromethyl)pyridin-3-yl)piperidin-4-yl)pyrido[2,3-b]pyrazin-6(5H)-one (69.3 mg, 48.3%) as a white solid. ¹H NMR (400 MHz, Chloroform-d) δ 8.52-8.49 (m, 1H), 8.49-8.44 (m, 2H), 7.88 (s, 1H), 7.75 (d, 1H), 7.51-7.45 (m, 1H), 7.25-7.18 (m, 1H), 7.10-7.03 (m, 1H), 7.02-6.96 (m, 2H), 5.81 (s, 2H), 3.28 (d, 2H), 3.19 (ddd, 1H), 2.97 (t, 2H), 2.16-2.06 (m, 2H), 1.95-1.84 (m, 2H). MS m/z: 484.25 [M+H]⁺.

### 7-(1-(2-Fluoro-6-methylphenyl)piperidin-4-yl)-5-((3-methylpyrazin-2-yl)methyl)pyrido[2,3-b]pyrazin-6(5H)-one (20)

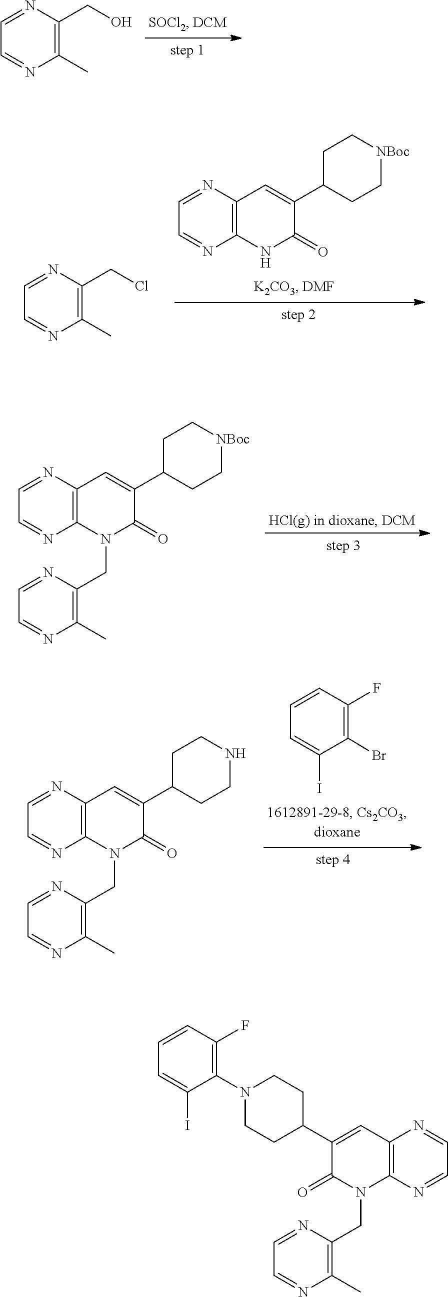

Step 1: 2-(chloromethyl)-3-methylpyrazine: Followed general procedure J using (3-methylpyrazin-2-yl)methanol (200 mg, 1.61 mmol, 1 equiv) and SOCl₂ (574 mg, 4.83 mmol, 3 equiv) as the starting materials to give 2-(chloromethyl)-3-methylpyrazine (170 mg, 74%) as a yellow oil. MS m/z: 143 [M+H]⁺.

Step 2: tert-butyl 4-(5-((3-methylpyrazin-2-yl)methyl)-6-oxo-5,6-dihydropyrido[2,3-b]pyrazin-7-yl)piperidine-1-carboxylate: Followed general procedure E using 2-(chloromethyl)-3-methylpyrazine (200 mg, 1.4 mmol, 1 equiv) and tert-butyl 4-(6-oxo-5,6-dihydropyrido[2,3-b]pyrazin-7-yl)piperidine-1-carboxylate (556 mg, 1.68 mmol, 1.2 equiv) as the starting materials to give tert-butyl 4-(5-((3-methylpyrazin-2-yl)methyl)-6-oxo-5,6-dihydropyrido[2,3-b]pyrazin-7-yl)piperidine-1-carboxylate (320 mg, 52%) as a white solid. MS m/z: 437 [M+H]⁺.

Step 3: 5-((3-methylpyrazin-2-yl)methyl)-7-(piperidin-4-yl)pyrido[2,3-b]pyrazin-6(5H)-one: Followed general procedure F using tert-butyl 4-(5-((3-methylpyrazin-2-yl)methyl)-6-oxo-5,6-dihydropyrido[2,3-b]pyrazin-7-yl)piperidine-1-carboxylate (100 mg, 0.229 mmol, 1 equiv) as the starting material to give the crude product 5-((3-methylpyrazin-2-yl)methyl)-7-(piperidin-4-yl)pyrido[2,3-b]pyrazin-6(5H)-one (65 mg) as a yellow oil. MS m/z: 337 [M+H]⁺.

Step 4: 7-(1-(2-fluoro-6-methylphenyl)piperidin-4-yl)-5-((3-methylpyrazin-2-yl)methyl)pyrido[2,3-b]pyrazin-6(5H)-one: Followed general procedure G using 5-((3-methylpyrazin-2-yl)methyl)-7-(piperidin-4-yl)pyrido[2,3-b]pyrazin-6(5H)-one (40 mg, 0.119 mmol, 1 equiv) and 2-bromo-1-fluoro-3-methylbenzene (33.7 mg, 0.178 mmol, 1.5 equiv) as the starting materials to give 7-(1-(2-fluoro-6-methylphenyl)piperidin-4-yl)-5-((3-methylpyrazin-2-yl)methyl)pyrido[2,3-b]pyrazin-6(5H)-one (13.9 mg, 25.8%) as an off-white solid. ¹H NMR (400 MHz, DMSO-d₆) δ 8.59 (d, 1H), 8.53 (d, 1H), 8.34 (d, 1H), 8.15 (d, 1H), 7.94 (s, 1H), 7.07-6.93 (m, 3H), 5.78 (s, 2H), 3.22-3.15 (m, 2H), 3.10-3.03 (m, 2H), 3.02-2.95 (m, 1H), 2.72 (s, 3H), 2.33 (s, 3H), 1.96-1.89 (m, 2H), 1.87-1.74 (m, 2H). MS m/z: 445.25 [M+H]⁺.

### 7-(1-(2-Fluoro-6-methylphenyl)piperidin-4-yl)-5-((3-(trifluoromethyl)pyridin-2-yl)methyl)pyrido[2,3-b]pyrazin-6(5H)-one (21)

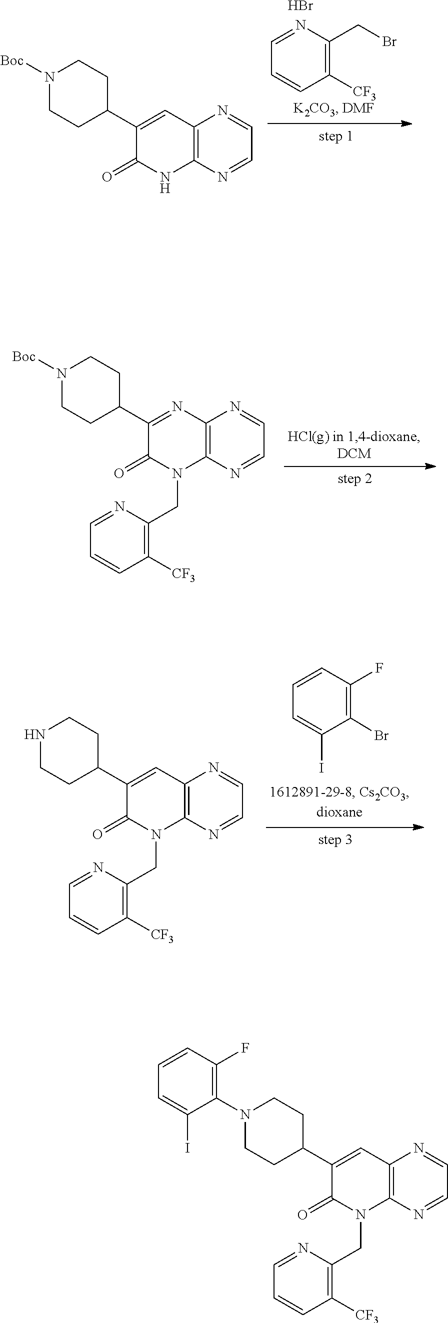

Step 1: tert-butyl 4-(6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (50 mg, 0.151 mmol, 1 equiv) and 2-(bromomethyl)-3-(trifluoromethyl)pyridine hydrogen bromide (48.5 mg, 0.151 mmol, 1 equiv) as the starting materials to give tert-butyl 4-(6-oxo-5-((3-(trifluoromethyl)pyridin-2-yl)methyl)-5,6-dihydropyrido[2,3-b]pyrazin-7-yl)piperidine-1-carboxylate (50 mg, 69%) as a yellow solid. MS m/z: 490 [M+H]⁺.

Step 2: 7-(piperidin-4-yl)-5-((3-(trifluoromethyl)pyridin-2-yl)methyl)pyrido[2,3-b]pyrazin-6(5H)-one: Followed general procedure F using tert-butyl 4-(6-oxo-5-((3-(trifluoromethyl)pyridin-2-yl)methyl)-5,6-dihydropyrido[2,3-b]pyrazin-7-yl)piperidine-1-carboxylate (50 mg, 0.102 mmol, 1 equiv) as the starting material to give the crude product 7-(piperidin-4-yl)-5-((3-(trifluoromethyl)pyridin-2-yl)methyl)pyrido[2,3-b]pyrazin-6(5H)-one (34.6 mg) as a white solid. MS m/z: 390 [M+H]⁺.

Step 3: 7-(1-(2-fluoro-6-methylphenyl)piperidin-4-yl)-5-((3-(trifluoromethyl)pyridin-2-yl)me-thyl)pyrido[2,3-b]pyrazin-6(5H)-one: Followed general procedure G using 7-(piperidin-4-yl)-5-((3-(trifluoromethyl)pyridin-2-yl)methyl)pyrido[2,3-b]pyrazin-6(5H)-one (35 mg, 0.09 mmol, 1 equiv) and 2-bromo-1-fluoro-3-methylbenzene (30.3 mg, 0.135 mmol, 1.5 equiv) as the starting materials to give 7-(1-(2-(trifluoromethyl)phenyl)piperidin-4-yl)-5-((3-(trifluoromethyl)pyridin-2-yl)methyl)pyrido[2,3-b]pyrazin-6(5H)-one (33.2 mg, 42.3%) as a off-white solid. ¹H NMR (400 MHz, Chloroform-d) δ 8.47-8.35 (m, 3H), 7.98-7.95 (m, 1H), 7.91 (d, 1H), 7.25-7.22 (m, 1H), 7.03-6.97 (m, 2H), 6.91-6.86 (m, 1H), 6.04 (s, 2H), 3.42 (d, 2H), 3.23-3.16 (m, 3H), 2.44 (s, 3H), 2.09-1.98 (m, 4H). MS m/z: 498 [M+H]⁺.

### 7-(1-(2-Fluoro-6-methylphenyl)piperidin-4-yl)-8-methyl-5-((3-(trifluoromethyl)pyrazin-2-yl)methyl)pyrido[2,3-b]pyrazin-6(5H)-one (22)

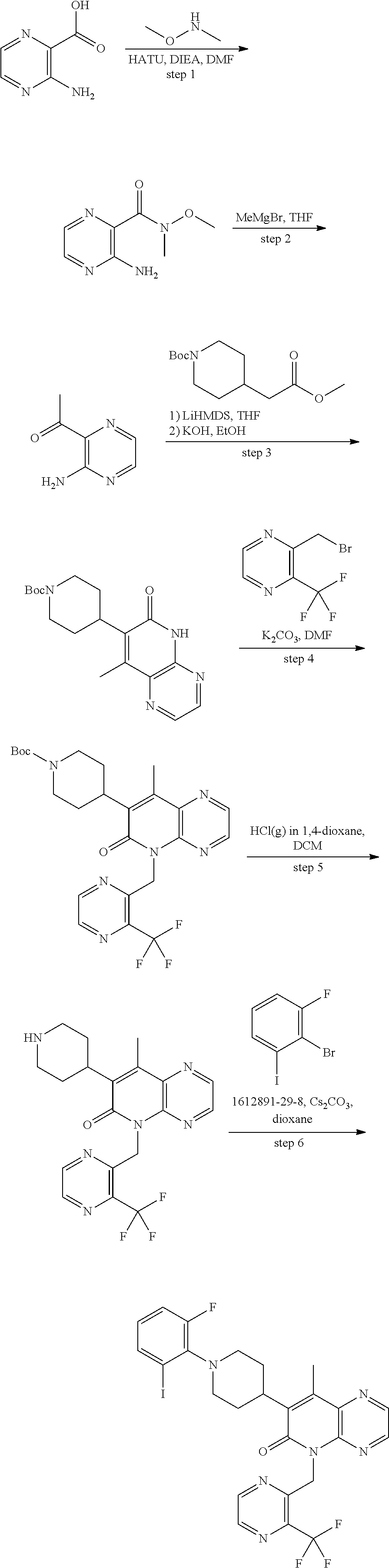

Step 1: 3-amino-N-methoxy-N,5-dimethylpyrazine-2-carboxamide: Followed general procedure A using 3-amino-5-methylpyrazine-2-carboxylic acid (300 mg, 1.95 mmol, 1 equiv) and N,O-dimethylhydroxylamine (179 mg, 2.93 mmol, 1.5 equiv) as the starting materials to give 3-amino-N-methoxy-N,5-dimethylpyrazine-2-carboxamide (320 mg, 83%) as an off-white solid. MS m/z: 183 [M+H]⁺.

Step 2: 1-(3-aminopyrazin-2-yl)ethan-1-one: Followed general procedure C using 3-amino-N-methoxy-N-methylpyrazine-2-carboxamide (500 mg, 2.74 mmol, 1 equiv) as the starting material to give the crude product 1-(3-aminopyrazin-2-yl)ethan-1-one (400 mg) as an off-white solid. MS m/z: 138 [M+H]⁺.

Step 3: tert-butyl 4-(8-methyl-6-oxo-5,6-dihydropyrido[2,3-b]pyrazin-7-yl)piperidine-1-carboxylate: Followed general procedure D using 1-(3-aminopyrazin-2-yl)ethan-1-one (200 mg, 1.45 mmol, 1 equiv) and tert-butyl 4-(2-methoxy-2-oxoethyl)piperidine-1-carboxylate (450 mg, 1.75 mmol, 1.2 equiv) as the starting materials to give tert-butyl 4-(8-methyl-6-oxo-5,6-dihydropyrido[2,3-b]pyrazin-7-yl)piperidine-1-carboxylate (200 mg, 39%) as an off-white solid. MS m/z: 345 [M+H]⁺.

Step 4: tert-butyl 4-(8-methyl-6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (70 mg, 0.203 mmol, 1 equiv) and 2-(bromomethyl)-3-(trifluoromethyl)pyrazine (53.8 mg, 0.223 mmol, 1.1 equiv) as the starting materials to give tert-butyl 4-(8-methyl-6-oxo-5-((3-(trifluoromethyl)pyrazin-2-yl)methyl)-5,6-dihydropyrido[2,3-b]pyrazin-7-yl)piperidine-1-carboxylate (50 mg, 48%) as an off-white solid. MS m/z: 505 [M+H]⁺.

Step 5: 8-methyl-7-(piperidin-4-yl)-5-((3-(trifluoromethyl)pyrazin-2-yl)methyl)pyrido[2,3-b]pyrazin-6(5H)-one: Followed general procedure F using tert-butyl 4-(8-methyl-6-oxo-5-((3-(trifluoromethyl)pyrazin-2-yl)methyl)-5,6-dihydropyrido[2,3-b]pyrazin-7-yl)piperidine-1-carboxylate (50 mg, 0.099 mmol, 1 equiv) as the starting material to give the crude product 8-methyl-7-(piperidin-4-yl)-5-((3-(trifluoromethyl)pyrazin-2-yl)methyl)pyrido[2,3-b]pyrazin-6(5H)-one (40 mg) as a light yellow oil. MS m/z: 405 [M+H]⁺.

Step 6: 7-(1-(2-fluoro-6-methylphenyl)piperidin-4-yl)-8-methyl-5-((3-(trifluoromethyl)pyrazin-2-yl)methyl)pyrido[2,3-b]pyrazin-6(5H)-one: Followed general procedure G using 8-methyl-7-(piperidin-4-yl)-5-((3-(trifluoromethyl)pyrazin-2-yl)methyl)pyrido[2,3-b]pyrazin-6(5H)-one (40 mg, 0.099 mmol, 1 equiv) and 2-bromo-1-fluoro-3-methylbenzene (28 mg, 0.149 mmol, 1.5 equiv) as the starting materials to give 7-(1-(2-fluoro-6-methylphenyl)piperidin-4-yl)-8-methyl-5-((3-(trifluoromethyl)pyrazin-2-yl)methyl)pyrido[2,3-b]pyrazin-6(5H)-one (21.3 mg, 42%) as an off-white solid. ¹H NMR (400 MHz, Chloroform-d) δ 8.52-8.46 (m, 2H), 8.46-8.41 (m, 1H), 8.28 (d, 1H), 7.03-6.93 (m, 2H), 6.92-6.81 (m, 1H), 6.07 (s, 2H), 3.35-3.24 (m, 3H), 3.15-3.08 (m, 2H), 2.78 (s, 3H), 2.75-2.64 (m, 2H), 2.39 (s, 3H), 1.68-1.61 (m, 2H). MS m/z: 513.25 [M+H]⁺.

### 7-(1-(2-Fluoro-6-methylphenyl)pyrrolidin-3-yl)-5-((3-(trifluoromethyl)pyrazin-2-yl)methyl) pyrido[2,3-b]pyrazin-6(5H)-one (23)

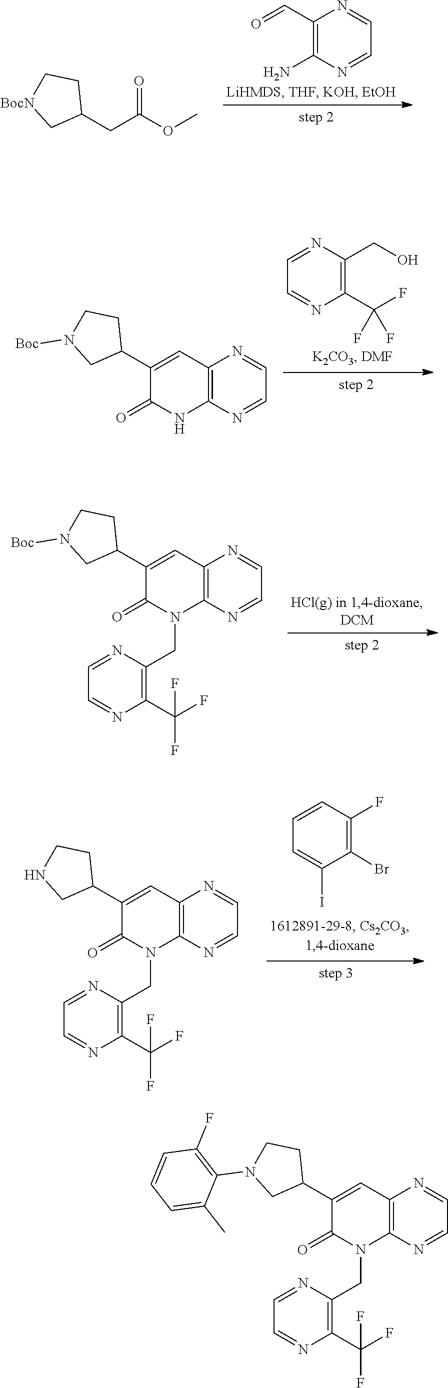

Step 1: tert-butyl 3-(6-oxo-5,6-dihydropyrido[2,3-b]pyrazin-7-yl)pyrrolidine-1-carboxylate: Followed general procedure D using 3-aminopyrazine-2-carbaldehyde (120 mg, 0.975 mmol, 1 equiv) and tert-butyl 3-(2-methoxy-2-oxoethyl)pyrrolidine-1-carboxylate (284 mg, 1.17 mmol, 1.2 equiv) as the starting materials to give tert-butyl 3-(6-oxo-5,6-dihydropyrido[2,3-b]pyrazin-7-yl)pyrrolidine-1-carboxylate (191 mg, 62%) as a light yellow solid. MS m/z: 317 [M+H]⁺.

Step 2: tert-butyl 3-(6-oxo-5-((3-(trifluoromethyl)pyrazin-2-yl)methyl)-5,6-dihydropyrido[2,3-b]pyrazine-7-yl)pyrrolidine-1-carboxylate: Followed general procedure E using tert-butyl 3-(6-oxo-5,6-dihydropyrido[2,3-b]pyrazin-7-yl)pyrrolidine-1-carboxylate (80 mg, 0.253 mmol, 1 equiv) and 2-(bromomethyl)-3-(trifluoromethyl)pyrazine (73.1 mg, 0.304 mmol, 1.2 equiv) as the starting material to give tert-butyl 3-(6-oxo-5-((3-(trifluoromethyl)pyrazin-2-yl)methyl)-5,6-dihydropyrido[2,3-b]pyrazin-7-yl)pyrrolidine-1-carboxylate (60 mg, 49%) as a light yellow oil. MS m/z: 477 [M+H]⁺.

Step 3: 7-(pyrrolidin-3-yl)-5-((3-(trifluoromethyl)pyrazin-2-yl)methyl)pyrido[2,3-b]pyrazin-6(5H)-one: Followed general procedure F using tert-butyl 3-(6-oxo-5-((3-(trifluoromethyl) pyrazine-2-yl)methyl)-5,6-dihydropyrido[2,3-b]pyrazin-7-yl)pyrrolidine-1-carboxylate (60 mg, 0.126 mmol, 1 equiv) as the starting material to give the crude product 7-(pyrrolidin-3-yl)-5-((3-(trifluoromethyl)pyrazin-2-yl)methyl)pyrido[2,3-b]pyrazin-6(5H)-one (30 mg) as a light yellow oil. MS m/z: 377 [M+H]⁺.

Step 4: 7-(1-(2-fluoro-6-methylphenyl)pyrrolidin-3-yl)-5-((3-(trifluoromethyl)pyrazin-2-yl) methyl)pyrido[2,3-b]pyrazin-6(5H)-one: Followed general procedure G using 7-(pyrrolidin-3-yl)-5-((3-(trifluoromethyl) pyrazin-2-yl)methyl)pyrido[2,3-b]pyrazin-6(5H)-one (30 mg, 0.08 mmol, 1 equiv) and 2-bromo-1-fluoro-3-methylbenzene (18 mg, 0.096 mmol, 1.2 equiv) as the starting materials to give 7-(1-(2-fluoro-6-methylphenyl)pyrrolidin-3-yl)-5-((3-(trifluoromethyl)pyrazin-2-yl)methyl)pyrido[2,3-b]pyrazin-6(5H)-one (9.3 mg, 23.7%) as a yellow semi-solid. ¹H NMR (400 MHz, Chloroform-d) δ 8.51-8.49 (m, 2H), 8.43 (d, 1H), 8.33 (d, 1H), 8.10 (s, 1H), 6.97-6.90 (m, 3H), 6.08 (s, 2H), 3.84 (d, 2H), 3.36 (d, 2H), 2.45 (d, 4H), 2.08 (d, 1H), 1.25 (s, 1H). MS m/z: 485.20 [M+H]⁺.

### 7-(4-(2-Fluoro-6-methylphenyl)piperazin-1-yl)-5-((3-(trifluoromethyl)pyrazin-2-yl)methyl)pyrido[2,3-b]pyrazin-6(5H)-one (24)

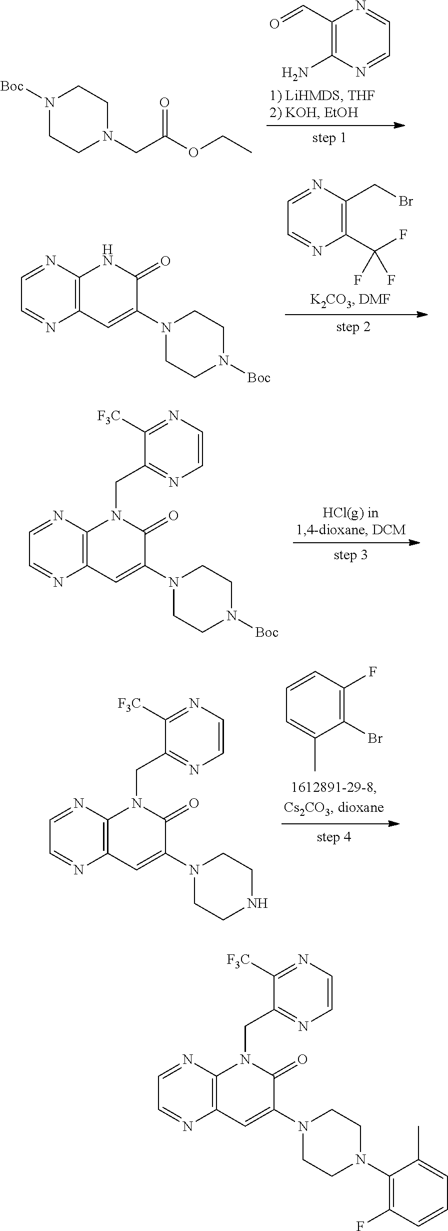

Step 1: tert-butyl 4-(6-oxo-5,6-dihydropyrido[2,3-b]pyrazin-7-yl)piperazine-1-carboxylate: Followed general procedure D using tert-butyl 4-(2-ethoxy-2-oxoethyl)piperazine-1-carboxylate (150 mg, 0.551 mmol, 1 equiv) and 3-aminopyrazine-2-carbaldehyde (81.3 mg, 0.661 mmol, 1.2 equiv) as the starting materials to give tert-butyl 4-(6-oxo-5,6-dihydropyrido[2,3-b]pyrazin-7-yl)piperazine-1-carboxylate (60 mg, 32%) as a yellow solid. MS m/z: 332 [M+H]⁺.

Step 2: tert-butyl 4-(6-oxo-5-((3-(trifluoromethyl)pyrazin-2-yl)methyl)-5,6-dihydropyrido[2,3-b]pyrazin-7-yl)piperazine-1-carboxylate: Followed general procedure E using tert-butyl 4-(6-oxo-5,6-dihydropyrido[2,3-b]pyrazin-7-yl)piperazine-1-carboxylate (60 mg, 0.181 mmol, 1 equiv) and 2-(bromomethyl)-3-(trifluoromethyl)pyrazine (65.4 mg, 0.271 mmol, 1.5 equiv) as the starting materials to give tert-butyl 4-(6-oxo-5-((3-(trifluoromethyl)pyrazin-2-yl)methyl)-5,6-dihydropyrido[2,3-b]pyrazin-7-yl)piperazine-1-carboxylate (45 mg, 50%) as a yellow solid. MS m/z: 492 [M+H]⁺.

Step 3: 7-(piperazin-1-yl)-5-((3-(trifluoromethyl)pyrazin-2-yl)methyl)pyrido[2,3-b]pyrazin-6(5H)-one: Followed general procedure F using tert-butyl 4-(6-oxo-5-((3-(trifluoromethyl)pyrazin-2-yl)methyl)-5,6-dihydropyrido[2,3-b]pyrazin-7-yl)piperazine-1-carboxylate (45 mg, 0.092 mmol, 1 equiv) as the starting material to give the crude product 7-(piperazin-1-yl)-5-((3-(trifluoromethyl)pyrazin-2-yl)methyl)pyrido[2,3-b]pyrazin-6(5H)-one (35 mg) as a yellow oil. MS m/z: 392 [M+H]⁺.

Step 4: 7-(4-(2-fluoro-6-methylphenyl)piperazin-1-yl)-5-((3-(trifluoromethyl)pyrazin-2-yl)methyl)pyrido[2,3-b]pyrazin-6(5H)-one: Followed general procedure G using 7-(piperazin-1-yl)-5-((3-(trifluoromethyl)pyrazin-2-yl)methyl)pyrido[2,3-b]pyrazin-6(5H)-one (35 mg, 0.089 mmol, 1 equiv) and 2-bromo-1-fluoro-3-methylbenzene (25.2 mg, 0.134 mmol, 1.5 equiv) as the starting materials to give 7-(4-(2-fluoro-6-methylphenyl)piperazin-1-yl)-5-((3-(trifluoromethyl)pyrazin-2-yl)methyl)pyrido[2,3-b]pyrazin-6(5H)-one (17.5 mg, 39.1%) as an orange solid. ¹H NMR (400 MHz, Chloroform-d) δ 8.51 (s, 1H), 8.45 (s, 1H), 8.35 (d, 1H), 8.19 (d, 1H), 7.32 (s, 1H), 7.07-6.93 (m, 2H), 6.92-6.81 (m, 1H), 6.09 (s, 2H), 4.11-3.43 (m, 4H), 3.43-3.14 (m, 4H), 2.38 (s, 3H). MS m/z: 500.2 [M+H]⁺.

### 7-(1-(2-Fluoro-6-methylphenyl)piperidin-4-yl)-3-methyl-5-((3-(trifluoromethyl)pyrazin-2-yl)methyl)pyrido[2,3-b]pyrazin-6(5H)-one (25)

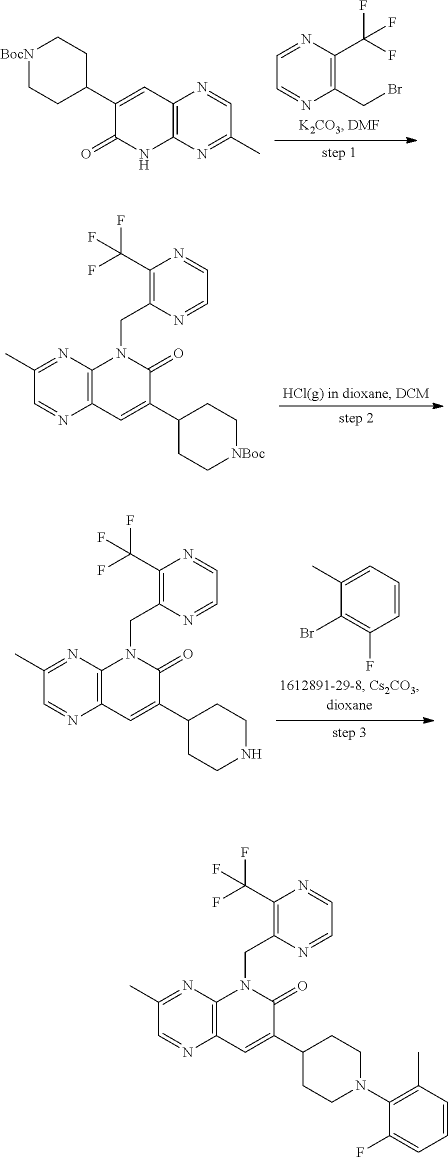

Step 1: tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.290 mmol, 1 equiv) and 2-(bromomethyl)-3-(trifluoromethyl)pyrazine (104 mg, 0.435 mmol, 1.5 equiv) as the starting materials to give tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (90 mg, 61%) as an off-white solid. MS m/z: 505 [M+H]⁺.

Step 2: 3-methyl-7-(piperidin-4-yl)-5-((3-(trifluoromethyl)pyrazin-2-yl)methyl)pyrido[2,3-b]pyrazin-6(5H)-one: Followed general procedure F using tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (90 mg, 0.178 mmol, 1 equiv) as the starting material to give the crude product 3-methyl-7-(piperidin-4-yl)-5-((3-(trifluoromethyl)pyrazin-2-yl)methyl)pyrido[2,3-b]pyrazin-6(5H)-one (60 mg) as a yellow oil. MS m/z: 405 [M+H]⁺.

Step 3: 7-(1-(5-fluoro-2-methylpyridin-3-yl)piperidin-4-yl)-3-methyl-5-((3-(trifluoromethyl)pyrazin-2-yl)methyl)pyrido[2,3-b]pyrazin-6(5H)-one: Followed general procedure G using 3-methyl-7-(piperidin-4-yl)-5-((3-(trifluoromethyl)pyrazin-2-yl)methyl)pyrido[2,3-b]pyrazin-6(5H)-one (60 mg, 0.148 mmol, 1 equiv) and 2-bromo-1-fluoro-3-methylbenzene (42 mg, 0.222 mmol, 1.5 equiv) as the starting materials to give 7-(1-(2-fluoro-6-methylphenyl)piperidin-4-yl)-3-methyl-5-((3-(trifluoromethyl)pyrazin-2-yl)methyl)pyrido[2,3-b]pyrazin-6(5H)-one (21.6 mg, 27.3%) as an off-white solid. ¹H NMR (300 MHz, Chloroform-d) δ 8.55-8.44 (m, 2H), 8.37 (s, 1H), 7.97 (s, 1H), 7.21-7.10 (m, 1H), 7.08-6.89 (m, 2H), 6.08 (s, 2H), 3.64 (t, 2H), 3.51-3.40 (m, 2H), 3.34-3.17 (m, 1H), 2.67 (s, 3H), 2.52 (s, 5H), 2.15-2.08 (m, 2H). MS m/z: 513.2 [M+H]⁺.

### 7-(1-(2-fluoro-5-methylpyridin-4-yl)piperidin-4-yl)-5-(2-(trifluoromethyl)benzyl)pyrido-[2,3-b]pyrazin-6(5H)-one (26)

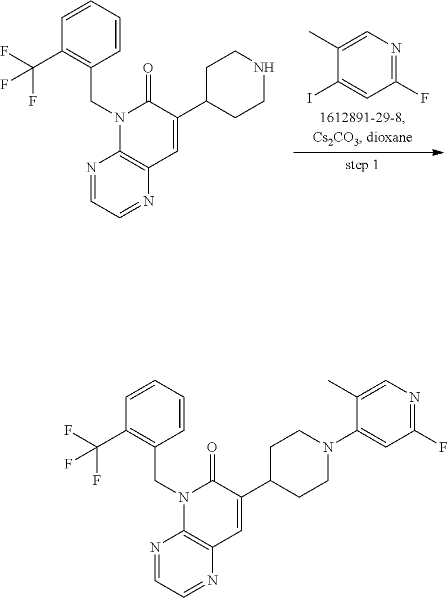

Step 1: 7-(1-(2-fluoro-5-methylpyridin-4-yl)piperidin-4-yl)-5-(2-(trifluoromethyl)benzyl)pyrido-[2,3-b]pyrazin-6(5H)-one: Followed general procedure G using 7-(piperidin-4-yl)-5-(2-(trifluoromethyl)benzyl)pyrido[2,3-b]pyrazin-6(5H)-one (60 mg, 0.154 mmol, 1 equiv) and 2-fluoro-4-iodo-5-methylpyridine (50 mg, 0.231 mmol, 1.5 equiv) as the starting materials to give 7-(1-(2-fluoro-5-methylpyridin-4-yl)piperidin-4-yl)-5-(2-(trifluoromethyl)benzyl)pyrido[2,3-b]pyrazin-6(5H)-one (55 mg, 69.6%) as an off-white solid. ¹H NMR (400 MHz, Chloroform-d) δ 8.52 (d, 1H), 8.43 (d, 1H), 7.88 (dd, 2H), 7.78-7.70 (m, 1H), 7.32 (td, 2H), 6.69-6.62 (m, 1H), 6.44 (s, 1H), 5.95 (s, 2H), 3.58-3.50 (m, 2H), 3.25 (m, 1H), 2.93 (m, 2H), 2.20-2.15 (m, 2H), 1.94-1.79 (m, 2H). MS m/z: 498.20 [M+H]⁺.

### 7-(1-(2-Fluoro-6-methylphenyl)piperidin-4-yl)-5-((5-(trifluoromethyl)pyrazin-2-yl)methyl)pyrido[2,3-b]pyrazin-6(5H)-one (27)

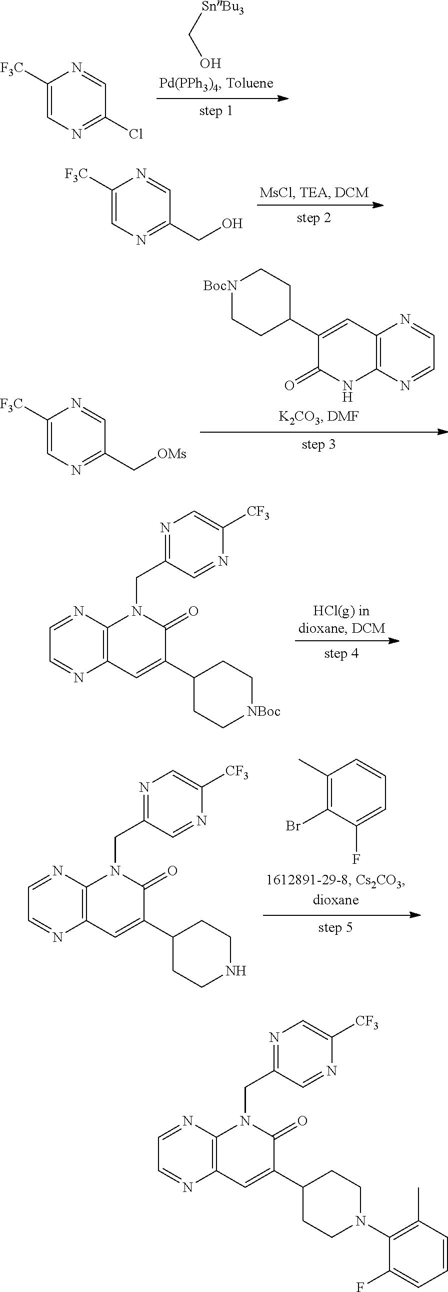

Step 1: (5-(trifluoromethyl)pyrazin-2-yl)methanol: Followed general procedure V using 2-chloro-5-(trifluoromethyl)pyrazine (500 mg, 2.73 mmol, 1 equiv) and (tributylstannyl)methanol (1055 mg, 3.28 mmol, 1.2 equiv) as the starting materials to give (5-(trifluoromethyl)pyrazin-2-yl)methanol (120 mg, 24%) as a white oil. MS m/z: 179 [M+H]⁺.

Step 2: (5-(trifluoromethyl)pyrazin-2-yl)methyl methanesulfonate: Followed general procedure W using (5-(trifluoromethyl)pyrazin-2-yl)methanol (100 mg, 0.561 mmol, 1 equiv) and MsCl (96.4 mg, 0.842 mmol, 1.5 equiv) as the starting materials to (5-(trifluoromethyl)pyrazin-2-yl)methyl methanesulfonate (80 mg, 55%) as a yellow oil. MS m/z: 257 [M+H]⁺.

Step 3: tert-butyl 4-(6-oxo-5-((5-(trifluoromethyl)pyrazin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (77.3 mg, 0.234 mmol, 1.2 equiv) and (5-(trifluoromethyl)pyrazin-2-yl)methyl methanesulfonate (50 mg, 0.195 mmol, 1 equiv) as the starting materials to give tert-butyl 4-(6-oxo-5-((5-(trifluoromethyl)pyrazin-2-yl)methyl)-5,6-dihydropyrido[2,3-b]pyrazin-7-yl)piperidine-1-carboxylate (70 mg, 73%) as an off-white solid. MS m/z: 490 [M+H]⁺.

Step 4: 7-(piperidin-4-yl)-5-((5-(trifluoromethyl)pyrazin-2-yl)methyl)pyrido[2,3-b]pyrazin-6(5H)-one: Followed general procedure F using tert-butyl 4-(6-oxo-5-((5-(trifluoromethyl)pyrazin-2-yl)methyl)-5,6-dihydropyrido[2,3-b]pyrazin-7-yl)piperidine-1-carboxylate (70 mg, 0.143 mmol, 1 equiv) as the starting material to give the crude product 7-(piperidin-4-yl)-5-((5-(trifluoromethyl)pyrazin-2-yl)methyl)pyrido[2,3-b]pyrazin-6(5H)-one (40 mg) as a yellow oil. MS m/z: 390 [M+H]⁺.

Step 5: 7-(1-(2-fluoro-6-methylphenyl)piperidin-4-yl)-5-((5-(trifluoromethyl)pyrazin-2-yl)methyl)pyrido[2,3-b]pyrazin-6(5H)-one: Followed general procedure G using 7-(piperidin-4-yl)-5-((5-(trifluoromethyl)pyrazin-2-yl)methyl)pyrido[2,3-b]pyrazin-6(5H)-one (40 mg, 0.102 mmol, 1 equiv) and 2-bromo-1-fluoro-3-methylbenzene (29 mg, 0.153 mmol, 1.5 equiv) as the starting materials to give 7-(1-(2-fluoro-6-methylphenyl)piperidin-4-yl)-5-((5-(trifluoromethyl)pyrazin-2-yl)methyl)pyrido[2,3-b]pyrazin-6(5H)-one (13.6 mg, 26.6%) as a yellow solid. ¹H NMR (400 MHz, Chloroform-d) δ 8.81-8.72 (m, 2H), 8.53 (d, 1H), 8.41 (d, 1H), 7.91 (d, 1H), 7.07-6.97 (m, 2H), 6.94-6.85 (m, 1H), 5.98 (s, 2H), 3.48-3.32 (m, 2H), 3.28-3.07 (m, 3H), 2.41 (s, 3H), 2.09-2.02 (m, 2H), 1.99-1.87 (m, 2H). MS m/z: 499.20 [M+H]⁺.

### 6-(1-(2-fluoro-6-methylphenyl)piperidin-4-yl)-8-((3-(trifluoromethyl)pyrazin-2-yl)methyl)pyrido[2,3-d]pyrimidin-7(8H)-one (28)

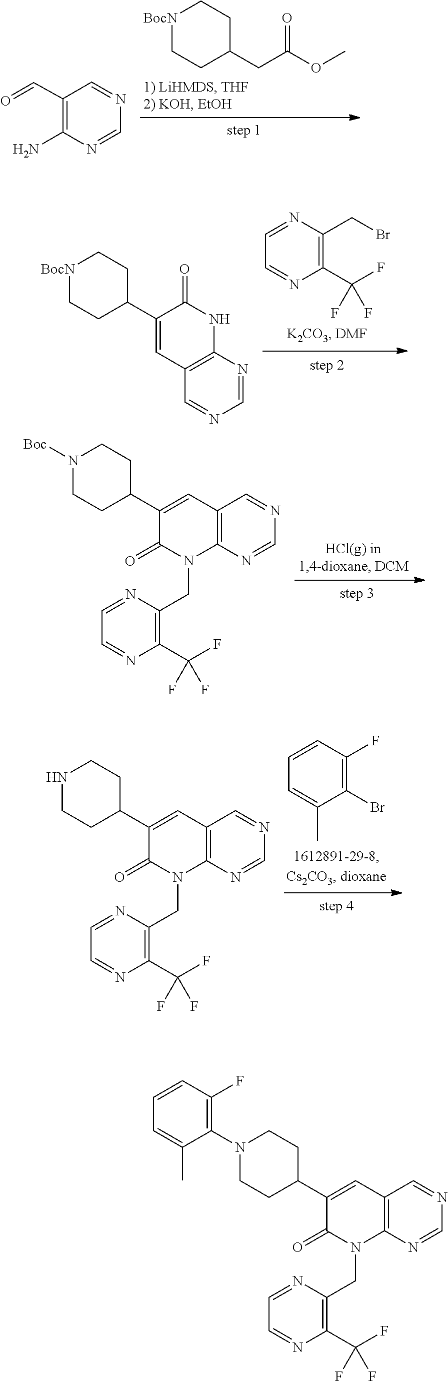

Step 1: tert-butyl 4-(7-oxo-7,8-dihydropyrido[2,3-d]pyrimidin-6-yl)piperidine-1-carboxylate: Followed general procedure D using tert-butyl 4-(2-methoxy-2-oxoethyl)piperidine-1-carboxylate (752 mg, 2.92 mmol, 1.2 equiv) and 4-aminopyrimidine-5-carbaldehyde (300 mg, 2.43 mmol, 1 equiv) as the starting materials to give tert-butyl 4-(7-oxo-7,8-dihydropyrido[2,3-d]pyrimidin-6-yl)piperidine-1-carboxylate (90 mg, 11%) as a yellow solid. MS m/z: 331 [M+H]⁺.

Step 2: tert-butyl 4-(7-oxo-8-((3-(trifluoromethyl)pyrazin-2-yl)methyl)-7,8-dihydropyrido[2,3-d]pyrimidin-6-yl)piperidine-1-carboxylate: Followed general procedure E using tert-butyl 4-(7-oxo-7,8-dihydropyrido[2,3-d]pyrimidin-6-yl)piperidine-1-carboxylate (60 mg, 0.182 mmol, 1 equiv) and 2-(bromomethyl)-3-(trifluoromethyl)pyrazine (43.7 mg, 0.182 mmol, 1 equiv) as the starting materials to give tert-butyl 4-(7-oxo-8-((3-(trifluoromethyl)pyrazin-2-yl)methyl)-7,8-dihydropyrido[2,3-d]pyrimidin-6-yl)piperidine-1-carboxylate (50 mg, 56%) as a white solid. MS m/z: 491 [M+H]⁺.

Step 3: 6-(piperidin-4-yl)-8-((3-(trifluoromethyl)pyrazin-2-yl)methyl)pyrido[2,3-d]pyrimidin-7(8H)-one: Followed general procedure F using tert-butyl 4-(7-oxo-8-((3-(trifluoromethyl)pyrazin-2-yl)methyl)-7,8-dihydropyrido[2,3-d]pyrimidin-6-yl)piperidine-1-carboxylate (45 mg, 0.102 mmol, 1 equiv) as the starting material to give the crude product 6-(piperidin-4-yl)-8-((3-(trifluoromethyl)pyrazin-2-yl)methyl)pyrido[2,3-d]pyrimidin-7(8H)-one (30 mg) as a yellow oil. MS m/z: 391 [M+H]⁺.

Step 4: 6-(1-(2-fluoro-6-methylphenyl)piperidin-4-yl)-8-((3-(trifluoromethyl)pyrazin-2-yl)methyl)pyrido[2,3-d]pyrimidin-7(8H)-one: Followed general procedure G using 6-(piperidin-4-yl)-8-((3-(trifluoromethyl)pyrazin-2-yl)methyl)pyrido[2,3-d]pyrimidin-7(8H)-one (30 mg, 0.091 mmol, 1 equiv) and 2-bromo-1-fluoro-3-methylbenzene (32.8 mg, 0.137 mmol, 1.5 equiv) as the starting materials to give 6-(1-(2-fluoro-6-methylphenyl)piperidin-4-yl)-8-((3-(trifluoromethyl)pyrazin-2-yl)methyl)pyrido[2,3-d]pyrimidin-7(8H)-one (6 mg, 13.4%) as a light yellow solid. ¹H NMR (400 MHz, Chloroform-d) δ 8.81 (s, 1H), 8.70 (s, 1H), 8.55-8.50 (m, 1H), 8.43-8.39 (m, 1H), 7.90 (s, 1H), 7.01-6.94 (m, 2H), 6.92-6.81 (m, 1H), 5.97 (s, 2H), 3.41-3.27 (m, 2H), 3.21-3.06 (m, 3H), 2.36 (s, 3H), 2.08-1.98 (m, 2H), 1.87-1.76 (m, 2H). MS m/z: 499.20 [M+H]⁺.

### 7-(1-(2-Fluoro-3-methylpyridin-4-yl)piperidin-4-yl)-5-(2-(trifluoromethyl)benzyl)pyrido-[2,3-b]pyrazin-6(5H)-one (29)

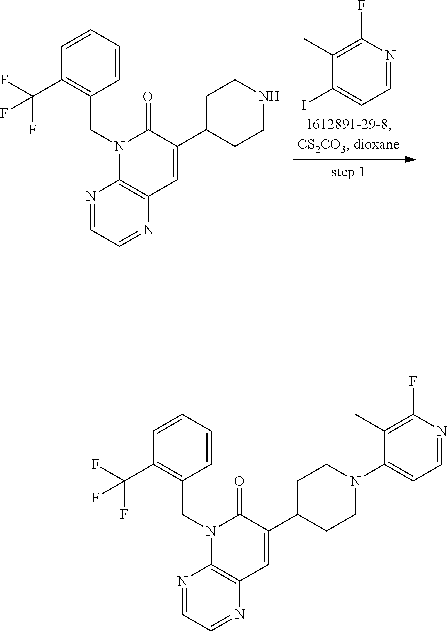

Step 1: 7-(1-(2-fluoro-3-methylpyridin-4-yl)piperidin-4-yl)-5-(2-(trifluoromethyl)benzyl)-pyrido[2,3-b]pyrazin-6(5H)-one: Followed general procedure G using 7-(piperidin-4-yl)-5-(2-(trifluoromethyl)benzyl)pyrido[2,3-b]pyrazin-6(5H)-one (40 mg, 0.103 mmol, 1 equiv) and 2-fluoro-4-iodo-3-methylpyridine (26.8 mg, 0.113 mmol, 1.1 equiv) as the starting materials to give 7-(1-(2-fluoro-3-methylpyridin-4-yl)piperidin-4-yl)-5-(2-(trifluoromethyl)benzyl)pyrido[2,3-b]pyrazin-6(5H)-one (21.3 mg, 41.5%) as a white solid. ¹H NMR (400 MHz, Chloroform-d) δ 8.52 (d, 1H), 8.43 (d, 1H), 7.94 (d, 1H), 7.91-7.88 (m, 1H), 7.77-7.68 (m, 1H), 7.35-7.30 (m, 2H), 6.79 (d, 1H), 6.68-6.62 (m, 1H), 5.95 (s, 2H), 3.55 (d, 2H), 3.31-3.20 (m, 1H), 3.01 (t, 2H), 2.25-2.20 (m, 3H), 2.18 (d, 2H), 1.92-1.83 (m, 2H). MS m/z: 498.20 [M+H]⁺.

### 7-(1-(2-Fluoro-6-methylphenyl)piperidin-4-yl)-5-((6-(trifluoromethyl)pyrazin-2-yl)methyl)pyrido[2,3-b]pyrazin-6(5H)-one (30)

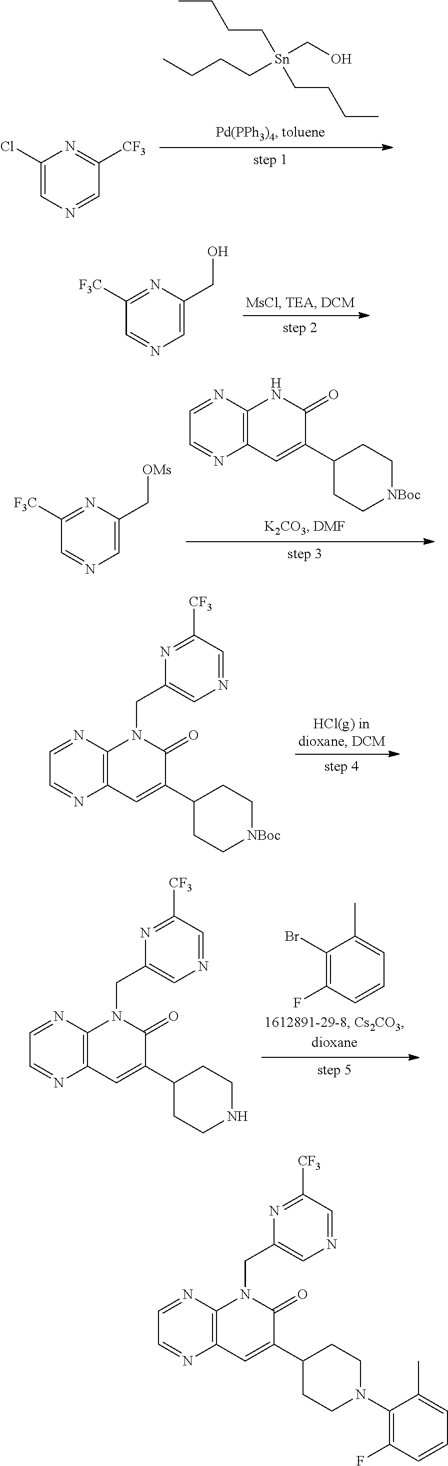

Step 1: (6-(trifluoromethyl)pyrazin-2-yl)methanol: Followed general procedure V using 2-chloro-6-(trifluoromethyl)pyrazine (400 mg, 2.19 mmol, 1 equiv) and (tributylstannyl)methanol (774 mg, 2.41 mmol, 1.1 equiv) as the starting materials to give (6-(trifluoromethyl)pyrazin-2-yl)methanol (140 mg, 35%) as a colorless oil MS m/z: 179 [M+H]⁺.

Step 2: (6-(trifluoromethyl)pyrazin-2-yl)methyl methanesulfonate: Followed general procedure W using (6-(trifluoromethyl)pyrazin-2-yl)methanol (140 mg, 0.786 mmol, 1 equiv) and MsCl (135 mg, 1.17 mmol, 1.5 equiv) as the starting materials to give (6-(trifluoromethyl)pyrazin-2-yl)methyl methanesulfonate (60 mg, 29%) as a colorless oil. MS m/z: 257 [M+H]⁺.

Step 3: tert-butyl 4-(6-oxo-5-((6-(trifluoromethyl)pyrazin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (77.3 mg, 0.234 mmol, 1 equiv) and (6-(trifluoromethyl)pyrazin-2-yl)methyl methanesulfonate (60 mg, 0.234 mmol, 1 equiv) as the starting materials to give tert-butyl 4-(6-oxo-5-((6-(trifluoromethyl)pyrazin-2-yl)methyl)-5,6-dihydropyrido[2,3-b]pyrazin-7-yl)piperidine-1-carboxylate (50 mg, 43%) as a white solid. MS m/z: 491 [M+H]⁺.

Step 4: 7-(piperidin-4-yl)-5-((6-(trifluoromethyl)pyrazin-2-yl)methyl)pyrido[2,3-b]pyrazin-6(5H)-one: Followed general procedure F using tert-butyl 4-(6-oxo-5-((6-(trifluoromethyl)pyrazin-2-yl)methyl)-5,6-dihydropyrido[2,3-b]pyrazin-7-yl)piperidine-1-carboxylate (50 mg, 0.102 mmol, 1 equiv) as the starting material to give the crude product 7-(piperidin-4-yl)-5-((6-(trifluoromethyl)pyrazin-2-yl)methyl)pyrido[2,3-b]pyrazin-6(5H)-one (40 mg) as a yellow oil. MS m/z: 391 [M+H]⁺.

Step 5: 7-(1-(2-fluoro-6-methylphenyl)piperidin-4-yl)-5-((6-(trifluoromethyl)pyrazin-2-yl)methyl)pyrido[2,3-b]pyrazin-6(5H)-one: Followed general procedure G using 7-(piperidin-4-yl)-5-((6-(trifluoromethyl)pyrazin-2-yl)methyl)pyrido[2,3-b]pyrazin-6(5H)-one (40 mg, 0.102 mmol, 1 equiv) and 2-bromo-1-fluoro-3-methylbenzene (29 mg, 0.153 mmol, 1.5 equiv) as the starting materials to give 7-(1-(2-fluoro-6-methylphenyl)piperidin-4-yl)-5-((6-(trifluoromethyl)pyrazin-2-yl)methyl)pyrido[2,3-b]pyrazin-6(5H)-one (19.8 mg, 37.7%) as an off-white solid. ¹H NMR (400 MHz, Chloroform-d) δ 8.81 (s, 1H), 8.71 (s, 1H), 8.56-8.50 (m, 1H), 8.43-8.37 (m, 1H), 7.92 (s, 1H), 7.07-6.95 (m, 2H), 6.94-6.84 (m, 1H), 5.98 (s, 2H), 3.46-3.36 (m, 2H), 3.22-3.14 (m, 3H), 2.43 (s, 3H), 2.09-1.96 (m, 4H). MS m/z: 499.25 [M+H]⁺.

### 7-(1-(2-Fluoro-4-methylpyridin-3-yl)piperidin-4-yl)-5-(2-(trifluoromethyl)benzyl)pyrido[2,3-b]pyrazin-6(5H)-one (31)

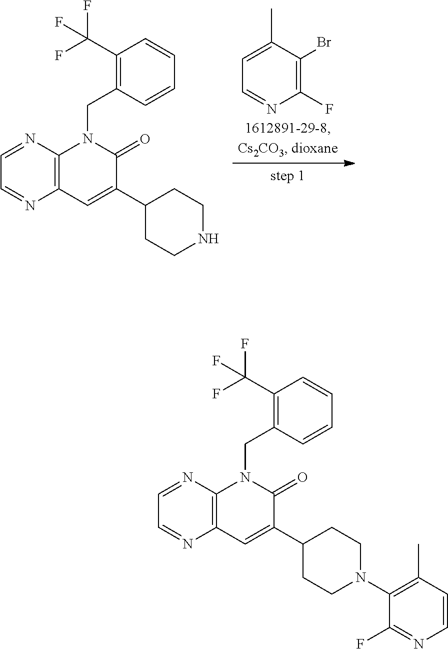

Step 1: 7-(1-(2-fluoro-4-methylpyridin-3-yl)piperidin-4-yl)-5-(2-(trifluoromethyl)benzyl)pyrido[2,3-b]pyrazin-6(5H)-one: Followed general procedure G using 7-(piperidin-4-yl)-5-(2-(trifluoromethyl)benzyl)pyrido[2,3-b]pyrazin-6(5H)-one (60 mg, 0.154 mmol, 1 equiv) and 3-bromo-2-fluoro-4-methylpyridine (44 mg, 0.231 mmol, 1.5 equiv) as the starting materials to give 7-(1-(2-fluoro-4-methylpyridin-3-yl)piperidin-4-yl)-5-(2-(trifluoromethyl)benzyl)pyrido[2,3-b]pyrazin-6(5H)-one (20 mg, 24.7%) as an off-white solid. ¹H NMR (400 MHz, Chloroform-d) δ 8.53-8.49 (m, 1H), 8.44-8.40 (m, 1H), 7.90 (d, 1H), 7.82 (dd, 1H), 7.75-7.70 (m, 1H), 7.37-7.29 (m, 2H), 7.01 (d, 1H), 6.73-6.62 (m, 1H), 5.95 (s, 2H), 3.35-3.26 (m, 2H), 3.25-3.16 (m, 1H), 3.09-2.99 (m, 2H), 2.40 (s, 3H), 2.14-2.04 (m, 2H), 1.91-1.74 (m, 2H). MS m/z: 498.25 [M+H]⁺.

### 7-(1-(6-Fluoro-4-methylpyridin-3-yl)piperidin-4-yl)-5-(2-(trifluoromethyl)benzyl)pyrido-[2,3-b]pyrazin-6(5H)-one (32)

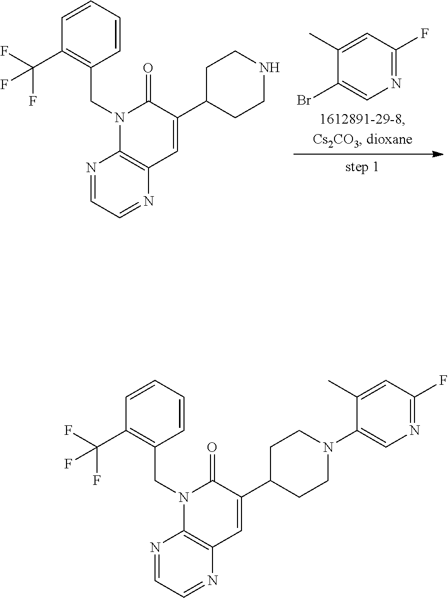

Step 1: 7-(1-(6-fluoro-4-methylpyridin-3-yl)piperidin-4-yl)-5-(2-(trifluoromethyl)benzyl)-pyrido[2,3-b]pyrazin-6(5H)-one: Followed general procedure G using 7-(piperidin-4-yl)-5-(2-(trifluoromethyl)benzyl)pyrido[2,3-b]pyrazin-6(5H)-one (50 mg, 0.129 mmol, 1.00 equiv) and 5-bromo-2-fluoro-4-methylpyridine (26.9 mg, 0.142 mmol, 1.1 equiv) as the starting materials to give 7-(1-(6-fluoro-4-methylpyridin-3-yl)piperidin-4-yl)-5-(2-(trifluoromethyl)benzyl)pyrido[2,3-b]pyrazin-6(5H)-one (30.6 mg, 47.1%) as a white solid. ¹H NMR (300 MHz, Chloroform-d) δ 8.51 (d, 1H), 8.42 (d, 1H), 7.89 (d, 2H), 7.77-7.68 (m, 1H), 7.39-7.29 (m, 2H), 6.75 (d, 1H), 6.70-6.60 (m, 1H), 5.95 (s, 2H), 3.33-3.13 (m, 3H), 3.01-2.88 (m, 2H), 2.38 (s, 3H), 2.14 (d, 2H), 1.98-1.81 (m, 2H). MS m/z: 498.30 [M+H]⁺.

### 7-(1-(2-Fluoro-4-methoxypyridin-3-yl)piperidin-4-yl)-5-(2-(trifluoromethyl)benzyl)pyrido[2,3-b]pyrazin-6(5H)-one (33)

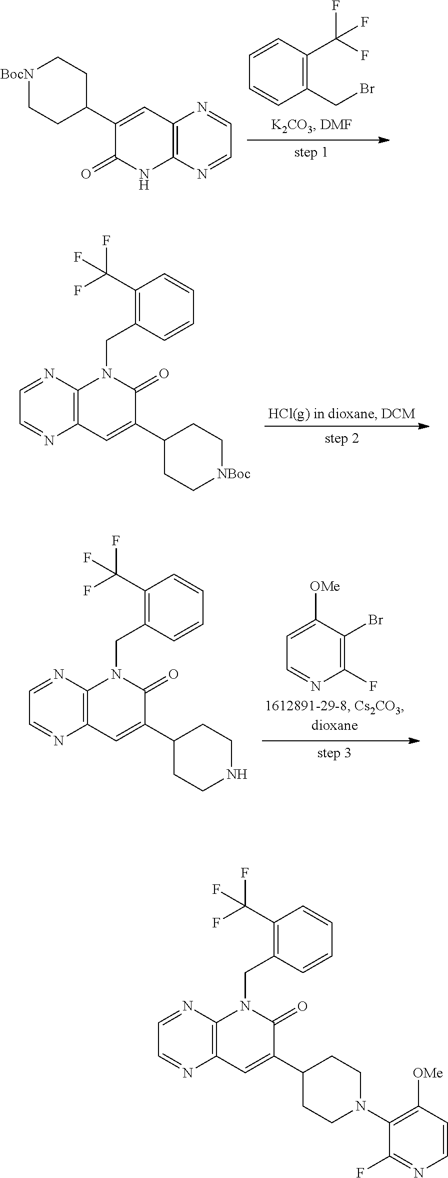

Step 1: tert-butyl 4-(6-oxo-5-(2-(trifluoromethyl)benzyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (200 mg, 0.605 mmol, 1 equiv) and 1-(bromomethyl)-2-(trifluoromethyl)benzene (217 mg, 0.907 mmol, 1.5 equiv) as the starting materials to give tert-butyl 4-(6-oxo-5-(2-(trifluoromethyl)benzyl)-5,6-dihydropyrido[2,3-b]pyrazin-7-yl)piperidine-1-carboxylate (250 mg, 84%) as an off-white solid. MS m/z: 489 [M+H]⁺.

Step 2: 7-(piperidin-4-yl)-5-(2-(trifluoromethyl)benzyl)pyrido[2,3-b]pyrazin-6(5H)-one: Followed general procedure F using tert-butyl 4-(6-oxo-5-(2-(trifluoromethyl)benzyl)-5,6-dihydropyrido[2,3-b]pyrazin-7-yl)piperidine-1-carboxylate (250 mg, 0.512 mmol, 1 equiv) as the starting material to give the crude product 7-(piperidin-4-yl)-5-(2-(trifluoromethyl)benzyl)pyrido[2,3-b]pyrazin-6(5H)-one (190 mg) as a yellow oil. MS m/z: 389 [M+H]⁺.

Step 3: 7-(1-(2-fluoro-4-methoxypyridin-3-yl)piperidin-4-yl)-5-(2-(trifluoromethyl)benzyl)pyrido[2,3-b]pyrazin-6(5H)-one: Followed general procedure G using 7-(piperidin-4-yl)-5-(2-(trifluoromethyl)benzyl)pyrido[2,3-b]pyrazin-6(5H)-one (50 mg, 0.129 mmol, 1 equiv) and 3-bromo-2-fluoro-4-methoxypyridine (39.7 mg, 0.194 mmol, 1.5 equiv) as the starting materials to give 7-(1-(2-fluoro-4-methoxypyridin-3-yl)piperidin-4-yl)-5-(2-(trifluoromethyl)benzyl)pyrido[2,3-b]pyrazin-6(5H)-one (9.7 mg, 14.2%) as an off-white solid. ¹H NMR (300 MHz, Chloroform-d) δ 8.53-8.38 (m, 2H), 7.90 (d, 1H), 7.82 (dd, 1H), 7.77-7.69 (m, 1H), 7.37-7.29 (m, 2H), 6.69 (dd, 2H), 5.94 (s, 2H), 3.93 (s, 3H), 3.37-3.12 (m, 5H), 2.10-1.97 (m, 2H), 1.94-1.77 (m, 2H). MS m/z: 514.3 [M+H]⁺.

### 7-(1-(3-Fluoropyrazin-2-yl)piperidin-4-yl)-5-(2-(trifluoromethyl)benzyl)pyrido[2,3-b]pyrazine-6(5H)-one (34)

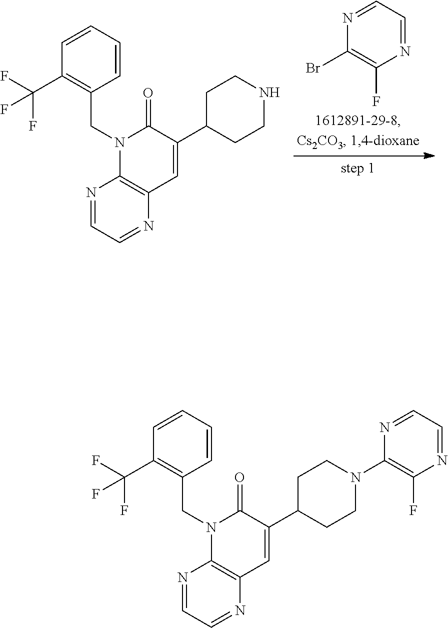 [SMILES: O=c1c(C2CCNCC2)cc2nccnc2n1Cc1ccccc1C(F)(F)F ; Fc1nccnc1Br ; O=c1c(C2CCN(c3nccnc3F)CC2)cc2nccnc2n1Cc1ccccc1C(F)(F)F]

Step 1: 7-(1-(3-fluoropyrazin-2-yl)piperidin-4-yl)-5-(2-(trifluoromethyl)benzyl)pyrido[2,3-b]pyrazine-6(5H)-one: Followed general procedure G using 7-(piperidin-4-yl)-5-(2-(trifluoromethyl)benzyl) pyrido[2,3-b]pyrazin-6(5H)-one (110 mg, 0.283 mmol, 1 equiv) and 2-bromo-3-fluoropyrazine (55.1 mg, 0.311 mmol, 1.1 equiv) as the starting materials to give 7-(1-(3-fluoropyrazin-2-yl)piperidin-4-yl)-5-(2-(trifluoromethyl)benzyl)pyrido[2,3-b]pyrazine-6(5H)-one (10.8 mg, 7.7%) as a white solid. ¹H NMR (400 MHz, Chloroform-d) δ 8.51-8.50 (m, 1H), 8.42-8.41 (m, 1H), 8.00-7.99 (m, 1H), 7.84 (s, 1H), 7.74-7.71 (m, 1H), 7.52-7.50 (m, 1H), 7.35-7.30 (m, 2H), 6.66-6.63 (m, 1H), 5.94 (s, 2H), 4.42 (d, 2H), 3.36-3.28 (m, 1H), 3.10 (t, 2H), 2.13 (d, 2H), 1.83-1.72 (m, 2H). MS m/z: 485.20 [M+H]⁺.

### 7-(1-(2-Fluoro-6-methylphenyl)piperidin-4-yl)-5-(2-fluorobenzyl)pyrido[2,3-b]pyrazin-6(5H)-one (35)

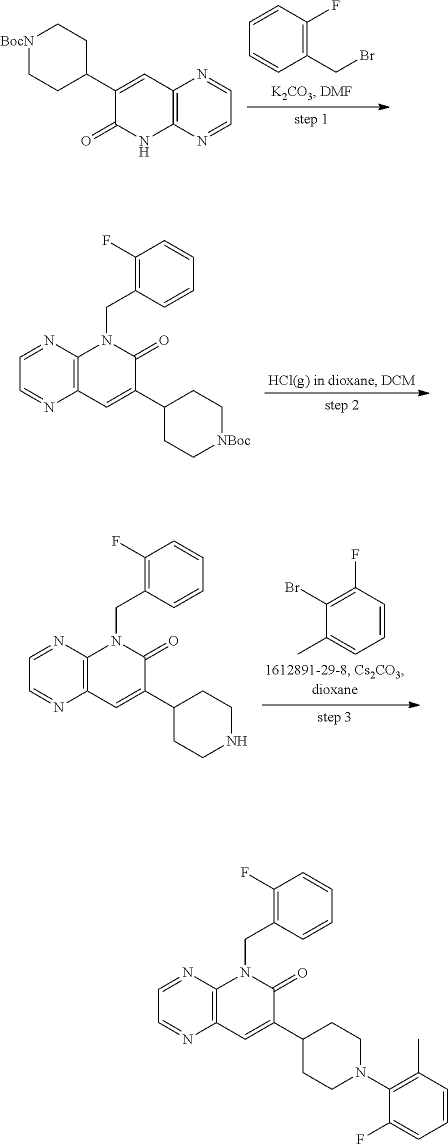

Step 1: tert-butyl 4-(5-(2-fluorobenzyl)-6-oxo-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (60 mg, 0.182 mmol, 1 equiv) and 1-(bromomethyl)-2-fluorobenzene (51.4 mg, 0.273 mmol, 1.5 equiv) as the starting materials to give tert-butyl 4-(5-(2-fluorobenzyl)-6-oxo-5,6-dihydropyrido[2,3-b]pyrazin-7-yl)piperidine-1-carboxylate (70 mg, 87%) as a light yellow solid. MS m/z: 439 [M+H]⁺.

Step 2: 5-(2-fluorobenzyl)-7-(piperidin-4-yl)pyrido[2,3-b]pyrazin-6(5H)-one: Followed general procedure F using tert-butyl 4-(5-(2-fluorobenzyl)-6-oxo-5,6-dihydropyrido[2,3-b]pyrazin-7-yl)piperidine-1-carboxylate (70 mg, 0.16 mmol, 1 equiv) as the starting material to give the crude product 5-(2-fluorobenzyl)-7-(piperidin-4-yl)pyrido[2,3-b]pyrazin-6(5H)-one (50 mg) as an off-white solid. MS m/z: 339 [M+H]⁺.

Step 3: 7-(1-(2-fluoro-6-methylphenyl)piperidin-4-yl)-5-(2-fluorobenzyl)pyrido[2,3-b]pyrazin-6(5H)-one: Followed general procedure G using 5-(2-fluorobenzyl)-7-(piperidin-4-yl)pyrido[2,3-b]pyrazin-6(5H)-one hydrochloride (50 mg, 0.148 mmol, 1 equiv) and 2-bromo-1-fluoro-3-methylbenzene (41.9 mg, 0.222 mmol, 1.5 equiv) as the starting materials to give 7-(1-(2-fluoro-6-methylphenyl)piperidin-4-yl)-5-(2-fluorobenzyl)pyrido[2,3-b]pyrazin-6(5H)-one (30.3 mg, 42.4%) as a light yellow solid. ¹H NMR (400 MHz, Chloroform-d) δ 8.53-8.41 (m, 2H), 7.86 (s, 1H), 7.25-7.18 (m, 1H), 7.10-7.05 (m, 1H), 7.04-6.95 (m, 4H), 6.92-6.84 (m, 1H), 5.80 (s, 2H), 3.38 (t, 2H), 3.27-3.09 (m, 3H), 2.39 (s, 3H), 2.10-1.99 (m, 2H), 1.95-1.81 (m, 2H). MS m/z: 447.25 [M+H]⁺.

### 3-(1-(2-Fluoro-6-methylphenyl)piperidin-4-yl)-1-((3-(trifluoromethyl)pyridin-2-yl)methyl)quinazoline-2,4(1H,3H)-dione (36)

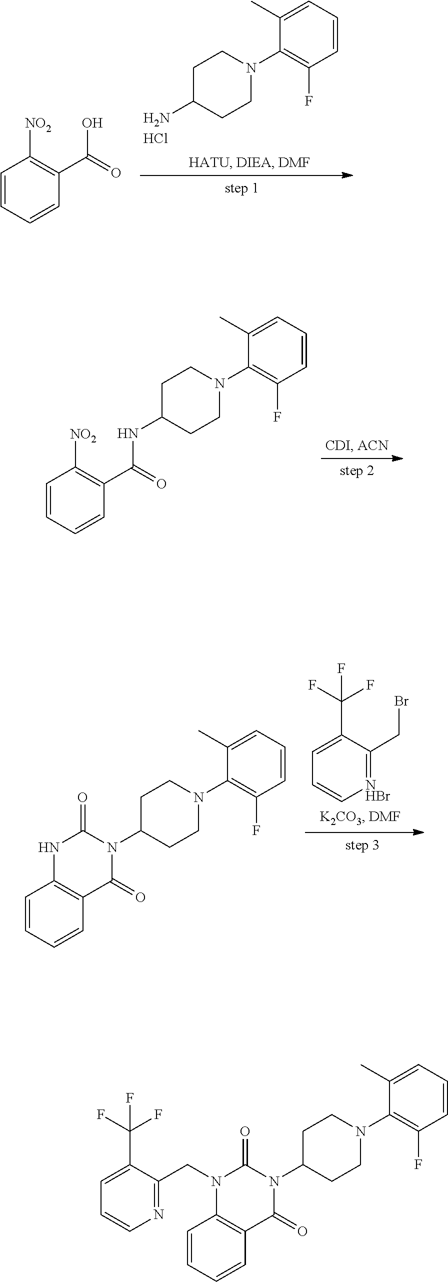

Step 1: N-(1-(2-fluoro-6-methylphenyl)piperidin-4-yl)-2-nitrobenzamide: Followed general procedure A using tert-butyl 4-(8-methyl-6-oxo-5,6-dihydropyrido[2,3-b]pyrazin-7-yl)piperidine-1-carboxylate (100 mg, 0.598 mmol, 1 equiv) and 1-(2-fluoro-6-methylphenyl)piperidin-4-amine hydrochloride (146 mg, 0.598 mmol, 1 equiv) as the starting materials to give N-(1-(2-fluoro-6-methylphenyl)piperidin-4-yl)-2-nitrobenzamide (100 mg, 46%) as a yellow oil. MS m/z: 358 [M+H]⁺.

Step 2: 2-amino-N-(1-(2-fluoro-6-methylphenyl)piperidin-4-yl)benzamide: Followed general procedure K using N-(1-(2-fluoro-6-methylphenyl)piperidin-4-yl)-2-nitrobenzamide (100 mg, 0.28 mmol, 1 equiv) as the starting material to give the crude product 2-amino-N-(1-(2-fluoro-6-methylphenyl)piperidin-4-yl)benzamide (70 mg) as a yellow oil. MS m/z: 328 [M+H]⁺.

Step 3: 3-(1-(2-fluoro-6-methylphenyl)piperidin-4-yl)quinazoline-2,4(1H,3H)-dione: Followed general procedure L using 2-amino-N-(1-(2-fluoro-6-methylphenyl)piperidin-4-yl)benzamide (70 mg, 0.214 mmol, 1 equiv) as the starting material to give 3-(1-(2-fluoro-6-methylphenyl)piperidin-4-yl)quinazoline-2,4(1H,3H)-dione (60 mg, 79%) as a yellow oil. MS m/z: 354 [M+H]⁺.

Step 4: 3-(1-(2-fluoro-6-methylphenyl)piperidin-4-yl)-1-((3-(trifluoromethyl)pyridin-2-yl)methyl)quinazoline-2,4(1H,3H)-dione: Followed general procedure E using 3-(1-(2-fluoro-6-methylphenyl)piperidin-4-yl)quinazoline-2,4(1H,3H)-dione (60 mg, 0.17 mmol, 1 equiv) and 2-(bromomethyl)-3-(trifluoromethyl)pyridine hydrobromide (59.9 mg, 0.187 mmol, 1.1 equiv) as the starting materials to give 3-(1-(2-fluoro-6-methylphenyl)piperidin-4-yl)-1-((3-(trifluoromethyl)pyridin-2-yl)methyl)quinazoline-2,4(1H,3H)-dione (21.9 mg, 25.1%) as a white solid. ¹H NMR (400 MHz, Chloroform-d) δ 8.56 (d, 1H), 8.27 (dd, 1H), 8.04-7.97 (m, 1H), 7.54-7.45 (m, 1H), 7.34-7.30 (m, 1H), 7.23-7.19 (m, 1H), 7.05-6.95 (m, 2H), 6.92-6.80 (m, 2H), 5.67 (s, 2H), 5.23-5.13 (m, 1H), 3.35 (t, 2H), 3.11 (d, 2H), 3.06-2.92 (m, 2H), 2.40 (s, 3H), 1.79 (d, 2H). MS m/z: 513.25 [M+H]⁺.

### 3-(1-(2-Fluoro-6-methylphenyl)piperidin-4-yl)-1-((3-(trifluoromethyl)pyrazin-2-yl)methyl)pteridine-2,4(1H,3H)-dione (37)

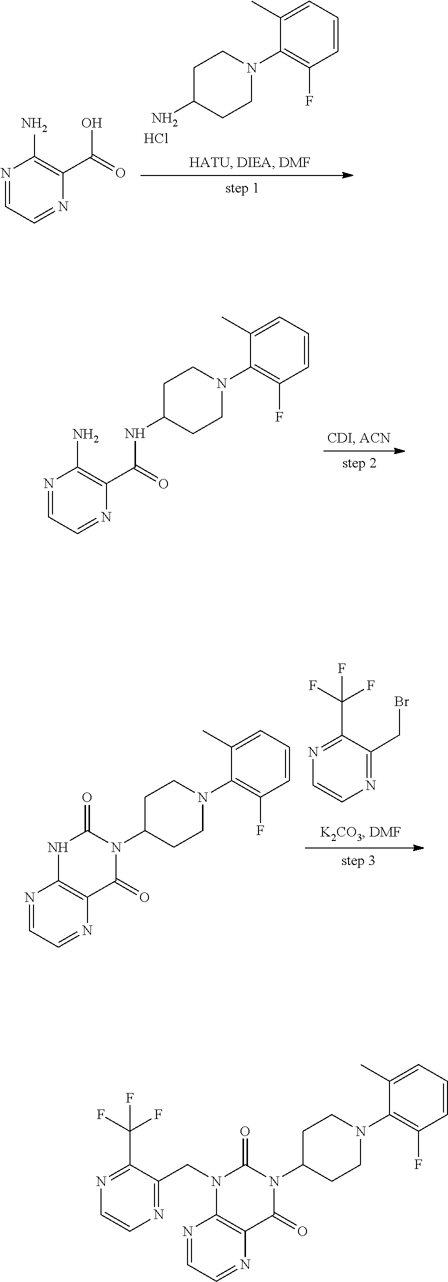

Step 1: 3-amino-N-(1-(2-fluoro-6-methylphenyl)piperidin-4-yl)pyrazine-2-carboxamide: Followed general procedure A using 3-aminopyrazine-2-carboxylic acid (136 mg, 0.908 mmol, 1.2 equiv) and 1-(2-fluoro-6-methylphenyl)piperidin-4-amine hydrochloride (200 mg, 0.817 mmol, 1 equiv) as the starting materials to give 3-amino-N-(1-(2-fluoro-6-methylphenyl)piperidin-4-yl)pyrazine-2-carboxamide (290 mg, 64%) as a yellow solid. MS m/z: 330 [M+H]⁺.

Step 2: 3-(1-(2-fluoro-6-methylphenyl)piperidin-4-yl)pteridine-2,4(1H,3H)-dione

Followed general procedure L using 3-amino-N-(1-(2-fluoro-6-methylphenyl)piperidin-4-yl)pyrazine-2-carboxamide (100 mg, 0.304 mmol, 1 equiv) as the starting material to give 3-(1-(2-fluoro-6-methylphenyl)piperidin-4-yl)pteridine-2,4(1H,3H)-dione (40 mg, 37%) as a white solid. MS m/z: 356 [M+H]⁺.

Step 3: 3-(1-(2-fluoro-6-methylphenyl)piperidin-4-yl)-1-((3-(trifluoromethyl)pyrazin-2-yl)methyl)pteridine-2,4(1H,3H)-dione: Followed general procedure G using 3-(1-(2-fluoro-6-methylphenyl)piperidin-4-yl)pteridine-2,4(1H,3H)-dione (40 mg, 0.113 mmol, 1 equiv) and 2-(bromomethyl)-3-(trifluoromethyl)pyrazine (29.8 mg, 0.124 mmol, 1.1 equiv) as the starting materials to give 3-(1-(2-fluoro-6-methylphenyl)piperidin-4-yl)-1-((3-(trifluoromethyl)pyrazin-2-yl)methyl)pteridine-2,4(1H,3H)-dione (21.2 mg, 34.9%) as a light yellow solid. ¹H NMR (400 MHz, Chloroform-d) δ 8.60 (d, 1H), 8.57 (d, 1H), 8.50 (d, 1H), 8.46 (d, 1H), 7.03-6.92 (m, 2H), 6.91-6.81 (m, 1H), 5.91 (s, 2H), 5.21-5.01 (m, 1H), 3.35 (t, 2H), 3.11 (d, 2H), 3.00-2.86 (m, 2H), 2.38 (s, 3H), 1.77 (d, 2H). MS m/z: 516.20 [M+H]⁺.

### 7-(1-(2-(Trifluoromethyl)phenyl)piperidin-4-yl)-5-((3-(trifluoromethyl)pyridin-2-yl)methyl)pyrido[2,3-b]pyrazin-6(5H)-one (38)

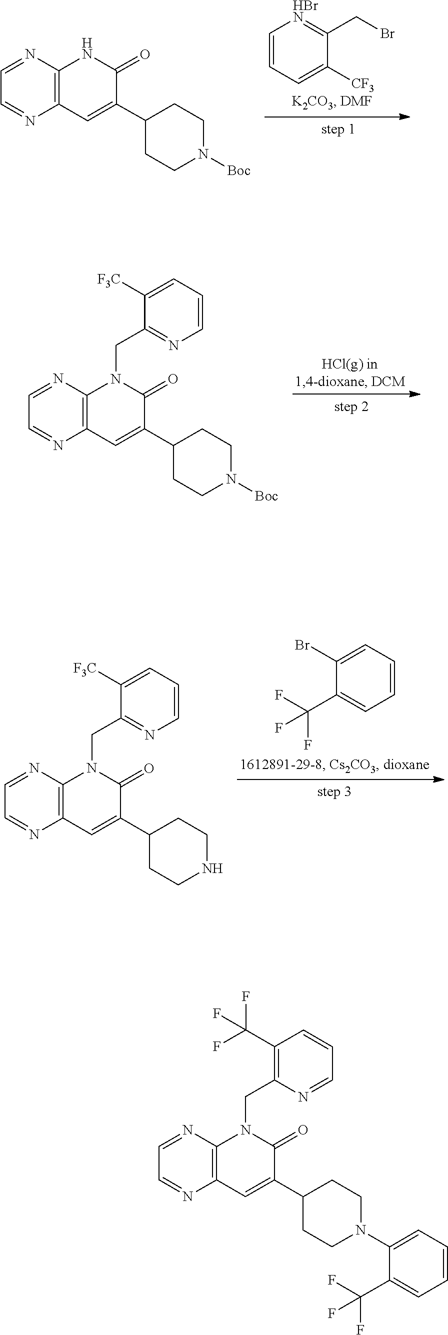

Step 1: tert-butyl 4-(6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (50 mg, 0.151 mmol, 1 equiv) and 2-(bromomethyl)-3-(trifluoromethyl)pyridine hydrogen bromide (48.5 mg, 0.151 mmol, 1 equiv) as the starting materials to give tert-butyl 4-(6-oxo-5-((3-(trifluoromethyl)pyridin-2-yl)methyl)-5,6-dihydropyrido[2,3-b]pyrazin-7-yl)piperidine-1-carboxylate (50 mg, 69%) as a yellow solid. MS m/z: 490 [M+H]⁺.

Step 2: 7-(piperidin-4-yl)-5-((3-(trifluoromethyl)pyridin-2-yl)methyl)pyrido[2,3-b]pyrazin-6(5H)-one: Followed general procedure F using tert-butyl 4-(6-oxo-5-((3-(trifluoromethyl)pyridin-2-yl)methyl)-5,6-dihydropyrido[2,3-b]pyrazin-7-yl)piperidine-1-carboxylate (50 mg, 0.102 mmol, 1 equiv) as the starting material to give the crude product 7-(piperidin-4-yl)-5-((3-(trifluoromethyl)pyridin-2-yl)methyl)pyrido[2,3-b]pyrazin-6(5H)-one (34.6 mg, 86%) as a white solid. MS m/z: 390 [M+H]⁺.

Step 3: 7-(1-(2-(trifluoromethyl)phenyl)piperidin-4-yl)-5-((3-(trifluoromethyl)pyridin-2-yl)methyl)pyrido[2,3-b]pyrazin-6(5H)-one: Followed general procedure G using 7-(piperidin-4-yl)-5-((3-(trifluoromethyl)pyridin-2-yl)methyl)pyrido[2,3-b]pyrazin-6(5H)-one (35 mg, 0.09 mmol, 1 equiv) and 1-bromo-2-(trifluoromethyl)benzene (30.3 mg, 0.135 mmol, 1.5 equiv) as the starting materials to give 7-(1-(2-(trifluoromethyl)phenyl)piperidin-4-yl)-5-((3-(trifluoromethyl)pyridin-2-yl)methyl)pyrido[2,3-b]pyrazin-6(5H)-one (18.2 mg, 37.8%) as an off-white solid. ¹H NMR (400 MHz, Chloroform-d) δ 8.48 (dd, 1H), 8.42 (d, 1H), 8.37 (dd, 1H), 7.98 (d, 1H), 7.92 (d, 1H), 7.64 (d, 1H), 7.52 (t, 1H), 7.42 (d, 1H), 7.26-7.20 (m, 2H), 6.05 (s, 2H), 3.25-3.10 (m, 3H), 2.94 (t, 2H), 2.07 (d, 2H), 1.97-1.84 (m, 2H). MS m/z: 534.25 [M+H]⁺.

### 3-(1-(2-Fluoro-6-methylphenyl)piperidin-4-yl)-1-(2-(trifluoromethyl)benzyl)pyrazino[2,3-b]pyrazin-2(1H)-one (39)

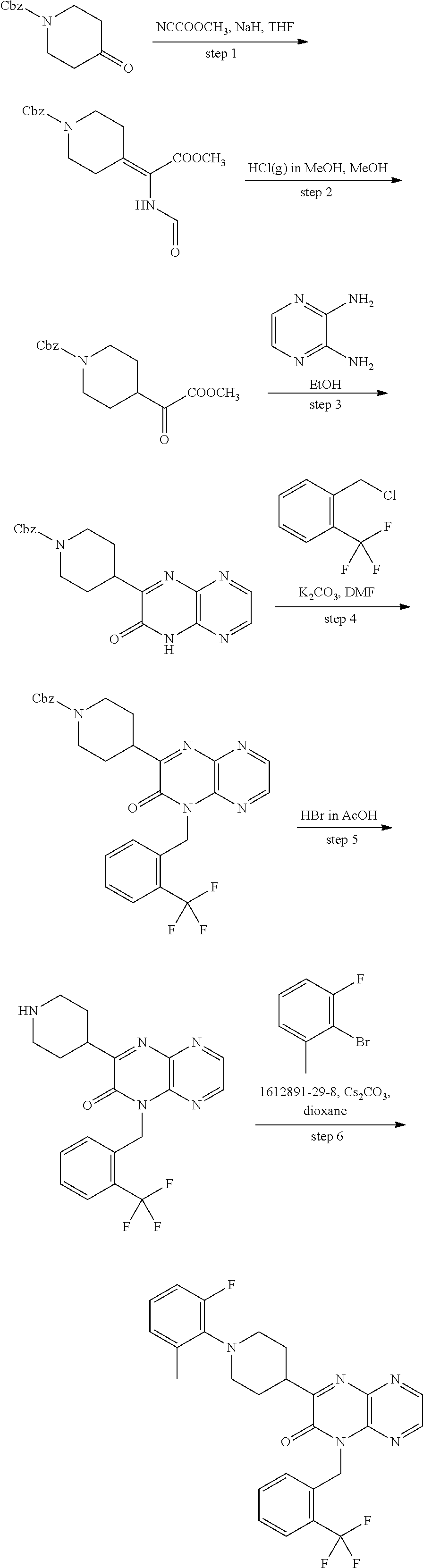

Step 1: benzyl 4-(1-formamido-2-methoxy-2-oxoethylidene)piperidine-1-carboxylate: Followed general procedure N using 2-methoxy-2-oxoacetonitrile (4.74 g, 55.7 mmol, 1.3 equiv) and benzyl 4-oxopiperidine-1-carboxylate (10 g, 42.9 mmol, 1 equiv) as the starting materials to give benzyl 4-(1-formamido-2-methoxy-2-oxoethylidene)piperidine-1-carboxylate (3 g, 31%) as an off-white solid. MS m/z: 333 [M+H]⁺.

Step 2: benzyl 4-(2-methoxy-2-oxoacetyl)piperidine-1-carboxylate: Followed general procedure O using benzyl 4-(1-formamido-2-methoxy-2-oxoethylidene)piperidine-1-carboxylate (1 g, 3.01 mmol, 1 equiv) as the starting material to give the crude product benzyl 4-(2-methoxy-2-oxoacetyl)piperidine-1-carboxylate (1 g) as a yellow oil. MS m/z: 306 [M+H]⁺.

Step 3: benzyl 4-(3-oxo-3,4-dihydropyrazino[2,3-b]pyrazin-2-yl)piperidine-1-carboxylate: Followed general procedure P using benzyl 4-(2-methoxy-2-oxoacetyl)piperidine-1-carboxylate (1 g, 3.27 mmol, 1 equiv) and pyrazine-2,3-diamine (0.36 g, 3.27 mmol, 1 equiv) as the starting materials to give benzyl 4-(3-oxo-3,4-dihydropyrazino[2,3-b]pyrazin-2-yl)piperidine-1-carboxylate (250 mg, 20%) as a yellow solid. MS m/z: 366 [M+H]⁺.

Step 4: benzyl 4-(3-oxo-3,4-dihydropyrazino[2,3-b]pyrazin-2-yl)piperidine-1-carboxylate: Followed general procedure E using benzyl 4-(3-oxo-3,4-dihydropyrazino[2,3-b]pyrazin-2-yl)piperidine-1-carboxylate (250 mg, 0.684 mmol, 1 equiv) and 1-(chloromethyl)-2-(trifluoromethyl)benzene (199 mg, 1.02 mmol, 1.5 equiv) as the starting materials to give benzyl 4-(3-oxo-4-(2-(trifluoromethyl)benzyl)-3,4-dihydropyrazino[2,3-b]pyrazin-2-yl)piperidine-1-carboxylate (150 mg, 41%) as a yellow solid. MS m/z: 524 [M+H]⁺.

Step 5: 3-(piperidin-4-yl)-1-(2-(trifluoromethyl)benzyl)pyrazino[2,3-b]pyrazin-2(1H)-one: Followed general procedure Q using benzyl 4-(3-oxo-4-(2-(trifluoromethyl)benzyl)-3,4-dihydropyrazino[2,3-b]pyrazin-2-yl)piperidine-1-carboxylate (100 mg, 0.191 mmol, 1 equiv) as the starting material to give the crude product 3-(piperidin-4-yl)-1-(2-(trifluoromethyl)benzyl)pyrazino[2,3-b]pyrazin-2(1H)-one (70 mg) as a yellow oil. MS m/z: 390 [M+H]⁺.

Step 6: 3-(1-(2-fluoro-6-methylphenyl)piperidin-4-yl)-1-(2-(trifluoromethyl)benzyl)pyrazino[2,3-b]pyrazin-2(1H)-one: Followed general procedure G using 3-(piperidin-4-yl)-1-(2-(trifluoromethyl)benzyl)pyrazino[2,3-b]pyrazin-2(1H)-one (50 mg, 0.128 mmol, 1 equiv) and 2-bromo-1-fluoro-3-methylbenzene (36.4 mg, 0.192 mmol, 1.5 equiv) as the starting materials to give 3-(1-(2-fluoro-6-methylphenyl)piperidin-4-yl)-1-(2-(trifluoromethyl)benzyl)pyrazino[2,3-b]pyrazin-2(1H)-one (23.6 mg, 36.7%) as a yellow solid. ¹H NMR (400 MHz, Chloroform-d) δ 8.61 (d, 1H), 8.46 (d, 1H), 7.77-7.70 (m, 1H), 7.39-7.32 (m, 2H), 7.03-6.95 (m, 2H), 6.92-6.84 (m, 1H), 6.76-6.65 (m, 1H), 5.89 (s, 2H), 3.65-3.53 (m, 1H), 3.45-3.27 (m, 2H), 3.22-3.13 (m, 2H), 2.37 (s, 3H), 2.19-2.07 (m, 4H). MS m/z: 498.2 [M+H]⁺.

### (R)-7-(1-(2-Fluoro-6-methylphenyl)piperidin-4-yl)-5-(5,6,7,8-tetrahydroquinolin-8-yl)pyrido[2,3-b]pyrazin-6(5H)-one (40a); (S)-7-(1-(2-Fluoro-6-methylphenyl)piperidin-4-yl)-5-(5,6,7,8-tetrahydroquinolin-8-yl)pyrido[2,3-b]pyrazin-6(5H)-one (40b)

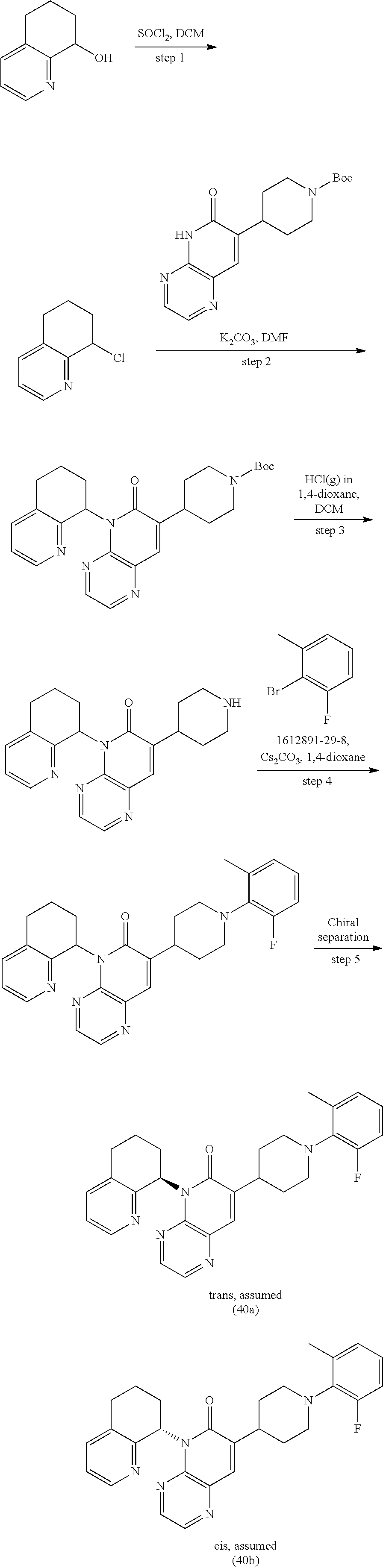

Step 1: 8-chloro-5,6,7,8-tetrahydroquinoline: Followed general procedure J using 5,6,7,8-tetrahydroquinolin-8-ol (500 mg, 3.35 mmol, 1 equiv) as the starting material to give the crude product 8-chloro-5,6,7,8-tetrahydroquinoline (350 mg) as a yellow oil. MS m/z: 168 [M+H]⁺

Step 2: tert-butyl 4-(6-oxo-5-(5,6,7,8-tetrahydroquinolin-8-yl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (300 mg, 0.908 mmol, 1 equiv) and 8-chloro-5,6,7,8-tetrahydroquinoline (197 mg, 1.18 mmol, 1.3 equiv) as the starting materials to give tert-butyl 4-(6-oxo-5-(5,6,7,8-tetrahydroquinolin-8-yl)-5,6-dihydropyrido[2,3-b]pyrazin-7-yl)piperidine-1-carboxylate (200 mg, 47%) as a yellow oil. MS m/z: 462 [M+H]⁺.

Step 3: 7-(piperidin-4-yl)-5-(5,6,7,8-tetrahydroquinolin-8-yl)pyrido[2,3-b]pyrazin-6(5H)-one: Followed general procedure F using tert-butyl 4-(6-oxo-5-(5,6,7,8-tetrahydroquinolin-8-yl)-5,6-dihydropyrido[2,3-b]pyrazin-7-yl)piperidine-1-carboxylate (200 mg, 0.433 mmol, 1 equiv) as the starting material to give the crude product 7-(piperidin-4-yl)-5-(5,6,7,8-tetrahydroquinolin-8-yl)pyrido[2,3-b]pyrazin-6(5H)-one (140 mg) as a yellow oil. MS m/z: 362 [M+H]⁺.

Step 4: 7-(1-(2-fluoro-6-methylphenyl)piperidin-4-yl)-5-(5,6,7,8-tetrahydroquinolin-8-yl)pyrido[2,3-b]pyrazin-6(5H)-one: Followed general procedure G using 7-(piperidin-4-yl)-5-(5,6,7,8-tetrahydroquinolin-8-yl)pyrido[2,3-b]pyrazin-6(5H)-one (140 mg, 0.387 mmol, 1 equiv) and 2-bromo-1-fluoro-3-methylbenzene (87 mg, 0.464 mmol, 1.2 equiv) as the starting materials to give the crude product 7-(1-(2-fluoro-6-methylphenyl)piperidin-4-yl)-5-(5,6,7,8-tetrahydroquinolin-8-yl)pyrido[2,3-b]pyrazin-6(5H)-one (100 mg) as a light yellow solid. The crude product was purified by prep-HPLC with the following conditions (Column: CHIRAL ART Amylose-SA, 2*25 cm, 5 m; Mobile Phase A: Hex (10 mM NH₃-MeOH), Mobile Phase B: EtOH-HPLC; Flow rate: 20 mL/min; Gradient: 40% B to 40% B in 10 min; Wave Length: 329/217 nm; RT1 (min): 4.8; RT2 (min): 8.0; Sample Solvent: MeOH:DCM=1:2; Injection Volume: 0.6 mL; Number Of Runs: 4) to afford (R)-7-(1-(2-fluoro-6-methylphenyl)piperidin-4-yl)-5-(5,6,7,8-tetrahydroquinolin-8-yl)pyrido[2,3-b]pyrazin-6(5H)-one (40a, 34.1 mg, 18.0%) as a white solid and (S)-7-(1-(2-fluoro-6-methylphenyl)piperidin-4-yl)-5-(5,6,7,8-tetrahydroquinolin-8-yl)pyrido[2,3-b]pyrazin-6(5H)-one (40b, 30.3 mg, 16.3%) as a white solid.

(R)-7-(1-(2-fluoro-6-methylphenyl)piperidin-4-yl)-5-(5,6,7,8-tetrahydroquinolin-8-yl)pyrido[2,3-b]pyrazin-6(5H)-one (40a)¹H NMR (400 MHz, Chloroform-d) δ 8.84 (d, 1H), 8.72 (d, 1H), 8.50 (d, 1H), 8.14 (s, 1H), 7.56 (s, 1H), 7.23 (s, 1H), 6.96-6.92 (m, 2H), 6.87-6.80 (m, 2H), 3.25-3.09 (m, 2H), 3.06-2.96 (m, 4H), 2.91-2.83 (m, 1H), 2.52-2.45 (m, 1H), 2.37-2.32 (m, 1H), 2.29 (s, 3H), 2.10-1.94 (m, 4H), 1.91-1.80 (m, 2H). MS m/z: 470.15 [M+H]+

(S)-7-(1-(2-fluoro-6-methylphenyl)piperidin-4-yl)-5-(5,6,7,8-tetrahydroquinolin-8-yl)pyrido[2,3-b]pyrazin-6(5H)-one (40b)¹H NMR (400 MHz, Chloroform-d) δ 8.84 (d, 1H), 8.73 (d, 1H), 8.53 (s, 1H), 8.16 (s, 1H), 7.64 (s, 1H), 7.29 (s, 1H), 7.00-6.92 (m, 2H), 6.87-6.79 (m, 2H), 3.25-2.98 (m, 6H), 2.93-2.85 (m, 1H), 2.51-2.44 (m, 1H), 2.37 (s, 1H), 2.29 (s, 3H), 2.13-2.04 (m, 2H), 2.00-1.96 (m, 2H), 1.90-1.78 (m, 2H). MS m/z: 470.2 [M+H]⁺

### 7-(1-(2-Fluoro-6-methylphenyl)piperidin-4-yl)-3-methyl-5-((3-(trifluoromethyl)pyridin-2-yl)methyl)pyrido[2,3-b]pyrazin-6(5H)-one (41)

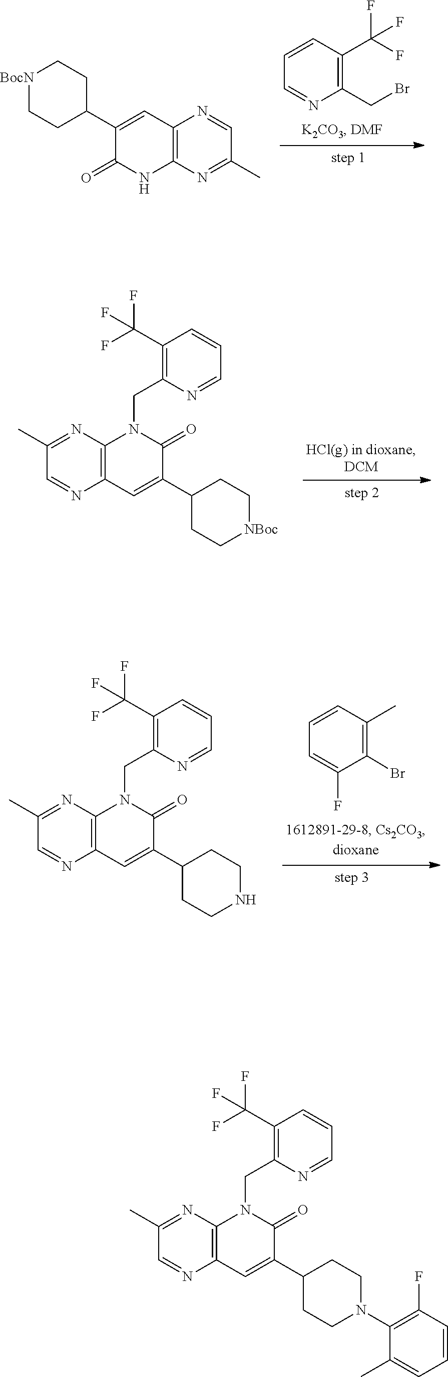

Step 1: tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (90 mg, 0.261 mmol, 1 equiv) and 2-(bromomethyl)-3-(trifluoromethyl)pyridine (62.7 mg, 0.261 mmol, 1.5 equiv) as the starting materials to give tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (55 mg, 48%) as an off-white solid. MS m/z: 504 [M+H]⁺.

Step 2: 3-methyl-7-(piperidin-4-yl)-5-((3-(trifluoromethyl)pyridin-2-yl)methyl)pyrido[2,3-b]pyrazin-6(5H)-one: Followed general procedure F using tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (55 mg, 0.109 mmol, 1 equiv) as the starting material to give the crude product 3-methyl-7-(piperidin-4-yl)-5-((3-(trifluoromethyl)pyridin-2-yl)methyl)pyrido[2,3-b]pyrazin-6(5H)-one (35 mg) as an off-white solid. MS m/z: 404 [M+H]⁺.

Step 3: 7-(1-(2-fluoro-6-methylphenyl)piperidin-4-yl)-3-methyl-5-((3-(trifluoromethyl)pyridin-2-yl)methyl)pyrido[2,3-b]pyrazin-6(5H)-one: Followed general procedure G using 3-methyl-7-(piperidin-4-yl)-5-((3-(trifluoromethyl)pyridin-2-yl)methyl)pyrido[2,3-b]pyrazin-6(5H)-one (35 mg, 0.087 mmol, 1 equiv) and 3-bromo-5-fluoro-2-methylpyridine (19.6 mg, 0.104 mmol, 1.2 equiv) as the starting materials to give 7-(1-(2-fluoro-6-methylphenyl)piperidin-4-yl)-3-methyl-5-((3-(trifluoromethyl)pyridin-2-yl)methyl)pyrido[2,3-b]pyrazin-6(5H)-one (4.4 mg, 9.1%) as an off-white solid. ¹H NMR (300 MHz, Chloroform-d) δ 8.46-8.32 (m, 2H), 8.01-7.88 (m, 2H), 7.24-7.20 (m, 1H), 7.10-6.87 (m, 3H), 6.04 (s, 2H), 3.54-3.40 (m, 2H), 3.33-3.15 (m, 3H), 2.52 (s, 6H), 2.20-2.04 (m, 4H). MS m/z: 512.15 [M+H]⁺.

### 3-(1-(2-Fluoro-6-methylphenyl)piperidin-4-yl)-1-((3-(trifluoromethyl)pyridin-2-yl)methyl)pteridine-2,4(1H,3H)-dione (42)

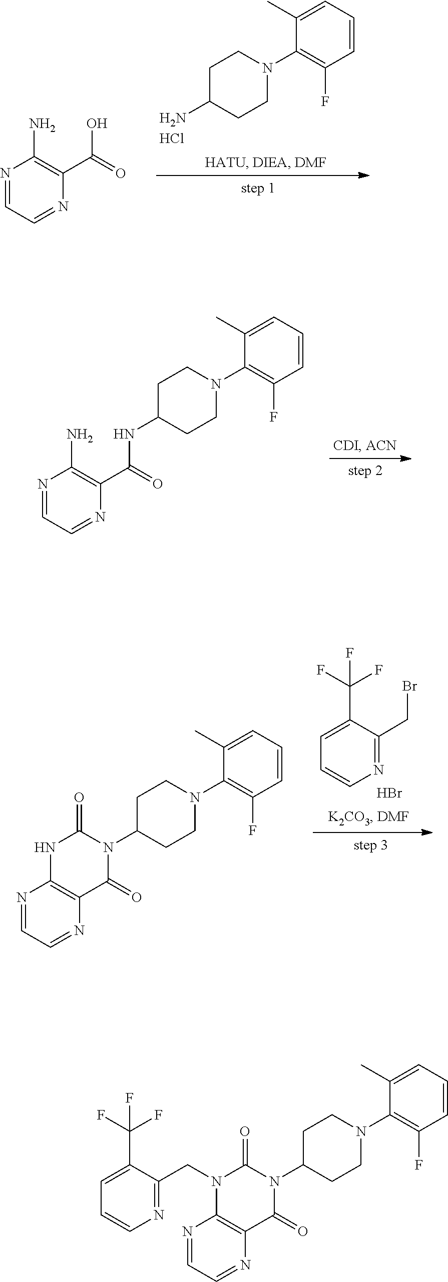

Step 1: 3-amino-N-(1-(2-fluoro-6-methylphenyl)piperidin-4-yl)pyrazine-2-carboxamide: Followed general procedure A using 3-aminopyrazine-2-carboxylic acid (100 mg, 0.719 mmol, 1 equiv) and 1-(2-fluoro-6-methylphenyl)piperidin-4-amine hydrochloride (176 mg, 0.719 mmol, 1 equiv) as the starting materials to give 3-amino-N-(1-(2-fluoro-6-methylphenyl)piperidin-4-yl)pyrazine-2-carboxamide (140 mg, 59%) as a yellow oil. MS m/z: 330 [M+H]⁺.

Step 2: 3-(1-(2-fluoro-6-methylphenyl)piperidin-4-yl)pteridine-2,4(1H,3H)-dione: Followed general procedure L using 2-amino-N-(1-(2-fluoro-6-methylphenyl)piperidin-4-yl)benzamide (140 mg, 0.425 mmol, 1.00 equiv) as the starting material to give the crude product 3-(1-(2-fluoro-6-methylphenyl)piperidin-4-yl)pteridine-2,4(1H,3H)-dione (60 mg, 39%) as a yellow oil. MS m/z: 356 [M+H]⁺.

Step 3: 3-(1-(2-fluoro-6-methylphenyl)piperidin-4-yl)-1-((3-(trifluoromethyl)pyridin-2-yl)methyl)pteridine-2,4(1H,3H)-dione: Followed general procedure E using 3-(1-(2-fluoro-6-methylphenyl)piperidin-4-yl)pteridine-2,4(1H,3H)-dione (60 mg, 0.169 mmol, 1 equiv) and 2-(bromomethyl)-3-(trifluoromethyl)pyridine hydrobromide (59.6 mg, 0.186 mmol, 1.1 equiv) as the starting materials to give 3-(1-(2-fluoro-6-methylphenyl)piperidin-4-yl)-1-((3-(trifluoromethyl)pyridin-2-yl)methyl)pteridine-2,4(1H,3H)-dione (12.6 mg, 14.5%) as a white solid. ¹H NMR (400 MHz, Chloroform-d) δ 8.57 (d, 1H), 8.52-8.44 (m, 2H), 8.04-7.97 (m, 1H), 7.31 (dd, 2H), 7.04-6.92 (m, 2H), 6.92-6.82 (m, 1H), 5.87 (s, 2H), 5.20 (d, 1H), 3.37 (t, 2H), 3.14 (d, 2H), 3.04-2.90 (m, 2H), 2.40 (s, 3H), 1.80 (d, 2H). MS m/z: 515.20 [M+H]⁺.

### (R)-7-(1-(2-Fluoro-6-methylphenyl)piperidin-4-yl)-5-(5,6,7,8-tetrahydroquinoxalin-5-yl)pyrido[2,3-b]pyrazin-6(5H)-one (43a); (S)-7-(1-(2-Fluoro-6-methylphenyl)piperidin-4-yl)-5-(5,6,7,8-tetrahydroquinoxalin-5-yl)pyrido[2,3-b]pyrazin-6(5H)-one (43b)

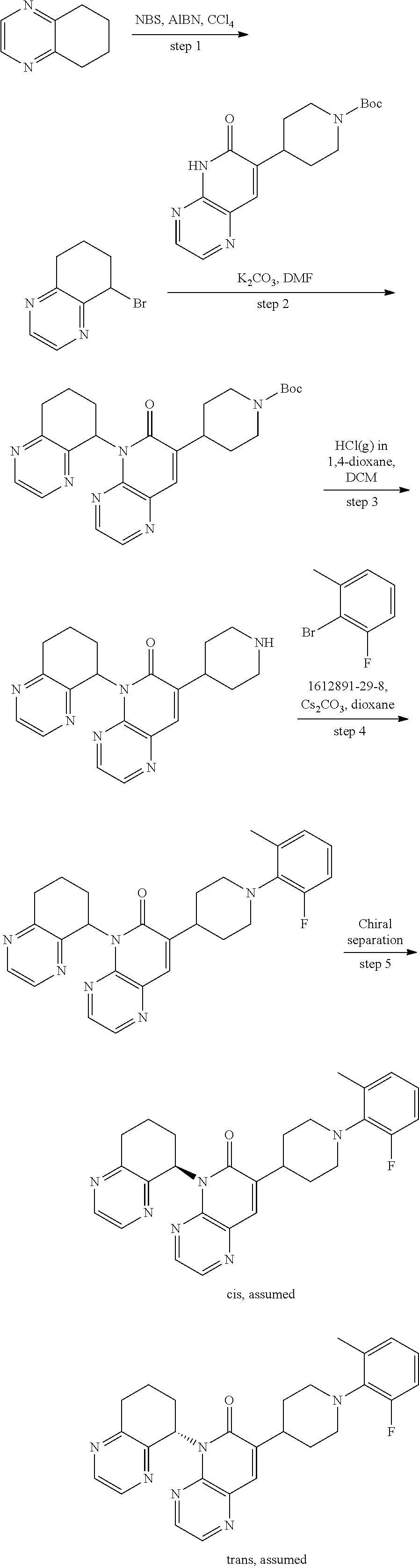

Step 1: 5-bromo-5,6,7,8-tetrahydroquinoxaline: Followed general procedure E using tert-butyl 4-(3-methyl-6-oxo-5,6-dihydropyrido[2,3-b]pyrazin-7-yl)piperidine-1-carboxylate (260 mg, 0.755 mmol, 1 equiv) and 5-bromo-5,6,7,8-tetrahydroquinoxaline (241 mg, 1.13 mmol, 1.5 equiv) as the starting materials to give tert-butyl 4-(3-methyl-6-oxo-5-(5,6,7,8-tetrahydroquinoxalin-5-yl)-5,6-dihydropyrido[2,3-b]pyrazine-7-yl) piperidine-1-carboxylate (160 mg, 44%) as a yellow oil. MS m/z: 213 [M+H]⁺.

Step 2: tert-butyl 4-(6-oxo-5-(5,6,7,8-tetrahydroquinoxalin-5-yl)-5,6-dihydropyrido[2,3-b]pyrazin-7-yl)piperidine-1-carboxylate: Followed general procedure E using 5-bromo-5,6,7,8-tetrahydroquinoxaline (300 mg, 0.908 mmol, 1 equiv) and tert-butyl 4-(6-oxo-5,6-dihydropyrido[2,3-b]pyrazin-7-yl)piperidine-1-carboxylate (232 mg, 1.09 mmol, 1.2 equiv) as the starting materials to give tert-butyl 4-(6-oxo-5-(5,6,7,8-tetrahydroquinoxalin-5-yl)-5,6-dihydropyrido[2,3-b]pyrazin-7-yl)piperidine-1-carboxylate (320 mg, 76%) as a yellow oil. MS m/z: 463 [M+H]⁺.

Step 3: 7-(piperidin-4-yl)-5-(5,6,7,8-tetrahydroquinoxalin-5-yl)pyrido[2,3-b]pyrazin-6(5H)-one: Followed general procedure F using tert-butyl 4-(6-oxo-5-(5,6,7,8-tetrahydroquinoxalin-5-yl)-5,6-dihydropyrido[2,3-b]pyrazin-7-yl)piperidine-1-carboxylate (320 mg, 0.692 mmol, 1 equiv) as the starting material to give the crude product 7-(piperidin-4-yl)-5-(5,6,7,8-tetrahydroquinoxalin-5-yl)pyrido[2,3-b]pyrazin-6(5H)-one (210 mg, 83%) as a yellow oil. MS m/z: 363 [M+H]⁺.

Step 4: 7-(1-(2-fluoro-6-methylphenyl)piperidin-4-yl)-5-(5,6,7,8-tetrahydroquinoxalin-5-yl)pyrido[2,3-b]pyrazin-6(5H)-one: Followed general procedure G using 7-(piperidin-4-yl)-5-(5,6,7,8-tetrahydroquinoxalin-5-yl)pyrido[2,3-b]pyrazin-6(5H)-one (200 mg, 0.552 mmol, 1 equiv) and 2-bromo-1-fluoro-3-methylbenzene (156 mg, 0.828 mmol, 1.5 equiv) as the starting materials to give the crude product 7-(1-(2-fluoro-6-methylphenyl)piperidin-4-yl)-5-(5,6,7,8-tetrahydroquinoxalin-5-yl)pyrido[2,3-b]pyrazin-6(5H)-one (80 mg) as an off-white solid. The crude product was purified by Prep-HPLC with the following conditions Column: (CHIRALPAK IA, 2*25 cm, 5 m; Mobile Phase A: Hex (10 mM NH₃-MeOH), Mobile Phase B: EtOH-HPLC; Flow rate: 20 mL/min; Gradient: 30% B to 30% B in 10 min; Wave Length: 220/203 nm; RT1 (min): 5.4725; RT2 (min): 7.21; Sample Solvent: MeOH:DCM=1:1-HPLC; Injection Volume: 0.45 mL; Number Of Runs: 5) to afford (R)-7-(1-(2-fluoro-6-methylphenyl)piperidin-4-yl)-5-(5,6,7,8-tetrahydroquinoxalin-5-yl)pyrido[2,3-b]pyrazin-6(5H)-one (43a, 24.3 mg, 8.9%) as an off-white solid and (S)-7-(1-(2-fluoro-6-methylphenyl)piperidin-4-yl)-5-(5,6,7,8-tetrahydroquinoxalin-5-yl)pyrido[2,3-b]pyrazin-6(5H)-one (43b, 15.8 mg, 5.9%) as a white solid.

(R)-7-(1-(2-fluoro-6-methylphenyl)piperidin-4-yl)-5-(5,6,7,8-tetrahydroquinoxalin-5-yl)pyrido[2,3-b]pyrazin-6(5H)-one (43a)¹H NMR (400 MHz, Chloroform-d) δ 8.40-8.07 (m, 3H), 7.80 (d, 1H), 7.05-6.72 (m, 4H), 3.43-2.94 (m, 7H), 2.67-2.52 (m, 3H), 2.38 (d, 3H), 2.30-2.21 (m, 2H), 2.18 (s, 1H), 2.13-2.02 (m, 2H), 1.97 (d, 1H), 1.85-1.75 (m, 2H). MS m/z: 485.20 [M+H]+

(S)-7-(1-(2-fluoro-6-methylphenyl)piperidin-4-yl)-5-(5,6,7,8-tetrahydroquinoxalin-5-yl)pyrido[2,3-b]pyrazin-6(5H)-one (43b)¹H NMR (400 MHz, Chloroform-d) δ 8.41-8.08 (m, 3H), 7.81 (d, 1H), 7.05-6.72 (m, 4H), 3.45-2.94 (m, 7H), 2.67-2.63 (m, 3H), 2.39 (d, 3H), 2.31-2.22 (m, 2H), 2.19 (s, 1H), 2.14-1.95 (m, 3H), 1.85-1.79 (s, 2H). MS m/z: 485.20 [M+H]⁺

### 7-(1-(5-Fluoro-2-methylpyridin-3-yl)piperidin-4-yl)-5-((3-(trifluoromethyl)pyridin-2-yl)methyl)pyrido[2,3-b]pyrazin-6(5H)-one (44)

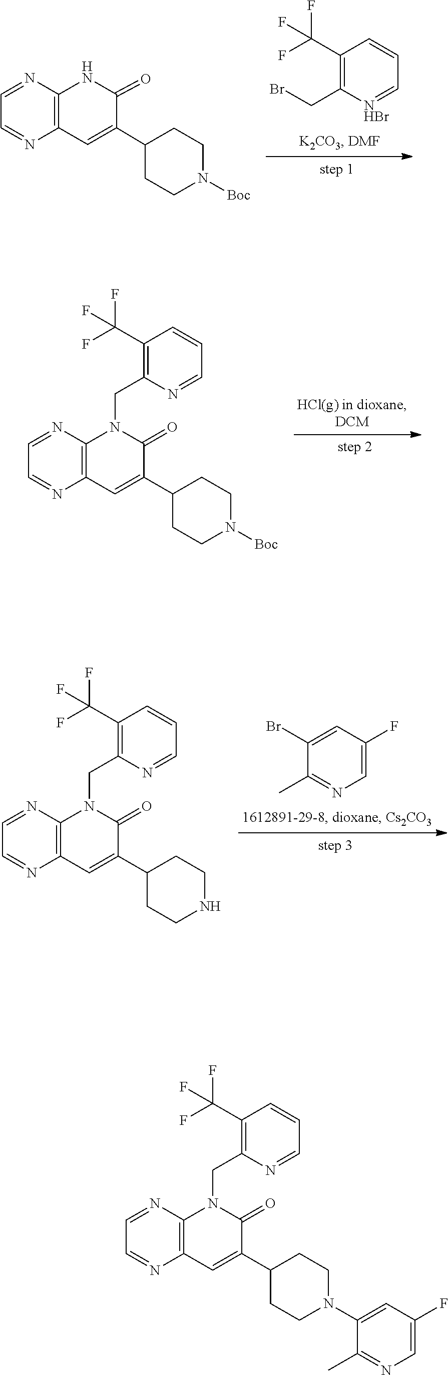

Step 1: tert-butyl 4-(6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (60 mg, 0.182 mmol, 1 equiv) and 2-(bromomethyl)-3-(trifluoromethyl)pyridine hydrobromide (58.2 mg, 0.182 mmol, 1 equiv) as the starting materials to give tert-butyl 4-(6-oxo-5-((3-(trifluoromethyl)pyridin-2-yl)methyl)-5,6-dihydropyrido[2,3-b]pyrazin-7-yl)piperidine-1-carboxylate (70 mg, 78%) as a white solid. MS m/z: 490 [M+H]⁺.

Step 2: 7-(piperidin-4-yl)-5-((3-(trifluoromethyl)pyridin-2-yl)methyl)pyrido[2,3-b]pyrazin-6(5H)-one: Followed general procedure F using tert-butyl 4-(6-oxo-5-((3-(trifluoromethyl)pyridin-2-yl)methyl)-5,6-dihydropyrido[2,3-b]pyrazin-7-yl)piperidine-1-carboxylate (70 mg, 0.143 mmol, 1 equiv) as the starting material to give the crude product 7-(piperidin-4-yl)-5-((3-(trifluoromethyl)pyridin-2-yl)methyl)pyrido[2,3-b]pyrazin-6(5H)-one (50 mg) as a white solid. MS m/z: 390 [M+H]⁺.

Step 3: 7-(1-(5-fluoro-2-methylpyridin-3-yl)piperidin-4-yl)-5-((3-(trifluoromethyl)pyridin-2-yl)methyl)pyrido[2,3-b]pyrazin-6(5H)-one: Followed general procedure G using 7-(piperidin-4-yl)-5-((3-(trifluoromethyl)pyridin-2-yl)methyl)pyrido[2,3-b]pyrazin-6(5H)-one (50 mg, 0.128 mmol, 1 equiv) and 3-bromo-5-fluoro-2-methylpyridine (26.7 mg, 0.141 mmol, 1.1 equiv) as the starting materials to give 7-(1-(5-fluoro-2-methylpyridin-3-yl)piperidin-4-yl)-5-((3-(trifluoromethyl)pyridin-2-yl)methyl)pyrido[2,3-b]pyrazin-6(5H)-one (21 mg, 31.5%) as a white solid. ¹H NMR (400 MHz, Chloroform-d) δ 8.88 (d, 1H), 8.78 (dd, 2H), 8.20 (s, 1H), 8.12-8.03 (m, 2H), 7.45 (dd, 1H), 7.12 (d, 1H), 5.99 (s, 2H), 3.29 (d, 2H), 3.16 (dd, 1H), 2.78 (t, 2H), 2.57 (s, 3H), 2.18 (d, 2H), 2.04-1.93 (m, 2H). MS m/z: 499.30 [M+H]⁺.

### 7-(1-(5-Fluoro-2-methylpyridin-3-yl)piperidin-4-yl)-5-((3-(trifluoromethyl)pyrazin-2-yl)methyl)pyrido[2,3-b]pyrazin-6(5H)-one (45)

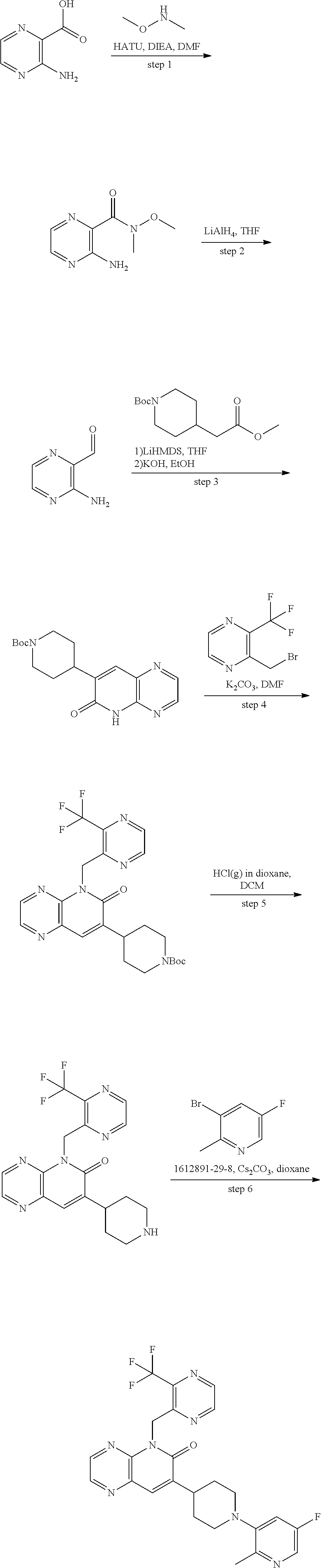

Step 1: 3-amino-N-methoxy-N-methylpyrazine-2-carboxamide: Followed general procedure A using 3-aminopyrazine-2-carboxylic acid (300 mg, 2.15 mmol, 1 equiv) and N,O-dimethylhydroxylamine (197 mg, 3.23 mmol, 1.5 equiv) as the starting materials to give 3-amino-N-methoxy-N-methylpyrazine-2-carboxamide (320 mg, 81.4%) as an off-white solid. MS m/z: 183 [M+H]⁺.

Step 2: 3-aminopyrazine-2-carbaldehyde: Followed general procedure B using 3-amino-N-methoxy-N-methylpyrazine-2-carboxamide (320 mg, 1.75 mmol, 1 equiv) and LiAlH₄ (2.11 mL, 2.107 mmol, 1.2 equiv) as the starting materials to give 3-aminopyrazine-2-carbaldehyde (200 mg, 92.4%) as a yellow solid. MS m/z: 124 [M+H]⁺.

Step 3: tert-butyl 4-(6-oxo-5,6-dihydropyrido[2,3-b]pyrazin-7-yl)piperidine-1-carboxylate: Followed general procedure D using tert-butyl 4-(2-methoxy-2-oxoethyl)piperidine-1-carboxylate (501 mg, 1.94 mmol, 1.2 equiv) and 3-amino-5-methylpyrazine-2-carbaldehyde (200 mg, 1.62 mmol, 1 equiv) as the starting materials to give tert-butyl 4-(6-oxo-5,6-dihydropyrido[2,3-b]pyrazin-7-yl)piperidine-1-carboxylate (300 mg, 55%) as a light brown solid. MS m/z: 331 [M+H]⁺.

Step 4: tert-butyl 4-(6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (90 mg, 0.272 mmol, 1 equiv) and 2-(bromomethyl)-3-(trifluoromethyl)pyrazine (98.4 mg, 0.408 mmol, 1.5 equiv) as the starting materials to give tert-butyl 4-(6-oxo-5-((3-(trifluoromethyl)pyrazin-2-yl)methyl)-5,6-dihydropyrido[2,3-b]pyrazin-7-yl)piperidine-1-carboxylate (70 mg, 52%) as an off-white solid. MS m/z: 491 [M+H]⁺.

Step 5: 7-(piperidin-4-yl)-5-((3-(trifluoromethyl)pyrazin-2-yl)methyl)pyrido[2,3-b]pyrazin-6(5H)-one: Followed general procedure F using tert-butyl 4-(6-oxo-5-((3-(trifluoromethyl)pyrazin-2-yl)methyl)-5,6-dihydropyrido[2,3-b]pyrazin-7-yl)piperidine-1-carboxylate (70 mg, 0.143 mmol, 1 equiv) as the starting material to give the crude product 7-(piperidin-4-yl)-5-((3-(trifluoromethyl)pyrazin-2-yl)methyl)pyrido[2,3-b]pyrazin-6(5H)-one (45 mg) as a yellow oil. MS m/z: 391 [M+H]⁺.

Step 6: 7-(1-(5-fluoro-2-methylpyridin-3-yl)piperidin-4-yl)-5-((3-(trifluoromethyl)pyrazin-2-yl)methyl)pyrido[2,3-b]pyrazin-6(5H)-one: Followed general procedure G using 3-methyl-7-(piperidin-4-yl)-5-((3-(trifluoromethyl)pyrazin-2-yl)methyl)pyrido[2,3-b]pyrazin-6(5H)-one (45 mg, 0.115 mmol, 1 equiv) and 3-bromo-5-fluoro-2-methylpyridine (26.2 mg, 0.138 mmol, 1.2 equiv) as the starting materials to give 7-(1-(5-fluoro-2-methylpyridin-3-yl)piperidin-4-yl)-5-((3-(trifluoromethyl)pyrazin-2-yl)methyl)pyrido[2,3-b]pyrazin-6(5H)-one (17.7 mg, 29.3%) as a light brown solid. ¹H NMR (300 MHz, Chloroform-d) δ 8.61-8.34 (m, 4H), 8.10 (d, 1H), 7.91 (s, 1H), 7.15 (d, 1H), 6.09 (s, 2H), 3.32 (d, 2H), 3.24-3.09 (m, 1H), 2.84 (t, 2H), 2.59 (s, 3H), 2.22-2.09 (m, 2H), 1.97-1.82 (m, 3H). MS m/z: 500.15 [M+H]⁺.

### 3-(1-(2-Fluoro-6-methylphenyl)piperidin-4-yl)-1-(2-(trifluoromethyl)benzyl)pyrazino[2,3-b]pyrazin-2(1H)-one (46)

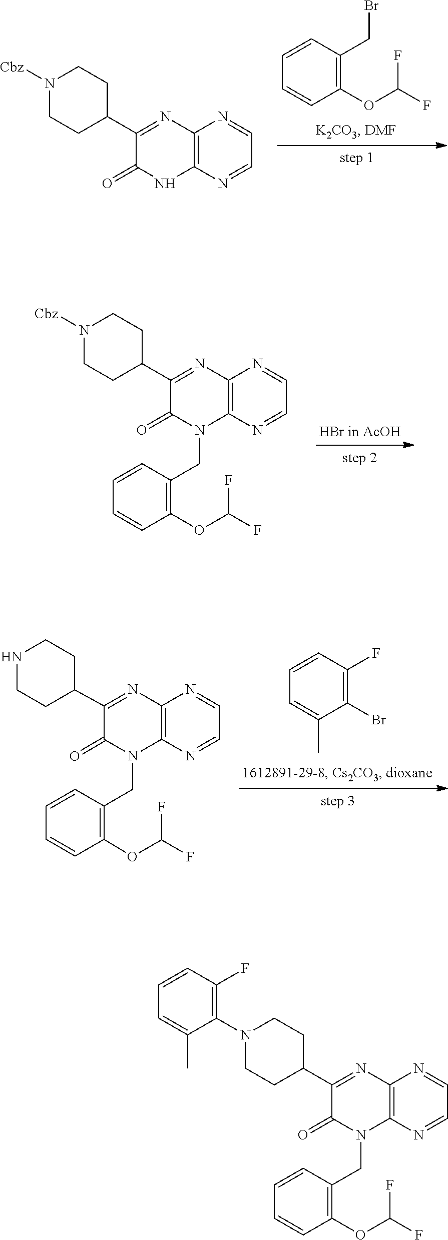

Step 1: benzyl 4-(4-(2-(difluoromethoxy)benzyl)-3-oxo-3,4-dihydropyrazino[2,3-b]pyrazin-2-yl)piperidine-1-carboxylate: Followed general procedure E using benzyl 4-(3-oxo-3,4-dihydropyrazino[2,3-b]pyrazin-2-yl)piperidine-1-carboxylate (120 mg, 0.328 mmol, 1 equiv) and 1-(bromomethyl)-2-(difluoromethoxy)benzene (116 mg, 0.492 mmol, 1.5 equiv) as the starting materials to give benzyl 4-(4-(2-(difluoromethoxy)benzyl)-3-oxo-3,4-dihydropyrazino[2,3-b]pyrazin-2-yl)piperidine-1-carboxylate (100 mg, 58%) as a yellow solid. MS m/z: 522 [M+H]⁺.

Step 2: 1-(2-(difluoromethoxy)benzyl)-3-(piperidin-4-yl)pyrazino[2,3-b]pyrazin-2(1H)-one: Followed general procedure Q using benzyl 4-(4-(2-(difluoromethoxy)benzyl)-3-oxo-3,4-dihydropyrazino[2,3-b]pyrazin-2-yl)piperidine-1-carboxylate (100 mg, 0.191 mmol, 1 equiv) as the starting material to give the crude product 1-(2-(difluoromethoxy)benzyl)-3-(piperidin-4-yl)pyrazino[2,3-b]pyrazin-2(1H)-one (60 mg) as a yellow oil. MS m/z: 390 [M+H]⁺.

Step 3: 1-(2-(difluoromethoxy)benzyl)-3-(1-(2-fluoro-6-methylphenyl)piperidin-4-yl)pyrazino[2,3-b]pyrazin-2(1H)-one: Followed general procedure G using 1-(2-(difluoromethoxy)benzyl)-3-(piperidin-4-yl)pyrazino[2,3-b]pyrazin-2(1H)-one (60 mg, 0.155 mmol, 1 equiv) and 2-bromo-1-fluoro-3-methylbenzene (43.6 mg, 0.232 mmol, 1.5 equiv) as the starting materials to give 1-(2-(difluoromethoxy)benzyl)-3-(1-(2-fluoro-6-methylphenyl)piperidin-4-yl)pyrazino[2,3-b]pyrazin-2(1H)-one (11.3 mg, 14.7%) as a yellow solid. ¹H NMR (400 MHz, Methanol-d₄) δ 8.56 (s, 2H), 7.32-7.26 (m, 1H), 7.21-7.16 (m, 1H), 7.12-7.04 (m, 2H), 7.04-6.98 (m, 2H), 6.92-6.72 (m, 2H), 5.73 (s, 2H), 3.55-3.45 (m, 1H), 3.36-3.32 (m, 1H), 3.30-3.26 (m, 1H), 3.15-3.07 (m, 2H), 2.36 (s, 3H), 2.10-2.02 (m, 4H). MS m/z: 496.15 [M+H]⁺.

### 6-(1-(2-Fluoro-6-methylphenyl)piperidin-4-yl)-2-methyl-8-((3-(trifluoromethyl)pyrazin-2-yl)methyl)pyrido[2,3-d]pyrimidin-7(8H)-one (47)

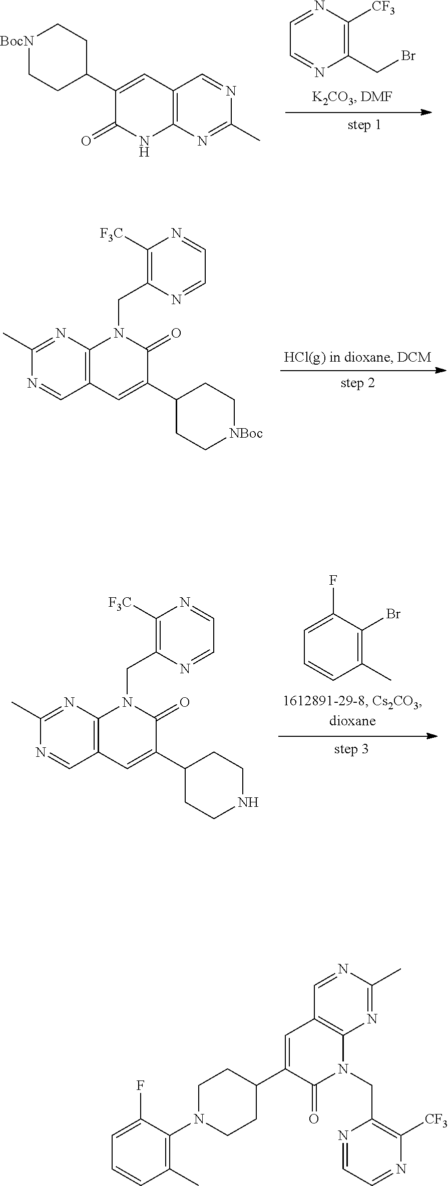

Step 1: tert-butyl 4-(2-methyl-7-oxo-8-((3-(trifluoromethyl)pyrazin-2-yl)methyl)-7,8-dihydropyrido[2,3-d]pyrimidin-6-yl)piperidine-1-carboxylate: Followed general procedure E using tert-butyl 4-(2-methyl-7-oxo-7,8-dihydropyrido[2,3-d]pyrimidin-6-yl)piperidine-1-carboxylate (80 mg, 0.232 mmol, 1 equiv) and 2-(bromomethyl)-3-(trifluoromethyl)pyrazine (61.5 mg, 0.255 mmol, 1.1 equiv) as the starting materials to give tert-butyl 4-(2-methyl-7-oxo-8-((3-(trifluoromethyl)pyrazin-2-yl)methyl)-7,8-dihydropyrido[2,3-d]pyrimidin-6-yl)piperidine-1-carboxylate (60 mg, 51%) as a white solid. MS m/z: 505 [M+H]⁺.

Step 2: 2-methyl-6-(piperidin-4-yl)-8-((3-(trifluoromethyl)pyrazin-2-yl)methyl)pyrido[2,3-d]pyrimidin-7(8H)-one: Followed general procedure F using tert-butyl 4-(2-methyl-7-oxo-8-((3-(trifluoromethyl)pyrazin-2-yl)methyl)-7,8-dihydropyrido[2,3-d]pyrimidin-6-yl)piperidine-1-carboxylate (60 mg, 0.119 mmol, 1 equiv) as the starting material to give the crude product 2-methyl-6-(piperidin-4-yl)-8-((3-(trifluoromethyl)pyrazin-2-yl)methyl)pyrido[2,3-d]pyrimidin-7(8H)-one (50 mg) as an off-white solid. MS m/z: 435 [M+H]⁺.

Step 3: 6-(1-(2-fluoro-6-methylphenyl)piperidin-4-yl)-2-methyl-8-((3-(trifluoromethyl)pyrazin-2-yl)methyl)pyrido[2,3-d]pyrimidin-7(8H)-one: Followed general procedure G using 2-methyl-6-(piperidin-4-yl)-8-((3-(trifluoromethyl)pyrazin-2-yl)methyl)pyrido[2,3-d]pyrimidin-7(8H)-one (50 mg, 0.124 mmol, 1 equiv) and 2-bromo-1-fluoro-3-methylbenzene (35 mg, 0.186 mmol, 1.5 equiv) as the starting materials to give 6-(1-(2-fluoro-6-methylphenyl)piperidin-4-yl)-2-methyl-8-((3-(trifluoromethyl)pyrazin-2-yl)methyl)pyrido[2,3-d]pyrimidin-7(8H)-one (13.3 mg, 20.8%) as a white solid. ¹H NMR (300 MHz, Chloroform-d) δ 8.81 (s, 1H), 8.50 (dd, 2H), 7.62 (s, 1H), 7.03-6.80 (m, 3H), 6.05 (s, 2H), 3.39-3.23 (m, 2H), 3.16-3.00 (m, 3H), 2.64 (s, 3H), 2.35 (s, 3H), 2.09-1.93 (m, 2H), 1.85-1.73 (m, 2H). MS m/z: 513.15 [M+H]⁺.

### 7-(1-(2-Fluoro-6-methylphenyl)azetidin-3-yl)-5-((3-(trifluoromethyl)pyridin-2-yl)methyl)pyrido[2,3-b]pyrazin-6(5H)-one (48)

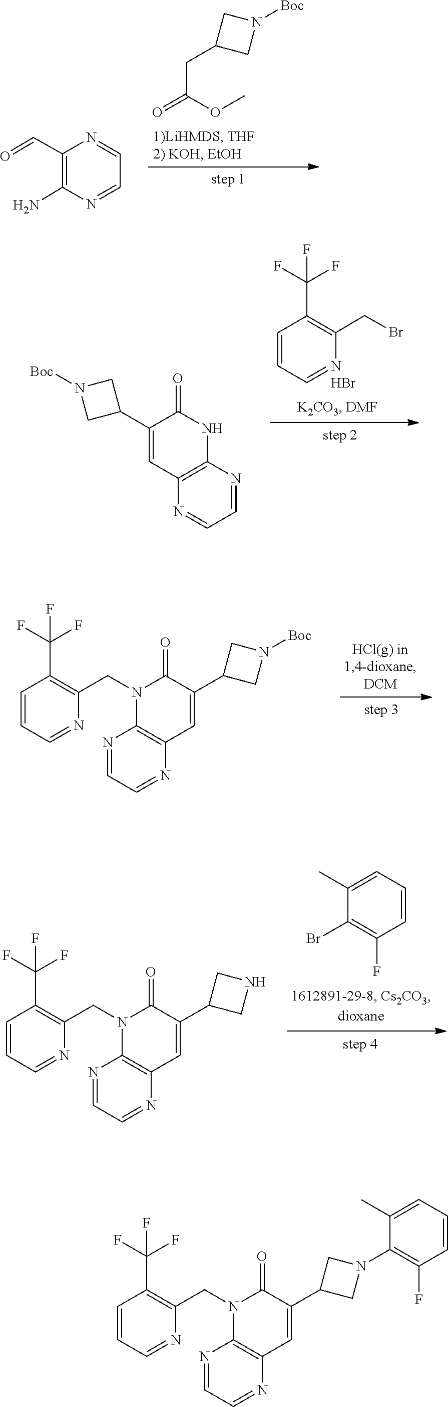

Step 1: tert-butyl 3-(6-oxo-5,6-dihydropyrido[2,3-b]pyrazin-7-yl)azetidine-1-carboxylate: Followed general procedure D using tert-butyl 4-(2-ethoxy-2-oxoethyl)piperazine-1-carboxylate (234 mg, 0.728 mmol, 1.2 equiv) and 3-aminopyrazine-2-carbaldehyde (150 mg, 1.22 mmol, 1 equiv) as the starting materials to give tert-butyl 3-(6-oxo-5,6-dihydropyrido[2,3-b]pyrazin-7-yl)azetidine-1-carboxylate (230 mg, 62%) as a pink solid. MS m/z: 303 [M+H]⁺.

Step 2: tert-butyl 3-(6-oxo-5-((3-(trifluoromethyl)pyridin-2-yl)methyl)-5,6-dihydropyrido[2,3-b]pyrazin-7-yl)azetidine-1-carboxylate: Followed general procedure F using 2-(bromomethyl)-3-(trifluoromethyl)pyridine hydrobromide (234 mg, 0.728 mmol, 1 equiv) as the starting material to give tert-butyl 3-(6-oxo-5-((3-(trifluoromethyl)pyridin-2-yl)methyl)-5,6-dihydropyrido[2,3-b]pyrazin-7-yl)azetidine-1-carboxylate (120 mg, 65%) as a white solid. MS m/z: 462[M+H]⁺.

Step 3: 7-(azetidin-3-yl)-5-((3-(trifluoromethyl)pyridin-2-yl)methyl)pyrido[2,3-b]pyrazin-6(5H)-one: Followed general procedure F using tert-butyl 3-(6-oxo-5-((3-(trifluoromethyl)pyridin-2-yl)methyl)-5,6-dihydropyrido[2,3-b]pyrazin-7-yl)azetidine-1-carboxylate (120 mg, 0.26 mmol, 1 equiv) as the starting material to give the crude product 7-(azetidin-3-yl)-5-((3-(trifluoromethyl)pyridin-2-yl)methyl)pyrido[2,3-b]pyrazin-6(5H)-one (80 mg) as a white solid. MS m/z: 362 [M+H]⁺.

Step 4: 7-(1-(2-fluoro-6-methylphenyl)azetidin-3-yl)-5-((3-(trifluoromethyl)pyridin-2-yl)methyl)pyrido[2,3-b]pyrazin-6(5H)-one: Followed general procedure G using 7-(azetidin-3-yl)-5-((3-(trifluoromethyl)pyridin-2-yl)methyl)pyrido[2,3-b]pyrazin-6(5H)-one (80 mg, 0.194 mmol, 1 equiv) and 2-bromo-1-fluoro-3-methylbenzene (43.9 mg, 0.233 mmol, 1.2 equiv) as the starting materials to give 7-(1-(2-fluoro-6-methylphenyl)azetidin-3-yl)-5-((3-(trifluoromethyl)pyridin-2-yl)methyl)pyrido[2,3-b]pyrazin-6(5H)-one (44.9 mg, 48.7%) as a yellow solid. ¹H NMR (400 MHz, Chloroform-d) δ 8.48 (d, 1H), 8.37 (dd, 2H), 8.06 (d, 1H), 7.96 (d, 1H), 7.24-7.22 (m, 1H), 6.86-6.75 (m, 2H), 6.65 (m, 1H), 6.03 (s, 2H), 4.66 (m, 2H), 4.26 (td, 2H), 4.15-4.03 (m, 1H), 2.28 (s, 3H), 1.26 (s, 1H). MS m/z: 470.15 [M+H]⁺.

### (R)-7-(1-(2-Fluoro-6-methylphenyl)piperidin-4-yl)-5-(5,6,7,8-tetrahydroquinolin-5-yl)pyrido[2,3-b]pyrazin-6(5H)-one (49a); (S)-7-(1-(2-Fluoro-6-methylphenyl)piperidin-4-yl)-5-(5,6,7,8-tetrahydroquinolin-5-yl)pyrido[2,3-b]pyrazin-6(5H)-one (49b)

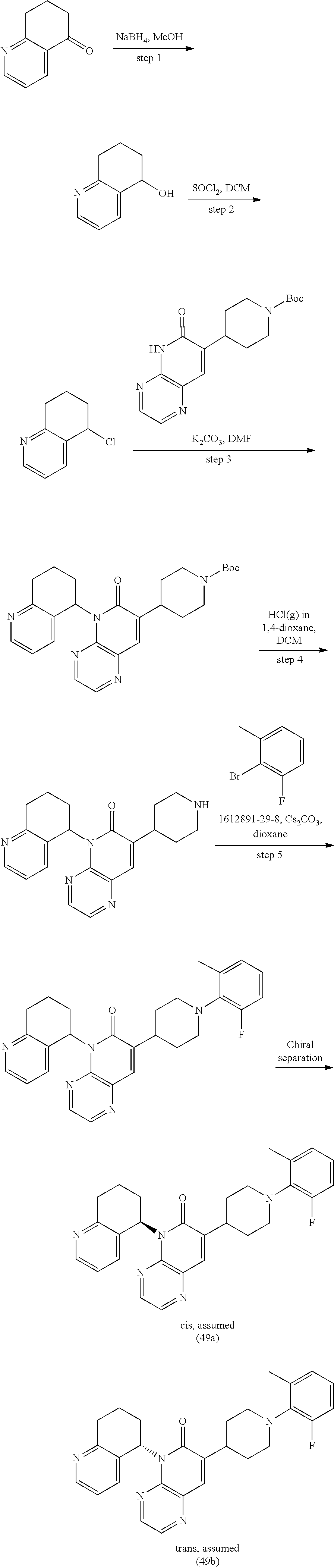

Step 1: 5,6,7,8-tetrahydroquinolin-5-ol: Followed general procedure I using 7,8-dihydroquinolin-5(6H)-one (500 mg, 3.39 mmol, 1 equiv) as the starting material to give 5,6,7,8-tetrahydroquinolin-5-ol (500 mg, 98%) as a yellow oil. MS m/z: 150[M+H]⁺.

Step 2: 5-chloro-5,6,7,8-tetrahydroquinoline: Followed general procedure J using 5,6,7,8-tetrahydroquinolin-5-ol (470 mg, 3.15 mmol, 1 equiv) as the starting material to give the crude product 5-chloro-5,6,7,8-tetrahydroquinoline (350 mg) as a yellow solid. MS m/z: 169 [M+H]⁺.

Step 3: tert-butyl 4-(6-oxo-5-(5,6,7,8-tetrahydroquinolin-5-yl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (500 mg, 1.51 mmol, 1 equiv) and 5-chloro-5,6,7,8-tetrahydroquinoline (279 mg, 1.66 mmol, 1.1 equiv) as the starting materials to give the crude product tert-butyl 4-(6-oxo-5-(5,6,7,8-tetrahydroquinolin-5-yl)-5,6-dihydropyrido[2,3-b]pyrazin-7-yl)piperidine-1-carboxylate (470 mg, 67%) as a yellow oil. MS m/z: 462 [M+H]⁺.

Step 4: 7-(piperidin-4-yl)-5-(5,6,7,8-tetrahydroquinolin-5-yl)pyrido[2,3-b]pyrazin-6(5H)-one: Followed general procedure F using tert-butyl 4-(6-oxo-5-(5,6,7,8-tetrahydroquinolin-5-yl)-5,6-dihydropyrido[2,3-b]pyrazin-7-yl)piperidine-1-carboxylate (470 mg, 1.01 mmol, 1 equiv) as the starting material to give the crude product 7-(piperidin-4-yl)-5-(5,6,7,8-tetrahydroquinolin-5-yl)pyrido[2,3-b]pyrazin-6(5H)-one (230 mg) as a yellow oil. MS m/z: 362 [M+H]⁺.

Step 5: 7-(1-(2-fluoro-6-methylphenyl)piperidin-4-yl)-5-(5,6,7,8-tetrahydroquinolin-5-yl)pyrido[2,3-b]pyrazin-6(5H)-one: Followed general procedure G using 77-(piperidin-4-yl)-5-(5,6,7,8-tetrahydroquinolin-5-yl)pyrido[2,3-b]pyrazin-6(5H)-one (120 mg, 0.332 mmol, 1 equiv) and 2-bromo-1-fluoro-3-methylbenzene (94.1 mg, 0.498 mmol, 1.5 equiv) as the starting materials to give the crude product 7-(1-(2-fluoro-6-methylphenyl)piperidin-4-yl)-5-(5,6,7,8-tetrahydroquinolin-5-yl)pyrido[2,3-b]pyrazin-6(5H)-one (120 mg) as an off-white soil. The crude product was purified by Prep-HPLC with the following conditions (Column: CHIRALPAK IA, 2*25 cm, 5 m; Mobile Phase A: Hex (10 mM NH₃-MeOH), Mobile Phase B: EtOH-HPLC; Flow rate: 20 mL/min; Gradient: 30% B to 30% B in 10 min; Wave Length: 220/203 nm; RT1 (min): 5.4725; RT2 (min): 7.21; Sample Solvent: MeOH:DCM=1:1-HPLC; Injection Volume: 0.45 mL; Number Of Runs: 5) to afford (R)-7-(1-(2-fluoro-6-methylphenyl)piperidin-4-yl)-5-(5,6,7,8-tetrahydroquinolin-5-yl)pyrido[2,3-b]pyrazin-6(5H)-one (49a, 20.2 mg, 16.8%) as an off-white solid and (S)-7-(1-(2-fluoro-6-methylphenyl)piperidin-4-yl)-5-(5,6,7,8-tetrahydroquinolin-5-yl)pyrido[2,3-b]pyrazin-6(5H)-one (49b, 35.7 mg, 39.7%) as an off-white solid.

(R)-7-(1-(2-fluoro-6-methylphenyl)piperidin-4-yl)-5-(5,6,7,8-tetrahydroquinolin-5-yl)pyrido[2,3-b]pyrazin-6(5H)-one (49a)¹H NMR (400 MHz, Chloroform-d) δ 8.52 (d, 1H), 8.40-8.08 (m, 2H), 7.83 (s, 1H), 7.09-6.80 (m, 6H), 3.40-2.99 (m, 7H), 2.74-2.58 (m, 1H), 2.35 (s, 3H), 2.29-2.22 (m, 1H), 2.21-1.82 (m, 6H). MS m/z: 470.10 [M+H]⁺.

(S)-7-(1-(2-fluoro-6-methylphenyl)piperidin-4-yl)-5-(5,6,7,8-tetrahydroquinolin-5-yl)pyrido[2,3-b]pyrazin-6(5H)-one (49b)¹H NMR (400 MHz, Chloroform-d) δ 8.52 (d, 1H), 8.40-8.06 (m, 2H), 7.83 (s, 1H), 7.10-6.81 (m, 6H), 3.39-3.01 (m, 7H), 2.73-2.61 (m, 1H), 2.35 (s, 3H), 2.30-2.23 (m, 1H), 2.21-1.94 (m, 5H), 1.84 (s, 1H). MS m/z: 470.10 [M+H]⁺

### 3-(1-(2-Fluoro-6-methylphenyl)piperidin-4-yl)-7-methyl-1-((3-(trifluoromethyl)pyridin-2-yl)methyl)pteridine-2,4(1H,3H)-dione (50)

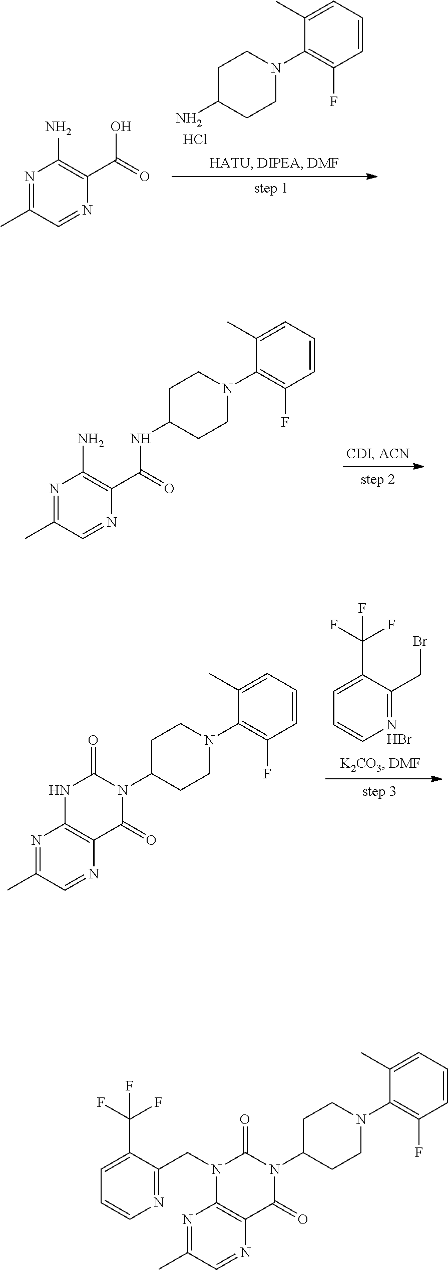

Step 1: 3-amino-N-(1-(2-fluoro-6-methylphenyl)piperidin-4-yl)-5-methylpyrazine-2-carboxamide: Followed general procedure A using 3-amino-5-methylpyrazine-2-carboxylic acid (200 mg, 1.306 mmol, 1 equiv) and 1-(2-fluoro-6-methylphenyl)piperidin-4-amine hydrochloride (351 mg, 1.437 mmol, 1.1 equiv) as the starting materials to give 3-amino-N-(1-(2-fluoro-6-methylphenyl)piperidin-4-yl)-5-methylpyrazine-2-carboxamide (290 mg, 64%) as a yellow solid. MS m/z: 344 [M+H]⁺.

Step 2: 3-(1-(2-fluoro-6-methylphenyl)piperidin-4-yl)-7-methylpteridine-2,4(1H,3H)-dione: Followed general procedure L using 3-amino-N-(1-(2-fluoro-6-methylphenyl)piperidin-4-yl)-5-methylpyrazine-2-carboxamide (150 mg, 0.437 mmol, 1 equiv) as the starting material to give the crude product 3-(1-(2-fluoro-6-methylphenyl)piperidin-4-yl)-7-methylpteridine-2,4(1H,3H)-dione (40 mg) as a white solid. MS m/z: 390 [M+H]⁺.

Step 3: 3-(1-(2-fluoro-6-methylphenyl)piperidin-4-yl)-7-methyl-1-((3-(trifluoromethyl)pyridin-2-yl)methyl)pteridine-2,4(1H,3H)-dione: Followed general procedure G using 3-(1-(2-fluoro-6-methylphenyl)piperidin-4-yl)-7-methylpteridine-2,4(1H,3H)-dione (40 mg, 0.108 mmol, 1 equiv) and 2-(bromomethyl)-3-(trifluoromethyl)pyridine hydrobromide (38.2 mg, 0.141 mmol, 1.1 equiv) as the starting materials to give 3-(1-(2-fluoro-6-methylphenyl)piperidin-4-yl)-7-methyl-1-((3-(trifluoromethyl)pyridin-2-yl)methyl)pteridine-2,4(1H,3H)-dione (22.9 mg, 39.5%) as a white solid. ¹H NMR (400 MHz, Methanol-d₄) δ 8.55-8.47 (m, 1H), 8.44 (s, 1H), 8.20-8.13 (m, 1H), 7.49-7.41 (m, 1H), 7.04-6.93 (m, 2H), 6.91-6.79 (m, 1H), 5.85 (s, 2H), 5.13-5.00 (m, 1H), 3.27-3.23 (m, 1H), 3.11-3.02 (m, 2H), 2.99-2.84 (m, 2H), 2.51 (s, 3H), 2.36 (s, 3H), 2.03 (s, 1H), 1.78-1.69 (m, 2H). MS m/z: 529.25 [M+H]⁺.

### 2-Ethoxy-6-(1-(2-fluoro-6-methylphenyl)piperidin-4-yl)-8-((3-(trifluoromethyl)pyrazin-2-yl)methyl)pyrido[2,3-d]pyrimidin-7(8H)-one (51)

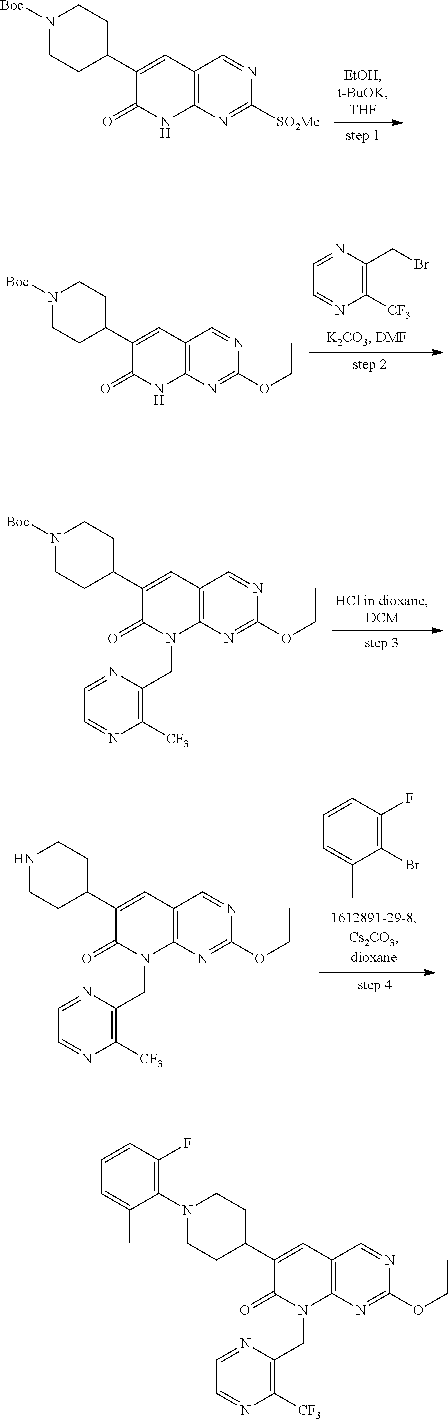

Step 1: tert-butyl 4-(2-ethoxy-7-oxo-7,8-dihydropyrido[2,3-d]pyrimidin-6-yl)piperidine-1-carboxylate: Followed general procedure S using EtOH (50.7 mg, 1.10 mmol, 3 equiv) and tert-butyl 4-(2-(methylsulfonyl)-7-oxo-7,8-dihydropyrido[2,3-d]pyrimidin-6-yl)piperidine-1-carboxy-late (150 mg, 0.367 mmol, 1 equiv) as the starting materials to give tert-butyl 4-(2-ethoxy-7-oxo-7,8-dihydropyrido[2,3-d]pyrimidin-6-yl)piperidine-1-carboxylate (90 mg, 65%) as a white solid. MS m/z: 375 [M+H]⁺.

Step 2: tert-butyl 4-(2-ethoxy-7-oxo-8-((3-(trifluoromethyl)pyrazin-2-yl)methyl)-7,8-dihydropyrido[2,3-d]pyrimidin-6-yl)piperidine-1-carboxylate: Followed general procedure E using tert-butyl 4-(2-ethoxy-7-oxo-7,8-dihydropyrido[2,3-d]pyrimidin-6-yl)piperidine-1-carboxylate (90 mg, 0.240 mmol, 1 equiv) and 2-(bromomethyl)-3-(trifluoromethyl)pyrazine (63.7 mg, 0.264 mmol, 1.1 equiv) as the starting materials to give tert-butyl 4-(2-ethoxy-7-oxo-8-((3-(trifluoromethyl)pyrazin-2-yl)methyl)-7,8-dihydropyrido[2,3-d]-pyrimi-din-6-yl)piperidine-1-carboxylate (60 mg, 46%) as an off-white solid. MS m/z: 535 [M+H]⁺.

Step 3: 2-ethoxy-6-(piperidin-4-yl)-8-((3-(trifluoromethyl)pyrazin-2-yl)methyl)pyrido[2,3-d]pyrimidin-7(8H)-one: Followed general procedure F using tert-butyl 4-(2-ethoxy-7-oxo-8-((3-(trifluoromethyl)pyrazin-2-yl)methyl)-7,8-dihydropyrido[2,3-d]pyrimidin-6-yl)piperidine-1-carboxylate (60 mg, 0.112 mmol, 1 equiv) as the starting material to give the crude product 2-ethoxy-6-(piperidin-4-yl)-8-((3-(trifluoromethyl)pyrazin-2-yl) methyl) pyrido[2,3-d]pyrimidin-7(8H)-one (50 mg) as a yellow oil. MS m/z: 435 [M+H]⁺.

Step 4: 2-ethoxy-6-(1-(2-fluoro-6-methylphenyl)piperidin-4-yl)-8-((3-(trifluoromethyl) pyrazin-2-yl)methyl)pyrido[2,3-d]pyrimidin-7(8H)-one: Followed general procedure G using 2-ethoxy-6-(piperidin-4-yl)-8-((3-(trifluoromethyl)pyrazin-2-yl)methyl)pyrido[2,3-d]pyrimidin-7(8H)-one (50 mg, 0.106 mmol, 1 equiv) and 2-bromo-1-fluoro-3-methylbenzene (22 mg, 0.117 mmol, 1.1 equiv) as the starting materials to give 2-ethoxy-6-(1-(2-fluoro-6-methylphenyl)piperidin-4-yl)-8-((3-(trifluoromethyl)pyrazin-2-yl)methyl)pyrido[2,3-d]pyri-midin-7(8H)-one (18.9 mg, 32.8%) as a white solid. ¹H NMR (300 MHz, Methanol-d₄) δ 8.85 (s, 1H), 8.67-8.54 (m, 2H), 7.92 (d, 1H), 7.08-6.96 (m, 2H), 6.93-6.82 (m, 1H), 6.02 (s, 2H), 4.33-4.19 (m, 2H), 3.32-3.24 (m, 2H), 3.15-3.04 (m, 2H), 3.03-2.94 (m, 1H), 2.38 (s, 3H), 2.07-1.94 (m, 2H), 1.93-1.74 (m, 2H), 1.21 (t, 3H). MS m/z: 543.20 [M+H]⁺.

### 7-(1-(5-Fluoro-2-methylpyridin-3-yl)piperidin-4-yl)-3-methyl-5-((3-(trifluoromethyl)pyrazin-2-yl)methyl)pyrido[2,3-b]pyrazin-6(5H)-one (52)

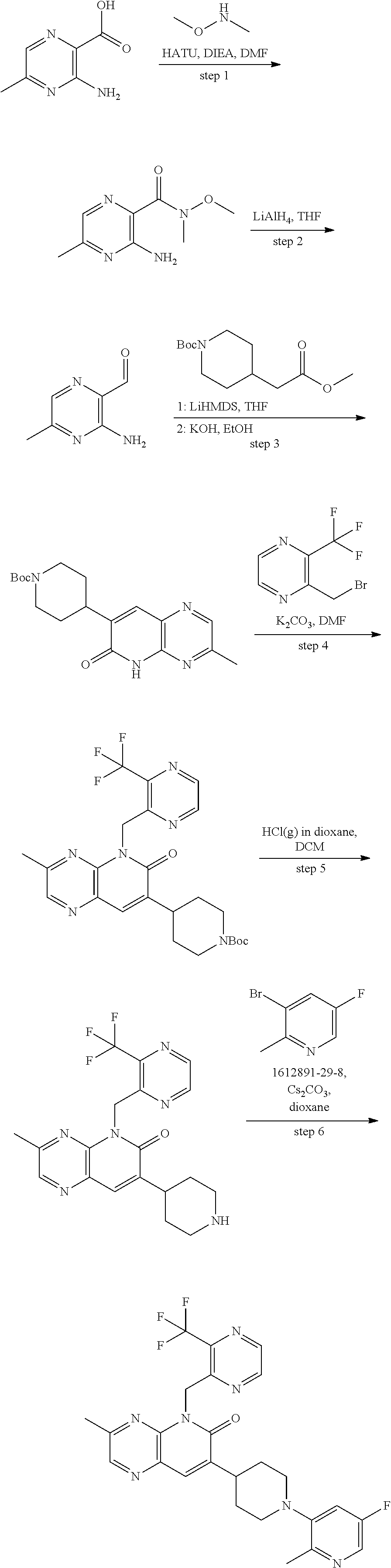

Step 1: 3-amino-N-methoxy-N,5-dimethylpyrazine-2-carboxamide: Followed general procedure A using 3-amino-5-methylpyrazine-2-carboxylic acid (300 mg, 1.95 mmol, 1 equiv) and N,O-dimethylhydroxylamine (179 mg, 2.93 mmol, 1.5 equiv) as the starting materials to give 3-amino-N-methoxy-N,5-dimethylpyrazine-2-carboxamide (320 mg, 83%) as an off-white solid. MS m/z: 197 [M+H]⁺.

Step 2: 3-amino-5-methylpyrazine-2-carbaldehyde: Followed general procedure B using 3-amino-N-methoxy-N,5-dimethylpyrazine-2-carboxamide (320 mg, 1.63 mmol, 1 equiv) as the starting material to give 3-amino-5-methylpyrazine-2-carbaldehyde (200 mg, 89%) as a yellow solid. MS m/z: 138 [M+H]⁺.

Step 3: tert-butyl 4-(3-methyl-6-oxo-5,6-dihydropyrido[2,3-b]pyrazin-7-yl)piperidine-1-carboxylate: Followed general procedure D using tert-butyl 4-(2-methoxy-2-oxoethyl)piperazine-1-carboxylate (452 mg, 1.75 mmol, 1.2 equiv) and 3-amino-5-methylpyrazine-2-carbaldehyde (200 mg, 1.45 mmol, 1 equiv) as the starting materials to give tert-butyl 4-(3-methyl-6-oxo-5,6-dihydropyrido[2,3-b]pyrazin-7-yl)piperidine-1-carboxylate (320 mg, 63%) as a light brown solid. MS m/z: 345 [M+H]⁺.

Step 4: tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (90 mg, 0.261 mmol, 1 equiv) and 2-(bromomethyl)-3-(trifluoromethyl)pyrazine (94.4 mg, 0.392 mmol, 1.5 equiv) as the starting materials to give tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (70 mg, 53%) as an off-white solid. MS m/z: 505 [M+H]⁺.

Step 5: 3-methyl-7-(piperidin-4-yl)-5-((3-(trifluoromethyl)pyrazin-2-yl)methyl)pyrido[2,3-b]pyrazin-6(5H)-one: Followed general procedure F using tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (70 mg, 0.139 mmol, 1 equiv) as the starting material to give the crude product 3-methyl-7-(piperidin-4-yl)-5-((3-(trifluoromethyl)pyrazin-2-yl)methyl)pyrido[2,3-b]pyrazin-6(5H)-one (45 mg) as a off white solid. MS m/z: 405 [M+H]⁺.

Step 6: 7-(1-(5-fluoro-2-methylpyridin-3-yl)piperidin-4-yl)-3-methyl-5-((3-(trifluoromethyl)pyrazin-2-yl)methyl)pyrido[2,3-b]pyrazin-6(5H)-one

Followed general procedure G using 3-methyl-7-(piperidin-4-yl)-5-((3-(trifluoromethyl)pyrazin-2-yl)methyl)pyrido[2,3-b]pyrazin-6(5H)-one (45 mg, 0.111 mmol, 1 equiv) and 3-bromo-5-fluoro-2-methylpyridine (25.3 mg, 0.133 mmol, 1.2 equiv) as the starting materials to give 7-(1-(5-fluoro-2-methylpyridin-3-yl)piperidin-4-yl)-3-methyl-5-((3-(trifluoromethyl)pyrazin-2-yl)methyl)pyrido[2,3-b]pyrazin-6(5H)-one (19.3 mg, 33.4%) as a pink solid. ¹H NMR (400 MHz, Chloroform-d) δ 8.54-8.50 (m, 1H), 8.48-8.44 (m, 1H), 8.37 (s, 1H), 8.09 (d, 1H), 7.87 (s, 1H), 7.12 (d, 1H), 6.08 (s, 2H), 3.30 (d, 2H), 3.20-3.09 (m, 1H), 2.88-2.78 (m, 2H), 2.59-2.50 (m, 6H), 2.15 (d, 2H), 1.94-1.82 (m, 2H). MS m/z: 514.1 [M+H]⁺.

### (R)-7-(1-(2-Fluoro-6-methylphenyl)piperidin-4-yl)-5-(1,2,3,4-tetrahydronaphthalen-1-yl)pyrido[2,3-b]pyrazin-6(5H)-one (53a); (S)-7-(1-(2-Fluoro-6-methylphenyl)piperidin-4-yl)-5-(1,2,3,4-tetrahydronaphthalen-1-yl)pyrido[2,3-b]pyrazin-6(5H)-one (53b)

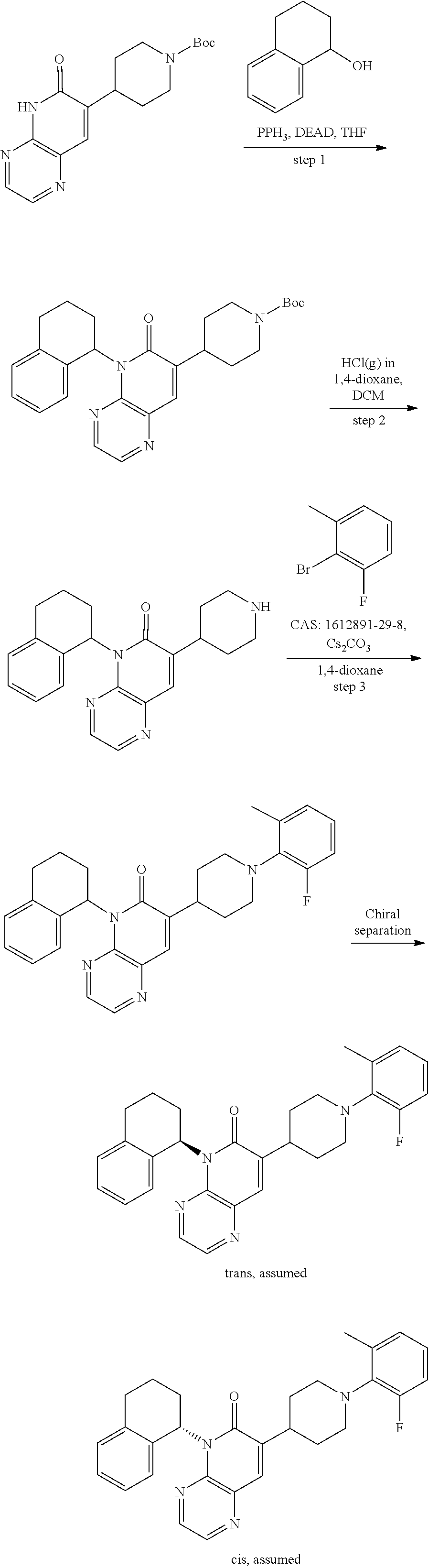

Step 1: tert-butyl4-(6-oxo-5-(1,2,3,4-tetrahydronaphthalen-1-yl)-5,6-dihydropyrido[2,3-b]pyrazin-7-yl) piperidine-1-carboxylate: Followed general procedure X using 1,2,3,4-tetrahydronaphthalen-1-ol (116 mg, 0.786 mmol, 1.3 equiv) and tert-butyl 4-(6-oxo-5,6-dihydropyrido[2,3-b]pyrazin-7-yl)piperidine-1-carboxylate (200 mg, 0.605 mmol, 1 equiv) as the starting materials to give tert-butyl 4-(6-oxo-5-(1,2,3,4-tetrahydronaphthalen-1-yl)-5,6-dihydropyrido[2,3-b]pyrazin-7-yl)piperidine-1-carboxy-late (151 mg, 54%) as a light yellow solid. MS m/z: 461[M+H]⁺.

Step 2: 7-(piperidin-4-yl)-5-(1,2,3,4-tetrahydronaphthalen-1-yl)pyrido[2,3-b]pyrazin-6(5H)-one: Followed general procedure F using tert-butyl 4-(6-oxo-5-(1,2,3,4-tetrahydronaphthalen-1-yl)-5,6-dihydropyrido[2,3-b]pyrazin-7-yl)piperidine-1-carboxylate (151 mg, 0.328 mmol, 1 equiv) as the starting material to give the crude product 7-(piperidin-4-yl)-5-(1,2,3,4-tetrahydronaphthalen-1-yl)pyrido[2,3-b]pyrazin-6(5H)-one (118 mg) as a yellow oil. MS m/z: 361 [M+H]⁺.

Step 3: 7-(1-(2-fluoro-6-methylphenyl)piperidin-4-yl)-5-(1,2,3,4-tetrahydronaphthalen-1-yl) pyrido[2,3-b]pyrazin-6(5H)-one: Followed general procedure G using 7-(piperidin-4-yl)-5-(1,2,3,4-tetrahydronaphthalen-1-yl)pyrido[2,3-b]pyrazin-6(5H)-one (118 mg, 0.327 mmol, 1.00 equiv) and 2-bromo-1-fluoro-3-methylbenzene (74.2 mg, 0.392 mmol, 1.20 equiv) as the starting materials to give the crude product 7-(1-(2-fluoro-6-methylphenyl)piperidin-4-yl)-5-(1,2,3,4-tetrahydronaphthalen-1-yl) pyrido[2,3-b]pyrazin-6(5H)-one (100 mg) as a yellow solid. The crude product was purified by Prep-HPLC with the following conditions (Column: CHIRALPAK IA, 2*25 cm, 5 m; Mobile Phase A: Hex (10 mM NH₃-MeOH), Mobile Phase B: EtOH-HPLC; Flow rate: 20 mL/min; Gradient: 50% B to 50% B in 12 min; Wave Length: 21/342 nm; RT1 (min): 4.295; RT2 (min): 6.24; Sample Solvent: MeOH:DCM=1:1-HPLC; Injection Volume: 0.5 mL; Number Of Runs: 7) to afford (R)-7-(1-(2-fluoro-6-methylphenyl)piperidin-4-yl)-5-(1,2,3,4-tetrahydronaphthalen-1-yl)pyrido[2,3-b]pyrazin-6(5H)-one (53a, 34.1 mg, 22.2%) as a light yellow solid, and (S)-7-(1-(2-fluoro-6-methylphenyl)piperidin-4-yl)-5-(1,2,3,4-tetrahydronaphthalen-1-yl)pyrido[2,3-b]pyrazin-6(5H)-one (53b, 28.3 mg, 18.4%) as a light yellow solid.

(R)-7-(1-(2-fluoro-6-methylphenyl)piperidin-4-yl)-5-(1,2,3,4-tetrahydronaphthalen-1-yl)pyrido[2,3-b]pyrazin-6(5H)-one (53a) LCMS (ES, m/z): 469.2 [M+H]⁺. ¹H NMR (400 MHz, Chloroform-d) δ 8.50-8.03 (m, 2H), 7.84 (s, 1H), 7.15-6.85 (m, 7H), 6.62 (d, H), 3.37 (s, 2H), 3.16-2.96 (m, 4H), 2.85 (d, 1H), 2.72-2.63 (m, 1H), 2.42 (s, 3H), 2.18-1.90 (m, 7H).

(S)-7-(1-(2-fluoro-6-methylphenyl)piperidin-4-yl)-5-(1,2,3,4-tetrahydronaphthalen-1-yl)pyrido[2,3-b]pyrazin-6(5H)-one (53b) LCMS (ES, m/z): 469.2 [M+H]⁺. ¹H NMR (400 MHz, Chloroform-d) δ 8.50-8.03 (m, 2H), 7.84 (s, 1H), 7.15-6.86 (m, 7H), 6.62 (d, 1H), 3.37 (s, 2H), 3.16-2.96 (m, 4H), 2.85 (d, 1H), 2.72-2.63 (m, 1H), 2.42 (s, 3H), 2.18-1.91 (m, 7H).

### 7-(1-(2-Fluoro-6-methylphenyl)piperidin-4-yl)-3-methoxy-5-((3-(trifluoromethyl)pyridin-2-yl)methyl)pyrido[2,3-b]pyrazin-6(5H)-one (54)

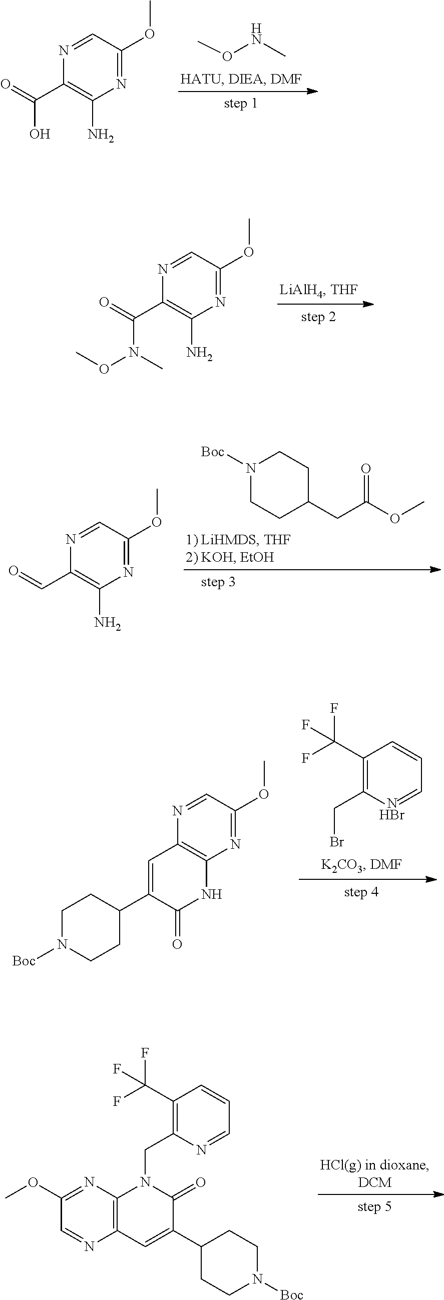

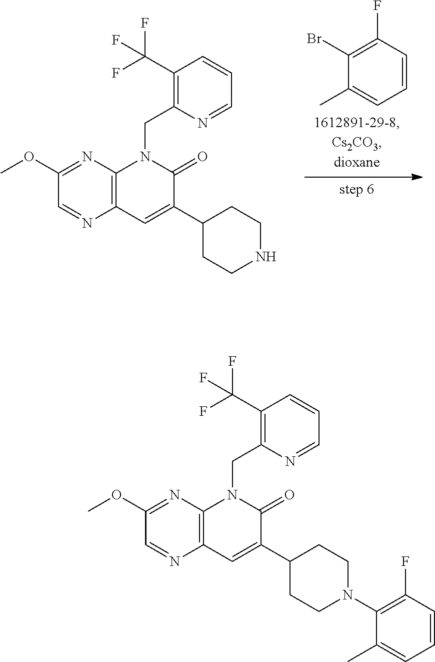

Step 1: 3-amino-N,5-dimethoxy-N-methylpyrazine-2-carboxamide: Followed general procedure A using 3-amino-5-methoxypyrazine-2-carboxylic acid (200 mg, 1.18 mmol, 1 equiv) and N,O-dimethylhydroxylamine (108 mg, 1.77 mmol, 1.5 equiv) as the starting materials to give 3-amino-N,5-dimethoxy-N-methylpyrazine-2-carboxamide (245 mg, 95%) as a yellow solid. MS m/z: 212 [M+H]⁺.

Step 2: 3-amino-5-methoxypyrazine-2-carbaldehyde: Followed general procedure B using 3-amino-N,5-dimethoxy-N-methylpyrazine-2-carboxamide (245 mg, 1.15 mmol, 1 equiv) as the starting material to give 3-amino-5-methoxypyrazine-2-carbaldehyde (132 mg, 74%) as a yellow solid. MS m/z: 154 [M+H]⁺.

Step 3: tert-butyl 4-(3-methoxy-6-oxo-5,6-dihydropyrido[2,3-b]pyrazin-7-yl)piperidine-1-carboxylate: Followed general procedure D using 3-amino-5-methoxypyrazine-2-carbaldehyde (132 mg, 0.862 mmol, 1 equiv) and tert-butyl 4-(2-methoxy-2-oxoethyl)piperidine-1-carboxylate (266 mg, 1.03 mmol, 1.2 equiv) as the starting materials to give tert-butyl 4-(6-oxo-5,6-dihydropyrido[2,3-b]pyrazin-7-yl)piperazine-1-carboxylate (80 mg, 26%) as a yellow solid. MS m/z: 361 [M+H]⁺.

Step 4: tert-butyl 4-(3-methoxy-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(3-methoxy-6-oxo-5,6-dihydropyrido[2,3-b]pyrazin-7-yl)piperidine-1-carboxylate (80 mg, 0.222 mmol, 1 equiv) and 2-(bromomethyl)-3-(trifluoromethyl)pyridine hydrobromide (71.2 mg, 0.222 mmol, 1 equiv) as the starting materials to give tert-butyl 4-(3-methoxy-6-oxo-5-((3-(trifluoromethyl)pyridin-2-yl)methyl)-5,6-dihydropyrido[2,3-b]pyrazin-7-yl)piperidine-1-carboxylate (100 mg, 86%) as a off-white solid. MS m/z: 520[M+H]⁺.

Step 5: 3-methoxy-7-(piperidin-4-yl)-5-((3-(trifluoromethyl)pyridin-2-yl)methyl)pyrido[2,3-b]pyrazin-6(5H)-one: Followed general procedure F using tert-butyl 4-(3-methoxy-6-oxo-5-((3-(trifluoromethyl)pyridin-2-yl)methyl)-5,6-dihydropyrido[2,3-b]pyrazin-7-yl)piperidine-1-carboxylate (100 mg, 0.192 mmol, 1 equiv) as the starting material to give the crude product 3-methoxy-7-(piperidin-4-yl)-5-((3-(trifluoromethyl)pyridin-2-yl)methyl)pyrido[2,3-b]pyrazin-6(5H)-one (80 mg) as a yellow oil. MS m/z: 420 [M+H]⁺.

Step 6: 7-(1-(2-fluoro-6-methylphenyl)piperidin-4-yl)-3-methoxy-5-((3-(trifluoromethyl)pyridin-2-yl)methyl)pyrido[2,3-b]pyrazin-6(5H)-one: Followed general procedure G using 3-methoxy-7-(piperidin-4-yl)-5-((3-(trifluoromethyl)pyridin-2-yl)methyl)pyrido[2,3-b]pyrazin-6(5H)-one (80 mg, 0.191 mmol, 1 equiv) and 2-bromo-1-fluoro-3-methylbenzene (43.2 mg, 0.229 mmol, 1.2 equiv) as the starting materials to give 7-(1-(2-fluoro-6-methylphenyl)piperidin-4-yl)-3-methoxy-5-((3-(trifluoromethyl)pyridin-2-yl)methyl)pyrido[2,3-b]pyrazin-6(5H)-one (35.0 mg, 34.1%) as a white solid. ¹H NMR (400 MHz, Chloroform-d) δ 8.85-8.81 (m, 1H), 8.39 (s, 1H), 8.15 (s, 1H), 8.10-8.05 (m, 1H), 7.47 (dd, 1H), 7.02-6.94 (m, 2H), 6.89-6.79 (m, 1H), 5.93 (s, 2H), 4.17 (s, 3H), 3.29-3.17 (m, 2H), 3.15-3.02 (m, 3H), 2.36 (s, 3H), 2.04-1.90 (m, 4H). MS m/z: 528.2 [M+H]⁺.

### 7-(1-(2-Fluoro-6-methylphenyl)piperidin-4-yl)-2-methyl-5-(2-(trifluoromethyl)-benzyl)-pyrido[2,3-b]pyrazin-6(5H)-one (55)

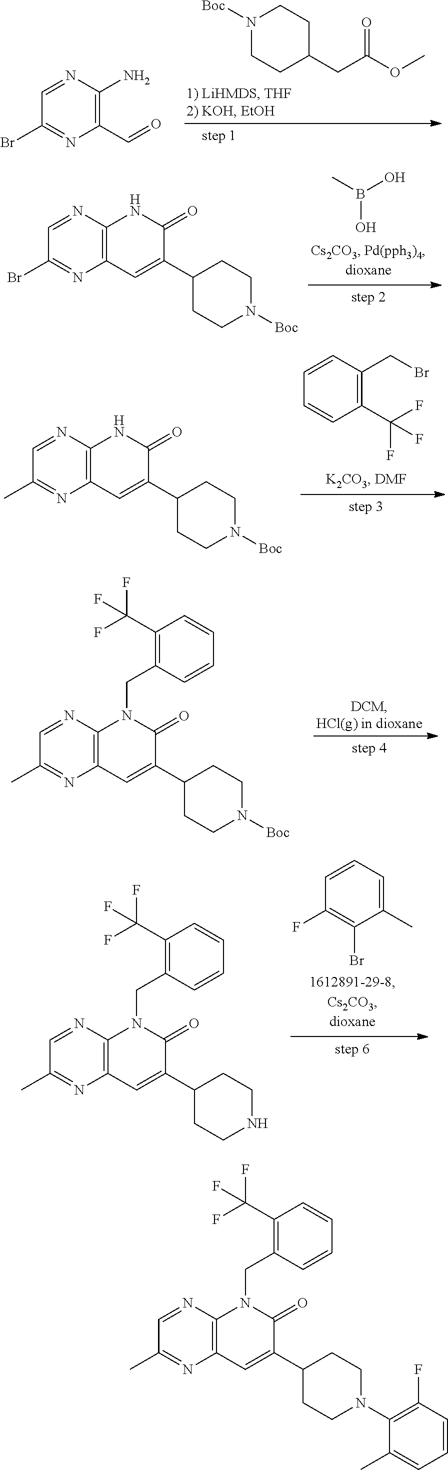

Step 1: tert-butyl 4-(2-bromo-6-oxo-5,6-dihydropyrido[2,3-b]pyrazin-7-yl)piperidine-1-carboxylate: Followed general procedure D using tert-butyl 4-(2-methoxy-2-oxoethyl)piperidine-1-carboxylate (382 mg, 1.48 mmol, 1.5 equiv) and 3-amino-6-bromopyrazine-2-carbaldehyde (200 mg, 0.99 mmol, 1 equiv) as the starting materials to give tert-butyl 4-(2-bromo-6-oxo-5,6-dihydropyrido[2,3-b]pyrazin-7-yl)piperidine-1-carboxylate (45 mg, 11%) as a white solid. MS m/z: 409 [M+H]⁺.

Step 2: tert-butyl 4-(2-methyl-6-oxo-5,6-dihydropyrido[2,3-b]pyrazin-7-yl)piperidine-1-carboxylate: Followed general procedure U using tert-butyl 4-(2-bromo-6-oxo-5,6-dihydropyrido[2,3-b]pyrazin-7-yl)piperidine-1-carboxylate (40 mg, 0.098 mmol, 1 equiv) and methylboronic acid (8.78 mg, 0.147 mmol, 1.5 equiv) as the starting materials to give tert-butyl 4-(2-methyl-6-oxo-5,6-dihydropyrido[2,3-b]pyrazin-7-yl)piperidine-1-carboxylate (30 mg, 89%) as an off-white solid. MS m/z: 345 [M+H]⁺.

Step 3: tert-butyl 4-(2-methyl-6-oxo-5-(2-(trifluoromethyl)benzyl)-5,6-dihydropyrido[2,3-b]pyrazin-7-yl)piperidine-1-carboxylate: Followed general procedure E using tert-butyl 4-(2-methyl-6-oxo-5,6-dihydropyrido[2,3-b]pyrazin-7-yl)piperidine-1-carboxylate (30 mg, 0.087 mmol, 1 equiv) and 1-(bromomethyl)-2-(trifluoromethyl)benzene (22.9 mg, 0.096 mmol, 1.1 equiv) as the starting materials to give tert-butyl 4-(2-methyl-6-oxo-5-(2-(trifluoromethyl)benzyl)-5,6-dihydropyrido[2,3-b]pyrazin-7-yl)-piperi-dine-1-carboxylate (30 mg, 68%) as a yellow solid. MS m/z: 503 [M+H]⁺.

Step 4: 2-methyl-7-(piperidin-4-yl)-5-(2-(trifluoromethyl)benzyl)pyrido[2,3-b]pyrazin-6(5H)-one hydrochloride: Followed general procedure F using tert-butyl 4-(2-methyl-6-oxo-5-(2-(trifluoromethyl)benzyl)-5,6-dihydropyrido[2,3-b]pyrazin-7-yl)piperidine-1-carboxylate (30 mg, 0.06 mmol, 1 equiv) as the starting material to give the crude product 2-methyl-7-(piperidin-4-yl)-5-(2-(trifluoromethyl)benzyl)pyrido[2,3-b]pyrazin-6(5H)-one (20 mg) as a yellow oil. MS m/z: 439 [M+H]⁺.

Step 5: 7-(1-(2-fluoro-6-methylphenyl)piperidin-4-yl)-2-methyl-5-(2-(trifluoromethyl)-benzyl)pyrido[2,3-b]pyrazin-6(5H)-one: Followed general procedure G using 2-methyl-7-(piperidin-4-yl)-5-(2-(trifluoromethyl)benzyl)-pyrido[2,3-b]pyrazin-6(5H)-one (20 mg, 0.05 mmol, 1 equiv) and 2-bromo-1-fluoro-3-methylbenzene (10.3 mg, 0.055 mmol, 1.1 equiv) as the starting materials to give 7-(1-(2-fluoro-6-methylphenyl)piperidin-4-yl)-2-methyl-5-(2-(trifluoromethyl)benzyl)pyrido-[2,3-b]pyrazin-6(5H)-one (5.9 mg, 22.9%) as an off-white solid. ¹H NMR (300 MHz, Chloroform-d) δ 8.31 (s, 1H), 7.92 (s, 1H), 7.79-7.64 (m, 1H), 7.39-7.30 (m, 2H), 7.10-6.95 (m, 2H), 6.95-6.82 (m, 1H), 6.72-6.58 (m, 1H), 5.96 (s, 2H), 3.49-3.32 (m, 2H), 3.31-3.22 (m, 1H), 3.22-3.09 (m, 2H), 2.66 (s, 3H), 2.42 (s, 3H), 2.17-2.00 (m, 2H), 1.99-1.81 (m, 2H). MS m/z: 511.15 [M+H]⁺.

### 7-(1-(5-Fluoro-2-methylpyridin-3-yl)piperidin-4-yl)-3-methyl-5-((3-(trifluoromethyl)py-ridin-2-yl)methyl)pyrido[2,3-b]pyrazin-6(5H)-one (56)

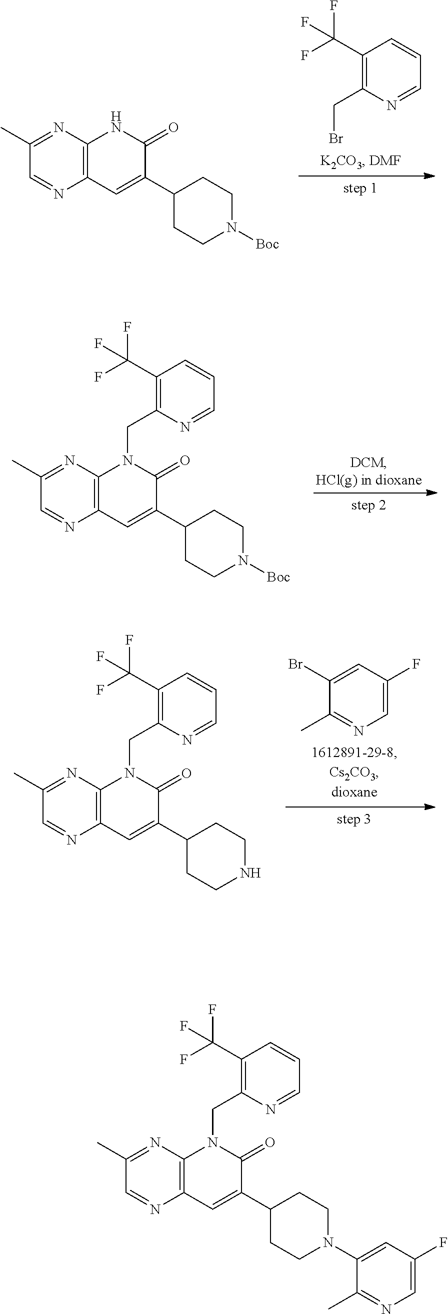

Step 1: tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.29 mmol, 1 equiv) and 2-(bromomethyl)-3-(trifluoromethyl)pyridine (76.6 mg, 0.319 mmol, 1.1 equiv) as the starting materials to give tert-butyl 4-(3-methyl-6-oxo-5-((3-(trifluoromethyl)pyridin-2-yl)methyl)-5,6-dihydropyrido[2,3-b]pyra-zin-7-yl)piperidine-1-carboxylate (40 mg, 27%) as a yellow solid. MS m/z: 504 [M+H]⁺.

Step 2: 3-methyl-7-(piperidin-4-yl)-5-((3-(trifluoromethyl)pyridin-2-yl)methyl)pyrido[2,3-b]pyrazin-6(5H)-one hydrochloride: Followed general procedure F using tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (40 mg, 0.079 mmol, 1 equiv) as the starting material to give the crude product 3-methyl-7-(piperidin-4-yl)-5-((3-(trifluoromethyl)pyridin-2-yl)methyl)pyrido[2,3-b]pyrazin-6(5H)-one (30 mg) as an off-white solid. MS m/z: 404 [M+H]⁺.

Step 3: 7-(1-(5-fluoro-2-methylpyridin-3-yl)piperidin-4-yl)-3-methyl-5-((3-(trifluoromethyl)-pyridin-2-yl)methyl)pyrido[2,3-b]pyrazin-6(5H)-one: Followed general procedure G using 3-methyl-7-(piperidin-4-yl)-5-((3-(trifluoromethyl)pyridin-2-yl)methyl)pyrido[2,3-b]pyrazin-6(5H)-one hydrochloride (30 mg, 0.074 mmol, 1 equiv) and 3-bromo-5-fluoro-2-methylpyridine (15.5 mg, 0.081 mmol, 1.1 equiv) as the starting materials to give 7-(1-(5-fluoro-2-methylpyridin-3-yl)piperidin-4-yl)-3-methyl-5-((3-(trifluoromethyl)pyridin-2-yl)methyl)pyri-do[2,3-b]pyra-zin-6(5H)-one (17.0 mg, 44.1%) as a light yellow solid. ¹H NMR (300 MHz, Chloroform-d) δ 8.46-8.38 (m, 1H), 8.37-8.29 (m, 1H), 8.08 (d, 1H), 8.01-7.92 (m, 1H), 7.88-7.78 (m, 1H), 7.25-7.20 (m, 1H), 7.15-7.01 (m, 1H), 6.03 (s, 2H), 3.34-3.23 (m, 2H), 3.22-3.08 (m, 1H), 2.81 (t, 2H), 2.58-2.47 (m, 6H), 2.21-2.05 (m, 2H), 1.96-1.77 (m, 1H). MS m/z: 513.20 [M+H]⁺.

### 7-(1-(2-Fluoro-6-methylphenyl)piperidin-4-yl)-8-methyl-5-((3-(trifluoromethyl)pyridin-2-yl)methyl)pyrido[2,3-b]pyrazin-6(5H)-one (57)

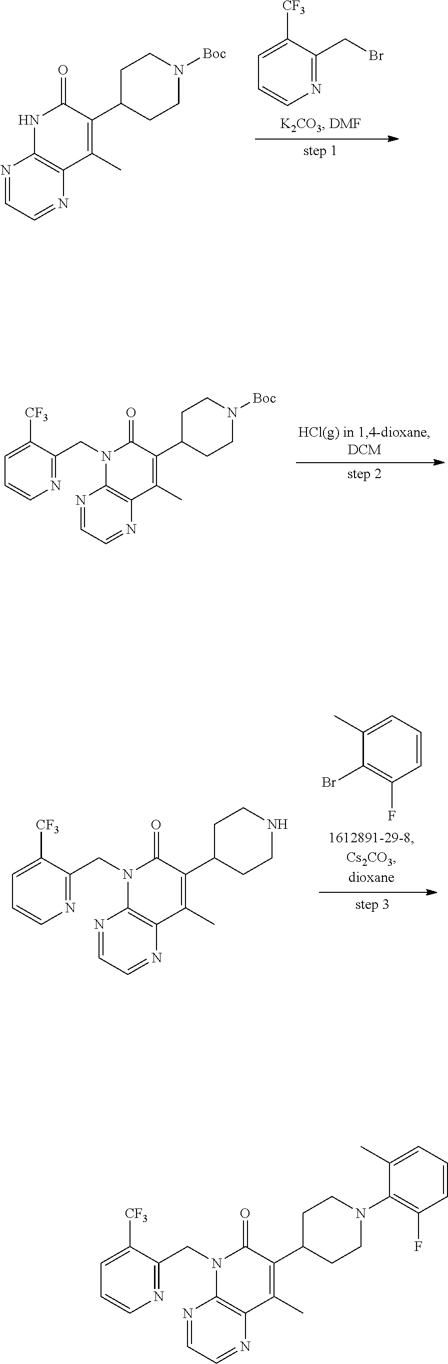

Step 1: tert-butyl 4-(8-methyl-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (100 mg, 0.29 mmol, 1 equiv) and 2-(bromomethyl)-3-(trifluoromethyl)pyridine hydrobromide (83.9 mg, 0.261 mmol, 0.9 equiv) as the starting materials to give tert-butyl 4-(8-methyl-6-oxo-5-((3-(trifluoromethyl)pyridin-2-yl)methyl)-5,6-dihydropyrido[2,3-b]pyrazin-7-yl)piperidine-1-carboxylate (80 mg, 54%) as a yellow oil. MS m/z: 504 [M+H]⁺.

Step 2: 8-methyl-7-(piperidin-4-yl)-5-((3-(trifluoromethyl)pyridin-2-yl)methyl)pyrido[2,3-b]pyrazin-6(5H)-one: Followed general procedure F using tert-butyl 4-(8-methyl-6-oxo-5-((3-(trifluoromethyl)pyridin-2-yl)methyl)-5,6-dihydropyrido[2,3-b]pyrazin-7-yl)piperidine-1-carboxylate (80 mg, 0.159 mmol, 1 equiv) as the starting material to give the crude product 8-methyl-7-(piperidin-4-yl)-5-((3-(trifluoromethyl)pyridin-2-yl)methyl)pyrido[2,3-b]pyrazin-6(5H)-one (59.8 mg) as a yellow oil. MS m/z: 404 [M+H]⁺.

Step 3: 7-(1-(2-fluoro-6-methylphenyl)piperidin-4-yl)-8-methyl-5-((3-(trifluoromethyl)pyridin-2-yl)methyl)pyrido[2,3-b]pyrazin-6(5H)-one: Followed general procedure G using 3-methyl-7-(piperazin-1-yl)-5-((3-(trifluoromethyl)pyridin-2-yl)methyl)pyrido[2,3-b]pyrazin-6(5H)-one (59.8 mg, 0.148 mmol, 1 equiv) and 2-bromo-1-fluoro-3-methylbenzene (33.6 mg, 0.178 mmol, 1.2 equiv) as the starting materials to give 7-(1-(2-fluoro-6-methylphenyl)piperidin-4-yl)-8-methyl-5-((3-(trifluoromethyl)pyridin-2-yl)methyl)pyrido[2,3-b]pyrazin-6(5H)-one (30.1 mg, 39.0%) as a white solid. ¹H NMR (400 MHz, Chloroform-d) δ 8.46 (d, 1H), 8.41 (d, 1H), 8.30 (d, 1H), 7.96 (d, 1H), 7.24-7.21 (m, 1H), 7.04-6.93 (m, 2H), 6.92-6.82 (m, 1H), 6.02 (s, 2H), 3.32 (s, 3H), 3.15 (s, 2H), 2.79 (s, 5H), 2.41 (s, 3H), 2.03 (d, 1H), 1.66 (d, 2H). MS m/z: 512.15 [M+H]⁺.

### 7-(1-(2-Fluoro-6-methylphenyl)piperidin-4-yl)-3-methoxy-5-(2-(trifluoromethyl)benzyl)pyrido[2,3-b]pyrazin-6(5H)-one (58)

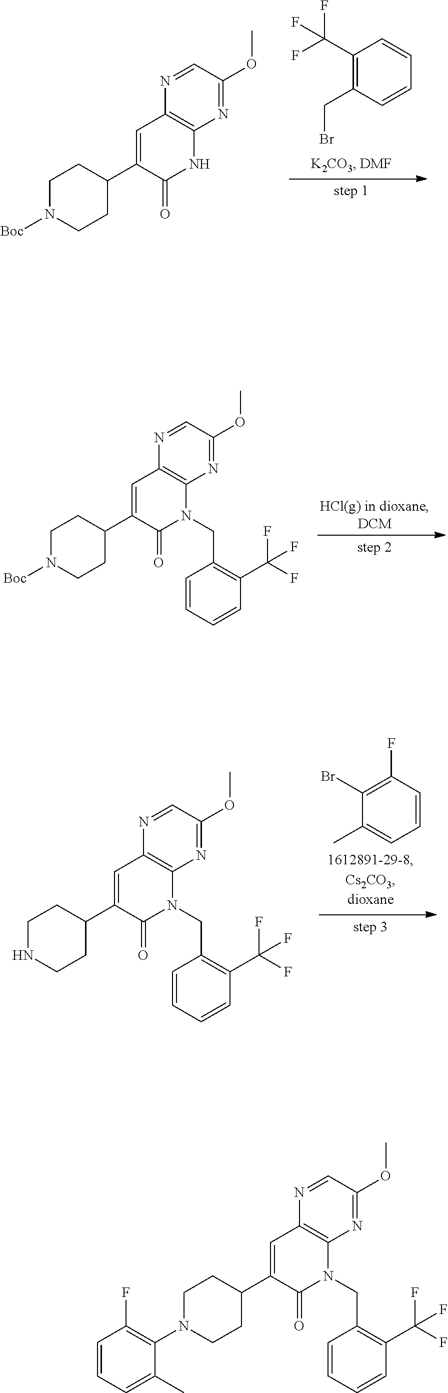

Step 1: tert-butyl 4-(3-methoxy-6-oxo-5-(2-(trifluoromethyl)benzyl)-5,6-dihydropyrido[2,3-b]pyrazin-7-yl)piperidine-1-carboxylate: Followed general procedure E using tert-butyl 4-(3-methoxy-6-oxo-5,6-dihydropyrido[2,3-b]pyrazin-7-yl)piperidine-1-carboxylate (100 mg, 0.277 mmol, 1 equiv) and 1-(bromomethyl)-2-(trifluoromethyl)benzene (99.4 mg, 0.416 mmol, 1.5 equiv) as the starting materials to give tert-butyl 4-(3-methoxy-6-oxo-5-(2-(trifluoromethyl)benzyl)-5,6-dihydropyrido[2,3-b]pyrazin-7-yl)piperidine-1-carboxylate (105 mg, 72%) as a yellow solid. MS m/z: 519 [M+H]⁺.

Step 2: 3-methoxy-7-(piperidin-4-yl)-5-(2-(trifluoromethyl)benzyl)pyrido[2,3-b]pyrazin-6(5H)-one: Followed general procedure F using tert-butyl 4-(3-methoxy-6-oxo-5-(2-(trifluoromethyl)benzyl)-5,6-dihydropyrido[2,3-b]pyrazin-7-yl)piperidine-1-carboxylate (100 mg, 0.193 mmol, 1 equiv) as the starting material to give the crude product 7-(1-(2-fluoro-6-methylphenyl)piperidin-4-yl)-3-methoxy-5-(2-(trifluoromethyl)benzyl)pyrido[2,3-b]pyrazin-6(5H)-one (80 mg) as a yellow oil. MS m/z: 419 [M+H]⁺.

Step 3: 7-(1-(2-fluoro-6-methylphenyl)piperidin-4-yl)-3-methoxy-5-(2-(trifluoromethyl)benzyl)pyrido[2,3-b]pyrazin-6(5H)-one: Followed general procedure G using 7-(1-(2-fluoro-6-methylphenyl)piperidin-4-yl)-3-methoxy-5-(2-(trifluoromethyl)benzyl)pyrido[2,3-b]pyrazin-6(5H)-one (80 mg, 0.191 mmol, 1 equiv) and 2-bromo-1-fluoro-3-methylbenzene (25.2 mg, 0.134 mmol, 1.5 equiv) as the starting materials to give 7-(1-(2-fluoro-6-methylphenyl)piperidin-4-yl)-3-methoxy-5-(2-(trifluoromethyl)benzyl)pyrido[2,3-b]pyrazin-6(5H)-one (28.5 mg, 26.0%) as an off-white solid. ¹H NMR (400 MHz, Chloroform-d) δ 8.10 (s, 1H), 7.88 (s, 1H), 7.74-7.68 (m, 1H), 7.37-7.25 (m, 2H), 7.02-6.93 (m, 2H), 6.91-6.81 (m, 1H), 6.76-6.68 (m, 1H), 5.90 (s, 2H), 3.76 (s, 3H), 3.40-3.29 (m, 2H), 3.22-3.06 (m, 3H), 2.36 (s, 3H), 2.12-2.03 (m, 2H), 1.87-1.75 (m, 2H). MS m/z: 527.15 [M+H]⁺.

### 7-(4-(5-Fluoro-2-methylpyridin-3-yl)piperazin-1-yl)-3-methyl-5-((3-(trifluoromethyl)pyrazin-2-yl)methyl)pyrido[2,3-b]pyrazin-6(5H)-one (59)

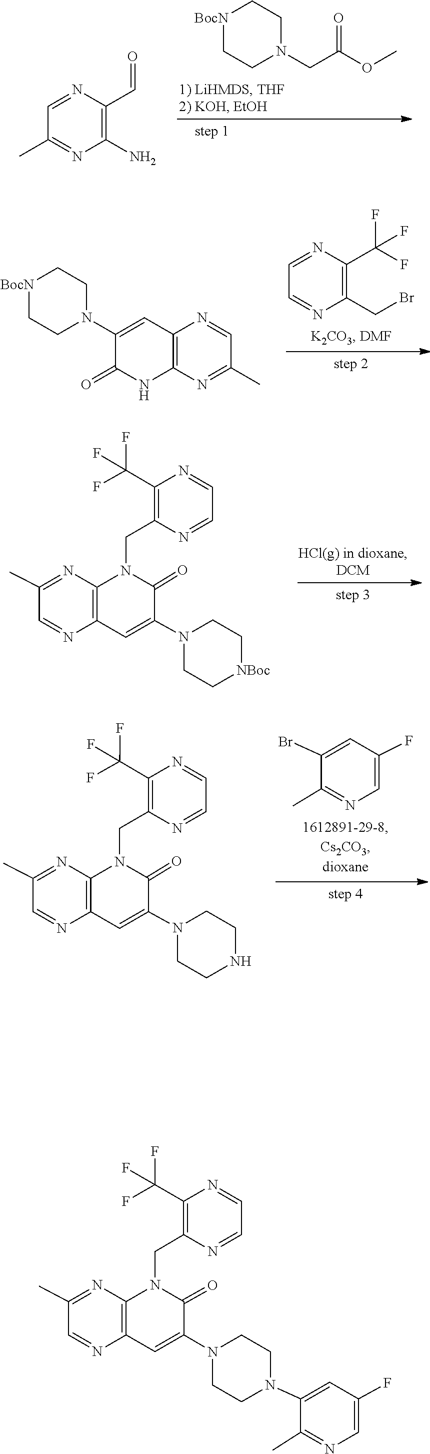

Step 1: tert-butyl 4-(3-methyl-6-oxo-5,6-dihydropyrido[2,3-b]pyrazin-7-yl)piperazine-1-carboxylate: Followed general procedure D using tert-butyl 4-(2-methoxy-2-oxoethyl)piperazine-1-carboxylate (452 mg, 1.75 mmol, 1.2 equiv) and 3-amino-5-methylpyrazine-2-carbaldehyde (200 mg, 1.45 mmol, 1 equiv) as the starting materials to give tert-butyl 4-(3-methyl-6-oxo-5,6-dihydropyrido[2,3-b]pyrazin-7-yl)piperazine-1-carboxylate (330 mg, 65%) as a light brown solid. MS m/z: 346 [M+H]⁺.

Step 2: tert-butyl 4-(3-methyl-6-oxo-5-((3-(trifluoromethyl)pyrazin-2-yl)methyl)-5,6-dihydropyrido[2,3-b]pyrazin-7-yl)piperazine-1-carboxylate: Followed general procedure E using tert-butyl 4-(3-methyl-6-oxo-5,6-dihydropyrido[2,3-b]pyrazin-7-yl)piperazine-1-carboxylate (90 mg, 0.261 mmol, 1 equiv) and 2-(bromomethyl)-3-(trifluoromethyl)pyrazine (94.2 mg, 0.392 mmol, 1.5 equiv) as the starting materials to give tert-butyl 4-(3-methyl-6-oxo-5-((3-(trifluoromethyl)pyrazin-2-yl)methyl)-5,6-dihydropyrido[2,3-b]pyrazin-7-yl)piperazine-1-carboxylate (70 mg, 53%) as an off-white solid. MS m/z: 506 [M+H]⁺.

Step 3: 3-methyl-7-(piperazin-1-yl)-5-((3-(trifluoromethyl)pyrazin-2-yl)methyl)pyrido[2,3-b]pyrazin-6(5H)-one: Followed general procedure F using tert-butyl 4-(3-methyl-6-oxo-5-((3-(trifluoromethyl)pyrazin-2-yl)methyl)-5,6-dihydropyrido[2,3-b]pyrazin-7-yl)piperazine-1-carboxylate (70 mg, 0.139 mmol, 1 equiv) as the starting material to give the crude product 3-methyl-7-(piperazin-1-yl)-5-((3-(trifluoromethyl)pyrazin-2-yl)methyl)pyrido[2,3-b]pyrazin-6(5H)-one (45 mg) as an off white solid. MS m/z: 406 [M+H]⁺.

Step 4: 7-(4-(5-fluoro-2-methylpyridin-3-yl)piperazin-1-yl)-3-methyl-5-((3-(trifluoromethyl)pyrazin-2-yl)methyl)pyrido[2,3-b]pyrazin-6(5H)-one: Followed general procedure G using 3-methyl-7-(piperazin-1-yl)-5-((3-(trifluoromethyl)pyrazin-2-yl)methyl)pyrido[2,3-b]pyrazin-6(5H)-one (45 mg, 0.11 mmol, 1 equiv) and 3-bromo-5-fluoro-2-methylpyridine (25.3 mg, 0.133 mmol, 1.2 equiv) as the starting materials to give 7-(4-(5-fluoro-2-methylpyridin-3-yl)piperazin-1-yl)-3-methyl-5-((3-(trifluoromethyl)pyrazin-2-yl)methyl)pyrido[2,3-b]pyrazin-6(5H)-one (19.4 mg, 32.9%) as a orange solid. ¹H NMR (400 MHz, Chloroform-d) δ 8.55-8.49 (m, 1H), 8.48-8.44 (m, 1H), 8.31 (s, 1H), 8.12 (d, 1H), 7.25 (s, 1H), 7.10 (dd, 1H), 6.10 (s, 2H), 3.60-3.45 (m, 4H), 3.20-3.08 (m, 4H), 2.61-2.54 (m, 3H), 2.49 (s, 3H). MS m/z: 515.15 [M+H]⁺.

### 5-((3-(Trifluoromethyl)pyridin-2-yl)methyl)-7-(1-(4-(trifluoromethyl)pyridin-3-yl)piperi-din-4-yl)pyrido[2,3-b]pyrazin-6(5H)-one (60)

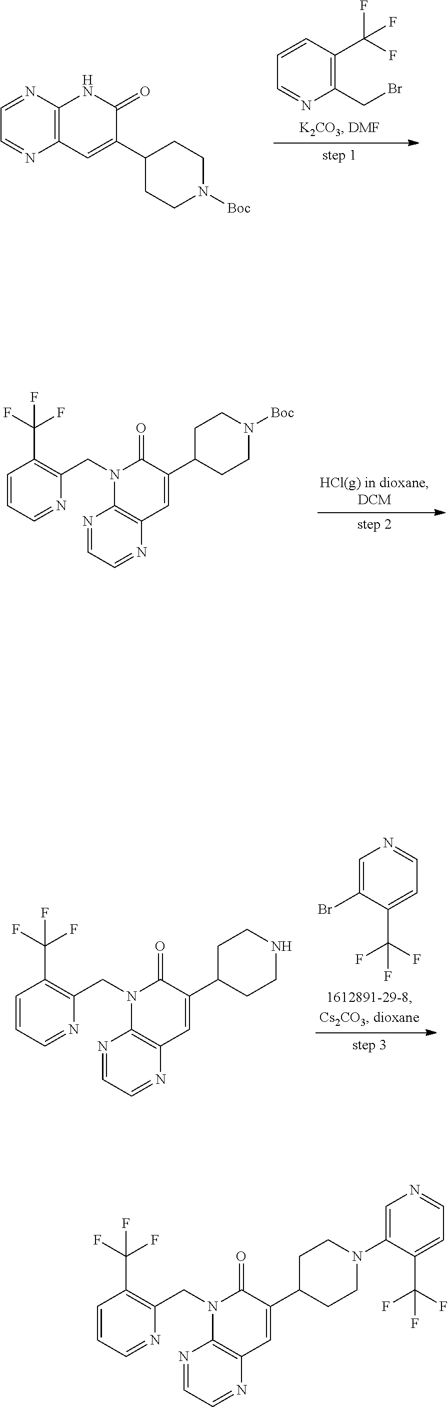 [SMILES: CC(C)(C)OC(=O)N1CCC(c2cc3nccnc3[nH]c2=O)CC1 ; FC(F)(F)c1cccnc1CBr ; CC(C)(C)OC(=O)N1CCC(c2cc3nccnc3n(Cc3ncccc3C(F)(F)F)c2=O)CC1 ; O=c1c(C2CCNCC2)cc2nccnc2n1Cc1ncccc1C(F)(F)F ; FC(F)(F)c1ccncc1Br ; O=c1c(C2CCN(c3cnccc3C(F)(F)F)CC2)cc2nccnc2n1Cc1ncccc1C(F)(F)F]

Step 1: tert-butyl 4-(6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (200 mg, 0.605 mmol, 1 equiv) and 2-(bromomethyl)-3-(trifluoromethyl)pyridine (145 mg, 0.605 mmol, 1 equiv) as the starting materials to give tert-butyl 4-(6-oxo-5-((3-(trifluoromethyl)pyridin-2-yl)methyl)-5,6-dihydropyrido[2,3-b]pyrazin-7-yl)piperidine-1-carboxylate (170 mg, 57%) as a yellow solid. MS m/z: 490 [M+H]⁺.

Step 2: 7-(piperidin-4-yl)-5-((3-(trifluoromethyl)pyridin-2-yl)methyl)pyrido[2,3-b]pyrazin-6(5H)-one: Followed general procedure F using tert-butyl 4-(6-oxo-5-((3-(trifluoromethyl)pyridin-2-yl)methyl)-5,6-dihydropyrido[2,3-b]pyrazin-7-yl)piperidine-1-carboxylate (170 mg, 0.347 mmol, 1 equiv) as the starting material to give the crude product 7-(piperidin-4-yl)-5-((3-(trifluoromethyl)pyridin-2-yl)methyl)pyrido[2,3-b]pyrazin-6(5H)-one (120 mg) as an off-white oil. MS m/z: 390 [M+H]⁺.

Step 3: 5-((3-(trifluoromethyl)pyridin-2-yl)methyl)-7-(1-(4-(trifluoromethyl)pyridin-3-yl)piperidin-4-yl)pyrido[2,3-b]pyrazin-6(5H)-one: Followed general procedure G using 7-(piperidin-4-yl)-5-((3-(trifluoromethyl)pyridin-2-yl)methyl)-pyrido[2,3-b]pyrazin-6(5H)-one (60 mg, 0.141 mmol, 1 equiv) and 3-bromo-4-(trifluoromethyl)pyridine (35 mg, 0.155 mmol, 1.1 equiv) as the starting materials to give 5-((3-(trifluoromethyl)pyridin-2-yl)methyl)-7-(1-(4-(trifluoromethyl)pyridin-3-yl)piperidin-4-yl)pyrido[2,3-b]pyrazin-6(5H)-one (28.1 mg, 36.9%) as an off-white solid. ¹H NMR (300 MHz, Chloroform-d) δ 8.69 (s, 1H), 8.55-8.48 (m, 2H), 8.45-8.41 (m, 1H), 8.41-8.37 (m, 1H), 8.03-7.97 (m, 1H), 7.92 (d, 1H), 7.52 (d, 1H), 7.28-7.22 (m, 1H), 6.07 (s, 2H), 3.40-3.30 (m, 2H), 3.27-3.16 (m, 1H), 3.16-3.05 (m, 2H), 2.19-2.08 (m, 2H), 1.89 (qd, 2H). MS m/z: 535.15 [M+H]⁺.

### (S)-7-(1-(2-Fluoro-6-methylphenyl)piperidin-4-yl)-3-methyl-5-(5,6,7,8-tetrahydroquinoxalin-5-yl) pyrido[2,3-b]pyrazin-6(5H)-one (61a); (R)-7-(1-(2-fluoro-6-methylphenyl) piperidin-4-yl)-3-methyl-5-(5,6,7,8-tetrahydroquinoxalin-5-yl)pyrido[2,3-b]pyrazin-6(5H)-one (61b)

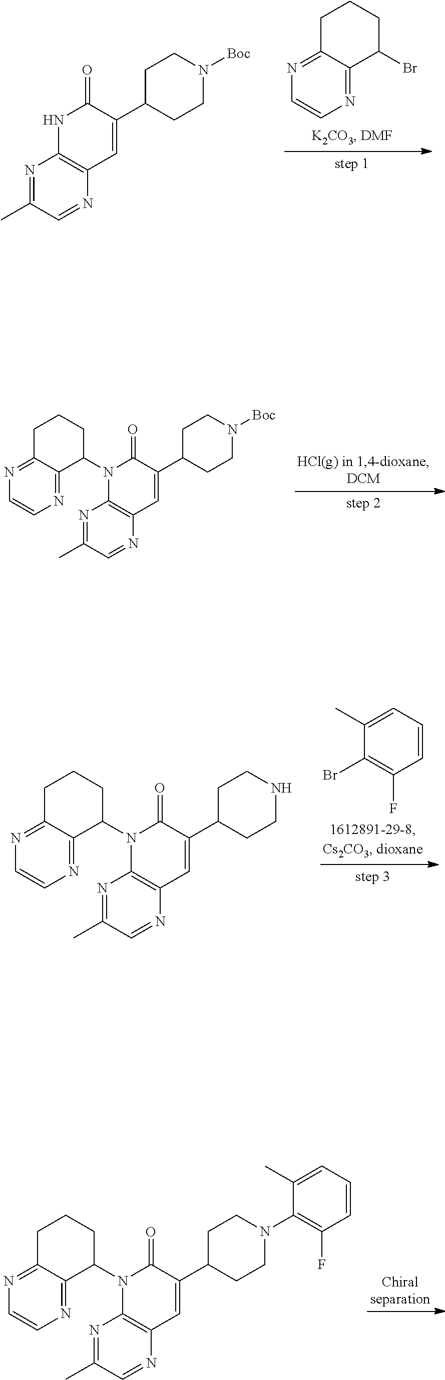

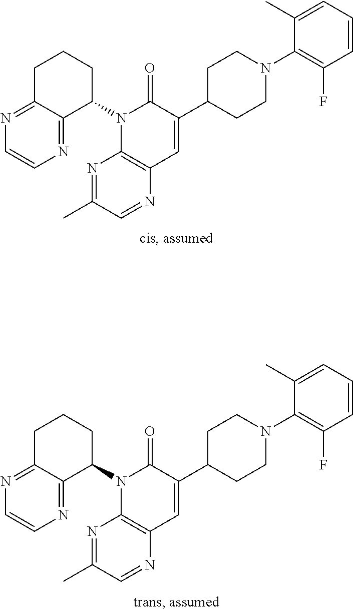 [SMILES: Cc1cnc2cc(C3CCN(c4c(C)cccc4F)CC3)c(=O)n([C@H]3CCCc4nccnc43)c2n1 ; Cc1cnc2cc(C3CCN(c4c(C)cccc4F)CC3)c(=O)n([C@@H]3CCCc4nccnc43)c2n1]

Step 1: tert-butyl 4-(3-methyl-6-oxo-5-(5,6,7,8-tetrahydroquinoxalin-5-yl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (260 mg, 0.755 mmol, 1 equiv) and 5-bromo-5,6,7,8-tetrahydroquinoxaline (241 mg, 1.13 mmol, 1.5 equiv) as the starting materials to give tert-butyl 4-(3-methyl-6-oxo-5-(5,6,7,8-tetrahydroquinoxalin-5-yl)-5,6-dihydropyrido[2,3-b]pyrazine-7-yl) piperidine-1-carboxylate (160 mg, 44%) as a yellow oil. MS m/z: 477[M+H]⁺.

Step 2: 3-methyl-7-(piperidin-4-yl)-5-(5,6,7,8-tetrahydroquinoxalin-5-yl)pyrido[2,3-b]pyrazin-6(5H)-one: Followed general procedure F using tert-butyl 4-(3-methyl-6-oxo-5-(5,6,7,8-tetrahydroquinoxalin-5-yl)-5,6-dihydropyrido[2,3-b]pyrazine-7-yl)piperidine-1-carboxylate (160 mg, 0.336 mmol, 1 equiv) as the starting material to give the crude product 3-methyl-7-(piperidin-4-yl)-5-(5,6,7,8-tetrahydroquinoxalin-5-yl)pyrido[2,3-b]pyrazin-6(5H)-one (110 mg) as a yellow oil. MS m/z: 377 [M+H]⁺.

Step 3: 7-(1-(2-fluoro-6-methylphenyl)piperidin-4-yl)-3-methyl-5-(5,6,7,8-tetrahydroquinoxalin-5-yl)pyrido[2,3-b]pyrazin-6(5H)-one: Followed general procedure G using 3-methyl-7-(piperidin-4-yl)-5-(5,6,7,8-tetrahydroquinoxalin-5-yl)pyrido[2,3-b]pyrazin-6(5H)-one (110 mg, 0.292 mmol, 1 equiv) and 2-bromo-1-fluoro-3-methyl-benzene (66.2 mg, 0.35 mmol, 1.2 equiv) as the starting materials to give the crude product 7-(1-(2-fluoro-6-methylphenyl)piperidin-4-yl)-3-methyl-5-(5,6,7,8-tetrahydroquinoxalin-5-yl)pyrido[2,3-b]pyrazin-6(5H)-one (100 mg) as a yellow solid. The crude product was purified by Prep-HPLC with the following conditions (Column: CHIRAL ART Cellulose-SC, 2*25 cm, 5 m; Mobile Phase A: Hex (10 mM NH₃-MeOH), Mobile Phase B: IPA-HPLC; Flow rate: 20 mL/min; Gradient: 30% B to 30% B in 15 min; Wave Length: 212/205 nm; RT1 (min): 7.875; RT2 (min): 10.585; Sample Solvent: IPA-HPLC; Injection Volume: 0.6 mL; Number Of Runs: 5) to afford (S)-7-(1-(2-fluoro-6-methylphenyl)piperidin-4-yl)-3-methyl-5-(5,6,7,8-tetrahydroquinoxalin-5-yl)pyrido[2,3-b]pyrazin-6(5H)-one (61a, 31.2 mg, 21.9%) as a light yellow solid and (R)-7-(1-(2-fluoro-6-methylphenyl)piperidin-4-yl)-3-methyl-5-(5,6,7,8-tetrahy-droquinoxalin-5-yl)pyrido[2,3-b]pyrazin-6(5H)-one (61b, 33.3 mg, 23.4%) as a light yellow solid.

(S)-7-(1-(2-fluoro-6-methylphenyl)piperidin-4-yl)-3-methyl-5-(5,6,7,8-tetrahydroquinoxalin-5-yl)pyrido[2,3-b]pyrazin-6(5H)-one (61a)¹H NMR (400 MHz, Chloroform-d) δ 8.40-8.07 (m, 3H), 7.80 (d, 1H), 7.05-6.72 (m, 4H), 3.43-2.94 (m, 7H), 2.67-2.52 (m, 3H), 2.38 (d, 3H), 2.30-2.21 (m, 2H), 2.18 (s, 1H), 2.13-2.02 (m, 2H), 1.97 (d, 1H), 1.85-1.75 (m, 2H). MS m/z: 485.2 [M+H]+

(R)-7-(1-(2-fluoro-6-methylphenyl)piperidin-4-yl)-3-methyl-5-(5,6,7,8-tetrahydroquinoxalin-5-yl)pyrido[2,3-b]pyrazin-6(5H)-one (61b)¹H NMR (400 MHz, Chloroform-d) δ 8.41-8.08 (m, 3H), 7.81 (d, 1H), 7.05-6.72 (m, 4H), 3.45-2.94 (m, 7H), 2.67-2.63 (m, 3H), 2.39 (d, 3H), 2.31-2.22 (m, 2H), 2.19 (s, 1H), 2.14-1.95 (m, 3H), 1.85-1.79 (s, 2H). MS m/z: 485.2 [M+H]⁺

### 7-(1-(2-Fluoro-4-methylpyridin-3-yl)piperidin-4-yl)-8-methyl-5-((3-(trifluoromethyl)pyridin-2-yl)methyl)pyrido[2,3-b]pyrazin-6(5H)-one (62)

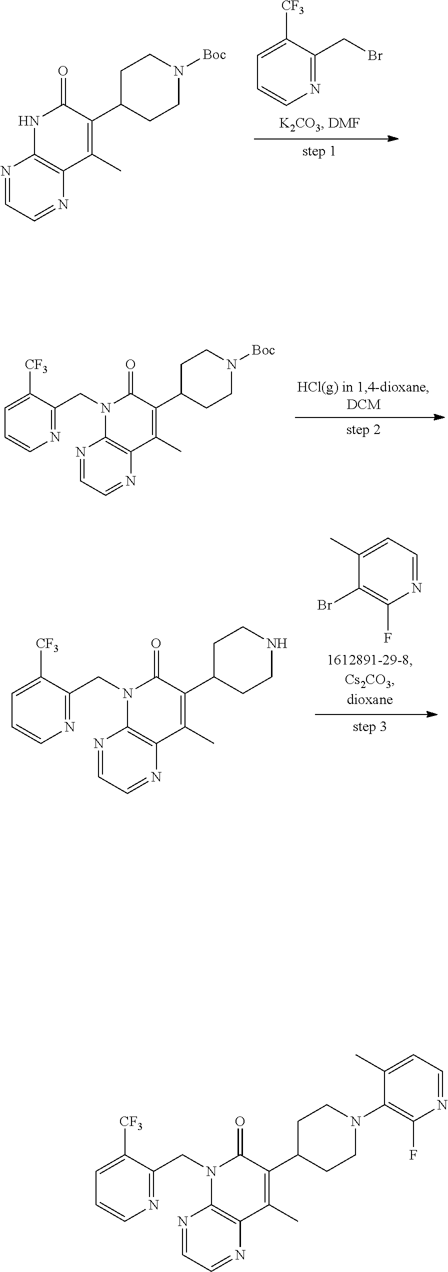

Step 1: tert-butyl 4-(8-methyl-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (100 mg, 0.29 mmol, 1 equiv) and 2-(bromomethyl)-3-(trifluoromethyl)pyridine hydrobromide (83.9 mg, 0.261 mmol, 0.9 equiv) as the starting materials to give tert-butyl 4-(8-methyl-6-oxo-5-((3-(trifluoromethyl)pyridin-2-yl)methyl)-5,6-dihydropyrido[2,3-b]pyrazin-7-yl)piperidine-1-carboxylate (80 mg, 54%) as a yellow oil. MS m/z: 504 [M+H]⁺.

Step 2: 8-methyl-7-(piperidin-4-yl)-5-((3-(trifluoromethyl)pyridin-2-yl)methyl)pyrido[2,3-b]pyrazin-6(5H)-one: Followed general procedure F using tert-butyl 4-(8-methyl-6-oxo-5-((3-(trifluoromethyl)pyridin-2-yl)methyl)-5,6-dihydropyrido[2,3-b]pyrazin-7-yl)piperidine-1-carboxylate (80 mg, 0.159 mmol, 1 equiv) as the starting material to give the crude product 8-methyl-7-(piperidin-4-yl)-5-((3-(trifluoromethyl)pyridin-2-yl)methyl)pyrido[2,3-b]pyrazin-6(5H)-one (59.8 mg) as a yellow oil. MS m/z: 404 [M+H]⁺.

Step 3: 7-(1-(2-fluoro-4-methylpyridin-3-yl)piperidin-4-yl)-8-methyl-5-((3-(trifluoromethyl)-pyridin-2-yl)methyl)pyrido[2,3-b]pyrazin-6(5H)-one: Followed general procedure G using 3-methyl-7-(piperazin-1-yl)-5-((3-(trifluoromethyl)pyridin-2-yl)methyl)pyrido[2,3-b]pyrazin-6(5H)-one (59.8 mg, 0.148 mmol, 1 equiv) and 3-bromo-2-fluoro-4-methylpyridine (31.1 mg, 0.164 mmol, 1.1 equiv) as the starting materials to give 7-(1-(2-fluoro-4-methylpyridin-3-yl)piperidin-4-yl)-8-methyl-5-((3-(trifluoromethyl)pyridin-2-yl)methyl)pyrido[2,3-b]pyrazin-6(5H)-one (30.1 mg, 39%) as a white solid. ¹H NMR (400 MHz, Chloroform-d) δ 8.46 (d, 1H), 8.44-8.38 (m, 1H), 8.30 (d, 1H), 8.00-7.93 (m, 1H), 7.83-7.77 (m, 1H), 7.25-7.22 (m, 1H), 6.99 (d, 1H), 6.02 (s, 2H), 3.31-3.17 (m, 3H), 3.02 (d, 2H), 2.76 (s, 5H), 2.40 (s, 3H), 1.65 (d, 2H). MS m/z: 513.2 [M+H]⁺.

### 7-(1-(2-Fluoro-4-methylpyridin-3-yl)piperidin-4-yl)-3,8-dimethyl-5-((3-(trifluoromethyl) pyrazin-2-yl)methyl)pyrido[2,3-b]pyrazin-6(5H)-one (63)

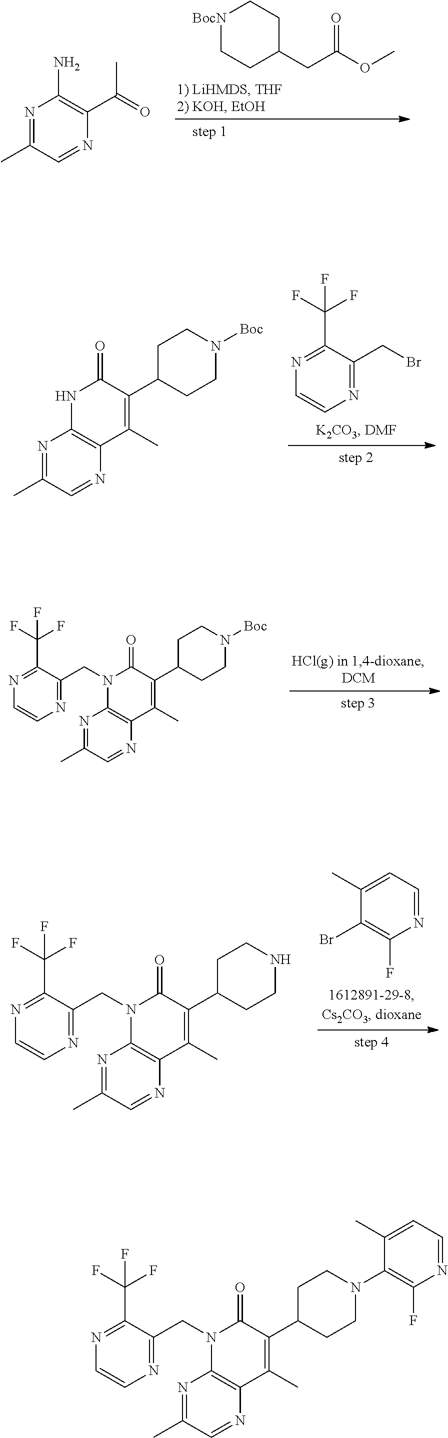

Step 1: tert-butyl 4-(3,8-dimethyl-6-oxo-5,6-dihydropyrido[2,3-b]pyrazin-7-yl)piperidine-1-carboxylate: Followed general procedure D using tert-butyl 4-(2-methoxy-2-oxoethyl)piperidine-1-carboxylate (536 mg, 2.08 mmol, 1.5 equiv) and 1-(3-amino-5-methylpyrazin-2-yl)ethan-1-one (210 mg, 1.38 mmol, 1 equiv) as the starting materials to give tert-butyl 4-(3,8-dimethyl-6-oxo-5,6-dihydropyrido[2,3-b]pyrazin-7-yl)piperidine-1-carboxylate (258 mg, 51%) as a yellow solid. MS m/z: 359 [M+H]⁺.

Step 2: tert-butyl 4-(3,8-dimethyl-6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(3,8-dimethyl-6-oxo-5,6-dihydropyrido[2,3-b]pyrazin-7-yl)piperidine-1-carboxylate (80 mg, 0.223 mmol, 1 equiv) and 2-(bromomethyl)-3-(trifluoromethyl)pyrazine (64.5 mg, 0.268 mmol, 1.2 equiv) as the starting material to give tert-butyl 4-(3,8-dimethyl-6-oxo-5-((3-(trifluoromethyl)pyrazin-2-yl)methyl)-5,6-dihydropyrido[2,3-b]pyrazin-7-yl)piperidine-1-carboxylate (76 mg, 65%) as a yellow oil. MS m/z: 519 [M+H]⁺.

Step 3: 3,8-dimethyl-7-(piperidin-4-yl)-5-((3-(trifluoromethyl)pyrazin-2-yl)methyl)pyrido[2,3-b]pyrazin-6(5H)-one: Followed general procedure F using tert-butyl 4-(3,8-dimethyl-6-oxo-5-((3-(trifluoromethyl)pyrazin-2-yl)methyl)-5,6-dihydropyrido[2,3-b]pyrazin-7-yl)piperidine-1-carboxylate (76 mg, 0.147 mmol, 1 equiv) as the starting material to give the crude product 3,8-dimethyl-7-(piperidin-4-yl)-5-((3-(trifluoromethyl)pyrazin-2-yl)methyl)pyrido[2,3-b]pyrazin-6(5H)-one (50 mg) as a yellow oil. MS m/z: 419 [M+H]⁺.

Step 4: 7-(1-(2-fluoro-4-methylpyridin-3-yl)piperidin-4-yl)-3,8-dimethyl-5-((3-(trifluoromethyl) pyrazin-2-yl)methyl)pyrido[2,3-b]pyrazin-6(5H)-one: Followed general procedure G using 3,8-dimethyl-7-(piperidin-4-yl)-5-((3-(trifluoromethyl)pyrazin-2-yl)methyl)pyrido[2,3-b]pyrazin-6(5H)-one (50 mg, 0.119 mmol, 1 equiv) and 3-bromo-2-fluoro-4-methylpyridine (27.2 mg, 0.143 mmol, 1.2 equiv) as the starting materials to give 7-(1-(2-fluoro-4-methylpyridin-3-yl)piperidin-4-yl)-3,8-dimethyl-5-((3-(trifluoromethyl)pyrazin-2-yl)methyl)pyrido[2,3-b]pyrazin-6(5H)-one (15.5 mg, 24.4%) as a pink solid. ¹H NMR (400 MHz, Chloroform-d) δ 8.50-8.45 (m, 2H), 8.34 (s, 1H), 7.82-7.80 (m, 1H), 7.00 (d, 1H), 6.06 (s, 2H), 3.30-3.21 (m, 3H), 3.04 (d, 2H), 2.78-2.68 (m, 5H), 2.47 (s, 3H), 2.42 (d, 2H), 1.66 (s, 3H). MS m/z: 528.15 [M+H]⁺.

### 7-(1-(2-(Trifluoromethyl)phenyl)piperidin-4-yl)-5-((3-(trifluoromethyl)pyrazin-2-yl)methyl)pyrido[2,3-b]pyrazin-6(5H)-one (64)

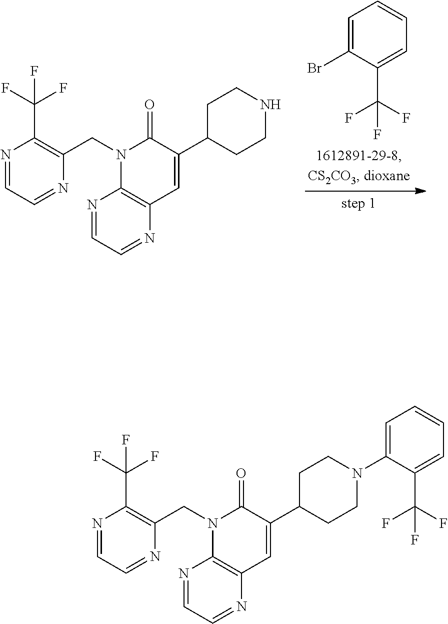 [SMILES: O=c1c(C2CCNCC2)cc2nccnc2n1Cc1nccnc1C(F)(F)F ; FC(F)(F)c1ccccc1Br ; O=c1c(C2CCN(c3ccccc3C(F)(F)F)CC2)cc2nccnc2n1Cc1nccnc1C(F)(F)F]

Step 1: 7-(1-(2-(trifluoromethyl)phenyl)piperidin-4-yl)-5-((3-(trifluoromethyl)pyrazin-2-yl)methyl)pyrido[2,3-b]pyrazin-6(5H)-one: Followed general procedure G using 7-(piperidin-4-yl)-5-((3-(trifluoromethyl)pyrazin-2-yl)methyl)pyrido[2,3-b]pyrazin-6(5H)-one (60 mg, 0.154 mmol, 1 equiv) and 1-bromo-2-(trifluoromethyl)benzene (41.6 mg, 0.185 mmol, 1.2 equiv) as the starting materials to give 7-(1-(2-(trifluoromethyl)phenyl)piperidin-4-yl)-5-((3-(trifluoromethyl)pyrazin-2-yl)methyl)pyrido[2,3-b]pyrazin-6(5H)-one (47.8 mg, 57.5%) as an off-white solid. ¹H NMR (400 MHz, Chloroform-d) δ 8.51 (dd, 2H), 8.44 (d, 1H), 8.35 (d, 1H), 7.93 (d, 1H), 7.67-7.60 (m, 1H), 7.57-7.49 (m, 1H), 7.40 (d, 1H), 7.25-7.18 (m, 1H), 6.09 (s, 2H), 3.26-3.08 (m, 3H), 2.98-2.89 (m, 2H), 2.11-2.02 (m, 2H), 1.95-1.83 (m, 2H). MS m/z: 535.15 [M+H]⁺.

### 7-(1-(2-Fluoro-6-methylphenyl)piperidin-4-yl)-2-methoxy-5-((3-(trifluoromethyl)pyridin-2-yl)methyl)pyrido[2,3-b]pyrazin-6(5H)-one (65)

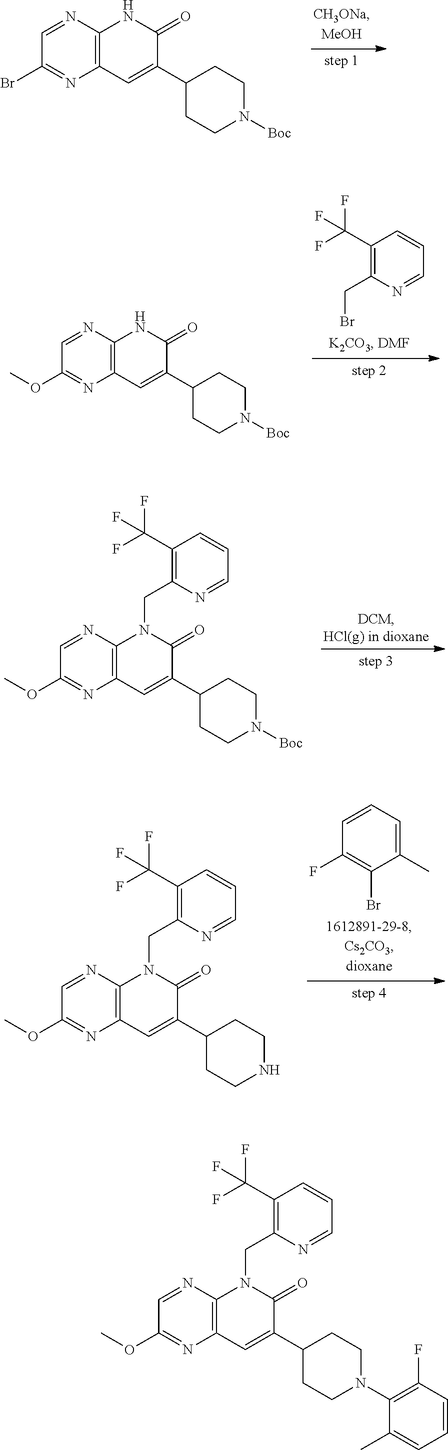

Step 1: tert-butyl 4-(2-methoxy-6-oxo-5,6-dihydropyrido[2,3-b]pyrazin-7-yl)piperidine-1-carboxylate: Followed general procedure D using tert-butyl 4-(2-bromo-6-oxo-5,6-dihydropyrido[2,3-b]pyrazin-7-yl)piperidine-1-carboxylate (100 mg, 0.244 mmol, 1 equiv) and sodium methoxide (39.6 mg, 0.732 mmol, 3 equiv) as the starting materials to give tert-butyl 4-(2-methoxy-6-oxo-5,6-dihydropyrido[2,3-b]pyrazin-7-yl)piperidine-1-carboxylate (60 mg, 68%) as a yellow solid. MS m/z: 361 [M+H]⁺.

Step 2: tert-butyl 4-(2-methoxy-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(2-methoxy-6-oxo-5,6-dihydropyrido[2,3-b]pyrazin-7-yl)piperidine-1-carboxylate (60 mg, 0.166 mmol, 1 equiv) and 2-(bromomethyl)-3-(trifluoromethyl)pyridine (39.9 mg, 0.166 mmol, 1 equiv) as the starting materials to give tert-butyl 4-(2-methoxy-6-oxo-5-((3-(trifluoromethyl)pyridin-2-yl)methyl)-5,6-dihydropyrido[2,3-b]pyrazin-7-yl)piperidine-1-carboxylate (60 mg, 69%) as an off-white solid. MS m/z: 520 [M+H]⁺.

Step 3: 2-methoxy-7-(piperidin-4-yl)-5-((3-(trifluoromethyl)pyridin-2-yl)methyl)pyrido[2,3-b]pyrazin-6(5H)-one: Followed general procedure F using tert-butyl 4-(2-methoxy-6-oxo-5-((3-(trifluoromethyl)pyridin-2-yl)methyl)-5,6-dihydropyrido[2,3-b]pyrazin-7-yl)piperidine-1-carboxylate (60 mg, 0.115 mmol, 1 equiv) as the starting material to give the crude product 2-methoxy-7-(piperidin-4-yl)-5-((3-(trifluoromethyl) pyridin-2-yl)methyl)pyrido[2,3-b]pyrazin-6(5H)-one (40 mg) as a yellow oil. MS m/z: 420 [M+H]⁺.

Step 4: 7-(1-(2-fluoro-6-methylphenyl)piperidin-4-yl)-2-methoxy-5-((3-(trifluoromethyl) pyridin-2-yl)methyl)pyrido[2,3-b]pyrazin-6(5H)-one: Followed general procedure G using 2-methoxy-7-(piperidin-4-yl)-5-((3-(trifluoromethyl)pyridin-2-yl)methyl)pyrido[2,3-b]pyrazin-6(5H)-one (40 mg, 0.088 mmol, 1 equiv) and 2-bromo-1-fluoro-3-methylbenzene (18.2 mg, 0.097 mmol, 1.1 equiv) as the starting materials to give 7-(1-(2-fluoro-6-methylphenyl)piperidin-4-yl)-2-methoxy-5-((3-(trifluoromethyl)pyridin-2-yl)methyl)pyrido[2,3-b]pyrazin-6(5H)-one (15.3 mg, 32.1%) as a yellow solid. ¹H NMR (300 MHz, Chloroform-d) δ 8.50-8.35 (m, 1H), 8.13-8.02 (m, 1H), 7.99 (d, 1H), 7.82 (s, 1H), 7.27-7.17 (m, 1H), 7.03-6.94 (m, 2H), 6.93-6.80 (m, 1H), 6.06 (s, 2H), 4.06 (s, 3H), 3.45-3.27 (m, 2H), 3.27-2.95 (m, 3H), 2.40 (s, 3H), 2.16-1.98 (m, 2H), 1.97-1.75 (m, 2H). MS m/z: 528.15 [M+H]⁺.

### 5-((3-(Difluoromethoxy)pyridin-2-yl)methyl)-7-(1-(2-fluoro-6-methylphenyl)piperidin-4-yl)-3-methylpyrido[2,3-b]pyrazin-6(5H)-one (66)

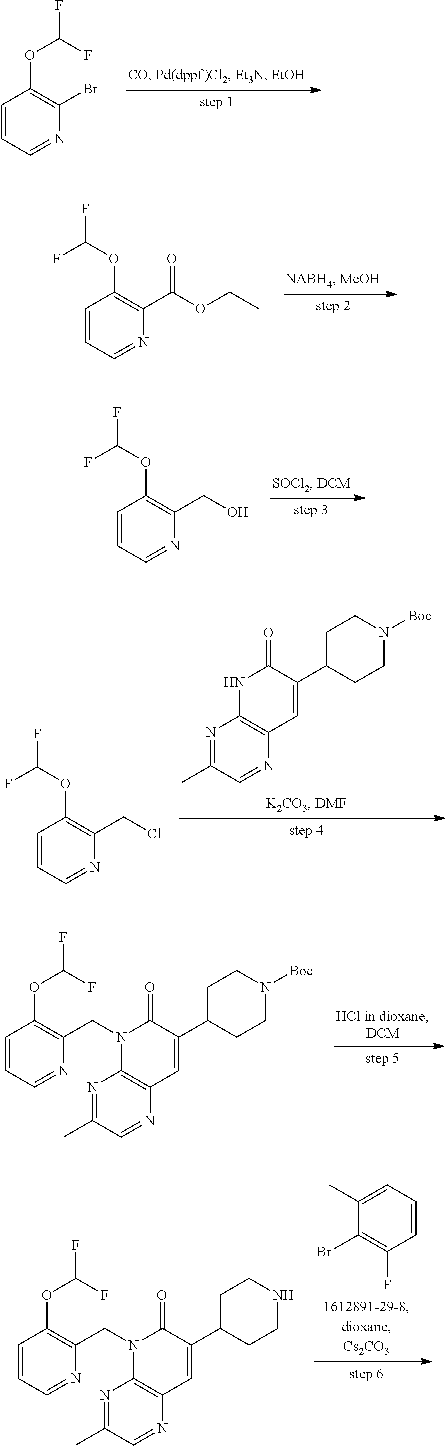

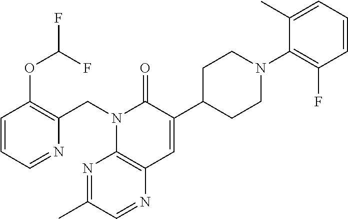 [SMILES: Cc1cnc2cc(C3CCN(c4c(C)cccc4F)CC3)c(=O)n(Cc3ncccc3OC(F)F)c2n1]

Step 1: ethyl 3-(difluoromethoxy)picolinate: Followed general procedure H using 2-bromo-3-(difluoromethoxy)pyridine (200 mg, 0.893 mmol, 1 equiv) as the starting material to give ethyl 3-(difluoromethoxy)picolinate (180 mg, 92%) as a light yellow oil. MS m/z: 218 [M+H]⁺.

Step 2: (3-(difluoromethoxy)pyridin-2-yl)methanol: Followed general procedure I using 3-(difluoromethoxy)picolinate (180 mg, 0.829 mmol, 1 equiv) as the starting material to give (3-(difluoromethoxy)pyridin-2-yl)methanol (100 mg, 68%) as a colorless oil. MS m/z: 176 [M+H]⁺.

Step 3: 2-(chloromethyl)-3-(difluoromethoxy)pyridine: Followed general procedure J using (3-(difluoromethoxy)pyridin-2-yl)methanol (100 mg, 0.571 mmol, 1 equiv) as the starting material to give the crude product 2-(chloromethyl)-3-(difluoromethoxy)pyridine (100 mg) as a white solid. MS m/z: 194 [M+H]⁺.

Step 4: tert-butyl 4-(5-((3-(difluoromethoxy)pyridin-2-yl)methyl)-3-methyl-6-oxo-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (90 mg, 0.261 mmol, 1 equiv) and 2-(chloromethyl)-3-(difluoromethoxy)pyridine (75.5 mg, 0.391 mmol, 1.5 equiv) as the starting materials to give tert-butyl 4-(5-((3-(difluoromethoxy)pyridin-2-yl)methyl)-3-methyl-6-oxo-5,6-dihydropyrido[2,3-b]pyrazin-7-yl)piperidine-1-carboxylate (70 mg, 53%) as an off-white solid. MS m/z: 502 [M+H]⁺.

Step 5: 5-((3-(difluoromethoxy)pyridin-2-yl)methyl)-3-methyl-7-(piperidin-4-yl)pyrido[2,3-b]pyrazin-6(5H)-one: Followed general procedure F using tert-butyl 4-(5-((3-(difluoromethoxy)pyridin-2-yl)methyl)-3-methyl-6-oxo-5,6-dihydropyrido[2,3-b]pyrazin-7-yl)piperidine-1-carboxylate (70 mg, 0.139 mmol, 1 equiv) as the starting material to give the crude product 5-((3-(difluoromethoxy)pyridin-2-yl)methyl)-3-methyl-7-(piperidin-4-yl)pyrido[2,3-b]pyrazin-6(5H)-one (45 mg) as an off white solid. MS m/z: 402 [M+H]⁺.

Step 6: 5-((3-(difluoromethoxy)pyridin-2-yl)methyl)-7-(1-(2-fluoro-6-methylphenyl)piperidin-4-yl)-3-methylpyrido[2,3-b]pyrazin-6(5H)-one: Followed general procedure G using 5-((3-(difluoromethoxy)pyridin-2-yl)methyl)-3-methyl-7-(piperidin-4-yl)pyrido[2,3-b]pyrazin-6(5H)-one (45 mg, 0.112 mmol, 1 equiv) and 3-bromo-5-fluoro-2-methylpyridine (31.7 mg, 0.168 mmol, 1.5 equiv) as the starting materials to give 5-((3-(difluoromethoxy)pyridin-2-yl)methyl)-7-(1-(2-fluoro-6-methylphenyl)piperidin-4-yl)-3-methylpyrido[2,3-b]pyrazin-6(5H)-one (24.5 mg, 39.7%) as an orange solid. ¹H NMR (400 MHz, Chloroform-d) δ 8.34 (s, 1H), 8.20 (dd, 1H), 7.84 (d, 1H), 7.53-7.47 (m, 1H), 7.19-7.13 (m, 1H), 6.99-6.93 (m, 2H), 6.91-6.55 (m, 2H), 5.92 (s, 2H), 3.37-3.26 (m, 2H), 3.19-3.05 (m, 3H), 2.55 (s, 3H), 2.36 (s, 3H), 2.04-1.98 (m, 2H), 1.85-1.74 (m, 2H). MS m/z: 510.20 [M+H]⁺.

### 7-(4-(2-Fluoro-6-methylphenyl)piperazin-1-yl)-3-methyl-5-((3-(trifluoromethyl)pyridin-2-yl)methyl)pyrido[2,3-b]pyrazin-6(5H)-one (67)

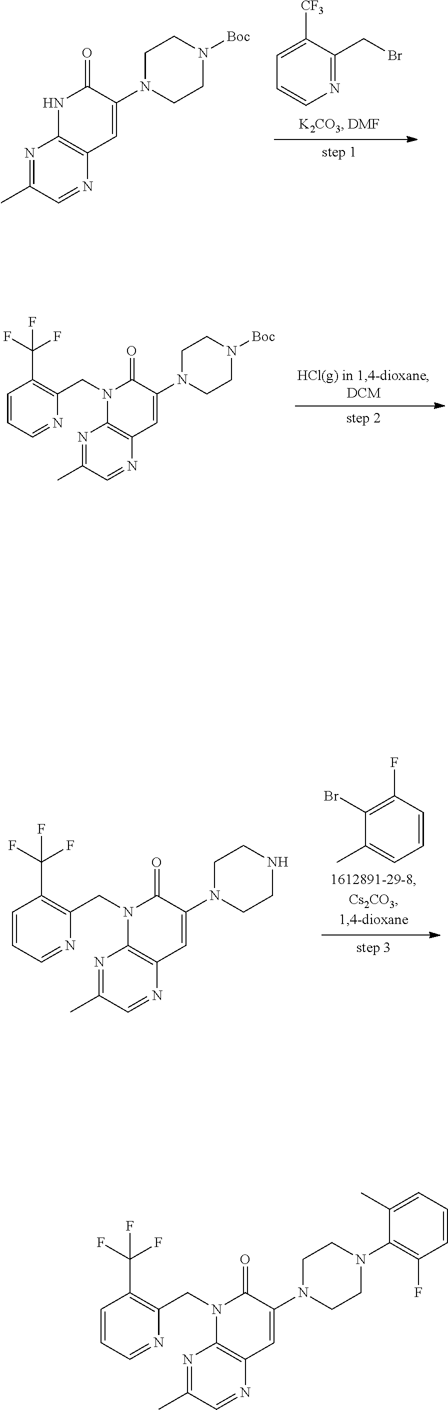

Step 1: tert-butyl 4-(3-methyl-6-oxo-5-((3-(trifluoromethyl)pyridin-2-yl)methyl)-5,6-dihydropyrido[2,3-b]pyrazin-7-yl)piperazine-1-carboxylate: Followed general procedure E using tert-butyl 4-(3-methyl-6-oxo-5,6-dihydropyrido[2,3-b]pyrazin-7-yl)piperazine-1-carboxylate (100 mg, 0.29 mmol, 1 equiv) and 2-(bromomethyl)-3-(trifluoromethyl)pyridine hydrogen bromide (92.3 mg, 0.29 mmol, 1 equiv) as the starting materials to give tert-butyl 4-(3-methyl-6-oxo-5-((3-(trifluoromethyl)pyridin-2-yl)methyl)-5,6-dihydropyrido[2,3-b]pyrazin-7-yl)piperazine-1-carboxylate (50 mg, 34%) as a yellow solid. MS m/z: 505 [M+H]⁺.

Step 2: 3-methyl-7-(piperazin-1-yl)-5-((3-(trifluoromethyl)pyridin-2-yl)methyl)pyrido[2,3-b]pyrazin-6(5H)-one: Followed general procedure F using tert-butyl 4-(3-methyl-6-oxo-5-((3-(trifluoromethyl)pyridin-2-yl)methyl)-5,6-dihydropyrido[2,3-b]pyrazin-7-yl)piperazine-1-carboxylate (50 mg, 0.1 mmol, 1 equiv) as the starting material to give the crude product 3-methyl-7-(piperazin-1-yl)-5-((3-(trifluoromethyl)pyridin-2-yl)methyl)pyrido[2,3-b]pyrazin-6(5H)-one (40 mg) as a white solid. MS m/z: 405[M+H]⁺.

Step 3: 7-(4-(2-fluoro-6-methylphenyl)piperazin-1-yl)-3-methyl-5-((3-(trifluoromethyl)pyridin-2-yl)methyl)pyrido[2,3-b]pyrazin-6(5H)-one: Followed general procedure G using 3-methyl-7-(piperazin-1-yl)-5-((3-(trifluoromethyl)pyridin-2-yl)methyl)pyrido[2,3-b]pyrazin-6(5H)-one (40 mg, 0.099 mmol, 1 equiv) and 2-bromo-1-fluoro-3-methylbenzene (22.4 mg, 0.119 mmol, 1.20 equiv) as the starting materials to give 7-(4-(2-fluoro-6-methylphenyl)piperazin-1-yl)-3-methyl-5-((3-(trifluoromethyl)pyridin-2-yl)methyl)pyrido[2,3-b]pyrazin-6(5H)-one (27.6 mg, 53.3%) as a white solid. ¹H NMR (400 MHz, Chloroform-d) δ 8.11-8.02 (m, 2H), 7.21-7.12 (m, 2H), 7.01-6.94 (m, 3H), 6.89-6.81 (m, 2H), 5.34 (s, 2H), 4.59-4.37 (m, 5H), 3.31 (t, 2H), 3.07 (d, 2H), 2.35 (s, 3H), 2.07-1.96 (m, 2H), 1.79 (d, 2H). MS m/z: 513.1 [M+H]⁺.

### 7-(1-(2-Fluoro-6-methylphenyl)piperidin-4-yl)-3-methyl-5-((3-(trifluoromethoxy)pyridin-2-yl)methyl)pyrido[2,3-b]pyrazin-6(5H)-one (68)

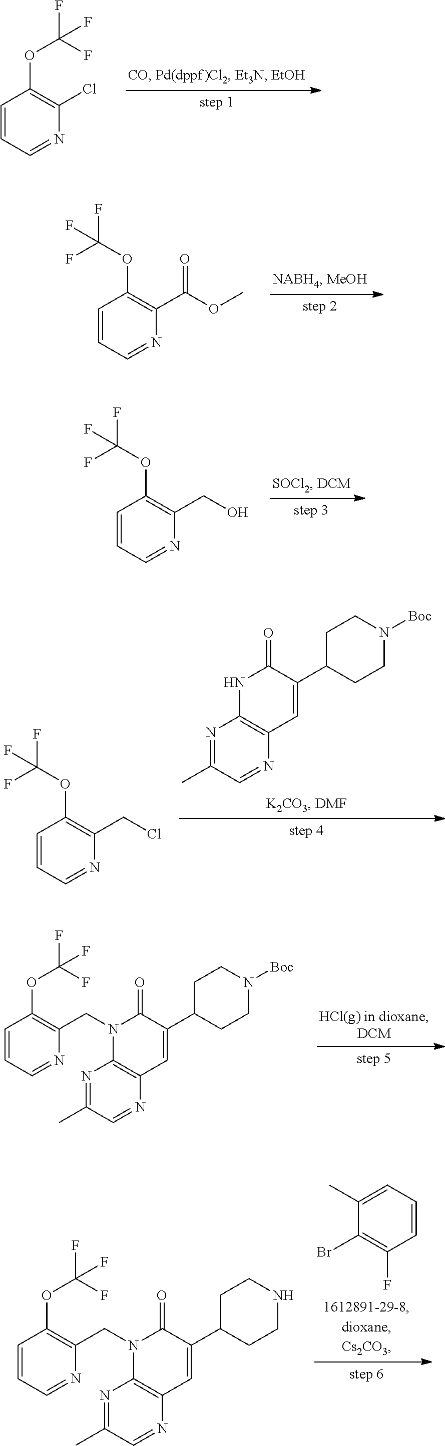

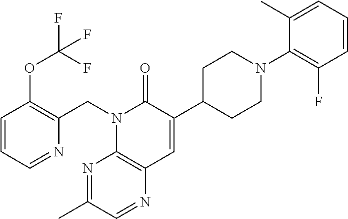 [SMILES: Cc1cnc2cc(C3CCN(c4c(C)cccc4F)CC3)c(=O)n(Cc3ncccc3OC(F)(F)F)c2n1]

Step 1: methyl 3-(trifluoromethoxy)picolinate: Followed general procedure H using 2-chloro-3-(trifluoromethoxy)pyridine (200 mg, 1.01 mmol, 1 equiv) as the starting material to give methyl 3-(trifluoromethoxy)picolinate (180 mg, 80%) as a colorless oil. MS m/z: 222 [M+H]⁺.

Step 2: (3-(trifluoromethoxy)pyridin-2-yl)methanol: Followed general procedure I using methyl 3-(trifluoromethoxy)picolinate (180 mg, 0.814 mmol, 1 equiv) as the starting material to give (3-(trifluoromethoxy)pyridin-2-yl)methanol (100 mg, 63%) as a colorless oil. MS m/z: 194 [M+H]⁺.

Step 3: 2-(chloromethyl)-3-(trifluoromethoxy)pyridine: Followed general procedure J using (3-(trifluoromethoxy)pyridin-2-yl)methanol (100 mg, 0.518 mmol, 1 equiv) as the starting material to give the crude product 2-(chloromethyl)-3-(trifluoromethoxy)pyridine (100 mg) as a white solid. MS m/z: 212 [M+H]⁺.

Step 4: tert-butyl 4-(3-methyl-6-oxo-5-((3-(trifluoromethoxy)pyridin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (90 mg, 0.261 mmol, 1 equiv) and 2-(chloromethyl)-3-(trifluoromethoxy)pyridine (82.6 mg, 0.391 mmol, 1.5 equiv) as the starting materials to give tert-butyl 4-(3-methyl-6-oxo-5-((3-(trifluoromethoxy)pyridin-2-yl)methyl)-5,6-dihydropyrido[2,3-b]pyrazin-7-yl)piperidine-1-carboxylate (70 mg, 51%) as a yellow solid. MS m/z: 520 [M+H]⁺.

Step 5: 3-methyl-7-(piperidin-4-yl)-5-((3-(trifluoromethoxy)pyridin-2-yl)methyl)pyrido[2,3-b]pyrazin-6(5H)-one: Followed general procedure F using 3-methyl-7-(piperidin-4-yl)-5-((3-(trifluoromethoxy)pyridin-2-yl)methyl)pyrido[2,3-b]pyrazin-6(5H)-one (70 mg, 0.107 mmol, 1 equiv) as the starting material to give the crude product 3-methyl-7-(piperidin-4-yl)-5-((3-(trifluoromethoxy)pyridin-2-yl)methyl)pyrido[2,3-b]pyrazin-6(5H)-one (45 mg) as an off-white solid. MS m/z: 420 [M+H]⁺.

Step 6: 7-(1-(2-fluoro-6-methylphenyl)piperidin-4-yl)-3-methyl-5-((3-(trifluoromethoxy)pyridin-2-yl)methyl)pyrido[2,3-b]pyrazin-6(5H)-one: Followed general procedure G using 3-methyl-7-(piperidin-4-yl)-5-((3-(trifluoromethoxy)pyridin-2-yl)methyl)pyrido[2,3-b]pyrazin-6(5H)-one hydrochloride (45 mg, 0.107 mmol, 1 equiv) and 3-bromo-5-fluoro-2-methylpyridine (30.4 mg, 0.161 mmol, 1.5 equiv) as the starting materials to give 7-(1-(2-fluoro-6-methylphenyl)piperidin-4-yl)-3-methyl-5-((3-(trifluoromethoxy)pyridin-2-yl)methyl)pyrido[2,3-b]pyrazin-6(5H)-one (36.3 mg, 61.3%) as an off-white solid. ¹H NMR (400 MHz, Chloroform-d) δ 8.33 (s, 1H), 8.26 (dd, 1H), 7.84 (d, 1H), 7.61-7.54 (m, 1H), 7.23-7.16 (m, 1H), 7.01-6.92 (m, 2H), 6.90-6.81 (m, 1H), 5.95 (s, 2H), 3.39-3.26 (m, 2H), 3.22-3.05 (m, 3H), 2.53 (s, 3H), 2.36 (s, 3H), 2.09-1.98 (m, 2H), 1.88-1.75 (m, 2H). MS m/z: 528.20 [M+H]⁺.

### 7-(4-(5-Fluoro-2-methylpyridin-3-yl)piperazin-1-yl)-3-methyl-5-((3-(trifluoromethyl)pyridin-2-yl)methyl)pyrido[2,3-b]pyrazin-6(5H)-one (69)

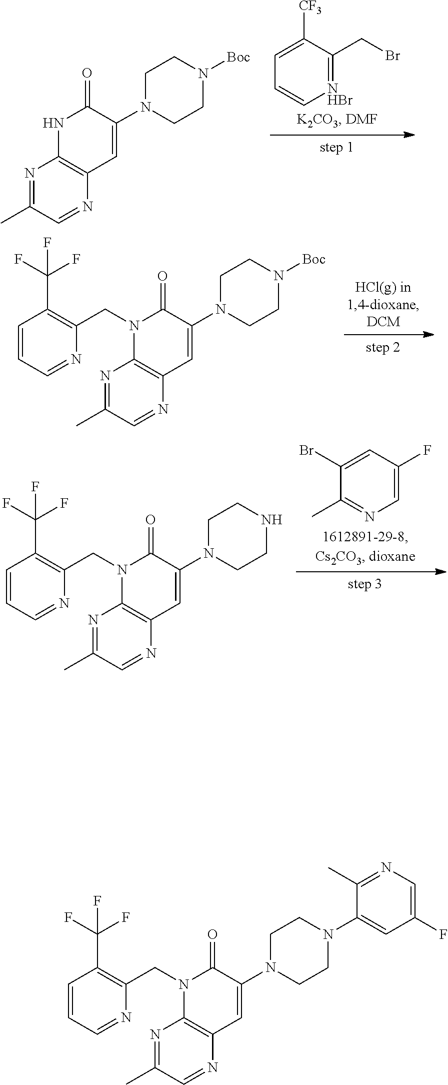

Step 1: tert-butyl 4-(3-methyl-6-oxo-5-((3-(trifluoromethyl)pyridin-2-yl)methyl)-5,6-dihydropyrido[2,3-b]pyrazin-7-yl)piperazine-1-carboxylate: Followed general procedure E using tert-butyl 4-(3-methyl-6-oxo-5,6-dihydropyrido[2,3-b]pyrazin-7-yl)piperazine-1-carboxylate (100 mg, 0.29 mmol, 1 equiv) and 2-(bromomethyl)-3-(trifluoromethyl)pyridine hydrogen bromide (92.3 mg, 0.29 mmol, 1 equiv) as the starting materials to give tert-butyl 4-(3-methyl-6-oxo-5-((3-(trifluoromethyl)pyridin-2-yl)methyl)-5,6-dihydropyrido[2,3-b]pyrazin-7-yl)piperazine-1-carboxylate (50 mg, 34%) as a yellow solid. MS m/z: 505 [M+H]⁺.

Step 2: 3-methyl-7-(piperazin-1-yl)-5-((3-(trifluoromethyl)pyridin-2-yl)methyl)pyrido[2,3-b]pyrazin-6(5H)-one: Followed general procedure F using tert-butyl 4-(3-methyl-6-oxo-5-((3-(trifluoromethyl)pyridin-2-yl)methyl)-5,6-dihydropyrido[2,3-b]pyrazin-7-yl)piperazine-1-carboxylate (50.0 mg, 0.1 mmol, 1 equiv) as the starting material to give the crude product 3-methyl-7-(piperazin-1-yl)-5-((3-(trifluoromethyl)pyridin-2-yl)methyl)pyrido[2,3-b]pyrazin-6(5H)-one (40 mg) as a white solid. MS m/z: 405[M+H]⁺.

Step 3: 7-(1-(2-fluoro-6-methylphenyl)piperidin-4-yl)-8-methyl-5-((3-(trifluoromethyl)pyridin-2-yl)methyl)pyrido[2,3-b]pyrazin-6(5H)-one: Followed general procedure G using 3-methyl-7-(piperazin-1-yl)-5-((3-(trifluoromethyl)pyridin-2-yl)methyl)pyrido[2,3-b]pyrazin-6(5H)-one (40 mg, 0.099 mmol, 1 equiv) and 3-bromo-5-fluoro-2-methylpyridine (22.6 mg, 0.119 mmol, 1.2 equiv) as the starting materials to give 7-(4-(5-fluoro-2-methylpyridin-3-yl)piperazin-1-yl)-3-methyl-5-((3-(trifluoromethyl)pyridin-2-yl)methyl)pyrido[2,3-b]pyrazin-6(5H)-one (17.6 mg, 34.2%) as a white solid. ¹H NMR (400 MHz, Chloroform-d) δ 8.43 (d, 1H), 8.29 (s, 1H), 8.12 (d, 1H), 7.97 (dd, 1H), 7.23 (d, 2H), 7.13 (dd, 1H), 6.05 (s, 2H), 3.53 (t, 4H), 3.14 (t, 4H), 2.59 (s, 3H), 2.49 (s, 3H), 1.30-1.22 (m, 1H). MS m/z: 514.10 [M+H]⁺.

### 5-((3-(Trifluoromethyl)pyrazin-2-yl)methyl)-7-(1-(4-(trifluoromethyl)pyridin-3-yl)piperidin-4-yl)pyrido[2,3-b]pyrazin-6(5H)-one (70)

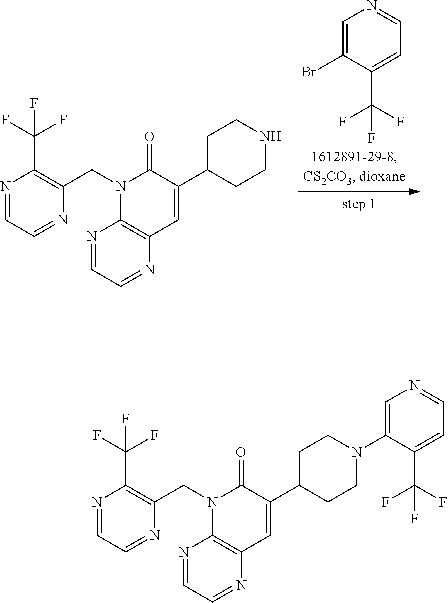 [SMILES: O=c1c(C2CCNCC2)cc2nccnc2n1Cc1nccnc1C(F)(F)F ; FC(F)(F)c1ccncc1Br ; O=c1c(C2CCN(c3cnccc3C(F)(F)F)CC2)cc2nccnc2n1Cc1nccnc1C(F)(F)F]

Step 1: 5-((3-(trifluoromethyl)pyrazin-2-yl)methyl)-7-(1-(4-(trifluoromethyl)pyridin-3-yl)piperidin-4-yl)pyrido[2,3-b]pyrazin-6(5H)-one: Followed general procedure G using 7-(piperidin-4-yl)-5-((3-(trifluoromethyl)pyrazin-2-yl)methyl)pyrido[2,3-b]pyrazin-6(5H)-one (60 mg, 0.154 mmol, 1 equiv) and 3-bromo-4-(trifluoromethyl)pyridine (52 mg, 0.231 mmol, 1.5 equiv) as the starting materials to give 5-((3-(trifluoromethyl)pyrazin-2-yl)methyl)-7-(1-(4-(trifluoromethyl)pyridin-3-yl)piperidin-4-yl)pyrido[2,3-b]pyrazin-6(5H)-one (40.9 mg, 49.6%) as a light yellow solid. ¹H NMR (400 MHz, Chloroform-d) δ 8.67 (s, 1H), 8.54-8.49 (m, 3H), 8.46-8.43 (m, 1H), 8.36 (d, 1H), 7.91 (s, 1H), 7.53 (d, 1H), 6.09 (s, 2H), 3.34 (d, 2H), 3.20-3.04 (m, 3H), 2.10 (d, 2H), 1.95-1.83 (m, 2H). MS m/z: 536.15 [M+H]⁺.

### 7-(1-(4-Chloropyridin-3-yl)piperidin-4-yl)-5-((3-(trifluoromethyl)pyridin-2-yl)methyl)-pyrido[2,3-b]pyrazin-6(5H)-one (71)

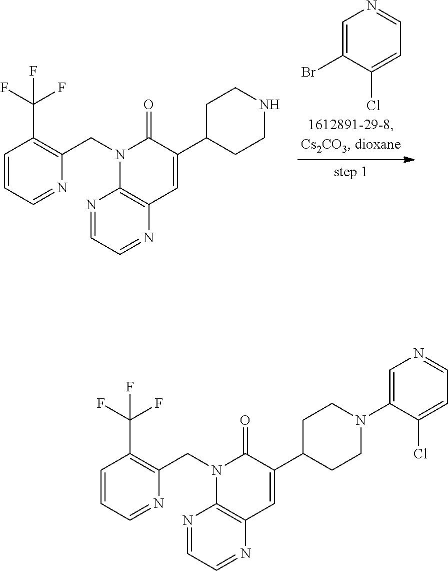 [SMILES: O=c1c(C2CCNCC2)cc2nccnc2n1Cc1ncccc1C(F)(F)F ; Clc1ccncc1Br ; O=c1c(C2CCN(c3cnccc3Cl)CC2)cc2nccnc2n1Cc1ncccc1C(F)(F)F]

Step 1: 7-(1-(4-chloropyridin-3-yl)piperidin-4-yl)-5-((3-(trifluoromethyl)pyridin-2-yl)-methyl)pyrido[2,3-b]pyrazin-6(5H)-one: Followed general procedure G using 7-(piperidin-4-yl)-5-((3-(trifluoromethyl)pyridin-2-yl)methyl)-pyrido[2,3-b]pyrazin-6(5H)-one (30 mg, 0.07 mmol, 1 equiv) and 3-bromo-4-chloropyridine (14.9 mg, 0.077 mmol, 1.1 equiv) as the starting materials to give 7-(1-(4-chloropyridin-3-yl)piperidin-4-yl)-5-((3-(trifluoromethyl)pyridin-2-yl)methyl)pyrido[2,3-b]pyrazin-6(5H)-one (10 mg, 27.1%) as an off-white solid. ¹H NMR (400 MHz, Chloroform-d) δ 8.50-8.43 (m, 1H), 8.43-8.38 (m, 1H), 8.38-8.30 (m, 1H), 8.20-8.13 (m, 1H), 7.97 (d, 1H), 7.89 (s, 1H), 7.32-7.27 (m, 1H), 7.24-7.18 (m, 2H), 6.04 (s, 2H), 3.58 (d, 2H), 3.26-3.12 (m, 1H), 2.96 (t, 2H), 2.14 (d, 2H), 1.98-1.84 (m, 2H). MS m/z: 501.1 [M+H]⁺.

### 7-(1-(2-Fluoropyridin-3-yl)piperidin-4-yl)-5-((3-(trifluoromethyl)pyridin-2-yl)methyl) pyrido[2,3-b]pyrazin-6(5H)-one (72)

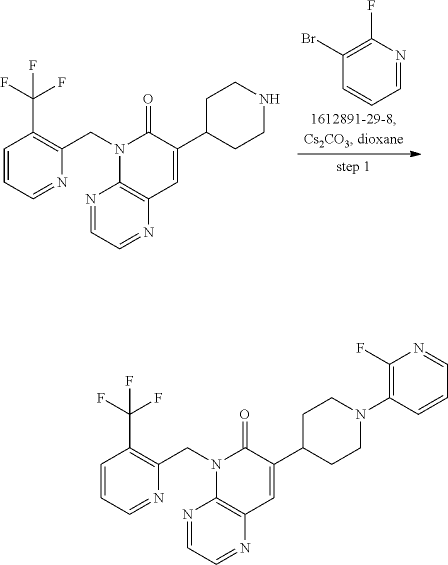 [SMILES: O=c1c(C2CCNCC2)cc2nccnc2n1Cc1ncccc1C(F)(F)F ; Fc1ncccc1Br ; O=c1c(C2CCN(c3cccnc3F)CC2)cc2nccnc2n1Cc1ncccc1C(F)(F)F]

Step 1: 7-(1-(2-fluoropyridin-3-yl)piperidin-4-yl)-5-((3-(trifluoromethyl)pyridin-2-yl)methyl)pyrido[2,3-b]pyrazin-6(5H)-one: Followed general procedure G using 7-(piperidin-4-yl)-5-((3-(trifluoromethyl)pyridin-2-yl)methyl)pyrido[2,3-b]pyrazin-6(5H)-one (40 mg, 0.094 mmol, 1 equiv) and 3-bromo-2-fluoropyridine (18.1 mg, 0.103 mmol, 1.1 equiv) as the starting materials to give 7-(1-(2-fluoropyridin-3-yl)piperidin-4-yl)-5-((3-(trifluoromethyl)pyridin-2-yl)methyl)pyrido[2,3-b]pyrazin-6(5H)-one (19.9 mg, 43.4%) as an off-white solid. ¹H NMR (400 MHz, Chloroform-d) δ 8.49 (d, 1H), 8.41 (d, 1H), 8.37 (d, 1H), 7.98 (d, 1H), 7.48 (s, 1H), 7.81 (d, 1H), 7.48 (s, 1H), 7.25-7.22 (m, 1H), 7.18-7.10 (m, 1H), 6.05 (s, 2H), 3.66 (d, 2H), 3.26-3.15 (m, 1H), 2.95 (t, 2H), 2.16 (d, 2H), 2.06-1.90 (m, 2H). MS m/z: 485.15 [M+H]⁺.

### 7-(1-(2-Fluoro-6-methylphenyl)piperidin-4-yl)-3,8-dimethyl-5-((3-(trifluoromethyl)pyrazin-2-yl)methyl)pyrido[2,3-b]pyrazin-6(5H)-one (73)

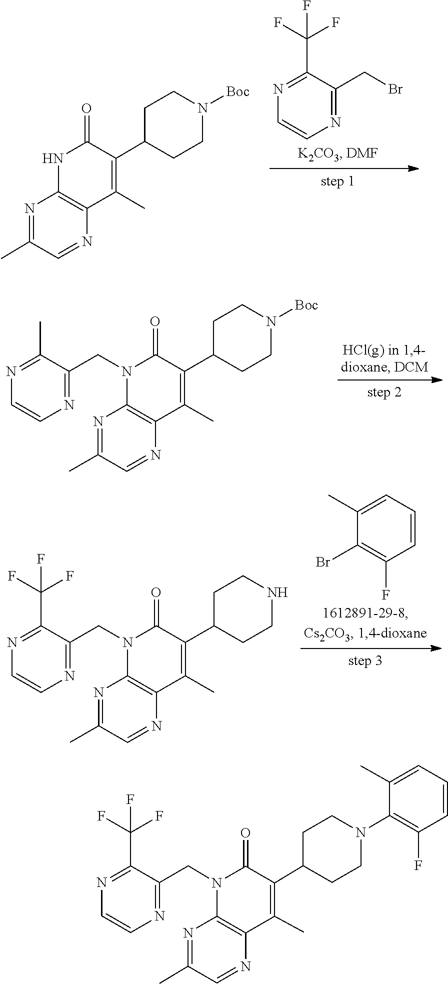

Step 1: tert-butyl 4-(3,8-dimethyl-5-((3-methylpyrazin-2-yl)methyl)-6-oxo-5,6-dihydropyrido[2,3-b]pyrazin-7-yl)piperidine-1-carboxylate: Followed general procedure E using tert-butyl 4-(3,8-dimethyl-6-oxo-5,6-dihydropyrido[2,3-b]pyrazin-7-yl)piperidine-1-carboxylate (80 mg, 0.223 mmol, 1 equiv) and 2-(bromomethyl)-3-(trifluoromethyl)pyrazine (64.5 mg, 0.268 mmol, 1.2 equiv) as the starting materials to give tert-butyl 4-(3,8-dimethyl-5-((3-methylpyrazin-2-yl)methyl)-6-oxo-5,6-dihydropyrido[2,3-b]pyrazin-7-yl)piperidine-1-carboxylate (70 mg, 60%) as a yellow oil. MS m/z: 519 [M+H]⁺.

Step 2: 3,8-dimethyl-7-(piperidin-4-yl)-5-((3-(trifluoromethyl)pyrazin-2-yl)methyl)pyrido[2,3-b]pyrazin-6(5H)-one: Followed general procedure F using tert-butyl 4-(3,8-dimethyl-5-((3-methylpyrazin-2-yl)methyl)-6-oxo-5,6-dihydropyrido[2,3-b]pyrazin-7-yl)piperidine-1-carboxylate (70 mg, 0.135 mmol, 1 equiv) as the starting material to give the crude product 3,8-dimethyl-7-(piperidin-4-yl)-5-((3-(trifluoromethyl)pyrazin-2-yl)methyl)pyrido[2,3-b]pyrazin-6(5H)-one (58 mg) as a yellow solid. MS m/z: 419 [M+H]⁺.

Step 3: 7-(1-(2-fluoro-6-methylphenyl)piperidin-4-yl)-3,8-dimethyl-5-((3-(trifluoromethyl) pyrazine-2-yl)methyl)pyrido[2,3-b]pyrazin-6(5H)-one: Followed general procedure G using 3,8-dimethyl-7-(piperidin-4-yl)-5-((3-(trifluoromethyl)pyrazin-2-yl)methyl)pyrido[2,3-b]pyrazin-6(5H)-one (58 mg, 0.139 mmol, 1 equiv) and 2-bromo-1-fluoro-3-methylbenzene (31.4 mg, 0.167 mmol, 1.2 equiv) as the starting materials to give 7-(1-(2-fluoro-6-methylphenyl)piperidin-4-yl)-3,8-dimethyl-5-((3-(trifluoromethyl)pyrazine-2-yl)methyl)pyrido[2,3-b]pyrazin-6(5H)-one (13.4 mg, 18.2%) as a white solid. ¹H NMR (400 MHz, Chloroform-d) δ 8.48-8.31 (m, 3H), 7.17-6.86 (m, 3H), 6.05 (s, 2H), 3.26 (s, 3H), 3.08 (s, 2H), 2.70 (d, 5H), 2.41 (d, 6H), 1.64-1.61 (m, 2H). MS m/z: 527.15 [M+H]⁺.

### 5-(2-(Difluoromethoxy)benzyl)-7-(1-(2-fluoro-6-methylphenyl)piperidin-4-yl)-3-methylpyrido[2,3-b]pyrazin-6(5H)-one (74)

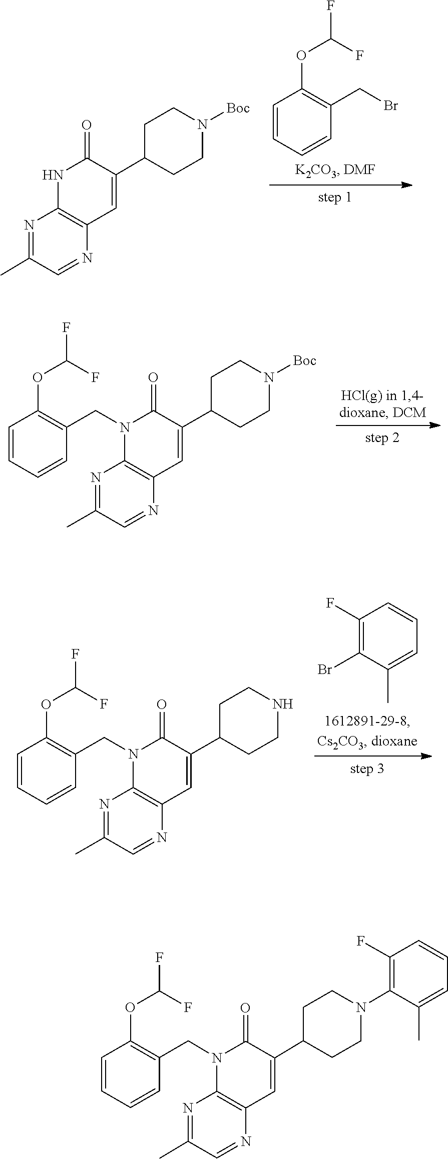

Step 1: tert-butyl 4-(5-(2-(difluoromethoxy)benzyl)-3-methyl-6-oxo-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.29 mmol, 1 equiv) and 1-(bromomethyl)-2-(difluoromethoxy)benzene (103 mg, 0.435 mmol, 1.5 equiv) as the starting materials to give tert-butyl 4-(5-(2-(difluoromethoxy)benzyl)-3-methyl-6-oxo-5,6-dihydropyrido[2,3-b]pyrazin-7-yl)piperidine-1-carboxylate (130 mg, 89%) as a white solid. MS m/z: 501 [M+H]⁺.

Step 2: 5-(2-(difluoromethoxy)benzyl)-3-methyl-7-(piperidin-4-yl)pyrido[2,3-b]pyrazin-6(5H)-one: Followed general procedure F using tert-butyl 4-(5-(2-(difluoromethoxy)benzyl)-3-methyl-6-oxo-5,6-dihydropyrido[2,3-b]pyrazin-7-yl)piperidine-1-carboxylate (130 mg, 0.260 mmol, 1 equiv) as the starting material to give the crude product 5-(2-(difluoromethoxy)benzyl)-3-methyl-7-(piperidin-4-yl)pyrido[2,3-b]pyrazin-6(5H)-one (100 mg) as a white solid. MS m/z: 401 [M+H]⁺.

Step 3: 5-(2-(difluoromethoxy)benzyl)-7-(1-(2-fluoro-6-methylphenyl)piperidin-4-yl)-3-methylpyrido[2,3-b]pyrazin-6(5H)-one: Followed general procedure G using 5-(2-(difluoromethoxy)benzyl)-3-methyl-7-(piperidin-4-yl)pyrido[2,3-b]pyrazin-6(5H)-one (100 mg, 0.25 mmol, 1 equiv) and 2-bromo-1-fluoro-3-methylbenzene (56.7 mg, 0.3 mmol, 1.2 equiv) as the starting materials to give 5-(2-(difluoromethoxy)benzyl)-7-(1-(2-fluoro-6-methylphenyl)piperidin-4-yl)-3-methylpyrido[2,3-b]pyrazin-6(5H)-one (12.3 mg, 9.33%) as a white solid. ¹H NMR (400 MHz, Chloroform-d) δ 8.35 (s, 1H), 7.83 (d, 1H), 7.24-7.21 (m, 1H), 7.18-7.12 (m, 1H), 7.10-7.02 (m, 1H), 7.00-6.95 (m, 2H), 6.94-6.89 (m, 1H), 6.88-6.50 (m, 2H), 5.80 (s, 2H), 3.36 (t, 2H), 3.14 (t, 3H), 2.58 (s, 3H), 2.38 (s, 3H), 2.03 (d, 2H), 1.85 (d, 2H). MS m/z: 509.15 [M+H]⁺.

### 7-(1-(2-Fluoro-6-methylphenyl)piperidin-4-yl)-3,8-dimethyl-5-((3-(trifluoromethyl)pyridin-2-yl)methyl)pyrido[2,3-b]pyrazin-6(5H)-one (75)

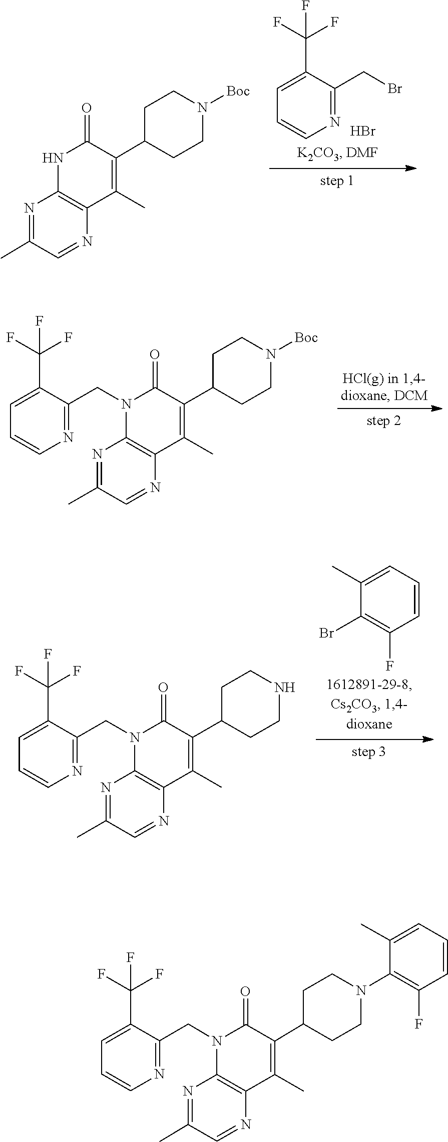

Step 1: tert-butyl 4-(3,8-dimethyl-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(3,8-dimethyl-6-oxo-5,6-dihydropyrido[2,3-b]pyrazin-7-yl)piperidine-1-carboxylate (80 mg, 0.223 mmol, 1 equiv) and 2-(bromomethyl)-3-(trifluoromethyl)pyridine hydrobromide (85.9 mg, 0.268 mmol, 1.2 equiv) as the starting materials to give tert-butyl 4-(3,8-dimethyl-6-oxo-5-((3-(trifluoromethyl)pyridin-2-yl)methyl)-5,6-dihydropyrido[2,3-b]pyrazin-7-yl)piperidine-1-carboxylate (45 mg, 38%) as a yellow oil. MS m/z: 518 [M+H]⁺.

Step 2: 3,8-dimethyl-7-(piperidin-4-yl)-5-((3-(trifluoromethyl)pyridin-2-yl)methyl)pyrido[2,3-b]pyrazin-6(5H)-one: Followed general procedure F using tert-butyl 4-(3,8-dimethyl-6-oxo-5-((3-(trifluoromethyl)pyridin-2-yl)methyl)-5,6-dihydropyrido[2,3-b]pyrazin-7-yl)piperidine-1-carboxylate (45 mg, 0.087 mmol, 1 equiv) as the starting material to give the crude product 3,8-dimethyl-7-(piperidin-4-yl)-5-((3-(trifluoromethyl)pyridin-2-yl)methyl)pyrido[2,3-b]pyrazin-6(5H)-one (35.5 mg) as a yellow oil. MS m/z: 418 [M+H]⁺.

Step 3: 7-(1-(2-fluoro-6-methylphenyl)piperidin-4-yl)-3,8-dimethyl-5-((3-(trifluoromethyl) pyridine-2-yl)methyl)pyrido[2,3-b]pyrazin-6(5H)-one: Followed general procedure G using 3,8-dimethyl-7-(piperidin-4-yl)-5-((3-(trifluoromethyl)pyridin-2-yl)methyl)pyrido[2,3-b]pyrazin-6(5H)-one (35.5 mg, 0.085 mmol, 1 equiv) and 2-bromo-1-fluoro-3-methylbenzene (19.2 mg, 0.102 mmol, 1.2 equiv) as the starting materials to give 7-(1-(2-fluoro-6-methylphenyl)piperidin-4-yl)-3,8-dimethyl-5-((3-(trifluoromethyl)pyridine-2-yl)methyl)pyrido[2,3-b]pyrazin-6(5H)-one (17.7 mg, 39.2%) as a white solid. ¹H NMR (400 MHz, Chloroform-d) δ 8.43 (d, 1H), 8.31 (s, 1H), 7.95 (d, 1H), 7.23-7.20 (m, 1H), 7.01-6.95 (m, 2H), 6.89-6.84 (m, 1H), 6.01 (s, 2H), 3.31 (s, 3H), 3.13 (s, 2H), 2.76 (s, 5H), 2.46 (s, 3H), 2.41 (s, 3H), 1.65 (d, 2H). MS m/z: 426.20 [M+H]⁺.

### 7-(1-(2-Fluoro-6-methylphenyl)piperidin-4-yl)-3-methoxy-5-((3-(trifluoromethyl)pyrazin-2-yl)methyl)pyrido[2,3-b]pyrazin-6(5H)-one (76)

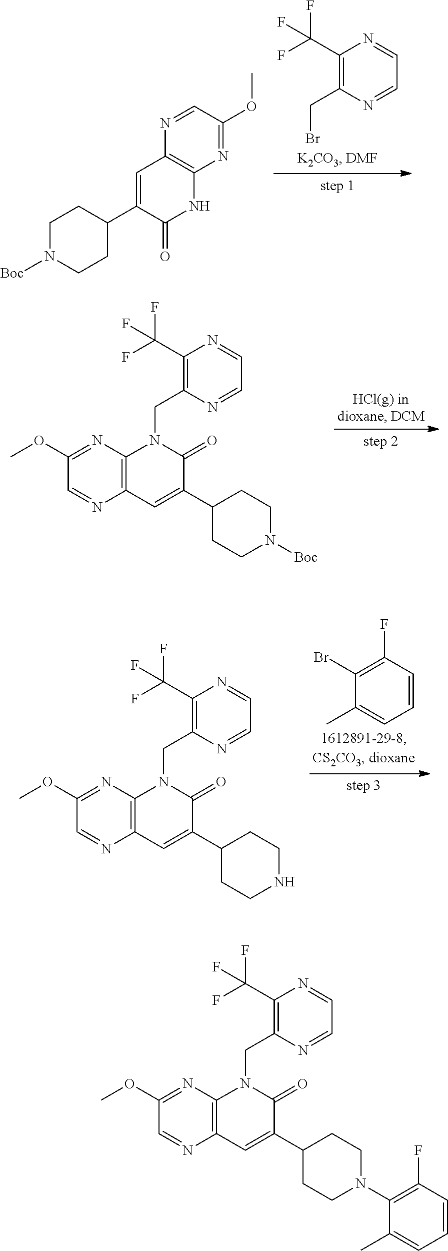

Step 1: tert-butyl 4-(3-methoxy-6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(3-methoxy-6-oxo-5,6-dihydropyrido[2,3-b]pyrazin-7-yl)piperidine-1-carboxylate (80 mg, 0.222 mmol, 1 equiv) and 2-(bromomethyl)-3-(trifluoromethyl)pyrazine (64.2 mg, 0.266 mmol, 1.2 equiv) as the starting materials to give tert-butyl 4-(3-methoxy-6-oxo-5-((3-(trifluoromethyl)pyrazin-2-yl)methyl)-5,6-dihydropyrido[2,3-b]pyrazin-7-yl)piperidine-1-carboxylate (80 mg, 69%) as a yellow oil. MS m/z: 521 [M+H]⁺.

Step 2: 3-methoxy-7-(piperidin-4-yl)-5-((3-(trifluoromethyl)pyrazin-2-yl)methyl)pyrido[2,3-b]pyrazin-6(5H)-one: Followed general procedure F using tert-butyl 4-(3-methoxy-6-oxo-5-((3-(trifluoromethyl)pyrazin-2-yl)methyl)-5,6-dihydropyrido[2,3-b]pyrazin-7-yl)piperidine-1l-carboxylate (80 mg, 0.154 mmol, 1 equiv) as the starting material to give 7-(1-(2-fluoro-6-methylphenyl)piperidin-4-yl)-3-methoxy-5-((3-(trifluoromethyl)pyrazin-2-yl)methyl)pyrido[2,3-b]pyrazin-6(5H)-one (60 mg) as an off-white solid. MS m/z: 421 [M+H]⁺.

Step 3: 7-(1-(2-fluoro-6-methylphenyl)piperidin-4-yl)-3-methoxy-5-((3-(trifluoromethyl)pyrazin-2-yl)methyl)pyrido[2,3-b]pyrazin-6(5H)-one: Followed general procedure G using 7-(1-(2-fluoro-6-methylphenyl)piperidin-4-yl)-3-methoxy-5-((3-(trifluoromethyl)pyrazin-2-yl)methyl)pyrido[2,3-b]pyrazin-6(5H)-one (45 mg, 0.107 mmol, 1 equiv) and 2-bromo-1-fluoro-3-methylbenzene (30.3 mg, 0.161 mmol, 1.5 equiv) as the starting materials to give 7-(1-(2-fluoro-6-methylphenyl)piperidin-4-yl)-3-methoxy-5-((3-(trifluoromethyl)pyrazin-2-yl)methyl)pyrido[2,3-b]pyrazin-6(5H)-one (10.2 mg, 17.5%) as a light yellow solid. ¹H NMR (400 MHz, Chloroform-d) δ 8.54-8.47 (m, 2H), 8.10 (s, 1H), 7.87 (s, 1H), 7.00-6.95 (m, 2H), 6.91-6.86 (m, 1H), 6.04 (s, 2H), 3.75 (s, 3H), 3.38 (t, 2H), 3.19-3.11 (m, 3H), 2.41 (s, 3H), 2.08-2.02 (m, 2H), 1.94-1.87 (m, 2H). MS m/z: 529.15 [M+H]⁺.

### 5-((3-Fluoropyrazin-2-yl)methyl)-8-methyl-7-(1-(2-(trifluoromethyl)phenyl)piperidin-4-yl)pyrido[2,3-b]pyrazin-6(5H)-one (77)

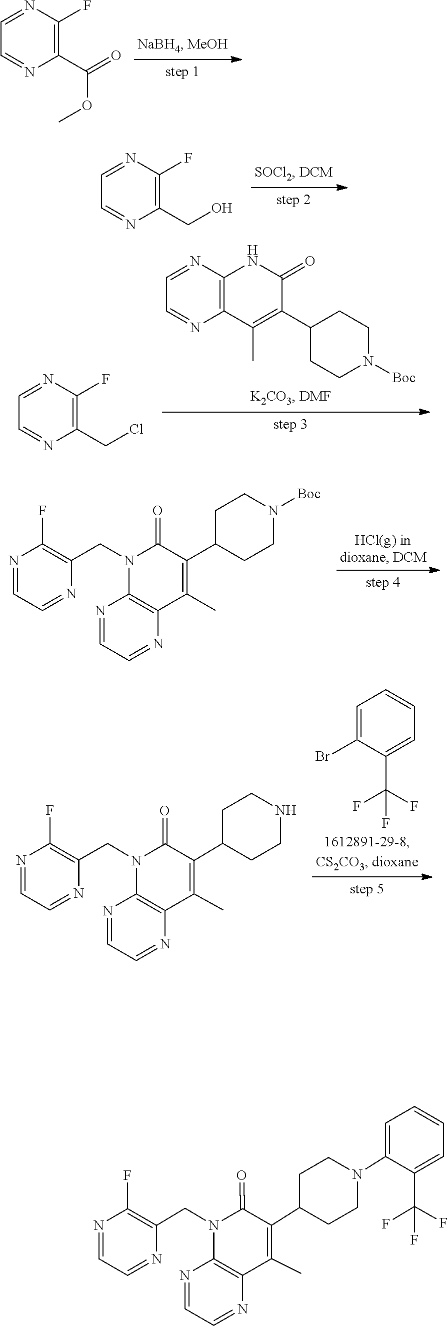

Step 1: (3-fluoropyrazin-2-yl)methanol: Followed general procedure I using methyl 3-fluoropyrazine-2-carboxylate (300 mg, 1.92 mmol, 1 equiv) as the starting material to give (3-fluoropyrazin-2-yl)methanol (80 mg, 32%) as a yellow oil. MS m/z: 129 [M+H]⁺.

Step 2: 2-(chloromethyl)-3-fluoropyrazine: Followed general procedure J using (3-fluoropyrazin-2-yl)methanol (80 mg, 0.625 mmol, 1 equiv) as the starting material to give the crude product 2-(chloromethyl)-3-fluoropyrazine (40 mg) as a yellow oil. MS m/z: 147 [M+H]⁺.

Step 3: tert-butyl 4-(5-((3-fluoropyrazin-2-yl)methyl)-8-methyl-6-oxo-5,6-dihydropyrido[2,3-b]pyrazin-7-yl)piperidine-1-carboxylate: Followed general procedure E using tert-butyl 4-(8-methyl-6-oxo-5,6-dihydropyrido[2,3-b]pyrazin-7-yl)piperidine-1-carboxylate (94 mg, 0.27 mmol, 1 equiv) and 2-(chloromethyl)-3-fluoropyrazine (40 mg, 0.27 mmol, 1 equiv) as the starting materials to give tert-butyl 4-(5-((3-fluoropyrazin-2-yl)methyl)-8-methyl-6-oxo-5,6-dihydropyrido[2,3-b]pyrazin-7-yl)piperidine-1-carboxylate (40 mg, 32%) as a yellow oil. MS m/z: 455 [M+H]⁺.

Step 4: 5-((3-fluoropyrazin-2-yl)methyl)-8-methyl-7-(piperidin-4-yl)pyrido[2,3-b]pyrazin-6(5H)-one: Followed general procedure F using tert-butyl 4-(5-((3-fluoropyrazin-2-yl)methyl)-8-methyl-6-oxo-5,6-dihydropyrido[2,3-b]pyrazin-7-yl)piperidine-1-carboxylate (40 mg, 0.088 mmol, 1 equiv) as the starting material to give the crude product 5-((3-fluoropyrazin-2-yl)methyl)-8-methyl-7-(piperidin-4-yl)pyrido[2,3-b]pyrazin-6(5H)-one (30 mg) as a yellow oil. MS m/z: 355 [M+H]⁺.

Step 5: 5-((3-fluoropyrazin-2-yl)methyl)-8-methyl-7-(1-(2-(trifluoromethyl)phenyl)piperidin-4-yl)pyrido[2,3-b]pyrazin-6(5H)-one: Followed general procedure G using 5-((3-fluoropyrazin-2-yl)methyl)-8-methyl-7-(piperidin-4-yl)pyrido[2,3-b]pyrazin-6(5H)-one (30 mg, 0.077 mmol, 1 equiv) and 2-bromo-1-fluoro-3-methylbenzene (17.3 mg, 0.077 mmol, 1 equiv) as the starting materials to give 5-((3-fluoropyrazin-2-yl)methyl)-8-methyl-7-(1-(2-(trifluoromethyl) phenyl) piperidin-4-yl)pyrido[2,3-b]pyrazin-6(5H)-one (16.9 mg, 44.2%) as a white solid. ¹H NMR (300 MHz, Chloroform-d) δ 8.51 (d, 1H), 8.35 (d, 1H), 8.25-8.20 (m, 1H), 8.08-8.04 (m, 1H), 7.65 (d, 1H), 7.54 (t, 1H), 7.43 (d, 1H), 7.23 (t, 1H), 5.94 (s, 2H), 3.54-3.40 (m, 1H), 3.24 (d, 2H), 2.91 (t, 2H), 2.80 (s, 3H), 2.77-2.60 (m, 2H), 1.70 (d, 2H). MS m/z: 499.15 [M+H]⁺.

### 7-(1-(2-Fluoro-6-methylphenyl)piperidin-4-yl)-3-methyl-5-(2-(trifluoromethoxy)benzyl)pyrido[2,3-b]pyrazin-6(5H)-one (78)

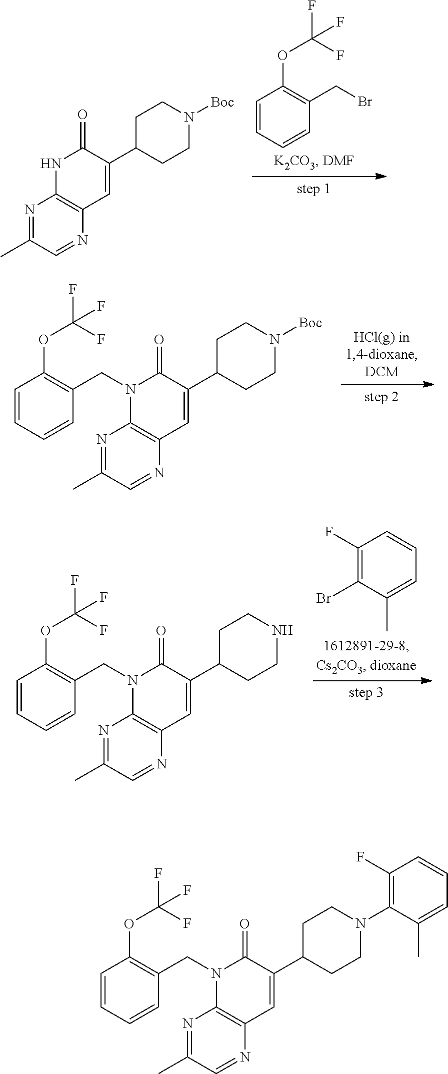

Step 1: tert-butyl 4-(3-methyl-6-oxo-5-(2-(trifluoromethoxy)benzyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.29 mmol, 1 equiv) and 1-(bromomethyl)-2-(trifluoromethoxy)benzene (103 mg, 0.435 mmol, 1.5 equiv) as the starting materials to give tert-butyl 4-(3-methyl-6-oxo-5-(2-(trifluoromethoxy)benzyl)-5,6-dihydropyrido[2,3-b]pyrazin-7-yl)piperidine-1-carboxylate (130 mg, 89%) as a white solid. MS m/z: 519 [M+H]⁺.

Step 2: 3-methyl-7-(piperidin-4-yl)-5-(2-(trifluoromethoxy)benzyl)pyrido[2,3-b]pyrazin-6(5H)-one: Followed general procedure F using tert-butyl 4-(3-methyl-6-oxo-5-(2-(trifluoromethoxy)benzyl)-5,6-dihydropyrido[2,3-b]pyrazin-7-yl)piperidine-1-carboxylate (130 mg, 0.26 mmol, 1 equiv) as the starting material to give the crude product 3-methyl-7-(piperidin-4-yl)-5-(2-(trifluoromethoxy)benzyl)pyrido[2,3-b]pyrazin-6(5H)-one (100 mg) as a white solid. MS m/z: 419 [M+H]⁺.

Step 3: 7-(1-(2-fluoro-6-methylphenyl)piperidin-4-yl)-3-methyl-5-(2-(trifluoromethoxy)benzyl)pyrido[2,3-b]pyrazin-6(5H)-one: Followed general procedure G using 3-methyl-7-(piperidin-4-yl)-5-(2-(trifluoromethoxy)benzyl)pyrido[2,3-b]pyrazin-6(5H)-one (100 mg, 0.25 mmol, 1 equiv) and 2-bromo-1-fluoro-3-methylbenzene (56.7 mg, 0.3 mmol, 1.2 equiv) as the starting materials to give 7-(1-(2-fluoro-6-methylphenyl)piperidin-4-yl)-3-methyl-5-(2-(trifluoromethoxy)benzyl)pyrido[2,3-b]pyrazin-6(5H)-one (12.3 mg, 9.3%) as a white solid. ¹H NMR (400 MHz, Chloroform-d) δ 8.36 (s, 1H), 7.87 (d, 1H), 7.25 (s, 2H), 7.18-7.09 (m, 1H), 7.07-6.96 (m, 3H), 6.94-6.86 (m, 1H), 5.81 (s, 2H), 3.48 (d, 2H), 3.24 (d, 3H), 2.57 (s, 3H), 2.48 (s, 3H), 2.07 (s, 4H). MS m/z: 527.10 [M+H]⁺.

### 7-(1-(2-Fluoro-4-methoxypyridin-3-yl)piperidin-4-yl)-5-((3-(trifluoromethyl)pyridin-2-yl)methyl)pyrido[2,3-b]pyrazin-6(5H)-one (79)

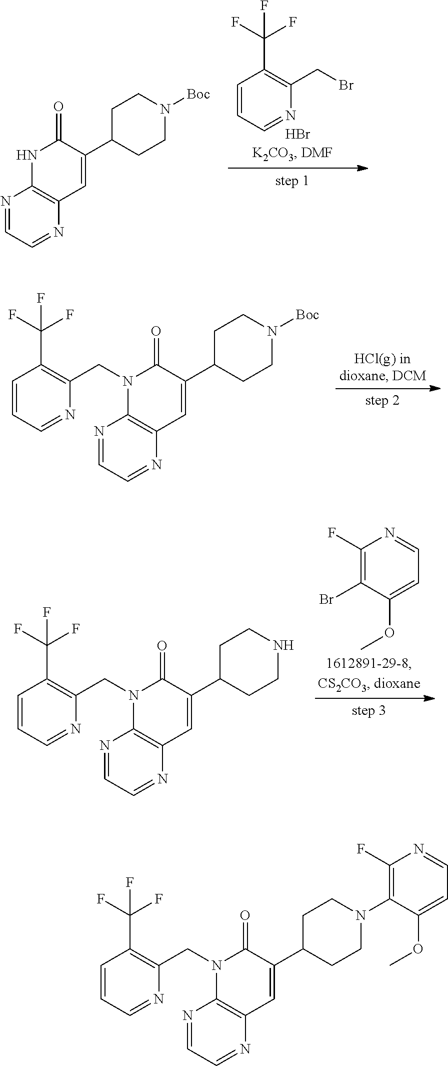

Step 1: tert-butyl 4-(6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (90 mg, 0.272 mmol, 1 equiv) and 2-(bromomethyl)-3-(trifluoromethyl)pyridine hydrobromide (83 mg, 0.258 mmol, 0.95 equiv) as the starting materials to give tert-butyl 4-(6-oxo-5-((3-(trifluoromethyl)pyridin-2-yl)methyl)-5,6-dihydropyrido[2,3-b]pyrazin-7-yl)piperidine-1-carboxylate (85 mg, 63%) as a yellow oil. MS m/z: 490 [M+H]⁺.

Step 2: 7-(piperidin-4-yl)-5-((3-(trifluoromethyl)pyridin-2-yl)methyl)pyrido[2,3-b]pyrazin-6(5H)-one: Followed general procedure F using tert-butyl 4-(6-oxo-5-((3-(trifluoromethyl)pyridin-2-yl)methyl)-5,6-dihydropyrido[2,3-b]pyrazin-7-yl)piperidine-1-carboxylate (85 mg, 0.174 mmol, 1 equiv) as the starting material to give crude product 7-(piperidin-4-yl)-5-((3-(trifluoromethyl)pyridin-2-yl)methyl)pyrido[2,3-b]pyrazin-6(5H)-one (60 mg) as a yellow oil. MS m/z: 390 [M+H]⁺.

Step 3: 7-(1-(2-fluoro-4-methoxypyridin-3-yl)piperidin-4-yl)-5-((3-(trifluoromethyl)pyridin-2-yl)methyl)pyrido[2,3-b]pyrazin-6(5H)-one: Followed general procedure G using tert-butyl 4-(8-methyl-6-oxo-5-((3-(trifluoromethyl)pyridin-2-yl)methyl)-5,6-dihydropyrido[2,3-b]pyrazin-7-yl)piperazine-1-carboxylate (50 mg, 0.128 mmol, 1 equiv) and 3-bromo-2-fluoro-4-methoxypyridine (31.7 mg, 0.154 mmol, 1.2 equiv) as the starting materials to give 7-(1-(2-fluoro-4-methoxypyridin-3-yl)piperidin-4-yl)-5-((3-(trifluoromethyl)pyridin-2-yl)methyl)pyrido[2,3-b]pyrazin-6(5H)-one (8 mg, 11.4%) as an off-white solid. ¹H NMR (400 MHz, Chloroform-d) δ 8.47 (d, 1H), 8.43-8.40 (m, 1H), 8.36 (d, 1H), 7.97 (d, 1H), 7.89 (s, 1H), 7.85 (d, 1H), 7.24-7.22 (m, 1H), 6.73 (d, 1H), 6.04 (s, 2H), 3.95 (s, 3H), 3.36-3.28 (m, 4H), 3.22-3.15 (m, 1H), 2.07-1.93 (m, 4H). MS m/z: 515.10 [M+H]⁺.

### 7-(1-(2-Fluoro-4-methoxypyridin-3-yl)piperidin-4-yl)-3-methyl-5-((3-(trifluoromethyl)pyrazin-2-yl)methyl)pyrido[2,3-b]pyrazin-6(5H)-one (80)

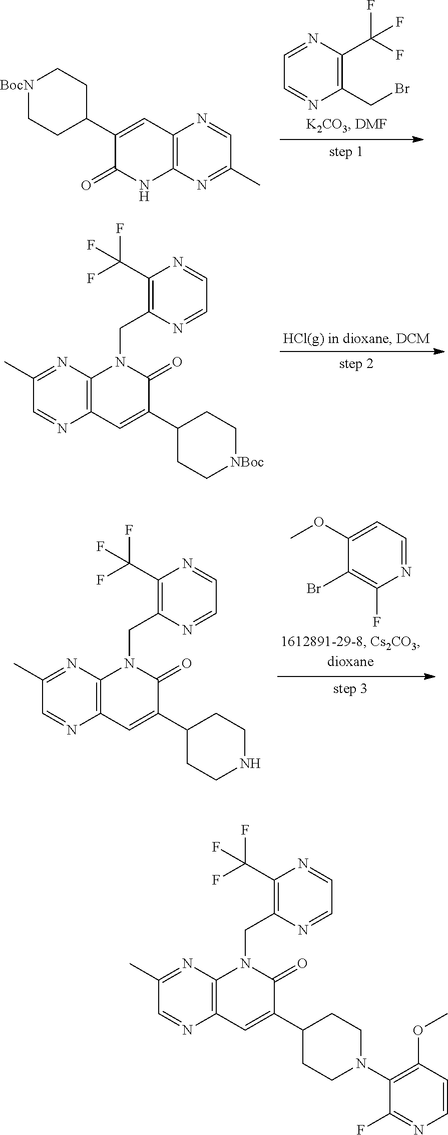

Step 1: tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.29 mmol, 1 equiv) and 2-(bromomethyl)-3-(trifluoromethyl)pyrazine (104 mg, 0.435 mmol, 1.5 equiv) as the starting materials to give tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (90 mg, 61%) as an off-white solid. MS m/z: 505 [M+H]⁺.

Step 2: 3-methyl-7-(piperidin-4-yl)-5-((3-(trifluoromethyl)pyrazin-2-yl)methyl)pyrido[2,3-b]pyrazin-6(5H)-one: Followed general procedure F using tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (90 mg, 0.178 mmol, 1 equiv) as the starting material to give the crude product 3-methyl-7-(piperidin-4-yl)-5-((3-(trifluoromethyl)pyrazin-2-yl)methyl)pyrido[2,3-b]pyrazin-6(5H)-one (60 mg) as an off-white solid. MS m/z: 405 [M+H]⁺.

Step 3: 7-(1-(2-fluoro-4-methoxypyridin-3-yl)piperidin-4-yl)-3-methyl-5-((3-(trifluoromethyl)pyrazin-2-yl)methyl)pyrido[2,3-b]pyrazin-6(5H)-one: Followed general procedure G using 3-methyl-7-(piperidin-4-yl)-5-((3-(trifluoromethyl)pyrazin-2-yl)methyl)pyrido[2,3-b]pyrazin-6(5H)-one (60 mg, 0.148 mmol, 1 equiv) and 3-bromo-2-fluoro-4-methoxypyridine (36.6 mg, 0.178 mmol, 1.2 equiv) as the starting materials to give 7-(1-(2-fluoro-4-methoxypyridin-3-yl)piperidin-4-yl)-3-methyl-5-((3-(trifluoromethyl)pyrazin-2-yl)methyl)pyrido[2,3-b]pyrazin-6(5H)-one (9.8 mg, 11.9%) as an off-white solid. ¹H NMR (400 MHz, Chloroform-d) δ 8.48 (dd, 2H), 8.35 (s, 1H), 7.90-7.80 (m, 2H), 6.73 (d, 1H), 6.07 (s, 2H), 3.94 (s, 3H), 3.39-3.27 (m, 4H), 3.21-3.09 (m, 1H), 2.51 (s, 3H), 2.10-1.99 (m, 2H), 1.98-1.86 (m, 2H). MS m/z: 530.15 [M+H]⁺.

### 6-(1-(2-Fluoro-6-methylphenyl)piperidin-4-yl)-2-methyl-8-((3-(trifluoromethyl)253yridine-2-yl)methyl)pyrido[2,3-d]pyrimidin-7(8H)-one (81)

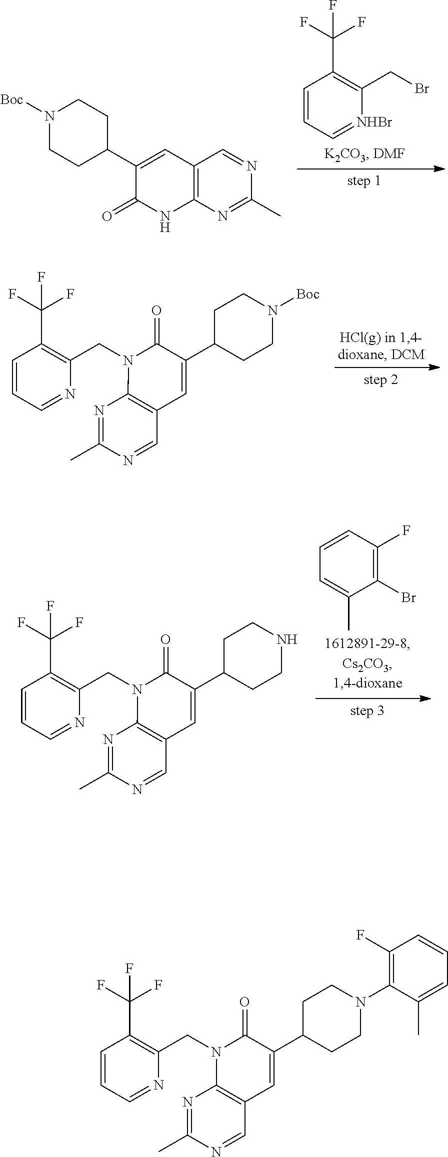

Step 1: tert-butyl 4-(2-methyl-7-oxo-8-((3-(trifluoromethyl)pyridin-2-yl)methyl)-7,8-dihydropyrido[2,3-d]pyrimidin-6-yl)piperidine-1-carboxylate: Followed general procedure E using tert-butyl 4-(2-methyl-7-oxo-7,8-dihydropyrido[2,3-d]pyrimidin-6-yl)piperidine-1-carboxylate (80 mg, 0.232 mmol, 1 equiv) and (2-(bromomethyl)-3-(trifluoromethyl)pyridine hydrobromide (82 mg, 0.255 mmol, 1.1 equiv) as the starting materials to give tert-butyl 4-(2-methyl-7-oxo-8-((3-(trifluoromethyl)pyridin-2-yl) methyl)-7,8-dihydropyrido[2,3-d]pyrimidin-6-yl)piperidine-1-carboxylate (78.6 mg, 67%) as a colorless oil. MS m/z: 504 [M+H]⁺.

Step 2: 2-methyl-6-(piperidin-4-yl)-8-((3-(trifluoromethyl)pyridin-2-yl)methyl)pyrido[2,3-d]pyrimidin-7(8H)-one: Followed general procedure F using tert-butyl 4-(2-methyl-7-oxo-8-((3-(trifluoromethyl)pyridin-2-yl)methyl)-7,8-dihydropyrido[2,3-d]pyrimidin-6-yl)piperidine-1-carboxylate (78.6 mg, 0.156 mmol, 1 equiv) as the starting material to give the crude product 2-methyl-6-(piperidin-4-yl)-8-((3-(trifluoromethyl)pyridin-2-yl)methyl)pyrido[2,3-d]pyrimidin-7(8H)-one (60 mg) as a light yellow solid. MS m/z: 404 [M+H]⁺.

Step 3: 6-(1-(2-fluoro-6-methylphenyl)piperidin-4-yl)-2-methyl-8-((3-(trifluoromethyl)pyridin-2-yl)methyl)pyrido[2,3-d]pyrimidin-7(8H)-one: Followed general procedure G using 2-methyl-6-(piperidin-4-yl)-8-((3-(trifluoromethyl)pyridin-2-yl)methyl)pyrido[2,3-d]pyrimidin-7(8H)-one (60 mg, 0.149 mmol, 1 equiv) and 2-bromo-1-fluoro-3-methylbenzene (33.7 mg, 0.179 mmol, 1.2 equiv) as the starting materials to give 6-(1-(2-fluoro-6-methylphenyl)piperidin-4-yl)-2-methyl-8-((3-(trifluoromethyl) pyridin-2-yl)methyl)pyrido[2,3-d]pyrimidin-7(8H)-one (31.0 mg, 40.1%) as a white solid. ¹H NMR (400 MHz, Chloroform-d) δ 8.80 (s, 1H), 8.44-8.43 (m, 1H), 7.99-7.96 (m, 1H), 7.60 (s, 1H), 7.25-7.23 (m, 1H), 7.00-6.94 (m, 2H), 6.90-6.83 (m, 1H), 6.01 (s, 2H), 3.35-3.28 (m, 2H), 3.14-3.07 (m, 3H), 2.67 (s, 3H), 2.36 (s, 3H), 2.04-1.99 (m, 2H), 1.82-1.73 (m, 2H). MS m/z: 512.20 [M+H]⁺.

### 7-(1-(2-Fluoro-6-methylphenyl)piperidin-4-yl)-5-((3-(trifluoromethyl)pyrazin-2-yl)methyl)pyrido[3,2-d]pyrimidin-6(5H)-one (82)

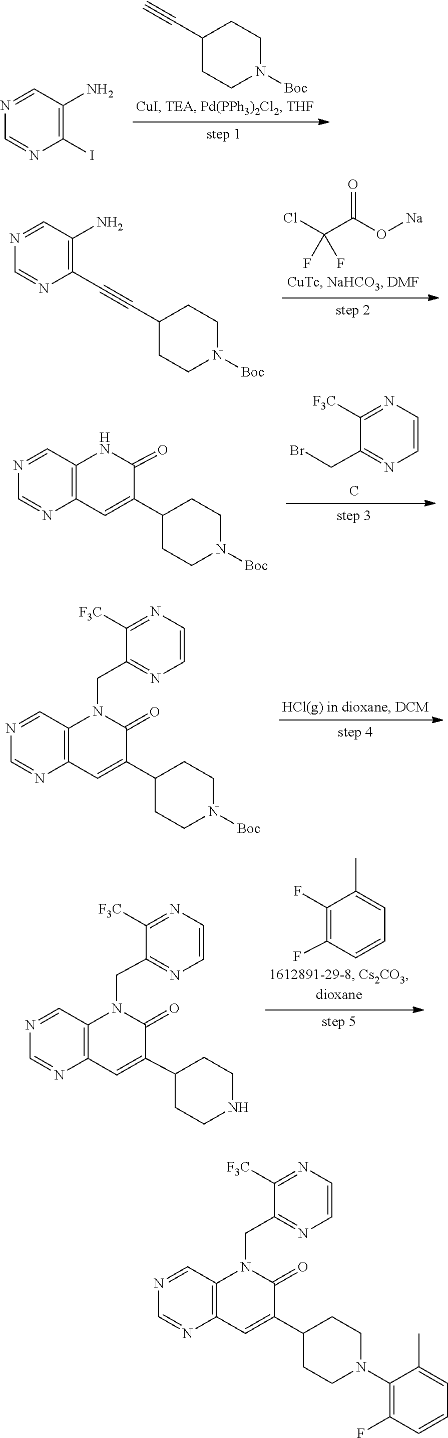

Step 1: tert-butyl 4-((5-aminopyrimidin-4-yl)ethynyl)piperidine-1-carboxylate: Followed general procedure Z using 4-iodopyrimidin-5-amine (200 mg, 0.905 mmol, 1 equiv) and tert-butyl 4-ethynylpiperidine-1-carboxylate (284 mg, 1.35 mmol, 1.5 equiv) as the starting materials to give tert-butyl 4-[2-(5-aminopyrimidin-4-yl)ethynyl]piperidine-1-carboxylate (200 mg, 73%) as a white solid. MS m/z: 303 [M+H]⁺.

Step 2: tert-butyl 4-(6-oxo-5,6-dihydropyrido[3,2-d]pyrimidin-7-yl)piperidine-1-carboxylate: Followed general procedure AA using tert-butyl 4-((5-aminopyrimidin-4-yl)ethynyl)piperidine-1-carboxylate (150 mg, 0.496 mmol, 1 equiv) and sodium 2-chloro-2,2-difluoroacetate (151 mg, 0.992 mmol, 2 equiv) as the starting materials to give tert-butyl 4-(6-oxo-5,6-dihydropyrido[3,2-d]pyrimidin-7-yl)piperidine-1-carboxylate (50 mg, 30%) as a white solid. MS m/z: 331 [M+H]⁺.

Step 3: tert-butyl 4-(6-oxo-5-((3-(trifluoromethyl)pyrazin-2-yl)methyl)-5,6-dihydropyrido[3,2-d]pyrimidin-7-yl)piperidine-1-carboxylate: Followed general procedure E using tert-butyl 4-(6-oxo-5,6-dihydropyrido[3,2-d]pyrimidin-7-yl)piperidine-1-carboxylate (50 mg, 0.151 mmol, 1 equiv) and 2-(bromomethyl)-3-(trifluoromethyl)pyrazine (36.2 mg, 0.151 mmol, 1 equiv) as the starting materials to give tert-butyl 4-(6-oxo-5-((3-(trifluoromethyl)pyrazin-2-yl)methyl)-5,6-dihydropyrido[3,2-d]pyrimidin-7-yl)piperidine-1-carboxylate (50 mg, 67%) as a white solid. MS m/z: 491 [M+H]⁺.

Step 4: 7-(piperidin-4-yl)-5-((3-(trifluoromethyl)pyrazin-2-yl)methyl)pyrido[3,2-d]pyrimidin-6(5H)-one: Followed general procedure F using tert-butyl 4-(6-oxo-5-((3-(trifluoromethyl)pyrazin-2-yl)methyl)-5,6-dihydropyrido[3,2-d]pyrimidin-7-yl)piperidine-1-carboxylate (50 mg, 0.102 mmol, 1 equiv) as the starting material to give the crude product 7-(piperidin-4-yl)-5-((3-(trifluoromethyl)pyrazin-2-yl)methyl)pyrido[3,2-d]pyrimidin-6(5H)-one (40 mg) as a yellow oil. MS m/z: 391 [M+H]⁺.

Step 5: 7-(1-(2-fluoro-6-methylphenyl)piperidin-4-yl)-5-((3-(trifluoromethyl)pyrazin-2-yl)methyl)pyrido[3,2-d]pyrimidin-6(5H)-one: Followed general procedure G using 7-(piperidin-4-yl)-5-((3-(trifluoromethyl)pyrazin-2-yl)methyl)pyrido[3,2-d]pyrimidin-6(5H)-one (30 mg, 0.077 mmol, 1 equiv) and 2-bromo-1-fluoro-3-methylbenzene (21.7 mg, 0.115 mmol, 1.5 equiv) as the starting materials to give 7-(1-(2-fluoro-6-methylphenyl)piperidin-4-yl)-5-((3-(trifluoromethyl)pyrazin-2-yl)methyl)pyrido[3,2-d]pyrimidin-6(5H)-one (11.8 mg, 29.6%) as a yellow solid. ¹H NMR (400 MHz, Chloroform-d) δ 9.23 (d, 2H), 8.76 (d, 1H), 8.67 (d, 1H), 8.13 (s, 1H), 7.01-6.94 (m, 2H), 6.91-6.84 (m, 1H), 5.96 (s, 2H), 3.35-3.25 (m, 2H), 3.24-3.15 (m, 1H), 3.15-3.06 (m, 2H), 2.35 (s, 3H), 2.13-2.05 (m, 2H), 1.95-1.83 (m, 2H). MS m/z: 499.10 [M+H]⁺.

### 5-((3-Fluoropyridin-2-yl)methyl)-8-methyl-7-(1-(2-(trifluoromethyl)phenyl)piperidin-4-yl)pyrido[2,3-b]pyrazin-6(5H)-one (83)

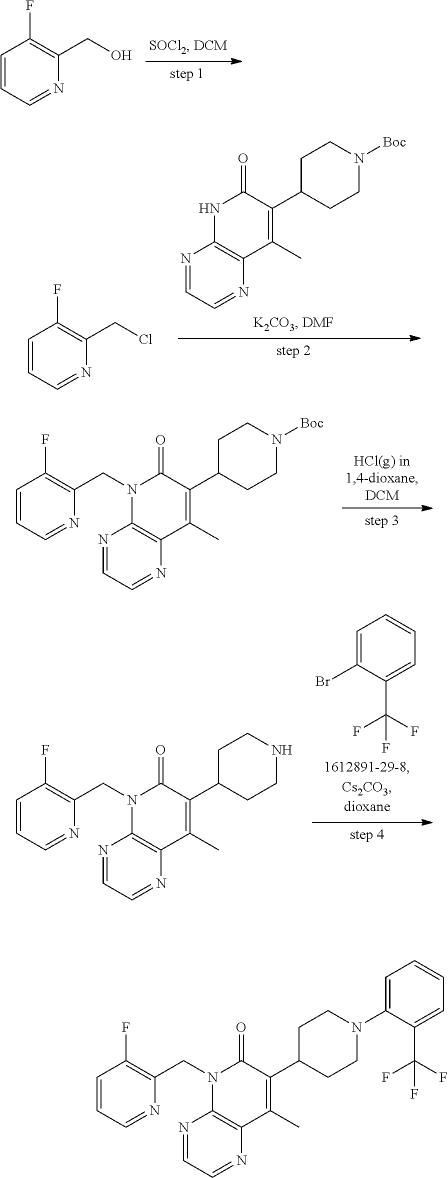

Step 1: 2-(chloromethyl)-3-fluoropyridine: Followed general procedure J using (3-fluoropyridin-2-yl)methanol (300 mg, 2.36 mmol, 1 equiv) as the starting material to give the crude product 2-(chloromethyl)-3-fluoropyridine (300 mg, 87%) as a white solid. MS m/z: 146 [M+H]⁺.

Step 2: tert-butyl 4-(5-((3-fluoropyridin-2-yl)methyl)-8-methyl-6-oxo-5,6-dihydropyrido[2,3-b]pyrazin-7-yl)piperidine-1-carboxylate: Followed general procedure E using 2-(chloromethyl)-3-fluoropyridine (100 mg, 0.687 mmol, 1 equiv) and tert-butyl 4-(8-methyl-6-oxo-5,6-dihydropyrido[2,3-b]pyrazin-7-yl)piperidine-1-carboxylate (236 mg, 0.687 mmol, 1.5 equiv) as the starting materials to give tert-butyl 4-(5-((3-fluoropyridin-2-yl)methyl)-8-methyl-6-oxo-5,6-dihydropyrido[2,3-b]pyrazin-7-yl)piperidine-1-carboxylate (100 mg, 32%) as a white solid. MS m/z: 454 [M+H]⁺.

Step 3: 5-((3-fluoropyridin-2-yl)methyl)-8-methyl-7-(piperidin-4-yl)pyrido[2,3-b]pyrazin-6(5H)-one: Followed general procedure F using tert-butyl 4-(5-((3-fluoropyridin-2-yl)methyl)-8-methyl-6-oxo-5,6-dihydropyrido[2,3-b]pyrazin-7-yl)piperidine-1-carboxylate (100 mg, 0.22 mmol, 1 equiv) as the starting material to give the crude product 5-((3-fluoropyridin-2-yl)methyl)-8-methyl-7-(piperidin-4-yl)pyrido[2,3-b]pyrazin-6(5H)-one (80 mg) as a white solid. MS m/z: 354 [M+H]⁺.

Step 4: 5-((3-fluoropyridin-2-yl)methyl)-8-methyl-7-(1-(2-(trifluoromethyl)phenyl)piperidin-4-yl)pyrido[2,3-b]pyrazin-6(5H)-one: Followed general procedure G using 5-((3-fluoropyridin-2-yl)methyl)-8-methyl-7-(piperidin-4-yl)pyrido[2,3-b]pyrazin-6(5H)-one (80 mg, 0.226 mmol, 1 equiv) and 1-bromo-2-(trifluoromethyl)benzene (61.1 mg, 0.271 mmol, 1.2 equiv) as the starting materials to 5-((3-fluoropyridin-2-yl)methyl)-8-methyl-7-(1-(2-(trifluoromethyl)phenyl)piperidin-4-yl)pyrido[2,3-b]pyrazin-6(5H)-one (63.9 mg, 56.5%) as a white solid. ¹H NMR (400 MHz, Chloroform-d) δ 8.47 (d, 1H), 8.35 (d, 1H), 8.19-8.12 (m, 1H), 7.66-7.59 (m, 1H), 7.55-7.46 (m, 1H), 7.43-7.36 (m, 2H), 7.21-7.13 (m, 2H), 5.92 (d, 2H), 3.52-3.41 (m, 1H), 3.20 (d, 2H), 2.93-2.82 (m, 2H), 2.77 (s, 3H), 2.72-2.58 (m, 3H), 1.68 (d, 2H). MS m/z: 498.05 [M+H]⁺.

### 7-(1-(2-Fluoro-6-methylphenyl)piperidin-4-yl)-5-((3-(trifluoromethyl)pyridin-2-yl)methyl)pyrido[3,2-d]pyrimidin-6(5H)-one (84)

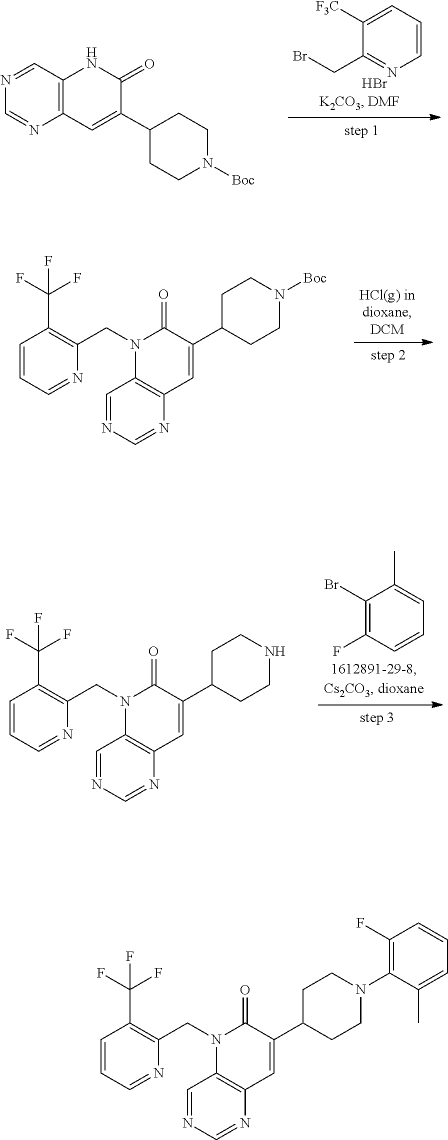

Step 1: tert-butyl 4-(6-oxo-5-((3-(trifluoromethyl)pyridin-2-yl)methyl)-5,6-dihydropyrido[3,2-d]pyrimidin-7-yl)piperidine-1-carboxylate: Followed general procedure E using 2-(bromomethyl)-3-(trifluoromethyl)pyridine hydrobromide (86.7 mg, 0.272 mmol, 1 equiv) and tert-butyl 4-(6-oxo-5,6-dihydropyrido[3,2-d]pyrimidin-7-yl)piperidine-1-carboxylate (90 mg, 0.272 mmol, 1.5 equiv) as the starting materials to give tert-butyl 4-(6-oxo-5-((3-(trifluoromethyl)pyridin-2-yl)methyl)-5,6-dihydropyrido[3,2-d]pyrimidin-7-yl)piperidine-1-carboxylate (50 mg, 37%) as a white solid. MS m/z: 490 [M+H]⁺.

Step 2: 7-(piperidin-4-yl)-5-((3-(trifluoromethyl)pyridin-2-yl)methyl)pyrido[3,2-d]pyrimidin-6(5H)-one: Followed general procedure F using tert-butyl 4-(6-oxo-5-((3-(trifluoromethyl)pyridin-2-yl)methyl)-5,6-dihydropyrido[3,2-d]pyrimidin-7-yl)piperidine-1-carboxylate (50 mg, 0.102 mmol, 1 equiv) as the starting material to give the crude product 7-(piperidin-4-yl)-5-((3-(trifluoromethyl)pyridin-2-yl)methyl)pyrido[3,2-d]pyrimidin-6(5H)-one (40 mg) as a white solid. MS m/z: 390 [M+H]⁺.

Step 3: 7-(1-(2-fluoro-6-methylphenyl)piperidin-4-yl)-5-((3-(trifluoromethyl)pyridin-2-yl)methyl)pyrido[3,2-d]pyrimidin-6(5H)-one: Followed general procedure G using 7-(piperidin-4-yl)-5-((3-(trifluoromethyl)pyridin-2-yl)methyl)pyrido[3,2-d]pyrimidin-6(5H)-one (40 mg, 0.102 mmol, 1 equiv) and 2-bromo-1-fluoro-3-methylbenzene (23.2 mg, 0.123 mmol, 1.2 equiv) as the starting materials to give 7-(1-(2-fluoro-6-methylphenyl)piperidin-4-yl)-5-((3-(trifluoromethyl)pyridin-2-yl)methyl)pyrido[3,2-d]pyrimidin-6(5H)-one (17.3 mg, 33.8%) as a white solid. ¹H NMR (300 MHz, Chloroform-d) δ 9.29 (s, 1H), 9.25 (s, 1H), 8.82-8.77 (m, 1H), 8.14-8.06 (m, 2H), 7.49-7.39 (m, 1H), 7.04-6.95 (m, 2H), 6.95-6.84 (m, 1H), 5.91 (s, 2H), 3.36-3.14 (m, 3H), 3.14-3.04 (m, 2H), 2.36 (s, 3H), 2.12-2.04 (m, 2H), 1.96-1.80 (m, 2H). MS m/z: 498.10 [M+H]⁺.

### 7-(1-(2-Fluoro-4-methoxypyridin-3-yl)piperidin-4-yl)-3-methyl-5-((3-(trifluoromethyl)pyridin-2-yl)methyl)pyrido[2,3-b]pyrazin-6(5H)-one (85)

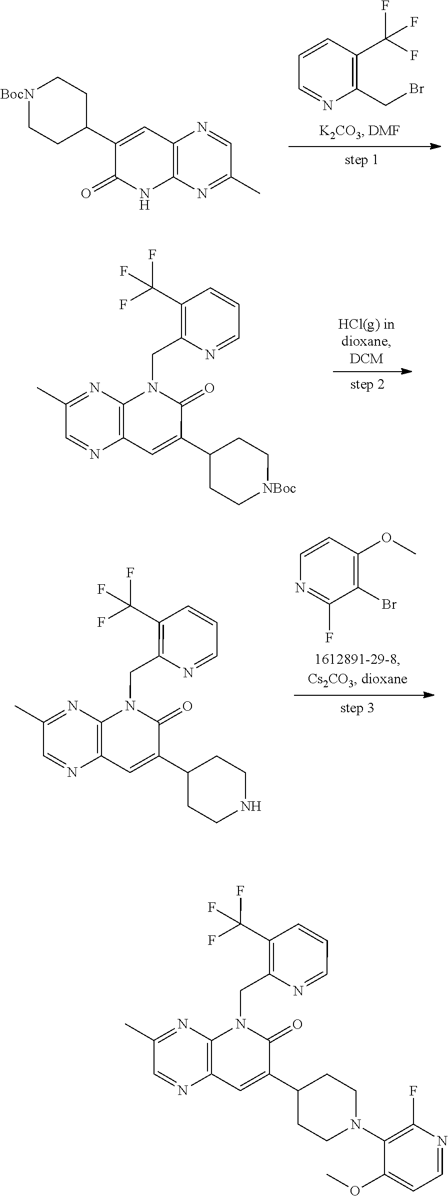

Step 1: tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (90 mg, 0.261 mmol, 1 equiv) and 2-(bromomethyl)-3-(trifluoromethyl)pyridine (62.7 mg, 0.261 mmol, 1.5 equiv) as the starting materials to give tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (55 mg, 48%) as an off-white solid. MS m/z: 504 [M+H]⁺.

Step 2: 3-methyl-7-(piperidin-4-yl)-5-((3-(trifluoromethyl)pyridin-2-yl)methyl)pyrido[2,3-b]pyrazin-6(5H)-one: Followed general procedure F using tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (55 mg, 0.109 mmol, 1 equiv) as the starting material to give the crude product 3-methyl-7-(piperidin-4-yl)-5-((3-(trifluoromethyl)pyridin-2-yl)methyl)pyrido[2,3-b]pyrazin-6(5H)-one (35 mg) as an off-white solid. MS m/z: 404 [M+H]⁺.

Step 3: 7-(1-(2-fluoro-4-methoxypyridin-3-yl)piperidin-4-yl)-3-methyl-5-((3-(trifluoromethyl)pyridin-2-yl)methyl)pyrido[2,3-b]pyrazin-6(5H)-one: Followed general procedure G using 3-methyl-7-(piperidin-4-yl)-5-((3-(trifluoromethyl)pyridin-2-yl)methyl)pyrido[2,3-b]pyrazin-6(5H)-one (35 mg, 0.087 mmol, 1 equiv) and 3-bromo-2-fluoro-4-methoxypyridine (21.4 mg, 0.104 mmol, 1.2 equiv) as the starting materials to give 7-(1-(2-fluoro-4-methoxypyridin-3-yl)piperidin-4-yl)-3-methyl-5-((3-(trifluoromethyl)pyridin-2-yl)methyl)pyrido[2,3-b]pyrazin-6(5H)-one (5.4 mg, 11.5%) as a brown yellow solid. ¹H NMR (400 MHz, Methanol-d₄) δ 8.45-8.40 (m, 2H), 8.15 (dd, 1H), 7.90 (d, 1H), 7.79 (d, 1H), 7.44-7.37 (m, 1H), 6.97 (d, 1H), 6.03 (s, 2H), 3.97 (s, 3H), 3.28-3.19 (m, 4H), 3.11-3.00 (m, 1H), 2.51 (s, 3H), 2.03-1.94 (m, 2H), 1.92-1.78 (m, 2H). MS m/z: 529.15 [M+H]⁺.

### 5-((3-Fluoropyridin-2-yl)methyl)-8-methyl-7-(1-(4-(trifluoromethyl)pyridin-3-yl)piperidin-4-yl)pyrido[2,3-b]pyrazin-6(5H)-one (86)

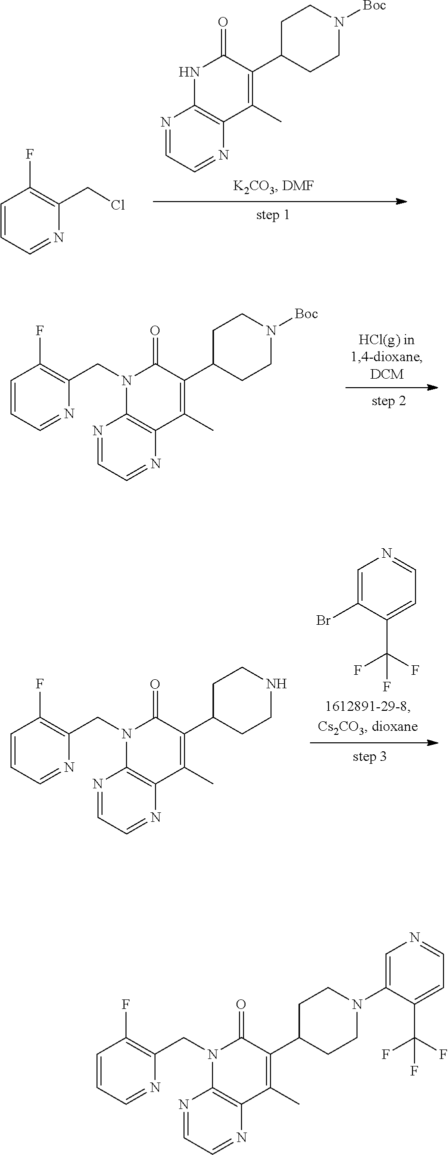 [SMILES: Fc1cccnc1CCl ; Cc1c(C2CCN(C(=O)OC(C)(C)C)CC2)c(=O)[nH]c2nccnc12 ; Cc1c(C2CCN(C(=O)OC(C)(C)C)CC2)c(=O)n(Cc2ncccc2F)c2nccnc12 ; Cc1c(C2CCNCC2)c(=O)n(Cc2ncccc2F)c2nccnc12 ; FC(F)(F)c1ccncc1Br ; Cc1c(C2CCN(c3cnccc3C(F)(F)F)CC2)c(=O)n(Cc2ncccc2F)c2nccnc12]

Step 1: tert-butyl 4-(5-((3-fluoropyridin-2-yl)methyl)-8-methyl-6-oxo-5,6-dihydropyrido[2,3-b]pyrazin-7-yl)piperidine-1-carboxylate: Followed general procedure E using 2-(chloromethyl)-3-fluoropyridine (100 mg, 0.687 mmol, 1 equiv) and tert-butyl 4-(8-methyl-6-oxo-5,6-dihydropyrido[2,3-b]pyrazin-7-yl)piperidine-1-carboxylate (236 mg, 0.687 mmol, 1.5 equiv) as the starting materials to give tert-butyl 4-(5-((3-fluoropyridin-2-yl)methyl)-8-methyl-6-oxo-5,6-dihydropyrido[2,3-b]pyrazin-7-yl)piperidine-1-carboxylate (100 mg, 32%) as a white solid. MS m/z: 454 [M+H]⁺.

Step 2: 5-((3-fluoropyridin-2-yl)methyl)-8-methyl-7-(piperidin-4-yl)pyrido[2,3-b]pyrazin-6(5H)-one: Followed general procedure F using tert-butyl 4-(5-((3-fluoropyridin-2-yl)methyl)-8-methyl-6-oxo-5,6-dihydropyrido[2,3-b]pyrazin-7-yl)piperidine-1-carboxylate (100 mg, 0.220 mmol, 1 equiv) as the starting material to give the crude product 5-((3-fluoropyridin-2-yl)methyl)-8-methyl-7-(piperidin-4-yl)pyrido[2,3-b]pyrazin-6(5H)-one (75 mg) as a white solid. MS m/z: 354 [M+H]⁺.

Step 3: 5-((3-fluoropyridin-2-yl)methyl)-8-methyl-7-(1-(4-(trifluoromethyl)pyridin-3-yl)piperidin-4-yl)pyrido[2,3-b]pyrazin-6(5H)-one: Followed general procedure G using 5-((3-fluoropyridin-2-yl)methyl)-8-methyl-7-(piperidin-4-yl)pyrido[2,3-b]pyrazin-6(5H)-one (75 mg, 0.212 mmol, 1 equiv) and 3-bromo-4-(trifluoromethyl)pyridine (57.6 mg, 0.254 mmol, 1.2 equiv) as the starting materials to 5-((3-fluoropyridin-2-yl)methyl)-8-methyl-7-(1-(4-(trifluoromethyl)pyridin-3-yl)piperidin-4-yl)pyrido[2,3-b]pyrazin-6(5H)-one (52.0 mg, 49.0%) as a white solid. ¹H NMR (400 MHz, Chloroform-d) δ 8.67 (s, 1H), 8.50-8.44 (m, 2H), 8.36 (d, 1H), 8.17-8.11 (m, 1H), 7.48 (d, 1H), 7.42-7.32 (m, 1H), 7.18-7.10 (m, 1H), 5.91 (d, 2H), 3.41 (d, 1H), 3.37-3.29 (m, 2H), 3.09-2.98 (m, 2H), 2.78-2.63 (m, 5H), 1.75-1.68 (m, 2H). MS m/z: 499.05 [M+H]⁺.

### 7-(1-(2-Fluoro-6-methylphenyl)piperidin-4-yl)-5-((3-(trifluoromethoxy)pyridin-2-yl)methyl)pyrido[2,3-b]pyrazin-6(5H)-one (87)

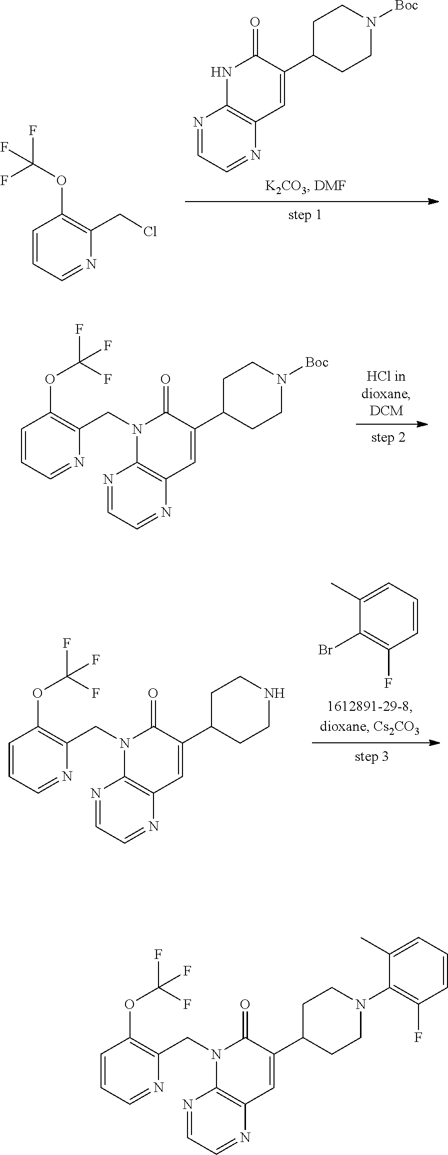

Step 1: tert-butyl 4-(6-oxo-5-((3-(trifluoromethoxy)pyridin-2-yl)methyl)-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (91 mg, 0.276 mmol, 1 equiv) and 2-(chloromethyl)-3-(trifluoromethoxy)pyridine (58.2 mg, 0.276 mmol, 1.5 equiv) as the starting materials to give tert-butyl 4-(6-oxo-5-((3-(trifluoromethoxy)pyridin-2-yl)methyl)-5,6-dihydropyrido[2,3-b]pyrazin-7-yl)piperidine-1-carboxylate (90 mg, 64%) as a yellow solid. MS m/z: 505 [M+H]⁺.

Step 2: 7-(piperidin-4-yl)-5-((3-(trifluoromethoxy)pyridin-2-yl)methyl)pyrido[2,3-b]pyrazin-6(5H)-one: Followed general procedure F using tert-butyl 4-(6-oxo-5-((3-(trifluoromethoxy)pyridin-2-yl)methyl)-5,6-dihydropyrido[2,3-b]pyrazin-7-yl)piperidine-1-carboxylate (90 mg, 0.178 mmol, 1 equiv) as the starting material to give the crude product 7-(piperidin-4-yl)-5-((3-(trifluoromethoxy)pyridin-2-yl)methyl)pyrido[2,3-b]pyrazin-6(5H)-one (60 mg, 83%) as an off-white solid. MS m/z: 405 [M+H]⁺.

Step 3: 7-(1-(2-fluoro-6-methylphenyl)piperidin-4-yl)-5-((3-(trifluoromethoxy)pyridin-2-yl)methyl)pyrido[2,3-b]pyrazin-6(5H)-one: Followed general procedure G using 7-(piperidin-4-yl)-5-((3-(trifluoromethoxy)pyridin-2-yl)methyl)pyrido[2,3-b]pyrazin-6(5H)-one (60 mg, 0.148 mmol, 1 equiv) and 3-bromo-5-fluoro-2-methylpyridine (33.5 mg, 0.178 mmol, 1.2 equiv) as the starting materials to give 7-(1-(2-fluoro-6-methylphenyl)piperidin-4-yl)-5-((3-(trifluoromethoxy)pyridin-2-yl)methyl)pyrido[2,3-b]pyrazin-6(5H)-one (49.8 mg, 60.3%) as a light yellow solid. ¹H NMR (300 MHz, Chloroform-d) δ 8.47 (d, 1H), 8.37 (d, 1H), 8.25 (dd, 1H), 7.89 (d, 1H), 7.59 (dt, 1H), 7.24-7.17 (m, 1H), 7.04-6.94 (m, 2H), 6.92-6.83 (m, 1H), 5.95 (s, 2H), 3.36 (t, 2H), 3.26-3.08 (m, 3H), 2.39 (s, 3H), 2.10-2.00 (m, 2H), 1.98-1.81 (m, 2H). MS m/z: 514.15 [M+H]⁺.

### 7-(4-(2-Fluoro-6-methylphenyl)piperazin-1-yl)-8-methyl-5-((3-(trifluoromethyl)pyridin-2-yl)methyl)pyrido[2,3-b]pyrazin-6(5H)-one (88)

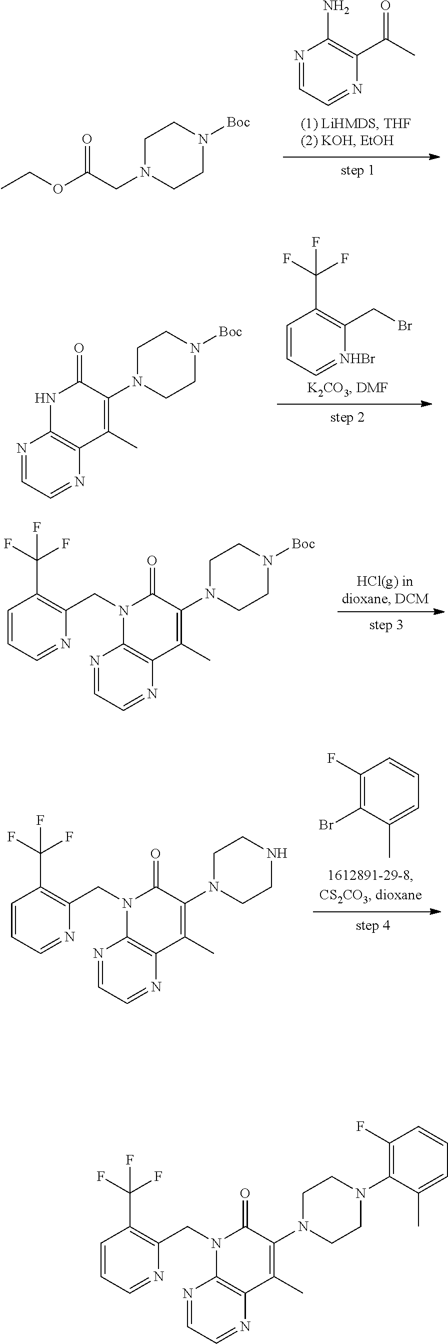

Step 1: tert-butyl 4-(8-methyl-6-oxo-5,6-dihydropyrido[2,3-b]pyrazin-7-yl)piperazine-1-carboxylate: Followed general procedure D using tert-butyl 4-(2-ethoxy-2-oxoethyl)piperazine-1-carboxylate (200 mg, 0.735 mmol, 1 equiv) and 1-(3-aminopyrazin-2-yl)ethan-1-one (121 mg, 0.882 mmol, 1.2 equiv) as the starting materials to give tert-butyl 4-(8-methyl-6-oxo-5,6-dihydropyrido[2,3-b]pyrazin-7-yl)piperazine-1-carboxylate (70 mg, 27%) as a yellow oil. MS m/z: 346 [M+H]⁺.

Step 2: tert-butyl 4-(8-methyl-6-oxo-5-((3-(trifluoromethyl)pyridin-2-yl)methyl)-5,6-dihydropyrido[2,3-b]pyrazin-7-yl)piperazine-1-carboxylate: Followed general procedure E using tert-butyl 4-(3-methoxy-6-oxo-5-(2-(trifluoromethyl)benzyl)-5,6-dihydropyrido[2,3-b]pyrazin-7-yl)piperidine-1-carboxylate (70 mg, 0.202 mmol, 1 equiv) and 2-(bromomethyl)-3-(trifluoromethyl)pyridine (65.1 mg, 0.202 mmol, 1 equiv) as the starting materials to give tert-butyl 4-(8-methyl-6-oxo-5-((3-(trifluoromethyl)pyridin-2-yl)methyl)-5,6-dihydropyrido[2,3-b]pyrazin-7-yl)piperazine-1-carboxylate (60 mg, 58%) as a yellow oil. MS m/z: 505 [M+H]⁺.

Step 3: 8-methyl-7-(piperazin-1-yl)-5-((3-(trifluoromethyl)pyridin-2-yl)methyl)pyrido[2,3-b]pyrazin-6(5H)-one: Followed general procedure F using tert-butyl 4-(8-methyl-6-oxo-5-((3-(trifluoromethyl)pyridin-2-yl)methyl)-5,6-dihydropyrido[2,3-b]pyrazin-7-yl)piperazine-1-carboxylate (60 mg, 0.119 mmol, 1 equiv) as the starting material to give the crude product 8-methyl-7-(piperazin-1-yl)-5-((3-(trifluoromethyl)pyridin-2-yl)methyl)pyrido[2,3-b]pyrazin-6(5H)-one (58 mg) as a yellow solid. MS m/z: 405 [M+H]⁺.

Step 4: 7-(4-(2-fluoro-6-methylphenyl)piperazin-1-yl)-8-methyl-5-((3-(trifluoromethyl)pyridin-2-yl)methyl)pyrido[2,3-b]pyrazin-6(5H)-one: Followed general procedure G using 8-methyl-7-(piperazin-1-yl)-5-((3-(trifluoromethyl)pyridin-2-yl)methyl)pyrido[2,3-b]pyrazin-6(5H)-one (58 mg, 0.143 mmol, 1 equiv) and 2-bromo-1-fluoro-3-methylbenzene (25.2 mg, 0.134 mmol, 1.5 equiv) as the starting materials to give 7-(4-(2-fluoro-6-methylphenyl)piperazin-1-yl)-8-methyl-5-((3-(trifluoromethyl)pyridin-2-yl)methyl)pyrido[2,3-b]pyrazin-6(5H)-one (23.4 mg, 31.6%) as a yellow solid. ¹H NMR (400 MHz, Chloroform-d) δ 8.46 (d, 1H), 8.42 (d, 1H), 8.26 (d, 1H), 7.96 (dd, 1H), 7.02-6.95 (m, 2H), 6.91-6.84 (m, 1H), 6.02 (s, 2H), 3.65-2.93 (m, 8H), 2.71 (s, 3H), 2.35 (s, 3H), 2.05-1.99 (m, 1H). MS m/z: 513.10 [M+H]⁺.

### 7-(4-(2-Fluoro-6-methylphenyl)piperidin-1-yl)-5-((3-(trifluoromethyl)pyrazin-2-yl)methyl) pyrido[2,3-b]pyrazin-6(5H)-one (89)

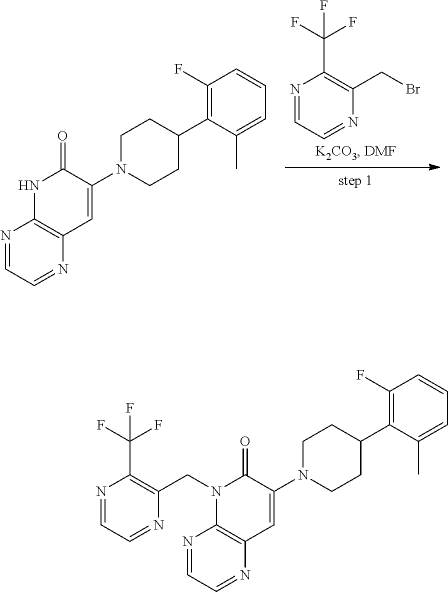

Step 1: 7-(4-(2-fluoro-6-methylphenyl)piperidin-1-yl)-5-((3-(trifluoromethyl)pyrazin-2-yl)methyl) pyrido[2,3-b]pyrazin-6(5H)-one: Followed general procedure E using 7-(4-(2-fluoro-6-methylphenyl)piperidin-1-yl)pyrido[2,3-b]pyrazin-6(5H)-one (35 mg, 0.103 mmol, 1 equiv) and 2-(bromomethyl)-3-(trifluoromethyl)pyrazine (30 mg, 0.124 mmol, 1.2 equiv) as the starting materials to give 7-(4-(2-fluoro-6-methylphenyl)piperidin-1-yl)-5-((3-(trifluoromethyl)pyrazin-2-yl)methyl)pyrido[2,3-b]pyrazin-6(5H)-one (28.5 mg, 53.0%) as a yellow solid. ¹H NMR (400 MHz, Chloroform-d) δ 8.51 (d, 1H), 8.45 (d, 1H), 8.34 (d, 1H), 8.16 (d, 1H), 7.30 (s, 1H), 7.09-7.04 (m, 1H), 6.94 (d, 1H), 6.88-6.83 (m, 1H), 6.09 (s, 2H), 4.23 (d, 2H), 3.09-3.01 (m, 1H), 2.92 (t, 2H), 2.49-2.40 (s, 5H), 1.80 (d, 2H). MS m/z: 499.15 [M+H]⁺.

### 3-(1-(2-Fluoro-6-methylphenyl)piperidin-4-yl)-7-methyl-1-((3-(trifluoromethyl)pyridin-2-yl)methyl)-1,5-naphthyridin-2(1H)-one (90)

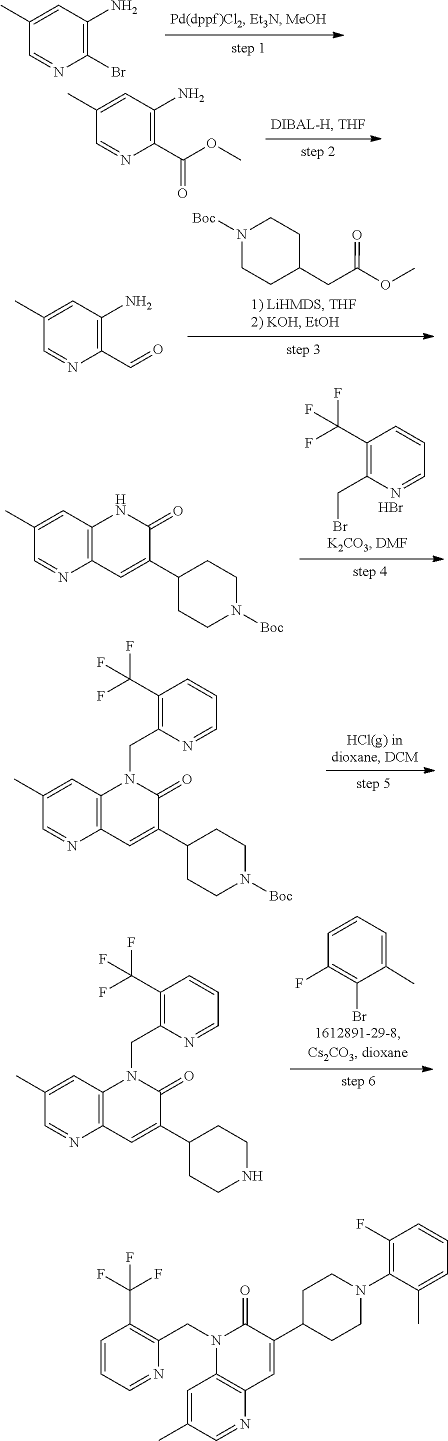

Step 1: methyl 3-amino-5-methylpicolinate: Followed general procedure H using 2-bromo-5-methylpyridin-3-amine (1.5 g, 8.02 mmol, 1 equiv) as the starting material to give methyl 3-amino-5-methylpicolinate (410 mg, 30%) as a yellow solid. MS m/z: 167 [M+H]⁺.

Step 2: 3-amino-5-methylpicolinaldehyde: Followed general procedure B using methyl 3-amino-5-methylpicolinate (410 mg, 2.48 mmol, 1 equiv) and DIBAL-H (526 mg, 3.7 mmol, 1.5 equiv) as the starting materials to give 3-amino-5-methylpicolinaldehyde (260 mg, 77%) as a yellow solid. MS m/z: 137 [M+H]⁺.

Step 3: tert-butyl 4-(7-methyl-2-oxo-1,2-dihydro-1,5-naphthyridin-3-yl)piperidine-1-carboxylate: Followed general procedure D using 3-amino-5-methylpicolinaldehyde (260 mg, 1.91 mmol, 1 equiv) and tert-butyl 4-(2-methoxy-2-oxoethyl)piperidine-1-carboxylate (737 mg, 2.87 mmol, 1.5 equiv) as the starting materials to give tert-butyl 4-(7-methyl-2-oxo-1,2-dihydro-1,5-naphthyridin-3-yl)piperidine-1-carboxylate (180 mg, 27%) as a yellow solid. MS m/z: 344 [M+H]⁺.

Step 4: tert-butyl 4-(7-methyl-2-oxo-1-((3-(trifluoromethyl)pyridin-2-yl)methyl)-1,2-dihydro-1,5-naphthyridin-3-yl)piperidine-1-carboxylate: Followed general procedure E using tert-butyl 4-(7-methyl-2-oxo-1,2-dihydro-1,5-naphthyridin-3-yl)piperidine-1-carboxylate (180 mg, 0.524 mmol, 1 equiv) and 2-(bromomethyl)-3-(trifluoromethyl)pyridine hydrobromide (113 mg, 0.472 mmol, 0.9 equiv) as the starting materials to give the product tert-butyl 4-(7-methyl-2-oxo-1-((3-(trifluoromethyl)pyridin-2-yl)methyl)-1,2-dihydro-1,5-naphthyridin-3-yl)piperidine-1-carboxylate (95 mg, 36%) as an off-white oil. MS m/z: 503 [M+H]⁺.

Step 5: 7-methyl-3-(piperidin-4-yl)-1-((3-(trifluoromethyl)pyridin-2-yl)methyl)-1,5-naphthyridin-2(1H)-one: Followed general procedure F using tert-butyl 4-(7-methyl-2-oxo-1-((3-(trifluoromethyl)pyridin-2-yl)methyl)-1,2-dihydro-1,5-naphthyridin-3-yl)piperidine-1-carboxylate (40 mg, 0.08 mmol, 1 equiv) as the starting material to give the crude product 7-methyl-3-(piperidin-4-yl)-1-((3-(trifluoromethyl)pyridin-2-yl)methyl)-1,5-naphthyridin-2(1H)-one (20 mg) as a yellow oil. MS m/z: 403 [M+H]⁺.

Step 6: 3-(1-(2-fluoro-6-methylphenyl)piperidin-4-yl)-7-methyl-1-((3-(trifluoromethyl) pyridin-2-yl)methyl)-1,5-naphthyridin-2(1H)-one: Followed general procedure G using 7-methyl-3-(piperidin-4-yl)-1-((3-(trifluoromethyl)pyridin-2-yl)methyl)-1,5-naphthyridin-2(1H)-one (20 mg, 0.05 mmol, 1 equiv) and 2-bromo-1-fluoro-3-methylbenzene (10.3 mg, 0.055 mmol, 1.1 equiv) as the starting materials to give 3-(1-(2-fluoro-6-methylphenyl)piperidin-4-yl)-7-methyl-1-((3-(trifluoromethyl)pyridin-2-yl)methyl)-1,5-naphthyridin-2(1H)-one (6.9 mg, 26.8%) as a white solid. ¹H NMR (300 MHz, Chloroform-d) δ 8.78 (d, J=5.0 Hz, 1H), 8.61 (d, J=2.1 Hz, 1H), 8.14 (s, 1H), 8.05 (dd, J=7.9, 1.7 Hz, 1H), 7.92-7.85 (m, 1H), 7.46-7.37 (m, 1H), 7.00-6.91 (m, 2H), 6.89-6.79 (m, 1H), 5.84 (s, 2H), 3.31-3.19 (m, 2H), 3.18-3.00 (m, 3H), 2.52 (s, 3H), 2.32 (s, 3H), 2.03 (d, J=11.8 Hz, 2H), 1.94-1.78 (m, 2H). MS m/z: 511.15 [M+H]⁺.

### 7-(4-(2-fluoro-6-methylphenyl)piperidin-1-yl)-5-((3-(trifluoromethyl)pyridin-2-yl)methyl) pyrido[2,3-b]pyrazin-6(5H)-one (91)

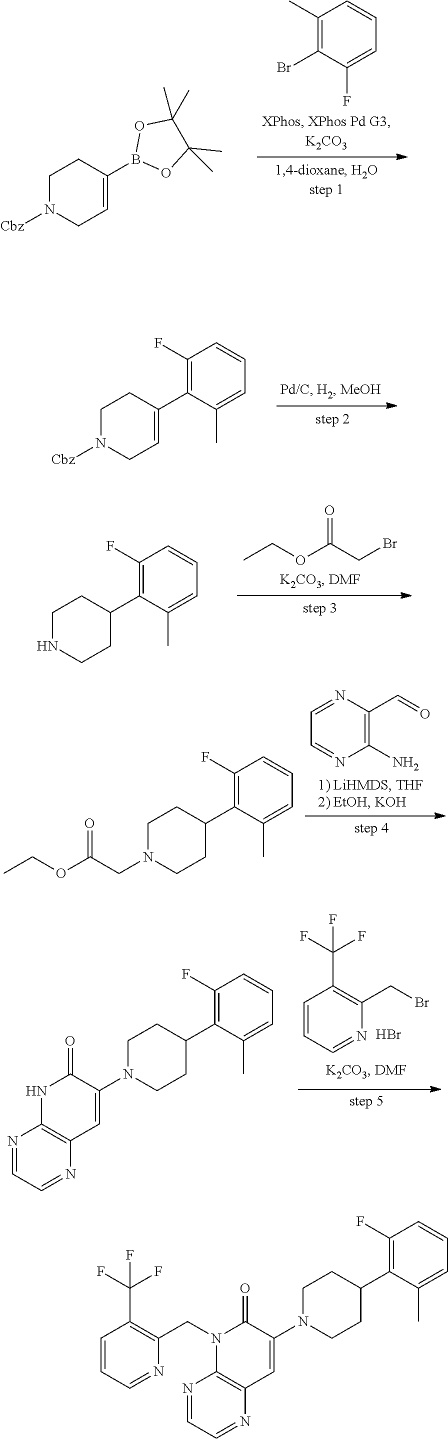

Step 1: benzyl 4-(2-fluoro-6-methylphenyl)-3,6-dihydropyridine-1(2H)-carboxylate: Followed general procedure Y using benzyl 4-(4,4,5,5-tetramethyl-1,3,2-dioxaborolan-2-yl)-3,6-dihydropyridine-1(2H)-carboxylate (2 g, 5.82 mmol, 1 equiv) and 2-bromo-1-fluoro-3-methylbenzene (1.32 g, 6.99 mmol, 1.2 equiv) as the starting materials to give benzyl 4-(2-fluoro-6-methylphenyl)-3,6-dihydropyridine-1(2H)-carboxylate (1.6 g, 84%) as a colorless oil. MS m/z: 326 [M+H]⁺.

Step 2: 4-(2-fluoro-6-methylphenyl)piperidine: Followed general procedure K using benzyl 4-(2-fluoro-6-methylphenyl)-3,6-dihydropyridine-1(2H)-carboxylate (1 g, 3.07 mmol, 1 equiv) as the starting material to give the crude product 4-(2-fluoro-6-methylphenyl)piperidine (550 mg) as a light yellow oil. MS m/z: 194 [M+H]⁺.

Step 3: ethyl 2-(4-(2-fluoro-6-methylphenyl)piperidin-1-yl)acetate: Followed general procedure E using 4-(2-fluoro-6-methylphenyl)piperidine (550 mg, 2.84 mmol, 1 equiv) and ethyl 2-bromoacetate (570 mg, 3.41 mmol, 1.2 equiv) as the starting materials to give ethyl 2-(4-(2-fluoro-6-methylphenyl)piperidin-1-yl)acetate (400 mg, 50%) as a light yellow oil. MS m/z: 280 [M+H]⁺.

Step 4: 7-(4-(2-fluoro-6-methylphenyl)piperidin-1-yl)pyrido[2,3-b]pyrazin-6(5H)-one: Followed general procedure D using 3-aminopyrazine-2-carbaldehyde (105 mg, 0.859 mmol, 1.2 equiv) and ethyl 2-(4-(2-fluoro-6-methylphenyl)piperidin-1-yl)acetate (200 mg, 0.716 mmol, 1 equiv) as the starting materials to give 7-(4-(2-fluoro-6-methylphenyl)piperidin-1-yl)pyrido[2,3-b]pyrazin-6(5H)-one (60 mg, 24%) as a yellow solid. MS m/z: 339 [M+H]⁺.

Step 5: 7-(4-(2-fluoro-6-methylphenyl)piperidin-1-yl)-5-((3-(trifluoromethyl)pyridin-2-yl)methyl) pyrido[2,3-b]pyrazin-6(5H)-one: Followed general procedure E using 7-(4-(2-fluoro-6-methylphenyl)piperidin-1-yl)pyrido[2,3-b]pyrazin-6(5H)-one (60 mg, 0.177 mmol, 1 equiv) and 2-(bromomethyl)-3-(trifluoromethyl)pyridine hydrobromide (62.6 mg, 0.195 mmol, 1.1 equiv) as the starting materials to give 7-(4-(2-fluoro-6-methylphenyl)piperidin-1-yl)-5-((3-(trifluoromethyl)pyridin-2-yl)methyl)pyrido[2,3-b]pyrazin-6(5H)-one (30.1 mg, 34%) as a yellow solid. ¹H NMR (400 MHz, Chloroform-d) δ 8.43-8.41 (m, 1H), 8.31 (d, J=2.6 Hz, 1H), 8.18 (d, J=2.6 Hz, 1H), 7.98-7.95 (m, 1H), 7.31 (s, 1H), 7.24-7.22 (m, 1H), 7.09-7.04 (m, 1H), 6.94 (d, J=7.6 Hz, 1H), 6.88-6.83 (m, 1H), 6.04 (s, 2H), 4.26 (d, J=12.2 Hz, 2H), 3.09-3.01 (m, 1H), 2.92 (t, J=12.4 Hz, 2H), 2.40 (s, 5H), 1.79 (d, J=13.2 Hz, 2H). MS m/z: 498.15 [M+H]⁺.

### 7-(4-(2-Fluoro-6-methylphenyl)piperazin-1-yl)-3-methyl-5-((3-(trifluoromethyl)pyrazin-2-yl)methyl)pyrido[2,3-b]pyrazin-6(5H)-one (92)

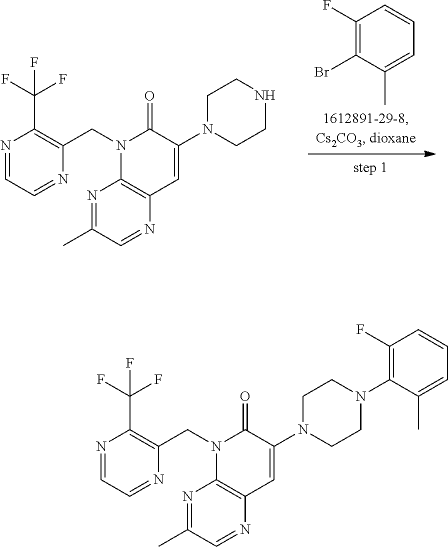

Step 1: 7-(4-(2-fluoro-6-methylphenyl)piperazin-1-yl)-3-methyl-5-((3-(trifluoromethyl)pyrazin-2-yl)methyl)pyrido[2,3-b]pyrazin-6(5H)-one: Followed general procedure G using 3-methyl-7-(piperazin-1-yl)-5-((3-(trifluoromethyl)pyrazin-2-yl)methyl)pyrido[2,3-b]pyrazin-6(5H)-one (50 mg, 0.123 mmol, 1 equiv) and 2-bromo-1-fluoro-3-methylbenzene (35 mg, 0.184 mmol, 1.5 equiv) as the starting materials to give 7-(4-(2-fluoro-6-methylphenyl)piperazin-1-yl)-3-methyl-5-((3-(trifluoromethyl)pyrazin-2-yl)methyl)pyrido[2,3-b]pyrazin-6(5H)-one (22.8 mg, 35.3%) as an orange solid. ¹H NMR (400 MHz, Chloroform-d) δ 8.52-8.44 (m, 2H), 8.26 (s, 1H), 7.25 (s, 1H), 7.04-6.95 (m, 2H), 6.90-6.84 (m, 1H), 6.09 (s, 2H), 3.95-3.06 (m, 8H), 2.48 (s, 3H), 2.37 (s, 3H). MS m/z: 514.15 [M+H]⁺.

### 7-(1-(2-Fluoro-6-methylphenyl)piperidin-4-yl)-4-methoxy-5-((3-(trifluoromethyl)pyrazin-2-yl)methyl)pyrido[3,2-d]pyrimidin-6(5H)-one (93)

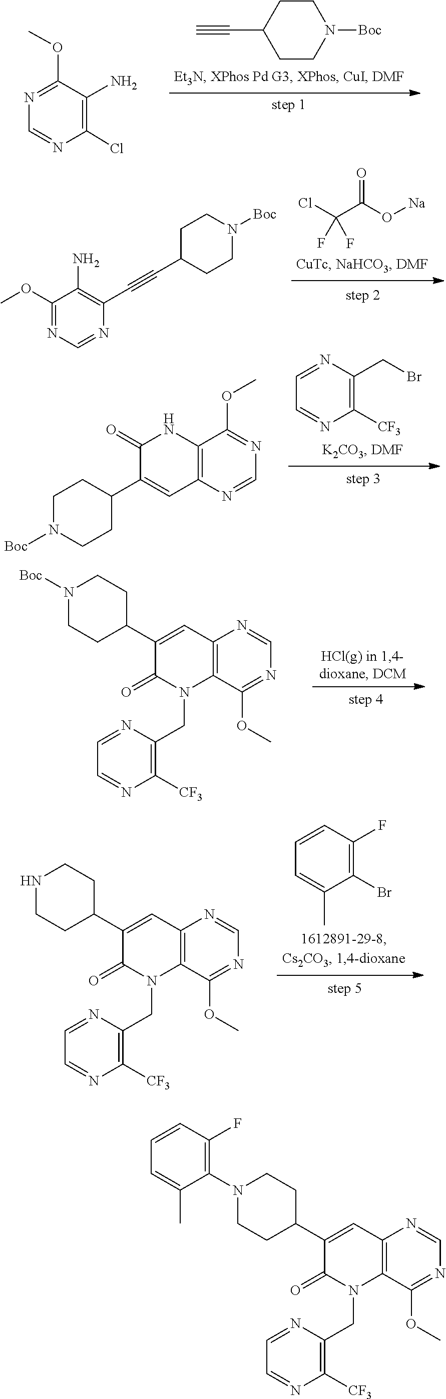

Step 1: tert-butyl 4-((5-amino-6-methoxypyrimidin-4-yl)ethynyl)piperidine-1-carboxylate: Followed general procedure Z using 4-chloro-6-methoxypyrimidin-5-amine (200 mg, 1.253 mmol, 1 equiv) and tert-butyl 4-ethynylpiperidine-1-carboxylate (314 mg, 1.504 mmol, 1.2 equiv) as the starting materials, XPhos (59.7 mg, 0.125 mmol, 0.1 equiv) and XPhos Pd G3 (106 mg, 0.125 mmol, 0.1 equiv) as the catalyst, DMF (1.5 mL) as the solvent to give tert-butyl 4-((5-amino-6-methoxypyrimidin-4-yl)ethynyl)piperidine-1-carboxylate (200 mg, 48%) as a yellow solid. MS m/z: 333 [M+H]⁺.

Step 2: tert-butyl 4-(4-methoxy-6-oxo-5,6-dihydropyrido[3,2-d]pyrimidin-7-yl)piperidine-1-carboxylate: Followed general procedure AA using tert-butyl 4-((5-amino-6-methoxypyrimidin-4-yl)ethynyl)piperidine-1-carboxylate (200 mg, 0.602 mmol, 1 equiv) and sodium 2-chloro-2,2-difluoroacetate (275 mg, 1.806 mmol, 3 equiv) as the starting materials to give tert-butyl 4-(4-methoxy-6-oxo-5,6-dihydropyrido[3,2-d]pyrimidin-7-yl)piperidine-1-carboxylate (70 mg, 32%) as a light yellow solid. MS m/z: 361 [M+H]⁺.

Step 3: tert-butyl 4-(4-methoxy-6-oxo-5-((3-(trifluoromethyl)pyrazin-2-yl)methyl)-5,6-dihydropyrido[3,2-d]pyrimidin-7-yl)piperidine-1-carboxylate: Followed general procedure E using tert-butyl 4-(4-methoxy-6-oxo-5,6-dihydropyrido[3,2-d]pyrimidin-7-yl)piperidine-1-carboxylate (70 mg, 0.194 mmol, 1 equiv) and 2-(bromomethyl)-3-(trifluoromethyl)pyrazine (70 mg, 0.291 mmol, 1.5 equiv) as the starting materials to give tert-butyl 4-(4-methoxy-6-oxo-5-((3-(trifluoromethyl)pyrazin-2-yl)methyl)-5,6-dihydropyrido[3,2-d]pyrimidin-7-yl)piperidine-1-carboxylate (60 mg, 59%) as a light yellow solid. MS m/z: 521 [M+H]⁺.

Step 4: 4-methoxy-7-(piperidin-4-yl)-5-((3-(trifluoromethyl)pyrazin-2-yl)methyl)pyrido[3,2-d]pyrimidin-6(5H)-one: Followed general procedure F using tert-butyl 4-(4-methoxy-6-oxo-5-((3-(trifluoromethyl)pyrazin-2-yl)methyl)-5,6-dihydropyrido[3,2-d]pyrimidin-7-yl)piperidine-1-carboxylate (60 mg, 0.115 mmol, 1 equiv) as the starting material to give the crude product 4-methoxy-7-(piperidin-4-yl)-5-((3-(trifluoromethyl)pyrazin-2-yl)methyl)pyrido[3,2-d]pyrimidin-6(5H)-one (35 mg) as an off-white solid. MS m/z: 421 [M+H]⁺.

Step 5: 7-(1-(2-fluoro-6-methylphenyl)piperidin-4-yl)-4-methoxy-5-((3-(trifluoromethyl)pyrazin-2-yl)methyl)pyrido[3,2-d]pyrimidin-6(5H)-one: Followed general procedure G using 4-methoxy-7-(piperidin-4-yl)-5-((3-(trifluoromethyl)pyrazin-2-yl)methyl)pyrido[3,2-d]pyrimidin-6(5H)-one (35 mg, 0.083 mmol, 1 equiv) and 2-bromo-1-fluoro-3-methylbenzene (23.6 mg, 0.125 mmol, 1.5 equiv) as the starting materials to give 7-(1-(2-fluoro-6-methylphenyl)piperidin-4-yl)-4-methoxy-5-((3-(trifluoromethyl)pyrazin-2-yl)methyl)pyrido[3,2-d]pyrimidin-6(5H)-one (9.6 mg, 24.9%) as an off-white solid. ¹H NMR (300 MHz, Methanol-d₄) δ 8.80 (d, J=2.4 Hz, 1H), 8.72-8.65 (m, 2H), 8.04 (s, 1H), 7.03-6.95 (m, 2H), 6.91-6.81 (m, 1H), 5.93 (s, 2H), 4.14 (s, 3H), 3.29-3.22 (m, 2H), 3.18-3.05 (m, 3H), 2.34 (s, 3H), 2.14-2.04 (m, 2H), 1.98-1.89 (m, 2H). MS m/z: 529.15 [M+H]⁺.

### 3-(1-(2-Fluoro-6-methylphenyl)piperidin-4-yl)-7-methyl-1-((3-(trifluoromethyl)pyridin-2-yl)methyl)-1,8-naphthyridin-2(1H)-one (94)

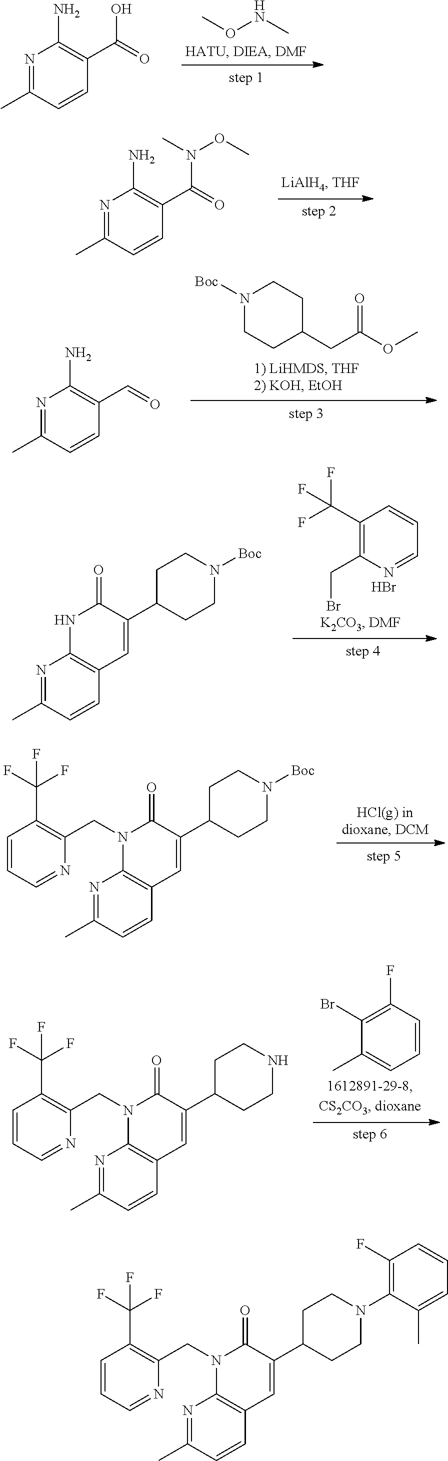

Step 1: 2-amino-N-methoxy-N,6-dimethylnicotinamide: Followed general procedure A using 2-amino-6-methylnicotinic acid (600 mg, 3.94 mmol, 1 equiv) and N,O-dimethylhydroxylamine (361 mg, 5.91 mmol, 1.5 equiv) as the starting materials to give 2-amino-N-methoxy-N,6-dimethylnicotinamide (530 mg, 68%) as a yellow solid. MS m/z: 196 [M+H]⁺.

Step 2: 2-amino-6-methylnicotinaldehyde: Followed general procedure B using 2-amino-N-methoxy-N,6-dimethylnicotinamide (380 mg, 1.94 mmol, 1 equiv) as the starting material to give 2-amino-6-methylnicotinaldehyde (240 mg, 90%) as a yellow oil. MS m/z: 137 [M+H]⁺.

Step 3: tert-butyl 4-(7-methyl-2-oxo-1,2-dihydro-1,8-naphthyridin-3-yl)piperidine-1-carboxylate: Followed general procedure D using 2-amino-6-methylnicotinaldehyde (240 mg, 1.76 mmol, 1 equiv) and tert-butyl 4-(2-methoxy-2-oxoethyl)piperidine-1-carboxylate (680 mg, 2.64 mmol, 1 equiv) as the starting materials to give tert-butyl 4-(7-methyl-2-oxo-1,2-dihydro-1,8-naphthyridin-3-yl)piperidine-1-carboxylate (90 mg, 14%) as a yellow oil. MS m/z: 344 [M+H]⁺.

Step 4: tert-butyl 4-(7-methyl-2-oxo-1-((3-(trifluoromethyl)pyridin-2-yl)methyl)-1,2-dihydro-1,8-naphthyridin-3-yl)piperidine-1-carboxylate: Followed general procedure E using tert-butyl 4-(7-methyl-2-oxo-1,2-dihydro-1,8-naphthyridin-3-yl)piperidine-1-carboxylate (90 mg, 0.262 mmol, 1 equiv) and 2-(bromomethyl)-3-(trifluoromethyl)pyridine hydrobromide (100 mg, 0.314 mmol, 1.2 equiv) as the starting materials to give tert-butyl 4-(7-methyl-2-oxo-1-((3-(trifluoromethyl)pyridin-2-yl)methyl)-1,2-dihydro-1,8-naphthyridin-3-yl)piperidine-1-carboxylate (50 mg, 37%) as a yellow oil. MS m/z: 503 [M+H]⁺.

Step 5: 7-methyl-3-(piperidin-4-yl)-1-((3-(trifluoromethyl)pyridin-2-yl)methyl)-1,8-naphthyridin-2(1H)-one: Followed general procedure F using tert-butyl 4-(7-methyl-2-oxo-1-((3-(trifluoromethyl)pyridin-2-yl)methyl)-1,2-dihydro-1,8-naphthyridin-3-yl)piperidine-1-carboxylate (50 mg, 0.099 mmol, 1 equiv) as the starting material to give 7-methyl-3-(piperidin-4-yl)-1-((3-(trifluoromethyl)pyridin-2-yl)methyl)-1,8-naphthyridin-2(1H)-one (30 mg, 75%) as a yellow oil. MS m/z: 403 [M+H]⁺.

Step 6: 7-methyl-3-(piperidin-4-yl)-1-((3-(trifluoromethyl)pyridin-2-yl)methyl)-1,8-naphthyridin-2(1H)-one: Followed general procedure G using 7-methyl-3-(piperidin-4-yl)-1-((3-(trifluoromethyl)pyridin-2-yl)methyl)-1,8-naphthyridin-2(1H)-one (30 mg, 0.074 mmol, 1 equiv) and 2-bromo-1-fluoro-3-methylbenzene (20.8 mg, 0.111 mmol, 1.5 equiv) as the starting materials to give 3-(1-(2-fluoro-6-methylphenyl)piperidin-4-yl)-7-methyl-1-((3-(trifluoromethyl)pyridin-2-yl)methyl)-1,8-naphthyridin-2(1H)-one (5 mg, 15.6%) as an off-white solid. ¹H NMR (400 MHz, Chloroform-d) δ 8.47-8.41 (m, 1H), 7.95 (d, J=7.9 Hz, 1H), 7.76 (d, J=7.8 Hz, 1H), 7.57 (s, 1H), 7.22-7.16 (m, 1H), 7.01-6.94 (m, 3H), 6.90-6.83 (m, 1H), 6.11 (s, 2H), 3.38-3.28 (m, 2H), 3.20-3.05 (m, 3H), 2.46 (s, 3H), 2.37 (s, 3H), 2.03 (d, J=12.3 Hz, 2H), 1.84-1.73 (m, 2H). MS m/z: 511.15 [M+H]⁺.

### 7-(4-(2-Fluoro-6-methylphenyl)piperidin-1-yl)-3-methyl-5-((3-(trifluoromethyl)pyrazin-2-yl)methyl)pyrido[2,3-b]pyrazin-6(5H)-one (95)

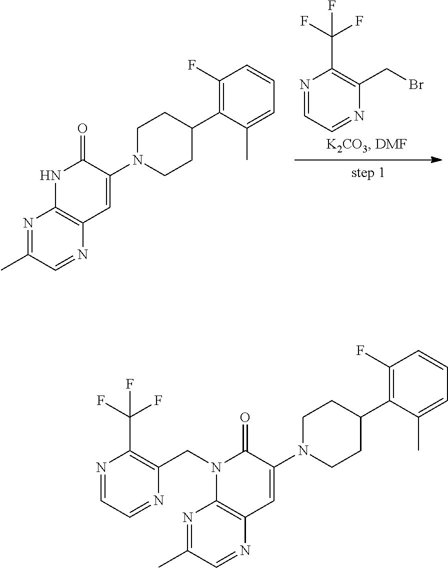

Step 1: 7-(4-(2-fluoro-6-methylphenyl)piperidin-1-yl)-3-methyl-5-((3-(trifluoromethyl)pyrazin-2-yl)methyl)pyrido[2,3-b]pyrazin-6(5H)-one: Followed general procedure E using 7-(4-(2-fluoro-6-methylphenyl)piperidin-1-yl)-3-methylpyrido[2,3-b]pyrazin-6(5H)-one (55 mg, 0.156 mmol, 1 equiv) and 2-(bromomethyl)-3-(trifluoromethyl)pyrazine (45.1 mg, 0.187 mmol, 1.2 equiv) as the starting materials to give 7-(4-(2-fluoro-6-methylphenyl)piperidin-1-yl)-3-methyl-5-((3-(trifluoromethyl)pyrazin-2-yl)methyl) pyrido[2,3-b]pyrazin-6(5H)-one (26.8 mg, 33.2%) as a yellow solid. ¹H NMR (400 MHz, Methanol-d₄) δ 8.57 (d, J=2.4 Hz, 1H), 8.53 (d, J=2.4 Hz, 1H), 8.31 (s, 1H), 7.26 (s, 1H), 7.10-7.05 (m, 1H), 6.97 (d, J=7.6 Hz, 1H), 6.88-6.82 (m, 1H), 6.09 (s, 2H), 4.06-4.02 (m, 2H), 3.11 (t, J=12.4 Hz, 1H), 2.87-2.80 (m, 2H), 2.43 (d, J=20.0 Hz, 8H), 1.79-1.74 (m, 2H). MS m/z: 513.15 [M+H]⁺.

### 7-(4-(2-Fluoro-6-methylphenyl)piperazin-1-yl)-5-((3-(trifluoromethyl)pyridin-2-yl)methyl)pyrido[2,3-b]pyrazin-6(5H)-one (96)

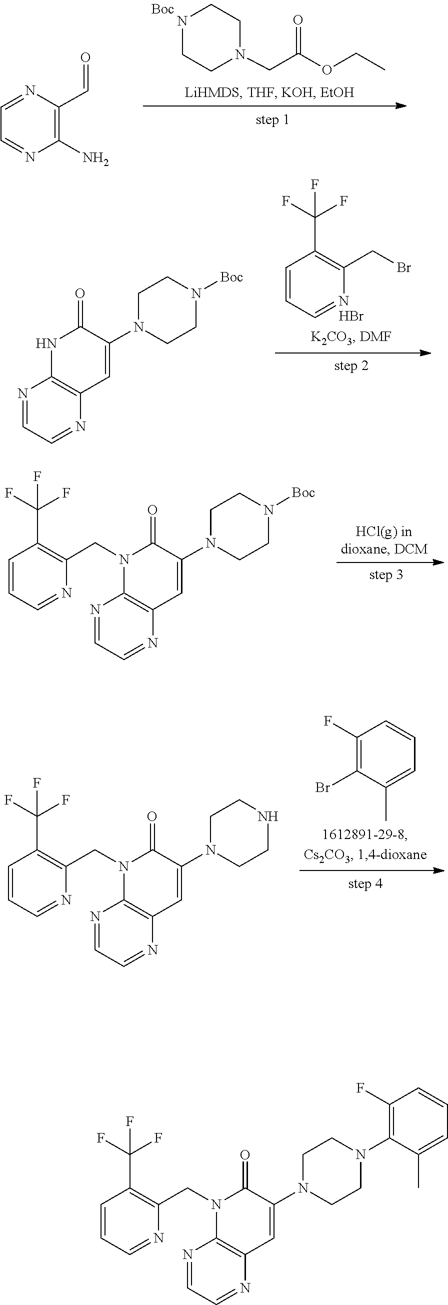

Step 1: tert-butyl 4-(6-oxo-5,6-dihydropyrido[2,3-b]pyrazin-7-yl)piperazine-1-carboxylate: Followed general procedure D using 3-aminopyrazine-2-carbaldehyde (180 mg, 1.47 mmol, 2 equiv) and tert-butyl 4-(2-ethoxy-2-oxoethyl)piperazine-1-carboxylate (200 mg, 0.73 mmol, 1 equiv) as the starting materials to give tert-butyl 4-(6-oxo-5,6-dihydropyrido[2,3-b]pyrazin-7-yl)piperazine-1-carboxylate (130 mg, 53%) as a yellow solid. MS m/z: 332 [M+H]⁺.

Step 2: tert-butyl 4-(6-oxo-5-((3-(trifluoromethyl)pyridin-2-yl)methyl)-5,6-dihydropyrido[2,3-b]pyrazin-7-yl)piperazine-1-carboxylate: Followed general procedure E using tert-butyl 4-(6-oxo-5,6-dihydropyrido[2,3-b]pyrazin-7-yl)piperazine-1-carboxylate (130 mg, 0.392 mmol, 1 equiv) and 2-(bromomethyl)-3-(trifluoromethyl)pyridine hydrogen bromide (138 mg, 0.431 mmol, 1.1 equiv) as the starting materials to give tert-butyl 4-(6-oxo-5-((3-(trifluoromethyl)pyridin-2-yl)methyl)-5,6-dihydropyrido[2,3-b]pyrazin-7-yl)piperazine-1-carboxylate (60 mg, 31%) as a yellow solid. MS m/z: 491 [M+H]⁺.

Step 3: 7-(piperazin-1-yl)-5-((3-(trifluoromethyl)pyridin-2-yl)methyl)pyrido[2,3-b]pyrazin-6(5H)-one: Followed general procedure F using tert-butyl 4-(6-oxo-5-((3-(trifluoromethyl)pyridin-2-yl)methyl)-5,6-dihydropyrido[2,3-b]pyrazin-7-yl)piperazine-1-carboxylate (60 mg, 0.12 mmol, 1 equiv) as the starting material to give the crude product 7-(piperazin-1-yl)-5-((3-(trifluoromethyl)pyridin-2-yl)methyl)pyrido[2,3-b]pyrazin-6(5H)-one (70 mg) as a white solid. MS m/z: 391 [M+H]⁺.

Step 4: 7-(4-(2-fluoro-6-methylphenyl)piperazin-1-yl)-5-((3-(trifluoromethyl)pyridin-2-yl)methyl)pyrido[2,3-b]pyrazin-6(5H)-one: Followed general procedure G using 7-(piperazin-1-yl)-5-((3-(trifluoromethyl)pyridin-2-yl)methyl)pyrido[2,3-b]pyrazin-6(5H)-one (70 mg, 0.179 mmol, 1 equiv) and 2-bromo-1-fluoro-3-methyl benzene (37 mg, 0.197 mmol, 1.1 equiv) as the starting materials to give 7-(4-(2-fluoro-6-methylphenyl)piperazin-1-yl)-5-((3-(trifluoromethyl)pyridin-2-yl)methyl)pyrido[2,3-b]pyrazin-6(5H)-one (27.9 mg, 30.9%) as a yellow solid. ¹H NMR (400 MHz, Chloroform-d) δ 8.45-8.39 (m, 1H), 8.34 (d, J=2.6 Hz, 1H), 8.20 (d, J=2.5 Hz, 1H), 7.96 (d, J=7.8 Hz, 1H), 7.29 (s, 1H), 7.25-7.20 (m, 1H), 7.05-6.94 (m, 2H), 6.92-6.82 (m, 1H), 6.05 (s, 2H), 3.99-2.87 (m, 8H), 2.37 (s, 3H). MS m/z: 499.15 [M+H]⁺.

### 7-(4-(2-Fluoro-6-methylphenyl)piperazin-1-yl)-8-methyl-5-((3-(trifluoromethyl)pyrazin-2-yl)methyl)pyrido[2,3-b]pyrazin-6(5H)-one (97)

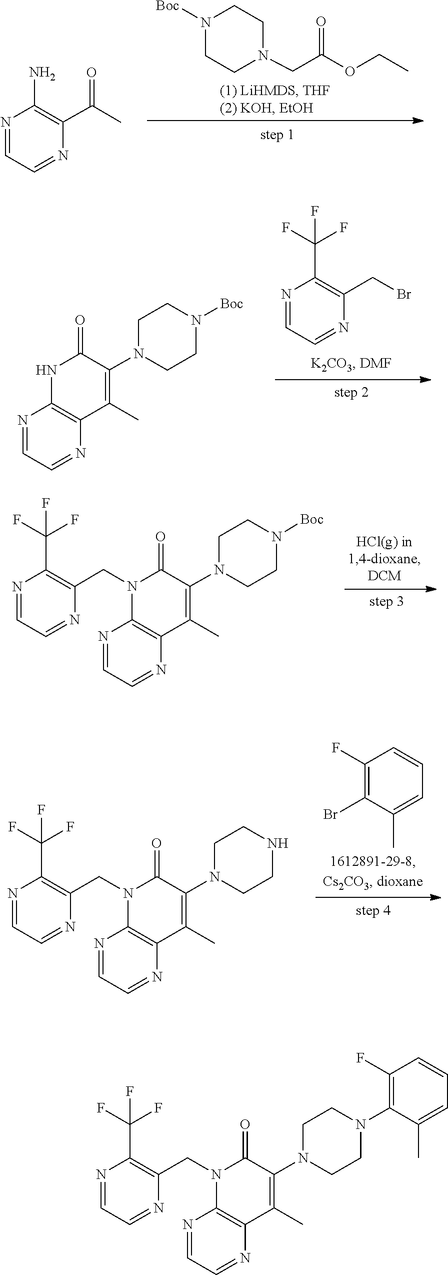

Step 1: tert-butyl 4-(8-methyl-6-oxo-5,6-dihydropyrido[2,3-b]pyrazin-7-yl)piperazine-1-carboxylate: Followed general procedure D using 1-(3-aminopyrazin-2-yl)ethan-1-one (200 mg, 1.45 mmol, 1 equiv) and tert-butyl 4-(2-ethoxy-2-oxoethyl)piperazine-1-carboxylate (436 mg, 1.60 mmol, 1.1 equiv) as the starting materials to give tert-butyl 4-(8-methyl-6-oxo-5,6-dihydropyrido[2,3-b]pyrazin-7-yl)piperazine-1-carboxylate (75 mg, 14%) as a yellow oil. MS m/z: 346 [M+H]⁺.

Step 2: tert-butyl 4-(8-methyl-6-oxo-5-((3-(trifluoromethyl)pyrazin-2-yl)methyl)-5,6-dihydropyrido[2,3-b]pyrazin-7-yl)piperazine-1-carboxylate: Followed general procedure E using tert-butyl 4-(8-methyl-6-oxo-5,6-dihydropyrido[2,3-b]pyrazin-7-yl)piperazine-1-carboxylate (75 mg, 0.217 mmol, 1 equiv) and 2-(bromomethyl)-3-(trifluoromethyl)pyrazine (52.3 mg, 0.217 mmol, 1 equiv) as the starting materials to give tert-butyl 4-(8-methyl-6-oxo-5-((3-(trifluoromethyl)pyrazin-2-yl)methyl)-5,6-dihydropyrido[2,3-b]pyrazin-7-yl)piperazine-1-carboxylat (70 mg, 63%) as a yellow oil. MS m/z: 506 [M+H]⁺.

Step 3: 8-methyl-7-(piperazin-1-yl)-5-((3-(trifluoromethyl)pyrazin-2-yl)methyl)pyrido[2,3-b]pyrazin-6(5H)-one: Followed general procedure F using tert-butyl 4-(4-methoxy-6-oxo-5,6-dihydropyrido[3,2-d]pyrimidin-7-yl)piperidine-1-carboxylate (70 mg, 0.138 mmol, 1 equiv) as the starting material to give the crude product 8-methyl-7-(piperazin-1-yl)-5-((3-(trifluoromethyl)pyrazin-2-yl)methyl)pyrido[2,3-b]pyrazin-6(5H)-one (50 mg) as a yellow solid. MS m/z: 406 [M+H]⁺.

Step 4: 7-(4-(2-fluoro-6-methylphenyl)piperazin-1-yl)-8-methyl-5-((3-(trifluoromethyl)pyrazin-2-yl)methyl)pyrido[2,3-b]pyrazin-6(5H)-one: Followed general procedure G using tert-butyl 4-(4-methoxy-6-oxo-5-((3-(trifluoromethyl)pyrazin-2-yl)methyl)-5,6-dihydropyrido[3,2-d]pyrimidin-7-yl)piperidine-1-carboxylate (50 mg, 0.123 mmol, 1 equiv) and 2-bromo-1-fluoro-3-methylbenzene (34.97 mg, 0.184 mmol, 1.5 equiv) as the starting materials to give 7-(4-(2-fluoro-6-methylphenyl)piperazin-1-yl)-8-methyl-5-((3-(trifluoromethyl)pyrazin-2-yl)methyl)pyrido[2,3-b]pyrazin-6(5H)-one (27.7 mg, 43%) as a yellow solid. ¹H NMR (400 MHz, Chloroform-d) δ 8.52-8.49 (m, 1H), 8.48 (d, J=2.4 Hz, 1H), 8.46-8.42 (m, 1H), 8.24 (d, J=2.3 Hz, 1H), 7.04-6.95 (m, 2H), 6.93-6.85 (m, 1H), 6.06 (s, 2H), 3.70-3.03 (m, 8H), 2.71 (s, 3H), 2.38 (s, 3H). MS m/z: 514.15 [M+H]⁺.

### 5-((3-(Difluoromethoxy)pyridin-2-yl)methyl)-7-(1-(2-fluoro-6-methylphenyl)piperidin-4-yl)pyrido[2,3-b]pyrazin-6(5H)-one (98)

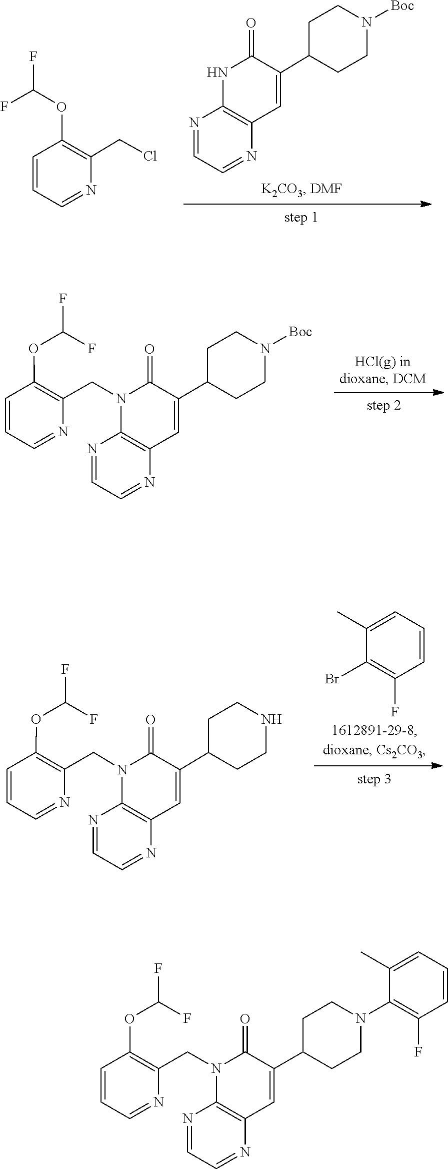

Step 1: tert-butyl 4-(5-((3-(difluoromethoxy)pyridin-2-yl)methyl)-6-oxo-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (79.6 mg, 0.241 mmol, 1 equiv) and 2-(chloromethyl)-3-(difluoromethoxy)pyridine (70 mg, crude) as the starting materials to give tert-butyl 4-(5-((3-(difluoromethoxy)pyridin-2-yl)methyl)-6-oxo-5,6-dihydropyrido[2,3-b]pyrazin-7-yl)piperidine-1-carboxylate (80 mg, 68%) as an off-white solid. MS m/z: 488 [M+H]⁺.

Step 2: 5-((3-(difluoromethoxy)pyridin-2-yl)methyl)-7-(piperidin-4-yl)pyrido[2,3-b]pyrazin-6(5H)-one: Followed general procedure F using tert-butyl 4-(5-((3-(difluoromethoxy)pyridin-2-yl)methyl)-6-oxo-5,6-dihydropyrido[2,3-b]pyrazin-7-yl)piperidine-1-carboxylate (80 mg, 0.164 mmol, 1 equiv) as the starting material to give the crude product 5-((3-(difluoromethoxy)pyridin-2-yl)methyl)-7-(piperidin-4-yl)pyrido[2,3-b]pyrazin-6(5H)-one (45 mg) as an off-white solid. MS m/z: 388 [M+H]⁺.

Step 3: 5-((3-(difluoromethoxy)pyridin-2-yl)methyl)-7-(1-(2-fluoro-6-methylphenyl)piperidin-4-yl)pyrido[2,3-b]pyrazin-6(5H)-one: Followed general procedure G using 5-((3-(difluoromethoxy)pyridin-2-yl)methyl)-3-methyl-7-(piperidin-4-yl)pyrido[2,3-b]pyrazin-6(5H)-one (45 mg, 0.116 mmol, 1 equiv) and 3-bromo-5-fluoro-2-methylpyridine (32.9 mg, 0.174 mmol, 1.5 equiv) as the starting materials to give 5-((3-(difluoromethoxy)pyridin-2-yl)methyl)-7-(1-(2-fluoro-6-methylphenyl)piperidin-4-yl)pyrido[2,3-b]pyrazin-6(5H)-one (10.1 mg, 16.6%) as a brown solid. ¹H NMR (400 MHz, Chloroform-d) δ 8.48 (dd, J=2.4, 0.8 Hz, 1H), 8.39 (d, J=2.4, 0.8 Hz, 1H), 8.20 (d, J=4.8, 1.2 Hz, 1H), 7.89 (s, 1H), 7.51 (d, 1H), 7.20-7.15 (m, 1H), 7.03-6.96 (m, 2H), 6.93-6.53 (m, 2H), 5.92 (s, 2H), 3.37 (t, J=11.6 Hz, 2H), 3.24-3.10 (m, 3H), 2.40 (s, 3H), 2.08-2.02 (m, 2H), 1.92 (s, 2H). MS m/z: 496.15 [M+H]⁺.

### 7-(cis-4-(2-Fluoro-6-methylphenyl)cyclohexyl)-3-methyl-5-((3-(trifluoromethyl)pyrazin-2-yl)methyl)pyrido[2,3-b]pyrazin-6(5H)-one (99a); 7-(trans-4-(2-Fluoro-6-methylphenyl)cyclohexyl)-3-methyl-5-((3-(trifluoromethyl)pyrazin-2-yl)methyl)pyrido[2,3-b]pyrazin-6(5H)-one (99b)

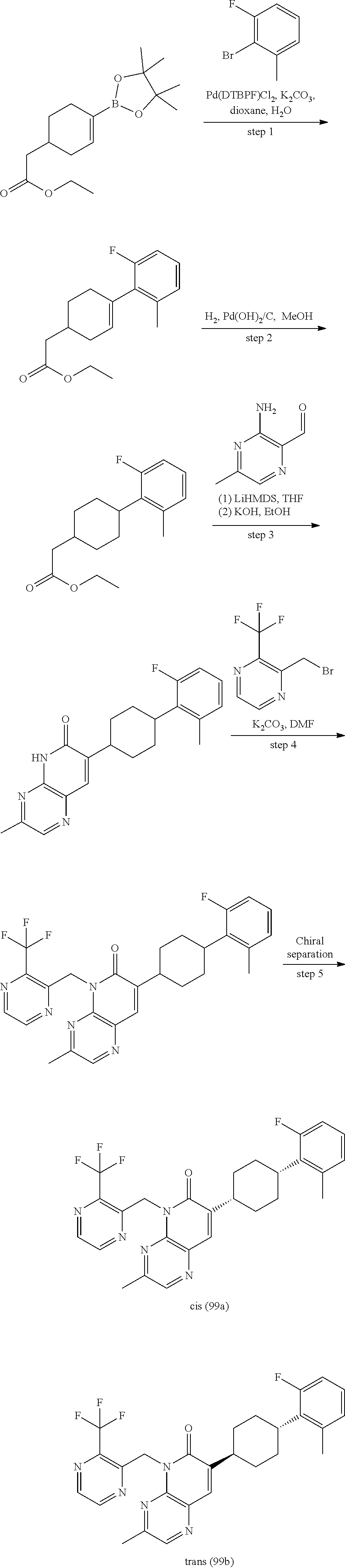

Step 1: ethyl 2-(2′-fluoro-6′-methyl-2,3,4,5-tetrahydro-[1,1′-biphenyl]-4-yl)acetate: Followed general procedure U using ethyl 2-(4-(4,4,5,5-tetramethyl-1,3,2-dioxaborolan-2-yl)cyclohex-3-en-1-yl)acetate (500 mg, 1.70 mmol, 1 equiv) and 2-bromo-1-fluoro-3-methylbenzene (385 mg, 2.04 mmol, 1.2 equiv) as the starting materials to give ethyl 2-(2′-fluoro-6′-methyl-2,3,4,5-tetrahydro-[1,1′-biphenyl]-4-yl)acetate (440 mg, 93%) as a yellow oil. MS m/z: 277[M+H]⁺.

Step 2: ethyl 2-(4-(2-fluoro-6-methylphenyl)cyclohexyl)acetate: Followed general procedure K using ethyl 2-(2′-fluoro-6′-methyl-2,3,4,5-tetrahydro-[1,1′-biphenyl]-4-yl)acetate (200 mg, 0.724 mmol, 1 equiv) as the starting material to give the crude product ethyl 2-(4-(2-fluoro-6-methylphenyl)cyclohexyl)acetate (190 mg, 94%) as a colorless oil. MS m/z: 279 [M+H]⁺.

Step 3: 7-(4-(2-fluoro-6-methylphenyl)cyclohexyl)-3-methylpyrido[2,3-b]pyrazin-6(5H)-one: Followed general procedure D using 2-(4-(2-fluoro-6-methylphenyl)cyclohexyl)acetate (190 mg, 0.683 mmol, 1 equiv) and 3-amino-5-methylpyrazine-2-carbaldehyde (93.6 mg, 0.683 mmol, 1 equiv) as the starting materials to give 7-(4-(2-fluoro-6-methylphenyl)cyclohexyl)-3-methylpyrido[2,3-b]pyrazin-6(5H)-one (100 mg, 41%) as a yellow solid. MS m/z: 352 [M+H]⁺.

Step 4: 7-(4-(2-fluoro-6-methylphenyl)cyclohexyl)-3-methyl-5-((3-(trifluoromethyl)pyrazin-2-yl)methyl)pyrido[2,3-b]pyrazin-6(5H)-one: Followed general procedure E using 7-(4-(2-fluoro-6-methylphenyl)cyclohexyl)-3-methylpyrido[2,3-b]pyrazin-6(5H)-one (100 mg, 0.285 mmol, 1 equiv) and 2-(bromomethyl)-3-(trifluoromethyl)pyrazine (68.5 mg, 0.285 mmol, 1 equiv) as the starting materials to give the crude product 7-(4-(2-fluoro-6-methylphenyl)cyclohexyl)-3-methyl-5-((3-(trifluoromethyl)pyrazin-2-yl)methyl)pyrido[2,3-b]pyrazin-6(5H)-one (80 mg, 54%) as a yellow solid. The crude product was purified by Prep-HPLC with the following conditions Column: (Column: YMC-Actus Triart C18 ExRS, 30*150 mm, 5 m; Mobile Phase A: Water (10 mmol/L NH₄HCO₃+0.1% NH₃·H₂O), Mobile Phase B: ACN; Flow rate: 60 mL/min; Gradient: 72% B to 85% B in 9 min, 85% B; Wave Length: 254/220 nm; RT1 (min): 10.42/11.4; Number Of Runs: 0) to afford 7-((1s,4s)-4-(2-Fluoro-6-methylphenyl)cyclohexyl)-3-methyl-5-((3-(trifluoromethyl)pyrazin-2-yl)methyl)pyrido[2,3-b]pyrazin-6(5H)-one (21 mg, 28.6%) as a white solid and 7-((1r,4r)-4-(2-Fluoro-6-methylphenyl)cyclohexyl)-3-methyl-5-((3-(trifluoromethyl)pyrazin-2-yl)methyl)pyrido[2,3-b]pyrazin-6(5H)-one (23.7 mg, 32.3%) as a white solid.

7-(cis-4-(2-fluoro-6-methylphenyl)cyclohexyl)-3-methyl-5-((3-(trifluoromethyl)pyrazin-2-yl)methyl)pyrido[2,3-b]pyrazin-6(5H)-one (99a)¹H NMR (400 MHz, Chloroform-d) δ 8.49 (s, 1H), 8.45 (s, 1H), 8.38 (s, 1H), 8.21 (s, 1H), 7.05-7.00 (m, 1H), 6.94-6.91 (m, 1H), 6.86-6.79 (m, 1H), 6.08 (s, 2H), 3.45 (t, 1H, J=6.2 Hz), 2.94 (t, 1H, J=12.2 Hz) 2.52 (s, 3H), 2.37 (s, 3H), 2.29-2.19 (m, 4H), 2.01-1.94 (m, 2H), 1.70-1.63 (d, 2H). MS m/z: 512.15 [M+H]⁺

7-(trans-4-(2-fluoro-6-methylphenyl)cyclohexyl)-3-methyl-5-((3-(trifluoromethyl)pyrazin-2-yl)methyl)pyrido[2,3-b]pyrazin-6(5H)-one (99b)¹H NMR (400 MHz, Chloroform-d) δ 8.48 (dd, J=17.9, 2.4 Hz, 2H), 8.34 (s, 1H), 7.83 (s, 1H), 7.08-7.01 (m, 1H), 6.95-6.90 (m, 1H), 6.89-6.82 (m, 1H), 6.08 (s, 2H), 3.13 (t, 1H, J=12.1 Hz, 1H), 2.89 (t, 1H, J=12.4 Hz, 1H), 2.51 (s, 3H), 2.38 (s, 3H), 2.23-2.12 (m, 4H), 1.89-1.83 (m, 2H), 1.59-1.49 (m, 2H). MS m/z: 512.20 [M+H]⁺

### 7-(1-(2-Fluoro-6-methylphenyl)piperidin-4-yl)-3-methyl-5-((2-(trifluoromethoxy)pyridin-3-yl)methyl)pyrido[2,3-b]pyrazin-6(5H)-one (100)

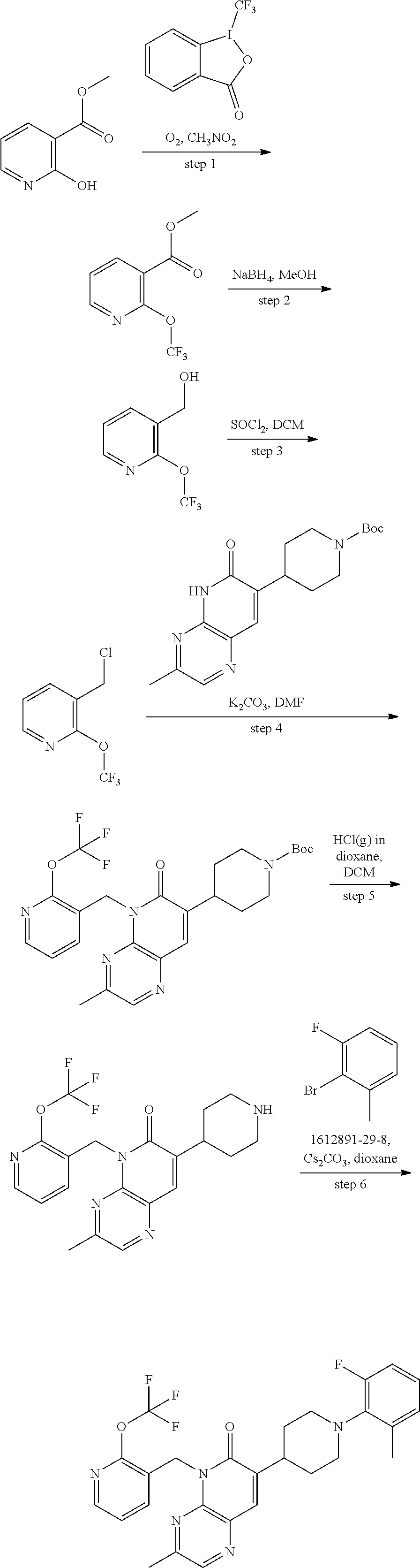

Step 1: methyl 2-(trifluoromethoxy)nicotinate: Followed general procedure AC using methyl 2-hydroxynicotinate (100 mg, 0.653 mmol, 1 equiv) and 1-(trifluoromethyl)-1lambda3,2-benziodaoxol-3-one (619 mg, 1.96 mmol, 3 equiv) as the starting materials to give methyl 2-(trifluoromethoxy)nicotinate (50 mg, 34.6%) as a colorless oil. MS m/z: 222 [M+H]⁺.

Step 2: (2-(trifluoromethoxy)pyridin-3-yl)methanol: Followed general procedure I using methyl 2-(trifluoromethoxy)nicotinate (120 mg, 0.543 mmol, 1 equiv) as the starting material to give (2-(trifluoromethoxy)pyridin-3-yl)methanol (70 mg, 66%) as a colorless oil. MS m/z: 194 [M+H]⁺.

Step 3: 3-(chloromethyl)-2-(trifluoromethoxy)pyridine: Followed general procedure J using (2-(trifluoromethoxy)pyridin-3-yl)methanol (100 mg, 0.474 mmol, 1 equiv) as the starting material to give the crude product 3-(chloromethyl)-2-(trifluoromethoxy)pyridine (100 mg) as a yellow oil. MS m/z: 212 [M+H]⁺.

Step 4: tert-butyl 4-(3-methyl-6-oxo-5-((2-(trifluoromethoxy)pyridin-3-yl)methyl)-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.29 mmol, 1 equiv) and 3-(chloromethyl)-2-(trifluoromethoxy)pyridine (92 mg, 0.436 mmol, 1.5 equiv) as the starting materials to give tert-butyl 4-(3-methyl-6-oxo-5-((2-(trifluoromethoxy)pyridin-3-yl)methyl)-5,6-dihydropyrido[2,3-b]pyrazin-7-yl)piperidine-1-carboxylate (80 mg, 53%) as a yellow solid. MS m/z: 520 [M+H]⁺.

Step 5: 3-methyl-7-(piperidin-4-yl)-5-((2-(trifluoromethoxy)pyridin-3-yl)methyl)pyrido[2,3-b]pyrazin-6(5H)-one: Followed general procedure F using tert-butyl 4-(3-methyl-6-oxo-5-((2-(trifluoromethoxy)pyridin-3-yl)methyl)-5,6-dihydropyrido[2,3-b]pyrazin-7-yl)piperidine-1-carboxylate (80 mg, 0.154 mmol, 1 equiv) as the starting material to give the crude product 3-methyl-7-(piperidin-4-yl)-5-((2-(trifluoromethoxy)pyridin-3-yl)methyl)pyrido[2,3-b]pyrazin-6(5H)-one (70 mg) as a yellow oil. MS m/z: 420 [M+H]⁺.

Step 6: 7-(1-(2-fluoro-6-methylphenyl)piperidin-4-yl)-3-methyl-5-((2-(trifluoromethoxy)pyridin-3-yl)methyl)pyrido[2,3-b]pyrazin-6(5H)-one: Followed general procedure G using 3-methyl-7-(piperidin-4-yl)-5-((2-(trifluoromethoxy)pyridin-3-yl)methyl)pyrido[2,3-b]pyrazin-6(5H)-one (80 mg, 0.191 mmol, 1 equiv) and 2-bromo-1-fluoro-3-methylbenzene (54 mg, 0.286 mmol, 1.5 equiv) as the starting materials to give 7-(1-(2-fluoro-6-methylphenyl)piperidin-4-yl)-3-methyl-5-((2-(trifluoromethoxy)pyridin-3-yl)methyl)pyrido[2,3-b]pyrazin-6(5H)-one (66.8 mg, 65.2%) as a light yellow solid. ¹H NMR (400 MHz, Chloroform-d) δ 8.37 (s, 1H), 8.19 (dd, J=4.8, 2.0 Hz, 1H), 7.84 (s, 1H), 7.46 (dd, J=7.6, 2.0 Hz, 1H), 7.09 (dd, J=7.6, 4.8 Hz, 1H), 7.02-6.95 (m, 2H), 6.92-6.84 (m, 1H), 5.75 (s, 2H), 3.45-3.28 (m, 2H), 3.22-3.04 (m, 3H), 2.59 (s, 3H), 2.37 (s, 3H), 2.10-1.97 (m, 2H), 1.90-1.77 (m, 2H). MS m/z: 528.2 [M+H]⁺.

### 7-(4-(2-Fluoro-6-methylphenyl)piperidin-1-yl)-3-methyl-5-((3-(trifluoromethyl)pyridin-2-yl)methyl)pyrido[2,3-b]pyrazin-6(5H)-one (101)

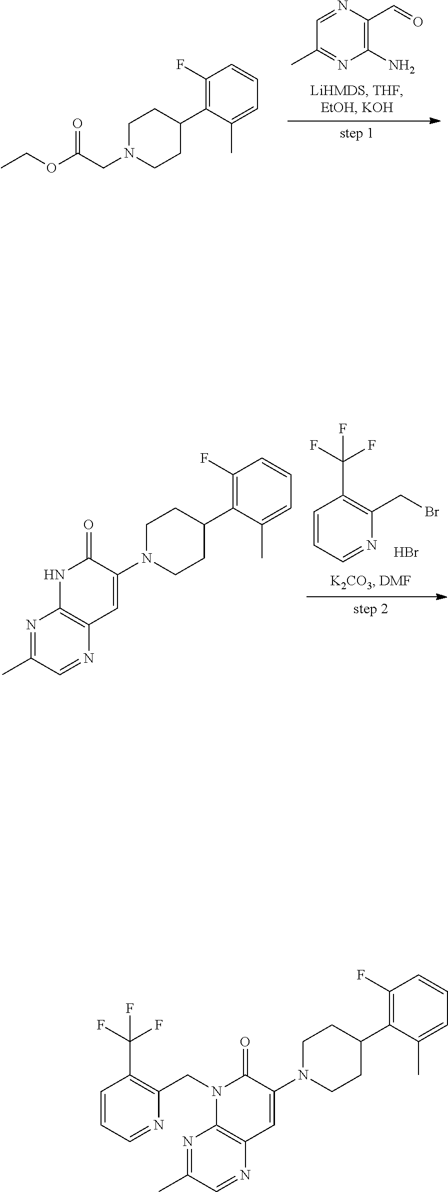

Step 1: 7-(4-(2-fluoro-6-methylphenyl)piperidin-1-yl)-3-methylpyrido[2,3-b]pyrazin-6(5H)-one: Followed general procedure D using ethyl 2-(4-(2-fluoro-6-methylphenyl)piperidin-1-yl)acetate (320 mg, 1.14 mmol, 1 equiv) and 3-amino-5-methylpyrazine-2-carbaldehyde (172 mg, 1.26 mmol, 1.1 equiv) as the starting materials to give 7-(4-(2-fluoro-6-methylphenyl)piperidin-1-yl)-3-methylpyrido[2,3-b]pyrazin-6(5H)-one (115 mg, 28%) as a yellow solid. MS m/z: 353 [M+H]⁺.

Step 2: 7-(4-(2-fluoro-6-methylphenyl)piperidin-1-yl)-3-methyl-5-((3-(trifluoromethyl)pyridin-2-yl)methyl)pyrido[2,3-b]pyrazin-6(5H)-one: Followed general procedure E using 7-(4-(2-fluoro-6-methylphenyl)piperidin-1-yl)-3-methylpyrido[2,3-b]pyrazin-6(5H)-one (80 mg, 0.227 mmol, 1 equiv) and 2-(bromomethyl)-3-(trifluoromethyl)pyridine hydrobromide (80.1 mg, 0.25 mmol, 1.1 equiv) as the starting materials to give 7-(4-(2-fluoro-6-methylphenyl)piperidin-1-yl)-3-methyl-5-((3-(trifluoromethyl)pyridin-2-yl)methyl)pyrido[2,3-b]pyrazin-6(5H)-one (52.5 mg, 42.5%) as a yellow solid. ¹H NMR (400 MHz, Chloroform-d) δ 8.43 (d, J=4.8 Hz, 1H), 8.23 (s, 1H), 7.97-7.94 (m, 1H), 7.24-7.20 (m, 2H), 7.08-7.03 (m, 1H), 6.93 (d, J=7.6 Hz, 1H), 6.88-6.83 (m, 1H), 6.04 (s, 2H), 4.13 (d, J=12.0 Hz, 2H), 3.05-2.97 (m, 1H), 2.81 (t, J=12.0 Hz, 2H), 2.50-2.44 (m, 5H), 2.39 (s, 3H), 1.77 (d, J=13.2 Hz, 2H). MS m/z: 512.15 [M+H]⁺.

### 6-(1-(2-Fluoro-6-methylphenyl)piperidin-4-yl)-2-methyl-8-((3-(trifluoromethoxy)pyridin-2-yl)methyl)pyrido[2,3-d]pyrimidin-7(8H)-one (102)

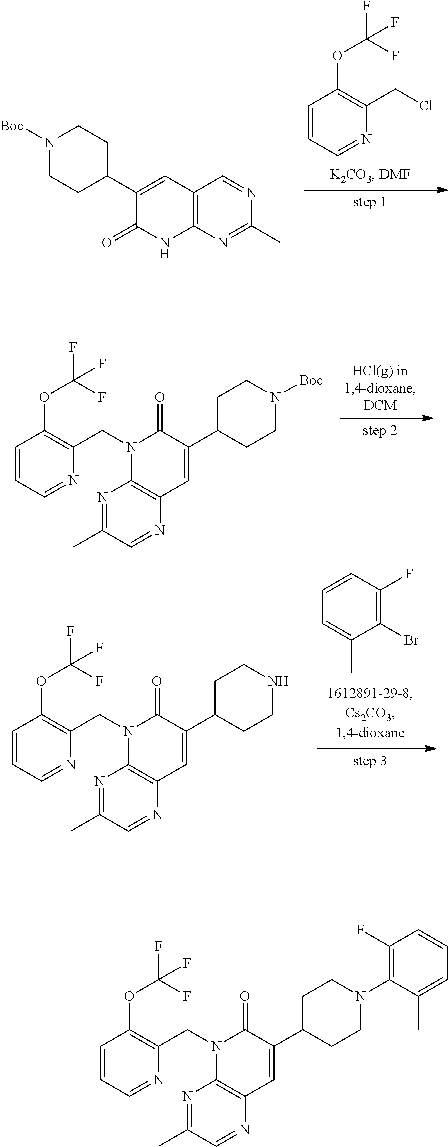

Step 1: tert-butyl 4-(2-methyl-7-oxo-8-((3-(trifluoromethoxy)pyridin-2-yl)methyl)-7,8-dihydropyrido[2,3-d]pyrimidin-6-yl)piperidine-1-carboxylate: Followed general procedure E using tert-butyl 4-(2-methyl-7-oxo-7,8-dihydropyrido[2,3-d]pyrimidin-6-yl)piperidine-1-carboxylate (75 mg, 0.218 mmol, 1 equiv) and 2-(chloromethyl)-3-(trifluoromethoxy)pyridine (55.2 mg, 0.262 mmol, 1.2 equiv) as the starting materials to give tert-butyl 4-(2-methyl-7-oxo-8-((3-(trifluoromethoxy)pyridin-2-yl)methyl)-7,8-dihydropyrido[2,3-d]pyrimidin-6-yl)piperidine-1-carboxylate (94 mg, 83%) as a white solid. MS m/z: 520 [M+H]⁺.

Step 2: 2-methyl-6-(piperidin-4-yl)-8-((3-(trifluoromethoxy)pyridin-2-yl)methyl)pyrido[2,3-d]pyrimidin-7(8H)-one: Followed general procedure F using tert-butyl 4-(2-methyl-7-oxo-8-((3-(trifluoromethoxy)pyridin-2-yl)methyl)-7,8-dihydropyrido[2,3-d]pyrimidin-6-yl)piperidine-1-carboxylate (94 mg, 0.181 mmol, 1 equiv) as the starting material to give the crude product 2-methyl-6-(piperidin-4-yl)-8-((3-(trifluoromethoxy)pyridin-2-yl)methyl)pyrido[2,3-d]pyrimidin-7(8H)-one (88 mg) as a light yellow oil. MS m/z: 420 [M+H]⁺.

Step 3: 6-(1-(2-fluoro-6-methylphenyl)piperidin-4-yl)-2-methyl-8-((3-(trifluoromethoxy)pyridin-2-yl)methyl)pyrido[2,3-d]pyrimidin-7(8H)-one: Followed general procedure G using 2-methyl-6-(piperidin-4-yl)-8-((3-(trifluoromethoxy)pyridin-2-yl)methyl)pyrido[2,3-d]pyrimidin-7(8H)-one (88 mg, 0.21 mmol, 1 equiv) and 2-bromo-1-fluoro-3-methylbenzene (47.5 mg, 0.252 mmol, 1.2 equiv) as the starting materials to give 6-(1-(2-fluoro-6-methylphenyl)piperidin-4-yl)-2-methyl-8-((3-(trifluoromethoxy)pyridine-2-yl)methyl)pyrido[2,3-d]pyrimidin-7(8H)-one (37.3 mg, 33.5%) as a white solid. ¹H NMR (400 MHz, Chloroform-d) δ 8.79 (s, 1H), 8.28-8.26 (m, 1H), 7.60-7.57 (m, 2H), 7.22-7.19 (m, 1H), 7.00-6.96 (m, 2H), 6.88-6.83 (m, 1H), 5.92 (s, 2H), 3.30 (t, J=11.6 Hz, 2H), 3.13-3.06 (m, 3H), 2.67 (s, 3H), 2.35 (s, 3H), 2.02-1.98 (m, 2H), 1.80-1.73 (m, 2H). MS m/z: 528.2 [M+H]⁺.

### 7-(1-(2-Fluoro-6-methylphenyl)piperidin-4-yl)-3,8-dimethyl-5-((3-methylpyrazin-2-yl)methyl)pyrido[2,3-b]pyrazin-6(5H)-one (103)

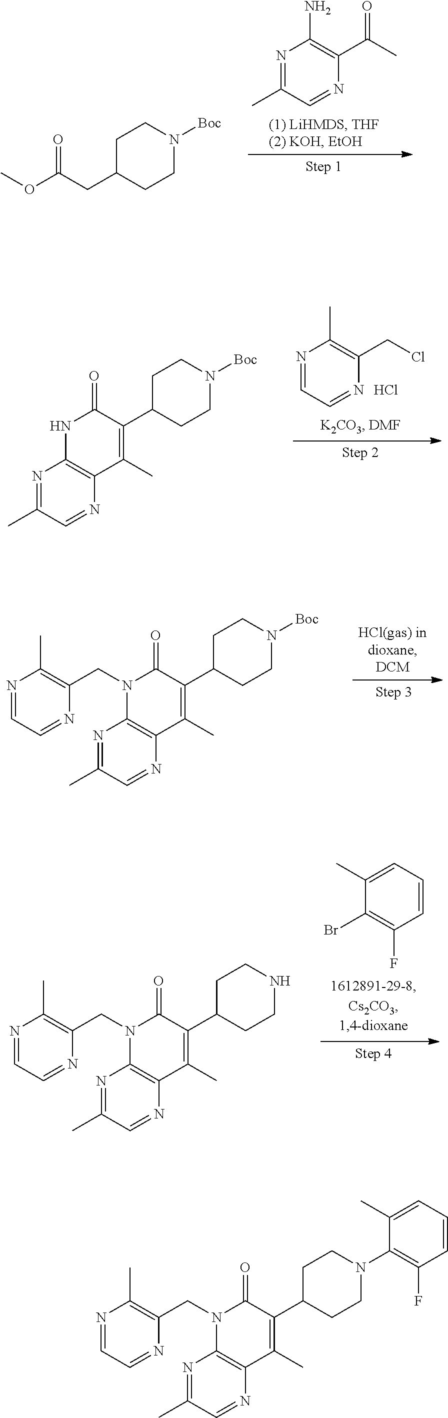

Step 1: tert-butyl 4-(3,8-dimethyl-6-oxo-5,6-dihydropyrido[2,3-b]pyrazin-7-yl)piperidine-1-carboxylate: Followed general procedure D using tert-butyl 4-(2-methoxy-2-oxoethyl)piperidine-1-carboxylate (200 mg, 0.775 mmol, 1.00 equiv) and 1-(3-amino-5-methylpyrazin-2-yl)ethan-1-one (141 mg, 0.93 mmol, 1.2 equiv) as the starting materials to give tert-butyl 4-(3,8-dimethyl-6-oxo-5,6-dihydropyrido[2,3-b]pyrazin-7-yl)piperidine-1-carboxylate (75 mg, 15%) as a yellow oil. MS m/z: 359 [M+H]⁺.

Step 2: tert-butyl 4-(3,8-dimethyl-5-((3-methylpyrazin-2-yl)methyl)-6-oxo-5,6-dihydropyrido[2,3-b]pyrazin-7-yl)piperidine-1-carboxylate: Followed general procedure E using tert-butyl 4-(3,8-dimethyl-6-oxo-5,6-dihydropyrido[2,3-b]pyrazin-7-yl)piperidine-1-carboxylate (75.0 mg, 0.209 mmol, 1 equiv) and 2-(chloromethyl)-3-methylpyrazine hydrochloride (44.9 mg, 0.251 mmol, 1.2 equiv) as the starting materials to give tert-butyl-4-(3,8-dimethyl-5-((3-methylpyrazin-2-yl)methyl)-6-oxo-5,6-dihydropyrido[2,3-b]pyrazin-7-yl)piperidine-1-carboxylate (85 mg, 87%) as a yellow oil. MS m/z: 465 [M+H]⁺.

Step 3: 3,8-dimethyl-5-((3-methylpyrazin-2-yl)methyl)-7-(piperidin-4-yl)pyrido[2,3-b]pyrazin-6(5H)-one: Followed general procedure F using tert-butyl-4-(3,8-dimethyl-5-((3-methylpyrazin-2-yl)methyl)-6-oxo-5,6-dihydropyrido[2,3-b]pyrazin-7-yl)piperidine-1-carboxylate (85 mg, 0.183 mmol, 1.00 equiv) as the starting material to give the crude product 3,8-dimethyl-5-((3-methylpyrazin-2-yl)methyl)-7-(piperidin-4-yl)pyrido[2,3-b]pyrazin-6(5H)-one (60 mg) as a yellow solid. MS m/z: 365 [M+H]⁺.

Step 4: 7-(1-(2-fluoro-6-methylphenyl)piperidin-4-yl)-3,8-dimethyl-5-((3-methylpyrazin-2-yl)methyl)pyrido[2,3-b]pyrazin-6(5H)-one: Followed general procedure G using 3,8-dimethyl-5-((3-methylpyrazin-2-yl)methyl)-7-(piperidin-4-yl)pyrido[2,3-b]pyrazin-6(5H)-one (60.0 mg, 0.164 mmol, 1.00 equiv) and 2-bromo-1-fluoro-3-methylbenzene (46.6 mg, 0.247 mmol, 1.50 equiv) as the starting materials to give 7-(1-(2-fluoro-6-methylphenyl)piperidin-4-yl)-3,8-dimethyl-5-((3-methylpyrazin-2-yl)methyl)pyrido[2,3-b]pyrazin-6(5H)-one (15.0 mg, 19%) as a yellow solid. ¹H NMR (400 MHz, Chloroform-d) δ 8.30 (d, J=21.0 Hz, 2H), 8.12 (d, J=2.6 Hz, 1H), 6.98-6.93 (m, 2H), 6.89-6.82 (m, 1H), 5.82 (s, 2H), 3.26 (t, J=11.1 Hz, 3H), 3.08 (d, J=11.6 Hz, 2H), 2.76 (d, J=18.8 Hz, 6H), 2.69 (d, J=13.8 Hz, 2H), 2.50 (s, 3H), 2.37 (s, 3H). MS m/z: 473.25 [M+H]⁺.

### 7-(4-(2-Fluoro-6-methylphenyl)piperazin-1-yl)-3-methyl-5-((3-methylpyrazin-2-yl)methyl)pyrido[2,3-b]pyrazin-6(5H)-one (104)

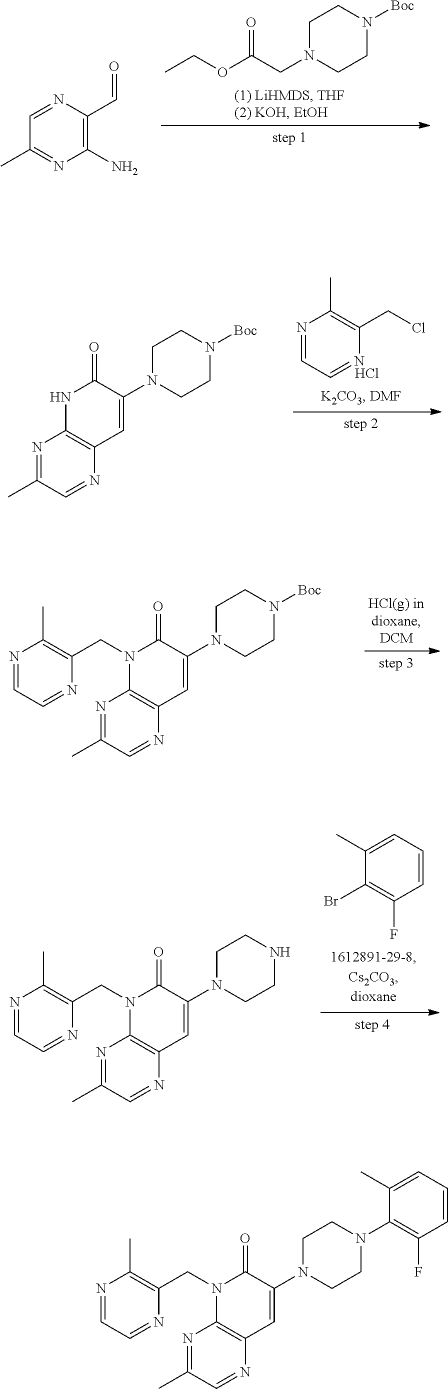

Step 1: tert-butyl 4-(3-methyl-6-oxo-5,6-dihydropyrido[2,3-b]pyrazin-7-yl)piperazine-1-carboxylate: Followed general procedure D using 3-amino-5-methylpyrazine-2-carbaldehyde (100 mg, 0.729 mmol, 1 equiv) and tert-butyl 4-(2-ethoxy-2-oxoethyl)piperazine-1-carboxylate (238 mg, 0.875 mmol, 1.2 equiv) as the starting materials to give tert-butyl 4-(3-methyl-6-oxo-5,6-dihydropyrido[2,3-b]pyrazin-7-yl)piperazine-1-carboxylate (60 mg, 23%) as a yellow oil. MS m/z: 346 [M+H]⁺.

Step 2: tert-butyl 4-(3-methyl-5-((3-methylpyrazin-2-yl)methyl)-6-oxo-5,6-dihydropyrido[2,3-b]pyrazin-7-yl)piperazine-1-carboxylate: Followed general procedure E using tert-butyl 4-(3-methyl-6-oxo-5,6-dihydropyrido[2,3-b]pyrazin-7-yl)piperazine-1-carboxylate (60 mg, 0.173 mmol, 1 equiv) and 2-(chloromethyl)-3-methylpyrazine (37.1 mg, 0.208 mmol, 1.2 equiv) as the starting materials to give tert-butyl 4-(3-methyl-5-((3-methylpyrazin-2-yl)methyl)-6-oxo-5,6-dihydropyrido[2,3-b]pyrazin-7-yl)piperazine-1-carboxylate (60 mg, 76%) as a yellow oil. MS m/z: 452 [M+H]⁺.

Step 3: 3-methyl-5-((3-methylpyrazin-2-yl)methyl)-7-(piperazin-1-yl)pyrido[2,3-b]pyrazin-6(5H)-one: Followed general procedure F using tert-butyl 4-(3-methyl-5-((3-methylpyrazin-2-yl)methyl)-6-oxo-5,6-dihydropyrido[2,3-b]pyrazin-7-yl)piperazine-1-carboxylate (60 mg, 0.133 mmol, 1 equiv) as the starting material to give the crude product 3-methyl-5-((3-methylpyrazin-2-yl)methyl)-7-(piperazin-1-yl)pyrido[2,3-b]pyrazin-6(5H)-one (50 mg) as a yellow oil. MS m/z: 352 [M+H]⁺.

Step 4: 7-(4-(2-fluoro-6-methylphenyl)piperazin-1-yl)-3-methyl-5-((3-methylpyrazin-2-yl)methyl)pyrido[2,3-b]pyrazin-6(5H)-one: Followed general procedure G using 3-methyl-5-((3-methylpyrazin-2-yl)methyl)-7-(piperazin-1-yl)pyrido[2,3-b]pyrazin-6(5H)-one (50 mg, 0.142 mmol, 1 equiv) and 2-bromo-1-fluoro-3-methylbenzene (32.1 mg, 0.170 mmol, 1.2 equiv) as the starting materials to give 7-(4-(2-fluoro-6-methylphenyl)piperazin-1-yl)-3-methyl-5-((3-methylpyrazin-2-yl)methyl)pyrido[2,3-b]pyrazin-6(5H)-one (19.7 mg, 30.1%) as a pink solid. ¹H NMR (400 MHz, Chloroform-d) δ 8.30-8.22 (m, 2H), 8.12 (d, J=2.6 Hz, 1H), 7.21 (s, 1H), 7.04-6.96 (m, 2H), 6.90-6.83 (m, 1H), 5.85 (s, 2H), 3.75-3.08 (m, 8H), 2.79 (s, 3H), 2.50 (s, 3H), 2.37 (s, 3H). MS m/z: 460.2 [M+H]⁺.

### 8-((3-(Difluoromethoxy)pyridin-2-yl)methyl)-6-(1-(2-fluoro-6-methylphenyl)piperidin-4-yl)-2-methylpyrido[2,3-d]pyrimidin-7(8H)-one (105)

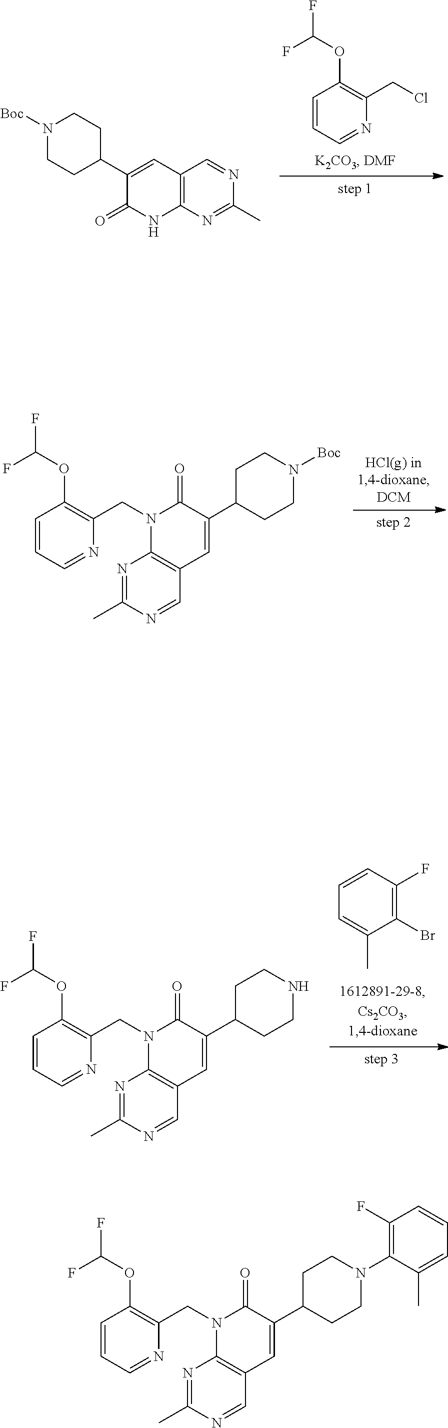

Step 1: tert-butyl 4-(8-((3-(difluoromethoxy)pyridin-2-yl)methyl)-2-methyl-7-oxo-7,8-dihydropyrido[2,3-d]pyrimidin-6-yl)piperidine-1-carboxylate: Followed general procedure E using tert-butyl 4-(2-methyl-7-oxo-7,8-dihydropyrido[2,3-d]pyrimidin-6-yl)piperidine-1-carboxylate (75 mg, 0.218 mmol, 1 equiv) and 2-(chloromethyl)-3-(difluoromethoxy)pyridine (50.5 mg, 0.262 mmol, 1.2 equiv) as the starting materials to give tert-butyl 4-(8-((3-(difluoromethoxy)pyridin-2-yl)methyl)-2-methyl-7-oxo-7,8-dihydropyrido[2,3-d]pyrimidin-6-yl)piperidine-1-carboxylate (64 mg, 58%) as a light yellow solid. MS m/z: 502 [M+H]⁺.

Step 2: 8-((3-(difluoromethoxy)pyridin-2-yl)methyl)-2-methyl-6-(piperidin-4-yl)pyrido[2,3-d]pyrimidin-7(8H)-one: Followed general procedure F using tert-butyl 4-(8-((3-(difluoromethoxy)pyridin-2-yl)methyl)-2-methyl-7-oxo-7,8-dihydropyrido[2,3-d]pyrimidin-6-yl)piperidine-1-carboxylate (64 mg, 0.128 mmol, 1 equiv) as the starting material to give the crude product 8-((3-(difluoromethoxy)pyridin-2-yl)methyl)-2-methyl-6-(piperidin-4-yl)pyrido[2,3-d]pyrimidin-7(8H)-one (67 mg) as a yellow oil. MS m/z: 402 [M+H]⁺.

Step 3: 8-((3-(difluoromethoxy)pyridin-2-yl)methyl)-6-(1-(2-fluoro-6-methylphenyl)piperidin-4-yl)-2-methylpyrido[2,3-d]pyrimidin-7(8H)-one: Followed general procedure G using 8-((3-(difluoromethoxy)pyridin-2-yl)methyl)-2-methyl-6-(piperidin-4-yl)pyrido[2,3-d]pyrimidin-7(8H)-one (67 mg, 0.167 mmol, 1 equiv) and 2-bromo-1-fluoro-3-methylbenzene (37.8 mg, 0.2 mmol, 1.2 equiv) as the starting materials to give 8-((3-(difluoromethoxy)pyridin-2-yl)methyl)-6-(1-(2-fluoro-6-methylphenyl)piperidin-4-yl)-2-methylpyrido[2,3-d]pyrimidin-7(8H)-one (26 mg, 30.4%) as a white solid. ¹H NMR (400 MHz, Chloroform-d) δ 8.79 (s, 1H), 8.21-8.20 (m, 1H), 7.59 (d, J=0.8 Hz, 1H), 7.52-7.48 (m, 1H), 7.18-7.15 (m, 1H), 7.00-6.92 (m, 2H), 6.89-6.56 (m, 2H), 5.89 (s, 2H), 3.34-3.27 (m, 2H), 3.13-3.05 (m, 3H), 2.70 (s, 3H), 2.35 (s, 3H), 2.01-1.97 (m, 2H), 1.80-1.73 (m, 2H). MS m/z: 510.15 [M+H]⁺.

### 7-(1-(2-Fluoro-6-methylphenyl)piperidin-4-yl)-3-methyl-5-((3-methylpyrazin-2-yl)methyl)pyrido[2,3-b]pyrazin-6(5H)-one (106)

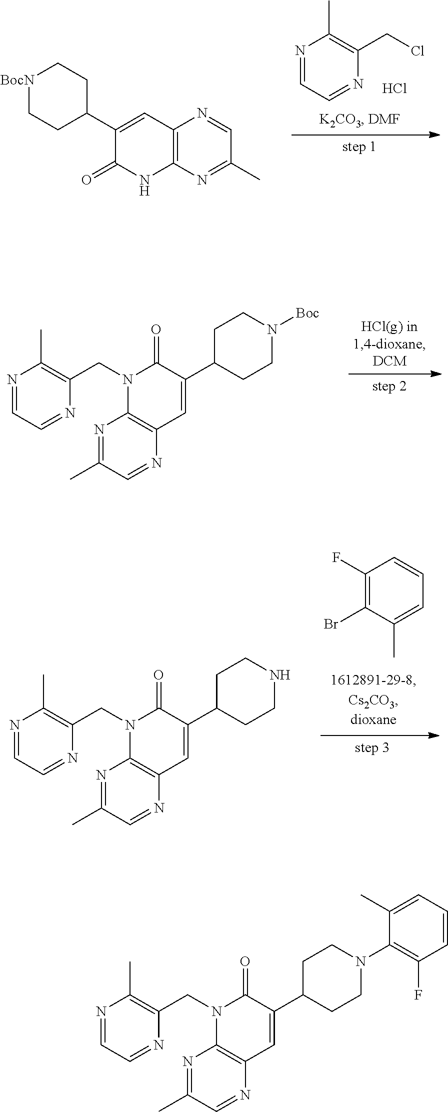

Step 1: tert-butyl 4-(3-methyl-5-((3-methylpyrazin-2-yl)methyl)-6-oxo-5,6-dihydropyrido[2,3-b]pyrazin-7-yl)piperidine-1-carboxy: Followed general procedure E using tert-butyl 4-(3-methyl-6-oxo-5,6-dihydropyrido[2,3-b]pyrazin-7-yl)piperidine-1-carboxylate (80 mg, 0.232 mmol, 1 equiv) and 2-(chloromethyl)-3-methylpyrazine hydrochloride (46 mg, 0.255 mmol, 1.1 equiv) as the starting materials to give tert-butyl 4-(3-methyl-5-((3-methylpyrazin-2-yl)methyl)-6-oxo-5,6-dihydropyrido[2,3-b]pyrazin-7-yl)piperidine-1-carboxylate (70 mg, 66%) as a white solid. MS m/z: 451 [M+H]⁺.

Step 2: 3-methyl-5-((3-methylpyrazin-2-yl)methyl)-7-(piperidin-4-yl)pyrido[2,3-b]pyrazin-6(5H)-one: Followed general procedure F using tert-butyl 4-(3-methyl-5-((3-methylpyrazin-2-yl)methyl)-6-oxo-5,6-dihydropyrido[2,3-b]pyrazin-7-yl)piperidine-1-carboxylate (70 mg, 0.155 mmol, 1 equiv) as the starting material to give the crude product 3-methyl-5-((3-methylpyrazin-2-yl)methyl)-7-(piperidin-4-yl)pyrido[2,3-b]pyrazin-6(5H)-one (60 mg) as a white solid. MS m/z: 351 [M+H]⁺.

Step 3: 7-(1-(2-fluoro-6-methylphenyl)piperidin-4-yl)-3-methyl-5-((3-methylpyrazin-2-yl)methyl)pyrido[2,3-b]pyrazin-6(5H)-one: Followed general procedure G using 3-methyl-5-((3-methylpyrazin-2-yl)methyl)-7-(piperidin-4-yl)pyrido[2,3-b]pyrazin-6(5H)-one (60 mg, 0.171 mmol, 1 equiv) and 2-bromo-1-fluoro-3-methylbenzene (25.6 mg, 0.188 mmol, 1.1 equiv) as the starting materials to give 7-(1-(2-fluoro-6-methylphenyl)piperidin-4-yl)-3-methyl-5-((3-methylpyrazin-2-yl)methyl)pyrido[2,3-b]pyrazin-6(5H)-one (40.9 mg, 45.6%) as a yellow solid. ¹H NMR (400 MHz, Chloroform-d) δ 8.34 (s, 1H), 8.28 (d, J=2.6 Hz, 1H), 8.12 (d, J=2.6 Hz, 1H), 7.85 (d, J=1.0 Hz, 1H), 7.00-6.92 (m, 2H), 6.91-6.81 (m, 1H), 5.84 (s, 2H), 3.38-3.27 (m, 2H), 3.20-3.07 (m, 3H), 2.80 (s, 3H), 2.54 (s, 3H), 2.36 (s, 3H), 2.08-1.99 (m, 2H), 1.88-1.74 (m, 2H). MS m/z: 459.2 [M+H]⁺.

### 6-(1-(2-Fluoro-6-methylphenyl)piperidin-4-yl)-2-methyl-8-((3-methylpyrazin-2-yl)methyl)pyrido[2,3-d]pyrimidin-7(8H)-one (107)

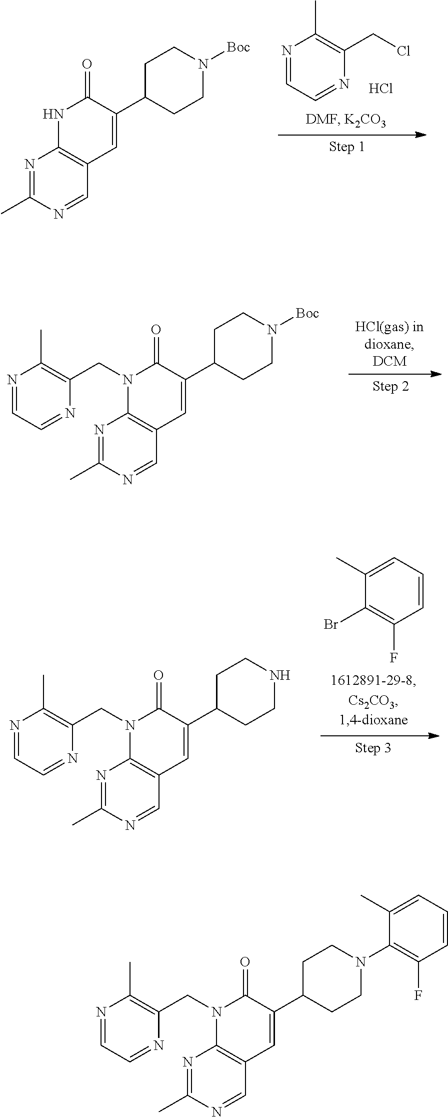

Step 1: tert-butyl 4-(2-methyl-8-((3-methylpyrazin-2-yl)methyl)-7-oxo-7,8-dihydropyrido[2,3-d]pyrimidin-6-yl)piperidine-1-carboxylate: Followed general procedure E using tert-butyl 4-(2-methyl-7-oxo-7,8-dihydropyrido[2,3-d]pyrimidin-6-yl)piperidine-1-carboxylate (90 mg, 0.261 mmol, 1 equiv) and 2-(chloromethyl)-3-methylpyrazine hydrochloride (44.7 mg, 0.313 mmol, 1.2 equiv) as the starting materials to give tert-butyl 4-(2-methyl-8-((3-methylpyrazin-2-yl)methyl)-7-oxo-7,8-dihydropyrido[2,3-d]pyrimidin-6-yl)piperidine-1-carboxylate (100 mg, 84%) as a white solid. MS m/z: 451 [M+H]⁺.

Step 2: 2-methyl-8-((3-methylpyrazin-2-yl)methyl)-6-(piperidin-4-yl)pyrido[2,3-d]pyrimidin-7(8H)-one: Followed general procedure F using tert-butyl 4-(2-methyl-8-((3-methylpyrazin-2-yl)methyl)-7-oxo-7,8-dihydropyrido[2,3-d]pyrimidin-6-yl)piperidine-1-carboxylate (100 mg, 0.222 mmol, 1 equiv) as the starting material to give the crude product 2-methyl-8-((3-methylpyrazin-2-yl)methyl)-6-(piperidin-4-yl)pyrido[2,3-d]pyrimidin-7(8H)-one (50 mg) as a light yellow solid. MS m/z: 351 [M+H]⁺.

Step 3: 6-(1-(2-fluoro-6-methylphenyl)piperidin-4-yl)-2-methyl-8-((3-methylpyrazin-2-yl)methyl)pyrido[2,3-d]pyrimidin-7(8H)-one: Followed general procedure G using 2-methyl-8-((3-methylpyrazin-2-yl)methyl)-6-(piperidin-4-yl)pyrido[2,3-d]pyrimidin-7(8H)-one (50 mg, 0.043 mmol, 1 equiv) and 2-bromo-1-fluoro-3-methylbenzene (12.2 mg, 0.065 mmol, 1.5 equiv) as the starting materials to give 6-(1-(2-fluoro-6-methylphenyl)piperidin-4-yl)-2-methyl-8-((3-methylpyrazin-2-yl)methyl)pyrido[2,3-d]pyrimidin-7(8H)-one (19.8 mg, 30.3%) as a white solid. ¹H NMR (300 MHz, DMSO-d₆) δ 9.02 (s, 1H), 8.35 (d, J=2.6 Hz, 1H), 8.18 (d, J=2.9 Hz, 1H), 7.99 (s, 1H), 7.05-6.94 (m, 3H), 5.72 (s, 2H), 3.20-3.01 (m, 4H), 2.89 (t, J=12.1 Hz, 1H), 2.73 (s, 3H), 2.56 (s, 311), 2.32 (s, 3H), 1.89 (d, J=12.0 Hz, 2H), 1.79-1.64 (in, 211). MS m/z: 459.15 [M+H]⁺.

### 5-((3-Methylpyrazin-2-yl)methyl)-7-(1-(2-(trifluoromethoxy)phenyl)piperidin-4-yl)pyrido[2,3-b]pyrazin-6(5H)-one (108)

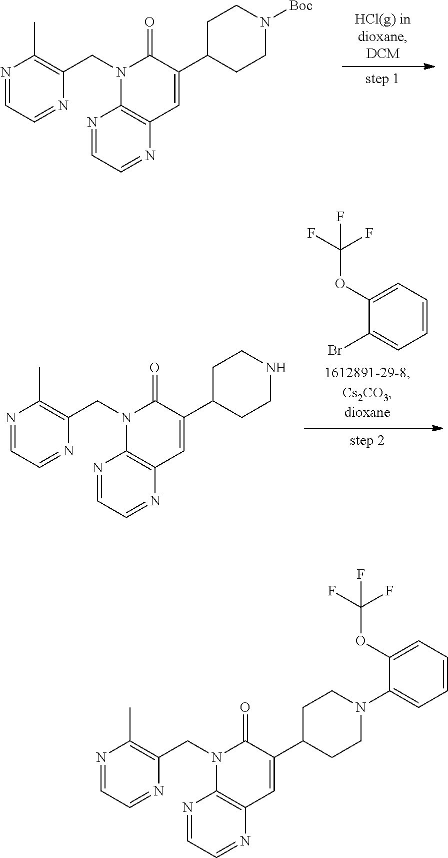 [SMILES: Cc1nccnc1Cn1c(=O)c(C2CCN(C(=O)OC(C)(C)C)CC2)cc2nccnc21 ; FC(F)(F)Oc1ccccc1Br ; Cc1nccnc1Cn1c(=O)c(C2CCNCC2)cc2nccnc21 ; Cc1nccnc1Cn1c(=O)c(C2CCN(c3ccccc3OC(F)(F)F)CC2)cc2nccnc21]

Step 1: 5-((3-methylpyrazin-2-yl)methyl)-7-(piperidin-4-yl)pyrido[2,3-b]pyrazin-6(5H)-one: Followed general procedure F using tert-butyl 4-(5-((3-methylpyrazin-2-yl)methyl)-6-oxo-5,6-dihydropyrido[2,3-b]pyrazin-7-yl)piperidine-1-carboxylate (60 mg, 0.137 mmol, 1 equiv) as the starting material to give the crude product 5-((3-methylpyrazin-2-yl)methyl)-7-(piperidin-4-yl)pyrido[2,3-b]pyrazin-6(5H)-one (40 mg) as a yellow oil. MS m/z: 346 [M+H]⁺.

Step 2: 5-((3-methylpyrazin-2-yl)methyl)-7-(1-(2-(trifluoromethoxy)phenyl)piperidin-4-yl)pyrido[2,3-b]pyrazin-6(5H)-one: Followed general procedure G using 5-((3-methylpyrazin-2-yl)methyl)-7-(piperidin-4-yl)pyrido[2,3-b]pyrazin-6(5H)-one (40 mg, 0.119 mmol, 1 equiv) and 1-bromo-2-(trifluoromethoxy)benzene (42.99 mg, 0.178 mmol, 1.5 equiv) as the starting materials to give 5-((3-methylpyrazin-2-yl)methyl)-7-(1-(2-(trifluoromethoxy)phenyl)piperidin-4-yl)pyrido[2,3-b]pyrazin-6(5H)-one (28.9 mg, 47.9%) as a yellow solid. ¹H NMR (400 MHz, Chloroform-d) δ 8.49 (d, J=2.4 Hz, 1H), 8.37 (d, J=2.4 Hz, 1H), 8.29 (d, J=2.6 Hz, 1H), 8.09 (d, J=2.6 Hz, 1H), 7.93-7.87 (m, 1H), 7.25-7.19 (m, 2H), 7.15-7.07 (m, 1H), 7.10 (d, J=7.9 Hz, 1H), 7.02 (s, 1H), 5.84 (s, 2H), 3.55 (d, J=11.6 Hz, 2H), 3.17 (dd, J=13.8, 10.2 Hz, 1H), 2.93 (s, 2H), 2.78 (s, 3H), 2.11 (d, J=12.3 Hz, 2H), 2.01 (s, 2H). MS m/z: 497.15 [M+H]⁺.

### 5-((3-Methylpyrazin-2-yl)methyl)-7-(1-(o-tolyl)piperidin-4-yl)pyrido[2,3-b]pyrazin-6(5H)-one (109)

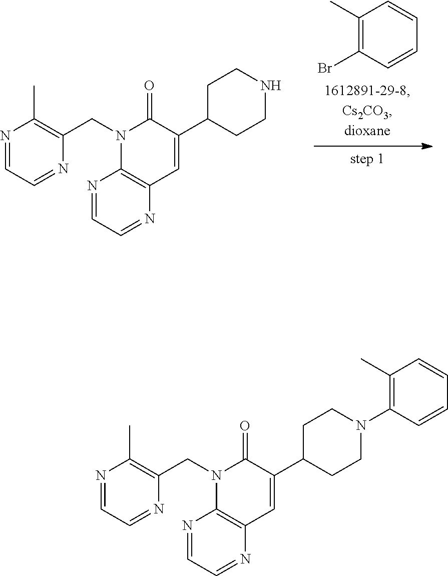

Step 1: 5-((3-methylpyrazin-2-yl)methyl)-7-(1-(o-tolyl)piperidin-4-yl)pyrido[2,3-b]pyrazin-6(5H)-one: Followed general procedure G using 5-((3-methylpyrazin-2-yl)methyl)-7-(piperidin-4-yl)pyrido[2,3-b]pyrazin-6(5H)-one (40 mg, 0.119 mmol, 1 equiv) and 1-bromo-2-methylbenzene (24.4 mg, 0.143 mmol, 1.2 equiv) as the starting materials to give 5-((3-methylpyrazin-2-yl)methyl)-7-(1-(o-tolyl)piperidin-4-yl)pyrido[2,3-b]pyrazin-6(5H)-one (40 mg, 78.3%) as a yellow solid. ¹H NMR (400 MHz, DMSO-d₆) δ 8.60 (d, J=2.4 Hz, 1H), 8.53 (d, J=2.4 Hz, 1H), 8.35 (d, J=2.6 Hz, 1H), 8.16 (d, J=2.6 Hz, 1H), 7.95 (s, 1H), 7.19-7.11 (m, 2H), 7.07 (d, J=7.7 Hz, 1H), 6.98-6.91 (m, 1H), 5.78 (s, 2H), 3.18 (d, J=11.5 Hz, 2H), 3.04-2.93 (m, 1H), 2.80-2.73 (m, 2H), 2.72 (s, 3H), 2.28 (s, 3H), 2.02-1.93 (m, 2H), 1.91-1.79 (m, 2H). MS m/z: 427.15 [M+H]⁺.

### 6-(4-(2-Fluoro-6-methylphenyl)piperazin-1-yl)-2-methyl-8-((3-(trifluoromethoxy)pyridin-2-yl)methyl)pyrido[2,3-d]pyrimidin-7(8H)-one (110)

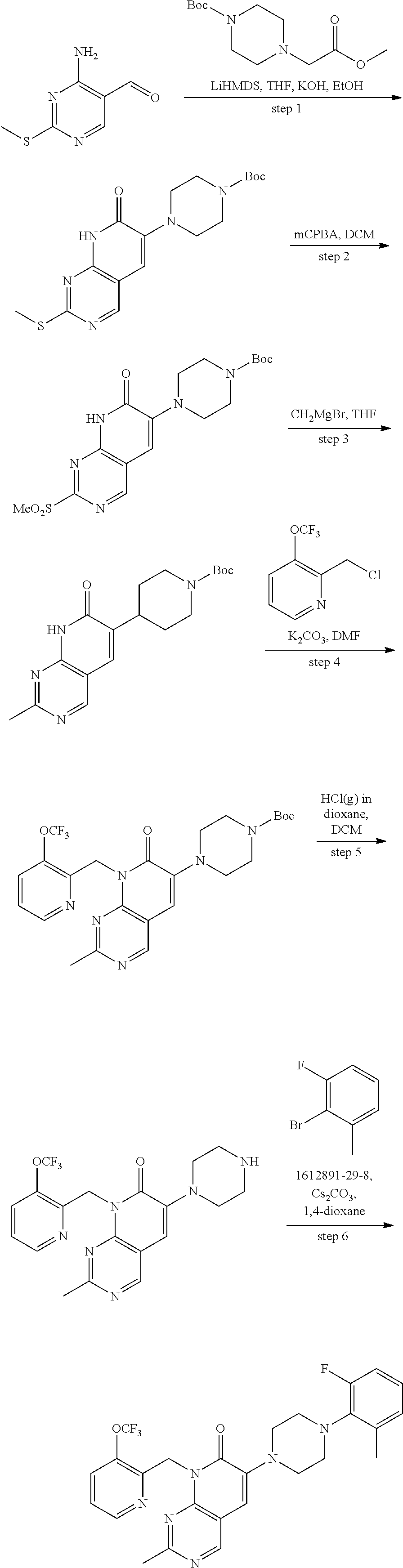

Step 1: tert-butyl 4-(2-(methylthio)-7-oxo-7,8-dihydropyrido[2,3-d]pyrimidin-6-yl)piperazine-1-carboxylate: Followed general procedure D using 3-aminopyrazine-2-carbaldehyde (800 mg, 4.73 mmol, 1 equiv) and tert-butyl 4-(2-ethoxy-2-oxoethyl)piperazine-1-carboxylate (1.47 g, 5.67 mmol, 1.2 equiv) as the starting materials to give tert-butyl 4-(2-(methylthio)-7-oxo-7,8-dihydropyrido[2,3-d]pyrimidin-6-yl)piperazine-1-carboxylate (800 mg, 44.8%) as a yellow solid. MS m/z: 378 [M+H]⁺.

Step 2: tert-butyl 4-(2-(methylsulfonyl)-7-oxo-7,8-dihydropyrido[2,3-d]pyrimidin-6-yl)piperazine-1-carboxylate: Followed general procedure AG using tert-butyl 4-(2-(methylthio)-7-oxo-7,8-dihydropyrido[2,3-d]pyrimidin-6-yl)piperazine-1-carboxylate (800 mg, 2.12 mmol, 1 equiv) as the starting material to give tert-butyl 4-(2-(methylsulfonyl)-7-oxo-7,8-dihydropyrido[2,3-d]pyrimidin-6-yl)piperazine-1-carboxylate (300 mg, 34%) as a white solid. MS m/z: 410 [M+H]⁺.

Step 3: tert-butyl 4-(2-methyl-7-oxo-7,8-dihydropyrido[2,3-d]pyrimidin-6-yl)piperazine-1-carboxylate: Followed general procedure C using tert-butyl 4-(2-(methylsulfonyl)-7-oxo-7,8-dihydropyrido[2,3-d]pyrimidin-6-yl)piperazine-1-carboxylate (300 mg, 0.733 mmol, 1 equiv) as the starting materials to give tert-butyl 4-(2-methyl-7-oxo-7,8-dihydropyrido[2,3-d]pyrimidin-6-yl)piperazine-1-carboxylate (100 mg, 39%) as a yellow solid. MS m/z: 346 [M+H]⁺.

Step 4: tert-butyl 4-(2-methyl-7-oxo-8-((3-(trifluoromethoxy)pyridin-2-yl)methyl)-7,8-dihydropyrido[2,3-d]pyrimidin-6-yl)piperazine-1-carboxylate: Followed general procedure E using tert-butyl 4-(2-methyl-7-oxo-7,8-dihydropyrido[2,3-d]pyrimidin-6-yl)piperazine-1-carboxylate (100 mg, 0.29 mmol, 1 equiv) and 2-(chloromethyl)-3-(trifluoromethoxy)pyridine (67.4 mg, 0.319 mmol, 1.1 equiv) as the starting materials to give tert-butyl 4-(2-methyl-7-oxo-8-((3-(trifluoromethoxy)pyridin-2-yl)methyl)-7,8-dihydropyrido[2,3-d]pyrimidin-6-yl)piperazine-1-carboxylate (50 mg, 33%) as a yellow solid. MS m/z: 521 [M+H]⁺.

Step 5: 2-methyl-6-(piperazin-1-yl)-8-((3-(trifluoromethoxy)pyridin-2-yl)methyl)pyrido[2,3-d]pyrimidin-7(8H)-one: Followed general procedure F using tert-butyl 4-(2-methyl-7-oxo-8-((3-(trifluoromethoxy)pyridin-2-yl)methyl)-7,8-dihydropyrido[2,3-d]pyrimidin-6-yl)piperazine-1-carboxylate (50 mg, 0.096 mmol, 1 equiv) as the starting material to give the crude product 2-methyl-6-(piperazin-1-yl)-8-((3-(trifluoromethoxy)pyridin-2-yl)methyl)pyrido[2,3-d]pyrimidin-7(8H)-one (40 mg) as a white solid. MS m/z: 421 [M+H]⁺.

Step 6: 6-(4-(2-fluoro-6-methylphenyl)piperazin-1-yl)-2-methyl-8-((3-(trifluoromethoxy)pyridin-2-yl)methyl)pyrido[2,3-d]pyrimidin-7(8H)-one: Followed general procedure G using 2-methyl-6-(piperazin-1-yl)-8-((3-(trifluoromethoxy)pyridin-2-yl)methyl)pyrido[2,3-d]pyrimidin-7(8H)-one (40 mg, 0.095 mmol, 1 equiv) and 2-bromo-1-fluoro-3-methyl benzene (19.8 mg, 0.105 mmol, 1.1 equiv) as the starting materials to give 6-(4-(2-fluoro-6-methylphenyl)piperazin-1-yl)-2-methyl-8-((3-(trifluoromethoxy)pyridin-2-yl)methyl)pyrido[2,3-d]pyrimidin-7(8H)-one (46.1 mg, 90.8%) as a yellow solid. ¹H NMR (400 MHz, Chloroform-d) δ 8.72 (s, 1H), 8.30-8.24 (m, 1H), 7.62-7.54 (m, 1H), 7.23-7.16 (m, 1H), 7.05-6.86 (m, 4H), 5.94 (s, 2H), 3.28 (s, 8H), 2.65 (s, 3H), 2.36 (s, 3H). MS m/z: 529.15 [M+H]⁺.

### 7-(1-(2-(Difluoromethoxy)phenyl)piperidin-4-yl)-5-((3-methylpyrazin-2-yl)methyl)pyrido[2,3-b]pyrazin-6(5H)-one (111)

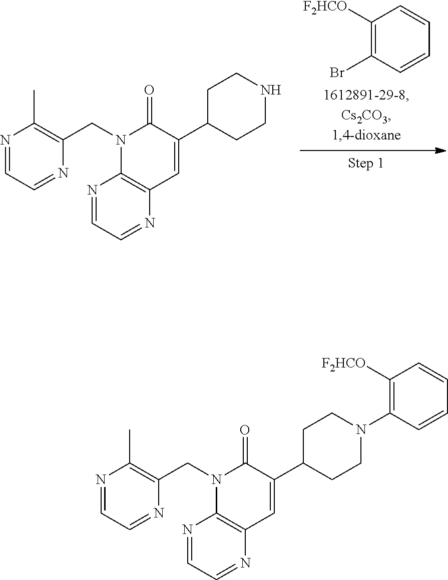 [SMILES: FC(F)Oc1ccccc1Br ; Cc1nccnc1Cn1c(=O)c(C2CCNCC2)cc2nccnc21 ; Cc1nccnc1Cn1c(=O)c(C2CCN(c3ccccc3OC(F)F)CC2)cc2nccnc21]

Step 1: 7-(1-(2-(difluoromethoxy)phenyl)piperidin-4-yl)-5-((3-methylpyrazin-2-yl)methyl)pyrido[2,3-b]pyrazin-6(5H)-one: Followed general procedure G using 5-((3-methylpyrazin-2-yl)methyl)-7-(piperidin-4-yl)pyrido[2,3-b]pyrazin-6(5H)-one (25 mg, 0.074 mmol, 1 equiv) and 1-bromo-2-(difluoromethoxy)benzene (21.7 mg, 0.089 mmol, 1.2 equiv) as the starting materials to give 7-(1-(2-(difluoromethoxy)phenyl)piperidin-4-yl)-5-((3-methylpyrazin-2-yl)methyl)pyrido[2,3-b]pyrazin-6(5H)-one (6.3 mg, 17.4%) as a light yellow solid. ¹H NMR (300 MHz, Chloroform-d) δ 8.51 (d. J=2.4 Hz, 1H), 8.39 (d, J=2.4 Hz, 1H), 8.31 (d, J=2.6 Hz, 1H), 8.12 (d, J=2.6 Hz, 1H), 7.91 (d, J=1.0 Hz, 1H), 7.24-7.00 (m, 4H), 6.67 (t, J=75.8 Hz, 1H), 5.86 (s, 2H), 3.59 (d, J=11.4 Hz, 2H), 3.18 (t, J=12.1 Hz, 1H), 2.89 (d, J=12.1 Hz, 2H), 2.80 (s, 3H), 2.15 (d, J=12.4 Hz, 2H), 1.87 (d, J=12.6 Hz, 2H). MS n/z: 479.15 [M+H]⁺.

### 7-(1-(2-Fluoro-6-methylphenyl)piperidin-4-yl)-3-methoxy-5-((3-methylpyrazin-2-yl)methyl)pyrido[2,3-b]pyrazin-6(5H)-one (112)

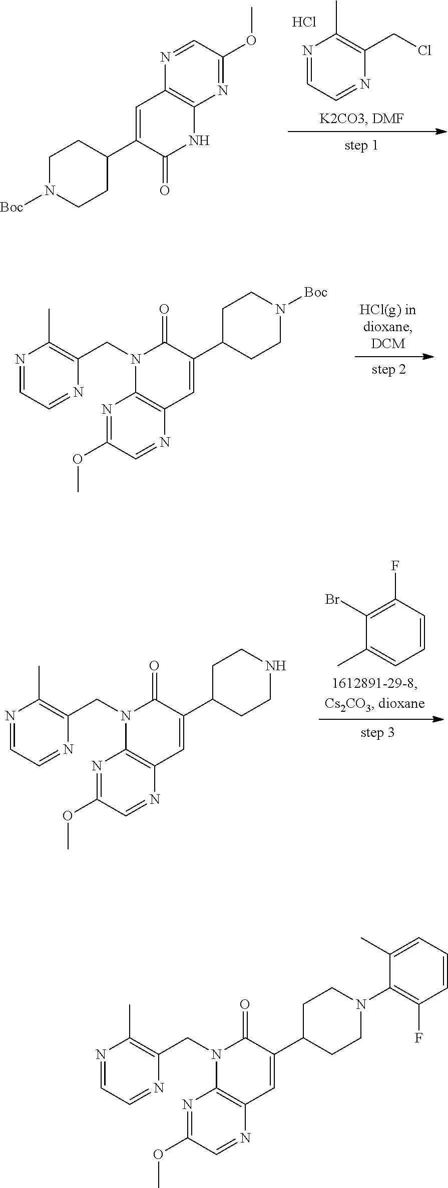

Step 1: tert-butyl 4-(3-methoxy-5-((3-methylpyrazin-2-yl)methyl)-6-oxo-5,6-dihydropyrido[2,3-b]pyrazin-7-yl)piperidine-1-carboxylate: Followed general procedure E using tert-butyl 4-(3-methoxy-6-oxo-5,6-dihydropyrido[2,3-b]pyrazin-7-yl)piperidine-1-carboxylate (100 mg, 0.277 mmol, 1 equiv) and 2-(chloromethyl)-3-methylpyrazine hydrochloride (59.6 mg, 0.332 mmol, 1.2 equiv) as the starting materials to give tert-butyl 4-(3-methoxy-5-((3-methylpyrazin-2-yl)methyl)-6-oxo-5,6-dihydropyrido[2,3-b]pyrazin-7-yl)piperidine-1-carboxylate (95 mg, 73%) as a yellow oil. MS n/z: 467 [M+H]⁺.

Step 2: 3-methoxy-5-((3-methylpyrazin-2-yl)methyl)-7-(piperidin-4-yl)pyrido[2,3-b]pyrazin-6(5H)-one: Followed general procedure F using tert-butyl 4-(3-methoxy-5-((3-methylpyrazin-2-yl)methyl)-6-oxo-5,6-dihydropyrido[2,3-b]pyrazin-7-yl)piperidine-1-carboxylate (95 mg, 0.203 mmol, 1 equiv) as the starting material to give the crude product 7-(1-(2-fluoro-6-methylphenyl)piperidin-4-yl)-3-methoxy-5-((3-methylpyrazin-2-yl)methyl)pyrido[2,3-b]pyrazin-6(5H)-one (75 mg) as a yellow solid. MS m/z: 367 [M+H]⁺.

Step 3: 7-(1-(2-fluoro-6-methylphenyl)piperidin-4-yl)-3-methoxy-5-((3-methylpyrazin-2-yl)methyl)pyrido[2,3-b]pyrazin-6(5H)-one: Followed general procedure G using 7-(1-(2-fluoro-6-methylphenyl)piperidin-4-yl)-3-methoxy-5-((3-methylpyrazin-2-yl)methyl)pyrido[2,3-b]pyrazin-6(5H)-one (75 mg, 0.205 mmol, 1 equiv) and 2-bromo-1-fluoro-3-methylbenzene (46.4 mg, 0.246 mmol, 1.2 equiv) as the starting materials to give 7-(1-(2-fluoro-6-methylphenyl)piperidin-4-yl)-3-methoxy-5-((3-methylpyrazin-2-yl)methyl)pyrido[2,3-b]pyrazin-6(5H)-one (36.3 mg, 35.8%) as a yellow solid. ¹H NMR (400 MHz, Chloroform-d) δ 8.29 (d, J=2.5 Hz, 1H), 8.13 (d, J=2.6 Hz, 1H), 8.10 (s, 1H), 7.85 (s, 1H), 7.00-6.93 (m, 2H), 6.90-6.82 (m, 1H), 5.79 (s, 2H), 3.80 (s, 3H), 3.38-3.28 (m, 2H), 3.18-3.05 (m, 3H), 2.76 (s, 3H), 2.36 (s, 3H), 2.06-1.98 (m, 2H), 1.86-1.73 (m, 2H). MS m/z: 475.15 [M+H]⁺.

### 3-Methyl-7-(1-(3-methylpyridazin-4-yl)piperidin-4-yl)-5-((3-(trifluoromethyl)pyridin-2-yl)methyl)pyrido[2,3-b]pyrazin-6(5H)-one (113)

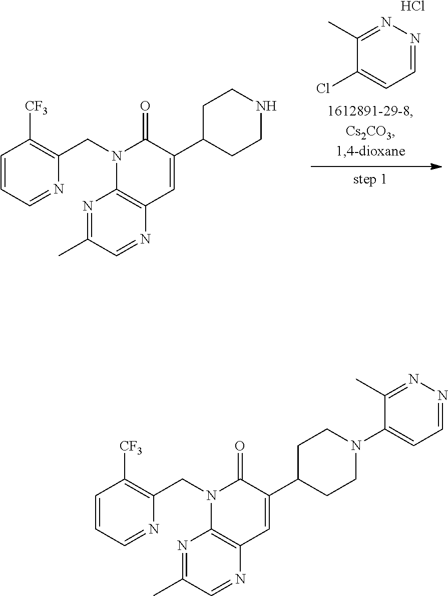

Step 1: 3-methyl-7-(1-(3-methylpyridazin-4-yl)piperidin-4-yl)-5-((3-(trifluoromethyl)pyridin-2-yl)methyl)pyrido[2,3-b]pyrazin-6(5H)-one: Followed general procedure G using 3-methyl-7-(piperidin-4-yl)-5-((3-(trifluoromethyl)pyridin-2-yl)methyl)pyrido[2,3-b]pyrazin-6(5H)-one (40 mg, 0.099 mmol, 1 equiv) and 4-chloro-3-methylpyridazine hydrochloride (18 mg, 0.109 mmol, 1.1 equiv) as the starting materials to give 3-methyl-7-(1-(3-methylpyridazin-4-yl)piperidin-4-yl)-5-((3-(trifluoromethyl)pyridin-2-yl)methyl)pyrido[2,3-b]pyrazin-6(5H)-one (23.4 mg, 45.2%) as an off-white solid. ¹H NMR (400 MHz, Methanol-d₄) δ 8.74 (d, J=5.8 Hz, 1H), 8.45-8.41 (m, 2H), 8.17-8.15 (m, 1H), 7.94 (d, J=0.9 Hz, 1H), 7.43-7.40 (m, 1H), 7.18 (d, J=5.9 Hz, 1H), 6.04 (s, 2H), 3.66 (d, J=12.4 Hz, 2H), 3.20-3.12 (m, 1H), 3.04-2.97 (m, 2H), 2.68 (s, 3H), 2.51 (s, 3H), 2.14 (d, J=12.7 Hz, 2H), 1.99-1.88 (m, 2H). MS m/z: 496.15 [M+H]⁺.

### 3-Methyl-7-(1-(4-methylpyrimidin-5-yl)piperidin-4-yl)-5-((3-(trifluoromethyl)pyridin-2-yl)methyl)pyrido[2,3-b]pyrazin-6(5H)-one (114)

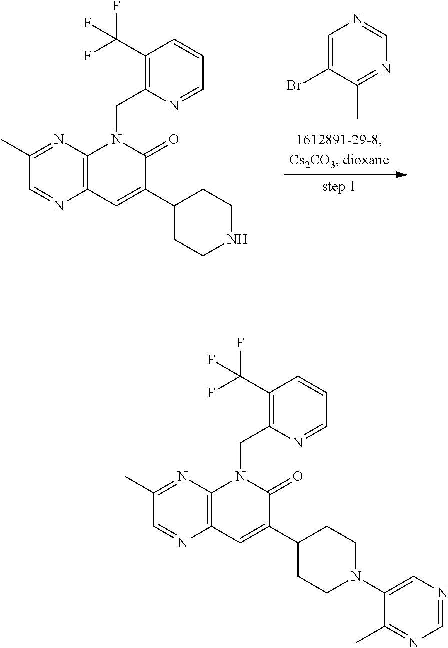

Step 1: 3-methyl-7-(1-(4-methylpyrimidin-5-yl)piperidin-4-yl)-5-((3-(trifluoromethyl)pyridin-2-yl)methyl)pyrido[2,3-b]pyrazin-6(5H)-one: Followed general procedure G using 3-methyl-7-(piperidin-4-yl)-5-((3-(trifluoromethyl)pyridin-2-yl)methyl)pyrido[2,3-b]pyrazin-6(5H)-one (35 mg, 0.087 mmol, 1 equiv) and 5-bromo-4-methylpyrimidine (22.4 mg, 0.13 mmol, 1.5 equiv) as the starting materials to give 3-methyl-7-(1-(4-methylpyrimidin-5-yl)piperidin-4-yl)-5-((3-(trifluoromethyl)pyridin-2-yl)methyl)pyrido[2,3-b]pyrazin-6(5H)-one (10.2 mg, 20.3%) as a white solid. ¹H NMR (400 MHz, Chloroform-d) δ 8.83 (s, 1H), 8.43 (d, J=4.8 Hz, 1H), 8.39 (s, 1H), 8.35 (s, 1H), 7.98 (d, J=7.6 Hz, 1H), 7.85 (s, 1H), 7.23 (s, 1H), 7.24-7.21 (m, 2H), 6.04 (s, 2H), 3.36 (d, J=11.6 Hz, 2H), 3.19 (t, J=12.0 Hz, 1H), 2.98 (t, J=11.6 Hz, 2H), 2.63 (s, 3H), 2.53 (s, 3H), 2.18 (d, J=12.4 Hz, 2H), 1.90-1.85 (m, 2H). MS m/z: 496.15 [M+H]⁺.

### 3-Methyl-7-(1-(4-methylpyridazin-3-yl)piperidin-4-yl)-5-((3-(trifluoromethyl)pyridin-2-yl)methyl)pyrido[2,3-b]pyrazin-6(5H)-one (115)

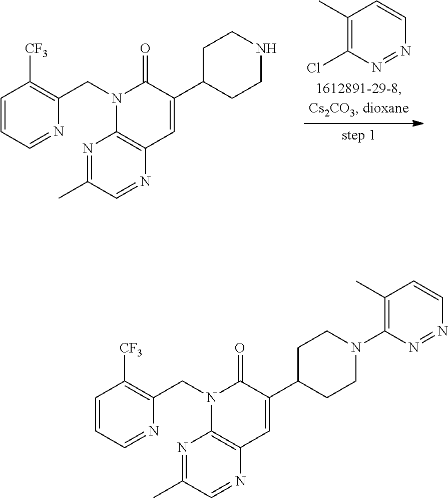

Step 1: 3-methyl-7-(1-(4-methylpyridazin-3-yl)piperidin-4-yl)-5-((3-(trifluoromethyl)pyridin-2-yl)methyl)pyrido[2,3-b]pyrazin-6(5H)-one: Followed general procedure G using 3-methyl-7-(piperidin-4-yl)-5-((3-(trifluoromethyl)pyridin-2-yl)methyl)pyrido[2,3-b]pyrazin-6(5H)-one (40 mg, 0.099 mmol, 1 equiv) and 3-chloro-4-methylpyridazine (15.3 mg, 0.119 mmol, 1.2 equiv) as the starting materials to give 3-methyl-7-(1-(4-methylpyridazin-3-yl)piperidin-4-yl)-5-((3-(trifluoromethyl)pyridin-2-yl)methyl)pyrido[2,3-b]pyrazin-6(5H)-one (40 mg, 78.3%) as a white solid. ¹H NMR (400 MHz, DMSO-d₆) δ 8.71 (d, J=4.7 Hz, 1H), 8.56-8.48 (m, 2H), 8.24 (d, J=7.9 Hz, 1H), 7.92 (s, 1H), 7.52-7.44 (m, 1H), 7.38 (dd, J=4.7, 1.1 Hz, 1H), 5.89 (s, 2H), 3.68 (d, J=12.5 Hz, 2H), 3.11-2.92 (m, 3H), 2.49-2.46 (m, 3H), 2.31 (s, 3H), 2.03-1.95 (m, 2H), 1.92-1.78 (m, 2H). MS m/z: 496.1 [M+H]⁺.

### 7-(1-(2-Fluoro-6-methylphenyl)piperidin-4-yl)-5-((4-(trifluoromethyl)pyrimidin-5-yl)methyl)pyrido[2,3-b]pyrazin-6(5H)-one (116)

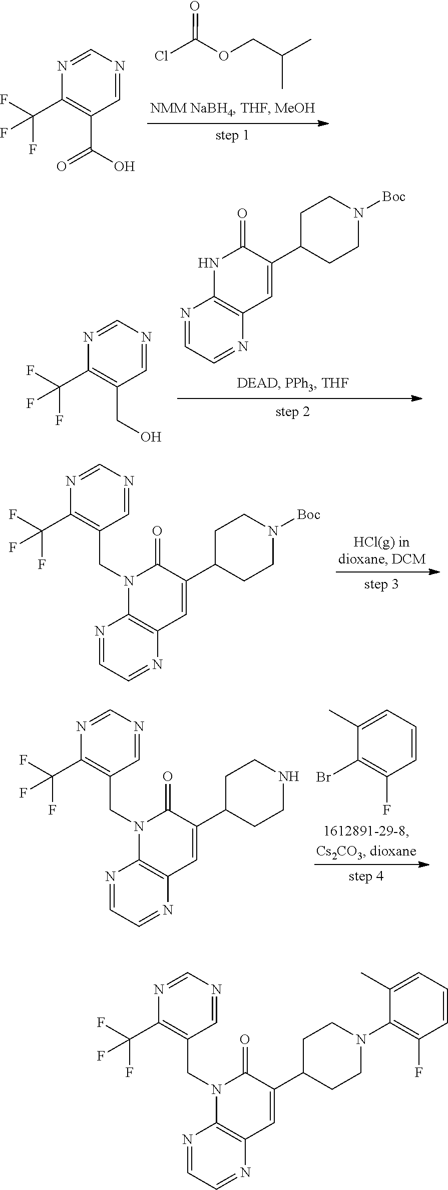

Step 1: (4-(trifluoromethyl)pyrimidin-5-yl)methanol: Followed general procedure AD using 4-(trifluoromethyl)pyrimidine-5-carboxylic acid (100 mg, 0.521 mmol, 1 equiv) as the starting material to give (4-(trifluoromethyl)pyrimidin-5-yl)methanol (35 mg, 37%) as a light yellow oil. MS m/z: 179 [M+H]⁺.

Step 2: tert-butyl 4-(6-oxo-5-((4-(trifluoromethyl)pyrimidin-5-yl)methyl)-5,6-dihydropyrido[2,3-b]pyrazin-7-yl)piperidine-1-carboxylate: Followed general procedure X using tert-butyl 4-(6-oxo-5,6-dihydropyrido[2,3-b]pyrazin-7-yl)piperidine-1-carboxylate (100 mg, 0.303 mmol, 1 equiv) and (4-(trifluoromethyl)pyrimidin-5-yl)methanol (80.9 mg, 0.454 mmol, 1.5 equiv) as the starting materials to give tert-butyl 4-(6-oxo-5-((4-(trifluoromethyl)pyrimidin-5-yl)methyl)-5,6-dihydropyrido[2,3-b]pyrazin-7-yl)piperidine-1-carboxylate (45 mg, 30%) as a yellow solid. MS m/z: 491 [M+H]⁺.

Step 3: 7-(piperidin-4-yl)-5-((4-(trifluoromethyl)pyrimidin-5-yl)methyl)pyrido[2,3-b]pyrazin-6(5H)-one: Followed general procedure F using tert-butyl 4-(6-oxo-5-((4-(trifluoromethyl)pyrimidin-5-yl)methyl)-5,6-dihydropyrido[2,3-b]pyrazin-7-yl)piperidine-1-carboxylate (45 mg, 0.092 mmol, 1 equiv) as the starting material to give the crude product 7-(piperidin-4-yl)-5-((4-(trifluoromethyl)pyrimidin-5-yl)methyl)pyrido[2,3-b]pyrazin-6(5H)-one e (30 mg) as a yellow oil. MS m/z: 391 [M+H]⁺.

Step 4: 7-(1-(2-fluoro-6-methylphenyl)piperidin-4-yl)-5-((4-(trifluoromethyl)pyrimidin-5-yl)methyl)pyrido[2,3-b]pyrazin-6(5H)-one: Followed general procedure G using 7-(piperidin-4-yl)-5-((4-(trifluoromethyl)pyrimidin-5-yl)methyl)pyrido[2,3-b]pyrazin-6(5H)-one (40 mg, 0.102 mmol, 1 equiv) and 2-bromo-1-fluoro-3-methylbenzene (28.8 mg, 0.153 mmol, 1.5 equiv) as the starting materials to give 7-(1-(2-fluoro-6-methylphenyl)piperidin-4-yl)-5-((4-(trifluoromethyl)pyrimidin-5-yl)methyl)pyrido[2,3-b]pyrazin-6(5H)-one (4.4 mg, 11.4%) as an off-white solid. ¹H NMR (400 MHz, Chloroform-d) 69.23 (s, 1H), 8.59-8.48 (m, 1H), 8.43-8.36 (m, 1H), 8.30 (s, 1H), 7.93 (s, 1H), 7.03-6.93 (m, 2H), 6.93-6.85 (m, 1H), 5.95 (s, 2H), 3.41-3.28 (m, 2H), 3.26-3.05 (m, 3H), 2.36 (s, 3H), 2.08-2.00 (m, 2H), 1.87-1.75 (m, 2H). MS m/z: 499.15 [M+H]⁺.

### 8-((3-(Difluoromethoxy)pyridin-2-yl)methyl)-6-(4-(2-fluoro-6-methylphenyl)piperazin-1-yl)-2-methylpyrido[2,3-d]pyrimidin-7(8H)-one (117)

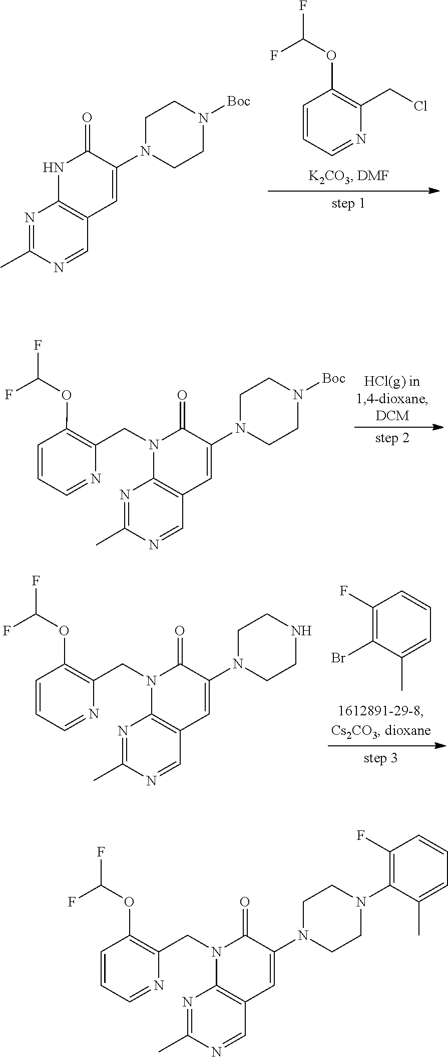

Step 1: tert-butyl 4-(8-((3-(difluoromethoxy)pyridin-2-yl)methyl)-2-methyl-7-oxo-7,8-dihydropyrido[2,3-d]pyrimidin-6-yl)piperazine-1-carboxylate: Followed general procedure E using tert-butyl 4-(2-methyl-7-oxo-7,8-dihydropyrido[2,3-d]pyrimidin-6-yl)piperazine-1-carboxylate (60 mg, 0.17 mmol, 1 equiv) and 2-(chloromethyl)-3-(difluoromethoxy)pyridine (50 mg, 0.261 mmol, 1.5 equiv) as the starting materials to give tert-butyl 4-(8-((3-(difluoromethoxy)pyridin-2-yl)methyl)-2-methyl-7-oxo-7,8-dihydropyrido[2,3-d]pyrimidin-6-yl)piperazine-1-carboxylate (60 mg, 68.7%) as a white solid. MS m/z: 503 [M+H]⁺.

Step 2: 8-((3-(difluoromethoxy)pyridin-2-yl)methyl)-2-methyl-6-(piperazin-1-yl)pyrido[2,3-d]pyrimidin-7(8H)-one: Followed general procedure F using tert-butyl 4-(8-((3-(difluoromethoxy)pyridin-2-yl)methyl)-2-methyl-7-oxo-7,8-dihydropyrido[2,3-d]pyrimidin-6-yl)piperazine-1-carboxylate (60 mg, 0.12 mmol, 1 equiv) as the starting material to give the crude product 8-((3-(difluoromethoxy)pyridin-2-yl)methyl)-2-methyl-6-(piperazin-1-yl)pyrido[2,3-d]pyrimidin-7(8H)-one (40 mg) as a white solid. MS m/z: 403 [M+H]⁺.

Step 3: 8-((3-(difluoromethoxy)pyridin-2-yl)methyl)-6-(4-(2-fluoro-6-methylphenyl)piperazin-1-yl)-2-methylpyrido[2,3-d]pyrimidin-7(8H)-one: Followed general procedure G using 2-methyl-6-(piperazin-1-yl)-8-((3-(trifluoromethyl)pyridin-2-yl)methyl)pyrido[2,3-d]pyrimidin-7(8H)-one (40 mg, 0.1 mmol, 1 equiv) and 2-bromo-1-fluoro-3-methylbenzene (20.7 mg, 0.11 mmol, 1.5 equiv) as the starting materials to give 8-((3-(difluoromethoxy)pyridin-2-yl)methyl)-6-(4-(2-fluoro-6-methylphenyl)piperazin-1-yl)-2-methylpyrido[2,3-d]pyrimidin-7(8H)-one (10.3 mg, 18.7%) as a white solid. ¹H NMR (400 MHz, Chloroform-d) δ 8.76-8.70 (m, 1H), 8.21 (d, J=5.6 Hz, 1H), 7.49 (t, J=7.0 Hz, 1H), 7.19 (s, 1H), 7.06-6.92 (m, 3H), 6.91-6.83 (m, 1H), 6.80-6.64 (m, 1H), 5.92 (d, J=5.8 Hz, 2H), 3.28 (s, 8H), 2.70-2.63 (m, 3H), 2.40-2.34 (m, 3H). MS m/z: 511.5 [M+H]⁺.

### 7-(4-(2-Fluoro-6-methylphenyl)piperazin-1-yl)-5-((3-(trifluoromethyl)pyridin-2-yl)methyl)pyrido[2,3-b]pyrazin-6(5H)-one (118)

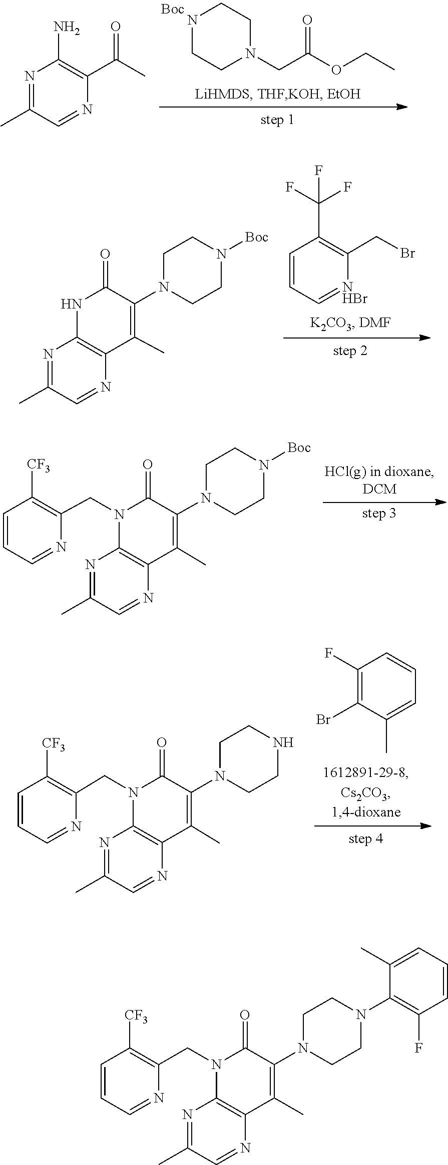

Step 1: tert-butyl 4-(3,8-dimethyl-6-oxo-5,6-dihydropyrido[2,3-b]pyrazin-7-yl)piperazine-1-carboxylate: Followed general procedure D using 1-(3-amino-5-methylpyrazin-2-yl)ethan-1-one (69.4 mg, 0.459 mmol, 1 equiv) and tert-butyl 4-(2-ethoxy-2-oxoethyl)piperazine-1-carboxylate (150 mg, 0.551 mmol, 1.2 equiv) as the starting materials to give tert-butyl 4-(3,8-dimethyl-6-oxo-5,6-dihydropyrido[2,3-b]pyrazin-7-yl)piperazine-1-carboxylate (60 mg, 36%) as a yellow solid. MS m/z: 360 [M+H]⁺.

Step 2: tert-butyl 4-(3,8-dimethyl-6-oxo-5-((3-(trifluoromethyl)pyridin-2-yl)methyl)-5,6-dihydropyrido[2,3-b]pyrazin-7-yl)piperazine-1-carboxylate: Followed general procedure E using tert-butyl 4-(3,8-dimethyl-6-oxo-5,6-dihydropyrido[2,3-b]pyrazin-7-yl)piperazine-1-carboxylate (60 mg, 0.167 mmol, 1 equiv) and 2-(bromomethyl)-3-(trifluoromethyl)pyridine hydrogen bromide (59 mg, 0.184 mmol, 1.1 equiv) as the starting materials to give tert-butyl 4-(3,8-dimethyl-6-oxo-5-((3-(trifluoromethyl)pyridin-2-yl)methyl)-5,6-dihydropyrido[2,3-b]pyrazin-7-yl)piperazine-1-carboxylate (45 mg, 52%) as a yellow solid. MS m/z: 519 [M+H]⁺.

Step 3: 3,8-dimethyl-7-(piperazin-1-yl)-5-((3-(trifluoromethyl)pyridin-2-yl)methyl)pyrido[2,3-b]pyrazin-6(5H)-one: Followed general procedure F using tert-butyl 4-(3,8-dimethyl-6-oxo-5-((3-(trifluoromethyl)pyridin-2-yl)methyl)-5,6-dihydropyrido[2,3-b]pyrazin-7-yl)piperazine-1-carboxylat (45 mg, 0.087 mmol, 1 equiv) as the starting material to give the crude product 3,8-dimethyl-7-(piperazin-1-yl)-5-((3-(trifluoromethyl)pyridin-2-yl)methyl)pyrido[2,3-b]pyrazin-6(5H)-one (30 mg) as a white solid. MS m/z: 419 [M+H]⁺.

Step 4: 7-(4-(2-fluoro-6-methylphenyl)piperazin-1-yl)-3,8-dimethyl-5-((3-(trifluoromethyl)pyridin-2-yl)methyl)pyrido[2,3-b]pyrazin-6(5H)-one: Followed general procedure G using 3,8-dimethyl-7-(piperazin-1-yl)-5-((3-(trifluoromethyl)pyridin-2-yl)methyl)pyrido[2,3-b]pyrazin-6(5H)-one (30 mg, 0.108 mmol, 1 equiv) and 2-bromo-1-fluoro-3-methyl benzene (22 mg, 0.119 mmol, 1.1 equiv) as the starting materials to give 7-(4-(2-fluoro-6-methylphenyl)piperazin-1-yl)-3,8-dimethyl-5-((3-(trifluoromethyl)pyridin-2-yl)methyl)pyrido[2,3-b]pyrazin-6(5H)-one (10.3 mg, 16.8%) as a yellow solid. ¹H NMR (300 MHz, Chloroform-d) δ 8.43 (d, J=4.9 Hz, 1H), 8.32 (s, 1H), 8.00-7.91 (m, 1H), 7.24-7.20 (m, 1H), 7.04-6.94 (m, 2H), 6.94-6.81 (m, 1H), 6.01 (s, 2H), 3.20 (s, 8H), 2.70 (s, 3H), 2.47 (s, 3H), 2.35 (s, 3H). MS m/z: 527.2 [M+H]⁺.

### 6-(4-(2-Fluoro-6-methylphenyl)piperazin-1-yl)-2-methyl-8-((3-(trifluoromethyl)pyridin-2-yl)methyl)pyrido[2,3-d]pyrimidin-7(8H)-one (119)

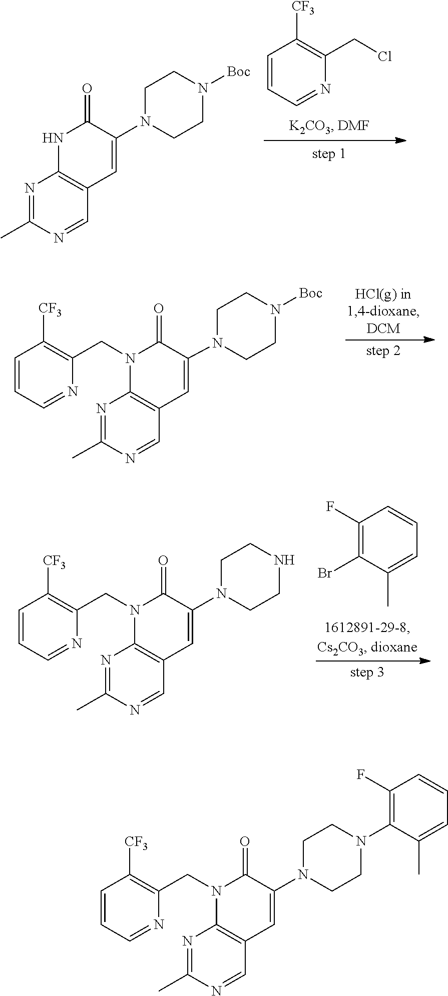

Step 1: tert-butyl 4-(2-methyl-7-oxo-8-((3-(trifluoromethyl)pyridin-2-yl)methyl)-7,8-dihydropyrido[2,3-d]pyrimidin-6-yl)piperazine-1-carboxylate: Followed general procedure E using tert-butyl 4-(2-methyl-7-oxo-7,8-dihydropyrido[2,3-d]pyrimidin-6-yl)piperazine-1-carboxylate (100 mg, 0.29 mmol, 1 equiv) and 2-(chloromethyl)-3-(trifluoromethyl)pyridine (85 mg, 0.435 mmol, 1.5 equiv) as the starting materials to give tert-butyl 4-(2-methyl-7-oxo-8-((3-(trifluoromethyl)pyridin-2-yl)methyl)-7,8-dihydropyrido[2,3-d]pyrimidin-6-yl)piperazine-1-carboxylate (60 mg, 41%) as a white solid. MS m/z: 505 [M+H]⁺.

Step 2: 2-methyl-6-(piperazin-1-yl)-8-((3-(trifluoromethyl)pyridin-2-yl)methyl)pyrido[2,3-d]pyrimidin-7(8H)-one: Followed general procedure F using tert-butyl 4-(3-methyl-6-oxo-5-(2-(trifluoromethoxy)benzyl)-5,6-dihydropyrido[2,3-b]pyrazin-7-yl)piperidine-1-carboxylate (60 mg, 0.12 mmol, 1 equiv) as the starting material to give the crude product 2-methyl-6-(piperazin-1-yl)-8-((3-(trifluoromethyl)pyridin-2-yl)methyl)pyrido[2,3-d]pyrimidin-7(8H)-one (45 mg) as a white solid. MS m/z: 405 [M+H]⁺.

Step 3: 6-(4-(2-fluoro-6-methylphenyl)piperazin-1-yl)-2-methyl-8-((3-(trifluoromethyl)pyridin-2-yl)methyl)pyrido[2,3-d]pyrimidin-7(8H)-one: Followed general procedure G using 2-methyl-6-(piperazin-1-yl)-8-((3-(trifluoromethyl)pyridin-2-yl)methyl)pyrido[2,3-d]pyrimidin-7(8H)-one (45 mg, 0.11 mmol, 1 equiv) and 2-bromo-1-fluoro-3-methylbenzene (31.6 mg, 0.17 mmol, 1.5 equiv) as the starting materials to give 6-(4-(2-fluoro-6-methylphenyl)piperazin-1-yl)-2-methyl-8-((3-(trifluoromethyl)pyridin-2-yl)methyl)pyrido[2,3-d]pyrimidin-7(8H)-one (23.8 mg, 41.1%) as a white solid. 1H NMR (400 MHz, Chloroform-d) δ 8.73 (s, 1H), 8.44 (d, J=4.8 Hz, 1H), 7.97 (d, J=7.8 Hz, 1H), 7.01-6.88 (m, 5H), 6.03 (s, 2H), 3.28 (s, 8H), 2.68 (s, 3H), 2.36 (s, 3H). MS m/z: 513.2 [M+H]⁺.

### 5-((3-(Difluoromethyl)pyrazin-2-yl)methyl)-7-(1-(2-fluoro-6-methylphenyl)piperidin-4-yl)-3-methylpyrido[2,3-b]pyrazin-6(5H)-one (120)

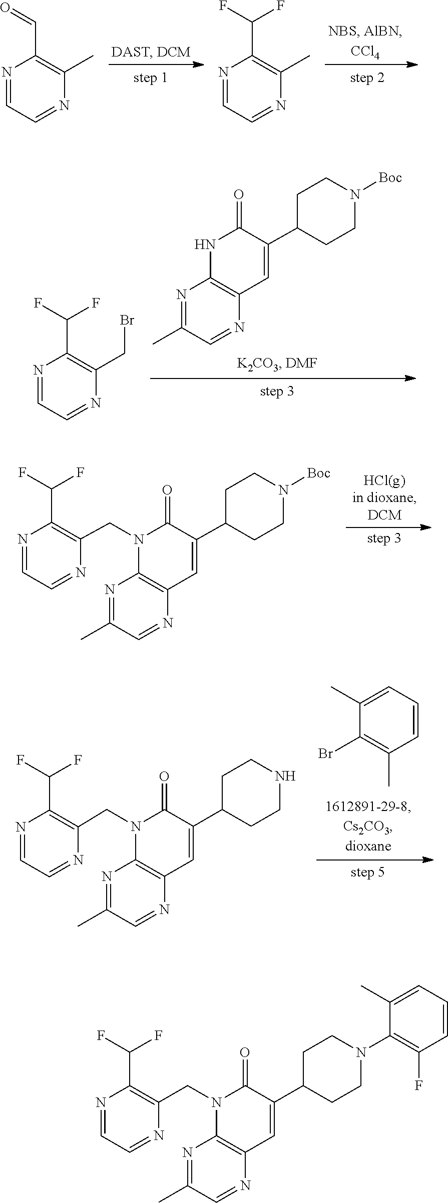

Step 1: 2-(difluoromethyl)-3-methylpyrazine: Followed general procedure AE using 3-methylpyrazine-2-carbaldehyde (200 mg, 1.63 mmol, 1 equiv) and DAST (791 mg, 4.91 mmol, 3 equiv) as the starting materials to give 2-(difluoromethyl)-3-methylpyrazine (60 mg, 25.4%) as a colorless oil. MS m/z: 145 [M+H]⁺.

Step 2: 2-(bromomethyl)-3-(difluoromethyl)pyrazine: Followed general procedure AF using 2-(difluoromethyl)-3-methylpyrazine (100 mg, 0.694 mmol, 1 equiv) as the starting material to give the crude product 2-(bromomethyl)-3-(difluoromethyl)pyrazine used in the next step directly without further purification. MS m/z: 223 [M+H]⁺.

Step 3: tert-butyl 4-(5-((3-(difluoromethyl)pyrazin-2-yl)methyl)-3-methyl-6-oxo-5,6-dihydropyrido[2,3-b]pyrazin-7-yl)piperidine-1-carboxylate: Followed general procedure E using tert-butyl 4-(3-methyl-6-oxo-5,6-dihydropyrido[2,3-b]pyrazin-7-yl)piperidine-1-carboxylate (100 mg, 0.29 mmol, 1 equiv) and 2-(bromomethyl)-3-(difluoromethyl)pyrazine (64.5 mg, 0.29 mmol, 1 equiv) as the starting materials to give tert-butyl 4-(5-((3-(difluoromethyl)pyrazin-2-yl)methyl)-3-methyl-6-oxo-5,6-dihydropyrido[2,3-b]pyrazin-7-yl)piperidine-1-carboxylate (40 mg, 28%) as a yellow solid. MS m/z: 487 [M+H]⁺.

Step 4: 5-((3-(difluoromethyl)pyrazin-2-yl)methyl)-3-methyl-7-(piperidin-4-yl)pyrido[2,3-b]pyrazin-6(5H)-one: Followed general procedure F using tert-butyl 4-(5-((3-(difluoromethyl)pyrazin-2-yl)methyl)-3-methyl-6-oxo-5,6-dihydropyrido[2,3-b]pyrazin-7-yl)piperidine-1-carboxylate (40 mg, 0.082 mmol, 1 equiv) as the starting material to give the crude product 5-((3-(difluoromethyl)pyrazin-2-yl)methyl)-3-methyl-7-(piperidin-4-yl)pyrido[2,3-b]pyrazin-6(5H)-one (30 mg) as a yellow oil. MS m/z: 387 [M+H]⁺.

Step 5: 5-((3-(difluoromethyl)pyrazin-2-yl)methyl)-7-(1-(2-fluoro-6-methylphenyl)piperidin-4-yl)-3-methylpyrido[2,3-b]pyrazin-6(5H)-one: Followed general procedure G using 5-((3-(difluoromethyl)pyrazin-2-yl)methyl)-3-methyl-7-(piperidin-4-yl)pyrido[2,3-b]pyrazin-6(5H)-one (30 mg, 0.077 mmol, 1 equiv) and 2-bromo-1-fluoro-3-methylbenzene (21.9 mg, 0.116 mmol, 1.5 equiv) as the starting materials to give 5-((3-(difluoromethyl)pyrazin-2-yl)methyl)-7-(1-(2-fluoro-6-methylphenyl)piperidin-4-yl)-3-methylpyrido[2,3-b]pyrazin-6(5H)-one (16.1 mg, 41.9%) as a light yellow solid. ¹H NMR (400 MHz, Chloroform-d) 68.47-8.38 (m, 2H), 8.35 (s, 1H), 7.86 (s, 1H), 7.14-6.82 (m, 4H), 6.10 (s, 2H), 3.35 (t, J=11.8 Hz, 2H), 3.22-3.06 (m, 3H), 2.53 (s, 3H), 2.38 (s, 3H), 2.08-1.99 (m, 2H), 1.92-1.80 (m, 2H). MS m/z: 495.35 [M+H]⁺.

### 7-(trans-4-(2-Fluoro-6-methylphenyl)cyclohexyl)-3-methyl-5-((3-(trifluoromethyl)pyridin-2-yl)methyl)pyrido[2,3-b]pyrazin-6(5H)-one (121a); 7-(cis-4-(2-Fluoro-6-methylphenyl)cyclohexyl)-3-methyl-5-((3-(trifluoromethyl)pyridin-2-yl)methyl)pyrido[2,3-b]pyrazin-6(5H)-one (121b)

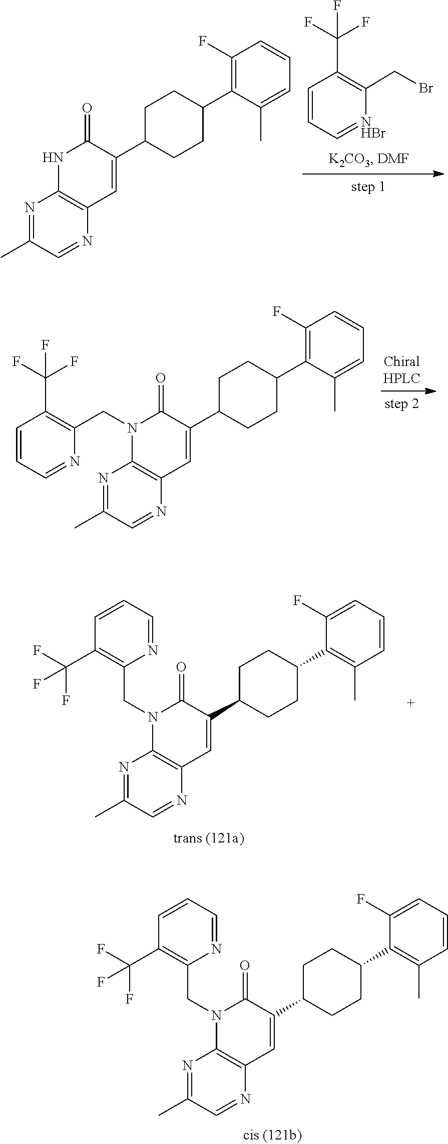

Step 1: 7-(4-(2-fluoro-6-methylphenyl)cyclohexyl)-3-methyl-5-((3-(trifluoromethyl)pyridin-2-yl)methyl)pyrido[2,3-b]pyrazin-6(5H)-one: Followed general procedure D using 7-(4-(2-fluoro-6-methylphenyl)cyclohexyl)-3-methylpyrido[2,3-b]pyrazin-6(5H)-one (80 mg, 0.227 mmol, 1 equiv) and 2-(bromomethyl)-3-(trifluoromethyl)pyridine (80.4 mg, 0.25 mmol, 1.1 equiv) as the starting materials to give the crude product 7-(4-(2-fluoro-6-methylphenyl)cyclohexyl)-3-methyl-5-((3-(trifluoromethyl)pyridin-2-yl)methyl)pyrido[2,3-b]pyrazin-6(5H)-one (60 mg, 51.6%) as a white solid. The crude product was purified by prep-HPLC with the following conditions Column: (Column: Xselect CSH C18 OBD Column 30*150 mm 5 m, n; Mobile Phase A: Water (0.1% FA), Mobile Phase B: ACN; Flow rate: 60 mL/min; Gradient: 70% B to 80% B in 8 min, 80% B; Wave Length: 254/220 nm; RT1 (min): 10.52; Number Of Runs: 0) to afford (R)-7-((1r,4r)-4-(2-fluoro-6-methylphenyl)cyclohexyl)-3-methyl-5-((3-(trifluoromethyl)pyridin-2-yl)methyl)pyrido[2,3-b]pyrazin-6(5H)-one (21 mg, 28.6%) as a white solid and (S)-7-((1s,4s)-4-(2-fluoro-6-methylphenyl)cyclohexyl)-3-methyl-5-((3-(trifluoromethyl)pyridin-2-yl)methyl)pyrido[2,3-b]pyrazin-6(5H)-one (18.4 mg, 20.9%) as a white solid.

7-((1r,4r)-4-(2-Fluoro-6-methylphenyl)cyclohexyl)-3-methyl-5-((3-(trifluoromethyl)pyridin-2-yl)methyl)pyrido[2,3-b]pyrazin-6(5H)-one (121a) ¹H NMR (400 MHz, Chloroform-d) δ 8.44-8.40 (m, 1H), 8.36 (s, 1H), 8.20 (s, 1H), 7.96 (d, J=7.9 Hz, 1H), 7.24-7.20 (m, 1H), 7.06-6.99 (m, 1H), 6.94-6.90 (m, 1H), 6.86-6.80 (m, 1H), 6.03 (s, 2H), 3.14 (t, 1H, J=12.2 Hz), 2.89 (t, 1H, J=12.6 Hz), 2.52 (s, 3H), 2.37 (s, 3H), 2.31-2.16 (m, 4H), 1.99-1.91 (m, 2H), 1.69-1.63 (m, 2H). MS m/z: 511.30 [M+H]⁺

7-((1s,4s)-4-(2-Fluoro-6-methylphenyl)cyclohexyl)-3-methyl-5-((3-(trifluoromethyl)pyridin-2-yl)methyl)pyrido[2,3-b]pyrazin-6(5H)-one (121b)¹H NMR (400 MHz, Chloroform-d) δ 8.45-8.41 (m, 1H), 8.32 (s, 1H), 7.97 (d, J=7.9 Hz, 1H), 7.81 (s, 1H), 7.25-7.22 (m, 1H), 7.06-7.00 (m, 1H), 6.92 (d, J=7.5 Hz, 1H), 6.88-6.81 (m, 1H), 6.03 (s, 2H), 3.48 (t, 1H, J=6 Hz), 2.94 (t, 1H, J=12.3 Hz), 2.52 (s, 3H), 2.38 (s, 3H), 2.19-2.11 (m, 4H), 1.88-1.81 (m, 2H), 1.56-1.48 (m, 2H). MS m/z: 511.3 [M+H]⁺

### 5-((3-(Difluoromethyl)pyrazin-2-yl)methyl)-7-(1-(2-fluoro-6-methylphenyl)piperidin-4-yl)pyrido[2,3-b]pyrazin-6(5H)-one (122)

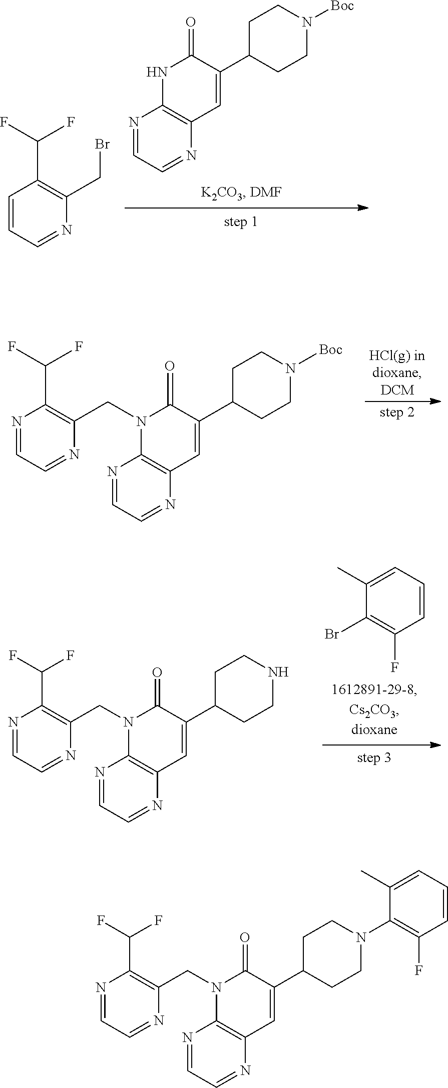

Step 1: tert-butyl 4-(5-((3-(difluoromethyl)pyrazin-2-yl)methyl)-6-oxo-5,6-dihydropyrido[2,3-b]pyrazin-7-yl)piperidine-1-carboxylate: Followed general procedure E using tert-butyl 4-(6-oxo-5,6-dihydropyrido[2,3-b]pyrazin-7-yl)piperidine-1-carboxylate (100 mg, 0.303 mmol, 1 equiv) and 2-(bromomethyl)-3-(difluoromethyl)pyrazine (67.2 mg, 0.303 mmol, 1 equiv) as the starting materials to give tert-butyl 4-(5-((3-(difluoromethyl)pyrazin-2-yl)methyl)-6-oxo-5,6-dihydropyrido[2,3-b]pyrazin-7-yl)piperidine-1-carboxylate (60 mg, 42%) as a yellow solid. MS m/z: 473 [M+H]⁺.

Step 2: 5-((3-(difluoromethyl)pyrazin-2-yl)methyl)-7-(piperidin-4-yl)pyrido[2,3-b]pyrazin-6(5H)-one: Followed general procedure F using tert-butyl 4-(5-((3-(difluoromethyl)pyrazin-2-yl)methyl)-6-oxo-5,6-dihydropyrido[2,3-b]pyrazin-7-yl)piperidine-1-carboxylate (60 mg, 0.127 mmol, 1 equiv) as the starting material to give the crude product 5-((3-(difluoromethyl)pyrazin-2-yl)methyl)-7-(piperidin-4-yl)pyrido[2,3-b]pyrazin-6(5H)-one (50 mg) as a yellow oil. MS m/z: 373 [M+H]⁺.

Step 3: 5-((3-(difluoromethyl)pyrazin-2-yl)methyl)-7-(1-(2-fluoro-6-methylphenyl)piperidin-4-yl)pyrido[2,3-b]pyrazin-6(5H)-one: Followed general procedure G using 5-((3-(difluoromethyl)pyrazin-2-yl)methyl)-7-(piperidin-4-yl)pyrido[2,3-b]pyrazin-6(5H)-one (50 mg, 0.134 mmol, 1 equiv) and 2-bromo-1-fluoro-3-methylbenzene (37.9 mg, 0.201 mmol, 1.5 equiv) as the starting materials to give 5-((3-(difluoromethyl)pyrazin-2-yl)methyl)-7-(1-(2-fluoro-6-methylphenyl)piperidin-4-yl)pyrido[2,3-b]pyrazin-6(5H)-one (21.6 mg, 33.4%) as a light yellow solid. ¹H NMR (400 MHz, Chloroform-d) δ 8.49 (d, J=2.4 Hz, 1H), 8.43 (d, J=2.4 Hz, 1H), 8.39-8.35 (m, 2H), 7.89 (s, 1H), 7.10-6.80 (m, 4H), 6.11 (s, 2H), 3.40-3.27 (m, 2H), 3.21-3.07 (m, 3H), 2.37 (s, 3H), 2.10-2.00 (m, 2H), 1.90-1.77 (m, 2H). MS m/z: 481.15 [M+H]⁺.

### 7-(4-(2-Fluoro-6-methylphenyl)piperazin-1-yl)-5-((3-(trifluoromethoxy)pyridin-2-yl)methyl)pyrido[2,3-b]pyrazin-6(5H)-one (123)

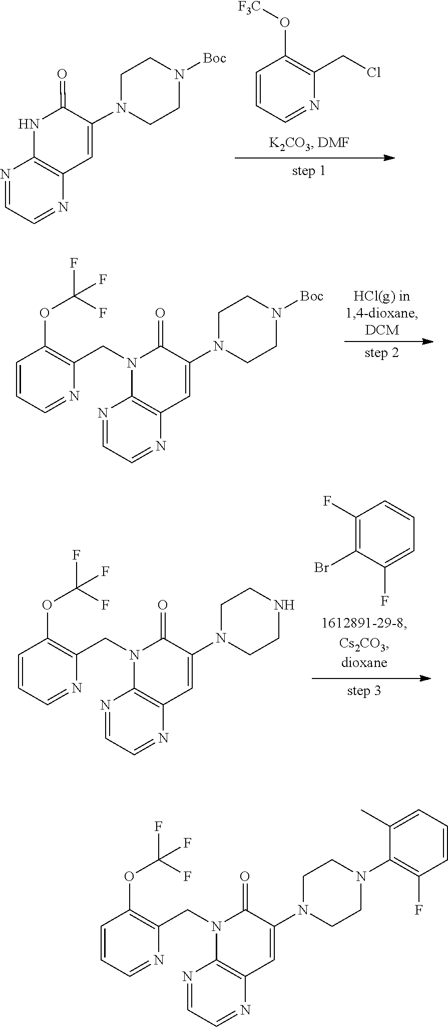 [SMILES: CC(C)(C)OC(=O)N1CCN(c2cc3nccnc3[nH]c2=O)CC1 ; FC(F)(F)Oc1cccnc1CCl ; CC(C)(C)OC(=O)N1CCN(c2cc3nccnc3n(Cc3ncccc3OC(F)(F)F)c2=O)CC1 ; O=c1c(N2CCNCC2)cc2nccnc2n1Cc1ncccc1OC(F)(F)F ; Fc1cccc(F)c1Br ; Cc1cccc(F)c1N1CCN(c2cc3nccnc3n(Cc3ncccc3OC(F)(F)F)c2=O)CC1]

Step 1: tert-butyl 4-(6-oxo-5-((3-(trifluoromethoxy)pyridin-2-yl)methyl)-5,6-dihydropyrido[2,3-b]pyrazin-7-yl)piperazine-1-carboxylate: Followed general procedure E using tert-butyl 4-(6-oxo-5,6-dihydropyrido[2,3-b]pyrazin-7-yl)piperazine-1-carboxylate (90 mg, 0.27 mmol, 1 equiv) and 2-(chloromethyl)-3-(trifluoromethoxy)pyridine (86 mg, 0.408 mmol, 1.5 equiv) as the starting materials to give tert-butyl 4-(6-oxo-5-((3-(trifluoromethoxy)pyridin-2-yl)methyl)-5,6-dihydropyrido[2,3-b]pyrazin-7-yl)piperazine-1-carboxylate (80 mg, 58%) as a white solid. MS m/z: 507 [M+H]⁺.

Step 2: 7-(piperazin-1-yl)-5-((3-(trifluoromethoxy)pyridin-2-yl)methyl)pyrido[2,3-b]pyrazin-6(5H)-one: Followed general procedure F using tert-butyl 4-(8-((3-(difluoromethoxy)pyridin-2-yl)methyl)-2-methyl-7-oxo-7,8-dihydropyrido[2,3-d]pyrimidin-6-yl)piperazine-1-carboxylate (80 mg, 0.158 mmol, 1 equiv) as the starting material to give the crude product 7-(piperazin-1-yl)-5-((3-(trifluoromethoxy)pyridin-2-yl)methyl)pyrido[2,3-b]pyrazin-6(5H)-one (60 mg) as a white solid. MS m/z: 407 [M+H]⁺.

Step 3: 7-(4-(2-fluoro-6-methylphenyl)piperazin-1-yl)-5-((3-(trifluoromethoxy)pyridin-2-yl)methyl)pyrido[2,3-b]pyrazin-6(5H)-one: Followed general procedure G using 7-(piperazin-1-yl)-5-((3-(trifluoromethoxy)pyridin-2-yl)methyl)pyrido[2,3-b]pyrazin-6(5H)-one (60 mg, 0.148 mmol, 1 equiv) and 2-bromo-1-fluoro-3-methylbenzene (41.9 mg, 0.222 mmol, 1.5 equiv) as the starting materials to give 7-(4-(2-fluoro-6-methylphenyl)piperazin-1-yl)-5-((3-(trifluoromethoxy)pyridin-2-yl)methyl)pyrido[2,3-b]pyrazin-6(5H)-one (30.1 mg, 40.8%) as a yellow solid. ¹H NMR (400 MHz, Chloroform-d) δ 8.31 (d, J=2.8 Hz, 1H), 8.25 (d, J=4.7 Hz, 1H), 8.23 (d, J=2.7 Hz, 1H), 7.59 (d, J=8.3 Hz, 1H), 7.34 (s, 2H), 7.22-7.18 (m, 1H), 7.00 (d, J=12.6 Hz, 2H), 6.92-6.82 (m, 1H), 5.95 (s, 2H), 3.28 (s, 4H), 2.37 (s, 4H), 1.25 (s, 3H). MS m/z: 515.2 [M+H]⁺.

### 7-(4-(2-Fluoro-6-methylphenyl)piperazin-1-yl)-3-methyl-5-((3-(trifluoromethoxy)pyridin-2-yl)methyl)pyrido[2,3-b]pyrazin-6(5H)-one (124)

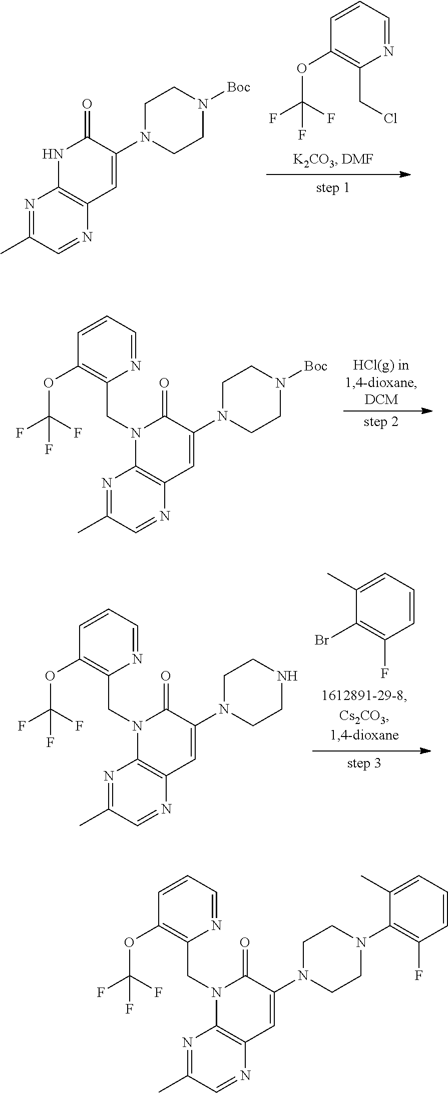

Step 1: tert-butyl 4-(3-methyl-6-oxo-5-((3-(trifluoromethoxy)pyridin-2-yl)methyl)-5,6-dihydropyrido[2,3-b]pyrazin-7-yl)piperazine-1-carboxylate: Followed general procedure E using tert-butyl 4-(3-methyl-6-oxo-5,6-dihydropyrido[2,3-b]pyrazin-7-yl)piperazine-1-carboxylate (150 mg, 0.434 mmol, 1 equiv) and 2-(chloromethyl)-3-(trifluoromethoxy)pyridine (137 mg, 0.651 mmol, 1.5 equiv) as the starting materials to give tert-butyl 4-(3-methyl-6-oxo-5-((3-(trifluoromethoxy)pyridin-2-yl)methyl)-5,6-dihydropyrido[2,3-b]pyrazin-7-yl)piperazine-1-carboxylate (160 mg, 70%) as a yellow oil. MS m/z: 521 [M+H]⁺.

Step 2: 3-methyl-7-(piperazin-1-yl)-5-((3-(trifluoromethoxy)pyridin-2-yl)methyl)pyrido[2,3-b]pyrazin-6(5H)-one: Followed general procedure F using tert-butyl 4-(3-methyl-6-oxo-5-((3-(trifluoromethoxy)pyridin-2-yl)methyl)-5,6-dihydropyrido[2,3-b]pyrazin-7-yl)piperazine-1-carboxylate (160 mg, 0.307 mmol, 1 equiv) as the starting material to give the crude product 3-methyl-7-(piperazin-1-yl)-5-((3-(trifluoromethoxy)pyridin-2-yl)methyl)pyrido[2,3-b]pyrazin-6(5H)-one (100 mg) as a yellow solid. MS m/z: 421 [M+H]⁺.

Step 3: 7-(4-(2-fluoro-6-methylphenyl)piperazin-1-yl)-3-methyl-5-((3-(trifluoromethoxy)pyridin-2-yl)methyl)pyrido[2,3-b]pyrazin-6(5H)-one: Followed general procedure G using 3-methyl-7-(piperazin-1-yl)-5-((3-(trifluoromethoxy)pyridin-2-yl)methyl)pyrido[2,3-b]pyrazin-6(5H)-one (100 mg, 0.238 mmol, 1 equiv) and 2-bromo-1-fluoro-3-methylbenzene (53.9 mg, 0.286 mmol, 1.2 equiv) as the starting materials to give 7-(4-(2-fluoro-6-methylphenyl)piperazin-1-yl)-3-methyl-5-((3-(trifluoromethoxy)pyridin-2-yl)methyl)pyrido[2,3-b]pyrazin-6(5H)-one (88.9 mg, 67.5%) as an orange solid. ¹H NMR (400 MHz, Chloroform-d) δ 8.27-8.26 (m, 2H), 7.59-7.55 (m, 1H), 7.20-7.17 (m, 2H), 7.02-6.95 (m, 2H), 6.89-6.84 (m, 1H), 5.95 (s, 2H), 3.54-3.27 (m, 8H), 2.49 (s, 3H), 2.37 (s, 3H). MS m/z: 529.3 [M+H]⁺.

### 7-(4-(2-Fluoro-6-methylphenyl)piperazin-1-yl)-5-((3-methoxypyrazin-2-yl)methyl)-3-methylpyrido[2,3-b]pyrazin-6(5H)-one (125)

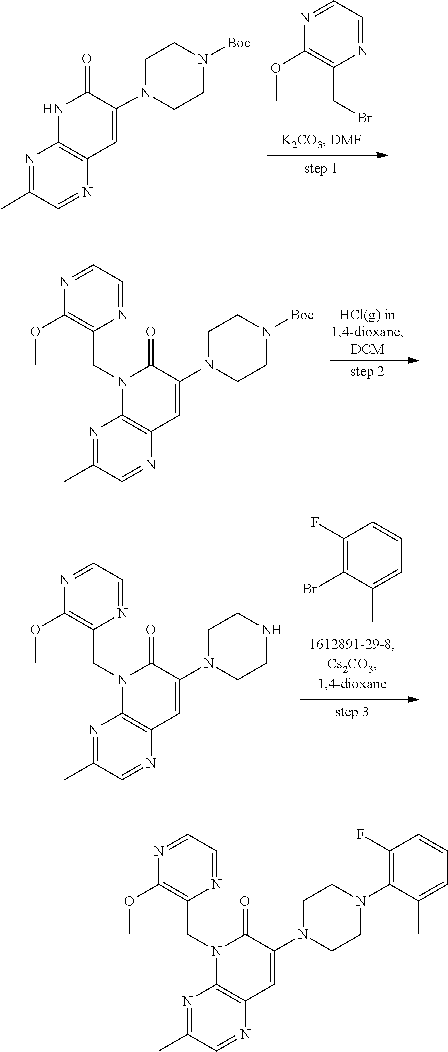

Step 1: tert-butyl 4-(5-((3-methoxypyrazin-2-yl)methyl)-3-methyl-6-oxo-5,6-dihydropyrido[2,3-b]pyrazin-7-yl)piperazine-1-carboxylate: Followed general procedure E using tert-butyl 4-(3-methyl-6-oxo-5,6-dihydropyrido[2,3-b]pyrazin-7-yl)piperazine-1-carboxylate (140 mg, 0.405 mmol, 1 equiv) and 2-(bromomethyl)-3-methoxypyrazine (123 mg, 0.608 mmol, 1.5 equiv) as the starting materials to give tert-butyl 4-(5-((3-methoxypyrazin-2-yl)methyl)-3-methyl-6-oxo-5,6-dihydropyrido[2,3-b]pyrazin-7-yl)piperazine-1-carboxylate (160 mg, 84%) as a yellow oil. MS m/z: 468 [M+H]⁺.

Step 2: 5-((3-methoxypyrazin-2-yl)methyl)-3-methyl-7-(piperazin-1-yl)pyrido[2,3-b]pyrazin-6(5H)-one: Followed general procedure F using tert-butyl 4-(5-((3-methoxypyrazin-2-yl)methyl)-3-methyl-6-oxo-5,6-dihydropyrido[2,3-b]pyrazin-7-yl)piperazine-1-carboxylate (160 mg, 0.342 mmol, 1 equiv) as the starting material to give the crude product 5-((3-methoxypyrazin-2-yl)methyl)-3-methyl-7-(piperazin-1-yl)pyrido[2,3-b]pyrazin-6(5H)-one (130 mg) as a yellow oil. MS m/z: 368 [M+H]⁺.

Step 3: 7-(4-(2-fluoro-6-methylphenyl)piperazin-1-yl)-5-((3-methoxypyrazin-2-yl)methyl)-3-methylpyrido[2,3-b]pyrazin-6(5H)-one: Followed general procedure G using 5-((3-methoxypyrazin-2-yl)methyl)-3-methyl-7-(piperazin-1-yl)pyrido[2,3-b]pyrazin-6(5H)-one (130 mg, 0.354 mmol, 1 equiv) and 2-bromo-1-fluoro-3-methylbenzene (267 mg, 1.41 mmol, 4 equiv) as the starting materials to give 7-(4-(2-fluoro-6-methylphenyl)piperazin-1-yl)-5-((3-methoxypyrazin-2-yl)methyl)-3-methylpyrido[2,3-b]pyrazin-6(5H)-one (68.1 mg, 39%) as a yellow solid. ¹H NMR (400 MHz, Chloroform-d) δ 8.20 (s, 1H), 7.92 (d, J=2.8 Hz, 1H), 7.82 (d, J=2.8 Hz, 1H), 7.32 (s, 1H), 7.03-6.96 (m, 2H), 6.89-6.84 (m, 1H), 5.84 (s, 2H), 4.08 (s, 3H), 3.54-3.27 (m, 8H), 2.51 (s, 3H), 2.36 (s, 3H). MS m/z: 476.2 [M+H]⁺.

### 7-(1-(2-Fluoro-6-(trifluoromethyl)phenyl)piperidin-4-yl)-5-((3-methylpyrazin-2-yl)methyl)pyrido[2,3-b]pyrazin-6(5H)-one (126)

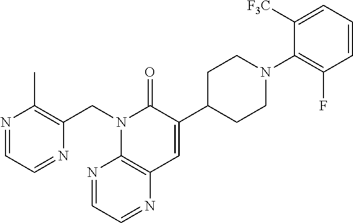 [SMILES: Cc1nccnc1Cn1c(=O)c(C2CCN(c3c(F)cccc3C(F)(F)F)CC2)cc2nccnc21]

Step 1: tert-butyl 4-(5-((3-methylpyrazin-2-yl)methyl)-6-oxo-5,6-dihydropyrido[2,3-b]pyrazin-7-yl)piperidine-1-carboxylate: Followed the General Procedure E using tert-butyl 4-(6-oxo-5,6-dihydropyrido[2,3-b]pyrazin-7-yl)piperidine-1-carboxylate (500 mg, 1.51 mmol, 1.0 equiv.) and 2-(chloromethyl)-3-methylpyrazine hydrochloride (299 mg, 1.66 mmol, 1.1 equiv.) as the starting materials to give tert-butyl 4-(5-((3-methylpyrazin-2-yl)methyl)-6-oxo-5,6-dihydropyrido[2,3-b]pyrazin-7-yl)piperidine-1-carboxylate (450 mg, 68.2%) as a light yellow solid. MS m/z: 437 [M+H]⁺.

Step 2: 5-((3-methylpyrazin-2-yl)methyl)-7-(piperidin-4-yl)pyrido[2,3-b]pyrazin-6(5H)-one: Followed the General Procedure F using tert-butyl 4-(5-((3-methylpyrazin-2-yl)methyl)-6-oxo-5,6-dihydropyrido[2,3-b]pyrazin-7-yl)piperidine-1-carboxylate (450 mg, 1.03 mmol, 1.0 equiv.) as the starting material to give the crude product 5-((3-methylpyrazin-2-yl)methyl)-7-(piperidin-4-yl)pyrido[2,3-b]pyrazin-6(5H)-one (290 mg) as a light yellow solid. MS m/z: 337 [M+H]⁺.

Step 3: 7-(1-(2-fluoro-6-(trifluoromethyl)phenyl)piperidin-4-yl)-5-((3-methylpyrazin-2-yl)methyl)pyrido[2,3-b]pyrazin-6(5H)-one: Followed the General Procedure G using 5-((3-methylpyrazin-2-yl)methyl)-7-(piperidin-4-yl)pyrido[2,3-b]pyrazin-6(5H)-one (290 mg, 0.861 mmol, 1.0 equiv.) and 2-bromo-1-fluoro-3-(trifluoromethyl)benzene (334 mg, 1.29 mmol, 1.5 equiv.) as the starting materials, using BINAP (53.6 mg, 0.086 mmol, 0.1 equiv.) and BINAP Pd G4 (86.6 mg, 0.086 mmol, 0.1 equiv.) as the catalyst to give 7-(1-(2-fluoro-6-(trifluoromethyl)phenyl)piperidin-4-yl)-5-((3-methylpyrazin-2-yl)methyl)pyrido[2,3-b]pyrazin-6(5H)-one (12.1 mg, 2.8%) as a white solid. ¹H NMR (300 MHz, DMSO-d₆) δ 8.60 (d, J=2.4 Hz, 1H), 8.54 (d, J=2.4 Hz, 1H), 8.34 (d, J=2.7 Hz, 1H), 8.15 (d, J=2.7 Hz, 1H), 7.89 (s, 1H), 7.63-7.53 (m, 2H), 7.51-7.43 (m, 1H), 5.78 (s, 2H), 3.22 (t, J=11.3 Hz, 2H), 3.08-2.97 (m, 3H), 2.72 (s, 3H), 1.95 (d, J=12.0 Hz, 2H), 1.83-1.69 (m, 2H). MS m/z: 499.25 [M+H]⁺.

### 7-(1-(2-Fluoro-4-methylpyridin-3-yl)piperidin-4-yl)-5-((3-(trifluoromethyl)pyridin-2-yl)methyl)pyrido[2,3-b]pyrazin-6(5H)-one (127)

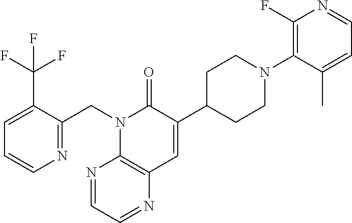 [SMILES: Cc1ccnc(F)c1N1CCC(c2cc3nccnc3n(Cc3ncccc3C(F)(F)F)c2=O)CC1]

Step 1: tert-butyl 4-(6-oxo-5-((3-(trifluoromethyl)pyridin-2-yl)methyl)-5,6-dihydropyrido[2,3-b]pyrazin-7-yl)piperidine-1-carboxylate: Followed the General Procedure E using tert-butyl 4-(6-oxo-5,6-dihydropyrido[2,3-b]pyrazin-7-yl)piperidine-1-carboxylate (200 mg, 0.604 mmol, 1.0 equiv.) and 2-(bromomethyl)-3-(trifluoromethyl)pyridine hydrobromide (213 mg, 0.665 mmol, 1.2 equiv.) as the starting materials to give tert-butyl 4-(6-oxo-5-((3-(trifluoromethyl)pyridin-2-yl)methyl)-5,6-dihydropyrido[2,3-b]pyrazin-7-yl)piperidine-1-carboxylate (200 mg, 67%) as a light yellow solid. MS m/z: 490 [M+H]⁺.

Step 2: 7-(piperidin-4-yl)-5-((3-(trifluoromethyl)pyridin-2-yl)methyl)pyrido[2,3-b]pyrazin-6(5H)-one: Followed the General Procedure F using tert-butyl 4-(6-oxo-5-((3-(trifluoromethyl)pyridin-2-yl)methyl)-5,6-dihydropyrido[2,3-b]pyrazin-7-yl)piperidine-1-carboxylate (200 mg, 0.408 mmol, 1.0 equiv.) as the starting material to give the crude product 7-(piperidin-4-yl)-5-((3-(trifluoromethyl)pyridin-2-yl)methyl)pyrido[2,3-b]pyrazin-6(5H)-one (140 mg) as a yellow solid. MS m/z: 390 [M+H]⁺.

Step 3: 7-(1-(2-fluoro-4-methylpyridin-3-yl)piperidin-4-yl)-5-((3-(trifluoromethyl)pyridin-2-yl)methyl)pyrido[2,3-b]pyrazin-6(5H)-one: Followed the General Procedure G using 7-(piperidin-4-yl)-5-((3-(trifluoromethyl)pyridin-2-yl)methyl)pyrido[2,3-b]pyrazin-6 (5H)-one (140 mg, 0.360 mmol, 1.0 equiv.) and 3-bromo-2-fluoro-4-methylpyridine (103 mg, 0.540 mmol, 1.5 equiv.) as the starting materials to give 7-(1-(2-fluoro-4-methylpyridin-3-yl)piperidin-4-yl)-5-((3-(trifluoromethyl)pyridin-2-yl)methyl)pyrido[2,3-b]pyrazin-6(5H)-one (65.4 mg, 35.7%) as a yellow solid. ¹H NMR (300 MHz, DMSO-d₆) δ 8.60 (d, J=2.4 Hz, 1H), 8.57-8.49 (m, 2H), 8.24 (d, J=7.9 Hz, 1H), 8.00-7.94 (m, 1H), 7.83 (dd, J=4.9, 1.3 Hz, 1H), 7.48 (dd, J=7.9, 4.9 Hz, 1H), 7.20 (d, J=4.9 Hz, 1H), 5.91 (s, 2H), 3.22-2.88 (m, 5H), 2.39 (s, 3H), 2.02-1.71 (m, 4H). MS m/z: 499.25 [M+H]⁺.

### 7-(1-(2-Fluoro-4-methylpyridin-3-yl)piperidin-4-yl)-5-((3-methylpyrazin-2-yl)methyl)pyrido[2,3-b]pyrazin-6(5H)-one (128)

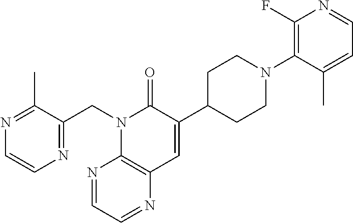 [SMILES: Cc1ccnc(F)c1N1CCC(c2cc3nccnc3n(Cc3nccnc3C)c2=O)CC1]

Step 1: tert-butyl 4-(5-((3-methylpyrazin-2-yl)methyl)-6-oxo-5,6-dihydropyrido[2,3-b]pyrazin-7-yl)piperidine-1-carboxylate: Followed the General Procedure E using tert-butyl 4-(6-oxo-5,6-dihydropyrido[2,3-b]pyrazin-7-yl)piperidine-1-carboxylate (140 mg, 0.424 mmol, 1 equiv) and 2-(chloromethyl)-3-methylpyrazine hydrochloride (90.6 mg, 0.509 mmol, 1.2 equiv) as the starting materials to give tert-butyl 4-(5-((3-methylpyrazin-2-yl)methyl)-6-oxo-5,6-dihydropyrido[2,3-b]pyrazin-7-yl)piperidine-1-carboxylate (120 mg, 65%) as a yellow oil. MS m/z: 437 [M+H]⁺.

Step 2: 5-((3-methylpyrazin-2-yl)methyl)-7-(piperidin-4-yl)pyrido[2,3-b]pyrazin-6(5H)-one: Followed the General Procedure F using tert-butyl 4-(5-((3-methylpyrazin-2-yl)methyl)-6-oxo-5,6-dihydropyrido[2,3-b]pyrazin-7-yl)piperidine-1-carboxylate (120 mg, 0.275 mmol, 1 equiv) as the starting material to give the crude product 5-((3-methylpyrazin-2-yl)methyl)-7-(piperidin-4-yl)pyrido[2,3-b]pyrazin-6(5H)-one (100 mg) as a yellow oil. MS m/z: 337 [M+H]⁺.

Step 3: 7-(1-(2-fluoro-4-methylpyridin-3-yl)piperidin-4-yl)-5-((3-methylpyrazin-2-yl)methyl)pyrido[2,3-b]pyrazin-6(5H)-one: Followed the General Procedure G using 5-((3-methylpyrazin-2-yl)methyl)-7-(piperidin-4-yl)pyrido[2,3-b]pyrazin-6(5H)-one (90 mg, 0.268 mmol, 1 equiv) and 3-bromo-2-fluoro-4-methylpyridine (61.00 mg, 0.322 mmol, 1.2 equiv) as the starting materials to give 7-(1-(2-fluoro-4-methylpyridin-3-yl)piperidin-4-yl)-5-((3-methylpyrazin-2-yl)methyl)pyrido[2,3-b]pyrazin-6(5H)-one (53.2 mg, 41.6%) as an off-white solid. ¹H NMR (400 MHz, Chloroform-d) δ 8.48 (d, J=2.4 Hz, 1H), 8.36 (d, J=2.4 Hz, 1H), 8.30-8.28 (m, 1H), 8.13-8.10 (m, 1H), 7.87 (s, 1H), 7.83-7.80 (m, 1H), 7.01 (d, J=5.0 Hz, 1H), 5.84 (s, 2H), 3.29 (t, J=11.8, 5.8 Hz, 2H), 3.22-3.14 (m, 1H), 3.05 (d, J=11.7 Hz, 2H), 2.80 (s, 3H), 2.39 (s, 3H), 2.13-2.01 (m, 2H), 1.89-1.79 (m, 2H). MS m/z: 446.25 [M+H]⁺.

### 7-(1-(2-Fluoro-4-methylpyridin-3-yl)piperidin-4-yl)-5-((3-(trifluoromethyl)pyrazin-2-yl)methyl)pyrido[2,3-b]pyrazin-6(5H)-one (129)

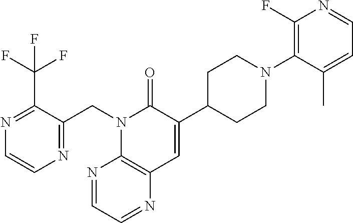 [SMILES: Cc1ccnc(F)c1N1CCC(c2cc3nccnc3n(Cc3nccnc3C(F)(F)F)c2=O)CC1]

Step 1: tert-butyl 4-(6-oxo-5-((3-(trifluoromethyl)pyrazin-2-yl)methyl)-5,6-dihydropyrido[2,3-b]pyrazin-7-yl)piperidine-1-carboxylate: Followed the General Procedure E using tert-butyl 4-(6-oxo-5,6-dihydropyrido[2,3-b]pyrazin-7-yl)piperidine-1-carboxylate (140 mg, 424 μmol, 1 equiv) and 2-(bromomethyl)-3-(trifluoromethyl)pyrazine (122 mg, 509 μmol, 1.2 equiv) as the starting materials to give tert-butyl 4-(6-oxo-5-((3-(trifluoromethyl)pyrazin-2-yl)methyl)-5,6-dihydropyrido[2,3-b]pyrazin-7-yl)piperidine-1-carboxylate (120 mg, 57%) as a white solid. MS m/z: 491 [M+H]⁺.

Step 2: 7-(piperidin-4-yl)-5-((3-(trifluoromethyl)pyrazin-2-yl)methyl)pyrido[2,3-b]pyrazin-6(5H)-one hydrochloride: Followed the General Procedure F using tert-butyl 4-(6-oxo-5-((3-(trifluoromethyl)pyrazin-2-yl)methyl)-5,6-dihydropyrido[2,3-b]pyrazin-7-yl)piperidine-1-carboxylate (120 mg, 245 μmol, 1 equiv) as the starting material to give the crude product 7-(piperidin-4-yl)-5-((3-(trifluoromethyl)pyrazin-2-yl)methyl)pyrido[2,3-b]pyrazin-6(5H)-one hydrochloride (90 mg). MS m/z: 391 [M+H]⁺.

Step 3: 7-(1-(2-fluoro-4-methylpyridin-3-yl)piperidin-4-yl)-5-((3-(trifluoromethyl)pyrazin-2-yl)methyl)pyrido[2,3-b]pyrazin-6(5H)-one: Followed the General Procedure G using 7-(piperidin-4-yl)-5-((3-(trifluoromethyl)pyrazin-2-yl)methyl)pyrido[2,3-b]pyrazin-6(5H)-one (90 mg, 230 μmol, 1 equiv) and 3-bromo-2-fluoro-4-methylpyridine (52 mg, 276 μmol, 1.2 equiv.) as the starting materials to give 7-(1-(2-fluoro-4-methylpyridin-3-yl)piperidin-4-yl)-5-((3-(trifluoromethyl)pyrazin-2-yl)methyl)pyrido[2,3-b]pyrazin-6(5H)-one (36.5 mg, 35%) as a white solid. ¹H NMR (400 MHz, DMSO-d₆) δ 8.75-8.68 (m, 2H), 8.62 (d, J=2.4 Hz, 1H), 8.54 (d, J=2.4 Hz, 1H), 7.98 (d, J=0.9 Hz, 1H), 7.83 (dd, J=4.9, 1.2 Hz, 1H), 7.20 (d, J=4.8 Hz, 1H), 5.97 (s, 2H), 3.16-3.02 (m, 4H), 3.02-2.92 (m, 1H), 2.39 (s, 3H), 1.96-1.89 (m, 2H), 1.89-1.75 (m, 2H). MS m/z: 500.15 [M+H]⁺.

### 7-(1-(2-Fluoro-4-methylpyridin-3-yl)piperidin-4-yl)-3-methyl-5-((3-(trifluoromethyl) pyrazin-2-yl)methyl)pyrido[2,3-b]pyrazin-6(5H)-one (130)

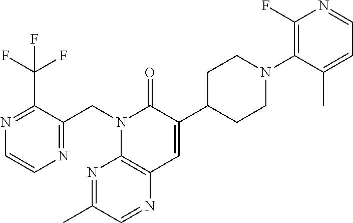 [SMILES: Cc1cnc2cc(C3CCN(c4c(C)ccnc4F)CC3)c(=O)n(Cc3nccnc3C(F)(F)F)c2n1]

Step 1: tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate: Followed the General Procedure E using tert-butyl 4-(3-methyl-6-oxo-5,6-dihydropyrido[2,3-b]pyrazin-7-yl)piperidine-1-carboxylate (150 mg, 0.436 mmol, 1 equiv) and 2-(bromomethyl)-3-(trifluoromethyl)pyrazine (115 mg, 0.480 mmol, 1.2 equiv) as the starting materials to give tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (100 mg, 45%) as a yellow solid. MS m/z: 505 [M+H]⁺.

Step 2: 3-methyl-7-(piperidin-4-yl)-5-((3-(trifluoromethyl)pyrazin-2-yl)methyl)pyrido[2,3-b]pyrazin-6(5H)-one: Followed the General Procedure F using tert-butyl 4-(3-methyl-6-oxo-5-((3-(trifluoromethyl)pyrazin-2-yl)methyl)-5,6-dihydropyrido[2,3-b]pyrazin-7-yl)piperidine-1-carboxylate (100 mg, 0.198 mmol, 1 equiv) as the starting material to give the crude product 3-methyl-7-(piperidin-4-yl)-5-((3-(trifluoromethyl)pyrazin-2-yl)methyl)pyrido[2,3-b]pyrazin-6(5H)-one (80 mg) as a yellow oil. MS m/z: 405 [M+H]⁺.

Step 3: 7-(1-(2-fluoro-4-methylpyridin-3-yl)piperidin-4-yl)-3-methyl-5-((3-(trifluoromethyl) pyrazin-2-yl)methyl)pyrido[2,3-b]pyrazin-6(5H)-one: Followed the General Procedure G using 3-methyl-7-(piperidin-4-yl)-5-((3-(trifluoromethyl)pyrazin-2-yl)methyl)pyrido[2,3-b]pyrazin-6(5H)-one (80 mg, 0.198 mmol, 1 equiv) and 3-bromo-2-fluoro-4-methylpyridine (45.1 mg, 0.238 mmol, 1.2 equiv) as the starting materials to give 7-(1-(2-fluoro-4-methylpyridin-3-yl)piperidin-4-yl)-3-methyl-5-((3-(trifluoromethyl) pyrazin-2-yl)methyl)pyrido[2,3-b]pyrazin-6(5H)-one (41.7 mg, 39.1%) as an off-white solid. ¹H NMR (400 MHz, Chloroform-d) δ 8.51-8.46 (m, 2H), 8.35 (s, 1H), 7.86 (d, J=1.0 Hz, 1H), 7.83-7.81 (m, 1H), 7.01 (d, J=5.0 Hz, 1H), 6.08 (s, 2H), 3.32-3.26 (m, 2H), 3.17-3.11 (m, 1H), 3.05 (d, J=11.8 Hz, 2H), 2.52 (s, 3H), 2.40 (s, 3H), 2.06 (d, J=12.3 Hz, 2H), 1.88-1.78 (m, 2H). MS m/z: 514.3 [M+H]⁺.

### 7-(1-(2-Fluoro-4-methylpyridin-3-yl)piperidin-4-yl)-3-methyl-5-((3-(trifluoromethyl)pyridin-2-yl)methyl)pyrido[2,3-b]pyrazin-6(5H)-one (131)

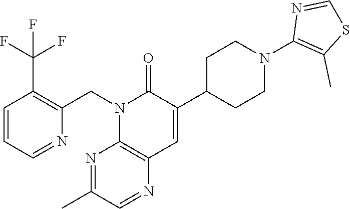 [SMILES: Cc1cnc2cc(C3CCN(c4ncsc4C)CC3)c(=O)n(Cc3ncccc3C(F)(F)F)c2n1]

Step 1: 7-(1-(2-fluoro-4-methylpyridin-3-yl)piperidin-4-yl)-3-methyl-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (60 mg, 0.149 mmol, 1 equiv) and 4-bromo-5-methylthiazole (39.7 mg, 0.223 mmol, 1.5 equiv) as the starting materials to give 7-(1-(2-fluoro-4-methylpyridin-3-yl)piperidin-4-yl)-3-methyl-5-((3-(trifluoromethyl)pyridin-2-yl)methyl)pyrido[2,3-b]pyrazin-6(5H)-one (38.4 mg, 49.8%) as a yellow solid. ¹H NMR (400 MHz, Chloroform-d) δ 8.47-8.40 (m, 2H), 8.33 (s, 1H), 7.97 (d, J=8.0 Hz, 1H), 7.85 (s, 1H), 7.25-7.20 (m, 1H), 6.03 (s, 2H), 3.38 (s, 2H), 3.28-3.01 (m, 3H), 2.51 (s, 3H), 2.40 (d, J=18.4 Hz, 3H), 2.10 (d, J=12.2 Hz, 2H), 1.85 (s, 2H). MS m/z: 501.25 [M+H]⁺

### 7-(1-(1,3,4-Thiadiazol-2-yl)piperidin-4-yl)-3-methyl-5-((3-(trifluoromethyl)pyridin-2-yl)methyl)pyrido[2,3-b]pyrazin-6(5H)-one (132)

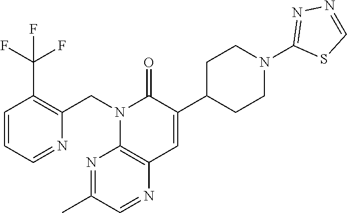 [SMILES: Cc1cnc2cc(C3CCN(c4nncs4)CC3)c(=O)n(Cc3ncccc3C(F)(F)F)c2n1]

Step 1: 7-(1-(1,3,4-thiadiazol-2-yl)piperidin-4-yl)-3-methyl-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (30 mg, 0.074 mmol, 1 equiv) and 2-bromo-1,3,4-thiadiazole (38.4 mg, 0.111 mmol, 1.5 equiv) as the starting materials to give 7-(1-(1,3,4-thiadiazol-2-yl)piperidin-4-yl)-3-methyl-5-((3-(trifluoromethyl)pyridin-2-yl)methyl)pyrido[2,3-b]pyrazin-6(5H)-one (6.3 mg, 16.1%) as a yellow solid. ¹H NMR (400 MHz, Chloroform-d) δ 8.44 (d, J=20.0 Hz, 2H), 8.34 (s, 1H), 7.98 (d, J=8.0 Hz, 1H), 7.77 (s, 1H), 7.24 (s, 2H), 6.03 (s, 2H), 4.15 (d, J=13.0 Hz, 2H), 3.33 (t, J=12.8 Hz, 2H), 2.52 (s, 3H), 2.14 (d, J=13.0 Hz, 2H), 1.79 (d, J=11.0 Hz, 2H). MS m/z: 488.30 [M+H]⁺.

### 7-(1-(2-Fluoro-4-methylpyridin-3-yl)piperidin-4-yl)-3-methyl-5-((3-(trifluoromethyl)pyridin-2-yl)methyl)pyrido[2,3-b]pyrazin-6(5H)-one (133)

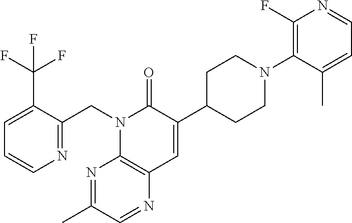 [SMILES: Cc1cnc2cc(C3CCN(c4c(C)ccnc4F)CC3)c(=O)n(Cc3ncccc3C(F)(F)F)c2n1]

Step 1: tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate: Followed the General Procedure E using tert-butyl 4-(3-methyl-6-oxo-5,6-dihydropyrido[2,3-b]pyrazin-7-yl)piperidine-1-carboxylate (50 mg, 0.145 mmol, 1 equiv) and 2-(chloromethyl)-3-(trifluoromethyl)pyridine (31 mg, 0.159 mmol, 1.1 equiv) as the starting materials to give tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (40 mg, 66%) as a white solid. MS m/z: 504 [M+H]⁺.

Step 2: 3-methyl-7-(piperidin-4-yl)-5-((3-(trifluoromethyl)pyridin-2-yl)methyl)pyrido[2,3-b]pyrazin-6(5H)-one: Followed the General Procedure F using tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (40 mg, 0.079 mmol, 1 equiv) as the starting material to give the crude product 3-methyl-7-(piperidin-4-yl)-5-((3-(trifluoromethyl)pyridin-2-yl)methyl)pyrido[2,3-b]pyrazin-6(5H)-one (35 mg) as a white solid. MS m/z: 404 [M+H]⁺.

Step 3: 7-(1-(2-fluoro-4-methylpyridin-3-yl)piperidin-4-yl)-3-methyl-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (35 mg, 0.087 mmol, 1 equiv) and 3-bromo-2-fluoro-4-methylpyridine (24.7 mg, 0.131 mmol, 1.5 equiv) as the starting materials to give 7-(1-(2-fluoro-4-methylpyridin-3-yl)piperidin-4-yl)-3-methyl-5-((3-(trifluoromethyl)pyridin-2-yl)methyl)pyrido[2,3-b]pyrazin-6(5H)-one (5.4 mg, 11.6%) as a yellow solid. ¹H NMR (400 MHz, Chloroform-d) δ 8.46-8.40 (m, 1H), 8.33 (s, 1H), 8.03-7.95 (m, 1H), 7.85 (s, 1H), 7.81 (d, J=4.8 Hz, 1H), 7.24-7.21 (m, 1H), 7.01 (d, J=4.6 Hz, 1H), 6.04 (s, 2H), 3.29 (t, J=11.8 Hz, 2H), 3.16 (s, 1H), 3.04 (d, J=11.4 Hz, 2H), 2.52 (s, 3H), 2.40 (s, 3H), 2.05 (t, J=14.4 Hz, 2H), 1.86-1.79 (m, 2H). MS m/z: 513.15 [M+H]⁺.

### 7-(1-(2-Fluoro-4-methylpyridin-3-yl)piperidin-4-yl)-3-methyl-5-((3-methylpyrazin-2-yl)methyl)pyrido[2,3-b]pyrazin-6(5H)-one (134)

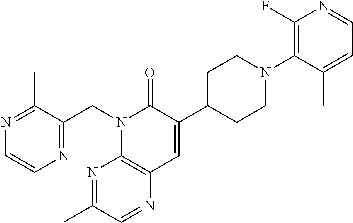 [SMILES: Cc1cnc2cc(C3CCN(c4c(C)ccnc4F)CC3)c(=O)n(Cc3nccnc3C)c2n1]

Step 1: tert-butyl 4-(3-methyl-5-((3-methylpyrazin-2-yl)methyl)-6-oxo-5,6-dihydropyrido[2,3-b]pyrazin-7-yl)piperidine-1-carboxylate: Followed the General Procedure E using tert-butyl 4-(3-methyl-6-oxo-5,6-dihydropyrido[2,3-b]pyrazin-7-yl)piperidine-1-carboxylate (150 mg, 436 μmol, 1.0 equiv) and 2-(chloromethyl)-3-methylpyrazine hydrochloride (93.1 mg, 523 μmol, 1.2 equiv.) as the starting materials to give tert-butyl 4-(3-methyl-5-((3-methylpyrazin-2-yl)methyl)-6-oxo-5,6-dihydropyrido[2,3-b]pyrazin-7-yl)piperidine-1-carboxylate (150 mg, 76%) as a white solid. MS m/z: 451 [M+H]⁺.

Step 2: 3-methyl-5-((3-methylpyrazin-2-yl)methyl)-7-(piperidin-4-yl)pyrido[2,3-b]pyrazin-6(5H)-one: Followed the General Procedure F using tert-butyl 4-(3-methyl-5-((3-methylpyrazin-2-yl)methyl)-6-oxo-5,6-dihydropyrido[2,3-b]pyrazin-7-yl)piperidine-1-carboxylate (150 mg) as the starting material to give the crude product 3-methyl-5-((3-methylpyrazin-2-yl)methyl)-7-(piperidin-4-yl)pyrido[2,3-b]pyrazin-6(5H)-one (110 mg). MS m/z: 351 [M+H]⁺.

Step 3: 7-(1-(2-fluoro-4-methylpyridin-3-yl)piperidin-4-yl)-3-methyl-5-((3-methylpyrazin-2-yl)methyl)pyrido[2,3-b]pyrazin-6(5H)-one: Followed the General Procedure G using 3-methyl-5-((3-methylpyrazin-2-yl)methyl)-7-(piperidin-4-yl)pyrido[2,3-b]pyrazin-6(5H)-one (110 mg, 314 μmol, 1.0 equiv) and 3-bromo-2-fluoro-4-methylpyridine (65.3 mg, 345 μmol, 1.1 equiv) as the starting materials to give 7-(1-(2-fluoro-4-methylpyridin-3-yl)piperidin-4-yl)-3-methyl-5-((3-methylpyrazin-2-yl)methyl)pyrido[2,3-b]pyrazin-6(5H)-one (10.4 mg, 7.2%) as a white solid. ¹H NMR (400 MHz, Chloroform-d) δ 8.35 (s, 1H), 8.30 (s, 1H), 8.21 (s, 1H), 7.83 (d, J=17.3 Hz, 2H), 7.01 (d, J=4.8 Hz, 1H), 5.86 (s, 2H), 3.29 (t, J=11.8 Hz, 2H), 3.15 (t, J=12.0 Hz, 1H), 3.05 (d, J=11.7 Hz, 2H), 2.86 (s, 3H), 2.54 (s, 3H), 2.40 (s, 3H), 2.06 (d, J=12.3 Hz, 2H), 1.81 (d, J=3.8 Hz, 2H). MS m/z: 460.3 [M+H]⁺.

### 7-((1s,4s)-4-(2-Fluoro-4-methylpyridin-3-yl)cyclohexyl)-3-methyl-5-((3-methylpyrazin-2-yl)methyl)pyrido[2,3-b]pyrazin-6(5H)-one (135A); 7-((1r,4r)-4-(2-Fluoro-4-methylpyridin-3-yl)cyclohexyl)-3-methyl-5-((3-methylpyrazin-2-yl)methyl)pyrido[2,3-b]pyrazin-6(5H)-one (135B)

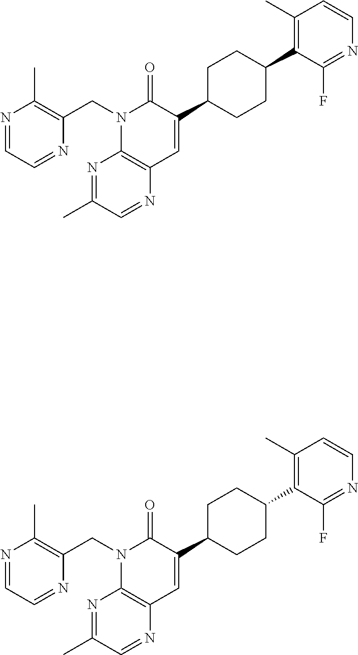 [SMILES: Cc1cnc2cc([C@H]3CC[C@@H](c4c(C)ccnc4F)CC3)c(=O)n(Cc3nccnc3C)c2n1 ; Cc1cnc2cc([C@H]3CC[C@H](c4c(C)ccnc4F)CC3)c(=O)n(Cc3nccnc3C)c2n1]

Step 1: ethyl 2-(4-(2-fluoro-4-methylpyridin-3-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (500 mg, 1.69 mmol, 1.0 equiv.) and 3-bromo-2-fluoro-4-methylpyridine (483 mg, 2.54 mmol, 1.5 equiv.) as the starting materials to give ethyl 2-(4-(2-fluoro-4-methylpyridin-3-yl)cyclohex-3-en-1-yl)acetate (430 mg, 91%) as a light yellow oil. MS m/z: 278 [M+H]⁺.

Step 2: ethyl 2-(4-(2-fluoro-4-methylpyridin-3-yl)cyclohexyl)acetate: Followed the General Procedure AR using ethyl 2-(4-(2-fluoro-4-methylpyridin-3-yl)cyclohex-3-en-1-yl)acetate (430 mg, 1.55 mmol, 1.0 equiv.) as the starting material to give the crude product ethyl 2-(4-(2-fluoro-4-methylpyridin-3-yl)cyclohexyl)acetate (380 mg) as a light yellow oil. MS m/z: 280 [M+H]⁺.

Step 3: 7-(4-(2-fluoro-4-methylpyridin-3-yl)cyclohexyl)-3-methylpyrido[2,3-b]pyrazin-6(5H)-one: Followed the General Procedure D using ethyl 2-(4-(2-fluoro-4-methylpyridin-3-yl)cyclohexyl)acetate (380 mg, 1.36 mmol, 1.0 equiv.) and 3-amino-5-methylpyrazine-2-carbaldehyde (225 mg, 1.63 mmol, 1.2 equiv.) as the starting material to give the product 7-(4-(2-fluoro-4-methylpyridin-3-yl)cyclohexyl)-3-methylpyrido[2,3-b]pyrazin-6(5H)-one (300 mg, 62%) as a light yellow solid. MS m/z: 353 [M+H]⁺.

Step 4: 7-(4-(2-fluoro-4-methylpyridin-3-yl)cyclohexyl)-3-methyl-5-((3-methylpyrazin-2-yl)methyl)pyrido[2,3-b]pyrazin-6(5H)-one: Followed the General Procedure E using 7-(4-(2-fluoro-4-methylpyridin-3-yl)cyclohexyl)-3-methylpyrido[2,3-b]pyrazin-6(5H)-one (150 mg, 0.425 mmol, 1.0 equiv.) and 2-(chloromethyl)-3-methylpyrazine hydrochloride (84.1 mg, 0.467 mmol, 1.1 equiv.) as the starting materials to give the crude product 7-(4-(2-fluoro-4-methylpyridin-3-yl)cyclohexyl)-3-methyl-5-((3-methylpyrazin-2-yl)methyl)pyrido[2,3-b]pyrazin-6(5H)-one (110 mg). The crude product was purified by Prep-HPLC with the following conditions (Column: Xselect CSH C18 OBD Column 30*150 mm 5 m, n; Mobile Phase A: Water (0.1% FA), Mobile Phase B: ACN; Flow rate: 60 mL/min; Gradient: 35% B to 45% B in 11 min; Wave Length: 220 nm; RT1 (min): 11.55); Gradient: isocratic % B) to afford 7-((1s,4s)-4-(2-fluoro-4-methylpyridin-3-yl)cyclohexyl)-3-methyl-5-((3-methylpyrazin-2-yl)methyl)pyrido[2,3-b]pyrazin-6(5H)-one (30.2 mg, 28.1%) as a yellow solid and 7-((1r,4r)-4-(2-fluoro-4-methylpyridin-3-yl)cyclohexyl)-3-methyl-5-((3-methylpyrazin-2-yl)methyl)pyrido[2,3-b]pyrazin-6(5H)-one (34.7 mg, 31.7%) as a yellow solid.

135A: 7-((1s,4s)-4-(2-fluoro-4-methylpyridin-3-yl)cyclohexyl)-3-methyl-5-((3-methylpyrazin-2-yl)methyl)pyrido[2,3-b]pyrazin-6(5H)-one. ¹H NMR (300 MHz, Chloroform-d) δ 8.37 (s, 1H), 8.27 (d, J=2.6 Hz, 1H), 8.14 (dd, J=6.7, 1.8 Hz, 2H), 7.88 (dd, J=5.0, 1.0 Hz, 1H), 6.95 (dd, J=5.0, 0.9 Hz, 1H), 5.84 (s, 2H), 3.46 (d, J=6.7 Hz, 1H), 3.00-2.88 (m, 1H), 2.81 (s, 3H), 2.54 (s, 3H), 2.40 (s, 3H), 2.33 (d, J=13.9 Hz, 2H), 2.18 (q, J=13.2 Hz, 2H), 1.98 (d, J=14.2 Hz, 2H), 1.61-1.58 (m, 2H). MS m/z: 459.15 [M+H]⁺.

135B: 7-((1r,4r)-4-(2-fluoro-4-methylpyridin-3-yl)cyclohexyl)-3-methyl-5-((3-methylpyrazin-2-yl)methyl)pyrido[2,3-b]pyrazin-6(5H)-one. ¹H NMR (300 MHz, Chloroform-d) δ 8.34 (s, 1H), 8.28 (d, J=2.6 Hz, 1H), 8.14 (d, J=2.5 Hz, 1H), 7.93-7.87 (m, 1H), 7.80 (d, J=0.9 Hz, 1H), 6.98-6.91 (m, 1H), 5.84 (s, 2H), 3.13 (t, J=12.0 Hz, 1H), 2.91 (t, J=12.3 Hz, 1H), 2.82 (s, 3H), 2.54 (s, 3H), 2.41 (s, 3H), 2.16 (d, J=12.0 Hz, 4H), 1.85 (s, 1H), 1.81 (s, 1H), 1.58 (t, J=12.6 Hz, 2H). MS m/z: 459.15 [M+H]⁺.

### 3-(1-(2-Fluoro-6-methylphenyl)piperidin-4-yl)-1-((3-(trifluoromethyl)pyrazin-2-yl)methyl)quinoxalin-2(1H)-one (136)

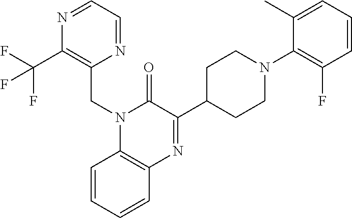 [SMILES: Cc1cccc(F)c1N1CCC(c2nc3ccccc3n(Cc3nccnc3C(F)(F)F)c2=O)CC1]

Step 1: benzyl 4-(3-oxo-3,4-dihydroquinoxalin-2-yl)piperidine-1-carboxylate: Followed the General Procedure P using benzyl 4-(2-methoxy-2-oxoacetyl)piperidine-1-carboxylate (600 mg, 1.96 mmol, 1 equiv.) and 4-methylbenzene-1,2-diamine (319 mg, 2.95 mmol, 1.5 equiv.) as the starting materials to give benzyl 4-(3-oxo-3,4-dihydroquinoxalin-2-yl)piperidine-1-carboxylate (300 mg, 42%) as a yellow solid. MS m/z: 364 [M+H]⁺.

Step 2: tert-butyl 4-(3-oxo-3,4-dihydroquinoxalin-2-yl)piperidine-1-carboxylate: Followed the General Procedure AS using benzyl 4-(6-methyl-3-oxo-3,4-dihydroquinoxalin-2-yl)piperidine-1-carboxylate (300 mg, 0.826 mmol, 1 equiv.) and Boc₂O (180 mg, 0.826 mmol, 1 equiv.) as the starting materials to give tert-butyl 4-(3-oxo-3,4-dihydroquinoxalin-2-yl)piperidine-1-carboxylate (200 mg, 73%) as an off-white solid. MS m/z: 330 [M+H]⁺.

Step 3: tert-butyl 4-(3-oxo-4-((3-(trifluoromethyl)pyrazin-2-yl)methyl)-3,4-dihydroquinoxalin-2-yl)piperidine-1-carboxylate: Followed the General Procedure E using tert-butyl 4-(3-oxo-3,4-dihydroquinoxalin-2-yl)piperidine-1-carboxylate (80 mg 0.243 mmol, 1 equiv.) and 2-(bromomethyl)-3-(trifluoromethyl)pyrazine (87.2 mg, 0.365 mmol, 1.5 equiv.) as the starting materials to give tert-butyl 4-(3-oxo-4-((3-(trifluoromethyl)pyrazin-2-yl)methyl)-3,4-dihydroquinoxalin-2-yl)piperidine-1-carboxylate (60 mg, 50%) as a white solid. MS m/z: 490 [M+H]⁺.

Step 4: 3-(piperidin-4-yl)-1-((3-(trifluoromethyl)pyrazin-2-yl)methyl)quinoxalin-2(1H)-one: Followed the General Procedure F using tert-butyl 4-(3-oxo-4-((3-(trifluoromethyl)pyrazin-2-yl)methyl)-3,4-dihydroquinoxalin-2-yl)piperidine-1-carboxylate (60 mg, 0.123 mmol, 1 equiv.) as the starting material to give the crude product 3-(piperidin-4-yl)-1-((3-(trifluoromethyl)pyrazin-2-yl)methyl)quinoxalin-2(1H)-one (50 mg) as an off-white solid. MS m/z: 390 [M+H]⁺.

Step 5: 3-(1-(2-fluoro-6-methylphenyl)piperidin-4-yl)-1-((3-(trifluoromethyl)pyrazin-2-yl)methyl)quinoxalin-2(1H)-one: Followed the General Procedure G using 3-(piperidin-4-yl)-1-((3-(trifluoromethyl)pyrazin-2-yl)methyl)quinoxalin-2(1H)-one (50 mg, 0.129 mmol, 1.0 equiv.) and 2-bromo-1-fluoro-3-methylbenzene (36.2 mg, 0.193 mmol, 1.5 equiv.) as the starting materials to give 3-(1-(2-fluoro-6-methylphenyl)piperidin-4-yl)-1-((3-(trifluoromethyl)pyrazin-2-yl)methyl)quinoxalin-2(1H)-one (18.7 mg, 17%) as a white solid. ¹H NMR (400 MHz, Chloroform-d) δ 8.75 (d, J=2.4 Hz, 1H), 8.66 (d, J=2.4 Hz, 1H), 8.02 (dd, J=8.0, 1.8 Hz, 1H), 7.73 (dd, J=8.0, 1.8 Hz, 1H), 7.62-7.51 (m, 2H), 7.02-6.94 (m, 2H), 6.91-6.83 (m, 1H), 5.91 (s, 2H), 3.44-3.25 (m, 3H), 3.19-3.06 (m, 2H), 2.39 (s, 3H), 2.23-2.02 (m, 4H). MS m/z: 498.15 [M+H]⁺.

### (S)-7-((1s,4s)-4-(2-Fluoro-6-methylphenyl)cyclohexyl)-3-methyl-5-((3-methylpyrazin-2-yl)methyl)pyrido[2,3-b]pyrazin-6(5H)-one (137A); (R)-7-((1r,4r)-4-(2-Fluoro-6-methylphenyl)cyclohexyl)-3-methyl-5-((3-methylpyrazin-2-yl)methyl)pyrido[2,3-b]pyrazin-6(5H)-one (137B)

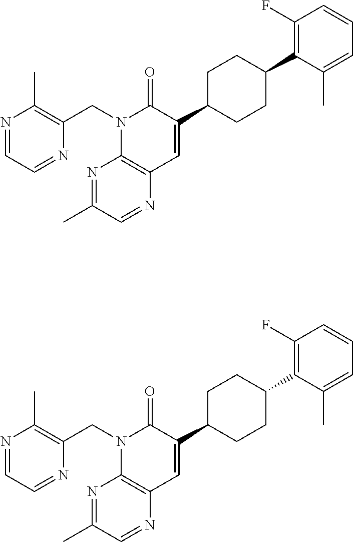 [SMILES: Cc1cnc2cc([C@H]3CC[C@@H](c4c(C)cccc4F)CC3)c(=O)n(Cc3nccnc3C)c2n1 ; Cc1cnc2cc([C@H]3CC[C@H](c4c(C)cccc4F)CC3)c(=O)n(Cc3nccnc3C)c2n1]

Step 1: 7-(4-(2-fluoro-6-methylphenyl)cyclohexyl)-3-methyl-5-((3-(trifluoromethyl)pyridin-2-yl)methyl)pyrido[2,3-b]pyrazin-6(5H)-one: Followed the General Procedure E using 7-(4-(2-fluoro-6-methylphenyl)cyclohexyl)-3-methylpyrido[2,3-b]pyrazin-6(5H)-one (150 mg, 0.427 mmol, 1.0 equiv) and 2-(chloromethyl)-3-methylpyrazine hydrogen chloride (91.7 mg, 0.512 mmol, 1.2 equiv) as the starting materials to give the crude product 7-(4-(2-fluoro-6-methylphenyl)cyclohexyl)-3-methyl-5-((3-methylpyrazin-2-yl)methyl)pyrido[2,3-b]pyrazin-6(5H)-one (130 mg) as a yellow solid. The crude product was purified by Prep-HPLC with the following conditions Column: (Column: Xselect CSH C18 OBD Column 30*150 mm 5 m, n; Mobile Phase A: Water (0.1% FA), Mobile Phase B: ACN; Flow rate: 60 mL/min; Gradient: 55% B to 60% B in 10 min; Wave Length: 220 nm; RT1 (min): 9.85) to afford (S)-7-((1s,4s)-4-(2-fluoro-6-methylphenyl)cyclohexyl)-3-methyl-5-((3-methylpyrazin-2-yl)methyl)pyrido[2,3-b]pyrazin-6(5H)-one (10.9 mg, 11.8%) as a white solid and (R)-7-((1r,4r)-4-(2-fluoro-6-methylphenyl)cyclohexyl)-3-methyl-5-((3-methylpyrazin-2-yl)methyl)pyrido[2,3-b]pyrazin-6(5H)-one (27.4 mg, 29.8%) as a white solid.

137A: (S)-7-((1s,4s)-4-(2-fluoro-6-methylphenyl)cyclohexyl)-3-methyl-5-((3-methylpyrazin-2-yl)methyl)pyrido[2,3-b]pyrazin-6(5H)-one. ¹H NMR (400 MHz, Chloroform-d) δ 8.37 (s, 1H), 8.27 (d, J=2.6 Hz, 1H), 8.21-8.19 (m, 1H), 8.12 (d, J=2.5 Hz, 1H), 7.06-6.99 (m, 1H), 6.92 (d, J=7.5 Hz, 1H), 6.85-6.79 (m, 1H), 5.83 (s, 2H), 3.51-3.44 (m, 1H), 2.99-2.89 (m, 1H), 2.80 (s, 3H), 2.54 (s, 3H), 2.37 (s, 3H), 2.31-2.18 (m, 4H), 2.02-1.91 (m, 2H), 1.69-1.66 (m, 2H). MS m/z: 458.15 [M+H]⁺

137B: (R)-7-((1r,4r)-4-(2-fluoro-6-methylphenyl)cyclohexyl)-3-methyl-5-((3-methylpyrazin-2-yl)methyl)pyrido[2,3-b]pyrazin-6(5H)-one. ¹H NMR (400 MHz, Chloroform-d) δ 8.34 (s, 1H), 8.28 (s, 1H), 8.13 (s, 1H), 7.81 (s, 1H), 7.07-6.99 (m, 1H), 6.92 (d, J=7.5 Hz, 1H), 6.88-6.82 (m, 1H), 5.84 (s, 2H), 3.18-3.09 (m, 1H), 2.96-2.86 (m, 1H), 2.80 (s, 3H), 2.54 (s, 3H), 2.38 (s, 3H), 2.23-2.11 (m, 4H), 1.90-1.81 (m, 2H), 1.56-1.47 (m, 2H). MS m/z: 458.15 [M+H]⁺.

### 5-((3-(Difluoromethyl)pyridin-2-yl)methyl)-7-(1-(2-fluoro-6-methylphenyl)piperidin-4-yl)-3-methylpyrido[2,3-b]pyrazin-6(5H)-one (138)

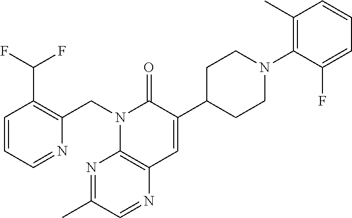 [SMILES: Cc1cnc2cc(C3CCN(c4c(C)cccc4F)CC3)c(=O)n(Cc3ncccc3C(F)F)c2n1]

Step 1: tert-butyl 4-(5-((3-(difluoromethyl)pyridin-2-yl)methyl)-3-methyl-6-oxo-5,6-dihydropyrido[2,3-b]pyrazin-7-yl)piperidine-1-carboxylate: Followed the General Procedure E using tert-butyl 4-(3-methyl-6-oxo-5,6-dihydropyrido[2,3-b]pyrazin-7-yl)piperidine-1-carboxylate (166 mg, 0.482 mmol, 1 equiv) and 2-(bromomethyl)-3-(difluoromethyl)pyridine (160 mg, 0.724 mmol, 1.5 equiv) as the starting materials to give tert-butyl 4-(5-((3-(difluoromethyl)pyridin-2-yl)methyl)-3-methyl-6-oxo-5,6-dihydropyrido[2,3-b]pyrazin-7-yl)piperidine-1-carboxylate (100 mg, 42.7%) as a yellow oil. MS m/z: 486 [M+H]⁺.

Step 2: 5-((3-(difluoromethyl)pyridin-2-yl)methyl)-3-methyl-7-(piperidin-4-yl)pyrido[2,3-b]pyrazin-6(5H)-one: Followed the General Procedure F using tert-butyl 4-(5-((3-(difluoromethyl)pyridin-2-yl)methyl)-3-methyl-6-oxo-5,6-dihydropyrido[2,3-b]pyrazin-7-yl)piperidine-1-carboxylate (100 mg, 0.206 mmol, 1 equiv) as the starting material to give the crude product 5-((3-(difluoromethyl)pyridin-2-yl)methyl)-3-methyl-7-(piperidin-4-yl)pyrido[2,3-b]pyrazin-6(5H)-one (60 mg) as a yellow oil. MS m/z: 386 [M+H]⁺.

Step 3: 5-((3-(difluoromethyl)pyridin-2-yl)methyl)-7-(1-(2-fluoro-6-methylphenyl)piperidin-4-yl)-3-methylpyrido[2,3-b]pyrazin-6(5H)-one: Followed the General Procedure G using 5-((3-(difluoromethyl)pyridin-2-yl)methyl)-3-methyl-7-(piperidin-4-yl)pyrido[2,3-b]pyrazin-6(5H)-one (60 mg, 0.156 mmol, 1 equiv) and 2-bromo-1-fluoro-3-methylbenzene (35.3 mg, 0.187 mmol, 1.2 equiv) as the starting materials to give 5-((3-(difluoromethyl)pyridin-2-yl)methyl)-7-(1-(2-fluoro-6-methylphenyl)piperidin-4-yl)-3-methylpyrido[2,3-b]pyrazin-6(5H)-one (25 mg, 31.5%) as a light yellow solid. ¹H NMR (400 MHz, Chloroform-d) δ 8.44 (d, J=4.7 Hz, 1H), 8.35 (s, 1H), 7.91 (d, J=7.8 Hz, 1H), 7.83 (d, J=1.0 Hz, 1H), 7.36 (d, J=55.5 Hz, 1H), 7.25-7.21 (m, 1H), 7.00-6.96 (m, 2H), 6.89-6.83 (m, 1H), 5.87 (s, 2H), 3.33 (t, J=11.8 Hz, 2H), 3.17-3.07 (m, 3H), 2.56 (s, 3H), 2.36 (s, 3H), 2.01 (d, J=12.3 Hz, 2H), 1.80 (d, J=12.6 Hz, 2H). MS m/z: 494.1 [M+H]⁺.

### 3-(1-(2-Fluoro-6-methylphenyl)piperidin-4-yl)-1-((3-(trifluoromethyl)pyridin-2-yl)methyl)quinoxalin-2(1H)-one (139)

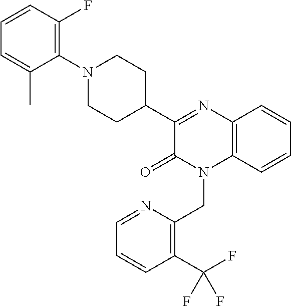 [SMILES: Cc1cccc(F)c1N1CCC(c2nc3ccccc3n(Cc3ncccc3C(F)(F)F)c2=O)CC1]

Step 1: tert-butyl 4-(3-oxo-4-((3-(trifluoromethyl)pyridin-2-yl)methyl)-3,4-dihydroquinoxalin-2-yl)piperidine-1-carboxylate: Followed the General Procedure E using tert-butyl 4-(3-oxo-3,4-dihydroquinoxalin-2-yl)piperidine-1-carboxylate (100 mg, 0.304 mmol, 1 equiv.) and 2-(bromomethyl)-3-(trifluoromethyl)pyridine hydrochloride (100 mg, 0.365 mmol, 1.2 equiv.) as the starting materials to give tert-butyl 4-(3-oxo-4-((3-(trifluoromethyl)pyradin-2-yl)methyl)-3,4-dihydroquinoxalin-2-yl)piperidine-1-carboxylate (70 mg, 47%) as a brown solid. MS m/z: 489 [M+H]⁺.

Step 2: 3-(piperidin-4-yl)-1-((3-(trifluoromethyl)pyridin-2-yl)methyl)quinoxalin-2(1H)-one: Followed the General Procedure F using tert-butyl 4-(3-oxo-4-((3-(trifluoromethyl)pyradin-2-yl)methyl)-3,4-dihydroquinoxalin-2-yl)piperidine-1-carboxylate (70 mg, 0.143 mmol, 1 equiv.) as the starting material to give the crude product 7-methyl-3-(piperidin-4-yl)-1-((3-(trifluoromethyl)pyridin-2-yl)methyl)quinoxalin-2(1H)-one (55 mg, crude) as an off-white solid. MS m/z: 389 [M+H]⁺.

Step 3: 3-(1-(2-fluoro-6-methylphenyl)piperidin-4-yl)-1-((3-(trifluoromethyl)pyridin-2-yl)methyl)quinoxalin-2(1H)-one: Followed the General Procedure G using 7-methyl-3-(piperidin-4-yl)-1-((3-(trifluoromethyl)pyradin-2-yl)methyl)quinoxalin-2(1H)-one (55 mg, 0.142 mmol, 1.0 equiv.) and 2-bromo-1-fluoro-3-methylbenzene (40 mg, 0.213 mmol, 1.5 equiv.) as the starting materials to give 3-(1-(2-fluoro-6-methylphenyl)piperidin-4-yl)-1-((3-(trifluoromethyl)pyradin-2-yl)methyl)quinoxalin-2(1H)-one (21.6 mg, 17.8%) as a white solid. ¹H NMR (300 MHz, Chloroform-d) δ 8.78 (d, J=4.8 Hz, 1H), 8.09-7.95 (m, 2H), 7.80-7.74 (m, 1H), 7.64-7.50 (m, 2H), 7.43 (dd, J=8.1, 4.8 Hz, 1H), 7.01-6.94 (m, 2H), 6.91-6.81 (m, 1H), 5.84 (d, J=1.2 Hz, 2H), 3.43-3.22 (m, 3H), 3.18-3.05 (m, 2H), 2.37 (s, 3H), 2.22-2.02 (m, 4H). MS m/z: 498.15 [M+H]⁺.

### 5-((3-(Difluoromethyl)pyridin-2-yl)methyl)-7-(4-(2-fluoro-6-methylphenyl)piperazin-1-yl)-3-methylpyrido[2,3-b]pyrazin-6(5H)-one (140)

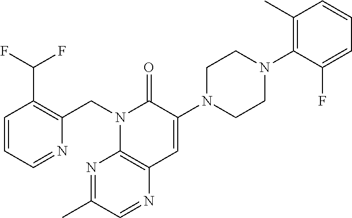 [SMILES: Cc1cnc2cc(N3CCN(c4c(C)cccc4F)CC3)c(=O)n(Cc3ncccc3C(F)F)c2n1]

Step 1: tert-butyl 4-(5-((3-(difluoromethyl)pyridin-2-yl)methyl)-3-methyl-6-oxo-5,6-dihydropyrido[2,3-b]pyrazin-7-yl)piperazine-1-carboxylate: Followed the General Procedure E using tert-butyl 4-(3-methyl-6-oxo-5,6-dihydropyrido[2,3-b]pyrazin-7-yl)piperazine-1-carboxylate (180 mg, 0.521 mmol, 1 equiv) and 2-(bromomethyl)-3-(difluoromethyl)pyridine hydrobromide (127 mg, 0.573 mmol, 1.1 equiv) as the starting materials to give tert-butyl 4-(5-((3-(difluoromethyl)pyridin-2-yl)methyl)-3-methyl-6-oxo-5,6-dihydropyrido[2,3-b]pyrazin-7-yl)piperazine-1-carboxylate (146 mg, 57%) as a light yellow solid. MS m/z: 487 [M+H]⁺.

Step 2: 5-((3-(difluoromethyl)pyridin-2-yl)methyl)-3-methyl-7-(piperazin-1-yl)pyrido[2,3-b]pyrazin-6(5H)-one: Followed the General Procedure F using tert-butyl 4-(5-((3-(difluoromethyl)pyridin-2-yl)methyl)-3-methyl-6-oxo-5,6-dihydropyrido[2,3-b]pyrazin-7-yl)piperazine-1-carboxylate (146 mg, 0.3 mmol, 1 equiv) as the starting material to give the crude product 5-((3-(difluoromethyl)pyridin-2-yl)methyl)-3-methyl-7-(piperazin-1-yl)pyrido[2,3-b]pyrazin-6(5H)-one (114 mg) as a yellow oil. MS m/z: 387 [M+H]⁺.

Step 3: 5-((3-(difluoromethyl)pyridin-2-yl)methyl)-7-(4-(2-fluoro-6-methylphenyl)piperazin-1-yl)-3-methylpyrido[2,3-b]pyrazin-6(5H)-one: Followed the General Procedure G using 5-((3-(difluoromethyl)pyridin-2-yl)methyl)-3-methyl-7-(piperazin-1-yl)pyrido[2,3-b]pyrazin-6(5H)-one (57 mg, 0.148 mmol, 1 equiv) and 2-bromo-1-fluoro-3-methylbenzene (41.8 mg, 0.222 mmol, 1.5 equiv) as the starting materials to give 5-((3-(difluoromethyl)pyridin-2-yl)methyl)-7-(4-(2-fluoro-6-methylphenyl)piperazin-1-yl)-3-methylpyrido[2,3-b]pyrazin-6(5H)-one (34.1 mg, 46.4%) as a yellow solid. ¹H NMR (400 MHz, Chloroform-d) δ 8.44 (d, J=4.9 Hz, 1H), 8.27 (s, 1H), 7.89 (d, J=7.8 Hz, 1H), 7.29 (s, 1H), 7.23-7.20 (m, 2H), 7.02-6.95 (m, 2H), 6.89-6.84 (m, 1H), 5.88 (s, 2H), 3.95-2.85 (m, 8H), 2.52 (s, 3H), 2.36 (s, 3H). MS m/z: 495.10 [M+H]⁺.

### 5-((3-(Difluoromethyl)pyridin-2-yl)methyl)-7-(1-(2-fluoro-4-methylpyridin-3-yl)piperidin-4-yl)-3-methylpyrido[2,3-b]pyrazin-6(5H)-one (141)

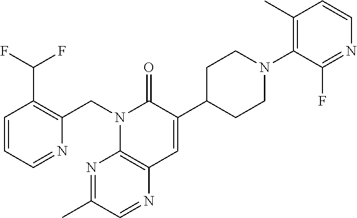 [SMILES: Cc1cnc2cc(C3CCN(c4c(C)ccnc4F)CC3)c(=O)n(Cc3ncccc3C(F)F)c2n1]

Step 1: 5-((3-(difluoromethyl)pyridin-2-yl)methyl)-7-(1-(2-fluoro-4-methylpyridin-3-yl)piperidin-4-yl)-3-methylpyrido[2,3-b]pyrazin-6(5H)-one: Followed the General Procedure G using 5-((3-(difluoromethyl)pyridin-2-yl)methyl)-3-methyl-7-(piperidin-4-yl)pyrido[2,3-b]pyrazin-6(5H)-one (50 mg, 0.130 mmol, 1 equiv) and 3-bromo-2-fluoro-4-methylpyridine (37.0 mg, 0.195 mmol, 1.5 equiv) as the starting materials to give 5-((3-(difluoromethyl)pyridin-2-yl)methyl)-7-(1-(2-fluoro-4-methylpyridin-3-yl)piperidin-4-yl)-3-methylpyrido[2,3-b]pyrazin-6(5H)-one (11.9 mg, 18.3%) as a white solid. ¹H NMR (300 MHz, Chloroform-d) δ 8.43 (s, 1H), 8.34 (d, J=3.6 Hz, 1H), 7.90 (d, J=7.8 Hz, 1H), 7.92 (s, 1H), 7.81 (s, 2H), 7.09 (s, 1H), 7.00 (s, 1H), 5.88 (s, 2H), 3.25 (d, J=12.6 Hz, 2H), 3.17-2.99 (m, 3H), 2.60-2.52 (m, 3H), 2.41-2.36 (m, 3H), 2.04 (d, J=11.8 Hz, 2H), 1.77 (d, J=12.4 Hz, 2H). MS m/z: 495.10 [M+H]⁺.

### 3-(1-(2-Fluoro-6-methylphenyl)piperidin-4-yl)-7-methyl-1-((3-(trifluoromethyl)pyridin-2-yl)methyl)quinoxalin-2(1H)-one (142A); 3-(1-(2-Fluoro-6-methylphenyl)piperidin-4-yl)-6-methyl-1-((3-(trifluoromethyl)pyridin-2-yl)methyl)quinoxalin-2(1H)-one (142B)

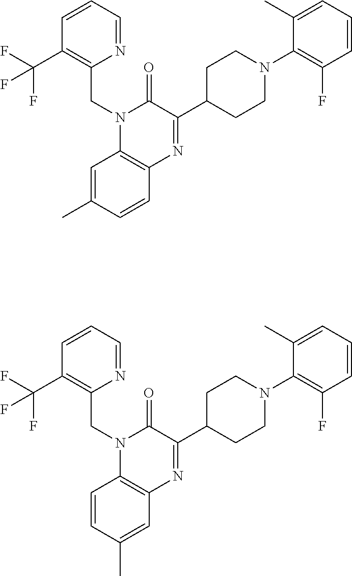 [SMILES: Cc1ccc2nc(C3CCN(c4c(C)cccc4F)CC3)c(=O)n(Cc3ncccc3C(F)(F)F)c2c1 ; Cc1ccc2c(c1)nc(C1CCN(c3c(C)cccc3F)CC1)c(=O)n2Cc1ncccc1C(F)(F)F]

Step 1: tert-butyl 4-(6-methyl-3-oxo-4-((3-(trifluoromethyl)pyradin-2-yl)methyl)-3,4-dihydroquinoxalin-2-yl)piperidine-1-carboxylate: Followed the General Procedure E using tert-butyl 4-(6-methyl-3-oxo-3,4-dihydroquinoxalin-2-yl)piperidine-1-carboxylate (130 mg, 0.379 mmol, 1 equiv.) and 2-(bromomethyl)-3-(trifluoromethyl)pyridine hydrochloride (125 mg, 0.455 mmol, 1.2 equiv.) as the starting materials to give tert-butyl 4-(6-methyl-3-oxo-4-((3-(trifluoromethyl)pyradin-2-yl)methyl)-3,4-dihydroquinoxalin-2-yl)piperidine-1-carboxylate (120 mg, 62.8%) as a brown solid. MS m/z: 503 [M+H]⁺.

Step 2: 7-methyl-3-(piperidin-4-yl)-1-((3-(trifluoromethyl)pyridin-2-yl)methyl)quinoxalin-2(1H)-one: Followed the General Procedure F using tert-butyl 4-(6-methyl-3-oxo-4-((3-(trifluoromethyl)pyradin-2-yl)methyl)-3,4-dihydroquinoxalin-2-yl)piperidine-1-carboxylate (120 mg, 0.239 mmol, 1 equiv.) as the starting material to give the crude product 7-methyl-3-(piperidin-4-yl)-1-((3-(trifluoromethyl)pyridin-2-yl)methyl)quinoxalin-2(1H)-one (100 mg) as an off-white solid. MS m/z: 403 [M+H]⁺.

Step 3: 3-(1-(2-fluoro-6-methylphenyl)piperidin-4-yl)-7-methyl-1-((3-(trifluoromethyl)pyridin-2-yl)methyl)quinoxalin-2(1H)-one and 3-(1-(2-fluoro-6-methylphenyl)piperidin-4-yl)-6-methyl-1-((3-(trifluoromethyl)pyridin-2-yl)methyl)quinoxalin-2(1H)-one: Followed the General Procedure G using 7-methyl-3-(piperidin-4-yl)-1-((3-(trifluoromethyl)pyradin-2-yl)methyl)quinoxalin-2(1H)-one (100 mg, 0.249 mmol, 1.0 equiv.) and 2-bromo-1-fluoro-3-methylbenzene (70.1 mg, 0.373 mmol, 1.5 equiv.) as the starting materials to give 3-(1-(2-fluoro-6-methylphenyl)piperidin-4-yl)-7-methyl-1-((3-(trifluoromethyl)pyradin-2-yl)methyl)quinoxalin-2(1H)-one (24.1 mg, 18.9%) as a white solid and 3-(1-(2-fluoro-6-methylphenyl)piperidin-4-yl)-6-methyl-1-((3-(trifluoromethyl)pyradin-2-yl)methyl)quinoxalin-2(1H)-one (22.6 mg, 17.8%) as a white solid.

142A: 3-(1-(2-fluoro-6-methylphenyl)piperidin-4-yl)-7-methyl-1-((3-(trifluoromethyl)pyridin-2-yl)methyl)quinoxalin-2(1H)-one: ¹H NMR (400 MHz, Chloroform-d) δ 8.79 (s, 1H), 8.06 (d, J=8.0 Hz, 1H), 7.81 (s, 1H), 7.67 (d, J=8.4 Hz, 1H), 7.48-7.36 (m, 2H), 7.01-6.91 (m, 2H), 6.89-6.80 (m, 1H), 5.82 (s, 2H), 3.39-3.19 (m, 3H), 3.13-3.03 (m, 2H), 2.53 (s, 3H), 2.34 (s, 3H), 2.17-1.95 (m, 4H). MS m/z: 511.25 [M+H]⁺.

142B: 3-(1-(2-fluoro-6-methylphenyl)piperidin-4-yl)-6-methyl-1-((3-(trifluoromethyl)pyridin-2-yl)methyl)quinoxalin-2(1H)-one: ¹H NMR (400 MHz, Chloroform-d) δ 8.78 (d, 1H), 8.06 (d, 1H), 7.89 (d, J=8.4 Hz, 1H), 7.56 (s, 1H), 7.46-7.33 (m, 2H), 7.00-6.92 (m, 2H), 6.90-6.80 (m, 1H), 5.82 (s, 2H), 3.37-3.19 (m, 3H), 3.12-3.02 (m, 2H), 2.52 (s, 3H), 2.34 (s, 3H), 2.16-1.96 (m, 4H). MS m/z: 511.25 [M+H]⁺.

### 7-((1r,4r)-4-(2-Fluoro-4-methylpyridin-3-yl)cyclohexyl)-3-methyl-5-((3-(trifluoromethyl)pyrazin-2-yl)methyl)pyrido[2,3-b]pyrazin-6(5H)-one (143A): 7-((1s,4s)-4-(2-Fluoro-4-methylpyridin-3-yl)cyclohexyl)-3-methyl-5-((3-(trifluoromethyl)pyrazin-2-yl)methyl)pyrido[2,3-b]pyrazin-6(5H)-one (143B)

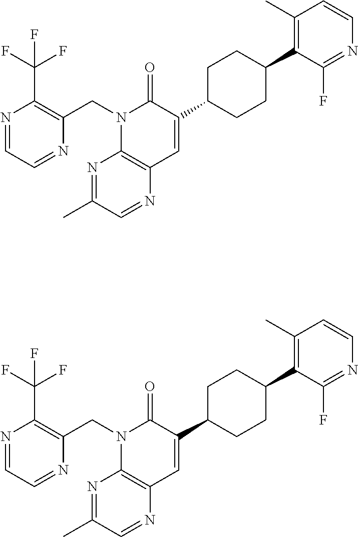 [SMILES: Cc1cnc2cc([C@H]3CC[C@H](c4c(C)ccnc4F)CC3)c(=O)n(Cc3nccnc3C(F)(F)F)c2n1 ; Cc1cnc2cc([C@H]3CC[C@@H](c4c(C)ccnc4F)CC3)c(=O)n(Cc3nccnc3C(F)(F)F)c2n1]

Step 1: 7-(4-(2-fluoro-4-methylpyridin-3-yl)cyclohexyl)-3-methyl-5-((3-(trifluoromethyl)pyrazin-2-yl)methyl)pyrido[2,3-b]pyrazin-6(5H)-one: Followed the General Procedure E using 7-(4-(2-fluoro-4-methylpyridin-3-yl)cyclohexyl)-3-methylpyrido[2,3-b]pyrazin-6(5H)-one (100 mg, 0.284 mmol, 1.0 equiv.) and 2-(bromomethyl)-3-(trifluoromethyl)pyrazine hydrobromide (75.2 mg, 0.312 mmol, 1.1 equiv.) as the starting materials to give the crude product 7-(4-(2-fluoro-4-methylpyridin-3-yl)cyclohexyl)-3-methyl-5-((3-(trifluoromethyl)pyrazin-2-yl)methyl)pyrido[2,3-b]pyrazin-6(5H)-one (120 mg). The crude product was continue separated by Prep-HPLC with the following conditions (Column: Xselect CSH C18 OBD Column 30*150 mm, 5 m; Mobile Phase A: Water (0.05% TFA), Mobile Phase B: ACN; Flow rate: 60 mL/min mL/min; Gradient: 56% B to 68% B in 8 min; Wave Length: 254 nm/220 nm nm; RT1 (min): 6.90/7.35) to afford 7-((1r,4r)-4-(2-fluoro-4-methylpyridin-3-yl)cyclohexyl)-3-methyl-5-((3-(trifluoromethyl)pyrazin-2-yl)methyl)pyrido[2,3-b]pyrazin-6(5H)-one (10.7 mg, 6.89%) as a white solid and 7-((1s,4s)-4-(2-fluoro-4-methylpyridin-3-yl)cyclohexyl)-3-methyl-5-((3-(trifluoromethyl)pyrazin-2-yl)methyl)pyrido[2,3-b]pyrazin-6(5H)-one (23.2 mg, 15.3%) as a white solid.

143A: 7-((1r,4r)-4-(2-fluoro-4-methylpyridin-3-yl)cyclohexyl)-3-methyl-5-((3-(trifluoromethyl)pyrazin-2-yl)methyl)pyrido[2,3-b]pyrazin-6(5H)-one: ¹H NMR (300 MHz, Chloroform-d) δ 8.57-8.45 (m, 2H), 8.37 (s, 1H), 7.96-7.88 (m, 1H), 7.83 (d, J=0.9 Hz, 1H), 6.97 (d, J=4.9 Hz, 1H), 6.10 (s, 2H), 3.14 (t, J=12.1 Hz, 1H), 3.01-2.87 (m, 1H), 2.54 (s, 3H), 2.43 (s, 3H), 2.18 (d, J=11.9 Hz, 4H), 1.91-1.80 (m, 2H), 1.65 (s, 1H), 1.56 (s, 1H). MS m/z: 513.15 [M+H]⁺.

143B: 7-((1s,4s)-4-(2-fluoro-4-methylpyridin-3-yl)cyclohexyl)-3-methyl-5-((3-(trifluoromethyl)pyrazin-2-yl)methyl)pyrido[2,3-b]pyrazin-6(5H)-one: ¹H NMR (300 MHz, Chloroform-d) δ 8.57-8.45 (m, 2H), 8.37 (s, 1H), 7.96-7.88 (m, 1H), 7.83 (d, J=0.9 Hz, 1H), 6.97 (d, J=4.9 Hz, 1H), 6.10 (s, 2H), 3.45 (t, J=12.1 Hz, 1H), 3.01-2.87 (m, 1H), 2.54 (s, 3H), 2.43 (s, 3H), 2.18 (d, J=11.9 Hz, 4H), 1.91-1.80 (m, 2H), 1.65 (s, 1H), 1.56 (s, 1H). MS m/z: 513.15 [M+H]⁺.

### 3-(1-(2-Fluoro-6-methylphenyl)piperidin-4-yl)-7-methyl-1-((3-(trifluoromethyl)pyrazin-2-yl)methyl)quinoxalin-2(1H)-one (144A); 3-(1-(2-Fluoro-6-methylphenyl)piperidin-4-yl)-6-methyl-1-((3-(trifluoromethyl)pyrazin-2-yl)methyl)quinoxalin-2(1H)-one (144B)

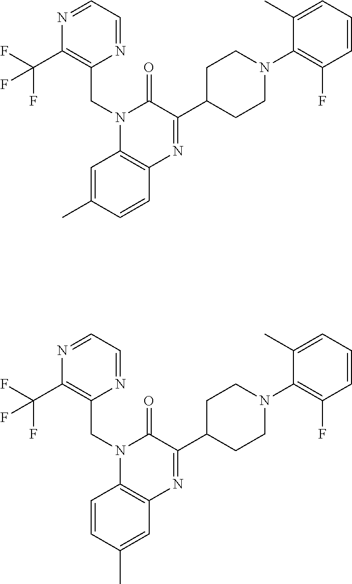 [SMILES: Cc1ccc2nc(C3CCN(c4c(C)cccc4F)CC3)c(=O)n(Cc3nccnc3C(F)(F)F)c2c1 ; Cc1ccc2c(c1)nc(C1CCN(c3c(C)cccc3F)CC1)c(=O)n2Cc1nccnc1C(F)(F)F]

Step 1: benzyl 4-(1-formamido-2-methoxy-2-oxoethylidene)piperidine-1-carboxylate: Followed the General Procedure N using methyl 2-isocyanoacetate (2.76 g, 27.9 mmol, 1.3 equiv.) and benzyl 4-oxopiperidine-1-carboxylate (5 g, 21.5 mmol, 1 equiv.) as the starting materials to give benzyl 4-(1-formamido-2-methoxy-2-oxoethylidene)piperidine-1-carboxylate (1 g, 20.6%) as an off-white solid. MS m/z: 333 [M+H]⁺.

Step 2: benzyl 4-(2-methoxy-2-oxoacetyl)piperidine-1-carboxylate: Followed the General Procedure O using benzyl 4-(1-formamido-2-methoxy-2-oxoethylidene)piperidine-1-carboxylate (1 g, 3.01 mmol, 1 equiv.) as the starting material to give the crude product benzyl 4-(2-methoxy-2-oxoacetyl)piperidine-1-carboxylate (1 g) as a brown oil. MS m/z: 306 [M+H]⁺.

Step 3: benzyl 4-(6-methyl-3-oxo-3,4-dihydroquinoxalin-2-yl)piperidine-1-carboxylate: Followed the General Procedure P using benzyl 4-(2-methoxy-2-oxoacetyl)piperidine-1-carboxylate (1 g, 3.27 mmol, 1 equiv.) and 4-methylbenzene-1,2-diamine (600 mg, 4.92 mmol, 1.5 equiv.) as the starting materials to give benzyl 4-(6-methyl-3-oxo-3,4-dihydroquinoxalin-2-yl)piperidine-1-carboxylate (400 mg, 32%) as a yellow solid. MS m/z: 378 [M+H]⁺.

Step 4: tert-butyl 4-(6-methyl-3-oxo-3,4-dihydroquinoxalin-2-yl)piperidine-1-carboxylate: Followed the General Procedure AS using benzyl 4-(6-methyl-3-oxo-3,4-dihydroquinoxalin-2-yl)piperidine-1-carboxylate (400 mg, 1.06 mmol, 1 equiv.) and Boc₂O (231 mg, 1.06 mmol, 1 equiv.) as the starting materials to give tert-butyl 4-(6-methyl-3-oxo-3,4-dihydroquinoxalin-2-yl)piperidine-1-carboxylate (250 mg, 68%) as an off-white solid. MS m/z: 344 [M+H]⁺.

Step 5: tert-butyl 4-(6-methyl-3-oxo-4-((3-(trifluoromethyl)pyrazin-2-yl)methyl)-3,4-dihydroquinoxalin-2-yl)piperidine-1-carboxylate: Followed the General Procedure E using tert-butyl 4-(6-methyl-3-oxo-3,4-dihydroquinoxalin-2-yl)piperidine-1-carboxylate (120 mg, 0.35 mmol, 1 equiv.) and 2-(bromomethyl)-3-(trifluoromethyl)pyrazine (126 mg, 0.525 mmol, 1.5 equiv.) as the starting materials to give tert-butyl 4-(6-methyl-3-oxo-4-((3-(trifluoromethyl)pyrazin-2-yl)methyl)-3,4-dihydroquinoxalin-2-yl)piperidine-1-carboxylate (100 mg, 56%) as a brown solid. MS m/z: 504 [M+H]⁺.

Step 6: 7-methyl-3-(piperidin-4-yl)-1-((3-(trifluoromethyl)pyrazin-2-yl)methyl)quinoxalin-2(1H)-one: Followed the General Procedure F using tert-butyl 4-(6-methyl-3-oxo-4-((3-(trifluoromethyl)pyrazin-2-yl)methyl)-3,4-dihydroquinoxalin-2-yl)piperidine-1-carboxylate (100 mg, 0.199 mmol, 1 equiv.) as the starting material to give the crude product 7-methyl-3-(piperidin-4-yl)-1-((3-(trifluoromethyl)pyrazin-2-yl)methyl)quinoxalin-2(1H)-one (70 mg) as an off-white solid. MS m/z: 404 [M+H]⁺.

Step 7: 3-(1-(2-fluoro-6-methylphenyl)piperidin-4-yl)-7-methyl-1-((3-(trifluoromethyl)pyrazin-2-yl)methyl)quinoxalin-2(1H)-one and 3-(1-(2-fluoro-6-methylphenyl)piperidin-4-yl)-6-methyl-1-((3-(trifluoromethyl)pyrazin-2-yl)methyl)quinoxalin-2(1H)-one: Followed the General Procedure G using 7-methyl-3-(piperidin-4-yl)-1-((3-(trifluoromethyl)pyrazin-2-yl)methyl)quinoxalin-2(1H)-one (70 mg, 0.174 mmol, 1.0 equiv.) and 2-bromo-1-fluoro-3-methylbenzene (49 mg, 0.261 mmol, 1.5 equiv.) as the starting materials to give 3-(1-(2-fluoro-6-methylphenyl)piperidin-4-yl)-7-methyl-1-((3-(trifluoromethyl)pyrazin-2-yl)methyl)quinoxalin-2(1H)-one (9 mg, 10.1%) as an off-white solid and 3-(1-(2-fluoro-6-methylphenyl)piperidin-4-yl)-6-methyl-1-((3-(trifluoromethyl)pyrazin-2-yl)methyl)quinoxalin-2(1H)-one (15.1 mg, 17%) as a white solid.

144A: 3-(1-(2-fluoro-6-methylphenyl)piperidin-4-yl)-7-methyl-1-((3-(trifluoromethyl)pyrazin-2-yl)methyl)quinoxalin-2(1H)-one. ¹H NMR (400 MHz, Chloroform-d) δ 8.70 (dd, J=40.4, 2.3 Hz, 2H), 7.82 (s, 1H), 7.62 (d, J=8.4 Hz, 1H), 7.42 (dd, J=8.4, 2.0 Hz, 1H), 7.01-6.94 (m, 2H), 6.90-6.83 (m, 1H), 5.92-5.85 (m, 2H), 3.42-3.07 (m, 5H), 2.53 (s, 3H), 2.38 (s, 3H), 2.21-1.99 (m, 4H). MS m/z: 512.20 [M+H]⁺.

144B: 3-(1-(2-fluoro-6-methylphenyl)piperidin-4-yl)-6-methyl-1-((3-(trifluoromethyl)pyrazin-2-yl)methyl)quinoxalin-2(1H)-one. ¹H NMR (400 MHz, Chloroform-d) δ 8.70 (dd, J=40.8, 2.4 Hz, 2H), 7.89 (d, J=8.4 Hz, 1H), 7.50 (s, 1H), 7.38 (dd, J=8.4, 2.0 Hz, 1H), 7.01-6.92 (m, 2H), 6.91-6.80 (m, 1H), 5.89 (s, 2H), 3.40-3.21 (m, 3H), 3.14-3.05 (m, 2H), 2.51 (s, 3H), 2.35 (s, 3H), 2.18-1.98 (m, 4H). MS m/z: 512.25 [M+H]⁺.

### 3-Methyl-7-(1-(5-methylisothiazol-4-yl)piperidin-4-yl)-5-((3-(trifluoromethyl)pyridin-2-yl)methyl)pyrido[2,3-b]pyrazin-6(5H)-one (145)

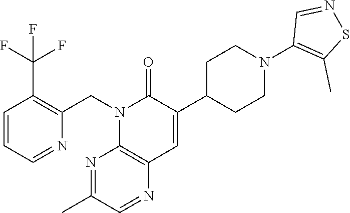 [SMILES: Cc1cnc2cc(C3CCN(c4cnsc4C)CC3)c(=O)n(Cc3ncccc3C(F)(F)F)c2n1]

Step 1: 4-bromo-5-methylisothiazole: Followed the General Procedure AN using 4-bromoisothiazole (500 mg, 3.04 mmol, 1 equiv) and iodomethane (649 mg, 4.57 mmol, 1.5 equiv) as the starting materials to give 4-bromo-5-methylisothiazole (150 mg, 27%) as a yellow oil. MS m/z: 178 [M+H]⁺.

Step 2: 3-methyl-7-(1-(5-methylisothiazol-4-yl)piperidin-4-yl)-5-((3-(trifluoromethyl)pyridin-2-yl)methyl)pyrido[2,3-b]pyrazin-6(5H)-one. Followed the General Procedure G using 3-methyl-7-(piperidin-4-yl)-5-((3-(trifluoromethyl)pyridin-2-yl)methyl)pyrido[2,3-b]pyrazin-6(5H)-one (80 mg, 0.198 mmol, 1 equiv) and 4-bromo-5-methylisothiazole (52.9 mg, 0.297 mmol, 1.5 equiv) as the starting materials to give 3-methyl-7-(1-(5-methylisothiazol-4-yl)piperidin-4-yl)-5-((3-(trifluoromethyl) pyridin-2-yl)methyl)pyrido[2,3-b]pyrazin-6(5H)-one (55.8 mg, 55.3%) as a light yellow solid. ¹H NMR (400 MHz, Chloroform-d) δ 8.42 (d, J=4.9 Hz, 1H), 8.34 (s, 2H), 7.98-7.96 (m, 1H), 7.85 (s, 1H), 7.25-7.22 (m, 1H), 6.03 (s, 2H), 3.34 (s, 2H), 3.15 (t, J=12.3 Hz, 1H), 2.98 (s, 2H), 2.52 (s, 3H), 2.47 (s, 3H), 2.12 (d, J=12.3 Hz, 2H), 2.03 (d, J=15.1 Hz, 2H). MS m/z: 501.15 [M+H]⁺.

### 5-((3-(Difluoromethyl)pyrazin-2-yl)methyl)-7-((1s,4s)-4-(2-fluoro-4-methylpyridin-3-yl)cyclohexyl)-3-methylpyrido[2,3-b]pyrazin-6(5H)-one (146A); 5-((3-(Difluoromethyl)pyrazin-2-yl)methyl)-7-((1r,4r)-4-(2-fluoro-4-methylpyridin-3-yl)cyclohexyl)-3-methylpyrido[2,3-b]pyrazin-6(5H)-one (146B)

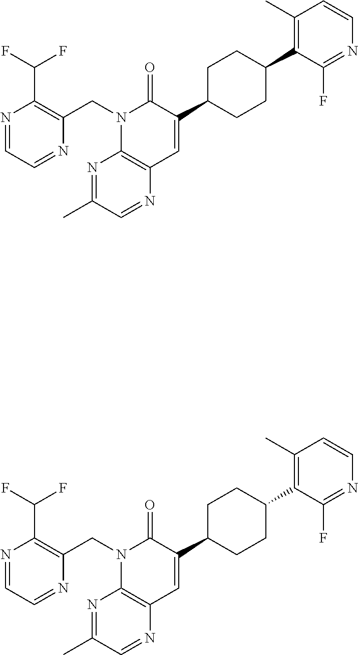 [SMILES: Cc1cnc2cc([C@H]3CC[C@@H](c4c(C)ccnc4F)CC3)c(=O)n(Cc3nccnc3C(F)F)c2n1 ; Cc1cnc2cc([C@H]3CC[C@H](c4c(C)ccnc4F)CC3)c(=O)n(Cc3nccnc3C(F)F)c2n1]

Step 1: 2-(difluoromethyl)-3-methylpyrazine: Followed the General Procedure AE using 3-methylpyrazine-2-carbaldehyde (500 mg, 4.09 mmol, 1.0 equiv.) as the starting material to give 2-(difluoromethyl)-3-methylpyrazine (300 mg, 50.8%) as a light yellow oil. MS m/z: 145 [M+H]⁺.

Step 2: 2-(bromomethyl)-3-(difluoromethyl)pyrazine: Followed the General Procedure AF using 2-(difluoromethyl)-3-methylpyrazine (300 mg, 2.08 mmol, 1.0 equiv.) as the starting material to give 2-(bromomethyl)-3-(difluoromethyl)pyrazine (100 mg, 21.5%) as a light yellow solid. MS m/z: 224 [M+H]⁺.

Step 3: 5-((3-(difluoromethyl)pyrazin-2-yl)methyl)-7-(4-(2-fluoro-4-methylpyridin-3-yl)cyclohexyl)-3-methylpyrido[2,3-b]pyrazin-6(5H)-one: Followed the General Procedure E using 2-(bromomethyl)-3-(difluoromethyl)pyrazine (100 mg, 0.448 mmol, 1.0 equiv.) and 7-(4-(2-fluoro-4-methylpyridin-3-yl)cyclohexyl)-3-methylpyrido[2,3-b]pyrazin-6(5H)-one (158 mg, 0.448 mmol, 1.0 equiv.) as the starting materials to give the crude product 5-((3-(difluoromethyl)pyrazin-2-yl)methyl)-7-(4-(2-fluoro-4-methylpyridin-3-yl)cyclohexyl)-3-methylpyrido[2,3-b]pyrazin-6(5H)-one (100 mg). The crude product was continue separated by Prep-HPLC with the following conditions (Column: XBridge Prep OBD C18 Column, 30*150 mm, 5 m; Mobile Phase A: Water (10 mmol/L NH₄HCO₃+0.05% NH₃H₂O), Mobile Phase B: ACN; Flow rate: 60 mL/min mL/min; Gradient: 46% B to 62% B in 14 min; Wave Length: 254 nm/220 nm nm; RT1 (min): 10.3/11.23) to afford 5-((3-(difluoromethyl)pyrazin-2-yl)methyl)-7-((1s,4s)-4-(2-fluoro-4-methylpyridin-3-yl)cyclohexyl)-3-methylpyrido[2,3-b]pyrazin-6(5H)-one (23.7 mg, 10.6%) as a white solid and 5-((3-(difluoromethyl)pyrazin-2-yl)methyl)-7-((1r,4r)-4-(2-fluoro-4-methylpyridin-3-yl)cyclohexyl)-3-methylpyrido[2,3-b]pyrazin-6(5H)-one (29.4 mg, 13.2%) as a white solid.

146A: 5-((3-(difluoromethyl)pyrazin-2-yl)methyl)-7-((1s,4s)-4-(2-fluoro-4-methylpyridin-3-yl)cyclohexyl)-3-methylpyrido[2,3-b]pyrazin-6(5H)-one. ¹H NMR (400 MHz, Chloroform-d) δ 8.48-8.33 (m, 3H), 8.16 (d, J=1.3 Hz, 1H), 7.88 (dd, J=5.0, 1.0 Hz, 1H), 7.13-6.80 (m, 2H), 6.09 (s, 2H), 3.44 (s, 1H), 2.99-2.88 (m, 1H), 2.54 (s, 3H), 2.40 (s, 3H), 2.32 (d, J=14.0 Hz, 2H), 2.17 (q, J=13.1 Hz, 2H), 2.04-1.89 (m, 2H), 1.63 (dd, J=13.6, 3.7 Hz, 2H). MS m/z: 495.20 [M+H]⁺.

146B: 5-((3-(difluoromethyl)pyrazin-2-yl)methyl)-7-((1r,4r)-4-(2-fluoro-4-methylpyridin-3-yl)cyclohexyl)-3-methylpyrido[2,3-b]pyrazin-6(5H)-one. ¹H NMR (400 MHz, Chloroform-d) δ 8.45-8.35 (m, 2H), 8.33 (s, 1H), 7.92 (d, J=1.3 Hz, 1H), 7.82 (dd, J=5.0, 1.0 Hz, 1H), 7.13-6.80 (m, 2H), 6.09 (s, 2H), 3.15 (t, J=12.0 Hz, 1H), 2.99-2.88 (m, 1H), 2.53 (s, 3H), 2.41 (s, 3H), 2.21-2.10 (m, 4H), 1.89-1.79 (m, 2H), 1.65 (dd, J=13.6, 3.7 Hz, 2H). MS m/z: 495.20 [M+H]⁺.

### 7-((1s,4s)-4-(2-Fluoro-4-methylpyridin-3-yl)cyclohexyl)-3-methyl-5-((3-(trifluoromethyl)pyridin-2-yl)methyl)pyrido[2,3-b]pyrazin-6(5H)-one (147A); 7-((1r,4r)-4-(2-Fluoro-4-methylpyridin-3-yl)cyclohexyl)-3-methyl-5-((3-(trifluoromethyl)pyridin-2-yl)methyl)pyrido[2,3-b]pyrazin-6(5H)-one (147B)

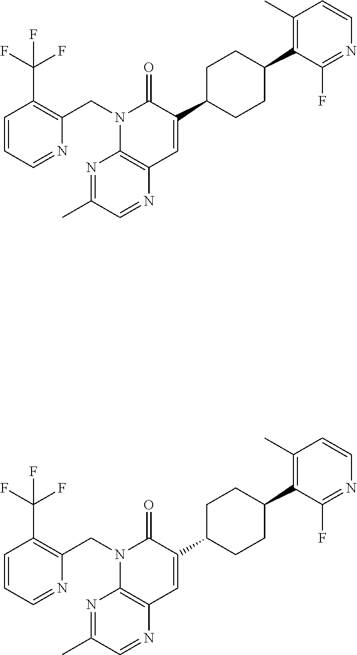 [SMILES: Cc1cnc2cc([C@H]3CC[C@@H](c4c(C)ccnc4F)CC3)c(=O)n(Cc3ncccc3C(F)(F)F)c2n1 ; Cc1cnc2cc([C@H]3CC[C@H](c4c(C)ccnc4F)CC3)c(=O)n(Cc3ncccc3C(F)(F)F)c2n1]

Step 1: 7-(4-(2-fluoro-4-methylpyridin-3-yl)cyclohexyl)-3-methyl-5-((3-(trifluoromethyl)pyridin-2-yl)methyl)pyrido[2,3-b]pyrazin-6(5H)-one: Followed the General Procedure E using 7-(4-(2-fluoro-4-methylpyridin-3-yl)cyclohexyl)-3-methylpyrido[2,3-b]pyrazin-6(5H)-one (130 mg, 0.368 mmol, 1.0 equiv.) and 2-(bromomethyl)-3-(trifluoromethyl)pyridine hydrobromide (118 mg, 0.368 mmol, 1.0 equiv.) as the starting materials to give the crude product 7-(4-(2-fluoro-4-methylpyridin-3-yl)cyclohexyl)-3-methyl-5-((3-(trifluoromethyl)pyridin-2-yl)methyl)pyrido[2,3-b]pyrazin-6(5H)-one (110 mg). The crude product was continue separated by Prep-HPLC with the following conditions (Column: Xselect CSH C18 OBD Column 30*150 mm, 5 m; Mobile Phase A: Water (0.1% FA), Mobile Phase B: ACN; Flow rate: 60 mL/min mL/min; Gradient: 70% B to 85% B in 8 min; Wave Length: 254 nm/220 nm nm; RT1 (min): 8.67/9.18) to afford 7-((1s,4s)-4-(2-fluoro-4-methylpyridin-3-yl)cyclohexyl)-3-methyl-5-((3-(trifluoromethyl)pyridin-2-yl)methyl)pyrido[2,3-b]pyrazin-6(5H)-one (21.3 mg, 7.6%) as a white solid and 7-((1r,4r)-4-(2-fluoro-4-methylpyridin-3-yl)cyclohexyl)-3-methyl-5-((3-(trifluoromethyl)pyridin-2-yl)methyl)pyrido[2,3-b]pyrazin-6(5H)-one (25.3 mg, 8.93%) as a white solid.

147A: 7-((1s,4s)-4-(2-fluoro-4-methylpyridin-3-yl)cyclohexyl)-3-methyl-5-((3-(trifluoromethyl)pyridin-2-yl)methyl)pyrido[2,3-b]pyrazin-6(5H)-one. ¹H NMR (400 MHz, Chloroform-d) δ 8.46-8.39 (m, 1H), 8.35 (s, 1H), 8.15 (d, J=1.3 Hz, 1H), 8.01-7.83 (m, 2H), 7.22 (dd, J=8.0, 4.9 Hz, 1H), 7.00-6.91 (m, 1H), 6.03 (s, 2H), 3.46 (s, 1H), 3.00-2.88 (m, 1H), 2.52 (s, 3H), 2.40 (s, 3H), 2.34 (d, J=14.1 Hz, 2H), 2.19 (q, J=13.3 Hz, 2H), 1.98 (d, J=13.9 Hz, 2H), 1.69-1.60 (m, 2H). MS m/z: 512.25 [M+H]⁺.

147B: 7-((1r,4r)-4-(2-fluoro-4-methylpyridin-3-yl)cyclohexyl)-3-methyl-5-((3-(trifluoromethyl)pyridin-2-yl)methyl)pyrido[2,3-b]pyrazin-6(5H)-one. ¹H NMR (400 MHz, Chloroform-d) δ 8.46-8.39 (m, 1H), 8.32 (s, 1H), 7.97 (dd, J=7.5, 1.6 Hz, 1H), 7.89 (d, J=4.9 Hz, 1H), 7.80 (d, J=0.9 Hz, 1H), 7.23 (dd, J=8.0, 4.9 Hz, 1H), 6.95 (d, J=5.0 Hz, 1H), 6.03 (s, 2H), 3.14 (t, J=12.0 Hz, 1H), 2.97-2.85 (m, 1H), 2.51 (s, 3H), 2.41 (s, 3H), 2.27-2.01 (m, 4H), 1.82 (dd, J=13.8, 4.1 Hz, 2H), 1.56 (s, 2H). MS m/z: 512.20 [M+H]⁺.

### 5-((3-(Difluoromethyl)pyrazin-2-yl)methyl)-7-((1s,4s)-4-(2-fluoro-6-methylphenyl)cyclohexyl)-3-methylpyrido[2,3-b]pyrazin-6(5H)-one (148A); 5-((3-(Difluoromethyl)pyrazin-2-yl)methyl)-7-((1r,4r)-4-(2-fluoro-6-methylphenyl)cyclohexyl)-3-methylpyrido[2,3-b]pyrazin-6(5H)-one (148B)

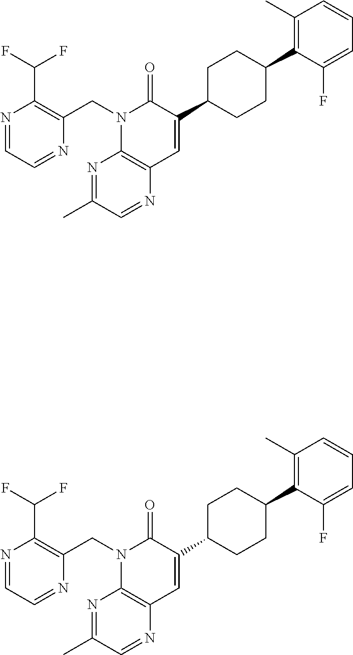 [SMILES: Cc1cnc2cc([C@H]3CC[C@@H](c4c(C)cccc4F)CC3)c(=O)n(Cc3nccnc3C(F)F)c2n1 ; Cc1cnc2cc([C@H]3CC[C@H](c4c(C)cccc4F)CC3)c(=O)n(Cc3nccnc3C(F)F)c2n1]

Step 1: 5-((3-(difluoromethyl)pyrazin-2-yl)methyl)-7-((1s,4s)-4-(2-fluoro-6-methylphenyl)cyclohexyl)-3-methylpyrido[2,3-b]pyrazin-6(5H)-one and 5-((3-(difluoromethyl)pyrazin-2-yl)methyl)-7-((1r,4r)-4-(2-fluoro-6-methylphenyl)cyclohexyl)-3-methylpyrido[2,3-b]pyrazin-6(5H)-one: Followed the General Procedure E using 7-(4-(2-fluoro-6-methylphenyl)cyclohexyl)-3-methylpyrido[2,3-b]pyrazin-6(5H)-one (90 mg, 0.256 mmol, 1.0 equiv.) and 2-(bromomethyl)-3-(difluoromethyl)pyrazine hydrochloride (72.8 mg, 0.282 mmol, 1.1 equiv.) as the starting materials to give 5-((3-(difluoromethyl)pyrazin-2-yl)methyl)-7-((1s,4s)-4-(2-fluoro-6-methylphenyl)cyclohexyl)-3-methylpyrido[2,3-b]pyrazin-6(5H)-one (14.2 mg, 11.2%) and 5-((3-(difluoromethyl)pyrazin-2-yl)methyl)-7-((1r,4r)-4-(2-fluoro-6-methylphenyl)cyclohexyl)-3-methylpyrido[2,3-b]pyrazin-6(5H)-one (25.3 mg, 20.0%) as a white solid.

148A: 5-((3-(difluoromethyl)pyrazin-2-yl)methyl)-7-((1s,4s)-4-(2-fluoro-6-methylphenyl)cyclohexyl)-3-methylpyrido[2,3-b]pyrazin-6(5H)-one. ¹H NMR (400 MHz, Chloroform-d) δ 8.45-8.34 (m, 3H), 8.21 (s, 1H), 7.12-6.96 (m, 2H), 6.94-6.77 (m, 2H), 6.09 (s, 2H), 3.50-3.41 (m, 1H), 2.93 (t, J=12.4 Hz, 1H), 2.54 (s, 3H), 2.37 (s, 3H), 2.31-2.14 (m, 4H), 2.02-1.86 (m, 2H), 1.70-1.63 (m, 2H). MS m/z: 494.20 [M+H]⁺.

148B: 5-((3-(difluoromethyl)pyrazin-2-yl)methyl)-7-((1r,4r)-4-(2-fluoro-6-methylphenyl)cyclohexyl)-3-methylpyrido[2,3-b]pyrazin-6(5H)-one. ¹H NMR (400 MHz, Chloroform-d) δ 8.50-8.40 (m, 2H), 8.34 (s, 1H), 7.84 (s, 1H), 7.12-6.96 (m, 2H), 6.94-6.82 (m, 2H), 6.10 (s, 2H), 3.13 (t, J=12.0 Hz, 1H), 2.89 (t, J=12.0 Hz, 1H), 2.54 (s, 3H), 2.38 (s, 3H), 2.21-2.10 (m, 4H), 1.89-1.82 (m, 2H), 1.53 (q, J=12.4 Hz, 2H). MS m/z: 494.20 [M+H]⁺.

### (R)-5-(8,8-Difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one (149A); (S)-5-(8,8-Difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one (149B)

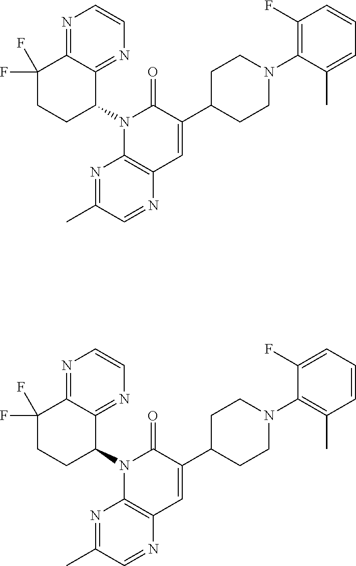 [SMILES: Cc1cnc2cc(C3CCN(c4c(C)cccc4F)CC3)c(=O)n([C@@H]3CCC(F)(F)c4nccnc43)c2n1 ; Cc1cnc2cc(C3CCN(c4c(C)cccc4F)CC3)c(=O)n([C@H]3CCC(F)(F)c4nccnc43)c2n1]

Step 1: 5,6,7,8-tetrahydroquinoxaline 1-oxide: To a stirred solution of 5,6,7,8-tetrahydroquinoxaline (4000 mg, 29.6 mmol, 1.0 equiv.) in DCM (20 mL) was added m-CPBA (5625 mg, 32.6 mmol, 1.1 equiv.) dropwise at 0° C. under argon atmosphere. The resulting mixture was stirred for overnight at room temperature under argon atmosphere. Aqueous 10% Na₂S₂O₃ (14 mL) was added dropwise in 5 min. Next, saturated aq. NaHCO₃ (48 mL) was added in 10 min. The mixture was stirred for an additional 60 min until no more gas evolved. The resulting mixture was extracted with CH₂Cl₂ (3×40 mL). The combined organic layers were washed with brine (2×10 mL), dried over anhydrous Na₂SO₄. After filtration, the filtrate was concentrated under reduced pressure. The crude product 5,6,7,8-tetrahydroquinoxaline 1-oxide (4 g) was used in the next step directly without further purification. MS m/z: 151 [M+H]⁺.

Step 2: 5,6,7,8-tetrahydroquinoxalin-5-ol: To a stirred solution of 5,6,7,8-tetrahydroquinoxaline 1-oxide (4 g, 26.5 mmol, 1.0 equiv.) in DCM (40 mL) were added TFAA (16.7 g, 79.5 mmol, 3.0 equiv.) dropwise at 0° C. under argon atmosphere. The resulting mixture was stirred for overnight at room temperature under argon atmosphere. The resulting mixture was concentrated under reduced pressure. The residue was dissolved in CH₂Cl₂ (40 mL). To the above mixture was added 2 N aqueous LiOH (27 mL) dropwise at room temperature. The resulting mixture was stirred for additional 1 h at room temperature. The resulting mixture was extracted with CH₂Cl₂ (3×40 mL). The combined organic layers were washed with brine (2×10 mL), dried over anhydrous Na₂SO₄. After filtration, the filtrate was concentrated under reduced pressure. The residue was purified by silica gel column chromatography, eluted with CH₂Cl₂/MeOH (20:1) to afford 5,6,7,8-tetrahydroquinoxalin-5-ol (850 mg, 21%) as a brown oil. MS m/z: 151 [M+H]⁺.

Step 3: 7,8-dihydroquinoxalin-5(6H)-one: Followed the General Procedure AM using 5,6,7,8-tetrahydroquinoxalin-5-ol (850 mg, 5.63 mmol, 1.0 equiv.) as the starting material to give 7,8-dihydroquinoxalin-5(6H)-one (280 mg, 33%) as a brown oil. MS m/z: 149 [M+H]⁺.

Step 4: 5,5-difluoro-5,6,7,8-tetrahydroquinoxaline: Followed the General Procedure AE using 7,8-dihydroquinoxalin-5(6H)-one (280 mg, 1.88 mmol, 1.0 equiv.) as the starting material to give 5,5-difluoro-5,6,7,8-tetrahydroquinoxaline (170 mg, 52%) as a brown oil. MS m/z: 171 [M+H]⁺.

Step 5: 8-bromo-5,5-difluoro-5,6,7,8-tetrahydroquinoxaline: Followed the General Procedure AF using 5,5-difluoro-5,6,7,8-tetrahydroquinoxaline (170 mg, 0.994 mmol, 1.0 equiv.) as the starting material to give 8-bromo-5,5-difluoro-5,6,7,8-tetrahydroquinoxaline (185 mg, 74%) as a brown oil. MS m/z: 249 [M+H]⁺.

Step 6: tert-butyl 4-(5-(8,8-difluoro-5,6,7,8-tetrahydroquinoxalin-5-yl)-3-methyl-6-oxo-5,6-dihydropyrido[2,3-b]pyrazin-7-yl)piperidine-1-carboxylate: Followed the General Procedure E using 8-bromo-5,5-difluoro-5,6,7,8-tetrahydroquinoxaline (185 mg, 0.743 mmol, 1.0 equiv.) and tert-butyl 4-(3-methyl-6-oxo-5,6-dihydropyrido[2,3-b]pyrazin-7-yl)piperidine-1-carboxylate (282 mg, 0.717 mmol, 1.1 equiv.) as the starting material to give tert-butyl 4-(5-(8,8-difluoro-5,6,7,8-tetrahydroquinoxalin-5-yl)-3-methyl-6-oxo-5,6-dihydropyrido[2,3-b]pyrazin-7-yl)piperidine-1-carboxylate (200 mg, 52%). MS m/z: 513 [M+H]⁺.

Step 7: 5-(8,8-difluoro-5,6,7,8-tetrahydroquinoxalin-5-yl)-3-methyl-7-(piperidin-4-yl)pyrido[2,3-b]pyrazin-6(5H)-one: Followed the General Procedure F using tert-butyl 4-(5-(8,8-difluoro-5,6,7,8-tetrahydroquinoxalin-5-yl)-3-methyl-6-oxo-5,6-dihydropyrido[2,3-b]pyrazin-7-yl)piperidine-1-carboxylate (200 mg, 0.340 mmol, 1.0 equiv.) as the starting material to give the crude product 5-(8,8-difluoro-5,6,7,8-tetrahydroquinoxalin-5-yl)-3-methyl-7-(piperidin-4-yl)pyrido[2,3-b]pyrazin-6(5H)-one (180 mg). MS m/z: 413 [M+H]⁺.

Step 8: 5-(8,8-difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one: Followed the General Procedure G using 5-(8,8-difluoro-5,6,7,8-tetrahydroquinoxalin-5-yl)-3-methyl-7-(piperidin-4-yl)pyrido[2,3-b]pyrazin-6(5H)-one (180 mg, 0.436 mmol, 1.0 equiv.) and 2-bromo-1-fluoro-3-methylbenzene (82.5 mg, 0.436 mmol, 1.0 equiv.) as the starting materials to give the crude product 5-(8,8-difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one (110 mg) as a white solid. The crude product was purified by Chiral-HPLC with the following conditions (Column: CHIRAL ART Cellulose-SB 4.6*100 mm, 3 m; Mobile Phase B: MeOH (0.1% DEA); Gradient: isocratic % B) to afford (R)-5-(8,8-difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one (15.8 mg, 6.79%) as a white solid and (S)-5-(8,8-difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one (9.5 mg, 4.03%) as a white solid.

149A: (R)-5-(8,8-difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one. ¹H NMR (300 MHz, DMSO-d₆) δ 8.58 (dd, J=17.8, 8.9 Hz, 2H), 8.37 (d, J=40.6 Hz, 1H), 7.84 (s, 1H), 7.00 (d, J=21.9 Hz, 4H), 3.23 (s, 3H), 2.67 (s, 5H), 2.35 (d, J=11.8 Hz, 4H), 2.21-1.33 (m, 7H). MS m/z: 521.25 [M+H]⁺.

149B: (S)-5-(8,8-difluoro-5,6,7,8-tetrahydroquinoxalin-5-yl)-7-(1-(2-fluoro-6-methylphenyl)piperidin-4-yl)-3-methylpyrido[2,3-b]pyrazin-6(5H)-one. ¹H NMR (300 MHz, DMSO-d₆) δ 8.59 (dd, J=18.4, 9.0 Hz, 2H), 8.37 (d, J=40.5 Hz, 1H), 7.84 (d, J=0.9 Hz, 1H), 6.99 (d, J=22.0 Hz, 4H), 3.34-3.11 (m, 3H), 2.74 (d, J=43.9 Hz, 5H), 2.35 (d, J=12.1 Hz, 4H), 2.05 (d, J=31.1 Hz, 3H), 1.96-1.52 (m, 4H). MS m/z: 521.3 [M+H]⁺.

### 3-Methyl-5-((3-(trifluoromethyl)pyridin-2-yl)methyl)-7-((1s,4s)-4-(4-(trifluoromethyl)pyridin-3-yl)cyclohexyl)pyrido[2,3-b]pyrazin-6(5H)-one (150A); 3-Methyl-5-((3-(trifluoromethyl)pyridin-2-yl)methyl)-7-((1r,4r)-4-(4-(trifluoromethyl)pyridin-3-yl)cyclohexyl)pyrido[2,3-b]pyrazin-6(5H)-one (150B)

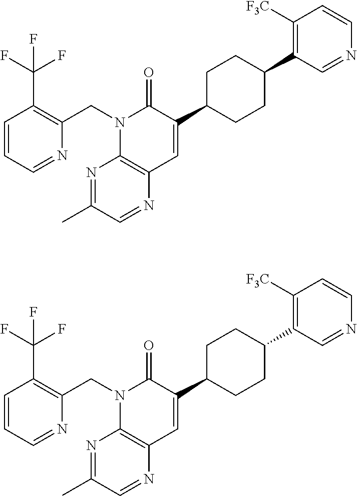 [SMILES: Cc1cnc2cc([C@H]3CC[C@@H](c4cnccc4C(F)(F)F)CC3)c(=O)n(Cc3ncccc3C(F)(F)F)c2n1 ; Cc1cnc2cc([C@H]3CC[C@H](c4cnccc4C(F)(F)F)CC3)c(=O)n(Cc3ncccc3C(F)(F)F)c2n1]

Step 1: ethyl 2-(4-(4-(trifluoromethyl)pyridin-3-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (500 mg, 1.70 mmol, 1 equiv.) and 3-bromo-4-(trifluoromethyl)pyridine (453 mg, 2.55 mmol, 1.5 equiv.) as the starting materials to give ethyl 2-(4-(4-(trifluoromethyl)pyridin-3-yl)cyclohex-3-en-1-yl)acetate (450 mg, 84.5%) as a light yellow oil. MS m/z: 314 [M+H]⁺.

Step 2: ethyl 2-(4-(4-(trifluoromethyl)pyridin-3-yl)cyclohexyl)acetate: Followed the General Procedure AR using 2-(4-(4-(trifluoromethyl)pyridin-3-yl)cyclohex-3-en-1-yl)acetate (450 mg, 1.70 mmol, 1 equiv.) as the starting material to give ethyl 2-(4-(4-(trifluoromethyl)pyridin-3-yl)cyclohexyl)acetate (280 mg, 61%) as a colorless oil. MS m/z: 316 [M+H]⁺.

Step 3: 3-methyl-7-(4-(4-(trifluoromethyl)pyridin-3-yl)cyclohexyl)pyrido[2,3-b]pyrazin-6(5H)-one: Followed the General Procedure D using ethyl 2-(4-(4-(trifluoromethyl)pyridin-3-yl)cyclohexyl)acetate (280 mg, 1.04 mmol, 1 equiv.) and 3-amino-5-methylpyrazine-2-carbaldehyde (215 mg, 1.57 mmol, 1.5 equiv.) as the starting materials to give 3-methyl-7-(4-(4-(trifluoromethyl)pyridin-3-yl)cyclohexyl)pyrido[2,3-b]pyrazin-6(5H)-one (100 mg, 28%) as an off-white solid. MS m/z: 389 [M+H]⁺.

Step 4: 3-methyl-5-((3-(trifluoromethyl)pyridin-2-yl)methyl)-7-((1s,4s)-4-(4-(trifluoromethyl)pyridin-3-yl)cyclohexyl)pyrido[2,3-b]pyrazin-6(5H)-one and 3-methyl-5-((3-(trifluoromethyl)pyridin-2-yl)methyl)-7-((1r,4r)-4-(4-(trifluoromethyl)pyridin-3-yl)cyclohexyl)pyrido[2,3-b]pyrazin-6(5H)-one: Followed the General Procedure E using 3-methyl-7-(4-(4-(trifluoromethyl)pyridin-3-yl)cyclohexyl)pyrido[2,3-b]pyrazin-6(5H)-one (100 mg, 0.257 mmol, 1.0 equiv.) and 2-(chloromethyl)-3-(trifluoromethyl)pyridine (75.4 mg, 0.386 mmol, 1.5 equiv.) as the starting materials to give 3-methyl-5-((3-(trifluoromethyl)pyridin-2-yl)methyl)-7-((1s,4s)-4-(4-(trifluoromethyl)pyridin-3-yl)cyclohexyl)pyrido[2,3-b]pyrazin-6(5H)-one (11.4 mg, 8.0%) as an off-white solid and 3-methyl-5-((3-(trifluoromethyl)pyridin-2-yl)methyl)-7-((1r,4r)-4-(4-(trifluoromethyl)pyridin-3-yl)cyclohexyl)pyrido[2,3-b]pyrazin-6(5H)-one (7.3 mg, 5.0%) as an off-white solid.

150A: 3-methyl-5-((3-(trifluoromethyl)pyridin-2-yl)methyl)-7-((1s,4s)-4-(4-(trifluoromethyl)pyridin-3-yl)cyclohexyl)pyrido[2,3-b]pyrazin-6(5H)-one. ¹H NMR (400 MHz, Chloroform-d) δ 8.77 (s, 1H), 8.59 (d, J=5.2 Hz, 1H), 8.41 (d, J=4.8 Hz, 1H), 8.36 (s, 1H), 8.09 (s, 1H), 7.97 (d, J=7.6 Hz, 1H), 7.48 (d, J=5.2 Hz, 1H), 7.23 (dd, J=7.6, 4.9 Hz, 1H), 6.04 (s, 2H), 3.49-3.39 (m, 1H), 3.14-3.02 (m, 1H), 2.53 (s, 3H), 2.36 (d, J=14.0 Hz, 2H), 2.07-1.97 (m, 2H), 1.92-1.81 (m, 4H). MS m/z: 548.25 [M+H]⁺.

150B: 3-methyl-5-((3-(trifluoromethyl)pyridin-2-yl)methyl)-7-((1r,4r)-4-(4-(trifluoromethyl)pyridin-3-yl)cyclohexyl)pyrido[2,3-b]pyrazin-6(5H)-one. ¹H NMR (400 MHz, Chloroform-d) δ 8.81 (s, 1H), 8.61 (d, J=5.2 Hz, 1H), 8.43 (d, J=4.8 Hz, 1H), 8.34 (s, 1H), 7.97 (d, J=8.0 Hz, 1H), 7.82 (s, 1H), 7.51 (d, J=5.2 Hz, 1H), 7.26-7.21 (m, 1H), 6.04 (s, 2H), 3.16 (t, J=12.0 Hz, 1H), 3.04 (t, J=12.4 Hz, 1H), 2.52 (s, 3H), 2.21 (d, J=12.8 Hz, 2H), 2.08-2.00 (m, 2H), 1.89-1.77 (m, 2H), 1.67-1.54 (m, 2H). MS m/z: 548.10 [M+H]⁺.

### 3-Methyl-7-(1-(3-methylpyrazin-2-yl)piperidin-4-yl)-5-((3-(trifluoromethyl)pyridin-2-yl)methyl)pyrido[2,3-b]pyrazin-6(5H)-one (151)

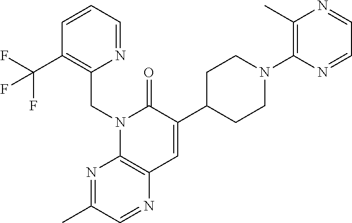 [SMILES: Cc1cnc2cc(C3CCN(c4nccnc4C)CC3)c(=O)n(Cc3ncccc3C(F)(F)F)c2n1]

Step 1: 3-methyl-7-(1-(3-methylpyrazin-2-yl)piperidin-4-yl)-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (50 mg, 0.124 mmol, 1 equiv) and 2-bromo-3-methylpyrazine (25.73 mg, 0.149 mmol, 1.2 equiv) as the starting materials to give 3-methyl-7-(1-(3-methylpyrazin-2-yl)piperidin-4-yl)-5-((3-(trifluoromethyl)pyridin-2-yl)methyl)pyrido[2,3-b]pyrazin-6(5H)-one (26.0 mg, 41.9%) as a white solid. ¹H NMR (400 MHz, Chloroform-d) δ 8.43 (d, J=4.9 Hz, 1H), 8.34 (s, 1H), 8.09-8.02 (m, 2H), 7.97 (d, J=7.9 Hz, 1H), 7.84 (s, 1H), 7.25-7.21 (m, 1H), 6.03 (s, 2H), 3.72 (d, J=12.7 Hz, 2H), 3.28-3.18 (m, 1H), 3.05-2.96 (m, 2H), 2.56 (s, 3H), 2.52 (s, 3H), 2.14 (d, J=12.5 Hz, 2H), 1.89-1.77 (m, 2H). MS m/z: 496.15 [M+H]⁺.

### 7-((1r,4r)-4-(2-Fluoro-6-methylphenyl)cyclohexyl)-3-methyl-5-((3-(trifluoromethoxy)pyridin-2-yl)methyl)pyrido[2,3-b]pyrazin-6(5H)-one (152)

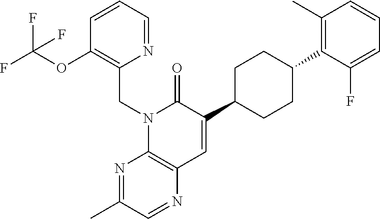 [SMILES: Cc1cnc2cc([C@H]3CC[C@H](c4c(C)cccc4F)CC3)c(=O)n(Cc3ncccc3OC(F)(F)F)c2n1]

Step 1: 7-((1r,4r)-4-(2-fluoro-6-methylphenyl)cyclohexyl)-3-methyl-5-((3-(trifluoromethoxy)pyridin-2-yl)methyl)pyrido[2,3-b]pyrazin-6(5H)-one: Followed the General Procedure E using 7-(4-(2-fluoro-6-methylphenyl)cyclohexyl)pyrido[2,3-b]pyrazin-6(5H)-one (100 mg, 0.285 mmol, 1 equiv.) and 2-(chloromethyl)-3-(trifluoromethoxy)pyridine (78.3 mg, 0.370 mmol, 1.3 equiv.) as the starting materials to give 7-((1r,4r)-4-(2-fluoro-6-methylphenyl)cyclohexyl)-3-methyl-5-((3-(trifluoromethoxy)pyridin-2-yl)methyl)pyrido[2,3-b]pyrazin-6(5H)-one (65.1 mg, 43.4%) as a white solid. ¹H NMR (400 MHz, Chloroform-d) δ 8.35-8.26 (m, 2H), 7.82 (s, 1H), 7.62 (d, J=8.4 Hz, 1H), 7.25-7.19 (m, 1H), 7.08-6.98 (m, 1H), 6.95-6.90 (m, 1H), 6.85 (dd, J=12.0, 8.1 Hz, 1H), 5.96 (s, 2H), 3.14 (t, J=12.0 Hz, 1H), 2.90 (t, J=12.4 Hz, 1H), 2.54 (s, 3H), 2.38 (s, 3H), 2.24-2.10 (m, 4H), 1.88-1.82 (m, 2H), 1.53 (q, J=12.4 Hz, 2H). MS m/z: 527.15 [M+H]⁺.

### 3-Methyl-7-(1-(3-methylpyrazin-2-yl)piperidin-4-yl)-5-((3-(trifluoromethoxy)pyridin-2-yl)methyl)pyrido[2,3-b]pyrazin-6(5H)-one (153)

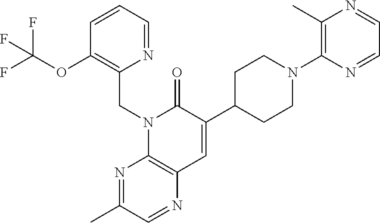 [SMILES: Cc1cnc2cc(C3CCN(c4nccnc4C)CC3)c(=O)n(Cc3ncccc3OC(F)(F)F)c2n1]

Step 1: tert-butyl 4-(3-methyl-6-oxo-5-((3-(trifluoromethoxy)pyridin-2-yl)methyl)-5,6-dihydropyrido[2,3-b]pyrazin-7-yl)piperidine-1-carboxylate: Followed the General Procedure E using tert-butyl 4-(3-methyl-6-oxo-5,6-dihydropyrido[2,3-b]pyrazin-7-yl)piperidine-1-carboxylate (100 mg, 290 μmol, 1.0 equiv) and 2-(chloromethyl)-3-(trifluoromethoxy)pyridine hydrochloride (71.8 mg, 290 μmol, 1.0 equiv.) as the starting materials to give tert-butyl 4-(3-methyl-6-oxo-5-((3-(trifluoromethoxy)pyridin-2-yl)methyl)-5,6-dihydropyrido[2,3-b]pyrazin-7-yl)piperidine-1-carboxylate (70 mg, 46%) as a white solid. MS m/z: 520 [M+H]⁺.

Step 2: 3-methyl-7-(piperidin-4-yl)-5-((3-(trifluoromethoxy)pyridin-2-yl)methyl)pyrido[2,3-b]pyrazin-6(5H)-one: Followed the General Procedure F using tert-butyl 4-(3-methyl-6-oxo-5-((3-(trifluoromethoxy)pyridin-2-yl)methyl)-5,6-dihydropyrido[2,3-b]pyrazin-7-yl)piperidine-1-carboxylate (70 mg) as the starting material to give the crude product 3-methyl-7-(piperidin-4-yl)-5-((3-(trifluoromethoxy)pyridin-2-yl)methyl)pyrido[2,3-b]pyrazin-6(5H)-one (60 mg). MS m/z: 420 [M+H]⁺.

Step 3: 3-methyl-7-(1-(3-methylpyrazin-2-yl)piperidin-4-yl)-5-((3-(trifluoromethoxy)pyridin-2-yl)methyl)pyrido[2,3-b]pyrazin-6(5H)-one: Followed the General Procedure G using 3-methyl-7-(piperidin-4-yl)-5-((3-(trifluoromethoxy)pyridin-2-yl)methyl)pyrido[2,3-b]pyrazin-6(5H)-one (60 mg, 143 μmol, 1.0 equiv) and 2-bromo-3-methylpyrazine (24 mg, 143 μmol, 1.0 equiv) as the starting materials to give 3-methyl-7-(1-(3-methylpyrazin-2-yl)piperidin-4-yl)-5-((3-(trifluoromethoxy)pyridin-2-yl)methyl)pyrido[2,3-b]pyrazin-6(5H)-one (10.4 mg, 7.2%) as a white solid. ¹H NMR (400 MHz, Chloroform-d) δ 8.34 (s, 1H), 8.26 (dd, J=4.7, 1.4 Hz, 1H), 8.12 (s, 1H), 8.03 (d, J=2.7 Hz, 1H), 7.82 (d, J=1.0 Hz, 1H), 7.58 (dq, J=8.3, 1.6 Hz, 1H), 7.20 (dd, J=8.3, 4.7 Hz, 1H), 5.95 (s, 2H), 3.75 (d, J=12.7 Hz, 2H), 3.23 (t, J=12.1 Hz, 1H), 3.03 (t, J=12.4 Hz, 2H), 2.65-2.57 (m, 3H), 2.53 (s, 3H), 2.14 (d, J=12.6 Hz, 2H), 1.82 (qd, J=12.4, 3.7 Hz, 2H). MS m/z: 512.2 [M+H]⁺.

### 7-(1-(3-(Difluoromethyl)pyridin-2-yl)piperidin-4-yl)-3-methyl-5-((3-(trifluoromethyl)pyridin-2-yl)methyl)pyrido[2,3-b]pyrazin-6(5H)-one (154)

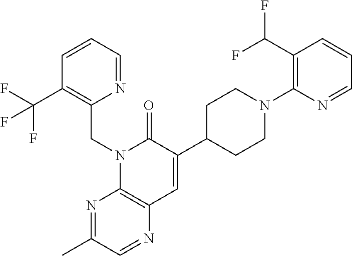 [SMILES: Cc1cnc2cc(C3CCN(c4ncccc4C(F)F)CC3)c(=O)n(Cc3ncccc3C(F)(F)F)c2n1]

Step 1: 7-(1-(3-(difluoromethyl)pyridin-2-yl)piperidin-4-yl)-3-methyl-5-((3-(trifluoromethyl)pyridin-2-yl)methyl)pyrido[2,3-b]pyrazin-6(5H)-one: Followed General Procedure G using 3-methyl-7-(piperidin-4-yl)-5-((3-(trifluoromethyl)pyridin-2-yl)methyl)pyrido[2,3-b]pyrazin-6(5H)-one (50 mg, 0.124 mmol, 1 equiv) and 2-bromo-3-(difluoromethyl)pyridine (30.94 mg, 0.149 mmol, 1.2 equiv) as the starting materials to give 7-(1-(3-(difluoromethyl)pyridin-2-yl)piperidin-4-yl)-3-methyl-5-((3-(trifluoromethyl)pyridin-2-yl)methyl)pyrido[2,3-b]pyrazin-6(5H)-one (25.8 mg, 39.0%) as a white solid. ¹H NMR (400 MHz, Chloroform-d) δ 8.42 (d, J=4.8 Hz, 2H), 8.34 (s, 1H), 8.00-7.89 (m, 2H), 7.84 (d, J=1.0 Hz, 1H), 7.23 (dd, J=8.0, 4.9 Hz, 1H), 7.06 (dd, J=7.6, 4.9 Hz, 1H), 6.86 (t, J=54.9 Hz, 1H), 6.03 (s, 2H), 3.59 (d, J=12.5 Hz, 2H), 3.17 (ddd, J=26.4, 12.9, 10.0 Hz, 3H), 2.51 (s, 3H), 2.13 (d, J=12.3 Hz, 2H), 1.85 (md, J=12.4, 3.7 Hz, 2H). MS m/z:531.25 [M+H]⁺.

### 5-((3-(Difluoromethoxy)pyrazin-2-yl)methyl)-7-(1-(2-fluoro-6-methylphenyl)piperidin-4-yl)pyrido[2,3-b]pyrazin-6(5H)-one (155)

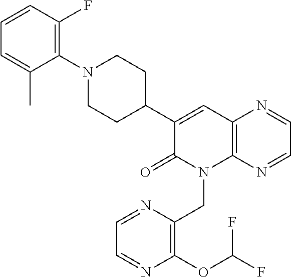 [SMILES: Cc1cccc(F)c1N1CCC(c2cc3nccnc3n(Cc3nccnc3OC(F)F)c2=O)CC1]

Step 1: 7-(1-(2-fluoro-6-methylphenyl)piperidin-4-yl)pyrido[2,3-b]pyrazin-6(5H)-one: Followed the General Procedure D using methyl 2-(1-(2-fluoro-6-methylphenyl)piperidin-4-yl)acetate (300 mg, 1.13 mmol, 1 equiv) and 3-aminopyrazine-2-carbaldehyde (155 mg, 1.13 mmol, 1 equiv) as the starting materials to give 7-(1-(2-fluoro-6-methylphenyl)piperidin-4-yl)pyrido[2,3-b]pyrazin-6(5H)-one (150 mg, 52%) as a yellow solid. MS m/z: 339 [M+H]⁺.

Step 2: 7-(1-(2-fluoro-6-methylphenyl)piperidin-4-yl)-5-((3-((tetrahydro-2H-pyran-2-yl)oxy)pyrazin-2-yl)methyl)pyrido[2,3-b]pyrazin-6(5H)-one: Followed the General Procedure E using 7-(1-(2-fluoro-6-methylphenyl)piperidin-4-yl)pyrido[2,3-b]pyrazin-6(5H)-one (150 mg, 0.443 mmol, 1 equiv) and 2-(bromomethyl)-3-((tetrahydro-2H-pyran-2-yl)oxy)pyrazine (241 mg, 0.886 mmol, 2 equiv) as the starting materials to give the 7-(1-(2-fluoro-6-methylphenyl)piperidin-4-yl)-5-((3-((tetrahydro-2H-pyran-2-yl)oxy)pyrazin-2-yl)methyl)pyrido[2,3-b]pyrazin-6(5H)-one (80 mg, 34%) as a white solid. MS m/z: 531 [M+H]⁺.

Step 3: 7-(1-(2-fluoro-6-methylphenyl)piperidin-4-yl)-5-((3-hydroxypyrazin-2-yl)methyl)pyrido[2,3-b]pyrazin-6(5H)-one: Followed the General Procedure F using 7-(1-(2-fluoro-6-methylphenyl)piperidin-4-yl)-5-((3-((tetrahydro-2H-pyran-2-yl)oxy)pyrazin-2-yl)methyl)pyrido[2,3-b]pyrazin-6(5H)-one (80 mg, 0.151 mmol, 1 equiv) as the starting material to give the crude product 7-(1-(2-fluoro-6-methylphenyl)piperidin-4-yl)-5-((3-hydroxypyrazin-2-yl)methyl)pyrido[2,3-b]pyrazin-6(5H)-one (80 mg) as a white solid. MS m/z: 461 [M+H]⁺.

Step 4: 5-((3-(difluoromethoxy)pyrazin-2-yl)methyl)-7-(1-(2-fluoro-6-methylphenyl)piperidin-4-yl)pyrido[2,3-b]pyrazin-6(5H)-one: Followed the General Procedure AU using 7-(1-(2-fluoro-6-methylphenyl)piperidin-4-yl)-5-((3-hydroxypyrazin-2-yl)methyl)pyrido[2,3-b]pyrazin-6(5H)-one (80 mg, 0.179 mmol, 1 equiv) and methyl 2-chloro-2,2-difluoroacetate (51.5 mg, 0.358 mmol, 2 equiv) as the starting materials to give 5-((3-(difluoromethoxy)pyrazin-2-yl)methyl)-7-(1-(2-fluoro-6-methylphenyl)piperidin-4-yl)pyrido[2,3-b]pyrazin-6(5H)-one (28 mg, 31.4%) as a white solid. ¹H NMR (400 MHz, Chloroform-d) δ 8.48 (d, J=2.4 Hz, 1H), 8.36 (d, J=2.4 Hz, 1H), 8.09 (d, J=2.7 Hz, 1H), 7.99 (d, J=2.7 Hz, 1H), 7.89 (s, 1H), 7.51 (t, J=71.9 Hz, 1H), 7.00-6.95 (m, 2H), 6.92-6.82 (m, 1H), 5.91 (s, 2H), 3.41-3.28 (m, 2H), 3.23-3.06 (m, 3H), 2.37 (s, 3H), 2.08-2.01 (m, 2H), 1.92-1.77 (m, 2H). MS m/z: 497.2 [M+H]⁺.

### 3-Methyl-7-(1-(3-(trifluoromethyl)pyrazin-2-yl)piperidin-4-yl)-5-((3-(trifluoromethyl)pyridin-2-yl)methyl)pyrido[2,3-b]pyrazin-6(5H)-one (156)

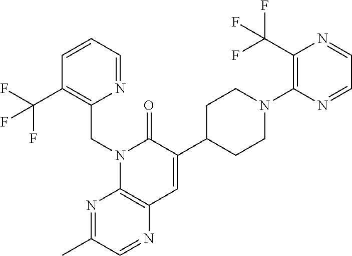 [SMILES: Cc1cnc2cc(C3CCN(c4nccnc4C(F)(F)F)CC3)c(=O)n(Cc3ncccc3C(F)(F)F)c2n1]

Step 1: 3-methyl-7-(1-(3-(trifluoromethyl)pyrazin-2-yl)piperidin-4-yl)-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (50 mg, 124 μmol, 1.0 equiv) an 2-chloro-3-(trifluoromethyl)pyrazine (22.5 mg, 124 μmol, 1.0 equiv) as the starting materials to give 3-methyl-7-(1-(3-methylpyrazin-2-yl)piperidin-4-yl)-5-((3-(trifluoromethoxy)pyridin-2-yl)methyl)pyrido[2,3-b]pyrazin-6(5H)-one (29.4 mg, 43.2%) as a white solid. ¹H NMR (400 MHz, Chloroform-d) δ 8.49-8.43 (m, 1H), 8.36-8.28 (m, 2H), 8.13 (d, J=2.3 Hz, 1H), 8.05-7.98 (m, 1H), 7.85 (d, J=0.9 Hz, 1H), 7.31-7.28 (m, 1H), 6.05 (s, 2H), 4.01 (d, J=13.0 Hz, 2H), 3.24 (ddd, J=12.1, 8.9, 3.4 Hz, 1H), 3.13 (dd, J=13.6, 11.5 Hz, 2H), 2.53 (s, 3H), 2.12 (d, J=12.9 Hz, 2H), 1.79 (qd, J=12.5, 3.7 Hz, 2H). MS m/z: 550.2 [M+H]⁺.

### 3-Methyl-5-((3-(trifluoromethyl)pyridin-2-yl)methyl)-7-(1-(2-(trifluoromethyl)pyridin-3-yl)piperidin-4-yl)pyrido[2,3-b]pyrazin-6(5H)-one (157)

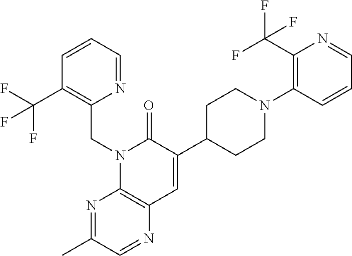 [SMILES: Cc1cnc2cc(C3CCN(c4cccnc4C(F)(F)F)CC3)c(=O)n(Cc3ncccc3C(F)(F)F)c2n1]

Step 1: tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate: Followed the General Procedure E using tert-butyl 4-(6-oxo-5,6-dihydropyrido[2,3-b]pyrazin-7-yl)piperidine-1-carboxylate (200 mg, 0.606 mmol, 1 equiv) and 2-(bromomethyl)-3-(trifluoromethyl)pyridine hydrobromide (186 mg, 0.581 mmol, 1 equiv) as the starting materials to give tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (150 mg, 49%) as a yellow solid. MS m/z: 504 [M+H]⁺

Step 2: 3-methyl-7-(piperidin-4-yl)-5-((3-(trifluoromethyl)pyridin-2-yl)methyl)pyrido[2,3-b]pyrazin-6(5H)-one: Followed the General Procedure F using tert-butyl 4-(3-methyl-6-oxo-5-((3-(trifluoromethyl)pyridin-2-yl)methyl)-5,6-dihydropyrido[2,3-b]pyrazin-7-yl)piperidine-1-carboxylate (150 mg, 0.298 mmol, 1 equiv) as the starting material to give the crude product 3-methyl-7-(piperidin-4-yl)-5-((3-(trifluoromethyl)pyridin-2-yl)methyl)pyrido[2,3-b]pyrazin-6(5H)-one (60 mg) as a white solid. MS m/z: 404 [M+H]⁺.

Step 3: 3-methyl-5-((3-(trifluoromethyl)pyridin-2-yl)methyl)-7-(1-(2-(trifluoromethyl)pyridin-3-yl)piperidin-4-yl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (60 mg, 0.149 mmol, 1 equiv) and 3-bromo-2-(trifluoromethyl)pyridine (40.3 mg, 0.179 mmol, 1.2 equiv) as the starting material to give the product 3-methyl-5-((3-(trifluoromethyl)pyridin-2-yl)methyl)-7-(1-(2-(trifluoromethyl)pyridin-3-yl)piperidin-4-yl)pyrido[2,3-b]pyrazin-6(5H)-one (22.1 mg, 26.7%) as a white solid. ¹H NMR (400 MHz, Chloroform-d) δ 8.47-8.40 (m, 2H), 8.35 (s, 1H), 7.98 (d, J=7.8 Hz, 1H), 7.87 (d, J=1.0 Hz, 1H), 7.71 (d, J=8.2 Hz, 1H), 7.50-7.42 (m, 1H), 7.23 (d, J=4.9 Hz, 1H), 6.04 (s, 2H), 3.30-3.10 (m, 3H), 2.93 (t, J=11.4 Hz, 2H), 2.52 (s, 3H), 2.10 (d, J=12.5 Hz, 2H), 1.87 (td, J=12.3, 3.8 Hz, 2H), 1.26 (s, 1H). MS m/z: 549.20 [M+H]⁺.

### 3-Methyl-7-((1r,4r)-4-(thiazol-2-yl)cyclohexyl)-5-((3-(trifluoromethyl)pyridin-2-yl)methyl)pyrido[2,3-b]pyrazin-6(5H)-one (158)

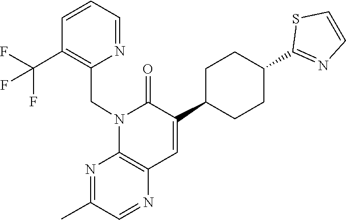 [SMILES: Cc1cnc2cc([C@H]3CC[C@H](c4nccs4)CC3)c(=O)n(Cc3ncccc3C(F)(F)F)c2n1]

Step 1: ethyl 2-(4-(thiazol-2-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (500 mg, 1.70 mmol, 1 equiv.) and 2-bromothiazole (334 mg, 2.04 mmol, 1.2 equiv.) as the starting materials to give ethyl 2-(4-(thiazol-2-yl)cyclohex-3-en-1-yl)acetate (420 mg, 98%) as a colorless oil. MS m/z: 252 [M+H]⁺.

Step 2: ethyl 2-(4-(thiazol-2-yl)cyclohexyl)acetate: Followed the General Procedure AR using ethyl 2-(4-(thiazol-2-yl)cyclohex-3-en-1-yl)acetate (420 mg, 1.67 mmol, 1 equiv.) as the starting material to give the crude product ethyl 2-(4-(thiazol-2-yl)cyclohexyl)acetate (390 mg) as a colorless oil. MS m/z: 254 [M+H]⁺.

Step 3: 3-methyl-7-((1r,4r)-4-(thiazol-2-yl)cyclohexyl)pyrido[2,3-b]pyrazin-6(5H)-one: Followed the General Procedure D using ethyl 2-(4-(thiazol-2-yl)cyclohexyl)acetate (390 mg, 1.54 mmol) and 3-amino-5-methylpyrazine-2-carbaldehyde (253 mg, 1.84 mmol, 1.2 equiv.) as the starting materials to give 3-methyl-7-((1r,4r)-4-(thiazol-2-yl)cyclohexyl)pyrido[2,3-b]pyrazin-6(5H)-one (40 mg, 8%) as a white solid. MS m/z: 327 [M+H]⁺.

Step 4: 3-methyl-7-((1r,4r)-4-(thiazol-2-yl)cyclohexyl)-5-((3-(trifluoromethyl)pyridin-2-yl)methyl)pyrido[2,3-b]pyrazin-6(5H)-one: Followed the General Procedure E using 3-methyl-7-((1r,4r)-4-(thiazol-2-yl)cyclohexyl)pyrido[2,3-b]pyrazin-6(5H)-one (40 mg, 75.9 μmol, 1 equiv.) and 2-(bromomethyl)-3-(trifluoromethyl)pyridine hydrobromide (17.7 mg, 91 μmol, 1.2 equiv.) as the starting materials to give 3-methyl-7-((1r,4r)-4-(thiazol-2-yl)cyclohexyl)-5-((3-(trifluoromethyl)pyridin-2-yl)methyl)pyrido[2,3-b]pyrazin-6(5H)-one (11.8 mg, 32%) as a white solid. MS m/z: 486 [M+H]⁺. MS m/z: 544 [M+H]⁺. ¹H NMR (400 MHz, Chloroform-d) δ 8.42 (d, J=4.4 Hz 1H), 8.33 (s, 1H), 7.97 (d, J=7.6 Hz, 1H), 7.80 (d, J=0.9 Hz, 1H), 7.73 (d, J=3.4 Hz, 1H), 7.25-7.20 (m, 2H), 6.03 (s, 2H), 3.26-3.15 (m, 1H), 3.15-3.00 (m, 1H), 2.51 (s, 3H), 2.37 (d, J=12.8 Hz, 2H), 2.25-2.16 (m, 2H), 1.84-1.74 (m, 2H), 1.62-1.58 (m, 2H).

### 5-((4-(Difluoromethyl)-3-oxo-3,4-dihydropyrazin-2-yl)methyl)-7-(1-(2-fluoro-6-methylphenyl)piperidin-4-yl)pyrido[2,3-b]pyrazin-6(5H)-one (159)

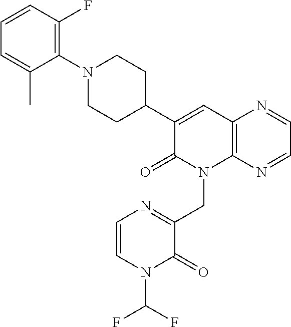 [SMILES: Cc1cccc(F)c1N1CCC(c2cc3nccnc3n(Cc3nccn(C(F)F)c3=O)c2=O)CC1]

Step 1: 5-((4-(difluoromethyl)-3-oxo-3,4-dihydropyrazin-2-yl)methyl)-7-(1-(2-fluoro-6-methylphenyl)piperidin-4-yl)pyrido[2,3-b]pyrazin-6(5H)-one: Followed the General Procedure AU using 7-(1-(2-fluoro-6-methylphenyl)piperidin-4-yl)-5-((3-hydroxypyrazin-2-yl)methyl)pyrido[2,3-b]pyrazin-6(5H)-one (80 mg, 0.179 mmol, 1 equiv) and methyl 2-chloro-2,2-difluoroacetate (51.5 mg, 0.358 mmol, 2 equiv) as the starting materials to give 5-((4-(difluoromethyl)-3-oxo-3,4-dihydropyrazin-2-yl)methyl)-7-(1-(2-fluoro-6-methylphenyl)piperidin-4-yl)pyrido[2,3-b]pyrazin-6(5H)-one (16.6 mg, 18.6%) as an off-white solid. ¹H NMR (400 MHz, Chloroform-d) δ 8.48 (d, J=2.3 Hz, 1H), 8.38 (d, J=2.3 Hz, 1H), 7.88 (s, 1H), 7.65 (t, J=59.8 Hz, 1H), 7.19-7.09 (m, 2H), 7.03-6.95 (m, 2H), 6.92-6.83 (m, 1H), 5.85 (s, 2H), 3.44-3.29 (m, 2H), 3.25-3.02 (m, 3H), 2.38 (s, 3H), 2.11-1.98 (m, 2H), 1.97-1.78 (m, 2H). MS m/z: 497.1 [M+H]⁺.

### (S)-5-(6,7-Dihydro-5H-cyclopenta[b]pyrazin-5-yl)-7-(1-(2-fluoro-6-methylphenyl)piperidin-4-yl)pyrido[2,3-b]pyrazin-6(5H)-one (160A); (R)-5-(6,7-Dihydro-5H-cyclopenta[b]pyrazin-5-yl)-7-(1-(2-fluoro-6-methylphenyl)piperidin-4-yl)pyrido[2,3-b]pyrazin-6(5H)-one (160B)

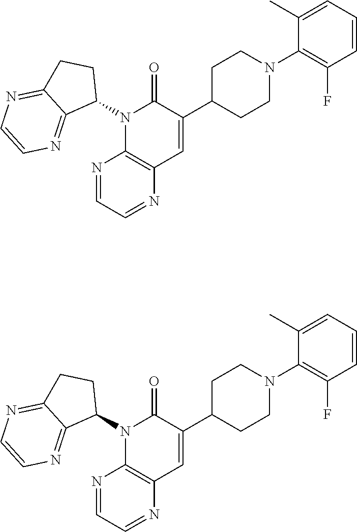 [SMILES: Cc1cccc(F)c1N1CCC(c2cc3nccnc3n([C@H]3CCc4nccnc43)c2=O)CC1 ; Cc1cccc(F)c1N1CCC(c2cc3nccnc3n([C@@H]3CCc4nccnc43)c2=O)CC1]

Step 1: 6,7-dihydro-5H-cyclopenta[b]pyrazine: To a stirred solution of 2-hydroxycyclopent-2-en-1-one (1 g, 10.2 mmol, 1 equiv) and ethane-1,2-diamine (674 mg, 11.2 mmol, 1.1 equiv) in ethylene glycol (10 mL) was added NaOH (1.01 g, 25.5 mmol, 2.5 equiv) at room temperature under air atmosphere. The resulting mixture was stirred for overnight at 150° C. under air atmosphere. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:1) to afford 6,7-dihydro-5H-cyclopenta[b]pyrazine (400 mg, 32%) as a yellow oil. MS m/z: 121 [M+H]⁺.

Step 2: 5-bromo-6,7-dihydro-5H-cyclopenta[b]pyrazine: Followed the General Procedure AF using 6,7-dihydro-5H-cyclopenta[b]pyrazine (500 mg, 4.16 mmol, 1 equiv) and NBS (370 mg, 2.08 mmol, 0.5 equiv) as the starting materials to give 5-bromo-6,7-dihydro-5H-cyclopenta[b]pyrazine (100 mg, 12%) as a colorless oil. MS m/z: 199 [M+H]⁺.

Step 3: tert-butyl 4-(5-(6,7-dihydro-5H-cyclopenta[b]pyrazin-5-yl)-6-oxo-5,6-dihydropyrido[2,3-b]pyrazin-7-yl)piperidine-1-carboxylate: Followed the General Procedure E using tert-butyl 4-(6-oxo-5,6-dihydropyrido[2,3-b]pyrazin-7-yl)piperidine-1-carboxylate (100 mg, 0.303 mmol, 1 equiv) and 5-bromo-6,7-dihydro-5H-cyclopenta[b]pyrazine (60 mg, 0.303 mmol, 1 equiv) as the starting materials to give tert-butyl 4-(5-(6,7-dihydro-5H-cyclopenta[b]pyrazin-5-yl)-6-oxo-5,6-dihydropyrido[2,3-b]pyrazin-7-yl)piperidine-1-carboxylate (80 mg, 59%) as a white solid. MS m/z: 449 [M+H]⁺.

Step 4: 5-(6,7-dihydro-5H-cyclopenta[b]pyrazin-5-yl)-7-(piperidin-4-yl)pyrido[2,3-b]pyrazin-6(5H)-one: Followed the General Procedure F using tert-butyl 4-(5-(6,7-dihydro-5H-cyclopenta[b]pyrazin-5-yl)-6-oxo-5,6-dihydropyrido[2,3-b]pyrazin-7-yl)piperidine-1-carboxylate (80 mg, 0.178 mmol, 1 equiv) as the starting material to give the crude product 5-(6,7-dihydro-5H-cyclopenta[b]pyrazin-5-yl)-7-(piperidin-4-yl)pyrido[2,3-b]pyrazin-6(5H)-one (50 mg) as a white solid. MS m/z: 349 [M+H]⁺.

Step 5: 5-(6,7-dihydro-5H-cyclopenta[b]pyrazin-5-yl)-7-(1-(2-fluoro-6-methylphenyl)piperidin-4-yl)pyrido[2,3-b]pyrazin-6(5H)-one: Followed the General Procedure G using 5-(6,7-dihydro-5H-cyclopenta[b]pyrazin-5-yl)-7-(piperidin-4-yl)pyrido[2,3-b]pyrazin-6(5H)-one (50 mg, 0.144 mmol, 1 equiv) and 2-bromo-1-fluoro-3-methylbenzene (54 mg, 0.288 mmol, 2 equiv) as the starting materials to give the crude product 5-(6,7-dihydro-5H-cyclopenta[b]pyrazin-5-yl)-7-(1-(2-fluoro-6-methylphenyl)piperidin-4-yl)pyrido[2,3-b]pyrazin-6(5H)-one (20 mg) as a white solid. The crude product was purified by Chiral-HPLC with the following conditions (Flow rate: 1.67 ml/min; Gradient: isocratic; Injection Volume: 10 mL) to afford (S)-5-(6,7-dihydro-5H-cyclopenta[b]pyrazin-5-yl)-7-(1-(2-fluoro-6-methylphenyl)piperidin-4-yl)pyrido[2,3-b]pyrazin-6(5H)-one (5.3 mg, 18.3%) as a white solid and (R)-5-(6,7-dihydro-5H-cyclopenta[b]pyrazin-5-yl)-7-(1-(2-fluoro-6-methylphenyl)piperidin-4-yl)pyrido[2,3-b]pyrazin-6(5H)-one (5 mg, 18%) as a white solid.

160A: (S)-5-(6,7-dihydro-5H-cyclopenta[b]pyrazin-5-yl)-7-(1-(2-fluoro-6-methylphenyl)piperidin-4-yl)pyrido[2,3-b]pyrazin-6(5H)-one. ¹H NMR (400 MHz, Chloroform-d) δ 8.58-8.49 (m, 1H), 8.39-7.95 (m, 3H), 7.86-7.79 (m, 1H), 7.39-7.29 (m, 1H), 7.16-6.80 (m, 3H), 3.62-2.47 (m, 9H), 2.43-2.30 (m, 3H), 2.11-1.73 (m, 4H). MS m/z: 457.2 [M+H]⁺

160B: (R)-5-(6,7-dihydro-5H-cyclopenta[b]pyrazin-5-yl)-7-(1-(2-fluoro-6-methylphenyl)piperidin-4-yl)pyrido[2,3-b]pyrazin-6(5H)-one. ¹H NMR (400 MHz, Chloroform-d) δ 8.58-8.47 (m, 1H), 8.41-7.88 (m, 3H), 7.87-7.74 (m, 1H), 7.40-7.31 (m, 1H), 7.17-6.77 (m, 3H), 3.58-2.57 (m, 9H), 2.38-2.29 (m, 3H), 2.11-1.70 (m, 4H). MS m/z: 457.2 [M+H]⁺

### 5-((3-(Difluoromethoxy)pyrazin-2-yl)methyl)-7-(4-(2-fluoro-6-methylphenyl)piperazin-1-yl)-3-methylpyrido[2,3-b]pyrazin-6(5H)-one (161A); 5-((4-(Difluoromethyl)-3-oxo-3,4-dihydropyrazin-2-yl)methyl)-7-(4-(2-fluoro-6-methylphenyl)piperazin-1-yl)-3-methylpyrido[2,3-b]pyrazin-6(5H)-one (161B)

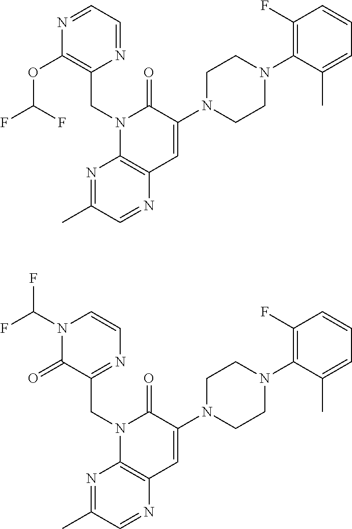 [SMILES: Cc1cnc2cc(N3CCN(c4c(C)cccc4F)CC3)c(=O)n(Cc3nccnc3OC(F)F)c2n1 ; Cc1cnc2cc(N3CCN(c4c(C)cccc4F)CC3)c(=O)n(Cc3nccn(C(F)F)c3=O)c2n1]

Step 1: ethyl 2-(4-(2-fluoro-6-methylphenyl)piperazin-1-yl)acetate: Followed the General Procedure G using ethyl 2-(piperazin-1-yl)acetate (120 mg, 0.697 mmol, 1 equiv) and 2-bromo-1-fluoro-3-methylbenzene (144 mg, 0.766 mmol, 1.1 equiv) as the starting materials to give ethyl 2-(4-(2-fluoro-6-methylphenyl)piperazin-1-yl)acetate (100 mg, 58%) as a yellow solid. MS m/z: 281 [M+H]⁺.

Step 2: 7-(4-(2-fluoro-6-methylphenyl)piperazin-1-yl)-3-methylpyrido[2,3-b]pyrazin-6(5H)-one: Followed the General Procedure D using ethyl 2-(4-(2-fluoro-6-methylphenyl)piperazin-1-yl)acetate (100 mg, 0.354 mmol, 1 equiv) and 3-amino-5-methylpyrazine-2-carbaldehyde (58.2 mg, 0.424 mmol, 1.2 equiv) as the starting materials to give 7-(4-(2-fluoro-6-methylphenyl)piperazin-1-yl)-3-methylpyrido[2,3-b]pyrazin-6(5H)-one (60 mg, 36%) as a yellow solid. MS m/z: 354 [M+H]⁺.

Step 3: 7-(4-(2-fluoro-6-methylphenyl)piperazin-1-yl)-3-methyl-5-((3-((tetrahydro-2H-pyran-2-yl)oxy)pyrazin-2-yl)methyl)pyrido[2,3-b]pyrazin-6(5H)-one: Followed the General Procedure E using 7-(4-(2-fluoro-6-methylphenyl)piperazin-1-yl)-3-methylpyrido[2,3-b]pyrazin-6(5H)-one (60 mg, 0.169 mmol, 1 equiv) and 2-(bromomethyl)-3-((tetrahydro-2H-pyran-2-yl)oxy)pyrazine (50 mg, 0.184 mmol, 1.1 equiv) as the starting materials to give 7-(4-(2-fluoro-6-methylphenyl)piperazin-1-yl)-3-methyl-5-((3-((tetrahydro-2H-pyran-2-yl)oxy)pyrazin-2-yl)methyl)pyrido[2,3-b]pyrazin-6(5H)-one (45 mg, 52%) as a yellow solid. MS m/z: 546 [M+H]⁺.

Step 4: 7-(4-(2-fluoro-6-methylphenyl)piperazin-1-yl)-5-((3-hydroxypyrazin-2-yl)methyl)-3-methylpyrido[2,3-b]pyrazin-6(5H)-one: Followed the General Procedure F using 7-(4-(2-fluoro-6-methylphenyl)piperazin-1-yl)-3-methyl-5-((3-((tetrahydro-2H-pyran-2-yl)oxy)pyrazin-2-yl)methyl)pyrido[2,3-b]pyrazin-6(5H)-one (45 mg, 0.083 mmol, 1 equiv) as the starting material to give the crude product 7-(4-(2-fluoro-6-methylphenyl)piperazin-1-yl)-5-((3-hydroxypyrazin-2-yl)methyl)-3-methylpyrido[2,3-b]pyrazin-6(5H)-one (40 mg) as a white solid. MS m/z: 462 [M+H]⁺.

Step 5: 5-((3-(difluoromethoxy)pyrazin-2-yl)methyl)-7-(4-(2-fluoro-6-methylphenyl)piperazin-1-yl)-3-methylpyrido[2,3-b]pyrazin-6(5H)-one: Followed the General Procedure AU using 7-(4-(2-fluoro-6-methylphenyl)piperazin-1-yl)-5-((3-hydroxypyrazin-2-yl)methyl)-3-methylpyrido[2,3-b]pyrazin-6(5H)-one (40 mg, 0.087 mmol, 1 equiv) and methyl 2-chloro-2,2-difluoroacetate (15 mg, 0.104 mmol, 1.2 equiv) as the starting materials to give 5-((3-(difluoromethoxy)pyrazin-2-yl)methyl)-7-(4-(2-fluoro-6-methylphenyl)piperazin-1-yl)-3-methylpyrido[2,3-b]pyrazin-6(5H)-one (10 mg, 22.6%) and 5-((4-(difluoromethyl)-3-oxo-3,4-dihydropyrazin-2-yl)methyl)-7-(4-(2-fluoro-6-methylphenyl)piperazin-1-yl)-3-methylpyrido[2,3-b]pyrazin-6(5H)-one (8.7 mg, 19.6%) as a yellow solid.

161A: 5-((3-(difluoromethoxy)pyrazin-2-yl)methyl)-7-(4-(2-fluoro-6-methylphenyl)piperazin-1-yl)-3-methylpyrido[2,3-b]pyrazin-6(5H)-one. ¹H NMR (400 MHz, Chloroform-d) δ 8.26 (s, 1H), 8.11 (d, J=2.8 Hz, 1H), 7.97 (d, J=2.8 Hz, 1H), 7.71-7.32 (m, 1H), 7.22 (s, 1H), 6.99 (d, J=12.2 Hz, 2H), 6.91-6.82 (m, 1H), 5.92 (s, 2H), 3.91-3.01 (s, 8H), 2.50 (d, J=3.8 Hz, 3H), 2.37 (s, 3H). MS m/z: 512.20 [M+H]⁺.

161B: 5-((4-(difluoromethyl)-3-oxo-3,4-dihydropyrazin-2-yl)methyl)-7-(4-(2-fluoro-6-methylphenyl)piperazin-1-yl)-3-methylpyrido[2,3-b]pyrazin-6(5H)-one. ¹H NMR (400 MHz, Chloroform-d) δ 8.26 (s, 1H), 7.97 (d, J=2.8 Hz, 1H), 6.99 (d, J=12.2 Hz, 3H), 6.91-6.82 (m, 3H), 5.92 (s, 2H), 3.91-3.01 (s, 8H), 2.50 (d, J=3.8 Hz, 3H), 2.37 (s, 3H). MS m/z: 512.15 [M+H]⁺.

### 7-(1-(4-(Difluoromethyl)pyridin-3-yl)piperidin-4-yl)-3-methyl-5-((3-(trifluoromethyl)pyridin-2-yl)methyl)pyrido[2,3-b]pyrazin-6(5H)-one (162)

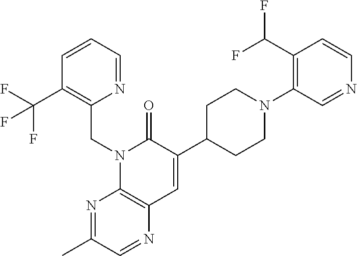 [SMILES: Cc1cnc2cc(C3CCN(c4cnccc4C(F)F)CC3)c(=O)n(Cc3ncccc3C(F)(F)F)c2n1]

Step 1: 7-(1-(4-(difluoromethyl)pyridin-3-yl)piperidin-4-yl)-3-methyl-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (50 mg, 0.124 mmol, 1 equiv) and 3-bromo-4-(difluoromethyl)pyridine (30.94 mg, 0.149 mmol, 1.2 equiv) as the starting materials to give 7-(1-(4-(difluoromethyl)pyridin-3-yl)piperidin-4-yl)-3-methyl-5-((3-(trifluoromethyl)pyridin-2-yl)methyl)pyrido[2,3-b]pyrazin-6(5H)-one (17.9 mg, 27.0%) as a white solid. ¹H NMR (400 MHz, Chloroform-d) δ 8.46-8.40 (m, 2H), 8.34 (s, 1H), 8.00-7.92 (m, 2H), 7.84 (d, J=1.0 Hz, 1H), 7.25-7.20 (m, 1H), 7.10-7.05 (m, 1H), 6.86 (s, 1H), 6.03 (s, 2H), 3.62 (d, J=12.5 Hz, 2H), 3.26-3.11 (m, 3H), 2.52 (s, 3H), 2.14 (d, J=12.6 Hz, 2H), 1.93-1.79 (m, 2H). MS m/z: 531.1 [M+H]⁺.

### 3-Methyl-7-((1r,4r)-4-(5-methylthiazol-4-yl)cyclohexyl)-5-((3-(trifluoromethyl)pyridin-2-yl)methyl)pyrido[2,3-b]pyrazin-6(5H)-one (163)

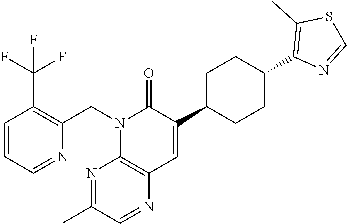 [SMILES: Cc1cnc2cc([C@H]3CC[C@H](c4ncsc4C)CC3)c(=O)n(Cc3ncccc3C(F)(F)F)c2n1]

Step 1: 3-methyl-7-((1r,4r)-4-(5-methylthiazol-4-yl)cyclohexyl)-5-((3-(trifluoromethyl)pyridin-2-yl)methyl)pyrido[2,3-b]pyrazin-6(5H)-one: Followed the General Procedure E using 3-methyl-7-((1r,4r)-4-(5-methylthiazol-4-yl)cyclohexyl)pyrido[2,3-b]pyrazin-6(5H)-one (30 mg, 0.089 mmol, 1 equiv.) and 2-(bromomethyl)-3-(trifluoromethyl)pyridine hydrobromide (42.3 mg, 0.132 mmol, 1.5 equiv.) as the starting materials to give 3-methyl-7-((1r,4r)-4-(5-methylthiazol-4-yl)cyclohexyl)-5-((3-(trifluoromethyl)pyridin-2-yl)methyl)pyrido[2,3-b]pyrazin-6(5H)-one (11.3 mg, 39.9%) as a white solid. ¹H NMR (400 MHz, Chloroform-d) δ 8.60 (s, 1H), 8.40 (d, J=4.4 Hz, 1H), 8.31 (s, 1H), 7.99-7.93 (m, 2H), 7.24-7.17 (m, 1H), 6.02 (s, 2H), 3.28-3.11 (m, 2H), 2.50 (s, 3H), 2.43 (s, 3H), 2.22-2.16 (m, 2H), 2.12-2.05 (m, 2H), 1.92-1.87 (m, 4H). MS m/z: 500.10 [M+H]⁺.

### 3-Methyl-7-((1r,4r)-4-(4-methylthiazol-5-yl)cyclohexyl)-5-((3-(trifluoromethyl)pyridin-2-yl)methyl)pyrido[2,3-b]pyrazin-6(5H)-one (164)

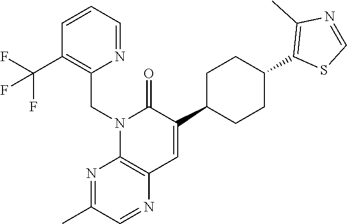 [SMILES: Cc1cnc2cc([C@H]3CC[C@H](c4scnc4C)CC3)c(=O)n(Cc3ncccc3C(F)(F)F)c2n1]

Step 1: ethyl 2-(4-(4-methylthiazol-5-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (500 mg, 1.70 mmol, 1.0 equiv) and 5-bromo-4-methylthiazole (301 mg, 1.70 mmol, 1.0 equiv.) as the starting materials to give ethyl 2-(4-(4-methylthiazol-5-yl)cyclohex-3-en-1-yl)acetate (400 mg, 88%) as a white solid. MS m/z: 266 [M+H]⁺.

Step 2: ethyl 2-(4-(4-methylthiazol-5-yl)cyclohexyl)acetate: Followed the General Procedure AR using ethyl 2-(4-(4-methylthiazol-5-yl)cyclohex-3-en-1-yl)acetate (400 mg) as the starting material to give the crude product ethyl 2-(4-(4-methylthiazol-5-yl)cyclohexyl)acetate (330 mg). MS m/z: 268 [M+H]⁺.

Step 3: 3-methyl-7-(4-(4-methylthiazol-5-yl)cyclohexyl)pyrido[2,3-b]pyrazin-6(5H)-one: Followed the General Procedure D using ethyl 2-(4-(4-methylthiazol-5-yl)cyclohexyl)acetate (330 mg, 1.23 mmol, 1.0 equiv.) and 3-amino-5-methylpyrazine-2-carbaldehyde (169 mg, 1.23 mmol, 1.0 equiv.) as the starting material to give the crude product 3-methyl-7-(4-(4-methylthiazol-5-yl)cyclohexyl)pyrido[2,3-b]pyrazin-6(5H)-one (220 mg). The crude product was purified by Prep-HPLC with the following conditions (Column: XBridge Shield RP18 OBD Column, 30*150 mm, 5 m; Mobile Phase A: Water (10 mmol/L NH₄HCO₃), Mobile Phase B: ACN; Flow rate: 60 mL/min mL/min; Gradient: 28% B to 32% B in 10 min; Wave Length: 254 nm/220 nm nm; RT1 (min): 9.6) to afford 3-methyl-7-((1r,4r)-4-(4-methylthiazol-5-yl)cyclohexyl)pyrido[2,3-b]pyrazin-6(5H)-one (55 mg, 25%) as a white solid. MS m/z: 341 [M+H]⁺.

Step 4: 3-methyl-7-((1r,4r)-4-(4-methylthiazol-5-yl)cyclohexyl)-5-((3-(trifluoromethyl)pyridin-2-yl)methyl)pyrido[2,3-b]pyrazin-6(5H)-one: Followed the General Procedure E using 3-methyl-7-((1r,4r)-4-(4-methylthiazol-5-yl)cyclohexyl)pyrido[2,3-b]pyrazin-6(5H)-one (55 mg, 161 μmol, 1.0 equiv) and 2-(bromomethyl)-3-(trifluoromethyl)pyridine (38.6 mg, 161 μmol, 1.0 equiv) as the starting materials to give 3-methyl-7-((1r,4r)-4-(4-methylthiazol-5-yl)cyclohexyl)-5-((3-(trifluoromethyl)pyridin-2-yl)methyl)pyrido[2,3-b]pyrazin-6(5H)-one (17.4 mg, 21.5%) as a white solid. ¹H NMR (300 MHz, Chloroform-d) δ 8.70 (s, 1H), 8.45 (d, J=4.8 Hz, 1H), 8.36 (s, 1H), 7.99 (d, J=7.9 Hz, 1H), 7.81 (s, 1H), 7.25 (d, J=6.6 Hz, 1H), 6.05 (s, 2H), 3.20-2.90 (m, 2H), 2.54 (s, 3H), 2.51 (s, 3H), 2.24-2.11 (m, 4H), 1.69-1.58 (m, 4H). MS m/z: 500.1 [M+H]⁺.

### 3-Methyl-7-(1-(4-(trifluoromethyl)pyridazin-3-yl)piperidin-4-yl)-5-((3-(trifluoromethyl) pyridin-2-yl)methyl)pyrido[2,3-b]pyrazin-6(5H)-one (165)

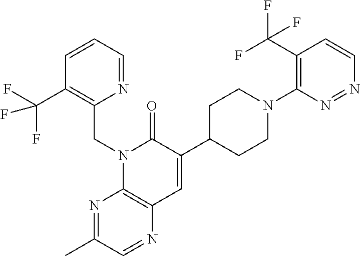 [SMILES: Cc1cnc2cc(C3CCN(c4nnccc4C(F)(F)F)CC3)c(=O)n(Cc3ncccc3C(F)(F)F)c2n1]

Step 1: 3-methyl-7-(1-(4-(trifluoromethyl)pyridazin-3-yl)piperidin-4-yl)-5-((3-(trifluoromethyl) pyridine-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (65 mg, 0.161 mmol, 1 equiv) and 3-chloro-4-(trifluoromethyl)pyridazine (35.2 mg, 0.193 mmol, 1.2 equiv) as the starting materials to give 3-methyl-7-(1-(4-(trifluoromethyl)pyridazin-3-yl)piperidin-4-yl)-5-((3-(trifluoromethyl) pyridin-2-yl)methyl)pyrido[2,3-b]pyrazin-6(5H)-one (20.6 mg, 22.4%) as a light yellow solid. ¹H NMR (400 MHz, Chloroform-d) δ 8.95 (d, J=4.9 Hz, 1H), 8.43-8.42 (m, 1H), 8.34 (s, 1H), 7.99-7.96 (m, 1H), 7.83 (s, 1H), 7.54 (d, J=5.0 Hz, 1H), 7.24-7.22 (m, 1H), 6.03 (s, 2H), 4.02 (d, J=12.9 Hz, 2H), 3.28 (t, J=12.1 Hz, 3H), 2.52 (s, 3H), 2.15 (d, J=12.3 Hz, 2H), 1.90-1.80 (m, 2H). MS m/z: 550.15 [M+H]⁺.

### 3-Methyl-7-((1r,4r)-4-(5-methylthiazol-4-yl)cyclohexyl)-5-((3-(trifluoromethoxy)pyridin-2-yl)methyl)pyrido[2,3-b]pyrazin-6(5H)-one (166)

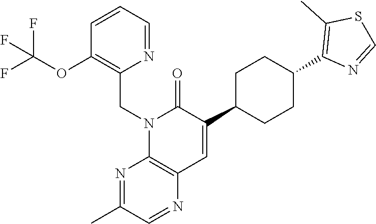 [SMILES: Cc1cnc2cc([C@H]3CC[C@H](c4ncsc4C)CC3)c(=O)n(Cc3ncccc3OC(F)(F)F)c2n1]

Step 1: ethyl 2-(4-(5-methylthiazol-4-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (200 mg, 0.680 mmol, 1 equiv.) and 4-bromo-5-methylthiazole (181 mg, 1.02 mmol, 1.5 equiv.) as the starting materials to give ethyl 2-(4-(5-methylthiazol-4-yl)cyclohex-3-en-1-yl)acetate (150 mg, 83%) as a light yellow oil. MS m/z: 266 [M+H]⁺.

Step 2: ethyl 2-(4-(5-methylthiazol-4-yl)cyclohexyl)acetate: Followed the General Procedure AR using ethyl 2-(4-(5-methylthiazol-4-yl)cyclohex-3-en-1-yl)acetate (150 mg, 0.565 mmol, 1 equiv.) as the starting material to give ethyl 2-(4-(5-methylthiazol-4-yl)cyclohexyl)acetate (130 mg, 86%) as a colorless oil. MS m/z: 268 [M+H]⁺.

Step 3: 3-methyl-7-((1r,4r)-4-(5-methylthiazol-4-yl)cyclohexyl)pyrido[2,3-b]pyrazin-6(5H)-one: Followed the General Procedure D using ethyl 2-(4-(5-methylthiazol-4-yl)cyclohexyl)acetate (130 mg, 0.486 mmol, 1 equiv.) and 3-amino-5-methylpyrazine-2-carbaldehyde (100 mg, 0.729 mmol, 1.5 equiv.) as the starting materials to give 3-methyl-7-((1r,4r)-4-(5-methylthiazol-4-yl)cyclohexyl)pyrido[2,3-b]pyrazin-6(5H)-one (20 mg, 12.1%) as an off-white solid. MS m/z: 341 [M+H]⁺.

Step 4: 3-methyl-7-((1r,4r)-4-(5-methylthiazol-4-yl)cyclohexyl)-5-((3-(trifluoromethoxy)pyridin-2-yl)methyl)pyrido[2,3-b]pyrazin-6(5H)-one: Followed the General Procedure E using 3-methyl-7-((1r,4r)-4-(5-methylthiazol-4-yl)cyclohexyl)pyrido[2,3-b]pyrazin-6(5H)-one (20 mg, 0.059 mmol, 1 equiv.) and 2-(chloromethyl)-3-(trifluoromethoxy)pyridine (18.7 mg, 0.088 mmol, 1.5 equiv.) as the starting materials to give 3-methyl-7-((1r,4r)-4-(5-methylthiazol-4-yl)cyclohexyl)-5-((3-(trifluoromethoxy)pyridin-2-yl)methyl)pyrido[2,3-b]pyrazin-6(5H)-one (12.1 mg, 39.9%) as a white solid. ¹H NMR (400 MHz, Chloroform-d) δ 8.58 (s, 1H), 8.31 (s, 1H), 8.24 (d, J=4.8, 1.4 Hz, 1H), 7.95 (s, 1H), 7.57 (d, J=8.4, 1.6 Hz, 1H), 7.22-7.14 (m, 1H), 5.93 (s, 2H), 3.27-3.10 (m, 2H), 2.51 (s, 3H), 2.42 (s, 3H), 2.23-2.13 (m, 2H), 2.12-2.02 (m, 2H), 1.92-1.86 (m, 4H). MS m/z: 516.15 [M+H]⁺.

### 5-((3-(Difluoromethoxy)pyrazin-2-yl)methyl)-7-(1-(2-fluoro-6-methylphenyl)piperidin-4-yl)-3-methylpyrido[2,3-b]pyrazin-6(5H)-one (167)

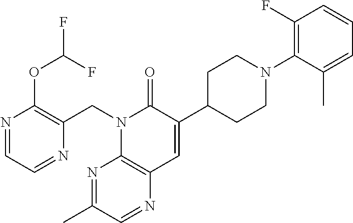 [SMILES: Cc1cnc2cc(C3CCN(c4c(C)cccc4F)CC3)c(=O)n(Cc3nccnc3OC(F)F)c2n1]

Step 1: methyl 2-(1-(2-fluoro-6-methylphenyl)piperidin-4-yl)acetate: Followed the General Procedure G using methyl 2-(piperidin-4-yl)acetate (500 mg, 3.18 mmol, 1 equiv) and 2-bromo-1-fluoro-3-methylbenzene (722 mg, 3.82 mmol, 1.2 equiv) as the starting materials to give methyl 2-(1-(2-fluoro-6-methylphenyl)piperidin-4-yl)acetate (300 mg, 47%) as a yellow solid. MS m/z: 266 [M+H]⁺.

Step 2: 7-(1-(2-fluoro-6-methylphenyl)piperidin-4-yl)-3-methylpyrido[2,3-b]pyrazin-6(5H)-one: Followed the General Procedure D using methyl 2-(1-(2-fluoro-6-methylphenyl)piperidin-4-yl)acetate (300 mg, 1.13 mmol, 1 equiv) and 3-amino-5-methylpyrazine-2-carbaldehyde (155 mg, 1.13 mmol, 1 equiv) as the starting materials to give 7-(1-(2-fluoro-6-methylphenyl)piperidin-4-yl)-3-methylpyrido[2,3-b]pyrazin-6(5H)-one (150 mg, 52%) as a yellow solid. MS m/z: 353 [M+H]⁺.

Step 3: 7-(1-(2-fluoro-6-methylphenyl)piperidin-4-yl)-3-methyl-5-((3-((tetrahydro-2H-pyran-2-yl)oxy)pyrazin-2-yl)methyl)pyrido[2,3-b]pyrazin-6(5H)-one: Followed the General Procedure E using 7-(1-(2-fluoro-6-methylphenyl)piperidin-4-yl)-3-methylpyrido[2,3-b]pyrazin-6(5H)-one (150 mg, 0.426 mmol, 1 equiv) and 2-(bromomethyl)-3-((tetrahydro-2H-pyran-2-yl)oxy)pyrazine (233 mg, 0.852 mmol, 2 equiv) as the starting materials to give 7-(1-(2-fluoro-6-methylphenyl)piperidin-4-yl)-3-methyl-5-((3-((tetrahydro-2H-pyran-2-yl)oxy)pyrazin-2-yl)methyl)pyrido[2,3-b]pyrazin-6(5H)-one (80 mg, 34%) as a white solid. MS m/z: 545 [M+H]⁺.

Step 4: 7-(1-(2-fluoro-6-methylphenyl)piperidin-4-yl)-5-((3-hydroxypyrazin-2-yl)methyl)-3-methylpyrido[2,3-b]pyrazin-6(5H)-one: Followed the General Procedure F using 7-(1-(2-fluoro-6-methylphenyl)piperidin-4-yl)-3-methyl-5-((3-((tetrahydro-2H-pyran-2-yl)oxy)pyrazin-2-yl)methyl)pyrido[2,3-b]pyrazin-6(5H)-one (80 mg, 0.147 mmol, 1 equiv) as the starting material to give the crude product 7-(1-(2-fluoro-6-methylphenyl)piperidin-4-yl)-5-((3-hydroxypyrazin-2-yl)methyl)-3-methylpyrido[2,3-b]pyrazin-6(5H)-one (80 mg) as a white solid. MS m/z: 461 [M+H]⁺.

Step 5: 5-((3-(difluoromethoxy)pyrazin-2-yl)methyl)-7-(1-(2-fluoro-6-methylphenyl)piperidin-4-yl)-3-methylpyrido[2,3-b]pyrazin-6(5H)-one: Followed the General Procedure AU using 7-(1-(2-fluoro-6-methylphenyl) piperidin-4-yl)-5-((3-hydroxypyrazin-2-yl)methyl)-3-methylpyrido[2,3-b]pyrazin-6(5H)-one (80 mg, 0.174 mmol, 1 equiv) and methyl 2-chloro-2,2-difluoroacetate (50.6 mg, 0.348 mmol, 2 equiv) as the starting materials to give 5-((3-(difluoromethoxy)pyrazin-2-yl)methyl)-7-(1-(2-fluoro-6-methylphenyl)piperidin-4-yl)-3-methylpyrido[2,3-b]pyrazin-6(5H)-one (13.4 mg, 15.1%) as a white solid. ¹H NMR (400 MHz, Chloroform-d) δ 8.37 (s, 1H), 8.10 (d, J=2.6 Hz, 1H), 8.01-7.93 (m, 2H), 7.70 (s, 1H), 7.18-7.10 (m, 1H), 7.04 (d, J=7.6 Hz, 1H), 7.00-6.93 (m, 1H), 5.91 (s, 2H), 3.63 (d, J=12.4 Hz, 2H), 3.47 (s, 2H), 3.27 (t, J=12.4 Hz, 1H), 2.70-2.65 (m, 3H), 2.54 (s, 5H), 2.11 (d, J=12.8 Hz, 2H). MS m/z: 511.15 [M+H]⁺.

### 5-((3-(Difluoromethoxy)pyrazin-2-yl)methyl)-7-((1s,4s)-4-(2-fluoro-6-methylphenyl)cyclohexyl)-3-methylpyrido[2,3-b]pyrazin-6(5H)-one (168A); 5-((3-(Difluoromethoxy)pyrazin-2-yl)methyl)-7-((1r,4r)-4-(2-fluoro-6-methylphenyl)cyclohexyl)-3-methylpyrido[2,3-b]pyrazin-6(5H)-one (168B)

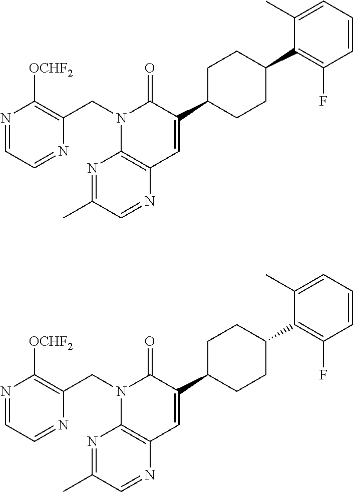 [SMILES: Cc1cnc2cc([C@H]3CC[C@@H](c4c(C)cccc4F)CC3)c(=O)n(Cc3nccnc3OC(F)F)c2n1 ; Cc1cnc2cc([C@H]3CC[C@H](c4c(C)cccc4F)CC3)c(=O)n(Cc3nccnc3OC(F)F)c2n1]

Step 1: 7-(4-(2-fluoro-6-methylphenyl)cyclohexyl)-3-methyl-5-((3-((tetrahydro-2H-pyran-2-yl)oxy)pyrazin-2-yl)methyl)pyrido[2,3-b]pyrazin-6(5H)-one: Followed the General Procedure E using 7-(4-(2-fluoro-6-methylphenyl)cyclohexyl)-3-methylpyrido[2,3-b]pyrazin-6(5H)-one (120 mg, 0.341 mmol, 1 equiv.) and 2-(bromomethyl)-3-((tetrahydro-2H-pyran-2-yl)oxy)pyrazine (139 mg, 0.513 mmol, 1.5 equiv.) as the starting materials to give 7-(4-(2-fluoro-6-methylphenyl)cyclohexyl)-3-methyl-5-((3-((tetrahydro-2H-pyran-2-yl)oxy)pyrazin-2-yl)methyl)pyrido[2,3-b]pyrazin-6(5H)-one (95 mg, 51%) as a brown solid. MS m/z: 544[M+H]⁺.

Step 2: 7-(4-(2-fluoro-6-methylphenyl)cyclohexyl)-5-((3-hydroxypyrazin-2-yl)methyl)-3-methylpyrido[2,3-b]pyrazin-6(5H)-one: A solution of 7-(4-(2-fluoro-6-methylphenyl)cyclohexyl)-3-methyl-5-((3-((tetrahydro-2H-pyran-2-yl)oxy)pyrazin-2-yl)methyl)pyrido[2,3-b]pyrazin-6(5H)-one (95 mg, 0.175 mmol, 1 equiv.) and TFA (2.00 mL) in DCM (2.00 mL) was stirred for 2 h at room temperature. After removing the solvent, the mixture was basified to pH 8 with saturated NaHCO₃ (aq.). The resulting mixture was extracted with EtOAc (3×15 mL). The combined organic layers were washed with brine (20 mL), dried over anhydrous Na₂SO₄. After filtration, the filtrate was concentrated under reduced pressure. The crude product 7-(4-(2-fluoro-6-methylphenyl)cyclohexyl)-5-((3-hydroxypyrazin-2-yl)methyl)-3-methylpyrido[2,3-b]pyrazin-6(5H)-one (70 mg) as a white solid was used for next step without further purification. MS m/z: 460 [M+H]⁺.

Step 3: 5-((3-(difluoromethoxy)pyrazin-2-yl)methyl)-7-((1s,4s)-4-(2-fluoro-6-methylphenyl)cyclohexyl)-3-methylpyrido[2,3-b]pyrazin-6(5H)-one and 5-((3-(difluoromethoxy)pyrazin-2-yl)methyl)-7-((1r,4r)-4-(2-fluoro-6-methylphenyl)cyclohexyl)-3-methylpyrido[2,3-b]pyrazin-6(5H)-one: Followed the General Procedure AU using 7-(4-(2-fluoro-6-methylphenyl)cyclohexyl)-5-((3-hydroxypyrazin-2-yl)methyl)-3-methylpyrido[2,3-b]pyrazin-6(5H)-one (70 mg, 0.153 mmol, 1.0 equiv.) and 2-chloro-2,2-difluoroacetate (33 mg, 0.229 mmol, 1.5 equiv.) as the starting materials to give 5-((3-(difluoromethoxy)pyrazin-2-yl)methyl)-7-((1s,4s)-4-(2-fluoro-6-methylphenyl)cyclohexyl)-3-methylpyrido[2,3-b]pyrazin-6(5H)-one (12.6 mg, 16.2%) as a white solid and 5-((3-(difluoromethoxy)pyrazin-2-yl)methyl)-7-((1r,4r)-4-(2-fluoro-6-methylphenyl)cyclohexyl)-3-methylpyrido[2,3-b]pyrazin-6(5H)-one (11.1 mg, 14.3%) as a white solid.

168A: 5-((3-(difluoromethoxy)pyrazin-2-yl)methyl)-7-((1s,4s)-4-(2-fluoro-6-methylphenyl)cyclohexyl)-3-methylpyrido[2,3-b]pyrazin-6(5H)-one. ¹H NMR (400 MHz, Chloroform-d) δ 8.38 (s, 1H), 8.20 (s, 1H), 8.10 (d, J=2.8 Hz, 1H), 7.97 (d, J=2.8 Hz, 1H), 7.51 (t, J=72.0 Hz, 1H), 7.05-6.97 (m, 1H), 6.94-6.88 (m, 1H), 6.86-6.80 (m, 1H), 5.90 (s, 2H), 3.48-3.43 (m, 1H), 2.94 (t, J=12.4 Hz, 1H), 2.54 (s, 3H), 2.37 (s, 3H), 2.32-2.14 (m, 4H), 1.99-1.90 (m, 2H), 1.65 (d, J=13.6 Hz, 2H). MS m/z: 510.15 [M+H]⁺.

168B: 5-((3-(difluoromethoxy)pyrazin-2-yl)methyl)-7-((1r,4r)-4-(2-fluoro-6-methylphenyl)cyclohexyl)-3-methylpyrido[2,3-b]pyrazin-6(5H)-one. ¹H NMR (400 MHz, Chloroform-d) δ 8.33 (s, 1H), 8.11 (d, J=2.8 Hz, 1H), 7.98 (d, J=2.8 Hz, 1H), 7.85 (s, 1H), 7.52 (t, J=72.0 Hz, 1H), 7.08-7.00 (m, 1H), 6.95-6.91 (m, 1H), 6.88-6.82 (m, 1H), 5.91 (s, 2H), 3.13 (t, J=12.0 Hz, 1H), 2.90 (t, J=12.4 Hz, 1H), 2.55 (s, 3H), 2.38 (s, 3H), 2.15 (t, J=11.2 Hz, 4H), 1.87-1.83 (m, 2H), 1.61-1.47 (m, 2H). MS m/z: 510.10 [M+H]⁺.

### 5-((3-Ethoxypyrazin-2-yl)methyl)-7-((1r,4r)-4-(2-fluoro-6-methylphenyl)cyclohexyl)-3-methylpyrido[2,3-b]pyrazin-6(5H)-one (169)

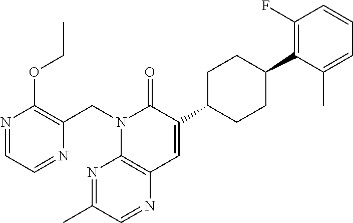 [SMILES: CCOc1nccnc1Cn1c(=O)c([C@H]2CC[C@H](c3c(C)cccc3F)CC2)cc2ncc(C)nc21]

Step 1:2-ethoxy-3-methylpyrazine: Followed the General Procedure AK using 2-chloro-3-methylpyrazine (500 mg, 3.9 mmol, 1.0 equiv) as the starting materials as the base to give 2-ethoxy-3-methylpyrazine (300 mg, 55%) as a white solid. MS m/z: 139 [M+H]⁺.

Step 2: 2-(bromomethyl)-3-ethoxypyrazine: Followed the General Procedure AF using 2-ethoxy-3-methylpyrazine (300 mg, 2.17 mmol, 1.0 equiv) as the starting material to give 2-(bromomethyl)-3-ethoxypyrazine (220 mg, 46.8%). MS m/z: 217 [M+H]⁺.

Step 3: 5-((3-ethoxypyrazin-2-yl)methyl)-7-((1r,4r)-4-(2-fluoro-6-methylphenyl)cyclohexyl)-3-methylpyrido[2,3-b]pyrazin-6(5H)-one: Followed the General Procedure E using 2-(bromomethyl)-3-ethoxypyrazine (18.4 mg, 85 μmol, 1.0 equiv) and 7-((1r,4r)-4-(2-fluoro-6-methylphenyl)cyclohexyl)-3-methylpyrido[2,3-b]pyrazin-6(5H)-one (30 mg, 85 μmol, 1.0 equiv) as the starting materials to give 5-((3-ethoxypyrazin-2-yl)methyl)-7-((1r,4r)-4-(2-fluoro-6-methylphenyl)cyclohexyl)-3-methylpyrido[2,3-b]pyrazin-6(5H)-one (10.5 mg, 7.3%) as a white solid. ¹H NMR (400 MHz, Chloroform-d) δ 8.33 (s, 1H), 7.89 (d, J=3.0 Hz, 1H), 7.84-7.77 (m, 2H), 7.03 (q, J=7.8, 7.3 Hz, 2H), 6.92 (d, J=7.6 Hz, 1H), 6.89-6.80 (m, 1H), 5.84 (s, 2H), 4.48 (d, J=6.7 Hz, 2H), 3.23-2.80 (m, 2H), 2.57-2.51 (m, 3H), 2.38 (s, 3H), 2.16 (s, 4H), 1.85 (d, J=13.0 Hz, 2H), 1.48-1.44 (m, 4H). MS m/z: 488.15 [M+H]⁺.

### 7-(1-(3-Chloropyridin-2-yl)piperidin-4-yl)-5-((3-(trifluoromethyl)pyridin-2-yl)methyl)-5,6-dihydropyrido[2,3-b]pyrazine (170)

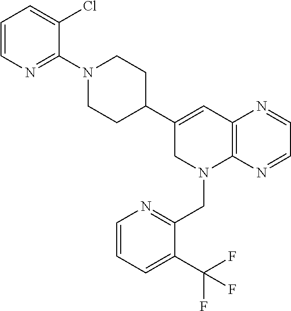 [SMILES: FC(F)(F)c1cccnc1CN1CC(C2CCN(c3ncccc3Cl)CC2)=Cc2nccnc21]

Step 1: 7-(1-(3-chloropyridin-2-yl)piperidin-4-yl)-5-((3-(trifluoromethyl)pyridin-2-yl)methyl)-5,6-dihydropyrido[2,3-b]pyrazine: Followed the General Procedure G using 7-(piperidin-4-yl)-5-((3-(trifluoromethyl)pyridin-2-yl)methyl)-5,6-dihydropyrido[2,3-b]pyrazine (75 mg, 200 μmol, 1.0 equiv) and 2-bromo-3-chloropyridine (45.8 mg, 240 μmol, 1.2 equiv) as the starting materials, t-BuOK (67.2 mg, 600 μmol, 1.0 equiv) as thre base, toluene (1 mL) as the solvent to give 7-(1-(3-chloropyridin-2-yl)piperidin-4-yl)-5-((3-(trifluoromethyl)pyridin-2-yl)methyl)-5,6-dihydropyrido[2,3-b]pyrazine (10.5 mg, 10.8%) as a white solid. ¹H NMR (400 MHz, Chloroform-d) δ 8.43 (d, J=4.9 Hz, 1H), 8.34 (s, 1H), 8.27 (d, J=5.3 Hz, 1H), 8.01-7.94 (m, 1H), 7.83 (s, 1H), 7.78 (d, J=7.3 Hz, 1H), 7.25-7.20 (m, 1H), 6.93 (t, J=6.6 Hz, 1H), 6.03 (s, 2H), 4.15 (d, J=12.7 Hz, 2H), 3.35-3.09 (m, 3H), 2.52 (s, 3H), 2.19 (d, J=12.7 Hz, 2H), 1.98-1.83 (m, 2H). MS m/z: 515.05 [M+H]⁺.

### 7-((1S,3R)-3-(2-Fluoro-6-methylphenyl)cyclopentyl)-3-methyl-5-((3-(trifluoromethyl)pyridin-2-yl)methyl)pyrido[2,3-b]pyrazin-6(5H)-one (171A); 7-((1R,3S)-3-(2-Fluoro-6-methylphenyl)cyclopentyl)-3-methyl-5-((3-(trifluoromethyl)pyridin-2-yl)methyl)pyrido[2,3-b]pyrazin-6(5H)-one (171B); 7-((1S,3S)-3-(2-Fluoro-6-methylphenyl)cyclopentyl)-3-methyl-5-((3-(trifluoromethyl)pyridin-2-yl)methyl)pyrido[2,3-b]pyrazin-6(5H)-one (171C); 7-((1R,3R)-3-(2-Fluoro-6-methylphenyl)cyclopentyl)-3-methyl-5-((3-(trifluoromethyl)pyridin-2-yl)methyl)pyrido[2,3-b]pyrazin-6(5H)-one (171D)

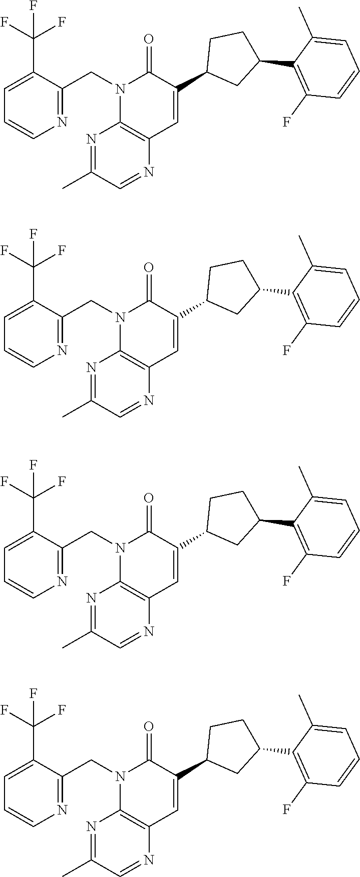 [SMILES: Cc1cnc2cc([C@H]3CC[C@@H](c4c(C)cccc4F)C3)c(=O)n(Cc3ncccc3C(F)(F)F)c2n1 ; Cc1cnc2cc([C@@H]3CC[C@H](c4c(C)cccc4F)C3)c(=O)n(Cc3ncccc3C(F)(F)F)c2n1 ; Cc1cnc2cc([C@@H]3CC[C@@H](c4c(C)cccc4F)C3)c(=O)n(Cc3ncccc3C(F)(F)F)c2n1 ; Cc1cnc2cc([C@H]3CC[C@H](c4c(C)cccc4F)C3)c(=O)n(Cc3ncccc3C(F)(F)F)c2n1]

Step 1: ethyl 2-[3-(trifluoromethanesulfonyloxy)cyclopent-2-en-1-yl]acetate: Followed the General Procedure AV using ethyl 2-(3-oxocyclopentyl)acetate (450 mg, 2.64 mmol, 1 equiv), Tf₂O (2.24 g, 7.93 mmol, 3 equiv) and lutidine (850 mg, 7.93 mmol, 3 equiv) as the starting materials to give ethyl 2-[3-(trifluoromethanesulfonyloxy)cyclopent-2-en-1-yl]acetate (680 mg, 85%) as a colorless oil. MS m/z: 303 [M+H]⁺.

Step 2: ethyl 2-[3-(2-fluoro-6-methylphenyl)cyclopent-2-en-1-yl]acetate: Followed the General Procedure AL using ethyl 2-[3-(trifluoromethanesulfonyloxy)cyclopent-2-en-1-yl]acetate (680 mg, 2.25 mmol, 1 equiv) and 2-fluoro-6-methylphenylboronic acid (415 mg, 2.7 mmol, 1.2 equiv) as the starting materials to give ethyl 2-[3-(2-fluoro-6-methylphenyl)cyclopent-2-en-1-yl]acetate (300 mg, 50%) as a colorless oil. MS m/z: 263 [M+H]⁺.

Step 3: ethyl 2-[3-(2-fluoro-6-methylphenyl)cyclopentyl]acetate: Followed the General Procedure AR using ethyl 2-[3-(2-fluoro-6-methylphenyl)cyclopent-2-en-1-yl]acetate (300 mg, 1.144 mmol, 1 equiv) as the starting materials to give ethyl 2-[3-(2-fluoro-6-methylphenyl)cyclopentyl]acetate (260 mg, 86%) as a colorless oil. MS m/z: 265 [M+H]⁺.

Step 4: 7-[3-(2-fluoro-6-methylphenyl)cyclopentyl]-3-methyl-5H-pyrido[2,3-b]pyrazin-6-one: Followed the General Procedure D using ethyl 2-[3-(2-fluoro-6-methylphenyl)cyclopentyl]acetate (260 mg, 0.984 mmol, 1 equiv) and 3-amino-5-methylpyrazine-2-carbaldehyde (161 mg, 1.18 mmol, 1.2 equiv) as the starting materials to give 7-[3-(2-fluoro-6-methylphenyl)cyclopentyl]-3-methyl-5H-pyrido[2,3-b]pyrazin-6-one (150 mg, 45%) as a yellow solid. MS m/z: 338 [M+H]⁺.

Step 5: 7-(3-(2-fluoro-6-methylphenyl)cyclopentyl)-3-methyl-5-((3-(trifluoromethyl)pyridin-2-yl)methyl)pyrido[2,3-b]pyrazin-6(5H)-one: Followed the General Procedure E using 7-[3-(2-fluoro-6-methylphenyl)cyclopentyl]-3-methyl-5H-pyrido[2,3-b]pyrazin-6-one (150 mg, 0.445 mmol, 1 equiv) and 2-(bromomethyl)-3-(trifluoromethyl)pyridine hydrobromide (142 mg, 0.445 mmol, 1 equiv) as the starting materials to afford the crude product 7-(3-(2-fluoro-6-methylphenyl)cyclopentyl)-3-methyl-5-((3-(trifluoromethyl)pyridin-2-yl)methyl)pyrido[2,3-b]pyrazin-6(5H)-one (100 mg) as a white solid.

171A: 7-((1S,3R)-3-(2-fluoro-6-methylphenyl)cyclopentyl)-3-methyl-5-((3-(trifluoromethyl)pyridin-2-yl)methyl)pyrido[2,3-b]pyrazin-6(5H)-one. The crude product (100 mg) was purified by Chiral-HPLC with the following conditions (Column: CHIRALPAK IC 2*25 cm, 5 m; Mobile Phase A: Hex (10 mM NH₃-MeOH), Mobile Phase B: IPA-HPLC; Flow rate: 20 mL/min; Gradient: isocratic 10; Wave Length: 206/210 nm; RT1 (min): 7; RT2 (min): 9; Sample Solvent: IPA-HPLC; Injection Volume: 0.3 mL; Number Of Runs: 6) to afford 7-((1S,3R)-3-(2-fluoro-6-methylphenyl)cyclopentyl)-3-methyl-5-((3-(trifluoromethyl)pyridin-2-yl)methyl)pyrido[2,3-b]pyrazin-6(5H)-one (7.3 mg, 3.2%) as a white solid. ¹H NMR (400 MHz, Chloroform-d) δ 8.44-8.38 (m, 1H), 8.31 (s, 1H), 7.99-7.92 (m, 1H), 7.88 (d, J=1.1 Hz, 1H), 7.24-7.18 (m, 1H), 7.07-6.98 (m, 1H), 6.93-6.81 (m, 2H), 6.03 (s, 2H), 3.88-3.75 (m, 1H), 3.58 (p, J=9.2 Hz, 1H), 2.50 (s, 3H), 2.49-2.40 (m, 1H), 2.37 (s, 3H), 2.35-2.28 (m, 1H), 2.17-2.02 (m, 3H), 1.98-1.88 (m, 1H). MS m/z: 497.10 [M+H]⁺.

171B: 7-((1R,3S)-3-(2-fluoro-6-methylphenyl)cyclopentyl)-3-methyl-5-((3-(trifluoromethyl)pyridin-2-yl)methyl)pyrido[2,3-b]pyrazin-6(5H)-one. The crude product (100 mg) was purified by Chiral-HPLC with the following conditions (Column: CHIRALPAK IC 2*25 cm, 5 m; Mobile Phase A: Hex (10 mM NH₃-MeOH), Mobile Phase B: IPA-HPLC; Flow rate: 20 mL/min; Gradient: isocratic 10; Wave Length: 206/210 nm; RT1 (min): 7; RT2 (min): 9; Sample Solvent: IPA-HPLC; Injection Volume: 0.3 mL; Number Of Runs: 6) to afford 7-((1R,3S)-3-(2-fluoro-6-methylphenyl)cyclopentyl)-3-methyl-5-((3-(trifluoromethyl)pyridin-2-yl)methyl)pyrido[2,3-b]pyrazin-6(5H)-one (6.1 mg, 6.01%) as a white solid. ¹H NMR (400 MHz, Chloroform-d) δ 8.44-8.38 (m, 1H), 8.31 (s, 1H), 7.99-7.92 (m, 1H), 7.88 (d, J=1.1 Hz, 1H), 7.25-7.18 (m, 1H), 7.07-6.98 (m, 1H), 6.93-6.81 (m, 2H), 6.03 (s, 2H), 3.81 (p, J=8.5 Hz, 1H), 3.58 (p, J=9.2 Hz, 1H), 2.50 (s, 3H), 2.46 (s, 1H), 2.37 (s, 3H), 2.34-2.29 (m, 1H), 2.17-2.04 (m, 3H), 1.96-1.88 (m, 1H). MS m/z: 497.10 [M+H]⁺.

171C: 7-((1S,3S)-3-(2-fluoro-6-methylphenyl)cyclopentyl)-3-methyl-5-((3-(trifluoromethyl)pyridin-2-yl)methyl)pyrido[2,3-b]pyrazin-6(5H)-one. The crude product (100 mg) was purified by Chiral-HPLC with the following conditions (Column: CHIRAL ART Cellulose-SZ, 2.0*25 cm, Sum; Mobile Phase A: Hex (10 mM NH₃-MeOH), Mobile Phase B: ACN:EtOH=2:1; Flow rate: 20 mL/min; Gradient: isocratic 10; Wave Length: 218/346 nm; RT1 (min): 5.3; RT2 (min): 8.2; Sample Solvent: EtOH; Injection Volume: 0.5 mL; Number Of Runs: 7) to afford 7-((1S,3S)-3-(2-fluoro-6-methylphenyl)cyclopentyl)-3-methyl-5-((3-(trifluoromethyl)pyridin-2-yl)methyl)pyrido[2,3-b]pyrazin-6(5H)-one (18.3 mg, 18.1%) as a white solid. ¹H NMR (400 MHz, Chloroform-d) δ 8.43 (d, J=4.7 Hz, 1H), 8.32 (s, 1H), 8.01-7.95 (m, 2H), 7.25-7.21 (m, 1H), 7.09-6.99 (m, 1H), 6.94-6.83 (m, 2H), 6.03 (s, 2H), 3.52 (p, J=8.6 Hz, 2H), 2.51 (s, 3H), 2.37 (s, 4H), 2.32-2.23 (m, 1H), 2.17-2.11 (m, 2H), 2.07-2.02 (m, 2H). MS m/z: 497.10 [M+H]⁺.

171D: 7-((1R,3R)-3-(2-fluoro-6-methylphenyl)cyclopentyl)-3-methyl-5-((3-(trifluoromethyl)pyridin-2-yl)methyl)pyrido[2,3-b]pyrazin-6(5H)-one. The crude product (100 mg) was purified by Chiral-HPLC with the following conditions (Column: CHIRAL ART Cellulose-SZ, 2.0*25 cm, Sum; Mobile Phase A: Hex (10 mM NH₃-MeOH), Mobile Phase B: ACN:EtOH=2:1; Flow rate: 20 mL/min; Gradient: isocratic 10; Wave Length: 218/346 nm; RT1 (min): 5.3; RT2 (min): 8.2; Sample Solvent: EtOH; Injection Volume: 0.5 mL; Number Of Runs: 7) to afford 7-((1R,3R)-3-(2-fluoro-6-methylphenyl)cyclopentyl)-3-methyl-5-((3-(trifluoromethyl)pyridin-2-yl)methyl)pyrido[2,3-b]pyrazin-6(5H)-one (23.2 mg, 23.0%) as a white solid. ¹H NMR (400 MHz, Chloroform-d) δ 8.45-8.39 (m, 1H), 8.31 (s, 1H), 8.00-7.93 (m, 2H), 7.25-7.19 (m, 1H), 7.09-6.99 (m, 1H), 6.95-6.83 (m, 2H), 6.03 (s, 2H), 3.59-3.45 (m, 2H), 2.50 (s, 3H), 2.37 (s, 4H), 2.33-2.22 (m, 1H), 2.18-2.01 (m, 4H). MS m/z: 497.10 [M+H]⁺.

### 7-((1S,3R)-3-(2-Fluoro-6-methylphenyl)cyclopentyl)-3-methyl-5-((3-methylpyrazin-2-yl)methyl)pyrido[2,3-b]pyrazin-6(5H)-one (172A); 7-((1R,3S)-3-(2-Fluoro-6-methylphenyl)cyclopentyl)-3-methyl-5-((3-methylpyrazin-2-yl)methyl)pyrido[2,3-b]pyrazin-6(5H)-one (172B); 7-((1S,3S)-3-(2-Fluoro-6-methylphenyl)cyclopentyl)-3-methyl-5-((3-methylpyrazin-2-yl)methyl)pyrido[2,3-b]pyrazin-6(5H)-one (172C); 7-((1R,3R)-3-(2-Fluoro-6-methylphenyl)cyclopentyl)-3-methyl-5-((3-methylpyrazin-2-yl)methyl)pyrido[2,3-b]pyrazin-6(5H)-one 172D)

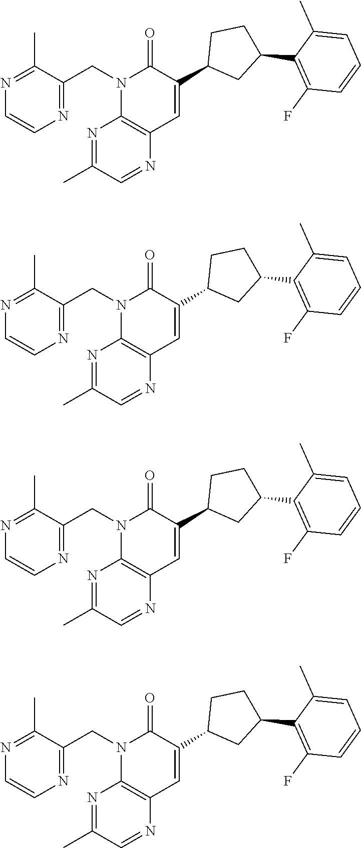 [SMILES: Cc1cnc2cc([C@H]3CC[C@@H](c4c(C)cccc4F)C3)c(=O)n(Cc3nccnc3C)c2n1 ; Cc1cnc2cc([C@@H]3CC[C@H](c4c(C)cccc4F)C3)c(=O)n(Cc3nccnc3C)c2n1 ; Cc1cnc2cc([C@H]3CC[C@H](c4c(C)cccc4F)C3)c(=O)n(Cc3nccnc3C)c2n1 ; Cc1cnc2cc([C@@H]3CC[C@@H](c4c(C)cccc4F)C3)c(=O)n(Cc3nccnc3C)c2n1]

Step 1: 7-(3-(2-fluoro-6-methylphenyl)cyclopentyl)-3-methyl-5-((3-methylpyrazin-2-yl)methyl)pyrido[2,3-b]pyrazin-6(5H)-one: Followed the General Procedure E using 7-[3-(2-fluoro-6-methylphenyl)cyclopentyl]-3-methyl-5H-pyrido[2,3-b]pyrazin-6-one (100 mg, 0.296 mmol, 1 equiv) and 2-(chloromethyl)-3-methylpyrazine hydrochloride (63.6 mg, 0.355 mmol, 1.2 equiv) as the starting materials to give the crude product 7-(3-(2-fluoro-6-methylphenyl)cyclopentyl)-3-methyl-5-((3-methylpyrazin-2-yl)methyl)pyrido[2,3-b]pyrazin-6(5H)-one (100 mg) as a white solid. MS m/z: 444 [M+H]⁺.

172A: 7-((1S,3R)-3-(2-fluoro-6-methylphenyl)cyclopentyl)-3-methyl-5-((3-methylpyrazin-2-yl)methyl)pyrido[2,3-b]pyrazin-6(5H)-one. The crude product (100 mg) was purified by Chiral-HPLC with the following conditions (Column: CHIRAL ART Cellulose-SC, 3*25 cm, 5 m; Mobile Phase A: Hex (10 mM NH₃-MeOH), Mobile Phase B: ACN:EtOH=2:1; Flow rate: 40 mL/min; Gradient: isocratic 50; Wave Length: 201/343 nm; RT1 (min): 8.04; RT2 (min): 9.325; Sample Solvent: MeOH:DCM=1:1-HPLC; Injection Volume: 0.4 mL; Number Of Runs: 6) to afford 7-((1S,3R)-3-(2-fluoro-6-methylphenyl)cyclopentyl)-3-methyl-5-((3-methylpyrazin-2-yl)methyl)pyrido[2,3-b]pyrazin-6(5H)-one (8.4 mg, 6.3%) as a white solid. ¹H NMR (400 MHz, Chloroform-d) δ 8.33 (s, 1H), 8.27 (d, J=2.6 Hz, 1H), 8.13 (d, J=2.6 Hz, 1H), 7.87 (d, J=1.2 Hz, 1H), 7.08-6.98 (m, 1H), 6.93-6.82 (m, 2H), 5.83 (s, 2H), 3.80 (p, J=8.7 Hz, 1H), 3.57 (p, J=9.3 Hz, 1H), 2.80 (s, 3H), 2.52 (s, 3H), 2.50-2.41 (m, 1H), 2.37 (s, 3H), 2.36-2.26 (m, 1H), 2.14-1.88 (m, 4H). MS m/z: 444.10 [M+H]⁺.

172B: 7-((1R,3S)-3-(2-fluoro-6-methylphenyl)cyclopentyl)-3-methyl-5-((3-methylpyrazin-2-yl)methyl)pyrido[2,3-b]pyrazin-6(5H)-one. The crude product (100 mg) was purified by Chiral-HPLC with the following conditions (Column: CHIRAL ART Cellulose-SC, 3*25 cm, 5 m; Mobile Phase A: Hex (10 mM NH₃-MeOH), Mobile Phase B: ACN:EtOH=2:1; Flow rate: 40 mL/min; Gradient: isocratic 50; Wave Length: 201/343 nm; RT1 (min): 8.04; RT2 (min): 9.325; Sample Solvent: MeOH:DCM=1:1-HPLC; Injection Volume: 0.4 mL; Number Of Runs: 6) to afford 7-((1R,3S)-3-(2-fluoro-6-methylphenyl)cyclopentyl)-3-methyl-5-((3-methylpyrazin-2-yl)methyl)pyrido[2,3-b]pyrazin-6(5H)-one (9.1 mg, 8.7%) as a white solid. ¹H NMR (400 MHz, Chloroform-d) δ 8.33 (s, 1H), 8.27 (d, J=2.6 Hz, 1H), 8.12 (d, J=2.6 Hz, 1H), 7.87 (d, J=1.1 Hz, 1H), 7.08-6.98 (m, 1H), 6.93-6.82 (m, 2H), 5.83 (s, 2H), 3.80 (p, J=8.5 Hz, 1H), 3.57 (p, J=9.2 Hz, 1H), 2.79 (s, 3H), 2.53 (s, 3H), 2.51-2.41 (m, 1H), 2.37 (s, 3H), 2.35-2.27 (m, 1H), 2.14-1.99 (m, 3H), 1.99-1.84 (m, 1H). MS m/z: 444.10 [M+H]⁺.

172C: 7-((1S,3S)-3-(2-fluoro-6-methylphenyl)cyclopentyl)-3-methyl-5-((3-methylpyrazin-2-yl)methyl)pyrido[2,3-b]pyrazin-6(5H)-one. The crude product (100 mg) was purified by Chiral-HPLC with the following conditions (Column: CHIRAL ART Cellulose-SC, 3*25 cm, 5 m; Mobile Phase A: Hex (10 mM NH₃-MeOH), Mobile Phase B: ACN:EtOH=2:1; Flow rate: 40 mL/min; Gradient: isocratic 50; Wave Length: 201/343 nm; RT1 (min): 8.04; RT2 (min): 9.325; Sample Solvent: MeOH:DCM=1:1-HPLC; Injection Volume: 0.4 mL; Number Of Runs: 6) to afford 7-((1S,3S)-3-(2-fluoro-6-methylphenyl)cyclopentyl)-3-methyl-5-((3-methylpyrazin-2-yl)methyl)pyrido[2,3-b]pyrazin-6(5H)-one (24.9 mg, 24.7%) as a white solid. ¹H NMR (400 MHz, Chloroform-d) δ 8.33 (s, 1H), 8.27 (d, J=2.5 Hz, 1H), 8.14 (d, J=2.5 Hz, 1H), 7.95 (d, J=1.1 Hz, 1H), 7.09-6.99 (m, 1H), 6.95-6.83 (m, 2H), 5.83 (s, 2H), 3.59-3.47 (m, 2H), 2.81 (s, 3H), 2.52 (s, 3H), 2.37 (s, 4H), 2.28-2.23 (m, 1H), 2.19-2.10 (m, 2H), 2.07-1.96 (m, 2H). MS m/z: 444.10 [M+H]⁺.

172D: 7-((1R,3R)-3-(2-fluoro-6-methylphenyl)cyclopentyl)-3-methyl-5-((3-methylpyrazin-2-yl)methyl)pyrido[2,3-b]pyrazin-6(5H)-one. The crude product (100 mg) was purified by Chiral-HPLC with the following conditions (Column: CHIRAL ART Cellulose-SC, 3*25 cm, 5 m; Mobile Phase A: Hex (10 mM NH₃-MeOH), Mobile Phase B: ACN:EtOH=2:1; Flow rate: 40 mL/min; Gradient: isocratic 50; Wave Length: 201/343 nm; RT1 (min): 8.04; RT2 (min): 9.325; Sample Solvent: MeOH:DCM=1:1-HPLC; Injection Volume: 0.4 mL; Number Of Runs: 6) to afford 7-((1R,3R)-3-(2-fluoro-6-methylphenyl)cyclopentyl)-3-methyl-5-((3-methylpyrazin-2-yl)methyl)pyrido[2,3-b]pyrazin-6(5H)-one (22.5 mg, 22.3%) as a white solid. ¹H NMR (400 MHz, Chloroform-d) δ 8.33 (s, 1H), 8.27 (d, J=2.6 Hz, 1H), 8.12 (d, J=2.6 Hz, 1H), 7.95 (d, J=1.0 Hz, 1H), 7.09-6.99 (m, 1H), 6.95-6.83 (m, 2H), 5.83 (s, 2H), 3.58-3.45 (m, 2H), 2.80 (s, 3H), 2.52 (s, 3H), 2.37 (s, 4H), 2.31-2.23 (m, 1H), 2.19-2.09 (m, 2H), 2.09-1.95 (m, 2H). MS m/z: 444.10 [M+H]⁺.

### 7-((1r,4r)-4-(2-Fluoro-6-methylphenyl)cyclohexyl)-5-((3-methoxypyrazin-2-yl)methyl)-3-methylpyrido[2,3-b]pyrazin-6(5H)-one (173)

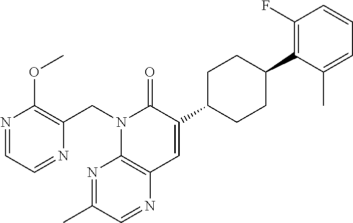 [SMILES: COc1nccnc1Cn1c(=O)c([C@H]2CC[C@H](c3c(C)cccc3F)CC2)cc2ncc(C)nc21]

Step 1: 7-((1r,4r)-4-(2-fluoro-6-methylphenyl)cyclohexyl)-5-((3-methoxypyrazin-2-yl)methyl)-3-methylpyrido[2,3-b]pyrazin-6(5H)-one: Followed the General Procedure E using 7-((1r,4r)-4-(2-fluoro-6-methylphenyl)cyclohexyl)-3-methylpyrido[2,3-b]pyrazin-6(5H)-one (35 mg, 0.100 mmol, 1 equiv.) and 2-(bromomethyl)-3-methoxypyrazine hydrogen chloride (28.5 mg, 0.119 mmol, 1.2 equiv.) as the starting materials to give 7-((1r,4r)-4-(2-fluoro-6-methylphenyl)cyclohexyl)-5-((3-methoxypyrazin-2-yl)methyl)-3-methylpyrido[2,3-b]pyrazin-6(5H)-one (19.6 mg, 38.4%) as a white solid. ¹H NMR (400 MHz, Chloroform-d) δ 8.32 (s, 1H), 7.93 (d, J=2.8 Hz, 1H), 7.87-7.77 (m, 2H), 7.07-6.99 (m, 1H), 6.95-6.90 (m, 1H), 6.88-6.79 (m, 1H), 5.84 (s, 2H), 4.09 (s, 3H), 3.13 (tt, J=12.0, 2.9 Hz, 1H), 2.90 (t, J=12.0 Hz, 1H), 2.54 (s, 3H), 2.38 (s, 3H), 2.20-2.10 (m, 4H), 1.89-1.79 (m, 2H), 1.61-1.46 (m, 2H). MS m/z: 474.15 [M+H]⁺.

### 5-((S)-8,8-Difluoro-5,6,7,8-tetrahydroquinoxalin-5-yl)-7-((1r,4S)-4-(2-fluoro-6-methylphenyl)cyclohexyl)-3-methylpyrido[2,3-b]pyrazin-6(5H)-one (174A); 5-((R)-8,8-Difluoro-5,6,7,8-tetrahydroquinoxalin-5-yl)-7-((1r,4R)-4-(2-fluoro-6-methylphenyl)cyclohexyl)-3-methylpyrido[2,3-b]pyrazin-6(5H)-one (174B)

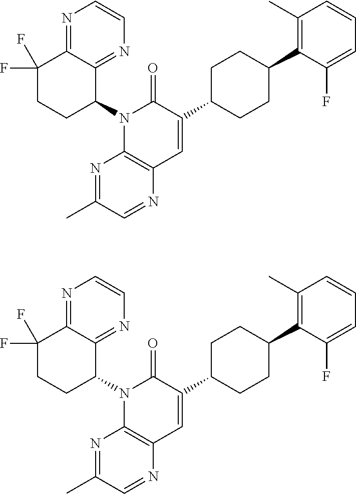 [SMILES: Cc1cnc2cc([C@H]3CC[C@H](c4c(C)cccc4F)CC3)c(=O)n([C@H]3CCC(F)(F)c4nccnc43)c2n1 ; Cc1cnc2cc([C@H]3CC[C@H](c4c(C)cccc4F)CC3)c(=O)n([C@@H]3CCC(F)(F)c4nccnc43)c2n1]

Step 1: 5-(8,8-difluoro-5,6,7,8-tetrahydroquinoxalin-5-yl)-7-((1r,4r)-4-(2-fluoro-6-methylphenyl)cyclohexyl)-3-methylpyrido[2,3-b]pyrazin-6(5H)-one: Followed the General Procedure E using 7-((1r,4r)-4-(2-fluoro-6-methylphenyl)cyclohexyl)-3-methylpyrido[2,3-b]pyrazin-6(5H)-one (100 mg, 0.285 mmol, 1 equiv) and 8-bromo-5,5-difluoro-5,6,7,8-tetrahydroquinoxaline (85 mg, 0.342 mmol, 1.2 equiv) as the starting materials to give the crude product 5-(8,8-difluoro-5,6,7,8-tetrahydroquinoxalin-5-yl)-7-((1r,4r)-4-(2-fluoro-6-methylphenyl)cyclohexyl)-3-methylpyrido[2,3-b]pyrazin-6(5H)-one (50 mg, 33%) as a white solid. The crude product (50 mg) was purified by Chiral-HPLC with the following conditions (Column: CHIRAL ART Cellulose-SZ, 3*25 cm, 5 m; Mobile Phase A: Hex (10 mM NH₃-MeOH), Mobile Phase B: IPA-HPLC; Flow rate: 40 mL/min; Gradient: isocratic 10; Wave Length: 215/68 nm; RT1 (min): 12.5; RT2 (min): 14.93; Sample Solvent: EtOH-HPLC; Sample concentration: mg/mL; Injection Volume: 0.3 mL; Number Of Runs: 6) to afford 5-((S)-8,8-difluoro-5,6,7,8-tetrahydroquinoxalin-5-yl)-7-((1r,4S)-4-(2-fluoro-6-methylphenyl)cyclohexyl)-3-methylpyrido[2,3-b]pyrazin-6(5H)-one (12.6 mg, 24.9%) as a white solid and 5-((R)-8,8-difluoro-5,6,7,8-tetrahydroquinoxalin-5-yl)-7-((1r,4R)-4-(2-fluoro-6-methylphenyl)cyclohexyl)-3-methylpyrido[2,3-b]pyrazin-6(5H)-one (11.2 mg, 22.1%) as a white solid.

174A: 5-((S)-8,8-difluoro-5,6,7,8-tetrahydroquinoxalin-5-yl)-7-((1r,4S)-4-(2-fluoro-6-methylphenyl)cyclohexyl)-3-methylpyrido[2,3-b]pyrazin-6(5H)-one. ¹H NMR (400 MHz, Chloroform-d) δ 8.63-8.50 (m, 1H), 8.48-8.38 (m, 1H), 8.36-8.13 (m, 1H), 7.82 (d, J=5.0 Hz, 1H), 7.13-6.99 (m, 1H), 6.96-6.77 (m, 2H), 3.26-2.65 (m, 5H), 2.62-2.47 (m, 1H), 2.38 (d, J=16.6 Hz, 3H), 2.30-2.00 (m, 5H), 1.98-1.69 (m, 5H), 1.67-1.37 (m, 2H). MS m/z: 520.15 [M+H]⁺.

174B: 5-((R)-8,8-difluoro-5,6,7,8-tetrahydroquinoxalin-5-yl)-7-((1r,4R)-4-(2-fluoro-6-methylphenyl)cyclohexyl)-3-methylpyrido[2,3-b]pyrazin-6(5H)-one. ¹H NMR (400 MHz, Chloroform-d) δ 8.63-8.51 (m, 1H), 8.44 (d, J=18.4 Hz, 1H), 8.25 (d, J=66.5 Hz, 1H), 7.81 (d, J=5.6 Hz, 1H), 7.12-7.01 (m, 1H), 6.94-6.79 (m, 2H), 3.24-2.65 (m, 6H), 2.60-2.47 (m, 1H), 2.38 (d, J=16.6 Hz, 3H), 2.30-2.03 (m, 6H), 1.94 (d, J=22.9 Hz, 3H), 1.66-1.54 (m, 1H), 1.48-1.40 (m, 1H). MS m/z: 520.25 [M+H]⁺.

### 5-((3-(Difluoromethoxy)pyridin-2-yl)methyl)-7-((1S,3R)-3-(2-fluoro-6-methylphenyl)cyclopentyl)-3-methylpyrido[2,3-b]pyrazin-6(5H)-one (175A); 5-((3-(Difluoromethoxy)pyridin-2-yl)methyl)-7-((1R,3S)-3-(2-fluoro-6-methylphenyl)cyclopentyl)-3-methylpyrido[2,3-b]pyrazin-6(5H)-one (175B); 5-((3-(Difluoromethoxy)pyridin-2-yl)methyl)-7-((1S,3S)-3-(2-fluoro-6-methylphenyl)cyclopentyl)-3-methylpyrido[2,3-b]pyrazin-6(5H)-one (175C); 5-((3-(Difluoromethoxy)pyridin-2-yl)methyl)-7-((1R,3R)-3-(2-fluoro-6-methylphenyl)cyclopentyl)-3-methylpyrido[2,3-b]pyrazin-6(5H)-one (175D)

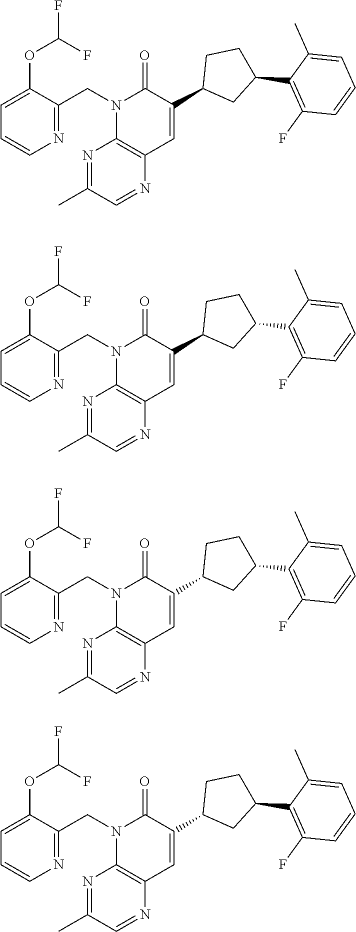

Step 1: 5-((3-(difluoromethoxy)pyridin-2-yl)methyl)-7-(3-(2-fluoro-6-methylphenyl)cyclopentyl)-3-methylpyrido[2,3-b]pyrazin-6(5H)-one: Followed the General Procedure E using 7-(3-(2-fluoro-6-methylphenyl)cyclopentyl)-3-methylpyrido[2,3-b]pyrazin-6(5H)-one (150 mg, 0.445 mmol, 1 equiv) and 2-(bromomethyl)-3-(difluoromethoxy)pyridine (127 mg, 0.534 mmol, 1.2 equiv) as the starting materials to afford the crude product 5-((3-(difluoromethoxy)pyridin-2-yl)methyl)-7-(3-(2-fluoro-6-methylphenyl)cyclopentyl)-3-methylpyrido[2,3-b]pyrazin-6(5H)-one (100 mg) as a white solid. The crude product was purified by Chiral-HPLC with the following conditions (Column: CHIRAL ART Cellulose-SC, 3*25 cm, 5 m; Mobile Phase A: Hex (10 mM NH₃-MeOH), Mobile Phase B: EtOH-HPLC; Flow rate: 40 mL/min; Gradient: isocratic 25; Wave Length: 204/264 nm; RT1 (min): 6.665; RT2 (min): 7.665; RT3 (min): 8.13; RT4 (min): 11.1; Sample Solvent: EtOH-HPLC; Injection Volume: 0.2 mL; Number Of Runs: 10) to afford 5-((3-(difluoromethoxy)pyridin-2-yl)methyl)-7-((1S,3R)-3-(2-fluoro-6-methylphenyl)cyclopentyl)-3-methylpyrido[2,3-b]pyrazin-6(5H)-one (5.9 mg, 5.7%), 5-((3-(difluoromethoxy)pyridin-2-yl)methyl)-7-((1R,3S)-3-(2-fluoro-6-methylphenyl)cyclopentyl)-3-methylpyrido[2,3-b]pyrazin-6(5H)-one (6.3 mg, 6.2%), 5-((3-(difluoromethoxy)pyridin-2-yl)methyl)-7-((1S,3S)-3-(2-fluoro-6-methylphenyl)cyclopentyl)-3-methylpyrido[2,3-b]pyrazin-6(5H)-one (18.8 mg, 18.5%) and 5-((3-(difluoromethoxy)pyridin-2-yl)methyl)-7-((1R,3R)-3-(2-fluoro-6-methylphenyl)cyclopentyl)-3-methylpyrido[2,3-b]pyrazin-6(5H)-one (18.1 mg, 17.6%) as a white solid.

175A: 5-((3-(difluoromethoxy)pyridin-2-yl)methyl)-7-((1S,3R)-3-(2-fluoro-6-methylphenyl)cyclopentyl)-3-methylpyrido[2,3-b]pyrazin-6(5H)-one: ¹H NMR (400 MHz, Chloroform-d) δ 8.32 (s, 1H), 8.23-8.17 (m, 1H), 7.86 (d, J=1.0 Hz, 1H), 7.57-7.46 (m, 1H), 7.20-7.12 (m, 1H), 7.07-6.98 (m, 1H), 6.91-6.54 (m, 3H), 5.92 (s, 2H), 3.85-3.72 (m, 1H), 3.63-3.49 (m, 1H), 2.54 (s, 3H), 2.49-2.41 (m, 1H), 2.36 (s, 3H), 2.35-2.26 (m, 1H), 2.15-2.02 (m, 3H), 1.94-1.85 (m, 1H). MS m/z: 495.10 [M+H]⁺.

175B: 5-((3-(difluoromethoxy)pyridin-2-yl)methyl)-7-((1R,3S)-3-(2-fluoro-6-methylphenyl)cyclopentyl)-3-methylpyrido[2,3-b]pyrazin-6(5H)-one. ¹H NMR (400 MHz, Chloroform-d) δ 8.33 (s, 1H), 8.27 (d, J=4.8 Hz, 1H), 7.88 (s, 1H), 7.61 (d, J=8.2 Hz, 1H), 7.28 (s, 1H), 7.10-6.98 (m, 1H), 6.92-6.54 (m, 3H), 5.96 (s, 2H), 3.84-3.71 (m, 1H), 3.63-3.49 (m, 1H), 2.55 (s, 3H), 2.48-2.41 (m, 1H), 2.36 (s, 3H), 2.33-2.28 (m, 1H), 2.14-2.01 (m, 3H), 1.94-1.84 (m, 1H). MS m/z: 495.10 [M+H]⁺.

175C: 5-((3-(difluoromethoxy)pyridin-2-yl)methyl)-7-((1S,3S)-3-(2-fluoro-6-methylphenyl)cyclopentyl)-3-methylpyrido[2,3-b]pyrazin-6(5H)-one. ¹H NMR (400 MHz, Chloroform-d) δ 8.33 (s, 1H), 8.26-8.20 (m, 1H), 7.94 (d, J=1.0 Hz, 1H), 7.58-7.50 (m, 1H), 7.24-7.16 (m, 1H), 7.09-6.99 (m, 1H), 6.93-6.54 (m, 3H), 5.94 (s, 2H), 3.58-3.43 (m, 2H), 2.54 (s, 3H), 2.36 (s, 4H), 2.30-2.22 (m, 1H), 2.16-2.10 (m, 2H), 2.05-1.98 (m, 2H). MS m/z: 495.10 [M+H]⁺.

175D: 5-((3-(difluoromethoxy)362yridine-2-yl)methyl)-7-((1R,3R)-3-(2-fluoro-6-methylphenyl)cyclopentyl)-3-methylpyrido[2,3-b]pyrazin-6(5H)-one. ¹H NMR (400 MHz, Chloroform-d) δ 8.33 (s, 1H), 8.25-8.19 (m, 1H), 7.94 (d, J=1.0 Hz, 1H), 7.56-7.49 (m, 1H), 7.22-7.14 (m, 1H), 7.10-6.99 (m, 1H), 6.93-6.55 (m, 3H), 5.93 (s, 2H), 3.58-3.44 (m, 2H), 2.54 (s, 3H), 2.37 (s, 4H), 2.29-2.21 (m, 1H), 2.16-1.99 (m, 4H). MS m/z: 495.10 [M+H]⁺.

### 5-((3-(Difluoromethyl)pyridin-2-yl)methyl)-7-((1r,4r)-4-(2-fluoro-6-methylphenyl)cyclohexyl)-3-methylpyrido[2,3-b]pyrazin-6(5H)-one (176)

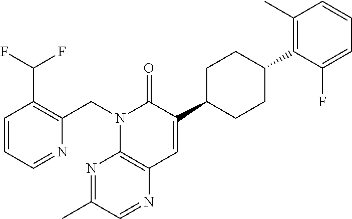 [SMILES: Cc1cnc2cc([C@H]3CC[C@H](c4c(C)cccc4F)CC3)c(=O)n(Cc3ncccc3C(F)F)c2n1]

Step 1: 5-((3-(difluoromethyl)pyridin-2-yl)methyl)-7-((1r,4r)-4-(2-fluoro-6-methylphenyl)cyclohexyl)-3-methylpyrido[2,3-b]pyrazin-6(5H)-one: Followed the General Procedure E using 7-((1r,4r)-4-(2-fluoro-6-methylphenyl)cyclohexyl)-3-methylpyrido[2,3-b]pyrazin-6(5H)-one (45 mg, 0.128 mmol, 1 equiv) and 2-(bromomethyl)-3-(difluoromethyl)pyridine (42.6 mg, 0.192 mmol, 1.5 equiv) as the starting materials to give 5-((3-(difluoromethyl)pyridin-2-yl)methyl)-7-((1r,4r)-4-(2-fluoro-6-methylphenyl)cyclohexyl)-3-methylpyrido[2,3-b]pyrazin-6(5H)-one (34.3 mg, 53.6%) as a white solid. ¹H NMR (400 MHz, Chloroform-d) δ 8.46 (d, J=4.9 Hz, 1H), 8.34 (s, 1H), 7.94 (d, J=7.8 Hz, 1H), 7.80 (s, 1H), 7.43-7.27 (m, 2H), 7.03 (td, J=7.9, 5.5 Hz, 1H), 6.92 (d, J=7.4 Hz, 1H), 6.84 (dd, J=11.8, 8.1 Hz, 1H), 5.89 (s, 2H), 3.12 (dd, J=13.4, 10.6 Hz, 1H), 3.00-2.81 (m, 1H), 2.57 (s, 3H), 2.37 (s, 3H), 2.26-2.08 (m, 4H), 1.91-1.79 (m, 2H), 1.60-1.45 (m, 2H). MS m/z: 493.15 [M+H]⁺.

### 5-((3-(1,1-Difluoroethyl)pyridin-2-yl)methyl)-7-((1r,4r)-4-(2-fluoro-6-methylphenyl)cyclohexyl)-3-methylpyrido[2,3-b]pyrazin-6(5H)-one (177)

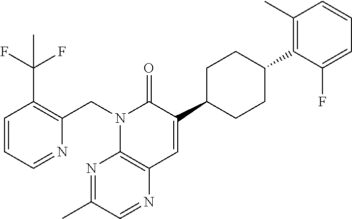 [SMILES: Cc1cnc2cc([C@H]3CC[C@H](c4c(C)cccc4F)CC3)c(=O)n(Cc3ncccc3C(C)(F)F)c2n1]

Step 1: 2-chloro-3-(1,1-difluoroethyl)pyridine: Followed the General Procedure AE using 1-(2-chloropyridin-3-yl)ethan-1-one (2 g, 12.9 mmol, 1.0 equiv) as the starting materials to give 2-chloro-3-(1,1-difluoroethyl)pyridine (1.1 g, 48%) as a yellow oil. MS m/z: 178 [M+H]⁺.

Step 2: (3-(1,1-difluoroethyl)pyridin-2-yl)methanol: Followed the General Procedure V using 2-chloro-3-(1,1-difluoroethyl)pyridine (1.1 g, 6.21 mmol, 1.1 equiv) and (tributylstannyl)methanol (2.2 g, 6.83 mmol, 1.1 equiv) as the starting materials to give (3-(1,1-difluoroethyl)pyridin-2-yl)methanol (180 mg, 16%) as a yellow oil. MS m/z: 174 [M+H]⁺.

Step 3: 2-(chloromethyl)-3-(1,1-difluoroethyl)pyridine: Followed the General Procedure J using (3-(1,1-difluoroethyl)pyridin-2-yl)methanol (180 mg) as the starting material to give the crude product 2-(chloromethyl)-3-(1,1-difluoroethyl)pyridine (160 mg). MS m/z: 192 [M+H]⁺.

Step 4: 5-((3-ethoxypyrazin-2-yl)methyl)-7-((1r,4r)-4-(2-fluoro-6-methylphenyl)cyclohexyl)-3-methylpyrido[2,3-b]pyrazin-6(5H)-one: Followed the General Procedure E using 2-(chloromethyl)-3-(1,1-difluoroethyl)pyridine (21.2 mg, 111 μmol, 1.1 equiv) and 7-((1r,4r)-4-(2-fluoro-6-methylphenyl)cyclohexyl)-3-methylpyrido[2,3-b]pyrazin-6(5H)-one (30 mg, 85 μmol, 1.0 equiv) as the starting materials to give 5-((3-ethoxypyrazin-2-yl)methyl)-7-((1r,4r)-4-(2-fluoro-6-methylphenyl)cyclohexyl)-3-methylpyrido[2,3-b]pyrazin-6(5H)-one (10.5 mg, 7.3%) as a white solid. ¹H NMR (400 MHz, Chloroform-d) δ 8.48-8.42 (m, 1H), 8.35 (s, 1H), 7.98 (d, J=7.6 Hz, 1H), 7.86 (s, 1H), 7.33 (dd, J=7.9, 5.0 Hz, 1H), 7.03 (td, J=7.9, 5.5 Hz, 1H), 6.92 (d, J=7.4 Hz, 1H), 6.88-6.79 (m, 1H), 6.01 (s, 2H), 3.11 (t, J=12.1 Hz, 1H), 2.90 (t, J=12.3 Hz, 1H), 2.55 (s, 3H), 2.37 (s, 3H), 2.26 (t, J=18.4 Hz, 3H), 2.15 (d, J=12.3 Hz, 4H), 1.84 (d, J=12.3 Hz, 2H), 1.54 (q, J=12.5 Hz, 2H). MS m/z: 507.15 [M+H]⁺.

### 7-((1r,4r)-4-(4-(Difluoromethyl)pyridin-3-yl)cyclohexyl)-3-methyl-5-((3-(trifluoromethyl)pyridin-2-yl)methyl)pyrido[2,3-b]pyrazin-6(5H)-one (178)

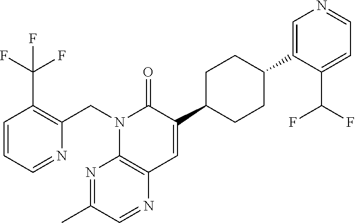 [SMILES: Cc1cnc2cc([C@H]3CC[C@H](c4cnccc4C(F)F)CC3)c(=O)n(Cc3ncccc3C(F)(F)F)c2n1]

Step 1: 3-bromo-4-(difluoromethyl)pyridine: Followed the General Procedure AE using 3-bromoisonicotinaldehyde (1 g, 5.4 mmol, 1.0 equiv) as the starting material to give 3-bromo-4-(difluoromethyl)pyridine (700 mg, 62%) as a white solid. MS m/z: 208 [M+H]⁺.

Step 2: ethyl 2-(4-(4-(difluoromethyl)pyridin-3-yl)cyclohex-3-en-1-yl)acetate: Followed the General Procedure AL using 3-bromo-4-(difluoromethyl)pyridine (700 mg, 3.38 mmol, 1.0 equiv) and ethyl 2-(4-(4,4,5,5-tetramethyl-1,3,2-dioxaborolan-2-yl)cyclohex-3-en-1-yl)acetate (994 mg, 3.38 mmol, 1.0 equiv.) as the starting materials to give ethyl 2-(4-(4-(difluoromethyl)pyridin-3-yl)cyclohex-3-en-1-yl)acetate (720 mg, 72%). MS m/z: 296 [M+H]⁺.

Step 3: ethyl 2-(4-(4-(difluoromethyl)pyridin-3-yl)cyclohexyl)acetate: Followed the General Procedure AR using ethyl 2-(4-(4-(difluoromethyl)pyridin-3-yl)cyclohex-3-en-1-yl)acetate (720 mg, 2.44 mmol, 1.0 equiv.) as the starting material to give the crude product ethyl 2-(4-(4-(difluoromethyl)pyridin-3-yl)cyclohexyl)acetate (680 mg). MS m/z: 298 [M+H]⁺.

Step 4: 7-((1r,4r)-4-(4-(difluoromethyl)pyridin-3-yl)cyclohexyl)-3-methylpyrido[2,3-b]pyrazin-6(5H)-one: Followed the General Procedure D using ethyl 2-(4-(4-(difluoromethyl)pyridin-3-yl)cyclohexyl)acetate (680 mg, 2.28 mmol, 1.0 equiv.) and 3-amino-5-methylpyrazine-2-carbaldehyde (375 mg, 2.73 mmol, 1.2 equiv.) as the starting material to give the crude product 7-(4-(4-(difluoromethyl)pyridin-3-yl)cyclohexyl)-3-methylpyrido[2,3-b]pyrazin-6(5H)-one (300 mg). The crude product was purified by Prep-HPLC with the following conditions (Column: XBridge Shield RP18 OBD Column 30*150 mm, 5 m; Mobile Phase A: Water (0.1% FA), Mobile Phase B: ACN; Flow rate: 60 mL/min mL/min; Gradient: 49% B to 60% B in 8 min; Wave Length: 254 nm/220 nm nm; RT1 (min): 7.00/8.93) to afford 7-((1r,4r)-4-(4-(difluoromethyl)pyridin-3-yl)cyclohexyl)-3-methylpyrido[2,3-b]pyrazin-6(51)-one (180 mg, 13%) as a white solid. MS m/z: 371 [M+H]⁺.

Step 5: 3-methyl-7-((1r,4r)-4-(4-methylthiazol-5-yl)cyclohexyl)-5-((3-(trifluoromethyl)pyridin-2-yl)methyl)pyrido[2,3-b]pyrazin-6(5H)-one: Followed the General Procedure E using 7-((1r,4r)-4-(4-(difluoromethyl)pyridin-3-yl)cyclohexyl)-3-methylpyrido[2,3-b]pyrazin-6(5H)-one (110 mg, 296 μmol, 1.0 equiv) and 2-(chloromethyl)-3-(trifluoromethyl)pyridine (57 mg, 296 μmol, 1.0 equiv) as the starting materials to give 3-methyl-7-((1r,4r)-4-(4-methylthiazol-5-yl)cyclohexyl)-5-((3-(trifluoromethyl)pyridin-2-yl)methyl)pyrido[2,3-b]pyrazin-6(5H)-one (15.1 mg, 9.6%) as a white solid. ¹H NMR (400 MHz, Chloroform-d) δ 8.73 (s, 1H), 8.60 (s, 1H), 8.43 (d, J=4.8 Hz, 1H), 8.34 (s, 1H), 8.01-7.94 (m, 1H), 7.81 (s, 1H), 7.48 (s, 1H), 7.24-7.20 (m, 1H), 6.87 (t, J=54.6 Hz, 1H), 6.03 (s, 2H), 3.15 (t, J=12.0 Hz, 1H), 2.91 (d, J=12.1 Hz, 1H), 2.52 (s, 3H), 2.22 (d, J=12.7 Hz, 2H), 2.04 (d, J=12.7 Hz, 2H), 1.84 (d, J=12.4 Hz, 2H), 1.62 (d, J=12.1 Hz, 2H). MS m/z: 530.1 [M+H]⁺.

### 7-((1r,4r)-4-(3-(Difluoromethyl)pyridin-2-yl)cyclohexyl)-3-methyl-5-((3-(trifluoromethyl)pyridin-2-yl)methyl)pyrido[2,3-b]pyrazin-6(5H)-one (179)

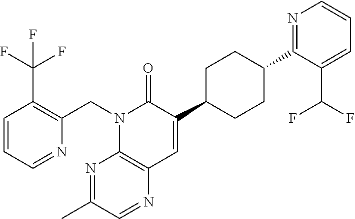 [SMILES: Cc1cnc2cc([C@H]3CC[C@H](c4ncccc4C(F)F)CC3)c(=O)n(Cc3ncccc3C(F)(F)F)c2n1]

Step 1: ethyl 2-(4-(3-(difluoromethyl)pyridin-2-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (500 mg, 1.70 mmol, 1.0 equiv.) and 2-bromo-3-(difluoromethyl)pyridine (389 mg, 1.87 mmol, 1.1 equiv.) as the starting materials to give ethyl 2-(4-(3-(difluoromethyl)pyridin-2-yl)cyclohex-3-en-1-yl)acetate (500 mg, 93%) as a light yellow oil. MS m/z: 296 [M+H]⁺.

Step 2: ethyl 2-(4-(3-(difluoromethyl)pyridin-2-yl)cyclohexyl)acetate: Followed the General Procedure AR using ethyl 2-(4-(3-(difluoromethyl)pyridin-2-yl)cyclohex-3-en-1-yl)acetate (500 mg, 1.69 mmol, 1.0 equiv.) as the starting material to give the crude product ethyl 2-(4-(3-(difluoromethyl)pyridin-2-yl)cyclohexyl)acetate (450 mg) as a light yellow oil. MS m/z: 298 [M+H]⁺.

Step 3: 7-((1r,4r)-4-(3-(difluoromethyl)pyridin-2-yl)cyclohexyl)-3-methylpyrido[2,3-b]pyrazin-6(5H)-one: Followed the General Procedure D using ethyl 2-(4-(3-(difluoromethyl)pyridin-2-yl)cyclohexyl)acetate (450 mg, 1.51 mmol, 1.0 equiv.) and 3-amino-5-methylpyrazine-2-carbaldehyde (249 mg, 1.82 mmol, 1.2 equiv.) as the starting materials to give 7-((1r,4r)-4-(3-(difluoromethyl)pyridin-2-yl)cyclohexyl)-3-methylpyrido[2,3-b]pyrazin-6(5H)-one (300 mg, 53%) as a light yellow solid. MS m/z: 371 [M+H]⁺.

Step 4: 7-((1r,4r)-4-(3-(difluoromethyl)pyridin-2-yl)cyclohexyl)-3-methyl-5-((3-(trifluoromethyl)pyridin-2-yl)methyl)pyrido[2,3-b]pyrazin-6(5H)-one: Followed the General Procedure E using 7-((1r,4r)-4-(3-(difluoromethyl)pyridin-2-yl)cyclohexyl)-3-methylpyrido[2,3-b]pyrazin-6(5H)-one (150 mg, 0.405 mmol, 1.0 equiv.) and 2-(chloromethyl)-3-(trifluoromethyl)pyridine (95 mg, 0.486 mmol, 1.2 equiv.) as the starting materials to give 7-((1r,4r)-4-(3-(difluoromethyl)pyridin-2-yl)cyclohexyl)-3-methyl-5-((3-(trifluoromethyl)pyridin-2-yl)methyl)pyrido[2,3-b]pyrazin-6(5H)-one (61.3 mg, 27.2%) as a white solid. ¹H NMR (300 MHz, Chloroform-d) δ 8.74-8.58 (m, 1H), 8.49-8.38 (m, 1H), 8.35 (s, 1H), 8.04 (d, J=1.1 Hz, 1H), 8.01-7.93 (m, 1H), 7.88 (d, J=8.0 Hz, 1H), 7.24 (dt, J=8.6, 4.4 Hz, 2H), 6.92 (t, J=55.2 Hz, 1H), 6.05 (s, 2H), 3.40-3.15 (m, 2H), 2.53 (s, 3H), 2.39-2.25 (m, 2H), 2.20-2.06 (m, 2H), 1.93 (tq, J=13.0, 4.1 Hz, 4H). MS m/z: 530.15 [M+H]⁺.

### 3-Methyl-7-((1r,4r)-4-(3-(trifluoromethyl)pyridin-2-yl)cyclohexyl)-5-((3-(trifluoromethyl)pyridin-2-yl)methyl)pyrido[2,3-b]pyrazin-6(5H)-one (180)

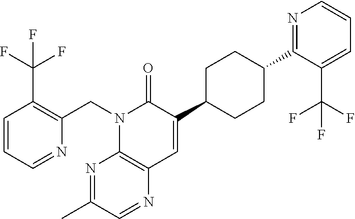 [SMILES: Cc1cnc2cc([C@H]3CC[C@H](c4ncccc4C(F)(F)F)CC3)c(=O)n(Cc3ncccc3C(F)(F)F)c2n1]

Step 1: ethyl 2-(4-(3-(trifluoromethyl)pyridin-2-yl)cyclohex-3-en-1-yl)acetate: Followed the General Procedure AL using 2-bromo-3-(trifluoromethyl) pyridine (500 mg, 2.21 mmol, 1 equiv.) and ethyl 2-(4-(4,4,5,5-tetramethyl-1,3,2-dioxaborolan-2-yl)cyclohex-3-en-1-yl)acetate (976 mg, 3.31 mmol, 1.50 equiv.) as the starting materials to give ethyl 2-(4-(3-(trifluoromethyl)pyridin-2-yl)cyclohex-3-en-1-yl)acetate (450 mg, 65%) as a brown oil. MS m/z: 314 [M+H]⁺.

Step 2: ethyl 2-(4-(3-(trifluoromethyl)pyridin-2-yl)cyclohexyl)acetate: Followed the General Procedure AR using ethyl 2-(4-(3-(trifluoromethyl)pyridin-2-yl)cyclohex-3-en-1-yl)acetate (500 mg, 1.69 mmol, 1.0 equiv.) as the starting material to give the crude product ethyl 2-(4-(3-(trifluoromethyl)pyridin-2-yl)cyclohexyl)acetate (450 mg) as a light yellow oil. MS m/z: 316 [M+H]⁺.

Step 3: 3-methyl-7-((1r,4r)-4-(3-(trifluoromethyl)pyridin-2-yl)cyclohexyl)pyrido[2,3-b]pyrazin-6(5H)-one: Followed the General Procedure D using ethyl 2-(4-(3-(trifluoromethyl)pyridin-2-yl)cyclohexyl)acetate (450 mg, 1.51 mmol, 1.0 equiv.) and 3-amino-5-methylpyrazine-2-carbaldehyde (249 mg, 1.82 mmol, 1.2 equiv.) as the starting materials to give 7-((1r,4r)-4-(3-(difluoromethyl)pyridin-2-yl)cyclohexyl)-3-methylpyrido[2,3-b]pyrazin-6(5H)-one (300 mg, 53%) as a light yellow solid. MS m/z: 389 [M+H]⁺.

Step 4: 3-methyl-7-((1r,4r)-4-(3-(trifluoromethyl) pyridin-2-yl) cyclohexyl)-5-((3-(trifluoromethyl) pyridin-2-yl) methyl) pyrido[2,3-b]pyrazin-6(5H)-one: Followed the General Procedure E using 3-methyl-7-((1r,4r)-4-(3-(trifluoromethyl)pyridin-2-yl)cyclohexyl)pyrido[2,3-b]pyrazin-6(5H)-one (60 mg, 0.154 mmol, 1.0 equiv.) and 2-(chloromethyl)-3-(trifluoromethyl)pyridine (36 mg, 0.185 mmol, 1.2 equiv.) as the starting materials to give 3-methyl-7-((1r,4r)-4-(3-(trifluoromethyl)pyridin-2-yl)cyclohexyl)-5-((3-(trifluoromethyl)pyridin-2-yl)methyl)pyrido[2,3-b]pyrazin-6(5H)-one (15.4 mg, 17.9%) as a white solid. ¹H NMR (300 MHz, Chloroform-d) δ 8.73 (d, J=4.8 Hz, 1H), 8.47-8.40 (m, 1H), 8.36 (s, 1H), 8.12 (d, J=1.2 Hz, 1H), 8.02-7.89 (m, 2H), 7.27-7.20 (m, 2H), 6.05 (s, 2H), 3.37 (td, J=11.6, 11.1, 5.7 Hz, 2H), 2.53 (s, 3H), 2.45-2.30 (m, 2H), 2.21-2.04 (m, 2H), 2.05-1.93 (m, 2H), 1.93-1.83 (m, 2H). MS m/z: 548.15 [M+H]⁺.

### 3-Methyl-7-(1-(1-methyl-1H-indazol-5-yl)piperidin-4-yl)-5-((3-(trifluoromethyl)pyridin-2-yl)methyl)pyrido[2,3-b]pyrazin-6(5H)-one (181)

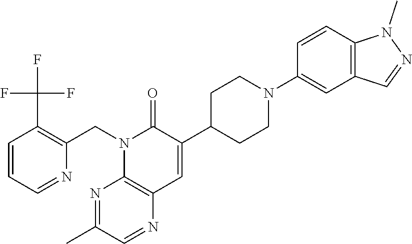 [SMILES: Cc1cnc2cc(C3CCN(c4ccc5c(cnn5C)c4)CC3)c(=O)n(Cc3ncccc3C(F)(F)F)c2n1]

Step 1: 3-methyl-7-(1-(1-methyl-1H-indazol-5-yl) piperidin-4-yl)-5-((3-(trifluoromethyl) pyridin-2-yl)methyl)pyrido[2,3-b]pyrazin-6(5H)-one. Followed the General Procedure G using 3-methyl-7-(piperidin-4-yl)-5-((3-(trifluoromethyl)pyridin-2-yl)methyl)pyrido[2,3-b]pyrazin-6(5H)-one (25 mg, 0.062 mmol, 1 equiv) and 5-bromo-1-methylindazole (19.6 mg, 0.093 mmol, 1.5 equiv) as the starting materials to give 3-methyl-7-(1-(1-methyl-1H-indazol-5-yl)piperidin-4-yl)-5-((3-(trifluoromethyl)pyridin-2-yl)methyl)pyrido[2,3-b]pyrazin-6(5H)-one (5 mg, 15.0%) as a white solid. MS m/z: 534.10 [M+H]⁺. ¹H NMR (400 MHz, Chloroform-d) δ 8.43 (d, J=4.9 Hz, 1H), 8.34 (s, 1H), 8.01-7.78 (m, 3H), 7.30 (s, 2H), 7.22 (dd, J=13.3, 8.3 Hz, 2H), 6.04 (s, 2H), 4.05 (s, 3H), 3.73 (s, 2H), 3.02 (d, J=114.2 Hz, 3H), 2.52 (s, 3H), 2.18 (s, 2H), 1.90 (s, 2H).

### 7-((1r,4r)-4-(2-Fluoro-6-methylphenyl)cyclohexyl)-2-methyl-5-((3-(trifluoromethyl)pyridin-2-yl)methyl)pyrido[2,3-b]pyrazin-6(5H)-one (182)

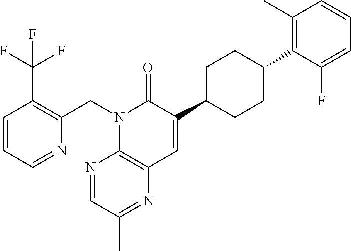 [SMILES: Cc1cnc2c(cc([C@H]3CC[C@H](c4c(C)cccc4F)CC3)c(=O)n2Cc2ncccc2C(F)(F)F)n1]

Step 1: 2-bromo-7-(4-(2-fluoro-6-methylphenyl)cyclohexyl)pyrido[2,3-b]pyrazin-6(5H)-one: Followed the General Procedure D using ethyl 2-(4-(2-fluoro-6-methylphenyl)cyclohexyl)acetate (500 mg, 1.79 mmol, 1.0 equiv.) and 3-amino-6-bromopyrazine-2-carbaldehyde (435 mg, 2.16 mmol, 1.2 equiv.) as the starting materials to give 2-bromo-7-(4-(2-fluoro-6-methylphenyl)cyclohexyl)pyrido[2,3-b]pyrazin-6(5H)-one (100 mg, 13.4%) as a light yellow oil. MS m/z: 416 [M+H]⁺.

Step 2: 7-(4-(2-fluoro-6-methylphenyl)cyclohexyl)-2-methylpyrido[2,3-b]pyrazin-6(5H)-one: Followed the General Procedure AL using ethyl 2-bromo-7-(4-(2-fluoro-6-methylphenyl)cyclohexyl)pyrido[2,3-b]pyrazin-6(5H)-one (100 mg, 0.24 mmol, 1.0 equiv.) and methylboronic acid (21.6 mg, 0.36 mmol, 1.5 equiv.) as the starting materials to give 7-(4-(2-fluoro-6-methylphenyl)cyclohexyl)-2-methylpyrido[2,3-b]pyrazin-6(5H)-one (50 mg, 59%) as a light yellow solid. MS m/z: 352 [M+H]⁺.

Step 3: 7-((1r,4r)-4-(2-fluoro-6-methylphenyl)cyclohexyl)-2-methyl-5-((3-(trifluoromethyl)pyridin-2-yl)methyl)pyrido[2,3-b]pyrazin-6(5H)-one: Followed the General Procedure E using 7-(4-(2-fluoro-6-methylphenyl)cyclohexyl)-2-methylpyrido[2,3-b]pyrazin-6(5H)-one (50.0 mg, 0.142 mmol, 1.0 equiv.) and 2-(chloromethyl)-3-(trifluoromethyl)pyridine (30.6 mg, 0.156 mmol, 1.1 equiv.) as the starting materials to give the crude product 7-(4-(2-fluoro-6-methylphenyl)cyclohexyl)-2-methyl-5-((3-(trifluoromethyl)pyridin-2-yl)methyl)pyrido[2,3-b]pyrazin-6(5H)-one (20 mg) as a light yellow solid. The crude product was purified by Prep-HPLC with the following conditions (Column: YMC-Actus Triart C18 ExRS Column, 30*150 mm, 5 m; Mobile Phase A: Water (10 mmol/L NH₄HCO₃), Mobile Phase B: ACN; Flow rate: 60 mL/min; Gradient: 70% B to 90% B in 10 min; Wave Length: 254 nm/220 nm; RT1 (min): 14.73) to afford 7-((1r,4r)-4-(2-fluoro-6-methylphenyl)cyclohexyl)-2-methyl-5-((3-(trifluoromethyl)pyridin-2-yl)methyl)pyrido[2,3-b]pyrazin-6(5H)-one (4.3 mg, 5.8%) as a white solid. ¹H NMR (400 MHz, Chloroform-d) δ 8.45-8.37 (m, 1H), 8.24 (s, 1H), 8.01-7.91 (m, 1H), 7.82 (d, J=0.9 Hz, 1H), 7.23 (dd, J=7.9, 4.9 Hz, 1H), 7.03 (td, J=7.8, 5.4 Hz, 1H), 6.92 (d, J=7.5 Hz, 1H), 6.84 (dd, J=11.7, 8.3 Hz, 1H), 6.03 (s, 2H), 3.15 (t, J=12.0 Hz, 1H), 2.90 (t, J=12.4 Hz, 1H), 2.64 (s, 3H), 2.38 (s, 3H), 2.24-2.09 (m, 4H), 1.89-1.80 (m, 2H), 1.53 (q, J=12.7 Hz, 2H). MS m/z: 511.2 [M+H]⁺.

### 3-((1r,4r)-4-(2-Fluoro-6-methylphenyl)cyclohexyl)-7-methyl-1-((3-(trifluoromethyl)pyridin-2-yl)methyl)-1,8-naphthyridin-2(1H)-one (183)

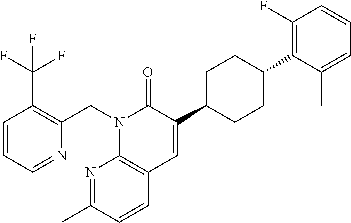 [SMILES: Cc1ccc2cc([C@H]3CC[C@H](c4c(C)cccc4F)CC3)c(=O)n(Cc3ncccc3C(F)(F)F)c2n1]

Step 1: 3-((1r,4r)-4-(2-fluoro-6-methylphenyl)cyclohexyl)-7-methyl-1-((3-(trifluoromethyl)pyridin-2-yl)methyl)-1,8-naphthyridin-2(1H)-one: Followed the General Procedure E using 3-((1r,4r)-4-(2-fluoro-6-methylphenyl)cyclohexyl)-7-methyl-1,8-naphthyridin-2(1H)-one (80 mg, 228 μmol, 1.0 equiv) and 2-(chloromethyl)-3-(trifluoromethyl)pyridine (44.5 mg, 228 μmol, 1.0 equiv) as the starting materials to give 3-((1r,4r)-4-(2-fluoro-6-methylphenyl)cyclohexyl)-7-methyl-1-((3-(trifluoromethyl)pyridin-2-yl)methyl)-1,8-naphthyridin-2(1H)-one (19.6 mg, 16.7%) as a white solid. ¹H NMR (400 MHz, Chloroform-d) δ 8.49 (d, J=4.9 Hz, 1H), 8.00 (d, J=7.9 Hz, 1H), 7.75 (d, J=7.8 Hz, 1H), 7.54 (d, J=0.8 Hz, 1H), 7.23 (s, 1H), 7.02 (dt, J=14.1, 7.8 Hz, 2H), 6.91 (d, J=7.4 Hz, 1H), 6.88-6.79 (m, 1H), 6.13 (s, 2H), 3.12 (t, J=12.2 Hz, 1H), 2.90 (t, J=12.4 Hz, 1H), 2.47 (s, 3H), 2.37 (s, 3H), 2.21-2.10 (m, 4H), 1.87-1.79 (m, 2H), 1.53 (q, J=12.5 Hz, 2H). MS m/z: 510.1 [M+H]⁺.

### 7-(1-(5-Fluoro-1-methyl-1H-indazol-4-yl)piperidin-4-yl)-3-methyl-5-((3-(trifluoromethyl)pyridin-2-yl)methyl)pyrido[2,3-b]pyrazin-6(5H)-one (184)

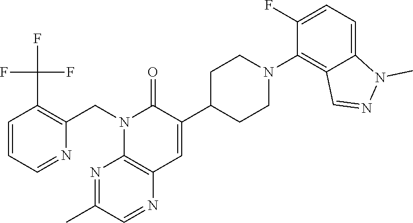 [SMILES: Cc1cnc2cc(C3CCN(c4c(F)ccc5c4cnn5C)CC3)c(=O)n(Cc3ncccc3C(F)(F)F)c2n1]

Step 1: 7-(1-(5-fluoro-1-methyl-1H-indazol-4-yl)piperidin-4-yl)-3-methyl-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (40 mg, 0.183 mmol, 1 equiv) and 4-bromo-5-fluoro-1-methyl-1H-indazole (88.4 mg, 0.220 mmol, 1.2 equiv) as the starting materials to give 7-(1-(5-fluoro-1-methyl-1H-indazol-4-yl)piperidin-4-yl)-3-methyl-5-((3-(trifluoromethyl)pyridin-2-yl)methyl)pyrido[2,3-b]pyrazin-6(5H)-one (16.5 mg, 14.7%) as a white solid. ¹H NMR (400 MHz, Chloroform-d) δ 8.50-8.29 (m, 3H), 8.01-7.94 (m, 1H), 7.90 (s, 1H), 7.25-7.21 (m, 1H), 7.21-7.11 (m, 1H), 7.01 (d, J=8.8 Hz, 1H), 6.04 (s, 2H), 4.04 (s, 3H), 3.82 (d, J=12.0 Hz, 2H), 3.50 (s, 2H), 3.28 (s, 1H), 2.52 (s, 3H), 2.17 (s, 4H). MS m/z: 552.1 [M+H]⁺.

### 7-(1-(1,5-Dimethyl-1H-indazol-4-yl)piperidin-4-yl)-3-methyl-5-((3-(trifluoromethyl)pyridin-2-yl)methyl)pyrido[2,3-b]pyrazin-6(5H)-one (185)

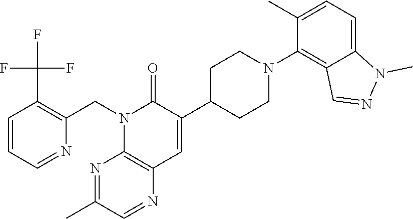 [SMILES: Cc1cnc2cc(C3CCN(c4c(C)ccc5c4cnn5C)CC3)c(=O)n(Cc3ncccc3C(F)(F)F)c2n1]

Step 1: 7-(1-(1,5-dimethyl-1H-indazol-4-yl)piperidin-4-yl)-3-methyl-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (45 mg, 0.112 mmol, 1 equiv.) and 4-bromo-1,5-dimethyl-1H-indazole (32.6 mg, 0.146 mmol, 1.3 equiv.) as the starting materials to give 7-(1-(1,5-dimethyl-1H-indazol-4-yl)piperidin-4-yl)-3-methyl-5-((3-(trifluoromethyl)pyridin-2-yl)methyl)pyrido[2,3-b]pyrazin-6(5H)-one (11.3 mg, 16.8%) as a white solid. ¹H NMR (400 MHz, Chloroform-d) δ 8.43 (dd, J=5.0, 1.6 Hz, 1H), 8.35 (s, 1H), 8.13 (s, 1H), 8.01-7.90 (m, 2H), 7.23 (d, J=7.6 Hz, 2H), 7.12 (s, 1H), 6.05 (s, 2H), 4.04 (s, 3H), 3.54 (s, 4H), 3.30 (s, 1H), 2.52 (s, 6H), 2.24-2.00 (m, 4H). MS m/z: 548.15 [M+H]⁺.

### 3-Methyl-7-(1-(1-methyl-1H-indazol-3-yl)piperidin-4-yl)-5-((3-(trifluoromethyl)pyridin-2-yl)methyl)pyrido[2,3-b]pyrazin-6(5H)-one (186)

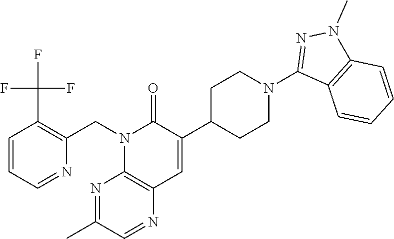 [SMILES: Cc1cnc2cc(C3CCN(c4nn(C)c5ccccc45)CC3)c(=O)n(Cc3ncccc3C(F)(F)F)c2n1]

Step 1: 3-methyl-7-(1-(1-methyl-1H-indazol-3-yl)piperidin-4-yl)-5-((3-(trifluoromethyl)pyridin-2-yl)methyl)pyrido[2,3-b]pyrazin-6(5H)-one: Followed the General Procedure G using 3-methyl-7-(piperidin-4-yl)-5-((3-(trifluoromethyl)pyridin-2-yl)methyl)pyrido[2,3-b]pyrazin-6(5H)-one (70 mg, 0.174 mmol, 1 equiv) and 3-bromo-1-methylindazole (54.9 mg, 0.261 mmol, 1.5 equiv) as the starting materials to give 3-methyl-7-(1-(1-methyl-1H-indazol-3-yl)piperidin-4-yl)-5-((3-(trifluoromethyl)pyridin-2-yl)methyl)pyrido[2,3-b]pyrazin-6(5H)-one (24.4 mg, 25.5%) as a white solid. ¹H NMR (400 MHz, Chloroform-d) δ 8.43 (d, J=4.9 Hz, 1H), 8.33 (s, 1H), 7.97 (d, J=7.9 Hz, 1H), 7.86 (s, 1H), 7.72 (d, J=30.4 Hz, 1H), 7.34 (t, J=7.7 Hz, 1H), 7.23 (q, J=5.0 Hz, 2H), 7.03 (t, J=7.3 Hz, 1H), 6.04 (s, 2H), 4.08 (d, J=12.2 Hz, 2H), 3.91 (s, 3H), 3.30-3.05 (m, 3H), 2.51 (s, 3H), 2.16 (d, J=12.4 Hz, 2H), 1.99 (d, J=13.2 Hz, 2H). MS m/z: 534.1[M+H]⁺.

### 5-((3-(Difluoromethyl)pyridin-2-yl)methyl)-7-((1S,3R)-3-(2-fluoro-6-methylphenyl)cyclopentyl)-3-methylpyrido[2,3-b]pyrazin-6(5H)-one (187A); 5-((3-(Difluoromethyl)pyridin-2-yl)methyl)-7-((1S,3S)-3-(2-fluoro-6-methylphenyl)cyclopentyl)-3-methylpyrido[2,3-b]pyrazin-6(5H)-one (187B); 5-((3-(Difluoromethyl)pyridin-2-yl)methyl)-7-((1R,3S)-3-(2-fluoro-6-methylphenyl)cyclopentyl)-3-methylpyrido[2,3-b]pyrazin-6(5H)-one (187C); 5-((3-(Difluoromethyl)pyridin-2-yl)methyl)-7-((1R,3R)-3-(2-fluoro-6-methylphenyl)cyclopentyl)-3-methylpyrido[2,3-b]pyrazin-6(5H)-one (187D)

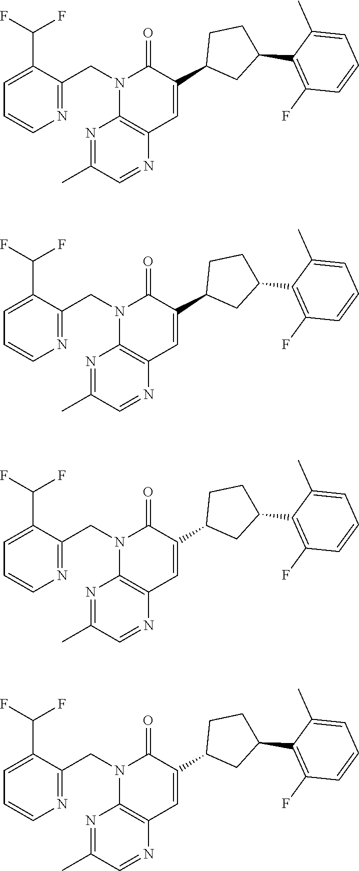 [SMILES: Cc1cnc2cc([C@H]3CC[C@@H](c4c(C)cccc4F)C3)c(=O)n(Cc3ncccc3C(F)F)c2n1 ; Cc1cnc2cc([C@H]3CC[C@H](c4c(C)cccc4F)C3)c(=O)n(Cc3ncccc3C(F)F)c2n1 ; Cc1cnc2cc([C@@H]3CC[C@H](c4c(C)cccc4F)C3)c(=O)n(Cc3ncccc3C(F)F)c2n1 ; Cc1cnc2cc([C@@H]3CC[C@@H](c4c(C)cccc4F)C3)c(=O)n(Cc3ncccc3C(F)F)c2n1]

Step 1: 5-((3-(difluoromethyl)pyridin-2-yl)methyl)-7-(3-(2-fluoro-6-methylphenyl)cyclopentyl)-3-methylpyrido[2,3-b]pyrazin-6(5H)-one: Followed the General Procedure E using 7-(3-(2-fluoro-6-methylphenyl)cyclopentyl)-3-methylpyrido[2,3-b]pyrazin-6(5H)-one (210 mg, 0.622 mmol, 1 equiv) and 2-(chloromethyl)-3-(difluoromethyl)pyridine (132 mg, 0.746 mmol, 1.2 equiv) as the starting materials to give the crude product 5-((3-(difluoromethyl)pyridin-2-yl)methyl)-7-(3-(2-fluoro-6-methylphenyl)cyclopentyl)-3-methylpyrido[2,3-b]pyrazin-6(5H)-one (160 mg). The crude product was purified by Chiral-HPLC with the following conditions (Column: CHIRAL ART Cellulose-SZ, 3*25 cm, 5 m; Mobile Phase A: Hex (10 mM NH₃-MeOH), Mobile Phase B: IPA-HPLC; Flow rate: 40 mL/min; Gradient: isocratic 50; Wave Length: 220/260 nm; RT1 (min): 7.0775; RT2 (min): 7.86; RT3 (min):8.84; RT4 (min): 16.635; Sample Solvent: IPA-HPLC; Injection Volume: 0.2 mL; Number Of Runs: 17) to afford 5-((3-(difluoromethyl)pyridin-2-yl)methyl)-7-((1S,3R)-3-(2-fluoro-6-methylphenyl)cyclopentyl)-3-methylpyrido[2,3-b]pyrazin-6(5H)-one (9.6 mg, 5.9%), 5-((3-(difluoromethyl)pyridin-2-yl)methyl)-7-((1S,3S)-3-(2-fluoro-6-methylphenyl)cyclopentyl)-3-methylpyrido[2,3-b]pyrazin-6(5H)-one (11.2 mg, 6.9%), 5-((3-(difluoromethyl)pyridin-2-yl)methyl)-7-((1R,3S)-3-(2-fluoro-6-methylphenyl)cyclopentyl)-3-methylpyrido[2,3-b]pyrazin-6(5H)-one (5.4 mg, 3.1%) and 5-((3-(difluoromethyl)pyridin-2-yl)methyl)-7-((1R,3R)-3-(2-fluoro-6-methylphenyl)cyclopentyl)-3-methylpyrido[2,3-b]pyrazin-6(5H)-one (42.4 mg, 26.2%).

187A: 5-((3-(difluoromethyl)pyridin-2-yl)methyl)-7-((1S,3R)-3-(2-fluoro-6-methylphenyl)cyclopentyl)-3-methylpyrido[2,3-b]pyrazin-6(5H)-one. ¹H NMR (400 MHz, Chloroform-d) δ 8.50 (d, J=4.9 Hz, 1H), 8.34 (s, 1H), 8.01 (d, J=7.8 Hz, 1H), 7.88 (s, 1H), 7.37-7.29 (m, 1H), 7.13 (s, 1H), 7.08-6.98 (m, 1H), 6.96-6.81 (m, 2H), 5.93 (s, 2H), 3.84-3.71 (m, 1H), 3.63-3.49 (m, 1H), 2.57 (s, 3H), 2.50-2.42 (m, 1H), 2.36 (s, 3H), 2.34-2.26 (m, 1H), 2.14-1.99 (m, 3H), 1.93-1.82 (m, 1H). MS m/z: 479.1 [M+H]⁺.

187B: 5-((3-(difluoromethyl)pyridin-2-yl)methyl)-7-((1S,3S)-3-(2-fluoro-6-methylphenyl)cyclopentyl)-3-methylpyrido[2,3-b]pyrazin-6(5H)-one. ¹H NMR (400 MHz, Chloroform-d) δ 8.48 (d, J=4.9 Hz, 1H), 8.35 (s, 1H), 8.01-7.92 (m, 2H), 7.34-7.26 (m, 2H), 7.08-7.00 (m, 1H), 6.94-6.83 (m, 2H), 5.91 (s, 2H), 3.58-3.44 (m, 2H), 2.56 (s, 3H), 2.34 (s, 4H), 2.30-2.21 (m, 1H), 2.16-2.08 (m, 2H), 2.05-1.95 (m, 2H). MS m/z: 479.1 [M+H]⁺.

187C: 5-((3-(difluoromethyl)pyridin-2-yl)methyl)-7-((1R,3S)-3-(2-fluoro-6-methylphenyl)cyclopentyl)-3-methylpyrido[2,3-b]pyrazin-6(5H)-one. ¹H NMR (400 MHz, Chloroform-d) δ 8.51 (d, J=4.3 Hz, 1H), 8.34 (d, J=5.2 Hz, 1H), 8.02 (d, J=7.8 Hz, 1H), 7.88 (s, 1H), 7.44-7.27 (m, 2H), 7.08-6.98 (m, 1H), 6.93-6.82 (m, 2H), 5.93 (s, 2H), 3.84-3.71 (m, 1H), 3.60-3.47 (m, 1H), 2.57 (s, 3H), 2.50-2.40 (m, 1H), 2.36 (s, 4H), 2.16-2.02 (m, 3H), 1.95-1.78 (m, 1H). MS m/z: 479.1 [M+H]⁺.

187D: 5-((3-(difluoromethyl)pyridin-2-yl)methyl)-7-((1R,3R)-3-(2-fluoro-6-methylphenyl)cyclopentyl)-3-methylpyrido[2,3-b]pyrazin-6(5H)-one. ¹H NMR (400 MHz, Chloroform-d) δ 8.45 (d, J=4.9 Hz, 1H), 8.34 (s, 1H), 7.93 (d, J=4.7 Hz, 2H), 7.28 (s, 1H), 7.24 (d, J=5.1 Hz, 1H), 7.09-6.99 (m, 1H), 6.94-6.82 (m, 2H), 5.88 (s, 2H), 3.51 (p, J=8.2 Hz, 2H), 2.55 (s, 3H), 2.41-2.31 (m, 4H), 2.28-2.23 (m, 1H), 2.20-2.07 (m, 2H), 2.07-1.94 (m, 2H). MS m/z: 479.1 [M+H]⁺.

### 7-((1r,4r)-4-(4-(1,1-Difluoroethyl)pyridin-3-yl)cyclohexyl)-3-methyl-5-((3-(trifluoromethyl)pyridin-2-yl)methyl)pyrido[2,3-b]pyrazin-6(5H)-one (188)

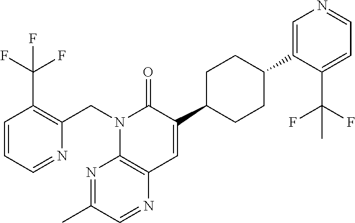 [SMILES: Cc1cnc2cc([C@H]3CC[C@H](c4cnccc4C(C)(F)F)CC3)c(=O)n(Cc3ncccc3C(F)(F)F)c2n1]

Step 1: 3-chloro-4-(1,1-difluoroethyl)pyridine: Followed the General Procedure AE using 1-(3-chloropyridin-4-yl)ethan-1-one (1 g, 6.45 mmol, 1 equiv) as the starting material to give 3-chloro-4-(1,1-difluoroethyl)pyridine (800 mg, 70%) as a colorless oil. MS m/z: 178 [M+H]⁺.

Step 2: ethyl 2-(4-(4-(1,1-difluoroethyl)pyridin-3-yl)cyclohex-3-en-1-yl)acetate: Followed the General Procedure AL using 3-chloro-4-(1,1-difluoroethyl)pyridine (800 mg, 4.51 mmol, 1 equiv) and ethyl 2-(4-(4,4,5,5-tetramethyl-1,3,2-dioxaborolan-2-yl)cyclohex-3-en-1-yl)acetate (1.59 g, 5.42 mmol, 1.2 equiv.) as the starting materials to give ethyl 2-(4-(4-(1,1-difluoroethyl)pyridin-3-yl)cyclohex-3-en-1-yl)acetate (600 mg, 42%) as a colorless oil. MS m/z: 310 [M+H]⁺.

Step 3: ethyl 2-(4-(4-(1,1-difluoroethyl)pyridin-3-yl)cyclohexyl)acetate: Followed the General Procedure AR using ethyl 2-(4-(4-(1,1-difluoroethyl)pyridin-3-yl)cyclohex-3-en-1-yl)acetate (600 mg, 1.94 mmol, 1 equiv.) as the starting material to give the crude product ethyl 2-(4-(4-(1,1-difluoroethyl)pyridin-3-yl)cyclohexyl)acetate (600 mg) as a colorless oil. MS m/z: 312 [M+H]⁺.

Step 4: 7-((1r,4r)-4-(4-(1,1-difluoroethyl)pyridin-3-yl)cyclohexyl)-3-methylpyrido[2,3-b]pyrazin-6(5H)-one: Followed the General Procedure D using ethyl 2-(4-(4-(1,1-difluoroethyl)pyridin-3-yl)cyclohexyl)acetate (600 mg, 1.92 mmol, 1 equiv.) and 3-amino-5-methylpyrazine-2-carbaldehyde (317 mg, 2.31 mmol, 1.2 equiv.) as the starting materials to give 7-((1r,4r)-4-(4-(1,1-difluoroethyl)pyridin-3-yl)cyclohexyl)-3-methylpyrido[2,3-b]pyrazin-6(5H)-one (45 mg, 6%) as a white solid. MS m/z: 385 [M+H]⁺.

Step 5: 7-((1r,4r)-4-(4-(1,1-difluoroethyl)pyridin-3-yl)cyclohexyl)-3-methyl-5-((3-(trifluoromethyl)pyridin-2-yl)methyl)pyrido[2,3-b]pyrazin-6(5H)-one: Followed the General Procedure E using 7-((1r,4r)-4-(4-(1,1-difluoroethyl)pyridin-3-yl)cyclohexyl)-3-methylpyrido[2,3-b]pyrazin-6(5H)-one (45 mg, 117 μmol, 1.0 equiv.) and 2-(chloromethyl)-3-(trifluoromethyl)pyridine (27.4 mg, 140 μmol, 1.2 equiv.) as the starting materials to give 7-((1r,4r)-4-(4-(1,1-difluoroethyl)pyridin-3-yl)cyclohexyl)-3-methyl-5-((3-(trifluoromethyl)pyridin-2-yl)methyl)pyrido[2,3-b]pyrazin-6(5H)-one (5.2 mg, 8%) as a white solid. ¹H NMR (400 MHz, Chloroform-d) δ 8.75 (s, 1H), 8.57 (d, J=5.5 Hz, 1H), 8.42 (d, J=4.9 Hz, 1H), 8.33 (s, 1H), 8.01-7.94 (m, 1H), 7.81 (s, 1H), 7.63 (s, 1H), 7.25-7.21 (m, 1H), 6.03 (s, 2H), 3.20-3.08 (m, 2H), 2.51 (s, 3H), 2.23 (d, J=12.7 Hz, 2H), 2.02 (t, J=18.2 Hz, 5H), 1.81 (q, J=12.6 Hz, 2H), 1.65 (t, J=12.1 Hz, 2H). MS m/z: 544.1 [M+H]⁺.

### 7-((1r,4r)-4-(2-Fluoro-4-methoxypyridin-3-yl)cyclohexyl)-3-methyl-5-((3-(trifluoromethyl)pyridin-2-yl)methyl)pyrido[2,3-b]pyrazin-6(5H)-one (189)

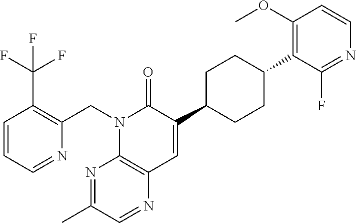 [SMILES: COc1ccnc(F)c1[C@H]1CC[C@H](c2cc3ncc(C)nc3n(Cc3ncccc3C(F)(F)F)c2=O)CC1]

Step 1: ethyl 2-(4-(2-fluoro-4-methoxypyridin-3-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (370 mg, 1.26 mmol, 1 equiv.) and 3-bromo-2-fluoro-4-methoxypyridine (311 mg, 1.51 mmol, 1.2 equiv.) as the starting materials to give ethyl 2-(4-(2-fluoro-4-methoxypyridin-3-yl)cyclohex-3-en-1-yl)acetate (310 mg, 84%) as a light yellow oil. MS m/z: 294 [M+H]⁺.

Step 2: ethyl 2-(4-(2-fluoro-4-methoxypyridin-3-yl)cyclohexyl)acetate: Followed the General Procedure AR using ethyl 2-(4-(2-fluoro-4-methoxypyridin-3-yl)cyclohex-3-en-1-yl)acetate (310 mg, 1.06 mmol, 1 equiv.) as the starting material to give the crude product ethyl 2-(4-(2-fluoro-4-methoxypyridin-3-yl)cyclohexyl)acetate (260 mg) as a colorless oil. MS m/z: 296 [M+H]⁺.

Step 3: 7-((1r,4r)-4-(2-fluoro-4-methoxypyridin-3-yl)cyclohexyl)-3-methylpyrido[2,3-b]pyrazin-6(5H)-one: Followed the General Procedure D using 2-(4-(2-fluoro-4-methoxypyridin-3-yl)cyclohexyl)acetate (260 mg, 0.878 mmol, 1 equiv.) and 3-amino-5-methylpyrazine-2-carbaldehyde (182 mg, 1.32 mmol, 1.5 equiv.) as the starting materials to give 7-((1r,4r)-4-(2-fluoro-4-methoxypyridin-3-yl)cyclohexyl)-3-methylpyrido[2,3-b]pyrazin-6(5H)-one (50 mg, 15.4%) as an off-white solid. MS m/z: 369 [M+H]⁺.

Step 4: 7-((1r,4r)-4-(2-fluoro-4-methoxypyridin-3-yl)cyclohexyl)-3-methyl-5-((3-(trifluoromethyl)pyridin-2-yl)methyl)pyrido[2,3-b]pyrazin-6(5H)-one: Followed the General Procedure E using 7-((1r,4r)-4-(2-fluoro-4-methoxypyridin-3-yl)cyclohexyl)-3-methylpyrido[2,3-b]pyrazin-6(5H)-one (50 mg, 0.136 mmol, 1 equiv.) and 2-(bromomethyl)-3-(trifluoromethyl)pyridine hydrobromide (65 mg, 0.204 mmol, 1.5 equiv.) as the starting materials to give 7-((1r,4r)-4-(2-fluoro-4-methoxypyridin-3-yl)cyclohexyl)-3-methyl-5-((3-(trifluoromethyl)pyridin-2-yl)methyl)pyrido[2,3-b]pyrazin-6(5H)-one (18.1 mg, 24.7%) as a white solid. ¹H NMR (400 MHz, Chloroform-d) δ 8.46 (d, J=4.8 Hz, 1H), 8.32 (s, 1H), 8.06-7.99 (m, 2H), 7.91 (s, 1H), 7.32-7.27 (m, 1H), 6.76 (d, J=5.6 Hz, 1H), 6.05 (s, 2H), 3.94 (s, 3H), 3.21-3.05 (m, 2H), 2.55 (s, 3H), 2.19-2.05 (m, 4H), 1.85-1.74 (m, 2H), 1.55 (q, J=12.7 Hz, 2H). MS m/z: 528.05 [M+H]⁺.

### 3-Methyl-5-((3-(trifluoromethyl)pyridin-2-yl)methyl)-7-((1r,4r)-4-(1,3,5-trimethyl-1H-pyrazol-4-yl)cyclohexyl)pyrido[2,3-b]pyrazin-6(5H)-one (190)

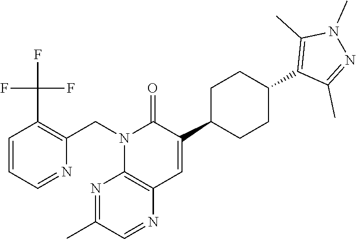 [SMILES: Cc1cnc2cc([C@H]3CC[C@H](c4c(C)nn(C)c4C)CC3)c(=O)n(Cc3ncccc3C(F)(F)F)c2n1]

Step 1: ethyl 2-[4-(1,3,5-trimethylpyrazol-4-yl)cyclohex-3-en-1-yl]acetate: Followed the General Procedure AL using 4-bromo-1,3,5-trimethylpyrazole (500 mg, 2.64 mmol, 1 equiv) and ethyl 2-(4-(4,4,5,5-tetramethyl-1,3,2-dioxaborolan-2-yl)cyclohex-3-en-1-yl)acetate (778 mg, 2.64 mmol, 1.0 equiv) as the starting materials to give ethyl 2-(4-(1,3,5-trimethyl-1H-pyrazol-4-yl)cyclohex-3-en-1-yl)acetate (400 mg, 54%) as a brown oil. MS m/z: 277 [M+H]⁺.

Step 2: ethyl 2-[4-(1,3,5-trimethylpyrazol-4-yl)cyclohexyl]acetate: Followed the General Procedure AR using ethyl 2-(4-(1,3,5-trimethyl-1H-pyrazol-4-yl)cyclohex-3-en-1-yl)acetate (500 mg, 1.809 mmol, 1.00 equiv) as the starting material to give the crude product ethyl 2-(4-(1,3,5-trimethyl-1H-pyrazol-4-yl)cyclohexyl)acetate (500 mg) as a colorless oil. MS m/z: 279 [M+H]⁺.

Step 3: 3-methyl-7-((1r,4r)-4-(1,3,5-trimethyl-1H-pyrazol-4-yl)cyclohexyl)pyrido[2,3-b]pyrazin-6(5H)-one: Followed the General Procedure D using ethyl 2-(4-(1,3,5-trimethyl-1H-pyrazol-4-yl)cyclohexyl)acetate (480 mg, 1.72 mmol, 1 equiv) and 3-amino-5-methylpyrazine-2-carbaldehyde (282 mg, 2.06 mmol, 1.2 equiv) as the starting materials to give 3-methyl-7-((1r,4r)-4-(1,3,5-trimethyl-1H-pyrazol-4-yl)cyclohexyl)pyrido[2,3-b]pyrazin-6(5H)-one (50 mg, 8%) as a yellow solid, MS m/z: 370 [M+H]⁺.

Step 4: 3-methyl-5-((3-(trifluoromethyl)pyridin-2-yl)methyl)-7-((1r,4r)-4-(1,3,5-trimethyl-1H-pyrazol-4-yl)cyclohexyl)pyrido[2,3-b]pyrazin-6(5H)-one. Followed the General Procedure E using 3-methyl-7-((1r,4r)-4-(1,3,5-trimethyl-1H-pyrazol-4-yl)cyclohexyl)pyrido[2,3-b]pyrazin-6(5H)-one (50 mg, 0.142 mmol, 1 equiv) and 2-(chloromethyl)-3-(trifluoromethyl)pyridine hydrochloride (39.5 mg, 0.17 mmol, 1.2 equiv) as the starting materials to give 3-methyl-5-((3-(trifluoromethyl)pyridin-2-yl)methyl)-7-((1r,4r)-4-(1,3,5-trimethyl-1H-pyrazol-4-yl)cyclohexyl)pyrido[2,3-b]pyrazin-6(5H)-one (5.4 mg, 7.4%) as a white solid. ¹H NMR (400 MHz, Chloroform-d) δ 8.42 (d, J=4.9 Hz, 1H), 8.33 (s, 1H), 7.97 (d, J=7.9 Hz, 1H), 7.79 (s, 1H), 7.24-7.19 (m, 1H), 6.03 (s, 2H), 3.88 (s, 3H), 3.06 (t, J=11.9 Hz, 1H), 2.52 (s, 4H), 2.36 (s, 3H), 2.27 (s, 3H), 2.14 (d, J=12.7 Hz, 2H), 1.82 (dd, J=12.3, 8.5 Hz, 4H), 1.61-1.58 (m, 2H). MS m/z: 511.2 [M+H]⁺.

### 3-Methyl-7-((1r,4r)-4-(1-methyl-1H-indazol-3-yl)cyclohexyl)-5-((3-(trifluoromethyl)pyridin-2-yl)methyl)pyrido[2,3-b]pyrazin-6(5H)-one (191)

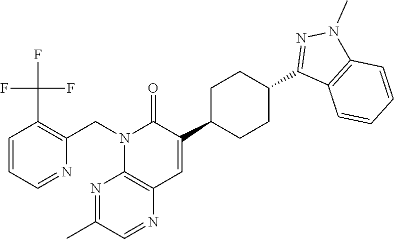 [SMILES: Cc1cnc2cc([C@H]3CC[C@H](c4nn(C)c5ccccc54)CC3)c(=O)n(Cc3ncccc3C(F)(F)F)c2n1]

Step 1: ethyl 2-(4-(1-methyl-1H-indazol-3-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (1 g, 3.4 mmol, 1 equiv) and 3-bromo-1-methylindazole (0.86 g, 4.08 mmol, 1.2 equiv) as the starting materials to give ethyl 2-(4-(1-methyl-1H-indazol-3-yl)cyclohex-3-en-1-yl)acetate (610 mg, 60%) as a yellow oil. MS m/z: 299 [M+H]⁺.

Step 2: ethyl 2-(4-(1-methyl-1H-indazol-3-yl)cyclohexyl)acetate: Followed the General Procedure AR using ethyl 2-(4-(1-methyl-1H-indazol-3-yl)cyclohex-3-en-1-yl)acetate (610 mg, 2.044 mmol, 1 equiv) as the starting material to give the crude product ethyl 2-(4-(1-methyl-1H-indazol-3-yl)cyclohexyl)acetate (500 mg) as a yellow oil. MS m/z: 301 [M+H]⁺.

Step 3: 3-methyl-7-((1r,4r)-4-(1-methyl-1H-indazol-3-yl)cyclohexyl)pyrido[2,3-b]pyrazin-6(5H)-one: Followed the General Procedure D using ethyl 2-(4-(1-methyl-1H-indazol-3-yl)cyclohexyl)acetate (500 mg, 1.664 mmol, 1 equiv) and 3-amino-5-methylpyrazine-2-carbaldehyde (273 mg, 1.99 mmol, 1.2 equiv) as the starting material to give the crude product 3-methyl-7-(4-(1-methyl-1H-indazol-3-yl)cyclohexyl)pyrido[2,3-b]pyrazin-6(5H)-one (350 mg). The crude product was purified by Prep-HPLC with the following conditions (Column: CHIRAL ART Cellulose-SC, 3*25 cm, 5 m; Mobile Phase A: Hex (10 mM NH₃-MeOH), Mobile Phase B: ACN:EtOH=2:1; Flow rate: 40 mL/min; Gradient: isocratic 50; Wave Length: 201/343 nm; RT1 (min): 8.04; RT2 (min): 9.325; Sample Solvent: MeOH:DCM=1:1-HPLC; Injection Volume: 0.4 mL; Number Of Runs: 6) to afford 3-methyl-7-((1r,4r)-4-(1-methyl-1H-indazol-3-yl)cyclohexyl)pyrido[2,3-b]pyrazin-6(5H)-one (100 mg, 34%) as a white solid.

Step 4: 3-methyl-7-((1r,4r)-4-(1-methyl-1H-indazol-3-yl)cyclohexyl)-5-((3-(trifluoromethyl)pyridin-2-yl)methyl)pyrido[2,3-b]pyrazin-6(5H)-one: Followed the General Procedure E using 3-methyl-7-((1r,4r)-4-(1-methyl-1H-indazol-3-yl)cyclohexyl)pyrido[2,3-b]pyrazin-6(5H)-one (100 mg, 0.268 mmol, 1 equiv) and 2-(chloromethyl)-3-(trifluoromethyl)pyridine (52.3 mg, 0.268 mmol, 1 equiv) as the starting materials to give 3-methyl-7-((1r,4r)-4-(1-methyl-1H-indazol-3-yl)cyclohexyl)-5-((3-(trifluoromethyl)pyridin-2-yl)methyl)pyrido[2,3-b]pyrazin-6(5H)-one (47.5 mg, 34.5%) as a white solid. ¹H NMR (400 MHz, Chloroform-d) δ 8.47-8.41 (m, 1H), 8.33 (s, 1H), 8.00-7.93 (m, 1H), 7.84 (d, J=0.9 Hz, 1H), 7.82-7.75 (m, 1H), 7.43-7.31 (m, 2H), 7.25-7.20 (m, 1H), 7.16-7.07 (m, 1H), 6.04 (s, 2H), 4.03 (s, 3H), 3.25-3.12 (m, 2H), 2.51 (s, 3H), 2.28-2.18 (m, 4H), 2.08-1.98 (m, 2H), 1.72-1.57 (m, 2H). MS m/z: 533.15 [M+H]⁺.

### 3-Methyl-7-((1r,4r)-4-(1-methyl-1H-indazol-5-yl)cyclohexyl)-5-((3-(trifluoromethyl)pyridin-2-yl)methyl)pyrido[2,3-b]pyrazin-6(5H)-one (192)

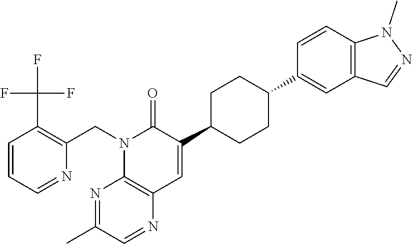 [SMILES: Cc1cnc2cc([C@H]3CC[C@H](c4ccc5c(cnn5C)c4)CC3)c(=O)n(Cc3ncccc3C(F)(F)F)c2n1]

Step 1: ethyl 2-(4-(1-methyl-1H-indazol-5-yl)cyclohex-3-en-1-yl)acetate: Followed the General Procedure AL using 5-bromo-1-methyl-1H-indazole (500 mg, 2.37 mmol, 1 equiv) and ethyl 2-(4-(4,4,5,5-tetramethyl-1,3,2-dioxaborolan-2-yl)cyclohex-3-en-1-yl)acetate (1.05 g, 3.55 mmol, 1.2 equiv) as the starting materials to give ethyl 2-(4-(1-methyl-1H-indazol-5-yl)cyclohex-3-en-1-yl)acetate (300 mg, 44%) as a white solid. MS m/z: 299 [M+H]⁺.

Step 2: ethyl 2-(4-(1-methyl-1H-indazol-5-yl)cyclohexyl)acetate: Followed the General Procedure AR using ethyl 2-(4-(1-methyl-1H-indazol-5-yl)cyclohex-3-en-1-yl)acetate (300 mg, 1 mmol, 1 equiv) as the starting material to give the crude product ethyl 2-(4-(1-methyl-1H-indazol-5-yl)cyclohexyl)acetate (250 mg) as a white solid. MS m/z: 301 [M+H]⁺.

Step 3: 3-methyl-7-(4-(1-methyl-1H-indazol-5-yl)cyclohexyl)pyrido[2,3-b]pyrazin-6(5H)-one: Followed the General Procedure D using ethyl 2-(4-(1-methyl-1H-indazol-5-yl)cyclohexyl)acetate (250 mg, 0.832 mmol, 1 quiv) and 3-amino-5-methylpyrazine-2-carbaldehyde (137 mg, 0.998 mmol, 1.2 equiv) as the starting material to give 3-methyl-7-(4-(1-methyl-1H-indazol-5-yl)cyclohexyl)pyrido[2,3-b]pyrazin-6(5H)-one (70 mg, 22%) as a white solid. MS m/z: 374 [M+H]⁺.

Step 4: 3-methyl-7-((1r,4r)-4-(1-methyl-1H-indazol-5-yl)cyclohexyl)-5-((3-(trifluoromethyl)pyridin-2-yl)methyl)pyrido[2,3-b]pyrazin-6(5H)-one: Followed the General Procedure E using 3-methyl-7-(4-(1-methyl-1H-indazol-5-yl)cyclohexyl)pyrido[2,3-b]pyrazin-6(5H)-one (70 mg, 0.187 mmol, 1 equiv) and 2-(bromomethyl)-3-(trifluoromethyl)pyridine hydrobromide (54 mg, 0.224 mmol, 1.2 equiv) as the starting materials to give 3-methyl-7-((1r,4r)-4-(1-methyl-1H-indazol-5-yl)cyclohexyl)-5-((3-(trifluoromethyl)pyridin-2-yl)methyl)pyrido[2,3-b]pyrazin-6(5H)-one (42.9 mg, 42.6%) as a white solid. ¹H NMR (300 MHz, Chloroform-d) δ 8.42 (d, J=4.8 Hz, 1H), 8.29 (s, 1H), 7.98 (d, J=8.0 Hz, 2H), 7.86 (s, 1H), 7.67 (s, 1H), 7.46-7.31 (m, 2H), 7.22 (s, 1H), 6.02 (s, 2H), 4.10 (s, 3H), 3.29 (s, 1H), 3.07 (s, 1H), 2.50 (s, 3H), 2.14 (s, 4H), 2.02 (q, J=3.8 Hz, 2H), 1.94-1.82 (m, 2H). MS m/z: 533.15 [M+H]⁺.

### 1-((3-(Difluoromethyl)pyridin-2-yl)methyl)-3-((1r,4r)-4-(2-fluoro-6-methylphenyl)cyclohexyl)-7-methyl-1,8-naphthyridin-2(1H)-one (193)

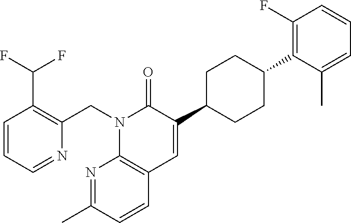 [SMILES: Cc1ccc2cc([C@H]3CC[C@H](c4c(C)cccc4F)CC3)c(=O)n(Cc3ncccc3C(F)F)c2n1]

Step 1: 1-((3-(difluoromethyl)pyridin-2-yl)methyl)-3-((1r,4r)-4-(2-fluoro-6-methylphenyl)cyclohexyl)-7-methyl-1,8-naphthyridin-2(1H)-one: Followed the General Procedure E using 3-((1r,4r)-4-(2-fluoro-6-methylphenyl)cyclohexyl)-7-methyl-1,8-naphthyridin-2(1H)-one (30 mg, 85 μmol, 1 equiv) and 2-(bromomethyl)-3-(difluoromethyl)pyridine (22.8 mg, 102 μmol, 1.2 equiv) as the starting materials to give 1-((3-(difluoromethyl)pyridin-2-yl)methyl)-3-((1r,4r)-4-(2-fluoro-6-methylphenyl)cyclohexyl)-7-methyl-1,8-naphthyridin-2(1H)-one (15 mg, 35%) as a white solid. ¹H NMR (400 MHz, Chloroform-d) δ 8.54 (d, 1H), 8.00 (d, J=7.7 Hz, 1H), 7.74 (d, J=7.8 Hz, 1H), 7.51 (s, 1H), 7.39-7.26 (m, 2H), 7.06-6.98 (m, 2H), 6.92-6.81 (m, 2H), 5.98 (s, 2H), 3.11 (t, J=12.2 Hz, 1H), 2.94-2.84 (m, 1H), 2.52 (s, 3H), 2.37 (s, 3H), 2.20-2.09 (m, 4H), 1.83 (d, J=12.7 Hz, 2H), 1.50 (q, J=12.1 Hz, 2H). MS m/z: 492.1 [M+H]⁺.

### 3-((1r,4r)-4-(2-Fluoro-6-methylphenyl)cyclohexyl)-7-methyl-1-((3-(trifluoromethyl)pyrazin-2-yl)methyl)-1,8-naphthyridin-2(1H)-one (194)

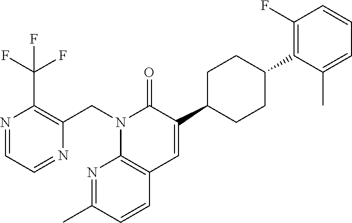 [SMILES: Cc1ccc2cc([C@H]3CC[C@H](c4c(C)cccc4F)CC3)c(=O)n(Cc3nccnc3C(F)(F)F)c2n1]

Step 1: 3-((1r,4r)-4-(2-fluoro-6-methylphenyl)cyclohexyl)-7-methyl-1-((3-(trifluoromethyl)pyrazin-2-yl)methyl)-1,8-naphthyridin-2(1H)-one: Followed the General Procedure E using 3-((1r,4r)-4-(2-fluoro-6-methylphenyl)cyclohexyl)-7-methyl-1,8-naphthyridin-2(1H)-one (40 mg, 114 μmol, 1 equiv) and 2-(bromomethyl)-3-(trifluoromethyl)pyrazine (27.4 mg, 114 μmol, 1 equiv) as the starting materials to give 3-((1r,4r)-4-(2-fluoro-6-methylphenyl)cyclohexyl)-7-methyl-1-((3-(trifluoromethyl)pyrazin-2-yl)methyl)-1,8-naphthyridin-2(1H)-one (19.6 mg, 33.4%) as a white solid. ¹H NMR (400 MHz, Methanol-d4) δ 8.57-8.48 (m, 2H), 7.98 (d, J=7.8 Hz, 1H), 7.83 (s, 1H), 7.13 (d, J=7.9 Hz, 1H), 7.05 (td, J=7.9, 5.6 Hz, 1H), 6.95 (d, J=7.6 Hz, 1H), 6.84 (dd, J=12.0, 8.0 Hz, 1H), 6.13 (s, 2H), 3.06-2.96 (m, 2H), 2.43 (s, 3H), 2.39 (s, 3H), 2.15-2.04 (m, 4H), 1.89-1.81 (m, 2H), 1.61 (d, J=9.3 Hz, 2H). MS m/z: 511.1 [M+H]⁺.

### 1-((3-(Difluoromethoxy)pyridin-2-yl)methyl)-3-((1r,4r)-4-(2-fluoro-6-methylphenyl)cyclohexyl)-7-methyl-1,8-naphthyridin-2(1H)-one (195)

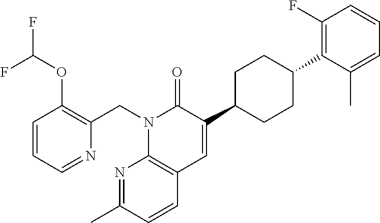 [SMILES: Cc1ccc2cc([C@H]3CC[C@H](c4c(C)cccc4F)CC3)c(=O)n(Cc3ncccc3OC(F)F)c2n1]

Step 1: 1-((3-(difluoromethoxy)pyridin-2-yl)methyl)-3-((1r,4r)-4-(2-fluoro-6-methylphenyl)cyclohexyl)-7-methyl-1,8-naphthyridin-2(1H)-one: Followed the General Procedure E using 3-((1r,4r)-4-(2-fluoro-6-methylphenyl)cyclohexyl)-7-methyl-1,8-naphthyridin-2(1H)-one (50 mg, 0.143 mmol, 1 equiv.) and 2-(chloromethyl)-3-(difluoromethoxy)pyridine (33.1 mg, 0.171 mmol, 1.2 equiv.) as the starting materials to give 1-((3-(difluoromethoxy)pyridin-2-yl)methyl)-3-((1r,4r)-4-(2-fluoro-6-methylphenyl)cyclohexyl)-7-methyl-1,8-naphthyridin-2(1H)-one (31 mg, 42.7%) as a white solid. ¹H NMR (400 MHz, Chloroform-d) δ 8.26 (d, J=4.8 Hz, 1H), 7.74 (d, J=8.0 Hz, 1H), 7.56-7.47 (m, 2H), 7.16 (m, 1H), 7.06-6.97 (m, 2H), 6.92 (t, J=7.6 Hz, 1H), 6.88-6.79 (m, 1H), 6.78-6.55 (m, 1H), 6.01 (s, 2H), 3.10 (t, J=12.4 Hz, 1H), 2.89 (t, J=12.4 Hz, 1H), 2.51 (s, 3H), 2.37 (s, 3H), 2.23-2.08 (m, 4H), 1.87-1.79 (m, 2H), 1.49 (q, J=12.4 Hz, 2H). MS m/z: 508.15 [M+H]⁺.

### 3-((1r,4r)-4-(2-Fluoro-6-methylphenyl)cyclohexyl)-7-methyl-1-((3-(trifluoromethoxy)pyridin-2-yl)methyl)-1,8-naphthyridin-2(1H)-one (196)

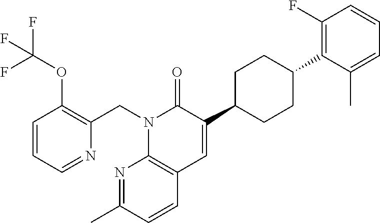 [SMILES: Cc1ccc2cc([C@H]3CC[C@H](c4c(C)cccc4F)CC3)c(=O)n(Cc3ncccc3OC(F)(F)F)c2n1]

Step 1: 3-((1r,4r)-4-(2-fluoro-6-methylphenyl)cyclohexyl)-7-methyl-1-((3-(trifluoromethoxy)pyridin-2-yl)methyl)-1,8-naphthyridin-2(1H)-one. Followed the General Procedure E using 5-((3-(difluoromethyl)pyridin-2-yl)methyl)-3-methyl-7-(piperidin-4-yl)pyrido[2,3-b]pyrazin-6(5H)-one (40 mg, 0.114 mmol, 1 equiv) and 2-(chloromethyl)-3-(trifluoromethoxy)pyridine (31.5 mg, 0.195 mmol, 1.3 equiv) as the starting materials to give 3-((1r,4r)-4-(2-fluoro-6-methylphenyl)cyclohexyl)-7-methyl-1-((3-(trifluoromethoxy)pyridin-2-yl)methyl)-1,8-naphthyridin-2(1H)-one (30 mg, 48.2%) as a white solid. ¹H NMR (300 MHz, Chloroform-d) δ 8.43 (d, J=5.1 Hz, 1H), 7.75 (d, J=7.9 Hz, 2H), 7.53 (s, 1H), 7.41-7.31 (m, 1H), 7.09-6.96 (m, 2H), 6.95-6.78 (m, 2H), 6.13 (s, 2H), 3.10 (t, J=12.3 Hz, 1H), 2.89 (s, 1H), 2.50 (s, 3H), 2.37 (s, 3H), 2.21-2.06 (m, 4H), 1.83 (d, J=12.9 Hz, 2H), 1.57-1.42 (m, 2H). MS m/z: 526.15 [M+H]⁺.

### 7-((1S,3R)-3-(2-Fluoro-6-methylphenyl)cyclopentyl)-3-methyl-5-((3-(trifluoromethyl)pyrazin-2-yl)methyl)pyrido[2,3-b]pyrazin-6(5H)-one (197A); 7-((1S,3S)-3-(2-Fluoro-6-methylphenyl)cyclopentyl)-3-methyl-5-((3-(trifluoromethyl)pyrazin-2-yl)methyl)pyrido[2,3-b]pyrazin-6(5H)-one (197B); 7-((1R,3S)-3-(2-Fluoro-6-methylphenyl)cyclopentyl)-3-methyl-5-((3-(trifluoromethyl)pyrazin-2-yl)methyl)pyrido[2,3-b]pyrazin-6(5H)-one (197C); 7-((1R,3R)-3-(2-Fluoro-6-methylphenyl)cyclopentyl)-3-methyl-5-((3-(trifluoromethyl)pyrazin-2-yl)methyl)pyrido[2,3-b]pyrazin-6(5H)-one (197D)

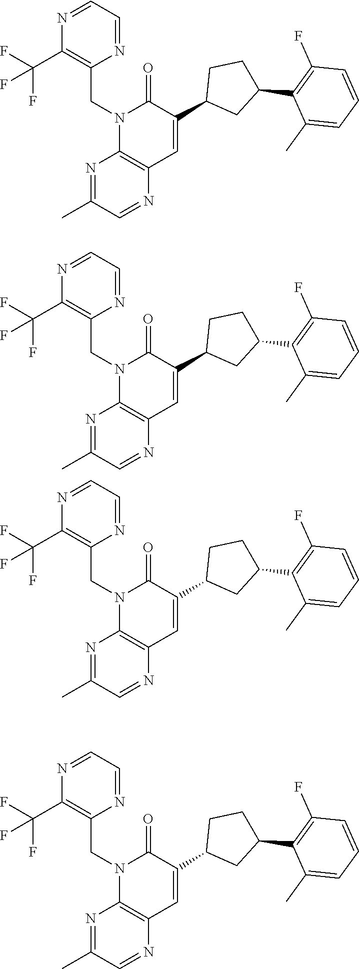

Step 1: 7-(3-(2-fluoro-6-methylphenyl)cyclopentyl)-3-methylpyrido[2,3-b]pyrazin-6(5H)-one: Followed the General Procedure D using ethyl 2-(3-(2-fluoro-6-methylphenyl)cyclopentyl)acetate (500 mg, 1.89 mmol, 1 equiv) and 3-amino-5-methylpyrazine-2-carbaldehyde (389 mg, 2.83 mmol, 1.5 equiv) as the starting materials to give 7-(3-(2-fluoro-6-methylphenyl)cyclopentyl)-3-methylpyrido[2,3-b]pyrazin-6(5H)-one (230 mg, 59%) as a yellow oil. MS m/z: 338 [M+H]⁺.

Step 2: 7-(3-(2-fluoro-6-methylphenyl)cyclopentyl)-3-methyl-5-((3-(trifluoromethyl)pyrazin-2-yl)methyl)pyrido[2,3-b]pyrazin-6(5H)-one: Followed the General Procedure E using 7-(3-(2-fluoro-6-methylphenyl)cyclopentyl)-3-methylpyrido[2,3-b]pyrazin-6(5H)-one (230 mg, 0.682 mmol, 1 equiv) and 2-(bromomethyl)-3-(trifluoromethyl)pyrazine (164 mg, 0.682 mmol, 1 equiv) as the starting materials to give the crude product 7-(3-(2-fluoro-6-methylphenyl)cyclopentyl)-3-methyl-5-((3-(trifluoromethyl)pyrazin-2-yl)methyl)pyrido[2,3-b]pyrazin-6(5H)-one (150 mg) as a white solid. The crude product was purified by Chiral-HPLC with the following conditions (Column: CHIRALPAK IG, 3*25 cm, 5 m; Mobile Phase A: Hex (10 mM NH₃-MeOH), Mobile Phase B: IPA; Flow rate: 40 mL/min; Gradient: isocratic 40; Wave Length: 200/220 nm; RT1 (min): 8.93; RT2 (min): 13.61; Sample Solvent: IPA:DCM=1:1; Injection Volume: 1 mL; Number Of Runs: 7) to afford 7-((1S,3R)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one (9.3 mg, 6.30%), with the condition (Column: CHIRALCEL OD-H, 2*25 mm, 5 m; Mobile Phase A: Hex (10 mM NH₃-MeOH), Mobile Phase B: EtOH-HPLC; Flow rate: 30 mL/min; Gradient: isocratic 2; Wave Length: 210/266 nm; RT1 (min): 12.86; RT2 (min): 15.735; RT3 (min): 16.93; Sample Solvent: EtOH:MeOH=1:2-HPLC; Injection Volume: 0.3 mL; Number Of Runs: 24) to afford 7-((1S,3S)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one (8.6 mg, 5.7%), 7-((1R,3S)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one (29.2 mg, 19.4%) and 7-((1R,3R)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one (44.9 mg, 29.9%)

197A: 7-((1S,3R)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one. ¹H NMR (300 MHz, Chloroform-d) δ 8.53-8.41 (m, 2H), 8.33 (s, 1H), 7.90 (s, 1H), 7.10-6.97 (m, 1H), 6.94-6.80 (m, 2H), 6.07 (s, 2H), 3.88-3.71 (m, 1H), 3.67-3.49 (m, 1H), 2.50 (s, 4H), 2.37 (s, 4H), 2.19-2.10 (m, 2H), 2.03 (d, J=12.0 Hz, 1H), 1.96-1.86 (m, 1H). MS m/z: 498.10 [M+H]⁺.

197B: 7-((1S,3S)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one. ¹H NMR (300 MHz, Chloroform-d) δ 8.49 (d, J=2.4 Hz, 1H), 8.44 (d, J=2.4 Hz, 1H), 8.33 (s, 1H), 7.90 (s, 1H), 7.10-6.97 (m, 1H), 6.94-6.80 (m, 2H), 6.07 (s, 2H), 3.88-3.71 (m, 1H), 3.67-3.49 (m, 1H), 2.50 (s, 4H), 2.37 (s, 4H), 2.17-2.09 (m, 2H), 2.06-2.01 (m, 1H), 1.95-1.86 (m, 1H). MS m/z: 498.10 [M+H]⁺.

197C: 7-((1R,3S)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one. ¹H NMR (300 MHz, Chloroform-d) δ 8.49 (d, J=2.4 Hz, 1H), 8.44 (d, J=2.4 Hz, 1H), 8.33 (s, 1H), 7.98 (d, J=1.1 Hz, 1H), 7.14-6.98 (m, 1H), 6.96-6.81 (m, 2H), 6.07 (s, 2H), 3.61-3.43 (m, 2H), 2.50 (s, 3H), 2.37 (s, 5H), 2.18-1.94 (m, 4H). MS m/z: 498.10 [M+H]⁺.

197D: 7-((1R,3R)-3-(2-fluoro-6-methylphenyl)cyclopentyl)-3-methyl-5-((3-(trifluoromethyl)pyrazin-2-yl)methyl)pyrido[2,3-b]pyrazin-6(5H)-one. ¹H NMR (300 MHz, Chloroform-d) δ 8.49 (s, 1H), 8.44 (s, 1H), 8.33 (s, 1H), 7.99 (s, 1H), 7.11-6.98 (m, 1H), 6.96-6.81 (m, 2H), 6.07 (s, 2H), 3.51 (d, J=9.6 Hz, 2H), 2.51 (s, 3H), 2.37 (s, 5H), 2.19-1.99 (m, 4H). MS m/z: 498.05 [M+H]⁺.

### 6-((1r,4r)-4-(2-Fluoro-6-methylphenyl)cyclohexyl)-2-methyl-8-((3-(trifluoromethoxy)pyridin-2-yl)methyl)pyrido[2,3-d]pyrimidin-7(8H)-one (198)

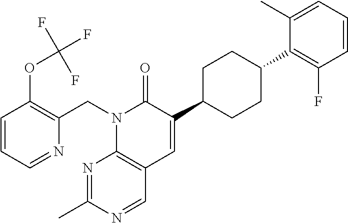 [SMILES: Cc1ncc2cc([C@H]3CC[C@H](c4c(C)cccc4F)CC3)c(=O)n(Cc3ncccc3OC(F)(F)F)c2n1]

Step 1: 4-amino-2-methylpyrimidine-5-carbaldehyde: Followed the General Procedure AM using (4-amino-2-methylpyrimidin-5-yl)methanol (500 mg, 3.593 mmol, 1 equiv) and MnO₂ (468.57 mg, 5.389 mmol, 1.5 equiv) as the starting materials to give 4-amino-2-methylpyrimidine-5-carbaldehyde (300 mg, 60%) as a yellow solid. MS m/z: 138 [M+H]⁺.

Step 2: 6-((1r,4r)-4-(2-fluoro-6-methylphenyl)cyclohexyl)-2-methylpyrido[2,3-d]pyrimidin-7(8H)-one: Followed the General Procedure D using ethyl 2-((1r,4r)-4-(2-fluoro-6-methylphenyl)cyclohexyl)acetate (406 mg, 1.45 mmol, 1.0 equiv) and 4-amino-2-methylpyrimidine-5-carbaldehyde (240 mg, 1.75 mmol, 1.2 equiv) as the starting materials to give 6-((1r,4r)-4-(2-fluoro-6-methylphenyl)cyclohexyl)-2-methylpyrido[2,3-d]pyrimidin-7(8H)-one (150 mg, 29%). MS m/z: 352 [M+H]⁺.

Step 3: 6-((1r,4r)-4-(2-fluoro-6-methylphenyl)cyclohexyl)-2-methyl-8-((3-(trifluoromethoxy)pyridin-2-yl)methyl)pyrido[2,3-d]pyrimidin-7(8H)-one: Followed the General Procedure E using 6-((1r,4r)-4-(2-fluoro-6-methylphenyl)cyclohexyl)-2-methylpyrido[2,3-d]pyrimidin-7(8H)-one (80 mg, 0.228 mmol, 1 equiv) and 2-(chloromethyl)-3-(trifluoromethoxy)pyridine (58 mg, 0.274 mmol, 1.2 equiv) as the starting materials to give 6-((1r,4r)-4-(2-fluoro-6-methylphenyl)cyclohexyl)-2-methyl-8-((3-(trifluoromethoxy)pyridin-2-yl)methyl)pyrido[2,3-d]pyrimidin-7(8H)-one (33.1 mg, 26%) as a white solid. 1H NMR (400 MHz, Chloroform-d) δ 8.78 (s, 1H), 8.27 (dd, J=4.7, 1.3 Hz, 1H), 7.65-7.53 (m, 2H), 7.21 (dd, J=8.3, 4.6 Hz, 1H), 7.04 (td, J=7.8, 5.4 Hz, 1H), 6.96-6.81 (m, 2H), 5.92 (s, 2H), 3.10 (t, J=11.7 Hz, 1H), 2.97-2.83 (m, 1H), 2.69 (s, 3H), 2.37 (s, 3H), 2.24-2.04 (m, 4H), 1.84 (d, J=12.8 Hz, 2H), 1.51 (d, J=12.7 Hz, 2H) MS m/z: 527.1 [M+H]⁺.

### 7-((1r,4r)-4-(2-Fluoro-6-methylphenyl)cyclohexyl)-5-((3-methoxypyridin-2-yl)methyl)-3-methylpyrido[2,3-b]pyrazin-6(5H)-one (199)

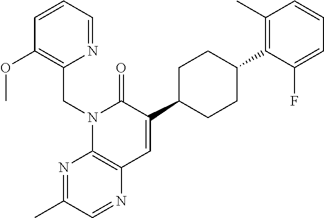 [SMILES: COc1cccnc1Cn1c(=O)c([C@H]2CC[C@H](c3c(C)cccc3F)CC2)cc2ncc(C)nc21]

Step 1: 2-(chloromethyl)-3-methoxypyridine: Followed the General Procedure J using (3-methoxypyridin-2-yl)methanol (200 mg, 1.43 mmol, 1 equiv.) and SOCl₂ (856 mg, 7.15 mmol, 5.0 equiv.) as the starting materials to give the crude product 2-(chloromethyl)-3-methoxypyridine (210 mg) as a colorless oil. MS m/z: 158 [M+H]⁺.

Step 2: 7-((1r,4r)-4-(2-fluoro-6-methylphenyl)cyclohexyl)-5-((3-methoxypyridin-2-yl)methyl)-3-methylpyrido[2,3-b]pyrazin-6(5H)-one: Followed the General Procedure E using 7-((1r,4r)-4-(2-fluoro-6-methylphenyl)cyclohexyl)-3-methylpyrido[2,3-b]pyrazin-6(5H)-one (80 mg, 0.228 mmol, 1 equiv.) and 2-(chloromethyl)-3-methoxypyridine (53.8 mg, 0.342 mmol, 1.5 equiv.) as the starting materials to give 7-((1r,4r)-4-(2-fluoro-6-methylphenyl)cyclohexyl)-5-((3-methoxypyridin-2-yl)methyl)-3-methylpyrido[2,3-b]pyrazin-6(5H)-one (28.2 mg, 24.9%) as a white solid. ¹H NMR (400 MHz, Chloroform-d) δ 8.31 (s, 1H), 7.95 (s, 1H), 7.79 (s, 1H), 7.17 (d, J=22.0 Hz, 2H), 7.06-6.98 (m, 1H), 6.91 (d, J=7.4 Hz, 1H), 6.88-6.80 (m, 1H), 5.90 (s, 2H), 3.95 (s, 3H), 3.14 (t, J=12.0 Hz, 1H), 2.90 (t, J=12.4 Hz, 1H), 2.54 (s, 3H), 2.37 (s, 3H), 2.22-2.08 (m, 4H), 1.89-1.78 (m, 2H), 1.54 (t, J=12.5 Hz, 2H). MS m/z: 473.15 [M+H]⁺.

### 7-((1S,3R)-3-(2-Fluoro-6-methylphenyl)cyclopentyl)-5-((3-(trifluoromethyl)pyridin-2-yl)methyl)pyrido[2,3-b]pyrazin-6(5H)-one (200A); 7-((1S,3S)-3-(2-Fluoro-6-methylphenyl)cyclopentyl)-5-((3-(trifluoromethyl)pyridin-2-yl)methyl)pyrido[2,3-b]pyrazin-6(5H)-one (200B); 7-((1R,3S)-3-(2-Fluoro-6-methylphenyl)cyclopentyl)-5-((3-(trifluoromethyl)pyridin-2-yl)methyl)pyrido[2,3-b]pyrazin-6(5H)-one (200C); 7-((1R,3R)-3-(2-Fluoro-6-methylphenyl)cyclopentyl)-5-((3-(trifluoromethyl)pyridin-2-yl)methyl)pyrido[2,3-b]pyrazin-6(5H)-one (200D)

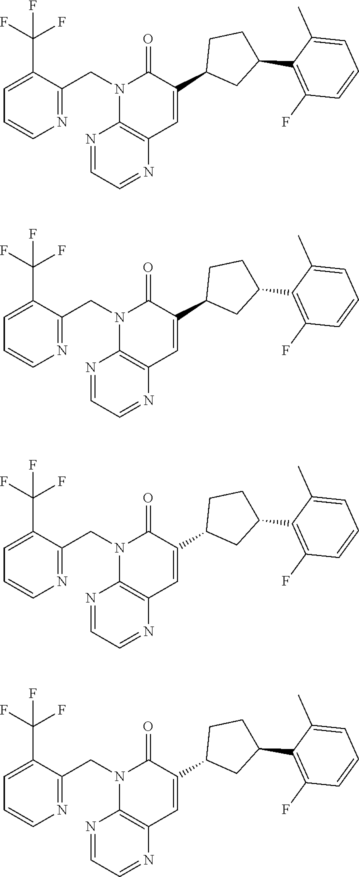 [SMILES: Cc1cccc(F)c1[C@@H]1CC[C@H](c2cc3nccnc3n(Cc3ncccc3C(F)(F)F)c2=O)C1 ; Cc1cccc(F)c1[C@H]1CC[C@H](c2cc3nccnc3n(Cc3ncccc3C(F)(F)F)c2=O)C1 ; Cc1cccc(F)c1[C@H]1CC[C@@H](c2cc3nccnc3n(Cc3ncccc3C(F)(F)F)c2=O)C1 ; Cc1cccc(F)c1[C@@H]1CC[C@@H](c2cc3nccnc3n(Cc3ncccc3C(F)(F)F)c2=O)C1]

Step 1: 7-(3-(2-fluoro-6-methylphenyl)cyclopentyl)pyrido[2,3-b]pyrazin-6(5H)-one: Followed the General Procedure D using ethyl 2-(3-(2-fluoro-6-methylphenyl)cyclopentyl)acetate (220 mg, 0.832 mmol, 1 equiv) and 3-aminopyrazine-2-carbaldehyde (153 mg, 1.24 mmol, 1.5 equiv) as the starting materials to give 7-(3-(2-fluoro-6-methylphenyl)cyclopentyl)pyrido[2,3-b]pyrazin-6(5H)-one (120 mg, 66%) as a yellow solid. MS m/z: 324 [M+H]⁺.

Step 2: 7-(3-(2-fluoro-6-methylphenyl)cyclopentyl)-5-((3-(trifluoromethyl)pyridin-2-yl)methyl)pyrido[2,3-b]pyrazin-6(5H)-one: Followed the General Procedure E using 7-(3-(2-fluoro-6-methylphenyl)cyclopentyl)pyrido[2,3-b]pyrazin-6(5H)-one (120 mg, 0.383 mmol, 1 equiv) and 2-(chloromethyl)-3-(trifluoromethyl)pyridine (89.8 mg, 0.46 mmol, 1.2 equiv) as the starting materials to give the crude product 7-(3-(2-fluoro-6-methylphenyl)cyclopentyl)-5-((3-(trifluoromethyl)pyridin-2-yl)methyl)pyrido[2,3-b]pyrazin-6(5H)-one (98 mg) as a white solid. The crude product was purified by Chiral-HPLC with the following condition (Column: CHIRALPAK IG 2*25 cm, 5 m; Mobile Phase A: Hex (10 mM NH₃-MeOH), Mobile Phase B: EtOH; Flow rate: 20 mL/min; Gradient: isocratic 20; Wave Length: 220/341 nm; RT1 (min): 7.3; RT2 (min): 9.3; Sample Solvent: EtOH; Injection Volume: 0.25 mL; Number Of Runs: 11) to afford 7-((1R,3R)-3-(2-fluoro-6-methylphenyl)cyclopentyl)-5-((3-(trifluoromethyl)pyridin-2-yl)methyl)pyrido[2,3-b]pyrazin-6(5H)-one (5.7 mg, 5.8%) as a white solid, with the following conditions (Column: Lux 5 um Cellulose-4 3*25 cm, 5 m; Mobile Phase A: Hex (10 mM NH₃-MeOH), Mobile Phase B: EtOH-HPLC; Flow rate: 40 mL/min; Gradient: isocratic 5; Wave Length: 216/260 nm; RT1 (min): 12.465; RT2 (min): 14.87; RT3 (min): 22.215; Sample Solvent: EtOH-HPLC; Injection Volume: 0.5 mL; Number Of Runs: 3) to afford 7-((1S,3R)-3-(2-fluoro-6-methylphenyl)cyclopentyl)-5-((3-(trifluoromethyl)pyridin-2-yl)methyl)pyrido[2,3-b]pyrazin-6(5H)-one (5.7 mg, 5.8%) as a white solid, 7-((1S,3S)-3-(2-fluoro-6-methylphenyl)cyclopentyl)-5-((3-(trifluoromethyl)pyridin-2-yl)methyl)pyrido[2,3-b]pyrazin-6(5H)-one (6.3 mg, 6.4%) as a white solid and 7-((1R,3S)-3-(2-fluoro-6-methylphenyl)cyclopentyl)-5-((3-(trifluoromethyl)pyridin-2-yl)methyl)pyrido[2,3-b]pyrazin-6(5H)-one (11.8 mg, 11.9%) as a white solid.

200A: 7-((1S,3R)-3-(2-fluoro-6-methylphenyl)cyclopentyl)-5-((3-(trifluoromethyl)pyridin-2-yl)methyl)pyrido[2,3-b]pyrazin-6(5H)-one. ¹H NMR (400 MHz, Chloroform-d) δ 8.47-8.38 (m, 2H), 8.34 (d, J=2.4 Hz, 1H), 7.97 (d, J=7.6 Hz, 1H), 7.92 (d, J=1.1 Hz, 1H), 7.25-7.20 (m, 1H), 7.08-6.98 (m, 1H), 6.93-6.81 (m, 2H), 6.04 (s, 2H), 3.89-3.76 (m, 1H), 3.65-3.51 (m, 1H), 2.54-2.42 (m, 1H), 2.37 (s, 4H), 2.15-2.05 (m, 2H), 1.96-1.88 (m, 1H), 1.26 (s, 1H). MS m/z: 483.05 [M+H]⁺.

200B: 7-((1S,3S)-3-(2-fluoro-6-methylphenyl)cyclopentyl)-5-((3-(trifluoromethyl)pyridin-2-yl)methyl)pyrido[2,3-b]pyrazin-6(5H)-one. ¹H NMR (400 MHz, Chloroform-d) δ 8.48-8.31 (m, 3H), 8.00-7.89 (m, 2H), 7.25-7.20 (m, 1H), 7.07-6.98 (m, 1H), 6.93-6.82 (m, 2H), 6.04 (s, 2H), 3.89-3.76 (m, 1H), 3.65-3.52 (m, 1H), 2.54-2.42 (m, 1H), 2.37 (s, 4H), 2.20-2.02 (m, 3H), 1.98-1.87 (m, 1H). MS m/z: 483.05 [M+H]⁺.

200C: 7-((1R,3S)-3-(2-fluoro-6-methylphenyl)cyclopentyl)-5-((3-(trifluoromethyl)pyridin-2-yl)methyl)pyrido[2,3-b]pyrazin-6(5H)-one. ¹H NMR (400 MHz, Chloroform-d) δ 8.48-8.39 (m, 2H), 8.34 (d, J=2.4 Hz, 1H), 8.03-7.96 (m, 2H), 7.25 (d, J=5.0 Hz, 1H), 7.09-7.00 (m, 1H), 6.95-6.83 (m, 2H), 6.04 (s, 2H), 3.57-3.45 (m, 2H), 2.37 (s, 4H), 2.34-2.25 (m, 1H), 2.18-1.98 (m, 4H). MS m/z: 483.05 [M+H]⁺.

200D: 7-((1R,3R)-3-(2-fluoro-6-methylphenyl)cyclopentyl)-5-((3-(trifluoromethyl)pyridin-2-yl)methyl)pyrido[2,3-b]pyrazin-6(5H)-one. ¹H NMR (400 MHz, Chloroform-d) δ 8.45 (d, J=2.4 Hz, 1H), 8.43-8.37 (m, 1H), 8.34 (d, J=2.4 Hz, 1H), 8.01-7.93 (m, 2H), 7.27-7.19 (m, 1H), 7.09-7.00 (m, 1H), 6.95-6.83 (m, 2H), 6.04 (s, 2H), 3.59-3.45 (m, 2H), 2.37 (s, 4H), 2.33-2.24 (m, 1H), 2.20-1.98 (m, 4H). MS m/z: 483.05 [M+H]⁺.

### 3-Ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (201)

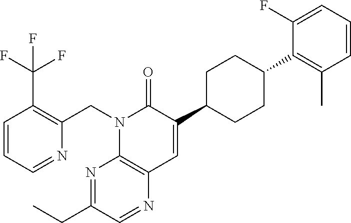 [SMILES: CCc1cnc2cc([C@H]3CC[C@H](c4c(C)cccc4F)CC3)c(=O)n(Cc3ncccc3C(F)(F)F)c2n1]

Step 1: 3-chloro-6-vinylpyrazin-2-amine: Followed the General Procedure AL using 6-bromo-3-chloropyrazin-2-amine (5 g, 24 mmol, 1 equiv) and 4,4,5,5-tetramethyl-2-vinyl-1,3,2-dioxaborolane (3.32 g, 21.5 mmol, 0.9 equiv) as the starting materials to give 3-chloro-6-vinylpyrazin-2-amine (3 g, 80%) as a yellow solid. MS m/z: 156 [M+H]⁺.

Step 2: methyl 3-amino-5-vinylpyrazine-2-carboxylate: Followed the General Procedure AI using 3-chloro-6-vinylpyrazin-2-amine (3 g, 19.3 mmol, 1 equiv) as the starting material to give methyl 3-amino-5-vinylpyrazine-2-carboxylate (2.4 g, 69%) as a colorless oil. MS m/z: 180 [M+H]⁺.

Step 3: methyl 3-amino-5-ethylpyrazine-2-carboxylate: Followed the General Procedure AJ using methyl 3-amino-5-vinylpyrazine-2-carboxylate (2.4 g, 13.4 mmol, 1 equiv) as the starting material to give the crude product methyl 3-amino-5-ethylpyrazine-2-carboxylate (2.4 g) as a colorless oil. MS m/z: 182 [M+H]⁺.

Step 4: 3-amino-5-ethylpyrazine-2-carbaldehyde: Followed the General Procedure AH using methyl 3-amino-5-ethylpyrazine-2-carboxylate (2.4 g, 13.2 mmol, 1 equiv) as the starting material to give the crude product 3-amino-5-ethylpyrazine-2-carbaldehyde (1 g) as a yellow solid. MS m/z: 152 [M+H]⁺.

Step 5: 3-ethyl-7-((1r,4r)-4-(2-fluoro-6-methylphenyl)cyclohexyl)pyrido[2,3-b]pyrazin-6(5H)-one: Followed the General Procedure D using ethyl 2-((1r,4r)-4-(2-fluoro-6-methylphenyl)cyclohexyl)acetate (300 mg, 1.08 mmol, 1 equiv) and 3-amino-5-ethylpyrazine-2-carbaldehyde (244 mg, 1.62 mmol, 1.5 equiv) as the starting materials to give 3-ethyl-7-((1r,4r)-4-(2-fluoro-6-methylphenyl)cyclohexyl)pyrido[2,3-b]pyrazin-6(5H)-one (120 mg, 30%) as a white solid. MS m/z: 366 [M+H]⁺.

Step 6: 3-ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AP using 3-ethyl-7-((1r,4r)-4-(2-fluoro-6-methylphenyl)cyclohexyl)pyrido[2,3-b]pyrazin-6(5H)-one (40 mg, 109 μmol, 1 equiv) and 2-(chloromethyl)-3-(trifluoromethyl)pyridine (25.7 mg, 131 μmol, 1.2 equiv.) as the starting materials to give 3-ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (5 mg, 9%) as a white solid. ¹H NMR (400 MHz, Chloroform-d) δ 8.47-8.41 (m, 1H), 8.30 (s, 1H), 8.00 (d, J=7.9 Hz, 1H), 7.94 (s, 1H), 7.26-7.24 (m, 1H), 7.08-6.99 (m, 1H), 6.92 (d, J=7.4 Hz, 1H), 6.85 (dd, J=11.8, 8.1 Hz, 1H), 6.06 (s, 2H), 3.17 (t, J=12.0 Hz, 1H), 2.91 (t, J=12.4 Hz, 1H), 2.81 (q, J=7.6 Hz, 2H), 2.38 (s, 3H), 2.24-2.13 (m, 4H), 1.92-1.81 (m, 2H), 1.64-1.47 (m, 2H), 1.17 (t, J=7.6 Hz, 3H). MS m/z: 525.1 [M+H]⁺.

### 3-Methyl-7-((1r,4r)-4-(2-(trifluoromethyl)phenyl)cyclohexyl)-5-((3-(trifluoromethyl)pyridin-2-yl)methyl)pyrido[2,3-b]pyrazin-6(5H)-one (202)

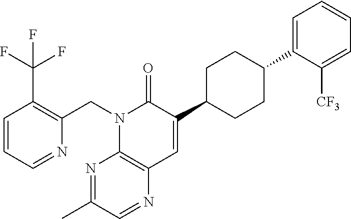 [SMILES: Cc1cnc2cc([C@H]3CC[C@H](c4ccccc4C(F)(F)F)CC3)c(=O)n(Cc3ncccc3C(F)(F)F)c2n1]

Step 1: ethyl 2-(2′-(trifluoromethyl)-2,3,4,5-tetrahydro-[1,1′-biphenyl]-4-yl)acetate: Followed the General Procedure AL using 1-bromo-2-(trifluoromethyl)benzene (490 mg, 2.17 mmol, 1 equiv.) and ethyl 2-(4-(4,4,5,5-tetramethyl-1,3,2-dioxaborolan-2-yl)cyclohex-3-en-1-yl)acetate (705 mg, 2.39 mmol, 1.1 equiv.) as the starting materials to give ethyl 2-(2′-(trifluoromethyl)-2,3,4,5-tetrahydro-[1,1′-biphenyl]-4-yl)acetate (300 mg, 44%) as a light yellow oil. MS m/z: 313 [M+H]⁺.

Step 2: ethyl 2-(4-(2-(trifluoromethyl)phenyl)cyclohexyl)acetate: Followed the General Procedure AR using ethyl 2-(2′-(trifluoromethyl)-2,3,4,5-tetrahydro-[1,1′-biphenyl]-4-yl)acetate (300 mg, 0.962 mmol, 1 equiv.) as the starting material to give the crude product ethyl 2-(4-(2-(trifluoromethyl)phenyl)cyclohexyl)acetate (240 mg) as a colorless oil. MS m/z: 315 [M+H]⁺.

Step 3: 3-methyl-7-((1r,4r)-4-(2-(trifluoromethyl)phenyl)cyclohexyl)pyrido[2,3-b]pyrazin-6(5H)-one: Followed the General General Procedure D using ethyl 2-(4-(2-(trifluoromethyl)phenyl)cyclohexyl)acetate (240 mg, 0.732 mmol, 1 equiv.) and 3-amino-5-methylpyrazine-2-carbaldehyde (158 mg, 1.15 mmol, 1.5 equiv.) as the starting materials to give 3-methyl-7-((1r,4r)-4-(2-(trifluoromethyl)phenyl)cyclohexyl)pyrido[2,3-b]pyrazin-6(5H)-one (40 mg, 13.5%) as an off-white solid. MS m/z: 388 [M+H]⁺.

Step 4: 3-methyl-7-((1r,4r)-4-(2-(trifluoromethyl)phenyl)cyclohexyl)-5-((3-(trifluoromethyl)pyridin-2-yl)methyl)pyrido[2,3-b]pyrazin-6(5H)-one: Followed the General Procedure E using 3-methyl-7-((1r,4r)-4-(2-(trifluoromethyl)phenyl)cyclohexyl)pyrido[2,3-b]pyrazin-6(5H)-one (40 mg, 0.103 mmol, 1 equiv.) and 2-(bromomethyl)-3-(trifluoromethyl)pyridine hydrobromide (37.1 mg, 0.135 mmol, 1.5 equiv.) as the starting materials to give 3-methyl-7-((1r,4r)-4-(2-(trifluoromethyl)phenyl)cyclohexyl)-5-((3-(trifluoromethyl)pyridin-2-yl)methyl)pyrido[2,3-b]pyrazin-6(5H)-one (9.2 mg, 16.4%) as a white solid. ¹H NMR (400 MHz, DMSO-d₆) δ 8.52 (d, J=4.4 Hz, 1H), 8.49 (s, 1H), 8.23 (d, J=8.0, 1.6 Hz, 1H), 7.92 (s, 1H), 7.74-7.62 (m, 3H), 7.48 (dd, J=8.0, 5.0 Hz, 1H), 7.40 (t, J=7.6 Hz, 1H), 5.88 (s, 2H), 3.07-2.85 (m, 2H), 2.47 (s, 3H), 2.01 (d, J=12.4 Hz, 2H), 1.89-1.71 (m, 4H), 1.69-1.56 (m, 2H). MS m/z: 547.10 [M+H]⁺.

### 6-((1r,4r)-4-(2-Fluoro-6-methylphenyl)cyclohexyl)-2-methyl-8-((3-(trifluoromethyl)pyridin-2-yl)methyl)pyrido[2,3-d]pyrimidin-7(8H)-one (203)

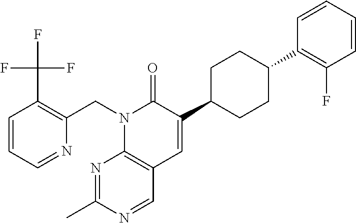 [SMILES: Cc1ncc2cc([C@H]3CC[C@H](c4ccccc4F)CC3)c(=O)n(Cc3ncccc3C(F)(F)F)c2n1]

Step 1: 6-((1r,4r)-4-(2-fluoro-6-methylphenyl)cyclohexyl)-2-methyl-8-((3-(trifluoromethyl)pyridin-2-yl)methyl)pyrido[2,3-d]pyrimidin-7(8H)-one: Followed the General Procedure E using 6-((1r,4r)-4-(2-fluoro-6-methylphenyl)cyclohexyl)-2-methylpyrido[2,3-d]pyrimidin-7(8H)-one (80 mg, 0.228 mmol, 1 equiv) and 2-(chloromethyl)-3-(trifluoromethyl)pyridine (53.4 mg, 0.274 mmol, 1.2 equiv) as the starting materials to give 6-((1r,4r)-4-(2-fluoro-6-methylphenyl)cyclohexyl)-2-methyl-8-((3-(trifluoromethyl)pyridin-2-yl)methyl)pyrido[2,3-d]pyrimidin-7(8H)-one (26.2 mg, 22.1%) as a white solid. ¹H NMR (400 MHz, Chloroform-d) δ 8.78 (s, 1H), 8.44 (d, J=4.8 Hz, 1H), 7.98 (d, J=7.9 Hz, 1H), 7.57 (s, 1H), 7.24 (d, J=5.0 Hz, 1H), 7.03 (td, J=7.8, 5.4 Hz, 1H), 6.92 (d, J=7.5 Hz, 1H), 6.84 (dd, J=11.8, 8.1 Hz, 1H), 6.00 (s, 2H), 3.10 (dd, J=13.4, 10.6 Hz, 1H), 2.97-2.84 (m, 1H), 2.67 (s, 3H), 2.37 (s, 3H), 2.14 (t, J=12.6 Hz, 4H), 1.90-1.80 (m, 2H), 1.51 (q, J=12.7 Hz, 2H). MS m/z: 511.15 [M+H]⁺.

### 3-Ethyl-7-((1r,4r)-4-(2-fluoro-6-methylphenyl)cyclohexyl)-5-((3-(trifluoromethoxy)pyridin-2-yl)methyl)pyrido[2,3-b]pyrazin-6(5H)-one (204)

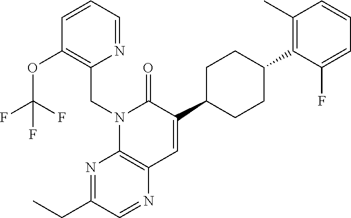 [SMILES: CCc1cnc2cc([C@H]3CC[C@H](c4c(C)cccc4F)CC3)c(=O)n(Cc3ncccc3OC(F)(F)F)c2n1]

Step 1: 3-ethyl-7-((1r,4r)-4-(2-fluoro-6-methylphenyl)cyclohexyl)-5-((3-(trifluoromethoxy)pyridin-2-yl)methyl)pyrido[2,3-b]pyrazin-6(5H)-one: Followed the General Procedure E using 3-ethyl-7-((1r,4r)-4-(2-fluoro-6-methylphenyl)cyclohexyl)pyrido[2,3-b]pyrazin-6(5H)-one (60 mg, 0.164 mmol, 1 equiv.) and 2-(chloromethyl)-3-(trifluoromethoxy)pyridine (38.2 mg, 0.180 mmol, 1.1 equiv.) as the starting materials to give 3-ethyl-7-((1r,4r)-4-(2-fluoro-6-methylphenyl)cyclohexyl)-5-((3-(trifluoromethoxy)pyridin-2-yl)methyl)pyrido[2,3-b]pyrazin-6(5H)-one (35.0 mg, 38.3%) as a white solid. ¹H NMR (400 MHz, Chloroform-d) δ 8.32 (s, 1H), 8.27 (dd, J=4.7, 1.4 Hz, 1H), 7.83 (d, J=0.9 Hz, 1H), 7.60-7.58 (m, 1H), 7.21 (dd, J=8.4, 4.7 Hz, 1H), 7.07-7.00 (m, 1H), 6.96-6.81 (m, 2H), 5.97 (s, 2H), 3.16 (t, J=12.1 Hz, 1H), 2.91 (t, J=12.3 Hz, 1H), 2.85-2.76 (m, 2H), 2.38 (s, 3H), 2.23-2.11 (m, 4H), 1.89-1.81 (m, 2H), 1.55 (t, J=12.2 Hz, 2H), 1.19 (t, J=7.6 Hz, 3H). MS m/z: 541.15[M+H]⁺.

### 3-Methyl-5-((3-(trifluoromethoxy)pyridin-2-yl)methyl)-7-((1r,4r)-4-(2-(trifluoromethyl)phenyl)cyclohexyl)pyrido[2,3-b]pyrazin-6(5H)-one (205)

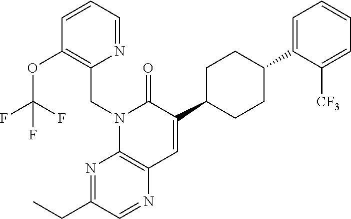 [SMILES: CCc1cnc2cc([C@H]3CC[C@H](c4ccccc4C(F)(F)F)CC3)c(=O)n(Cc3ncccc3OC(F)(F)F)c2n1]

Step 1: 3-methyl-5-((3-(trifluoromethoxy)pyridin-2-yl)methyl)-7-((1r,4r)-4-(2-(trifluoromethyl)phenyl)cyclohexyl)pyrido[2,3-b]pyrazin-6(5H)-one: Followed the General Procedure E using 5-((3-(difluoromethyl)pyridin-2-yl)methyl)-3-methyl-7-(piperidin-4-yl)pyrido[2,3-b]pyrazin-6(5H)-one (30 mg, 0.077 mmol, 1 equiv) and 2-(chloromethyl)-3-(trifluoromethoxy)pyridine (24.6 mg, 0.115 mmol, 1.5 equiv) as the starting materials to give 3-methyl-5-((3-(trifluoromethoxy)pyridin-2-yl)methyl)-7-((1r,4r)-4-(2-(trifluoromethyl)phenyl)cyclohexyl)pyrido[2,3-b]pyrazin-6(5H)-one (11.4 mg, 26.0%) as a white solid. ¹H NMR (300 MHz, Chloroform-d) δ 8.34 (s, 2H), 7.86 (s, 1H), 7.64 (s, 2H), 7.48 (s, 2H), 7.26 (s, 1H), 7.25 (s, 1H), 5.99 (s, 2H), 3.16-3.02 (m, 2H), 2.54 (s, 3H), 2.16 (d, J=11.3 Hz, 2H), 1.97 (s, 2H), 1.79-1.58 (m, 4H). MS m/z: 563.15 [M+H]⁺.

### 3-((1r,4r)-4-(2-Fluoro-6-methylphenyl)cyclohexyl)-7-methyl-1-((3-methylpyridin-2-yl)methyl)-1,8-naphthyridin-2(1H)-one (206)

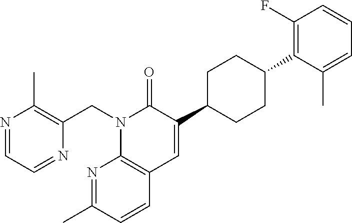 [SMILES: Cc1ccc2cc([C@H]3CC[C@H](c4c(C)cccc4F)CC3)c(=O)n(Cc3nccnc3C)c2n1]

Step 1: 3-((1r,4r)-4-(2-fluoro-6-methylphenyl)cyclohexyl)-7-methyl-1-((3-methylpyrazin-2-yl)methyl)-1,8-naphthyridin-2(1H)-one Followed the General Procedure G using 3-((1r,4r)-4-(2-fluoro-6-methylphenyl)cyclohexyl)-7-methyl-1,8-naphthyridin-2(1H)-one (50 mg, 142 μmol, 1.0 equiv) and 2-(chloromethyl)-3-methylpyrazine (20.4 mg, 142 μmol, 1.0 equiv) as the starting materials to give 3-((1r,4r)-4-(2-fluoro-6-methylphenyl)cyclohexyl)-7-methyl-1-((3-methylpyrazin-2-yl)methyl)-1,8-naphthyridin-2(1H)-one (11.1 mg, 15.3%) as a white solid. ¹H NMR (400 MHz, Chloroform-d) δ 8.25 (d, J=2.6 Hz, 1H), 8.18 (d, J=2.6 Hz, 1H), 7.74 (d, J=7.8 Hz, 1H), 7.53 (s, 1H), 7.08-6.96 (m, 2H), 6.94-6.79 (m, 2H), 5.92 (s, 2H), 3.12 (t, J=12.0 Hz, 1H), 2.90 (t, J=12.3 Hz, 1H), 2.83 (s, 3H), 2.49 (s, 3H), 2.37 (s, 3H), 2.15 (t, J=13.8 Hz, 4H), 1.84 (d, J=12.8 Hz, 2H), 1.52 (q, J=12.4 Hz, 2H). MS m/z: 457.15 [M+H]⁺.

### 7-((1r,4r)-4-(5-Fluoro-1-methyl-1H-indazol-4-yl)cyclohexyl)-3-methyl-5-((3-(trifluoromethyl)pyridin-2-yl)methyl)pyrido[2,3-b]pyrazin-6(5H)-one (207)

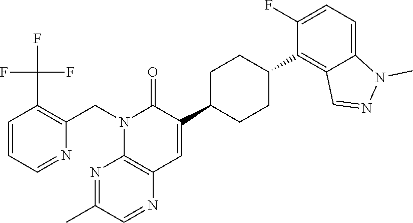 [SMILES: Cc1cnc2cc([C@H]3CC[C@H](c4c(F)ccc5c4cnn5C)CC3)c(=O)n(Cc3ncccc3C(F)(F)F)c2n1]

Step 1: 4-bromo-5-fluoro-1-methyl-1H-indazole: Followed the General Procedure AO using 2-bromo-3,6-difluorobenzaldehyde (1 g, 4.53 mmol, 1 equiv) and methylhydrazine hydrochloride (1 g, 4.98 mmol, 1.1 equiv) as the starting materials to give 4-bromo-5-fluoro-1-methyl-1H-indazole (500 mg, 48%) as a white solid. MS m/z: 229 [M+H]⁺.

Step 2: ethyl 2-(4-(5-fluoro-1-methyl-1H-indazol-4-yl)cyclohex-3-en-1-yl)acetate: Followed the General Procedure AL using 4-bromo-5-fluoro-1-methyl-1H-indazole (500 mg, 2.18 mmol, 1 equiv) and ethyl 2-(4-(4,4,5,5-tetramethyl-1,3,2-dioxaborolan-2-yl)cyclohex-3-en-1-yl)acetate (642 mg, 2.18 mmol, 1 equiv) as the starting materials to give ethyl 2-(4-(5-fluoro-1-methyl-1H-indazol-4-yl)cyclohex-3-en-1-yl)acetate (400 mg, 57%) as a white solid. MS m/z: 317 [M+H]⁺.

Step 3: ethyl 2-(4-(5-fluoro-1-methyl-1H-indazol-4-yl)cyclohexyl)acetate: Followed the General Procedure AR using ethyl 2-(4-(5-fluoro-1-methyl-1H-indazol-4-yl)cyclohex-3-en-1-yl)acetate (400 mg, 1.26 mmol, 1 equiv) as the starting material to give the crude product ethyl 2-(4-(5-fluoro-1-methyl-1H-indazol-4-yl)cyclohexyl)acetate (300 mg) as a white solid. MS m/z: 319 [M+H]⁺.

Step 4: 7-((1r,4r)-4-(5-fluoro-i-methyl-1H-indazol-4-yl)cyclohexyl)-3-methylpyrido[2,3-b]pyrazin-6(5H)-one: Followed the General Procedure D using ethyl 2-(4-(5-fluoro-i-methyl-1H-indazol-4-yl)cyclohexyl)acetate (300 mg, 0.766 mmol, 1 equiv) and 3-amino-5-methylpyrazine-2-carbaldehyde (174 mg, 0.919 mmol, 1.2 equiv) as the starting materials to give 7-((1r,4r)-4-(5-fluoro-i-methyl-1H-indazol-4-yl)cyclohexyl)-3-methylpyrido[2,3-b]pyrazin-6(5H)-one (35 mg, 9%) as a white solid. MS m/z: 392 [M+H]⁺.

Step 5: 7-((1r,4r)-4-(5-fluoro-i-methyl-1H-indazol-4-yl)cyclohexyl)-3-methyl-5-((3-(trifluoromethyl)pyridin-2-yl)methyl)pyrido[2,3-b]pyrazin-6(5H)-one: Followed the General Procedure AP using 7-((1r,4r)-4-(5-fluoro-1-methyl-1H-indazol-4-yl)cyclohexyl)-3-methylpyrido[2,3-b]pyrazin-6(5H)-one (35 mg, 0.089 mmol, 1 equiv) and 2-(chloromethyl)-3-(trifluoromethyl)pyridine hydrochloride (24.9 mg, 0.107 mmol, 1.2 equiv) as the starting materials to give 7-((1r,4r)-4-(5-fluoro-1-methyl-1H-indazol-4-yl)cyclohexyl)-3-methyl-5-((3-(trifluoromethyl)pyridin-2-yl)methyl)pyrido[2,3-b]pyrazin-6(5H)-one (5.6 mg, 10.9%) as a white solid. ¹H NMR (400 MHz, Chloroform-d) δ 8.83 (d, J=4.9 Hz, 1H), 8.66 (s, 1H), 8.16 (s, 1H), 8.12-8.03 (m, 2H), 7.47 (dd, J=8.0, 4.9 Hz, 1H), 7.20-7.07 (m, 2H), 5.98 (s, 2H), 4.05 (s, 3H), 3.30-3.11 (m, 2H), 2.80 (s, 3H), 2.38-2.07 (m, 4H), 2.04-1.96 (m, 2H), 1.74-1.64 (m, 2H). MS m/z: 551.15 [M+H]⁺.

### 1-((3-(1,1-Difluoroethyl)pyridin-2-yl)methyl)-3-((1r,4r)-4-(2-fluoro-6-methylphenyl)cyclohexyl)-7-methyl-1,8-naphthyridin-2(1H)-one (208)

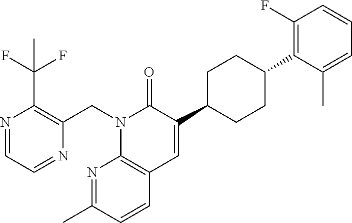 [SMILES: Cc1ccc2cc([C@H]3CC[C@H](c4c(C)cccc4F)CC3)c(=O)n(Cc3nccnc3C(C)(F)F)c2n1]

Step 1: 1-((3-(1,1-difluoroethyl)pyridin-2-yl)methyl)-3-((1r,4r)-4-(2-fluoro-6-methylphenyl)cyclohexyl)-7-methyl-1,8-naphthyridin-2(1H)-one: Followed the General Procedure AP using 3-((1r,4r)-4-(2-fluoro-6-methylphenyl)cyclohexyl)-7-methyl-1,8-naphthyridin-2(1H)-one (50 mg, 142 μmol, 1.0 equiv) and 2-(chloromethyl)-3-(1,1-difluoroethyl)pyridine (27.2 mg, 142 μmol, 1.0 equiv) as the starting materials to give 1-((3-(1,1-difluoroethyl)pyridin-2-yl)methyl)-3-((1r,4r)-4-(2-fluoro-6-methylphenyl)cyclohexyl)-7-methyl-1,8-naphthyridin-2(1H)-one (11.1 mg, 15.3%) as a white solid. ¹H NMR (400 MHz, Chloroform-d) δ 8.48 (s, 1H), 7.95 (d, J=7.8 Hz, 1H), 7.76 (d, J=7.8 Hz, 1H), 7.55 (s, 1H), 7.31-7.28 (m, 1H), 7.05-6.97 (m, 2H), 6.93-6.80 (m, 2H), 6.07 (s, 2H), 3.11 (t, J=12.0 Hz, 1H), 2.89 (s, 1H), 2.48 (s, 3H), 2.37 (s, 3H), 2.28 (t, J=18.4 Hz, 3H), 2.15 (t, J=11.2 Hz, 4H), 1.83 (d, J=13.2 Hz, 2H), 1.52 (d, J=12.4 Hz, 2H). MS m/z: 506.15 [M+H]⁺.

### 7-((1r,4r)-4-(2,6-Difluorophenyl)cyclohexyl)-3-methyl-5-((3-(trifluoromethoxy)pyridin-2-yl)methyl)pyrido[2,3-b]pyrazin-6(5H)-one (209)

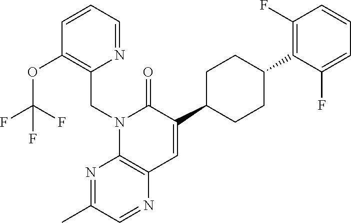 [SMILES: Cc1cnc2cc([C@H]3CC[C@H](c4c(F)cccc4F)CC3)c(=O)n(Cc3ncccc3OC(F)(F)F)c2n1]

Step 1: 7-((1r,4r)-4-(2,6-difluorophenyl)cyclohexyl)-3-methyl-5-((3-(trifluoromethoxy)pyridin-2-yl)methyl)pyrido[2,3-b]pyrazin-6(5H)-one: Followed the General Procedure AP using 7-((1r,4r)-4-(2,6-difluorophenyl)cyclohexyl)-3-methylpyrido[2,3-b]pyrazin-6(5H)-one (100 mg, 0.281 mmol, 1 equiv) and 2-(chloromethyl)-3-(trifluoromethoxy)pyridine (65.5 mg, 0.309 mmol, 1.1 equiv) as the starting materials to give 7-((1r,4r)-4-(2,6-difluorophenyl)cyclohexyl)-3-methyl-5-((3-(trifluoromethoxy)pyridin-2-yl)methyl)pyrido[2,3-b]pyrazin-6(5H)-one (21.3 mg, 14.2%) as a white solid. ¹H NMR (400 MHz, Chloroform-d) δ 8.37-8.24 (m, 2H), 7.79 (s, 1H), 7.59 (d, J=8.4 Hz, 1H), 7.24-7.16 (m, 1H), 7.16-7.05 (m, 1H), 6.82 (t, J=8.6 Hz, 2H), 5.95 (s, 2H), 3.19-3.05 (m, 2H), 2.53 (s, 3H), 2.13 (t, J=12.3 Hz, 4H), 1.94-1.86 (m, 2H), 1.60-1.47 (m, 2H). MS m/z: 531.15 [M+H]⁺.

### 1-((3-(Difluoromethyl)pyrazin-2-yl)methyl)-3-((1r,4r)-4-(2-fluoro-6-methylphenyl)cyclohexyl)-7-methyl-1,8-naphthyridin-2(1H)-one (210)

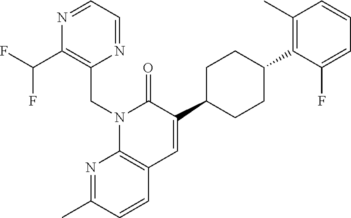 [SMILES: Cc1ccc2cc([C@H]3CC[C@H](c4c(C)cccc4F)CC3)c(=O)n(Cc3nccnc3C(F)F)c2n1]

Step 1: 1-((3-(difluoromethyl)pyrazin-2-yl)methyl)-3-((1r,4r)-4-(2-fluoro-6-methylphenyl)cyclohexyl)-7-methyl-1,8-naphthyridin-2(1H)-one: Followed the General Procedure AP using 3-((1r,4r)-4-(2-fluoro-6-methylphenyl)cyclohexyl)-7-methyl-1,8-naphthyridin-2(1H)-one (20 mg, 0.057 mmol, 1 equiv) and 2-(chloromethyl)-3-(difluoromethyl)pyrazine (15.3 mg, 0.086 mmol, 1.5 equiv) as the starting materials to give 1-((3-(difluoromethyl)pyrazin-2-yl)methyl)-3-((1r,4r)-4-(2-fluoro-6-methylphenyl)cyclohexyl)-7-methyl-1,8-naphthyridin-2(1H)-one (7.5 mg, 25.7%) as a white solid. ¹H NMR (400 MHz, Chloroform-d) δ 8.42 (s, 2H), 7.75 (d, J=7.7 Hz, 1H), 7.54 (s, 1H), 7.20 (s, 1H), 7.05-6.98 (m, 2H), 6.92 (d, J=7.6 Hz, 1H), 6.85 (dd, J=11.8, 8.1 Hz, 1H), 6.14 (s, 2H), 3.12 (t, J=12.2 Hz, 1H), 2.90 (s, 1H), 2.49 (s, 3H), 2.37 (s, 3H), 2.15 (t, J=13.8 Hz, 4H), 1.84 (d, J=12.6 Hz, 2H), 1.49 (dd, J=23.9, 11.8 Hz, 2H). MS m/z: 493.15 [M+H]⁺.

### 2-Ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (211)

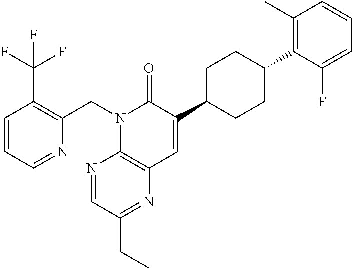 [SMILES: CCc1cnc2c(cc([C@H]3CC[C@H](c4c(C)cccc4F)CC3)c(=O)n2Cc2ncccc2C(F)(F)F)n1]

Step 1: methyl 3-amino-6-vinylpyrazine-2-carboxylate: Followed the General Procedure AL using methyl 3-amino-6-bromopyrazine-2-carboxylate (1 g, 4.310 mmol, 1 equiv) and ethenylboronic acid (0.37 g, 5.17 mmol, 1.2 equiv) as the starting materials to give methyl 3-amino-6-ethenylpyrazine-2-carboxylate (500 mg, 61.%) as a yellow solid. MS m/z: 180 [M+H]⁺.

Step 2: methyl 3-amino-6-ethylpyrazine-2-carboxylate: Followed the General Procedure AJ using methyl 3-amino-6-ethenylpyrazine-2-carboxylate (500 mg, 2.79 mmol, 1 equiv) as the starting material to give the crude product methyl 3-amino-6-ethylpyrazine-2-carboxylate (350 mg) as a yellow solid. MS m/z: 182 [M+H]⁺.

Step 3: 3-amino-6-ethylpyrazine-2-carbaldehyde: Followed the General Procedure AH using methyl 3-amino-6-ethylpyrazine-2-carboxylate (290 mg, 1.60 mmol, 1 equiv) as the starting material to give 3-amino-6-ethylpyrazine-2-carbaldehyde (180 mg, 69%) as a colorless oil. MS m/z: 152 [M+H]⁺.

Step 4: 2-ethyl-7-((1r,4r)-4-(2-fluoro-6-methylphenyl)cyclohexyl)pyrido[2,3-b]pyrazin-6(5H)-one: Followed the General Procedure D using 3-amino-6-ethylpyrazine-2-carbaldehyde (170 mg, 1.11 mmol, 1 equiv) and ethyl 2-((1r,4r)-4-(2-fluoro-6-methylphenyl)cyclohexyl)acetate (310 mg, 1.11 mmol, 1 equiv) as the starting materials to give 2-ethyl-7-((1r,4r)-4-(2-fluoro-6-methylphenyl)cyclohexyl)pyrido[2,3-b]pyrazin-6(5H)-one (75 mg, 18%) as a white solid. MS m/z: 366 [M+H]⁺.

Step 5: 2-ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AP using 2-ethyl-7-((1r,4r)-4-(2-fluoro-6-methylphenyl)cyclohexyl)pyrido[2,3-b]pyrazin-6(5H)-one (70 mg, 0.192 mmol, 1 equiv) and 2-(chloromethyl)-3-(trifluoromethyl)pyridine (44.9 mg, 0.230 mmol, 1.2 equiv) as the starting materials to give 2-ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (30 mg, 28.8%) as a white solid. ¹H NMR (400 MHz, Chloroform-d) δ 8.42 (dd, J=5.1, 1.6 Hz, 1H), 8.25 (s, 1H), 7.96 (dd, J=7.9, 1.7 Hz, 1H), 7.84 (d, J=0.9 Hz, 1H), 7.23 (dd, J=8.0, 4.9 Hz, 1H), 7.07-6.99 (m, 1H), 6.91 (d, J=7.4 Hz, 1H), 6.89-6.80 (m, 1H), 6.03 (s, 2H), 3.20-3.11 (m, 1H), 2.96-2.85 (m, 3H), 2.37 (s, 3H), 2.23-2.10 (m, 4H), 1.90-1.79 (m, 2H), 1.60-1.47 (m, 2H), 1.38 (t, J=7.6 Hz, 3H). MS m/z: 525.15 [M+H]⁺.

### 5-((3-(Difluoromethoxy)pyridin-2-yl)methyl)-7-((1r,4r)-4-(2-fluoro-6-methylphenyl)cyclohexyl)-3-methylpyrido[2,3-b]pyrazin-6(5H)-one (212)

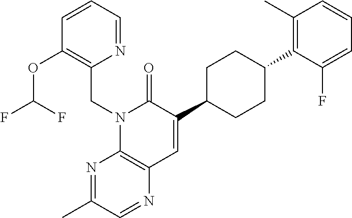 [SMILES: Cc1cnc2cc([C@H]3CC[C@H](c4c(C)cccc4F)CC3)c(=O)n(Cc3ncccc3OC(F)F)c2n1]

Step 1: 5-((3-(difluoromethoxy)pyridin-2-yl)methyl)-7-((1r,4r)-4-(2-fluoro-6-methylphenyl)cyclohexyl)-3-methylpyrido[2,3-b]pyrazin-6(5H)-one: Followed the General Procedure AP using 7-((1r,4r)-4-(2-fluoro-6-methylphenyl)cyclohexyl)-3-methylpyrido[2,3-b]pyrazin-6(5H)-one (50 mg, 0.151 mmol, 1 equiv.) and 2-(chloromethyl)-3-(difluoromethoxy)pyridine (35 mg, 0.181 mmol, 1.2 equiv.) as the starting materials to give 5-((3-(difluoromethoxy)pyridin-2-yl)methyl)-7-((1r,4r)-4-(2-fluoro-6-methylphenyl)cyclohexyl)-3-methylpyrido[2,3-b]pyrazin-6(5H)-one (17.4 mg, 22.4%) as a white solid. ¹H NMR (400 MHz, Chloroform-d) δ 8.33 (s, 1H), 8.24 (d, J=4.8 Hz, 1H), 7.80 (s, 1H), 7.54 (d, J=8.0 Hz, 1H), 7.20 (dd, J=8.4, 4.7 Hz, 1H), 7.08-6.99 (m, 1H), 6.95-6.52 (m, 3H), 5.94 (s, 2H), 3.12 (t, J=12.0 Hz, 1H), 2.88 (t, J=12.0 Hz, 1H), 2.56 (s, 3H), 2.37 (s, 3H), 2.19-2.10 (m, 4H), 1.89-1.80 (m, 2H), 1.58-1.46 (m, 2H). MS m/z: 509.15 [M+H]⁺.

### 7-((1r,4r)-4-(2-Fluoro-6-methylphenyl)cyclohexyl)-3-methyl-5-((3-(2,2,2-trifluoroethoxy)pyridin-2-yl)methyl)pyrido[2,3-b]pyrazin-6(5H)-one (213)

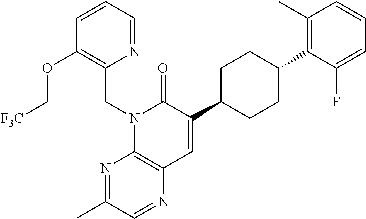 [SMILES: Cc1cnc2cc([C@H]3CC[C@H](c4c(C)cccc4F)CC3)c(=O)n(Cc3ncccc3OCC(F)(F)F)c2n1]

Step 1: 3-(2,2,2-trifluoroethoxy)pyridine-2-carboxylate: Followed the General Procedure AK using methyl 3-chloropyridine-2-carboxylate (1 g, 5.828 mmol, 1 equiv) as the starting material, trifluoroethanol (10 mL) as the solvent to give methyl 3-(2,2,2-trifluoroethoxy)pyridine-2-carboxylate (300 mg, 21%) as a yellow solid. MS m/z: 236 [M+H]⁺.

Step 2: [3-(2,2,2-trifluoroethoxy)pyridin-2-yl]methanol: Followed the General Procedure I using methyl 3-(2,2,2-trifluoroethoxy)pyridine-2-carboxylate (300 mg, 1.276 mmol, 1 equiv) as the starting material to give (3-(2,2,2-trifluoroethoxy)pyridin-2-yl)methanol (100 mg, 37%) as a colorless oil. MS m/z: 208 [M+H]⁺.

Step 3: 2-(chloromethyl)-3-(2,2,2-trifluoroethoxy)pyridine: Followed the General Procedure J using (3-(2,2,2-trifluoroethoxy)pyridin-2-yl)methanol (155 mg, 0.748 mmol, 1 equiv) as the starting material to give the crude product 2-(chloromethyl)-3-(2,2,2-trifluoroethoxy)pyridine (100 mg) as a yellow solid. MS m/z: 226 [M+H]⁺.

Step 4: 7-((1r,4r)-4-(2-fluoro-6-methylphenyl)cyclohexyl)-3-methyl-5-((3-(2,2,2-trifluoroethoxy)pyridin-2-yl)methyl)pyrido[2,3-b]pyrazin-6(5H)-one: Followed the General Procedure AP using 2-(chloromethyl)-3-(2,2,2-trifluoroethoxy)pyridine (55 mg, 0.244 mmol, 1 equiv) and 7-((1r,4r)-4-(2-fluoro-6-methylphenyl)cyclohexyl)-3-methylpyrido[2,3-b]pyrazin-6(5H)-one (102.81 mg, 0.293 mmol, 1.2 equiv) as the starting materials to give 7-((1r,4r)-4-(2-fluoro-6-methylphenyl)cyclohexyl)-3-methyl-5-((3-(2,2,2-trifluoroethoxy)pyridin-2-yl)methyl)pyrido[2,3-b]pyrazin-6(5H)-one (17.2 mg, 12.4%) as a white solid. ¹H NMR (400 MHz, Chloroform-d) δ 8.32 (s, 1H), 8.05 (dd, J=4.6, 1.4 Hz, 1H), 7.79 (s, 1H), 7.21-7.09 (m, 2H), 7.03 (td, J=7.8, 5.4 Hz, 1H), 6.91 (d, J=7.5 Hz, 1H), 6.84 (dd, J=11.7, 8.2 Hz, 1H), 5.92 (s, 2H), 4.49 (q, J=8.0 Hz, 2H), 3.13 (ddd, J=12.2, 9.4, 3.1 Hz, 1H), 2.96-2.83 (m, 1H), 2.54 (s, 3H), 2.37 (s, 3H), 2.24-2.08 (m, 4H), 1.84 (dd, J=13.4, 3.9 Hz, 2H), 1.60-1.44 (m, 2H). MS m/z: 541.15 [M+H]⁺.

### 7-((1r,4r)-4-(2-Fluoro-6-methylphenyl)cyclohexyl)-5-((3-methoxypyridin-2-yl)methyl)-2-(trifluoromethyl)pyrido[2,3-b]pyrazin-6(5H)-one (214)

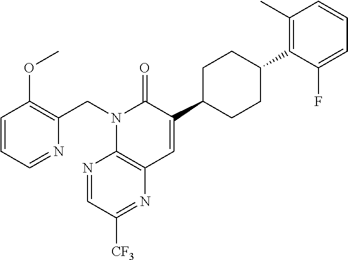 [SMILES: COc1cccnc1Cn1c(=O)c([C@H]2CC[C@H](c3c(C)cccc3F)CC2)cc2nc(C(F)(F)F)cnc21]

Step 1: 3-bromo-5-(trifluoromethyl)pyrazin-2-amine: To a stirred solution of 5-(trifluoromethyl)pyrazin-2-amine (1 g, 6.13 mmol, 1 equiv) in AcOH (10 mL) were added NBS (3.27 g, 18.3 mmol, 3 equiv) at room temperature. The resulting mixture was stirred for overnight at 60° C. The residue was purified by silica gel column chromatography, eluted with PE/EA (5:1) to afford 3-bromo-5-(trifluoromethyl)pyrazin-2-amine (450 mg, 27%) as a yellow solid. MS m/z: 242 [M+H]⁺.

Step 2: methyl 3-amino-6-(trifluoromethyl)pyrazine-2-carboxylate: Followed the General Procedure AI using 3-bromo-5-(trifluoromethyl)pyrazin-2-amine (450 mg, 1.86 mmol, 1 equiv) as the starting material to give methyl 3-amino-6-(trifluoromethyl)pyrazine-2-carboxylate (250 mg, 57%) as a white solid. MS m/z: 222 [M+H]⁺.

Step 3: 3-amino-6-(trifluoromethyl)pyrazin-2-methanol: Followed the General Procedure AH using methyl 3-amino-6-(trifluoromethyl)pyrazine-2-carboxylate (240 mg, 1.085 mmol, 1 equiv) and as the starting material to give 3-amino-6-(trifluoromethyl)pyrazin-2-methanol (200 mg, 89%) as a white oil. MS m/z: 192 [M+H]⁺.

Step 4: 7-((1r,4r)-4-(2-fluoro-6-methylphenyl)cyclohexyl)-2-(trifluoromethyl)pyrido[2,3-b]pyrazin-6(5H)-one: Followed the General Procedure D using 3-amino-6-(trifluoromethyl)pyrazin-2-methanol (80 mg, 0.419 mmol, 1 equiv) and ethyl 2-((1r,4r)-4-(2-fluoro-6-methylphenyl)cyclohexyl)acetate (139 mg, 0.503 mmol, 1.2 equiv) as the starting materials to give 7-((1r,4r)-4-(2-fluoro-6-methylphenyl)cyclohexyl)-2-(trifluoromethyl)pyrido[2,3-b]pyrazin-6(5H)-one (25 mg, 41%) as a light brown solid. MS m/z: 366 [M+H]⁺.

Step 5: 2-ethyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure E using 7-((1r,4r)-4-(2-fluoro-6-methylphenyl)cyclohexyl)-2-(trifluoromethyl)pyrido[2,3-b]pyrazin-6(5H)-one (20 mg, 0.049 mmol, 1 equiv) and 2-(chloromethyl)-3-methoxypyridine (9.33 mg, 0.059 mmol, 1.2 equiv) as the starting materials to give 7-((1r,4r)-4-(2-fluoro-6-methylphenyl)cyclohexyl)-5-((3-methoxypyridin-2-yl)methyl)-2-(trifluoromethyl)pyrido[2,3-b]pyrazin-6(5H)-one (8.7 mg, 31%) as a white solid. ¹H NMR (300 MHz, Chloroform-d) δ 8.69 (s, 1H), 7.87 (d, J=7.9 Hz, 2H), 7.21-6.99 (m, 3H), 6.96-6.82 (m, 2H), 5.86 (s, 2H), 3.95 (s, 3H), 3.18 (t, J=11.9 Hz, 1H), 2.89 (t, J=12.3 Hz, 1H), 2.38 (s, 3H), 2.14 (t, J=12.3 Hz, 4H), 1.86 (d, J=11.0 Hz, 2H), 1.55 (t, J=12.4 Hz, 2H). MS m/z: 527.15 [M+H]⁺.

### 2-Ethyl-7-((1r,4r)-4-(2-fluoro-6-methylphenyl)cyclohexyl)-5-((3-(trifluoromethoxy)pyridin-2-yl)methyl)pyrido[2,3-b]pyrazin-6(5H)-one (215)

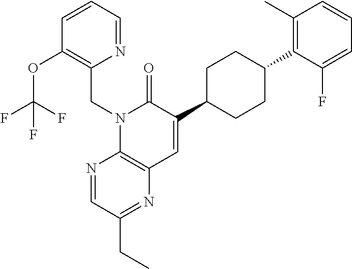 [SMILES: CCc1cnc2c(cc([C@H]3CC[C@H](c4c(C)cccc4F)CC3)c(=O)n2Cc2ncccc2OC(F)(F)F)n1]

Step 1: 2-ethyl-7-((1r,4r)-4-(2-fluoro-6-methylphenyl)cyclohexyl)-5-((3-(trifluoromethoxy)pyridin-2-yl)methyl)pyrido[2,3-b]pyrazin-6(5H)-one: Followed the General Procedure AP using 2-ethyl-7-((1r,4r)-4-(2-fluoro-6-methylphenyl)cyclohexyl)pyrido[2,3-b]pyrazin-6(5H)-one (40 mg, 0.109 mmol, 1 equiv.) and 2-(chloromethyl)-3-(trifluoromethoxy)pyridine (25.4 mg, 0.120 mmol, 1.1 equiv.) as the starting materials to give 2-ethyl-7-((1r,4r)-4-(2-fluoro-6-methylphenyl)cyclohexyl)-5-((3-(trifluoromethoxy)pyridin-2-yl)methyl)pyrido[2,3-b]pyrazin-6(5H)-one (16.1 mg, 26.6%) as an off-white solid. ¹H NMR (400 MHz, Chloroform-d) δ 8.31-8.25 (m, 2H), 7.87 (s, 1H), 7.60 (d, J=8.4, 1.6 Hz, 1H), 7.22 (dd, J=8.4, 4.8 Hz, 1H), 7.07-6.99 (m, 1H), 6.92 (d, J=7.6 Hz, 1H), 6.88-6.81 (m, 1H), 5.94 (s, 2H), 3.16 (t, J=12.0 Hz, 1H), 2.98-2.85 (m, 3H), 2.37 (s, 3H), 2.19-2.10 (m, 4H), 1.87-1.82 (m, 2H), 1.58-1.47 (m, 2H), 1.38 (t, J=7.6 Hz, 3H). MS m/z: 541.20 [M+H]⁺.

### 7-((1r,4r)-4-(2-Fluoro-6-(trifluoromethyl)phenyl)cyclohexyl)-3-methyl-5-((3-(trifluoromethoxy)pyridin-2-yl)methyl)pyrido[2,3-b]pyrazin-6(5H)-one (216)

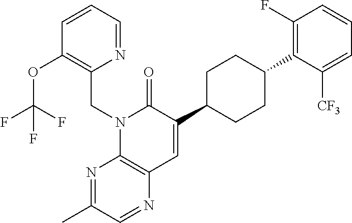 [SMILES: Cc1cnc2cc([C@H]3CC[C@H](c4c(F)cccc4C(F)(F)F)CC3)c(=O)n(Cc3ncccc3OC(F)(F)F)c2n1]

Step 1: ethyl 2-(2′-fluoro-6′-(trifluoromethyl)-2,3,4,5-tetrahydro-[1,1′-biphenyl]-4-yl)acetate: Followed the General Procedure AL using 2-bromo-1-fluoro-3-(trifluoromethyl)benzene (500 mg, 2.058 mmol, 1 equiv) and ethyl 2-(4-(4,4,5,5-tetramethyl-1,3,2-dioxaborolan-2-yl)cyclohex-3-en-1-yl)acetate (726.42 mg, 2.470 mmol, 1.2 equiv) as the starting materials to give ethyl 2-(2′-fluoro-6′-(trifluoromethyl)-2,3,4,5-tetrahydro-[1,1′-biphenyl]-4-yl)acetate (500 mg, 73%) as a yellow oil. MS m/z: 331 [M+H]⁺.

Step 2: 7-(2′-fluoro-6′-(trifluoromethyl)-2,3,4,5-tetrahydro-[1,1′-biphenyl]-4-yl)-3-methylpyrido[2,3-b]pyrazin-6(5H)-one: Followed the General Procedure D using ethyl 2-(2′-fluoro-6′-(trifluoromethyl)-2,3,4,5-tetrahydro-[1,1′-biphenyl]-4-yl)acetate (520 mg, 1.57 mmol, 1 equiv) and 3-amino-5-methylpyrazine-2-carbaldehyde (236 mg, 1.73 mmol, 1.1 equiv) as the starting materials to give 7-(2′-fluoro-6′-(trifluoromethyl)-2,3,4,5-tetrahydro-[1,1′-biphenyl]-4-yl)-3-methylpyrido[2,3-b]pyrazin-6(5H)-one (400 mg, 63%). MS m/z: 404 [M+H]⁺.

Step 3: 7-((1r,4r)-4-(2-fluoro-6-(trifluoromethyl)phenyl)cyclohexyl)-3-methylpyrido[2,3-b]pyrazin-6(5H)-one: Followed the General Procedure AR using 7-(2′-fluoro-6′-(trifluoromethyl)-2,3,4,5-tetrahydro-[1,1′-biphenyl]-4-yl)-3-methylpyrido[2,3-b]pyrazin-6(5H)-one (390 mg, 0.967 mmol, 1 equiv) as the starting material to give 7-((1r,4r)-4-(2-fluoro-6-(trifluoromethyl)phenyl)cyclohexyl)-3-methylpyrido[2,3-b]pyrazin-6(5H)-one (300 mg, 77%) as a yellow solid. MS m/z: 406 [M+H]⁺.

Step 4: 7-((1r,4r)-4-(2-fluoro-6-(trifluoromethyl)phenyl)cyclohexyl)-3-methyl-5-((3-(trifluoromethoxy)pyridin-2-yl)methyl)pyrido[2,3-b]pyrazin-6(5H)-one: Followed the General Procedure E using 7-((1r,4r)-4-(2-fluoro-6-(trifluoromethyl)phenyl)cyclohexyl)-3-methylpyrido[2,3-b]pyrazin-6(5H)-one (60 mg, 0.148 mmol, 1 equiv) and 2-(chloromethyl)-3-(trifluoromethoxy)pyridine hydrochloride (54.8 mg, 0.222 mmol, 3.0 equiv) as the starting materials to give 7-((1r,4r)-4-(2-fluoro-6-(trifluoromethyl)phenyl)cyclohexyl)-3-methyl-5-((3-(trifluoromethoxy)pyridin-2-yl)methyl)pyrido[2,3-b]pyrazin-6(5H)-one (37.1 mg, 43.1%) as a white solid. ¹H NMR (400 MHz, Chloroform-d) δ 8.29 (d, J=4.6 Hz, 1H), 7.96 (s, 1H), 7.55 (d, J=8.2 Hz, 1H), 7.43 (d, J=7.9 Hz, 1H), 7.31 (td, J=8.0, 5.3 Hz, 1H), 7.19 (td, J=8.3, 4.3 Hz, 2H), 5.67-5.52 (m, 2H), 5.52-5.41 (m, 1H), 3.38-3.14 (m, 2H), 2.84 (q, J=6.8, 6.4 Hz, 1H), 2.47-2.09 (m, 8H), 2.01 (d, J=12.5 Hz, 1H), 1.76-1.42 (m, 1H). MS m/z: 581.15 [M+H]⁺.

### 3-Methyl-7-[(1r,4r)-4-(6-fluoro-1-methylindazol-7-yl)cyclohexyl]-5-{[3-(trifluoromethyl)pyridin-2-yl]methyl}pyrido[2,3-b]pyrazin-6-one (217)

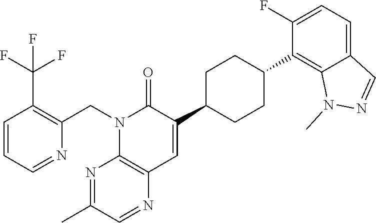 [SMILES: Cc1cnc2cc([C@H]3CC[C@H](c4c(F)ccc5cnn(C)c54)CC3)c(=O)n(Cc3ncccc3C(F)(F)F)c2n1]

Step 1: 7-bromo-6-fluoro-1-methylindazole: Followed the General Procedure AO using 3-bromo-2,4-difluorobenzaldehyde (500 mg, 2.26 mmol, 1 equiv) and methyl hydrazine hydrochloride (114 mg, 2.48 mmol, 1.1 equiv) as the starting materials to give 7-bromo-6-fluoro-1-methylindazole (260 mg, 50%) as a yellow solid. MS m/z: 229 [M+H]⁺.

Step 2: ethyl 2-[4-(6-fluoro-1-methylindazol-7-yl)cyclohex-3-en-1-yl]acetate: Followed the General Procedure AL using 7-bromo-6-fluoro-1-methylindazole (500 mg, 2.18 mmol, 1 equiv) and ethyl 2-(4-(4,4,5,5-tetramethyl-1,3,2-dioxaborolan-2-yl)cyclohex-3-en-1-yl)acetate (642 mg, 2.18 mmol, 1 equiv) as the starting materials to give ethyl 2-(4-(6-fluoro-1-methyl-1H-indazol-7-yl)cyclohex-3-en-1-yl)acetate (450 mg, 65.%) as a yellow solid. MS m/z: 317 [M+H]⁺.

Step 3: 7-(4-(6-fluoro-1-methyl-1H-indazol-7-yl)cyclohex-3-en-1-yl)-3-methylpyrido[2,3-b]pyrazin-6(5H)-one: Followed the General Procedure D using ethyl 2-(4-(6-fluoro-i-methyl-1H-indazol-7-yl)cyclohex-3-en-1-yl)acetate (500 mg, 1.58 mmol, 1 equiv) and 3-amino-5-methylpyrazine-2-carbaldehyde (260 mg, 1.89 mmol, 1.2 equiv) as the starting materials to give 7-(4-(6-fluoro-i-methyl-1H-indazol-7-yl)cyclohex-3-en-1-yl)-3-methylpyrido[2,3-b]pyrazin-6(5H)-one (160 mg, 26%) as a yellow solid. MS m/z: 390 [M+H]⁺.

Step 4: 7-((1r,4r)-4-(6-fluoro-i-methyl-1H-indazol-7-yl)cyclohexyl)-3-methylpyrido[2,3-b]pyrazin-6(5H)-one: Followed the General Procedure AR using 7-(4-(6-fluoro-i-methyl-1H-indazol-7-yl)cyclohex-3-en-1-yl)-3-methylpyrido[2,3-b]pyrazin-6(5H)-one (160 mg, 0.026 mmol, 1 equiv) as the starting material to give the crude product 7-((1r,4r)-4-(6-fluoro-1-methyl-1H-indazol-7-yl)cyclohexyl)-3-methylpyrido[2,3-b]pyrazin-6(5H)-one (40 mg) as a yellow solid. MS m/z: 392 [M+H]⁺.

Step 5: 7-((1r,4r)-4-(6-fluoro-1-methyl-1H-indazol-7-yl)cyclohexyl)-3-methyl-5-((3-(trifluoromethyl)pyridin-2-yl)methyl)pyrido[2,3-b]pyrazin-6(5H)-one: Followed the General Procedure AP using 7-((1r,4r)-4-(6-fluoro-1-methyl-1H-indazol-7-yl)cyclohexyl)-3-methylpyrido[2,3-b]pyrazin-6(5H)-one (40 mg, 0.102 mmol, 1 equiv) and 2-(chloromethyl)-3-(trifluoromethyl)pyridine (25.9 mg, 0.133 mmol, 1.3 equiv) as the starting materials to give 7-((1r,4r)-4-(6-fluoro-1-methyl-1H-indazol-7-yl)cyclohexyl)-3-methyl-5-((3-(trifluoromethyl)pyridin-2-yl)methyl)pyrido[2,3-b]pyrazin-6(5H)-one (16.6 mg, 29.5%) as a white solid. ¹H NMR (400 MHz, DMSO-d₆) δ 8.57 (s, 1H), 8.20 (d, J=7.7 Hz, 1H), 8.05 (d, J=5.7 Hz, 2H), 7.68 (s, 1H), 7.48 (s, 1H), 7.08-6.91 (m, 1H), 5.79 (s, 1H), 5.45 (d, J=25.2 Hz, 2H), 3.98 (d, J=16.7 Hz, 3H), 3.18 (s, 2H), 2.96-2.77 (m, 1H), 2.29 (d, J=26.5 Hz, 7H), 1.97 (s, 2H), 1.78-1.49 (m, 1H). MS m/z: 551.15 [M+H]⁺.

### 7-((1r,4r)-4-(1,5-Dimethyl-1H-indazol-4-yl)cyclohexyl)-3-methyl-5-((3-(trifluoromethyl)pyridin-2-yl)methyl)pyrido[2,3-b]pyrazin-6(5H)-one (218)

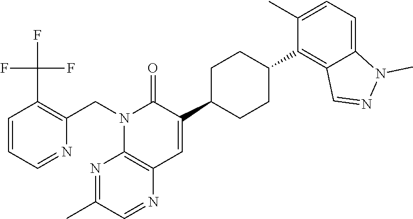 [SMILES: Cc1cnc2cc([C@H]3CC[C@H](c4c(C)ccc5c4cnn5C)CC3)c(=O)n(Cc3ncccc3C(F)(F)F)c2n1]

Step 1: ethyl 2-(4-(1,5-dimethyl-1H-indazol-4-yl)cyclohex-3-en-1-yl)acetate: Followed the General Procedure AL using 4-bromo-1,5-dimethyl-1H-indazole (500 mg, 2.22 mmol, 1 equiv) and ethyl 2-(4-(4,4,5,5-tetramethyl-1,3,2-dioxaborolan-2-yl)cyclohex-3-en-1-yl)acetate (654 mg, 2.22 mmol, 1.0 equiv.) as the starting materials to give ethyl 2-(4-(1,5-dimethyl-1H-indazol-4-yl)cyclohex-3-en-1-yl)acetate (590 mg, 85%) as a light brown oil. MS m/z: 313[M+H]⁺.

Step 2: 7-(4-(1,5-dimethyl-1H-indazol-4-yl)cyclohex-3-en-1-yl)-3-methylpyrido[2,3-b]pyrazin-6(5H)-one: Followed the General Procedure D using ethyl 2-(4-(1,5-dimethyl-1H-indazol-4-yl)cyclohex-3-en-1-yl)acetate (590 mg, 1.30 mmol, 1 equiv.) and 3-amino-5-methylpyrazine-2-carbaldehyde (309 mg, 1.56 mmol, 1.2 equiv.) as the starting materials to give 7-(4-(1,5-dimethyl-1H-indazol-4-yl)cyclohex-3-en-1-yl)-3-methylpyrido[2,3-b]pyrazin-6(5H)-one (190 mg, 26.1%) as an off-white oil. MS m/z: 386 [M+H]⁺.

Step 3: 7-((1r,4r)-4-(1,5-dimethyl-1H-indazol-4-yl)cyclohexyl)-3-methylpyrido[2,3-b]pyrazin-6(5H)-one: Followed the General Procedure AR using 7-(4-(1,5-dimethyl-1H-indazol-4-yl)cyclohex-3-en-1-yl)-3-methylpyrido[2,3-b]pyrazin-6(5H)-one (190 mg) as the starting material to give the crude product. The crude product was purified by TLC with the following conditions (PE:EA=3:1) to afford 7-((1r,4r)-4-(1,5-dimethyl-1H-indazol-4-yl)cyclohexyl)-3-methylpyrido[2,3-b]pyrazin-6(5H)-one (30 mg, 15.7%) as an off-white solid MS m/z: 388 [M+H]⁺.

Step 4: 7-((1r,4r)-4-(1,5-dimethyl-1H-indazol-4-yl)cyclohexyl)-3-methyl-5-((3-(trifluoromethyl)pyridin-2-yl)methyl)pyrido[2,3-b]pyrazin-6(5H)-one: Followed the General Procedure AP using 7-((1r,4r)-4-(1,5-dimethyl-1H-indazol-4-yl)cyclohexyl)-3-methylpyrido[2,3-b]pyrazin-6(5H)-one (20 mg, 0.052 mmol, 1.0 equiv.) and 2-(chloromethyl)-3-(trifluoromethyl)pyridine (12.1 mg, 0.062 mmol, 1.2 equiv.) as the starting materials to give 7-((1r,4r)-4-(1,5-dimethyl-1H-indazol-4-yl)cyclohexyl)-3-methyl-5-((3-(trifluoromethyl)pyridin-2-yl)methyl)pyrido[2,3-b]pyrazin-6(5H)-one (1.3 mg, 3.6%) as a white solid. ¹H NMR (400 MHz, Chloroform-d) δ 8.46 (d, J=4.9 Hz, 1H), 8.34 (s, 1H), 8.19 (s, 1H), 8.01 (d, J=7.6 Hz, 1H), 7.91 (s, 1H), 7.29 (s, 1H), 7.23 (s, 1H), 7.15 (d, J=8.5 Hz, 1H), 4.07 (s, 3H), 3.28-3.15 (m, 2H), 2.54 (s, 3H), 2.47 (s, 3H), 2.22 (d, J=10.9 Hz, 4H), 2.03 (d, J=14.7 Hz, 2H), 1.64 (dd, J=14.7, 11.0 Hz, 2H), 1.26 (d, J=2.4 Hz, 2H). MS m/z: 547.2 [M+H]⁺.

### 7-((1r,4r)-4-(2-Fluoro-6-methylphenyl) cyclohexyl)-3-methyl-5-((3-(2,2,2-trifluoroethyl) pyridin-2-yl)methyl) pyrido[2,3-b]pyrazin-6(5H)-one (219)

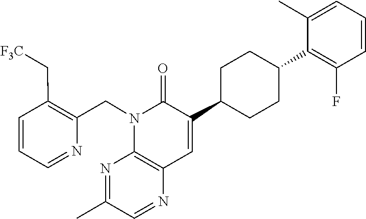 [SMILES: Cc1cnc2cc([C@H]3CC[C@H](c4c(C)cccc4F)CC3)c(=O)n(Cc3ncccc3CC(F)(F)F)c2n1]

Step 1: 1-(2-bromopyridin-3-yl)-2,2,2-trifluoroethan-1-ol: To a stirred solution of 2-bromopyridine-3-carbaldehyde (2 g, 10.7 mmol, 1 equiv) and CsF (2.45 g, 16.1 mmol, 1.5 equiv) in THF (20 mL) was added TMSCF₃ (2.29 g, 16.1 mmol, 1.5 equiv) at 0° C. The resulting mixture was stirred for 2 h at room temperature. The resulting mixture was extracted with EtOAc (3×100 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (1:3) to afford 1-(2-bromopyridin-3-yl)-2,2,2-trifluoroethan-1-ol (2 g, 72%) as a yellow oil. MS m/z: 256 [M+H]⁺.

Step 2: 1-(2-bromopyridin-3-yl)-2,2,2-trifluoroethyl methanesulfonate: Followed the General Procedure W using 1-(2-bromopyridin-3-yl)-2,2,2-trifluoroethan-1-ol (2 g, 7.81 mmol, 1 equiv), MsCl (1.78 g, 15.6 mmol, 2 equiv) as the starting materials to give 1-(2-bromopyridin-3-yl)-2,2,2-trifluoroethyl methanesulfonate (2 g, 76%) as a yellow oil. MS m/z: 334 [M+H]⁺.

Step 3: methyl 3-(2,2,2-trifluoro-1-((methylsulfonyl)oxy)ethyl)picolinate: Followed the General Procedure AI using 1-(2-bromopyridin-3-yl)-2,2,2-trifluoroethyl methanesulfonate (2 g, 6 mmol, 1 equiv) as the starting material to give methyl 3-(2,2,2-trifluoro-1-((methylsulfonyl)oxy)ethyl)picolinate (900 mg, 47%) as a yellow solid. MS m/z: 314 [M+H]⁺.

Step 4: methyl 3-(2,2,2-trifluoroethyl) picolinate: Followed the General Procedure AJ using methyl 3-(2,2,2-trifluoro-1-((methylsulfonyl)oxy)ethyl)picolinate (500 mg, 1.59 mmol, 1 equiv) as the starting material to give methyl 3-(2,2,2-trifluoroethyl) picolinate (180 mg, 51%) as a yellow oil. MS m/z: 220 [M+H]⁺.

Step 5: (3-(2,2,2-trifluoroethyl)pyridin-2-yl)methanol: Followed the General Procedure I using methyl 3-(2,2,2-trifluoroethyl) picolinate (180 mg, 0.821 mmol, 1 equiv) as the starting material to give (3-(2,2,2-trifluoroethyl)pyridin-2-yl)methanol (40 mg, 25%) as a colorless oil. MS m/z: 192 [M+H]⁺.

Step 6: 2-(chloromethyl)-3-(2,2,2-trifluoroethyl)pyridine: Followed the General Procedure J using (3-(2,2,2-trifluoroethyl)pyridin-2-yl)methanol (40 mg, 0.209 mmol, 1 equiv) as the starting material to give the crude product 2-(chloromethyl)-3-(2,2,2-trifluoroethyl)pyridine (30 mg) as a yellow solid. MS m/z: 210 [M+H]⁺.

Step 7: 7-((1r,4r)-4-(2-fluoro-6-methylphenyl)cyclohexyl)-3-methyl-5-((3-(2,2,2-trifluoroethyl) pyridin-2-yl)methyl) pyrido[2,3-b]pyrazin-6(5H)-one: Followed the General Procedure AP using 2-(chloromethyl)-3-(2,2,2-trifluoroethyl)pyridine (30 mg, 0.143 mmol, 1 equiv) and 7-((1r,4r)-4-(2-fluoro-6-methylphenyl)cyclohexyl)-3-methylpyrido[2,3-b]pyrazin-6(5H)-one (50.4 mg, 0.143 mmol, 1 equiv) as the starting materials to give 7-((1r,4r)-4-(2-fluoro-6-methylphenyl)cyclohexyl)-3-methyl-5-((3-(2,2,2-trifluoroethyl) pyridin-2-yl)methyl) pyrido[2,3-b]pyrazin-6(5H)-one (12.5 mg, 16.6%) as a white solid. ¹H NMR (400 MHz, Chloroform-d) δ 8.35 (d, J=4.3 Hz, 2H), 7.79 (s, 1H), 7.62 (d, J=7.7 Hz, 1H), 7.15 (dd, J=7.7, 4.7 Hz, 1H), 7.04-6.97 (m, 1H), 6.95-6.76 (m, 2H), 5.86 (s, 2H), 4.51-3.53 (m, 2H), 3.12 (tt, J=11.8, 2.9 Hz, 1H), 3.01-2.77 (m, 1H), 2.57 (s, 3H), 2.37 (s, 3H), 2.37-1.86 (m, 4H), 1.83 (d, 2H), 1.61-1.38 (m, 2H). MS m/z: 525.1 [M+H]⁺.

### 5-((3-Cyclopropylpyridin-2-yl)methyl)-7-((1r,4r)-4-(2-fluoro-6-methylphenyl)cyclohexyl)-3-methylpyrido[2,3-b]pyrazin-6(5H)-one (220)

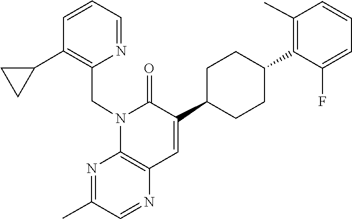 [SMILES: Cc1cnc2cc([C@H]3CC[C@H](c4c(C)cccc4F)CC3)c(=O)n(Cc3ncccc3C3CC3)c2n1]

Step 1: (3-cyclopropylpyridin-2-yl)methanol: Followed the General Procedure AQ using 3-cyclopropylpicolinic acid (300 mg, 1.84 mmol, 1 equiv.) as the starting materials to give (3-cyclopropylpyridin-2-yl)methano (30 mg, 10.9%) as a colorless oil. MS m/z: 150 [M+H]⁺.

Step 2: 2-(chloromethyl)-3-cyclopropylpyridine: Followed the General Procedure J using (3-cyclopropylpyridin-2-yl)methano (30 mg, 0.067 mmol, 1 equiv) as the starting materials to give the crude product 2-(chloromethyl)-3-cyclopropylpyridine (20 mg) as a colorless oil. MS m/z: 168 [M+H]⁺

Step 3: 5-((3-cyclopropylpyridin-2-yl)methyl)-7-((1r,4r)-4-(2-fluoro-6-methylphenyl)cyclohexyl)-3-methylpyrido[2,3-b]pyrazin-6(5H)-one: Followed the General Procedure AP using 2-(chloromethyl)-3-cyclopropylpyridine (20 mg, 0.119 mmol, 1 equiv.) and 7-((1r,4r)-4-(2-fluoro-6-methylphenyl)cyclohexyl)-3-methylpyrido[2,3-b]pyrazin-6(5H)-one (50.3 mg, 0.143 mmol, 1.2 equiv.) as the starting materials to give 5-((3-cyclopropylpyridin-2-yl)methyl)-7-((1r,4r)-4-(2-fluoro-6-methylphenyl)cyclohexyl)-3-methylpyrido[2,3-b]pyrazin-6(5H)-one (17.2 mg, 28.3%) as a white solid. ¹H NMR (400 MHz, Methanol-d₄) δ 8.41 (s, 1H), 8.04-8.00 (m, 1H), 7.89 (d, J=0.9 Hz, 1H), 7.57-7.53 (m, 1H), 7.18-7.14 (m, 1H), 7.08-7.01 (m, 1H), 6.96 (d, J=7.4 Hz, 1H), 6.84 (dd, J=12.1, 8.0 Hz, 1H), 6.06 (s, 2H), 3.11-3.00 (m, 2H), 2.52 (s, 3H), 2.39 (s, 3H), 2.20-2.10 (m, 5H), 1.86 (d, J=12.4 Hz, 2H), 1.63 (d, J=12.1 Hz, 2H), 1.16-1.11 (m, 2H), 0.88-0.83 (m, 2H). MS m/z: 483.2 [M+H]⁺.

### 5-((3-Cyclopropylpyrazin-2-yl)methyl)-7-((1r,4r)-4-(2-fluoro-6-methylphenyl)cyclohexyl)-3-methylpyrido[2,3-b]pyrazin-6(5H)-one (221)

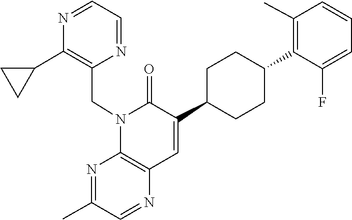 [SMILES: Cc1cnc2cc([C@H]3CC[C@H](c4c(C)cccc4F)CC3)c(=O)n(Cc3nccnc3C3CC3)c2n1]

Step 1: (3-cyclopropylpyrazin-2-yl)methanol: Followed the General Procedure AL using (3-chloropyrazin-2-yl)methanol (500 mg, 3.45 mmol, 1 equiv.) and cyclopropylboronic acid (891 mg, 10.377 mmol, 3.0 equiv.) as the starting materials to give (3-cyclopropylpyrazin-2-yl)methanol (167 mg, 32%) as a light brown oil. MS m/z: 151 [M+H]⁺.

Step 2: 2-(chloromethyl)-3-cyclopropylpyrazine: Followed the General Procedure J using (3-cyclopropylpyrazin-2-yl)methanol (150 mg, 0.999 mmol, 1 equiv.) as the starting material to give the crude product 2-(chloromethyl)-3-cyclopropylpyrazine (100 mg) as a colorless oil. MS m/z: 169 [M+H]+

Step 3: 5-((3-cyclopropylpyrazin-2-yl)methyl)-7-((1r,4r)-4-(2-fluoro-6-methylphenyl)cyclohexyl)-3-methylpyrido[2,3-b]pyrazin-6(5H)-one: Followed the General Procedure AP using 2-(chloromethyl)-3-cyclopropylpyrazine (50 mg, 0.142 mmol, 1 equiv.) and 7-((1r,4r)-4-(2-fluoro-6-methylphenyl)cyclohexyl)-3-methylpyrido[2,3-b]pyrazin-6(5H)-one (125 mg, 0.170 mmol, 1.2 equiv.) as the starting materials to give 5-((3-cyclopropylpyrazin-2-yl)methyl)-7-((1r,4r)-4-(2-fluoro-6-methylphenyl)cyclohexyl)-3-methylpyrido[2,3-b]pyrazin-6(5H)-one (27.3 mg, 39.0%) as a white solid. ¹H NMR (400 MHz, Chloroform-d) δ 8.34 (s, 1H), 8.24 (d, J=2.6 Hz, 1H), 8.07 (d, J=2.6 Hz, 1H), 7.85 (d, J=0.9 Hz, 1H), 7.07-6.99 (m, 1H), 6.95-6.82 (m, 2H), 6.06 (s, 2H), 3.15 (t, J=12.1 Hz, 1H), 2.91 (t, J=12.3 Hz, 1H), 2.55 (s, 3H), 2.38 (s, 3H), 2.36-2.30 (m, 1H), 2.16 (t, J=11.3 Hz, 4H), 1.88-1.82 (m, 2H), 1.54 (d, J=12.5 Hz, 2H), 1.30-1.18 (m, 4H). MS m/z: 484.2 [M+H]⁺.

### 7-((1r,4r)-4-(2,6-Difluorophenyl)cyclohexyl)-3-methyl-5-((3-(trifluoromethyl)pyridin-2-yl)methyl)pyrido[2,3-b]pyrazin-6(5H)-one (222)

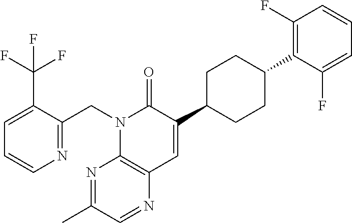 [SMILES: Cc1cnc2cc([C@H]3CC[C@H](c4c(F)cccc4F)CC3)c(=O)n(Cc3ncccc3C(F)(F)F)c2n1]

Step 1: ethyl 2-(2′,6′-difluoro-2,3,4,5-tetrahydro-[1,1′-biphenyl]-4-yl)acetate: Followed the General Procedure AL using ethyl 2-(4-(4,4,5,5-tetramethyl-1,3,2-dioxaborolan-2-yl)cyclohex-3-en-1-yl)acetate (1 g, 3.4 mmol, 1 equiv) and 2-bromo-1,3-difluorobenzene (0.72 g, 3.74 mmol, 1.1 equiv) as the starting materials to give ethyl 2-(2′,6′-difluoro-2,3,4,5-tetrahydro-[1,1′-biphenyl]-4-yl)acetate (800 mg, 84%) as a yellow oil. MS m/z: 281[M+H]⁺.

Step 2: ethyl 2-(4-(2,6-difluorophenyl)cyclohexyl)acetate: Followed the General Procedure AR using ethyl 2-(2′,6′-difluoro-2,3,4,5-tetrahydro-[1,1′-biphenyl]-4-yl)acetate (800 mg, 2.854 mmol, 1 equiv) as the starting material to give the crude product ethyl 2-(4-(2,6-difluorophenyl)cyclohexyl)acetate (700 mg) as a colorless oil. MS m/z: 283 [M+H]⁺.

Step 3: 2-((1r,4r)-4-(2,6-difluorophenyl)cyclohexyl)acetic acid: To a stirred solution of ethyl 2-(4-(2,6-difluorophenyl)cyclohexyl)acetate (700 mg, 2.48 mmol, 1 equiv) in EtOH (10 mL) and H₂O (10 mL) was added LiOH (356 mg, 14.8 mmol, 6 equiv) at room temperature. The resulting mixture was stirred for overnight at 50° C. The mixture was acidified to pH 3 with conc. HCl. The resulting mixture was extracted with EtOAc (3×100 mL). The combined organic layers were washed with brine (1×100 mL), dried over anhydrous Na₂SO₄. After filtration, the filtrate was concentrated under reduced pressure to give the crude product 2-((1r,4r)-4-(2,6-difluorophenyl)cyclohexyl)acetic acid (300 mg) as a white solid. MS m/z: 255 [M+H]⁺.

Step 4: ethyl 2-((1r,4r)-4-(2,6-difluorophenyl)cyclohexyl)acetate: To a stirred solution of 2-((1r,4r)-4-(2,6-difluorophenyl)cyclohexyl)acetic acid (300 mg, 1.180 mmol, 1 equiv) in EtOH (10 mL) was added H₂SO₄ (347 mg, 3.54 mmol, 3 equiv) dropwise at room temperature. The resulting mixture was stirred for overnight at 60° C. The resulting mixture was extracted with EtOAc (3×100 mL). The combined organic layers were washed with brine (1×100 mL), dried over anhydrous Na₂SO₄. After filtration, the filtrate was concentrated under reduced pressure to give the crude product ethyl 2-((1r,4r)-4-(2,6-difluorophenyl)cyclohexyl)acetate (150 mg) as a colorless oil. MS m/z: 283 [M+H]⁺.

Step 5: 7-((1r,4r)-4-(2,6-difluorophenyl)cyclohexyl)-3-methylpyrido[2,3-b]pyrazin-6(5H)-one: Followed the General Procedure D using ethyl 2-((1r,4r)-4-(2,6-difluorophenyl)cyclohexyl)acetate (150 mg, 0.531 mmol, 1 equiv) and 3-amino-5-methylpyrazine-2-carbaldehyde (80.15 mg, 0.584 mmol, 1.1 equiv) as the starting materials to give 7-((1r,4r)-4-(2,6-difluorophenyl)cyclohexyl)-3-methylpyrido[2,3-b]pyrazin-6(5H)-one (50 mg, 26%) as a yellow solid. MS m/z: 356 [M+H]⁺.

Step 6: 7-((1r,4r)-4-(2,6-difluorophenyl)cyclohexyl)-3-methyl-5-((3-(trifluoromethyl)pyridin-2-yl)methyl)pyrido[2,3-b]pyrazin-6(5H)-one: Followed the General Procedure AP using 7-((1r,4r)-4-(2,6-difluorophenyl)cyclohexyl)-3-methylpyrido[2,3-b]pyrazin-6(5H)-one (50 mg, 0.141 mmol, 1 equiv) and 2-(chloromethyl)-3-(trifluoromethyl)pyridine (39.1 mg, 0.169 mmol, 1.2 equiv) as the starting materials to give 7-((1r,4r)-4-(2,6-difluorophenyl)cyclohexyl)-3-methyl-5-((3-(trifluoromethyl)pyridin-2-yl)methyl)pyrido[2,3-b]pyrazin-6(5H)-one (24.6 mg, 33.9%) as a white solid. ¹H NMR (400 MHz, Chloroform-d) δ 8.48-8.42 (m, 1H), 8.33 (s, 1H), 8.03-7.96 (m, 1H), 7.85 (s, 1H), 7.24 (s, 1H), 7.17-7.05 (m, 1H), 6.82 (t, J=8.6 Hz, 2H), 6.04 (s, 2H), 3.12 (q, J=11.8 Hz, 2H), 2.53 (s, 3H), 2.13 (t, J=13.0 Hz, 4H), 1.91 (s, 2H), 2.61-2.45 (m, 2H). MS m/z: 515.20 [M+H]⁺.

### 3-((1r,4r)-4-(2,6-Difluorophenyl)cyclohexyl)-7-methyl-1-((3-(trifluoromethyl)pyridin-2-yl)methyl)-1,8-naphthyridin-2(1H)-one (223)

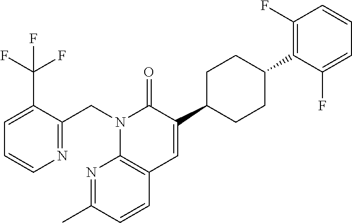 [SMILES: Cc1ccc2cc([C@H]3CC[C@H](c4c(F)cccc4F)CC3)c(=O)n(Cc3ncccc3C(F)(F)F)c2n1]

Step 1: 3-((1r,4r)-4-(2,6-difluorophenyl)cyclohexyl)-7-methyl-1-((3-(trifluoromethyl)pyridin-2-yl)methyl)-1,8-naphthyridin-2(1H)-one: Followed the General Procedure AP using 3-((1r,4r)-4-(2,6-difluorophenyl)cyclohexyl)-7-methyl-1,8-naphthyridin-2(1H)-one (100 mg, 0.282 mmol, 1 equiv) and 2-(chloromethyl)-3-(trifluoromethyl)pyridine (78.5 mg, 0.338 mmol, 1.2 equiv) as the starting materials to give 3-((1r,4r)-4-(2,6-difluorophenyl)cyclohexyl)-7-methyl-1-((3-(trifluoromethyl)pyridin-2-yl)methyl)-1,8-naphthyridin-2(1H)-one (61.7 mg, 41.7%) as a white solid. ¹H NMR (400 MHz, Chloroform-d) δ 8.47 (d, J=4.7 Hz, 1H), 7.98 (d, J=7.8 Hz, 1H), 7.75 (d, J=7.8 Hz, 1H), 7.53 (s, 1H), 7.25-7.20 (m, 1H), 7.16-7.04 (m, 1H), 6.99 (d, J=7.8 Hz, 1H), 6.82 (t, J=8.6 Hz, 2H), 6.12 (s, 2H), 3.18-3.05 (m, 2H), 2.47 (s, 3H), 2.18-2.04 (m, 4H), 1.92-1.84 (m, 2H), 1.59-1.44 (m, 2H). MS m/z: 514.1 [M+H]⁺.

### 7-((1r,4r)-4-(2-Fluoro-6-methylphenyl)cyclohexyl)-3-methyl-5-(2-(trifluoromethyl)benzyl)pyrido[2,3-b]pyrazin-6(5H)-one (224)

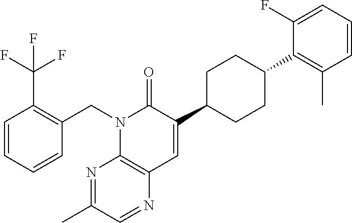 [SMILES: Cc1cnc2cc([C@H]3CC[C@H](c4c(C)cccc4F)CC3)c(=O)n(Cc3ccccc3C(F)(F)F)c2n1]

Step 1: 7-((1r,4r)-4-(2-fluoro-6-methylphenyl)cyclohexyl)-3-methyl-5-(2-(trifluoromethyl)benzyl)pyrido[2,3-b]pyrazin-6(5H)-one: Followed the General Procedure AP using 7-((1r,4r)-4-(2-fluoro-6-methylphenyl)cyclohexyl)-3-methylpyrido[2,3-b]pyrazin-6(5H)-one (30 mg, 0.085 mmol, 1 equiv.) and 1-(bromomethyl)-2-(trifluoromethyl)benzene (24.5 mg, 0.102 mmol, 1.2 equiv.) as the starting materials to give 7-((1r,4r)-4-(2-fluoro-6-methylphenyl)cyclohexyl)-3-methyl-5-(2-(trifluoromethyl)benzyl)pyrido[2,3-b]pyrazin-6(5H)-one (22.8 mg, 48.8%) as a white solid. ¹H NMR (400 MHz, Chloroform-d) δ 8.34 (s, 1H), 7.90 (s, 1H), 7.75-7.68 (m, 1H), 7.37-7.30 (m, 2H), 7.09-6.99 (m, 1H), 6.95-6.90 (m, 1H), 6.89-6.82 (m, 1H), 6.75-6.69 (m, 1H), 5.93 (s, 2H), 3.18 (t, J=12.0 Hz, 1H), 2.97-2.86 (m, 1H), 2.55 (s, 3H), 2.38 (s, 3H), 2.22-2.11 (m, 4H), 1.91-1.88 (m, 1H), 1.88-1.86 (m, 1H), 1.62-1.49 (m, 2H). MS m/z: 510.05 [M+H]⁺.

### 3-((1r,4r)-4-(2-Fluoro-6-methylphenyl)cyclohexyl)-7-methyl-1-(2-(trifluoromethyl)benzyl)-1,8-naphthyridin-2(1H)-one (225)

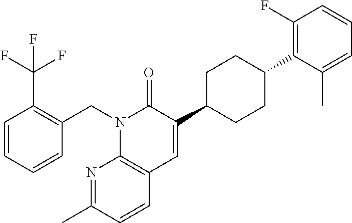 [SMILES: Cc1ccc2cc([C@H]3CC[C@H](c4c(C)cccc4F)CC3)c(=O)n(Cc3ccccc3C(F)(F)F)c2n1]

Step 1: 3-((1r,4r)-4-(2-fluoro-6-methylphenyl)cyclohexyl)-7-methyl-1-(2-(trifluoromethyl) benzyl)-1,8-naphthyridin-2(1H)-one: Followed the General Procedure AP using 3-((1r,4r)-4-(2-fluoro-6-methylphenyl) cyclohexyl)-7-methyl-1,8-naphthyridin-2(1H)-one (50 mg, 0.14 mmol, 1 equiv) and 1-(bromomethyl)-2-(trifluoromethyl)benzene (40.9 mg, 0.168 mmol, 1.2 equiv) as the starting materials to give 3-((1r,4r)-4-(2-fluoro-6-methylphenyl)cyclohexyl)-7-methyl-1-(2-(trifluoromethyl) benzyl)-1,8-naphthyridin-2(1H)-one (27.6 mg, 38%) as a white solid. ¹H NMR (400 MHz, Chloroform-d) δ 7.76 (d, J=7.8 Hz, 1H), 7.70 (d, J=5.0 Hz, 1H), 7.55 (s, 1H), 7.30-7.27 (m, 2H), 7.02 (d, J=7.8 Hz, 2H), 6.92 (d, J=7.4 Hz, 1H), 6.88-6.82 (m, 1H), 6.73-6.71 (m, 1H), 6.00 (s, 2H), 3.15 (s, 1H), 2.92 (d, J=12.5 Hz, 1H), 2.49 (s, 3H), 2.37 (s, 3H), 2.20-2.13 (m, 4H), 1.85 (d, J=12.8 Hz, 2H), 1.59-1.52 (m, 2H). MS m/z: 509.10.[M+H]⁺.

### 2-(2,2-Difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (226)

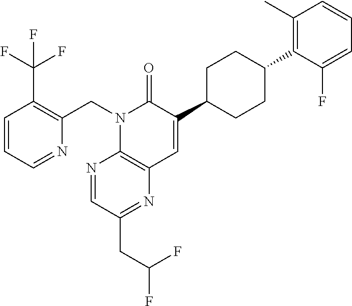 [SMILES: Cc1cccc(F)c1[C@H]1CC[C@H](c2cc3nc(CC(F)F)cnc3n(Cc3ncccc3C(F)(F)F)c2=O)CC1]

Step 1: 2-bromo-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AP using 2-bromo-7-((1r,4r)-4-(2-fluoro-6-methylphenyl)cyclohexyl)pyrido[2,3-b]pyrazin-6(5H)-one (80 mg, 0.192 mmol, 1 equiv) and 2-(chloromethyl)-3-(trifluoromethyl)pyridine (48.8 mg, 0.250 mmol, 1.3 equiv) as the starting materials to give 2-bromo-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (60 mg, 50%) as a white solid. MS m/z: 575 [M+H]⁺.

Step 2: (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-2-yl)boronic acid: To a stirred mixture of 2-bromo-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (100 mg, 0.174 mmol, 1 equiv) and (BPin)₂ (137 mg, 0.522 mmol, 3 equiv) in dioxane (2 mL) were added KOAc (34.1 mg, 0.348 mmol, 2 equiv) and Pd(dppf)Cl₂ (25.43 mg, 0.035 mmol, 0.2 equiv). The resulting mixture was stirred for 2 h at 80° C. under argon atmosphere. The resulting mixture was used in the next step directly without further purification. MS m/z: 541 [M+H]⁺.

Step 3: 2-(2,2-difluorovinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AL using (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-2-yl)boronic acid (100 mg, 0.185 mmol, 1 equiv) and 2-bromo-1,1-difluoroethene (52.9 mg, 0.370 mmol, 2 equiv) as the starting materials to give 2-(2,2-difluorovinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (20 mg, 19%) as a white solid. MS m/z: 559 [M+H]⁺.

Step 4: 2-(2,2-difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AJ using 2-(2,2-difluorovinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (100 mg, 0.179 mmol, 1 equiv) as the starting material to give 2-(2,2-difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (6.6 mg, 5.8%) as a white solid. ¹H NMR (300 MHz, Chloroform-d) δ 8.44 (d, J=4.8 Hz, 1H), 8.32 (s, 1H), 8.00 (d, J=7.9 Hz, 1H), 7.84 (s, 1H), 7.28-7.22 (m, 1H), 7.11-7.02 (m, 1H), 6.97-6.82 (m, 2H), 6.49-6.21 (m, 1H), 6.05 (s, 2H), 3.57-3.37 (m, 2H), 3.19 (t, J=12.0 Hz, 1H), 2.93 (t, J=12.1 Hz, 1H), 2.40 (s, 3H), 2.19 (d, J=12.0 Hz, 4H), 1.88 (d, J=12.8 Hz, 2H), 1.50 (s, 1H). MS m/z: 561.1 [M+H]⁺.

### 7-((1r,4r)-4-(2-Fluoro-6-methylphenyl)cyclohexyl)-3-propyl-5-((3-(trifluoromethyl)pyridin-2-yl)methyl)pyrido[2,3-b]pyrazin-6(5H)-one (227)

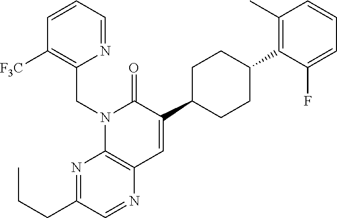 [SMILES: CCCc1cnc2cc([C@H]3CC[C@H](c4c(C)cccc4F)CC3)c(=O)n(Cc3ncccc3C(F)(F)F)c2n1]

Step 1: 7-((1r,4r)-4-(2-fluoro-6-methylphenyl)cyclohexyl)-3-((Z)-prop-1-en-1-yl)-5-((3-(trifluoromethyl)pyridin-2-yl)methyl)pyrido[2,3-b]pyrazin-6(5H)-one: Followed the General Procedure AL using (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-3-yl)boronic acid (60 mg, 0.111 mmol, 1 equiv) and (E)-1-bromoprop-1-ene (26.87 mg, 0.222 mmol, 2 equiv) as the starting materials to give 7-((1r,4r)-4-(2-fluoro-6-methylphenyl)cyclohexyl)-3-((Z)-prop-i-en-1-yl)-5-((3-(trifluoromethyl)pyridin-2-yl)methyl)pyrido[2,3-b]pyrazin-6(5H)-one (30 mg, 50%) as a white solid. MS m/z: 537 [M+H]⁺.

Step 2: 7-((1r,4r)-4-(2-fluoro-6-methylphenyl)cyclohexyl)-3-propyl-5-((3-(trifluoromethyl)pyridin-2-yl)methyl)pyrido[2,3-b]pyrazin-6(5H)-one: Followed the General Procedure AJ using 7-((1r,4r)-4-(2-fluoro-6-methylphenyl)cyclohexyl)-3-((Z)-prop-1-en-1-yl)-5-((3-(trifluoromethyl)pyridin-2-yl)methyl)pyrido[2,3-b]pyrazin-6(5H)-one (30 mg, 0.056 mmol, 1 equiv) as the starting material to give 7-((1r,4r)-4-(2-fluoro-6-methylphenyl)cyclohexyl)-3-propyl-5-((3-(trifluoromethyl)pyridin-2-yl)methyl)pyrido[2,3-b]pyrazin-6(5H)-one (13.1 mg, 42.3%) as a white solid. ¹H NMR (400 MHz, Chloroform-d) δ 8.44-8.38 (m, 1H), 8.29 (s, 1H), 8.00-7.93 (m, 1H), 7.83 (s, 1H), 7.24-7.20 (m, 1H), 7.08-6.98 (m, 1H), 6.92 (d, J=7.4 Hz, 1H), 6.89-6.80 (m, 1H), 6.04 (s, 2H), 3.23-3.11 (m, 1H), 2.91 (t, J=12.6 Hz, 1H), 2.72 (t, J=7.4 Hz, 2H), 2.38 (s, 3H), 2.23-2.11 (m, 4H), 1.88-1.82 (m, 2H), 1.66-1.51 (m, 4H), 0.78 (t, J=7.4 Hz, 3H). MS m/z: 539.15 [M+H]⁺.

### 7-((1r,4r)-4-(2-Fluoro-6-methylphenyl)cyclohexyl)-2-(2,2,2-trifluoroethyl)-5-((3-(trifluoromethyl)pyridin-2-yl)methyl)pyrido[2,3-b]pyrazin-6(5H)-one (228)

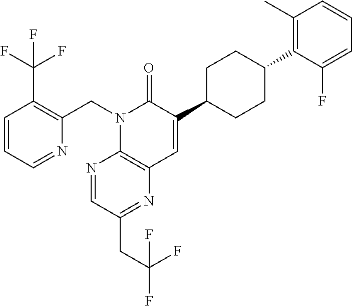 [SMILES: Cc1cccc(F)c1[C@H]1CC[C@H](c2cc3nc(CC(F)(F)F)cnc3n(Cc3ncccc3C(F)(F)F)c2=O)CC1]

Step 1: 7-((1r,4r)-4-(2-fluoro-6-methylphenyl)cyclohexyl)-2-(2,2,2-trifluoroethyl)-5-((3-(trifluoromethyl)pyridin-2-yl)methyl)pyrido[2,3-b]pyrazin-6(5H)-one: Followed the General Procedure AL using (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-2-yl)boronic acid (15.6 mg, 0.096 mmol, 1.3 equiv) and 2-bromo-1,1,1-trifluoroethane (gas) as the starting materials to give 7-((1r,4r)-4-(2-fluoro-6-methylphenyl)cyclohexyl)-2-(2,2,2-trifluoroethyl)-5-((3-(trifluoromethyl)pyridin-2-yl)methyl)pyrido[2,3-b]pyrazin-6(5H)-one (5.5 mg, 11.9%) as an off-white solid. ¹H NMR (400 MHz, Chloroform-d) δ 8.46-8.41 (m, 1H), 8.36 (s, 1H), 8.02-7.97 (m, 1H), 7.85 (d, J=0.9 Hz, 1H), 7.30-7.20 (m, 1H), 7.04 (td, J=7.8, 5.5 Hz, 1H), 6.92 (d, J=7.4 Hz, 1H), 6.89-6.81 (m, 1H), 6.03 (s, 2H), 3.69 (q, J=10.5 Hz, 2H), 3.16 (t, J=12.1 Hz, 1H), 2.90 (t, J=12.2 Hz, 1H), 2.37 (s, 3H), 2.16 (d, J=12.2 Hz, 4H), 1.86 (d, J=12.2 Hz, 2H), 1.53 (t, J=12.7 Hz, 2H). MS m/z: 579.1 [M+H]⁺.

### 7-((1r,4r)-4-(2-Fluoro-6-methylphenyl)cyclohexyl)-3-(2,2,2-trifluoroethyl)-5-((3-(trifluoromethyl)pyridin-2-yl)methyl)pyrido[2,3-b]pyrazin-6(5H)-one (229)

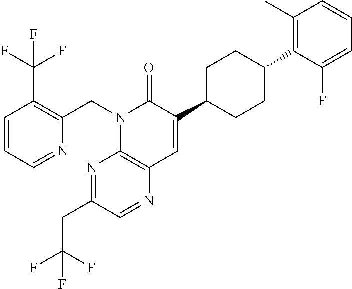 [SMILES: Cc1cccc(F)c1[C@H]1CC[C@H](c2cc3ncc(CC(F)(F)F)nc3n(Cc3ncccc3C(F)(F)F)c2=O)CC1]

Step 1: (7-((1r,4r)-4-(2-fluoro-6-methylphenyl) cyclohexyl)-6-oxo-5-((3-(trifluoromethyl) pyridin-2-yl) methyl)-5,6-dihydropyrido[2,3-b]pyrazin-3-yl)boronic acid: To a stirred mixture of 3-chloro-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (100 mg, 0.188 mmol, 1 equiv) and (BPin)₂ (95.6 mg, 0.376 mmol, 2 equiv) in dioxane (2 mL) were added KOAc (34.1 mg, 0.348 mmol, 2 equiv) and Pd(dppf)Cl₂ (25.43 mg, 0.035 mmol, 0.2 equiv). The resulting mixture was stirred for 2 h at 80° C. under argon atmosphere. The resulting mixture was used in the next step directly without further purification. MS m/z: 541 [M+H]⁺.

Step 2: 7-((1r,4r)-4-(2-fluoro-6-methylphenyl)cyclohexyl)-3-(2,2,2-trifluoroethyl)-5-((3-(trifluoromethyl)pyridin-2-yl)methyl)pyrido[2,3-b]pyrazin-6(5H)-one: Followed the General Procedure AL using (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-2-yl)boronic acid (15.6 mg, 0.096 mmol, 1.3 equiv) and 2-bromo-1,1,1-trifluoroethane (gas) as the starting materials to give 7-((1r,4r)-4-(2-fluoro-6-methylphenyl)cyclohexyl)-3-(2,2,2-trifluoroethyl)-5-((3-(trifluoromethyl)pyridin-2-yl)methyl)pyrido[2,3-b]pyrazin-6(5H)-one (13.4 mg, 15%) as a white solid. ¹H NMR (400 MHz, Chloroform-d) δ 8.43 (s, 1H), 8.40 (dd, J=5.0, 1.6 Hz, 1H), 7.97 (dd, J=8.0, 1.6 Hz, 1H), 7.84 (d, J=0.9 Hz, 1H), 7.23 (dd, J=7.9, 4.9 Hz, 1H), 7.08-7.00 (m, 1H), 6.92 (d, J=7.4 Hz, 1H), 6.90-6.80 (m, 1H), 6.01 (s, 2H), 3.53 (q, J=10.4 Hz, 2H), 3.24-3.14 (m, 1H), 2.92 (t, J=12.3 Hz, 1H), 2.38 (s, 3H), 2.25-2.11 (m, 4H), 1.94-1.82 (m, 2H), 1.59-1.50 (m, 2H). MS m/z: 579.05 [M+H]⁺.

### 7-((1r,4r)-4-(2-Methoxy-4-methylpyridin-3-yl)cyclohexyl)-3-methyl-5-((3-(trifluoromethyl) pyridine-2-yl)methyl)pyrido[2,3-b]pyrazin-6(5H)-one (230)

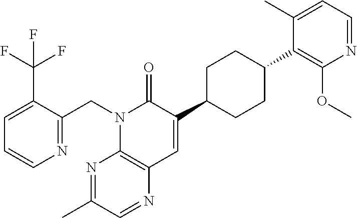 [SMILES: COc1nccc(C)c1[C@H]1CC[C@H](c2cc3ncc(C)nc3n(Cc3ncccc3C(F)(F)F)c2=O)CC1]

Step 1: ethyl 2-(4-(2-methoxy-4-methylpyridin-3-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (500 mg, 1.70 mmol, 1 equiv) and 3-bromo-2-methoxy-4-methylpyridine (412.07 mg, 2.04 mmol, 1.2 equiv) as the starting materials to give ethyl 2-[4-(2-methoxy-4-methylpyridin-3-yl)cyclohex-3-en-1-yl]acetate (400 mg, 81%) as a light yellow oil. MS m/z: 290 [M+H]⁺.

Step 2: ethyl 2-(4-(2-methoxy-4-methylpyridin-3-yl)cyclohexyl)acetate: Followed the General Procedure AR using ethyl 2-(4-(2-methoxy-4-methylpyridin-3-yl)cyclohex-3-en-1-yl)acetate (400 mg, 1.382 mmol, 1 equiv) as the starting material to give the crude product ethyl 2-(4-(2-methoxy-4-methylpyridin-3-yl)cyclohexyl)acetate (300 mg) as a colorless oil. MS m/z: 292 [M+H]⁺.

Step 3: 7-(4-(2-methoxy-4-methylpyridin-3-yl)cyclohexyl)-3-methylpyrido[2,3-b]pyrazin-6(5H)-one: Followed the General Procedure D using ethyl 2-(4-(2-methoxy-4-methylpyridin-3-yl)cyclohexyl)acetate (300 mg, 1.03 mmol, 1 equiv) and 3-amino-5-methylpyrazine-2-carbaldehyde (169 mg, 1.24 mmol, 1.2 equiv) as the starting materials to give 7-(4-(2-methoxy-4-methylpyridin-3-yl)cyclohexyl)-3-methylpyrido[2,3-b]pyrazin-6(5H)-one (150 mg, 40%) as a yellow solid. MS m/z: 365 [M+H]⁺.

Step 4: 7-((1r,4r)-4-(2-methoxy-4-methylpyridin-3-yl)cyclohexyl)-3-methyl-5-((3-(trifluoromethyl) pyridin-2-yl)methyl) pyrido[2,3-b]pyrazin-6(5H)-one: Followed the General Procedure AP using 7-(4-(2-methoxy-4-methylpyridin-3-yl)cyclohexyl)-3-methylpyrido[2,3-b]pyrazin-6(5H)-one (150 mg, 0.412 mmol, 1 equiv) and 2-(chloromethyl)-3-(trifluoromethyl)pyridine (96.6 mg, 0.494 mmol, 1.2 equiv) as the starting materials to give the crude product ethyl 2-(4-(2-methoxy-4-methylpyridin-3-yl)cyclohexyl)acetate (90 mg) as a yellow solid. The crude product was purified by Prep-HPLC with the following conditions (Column: CHIRALPAK IC, 2*25 cm, 5 m; Mobile Phase A: Hex (10 mM NH₃-MeOH), Mobile Phase B: EtOH-HPLC; Flow rate: 20 mL/min; Gradient: isocratic 10; Wave Length: 210/220 nm; RT1 (min): 10.2; RT2 (min): 13.8; Sample Solvent: EtOH:DCM=2:1; Injection Volume: 0.5 mL; Number Of Runs: 9) to afford 7-((1r,4r)-4-(2-methoxy-4-methylpyridin-3-yl)cyclohexyl)-3-methyl-5-((3-(trifluoromethyl) pyridin-2-yl)methyl) pyrido[2,3-b]pyrazin-6(5H)-one (46 mg, 50.7%) as a white solid. ¹H NMR (400 MHz, Chloroform-d) δ 8.45-8.42 (m, 1H), 8.33 (s, 1H), 7.99-7.95 (m, 1H), 7.88 (d, J=5.2 Hz, 1H), 7.81 (s, 1H), 7.25-7.21 (m, 1H), 6.69 (d, J=5.2 Hz, 1H), 6.04 (s, 2H), 3.94 (s, 3H), 3.17-3.10 (m, 1H), 2.94 (s, 1H), 2.52 (s, 3H), 2.35 (s, 5H), 2.16-2.10 (m, 2H), 1.71-1.66 (m, 2H), 1.55-1.47 (m, 2H). MS m/z: 524.1 [M+H]⁺.

### 3-(2,2-Difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (231)

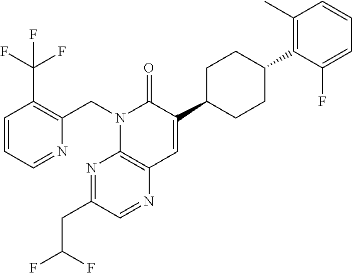 [SMILES: Cc1cccc(F)c1[C@H]1CC[C@H](c2cc3ncc(CC(F)F)nc3n(Cc3ncccc3C(F)(F)F)c2=O)CC1]

Step 1: 3-chloro-7-((1r,4r)-4-(2-fluoro-6-methylphenyl)cyclohexyl)pyrido[2,3-b]pyrazin-6(5H)-one: Followed the General Procedure D using 3-amino-5-chloropyrazine-2-carbaldehyde (500 mg, 3.17 mmol, 1 equiv) and ethyl 2-((1r,4r)-4-(2-fluoro-6-methylphenyl)cyclohexyl)acetate (883 mg, 3.17 mmol, 1 equiv) as the starting material to give 3-chloro-7-((1r,4r)-4-(2-fluoro-6-methylphenyl)cyclohexyl)pyrido[2,3-b]pyrazin-6(5H)-one (150 mg, 44%) as a yellow solid. MS m/z: 372 [M+H]⁺.

Step 2: 3-chloro-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AP using 3-chloro-7-((1r,4r)-4-(2-fluoro-6-methylphenyl)cyclohexyl)pyrido[2,3-b]pyrazin-6(5H)-one (150 mg, 0.403 mmol, 1 equiv) and 2-(chloromethyl)-3-(trifluoromethyl)pyridine (112 mg, 0.484 mmol, 1.2 equiv) as the starting materials to give 3-chloro-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (140 mg, 65%) as a white solid. MS m/z: 531 [M+H]⁺.

Step 3: (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-3-yl)boronic acid: To a stirred mixture of 3-chloro-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (140 mg, 0.264 mmol, 1 equiv) and (BPin)₂ (208 mg, 0.792 mmol, 3 equiv) in dioxane (2 mL) were added KOAc (51.7 mg, 0.528 mmol, 2 equiv) and Pd(dppf)Cl₂ (19.3 mg, 0.0264 mmol, 0.1 equiv). The resulting mixture was stirred for 2 h at 80° C. under argon atmosphere. The resulting mixture was used in the next step directly without further purification. MS m/z: 541 [M+H]⁺.

Step 4: 3-(2,2-difluorovinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AL using (7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazin-3-yl)boronic acid (140 mg, 0.259 mmol, 1 equiv) and 2-bromo-1,1-difluoroethene (gas) as the starting materials to give 3-(2,2-difluorovinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (40 mg, 27%) as a white solid. MS m/z: 559 [M+H]⁺.

Step 5: 3-(2,2-difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AJ using 3-(2,2-difluorovinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (40 mg, 0.072 mmol, 1 equiv) as the starting material to give 3-(2,2-difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (6.9 mg, 16.5%) as a white solid. ¹H NMR (400 MHz, Chloroform-d) δ 8.44 (d, J=4.5 Hz, 1H), 8.37 (s, 1H), 8.01 (d, J=7.7 Hz, 1H), 7.85 (s, 1H), 7.29 (s, 1H), 7.09-6.99 (m, 1H), 6.92 (d, J=7.5 Hz, 1H), 6.89-6.80 (m, 1H), 6.01 (s, 2H), 5.97 (t, J=4.6 Hz, 1H), 3.36-3.23 (m, 2H), 3.17 (t, J=12.0 Hz, 1H), 2.90 (d, J=12.4 Hz, 1H), 2.38 (s, 3H), 2.17 (d, J=11.9 Hz, 4H), 1.89-1.85 (m, 2H), 1.57 (t, J=12.2 Hz, 2H). MS m/z: 561.1 [M+H]⁺.

### 5-((3-(2,2-Difluoroethyl)pyridin-2-yl)methyl)-7-((1r,4r)-4-(2-fluoro-6-methylphenyl)cyclohexyl)-3-methylpyrido[2,3-b]pyrazin-6(5H)-one (232)

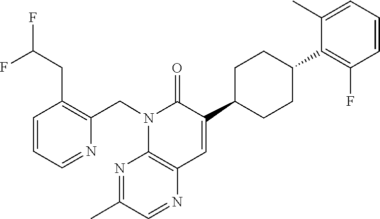 [SMILES: Cc1cnc2cc([C@H]3CC[C@H](c4c(C)cccc4F)CC3)c(=O)n(Cc3ncccc3CC(F)F)c2n1]

Step 1: methyl 3-vinylpicolinate: A mixture of methyl 3-bromopicolinate (1000 mg, 4.63 mmol, 1.0 equiv.), 4,4,5,5-tetramethyl-2-vinyl-1,3,2-dioxaborolane (780 mg, 5.09 mmol, 1.1 equiv.), Pd(dppf)Cl₂ (0.34 g, 0.463 mmol, 0.1 equiv) and K₂CO₃ (1920 mg, 13.9 mmol, 3.0 equiv.) in 1,4-dioxane (10 mL) and H₂O (2 mL) was stirred for 1 h at 80° C. under argon atmosphere. The resulting mixture was extracted with EtOAc (3×100 mL). The combined organic layers were washed with brine (1×200 mL), dried over anhydrous Na₂SO₄. After filtration, the filtrate was concentrated under reduced pressure. The residue was purified by silica gel column chromatography, eluted with PE/EA (1:1) to afford methyl 3-vinylpicolinate (600 mg, 79%) as a light yellow solid. MS m/z: 164 [M+H]⁺.

Step 2: methyl 3-formylpicolinate: A mixture of methyl 3-vinylpicolinate (600 mg, 3.66 mmol, 1.0 equiv.), K₂OsO₄.2H₂O (135 mg, 0.366 mmol, 0.1 equiv.) and NaIO₄ (2360 mg, 11.0 mmol, 3 equiv.) in THF (5 mL) and H₂O (5 mL) was stirred for 2 h at room temperature. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by Prep-TLC (PE/EA 1:1) to afford methyl 3-formylpicolinate (220 mg, 36%) as a light yellow solid. MS m/z: 166 [M+H]⁺.

Step 3: methyl 3-(2,2-difluorovinyl)picolinate: A mixture of methyl 3-formylpicolinate (220 mg, 1.33 mmol, 1.0 equiv.), sodium 2-bromo-2,2-difluoroacetate (393 mg, 2.00 mmol, 1.5 equiv.) and PPh₃ (524 mg, 2.00 mmol, 1.5 equiv.) in DMF (5 mL) was stirred for 1 h at 100° C. under air atmosphere. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by Prep-TLC (PE/EA 1:1) to afford methyl 3-(2,2-difluorovinyl)picolinate (130 mg, 49%) as a light yellow solid. MS m/z: 200 [M+H]⁺.

Step 4: methyl 3-(2,2-difluoroethyl)picolinate: Followed the General Procedure AJ using 3-(2,2-difluorovinyl)picolinate (130 mg, 0.653 mmol, 1.0 equiv.) as the starting material to give the crude product methyl 3-(2,2-difluoroethyl)picolinate (150) as a light yellow solid. MS m/z: 202 [M+H]⁺.

Step 5: (3-(2,2-difluoroethyl)pyridin-2-yl)methanol: Followed the General Procedure I using methyl 3-(2,2-difluoroethyl)picolinate (150 mg, 0.746 mmol, 1.0 equiv.) as the starting materials to give (3-(2,2-difluoroethyl)pyridin-2-yl)methanol (60 mg, 46%) as a light yellow solid. MS m/z: 174 [M+H]⁺.

Step 6: 2-(chloromethyl)-3-(2,2-difluoroethyl)pyridine: Followed the General Procedure J using (3-(2,2-difluoroethyl)pyridin-2-yl)methanol (60.0 mg, 0.346 mmol, 1.0 equiv.) as the starting material to give the crude product 2-(chloromethyl)-3-(2,2-difluoroethyl)pyridine (60 mg) as a light yellow solid. MS m/z: 192 [M+H]⁺.

Step 7: 5-((3-(2,2-difluoroethyl)pyridin-2-yl)methyl)-7-((1r,4r)-4-(2-fluoro-6-methylphenyl)cyclohexyl)-3-methylpyrido[2,3-b]pyrazin-6(5H)-one: Followed the General Procedure AP using 2-(chloromethyl)-3-(2,2-difluoroethyl)pyridine (60 mg, 0.313 mmol, 1.0 equiv.) and 7-((1r,4r)-4-(2-fluoro-6-methylphenyl)cyclohexyl)-3-methylpyrido[2,3-b]pyrazin-6(5H)-one (121 mg, 0.344 mmol, 1.1 equiv.) as the starting materials to give 5-((3-(2,2-difluoroethyl)pyridin-2-yl)methyl)-7-((1r,4r)-4-(2-fluoro-6-methylphenyl)cyclohexyl)-3-methylpyrido[2,3-b]pyrazin-6(5H)-one (56 mg, 31.9%) as a white solid. ¹H NMR (400 MHz, Chloroform-d) δ 8.35 (d, J=9.8 Hz, 2H), 7.80 (s, 1H), 7.66 (s, 1H), 7.21 (s, 1H), 7.03 (td, J=7.8, 5.4 Hz, 1H), 6.92 (d, J=7.5 Hz, 1H), 6.85 (dd, J=11.6, 8.3 Hz, 1H), 6.21 (tt, J=56.0, 4.2 Hz, 1H), 5.90 (s, 2H), 3.50 (td, J=17.2, 4.2 Hz, 2H), 3.12 (t, J=12.0 Hz, 1H), 2.90 (t, J=12.2 Hz, 1H), 2.57 (s, 3H), 2.37 (s, 3H), 2.15 (t, J=12.5 Hz, 4H), 1.88-1.79 (m, 2H), 1.52 (d, J=12.1 Hz, 2H). MS m/z: 507.15 [M+H]⁺.

### 1-((3-(Difluoromethoxy)pyrazin-2-yl)methyl)-3-((1r,4r)-4-(2-fluoro-6-methylphenyl)cyclohexyl)-7-methyl-1,8-naphthyridin-2(1H)-one (233)

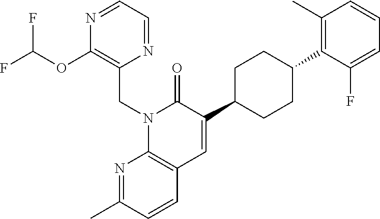 [SMILES: Cc1ccc2cc([C@H]3CC[C@H](c4c(C)cccc4F)CC3)c(=O)n(Cc3nccnc3OC(F)F)c2n1]

Step 1: 2-methyl-3-((tetrahydro-2H-pyran-2-yl)oxy)pyrazine: Followed the General Procedure AT using 3-methylpyrazin-2-ol (1 g, 9.08 mmol, 1 equiv.) and DHP (2.29 g, 27.2 mmol, 3.0 equiv.) as the starting materials to give 2-methyl-3-((tetrahydro-2H-pyran-2-yl)oxy)pyrazine (910 mg, 51%) as a colorless oil. MS m/z: 195 [M+H]⁺.

Step 2: 2-(bromomethyl)-3-((tetrahydro-2H-pyran-2-yl)oxy)pyrazine: Followed the General Procedure AF using 2-methyl-3-((tetrahydro-2H-pyran-2-yl)oxy)pyrazine (860 mg, 4.428 mmol, 1 equiv.) and NBS (1.18 g, 6.64 mmol, 1.5 equiv.) as the starting materials to give 2-(bromomethyl)-3-((tetrahydro-2H-pyran-2-yl)oxy)pyrazine (200 mg, 16%) as a light green oil. MS m/z: 273 [M+H]⁺.

Step 3: 3-((1r,4r)-4-(2-fluoro-6-methylphenyl)cyclohexyl)-7-methyl-1-((3-((tetrahydro-2H-pyran-2-yl)oxy)pyrazin-2-yl)methyl)-1,8-naphthyridin-2(1H)-one: Followed the General Procedure E using 3-((1r,4r)-4-(2-fluoro-6-methylphenyl)cyclohexyl)-7-methyl-1,8-naphthyridin-2(1H)-one (150 mg, 0.428 mmol, 1 equiv.) and 2-(bromomethyl)-3-((tetrahydro-2H-pyran-2-yl)oxy)pyrazine (175 mg, 0.642 mmol, 1.5 equiv.) as the starting materials to give 3-((1r,4r)-4-(2-fluoro-6-methylphenyl)cyclohexyl)-7-methyl-1-((3-((tetrahydro-2H-pyran-2-yl)oxy)pyrazin-2-yl)methyl)-1,8-naphthyridin-2(1H)-one (180 mg, 51%) as a white solid. MS m/z: 543 [M+H]⁺.

Step 4: 3-((1r,4r)-4-(2-fluoro-6-methylphenyl)cyclohexyl)-1-((3-hydroxypyrazin-2-yl)methyl)-7-methyl-1,8-naphthyridin-2(1H)-one: Followed the General Procedure F using 3-((1r,4r)-4-(2-fluoro-6-methylphenyl)cyclohexyl)-7-methyl-1-((3-((tetrahydro-2H-pyran-2-yl)oxy)pyrazin-2-yl)methyl)-1,8-naphthyridin-2(1H)-one 180 mg) as the starting material to give the crude product 3-((1r,4r)-4-(2-fluoro-6-methylphenyl)cyclohexyl)-1-((3-hydroxypyrazin-2-yl)methyl)-7-methyl-1,8-naphthyridin-2(1H)-one (150 mg)

Step 5: 1-((3-(difluoromethoxy)pyrazin-2-yl)methyl)-3-((1r,4r)-4-(2-fluoro-6-methylphenyl)cyclohexyl)-7-methyl-1,8-naphthyridin-2(1H)-one: Followed the General Procedure AU using 3-((1r,4r)-4-(2-fluoro-6-methylphenyl)cyclohexyl)-1-((3-hydroxypyrazin-2-yl)methyl)-7-methyl-1,8-naphthyridin-2(1H)-one (150 mg, 0.327 mmol, 1 equiv.) and methyl 2-chloro-2,2-difluoroacetate (56.7 mg, 0.392 mmol, 1.2 equiv.) as the starting materials to give 1-((3-(difluoromethoxy)pyrazin-2-yl)methyl)-3-((1r,4r)-4-(2-fluoro-6-methylphenyl)cyclohexyl)-7-methyl-1,8-naphthyridin-2(1H)-one (20.4 mg, 11.6%) as a white solid. ¹H NMR (400 MHz, Chloroform-d) δ 8.12 (d, J=2.7 Hz, 1H), 7.95 (d, J=2.7 Hz, 1H), 7.75 (d, J=7.8 Hz, 1H), 7.69-7.33 (m, 2H), 7.02 (dd, J=10.6, 6.7 Hz, 2H), 6.93-6.80 (m, 2H), 5.99 (s, 2H), 3.10 (t, J=12.0 Hz, 1H), 2.89 (t, J=12.5 Hz, 1H), 2.50 (s, 3H), 2.37 (s, 3H), 2.15 (t, J=13.5 Hz, 4H), 1.84 (d, J=12.7 Hz, 2H), 1.52 (d, J=12.6 Hz, 2H). MS m/z: 509.15 [M+H]⁺.

### 3-((1r,4r)-4-(2-Methoxy-4-methylpyridin-3-yl)cyclohexyl)-7-methyl-1-((3-(trifluoromethyl)pyridin-2-yl)methyl)-1,8-naphthyridin-2(1H)-one (234)

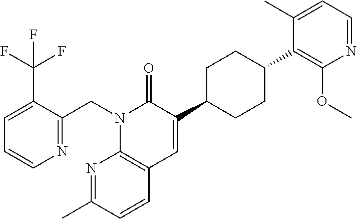 [SMILES: COc1nccc(C)c1[C@H]1CC[C@H](c2cc3ccc(C)nc3n(Cc3ncccc3C(F)(F)F)c2=O)CC1]

Step 1: 3-((1r,4r)-4-(2-methoxy-4-methylpyridin-3-yl)cyclohexyl)-7-methyl-1,8-naphthyridin-2(1H)-one: Followed the General Procedure D using ethyl 2-(4-(2-methoxy-4-methylpyridin-3-yl)cyclohexyl)acetate (350 mg, 1.20 mmol, 1 equiv) and 2-amino-6-methylnicotinaldehyde (197 mg, 1.44 mmol, 1.2 equiv) as the starting materials to give 3-((1r,4r)-4-(2-methoxy-4-methylpyridin-3-yl)cyclohexyl)-7-methyl-1,8-naphthyridin-2(1H)-one (80 mg, 18%) as a yellow solid. MS m/z: 364 [M+H]⁺.

Step 2: 3-((1r,4r)-4-(2-methoxy-4-methylpyridin-3-yl)cyclohexyl)-7-methyl-1-((3-(trifluorometh-yl) pyridine-2-yl)methyl)-1,8-naphthyridin-2(1H)-one: Followed the General Procedure AP using 3-((1r,4r)-4-(2-methoxy-4-methylpyridin-3-yl)cyclohexyl)-7-methyl-1,8-naphthyridin-2(1H)-one (80 mg, 0.22 mmol, 1 equiv) and 2-(chloromethyl)-3-(trifluoromethyl)pyridine (51.66 mg, 0.26 mmol, 1.2 equiv) as the starting materials to give 3-((1r,4r)-4-(2-methoxy-4-methylpyridin-3-yl)cyclohexyl)-7-methyl-1-((3-(trifluoromethyl)pyridine-2-yl)methyl)-1,8-naphthyridin-2(1H)-one (45.5 mg, 37.6%) as a white solid. ¹H NMR (400 MHz, Chloroform-d) δ 8.45 (d, J=4.8 Hz, 1H), 7.94 (dd, J=14.3, 6.6 Hz, 2H), 7.74 (d, J=7.8 Hz, 1H), 7.53 (s, 1H), 7.20 (dd, J=7.9, 5.2 Hz, 1H), 6.99 (d, J=7.8 Hz, 1H), 6.75 (s, 1H), 6.11 (s, 2H), 4.03 (s, 3H), 3.12 (t, J=12.1 Hz, 1H), 2.95 (s, 1H), 2.47 (s, 3H), 2.34 (d, J=29.6 Hz, 5H), 2.12 (d, J=12.5 Hz, 2H), 1.67 (d, J=13.2 Hz, 2H), 1.50-1.42 (m, 2H). MS m/z: 523.1 [M+H]⁺.

### 5-((3-Cyclopropylpyridin-2-yl)methyl)-7-((1r,4r)-4-(2,6-difluorophenyl)cyclohexyl)-3-methylpyrido[2,3-b]pyrazin-6(5H)-one (235)

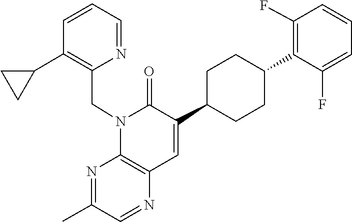 [SMILES: Cc1cnc2cc([C@H]3CC[C@H](c4c(F)cccc4F)CC3)c(=O)n(Cc3ncccc3C3CC3)c2n1]

Step 1: 5-((3-cyclopropylpyridin-2-yl)methyl)-7-((1r,4r)-4-(2,6-difluorophenyl)cyclohexyl)-3-methylpyrido[2,3-b]pyrazin-6(5H)-one: Followed the General Procedure AP using 7-((1r,4r)-4-(2,6-difluorophenyl)cyclohexyl)-3-methylpyrido[2,3-b]pyrazin-6(5H)-one (30 mg, 0.084 mmol, 1 equiv.) and 2-(chloromethyl)-3-cyclopropylpyridine (28.3 mg, 0.168 mmol, 2.0 equiv.) as the starting materials to give 5-((3-cyclopropylpyridin-2-yl)methyl)-7-((1r,4r)-4-(2,6-difluorophenyl)cyclohexyl)-3-methylpyrido[2,3-b]pyrazin-6(5H)-one (25.6 mg, 59.7%) as a white solid. ¹H NMR (400 MHz, Chloroform-d) δ 8.32 (s, 1H), 8.26-8.09 (m, 1H), 7.80 (s, 1H), 7.51-7.35 (m, 1H), 7.16-6.96 (m, 2H), 6.87-6.77 (m, 2H), 6.07 (s, 2H), 3.23-3.04 (m, 2H), 2.54 (s, 3H), 2.24-2.02 (m, 5H), 1.95-1.82 (m, 2H), 1.55-1.48 (m, 2H), 1.13-1.04 (m, 2H), 0.85-0.79 (m, 2H). MS m/z: 487.25[M+H]⁺.

### 7-((1r,4r)-4-(2,6-Difluorophenyl)cyclohexyl)-3-methyl-5-((3-(2,2,2-trifluoroethyl)pyridin-2-yl)methyl)pyrido[2,3-b]pyrazin-6(5H)-one (236)

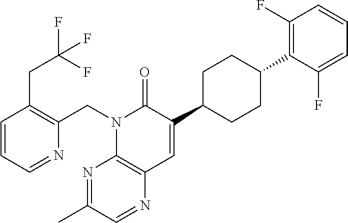 [SMILES: Cc1cnc2cc([C@H]3CC[C@H](c4c(F)cccc4F)CC3)c(=O)n(Cc3ncccc3CC(F)(F)F)c2n1]

Step 1: 7-((1r,4r)-4-(2,6-difluorophenyl)cyclohexyl)-3-methyl-5-((3-(2,2,2-trifluoroethyl)pyridin-2-yl)methyl)pyrido[2,3-b]pyrazin-6(5H)-one: Followed the General Procedure AP using 2-(chloromethyl)-3-(2,2,2-trifluoroethyl)pyridine (35.0 mg, 0.167 mmol, 1.0 equiv.) and 7-((1r,4r)-4-(2,6-difluorophenyl)cyclohexyl)-3-methylpyrido[2,3-b]pyrazin-6(5H)-one (44.7 mg, 0.313 mmol, 1.2 equiv.) as the starting materials to give 7-((1r,4r)-4-(2,6-difluorophenyl)cyclohexyl)-3-methyl-5-((3-(2,2,2-trifluoroethyl)pyridin-2-yl)methyl)pyrido[2,3-b]pyrazin-6(5H)-one (35.7 mg, 37.4%) as a white solid. ¹H NMR (400 MHz, Chloroform-d) δ 8.43-8.33 (m, 2H), 7.79 (d, J=0.9 Hz, 1H), 7.67 (d, J=7.7 Hz, 1H), 7.20 (t, J=6.4 Hz, 1H), 7.11 (tt, J=8.4, 6.3 Hz, 1H), 6.82 (t, J=8.6 Hz, 2H), 5.89 (s, 2H), 3.81 (q, J=10.5 Hz, 2H), 3.10 (t, J=12.1 Hz, 2H), 2.58 (s, 3H), 2.11 (d, J=12.2 Hz, 4H), 1.94-1.84 (m, 2H), 1.56-1.49 (m, 2H). MS m/z: 529.2 [M+H]⁺

### 7-((1r,4r)-4-(3-Fluoro-5-methylpyridin-4-yl)cyclohexyl)-3-methyl-5-((3-(trifluoromethyl)pyridin-2-yl)methyl)pyrido[2,3-b]pyrazin-6(5H)-one (237)

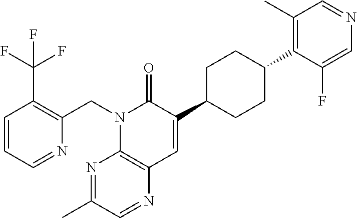 [SMILES: Cc1cnc2cc([C@H]3CC[C@H](c4c(C)cncc4F)CC3)c(=O)n(Cc3ncccc3C(F)(F)F)c2n1]

Step 1: ethyl 2-(4-(3-chloro-5-fluoropyridin-4-yl)cyclohex-3-en-1-yl)acetate: Followed the General Procedure AL using 4-bromo-3-chloro-5-fluoropyridine (1 g, 4.75 mmol, 1 equiv) and ethyl 2-(4-(4,4,5,5-tetramethyl-1,3,2-dioxaborolan-2-yl)cyclohex-3-en-1-yl)acetate (1.4 g, 4.75 mmol, 1 equiv) as the starting materials to give ethyl 2-(4-(3-chloro-5-fluoropyridin-4-yl)cyclohex-3-en-1-yl)acetate (800 mg, 52%) as a white solid. MS m/z: 298 [M+H]⁺.

Step 2: ethyl 2-(4-(3-fluoro-5-methylpyridin-4-yl)cyclohex-3-en-1-yl)acetate: Followed the General Procedure AL using ethyl 2-(4-(3-chloro-5-fluoropyridin-4-yl)cyclohex-3-en-1-yl)acetate (800 mg, 2.68 mmol, 1 equiv) and methylboronic acid (209 mg, 3.49 mmol, 1.3 equiv) as the starting materials to give ethyl 2-(4-(3-fluoro-5-methylpyridin-4-yl)cyclohex-3-en-1-yl)acetate (450 mg, 57%) as a white solid. MS m/z: 278 [M+H]⁺.

Step 3: ethyl 2-(4-(3-fluoro-5-methylpyridin-4-yl)cyclohexyl)acetate: Followed the General Procedure AR using ethyl 2-(4-(3-fluoro-5-methylpyridin-4-yl)cyclohex-3-en-1-yl)acetate (450 mg, 1.62 mmol, 1 equiv) as the starting material to give the crude product ethyl 2-(4-(3-fluoro-5-methylpyridin-4-yl)cyclohexyl)acetate (270 mg) as a colorless oil. MS m/z: 280 [M+H]⁺.

Step 4: 7-((1r,4r)-4-(3-fluoro-5-methylpyridin-4-yl)cyclohexyl)-3-methylpyrido[2,3-b]pyrazin-6(5H)-one: Followed the General Procedure D using ethyl 2-(4-(3-fluoro-5-methylpyridin-4-yl)cyclohexyl)acetate (270 mg, 0.758 mmol, 1 equiv) and 3-amino-5-methylpyrazine-2-carbaldehyde (124 mg, 0.909 mmol, 1.2 equiv) as the starting materials to give 7-((1r,4r)-4-(3-fluoro-5-methylpyridin-4-yl)cyclohexyl)-3-methylpyrido[2,3-b]pyrazin-6(5H)-one (100 mg, 34%) as a white solid. MS m/z: 353 [M+H]⁺.

Step 5: 7-((1r,4r)-4-(3-fluoro-5-methylpyridin-4-yl)cyclohexyl)-3-methyl-5-((3-(trifluoromethyl)pyridin-2-yl)methyl)pyrido[2,3-b]pyrazin-6(5H)-one: Followed the General Procedure AP using 7-((1r,4r)-4-(3-fluoro-5-methylpyridin-4-yl)cyclohexyl)-3-methylpyrido[2,3-b]pyrazin-6(5H)-one (40 mg, 0.114 mmol, 1 equiv) and 2-(chloromethyl)-3-(trifluoromethyl)pyridine (29 mg, 0.148 mmol, 1.3 equiv) as the starting materials to give 7-((1r,4r)-4-(3-fluoro-5-methylpyridin-4-yl)cyclohexyl)-3-methyl-5-((3-(trifluoromethyl)pyridin-2-yl)methyl)pyrido[2,3-b]pyrazin-6(5H)-one (9 mg, 14.70%) as a white solid. ¹H NMR (400 MHz, Chloroform-d) δ 8.47-8.39 (m, 1H), 8.33 (s, 1H), 8.21 (s, 2H), 8.02-7.95 (m, 1H), 7.80 (s, 1H), 7.24 (dd, J=7.9, 4.9 Hz, 1H), 6.03 (s, 2H), 3.21-3.11 (m, 1H), 2.92 (t, J=12.5 Hz, 1H), 2.52 (s, 3H), 2.39 (s, 3H), 2.16 (t, J=16.5 Hz, 4H), 1.87 (d, J=12.6 Hz, 2H), 1.63-1.49 (m, 2H). MS m/z: 512.05 [M+H]⁺.

### 5-((3-Cyclopropoxypyridin-2-yl)methyl)-7-((1r,4r)-4-(2-fluoro-6-methylphenyl)cyclohexyl)-3-methylpyrido[2,3-b]pyrazin-6(5H)-one (238)

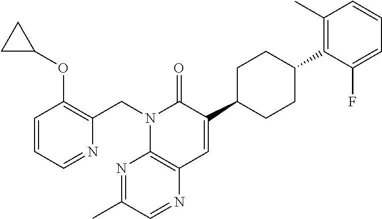 [SMILES: Cc1cnc2cc([C@H]3CC[C@H](c4c(C)cccc4F)CC3)c(=O)n(Cc3ncccc3OC3CC3)c2n1]

Step 1: ethyl 3-cyclopropoxypicolinate: Followed the General Procedure H using 2-bromo-3-cyclopropoxypyridine (300 mg, 1.40 mmol, 1 equiv) as the starting material to give ethyl 3-cyclopropoxypicolinate (200 mg, 68%) as a yellow oil. MS m/z: 208 [M+H]⁺.

Step 2: (3-cyclopropoxypyridin-2-yl)methanol: Followed the General Procedure I using ethyl 3-cyclopropoxypicolinate (180 mg, 0.867 mmol, 1 equiv) as the starting material to give (3-cyclopropoxypyridin-2-yl)methanol (110 mg, 76%) as a yellow oil. MS m/z: 166 [M+H]⁺.

Step 3: 2-(chloromethyl)-3-cyclopropoxypyridine: Followed the General Procedure J using (3-cyclopropoxypyridin-2-yl)methanol (110 mg, 0.67 mmol, 1 equiv) as the starting materials to give the crude product 2-(chloromethyl)-3-cyclopropoxypyridine (100 mg) as a yellow oil. MS m/z: 184 [M+H]⁺.

Step 4: 5-((3-cyclopropoxypyridin-2-yl)methyl)-7-((1r,4r)-4-(2-fluoro-6-methylphenyl)cyclohexyl)-3-methylpyrido[2,3-b]pyrazin-6(5H)-one: Followed the General Procedure AP using 2-(chloromethyl)-3-cyclopropoxypyridine (80 mg, 0.43 mmol, 1 equiv) and 7-((1r,4r)-4-(2-fluoro-6-methylphenyl)cyclohexyl)-3-methylpyrido[2,3-b]pyrazin-6(5H)-one (151 mg, 0.43 mmol, 1 equiv) as the starting materials to give 5-((3-cyclopropoxypyridin-2-yl)methyl)-7-((1r,4r)-4-(2-fluoro-6-methylphenyl)cyclohexyl)-3-methylpyrido[2,3-b]pyrazin-6(5H)-one (44.5 mg, 15.7%) as a white solid. ¹H NMR (400 MHz, Chloroform-d) δ 8.31 (s, 1H), 7.98 (s, 1H), 7.78 (d, J=0.9 Hz, 1H), 7.56 (s, 1H), 7.15 (s, 1H), 7.03 (td, J=7.9, 5.5 Hz, 1H), 6.91 (d, J=7.5 Hz, 1H), 6.84 (dd, J=11.8, 8.1 Hz, 1H), 5.84 (s, 2H), 3.89-3.80 (m, 1H), 3.13 (t, J=11.9 Hz, 1H), 2.90 (t, J=12.1 Hz, 1H), 2.53 (s, 3H), 2.37 (s, 3H), 2.15 (d, J=12.2 Hz, 4H), 1.89-1.80 (s, 2H), 1.84 (d, J=11.7 Hz, 2H), 0.89-0.80 (m, 4H). MS m/z: 499.3 [M+H]⁺.

### 7-((1r,4r)-4-(2,6-Difluorophenyl)cyclohexyl)-3-methyl-5-((3-(2,2,2-trifluoroethyl)pyridin-2-yl)methyl)pyrido[2,3-b]pyrazin-6(5H)-one (239)

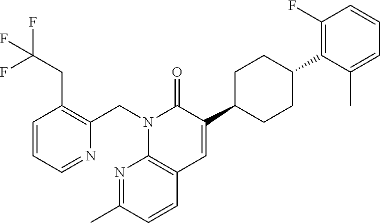 [SMILES: Cc1ccc2cc([C@H]3CC[C@H](c4c(C)cccc4F)CC3)c(=O)n(Cc3ncccc3CC(F)(F)F)c2n1]

Step 1: 3-((1r,4r)-4-(2-fluoro-6-methylphenyl)cyclohexyl)-7-methyl-1-((3-(2,2,2-trifluoroethyl)pyridin-2-yl)methyl)-1,8-naphthyridin-2(1H)-one: Followed the General Procedure AP using 3-((1r,4r)-4-(2-fluoro-6-methylphenyl)cyclohexyl)-7-methyl-1,8-naphthyridin-2(1H)-one (55.0 mg, 0.157 mmol, 1.0 equiv.) and 2-(chloromethyl)-3-(2,2,2-trifluoroethyl)pyridine (39.5 mg, 0.188 mmol, 1.2 equiv.) as the starting materials to give 3-((1r,4r)-4-(2-fluoro-6-methylphenyl)cyclohexyl)-7-methyl-1-((3-(2,2,2-trifluoroethyl)pyridin-2-yl)methyl)-1,8-naphthyridin-2(1H)-one (32.2 mg, 39.2%) as a white solid. ¹H NMR (400 MHz, Chloroform-d) δ 8.51 (s, 1H), 7.74 (d, J=7.8 Hz, 2H), 7.52 (s, 1H), 7.34 (s, 1H), 7.08-6.97 (m, 2H), 6.91 (d, J=7.4 Hz, 1H), 6.85 (dd, J=11.8, 8.1 Hz, 1H), 6.02 (s, 2H), 3.84 (q, J=10.5 Hz, 2H), 3.10 (t, J=12.0 Hz, 1H), 2.89 (t, J=12.3 Hz, 1H), 2.54 (s, 3H), 2.37 (s, 3H), 2.14 (dd, J=19.3, 13.4 Hz, 4H), 1.83 (d, J=12.7 Hz, 2H), 1.50 (d, J=12.3 Hz, 2H). MS m/z: 524.20 [M+H]⁺.

### 3-(Difluoromethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (240)

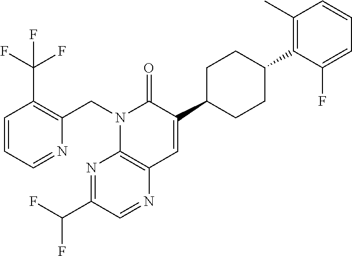 [SMILES: Cc1cccc(F)c1[C@H]1CC[C@H](c2cc3ncc(C(F)F)nc3n(Cc3ncccc3C(F)(F)F)c2=O)CC1]

Step 1: 7-((1r,4r)-4-(2-fluoro-6-methylphenyl)cyclohexyl)-3-(hydroxymethyl)-5-((3-(trifluoromethyl)pyridin-2-yl)methyl)pyrido[2,3-b]pyrazin-6(5H)-one: Followed the General Procedure V using 3-chloro-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (100 mg, 0.188 mmol, 1 equiv) as the starting material to give 7-((1r,4r)-4-(2-fluoro-6-methylphenyl)cyclohexyl)-3-(hydroxymethyl)-5-((3-(trifluoromethyl)pyridin-2-yl)methyl)pyrido[2,3-b]pyrazin-6(5H)-one (80 mg, 80%) as a white solid. MS m/z: 527 [M+H]⁺.

Step 2: 7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazine-3-carbaldehyde: Followed the General Procedure AM using 7-((1r,4r)-4-(2-fluoro-6-methylphenyl)cyclohexyl)-3-(hydroxymethyl)-5-((3-(trifluoromethyl)pyridin-2-yl)methyl)pyrido[2,3-b]pyrazin-6(5H)-one (80 mg, 0.152 mmol, 1 equiv) and manganese dioxide (26.42 mg, 0.304 mmol, 2 equiv) as the starting materials to give the crude product 7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazine-3-carbaldehyde (50 mg) as a white solid. MS m/z: 525 [M+H]⁺.

Step 3: 3-(difluoromethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the General Procedure AE using 7-((1r,4r)-4-(2-fluoro-6-methylphenyl)cyclohexyl)-6-oxo-5-((3-(trifluoromethyl)pyridin-2-yl)methyl)-5,6-dihydropyrido[2,3-b]pyrazine-3-carbaldehyde (50 mg, 0.104 mmol, 1 equiv) as the starting material to give 3-(difluoromethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (22.6 mg, 37.8%) as a white solid. ¹H NMR (400 MHz, Chloroform-d) δ 8.76 (s, 1H), 8.42 (d, J=4.6 Hz, 1H), 8.00 (d, J=7.7 Hz, 1H), 7.88 (d, J=0.9 Hz, 1H), 7.28 (s, 1H), 7.09-6.99 (m, 1H), 6.92 (d, J=7.5 Hz, 1H), 6.90-6.80 (m, 1H), 6.55 (t, J=54.7 Hz, 1H), 6.01 (s, 2H), 3.18 (t, J=12.1 Hz, 1H), 2.92 (t, J=12.4 Hz, 1H), 2.38 (s, 3H), 2.18 (d, J=12.1 Hz, 4H), 1.87 (d, J=10.5 Hz, 2H), 1.57 (t, J=12.3 Hz, 2H). MS m/z: 547.05 [M+H]⁺.

### 1-((3-Cyclopropylpyridin-2-yl)methyl)-3-((1r,4r)-4-(2-fluoro-6-methylphenyl)cyclohexyl)-7-methyl-1,8-naphthyridin-2(1H)-one (241)

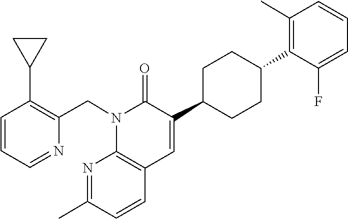 [SMILES: Cc1ccc2cc([C@H]3CC[C@H](c4c(C)cccc4F)CC3)c(=O)n(Cc3ncccc3C3CC3)c2n1]

Step 1: 1-((3-cyclopropylpyridin-2-yl)methyl)-3-((1r,4r)-4-(2-fluoro-6-methylphenyl)cyclohexyl)-7-methyl-1,8-naphthyridin-2(1H)-one: Followed the General Procedure AP using 2-(chloromethyl)-3-cyclopropylpyridine (30 mg, 0.179 mmol, 1 equiv.) and 3-((1r,4r)-4-(2-fluoro-6-methylphenyl)cyclohexyl)-7-methyl-1,8-naphthyridin-2(1H)-one (75.2 mg, 0.215 mmol, 1.2 equiv.) as the starting materials to give 1-((3-cyclopropylpyridin-2-yl)methyl)-3-((1r,4r)-4-(2-fluoro-6-methylphenyl)cyclohexyl)-7-methyl-1,8-naphthyridin-2(1H)-one (12.5 mg, 14.1%) as a white solid. ¹H NMR (400 MHz, Chloroform-d) δ 8.29 (s, 1H), 7.74 (d, J=7.8 Hz, 1H), 7.54 (s, 2H), 7.20-6.79 (m, 5H), 6.19 (s, 2H), 3.13 (t, J=12.0 Hz, 1H), 2.90 (t, J=12.3 Hz, 1H), 2.50 (s, 3H), 2.37 (s, 3H), 2.30-2.07 (m, 5H), 1.83 (d, J=11.2 Hz, 2H), 1.47 (d, J=31.1 Hz, 2H), 1.09-1.00 (m, 2H), 0.80 (t, J=5.3 Hz, 2H). MS m/z: 482.30 [M+H]⁺.

### 3-Methyl-7-((1r,4r)-4-(5-methylisothiazol-4-yl)cyclohexyl)-5-((3-(trifluoromethyl)pyrazin-2-yl)methyl)pyrido[2,3-b]pyrazin-6(5H)-one (242)

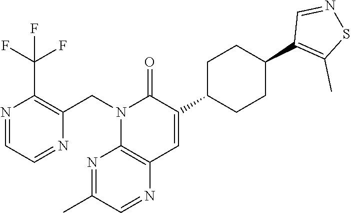 [SMILES: Cc1cnc2cc([C@H]3CC[C@H](c4cnsc4C)CC3)c(=O)n(Cc3nccnc3C(F)(F)F)c2n1]

Step 1: ethyl 2-(4-(5-methylisothiazol-4-yl)cyclohex-3-en-1-yl)acetate: Followed the General Procedure AL using ethyl 2-(4-(4,4,5,5-tetramethyl-1,3,2-dioxaborolan-2-yl)cyclohex-3-en-1-yl)acetate (250 mg, 0.847 mmol, 1 equiv.) and 4-bromo-5-methylisothiazole (150 mg, 0.847 mmol, 1 equiv.) as the starting materials to give ethyl 2-(4-(5-methylisothiazol-4-yl)cyclohex-3-en-1-yl)acetate (100 mg, 44%) as a light yellow oil. MS m/z: 266 [M+H]⁺.

Step 2: ethyl 2-(4-(5-methylisothiazol-4-yl)cyclohexyl)acetate: Followed the General Procedure AR using ethyl 2-(4-(5-methylisothiazol-4-yl)cyclohex-3-en-1-yl)acetate (100 mg, 0.377 mmol, 1.0 equiv.) as the starting material to give the crude product ethyl 2-(4-(5-methylisothiazol-4-yl)cyclohexyl)acetate (80 mg) as a light yellow oil. MS m/z: 268 [M+H]⁺.

Step 3: 3-methyl-7-((1r,4r)-4-(5-methylisothiazol-4-yl)cyclohexyl)pyrido[2,3-b]pyrazin-6(5H)-one: Followed the General Procedure D using ethyl 2-(4-(5-methylisothiazol-4-yl)cyclohexyl)acetate (80 mg, 0.299 mmol, 1.0 equiv.) and 3-amino-5-methylpyrazine-2-carbaldehyde (49 mg, 0.359 mmol, 1.2 equiv.) as the starting materials to give 3-methyl-7-((1r,4r)-4-(5-methylisothiazol-4-yl)cyclohexyl)pyrido[2,3-b]pyrazin-6(5H)-one (50 mg, 49%). MS m/z: 341 [M+H]⁺.

Step 4: 3-methyl-7-((1r,4r)-4-(5-methylisothiazol-4-yl)cyclohexyl)-5-((3-(trifluoromethyl)pyrazin-2-yl)methyl)pyrido[2,3-b]pyrazin-6(5H)-one: Followed the General Procedure AP using 3-methyl-7-((1r,4r)-4-(5-methylisothiazol-4-yl)cyclohexyl)pyrido[2,3-b]pyrazin-6(5H)-one (50 mg, 0.147 mmol, 1 equiv.) and 2-(bromomethyl)-3-(trifluoromethyl)pyrazine hydrochloride (60.8 mg, 0.22 mmol, 1.5 equiv.) as the starting materials to give 3-methyl-7-((1r,4r)-4-(5-methylisothiazol-4-yl)cyclohexyl)-5-((3-(trifluoromethyl)pyrazin-2-yl)methyl)pyrido[2,3-b]pyrazin-6(5H)-one (9.1 mg, 12.3%) as a white solid. ¹H NMR (400 MHz, Chloroform-d) δ 8.51 (d, J=2.3 Hz, 1H), 8.46 (d, J=2.3 Hz, 1H), 8.38-8.27 (m, 2H), 7.85 (s, 1H), 6.08 (s, 2H), 3.09 (tt, J=11.8, 3.1 Hz, 1H), 2.71 (tt, J=12.2, 3.7 Hz, 1H), 2.52 (s, 311), 2.49 (s, 311), 2.21-2.14 (m, 2H), 2.05-1.95 (i, 2H), 1.78-1.67 (m, 2H), 1.66-1.52 (m, 2H). MS m/z: 501.0 [M+H]⁺.

### 3-((1r,3r)-3-(2-Fluoro-6-methylphenyl)cyclobutyl)-7-methyl-1-((3-(trifluoromethyl)pyridin-2-yl)methyl)-1,8-naphthyridin-2(1H)-one (243A); 3-((1s,3s)-3-(2-Fluoro-6-methylphenyl)cyclobutyl)-7-methyl-1-((3-(trifluoromethyl)pyridin-2-yl)methyl)-1,8-naphthyridin-2(1H)-one (243B)

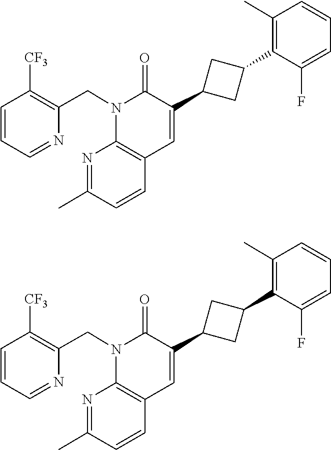 [SMILES: Cc1ccc2cc([C@H]3C[C@H](c4c(C)cccc4F)C3)c(=O)n(Cc3ncccc3C(F)(F)F)c2n1 ; Cc1ccc2cc([C@H]3C[C@@H](c4c(C)cccc4F)C3)c(=O)n(Cc3ncccc3C(F)(F)F)c2n1]

Step 1: 1-fluoro-3-methyl-2-vinylbenzene: To a stirred mixture of methyltriphenylphosphanium bromide (3.88 g, 10.8 mmol, 1.5 equiv) in THF (10 mL) was added t-BuOK (1.22 g, 10.8 mmol, 1.5 equiv.) at room temperature. The resulting mixture was stirred for 10 min at room temperature. To the above mixture was added 2-fluoro-6-methylbenzaldehyde (1 g, 7.24 mmol, 1 equiv.) in THF (3 mL) dropwise at room temperature. The resulting mixture was stirred for overnight at room temperature under argon atmosphere. The residue was purified by silica gel column chromatography, eluted with PE/EA (0% to 100% gradient in 15 min) to afford 1-fluoro-3-methyl-2-vinylbenzene (600 mg, 60%) as a colorless oil. MS m/z: 137 [M+H]⁺.

Step 2: 2,2-dichloro-3-(2-fluoro-6-methylphenyl)cyclobutan-1-one: To a stirred mixture of 1-fluoro-3-methyl-2-vinylbenzene (1 g, 7.34 mmol, 1 equiv) and Zn—Cu (2.37 g, 18.3 mmol, 2.5 equiv) in Et₂O (10 mL) at room temperature. The resulting mixture was stirred for 10 min at room temperature under air atmosphere. To the above mixture was added trichloroacetyl chloride (2.67 g, 14.6 mmol, 2 equiv) and POCl₃ (2.25 g, 14.6 mmol, 2 equiv) in Et₂O (10 mL) dropwise at room temperature under air atmosphere. The resulting mixture was stirred for overnight at room temperature. The resulting mixture was filtered, the filter cake was washed with EtOAc (3×20 mL). The filtrate was concentrated under reduced pressure. The crude product was used in the next step directly without further purification. MS m/z: 247 [M+H]⁺.

Step 3: 3-(2-fluoro-6-methylphenyl)cyclobutan-1-one: To a stirred mixture of zinc (635 mg, 9.71 mmol, 4 equiv.) in AcOH (10 mL) at room temperature under air atmosphere. The resulting mixture was stirred for 10 min at room temperature. To the above mixture was added 2,2-dichloro-3-(2-fluoro-6-methylphenyl)cyclobutan-1-one (600 mg, 2.42 mmol, 1 equiv.) in AcOH (10 mL) dropwise at room temperature. The resulting mixture was stirred for 2 h at 60° C. under nitrogen atmosphere. The resulting mixture was concentrated under reduced pressure. The residue was purified by silica gel column chromatography, eluted with PE/EA (0% to 100% gradient in 20 min) to afford 3-(2-fluoro-6-methylphenyl)cyclobutan-1-one (236 mg, 54%) as a yellow oil. MS m/z: 179 [M+H]⁺.

Step 4: ethyl 2-(3-(2-fluoro-6-methylphenyl)cyclobutylidene)acetate: To a stirred mixture of triethyl phosphonoacetate (377 mg, 1.68 mmol, 1.2 equiv) in THF (5 mL) were added NaH (84.1 mg, 2.1 mmol, 1.5 equiv., 60% in oil) in portions at 0° C. The resulting mixture was stirred for 15 min at room temperature. To the above mixture was added 3-(2-fluoro-6-methylphenyl)cyclobutan-1-one (250 mg, 1.4 mmol, 1 equiv.) in THF (5 mL) dropwise at room temperature. The resulting mixture was stirred for 2 h at room temperature. The reaction was quenched by the addition of sat. NH₄Cl (aq.) (5 mL) at 0° C. The resulting mixture was extracted with EtOAc (3×30 mL). The combined organic layers were washed with brine (1×30 mL), dried over anhydrous Na₂SO₄. After filtration, the filtrate was concentrated under reduced pressure. The residue was purified by Prep-TLC (PE/EA 25:1) to afford ethyl 2-(3-(2-fluoro-6-methylphenyl)cyclobutylidene)acetate (260 mg, 74%) as a yellow oil. MS m/z: 249 [M+H]⁺.

Step 5: ethyl 2-(3-(2-fluoro-6-methylphenyl)cyclobutyl)acetate: Followed the General Procedure K using ethyl 2-(3-(2-fluoro-6-methylphenyl)cyclobutylidene)acetate (200 mg, 0.806 mmol, 1 equiv.) as the starting material to give the crude product ethyl 2-(3-(2-fluoro-6-methylphenyl)cyclobutyl)acetate (180 mg) as a colorless oil. MS n/z: 251 [M+H]⁺.

Step 6: 3-(3-(2-fluoro-6-methylphenyl)cyclobutyl)-7-methyl-1,8-naphthyridin-2(1H)-one: Followed the General Procedure D using ethyl 2-(3-(2-fluoro-6-methylphenyl)cyclobutyl)acetate (180 mg, 0.72 mmol, 1.0 equiv.) and 2-amino-6-methylnicotinaldehyde (117 mg, 0.864 mmol, 1.2 equiv.) as the starting materials to give 3-(3-(2-fluoro-6-methylphenyl)cyclobutyl)-7-methyl-1,8-naphthyridin-2(1H)-one (100 mg, 43%) as a light yellow solid. MS m/z: 323 [M+H]⁺.

Step 7: 3-(3-(2-fluoro-6-methylphenyl)cyclobutyl)-7-methyl-1-((3-(trifluoromethyl)pyridin-2-yl)methyl)-1,8-naphthyridin-2(1H)-one: Followed the General Procedure AP using 3-(3-(2-fluoro-6-methylphenyl)cyclobutyl)-7-methyl-1,8-naphthyridin-2(1H)-one (100 mg, 0.31 mmol, 1 equiv.) and 2-(chloromethyl)-3-(trifluoromethyl)pyridine hydrochloride (108 mg, 0.465 mmol, 1.5 equiv.) as the starting materials to give the crude product 3-(3-(2-fluoro-6-methylphenyl)cyclobutyl)-7-methyl-1-((3-(trifluoromethyl)pyridin-2-yl)methyl)-1,8-naphthyridin-2(1H)-one (100 mg) as a white solid. The crude product (100 mg) was purified by Chiral-HPLC with the following conditions (Column: (S, S)-Whelk-O 1 5 μm Kromasil, 3*25 cm, 5 m; Mobile Phase A: Hex (10 mM NH₃-MeOH), Mobile Phase B: EtOH-HPLC; Flow rate: 40 mL/min; Gradient: isocratic 20; Wave Length: 223/326 nm; RT1 (min): 9.99; RT2 (min): 12.6; Sample Solvent: EtOH:DCM=1:1-HPLC; Injection Volume: 0.5 mL; Number of Runs: 7) to afford 3-((1r,3r)-3-(2-fluoro-6-methylphenyl)cyclobutyl)-7-methyl-1-((3-(trifluoromethyl)pyridin-2-yl)methyl)-1,8-naphthyridin-2(11)-one (53.5 mg, 53.5%) as a white solid and 3-((1s,3s)-3-(2-fluoro-6-methylphenyl)cyclobutyl)-7-methyl-1-((3-(trifluoromethyl)pyridin-2-yl)methyl)-1,8-naphthyridin-2(1H)-one (9.2 mg, 9.2%) as a white solid.

243A: ¹H NMR (400 MHz, DMSO-d₆) δ 8.51 (d, J=4.5 Hz, 1H), 8.20 (d, J=7.6 Hz, 1H), 8.07 (d, J=7.8 Hz, 1H), 7.86 (s, 1H), 7.50-7.30 (m, 1H), 7.19-7.03 (m, 2H), 7.00-6.87 (m, 2H), 5.90 (s, 2H), 3.75-3.62 (m, 1H), 3.59-3.45 (m, 1H), 2.84-2.72 (m, 2H), 2.48-2.39 (m, 2H), 2.37 (s, 3H), 2.31 (s, 3H). MS m/z: 482.2 [M+H]⁺.

243B: ¹H NMR (400 MHz, DMSO-d₆) δ 8.51 (d, J=4.8 Hz, 1H), 8.21 (d, J=7.9 Hz, 1H), 8.16 (s, 1H), 8.13 (d, J=7.8 Hz, 1H), 7.45 (dd, J=7.9, 4.9 Hz, 1H), 7.18 (d, J=7.8 Hz, 1H), 7.15-7.08 (m, 1H), 7.03-6.92 (m, 2H), 5.93 (s, 2H), 3.96-3.81 (m, 1H), 3.76-3.61 (m, 1H), 2.84-2.72 (m, 2H), 2.61-2.52 (m, 2H), 2.41 (s, 3H), 2.27 (s, 3H). MS m/z: 482.2 [M+H]⁺.

### 7-Methyl-1-((3-(trifluoromethoxy)pyridin-2-yl)methyl)-3-((1r,4r)-4-(2-(trifluoromethyl)phenyl)cyclohexyl)-1,8-naphthyridin-2(1H)-one (244)

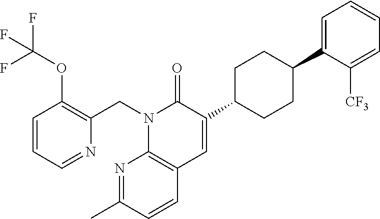 [SMILES: Cc1ccc2cc([C@H]3CC[C@H](c4ccccc4C(F)(F)F)CC3)c(=O)n(Cc3ncccc3OC(F)(F)F)c2n1]

Step 1: 7-methyl-1-((3-(trifluoromethoxy)pyridin-2-yl)methyl)-3-((1r,4r)-4-(2-(trifluoromethyl)phenyl)cyclohexyl)-1,8-naphthyridin-2(1H)-one: Followed the General Procedure AP using 7-methyl-3-((1r,4r)-4-(2-(trifluoromethyl)phenyl)cyclohexyl)-1,8-naphthyridin-2(1H)-one (50 mg, 0.129 mmol, 1 equiv) and 2-(chloromethyl)-3-(trifluoromethoxy)pyridine (32.8 mg, 0.155 mmol, 1.2 equiv.) as the starting materials to give 7-methyl-1-((3-(trifluoromethoxy)pyridin-2-yl)methyl)-3-((1r,4r)-4-(2-(trifluoromethyl)phenyl)cyclohexyl)-1,8-naphthyridin-2(1H)-one (28.1 mg, 38.4%) as a white solid. ¹H NMR (300 MHz, Chloroform-d) δ 8.35-8.26 (m, 1H), 7.75 (d, J=7.8 Hz, 1H), 7.63-7.45 (m, 5H), 7.29 (s, 1H), 7.22-7.15 (m, 1H), 7.00 (d, J=7.8 Hz, 1H), 6.04 (s, 2H), 3.21-2.97 (m, 2H), 2.49 (s, 3H), 2.15 (d, J=12.4 Hz, 2H), 1.96 (s, 2H), 1.76-1.66 (m, 2H), 1.64-1.46 (m, 2H). MS m/z: 562.1 [M+H]⁺.

### 1-((3-Cyclopropylpyridin-2-yl)methyl)-3-((1r,4r)-4-(2,6-difluorophenyl)cyclohexyl)-7-methyl-1,8-naphthyridin-2(1H)-one (245)

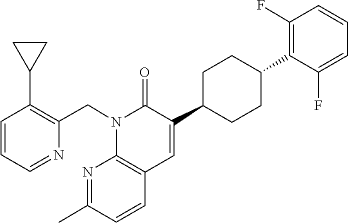 [SMILES: Cc1ccc2cc([C@H]3CC[C@H](c4c(F)cccc4F)CC3)c(=O)n(Cc3ncccc3C3CC3)c2n1]

Step 1: 1-((3-cyclopropylpyridin-2-yl)methyl)-3-((1r,4r)-4-(2,6-difluorophenyl)cyclohexyl)-7-methyl-1,8-naphthyridin-2(1H)-one: Followed the General Procedure AP using 3-((1r,4r)-4-(2,6-difluorophenyl)cyclohexyl)-7-methyl-1,8-naphthyridin-2(1H)-one (50 mg, 0.141 mmol, 1 equiv.) and 2-(chloromethyl)-3-cyclopropylpyridine (30 mg, 0.141 mmol, 1 equiv.) as the starting materials to give 3-((1r,4r)-4-(2,6-difluorophenyl)cyclohexyl)-7-methyl-1-((3-(trifluoromethyl)pyridin-2-yl)methyl)-1,8-naphthyridin-2(1H)-one (7.6 mg, 10.6%) as a white solid. ¹H NMR (400 MHz, Chloroform-d) δ 8.20 (s, 1H), 7.74 (d, J=7.8 Hz, 1H), 7.52 (s, 1H), 7.41 (s, 1H), 7.16-6.93 (m, 3H), 6.82 (t, J=8.6 Hz, 2H), 6.14 (s, 2H), 3.19-3.04 (m, 2H), 2.49 (s, 3H), 2.25-2.04 (m, 5H), 1.92-1.84 (m, 2H), 1.54-1.47 (m, 2H), 1.10-1.00 (m, 2H), 0.85-0.76 (m, 2H). MS m/z: 486.25 [M+H]⁺.

### 3-((1r,4r)-4-(2,6-Difluorophenyl)cyclohexyl)-7-methyl-1-((3-(trifluoromethoxy)pyridin-2-yl)methyl)-1,8-naphthyridin-2(1H)-one (246)

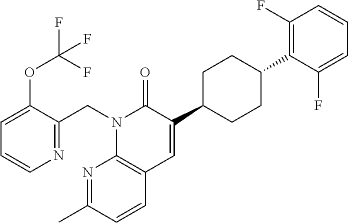 [SMILES: Cc1ccc2cc([C@H]3CC[C@H](c4c(F)cccc4F)CC3)c(=O)n(Cc3ncccc3OC(F)(F)F)c2n1]

Step 1: 3-((1r,4r)-4-(2,6-difluorophenyl)cyclohexyl)-7-methyl-1-((3-(trifluoromethoxy)pyridin-2-yl)methyl)-1,8-naphthyridin-2(1H)-one: Followed the General Procedure AP using 3-((1r,4r)-4-(2,6-difluorophenyl)cyclohexyl)-7-methyl-1,8-naphthyridin-2(1H)-one (50 mg, 0.141 mmol, 1 equiv.) and 2-(chloromethyl)-3-(trifluoromethoxy)pyridine (29.85 mg, 0.141 mmol, 1 equiv.) as the starting materials to give the product 3-((1r,4r)-4-(2,6-difluorophenyl)cyclohexyl)-7-methyl-1-((3-(trifluoromethyl)pyridin-2-yl)methyl)-1,8-naphthyridin-2(1H)-one (17.8 mg, 23.3%) as a white solid. ¹H NMR (400 MHz, Chloroform-d) δ 8.34-8.28 (m, 1H), 7.74 (d, J=7.8 Hz, 1H), 7.63-7.56 (m, 1H), 7.54-7.49 (m, 1H), 7.20 (d, J=8.3, 4.7 Hz, 1H), 7.16-7.04 (m, 1H), 6.99 (d, J=7.8 Hz, 1H), 6.82 (t, J=8.6 Hz, 2H), 6.04 (s, 2H), 3.17-3.04 (m, 2H), 2.49 (s, 3H), 2.12 (t, J=10.3 Hz, 4H), 1.92-1.83 (m, 2H), 1.58-1.42 (m, 2H). MS m/z: 530.25 [M+H]⁺.

### 7-Methyl-1-((3-(trifluoromethoxy)pyridin-2-yl)methyl)-3-((1r,4r)-4-(4-(trifluoromethyl)pyridin-3-yl)cyclohexyl)-1,8-naphthyridin-2(1H)-one (247)

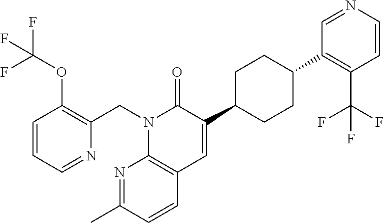 [SMILES: Cc1ccc2cc([C@H]3CC[C@H](c4cnccc4C(F)(F)F)CC3)c(=O)n(Cc3ncccc3OC(F)(F)F)c2n1]

Step 1: 7-methyl-1-((3-(trifluoromethoxy)pyridin-2-yl)methyl)-3-((1r,4r)-4-(4-(trifluoromethyl)pyridin-3-yl)cyclohexyl)-1,8-naphthyridin-2(1H)-one: Followed the General Procedure AP using 7-methyl-3-((1r,4r)-4-(4-(trifluoromethyl)pyridin-3-yl)cyclohexyl)-1,8-naphthyridin-2(1H)-one (50 mg, 0.129 mmol, 1 equiv.) and 2-(chloromethyl)-3-(trifluoromethoxy)pyridine (32.7 mg, 0.155 mmol, 1.2 equiv.) as the starting materials to give 7-methyl-1-((3-(trifluoromethoxy)pyridin-2-yl)methyl)-3-((1r,4r)-4-(4-(trifluoromethyl)pyridin-3-yl)cyclohexyl)-1,8-naphthyridin-2(1H)-one (54 mg, 73.78%) as a white solid. ¹H NMR (400 MHz, Chloroform-d) δ 8.82 (s, 1H), 8.62 (d, J=5.2 Hz, 1H), 8.29 (dd, J=4.7, 1.4 Hz, 1H), 7.74 (d, J=7.8 Hz, 1H), 7.62-7.51 (m, 3H), 7.17 (dd, J=8.3, 4.7 Hz, 1H), 7.00 (d, J=7.8 Hz, 1H), 6.03 (s, 2H), 3.14 (tt, J=11.9, 3.1 Hz, 1H), 3.04 (t, J=12.2 Hz, 1H), 2.49 (s, 3H), 2.23-2.15 (m, 2H), 2.06-2.01 (m, 2H), 1.89-1.75 (m, 2H), 1.58 (qd, J=12.6, 3.3 Hz, 2H). MS m/z: 563.10 [M+H]⁺.

### 1-((3-Cyclopropylpyridin-2-yl)methyl)-7-methyl-3-((1r,4r)-4-(4-(trifluoromethyl)pyridin-3-yl)cyclohexyl)-1,8-naphthyridin-2(1H)-one (248)

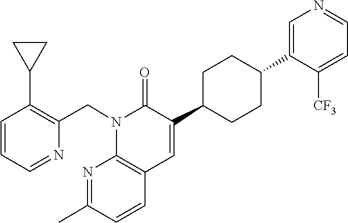 [SMILES: Cc1ccc2cc([C@H]3CC[C@H](c4cnccc4C(F)(F)F)CC3)c(=O)n(Cc3ncccc3C3CC3)c2n1]

Step 1: 1-((3-cyclopropylpyridin-2-yl)methyl)-7-methyl-3-((1r,4r)-4-(4-(trifluoromethyl)pyridin-3-yl)cyclohexyl)-1,8-naphthyridin-2(1H)-one: Followed the General Procedure AP using 7-methyl-3-((1r,4r)-4-(4-(trifluoromethyl)pyridin-3-yl)cyclohexyl)-1,8-naphthyridin-2(1H)-one (50 mg, 0.129 mmol, 1 equiv) and 2-(chloromethyl)-3-cyclopropylpyridine (32.4 mg, 0.194 mmol, 1.5 equiv.) as the starting materials to give 1-((3-cyclopropylpyridin-2-yl)methyl)-7-methyl-3-((1r,4r)-4-(4-(trifluoromethyl)pyridin-3-yl)cyclohexyl)-1,8-naphthyridin-2(1H)-one (30.1 mg, 44.2%) as a white solid. ¹H NMR (400 MHz, Chloroform-d) δ 8.81 (s, 1H), 8.59 (d, J=5.1 Hz, 1H), 8.40-8.15 (m, 1H), 7.75 (d, J=7.8 Hz, 1H), 7.58-7.44 (m, 3H), 7.25-7.5 (m, 1H), 7.00 (d, J=7.8 Hz, 1H), 6.19 (s, 2H), 3.15 (t, J=12.1 Hz, 1H), 3.02 (t, J=12.3 Hz, 1H), 2.50 (s, 3H), 2.25-2.15 (m, 3H), 2.05-1.95 (s, 2H), 1.87-1.79 (m, 2H), 1.60-1.56 (m, 2H), 1.11-0.98 (m, 2H), 0.85-0.76 (m, 2H). MS m/z: 519.15 [M+H]⁺.

### 3-Methyl-7-((1r,4r)-4-(5-methylisothiazol-4-yl)cyclohexyl)-5-((3-(trifluoromethyl)pyridin-2-yl)methyl)pyrido[2,3-b]pyrazin-6(5H)-one (249)

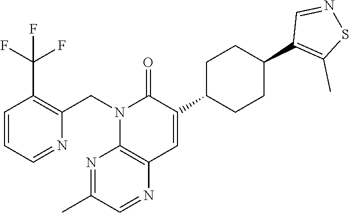 [SMILES: Cc1cnc2cc([C@H]3CC[C@H](c4cnsc4C)CC3)c(=O)n(Cc3ncccc3C(F)(F)F)c2n1]

Step 1: 3-methyl-7-((1r,4r)-4-(5-methylisothiazol-4-yl)cyclohexyl)-5-((3-(trifluoromethyl)pyridin-2-yl)methyl)pyrido[2,3-b]pyrazin-6(5H)-one: Followed the General Procedure AP using 3-methyl-7-((1r,4r)-4-(5-methylisothiazol-4-yl)cyclohexyl)pyrido[2,3-b]pyrazin-6(5H)-one (50 mg, 0.147 mmol, 1 equiv.) and 2-(chloromethyl)-3-(trifluoromethyl)pyridine hydrochloride (50.9 mg, 0.22 mmol, 1.5 equiv.) as the starting materials to give 3-methyl-7-((1 r,4r)-4-(5-methylisothiazol-4-yl)cyclohexyl)-5-((3-(trifluoromethyl)pyridin-2-yl)methyl)pyrido[2,3-b]pyrazin-6(5H)-one (20.4 mg, 27.5%) as a white solid. ¹H NMR (400 MHz, Chloroform-d) δ 8.46-8.38 (m, 1H), 8.31 (d, J=9.7 Hz, 2H), 8.01-7.91 (m, 1H), 7.81 (s, 1H), 7.25-7.21 (m, 1H), 6.03 (s, 2H), 3.10 (tt, J=12.0, 3.6 Hz, 11H), 2.70 (tt, J=12.2, 3.6 Hz, I H), 2.52 (s, 3H), 2.48 (s, 3H), 2.21-2.13 (m, 2H), 2.04-1.95 (m, 2H), 1.73-1.66 (m, 2H), 1.65-1.51 (m, 2H). MS m/z: 500.15 [M+H]⁺.

### 5-((3-(1,1-Difluoroethyl)pyridin-2-yl)methyl)-7-((1r,4r)-4-(2,6-difluorophenyl)cyclohexyl)-3-methylpyrido[2,3-b]pyrazin-6(5H)-one (250)

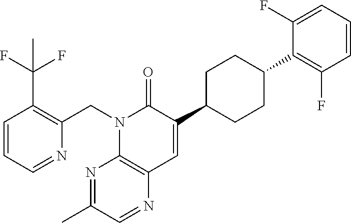 [SMILES: Cc1cnc2cc([C@H]3CC[C@H](c4c(F)cccc4F)CC3)c(=O)n(Cc3ncccc3C(C)(F)F)c2n1]

Step 1: 7-((1r,4r)-4-(2,6-difluorophenyl)cyclohexyl)-3-methylpyrido[2,3-b]pyrazin-6(5H)-one: Followed the General Procedure D using 2-((1r,4r)-4-(2,6-difluorophenyl)cyclohexyl)acetate (100 mg, 0.354 mmol, 1 equiv.) and 3-amino-5-methylpyrazine-2-carbaldehyde (48.5 mg, 0.354 mmol, 1 equiv.) as the starting materials to give 7-((1r,4r)-4-(2,6-difluorophenyl)cyclohexyl)-3-methylpyrido[2,3-b]pyrazin-6(5H)-one (50 mg, 39%) as a white solid. MS m/z: 356 [M+H]⁺.

Step 2: 5-((3-(1,1-difluoroethyl)pyridin-2-yl)methyl)-7-((1r,4r)-4-(2,6-difluorophenyl)cyclohexyl)-3-methylpyrido[2,3-b]pyrazin-6(5H)-one: Followed the General Procedure AP using 7-((1r,4r)-4-(2,6-difluorophenyl)cyclohexyl)-3-methylpyrido[2,3-b]pyrazin-6(5H)-one (50 mg, 0.141 mmol, 1 equiv.) and 2-(chloromethyl)-3-(1,1-difluoroethyl)pyridine (30 mg, 0.141 mmol, 1 equiv.) as the starting materials to give 5-((3-(1,1-difluoroethyl)pyridin-2-yl)methyl)-7-((1r,4r)-4-(2,6-difluorophenyl)cyclohexyl)-3-methylpyrido[2,3-b]pyrazin-6(5H)-one (7.6 mg, 10.6%) as a white solid. ¹H NMR (400 MHz, Chloroform-d) δ 8.33 (s, 2H), 7.86-7.78 (m, 2H), 7.22-7.12 (m, 1H), 7.12-7.05 (m, 1H), 6.82 (t, J=8.6 Hz, 2H), 5.96 (d, J=2.4 Hz, 2H), 3.18-3.05 (m, 2H), 2.53 (s, 3H), 2.25 (t, J=18.3 Hz, 3H), 2.17-2.05 (m, 4H), 1.93-1.85 (m, 2H), 1.56-1.48 (m, 2H). MS m/z: 511.2 [M+H]⁺.

### 7-Methyl-1-((3-(trifluoromethyl)pyridin-2-yl)methyl)-3-((1r,4r)-4-(4-(trifluoromethyl)pyridin-3-yl)cyclohexyl)-1,8-naphthyridin-2(1H)-one (251)

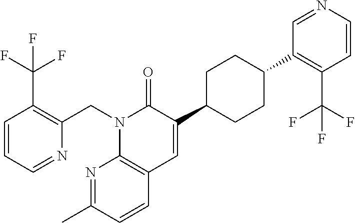 [SMILES: Cc1ccc2cc([C@H]3CC[C@H](c4cnccc4C(F)(F)F)CC3)c(=O)n(Cc3ncccc3C(F)(F)F)c2n1]

Step 1: 7-methyl-1-((3-(trifluoromethyl)pyridin-2-yl)methyl)-3-((1r,4r)-4-(4-(trifluoromethyl)pyridin-3-yl)cyclohexyl)-1,8-naphthyridin-2(1H)-one: Followed the General Procedure AP using 7-methyl-3-((1r,4r)-4-(4-(trifluoromethyl)pyridin-3-yl)cyclohexyl)-1,8-naphthyridin-2(1H)-one (50 mg, 0.129 mmol, 1 equiv) and 2-(chloromethyl)-3-(trifluoromethyl)pyridine hydrochloride (44.9 mg, 0.194 mmol, 1.5 equiv.) as the starting materials to give 7-methyl-1-((3-(trifluoromethyl)pyridin-2-yl)methyl)-3-((1r,4r)-4-(4-(trifluoromethyl)pyridin-3-yl)cyclohexyl)-1,8-naphthyridin-2(1H)-one (17.7 mg, 24.0%) as a white solid. ¹H NMR (400 MHz, Chloroform-d) δ 8.83 (s, 1H), 8.62 (d, J=5.2 Hz, 1H), 8.48-8.42 (m, 1H), 8.00-7.93 (m, 1H), 7.75 (d, J=7.8 Hz, 1H), 7.60-7.53 (m, 2H), 7.21 (dd, J=7.9, 4.9 Hz, 1H), 6.99 (d, J=7.8 Hz, 1H), 6.11 (s, 2H), 3.15 (t, J=12.1, 1H), 3.05 (t, J=12.2 Hz, 1H), 2.47 (s, 3H), 2.20 (d, J=12.8 Hz, 2H), 2.07-1.99 (m, 2H), 1.89-1.75 (m, 2H), 1.60 (qd, J=12.6, 3.3 Hz, 2H). MS m/z: 547.15 [M+H]⁺.

### 1-((3-Cyclopropylpyridin-2-yl)methyl)-3-((1r,4r)-4-(2-methoxy-4-methylpyridin-3-yl)cyclohexyl)-7-methyl-1,8-naphthyridin-2(1H)-one (252)

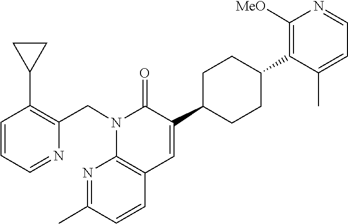 [SMILES: COc1nccc(C)c1[C@H]1CC[C@H](c2cc3ccc(C)nc3n(Cc3ncccc3C3CC3)c2=O)CC1]

Step 1: 1-((3-cyclopropylpyridin-2-yl)methyl)-3-((1r,4r)-4-(2-methoxy-4-methylpyridin-3-yl)cyclohexyl)-7-methyl-1,8-naphthyridin-2(1H)-one: Followed the General Procedure AP using 3-((1r,4r)-4-(2-methoxy-4-methylpyridin-3-yl)cyclohexyl)-7-methyl-1,8-naphthyridin-2(1H)-one (50 mg, 0.108 mmol, 1 equiv.) and 2-(chloromethyl)-3-cyclopropylpyridine (30.65 mg, 0.162 mmol, 1.5 equiv) as the starting materials to give 1-((3-cyclopropylpyridin-2-yl)methyl)-3-((1r,4r)-4-(2-methoxy-4-methylpyridin-3-yl)cyclohexyl)-7-methyl-1,8-naphthyridin-2(1H)-one (18.8 mg, 27.0%) as a white solid. ¹H NMR (400 MHz, Chloroform-d) δ 8.28 (s, 1H), 7.86 (d, J=5.1 Hz, 1H), 7.74 (d, J=7.8 Hz, 1H), 7.54 (s, 2H), 7.14 (s, 1H), 6.99 (d, J=7.9 Hz, 1H), 6.66 (d, J=5.1 Hz, 1H), 6.20 (s, 2H), 3.92 (s, 3H), 3.16-3.08 (m, 1H), 3.00-2.85 (m, 1H), 2.50 (s, 3H), 2.33 (s, 5H), 2.23-2.10 (m, 3H), 1.66 (d, J=12.7 Hz, 2H), 1.52-1.47 (m, 2H), 1.08-1.03 (m, 2H), 0.80 (d, J=5.5 Hz, 2H). MS m/z: 495.2 [M+H]⁺.

The following compounds were prepared in an analogous manner to those prepared above.

### 3-Methyl-7-[(1r,4r)-4-(5-methyl-4-isothiazolyl)cyclohexyl]-5-[(3-trifluoromethoxy-2-pyridyl)methyl]-1,4,5-triaza-6(5H)-naphthalenone (253)

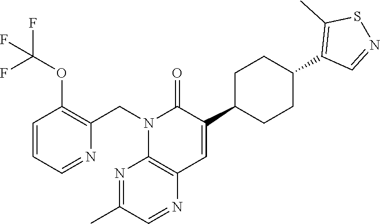 [SMILES: Cc1cnc2cc([C@H]3CC[C@H](c4cnsc4C)CC3)c(=O)n(Cc3ncccc3OC(F)(F)F)c2n1]

3-methyl-7-((1r,4r)-4-(5-methylisothiazol-4-yl)cyclohexyl)-5-((3-(trifluoromethoxy)pyridin-2-yl)methyl)pyrido[2,3-b]pyrazin-6(5H)-one: Followed the general procedure AP using 3-methyl-7-((1r,4r)-4-(5-methylisothiazol-4-yl)cyclohexyl)pyrido[2,3-b]pyrazin-6(5H)-one (35 mg, 0.103 mmol, 1 equiv.) and 2-(chloromethyl)-3-(trifluoromethoxy)pyridine hydrochloride (127 mg, 0.515 mmol, 5 equiv.) as the starting materials to give 3-methyl-7-((1r,4r)-4-(5-methylisothiazol-4-yl)cyclohexyl)-5-((3-(trifluoromethoxy)pyridin-2-yl)methyl)pyrido[2,3-b]pyrazin-6(5H)-one (29.3 mg, 54.8%) as a light yellow solid. ^{l}H NMR (400 MHz, Chloroform-d) δ 8.31 (d, J=10.8 Hz, 2H), 8.27-8.26 (m, 1H), 7.79 (s, 1H), 7.61-7.58 (m, 1H), 7.22-7.19 (m, 1H), 5.95 (s, 2H), 3.13-3.06 (m, 1H), 2.74-2.66 (m, 1H), 2.53 (s, 3H), 2.48 (s, 3H), 2.18-2.14 (m, 2H), 2.02-1.97 m, 2H), 1.77-1.67 (m, 2H), 1.62-1.51 (m, 2H). MS m/z: 516.15 [M+H]⁺.

### 1-{[3-(1,1-Difluoroethyl)-2-pyridyl]methyl}-7-methyl-3-{(1r,4r)-4-[4-(trifluoromethyl)-3-pyridyl]cyclohexyl}-1,8-diaza-2(1H)-naphthalenone (254)

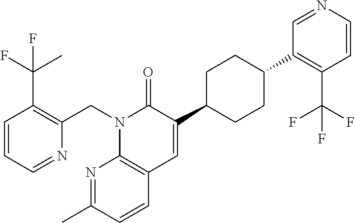 [SMILES: Cc1ccc2cc([C@H]3CC[C@H](c4cnccc4C(F)(F)F)CC3)c(=O)n(Cc3ncccc3C(C)(F)F)c2n1]

Step 1: 7-methyl-3-((1r,4r)-4-(4-(trifluoromethyl)pyridin-3-yl)cyclohexyl)-1,8-naphthyridin-2(1H)-one: Followed the general procedure D using ethyl 2-(4-(4-(trifluoromethyl)pyridin-3-yl)cyclohexyl)acetate (280 mg, 1.04 mmol, 1 equiv.) and 2-amino-6-methylnicotinaldehyde (215 mg, 1.57 mmol, 1.5 equiv.) as the starting materials to give 7-methyl-3-((1r,4r)-4-(4-(trifluoromethyl)pyridin-3-yl)cyclohexyl)-1,8-naphthyridin-2(1H)-one (100 mg, 28%) as an off-white solid. MS m/z: 388 [M+H]⁺.

Step 2: 1-((3-(1,1-difluoroethyl)pyridin-2-yl)methyl)-7-methyl-3-((1r,4r)-4-(4-(trifluoromethyl)pyridin-3-yl)cyclohexyl)-1,8-naphthyridin-2(1H)-one: Followed the general procedure AP using 7-methyl-3-((1r,4r)-4-(4-(trifluoromethyl)pyridin-3-yl)cyclohexyl)-1,8-naphthyridin-2(1H)-one (100 mg, 0.257 mmol, 1.0 equiv.) and 2-(chloromethyl)-3-(1,1-difluoroethyl)pyridine (75.4 mg, 0.386 mmol, 1.5 equiv.) as the starting materials to give 1-((3-(1,1-difluoroethyl)pyridin-2-yl)methyl)-7-methyl-3-((1r,4r)-4-(4-(trifluoromethyl)pyridin-3-yl)cyclohexyl)-1,8-naphthyridin-2(1H)-one (20.1 mg, 28.7%) as a pink solid. ¹H NMR (400 MHz, Chloroform-d) δ 8.81 (s, 1H), 8.61 (d, J=5.2 Hz, 1H), 8.41-8.35 (m, 1H), 7.85 (d, J=7.8 Hz, 1H), 7.76 (d, J=7.8 Hz, 1H), 7.58-7.51 (m, 2H), 7.20 (dd, J=7.8, 4.9 Hz, 1H), 7.00 (d, J=7.8 Hz, 1H), 6.04 (s, 2H), 3.14 (t, J=12.1 Hz, 1H), 3.04 (t, J=12.1 Hz, 1H), 2.48 (s, 3H), 2.30 (d, J=18.3 Hz, 3H), 2.20 (d, J=12.1 Hz, 2H), 2.02 (d, J=12.9 Hz, 2H), 1.85-1.80 (m, 2H), 1.62 (dd, J=12.5, 3.3 Hz, 2H). MS m/z: 543.20 [M+H]⁺.

### 7-Methyl-3-[(1r,4r)-4-(2-fluoro-4-methyl-3-pyridyl)cyclohexyl]-1-[(3-trifluoromethoxy-2-pyridyl)methyl]-1,8-diaza-2(1H)-naphthalenone (255)

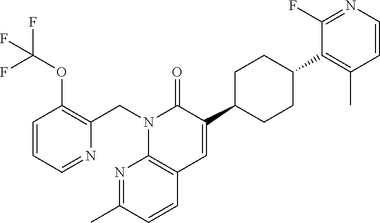 [SMILES: Cc1ccc2cc([C@H]3CC[C@H](c4c(C)ccnc4F)CC3)c(=O)n(Cc3ncccc3OC(F)(F)F)c2n1]

3-((1r,4r)-4-(2-fluoro-4-methylpyridin-3-yl)cyclohexyl)-7-methyl-1-((3-(trifluoromethoxy)pyridin-2-yl)methyl)-1,8-naphthyridin-2(1H)-one: Followed the general Procedure AP using 3-((1r,4r)-4-(2-fluoro-4-methylpyridin-3-yl)cyclohexyl)-7-methyl-1,8-naphthyridin-2(1H)-one (30 mg, 0.09 mmol, 1 equiv.) and 2-(chloromethyl)-3-(trifluoromethoxy)pyridine (20 mg, 0.10 mmol, 1.2 equiv.) as the starting materials to give 3-((1r,4r)-4-(2-fluoro-4-methylpyridin-3-yl)cyclohexyl)-7-methyl-1-((3-(trifluoromethoxy)pyridin-2-yl)methyl)-1,8-naphthyridin-2(1H)-one (21.2 mg, 46.6%) as a white solid. ¹H NMR (400 MHz, Chloroform-d) δ 8.32 (dd, J=4.8, 1.4 Hz, 1H), 7.89 (d, J=5.0 Hz, 1H), 7.74 (d, J=7.8 Hz, 1H), 7.66-7.58 (m, 1H), 7.52 (s, 1H), 7.21 (dd, J=8.3, 4.8 Hz, 1H), 6.99 (d, J=7.8 Hz, 1H), 6.94 (d, J=4.3 Hz, 1H), 6.04 (s, 2H), 3.10 (t, J=12.1 Hz, 1H), 2.89 (t, J=12.1 Hz, 1H), 2.49 (s, 3H), 2.40 (s, 3H), 2.22-2.07 (m, 4H), 1.81-1.76 (m, 2H), 1.63-1.48 (m, 2H). MS m/z: 527.15 [M+H]⁺.

### 1-{[3-(1,1-Difluoroethyl)-2-pyridyl]methyl}-7-methyl-3-[(1r,4r)-4-(2-methoxy-4-methyl-3-pyridyl)cyclohexyl]-1,8-diaza-2(1H)-naphthalenone (256)

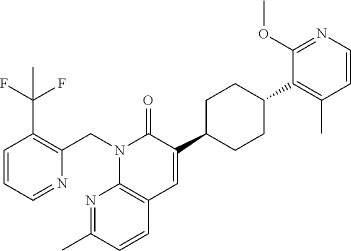 [SMILES: COc1nccc(C)c1[C@H]1CC[C@H](c2cc3ccc(C)nc3n(Cc3ncccc3C(C)(F)F)c2=O)CC1]

1-((3-(1,1-difluoroethyl)pyridin-2-yl)methyl)-3-((1r,4r)-4-(2-methoxy-4-methylpyridin-3-yl)cyclohexyl)-7-methyl-1,8-naphthyridin-2(1H)-one: Followed the general procedure AP using 3-((1r,4r)-4-(2-methoxy-4-methylpyridin-3-yl)cyclohexyl)-7-methyl-1,8-naphthyridin-2(1H)-one (50 mg, 0.138 mmol, 1 equiv.) and 2-(chloromethyl)-3-(1,1-difluoroethyl)pyridine (39.5 mg, 0.207 mmol, 1.5 equiv.) as the starting materials to give 1-((3-(1,1-difluoroethyl)pyridin-2-yl)methyl)-3-((1r,4r)-4-(2-methoxy-4-methylpyridin-3-yl)cyclohexyl)-7-methyl-1,8-naphthyridin-2(1H)-one (19.3 mg, 25.9%) as a white solid. ¹H NMR (400 MHz, Chloroform-d) δ 8.34 (d, J=4.7 Hz, 1H), 7.92 (d, J=5.1 Hz, 1H), 7.80 (dd, J=8.0, 1.7 Hz, 1H), 7.74 (d, J=7.8 Hz, 1H), 7.53 (s, 1H), 7.17-7.13 (m, 1H), 6.99 (d, J=7.9 Hz, 1H), 6.75 (s, 1H), 6.03 (s, 2H), 4.03 (s, 3H), 3.14 (t, J=11.9 Hz, 1H), 2.95 (s, 1H), 2.48 (s, 3H), 2.38 (s, 3H), 2.27 (t, J=18.3 Hz, 5H), 2.12 (d, J=12.7 Hz, 2H), 1.67 (d, J=13.0 Hz, 2H), 1.46 (d, J=9.9 Hz, 2H). MS m/z: 519.25[M+H]⁺.

### 1-[(3-Cyclopropyl-2-pyrazinyl)methyl]-7-methyl-3-[(1r,4r)-4-(2-fluoro-6-tolyl)cyclohexyl]-1,8-diaza-2(1H)-naphthalenone (257)

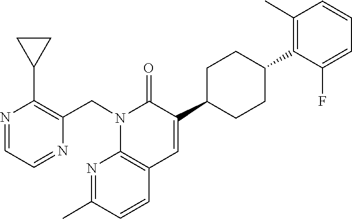 [SMILES: Cc1ccc2cc([C@H]3CC[C@H](c4c(C)cccc4F)CC3)c(=O)n(Cc3nccnc3C3CC3)c2n1]

1-((3-cyclopropylpyrazin-2-yl)methyl)-3-((1r,4r)-4-(2-fluoro-6-methylphenyl)cyclohexyl)-7-methyl-1,8-naphthyridin-2(1H)-one: Followed the general Procedure AP using 3-((1r,4r)-4-(2-fluoro-6-methylphenyl)cyclohexyl)-7-methyl-1,8-naphthyridin-2(1H)-one (30 mg, 0.086 mmol, 1 equiv.) and 2-(chloromethyl)-3-cyclopropylpyrazine (21.6 mg, 0.129 mmol, 1.5 equiv.) as the starting materials to give 1-((3-cyclopropylpyrazin-2-yl)methyl)-3-((1r,4r)-4-(2-fluoro-6-methylphenyl)cyclohexyl)-7-methyl-1,8-naphthyridin-2(1H)-one (5.6 mg, 13.5%) as a white solid. ¹H NMR (400 MHz, Chloroform-d) δ 8.22 (d, J=2.6 Hz, 1H), 8.09 (d, J=2.6 Hz, 1H), 7.75 (d, J=7.8 Hz, 1H), 7.54 (s, 1H), 7.06-6.98 (m, 2H), 6.94-6.82 (m, 2H), 6.14 (s, 2H), 3.13 (t, J=12.0 Hz, 1H), 2.90 (t, J=12.4 Hz, 1H), 2.50 (s, 3H), 2.37 (s, 4H), 2.16 (t, J=12.9 Hz, 4H), 1.86 (s, 2H), 1.53 (d, J=12.4 Hz, 2H), 1.26-1.24 (m, 2H), 1.17-1.14 (m, 2H). MS m/z: 483.20 [M+H]⁺.

### 1-{[3-(1,1-Difluoroethyl)-2-pyridyl]methyl}-7-methyl-3-[(1r,4r)-4-(2-fluoro-4-methyl-3-pyridyl)cyclohexyl]-1,8-diaza-2(1H)-naphthalenone (258)

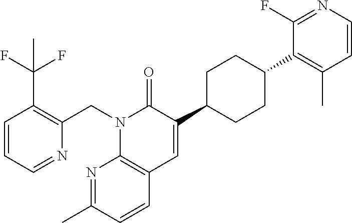 [SMILES: Cc1ccc2cc([C@H]3CC[C@H](c4c(C)ccnc4F)CC3)c(=O)n(Cc3ncccc3C(C)(F)F)c2n1]

Step 1: ethyl 2-(4-(2-fluoro-4-methylpyridin-3-yl)cyclohex-3-en-1-yl)acetate: Followed the general Procedure AL using ethyl 2-(4-(4,4,5,5-tetramethyl-1,3,2-dioxaborolan-2-yl)cyclohex-3-en-1-yl)acetate (1 g, 3.40 mmol, 1 equiv.) and 3-bromo-2-fluoro-4-methylpyridine (707 mg, 3.74 mmol, 1.1 equiv.) as the starting materials to give ethyl 2-(4-(2-fluoro-4-methylpyridin-3-yl)cyclohex-3-en-1-yl)acetate (800 mg, 85%) as a colorless oil. MS m/z: 278 [M+H]⁺.

Step 2: ethyl 2-(4-(2-fluoro-4-methylpyridin-3-yl)cyclohexyl)acetate: Followed the general Procedure AR using ethyl 2-(4-(2-fluoro-4-methylpyridin-3-yl)cyclohex-3-en-1-yl)acetate (800 mg, 2.89 mmol, 1 equiv.) as the starting material to give the crude product ethyl 2-(4-(2-fluoro-4-methylpyridin-3-yl)cyclohexyl)acetate (780 mg, crude) as a colorless oil. MS m/z: 280 [M+H]⁺.

Step 3: 3-((1r,4r)-4-(2-fluoro-4-methylpyridin-3-yl)cyclohexyl)-7-methyl-1,8-naphthyridin-2(1H)-one: Followed the general Procedure D using ethyl 2-(4-(2-fluoro-4-methylpyridin-3-yl)cyclohexyl)acetate (300 mg, 1.08 mmol, 1 equiv.) and 2-amino-6-methylnicotinaldehyde (176 mg, 1.30 mmol, 1.2 equiv.) as the starting materials to give 3-((1r,4r)-4-(2-fluoro-4-methylpyridin-3-yl)cyclohexyl)-7-methyl-1,8-naphthyridin-2(1H)-one (120 mg, 31%) as a colorless oil. MS m/z: 352 [M+H]⁺.

Step 4: 1-((3-(1,1-difluoroethyl)pyridin-2-yl)methyl)-3-((1r,4r)-4-(2-fluoro-4-methylpyridin-3-yl)cyclohexyl)-7-methyl-1,8-naphthyridin-2(1H)-one: Followed the general Procedure AP using 2-(chloromethyl)-3-(1,1-difluoroethyl)pyridine (29 mg, 0.13 mmol, 1 equiv.) and 3-bromo-2-fluoro-4-methylpyridine (56 mg, 0.16 mmol, 1.2 equiv.) as the starting materials to give 1-((3-(1,1-difluoroethyl)pyridin-2-yl)methyl)-3-((1r,4r)-4-(2-fluoro-4-methylpyridin-3-yl)cyclohexyl)-7-methyl-1,8-naphthyridin-2(1H)-one (8.1 mg, 12.3%) as a white solid. ¹H NMR (400 MHz, Chloroform-d) δ 8.49 (s, 1H), 7.98 (s, 1H), 7.89 (d, J=4.9 Hz, 1H), 7.77 (dd, J=7.8, 4.0 Hz, 1H), 7.55 (d, J=4.0 Hz, 1H), 7.31 (s, 1H), 7.12-6.86 (m, 2H), 6.07 (s, 2H), 3.10 (t, J=11.6 Hz, 1H), 2.99-2.84 (m, 1H), 2.49 (s, 3H), 2.40 (s, 3H), 2.36-2.22 (m, 3H), 2.22-2.06 (m, 4H), 1.84-1.78 (m, 2H), 1.59-1.53 (m, 2H). MS m/z: 507.20 [M+H]⁺.

### 7-Methyl-3-[(1r,4r)-4-(2-fluoro-4-methyl-3-pyridyl)cyclohexyl]-1-{[3-(2,2,2-trifluoroethyl)-2-pyridyl]methyl}-1,8-diaza-2(1H)-naphthalenone (259)

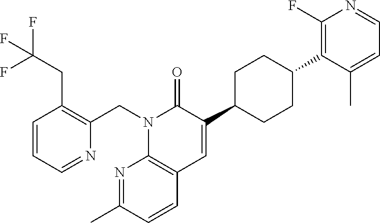 [SMILES: Cc1ccc2cc([C@H]3CC[C@H](c4c(C)ccnc4F)CC3)c(=O)n(Cc3ncccc3CC(F)(F)F)c2n1]

3-((1r,4r)-4-(2-fluoro-4-methylpyridin-3-yl)cyclohexyl)-7-methyl-1-((3-(2,2,2-trifluoroethyl)pyridin-2-yl)methyl)-1,8-naphthyridin-2(1H)-one: Followed the general Procedure AP using 3-((1r,4r)-4-(2-fluoro-4-methylpyridin-3-yl)cyclohexyl)-7-methyl-1,8-naphthyridin-2(1H)-one (30 mg, 0.09 mmol, 1 equiv.) and 2-(chloromethyl)-3-(2,2,2-trifluoroethyl)pyridine (20 mg, 0.10 mmol, 1.2 equiv.) as the starting materials to give 3-((1r,4r)-4-(2-fluoro-4-methylpyridin-3-yl)cyclohexyl)-7-methyl-1-((3-(2,2,2-trifluoroethyl)pyridin-2-yl)methyl)-1,8-naphthyridin-2(1H)-one (16.8 mg, 35.4%) as a white solid. ¹H NMR (400 MHz, Chloroform-d) δ 8.39 (d, J=3.8 Hz, 1H), 7.89 (d, J=5.0 Hz, 1H), 7.73 (d, J=7.8 Hz, 1H), 7.62 (d, J=7.7 Hz, 1H), 7.50 (s, 1H), 7.20-7.09 (m, 1H), 7.00 (d, J=7.8 Hz, 1H), 6.94 (d, J=4.9 Hz, 1H), 5.94 (s, 2H), 3.83 (q, J=10.6 Hz, 2H), 3.10 (t, J=12.2 Hz, 1H), 2.90 (t, J=12.2 Hz, 1H), 2.52 (s, 3H), 2.40 (s, 3H), 2.23-2.06 (m, 4H), 1.85-1.76 (m, 2H), 1.57-1.47 (m, 2H). MS m/z: 525.20 [M+H]⁺.

### 7-Methyl-3-[(1r,4r)-4-(2-methoxy-4-methyl-3-pyridyl)cyclohexyl]-1-{[3-(2,2,2-trifluoroethyl)-2-pyridyl]methyl}-1,8-diaza-2(1H)-naphthalenone (260)

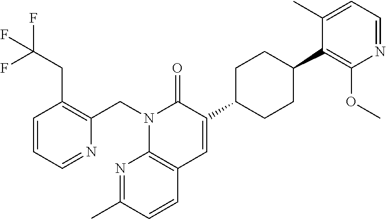 [SMILES: COc1nccc(C)c1[C@H]1CC[C@H](c2cc3ccc(C)nc3n(Cc3ncccc3CC(F)(F)F)c2=O)CC1]

3-((1r,4r)-4-(2-methoxy-4-methylpyridin-3-yl)cyclohexyl)-7-methyl-1-((3-(2,2,2-trifluoroethyl)pyridin-2-yl)methyl)-1,8-naphthyridin-2(1H)-one: Followed the general procedure AP using 3-((1r,4r)-4-(2-methoxy-4-methylpyridin-3-yl)cyclohexyl)-7-methyl-1,8-naphthyridin-2(1H)-one (50 mg, 0.138 mmol, 1 equiv.) and 2-(chloromethyl)-3-(2,2,2-trifluoroethyl)pyridine hydrochloride (57.6 mg, 0.276 mmol, 2 equiv.) as the starting materials to give 3-((1r,4r)-4-(2-methoxy-4-methylpyridin-3-yl)cyclohexyl)-7-methyl-1-((3-(2,2,2-trifluoroethyl)pyridin-2-yl)methyl)-1,8-naphthyridin-2(1H)-one (29.8 mg, 38.8%) as a white solid. ¹H NMR (400 MHz, Chloroform-d) δ 8.45 (s, 1H), 7.96 (s, 1H), 7.74 (d, J=7.8 Hz, 2H), 7.51 (s, 1H), 7.22 (d, J=7.91 Hz, 1H), 7.01 (d, J=8.0 Hz, 1H), 6.81 (s, 1H), 5.98 (s, 2H), 4.12 (s, 3H), 3.89-3.80 (m, 2H), 3.09 (t, J=12.1 Hz, 1H), 2.96 (t, J=12.1 Hz, 1H), 2.53 (s, 3H), 2.40 (s, 3H), 2.30 (d, J=13.1 Hz, 2H), 2.11 (d, J=12.5 Hz, 2H), 1.71-1.68 (s, 2H), 1.50-1.48 (s, 2H). MS m/z: 537.25 [M+H]⁺.

### 7-Methyl-3-[(1r,4r)-4-(2-methoxy-4-methyl-3-pyridyl)cyclohexyl]-1-[(3-trifluoromethoxy-2-pyridyl)methyl]-1,8-diaza-2(1H)-naphthalenone (261)

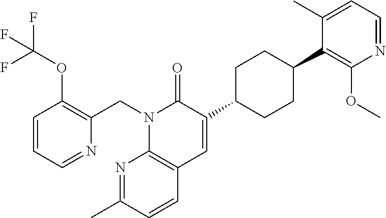 [SMILES: COc1nccc(C)c1[C@H]1CC[C@H](c2cc3ccc(C)nc3n(Cc3ncccc3OC(F)(F)F)c2=O)CC1]

1-3-((1r,4r)-4-(2-methoxy-4-methylpyridin-3-yl)cyclohexyl)-7-methyl-1-((3-(trifluoromethoxy) pyridin-2-yl)methyl)-1,8-naphthyridin-2(1H)-one: Followed the general procedure AP using 3-((1r,4r)-4-(2-methoxy-4-methylpyridin-3-yl)cyclohexyl)-7-methyl-1,8-naphthyridin-2(1H)-one (50 mg, 0.138 mmol, 1 equiv.) and 2-(chloromethyl)-3-(trifluoromethoxy)pyridine (43.6 mg, 0.207 mmol, 1.5 equiv.) as the starting materials to give 3-((1r,4r)-4-(2-methoxy-4-methylpyridin-3-yl)cyclohexyl)-7-methyl-1-((3-(trifluoromethoxy)pyridin-2-yl)methyl)-1,8-naphthyridin-2(1H)-one (34.8 mg, 44.8%) as a white solid. ¹H NMR (400 MHz, Chloroform-d) δ 8.31-8.27 (m, 1H), 7.92 (d, J=5.2 Hz, 1H), 7.74 (d, J=7.8 Hz, 1H), 7.59-7.49 (m, 2H), 7.19-7.15 (m, 1H), 6.99 (d, J=7.8 Hz, 1H), 6.75 (s, 1H), 6.03 (s, 2H), 4.03 (s, 3H), 3.11 (t, J=12.0 Hz, 1H), 2.95 (s, 1H), 2.49 (s, 3H), 2.35 (d, J=16.1 Hz, 5H), 2.12 (d, J=12.5 Hz, 2H), 1.67 (d, J=12.7 Hz, 2H), 1.49-1.44 (m, 2H). MS m/z: 539.20 [M+H]⁺.

### 7-Methyl-3-[(1r,4r)-4-(2-fluoro-4-methyl-3-pyridyl)cyclohexyl]-1-{[3-(trifluoromethyl)-2-pyridyl]methyl}-1,8-diaza-2(1H)-naphthalenone (262)

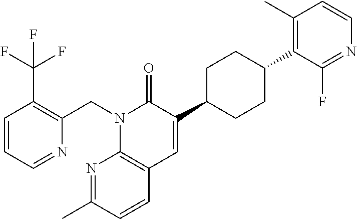 [SMILES: Cc1ccc2cc([C@H]3CC[C@H](c4c(C)ccnc4F)CC3)c(=O)n(Cc3ncccc3C(F)(F)F)c2n1]

3-((1r,4r)-4-(2-fluoro-4-methylpyridin-3-yl)cyclohexyl)-7-methyl-1-((3-(trifluoromethyl)pyridin-2-yl)methyl)-1,8-naphthyridin-2(1H)-one: Followed the general Procedure AP using 3-((1r,4r)-4-(2-fluoro-4-methylpyridin-3-yl)cyclohexyl)-7-methyl-1,8-naphthyridin-2(1H)-one (40 mg, 0.120 mmol, 1 equiv.) and 2-(chloromethyl)-3-(trifluoromethoxy)pyridine (25 mg, 0.132 mmol, 1.1 equiv.) as the starting materials to give 3-((1r,4r)-4-(2-fluoro-4-methylpyridin-3-yl)cyclohexyl)-7-methyl-1-((3-(trifluoromethyl)pyridin-2-yl)methyl)-1,8-naphthyridin-2(1H)-one (31.8 mg, 53.3%) as a white solid. ¹H NMR (400 MHz, DMSO-d₆) δ 8.51 (d, J=4.4 Hz, 1H), 8.21 (d, J=7.7 Hz, 1H), 8.06 (d, J=7.8 Hz, 1H), 7.92 (d, J=4.8 Hz, 1H), 7.84 (s, 1H), 7.45 (dd, J=7.7, 5.0 Hz, 1H), 7.22-7.10 (m, 2H), 5.93 (s, 2H), 3.04-2.81 (m, 2H), 2.42 (s, 3H), 2.40 (s, 3H), 2.04-1.85 (m, 4H), 1.84-1.75 (m, 2H), 1.66-1.50 (m, 2H). MS m/z: 511.15 [M+H]⁺.

### 1-{[3-(2,2-Difluoroethoxy)-2-pyrazinyl]methyl}-7-methyl-3-[(1r,4r)-4-(2-fluoro-6-tolyl)cyclohexyl]-1,8-diaza-2(1H)-naphthalenone (263)

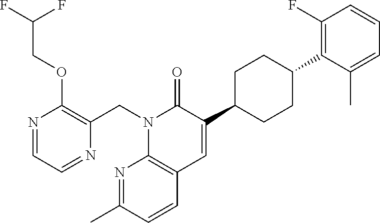 [SMILES: Cc1ccc2cc([C@H]3CC[C@H](c4c(C)cccc4F)CC3)c(=O)n(Cc3nccnc3OCC(F)F)c2n1]

Step 1: (3-(2,2-difluoroethoxy)pyrazin-2-yl)methanol: Followed the general procedure AK using (3-chloropyrazin-2-yl)methanol (600 mg, 4.15 mmol, 1 equiv.) and 2,2-difluoroethan-1-ol (10 mL) as the starting materials to give (3-(2,2-difluoroethoxy)pyrazin-2-yl)methanol (510 mg, 64%) as a light brown oil. MS m/z: 191 [M+H]⁺.

Step 2: (3-(2,2-difluoroethoxy)pyrazin-2-yl)methyl methanesulfonate: Followed the general procedure W using (3-(2,2-difluoroethoxy)pyrazin-2-yl)methanol (200 mg, 1.05 mmol, 1 equiv) and methanesulfonic anhydride (274 mg, 1.57 mmol, 1.5 equiv.) as the starting materials to give (3-(2,2-difluoroethoxy)pyrazin-2-yl)methyl methanesulfonate (200 mg, 70%) as a light brown oil. MS m/z: 269 [M+H]⁺.

Step 3: 1-((3-(2,2-difluoroethoxy)pyrazin-2-yl)methyl)-3-((1r,4r)-4-(2-fluoro-6-methylphenyl)cyclohexyl)-7-methyl-1,8-naphthyridin-2(1H)-one: Followed the general Procedure E using 3-((1r,4r)-4-(2-fluoro-6-methylphenyl)cyclohexyl)-7-methyl-1,8-naphthyridin-2(1H)-one (70 mg, 0.20 mmol, 1.0 equiv.) and (3-(2,2-difluoroethoxy)pyrazin-2-yl)methyl methanesulfonate (107 mg, 0.40 mmol, 2.0 equiv.) as the starting materials to give 1-((3-(2,2-difluoroethoxy)pyrazin-2-yl)methyl)-3-((1r,4r)-4-(2-fluoro-6-methylphenyl)cyclohexyl)-7-methyl-1,8-naphthyridin-2(1H)-one (67.8 mg, 63.6%) as a white solid. ¹H NMR (400 MHz, Chloroform-d) δ 7.96 (d, J=2.7 Hz, 1H), 7.90 (d, J=2.8 Hz, 1H), 7.75 (d, J=7.8 Hz, 1H), 7.53 (s, 1H), 7.05-6.98 (m, 2H), 6.95-6.79 (m, 2H), 6.33-6.15 (m, 1H), 5.97 (s, 2H), 4.70-4.59 (m, 2H), 3.09 (t, J=12.6, 1H), 2.89 (t, J=12.6 Hz, 1H), 2.50 (s, 3H), 2.37 (s, 3H), 2.22-2.08 (m, 4H), 1.88-1.79 (m, 2H), 1.54-1.46 (m, 2H). MS m/z: 523.20 [M+H]⁺.

### 7-Methyl-3-[(1r,4r)-4-(2-fluoro-6-tolyl)cyclohexyl]-1-{[3-(2,2,2-trifluoroethoxy)-2-pyrazinyl]methyl}-1,8-diaza-2(1H)-naphthalenone (264)

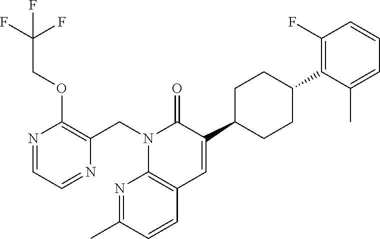 [SMILES: Cc1ccc2cc([C@H]3CC[C@H](c4c(C)cccc4F)CC3)c(=O)n(Cc3nccnc3OCC(F)(F)F)c2n1]

Step 1: (3-(2,2,2-trifluoroethoxy)pyrazin-2-yl)methanol: Followed the general procedure AK using (3-chloropyrazin-2-yl)methanol (600 mg, 4.151 mmol, 1 equiv.) and 2,2,2-trifluoroethan-1-ol (10 mL) as the starting materials to give (3-(2,2,2-trifluoroethoxy)pyrazin-2-yl)methanol (550 mg, 63%) as a light brown oil. MS m/z: 209 [M+H]⁺.

Step 2: (3-(2,2,2-trifluoroethoxy)pyrazin-2-yl)methyl methanesulfonate: Followed the general procedure W using (3-(2,2-difluoroethoxy)pyrazin-2-yl)methanol (200 mg, 0.048 mmol, 1.0 equiv.) and methanesulfonic anhydride (334 mg, 0.096 mmol, 2.0 equiv.) as the starting materials to give (3-(2,2-difluoroethoxy)pyrazin-2-yl)methyl methanesulfonate (150 mg, 54%) as a light brown oil. MS m/z: 287 [M+H]⁺.

Step 3: 3-((1r,4r)-4-(2-fluoro-6-methylphenyl)cyclohexyl)-7-methyl-1-((3-(2,2,2-trifluoroethoxy)pyrazin-2-yl)methyl)-1,8-naphthyridin-2(1H)-one: Followed the general Procedure E using (3-(2,2,2-trifluoroethoxy)pyrazin-2-yl)methyl methanesulfonate (150 mg, 0.524 mmol, 2 equiv.) and 3-((1r,4r)-4-(2-fluoro-6-methylphenyl)cyclohexyl)-7-methyl-1,8-naphthyridin-2(1H)-one (92 mg, 0.262 mmol, 1 equiv.) as the starting materials to give 3-((1r,4r)-4-(2-fluoro-6-methylphenyl)cyclohexyl)-7-methyl-1-((3-(2,2,2-trifluoroethoxy)pyrazin-2-yl)methyl)-1,8-naphthyridin-2(1H)-one (98.3 mg, 69.2%) as a white solid. ¹H NMR (400 MHz, Chloroform-d) δ 7.99 (d, J=2.8 Hz, 1H), 7.92 (d, J=2.8 Hz, 1H), 7.77 (d, J=7.8 Hz, 1H), 7.53 (d, J=0.8 Hz, 1H), 7.07-7.00 (m, 2H), 6.94-6.80 (m, 2H), 6.02 (s, 2H), 4.90-4.80 (m, 2H), 3.09 (t, J=12.6, 1H), 2.89 (t, J=12.6 Hz, 1H), 2.53 (s, 3H), 2.37 (s, 3H), 2.16 (q, J=12.8 Hz, 4H), 1.85 (s, 2H), 1.55-1.49 (m, 2H). MS m/z: 541.20 [M+H]⁺.

### 7-Cyclohexyl-3-methyl-5-{[3-(trifluoromethyl)-2-pyridyl]methyl}-1,4,5-triaza-6(5H)-naphthalenone (265)

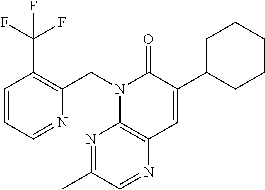 [SMILES: Cc1cnc2cc(C3CCCCC3)c(=O)n(Cc3ncccc3C(F)(F)F)c2n1]

Step 1: 7-cyclohexyl-3-methylpyrido[2,3-b]pyrazin-6(5H)-one: Followed the general procedure D using 3-amino-5-methylpyrazine-2-carbaldehyde (50 mg, 0.365 mmol, 1 equiv) and ethyl 2-cyclohexylacetate (62.07 mg, 0.365 mmol, 1 equiv) as the starting materials to give the product 7-cyclohexyl-3-methylpyrido[2,3-b]pyrazin-6(5H)-one (30 mg, 33%) as a yellow solid. MS m/z: 244 [M+H]⁺.

Step 2: 7-cyclohexyl-3-methyl-5-((3-(trifluoromethyl)pyridin-2-yl)methyl)pyrido[2,3-b]pyrazin-6(5H)-one: Followed the general procedure AP using 7-cyclohexyl-3-methylpyrido[2,3-b]pyrazin-6(5H)-one (30 mg, 0.123 mmol, 1 equiv) and 2-(chloromethyl)-3-(trifluoromethyl)pyridine (28.6 mg, 0.123 mmol, 1 equiv) as the starting materials to give 7-cyclohexyl-3-methyl-5-((3-(trifluoromethyl)pyridin-2-yl)methyl)pyrido[2,3-b]pyrazin-6(5H)-one (9.7 mg, 19.6%) as a white solid. ¹H NMR (400 MHz, Chloroform-d) δ 8.45-8.39 (m, 1H), 8.30 (s, 1H), 7.99-7.92 (m, 1H), 7.75 (d, J=0.9 Hz, 1H), 7.26-7.18 (m, 1H), 6.01 (s, 2H), 3.04-2.93 (m, 1H), 2.50 (s, 3H), 2.00 (d, J=12.2 Hz, 2H), 1.89-1.80 (m, 2H), 1.77 (d, J=13.0 Hz, 1H), 1.47-1.42 (m, 1H), 1.41-1.33 (m, 2H), 1.33-1.23 (m, 2H). MS m/z: 403.05 [M+H]⁺.

### 3-Methyl-7-[(1r,4r)-4-(5-fluoro-1-methyl-1H-1,2,7-triazainden-4-yl)cyclohexyl]-5-{[3-(trifluoromethyl)-2-pyridyl]methyl}-1,4,5-triaza-6(5H)-naphthalenone (266)

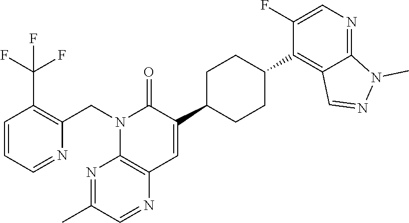 [SMILES: Cc1cnc2cc([C@H]3CC[C@H](c4c(F)cnc5c4cnn5C)CC3)c(=O)n(Cc3ncccc3C(F)(F)F)c2n1]

Step 1: 5-fluoro-2-hydrazineylpyrimidine: To a stirred mixture of 2-chloro-5-fluoropyrimidine (3 g, 22.6 mmol, 1 equiv.) in EtOH (30 mL) was added diimine hydrate hydrogen (3.33 g, 67.9 mmol, 3 equiv.). The resulting mixture was stirred for overnight at room temperature. The resulting mixture was diluted with water (200 mL), extracted with EtOAc (3×200 mL). The combined organic layers were washed by brine (100 mL), dried over anhydrous Na₂SO₄. The residue was purified by silica gel column chromatography, eluted with PE/EA (5:1) to afford 5-fluoro-2-hydrazineylpyrimidine (1.5 g, 37%) as a white solid. MS m/z: 129 [M+H]⁺.

Step 2: 3-iodopropiolaldehyde: To a stirred mixture of 3-(trimethylsilyl)propiolaldehyde (3 g, 23.7 mmol, 1 equiv.) and AgF (3.32 g, 26.1 mmol, 1.10 equiv.) in ACN (40 mL) was added NIS (5.88 g, 26.1 mmol, 1.10 equiv.). The resulting mixture was stirred for overnight at room temperature. The resulting mixture was diluted with water (200 mL), extracted with EtOAc (3×200 mL). The combined organic layers were washed by brine (100 mL), dried over anhydrous Na₂SO₄. The residue was purified by silica gel column chromatography, eluted with PE/EA (5:1) to afford 3-iodopropiolaldehyde (2.1 g, 44%) as a colorless oil. MS m/z: 181 [M+H]⁺.

Step 3: (E)-5-fluoro-2-(2-(3-iodoprop-2-yn-1-ylidene)hydrazineyl)pyrimidine: To a stirred mixture of 3-iodopropiolaldehyde (2.1 g, 11.6 mmol, 1 equiv.) and 5-fluoro-2-hydrazinylpyrimidine (1.50 g, 11.6 mmol, 1 equiv.) in THF (20 mL) was added TFA (2.66 g, 23.3 mmol, 2 equiv.). The resulting mixture was stirred for 2 h at 60° C. The resulting mixture was diluted with water (200 mL), extracted with EtOAc (3×100 mL). The combined organic layers were washed by brine (100 mL), dried over anhydrous Na₂SO₄. The residue was purified by silica gel column chromatography, eluted with PE/EA (3:1) to afford (E)-5-fluoro-2-(2-(3-iodoprop-2-yn-1-ylidene)hydrazineyl)pyrimidine (1 g, 26%) as a brown solid. MS m/z: 291 [M+H]⁺.

Step 4: 5-fluoro-4-iodo-1H-pyrazolo[3,4-b]pyridine: To a stirred mixture of (E)-5-fluoro-2-(2-(3-iodoprop-2-yn-1-ylidene)hydrazineyl)pyrimidine (1 g, 3.44 mmol, 1 equiv.) and 3-pentanone (0.89 g, 10.3 mmol, 3 equiv.) in THF (10 mL) was added TFAA (1.09 g, 5.17 mmol, 1.5 equiv.). The resulting mixture was stirred for 2 h at 60° C. The resulting mixture was diluted with water (100 mL), extracted with EtOAc (3×100 mL). The combined organic layers were washed by brine (100 mL), dried over anhydrous Na₂SO₄. The residue was purified by silica gel column chromatography, eluted with PE/EA (1:1) to afford 5-fluoro-4-iodo-1H-pyrazolo[3,4-b]pyridine (300 mg, 24%) as a white solid. MS m/z: 264 [M+H]⁺.

Step 5: 5-fluoro-4-iodo-1-methyl-1H-pyrazolo[3,4-b]pyridine: Followed the general procedure E using 5-fluoro-4-iodo-1H-pyrazolo[3,4-b]pyridine (280 mg, 1.06 mmol, 1 equiv.) and methyl iodide (453 mg, 3.19 mmol, 3 equiv.) as the starting materials to give 5-fluoro-4-iodo-1-methyl-1H-pyrazolo[3,4-b]pyridine (140 mg, 44%) as a white solid. MS m/z: 278 [M+H]⁺.

Step 6: ethyl 2-(4-(5-fluoro-1-methyl-1H-pyrazolo[3,4-b]pyridin-4-yl)cyclohex-3-en-1-yl)acetate: Followed the general procedure AL using 5-fluoro-4-iodo-1-methyl-1H-pyrazolo[3,4-b]pyridine (120 mg, 0.433 mmol, 1 equiv.) and ethyl 2-[4-(4,4,5,5-tetramethyl-1,3,2-dioxaborolan-2-yl)cyclohex-3-en-1-yl]acetate (191 mg, 0.64 mmol, 1.5 equiv.) as the starting materials to give ethyl 2-(4-(5-fluoro-i-methyl-1H-pyrazolo[3,4-b]pyridin-4-yl)cyclohex-3-en-1-yl)acetate (90 mg, 59%) as a white solid. MS m/z: 318 [M+H]⁺.

Step 7: ethyl 2-(4-(5-fluoro-i-methyl-1H-pyrazolo[3,4-b]pyridin-4-yl)cyclohexyl)acetate: Followed the general procedure AR using ethyl 2-(4-(5-fluoro-1-methyl-1H-pyrazolo[3,4-b]pyridin-4-yl)cyclohex-3-en-1-yl)acetate (80 mg, 0.252 mmol, 1 equiv.) as the starting material to give the crude product ethyl 2-(4-(5-fluoro-1-methyl-1H-pyrazolo[3,4-b]pyridin-4-yl)cyclohexyl)acetate (50 mg, crude) as a white solid. MS m/z: 320 [M+H]⁺.

Step 8: 7-((1r,4r)-4-(5-fluoro-i-methyl-1H-pyrazolo[3,4-b]pyridin-4-yl)cyclohexyl)-3-methylpyrido[2,3-b]pyrazin-6(5H)-one: Followed the general procedure D using ethyl 2-(4-(5-fluoro-1-methyl-1H-pyrazolo[3,4-b]pyridin-4-yl)cyclohexyl)acetate (50 mg, 0.157 mmol, 1 equiv.) and 3-amino-5-methylpyrazine-2-carbaldehyde (27.9 mg, 0.204 mmol, 1.3 equiv.) as the starting materials to give 7-((1r,4r)-4-(5-fluoro-1-methyl-1H-pyrazolo[3,4-b]pyridin-4-yl)cyclohexyl)-3-methylpyrido[2,3-b]pyrazin-6(5H)-one (40 mg, 29%) as a white solid. MS m/z: 393 [M+H]⁺.

Step 9: 7-((1r,4r)-4-(5-fluoro-1-methyl-1H-pyrazolo[3,4-b]pyridin-4-yl)cyclohexyl)-3-methyl-5-((3-(trifluoromethyl)pyridin-2-yl)methyl)pyrido[2,3-b]pyrazin-6(5H)-one: Followed the general procedure E using 7-((1r,4r)-4-(5-fluoro-1-methyl-1H-pyrazolo[3,4-b]pyridin-4-yl)cyclohexyl)-3-methylpyrido[2,3-b]pyrazin-6(5H)-one (30 mg, 0.076 mmol, 1 equiv.) and 2-(chloromethyl)-3-(trifluoromethyl)pyridine (19.4 mg, 0.099 mmol, 1.3 equiv.) as the starting materials to give 7-((1r,4r)-4-(5-fluoro-1-methyl-1H-pyrazolo[3,4-b]pyridin-4-yl)cyclohexyl)-3-methyl-5-((3-(trifluoromethyl)pyridin-2-yl)methyl)pyrido[2,3-b]pyrazin-6(5H)-one (5.2 mg, 12%) as a white solid. ¹H NMR (400 MHz, Chloroform-d) δ 8.49-8.41 (m, 1H), 8.41-8.30 (m, 2H), 8.11 (s, 1H), 7.99 (dd, J=7.7, 1.5 Hz, 1H), 7.85 (d, J=0.9 Hz, 1H), 7.24 (s, 1H), 6.05 (s, 2H), 4.14 (s, 3H), 3.36-3.17 (m, 2H), 2.53 (s, 3H), 2.24 (d, J=12.8 Hz, 2H), 2.19-2.03 (m, 4H), 1.67-1.63 (m, 2H). MS m/z: 552.20 [M+H]⁺.

### 3-(1,1-Difluoroethyl)-7-[(1r,4r)-4-(2-fluoro-6-tolyl)cyclohexyl]-5-{[3-(trifluoromethyl)-2-pyridyl]methyl}-1,4,5-triaza-6(5H)-naphthalenone (267)

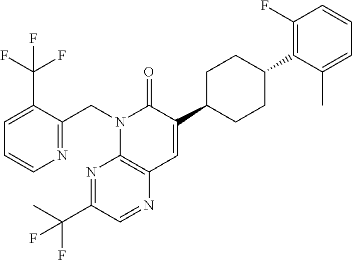 [SMILES: Cc1cccc(F)c1[C@H]1CC[C@H](c2cc3ncc(C(C)(F)F)nc3n(Cc3ncccc3C(F)(F)F)c2=O)CC1]

Step 1: 3-(1-ethoxyvinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: Followed the general procedure V using 3-chloro-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (50 mg, 0.094 mmol, 1 equiv.) and tributyl(1-ethoxyvinyl)stannane (102 mg, 0.282 mmol, 3 equiv.) as the starting materials to give 3-(1-ethoxyvinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (30 mg, 56%) as a white solid. MS m/z: 567 [M+H]⁺.

Step 2: 3-acetyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: To a stirred solution of 3-(1-ethoxyvinyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (30 mg, 0.053 mmol, 1 equiv) in dioxane (1 mL) was added aq.HCl (2M, 1.5 mL) at room temperature. The resulting mixture was stirred for 1 h at room temperature. Desired product could be detected by LCMS. After removing the solvent, the mixture was basified to pH 8 with saturated NaHCO₃ (aq.). The resulting mixture was extracted with EtOAc (3×30 mL). The combined organic layers were washed with brine (30 mL), dried over anhydrous Na₂SO₄. After filtration, the filtrate was concentrated under reduced pressure. The crude product 3-acetyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (20 mg) was used for next step without further purification. MS m/z: 539 [M+H]⁺.

Step 3: 3-(1,1-difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one: A solution of 3-acetyl-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (20 mg, 0.037 mmol, 1 equiv.) in BAST (1 mL) was stirred for overnight at room temperature. Desired product could be detected by LCMS. The reaction was quenched by the addition of sat. NH₄Cl (aq.) (5 mL) at 0° C. The resulting mixture was extracted with EtOAc (3×20 mL). The combined organic layers were washed with brine (2×10 mL), dried over anhydrous Na₂SO₄. After filtration, the filtrate was concentrated under reduced pressure. The residue was purified by Prep-TLC (PE/EA 5:1) to afford 3-(1,1-difluoroethyl)-7-((1r,4r)-4-(2-fluoro-6-methylphenyl)cyclohexyl)-5-((3-(trifluoromethyl)pyridin-2-yl)methyl)pyrido[2,3-b]pyrazin-6(5H)-one (6.2 mg, 29.4%) as a white solid. ¹H NMR (300 MHz, Chloroform-d) δ 8.77 (s, 1H), 8.42 (d, J=4.9 Hz, 1H), 7.99 (d, J=7.9 Hz, 1H), 7.87 (s, 1H), 7.23 (s, 1H), 7.11-6.98 (m, 1H), 6.95-6.80 (m, 2H), 6.01 (s, 2H), 3.20 (t, J=12.2 Hz, 1H), 2.92 (t, J=12.2 Hz, 1H), 2.39 (s, 3H), 2.19 (d, J=12.1 Hz, 4H), 1.91-1.73 (m, 5H), 1.56 (d, J=12.4 Hz, 2H). MS m/z: 561.15 [M+H]⁺.

### 1-{[3-(2,2-Difluoroethyl)-2-pyridyl]methyl}-7-methyl-3-[(1r,4r)-4-(2-fluoro-6-tolyl)cyclohexyl]-1,8-diaza-2(1H)-naphthalenone (268)

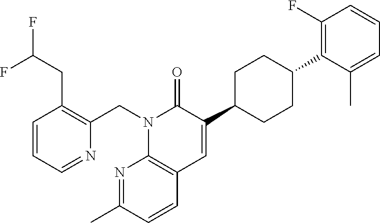 [SMILES: Cc1ccc2cc([C@H]3CC[C@H](c4c(C)cccc4F)CC3)c(=O)n(Cc3ncccc3CC(F)F)c2n1]

Step 1: 1-((3-(2,2-difluoroethyl)pyridin-2-yl)methyl)-3-((1r,4r)-4-(2-fluoro-6-methylphenyl)cyclohexyl)-7-methyl-1,8-naphthyridin-2(1H)-one: Followed the general procedure AP using 3-((1r,4r)-4-(2-fluoro-6-methylphenyl)cyclohexyl)-7-methyl-1,8-naphthyridin-2(1H)-one (50 mg, 0.143 mmol, 1.0 equiv.) and 2-(chloromethyl)-3-(2,2-difluoroethyl)pyridine (32.8 mg, 0.172 mmol, 1.2 equiv.) as the starting materials to give 1-((3-(2,2-difluoroethyl)pyridin-2-yl)methyl)-3-((1r,4r)-4-(2-fluoro-6-methylphenyl)cyclohexyl)-7-methyl-1,8-naphthyridin-2(1H)-one (27.9 mg, 37.1%) as a white solid. ¹H NMR (400 MHz, Chloroform-d) δ 8.44 (s, 11H), 7.74 (d, =7.8 Hz, 2H), 7.53 (s, 1H), 7.24 (s, 1H), 7.07-6.97 (m, 2H), 6.95-6.80 (m, 2H), 6.36-5.94 (m, 3H), 3.73-3.27 (m, 2H), 3.11 (t, J=12.6 Hz, 1H), 2.89 (t, J=12.6 Hz, 1H), 2.52 (s, 311), 2.37 (s, 311), 2.25-2.08 (m, 4H), 1.83 (d, J=12.7 Hz, 2H), 1.58-1.44 (m, 2H). MS m/z: 506.20 [M+H]⁺.

### 1-{[3-(1,1-Difluoroethyl)-2-pyrazinyl]methyl}-7-methyl-3-[(1r,4r)-4-(2-fluoro-6-tolyl)cyclohexyl]-1,8-diaza-2(1H)-naphthalenone (269)

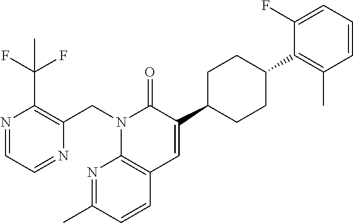 [SMILES: Cc1ccc2cc([C@H]3CC[C@H](c4c(C)cccc4F)CC3)c(=O)n(Cc3nccnc3C(C)(F)F)c2n1]

Step 1: 2-chloro-3-(1,1-difluoroethyl)pyrazine: Followed the general Procedure AE using 1-(3-chloropyrazin-2-yl)ethan-1-one (500 mg, 3.19 mmol, 1.00 equiv.) and DAST (2.58 g, 15.9 mmol, 5.00 equiv.) as the starting materials to give 2-chloro-3-(1,1-difluoroethyl)pyrazine (250 mg, 44%) as a yellow oil. MS m/z: 179 [M+H]⁺.

Step 2: (3-(1,1-difluoroethyl)pyrazin-2-yl)methanol: Followed the general Procedure V using 2-chloro-3-(1,1-difluoroethyl)pyrazine (250 mg, 1.4 mmol, 1.0 equiv.) and (tributylstannyl)methanol (678 mg, 2.1 mmol, 1.5 equiv.) as the starting materials to give (3-(1,1-difluoroethyl)pyrazin-2-yl)methanol (60 mg, 25%) as a yellow oil. MS m/z: 175 [M+H]⁺.

Step 3: 2-(chloromethyl)-3-(1,1-difluoroethyl)pyrazine: Followed the general Procedure J using (3-(1,1-difluoroethyl)pyrazin-2-yl)methanol (60 mg, 0.343 mmol, 1.00 equiv) as the starting materials to give the crude product 2-(chloromethyl)-3-(1,1-difluoroethyl)pyrazine (40 mg, crude) as a yellow solid. MS m/z: 193 [M+H]⁺.

Step 4: 1-((3-(1,1-difluoroethyl)pyrazin-2-yl)methyl)-3-((1r,4r)-4-(2-fluoro-6-methylphenyl)cyclohexyl)-7-methyl-1,8-naphthyridin-2(1H)-one: Followed the general Procedure AP using 2-(chloromethyl)-3-(1,1-difluoroethyl)pyrazine (40 mg, 0.207 mmol, 1.10 equiv.) and 3-((1r,4r)-4-(2-fluoro-6-methylphenyl)cyclohexyl)-7-methyl-1,8-naphthyridin-2(1H)-one (66.1 mg, 0.188 mmol, 1.00 equiv.) as the starting materials to give 1-((3-(1,1-difluoroethyl)pyrazin-2-yl)methyl)-3-((1r,4r)-4-(2-fluoro-6-methylphenyl)cyclohexyl)-7-methyl-1,8-naphthyridin-2(1H)-one (11.6 mg, 12.1%) as an off-white solid. ¹H NMR (400 MHz, Chloroform-d) δ 8.38-8.30 (m, 2H), 7.75 (d, J=7.8 Hz, 1H), 7.54 (s, 1H), 7.02 (dt, J=13.6, 7.8 Hz, 2H), 6.94-6.81 (m, 2H), 6.20 (s, 2H), 3.11 (t, J=12.3, 1H), 2.90 (t, J=12.3 Hz, 1H), 2.47 (s, 3H), 2.37 (s, 3H), 2.28-2.10 (m, 7H), 1.87-1.79 (m, 2H), 1.53 (s, 2H). MS m/z: 507.20 [M+H]⁺.

### Biological Assay Data and Procedures

Exemplary compounds were evaluated for classical GPCR β-arrestin recruitment via complement C5a receptor 1 (C5aR1) activation in live CHO-K1 cells. This cell line expresses two fragments of the P-galactosidase protein coupled to β-arrestin and C5aR1. Complementation of the two fragments allows for the assessment of both C5aR1 agonism and antagonism. Recombinant hC5a (EC₉₀) was used as the competing natural agonist against disclosed compounds (more information on the expanded protocol and cell line can be found on the manufacturer's website: PathHunter® CHO-K1 C5aR1 β-arrestin cell line, DiscoverX, Catalog No. 93-0557C2). The results in Table 1 demonstrate that compounds of the disclosure are potent antagonists of C5aR1. IC₅₀ ranges: A: IC₅₀≤500 nM; B: 500 nM<IC₅₀≤5 uM; C: 5 uM<IC₅₀≤100 uM; D: IC₅₀>100 uM.

TABLE 1
In vitro cellular IC₅₀ values for exemplary compounds

| | [Antagonist] | [Agonist] |
|---|---|---|
| | B-arrestin2 | B-arrestin2 |
| | recruitment: | recruitment: |
| | Average | Average |
| No. | IC50 (nM) | EC50 (nM) |
| 1 | A | NA |
| 2 | B | Non-Active |
| 3 | B | Non-Active |
| 4 | B | Non-Active |
| 5 | B | Non-Active |
| 6 | A | Non-Active |
| 7 | A | Non-Active |
| 8 | C | Non-Active |
| 9 | C | Non-Active |
| 10 | A | Non-Active |
| 11 | D | Non-Active |
| 12 | A | Non-Active |
| 13 | A | Non-Active |
| 14 | D | Non-Active |
| 15 | D | Non-Active |
| 16 | A | Non-Active |
| 17 | B | Non-Active |
| 18 | D | Non-Active |
| 20 | B | Non-Active |
| 21 | A | Non-Active |
| 22 | B | Non-Active |
| 23 | B | Non-Active |
| 24 | B | Non-Active |
| 25 | A | Non-Active |
| 26 | B | Non-Active |
| 27 | D | Non-Active |
| 28 | C | Non-Active |
| 29 | C | Non-Active |
| 30 | C | Non-Active |
| 31 | A | Non-Active |
| 32 | A | Non-Active |
| 33 | B | Non-Active |
| 34 | D | Non-Active |
| 35 | A | Non-Active |
| 36 | B | Non-Active |
| 37 | C | Non-Active |
| 38 | A | Non-Active |
| 39 | B | Non-Active |
| 40a | C | Non-Active |
| 40b | D | Non-Active |
| 41 | A | Non-Active |
| 42 | B | Non-Active |
| 43a | B | Non-Active |
| 43b | B | Non-Active |
| 44 | D | Non-Active |
| 45 | D | Non-Active |
| 46 | B | Non-Active |
| 47 | B | Non-Active |
| 48 | B | Non-Active |
| 49a | C | Non-Active |
| 49b | B | Non-Active |
| 50 | B | Non-Active |
| 51 | B | Non-Active |
| 52 | D | Non-Active |
| 53a | C | Non-Active |
| 53b | B | Non-Active |
| 54 | D | Non-Active |
| 55 | B | Non-Active |
| 56 | C | Non-Active |
| 57 | A | Non-Active |
| 58 | B | Non-Active |
| 59 | D | Non-Active |
| 60 | A | Non-Active |
| 61a | B | Non-Active |
| 61b | A | Non-Active |
| 62 | B | Non-Active |
| 63 | B | Non-Active |
| 64 | B | Non-Active |
| 65 | B | Non-Active |
| 66 | A | Non-Active |
| 67 | A | Non-Active |
| 68 | A | Non-Active |
| 69 | D | Non-Active |
| 70 | B | Non-Active |
| 71 | B | Non-Active |
| 72 | D | Non-Active |
| 73 | B | Non-Active |
| 74 | B | — |
| 75 | B | — |
| 76 | B | — |
| 77 | B | — |
| 78 | B | — |
| 79 | B | — |
| 80 | B | — |
| 81 | A | — |
| 82 | D | — |
| 83 | A | — |
| 84 | D | — |
| 85 | A | — |
| 86 | B | Non-Active |
| 87 | A | Non-Active |
| 88 | A | Non-Active |
| 89 | C | Non-Active |
| 90 | C | — |
| 91 | B | — |
| 92 | A | — |
| 93 | D | — |
| 94 | A | — |
| 95 | B | — |
| 96 | A | — |
| 97 | B | — |
| 98 | A | — |
| 99a | B | — |
| 99b | A | — |
| 100 | A | — |
| 101 | B | — |
| 102 | A | — |
| 103 | B | — |
| 104 | B | — |
| 105 | A | — |
| 106 | A | — |
| 107 | B | — |
| 108 | B | — |
| 109 | B | — |
| 110 | A | — |
| 111 | C | — |
| 112 | C | — |
| 113 | D | — |
| 114 | C | — |
| 115 | B | — |
| 116 | B | — |
| 117 | B | — |
| 118 | B | Non-Active |
| 119 | B | — |
| 120 | A | Non-Active |
| 121a | A | — |
| 121b | B | — |
| 122 | A | — |
| 123 | A | Non-Active |
| 124 | A | Non-Active |
| 125 | B | Non-Active |
| 126 | B | Non-Active |
| 127 | A | Non-Active |
| 128 | C | Non-Active at 10 μM |
| 129 | A | Non-Active |
| 130 | A | Non-Active |
| 131 | A | Non-Active |
| 132 | D | Non-Active |
| 133 | A | Non-Active |
| 134 | A | Non-Active at 10 μM |
| 135A | C | Non-Active at 10 μM |
| 135B | C | Non-Active at 10 μM |
| 136 | D | Non-Active |
| 137A | C | Non-Active |
| 137B | A | Non-Active at 10 μM |
| 138 | A | Non-Active |
| 139 | D | Non-Active at 10 μM |
| 140 | A | Non-Active at 10 μM |
| 141 | A | Non-Active |
| 142A | D | Non-Active |
| 142B | — | Non-Active |
| 143A | A | Non-Active |
| 143B | B | Non-Active |
| 144A | D | Non-Active at 10 μM |
| 144B | D | Non-Active at 10 μM |
| 145 | A | Non-Active |
| 146A | C | Non-Active at 10 μM |
| 146B | B | Non-Active at 10 μM |
| 147A | A | Non-Active at 10 μM |
| 147B | A | Non-Active |
| 148A | B | Non-Active |
| 148B | A | Non-Active |
| 149A | C | Non-Active |
| 149B | A | Non-Active |
| 150A | A | Non-Active at 10 μM |
| 150B | A | Non-Active |
| 151 | B | Non-Active at 10 μM |
| 152 | A | Non-Active |
| 153 | B | Non-Active at 10 μM |
| 154 | A | Non-Active at 10 μM |
| 155 | A | Non-Active at 10 μM |
| 156 | B | Non-Active at 10 μM |
| 157 | B | Non-Active at 10 μM |
| 158 | B | Non-Active at 10 μM |
| 159 | C | Non-Active at 10 μM |
| 160A | C | Non-Active at 10 μM |
| 160B | C | Non-Active at 10 μM |
| 161A | B | Non-Active at 10 μM |
| 161B | C | Non-Active at 10 μM |
| 162 | A | Non-Active |
| 163 | B | Non-Active at 10 μM |
| 164 | B | Non-Active at 10 μM |
| 165 | A | Non-Active at 10 μM |
| 166 | A | Non-Active at 10 μM |
| 167 | A | Non-Active at 10 μM |
| 168A | B | Non-Active at 10 μM |
| 168B | A | Non-Active at 10 μM |
| 169 | A | Non-Active at 10 μM |
| 170 | A | Non-Active at 10 μM |
| 171A | A | Non-Active at 10 μM |
| 171B | A | Non-Active at 10 μM |
| 171C | A | Non-Active |
| 171D | B | Non-Active at 10 μM |
| 172A | B | Non-Active at 10 μM |
| 172B | B | Non-Active at 10 μM |
| 172C | A | Non-Active at 10 μM |
| 172D | C | Non-Active at 10 μM |
| 173 | A | Non-Active at 10 μM |
| 174A | C | Non-Active at 10 μM |
| 174B | B | Non-Active at 10 μM |
| 175A | A | Non-Active |
| 175B | A | Non-Active at 10 μM |
| 175C | A | Non-Active |
| 175D | B | Non-Active at 10 μM |
| 176 | A | Non-Active |
| 177 | A | Non-Active |
| 178 | A | Non-Active |
| 179 | A | Non-Active at 10 μM |
| 180 | A | Non-Active at 10 μM |
| 181 | C | Non-Active at 10 μM |
| 182 | A | Non-Active at 10 μM |
| 183 | A | Non-Active at 10 μM |
| 184 | B | Non-Active at 10 μM |
| 185 | B | Non-Active at 10 μM |
| 186 | C | Non-Active at 10 μM |
| 187A | A | Non-Active at 10 μM |
| 187B | A | Non-Active at 10 μM |
| 187C | A | Non-Active at 10 μM |
| 187D | B | Non-Active at 10 μM |
| 188 | A | Non-Active at 10 μM |
| 189 | A | Non-Active at 10 μM |
| 190 | B | Non-Active at 10 μM |
| 191 | C | Non-Active at 10 μM |
| 192 | B | Non-Active at 10 μM |
| 193 | A | Non-Active |
| 194 | A | Non-Active |
| 195 | A | Non-Active |
| 196 | A | Non-Active |
| 197A | A | Non-Active at 10 μM |
| 197B | B | Non-Active at 10 μM |
| 197C | C | Non-Active at 10 μM |
| 197D | A | Non-Active |
| 198 | A | Non-Active |
| 199 | A | Non-Active |
| 200A | — | Non-Active at 10 μM |
| 200B | — | Non-Active at 10 μM |
| 200C | B | Non-Active at 10 μM |
| 200D | A | Non-Active at 10 μM |
| 201 | A | Non-Active at 10 μM |
| 202 | A | Non-Active at 10 μM |
| 203 | A | Non-Active at 10 μM |
| 204 | A | Non-Active at 10 μM |
| 205 | A | Non-Active at 10 μM |
| 206 | B | Non-Active at 10 μM |
| 207 | C | Non-Active at 10 μM |
| 208 | A | Non-Active at 10 μM |
| 209 | A | Non-Active at 10 μM |
| 210 | A | Non-Active at 10 μM |
| 211 | A | Non-Active at 10 μM |
| 212 | A | Non-Active at 10 μM |
| 213 | A | Non-Active at 10 μM |
| 214 | C | Non-Active at 10 μM |
| 215 | A | Non-Active at 10 μM |
| 216 | B | Non-Active at 10 μM |
| 217 | C | Non-Active at 10 μM |
| 218 | — | Non-Active at 10 μM |
| 219 | A | Non-Active at 10 μM |
| 220 | A | Non-Active at 10 μM |
| 221 | A | Non-Active at 10 μM |
| 222 | A | Non-Active at 10 μM |
| 223 | A | Non-Active at 10 μM |
| 224 | A | Non-Active at 10 μM |
| 225 | A | Non-Active at 10 μM |
| 226 | A | Non-Active at 10 μM |
| 227 | A | Non-Active at 10 μM |
| 228 | A | Non-Active at 10 μM |
| 229 | A | Non-Active at 10 μM |
| 230 | A | Non-Active at 10 μM |
| 231 | A | Non-Active at 10 μM |
| 232 | A | Non-Active at 10 μM |
| 233 | A | Non-Active at 10 μM |
| 234 | A | Non-Active at 10 μM |
| 235 | A | Non-Active at 10 μM |
| 236 | A | Non-Active at 10 μM |
| 237 | A | Non-Active |
| 238 | A | Non-Active |
| 239 | A | Non-Active |
| 240 | A | Non-Active |
| 241 | A | Non-Active |
| 242 | A | Non-Active |
| 243A | A | Non-Active |
| 243B | A | Non-Active |
| 244 | A | Non-Active |
| 245 | A | Non-Active at 10 μM |
| 246 | A | Non-Active at 10 μM |
| 247 | A | Non-Active at 10 μM |
| 248 | A | Non-Active at 10 μM |
| 249 | A | Non-Active at 10 μM |
| 250 | A | Non-Active at 10 μM |
| 251 | A | Non-Active at 10 μM |
| 252 | A | Non-Active at 10 μM |
| 253 | A | Non-Active |
| 254 | A | Non-Active |
| 255 | A | Non-Active |
| 256 | A | Non-Active |
| 257 | A | Non-Active |
| 258 | A | Non-Active |
| 259 | A | Non-Active |
| 260 | A | Non-Active |
| 261 | A | Non-Active |
| 262 | A | Non-Active |
| 263 | A | Non-Active |
| 264 | A | Non-Active |
| 265 | B | Non-Active |
| 266 | C | Non-Active |
| 267 | A | Non-Active |
| 268 | A | Non-Active |
| 269 | A | Non-Active |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 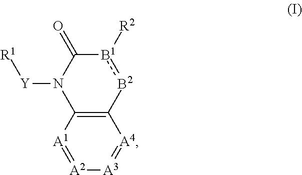
or a pharmaceutically acceptable salt thereof, wherein:
R¹ is an optionally substituted aryl, optionally substituted heteroaryl, optionally substituted carbocyclyl, or optionally substituted heterocyclyl;
R² is an optionally substituted carbocyclyl or optionally substituted heterocyclyl;
A¹, A², A³, and A⁴ are each independently ═C(R³)— or ═N—;
B¹ is —C(R⁴)ₜ— or —N—, wherein t is 0 or 1 as valency permits;
B² is ═CR⁴—, —N—, or —C(O)—;
Y is —C(R⁵)₂— or a bond;
each R³ is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted heteroaliphatic, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, or —S(O)₂R^{A};
each R⁴ is independently hydrogen, halogen, optionally substituted alkyl, optionally substituted alkenyl, optionally substituted alkynyl, —OR^{A}, —N(R^{A})₂, —SR^{A}, or —CN;
each R⁵ is independently hydrogen, halogen, or optionally substituted alkyl; and
each R^{A} is independently hydrogen, optionally substituted acyl, optionally substituted alkyl, optionally substituted alkenyl, optionally substituted alkynyl, optionally substituted heteroaliphatic, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom.

2. The compound of claim 1, or a pharmaceutically acceptable salt thereof, wherein Y is —C(R⁵)₂—.

3. The compound of claim 1 or 2, or a pharmaceutically acceptable salt thereof, wherein each R⁵ is independently hydrogen or optionally substituted alkyl.

4. The compound of any one of claims 1-3, or a pharmaceutically acceptable salt thereof, wherein Y is —CH₂—.

5. The compound of claim 1, or a pharmaceutically acceptable salt thereof, wherein Y is a bond.

6. The compound of any one of claims 1-5, or a pharmaceutically acceptable salt thereof, wherein B¹ is ═C—.

7. The compound of any one of claims 1-5, or a pharmaceutically acceptable salt thereof, wherein B¹ is —N—.

8. The compound of any one of claims 1-7, or a pharmaceutically acceptable salt thereof, wherein B² is —C(O)—.

9. The compound of any one of claims 1-7, or a pharmaceutically acceptable salt thereof, wherein B² is ═C(R⁴)—.

10. The compound of any one of claims 1-7, or a pharmaceutically acceptable salt thereof, wherein B² is ═C(H)— or ═C(CH₃)—.

11. The compound of any one of claims 1-9, or a pharmaceutically acceptable salt thereof, wherein each R⁴ is independently hydrogen or optionally substituted alkyl.

12. The compound of any one of claims 1-9, or a pharmaceutically acceptable salt thereof, wherein each R⁴ is hydrogen.

13. The compound of any one of claims 1-9, or a pharmaceutically acceptable salt thereof, wherein each R⁴ is optionally substituted alkyl.

14. The compound of any one of claims 1-13, or a pharmaceutically acceptable salt thereof, wherein A¹ is ═N—.

15. The compound of any one of claims 1-13, or a pharmaceutically acceptable salt thereof, wherein A¹ is ═C(R³)—.

16. The compound of any one of claims 1-15, or a pharmaceutically acceptable salt thereof, wherein A² is ═N—.

17. The compound of any one of claims 1-15, or a pharmaceutically acceptable salt thereof, wherein A² is ═C(R³)—.

18. The compound of any one of claims 1-17, or a pharmaceutically acceptable salt thereof, wherein A³ is ═N—.

19. The compound of any one of claims 1-17, or a pharmaceutically acceptable salt thereof, wherein A³ is ═C(R³)—.

20. The compound of any one of claims 1-19, or a pharmaceutically acceptable salt thereof, wherein A⁴ is ═N—.

21. The compound of any one of claims 1-19, or a pharmaceutically acceptable salt thereof, wherein A⁴ is ═C(R³)—.

22. The compound of any one of claims 1-21, or a pharmaceutically acceptable salt thereof, wherein no more than two of A¹, A², A³, and A⁴ are ═N—.

23. The compound of any one of claims 1-22, or a pharmaceutically acceptable salt thereof, wherein A¹ and A⁴ are ═N—; and A² and A³ are ═C(R³)—.

24. The compound of any one of claims 1-22, or a pharmaceutically acceptable salt thereof, wherein A¹ and A³ are ═N—; and A² and A⁴ are ═C(R³)—.

25. The compound of any one of claims 1-22, or a pharmaceutically acceptable salt thereof, wherein A² and A⁴ are ═N—; and A¹ and A³ are ═C(R³)—.

26. The compound of any one of claims 1-22, or a pharmaceutically acceptable salt thereof, wherein A¹, A², and A³ are ═C(R³)—; and A⁴ is ═N—.

27. The compound of any one of claims 1-22, or a pharmaceutically acceptable salt thereof, wherein A², A³, and A⁴ are ═C(R³)—; and A¹ is ═N—.

28. The compound of any one of claims 1-22, or a pharmaceutically acceptable salt thereof, wherein A¹, A², A³, and A⁴ are ═C(R³)—.

29. The compound of any one of claims 1-28, or a pharmaceutically acceptable salt thereof, wherein each R³ is independently hydrogen, optionally substituted alkyl, or —OR^{A}.

30. The compound of any one of claims 1-29, or a pharmaceutically acceptable salt thereof, wherein R¹ is optionally substituted aryl, optionally substituted heteroaryl, or optionally substituted carbocyclyl.

31. The compound of any one of claims 1-29, or a pharmaceutically acceptable salt thereof, wherein R¹ is optionally substituted aryl or optionally substituted heteroaryl.

32. The compound of any one of claims 1-29, or a pharmaceutically acceptable salt thereof, wherein R¹ is optionally substituted aryl.

33. The compound of any one of claims 1-29, or a pharmaceutically acceptable salt thereof, wherein R¹ is optionally substituted heteroaryl.

34. The compound of any one of claims 1-33, or a pharmaceutically acceptable salt thereof, wherein R¹ is 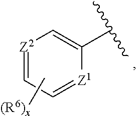
wherein Z¹ and Z² are each independently CH, CR⁶, or N; each R⁶ is independently halogen, optionally substituted alkyl, optionally substituted cycloalkyl, —SF₅, or —OR^{A}; and x is 0, 1, 2, 3, or 4.

35. The compound of any one of claims 1-33, or a pharmaceutically acceptable salt thereof, wherein R¹ is 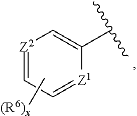
wherein Z¹ and Z² are each independently CH, CR⁶, or N; each R⁶ is independently halogen, optionally substituted alkyl, —SF₅, or —OR^{A}; and x is 0, 1, 2, 3, or 4.

36. The compound of any one of claims 1-33, or a pharmaceutically acceptable salt thereof, wherein R¹ is 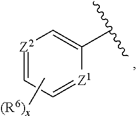
wherein Z¹ and Z² are each independently CH, CR⁶, or N; each R⁶ is independently —F, —SF₅, cyclopropyl, —CH₃, —CF₃, —CHF₂, —CF₂CH₃, —CH₂CF₃, —CH₂CHF₂, —O-cyclopropyl, —OCH₃, —OCH₂CH₃, —OCH₂CF₃, —OCH₂CF₃, —OCH₂CHF₂, —OCF₃, or —OCHF₂; and x is 0, 1, 2, 3, or 4.

37. The compound of any one of claims 1-35, or a pharmaceutically acceptable salt thereof, wherein R¹ is 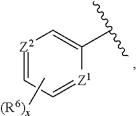
wherein Z¹ and Z² are each independently CH, CR⁶, or N; each R⁶ is independently —F, —SF₅, —CH₃, —CF₃, —CHF₂, —OCH₂CF₃, —OCH₃, —OCF₃, or —OCHF₂; and x is 0, 1, 2, 3, or 4.

38. The compound of any one of claims 1-35, or a pharmaceutically acceptable salt thereof, wherein R¹ is

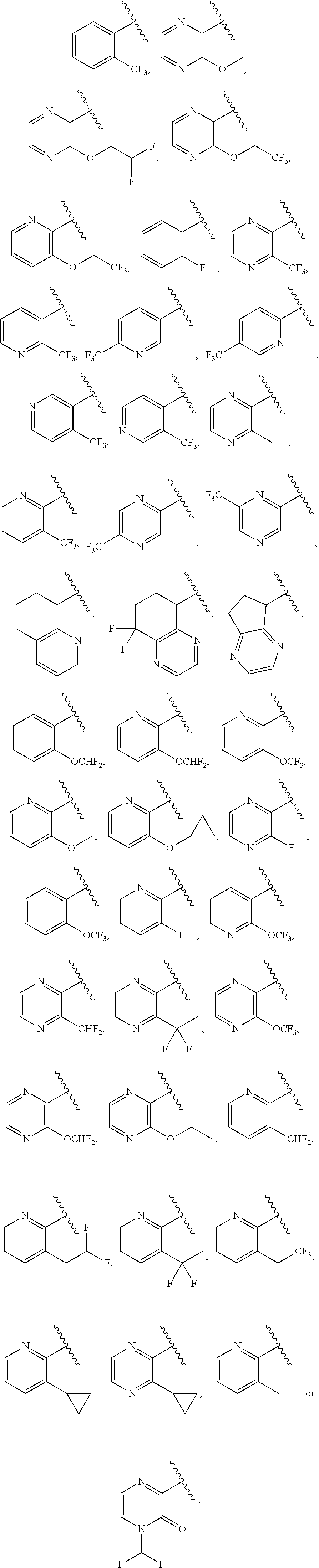

39. The compound of any one of claims 1-37, or a pharmaceutically acceptable salt thereof, wherein R¹ is

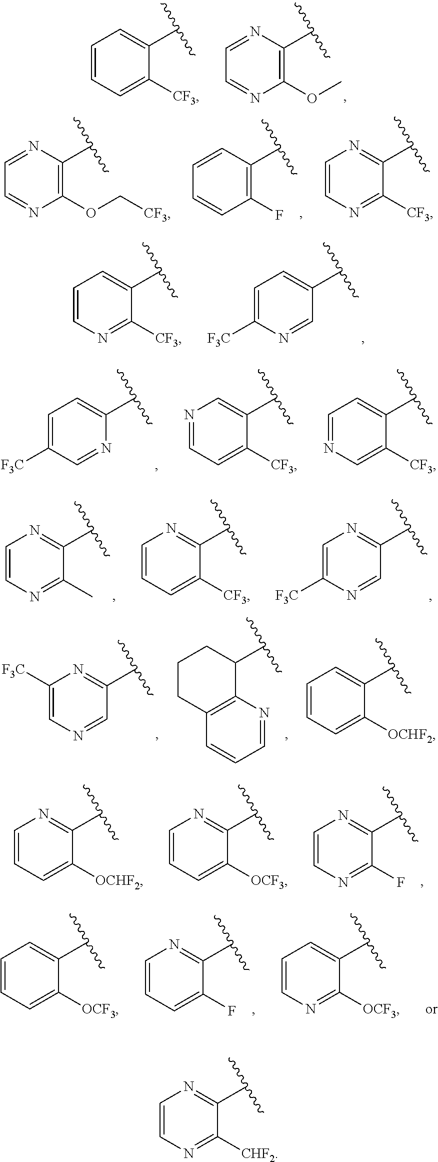

40. The compound of any one of claims 1-39, or a pharmaceutically acceptable salt thereof, wherein R² is optionally substituted carbocyclyl.

41. The compound of any one of claims 1-40, or a pharmaceutically acceptable salt thereof, wherein R² is optionally substituted cycloalkyl.

42. The compound of any one of claims 1-41, or a pharmaceutically acceptable salt thereof, wherein R² is optionally substituted C₃₋₈ cycloalkyl.

43. The compound of any one of claims 1-42, or a pharmaceutically acceptable salt thereof, wherein R² is optionally substituted cyclohexyl.

44. The compound of any one of claims 1-43, or a pharmaceutically acceptable salt thereof, wherein R² is 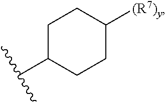
wherein each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

45. The compound of any one of claims 1-44, or a pharmaceutically acceptable salt thereof, wherein R² is 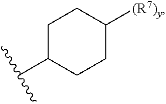
wherein each R⁷ is independently halogen, optionally substituted alkyl, or optionally substituted aryl, or two instances of R⁷ together with the atoms to which they are attached form a substituted or unsubstituted carbocyclyl or substituted or unsubstituted heterocyclyl; and y is 0, 1, or 2.

46. The compound of any one of claims 1-45, or a pharmaceutically acceptable salt thereof, wherein R² is

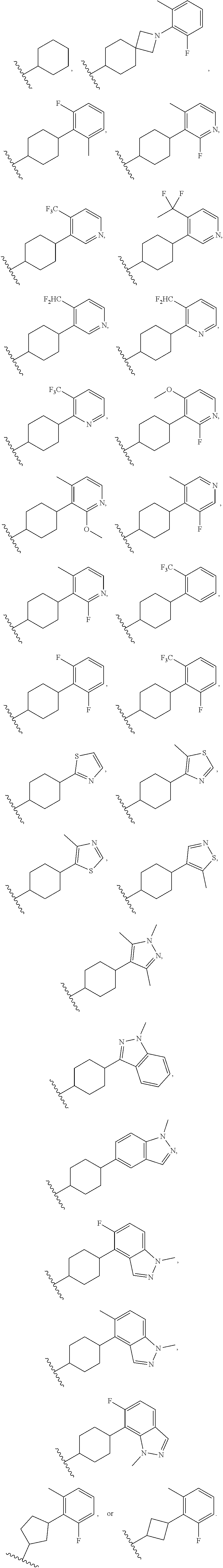

47. The compound of any one of claims 1-46, or a pharmaceutically acceptable salt thereof, wherein R² is

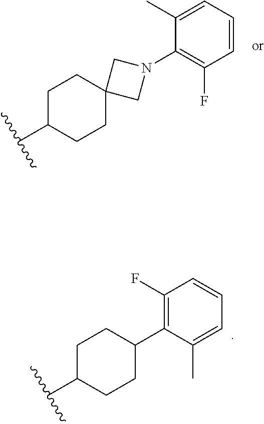 [SMILES: *C1CCC2(CC1)CN(c1c(C)cccc1F)C2 ; *C1CCC(c2c(C)cccc2F)CC1]

48. The compound of any one of claims 1-39, or a pharmaceutically acceptable salt thereof, wherein R² is optionally substituted heterocyclyl.

49. The compound of any one of claims 1-39 or 48, or a pharmaceutically acceptable salt thereof, wherein R² is optionally substituted 4-7 membered heterocyclyl comprising 1 or 2 nitrogen atoms in the ring.

50. The compound of any one of claims 1-39, 48, or 49, or a pharmaceutically acceptable salt thereof, wherein R² is optionally substituted azetidine, pyrrolidine, piperidine, or piperazine.

51. The compound of any one of claims 1-39 or 48-50, or a pharmaceutically acceptable salt thereof, wherein R² is optionally substituted piperidine.

52. The compound of any one of claims 1-39 or 48-51, or a pharmaceutically acceptable salt thereof, wherein R² is 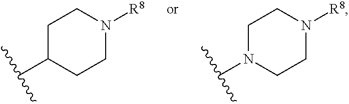
wherein R⁸ is optionally substituted alkyl, optionally substituted aryl, optionally substituted heteroaryl, or a nitrogen protecting group.

53. The compound of any one of claims 1-39 or 48-52, or a pharmaceutically acceptable salt thereof, wherein R² is

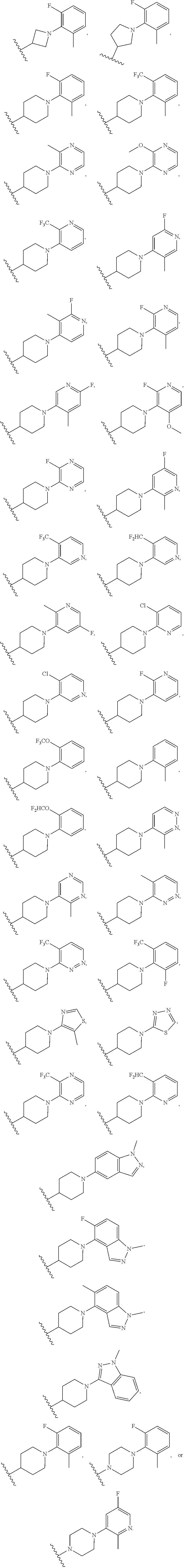

54. The compound of any one of claims 1-39 or 48-53, or a pharmaceutically acceptable salt thereof, wherein R² is

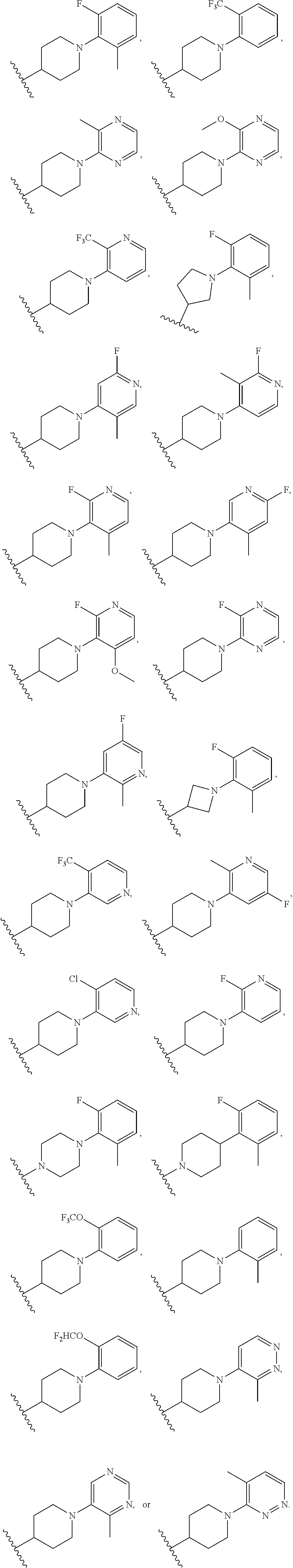

55. The compound of claim 1, wherein the compound is of Formula (I-a): 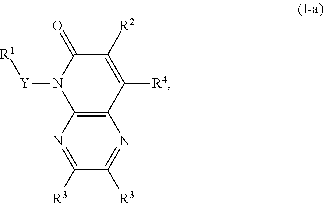
or a pharmaceutically acceptable salt thereof.

56. The compound of claim 1, wherein the compound is of Formula (I-a-1): 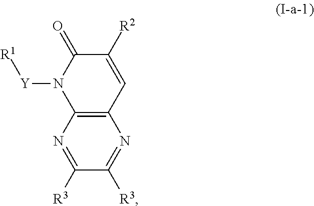
or a pharmaceutically acceptable salt thereof.

57. The compound of claim 1, wherein the compound is of Formula (I-a-2): 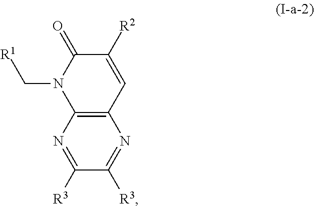
or a pharmaceutically acceptable salt thereof.

58. The compound of claim 1, wherein the compound is of Formula (I-a-3): 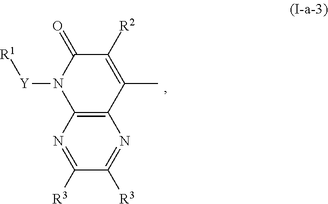
or a pharmaceutically acceptable salt thereof.

59. The compound of claim 1, wherein the compound is of Formula (I-a-4): 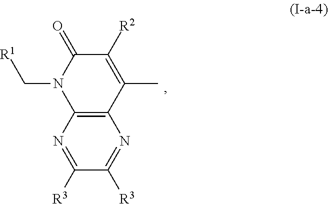
or a pharmaceutically acceptable salt thereof.

60. The compound of claim 1, wherein the compound is of Formula (I-a-5): 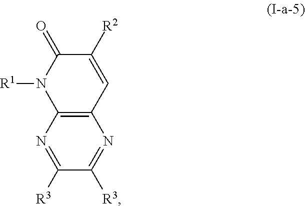
or a pharmaceutically acceptable salt thereof.

61. The compound of claim 1, wherein the compound is of Formula (I-a-6): 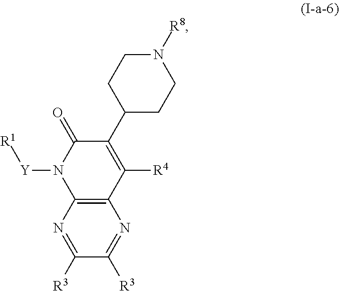
or a pharmaceutically acceptable salt thereof, wherein:
R⁸ is optionally substituted alkyl, optionally substituted aryl, optionally substituted heteroaryl, or a nitrogen protecting group.

62. The compound of claim 1, wherein the compound is of Formula (I-a-7): 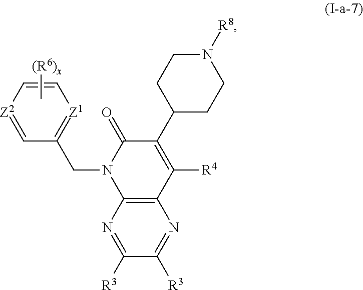
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A};
x is 0, 1, 2, or 3; and
R⁸ is optionally substituted alkyl, optionally substituted aryl, optionally substituted heteroaryl, or a nitrogen protecting group.

63. The compound of claim 1, wherein the compound is of Formula (I-a-8): 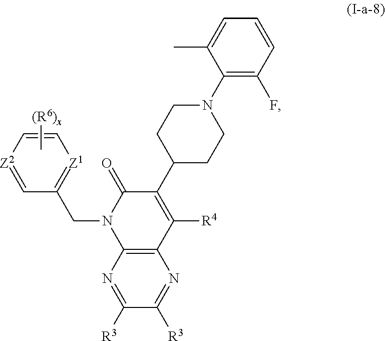
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A}; and
x is 0, 1, 2, or 3.

64. The compound of claim 1, wherein the compound is of Formula (I-a-9): 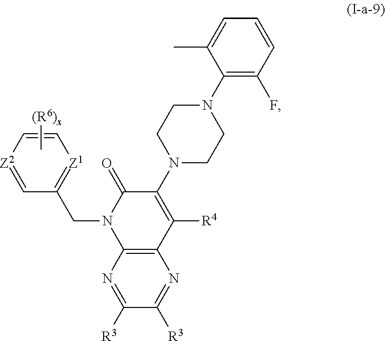
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A}; and
x is 0, 1, 2, or 3.

65. The compound of claim 1, wherein the compound is of Formula (I-a-10): 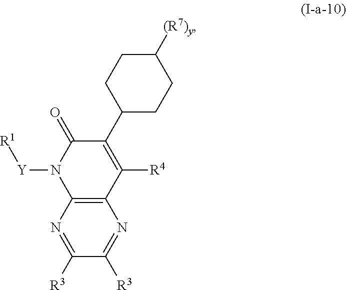
or a pharmaceutically acceptable salt thereof, wherein:
each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

66. The compound of claim 1, wherein the compound is of Formula (I-a-11): 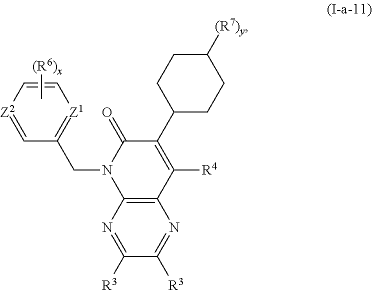
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A};
x is 0, 1, 2, or 3;
each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

67. The compound of claim 1, wherein the compound is of Formula (I-a-12): 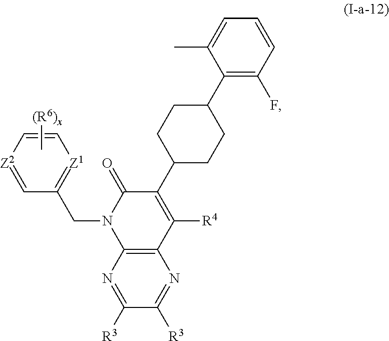
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A}; and
x is 0, 1, 2, or 3.

68. The compound of claim 1, wherein the compound is of Formula (I-b): 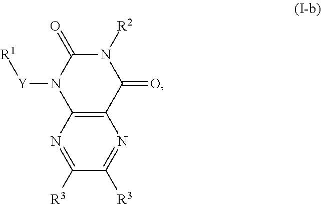
or a pharmaceutically acceptable salt thereof.

69. The compound of claim 1, wherein the compound is of Formula (I-c): 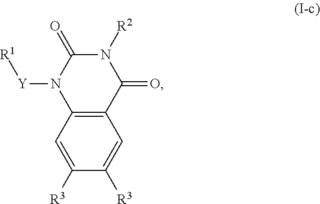
or a pharmaceutically acceptable salt thereof.

70. The compound of claim 1, wherein the compound is of Formula (I-d): 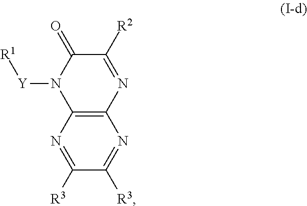
or a pharmaceutically acceptable salt thereof.

71. The compound of claim 1, wherein the compound is of Formula (I-e): 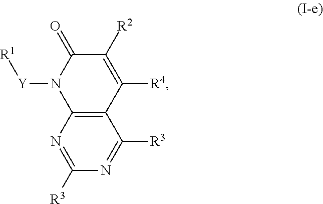
or a pharmaceutically acceptable salt thereof.

72. The compound of claim 1, wherein the compound is of Formula (I-e-1): 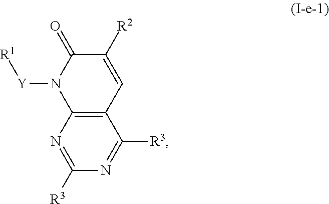
or a pharmaceutically acceptable salt thereof.

73. The compound of claim 1, wherein the compound is of Formula (I-e-2): 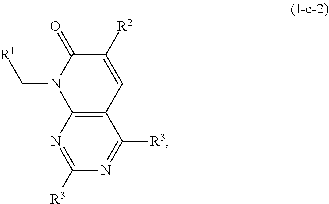
or a pharmaceutically acceptable salt thereof.

74. The compound of claim 1, wherein the compound is of Formula (I-e-3): 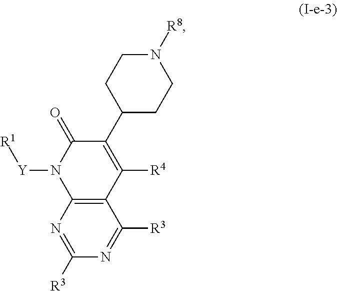
or a pharmaceutically acceptable salt thereof, wherein:
R⁸ is optionally substituted alkyl, optionally substituted aryl, optionally substituted heteroaryl, or a nitrogen protecting group.

75. The compound of claim 1, wherein the compound is of Formula (I-e-4): 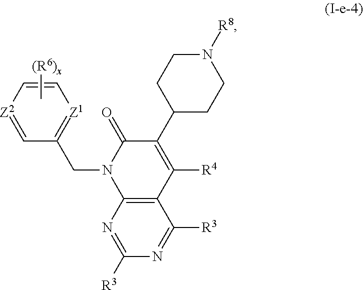
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A};
x is 0, 1, 2, or 3; and
R⁸ is optionally substituted alkyl, optionally substituted aryl, optionally substituted heteroaryl, or a nitrogen protecting group.

76. The compound of claim 1, wherein the compound is of Formula (I-e-5): 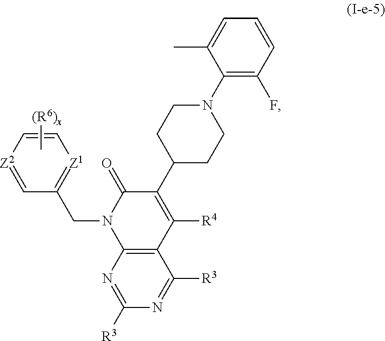
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A}; and
x is 0, 1, 2, or 3.

77. The compound of claim 1, wherein the compound is of Formula (I-e-6): 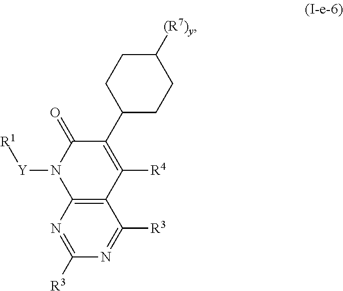
or a pharmaceutically acceptable salt thereof, wherein:
each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

78. The compound of claim 1, wherein the compound is of Formula (I-e-7): 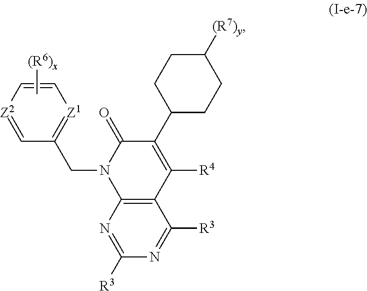
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A};
x is 0, 1, 2, or 3;
each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

79. The compound of claim 1, wherein the compound is of Formula (I-e-8): 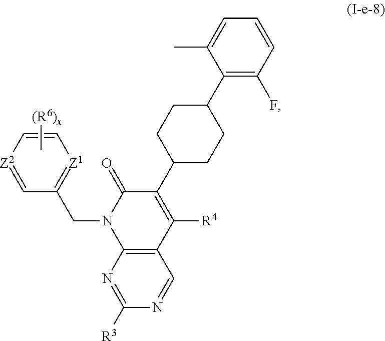
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A}; and
x is 0, 1, 2, or 3.

80. The compound of claim 1, wherein the compound is of Formula (I-f): 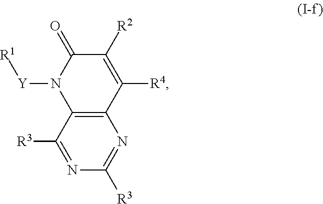
or a pharmaceutically acceptable salt thereof.

81. The compound of claim 1, wherein the compound is of Formula (I-f-1): 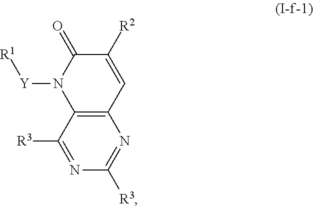
or a pharmaceutically acceptable salt thereof.

82. The compound of claim 1, wherein the compound is of Formula (I-f-2): 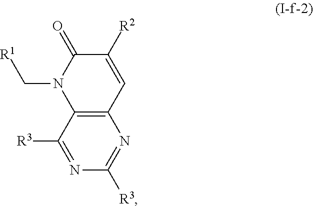
or a pharmaceutically acceptable salt thereof.

83. The compound of claim 1, wherein the compound is of Formula (I-g): 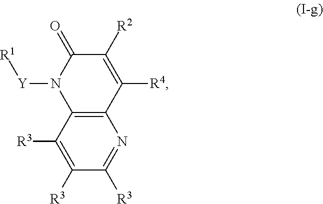
or a pharmaceutically acceptable salt thereof.

84. The compound of claim 1, wherein the compound is of Formula (I-g-1): 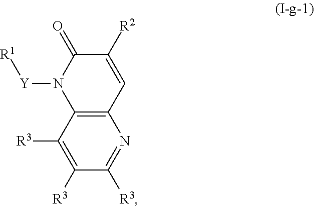
or a pharmaceutically acceptable salt thereof.

85. The compound of claim 1, wherein the compound is of Formula (I-g-2): 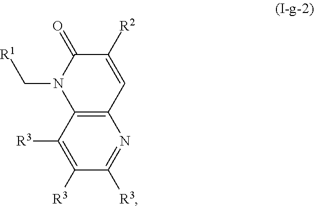
or a pharmaceutically acceptable salt thereof.

86. The compound of claim 1, wherein the compound is of Formula (I-h): 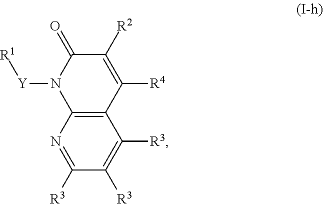
or a pharmaceutically acceptable salt thereof.

87. The compound of claim 1, wherein the compound is of Formula (I-h-1): 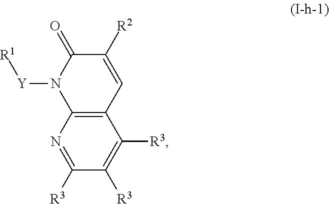
or a pharmaceutically acceptable salt thereof.

88. The compound of claim 1, wherein the compound is of Formula (I-h-2): 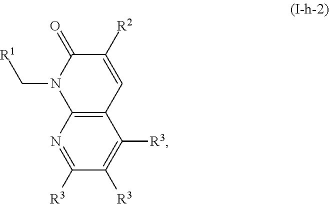
or a pharmaceutically acceptable salt thereof.

89. The compound of claim 1, wherein the compound is of Formula (I-h-7): 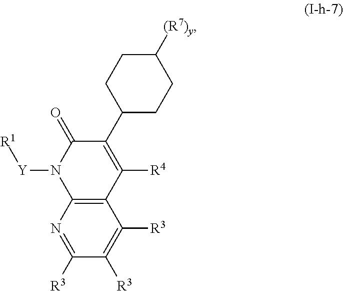
or a pharmaceutically acceptable salt thereof, wherein:
each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

90. The compound of claim 1, wherein the compound is of Formula (I-h-8): 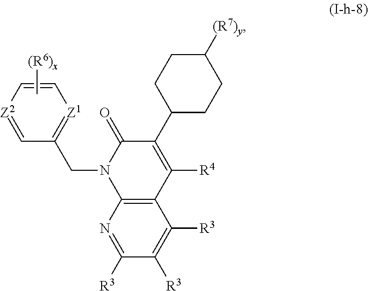
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A};
x is 0, 1, 2, or 3;
each R⁷ is independently halogen, optionally substituted alkyl, optionally substituted aryl, or optionally substituted heteroaryl, or two instances of R⁷ together with the atoms to which they are attached form a substituted or unsubstituted carbocyclyl or substituted or unsubstituted heterocyclyl; and y is 0, 1, or 2.

91. The compound of claim 1, wherein the compound is of Formula (I-h-9): 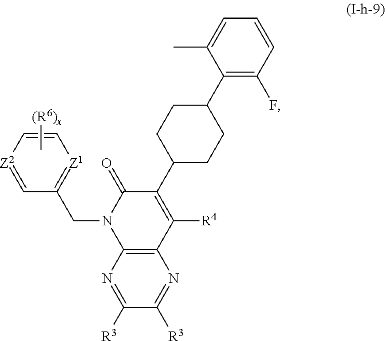
or a pharmaceutically acceptable salt thereof, wherein:
Z¹ and Z² are each independently CH, CR⁶, or N;
each R⁶ is independently halogen, optionally substituted alkyl, optionally substituted cycloalkyl, or —OR^{A}; and
x is 0, 1, 2, or 3.

92. The compound of claim 1, wherein the compound is 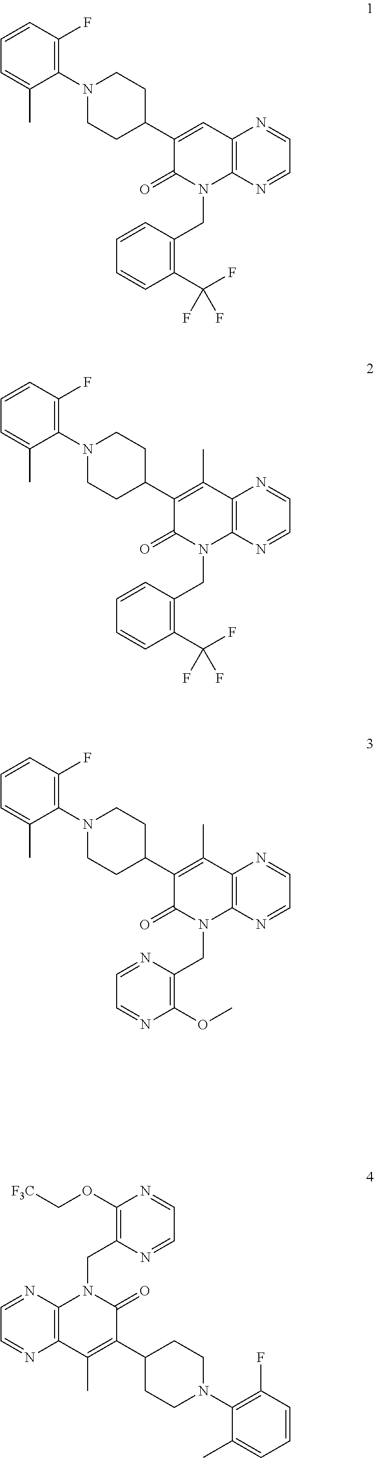 [SMILES: Cc1cccc(F)c1N1CCC(c2cc3nccnc3n(Cc3ccccc3C(F)(F)F)c2=O)CC1 ; Cc1cccc(F)c1N1CCC(c2c(C)c3nccnc3n(Cc3ccccc3C(F)(F)F)c2=O)CC1 ; COc1nccnc1Cn1c(=O)c(C2CCN(c3c(C)cccc3F)CC2)c(C)c2nccnc21 ; Cc1cccc(F)c1N1CCC(c2c(C)c3nccnc3n(Cc3nccnc3OCC(F)(F)F)c2=O)CC1] 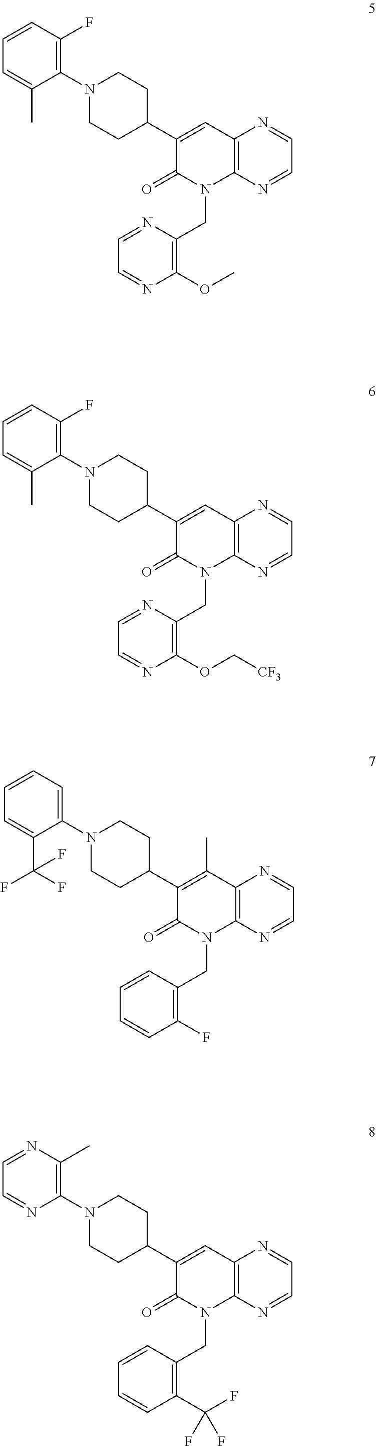 [SMILES: COc1nccnc1Cn1c(=O)c(C2CCN(c3c(C)cccc3F)CC2)cc2nccnc21 ; Cc1cccc(F)c1N1CCC(c2cc3nccnc3n(Cc3nccnc3OCC(F)(F)F)c2=O)CC1 ; Cc1c(C2CCN(c3ccccc3C(F)(F)F)CC2)c(=O)n(Cc2ccccc2F)c2nccnc12 ; Cc1nccnc1N1CCC(c2cc3nccnc3n(Cc3ccccc3C(F)(F)F)c2=O)CC1] 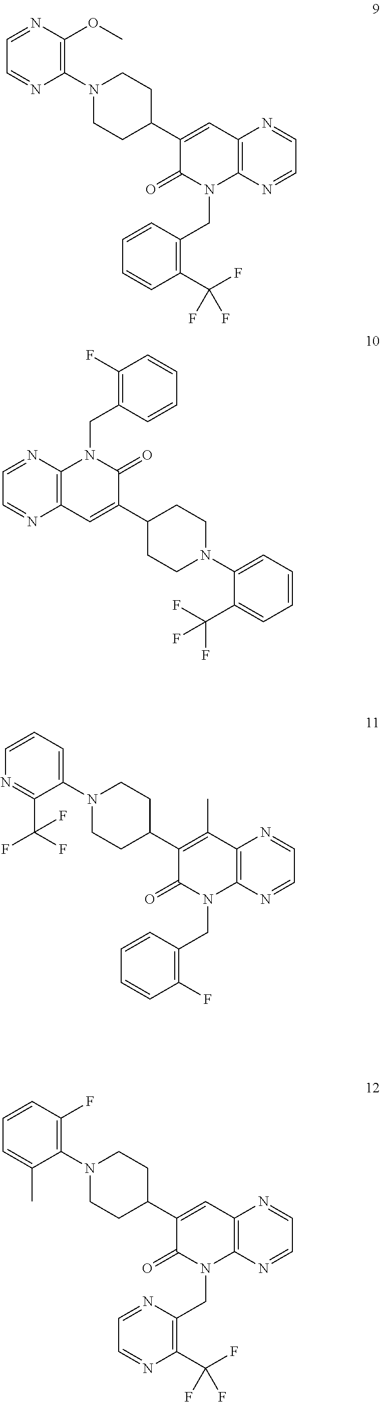 [SMILES: COc1nccnc1N1CCC(c2cc3nccnc3n(Cc3ccccc3C(F)(F)F)c2=O)CC1 ; O=c1c(C2CCN(c3ccccc3C(F)(F)F)CC2)cc2nccnc2n1Cc1ccccc1F ; Cc1c(C2CCN(c3cccnc3C(F)(F)F)CC2)c(=O)n(Cc2ccccc2F)c2nccnc12 ; Cc1cccc(F)c1N1CCC(c2cc3nccnc3n(Cc3nccnc3C(F)(F)F)c2=O)CC1] 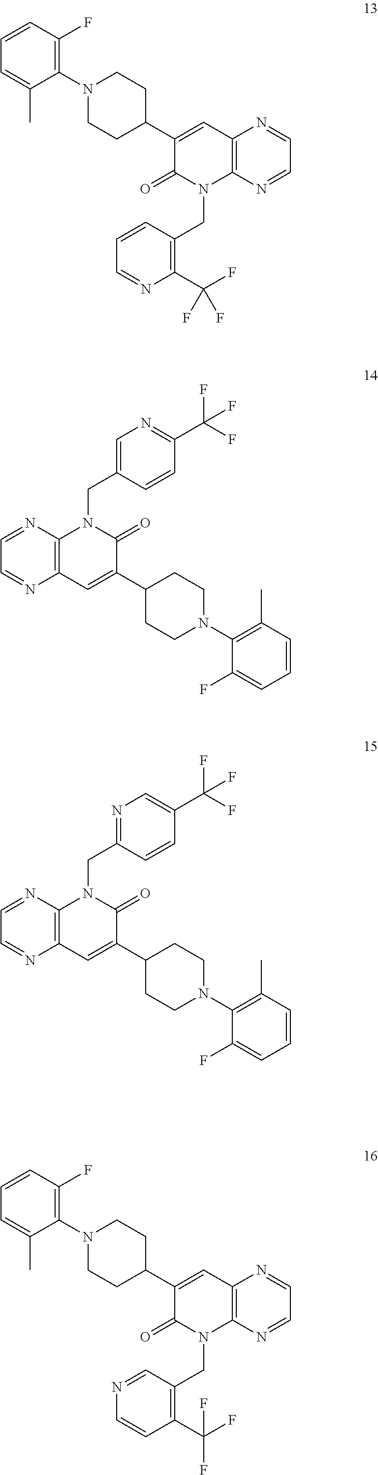 [SMILES: Cc1cccc(F)c1N1CCC(c2cc3nccnc3n(Cc3cccnc3C(F)(F)F)c2=O)CC1 ; Cc1cccc(F)c1N1CCC(c2cc3nccnc3n(Cc3ccc(C(F)(F)F)nc3)c2=O)CC1 ; Cc1cccc(F)c1N1CCC(c2cc3nccnc3n(Cc3ccc(C(F)(F)F)cn3)c2=O)CC1 ; Cc1cccc(F)c1N1CCC(c2cc3nccnc3n(Cc3cnccc3C(F)(F)F)c2=O)CC1] 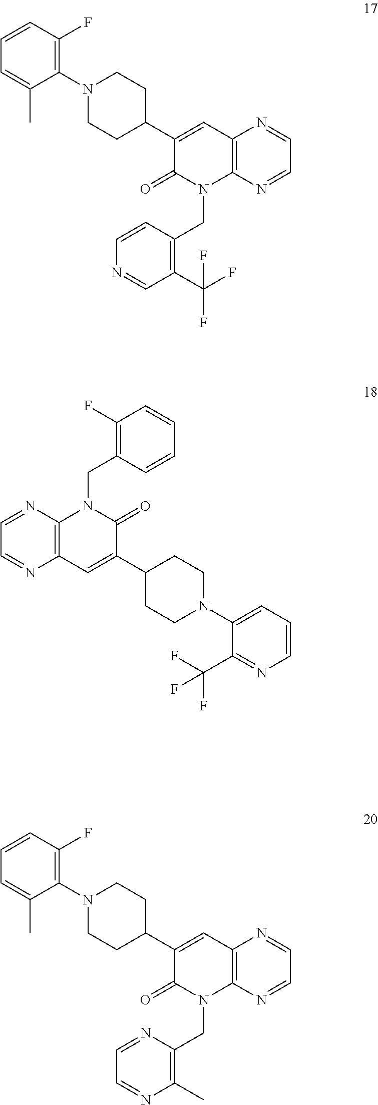 [SMILES: Cc1cccc(F)c1N1CCC(c2cc3nccnc3n(Cc3ccncc3C(F)(F)F)c2=O)CC1 ; O=c1c(C2CCN(c3cccnc3C(F)(F)F)CC2)cc2nccnc2n1Cc1ccccc1F ; Cc1cccc(F)c1N1CCC(c2cc3nccnc3n(Cc3nccnc3C)c2=O)CC1] 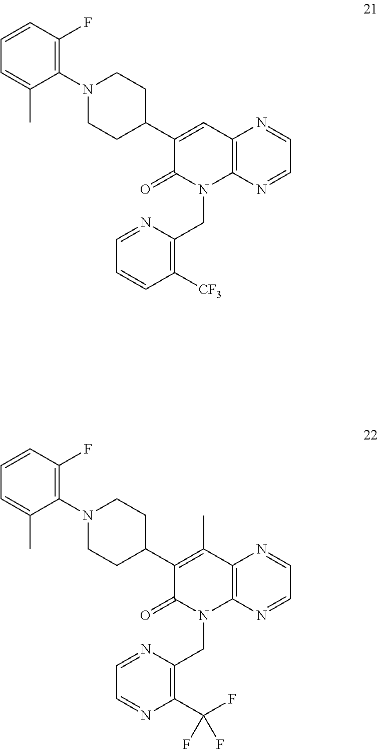 [SMILES: Cc1cccc(F)c1N1CCC(c2cc3nccnc3n(Cc3ncccc3C(F)(F)F)c2=O)CC1 ; Cc1cccc(F)c1N1CCC(c2c(C)c3nccnc3n(Cc3nccnc3C(F)(F)F)c2=O)CC1] 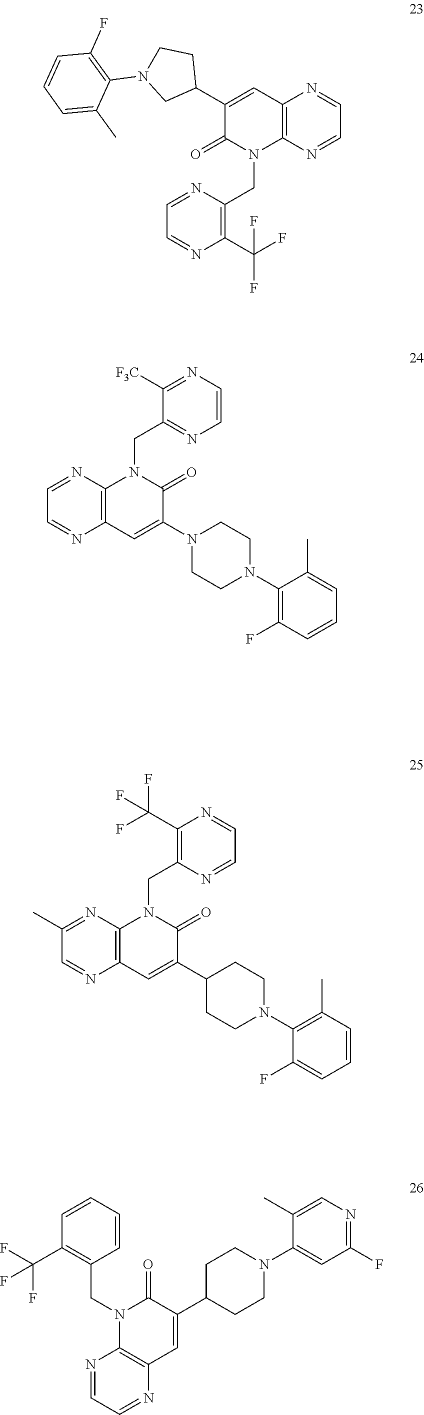 [SMILES: Cc1cccc(F)c1N1CCC(c2cc3nccnc3n(Cc3nccnc3C(F)(F)F)c2=O)C1 ; Cc1cccc(F)c1N1CCN(c2cc3nccnc3n(Cc3nccnc3C(F)(F)F)c2=O)CC1 ; Cc1cnc2cc(C3CCN(c4c(C)cccc4F)CC3)c(=O)n(Cc3nccnc3C(F)(F)F)c2n1 ; Cc1cnc(F)cc1N1CCC(c2cc3nccnc3n(Cc3ccccc3C(F)(F)F)c2=O)CC1] 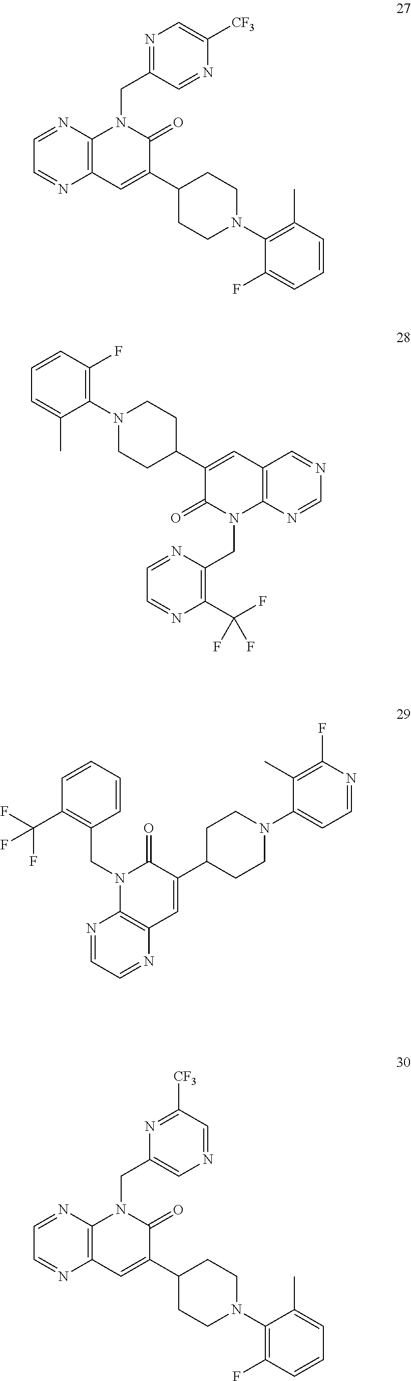 [SMILES: Cc1cccc(F)c1N1CCC(c2cc3nccnc3n(Cc3cnc(C(F)(F)F)cn3)c2=O)CC1 ; Cc1cccc(F)c1N1CCC(c2cc3cncnc3n(Cc3nccnc3C(F)(F)F)c2=O)CC1 ; Cc1c(N2CCC(c3cc4nccnc4n(Cc4ccccc4C(F)(F)F)c3=O)CC2)ccnc1F ; Cc1cccc(F)c1N1CCC(c2cc3nccnc3n(Cc3cncc(C(F)(F)F)n3)c2=O)CC1] 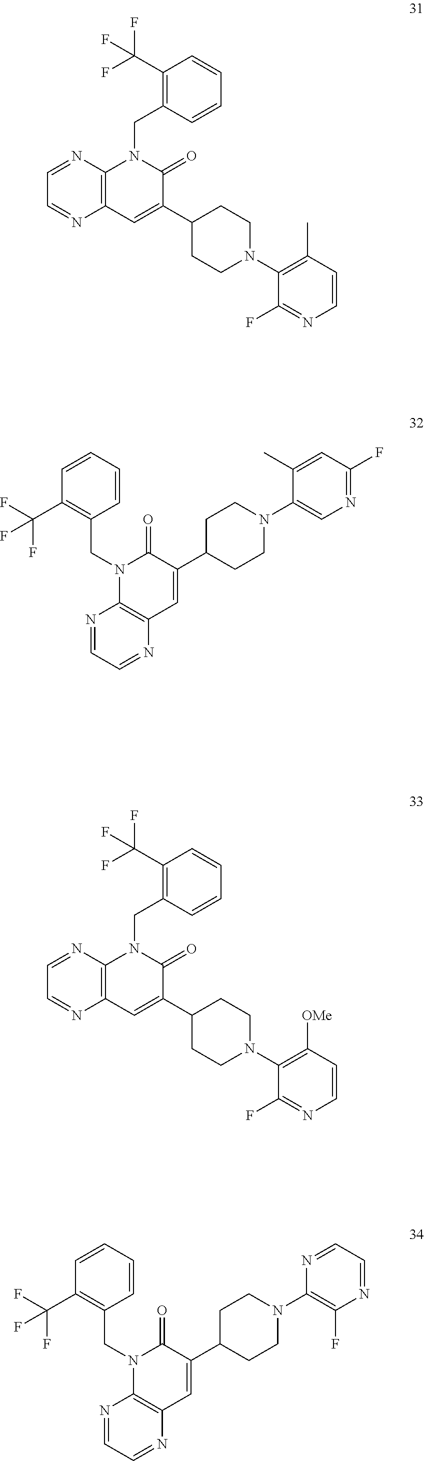 [SMILES: Cc1ccnc(F)c1N1CCC(c2cc3nccnc3n(Cc3ccccc3C(F)(F)F)c2=O)CC1 ; Cc1cc(F)ncc1N1CCC(c2cc3nccnc3n(Cc3ccccc3C(F)(F)F)c2=O)CC1 ; COc1ccnc(F)c1N1CCC(c2cc3nccnc3n(Cc3ccccc3C(F)(F)F)c2=O)CC1 ; O=c1c(C2CCN(c3nccnc3F)CC2)cc2nccnc2n1Cc1ccccc1C(F)(F)F] 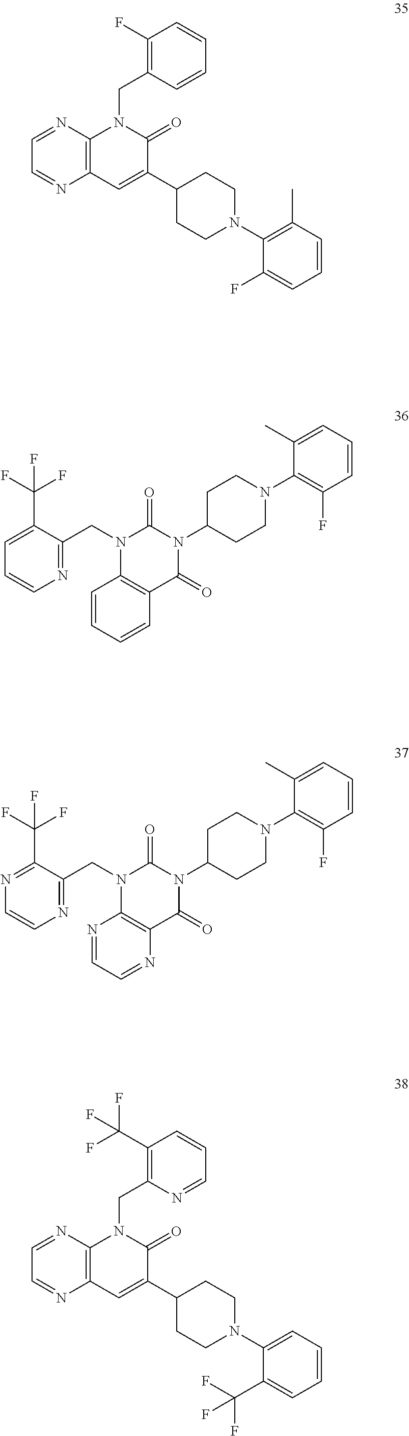 [SMILES: Cc1cccc(F)c1N1CCC(c2cc3nccnc3n(Cc3ccccc3F)c2=O)CC1 ; Cc1cccc(F)c1N1CCC(n2c(=O)c3ccccc3n(Cc3ncccc3C(F)(F)F)c2=O)CC1 ; Cc1cccc(F)c1N1CCC(n2c(=O)c3nccnc3n(Cc3nccnc3C(F)(F)F)c2=O)CC1 ; O=c1c(C2CCN(c3ccccc3C(F)(F)F)CC2)cc2nccnc2n1Cc1ncccc1C(F)(F)F] 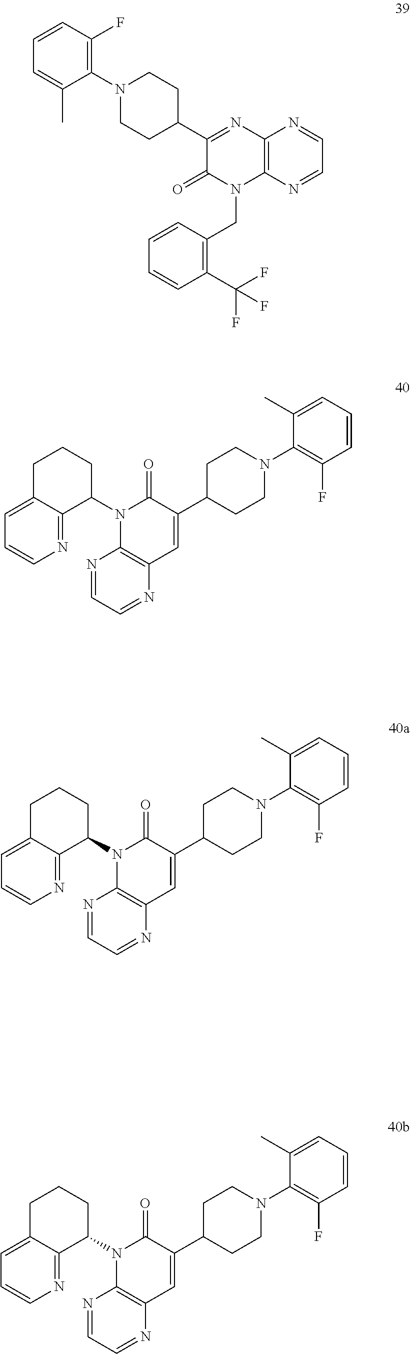 [SMILES: Cc1cccc(F)c1N1CCC(c2nc3nccnc3n(Cc3ccccc3C(F)(F)F)c2=O)CC1 ; Cc1cccc(F)c1N1CCC(c2cc3nccnc3n(C3CCCc4cccnc43)c2=O)CC1 ; Cc1cccc(F)c1N1CCC(c2cc3nccnc3n([C@@H]3CCCc4cccnc43)c2=O)CC1 ; Cc1cccc(F)c1N1CCC(c2cc3nccnc3n([C@H]3CCCc4cccnc43)c2=O)CC1] 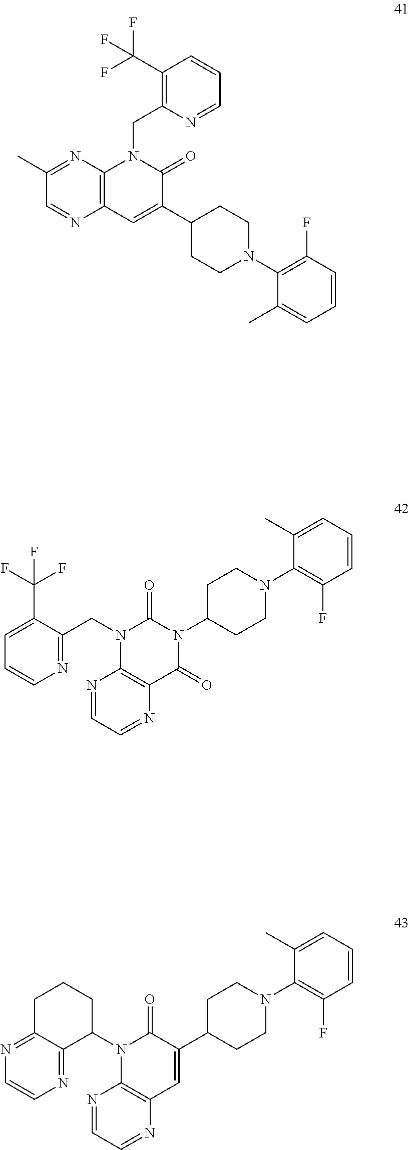 [SMILES: Cc1cnc2cc(C3CCN(c4c(C)cccc4F)CC3)c(=O)n(Cc3ncccc3C(F)(F)F)c2n1 ; Cc1cccc(F)c1N1CCC(n2c(=O)c3nccnc3n(Cc3ncccc3C(F)(F)F)c2=O)CC1 ; Cc1cccc(F)c1N1CCC(c2cc3nccnc3n(C3CCCc4nccnc43)c2=O)CC1] 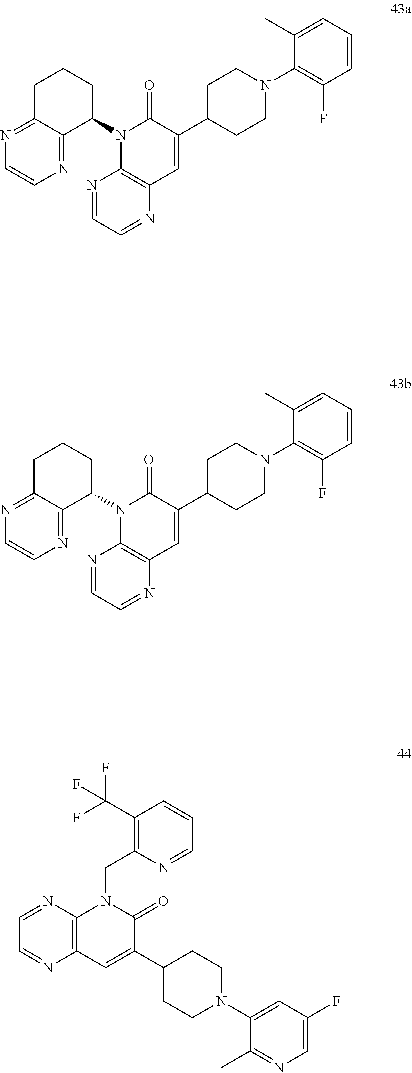 [SMILES: Cc1cccc(F)c1N1CCC(c2cc3nccnc3n([C@@H]3CCCc4nccnc43)c2=O)CC1 ; Cc1cccc(F)c1N1CCC(c2cc3nccnc3n([C@H]3CCCc4nccnc43)c2=O)CC1 ; Cc1ncc(F)cc1N1CCC(c2cc3nccnc3n(Cc3ncccc3C(F)(F)F)c2=O)CC1] 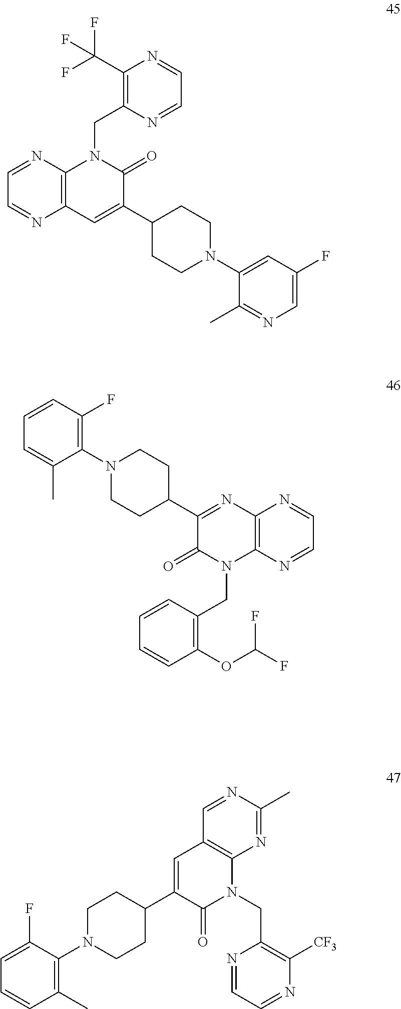 [SMILES: Cc1ncc(F)cc1N1CCC(c2cc3nccnc3n(Cc3nccnc3C(F)(F)F)c2=O)CC1 ; Cc1cccc(F)c1N1CCC(c2nc3nccnc3n(Cc3ccccc3OC(F)F)c2=O)CC1 ; Cc1ncc2cc(C3CCN(c4c(C)cccc4F)CC3)c(=O)n(Cc3nccnc3C(F)(F)F)c2n1] 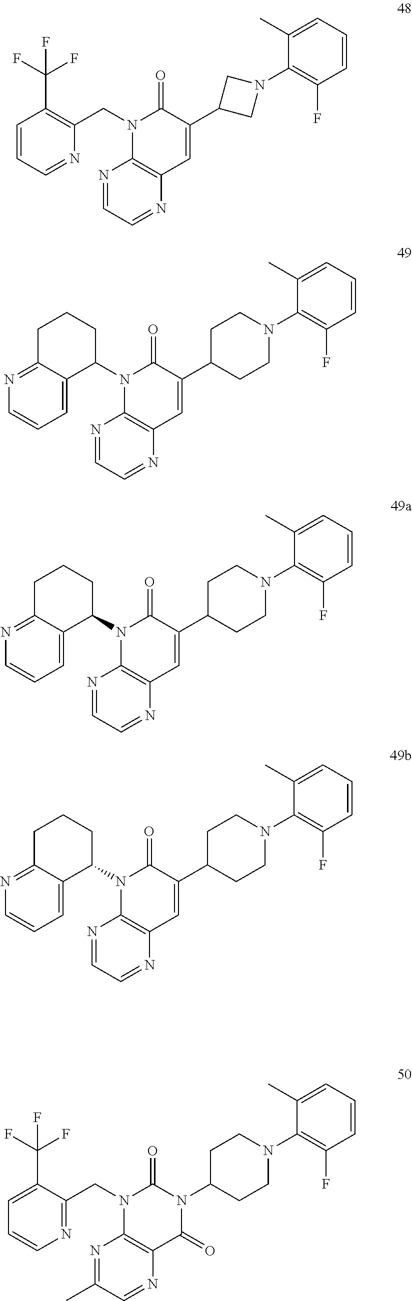 [SMILES: Cc1cccc(F)c1N1CC(c2cc3nccnc3n(Cc3ncccc3C(F)(F)F)c2=O)C1 ; Cc1cccc(F)c1N1CCC(c2cc3nccnc3n(C3CCCc4ncccc43)c2=O)CC1 ; Cc1cccc(F)c1N1CCC(c2cc3nccnc3n([C@@H]3CCCc4ncccc43)c2=O)CC1 ; Cc1cccc(F)c1N1CCC(c2cc3nccnc3n([C@H]3CCCc4ncccc43)c2=O)CC1 ; Cc1cnc2c(=O)n(C3CCN(c4c(C)cccc4F)CC3)c(=O)n(Cc3ncccc3C(F)(F)F)c2n1] 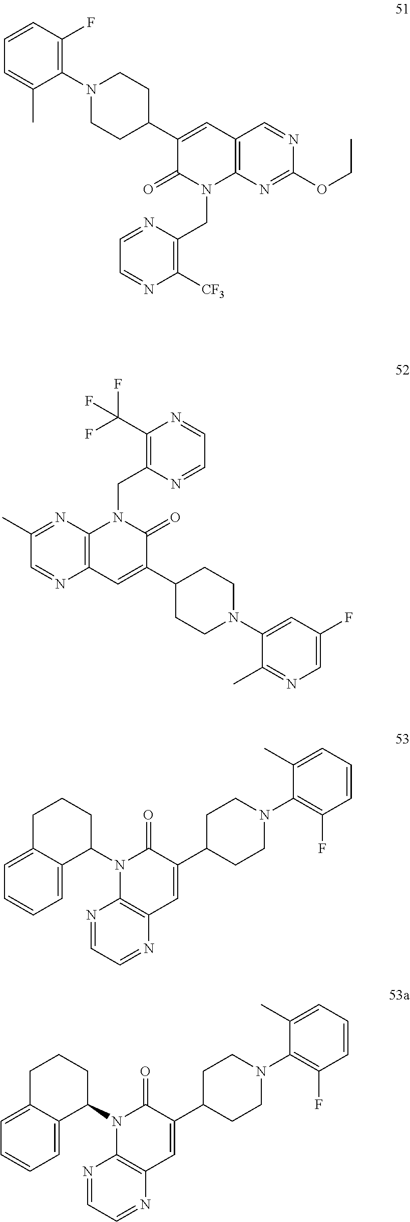 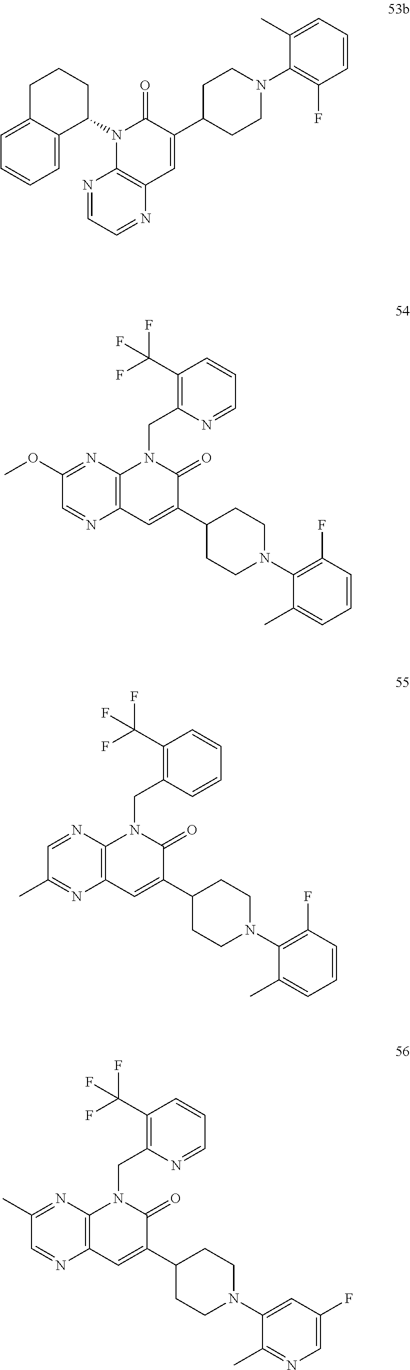 [SMILES: Cc1cccc(F)c1N1CCC(c2cc3nccnc3n([C@H]3CCCc4ccccc43)c2=O)CC1 ; COc1cnc2cc(C3CCN(c4c(C)cccc4F)CC3)c(=O)n(Cc3ncccc3C(F)(F)F)c2n1 ; Cc1cnc2c(cc(C3CCN(c4c(C)cccc4F)CC3)c(=O)n2Cc2ccccc2C(F)(F)F)n1 ; Cc1cnc2cc(C3CCN(c4cc(F)cnc4C)CC3)c(=O)n(Cc3ncccc3C(F)(F)F)c2n1] 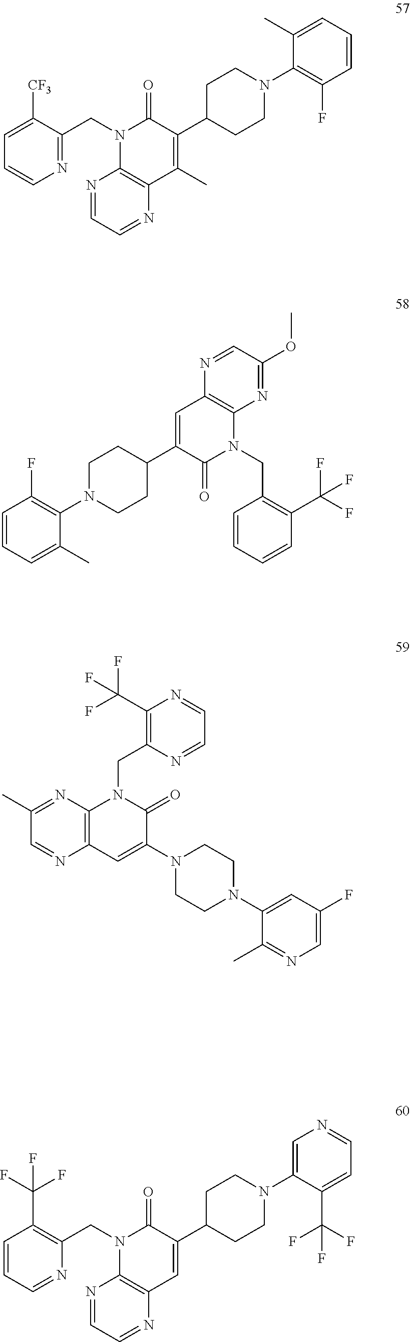 [SMILES: Cc1cccc(F)c1N1CCC(c2c(C)c3nccnc3n(Cc3ncccc3C(F)(F)F)c2=O)CC1 ; COc1cnc2cc(C3CCN(c4c(C)cccc4F)CC3)c(=O)n(Cc3ccccc3C(F)(F)F)c2n1 ; Cc1cnc2cc(N3CCN(c4cc(F)cnc4C)CC3)c(=O)n(Cc3nccnc3C(F)(F)F)c2n1 ; O=c1c(C2CCN(c3cnccc3C(F)(F)F)CC2)cc2nccnc2n1Cc1ncccc1C(F)(F)F] 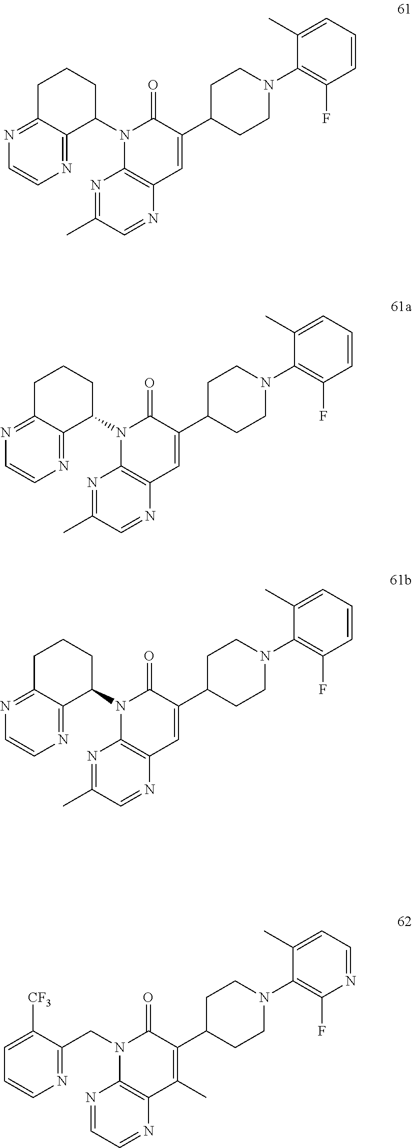 [SMILES: Cc1cnc2cc(C3CCN(c4c(C)cccc4F)CC3)c(=O)n(C3CCCc4nccnc43)c2n1 ; Cc1cnc2cc(C3CCN(c4c(C)cccc4F)CC3)c(=O)n([C@H]3CCCc4nccnc43)c2n1 ; Cc1cnc2cc(C3CCN(c4c(C)cccc4F)CC3)c(=O)n([C@@H]3CCCc4nccnc43)c2n1 ; Cc1ccnc(F)c1N1CCC(c2c(C)c3nccnc3n(Cc3ncccc3C(F)(F)F)c2=O)CC1] 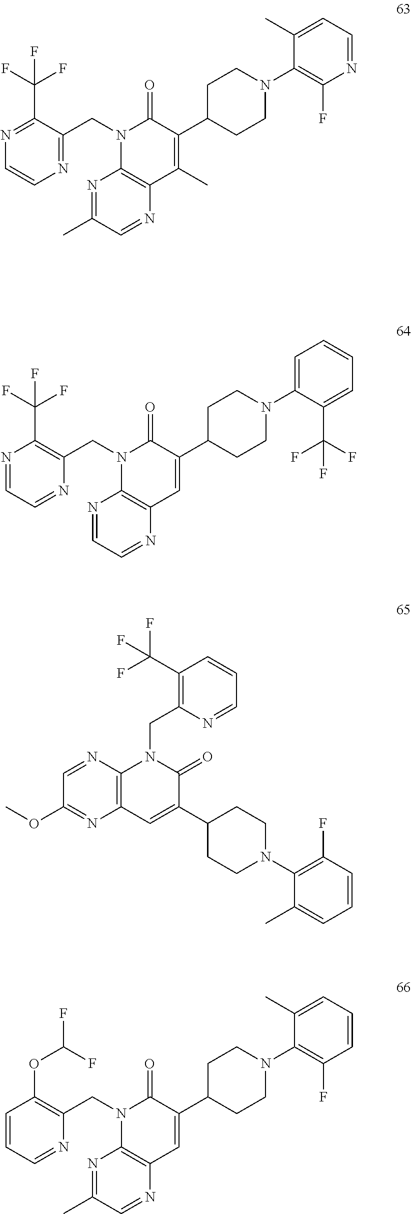 [SMILES: Cc1cnc2c(C)c(C3CCN(c4c(C)ccnc4F)CC3)c(=O)n(Cc3nccnc3C(F)(F)F)c2n1 ; O=c1c(C2CCN(c3ccccc3C(F)(F)F)CC2)cc2nccnc2n1Cc1nccnc1C(F)(F)F ; COc1cnc2c(cc(C3CCN(c4c(C)cccc4F)CC3)c(=O)n2Cc2ncccc2C(F)(F)F)n1 ; Cc1cnc2cc(C3CCN(c4c(C)cccc4F)CC3)c(=O)n(Cc3ncccc3OC(F)F)c2n1] 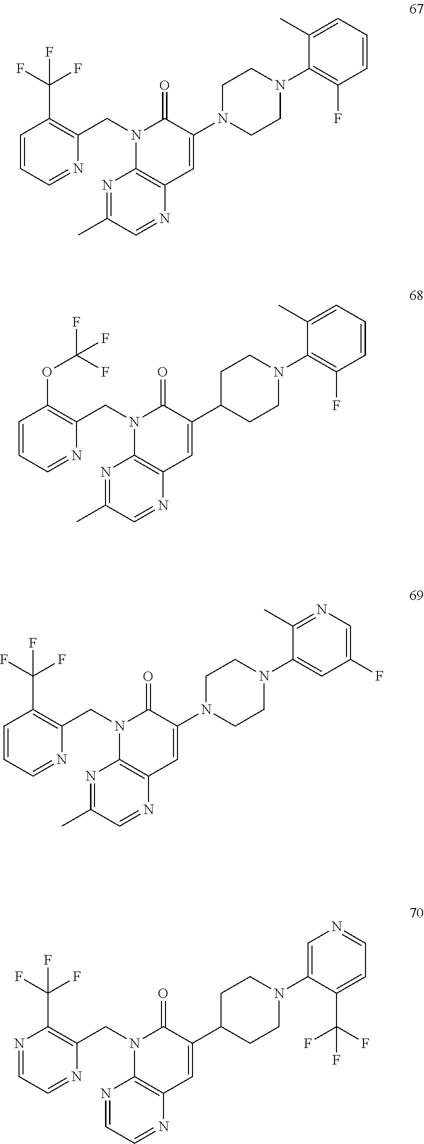 [SMILES: Cc1cnc2cc(N3CCN(c4c(C)cccc4F)CC3)c(=O)n(Cc3ncccc3C(F)(F)F)c2n1 ; Cc1cnc2cc(C3CCN(c4c(C)cccc4F)CC3)c(=O)n(Cc3ncccc3OC(F)(F)F)c2n1 ; Cc1cnc2cc(N3CCN(c4cc(F)cnc4C)CC3)c(=O)n(Cc3ncccc3C(F)(F)F)c2n1 ; O=c1c(C2CCN(c3cnccc3C(F)(F)F)CC2)cc2nccnc2n1Cc1nccnc1C(F)(F)F] 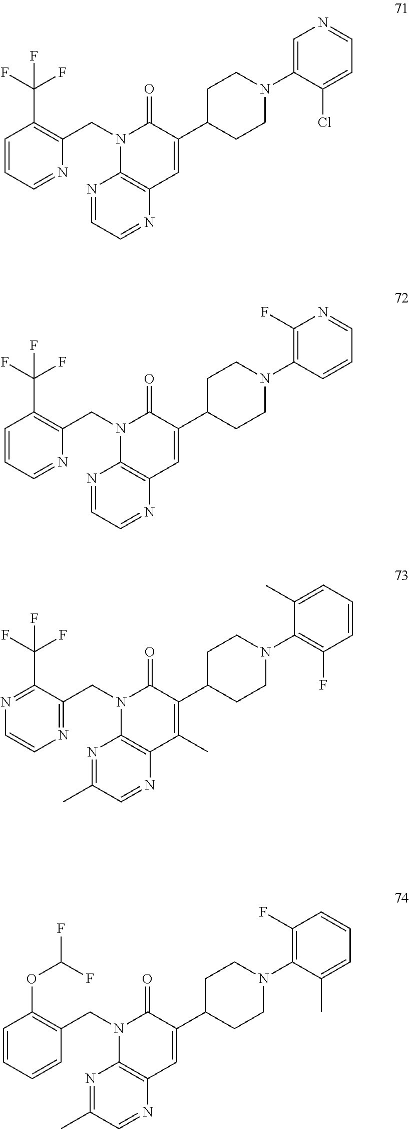 [SMILES: O=c1c(C2CCN(c3cnccc3Cl)CC2)cc2nccnc2n1Cc1ncccc1C(F)(F)F ; O=c1c(C2CCN(c3cccnc3F)CC2)cc2nccnc2n1Cc1ncccc1C(F)(F)F ; Cc1cnc2c(C)c(C3CCN(c4c(C)cccc4F)CC3)c(=O)n(Cc3nccnc3C(F)(F)F)c2n1 ; Cc1cnc2cc(C3CCN(c4c(C)cccc4F)CC3)c(=O)n(Cc3ccccc3OC(F)F)c2n1] 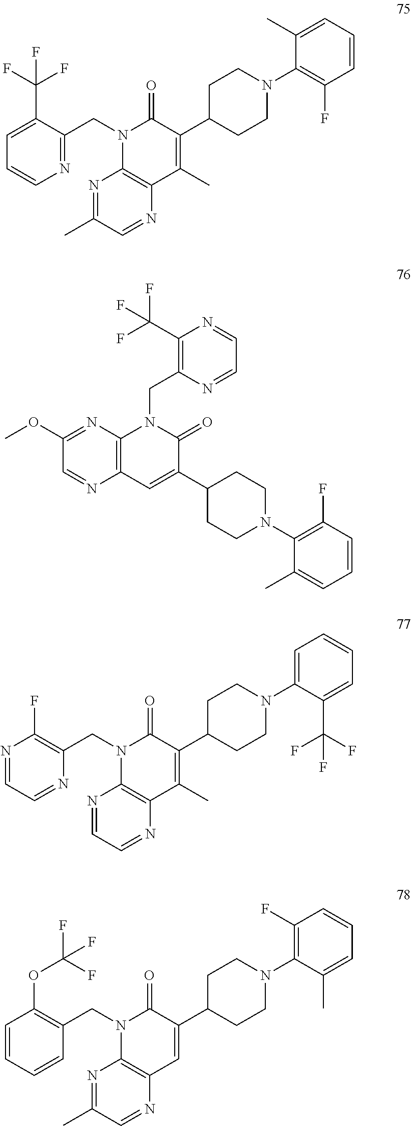 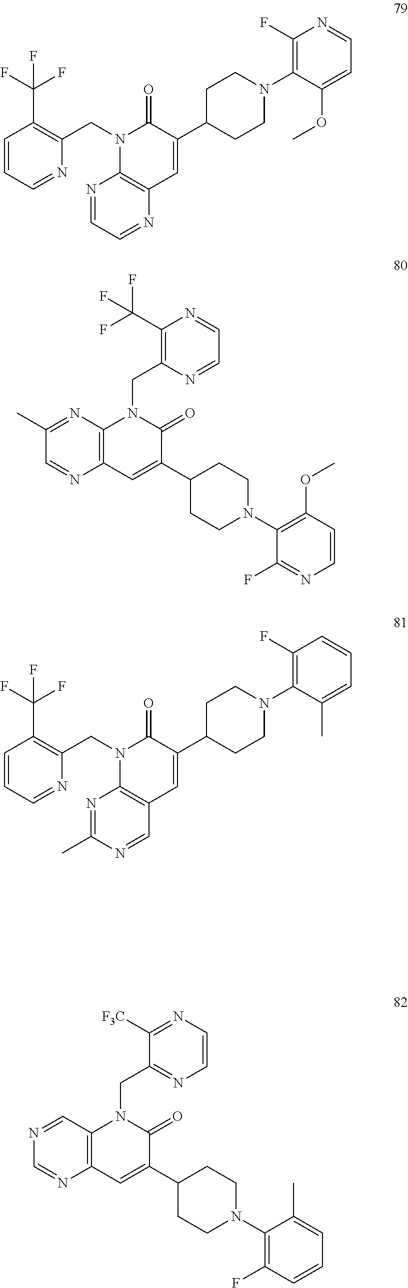 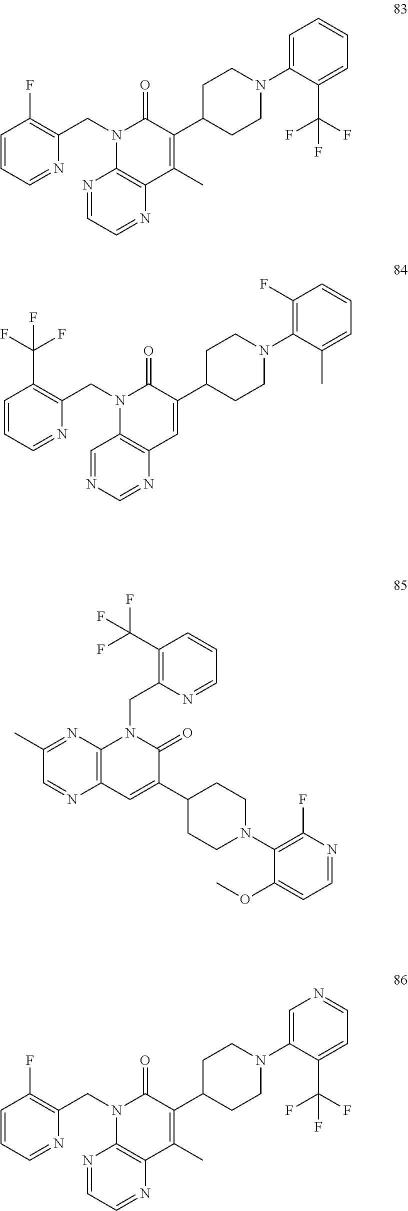 [SMILES: Cc1c(C2CCN(c3ccccc3C(F)(F)F)CC2)c(=O)n(Cc2ncccc2F)c2nccnc12 ; Cc1cccc(F)c1N1CCC(c2cc3ncncc3n(Cc3ncccc3C(F)(F)F)c2=O)CC1 ; COc1ccnc(F)c1N1CCC(c2cc3ncc(C)nc3n(Cc3ncccc3C(F)(F)F)c2=O)CC1 ; Cc1c(C2CCN(c3cnccc3C(F)(F)F)CC2)c(=O)n(Cc2ncccc2F)c2nccnc12] 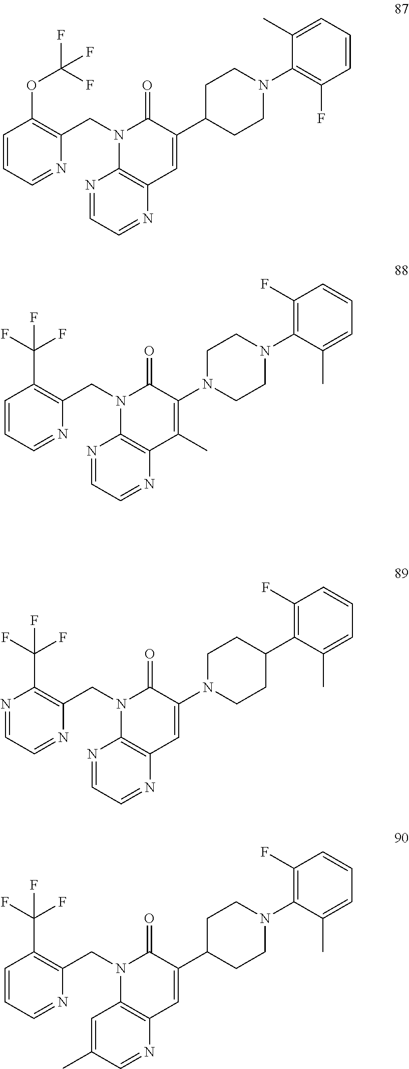 [SMILES: Cc1cccc(F)c1N1CCC(c2cc3nccnc3n(Cc3ncccc3OC(F)(F)F)c2=O)CC1 ; Cc1cccc(F)c1N1CCN(c2c(C)c3nccnc3n(Cc3ncccc3C(F)(F)F)c2=O)CC1 ; Cc1cccc(F)c1C1CCN(c2cc3nccnc3n(Cc3nccnc3C(F)(F)F)c2=O)CC1 ; Cc1cnc2cc(C3CCN(c4c(C)cccc4F)CC3)c(=O)n(Cc3ncccc3C(F)(F)F)c2c1] 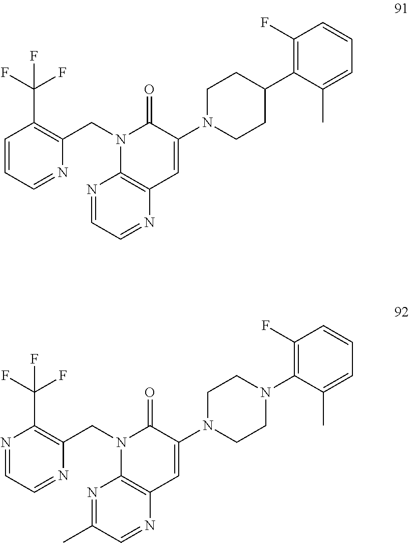 [SMILES: Cc1cccc(F)c1C1CCN(c2cc3nccnc3n(Cc3ncccc3C(F)(F)F)c2=O)CC1 ; Cc1cnc2cc(N3CCN(c4c(C)cccc4F)CC3)c(=O)n(Cc3nccnc3C(F)(F)F)c2n1] 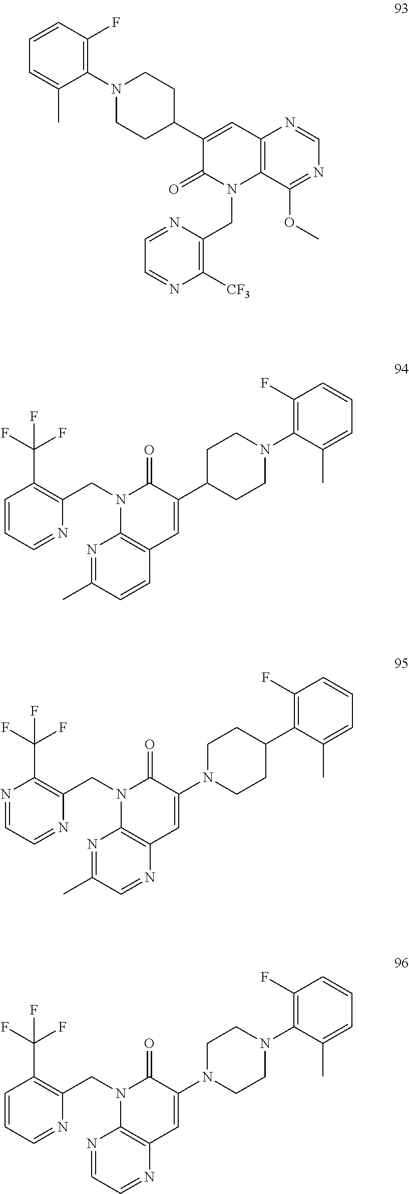 [SMILES: COc1ncnc2cc(C3CCN(c4c(C)cccc4F)CC3)c(=O)n(Cc3nccnc3C(F)(F)F)c12 ; Cc1ccc2cc(C3CCN(c4c(C)cccc4F)CC3)c(=O)n(Cc3ncccc3C(F)(F)F)c2n1 ; Cc1cnc2cc(N3CCC(c4c(C)cccc4F)CC3)c(=O)n(Cc3nccnc3C(F)(F)F)c2n1 ; Cc1cccc(F)c1N1CCN(c2cc3nccnc3n(Cc3ncccc3C(F)(F)F)c2=O)CC1] 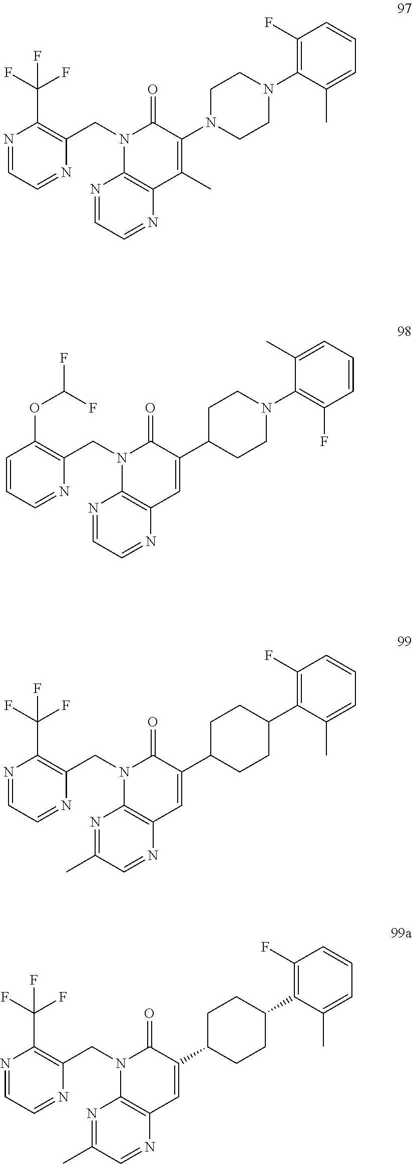 [SMILES: Cc1cccc(F)c1N1CCN(c2c(C)c3nccnc3n(Cc3nccnc3C(F)(F)F)c2=O)CC1 ; Cc1cccc(F)c1N1CCC(c2cc3nccnc3n(Cc3ncccc3OC(F)F)c2=O)CC1 ; Cc1cnc2cc(C3CCC(c4c(C)cccc4F)CC3)c(=O)n(Cc3nccnc3C(F)(F)F)c2n1 ; Cc1cnc2cc([C@H]3CC[C@@H](c4c(C)cccc4F)CC3)c(=O)n(Cc3nccnc3C(F)(F)F)c2n1] 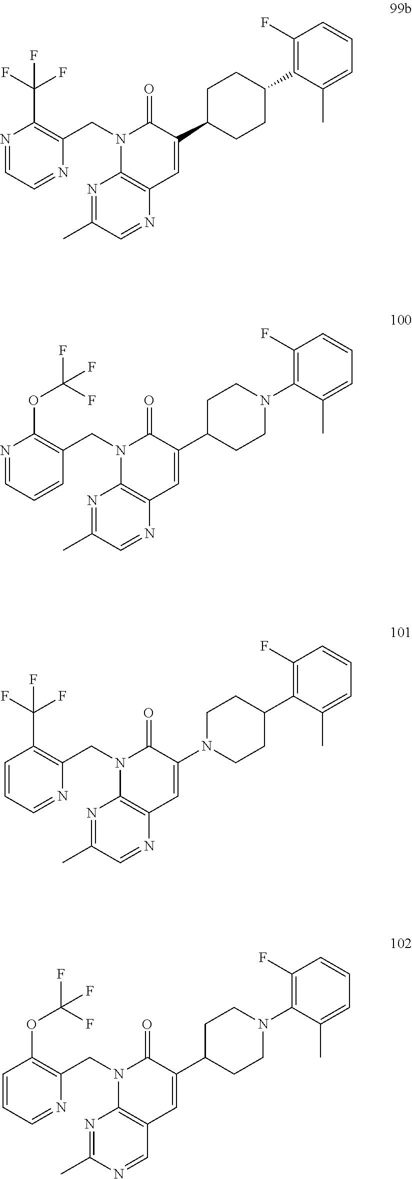 [SMILES: Cc1cnc2cc([C@H]3CC[C@H](c4c(C)cccc4F)CC3)c(=O)n(Cc3nccnc3C(F)(F)F)c2n1 ; Cc1cnc2cc(C3CCN(c4c(C)cccc4F)CC3)c(=O)n(Cc3cccnc3OC(F)(F)F)c2n1 ; Cc1cnc2cc(N3CCC(c4c(C)cccc4F)CC3)c(=O)n(Cc3ncccc3C(F)(F)F)c2n1 ; Cc1ncc2cc(C3CCN(c4c(C)cccc4F)CC3)c(=O)n(Cc3ncccc3OC(F)(F)F)c2n1] 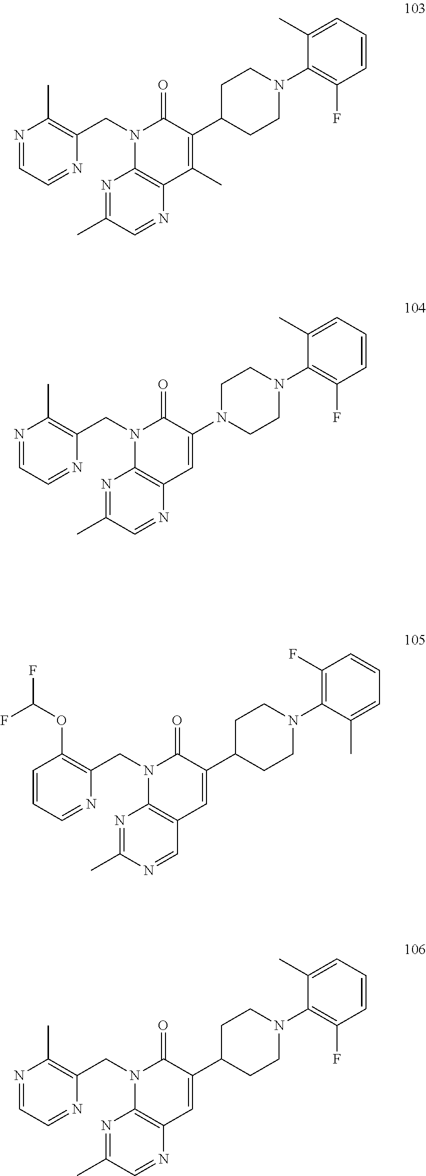 [SMILES: Cc1cnc2c(C)c(C3CCN(c4c(C)cccc4F)CC3)c(=O)n(Cc3nccnc3C)c2n1 ; Cc1cnc2cc(N3CCN(c4c(C)cccc4F)CC3)c(=O)n(Cc3nccnc3C)c2n1 ; Cc1ncc2cc(C3CCN(c4c(C)cccc4F)CC3)c(=O)n(Cc3ncccc3OC(F)F)c2n1 ; Cc1cnc2cc(C3CCN(c4c(C)cccc4F)CC3)c(=O)n(Cc3nccnc3C)c2n1] 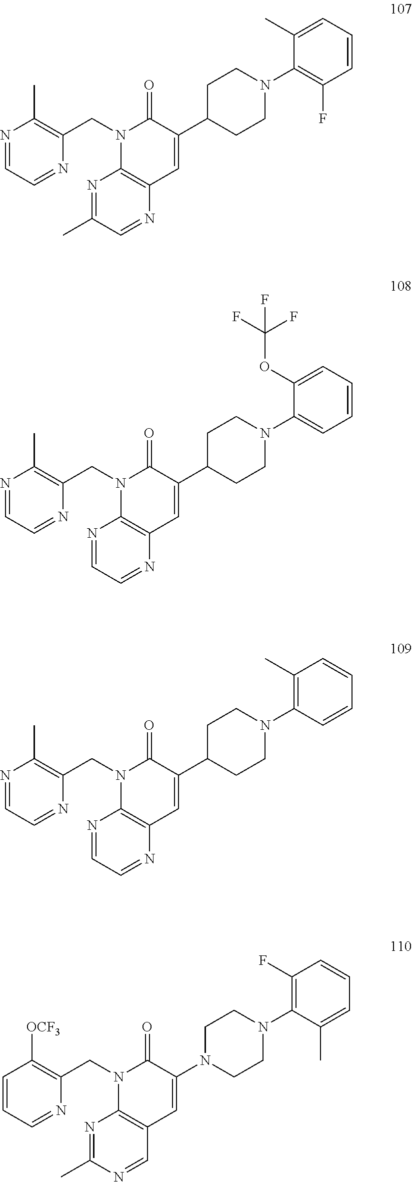 [SMILES: Cc1cnc2cc(C3CCN(c4c(C)cccc4F)CC3)c(=O)n(Cc3nccnc3C)c2n1 ; Cc1nccnc1Cn1c(=O)c(C2CCN(c3ccccc3OC(F)(F)F)CC2)cc2nccnc21 ; Cc1ccccc1N1CCC(c2cc3nccnc3n(Cc3nccnc3C)c2=O)CC1 ; Cc1ncc2cc(N3CCN(c4c(C)cccc4F)CC3)c(=O)n(Cc3ncccc3OC(F)(F)F)c2n1] 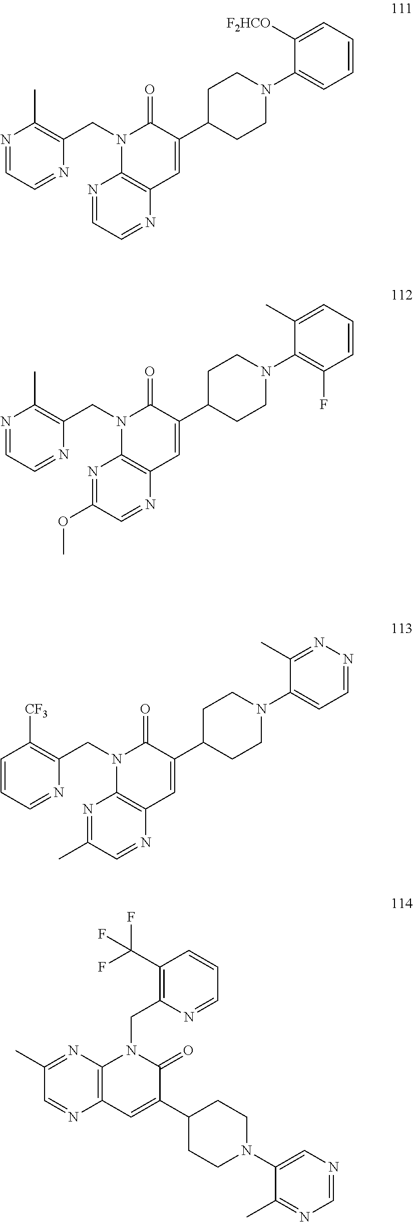 [SMILES: Cc1nccnc1Cn1c(=O)c(C2CCN(c3ccccc3OC(F)F)CC2)cc2nccnc21 ; COc1cnc2cc(C3CCN(c4c(C)cccc4F)CC3)c(=O)n(Cc3nccnc3C)c2n1 ; Cc1cnc2cc(C3CCN(c4ccnnc4C)CC3)c(=O)n(Cc3ncccc3C(F)(F)F)c2n1 ; Cc1cnc2cc(C3CCN(c4cncnc4C)CC3)c(=O)n(Cc3ncccc3C(F)(F)F)c2n1] 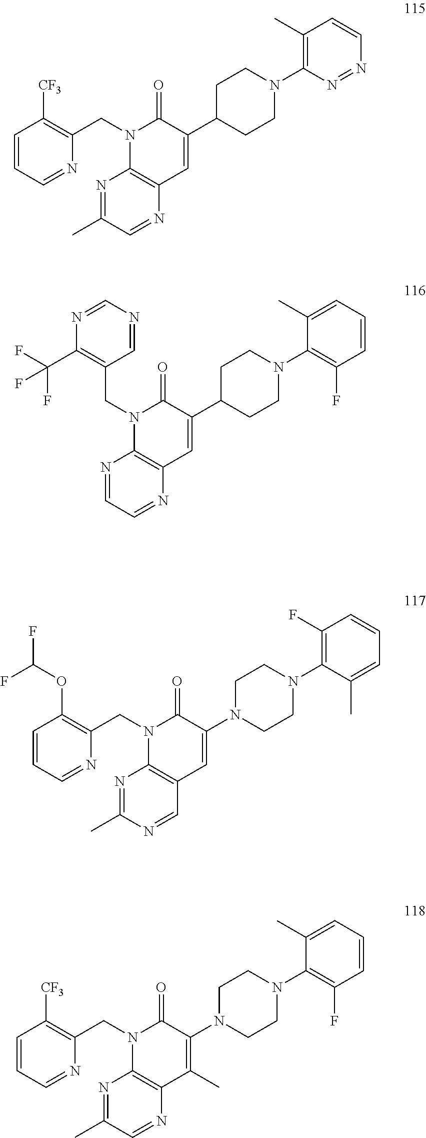 [SMILES: Cc1cnc2cc(C3CCN(c4nnccc4C)CC3)c(=O)n(Cc3ncccc3C(F)(F)F)c2n1 ; Cc1cccc(F)c1N1CCC(c2cc3nccnc3n(Cc3cncnc3C(F)(F)F)c2=O)CC1 ; Cc1ncc2cc(N3CCN(c4c(C)cccc4F)CC3)c(=O)n(Cc3ncccc3OC(F)F)c2n1 ; Cc1cnc2c(C)c(N3CCN(c4c(C)cccc4F)CC3)c(=O)n(Cc3ncccc3C(F)(F)F)c2n1] 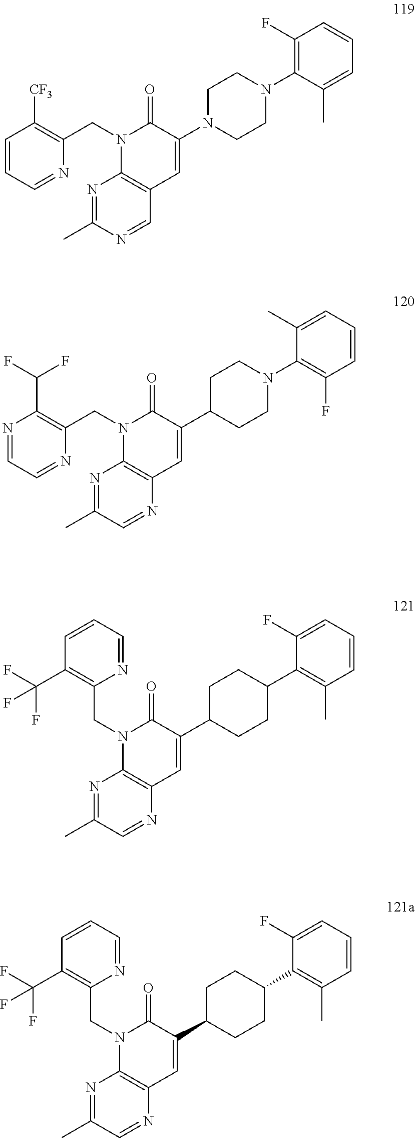 [SMILES: Cc1ncc2cc(N3CCN(c4c(C)cccc4F)CC3)c(=O)n(Cc3ncccc3C(F)(F)F)c2n1 ; Cc1cnc2cc(C3CCN(c4c(C)cccc4F)CC3)c(=O)n(Cc3nccnc3C(F)F)c2n1 ; Cc1cnc2cc(C3CCC(c4c(C)cccc4F)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3C(F)(F)F)c2n1] 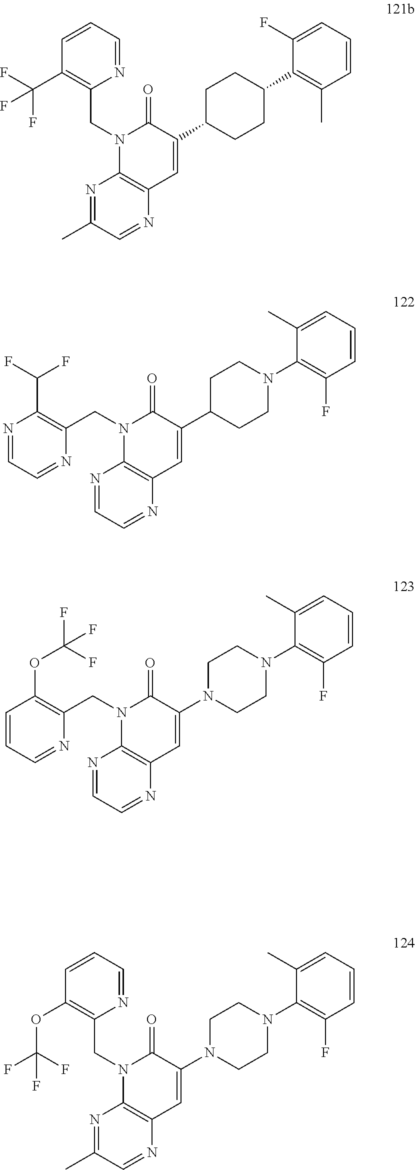 [SMILES: Cc1cnc2cc([C@H]3CC[C@@H](c4c(C)cccc4F)CC3)c(=O)n(Cc3ncccc3C(F)(F)F)c2n1 ; Cc1cccc(F)c1N1CCC(c2cc3nccnc3n(Cc3nccnc3C(F)F)c2=O)CC1 ; Cc1cccc(F)c1N1CCN(c2cc3nccnc3n(Cc3ncccc3OC(F)(F)F)c2=O)CC1 ; Cc1cnc2cc(N3CCN(c4c(C)cccc4F)CC3)c(=O)n(Cc3ncccc3OC(F)(F)F)c2n1] 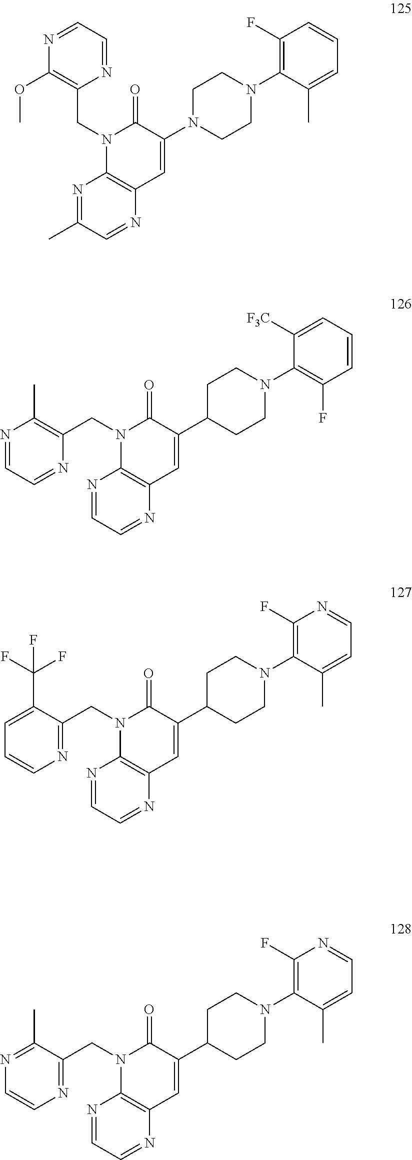 [SMILES: COc1nccnc1Cn1c(=O)c(N2CCN(c3c(C)cccc3F)CC2)cc2ncc(C)nc21 ; Cc1nccnc1Cn1c(=O)c(C2CCN(c3c(F)cccc3C(F)(F)F)CC2)cc2nccnc21 ; Cc1ccnc(F)c1N1CCC(c2cc3nccnc3n(Cc3ncccc3C(F)(F)F)c2=O)CC1 ; Cc1ccnc(F)c1N1CCC(c2cc3nccnc3n(Cc3nccnc3C)c2=O)CC1] 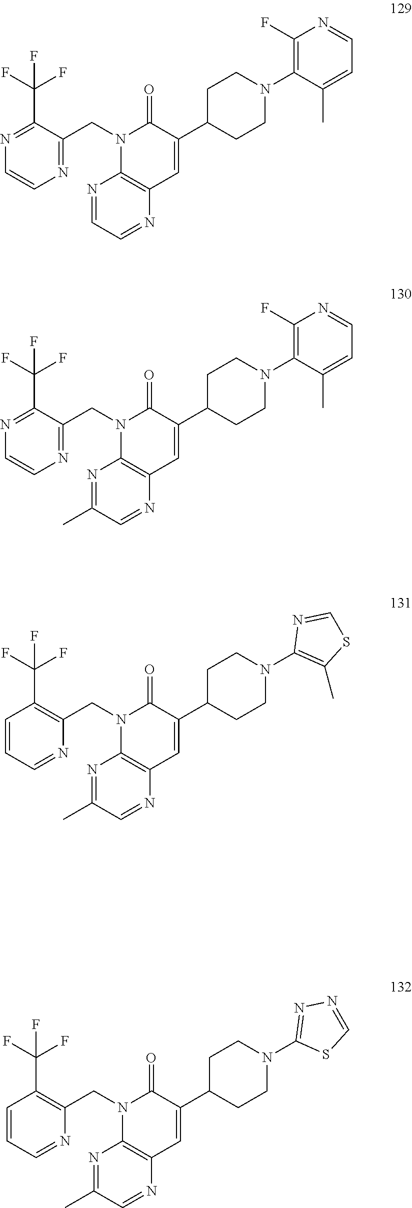 [SMILES: Cc1ccnc(F)c1N1CCC(c2cc3nccnc3n(Cc3nccnc3C(F)(F)F)c2=O)CC1 ; Cc1cnc2cc(C3CCN(c4c(C)ccnc4F)CC3)c(=O)n(Cc3nccnc3C(F)(F)F)c2n1 ; Cc1cnc2cc(C3CCN(c4ncsc4C)CC3)c(=O)n(Cc3ncccc3C(F)(F)F)c2n1 ; Cc1cnc2cc(C3CCN(c4nncs4)CC3)c(=O)n(Cc3ncccc3C(F)(F)F)c2n1] 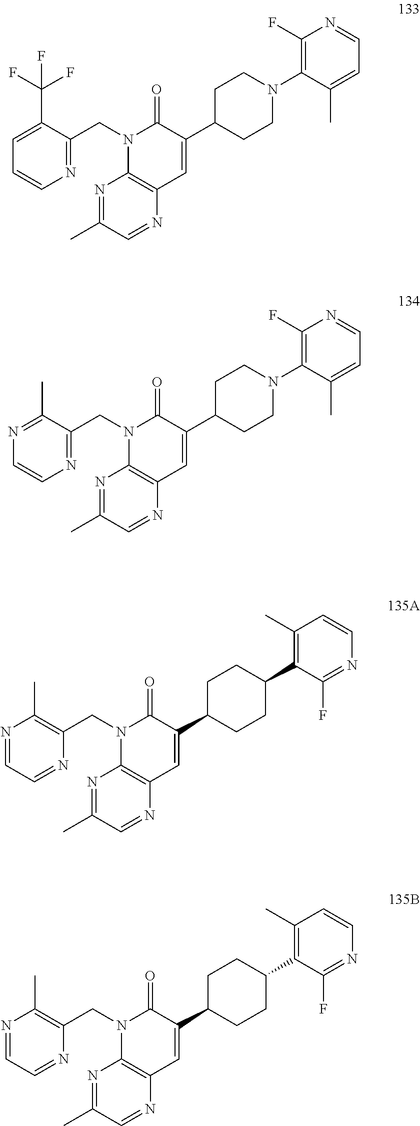 [SMILES: Cc1cnc2cc(C3CCN(c4c(C)ccnc4F)CC3)c(=O)n(Cc3ncccc3C(F)(F)F)c2n1 ; Cc1cnc2cc(C3CCN(c4c(C)ccnc4F)CC3)c(=O)n(Cc3nccnc3C)c2n1 ; Cc1cnc2cc([C@H]3CC[C@@H](c4c(C)ccnc4F)CC3)c(=O)n(Cc3nccnc3C)c2n1 ; Cc1cnc2cc([C@H]3CC[C@H](c4c(C)ccnc4F)CC3)c(=O)n(Cc3nccnc3C)c2n1] 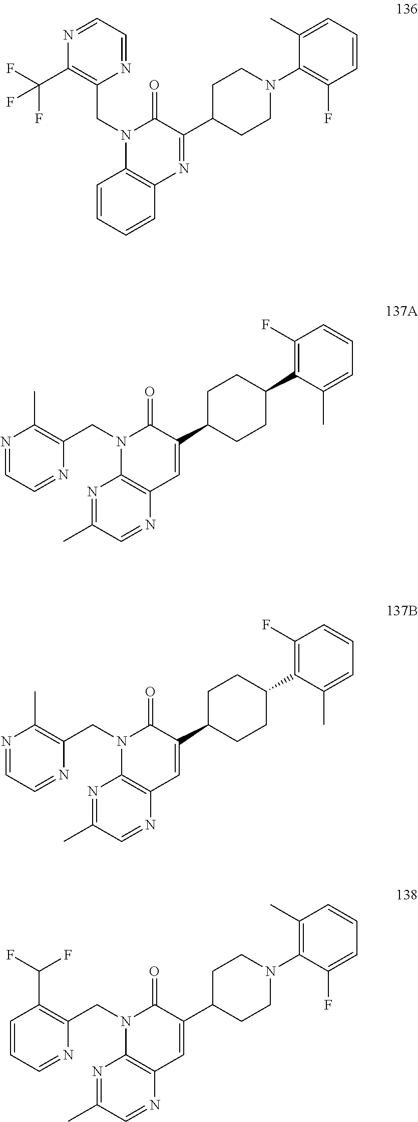 [SMILES: Cc1cccc(F)c1N1CCC(c2nc3ccccc3n(Cc3nccnc3C(F)(F)F)c2=O)CC1 ; Cc1cnc2cc([C@H]3CC[C@@H](c4c(C)cccc4F)CC3)c(=O)n(Cc3nccnc3C)c2n1 ; Cc1cnc2cc([C@H]3CC[C@H](c4c(C)cccc4F)CC3)c(=O)n(Cc3nccnc3C)c2n1 ; Cc1cnc2cc(C3CCN(c4c(C)cccc4F)CC3)c(=O)n(Cc3ncccc3C(F)F)c2n1] 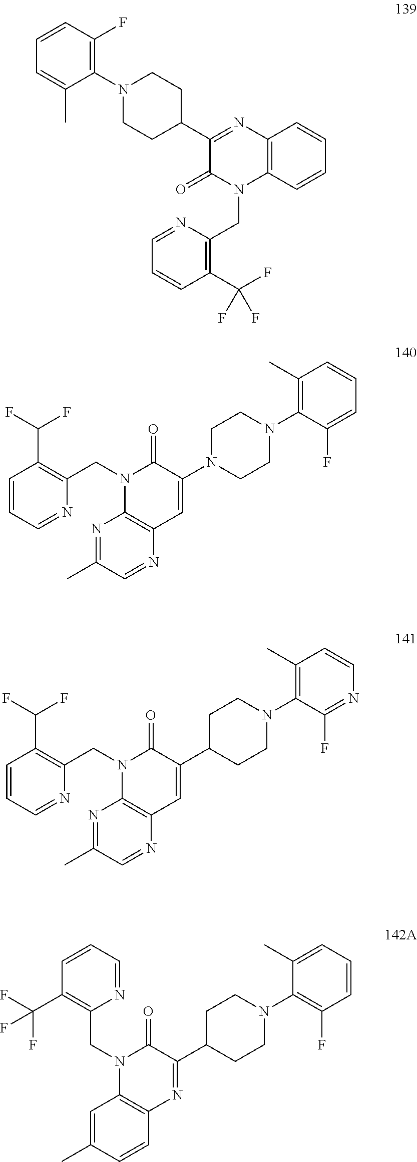 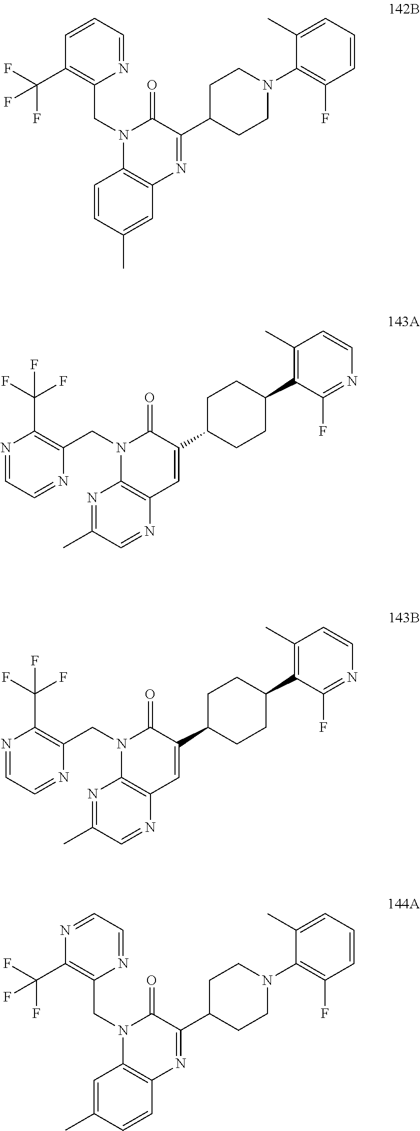 [SMILES: Cc1ccc2c(c1)nc(C1CCN(c3c(C)cccc3F)CC1)c(=O)n2Cc1ncccc1C(F)(F)F ; Cc1cnc2cc([C@H]3CC[C@H](c4c(C)ccnc4F)CC3)c(=O)n(Cc3nccnc3C(F)(F)F)c2n1 ; Cc1cnc2cc([C@H]3CC[C@@H](c4c(C)ccnc4F)CC3)c(=O)n(Cc3nccnc3C(F)(F)F)c2n1 ; Cc1ccc2nc(C3CCN(c4c(C)cccc4F)CC3)c(=O)n(Cc3nccnc3C(F)(F)F)c2c1] 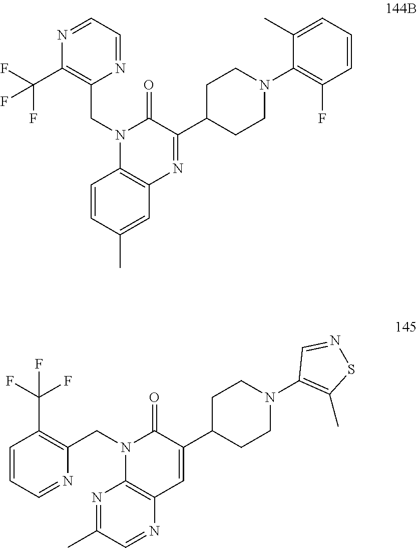 [SMILES: Cc1ccc2c(c1)nc(C1CCN(c3c(C)cccc3F)CC1)c(=O)n2Cc1nccnc1C(F)(F)F ; Cc1cnc2cc(C3CCN(c4cnsc4C)CC3)c(=O)n(Cc3ncccc3C(F)(F)F)c2n1] 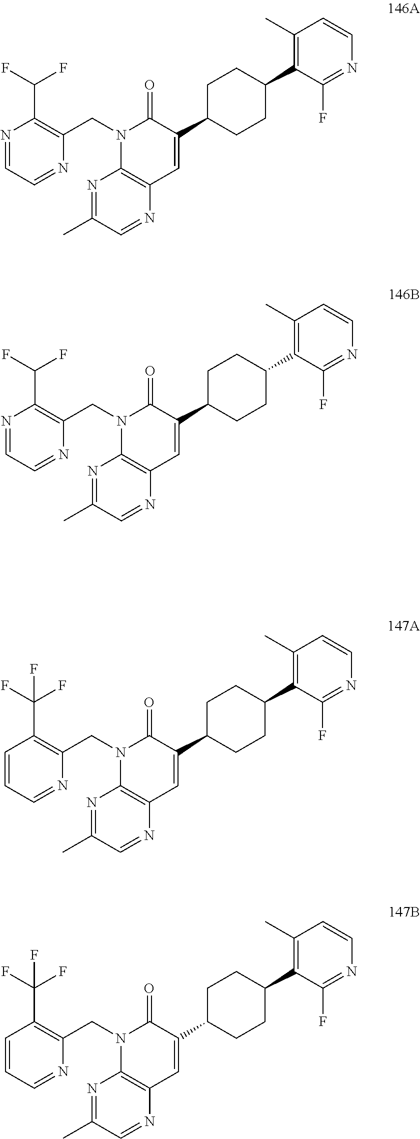 [SMILES: Cc1cnc2cc([C@H]3CC[C@@H](c4c(C)ccnc4F)CC3)c(=O)n(Cc3nccnc3C(F)F)c2n1 ; Cc1cnc2cc([C@H]3CC[C@H](c4c(C)ccnc4F)CC3)c(=O)n(Cc3nccnc3C(F)F)c2n1 ; Cc1cnc2cc([C@H]3CC[C@@H](c4c(C)ccnc4F)CC3)c(=O)n(Cc3ncccc3C(F)(F)F)c2n1 ; Cc1cnc2cc([C@H]3CC[C@H](c4c(C)ccnc4F)CC3)c(=O)n(Cc3ncccc3C(F)(F)F)c2n1] 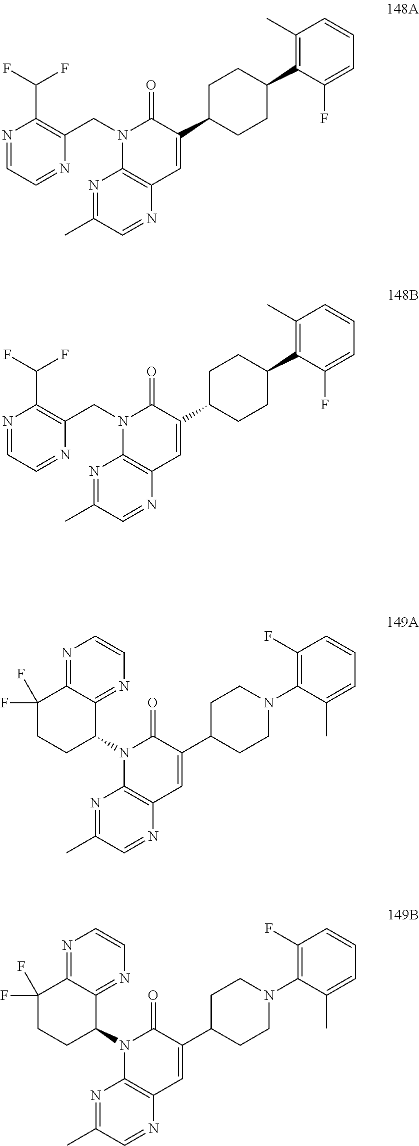 [SMILES: Cc1cnc2cc([C@H]3CC[C@@H](c4c(C)cccc4F)CC3)c(=O)n(Cc3nccnc3C(F)F)c2n1 ; Cc1cnc2cc([C@H]3CC[C@H](c4c(C)cccc4F)CC3)c(=O)n(Cc3nccnc3C(F)F)c2n1 ; Cc1cnc2cc(C3CCN(c4c(C)cccc4F)CC3)c(=O)n([C@@H]3CCC(F)(F)c4nccnc43)c2n1 ; Cc1cnc2cc(C3CCN(c4c(C)cccc4F)CC3)c(=O)n([C@H]3CCC(F)(F)c4nccnc43)c2n1] 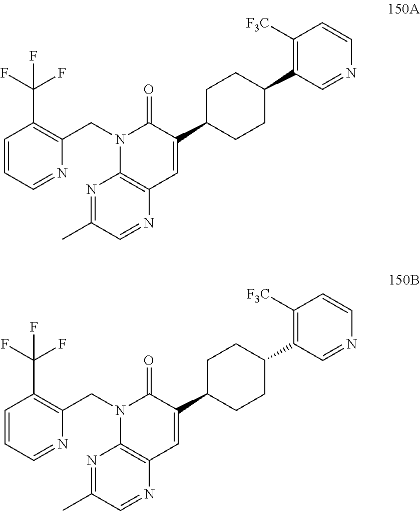 [SMILES: Cc1cnc2cc([C@H]3CC[C@@H](c4cnccc4C(F)(F)F)CC3)c(=O)n(Cc3ncccc3C(F)(F)F)c2n1 ; Cc1cnc2cc([C@H]3CC[C@H](c4cnccc4C(F)(F)F)CC3)c(=O)n(Cc3ncccc3C(F)(F)F)c2n1] 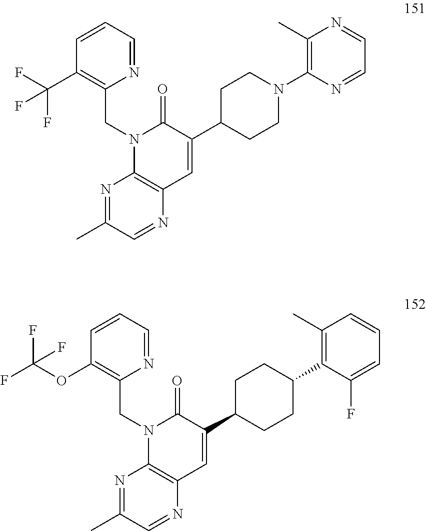 [SMILES: Cc1cnc2cc(C3CCN(c4nccnc4C)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3OC(F)(F)F)c2n1] 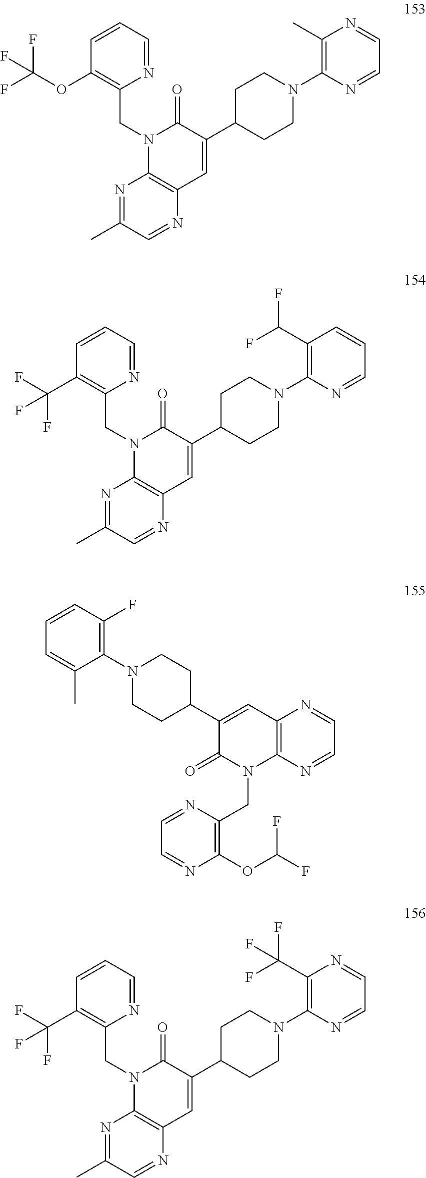 [SMILES: Cc1cnc2cc(C3CCN(c4nccnc4C)CC3)c(=O)n(Cc3ncccc3OC(F)(F)F)c2n1 ; Cc1cnc2cc(C3CCN(c4ncccc4C(F)F)CC3)c(=O)n(Cc3ncccc3C(F)(F)F)c2n1 ; Cc1cccc(F)c1N1CCC(c2cc3nccnc3n(Cc3nccnc3OC(F)F)c2=O)CC1 ; Cc1cnc2cc(C3CCN(c4nccnc4C(F)(F)F)CC3)c(=O)n(Cc3ncccc3C(F)(F)F)c2n1] 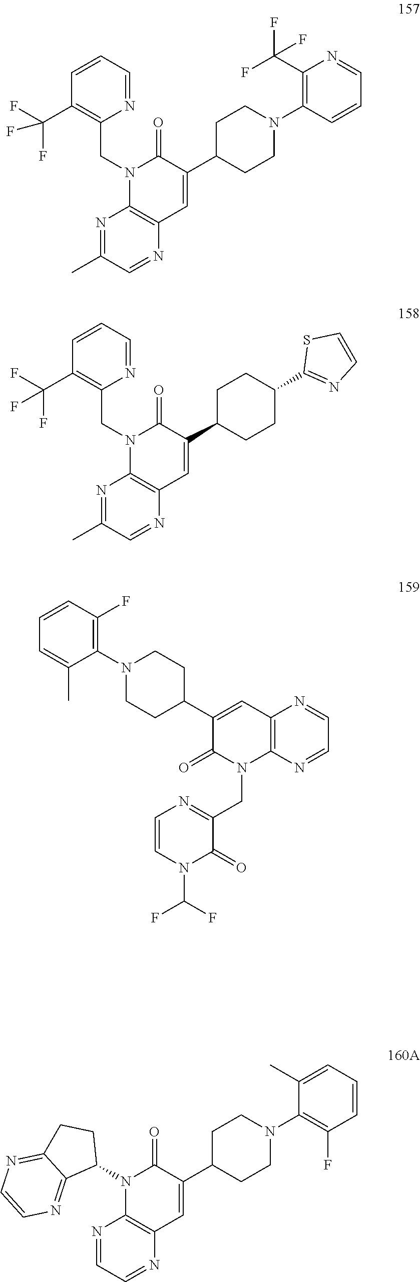 [SMILES: Cc1cnc2cc(C3CCN(c4cccnc4C(F)(F)F)CC3)c(=O)n(Cc3ncccc3C(F)(F)F)c2n1 ; Cc1cnc2cc([C@H]3CC[C@H](c4nccs4)CC3)c(=O)n(Cc3ncccc3C(F)(F)F)c2n1 ; Cc1cccc(F)c1N1CCC(c2cc3nccnc3n(Cc3nccn(C(F)F)c3=O)c2=O)CC1 ; Cc1cccc(F)c1N1CCC(c2cc3nccnc3n([C@H]3CCc4nccnc43)c2=O)CC1] 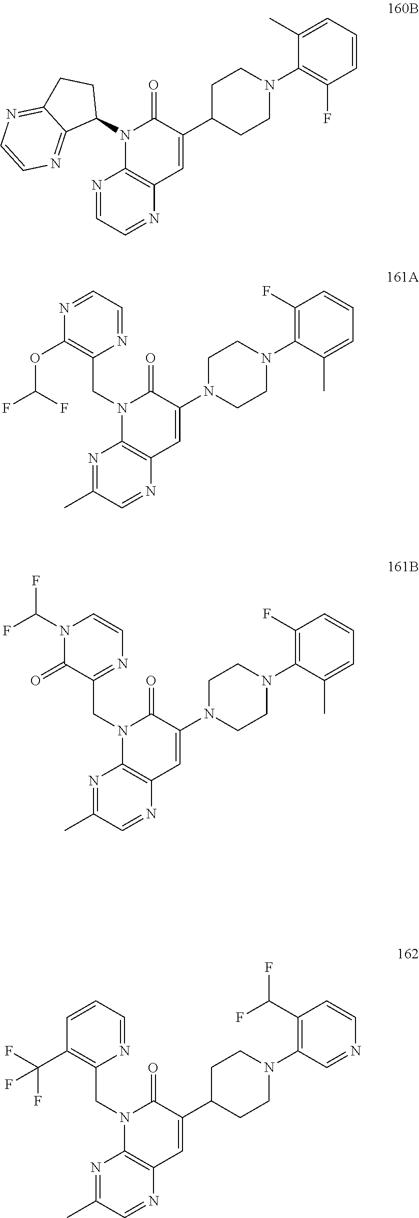 [SMILES: Cc1cccc(F)c1N1CCC(c2cc3nccnc3n([C@@H]3CCc4nccnc43)c2=O)CC1 ; Cc1cnc2cc(N3CCN(c4c(C)cccc4F)CC3)c(=O)n(Cc3nccnc3OC(F)F)c2n1 ; Cc1cnc2cc(N3CCN(c4c(C)cccc4F)CC3)c(=O)n(Cc3nccn(C(F)F)c3=O)c2n1 ; Cc1cnc2cc(C3CCN(c4cnccc4C(F)F)CC3)c(=O)n(Cc3ncccc3C(F)(F)F)c2n1] 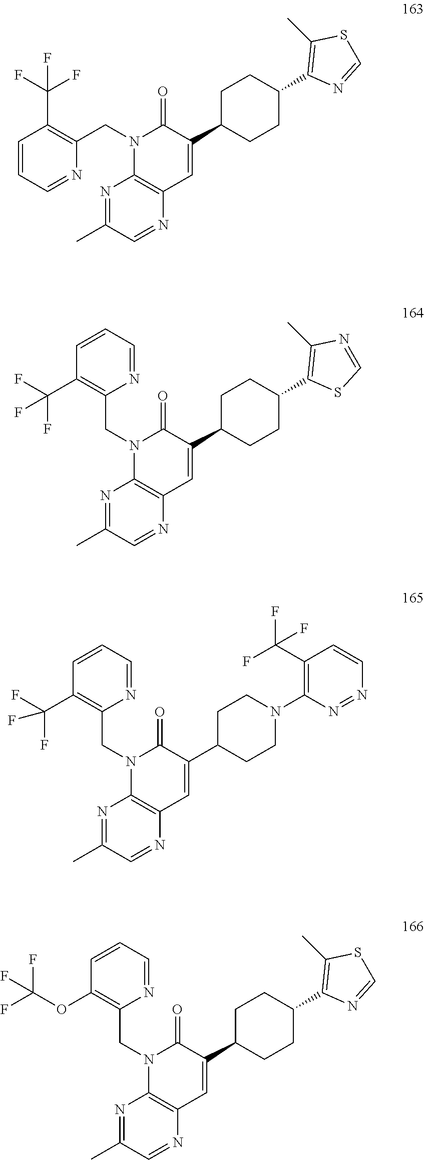 [SMILES: Cc1cnc2cc([C@H]3CC[C@H](c4ncsc4C)CC3)c(=O)n(Cc3ncccc3C(F)(F)F)c2n1 ; Cc1cnc2cc([C@H]3CC[C@H](c4scnc4C)CC3)c(=O)n(Cc3ncccc3C(F)(F)F)c2n1 ; Cc1cnc2cc(C3CCN(c4nnccc4C(F)(F)F)CC3)c(=O)n(Cc3ncccc3C(F)(F)F)c2n1 ; Cc1cnc2cc([C@H]3CC[C@H](c4ncsc4C)CC3)c(=O)n(Cc3ncccc3OC(F)(F)F)c2n1] 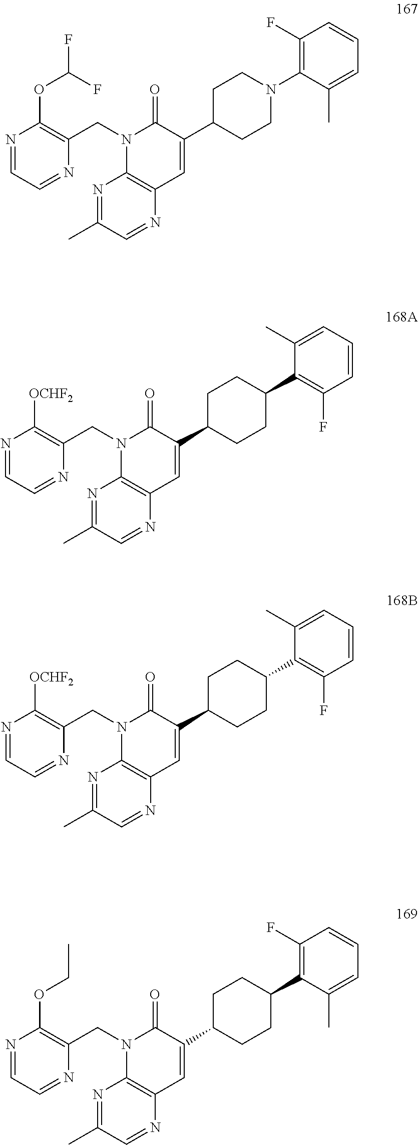 [SMILES: Cc1cnc2cc(C3CCN(c4c(C)cccc4F)CC3)c(=O)n(Cc3nccnc3OC(F)F)c2n1 ; Cc1cnc2cc([C@H]3CC[C@@H](c4c(C)cccc4F)CC3)c(=O)n(Cc3nccnc3OC(F)F)c2n1 ; Cc1cnc2cc([C@H]3CC[C@H](c4c(C)cccc4F)CC3)c(=O)n(Cc3nccnc3OC(F)F)c2n1 ; CCOc1nccnc1Cn1c(=O)c([C@H]2CC[C@H](c3c(C)cccc3F)CC2)cc2ncc(C)nc21] 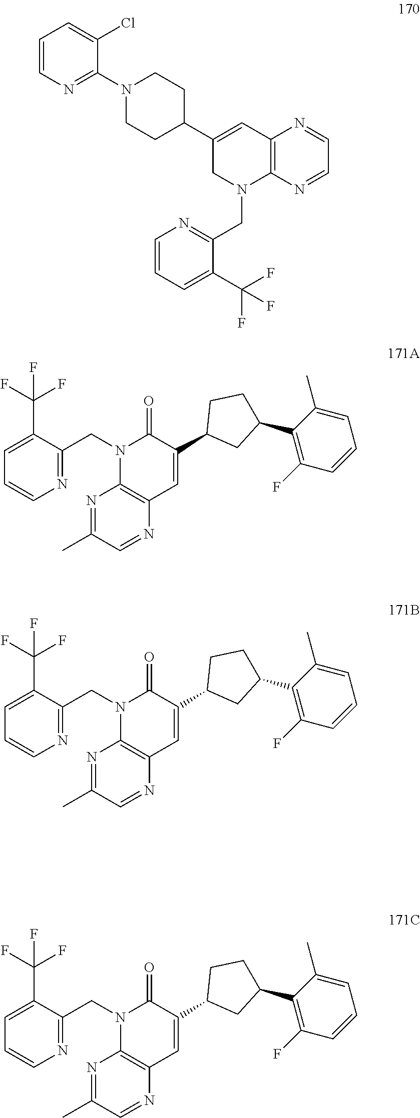 [SMILES: FC(F)(F)c1cccnc1CN1CC(C2CCN(c3ncccc3Cl)CC2)=Cc2nccnc21 ; Cc1cnc2cc([C@H]3CC[C@@H](c4c(C)cccc4F)C3)c(=O)n(Cc3ncccc3C(F)(F)F)c2n1 ; Cc1cnc2cc([C@@H]3CC[C@H](c4c(C)cccc4F)C3)c(=O)n(Cc3ncccc3C(F)(F)F)c2n1 ; Cc1cnc2cc([C@@H]3CC[C@@H](c4c(C)cccc4F)C3)c(=O)n(Cc3ncccc3C(F)(F)F)c2n1] 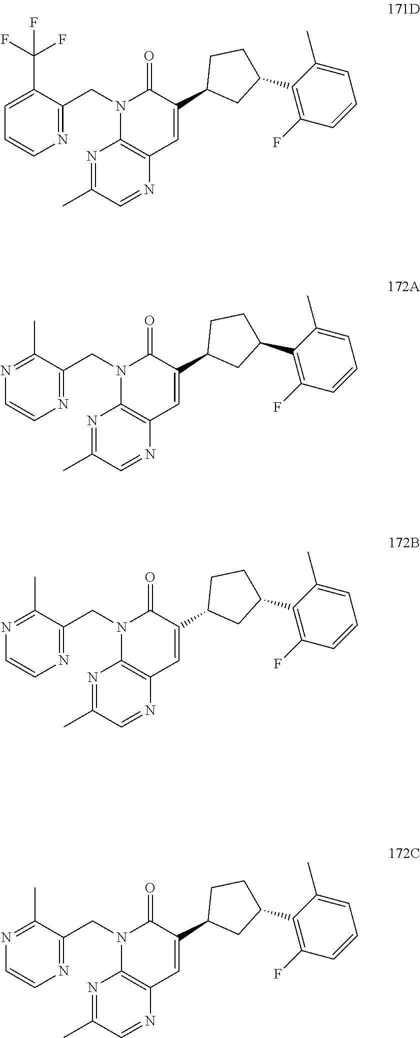 [SMILES: Cc1cnc2cc([C@H]3CC[C@H](c4c(C)cccc4F)C3)c(=O)n(Cc3ncccc3C(F)(F)F)c2n1 ; Cc1cnc2cc([C@H]3CC[C@@H](c4c(C)cccc4F)C3)c(=O)n(Cc3nccnc3C)c2n1 ; Cc1cnc2cc([C@@H]3CC[C@H](c4c(C)cccc4F)C3)c(=O)n(Cc3nccnc3C)c2n1 ; Cc1cnc2cc([C@H]3CC[C@H](c4c(C)cccc4F)C3)c(=O)n(Cc3nccnc3C)c2n1] 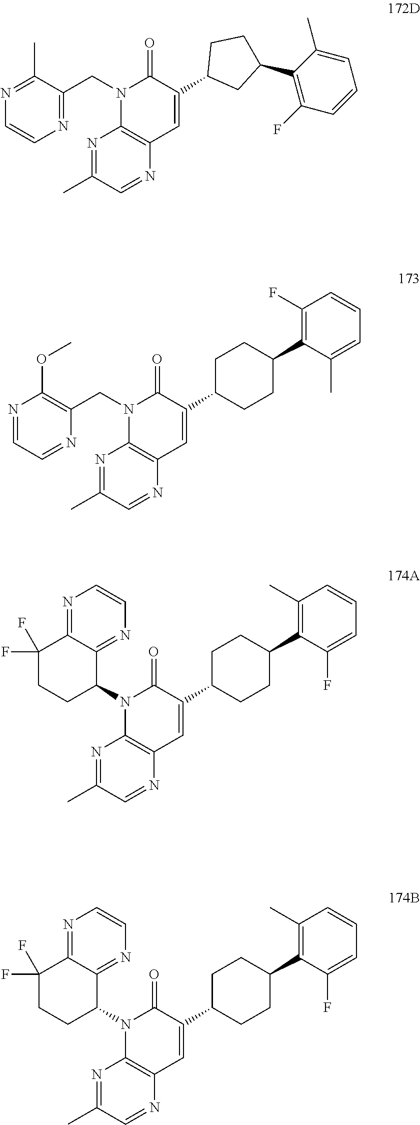 [SMILES: Cc1cnc2cc([C@@H]3CC[C@@H](c4c(C)cccc4F)C3)c(=O)n(Cc3nccnc3C)c2n1 ; COc1nccnc1Cn1c(=O)c([C@H]2CC[C@H](c3c(C)cccc3F)CC2)cc2ncc(C)nc21 ; Cc1cnc2cc([C@H]3CC[C@H](c4c(C)cccc4F)CC3)c(=O)n([C@H]3CCC(F)(F)c4nccnc43)c2n1 ; Cc1cnc2cc([C@H]3CC[C@H](c4c(C)cccc4F)CC3)c(=O)n([C@@H]3CCC(F)(F)c4nccnc43)c2n1] 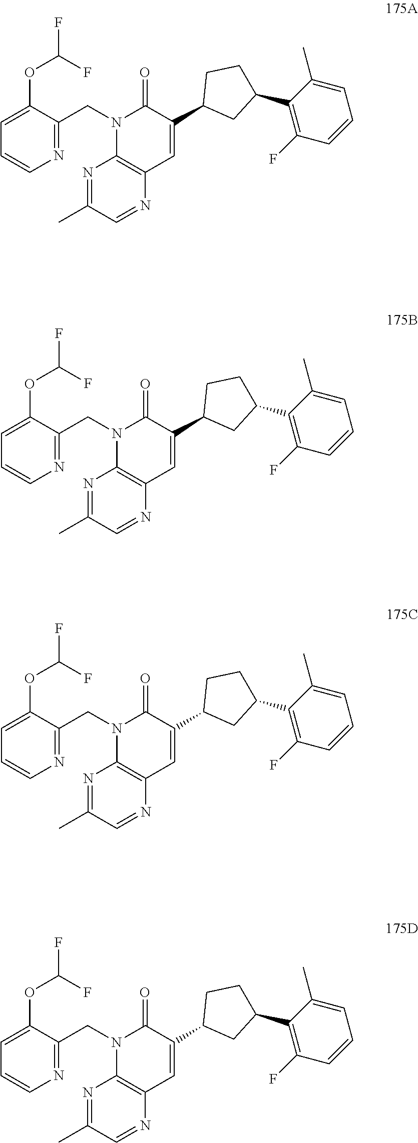 [SMILES: Cc1cnc2cc([C@H]3CC[C@@H](c4c(C)cccc4F)C3)c(=O)n(Cc3ncccc3OC(F)F)c2n1 ; Cc1cnc2cc([C@H]3CC[C@H](c4c(C)cccc4F)C3)c(=O)n(Cc3ncccc3OC(F)F)c2n1 ; Cc1cnc2cc([C@@H]3CC[C@H](c4c(C)cccc4F)C3)c(=O)n(Cc3ncccc3OC(F)F)c2n1 ; Cc1cnc2cc([C@@H]3CC[C@@H](c4c(C)cccc4F)C3)c(=O)n(Cc3ncccc3OC(F)F)c2n1] 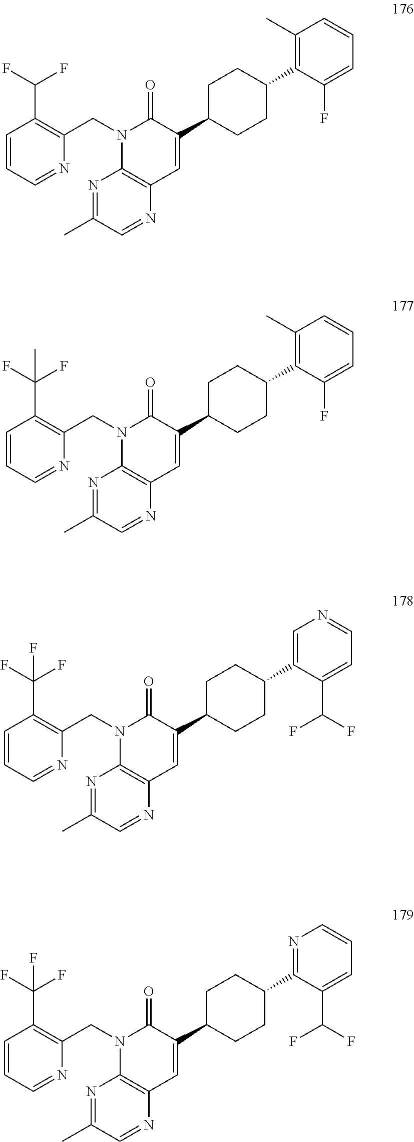 [SMILES: Cc1cnc2cc([C@H]3CC[C@H](c4c(C)cccc4F)CC3)c(=O)n(Cc3ncccc3C(F)F)c2n1 ; Cc1cnc2cc([C@H]3CC[C@H](c4c(C)cccc4F)CC3)c(=O)n(Cc3ncccc3C(C)(F)F)c2n1 ; Cc1cnc2cc([C@H]3CC[C@H](c4cnccc4C(F)F)CC3)c(=O)n(Cc3ncccc3C(F)(F)F)c2n1 ; Cc1cnc2cc([C@H]3CC[C@H](c4ncccc4C(F)F)CC3)c(=O)n(Cc3ncccc3C(F)(F)F)c2n1] 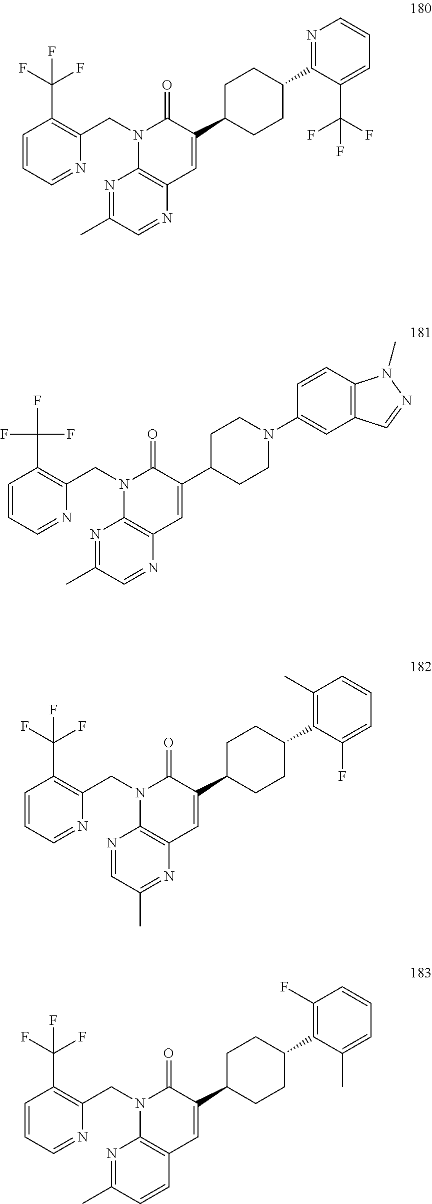 [SMILES: Cc1cnc2cc([C@H]3CC[C@H](c4ncccc4C(F)(F)F)CC3)c(=O)n(Cc3ncccc3C(F)(F)F)c2n1 ; Cc1cnc2cc(C3CCN(c4ccc5c(cnn5C)c4)CC3)c(=O)n(Cc3ncccc3C(F)(F)F)c2n1 ; Cc1cnc2c(cc([C@H]3CC[C@H](c4c(C)cccc4F)CC3)c(=O)n2Cc2ncccc2C(F)(F)F)n1 ; Cc1ccc2cc([C@H]3CC[C@H](c4c(C)cccc4F)CC3)c(=O)n(Cc3ncccc3C(F)(F)F)c2n1] 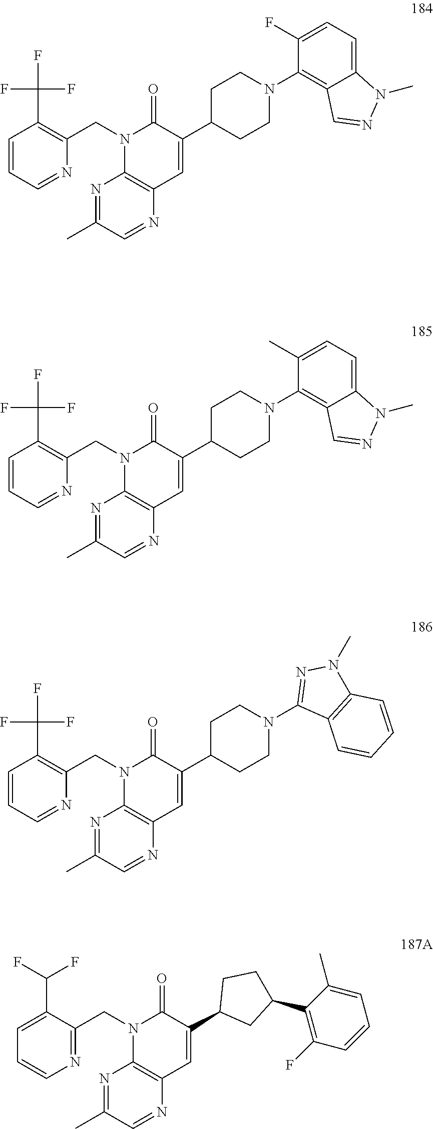 [SMILES: Cc1cnc2cc(C3CCN(c4c(F)ccc5c4cnn5C)CC3)c(=O)n(Cc3ncccc3C(F)(F)F)c2n1 ; Cc1cnc2cc(C3CCN(c4c(C)ccc5c4cnn5C)CC3)c(=O)n(Cc3ncccc3C(F)(F)F)c2n1 ; Cc1cnc2cc(C3CCN(c4nn(C)c5ccccc45)CC3)c(=O)n(Cc3ncccc3C(F)(F)F)c2n1 ; Cc1cnc2cc([C@H]3CC[C@@H](c4c(C)cccc4F)C3)c(=O)n(Cc3ncccc3C(F)F)c2n1] 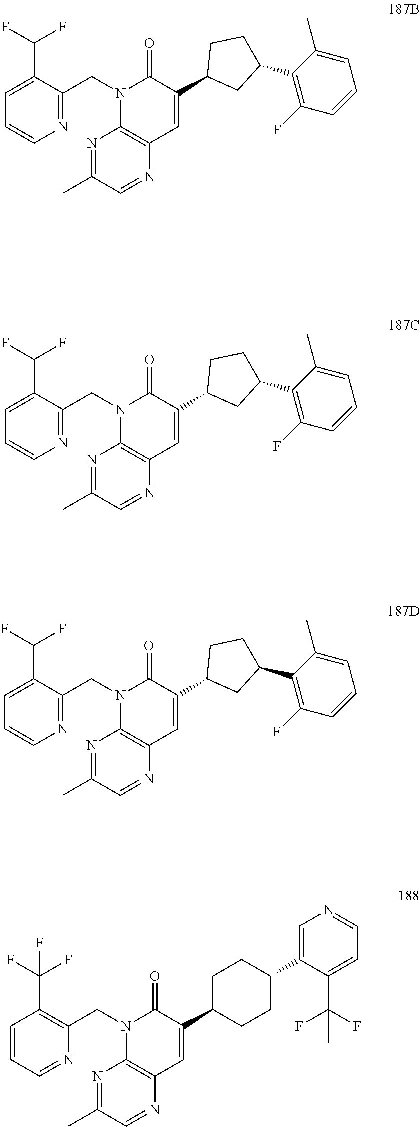 [SMILES: Cc1cnc2cc([C@H]3CC[C@H](c4c(C)cccc4F)C3)c(=O)n(Cc3ncccc3C(F)F)c2n1 ; Cc1cnc2cc([C@@H]3CC[C@H](c4c(C)cccc4F)C3)c(=O)n(Cc3ncccc3C(F)F)c2n1 ; Cc1cnc2cc([C@@H]3CC[C@@H](c4c(C)cccc4F)C3)c(=O)n(Cc3ncccc3C(F)F)c2n1 ; Cc1cnc2cc([C@H]3CC[C@H](c4cnccc4C(C)(F)F)CC3)c(=O)n(Cc3ncccc3C(F)(F)F)c2n1] 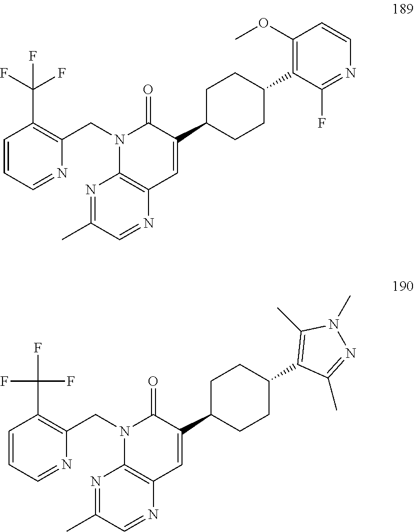 [SMILES: COc1ccnc(F)c1[C@H]1CC[C@H](c2cc3ncc(C)nc3n(Cc3ncccc3C(F)(F)F)c2=O)CC1 ; Cc1cnc2cc([C@H]3CC[C@H](c4c(C)nn(C)c4C)CC3)c(=O)n(Cc3ncccc3C(F)(F)F)c2n1] 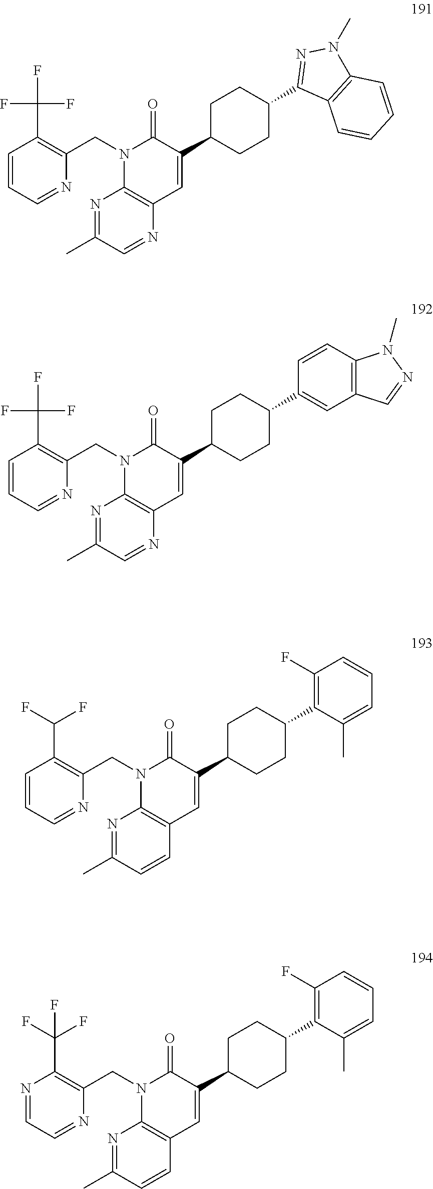 [SMILES: Cc1cnc2cc([C@H]3CC[C@H](c4nn(C)c5ccccc54)CC3)c(=O)n(Cc3ncccc3C(F)(F)F)c2n1 ; Cc1cnc2cc([C@H]3CC[C@H](c4ccc5c(cnn5C)c4)CC3)c(=O)n(Cc3ncccc3C(F)(F)F)c2n1 ; Cc1ccc2cc([C@H]3CC[C@H](c4c(C)cccc4F)CC3)c(=O)n(Cc3ncccc3C(F)F)c2n1 ; Cc1ccc2cc([C@H]3CC[C@H](c4c(C)cccc4F)CC3)c(=O)n(Cc3nccnc3C(F)(F)F)c2n1] 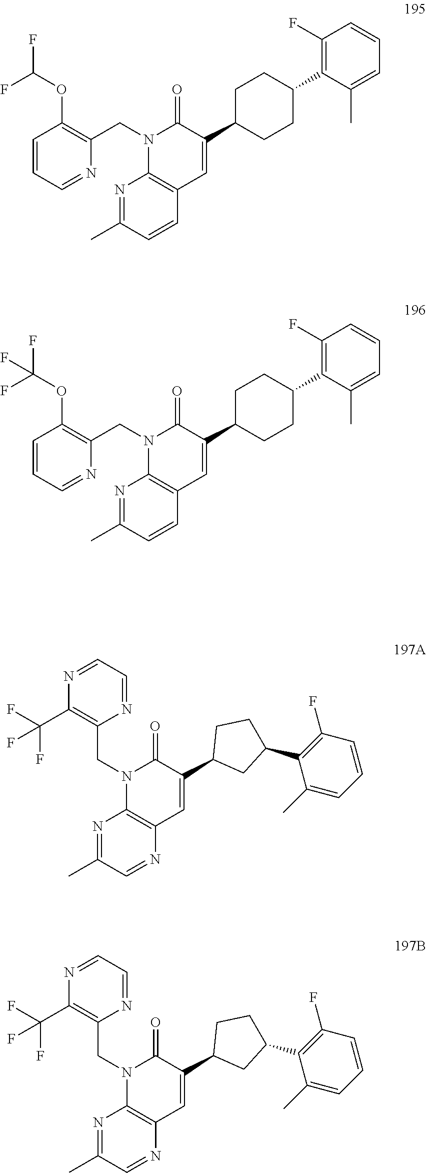 [SMILES: Cc1ccc2cc([C@H]3CC[C@H](c4c(C)cccc4F)CC3)c(=O)n(Cc3ncccc3OC(F)F)c2n1 ; Cc1ccc2cc([C@H]3CC[C@H](c4c(C)cccc4F)CC3)c(=O)n(Cc3ncccc3OC(F)(F)F)c2n1 ; Cc1cnc2cc([C@H]3CC[C@@H](c4c(C)cccc4F)C3)c(=O)n(Cc3nccnc3C(F)(F)F)c2n1 ; Cc1cnc2cc([C@H]3CC[C@H](c4c(C)cccc4F)C3)c(=O)n(Cc3nccnc3C(F)(F)F)c2n1] 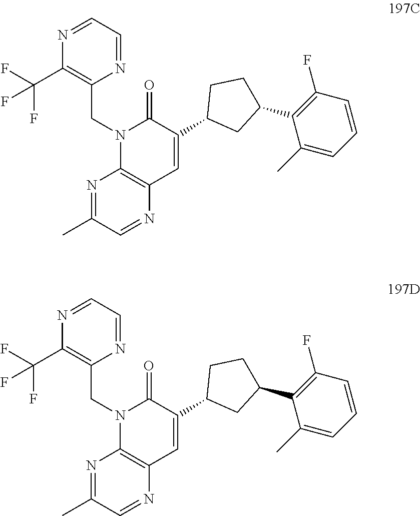 [SMILES: Cc1cnc2cc([C@@H]3CC[C@H](c4c(C)cccc4F)C3)c(=O)n(Cc3nccnc3C(F)(F)F)c2n1 ; Cc1cnc2cc([C@@H]3CC[C@@H](c4c(C)cccc4F)C3)c(=O)n(Cc3nccnc3C(F)(F)F)c2n1] 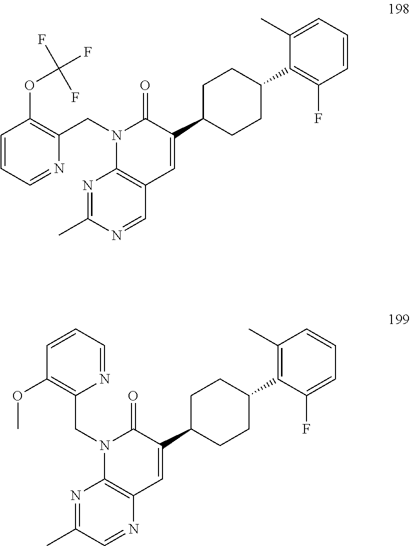 [SMILES: Cc1ncc2cc([C@H]3CC[C@H](c4c(C)cccc4F)CC3)c(=O)n(Cc3ncccc3OC(F)(F)F)c2n1 ; COc1cccnc1Cn1c(=O)c([C@H]2CC[C@H](c3c(C)cccc3F)CC2)cc2ncc(C)nc21] 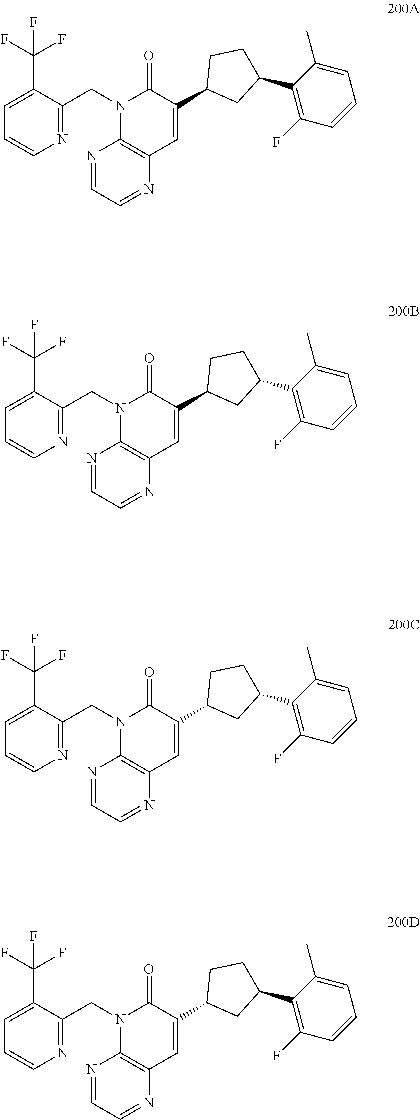 [SMILES: Cc1cccc(F)c1[C@@H]1CC[C@H](c2cc3nccnc3n(Cc3ncccc3C(F)(F)F)c2=O)C1 ; Cc1cccc(F)c1[C@H]1CC[C@H](c2cc3nccnc3n(Cc3ncccc3C(F)(F)F)c2=O)C1 ; Cc1cccc(F)c1[C@H]1CC[C@@H](c2cc3nccnc3n(Cc3ncccc3C(F)(F)F)c2=O)C1 ; Cc1cccc(F)c1[C@@H]1CC[C@@H](c2cc3nccnc3n(Cc3ncccc3C(F)(F)F)c2=O)C1] 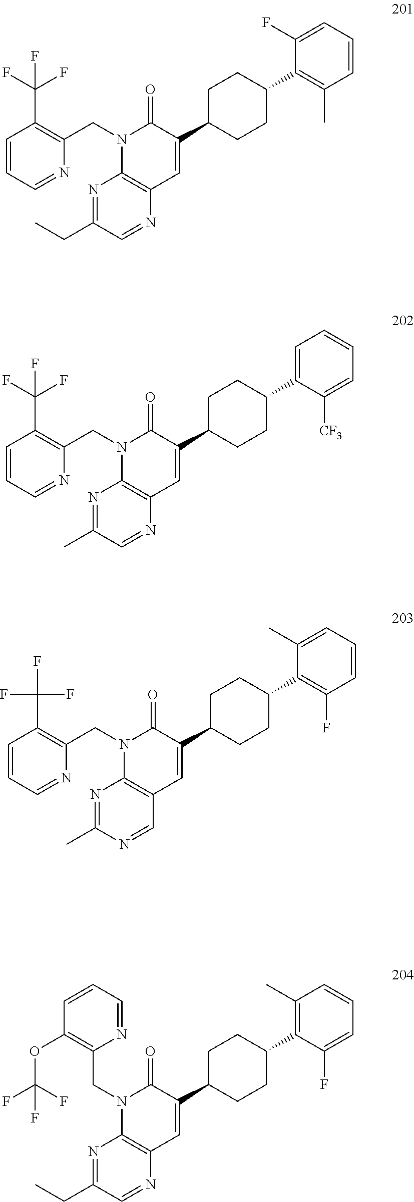 [SMILES: CCc1cnc2cc([C@H]3CC[C@H](c4c(C)cccc4F)CC3)c(=O)n(Cc3ncccc3C(F)(F)F)c2n1 ; Cc1cnc2cc([C@H]3CC[C@H](c4ccccc4C(F)(F)F)CC3)c(=O)n(Cc3ncccc3C(F)(F)F)c2n1 ; Cc1ncc2cc([C@H]3CC[C@H](c4c(C)cccc4F)CC3)c(=O)n(Cc3ncccc3C(F)(F)F)c2n1 ; CCc1cnc2cc([C@H]3CC[C@H](c4c(C)cccc4F)CC3)c(=O)n(Cc3ncccc3OC(F)(F)F)c2n1] 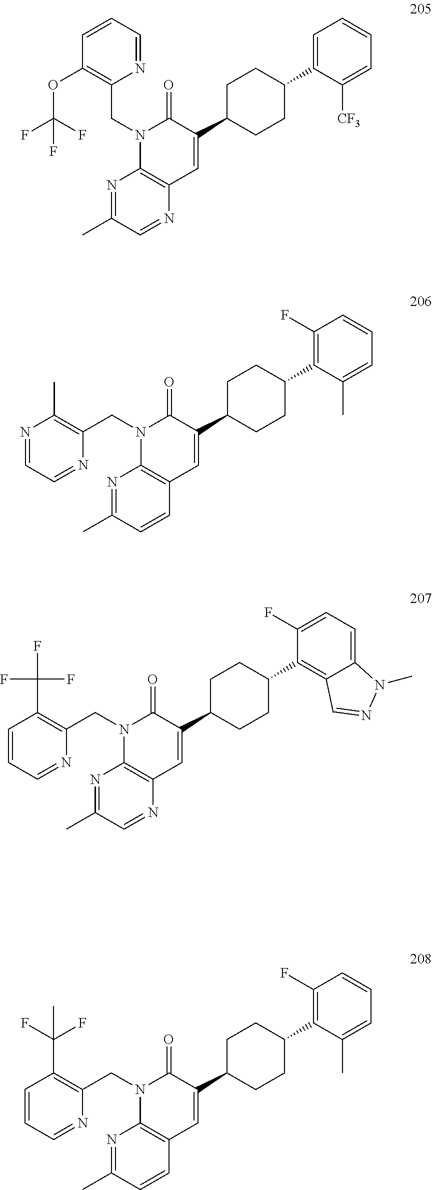 [SMILES: Cc1cnc2cc([C@H]3CC[C@H](c4ccccc4C(F)(F)F)CC3)c(=O)n(Cc3ncccc3OC(F)(F)F)c2n1 ; Cc1ccc2cc([C@H]3CC[C@H](c4c(C)cccc4F)CC3)c(=O)n(Cc3nccnc3C)c2n1 ; Cc1cnc2cc([C@H]3CC[C@H](c4c(F)ccc5c4cnn5C)CC3)c(=O)n(Cc3ncccc3C(F)(F)F)c2n1 ; Cc1ccc2cc([C@H]3CC[C@H](c4c(C)cccc4F)CC3)c(=O)n(Cc3ncccc3C(C)(F)F)c2n1] 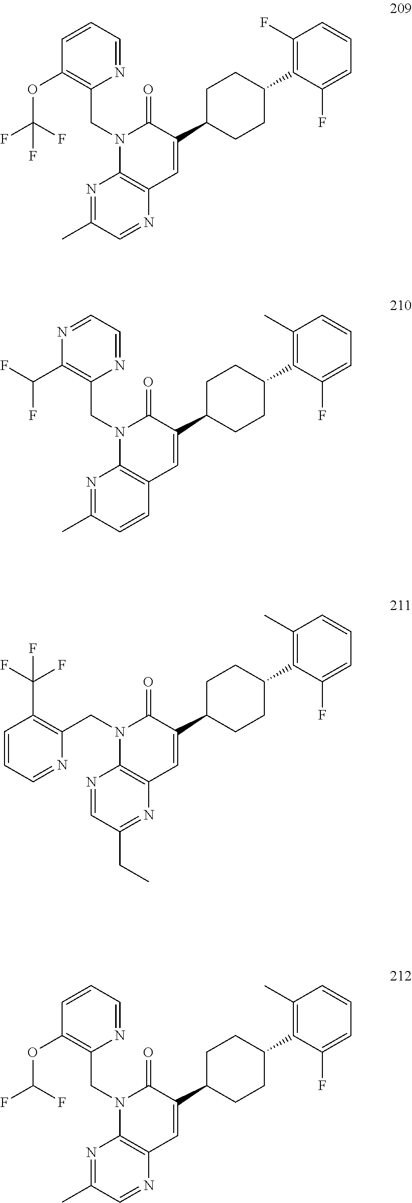 [SMILES: Cc1cnc2cc([C@H]3CC[C@H](c4c(F)cccc4F)CC3)c(=O)n(Cc3ncccc3OC(F)(F)F)c2n1 ; Cc1ccc2cc([C@H]3CC[C@H](c4c(C)cccc4F)CC3)c(=O)n(Cc3nccnc3C(F)F)c2n1 ; CCc1cnc2c(cc([C@H]3CC[C@H](c4c(C)cccc4F)CC3)c(=O)n2Cc2ncccc2C(F)(F)F)n1 ; Cc1cnc2cc([C@H]3CC[C@H](c4c(C)cccc4F)CC3)c(=O)n(Cc3ncccc3OC(F)F)c2n1] 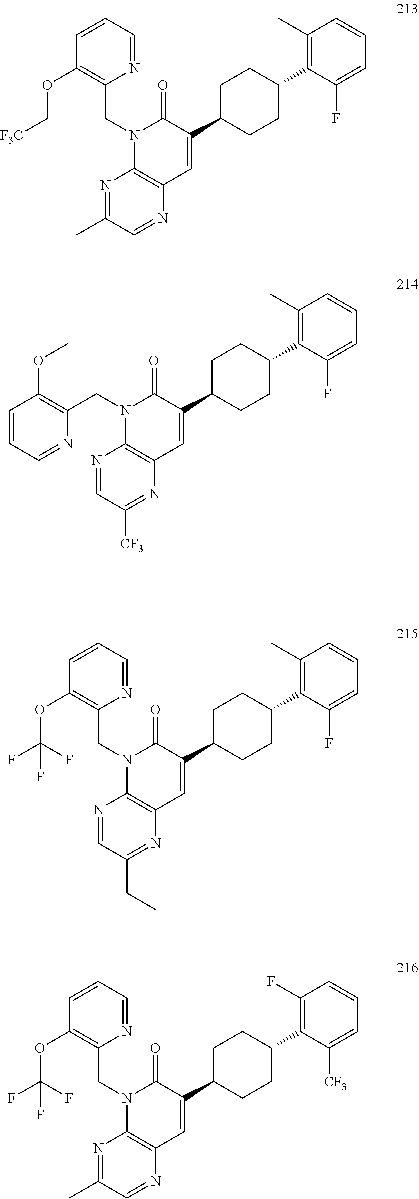 [SMILES: Cc1cnc2cc([C@H]3CC[C@H](c4c(C)cccc4F)CC3)c(=O)n(Cc3ncccc3OCC(F)(F)F)c2n1 ; COc1cccnc1Cn1c(=O)c([C@H]2CC[C@H](c3c(C)cccc3F)CC2)cc2nc(C(F)(F)F)cnc21 ; CCc1cnc2c(cc([C@H]3CC[C@H](c4c(C)cccc4F)CC3)c(=O)n2Cc2ncccc2OC(F)(F)F)n1 ; Cc1cnc2cc([C@H]3CC[C@H](c4c(F)cccc4C(F)(F)F)CC3)c(=O)n(Cc3ncccc3OC(F)(F)F)c2n1] 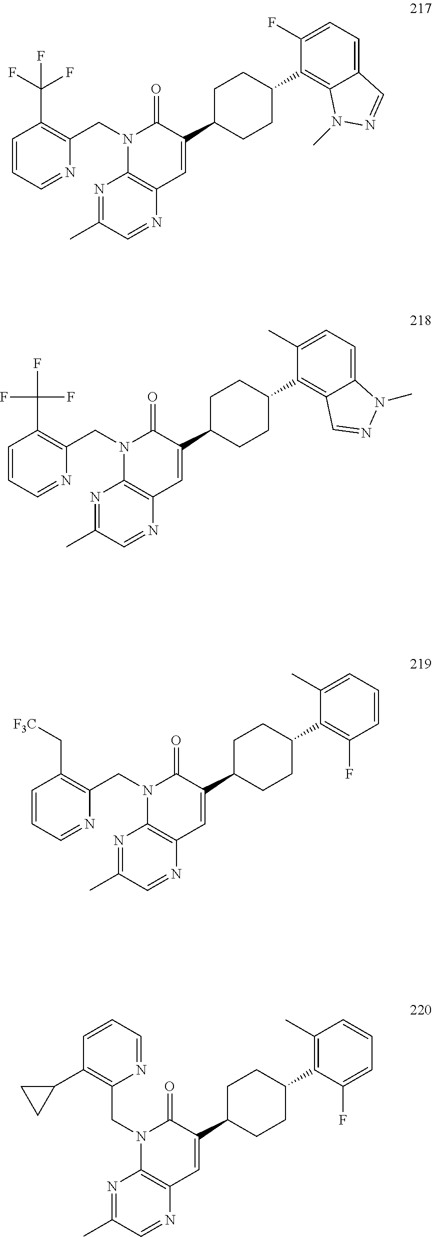 [SMILES: Cc1cnc2cc([C@H]3CC[C@H](c4c(F)ccc5cnn(C)c54)CC3)c(=O)n(Cc3ncccc3C(F)(F)F)c2n1 ; Cc1cnc2cc([C@H]3CC[C@H](c4c(C)ccc5c4cnn5C)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3CC(F)(F)F)c2n1 ; Cc1cnc2cc([C@H]3CC[C@H](c4c(C)cccc4F)CC3)c(=O)n(Cc3ncccc3C3CC3)c2n1] 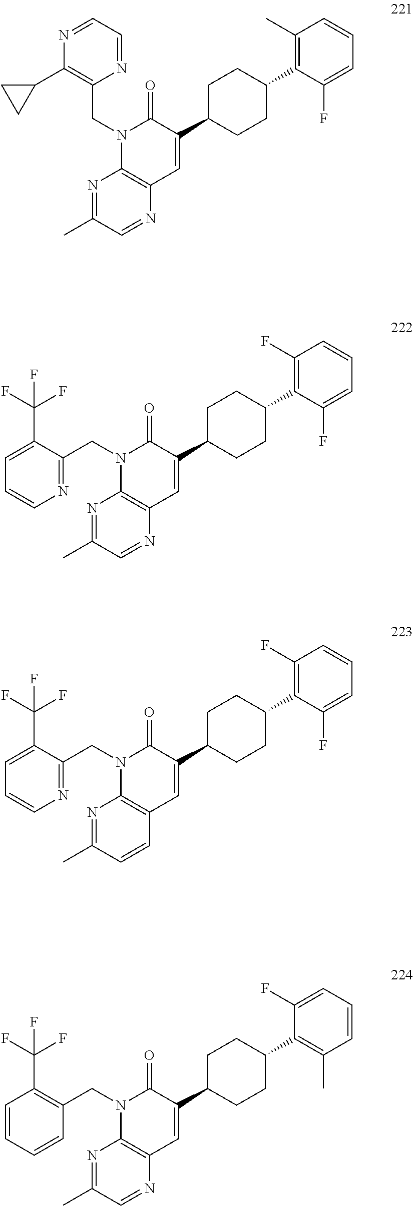 [SMILES: Cc1cnc2cc([C@H]3CC[C@H](c4c(C)cccc4F)CC3)c(=O)n(Cc3nccnc3C3CC3)c2n1 ; Cc1cnc2cc([C@H]3CC[C@H](c4c(F)cccc4F)CC3)c(=O)n(Cc3ncccc3C(F)(F)F)c2n1 ; Cc1ccc2cc([C@H]3CC[C@H](c4c(F)cccc4F)CC3)c(=O)n(Cc3ncccc3C(F)(F)F)c2n1 ; Cc1cnc2cc([C@H]3CC[C@H](c4c(C)cccc4F)CC3)c(=O)n(Cc3ccccc3C(F)(F)F)c2n1] 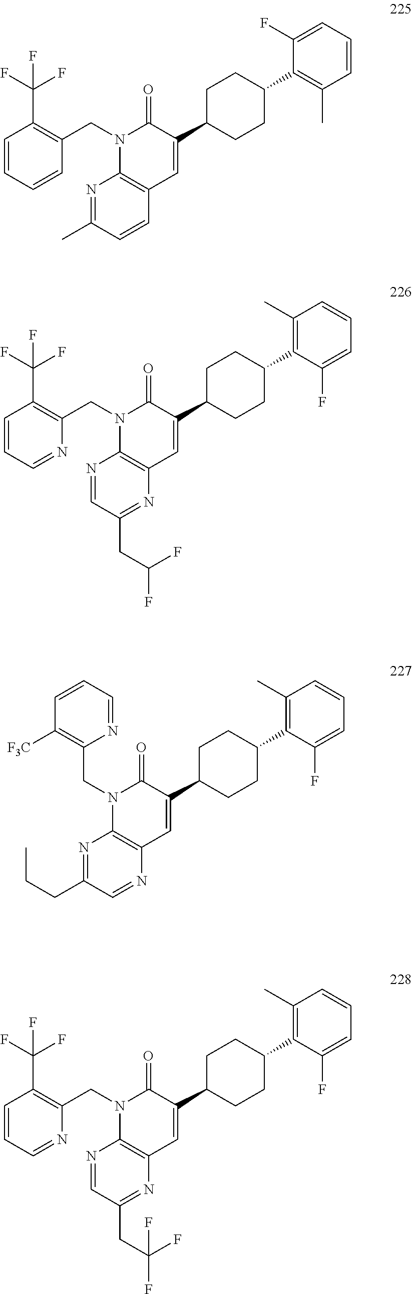 [SMILES: Cc1ccc2cc([C@H]3CC[C@H](c4c(C)cccc4F)CC3)c(=O)n(Cc3ccccc3C(F)(F)F)c2n1 ; Cc1cccc(F)c1[C@H]1CC[C@H](c2cc3nc(CC(F)F)cnc3n(Cc3ncccc3C(F)(F)F)c2=O)CC1 ; CCCc1cnc2cc([C@H]3CC[C@H](c4c(C)cccc4F)CC3)c(=O)n(Cc3ncccc3C(F)(F)F)c2n1 ; Cc1cccc(F)c1[C@H]1CC[C@H](c2cc3nc(CC(F)(F)F)cnc3n(Cc3ncccc3C(F)(F)F)c2=O)CC1] 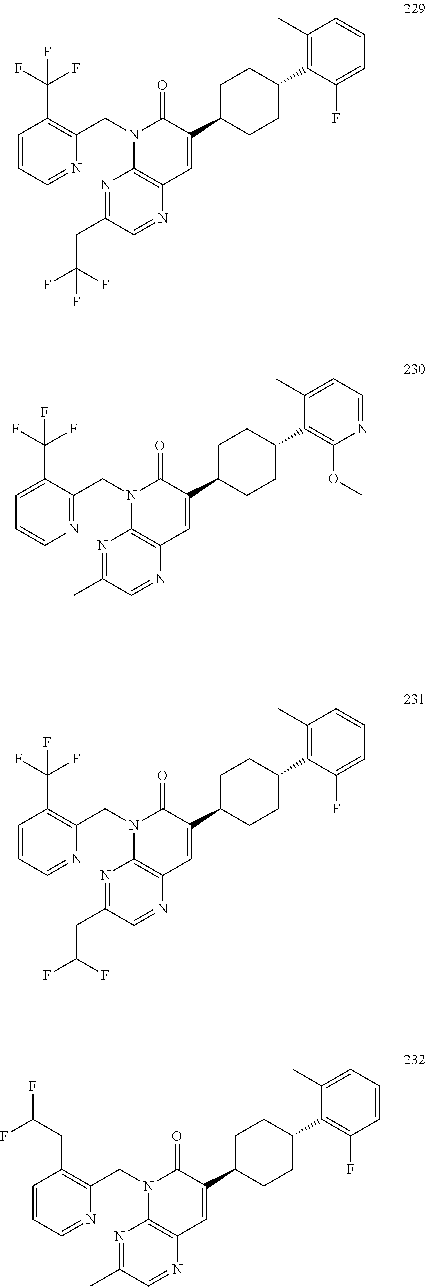 [SMILES: Cc1cccc(F)c1[C@H]1CC[C@H](c2cc3ncc(CC(F)(F)F)nc3n(Cc3ncccc3C(F)(F)F)c2=O)CC1 ; COc1nccc(C)c1[C@H]1CC[C@H](c2cc3ncc(C)nc3n(Cc3ncccc3C(F)(F)F)c2=O)CC1 ; Cc1cccc(F)c1[C@H]1CC[C@H](c2cc3ncc(CC(F)F)nc3n(Cc3ncccc3C(F)(F)F)c2=O)CC1 ; Cc1cnc2cc([C@H]3CC[C@H](c4c(C)cccc4F)CC3)c(=O)n(Cc3ncccc3CC(F)F)c2n1] 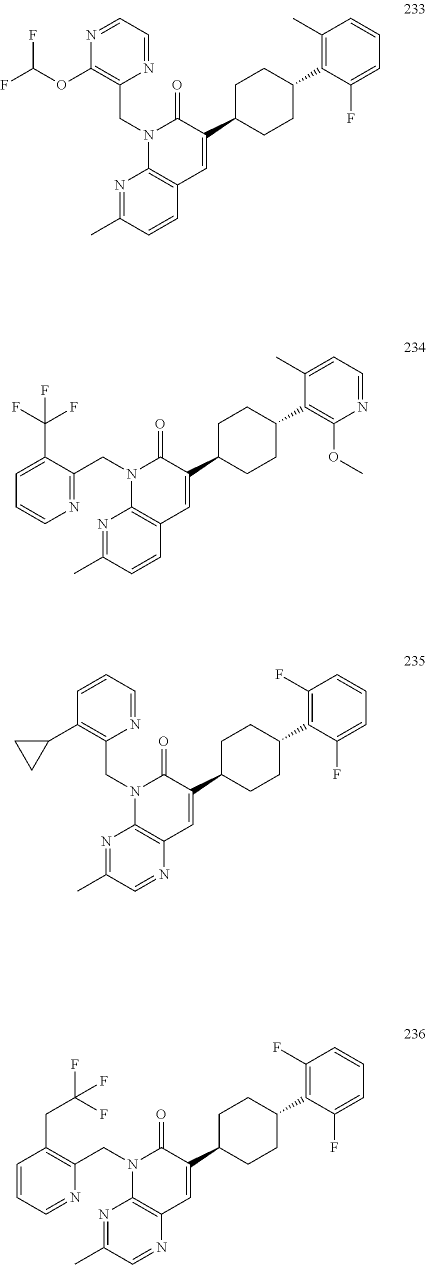 [SMILES: Cc1ccc2cc([C@H]3CC[C@H](c4c(C)cccc4F)CC3)c(=O)n(Cc3nccnc3OC(F)F)c2n1 ; COc1nccc(C)c1[C@H]1CC[C@H](c2cc3ccc(C)nc3n(Cc3ncccc3C(F)(F)F)c2=O)CC1 ; Cc1cnc2cc([C@H]3CC[C@H](c4c(F)cccc4F)CC3)c(=O)n(Cc3ncccc3C3CC3)c2n1 ; Cc1cnc2cc([C@H]3CC[C@H](c4c(F)cccc4F)CC3)c(=O)n(Cc3ncccc3CC(F)(F)F)c2n1] 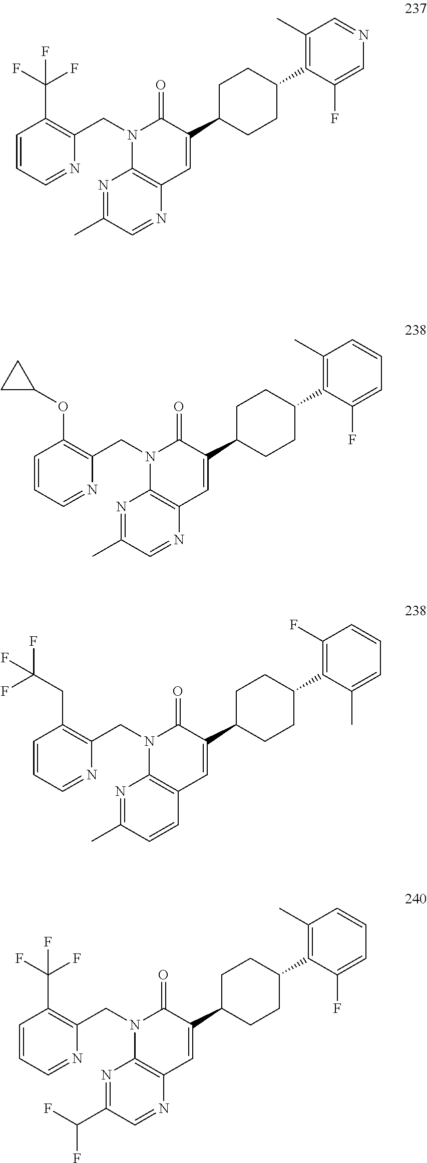 [SMILES: Cc1cnc2cc([C@H]3CC[C@H](c4c(C)cncc4F)CC3)c(=O)n(Cc3ncccc3C(F)(F)F)c2n1 ; Cc1cnc2cc([C@H]3CC[C@H](c4c(C)cccc4F)CC3)c(=O)n(Cc3ncccc3OC3CC3)c2n1 ; Cc1ccc2cc([C@H]3CC[C@H](c4c(C)cccc4F)CC3)c(=O)n(Cc3ncccc3CC(F)(F)F)c2n1 ; Cc1cccc(F)c1[C@H]1CC[C@H](c2cc3ncc(C(F)F)nc3n(Cc3ncccc3C(F)(F)F)c2=O)CC1] 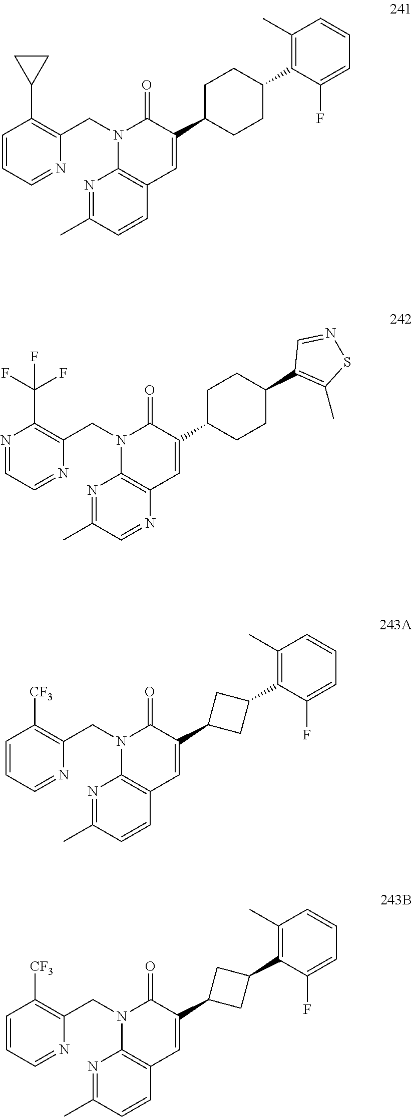 [SMILES: Cc1ccc2cc([C@H]3CC[C@H](c4c(C)cccc4F)CC3)c(=O)n(Cc3ncccc3C3CC3)c2n1 ; Cc1cnc2cc([C@H]3CC[C@H](c4cnsc4C)CC3)c(=O)n(Cc3nccnc3C(F)(F)F)c2n1 ; Cc1ccc2cc([C@H]3C[C@H](c4c(C)cccc4F)C3)c(=O)n(Cc3ncccc3C(F)(F)F)c2n1 ; Cc1ccc2cc([C@H]3C[C@@H](c4c(C)cccc4F)C3)c(=O)n(Cc3ncccc3C(F)(F)F)c2n1] 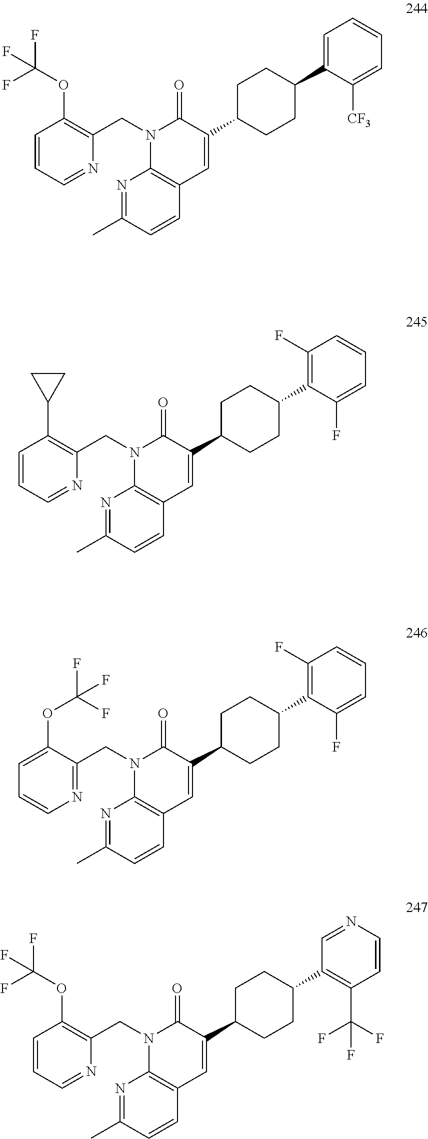 [SMILES: Cc1ccc2cc([C@H]3CC[C@H](c4ccccc4C(F)(F)F)CC3)c(=O)n(Cc3ncccc3OC(F)(F)F)c2n1 ; Cc1ccc2cc([C@H]3CC[C@H](c4c(F)cccc4F)CC3)c(=O)n(Cc3ncccc3C3CC3)c2n1 ; Cc1ccc2cc([C@H]3CC[C@H](c4c(F)cccc4F)CC3)c(=O)n(Cc3ncccc3OC(F)(F)F)c2n1 ; Cc1ccc2cc([C@H]3CC[C@H](c4cnccc4C(F)(F)F)CC3)c(=O)n(Cc3ncccc3OC(F)(F)F)c2n1] 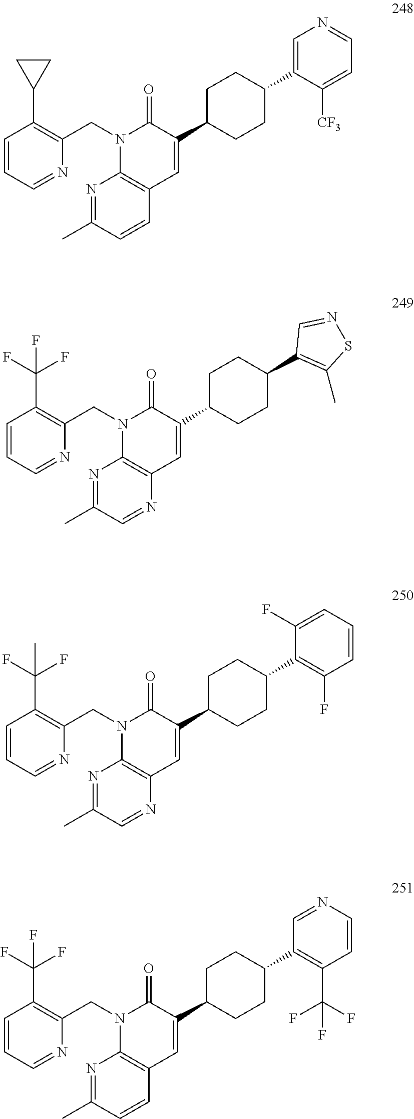 [SMILES: Cc1ccc2cc([C@H]3CC[C@H](c4cnccc4C(F)(F)F)CC3)c(=O)n(Cc3ncccc3C3CC3)c2n1 ; Cc1cnc2cc([C@H]3CC[C@H](c4cnsc4C)CC3)c(=O)n(Cc3ncccc3C(F)(F)F)c2n1 ; Cc1cnc2cc([C@H]3CC[C@H](c4c(F)cccc4F)CC3)c(=O)n(Cc3ncccc3C(C)(F)F)c2n1 ; Cc1ccc2cc([C@H]3CC[C@H](c4cnccc4C(F)(F)F)CC3)c(=O)n(Cc3ncccc3C(F)(F)F)c2n1] 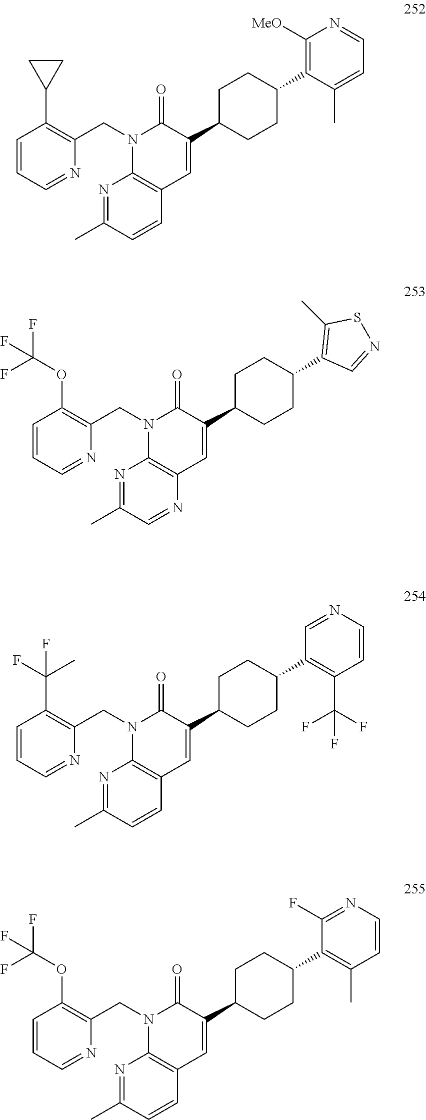 [SMILES: COc1nccc(C)c1[C@H]1CC[C@H](c2cc3ccc(C)nc3n(Cc3ncccc3C3CC3)c2=O)CC1 ; Cc1cnc2cc([C@H]3CC[C@H](c4cnsc4C)CC3)c(=O)n(Cc3ncccc3OC(F)(F)F)c2n1 ; Cc1ccc2cc([C@H]3CC[C@H](c4cnccc4C(F)(F)F)CC3)c(=O)n(Cc3ncccc3C(C)(F)F)c2n1 ; Cc1ccc2cc([C@H]3CC[C@H](c4c(C)ccnc4F)CC3)c(=O)n(Cc3ncccc3OC(F)(F)F)c2n1] 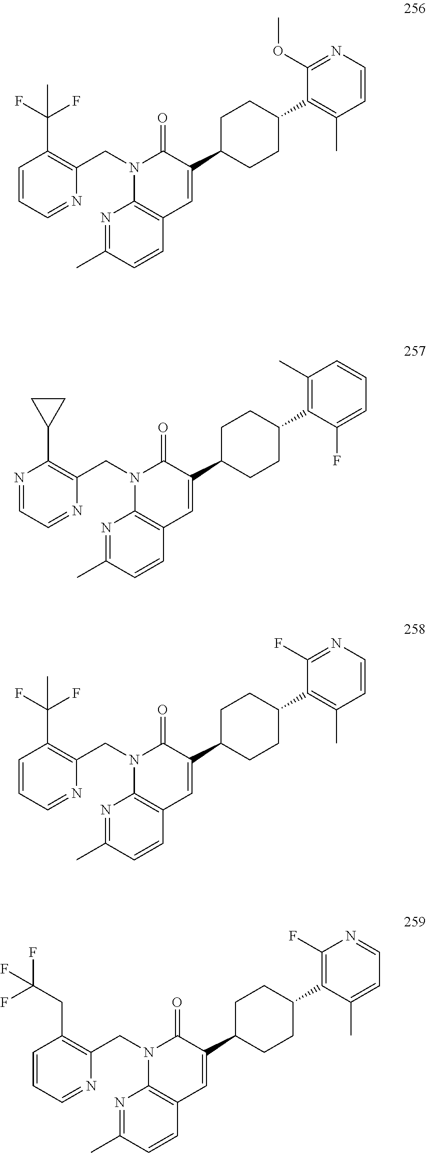 [SMILES: COc1nccc(C)c1[C@H]1CC[C@H](c2cc3ccc(C)nc3n(Cc3ncccc3C(C)(F)F)c2=O)CC1 ; Cc1ccc2cc([C@H]3CC[C@H](c4c(C)cccc4F)CC3)c(=O)n(Cc3nccnc3C3CC3)c2n1 ; Cc1ccc2cc([C@H]3CC[C@H](c4c(C)ccnc4F)CC3)c(=O)n(Cc3ncccc3C(C)(F)F)c2n1 ; Cc1ccc2cc([C@H]3CC[C@H](c4c(C)ccnc4F)CC3)c(=O)n(Cc3ncccc3CC(F)(F)F)c2n1] 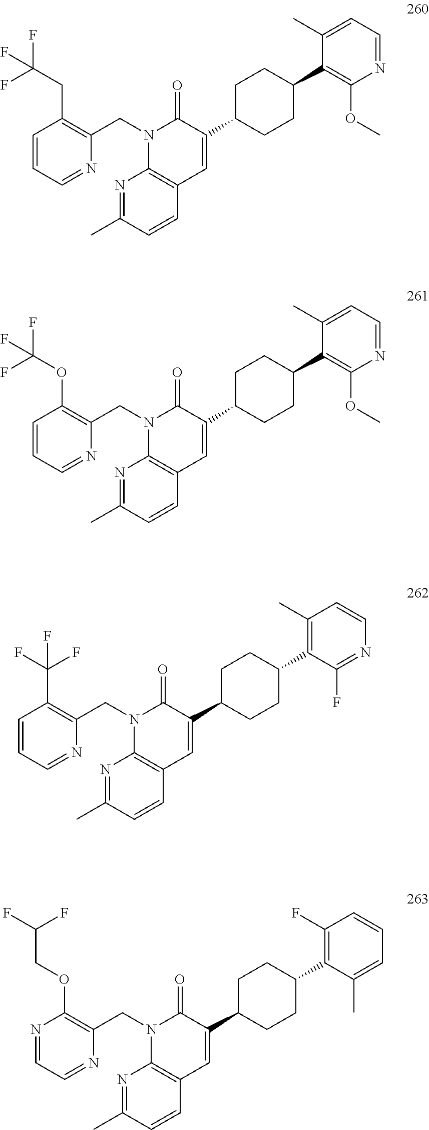 [SMILES: COc1nccc(C)c1[C@H]1CC[C@H](c2cc3ccc(C)nc3n(Cc3ncccc3CC(F)(F)F)c2=O)CC1 ; COc1nccc(C)c1[C@H]1CC[C@H](c2cc3ccc(C)nc3n(Cc3ncccc3OC(F)(F)F)c2=O)CC1 ; Cc1ccc2cc([C@H]3CC[C@H](c4c(C)ccnc4F)CC3)c(=O)n(Cc3ncccc3C(F)(F)F)c2n1 ; Cc1ccc2cc([C@H]3CC[C@H](c4c(C)cccc4F)CC3)c(=O)n(Cc3nccnc3OCC(F)F)c2n1] 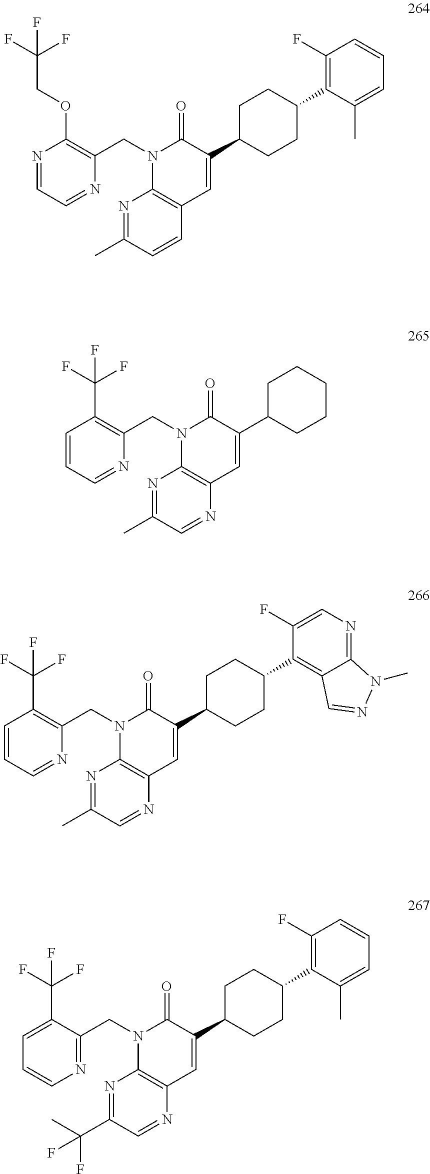 [SMILES: Cc1ccc2cc([C@H]3CC[C@H](c4c(C)cccc4F)CC3)c(=O)n(Cc3nccnc3OCC(F)(F)F)c2n1 ; Cc1cnc2cc(C3CCCCC3)c(=O)n(Cc3ncccc3C(F)(F)F)c2n1 ; Cc1cnc2cc([C@H]3CC[C@H](c4c(F)cnc5c4cnn5C)CC3)c(=O)n(Cc3ncccc3C(F)(F)F)c2n1 ; Cc1cccc(F)c1[C@H]1CC[C@H](c2cc3ncc(C(C)(F)F)nc3n(Cc3ncccc3C(F)(F)F)c2=O)CC1] 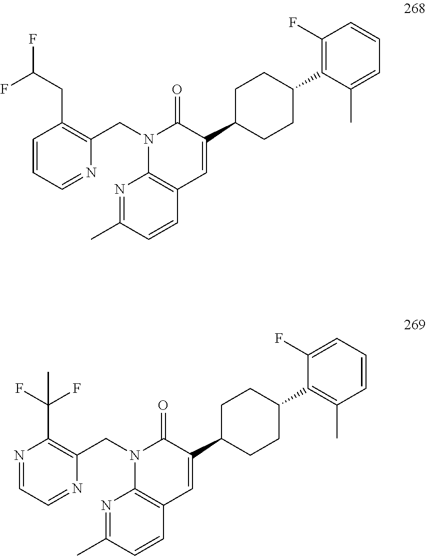 [SMILES: Cc1ccc2cc([C@H]3CC[C@H](c4c(C)cccc4F)CC3)c(=O)n(Cc3ncccc3CC(F)F)c2n1 ; Cc1ccc2cc([C@H]3CC[C@H](c4c(C)cccc4F)CC3)c(=O)n(Cc3nccnc3C(C)(F)F)c2n1]
or a pharmaceutically acceptable salt thereof.

93. A pharmaceutical composition comprising a compound of any of claims 1-92, or a pharmaceutically acceptable salt thereof, and a pharmaceutically acceptable excipient.

94. A method of treating a disease or disorder in a subject in need thereof, the method comprising administering an effective amount of a compound of any of claims 1-92, or pharmaceutically acceptable salt thereof, or a pharmaceutical composition of claim 93.

95. The method of claim 94, wherein the disease or disorder is associated with C5aR1.

96. The method of claim 94 or 95, wherein the disease or disorder is cancer, an infectious disease, an autoimmune disease or disorder, an inflammatory disease or disorder, a cardiovascular disease or disorder, a cerebrovascular disease or disorder, a vasculitis disease or disorder, or a neurodegenerative disease or disorder.

97. The method of claim 96, wherein the disease or disorder is a neurological disease or disorder.

98. A method of antagonizing C5aR1, the method comprising contacting C5aR1 with a compound of any of claims 1-92, or pharmaceutically acceptable salt thereof, or a pharmaceutical composition of claim 93.

99. The method of claim 98, wherein the contacting is in vitro.

100. The method of claim 98, wherein the contacting is in vivo.

101. A kit comprising a compound of any of claims 1-92, or a pharmaceutically acceptable salt thereof, or a pharmaceutical composition of claim 93, and instructions for administering the compound or pharmaceutical composition to a subject in need thereof.
